(19) United States
(12) Patent Application Publication
(10) Pub. No.: US 2023/0002420 A1
(43) Pub. Date: Jan. 5, 2023

(54) ORGANIC LIGHT-EMITTING ELEMENT

(71) Applicant: LG CHEM, LTD., Seoul (KR)

(72) Inventors: Sung Jae LEE, Daejeon (KR); Sujeong GEUM, Daejeon (KR); Moung Gon KIM, Daejeon (KR); Sung Kil HONG, Daejeon (KR); Younghan LEE, Daejeon (KR)

(21) Appl. No.: 17/765,096

(22) PCT Filed: Nov. 30, 2020
(86) PCT No.: PCT/KR2020/017321

§ 371 Date: Mar. 30, 2022

(30) Foreign Application Priority Data
Nov. 29, 2019 (KR) 10-2019-0156836

(51) Int. Cl. C07F 5/02; C07F 7/08; C07C 211/54; C07D 307/91; C07D 333/76; C07C 15/28; H01L 51/00; H01L 51/50

(52) U.S. Cl. CPC C07F 5/027; C07F 7/0812; C07C 211/54; C07D 307/91; C07D 333/76; C07C 15/28; H01L 51/0052; H01L 51/0059; H01L 51/008; H01L 51/0073; H01L 51/0074; H01L 51/009; H01L 51/5056; H01L 51/5072; H01L 51/5088; H01L 51/5092; C07B 2200/05

(57) ABSTRACT

Provided is an organic light emitting device including a light emitting layer comprising a compound of Chemical Formula 1 and a first organic material layer comprising a compound of Chemical Formula 2:

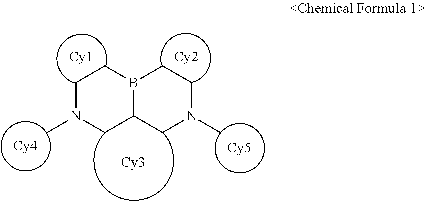

wherein:
Cy1 to Cy5 are each independently one selected from among a substituted or unsubstituted: aromatic hydrocarbon ring, aliphatic hydrocarbon ring, and aromatic hetero ring, or a ring in which two or more rings selected from the above group are fused,
one or more of Cy1 to Cy5 are a ring of Chemical Formula 1-A:

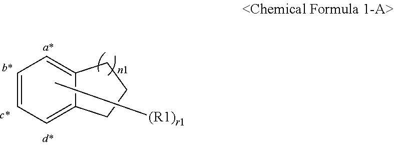

one to three of a* to d* are a position fused to or linked to Chemical Formula 1;

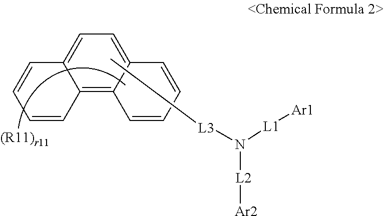

L1 to L3 are each independently a direct bond or a substituted or unsubstituted: arylene or divalent heterocyclic group; and
Ar1 and Ar2 are each independently a substituted or unsubstituted: aryl or heterocyclic group.

### CROSS-REFERENCE TO RELATED APPLICATIONS

This application is a National Stage Application of International Application No. PCT/KR2020/017321 filed on Nov. 30, 2020, which claims priority to and the benefit of Korean Patent Application No. 10-2019-0156836 filed in the Korean Intellectual Property Office on Nov. 29, 2019, the entire contents of which are incorporated herein by reference.

### TECHNICAL FIELD

The present specification relates to an organic light emitting device including: an anode; a cathode provided to face the anode; and an organic material layer between the anode and the cathode.

### BACKGROUND

In general, an organic light emitting phenomenon refers to a phenomenon in which electric energy is converted into light energy by using an organic material. An organic light emitting device using the organic light emitting phenomenon usually has a structure including an anode, a cathode, and an organic material layer interposed therebetween. Here, the organic material layer can have a multi-layered structure composed of different materials in many cases in order to improve the efficiency and stability of the organic light emitting device, and can be composed of, for example, a hole injection layer, a hole transport layer, a light emitting layer, an electron transport layer, an electron injection layer, and the like. In the structure of the organic light emitting device, if a voltage is applied between two electrodes, holes are injected from an anode into the organic material layer and electrons are injected from a cathode into the organic material layer, and when the injected holes and electrons meet each other, an exciton is formed, and light is emitted when the exciton falls down again to a ground state.

There is a continuous need for developing a new material for the aforementioned organic light emitting device.

### BRIEF DESCRIPTION

### Technical Problem

The present specification describes an organic light emitting device.

### Technical Solution

The present specification provides an organic light emitting device including: an anode; a cathode provided to face the anode; and an organic material layer between the anode and the cathode,

in which the organic material layer includes a light emitting layer and a first organic material layer provided between the anode and the light emitting layer,

the light emitting layer comprises a compound of the following Chemical Formula 1, and

the first organic material layer includes a compound of the following Chemical Formula 2:

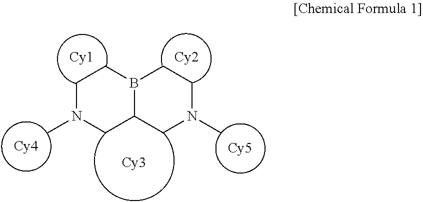

wherein in Chemical Formula 1:

Cy1 to Cy5 are the same as or different from each other, and are each independently one selected from the group consisting of a substituted or unsubstituted aromatic hydrocarbon ring, a substituted or unsubstituted aliphatic hydrocarbon ring, and a substituted or unsubstituted aromatic hetero ring, or a ring in which two or more rings selected from the above group are fused;

one or more of Cy1 to Cy5 are a ring of the following Chemical Formula 1-A;

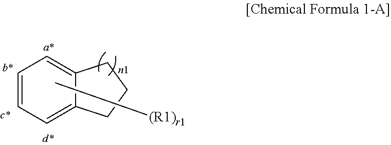

wherein in Chemical Formula 1-A:

one to three of a* to d* are a position that is fused to or linked to Chemical Formula 1;

R1 is hydrogen, deuterium, a cyano group, a halogen group, a substituted or unsubstituted alkyl group, a substituted or unsubstituted alkoxy group, a substituted or unsubstituted alkylthio group, a substituted or unsubstituted cycloalkyl group, a substituted or unsubstituted silyl group, a substituted or unsubstituted aryl group, a substituted or unsubstituted aryloxy group, a substituted or unsubstituted arylthio group, a substituted or unsubstituted heterocyclic group, or a substituted or unsubstituted amine group, or is bonded to an adjacent substituent to form a substituted or unsubstituted ring;

n1 is 1 or 2;

r1 is an integer from 0 to 11, and when r1 is 2 or higher, the R1s are the same as or different from each other;

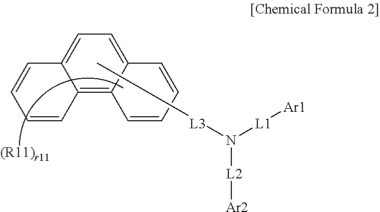

wherein in Chemical Formula 2:

L1 to L3 are the same as or different from each other, and are each independently a direct bond, a substituted or unsubstituted arylene group, or a substituted or unsubstituted divalent heterocyclic group;

Ar1 and Ar2 are the same as or different from each other, and are each independently a substituted or unsubstituted aryl group or a substituted or unsubstituted heterocyclic group;

R11 is hydrogen, deuterium, a cyano group, a halogen group, a substituted or unsubstituted alkyl group, a substituted or unsubstituted alkoxy group, a substituted or unsubstituted alkylthio group, a substituted or unsubstituted cycloalkyl group, a substituted or unsubstituted silyl group, a substituted or unsubstituted aryl group, a substituted or unsubstituted aryloxy group, a substituted or unsubstituted arylthio group, a substituted or unsubstituted heterocyclic group, or a substituted or unsubstituted amine group, or is bonded to an adjacent substituent to form a substituted or unsubstituted ring; and

r11 is an integer from 0 to 9, and when r11 is 2 or higher, the R11s are the same as or different from each other.

### Advantageous Effects

The organic light emitting device described in the present specification has a low driving voltage and has excellent efficiency characteristics and an excellent service life by including a compound of Chemical Formula 1 in a light emitting layer and including a compound of Chemical Formula 2 in a first organic material layer. Specifically, the low driving voltage, high efficiency, and service life can be improved by adjusting the appropriate HOMO energy level and LUMO energy level to adjust the transport degree of holes and/or electrons.

### BRIEF DESCRIPTION OF THE DRAWINGS

FIGS. 1, 2, 8, and 9 illustrate an example of an organic light emitting device according to an exemplary embodiment of the present specification.

FIGS. 3 to 7 illustrate an example of an organic light emitting device including two or more stacks.

### EXPLANATION OF REFERENCE NUMERALS AND SYMBOLS

1: Substrate/2: Anode/3: Hole injection layer/4: Hole transport layer/4a: First hole transport layer/4b: Second hole transport layer/4c: Third hole transport layer/4d: Fourth hole transport layer/4e: Fifth hole transport layer/4f: Sixth hole transport layer/4p: p-doped hole transport layer/4pa: p-doped first hole transport layer/4R: Red hole transport layer/4G: Green hole transport layer/4B: Blue hole transport layer/5: Electron blocking layer/6: Light emitting layer/6a: First light emitting layer/6b: Second light emitting layer/6c: Third light emitting layer/6BF: Blue fluorescent light emitting layer/6BFa: First blue fluorescent light emitting layer/6BFb: Second blue fluorescent light emitting layer/6BFc: Third blue fluorescent light emitting layer/6YGP: Yellow green phosphorescent light emitting layer/6RP: Red phosphorescent light emitting layer/6GP: Green phosphorescent light emitting layer/7: Hole blocking layer/8: Electron injection and transport layer/9: Electron transport layer/9a: First electron transport layer/9b: Second electron transport layer/9c: Third electron transport layer/10: Electron injection layer/11: Cathode/12: N-type charge generating layer/12a: First N-type charge generating layer/12b: Second N-type charge generating layer/13: P-type charge generating layer/13a: First P-type charge generating layer/13b: Second P-type charge generating layer/14: Capping layer

### DETAILED DESCRIPTION

Hereinafter, the present specification will be described in more detail.

The present specification provides an organic light emitting device including: an anode; a cathode provided to face the anode; and an organic material layer between the anode and the cathode,

in which the organic material layer includes a light emitting layer and a first organic material layer provided between the anode and the light emitting layer,

the light emitting layer includes the compound of Chemical Formula 1, and

the first organic material layer includes the compound of Chemical Formula 2.

Chemical Formula 1 of the present invention includes a fused ring of an aromatic hydrocarbon ring and an aliphatic hydrocarbon, which is represented by Chemical Formula 1-A. Chemical Formula 1 of the present invention has a low sublimation temperature by including Chemical Formula 1-A, and thus is highly stable, so that a device having excellent efficiency and long service life characteristics can be obtained when Chemical Formula 1 of the present invention is applied to the device.

Chemical Formula 2 of the present invention is an amine compound including a phenanthrenyl group, and is excellent in hole injection characteristics and characteristics of moving holes to the light emitting layer by including a tertiary amine group in which an aryl group is substituted in phenanthrene, and is excellent in stability to electrons, so that Chemical Formula 2 of the present invention increases the hole-electron binding probability by preventing excessive electron transfer from the light emitting layer. Accordingly, an organic light emitting device in which Chemical Formula 2 is used can exhibit high efficiency and long service life.

In this case, when the compound of Chemical Formula 1 is used in a light emitting layer and the compound of Chemical Formula 2 is used in a hole transport region, the stability of each material is excellent, so that the long service life characteristics of the device are enhanced, and the low voltage and high efficiency characteristics of the device are further enhanced by synergistic effects in the organic light emitting device.

When one part “includes” one constituent element in the present specification, unless otherwise specifically described, this does not mean that another constituent element is excluded, but means that another constituent element can be further included.

When one member is disposed “on” another member in the present specification, this includes not only a case where the one member is brought into contact with another member, but also a case where still another member is present between the two members.

In the present specification, * or a dotted line means a site bonded or fused to another substituent or a bonding portion.

In the present specification, Cn means that the number of carbon atoms is n, and Cn-Cm means that the number of carbon atoms is n to m.

Examples of the substituents in the present specification will be described below, but are not limited thereto.

The term “substitution” means that a hydrogen atom bonded to a carbon atom of a compound is changed into another substituent, and a position to be substituted is not limited as long as the position is a position at which the hydrogen atom is substituted, that is, a position at which the substituent can be substituted, and when two or more are substituted, the two or more substituents can be the same as or different from each other.

In the present specification, the term “substituted or unsubstituted” means being substituted with one or two or more substituents selected from the group consisting of deuterium, a halogen group, a cyano group (—CN), a silyl group, a boron group, an alkyl group, a cycloalkyl group, an aryl group, and a heterocyclic group, being substituted with a substituent to which two or more substituents among the exemplified substituents are linked, or having no substituent. For example, “the substituent to which two or more substituents are linked” can be a biphenyl group. That is, the biphenyl group can also be an aryl group, and can be interpreted as a substituent to which two phenyl groups are linked.

In an exemplary embodiment of the present specification, the “substituted or unsubstituted” means being substituted with one or more substituents selected from the group consisting of deuterium, a halogen group, a cyano group (—CN), a silyl group, a C1-C20 alkyl group, a C3-C60 cycloalkyl group, a C6-C60 aryl group, and a C2-C60 heterocyclic group, being substituted with a substituent to which two or more groups selected from the above group are linked, or having no substituent.

In an exemplary embodiment of the present specification, the “substituted or unsubstituted” means being substituted with one or more substituents selected from the group consisting of deuterium, a halogen group, a cyano group (—CN), a silyl group, a C1-C20 alkyl group, a C3-C60 cycloalkyl group, a C6-C60 aryl group, and a C2-C60 heterocyclic group, being substituted with a substituent to which two or more groups selected from the above group are linked, or having no substituent.

In an exemplary embodiment of the present specification, the “substituted or unsubstituted” means being substituted with one or more substituents selected from the group consisting of deuterium, a halogen group, a cyano group (—CN), a silyl group, a C1-C10 alkyl group, a C3-C30 cycloalkyl group, a C6-C30 aryl group, and a C2-C30 heterocyclic group, being substituted with a substituent to which two or more groups selected from the above group are linked, or having no substituent.

In an exemplary embodiment of the present specification, the “substituted or unsubstituted” means being substituted with one or more substituents selected from the group consisting of deuterium, a halogen group, a cyano group (—CN), a silyl group, a C1-C6 alkyl group, a C3-C20 cycloalkyl group, a C6-C20 aryl group, and a C2-C20 heterocyclic group, being substituted with a substituent to which two or more groups selected from the above group are linked, or having no substituent.

In the present specification, the fact that two or more substituents are linked indicates that hydrogen of any one substituent is replaced with another substituent. For example, an isopropyl group and a phenyl group can be linked to each other to become a substituent of

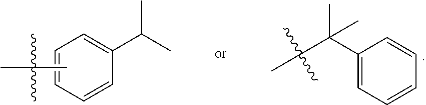

In the present specification, the fact that three substituents are linked to one another includes not only a case where (Substituent 1)-(Substituent 2)-(Substituent 3) are consecutively linked to one another, but also a case where (Substituent 2) and (Substituent 3) are linked to (Substituent 1). For example, two phenyl groups and an isopropyl group can be linked to each other to become a substituent of

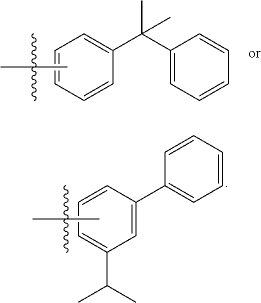

The same also applies to the case where four or more substituents are linked to one another.

In the present specification, “substituted with A or B” includes not only the case of being substituted with A alone or with B alone, but also the case of being substituted with A and B.

Examples of the substituents will be described below, but are not limited thereto.

In the present specification, examples of a halogen group include fluorine (F), chlorine (Cl), bromine (Br) or iodine (I).

In the present specification, a silyl group can be —SiY₁₁Y₁₂Y₁₃, and the Y₁₁, Y₁₂, and Y₁₃ can be each hydrogen, a substituted or unsubstituted alkyl group, or a substituted or unsubstituted aryl group. Specific examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, a boron group can be —BY₁₄Y₁₅, and Y₁₄ and Y₁₅ can be each hydrogen, a substituted or unsubstituted alkyl group, or a substituted or unsubstituted aryl group. Specific examples of the boron group include a trimethylboron group, a triethylboron group, a t-butyldimethylboron group, a triphenylboron group, a phenylboron group, and the like, but are not limited thereto.

In the present specification, an alkyl group can be straight-chained or branched, and the number of carbon atoms thereof is not particularly limited, but is preferably 1 to 60. According to an exemplary embodiment, the number of carbon atoms of the alkyl group is 1 to 30. According to another exemplary embodiment, the number of carbon atoms of the alkyl group is 1 to 20. According to still another exemplary embodiment, the number of carbon atoms of the alkyl group is 1 to 10. According to yet another exemplary embodiment, the number of carbon atoms of the alkyl group is 1 to 6. According to still yet another exemplary embodiment, the number of carbon atoms of the alkyl group is 1 to 4. Specific examples of the alkyl group include a methyl group, an ethyl group, a propyl group, an isopropyl group, a butyl group, an isobutyl group, a tert-butyl group, a pentyl group, a hexyl group, a heptyl group, an octyl group, and the like, but are not limited thereto.

In the present specification, an amine group can be selected from the group consisting of —NH₂, an alkylamine group, an alkylarylamine group, an arylamine group, an arylheteroarylamine group, an alkylheteroarylamine group, and a heteroarylamine group, and the number of carbon atoms thereof is not particularly limited, but is preferably 1 to 60. In the case of an arylamine group, the number of carbon atoms thereof is 6 to 60. According to another exemplary embodiment, the number of carbon atoms of the arylamine group is 6 to 40. Specific examples of the amine group include a methylamine group, a dimethylamine group, an ethylamine group, a diethylamine group, a phenylamine group, a naphthylamine group, a biphenylamine group, an anthracenylamine group, a 9-methylanthracenylamine group, a diphenylamine group, an N-phenylnaphthylamine group, a ditolylamine group, an N-phenyltolylamine group, a triphenylamine group, an N-phenyl-biphenylamine group, an N-phenylnaphthylamine group, an N-biphenylnaphthylamine group, an N-naphthyl-fluorenylamine group, an N-phenylphenanthrenyl-amine group, an N-biphenylphenanthrenylamine group, an N-phenylfluorenylamine group, an N-phenyl terphenylamine group, an N-phenanthrenylfluorenylamine group, an N-biphenylfluorenylamine group, an N-(4-(tert-butyl)-phenyl)-N-phenylamine group, an N,N-bis(4-(tert-butyl)-phenyl)amine group, an N,N-bis(3-(tert-butyl)phenyl)-amine group, and the like, but are not limited thereto.

In the present specification, an alkylarylamine group means an amine group in which an alkyl group and an aryl group are substituted with N of the amine group.

In the present specification, an arylheteroarylamine group means an amine group in which an aryl group and a heteroaryl group are substituted with N of the amine group.

In the present specification, an alkylheteroarylamine group means an amine group in which an alkyl group and a heteroaryl group are substituted with N of the amine group.

In the present specification, the alkyl group in the alkylamine group, the arylalkylamine group, the alkylthioxy group, the alkylsulfoxy group, and the alkylheteroarylamine group is the same as the above-described examples of the alkyl group. Specifically, examples of the alkylthioxy group include a methylthioxy group, an ethylthioxy group, a tert-butylthioxy group, a hexylthioxy group, an octylthioxy group, and the like, and examples of the alkylsulfoxy group include mesyl, an ethylsulfoxy group, a propylsulfoxy group, a butylsulfoxy group, and the like, but the examples are not limited thereto.

In the present specification, a cycloalkyl group is not particularly limited, but has preferably 3 to 60 carbon atoms, and according to an exemplary embodiment, the number of carbon atoms of the cycloalkyl group is 3 to 30. According to another exemplary embodiment, the number of carbon atoms of the cycloalkyl group is 3 to 20. According to still another exemplary embodiment, the number of carbon atoms of the cycloalkyl group is 3 to 6. The cycloalkyl group includes not only a single ring group, but also a double ring group such as a bridgehead, a fused ring, and a spiro ring. Specific examples thereof include a cyclopropyl group, a cyclobutyl group, a cyclopentyl group, a cyclohexyl group, a cycloheptyl group, a cyclooctyl group, an adamantyl group, and the like, but are not limited thereto.

In the present specification, cycloalkene is a ring group which has a double bond present in a hydrocarbon ring, but is a non-aromatic ring group, and the number of carbon atoms thereof is not particularly limited, but can be 3 to 60, and can be 3 to 30 according to an exemplary embodiment. The cycloalkene includes not only a single ring group, but also a double ring group such as a bridgehead, a fused ring, and a spiro ring. Examples of the cycloalkene include cyclopropene, cyclobutene, cyclopentene, cyclohexene, and the like, but are not limited thereto.

In the present specification, the alkoxy group is one in which an aryl group is linked to an oxygen atom, the alkylthio group is one in which an alkyl group is linked to a sulfur atom, and the above-described description on the alkyl group can be applied to the alkyl group of the alkoxy group and the alkylthio group.

In the present specification, an aryl group is not particularly limited, but has preferably 6 to 60 carbon atoms, and can be a monocyclic aryl group or a polycyclic aryl group. According to an exemplary embodiment, the number of carbon atoms of the aryl group is 6 to 30. According to an exemplary embodiment, the number of carbon atoms of the aryl group is 6 to 20. Examples of a monocyclic aryl group as the aryl group include a phenyl group, a biphenyl group, a terphenyl group, and the like, but are not limited thereto. Examples of the polycyclic aryl group include a naphthyl group, an anthracenyl group, a phenanthrenyl group, a pyrenyl group, a perylenyl group, a triphenylenyl group, a chrysenyl group, a fluorenyl group, and the like, but are not limited thereto.

In the present specification, No. 9 carbon atom (C) of a fluorenyl group can be substituted with an alkyl group, an aryl group, or the like, and two substituents can be bonded to each other to form a spiro structure such as cyclopentane or fluorene.

In the present specification, the substituted aryl group can also include a form in which an aliphatic ring is fused to the aryl group. For example, a tetrahydronaphthalene group or a dihydroindene group having the following structure is included in a substituted aryl group. In the following structure, one of the carbons of a benzene ring can be linked to another position.

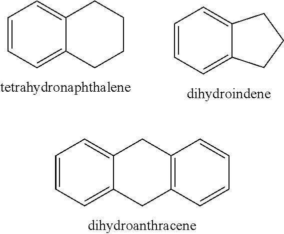

In the present specification, the alkylaryl group means an aryl group substituted with an alkyl group, and a substituent other than the alkyl group can be further linked.

In the present specification, an arylalkyl group means an alkyl group substituted with an aryl group, and a substituent other than the alkyl group can be further linked.

In the present specification, the aryloxy group is one in which an aryl group is linked to an oxygen atom, the arylthio group is one in which an aryl group is linked to a sulfur atom, and the above-described description on the aryl group can be applied to the aryl group of the aryloxy group and the arylthio group. An aryl group of an aryloxy group is the same as the above-described examples of the aryl group. Specifically, examples of the aryloxy group include a phenoxy group, a p-tolyloxy group, an m-tolyloxy group, a 3,5-dimethyl-phenoxy group, a 2,4,6-trimethylphenoxy group, a p-tert-butylphenoxy group, a 3-biphenyloxy group, a 4-biphenyloxy group, a 1-naphthyloxy group, a 2-naphthyloxy group, a 4-methyl-1-naphthyloxy group, a 5-methyl-2-naphthyloxy group, a 1-anthryloxy group, a 2-anthryloxy group, a 9-anthryloxy group, a 1-phenanthryloxy group, a 3-phenanthryloxy group, a 9-phenanthryloxy group, and the like, and examples of the arylthioxy group include a phenylthioxy group, a 2-methylphenylthioxy group, a 4-tert-butylphenylthioxy group, and the like, but the examples are not limited thereto.

In the present specification, a heterocyclic group is a cyclic group including one or more of N, O, P, S, Si, and Se as a heteroatom, and the number of carbon atoms thereof is not particularly limited, but is preferably 2 to 60. According to an exemplary embodiment, the number of carbon atoms of the heterocyclic group is 2 to 30. According to an exemplary embodiment, the number of carbon atoms of the heterocyclic group is 2 to 20. Examples of the heterocyclic group include a pyridyl group, a quinoline group, a thiophene group, a dibenzothiophene group, a furan group, a dibenzofuran group, a naphthobenzofuran group, a carbazole group, a benzocarbazole group, a naphthobenzothiophene group, a dibenzosilole group, a naphthobenzosilole group, a hexahydrocarbazole group, dihydroacridine group, a dihydrodibenzoazasiline group, a phenoxazine group, a phenothiazine group, a dihydrodibenzoazasiline group, a spiro(dibenzosilole-dibenzoazasiline) group, a spiro(acridine-fluorene) group, and the like, but are not limited thereto:

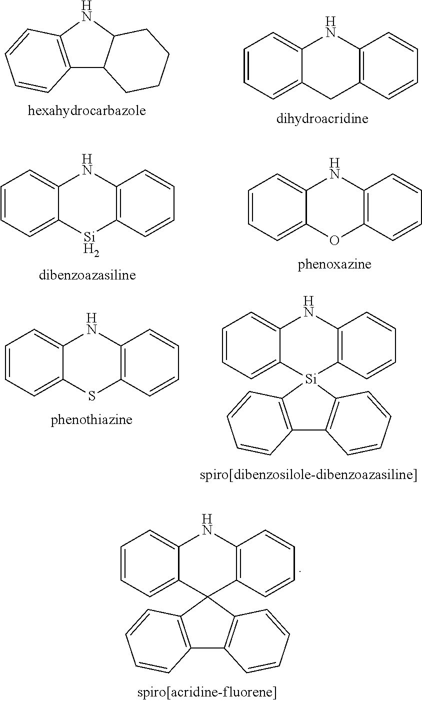

In the present specification, the above-described description on the heterocyclic group can be applied to a heteroaryl group except for an aromatic heteroaryl group.

In the present specification, an aromatic hydrocarbon ring means a hydrocarbon ring in which pi electrons are completely conjugated and are planar, and the description on the aryl group can be applied to an aromatic hydrocarbon ring except for a divalent aromatic hydrocarbon ring.

In the present specification, an aliphatic hydrocarbon ring has a cyclically bonded structure, and means a non-aromatic ring. Examples of the aliphatic hydrocarbon ring include cycloalkane or cycloalkene, and the above-described description on the cycloalkyl group or cycloalkenyl group can be applied to the aliphatic hydrocarbon ring except for a divalent aliphatic hydrocarbon ring. Further, a substituted aliphatic hydrocarbon ring also includes an aliphatic hydrocarbon ring in which aromatic rings are fused.

In the present specification, a fused ring of an aromatic hydrocarbon ring and an aliphatic hydrocarbon ring means that an aromatic hydrocarbon ring and an aliphatic hydrocarbon ring form a fused ring. Examples of the fused ring of the aromatic ring and the aliphatic ring include a 1,2,3,4-tetrahydronaphthalene group, a 2,3-dihydro-1H-indene group, and the like, but are not limited thereto.

In the present specification, the “adjacent” group can mean a substituent substituted with an atom directly linked to an atom in which the corresponding substituent is substituted, a substituent disposed to be sterically closest to the corresponding substituent, or another substituent substituted with an atom in which the corresponding substituent is substituted. For example, two substituents substituted at the ortho position in a benzene ring and two substituents substituted with the same carbon in an aliphatic ring can be interpreted as groups which are “adjacent” to each other. In addition, substituents (four in total) linked to two consecutive carbons in an aliphatic ring can be interpreted as “adjacent” groups.

In the present specification, the “adjacent groups are bonded to each other to form a ring” among the substituents means that a substituent is bonded to an adjacent group to form a substituted or unsubstituted hydrocarbon ring or a substituted or unsubstituted hetero ring.

In the present specification, “a five-membered or six-membered ring formed by bonding adjacent groups” means that a ring including a substituent participating in the ring formation is five-membered or six-membered. It is possible to include an additional ring fused to the ring including the substituent participating in the ring formation.

In the present specification, the above-described description on the aryl group can be applied to an arylene group except for a divalent arylene group.

Hereinafter, Chemical Formula 1 will be described in detail.

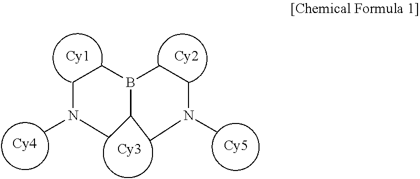

In Chemical Formula 1:

Cy1 to Cy5 are the same as or different from each other, and are each independently one selected from the group consisting of a substituted or unsubstituted aromatic hydrocarbon ring, a substituted or unsubstituted aliphatic hydrocarbon ring, and a substituted or unsubstituted aromatic hetero ring, or a ring in which two or more rings selected from the above group are fused;

one or more of Cy1 to Cy5 are a ring of the following Chemical Formula 1-A:

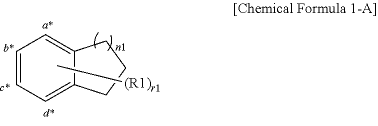

wherein in Chemical Formula 1-A:

one to three of a* to d* are a position that is fused to or linked to Chemical Formula 1;

R1 is hydrogen, deuterium, a cyano group, a halogen group, a substituted or unsubstituted alkyl group, a substituted or unsubstituted alkoxy group, a substituted or unsubstituted alkylthio group, a substituted or unsubstituted cycloalkyl group, a substituted or unsubstituted silyl group, a substituted or unsubstituted aryl group, a substituted or unsubstituted aryloxy group, a substituted or unsubstituted arylthio group, a substituted or unsubstituted heterocyclic group, or a substituted or unsubstituted amine group, or is bonded to an adjacent substituent to form a substituted or unsubstituted ring,

n1 is 1 or 2; and

r1 is an integer from 0 to 11, and when r1 is 2 or higher, R1's are the same as or different from each other.

In an exemplary embodiment of the present specification, Cy1 to Cy5 are the same as or different from each other, and are each independently one selected from the group consisting of a substituted or unsubstituted C6-C60 aromatic hydrocarbon ring, a substituted or unsubstituted C5-C60 aliphatic hydrocarbon ring, and a substituted or unsubstituted C2-C60 aromatic hetero ring, or a C9-C60 ring in which two or more rings selected from the above group are fused.

In an exemplary embodiment of the present specification, Cy1 to Cy5 are the same as or different from each other, and are each independently one selected from the group consisting of a substituted or unsubstituted C6-C30 aromatic hydrocarbon ring, a substituted or unsubstituted C5-C30 aliphatic hydrocarbon ring, and a substituted or unsubstituted C2-C30 aromatic hetero ring, or a C9-C30 ring in which two or more rings selected from the above group are fused.

In an exemplary embodiment of the present specification, Cy1 to Cy5 are the same as or different from each other, and are each independently one selected from the group consisting of a substituted or unsubstituted C6-C20 aromatic hydrocarbon ring, a substituted or unsubstituted C5-C20 aliphatic hydrocarbon ring, and a substituted or unsubstituted C2-C20 aromatic hetero ring, or a C9-C20 ring in which two or more rings selected from the above group are fused.

In an exemplary embodiment of the present specification, Cy1 to Cy5 are the same as or different from each other, and are each independently one selected from the group consisting of a substituted or unsubstituted benzene ring, a substituted or unsubstituted naphthalene ring, a substituted or unsubstituted fluorene ring, a substituted or unsubstituted cyclohexene ring, a substituted or unsubstituted cyclopentene ring, a substituted or unsubstituted dibenzofuran ring, a substituted or unsubstituted dibenzothiophene ring, a substituted or unsubstituted dibenzosilole ring, a substituted or unsubstituted naphthobenzofuran ring, a substituted or unsubstituted naphthobenzothiophene ring, a substituted or unsubstituted naphthobenzosilole ring, a substituted or unsubstituted tetrahydronaphthobenzofuran ring, a substituted or unsubstituted tetrahydronaphtho-benzothiophene ring, or a substituted or unsubstituted tetrahydronaphthobenzosilole ring, or a ring in which two or more rings selected from the above group are fused.

In an exemplary embodiment of the present specification, Cy1 to Cy5 are the same as or different from each other, and are each independently a substituted or unsubstituted benzene ring, a substituted or unsubstituted naphthalene ring, a substituted or unsubstituted fluorene ring, a substituted or unsubstituted tetrahydronaphthalene ring, a substituted or unsubstituted dihydroindene ring, a substituted or unsubstituted dihydroanthracene ring, a substituted or unsubstituted dibenzofuran ring, a substituted or unsubstituted dibenzothiophene ring, a substituted or unsubstituted dibenzosilole ring, a substituted or unsubstituted naphthobenzofuran ring, a substituted or unsubstituted naphthobenzo-thiophene ring, a substituted or unsubstituted naphthobenzosilole ring, a substituted or unsubstituted tetrahydronaphthobenzofuran ring, a substituted or unsubstituted tetrahydronaphthobenzothiophene ring, or a substituted or unsubstituted tetrahydronaphtho-benzosilole ring.

In an exemplary embodiment of the present specification, Cy1 to Cy5 are the same as or different from each other, and are each independently a substituted or unsubstituted benzene ring, a substituted or unsubstituted naphthalene ring, a substituted or unsubstituted fluorene ring, a substituted or unsubstituted dibenzofuran ring, a substituted or unsubstituted dibenzothiophene ring, a substituted or unsubstituted dibenzosilole ring, a substituted or unsubstituted naphthobenzofuran ring, a substituted or unsubstituted naphthobenzothiophene ring, a substituted or unsubstituted naphthobenzosilole ring, a substituted or unsubstituted tetrahydronaphtho-benzofuran ring, a substituted or unsubstituted tetrahydronaphthobenzothiophene ring, a substituted or unsubstituted tetrahydronaphthobenzosilole ring, or the ring of Chemical Formula 1-A.

In an exemplary embodiment of the present specification, Cy1 to Cy3 are the same as or different from each other, and are each independently one selected from the group consisting of a substituted or unsubstituted C6-C20 aromatic hydrocarbon ring, a substituted or unsubstituted C5-C20 aliphatic hydrocarbon ring, and a substituted or unsubstituted C2-C30 aromatic hetero ring, or a C9-C20 ring in which two or more rings selected from the above group are fused.

In an exemplary embodiment of the present specification, Cy1 to Cy3 are the same as or different from each other, and are each independently a substituted or unsubstituted benzene ring, a substituted or unsubstituted tetrahydronaphthalene ring, a substituted or unsubstituted dihydroindene ring, a substituted or unsubstituted dihydroanthracene ring, a substituted or unsubstituted dibenzofuran ring, a substituted or unsubstituted dibenzothiophene ring, a substituted or unsubstituted naphthobenzofuran ring, a substituted or unsubstituted naphthobenzo-thiophene ring, a substituted or unsubstituted tetrahydronaphthobenzofuran ring, or a substituted or unsubstituted tetrahydronaphthobenzothiophene ring.

In an exemplary embodiment of the present specification, Cy1 to Cy3 are the same as or different from each other, and are each independently a substituted or unsubstituted benzene ring, a substituted or unsubstituted dibenzofuran ring, a substituted or unsubstituted dibenzothiophene ring, a substituted or unsubstituted tetrahydronaphthobenzofuran ring, a substituted or unsubstituted tetrahydronaphthobenzothiophene ring, or the ring of Chemical Formula 1-A.

In an exemplary embodiment of the present specification, Cy1 and Cy2 are the same as or different from each other, and are each independently a substituted or unsubstituted benzene ring, a substituted or unsubstituted dibenzofuran ring, a substituted or unsubstituted dibenzothiophene ring, a substituted or unsubstituted tetrahydronaphthobenzofuran ring, a substituted or unsubstituted tetrahydronaphthobenzo-thiophene ring, or the ring of Chemical Formula 1-A.

In an exemplary embodiment of the present specification, Cy3 is a substituted or unsubstituted benzene ring.

In an exemplary embodiment of the present specification, Cy4 and Cy5 are the same as or different from each other, and are each independently one selected from the group consisting of a substituted or unsubstituted C6-C20 aromatic hydrocarbon ring, a substituted or unsubstituted C5-C20 aliphatic hydrocarbon ring, and a substituted or unsubstituted C2-C20 aromatic hetero ring, or a C9-C20 ring in which two or more rings selected from the above group are fused.

In an exemplary embodiment of the present specification, Cy4 and Cy5 are the same as or different from each other, and are each independently a substituted or unsubstituted benzene ring, a substituted or unsubstituted naphthalene ring, a substituted or unsubstituted fluorene ring, a substituted or unsubstituted tetrahydronaphthalene ring, a substituted or unsubstituted dihydroindene ring, a substituted or unsubstituted dihydroanthracene ring, a substituted or unsubstituted dibenzofuran ring, a substituted or unsubstituted dibenzothiophene ring, a substituted or unsubstituted dibenzosilole ring, a substituted or unsubstituted naphthobenzofuran ring, a substituted or unsubstituted naphthobenzo-thiophene ring, or a substituted or unsubstituted naphthobenzosilole ring.

In an exemplary embodiment of the present specification, Cy4 and Cy5 are the same as or different from each other, and are each independently a substituted or unsubstituted benzene ring, a substituted or unsubstituted naphthalene ring, a substituted or unsubstituted fluorene ring, a substituted or unsubstituted tetrahydronaphthalene ring, a substituted or unsubstituted dihydroindene ring, a substituted or unsubstituted dihydroanthracene ring, a substituted or unsubstituted dibenzofuran ring, a substituted or unsubstituted dibenzothiophene ring, a substituted or unsubstituted dibenzosilole ring, a substituted or unsubstituted naphthobenzofuran ring, a substituted or unsubstituted naphthobenzo-thiophene ring, a substituted or unsubstituted naphthobenzosilole ring, or the ring of Chemical Formula 1-A.

In an exemplary embodiment of the present specification, Cy1 to Cy5 are each unsubstituted or substituted with a substituent of R1 to R6 to be described below.

In an exemplary embodiment of the present specification, one or more, two or more, three or more, or four or more of Cy1 to Cy5 are the ring of Chemical Formula 1-A.

In an exemplary embodiment of the present specification, one or more, two or more, three or more, or all of Cy1, Cy2, Cy4, and Cy5 are the ring of Chemical Formula 1-A.

In an exemplary embodiment of the present specification, when Cy1 or Cy2 is the ring of Chemical Formula 1-A, adjacent two of a* to d* are positions which are fused to Chemical Formula 1. Specifically, a* and b*, b* and c*, or c* and d* are positions which are fused to Chemical Formula 1.

In an exemplary embodiment of the present specification, when Cy3 is the ring of Chemical Formula 1-A, adjacent three of a* to d* are positions which are fused to Chemical Formula 1. Specifically, a* to c*, or b* to d* are positions which are fused to Chemical Formula 1.

In an exemplary embodiment of the present specification, when Cy4 or Cy5 is the ring of Chemical Formula 1-A, one of a* to d* is a position which is linked to Chemical Formula 1. Specifically, a*, b*, c*, or d* is a position which is linked to Chemical Formula 1.

In an exemplary embodiment of the present specification, Cy4 and Cy5 are the same as or different from each other, and are each independently Chemical Formula 1-A, or the following Chemical Formula 1-B.

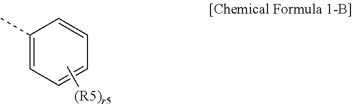

In Chemical Formula 1-B:

a dotted line is a position which is linked to Chemical Formula 1;

R5 is hydrogen, deuterium, a cyano group, a halogen group, a substituted or unsubstituted alkyl group, a substituted or unsubstituted alkoxy group, a substituted or unsubstituted alkylthio group, a substituted or unsubstituted cycloalkyl group, a substituted or unsubstituted silyl group, a substituted or unsubstituted aryl group, a substituted or unsubstituted aryloxy group, a substituted or unsubstituted arylthio group, a substituted or unsubstituted heterocyclic group, a substituted or unsubstituted amine group, or Chemical Formula 1-A, or is bonded to an adjacent substituent to form a substituted or unsubstituted ring; and

r5 is an integer from 0 to 5, and when r5 is 2 or higher, the R5s are the same as or different from each other.

In an exemplary embodiment of the present specification, a substituent (R5) instead of hydrogen is linked to the ortho position with respect to the dotted line of Chemical Formula 1-B.

In an exemplary embodiment of the present specification, Chemical Formula 1-B is the following Chemical Formula 1-B-1 or 1-B-2:

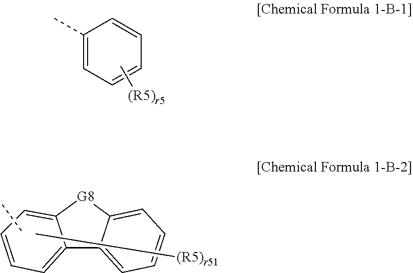

wherein in Chemical Formulae 1-B-1 and 1-B-2:

the dotted line, R5, and r5 are the same as those defined in Chemical Formula 1-B;

G8 is —O—, —S—,—NG9-, —CG9G10-, or —SiG9G10-; and

r51 is an integer from 0 to 7, and when r51 is 2 or higher, R5's are the same as or different from each other.

In the present specification, adjacent groups of R5's are each independently bonded to each other to form any one ring of the following structures:

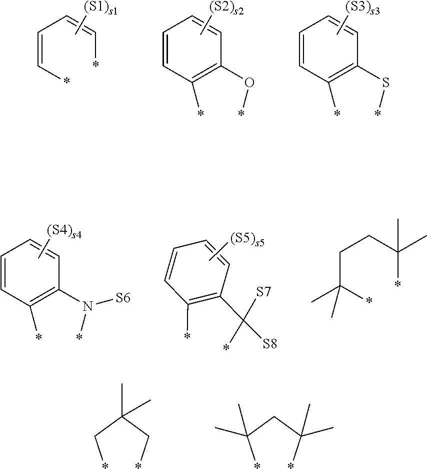

wherein in the structures, S1 to S8 are each independently hydrogen, deuterium, a halogen group, a cyano group, a substituted or unsubstituted alkyl group, a substituted or unsubstituted cycloalkyl group, a substituted or unsubstituted aryl group, or a substituted or unsubstituted heterocyclic group;

s1 to s5 are each an integer from 0 to 4;

when s1 is 2 or higher, the S1s are the same as or different from each other;

when s2 is 2 or higher, the S2s are the same as or different from each other;

when s3 is 2 or higher, the S3s are the same as or different from each other;

when s4 is 2 or higher, the S4s are the same as or different from each other;

when s5 is 2 or higher, the S5s are the same as or different from each other; and

* denotes a position that is substituted.

In an exemplary embodiment of the present specification, S6 to S8, G9, and G10 are the same as or different from each other, and are each independently hydrogen, deuterium, a substituted or unsubstituted alkyl group, or a substituted or unsubstituted aryl group, or are bonded to an adjacent substituent to form a substituted or unsubstituted ring.

In an exemplary embodiment of the present specification, S6 to S8, G9, and G10 are the same as or different from each other, and are each independently hydrogen, deuterium, a substituted or unsubstituted C1-C10 alkyl group, or a substituted or unsubstituted C6-C30 aryl group, or are bonded to an adjacent substituent to form a substituted or unsubstituted C6-C30 aromatic hydrocarbon ring.

In an exemplary embodiment of the present specification, S6 to S8, G9, and G10 are the same as or different from each other, and are each independently hydrogen; deuterium; a C1-C6 alkyl group which is unsubstituted or substituted with deuterium; or a C6-C30 aryl group which is unsubstituted or substituted with deuterium or a C1-C6 alkyl group, or are bonded to an adjacent substituent to form a C6-C20 aromatic hydrocarbon ring.

In an exemplary embodiment of the present specification, S6 to S8, G9, and G10 are the same as or different from each other, and are each independently hydrogen, deuterium, a methyl group, or a phenyl group, or are bonded to each other to form a fluorene ring.

In an exemplary embodiment of the present specification, Chemical Formula 1-B-2 is selected from the following structures:

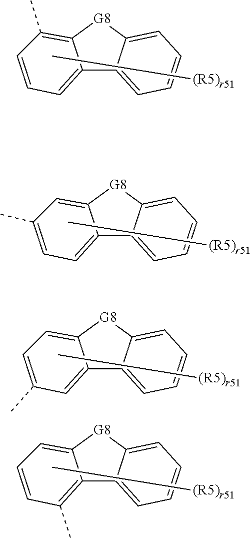

wherein in the structures, the dotted line, G8, R5, and r51 are the same as those defined in Chemical Formula 1-B-2.

In an exemplary embodiment of the present specification, R1 is hydrogen, deuterium, a cyano group, a halogen group, a substituted or unsubstituted alkyl group, a substituted or unsubstituted alkoxy group, a substituted or unsubstituted alkylthio group, a substituted or unsubstituted cycloalkyl group, a substituted or unsubstituted silyl group, a substituted or unsubstituted aryl group, a substituted or unsubstituted aryloxy group, a substituted or unsubstituted arylthio group, a substituted or unsubstituted heterocyclic group, or a substituted or unsubstituted amine group, or is bonded to an adjacent substituent to form a substituted or unsubstituted ring.

In an exemplary embodiment of the present specification, R1 is hydrogen, deuterium, a cyano group, a halogen group, a substituted or unsubstituted C1-C6 alkyl group, a substituted or unsubstituted C1-C18 alkylsilyl group, a substituted or unsubstituted C6-C60 arylsilyl group, or a substituted or unsubstituted C6-C20 aryl group, or is bonded to an adjacent substituent to form a substituted or substituted C6-C20 aromatic hydrocarbon ring.

In an exemplary embodiment of the present specification, R1 is hydrogen, deuterium, a cyano group, a halogen group, a C1-C10 alkyl group, a C1-C10 alkoxy group, a C1-C10 alkylthio group, a silyl group, a C6-C30 aryl group, a C6-C30 aryloxy group, a C6-C30 arylthio group, a C2-C30 heterocyclic group, or an amine group, or is bonded to an adjacent substituent to form a C6-C30 ring, and the substituent or ring is unsubstituted or substituted with one or more substituents selected from the group consisting of deuterium, a cyano group, a halogen group, a C1-C10 alkyl group, a silyl group, and a C6-C30 aryl group or a substituent to which two or more groups selected from the above group are linked.

In an exemplary embodiment of the present specification, R1 is hydrogen; deuterium; a cyano group; a halogen group; a C1-C6 alkyl group which is unsubstituted or substituted with one or more substituents selected from the group consisting of deuterium, a cyano group, a halogen group, a C1-C6 alkyl group, a silyl group, and a C6-C20 aryl group or a substituent to which two or more groups selected from the above group are linked; a C1-C18 alkylsilyl group which is unsubstituted or substituted with one or more substituents selected from the group consisting of deuterium, a cyano group, a halogen group, a C1-C6 alkyl group, a silyl group, and a C6-C20 aryl group or a substituent to which two or more groups selected from the above group are linked; a C6-C60 arylsilyl group which is unsubstituted or substituted with one or more substituents selected from the group consisting of deuterium, a cyano group, a halogen group, a C1-C6 alkyl group, a silyl group, and a C6-C20 aryl group or a substituent to which two or more groups selected from the above group are linked; or a C6-C20 aryl group which is unsubstituted or substituted with one or more substituents selected from the group consisting of deuterium, a cyano group, a halogen group, a C1-C6 alkyl group, a silyl group, and a C6-C20 aryl group or a substituent to which two or more groups selected from the above group are linked, or is bonded to an adjacent substituent to form a C6-C20 aromatic hydrocarbon ring which is unsubstituted or substituted with one or more substituents selected from the group consisting of deuterium, a cyano group, a halogen group, a C1-C6 alkyl group, a silyl group, and a C6-C20 aryl group or a substituent to which two or more groups selected from the above group are linked.

In an exemplary embodiment of the present specification, R1 is hydrogen; deuterium; a C1-C6 alkyl group; or a C6-C20 aryl group which is unsubstituted or substituted with deuterium, a cyano group, a halogen group, a C1-C6 alkyl group, or a C1-C18 trialkylsilyl group, or is bonded to adjacent R1 to form a C6-C20 aromatic hydrocarbon ring.

In an exemplary embodiment of the present specification, R1 is hydrogen, deuterium, a substituted or unsubstituted methyl group, a substituted or unsubstituted phenyl group, a substituted or unsubstituted biphenyl group, or a substituted or unsubstituted naphthyl group, or is bonded to adjacent R1 to from a substituted or unsubstituted benzene ring.

In an exemplary embodiment of the present specification, R1 is hydrogen; deuterium; a methyl group; a phenyl group which is unsubstituted or substituted with deuterium, a cyano group, a halogen group, a methyl group, a tert-butyl group, or a trimethylsilyl group; a biphenyl group which is unsubstituted or substituted with deuterium, a cyano group, a halogen group, a methyl group, a tert-butyl group, or a trimethylsilyl group; or a naphthyl group which is unsubstituted or substituted with deuterium, a cyano group, a halogen group, a methyl group, a tert-butyl group, or a trimethylsilyl group, or is bonded to adjacent R1 to form a benzene ring.

In an exemplary embodiment of the present specification, r1 is 2 or higher.

In an exemplary embodiment of the present specification, two or four of the R1s are a methyl group.

In an exemplary embodiment of the present specification, Chemical Formula 1-A is any one of the following Chemical Formulae 1-A-1 to 1-A-3:

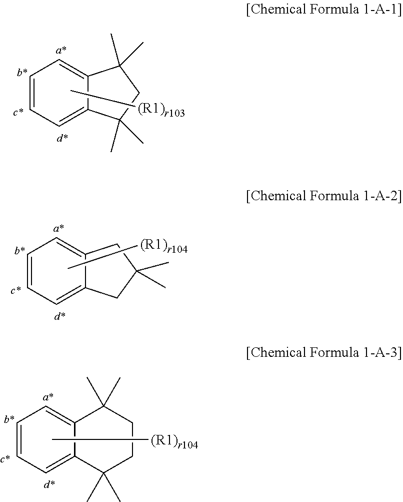

wherein in Chemical Formulae 1-A-1 to 1-A-3:

a* to d* and R1 are the same as those defined in Chemical Formula 1-A;

r103 is an integer from 0 to 5, and r104 is an integer from 0 to 7; and

when r103 and r104 are each 2 or higher, the R1s are the same as or different from each other.

In an exemplary embodiment of the present specification, Chemical Formula 1-A-3 is any one of the following Chemical Formula 1-A-3-1 or 1-A-3-2:

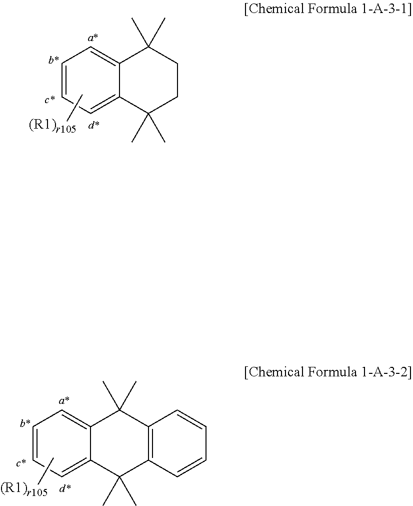

wherein in Chemical Formulae 1-A-3-1 and 1-A-3-2:

a* to d* and R1 are the same as those defined in Chemical Formula 1-A; and

r105 is an integer from 0 to 3, and when r105 is 2 or higher, R1's are the same as or different from each other.

In an exemplary embodiment of the present specification, r103 is 0 or 1.

In an exemplary embodiment of the present specification, r104 is 0 or 1.

In an exemplary embodiment of the present specification, r105 is 0 or 1.

In an exemplary embodiment of the present specification, Cy1 or Cy2 is Chemical Formula 1-A.

In an exemplary embodiment of the present specification, Cy4 or Cy5 is Chemical Formula 1-A.

In an exemplary embodiment of the present specification, Chemical Formula 1 is the following Chemical Formula 101 or 102:

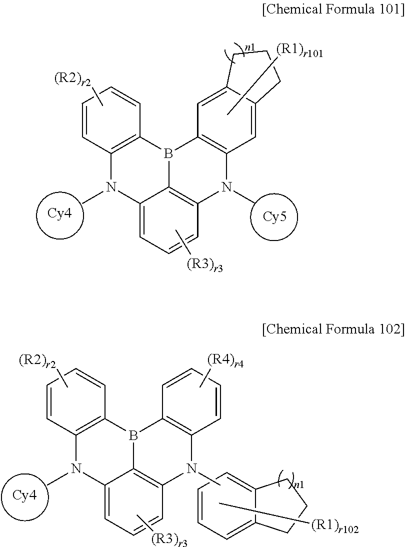

wherein in Chemical Formulae 101 and 102:

Cy4, Cy5, R1, and n1 are the same as those defined in Chemical Formula 1;

R2 to R4 are the same as or different from each other, and are each independently hydrogen, deuterium, a cyano group, a halogen group, a substituted or unsubstituted alkyl group, a substituted or unsubstituted alkoxy group, a substituted or unsubstituted alkylthio group, a substituted or unsubstituted cycloalkyl group, a substituted or unsubstituted silyl group, a substituted or unsubstituted aryl group, a substituted or unsubstituted aryloxy group, a substituted or unsubstituted arylthio group, a substituted or unsubstituted heterocyclic group, or a substituted or unsubstituted amine group, or are bonded to an adjacent substituent to form a substituted or unsubstituted ring;

r101 is an integer from 0 to 10, r102 is an integer from 0 to 11, r2 and r4 are an integer from 0 to 4, and r3 is an integer from 0 to 3; and

when r101, r102, and r2 to r4 are each 2 or higher, substituents in the parenthesis are the same as or different from each other.

In an exemplary embodiment of the present specification, Chemical Formula 1 is any one of the following Chemical Formulae 111 to 118:

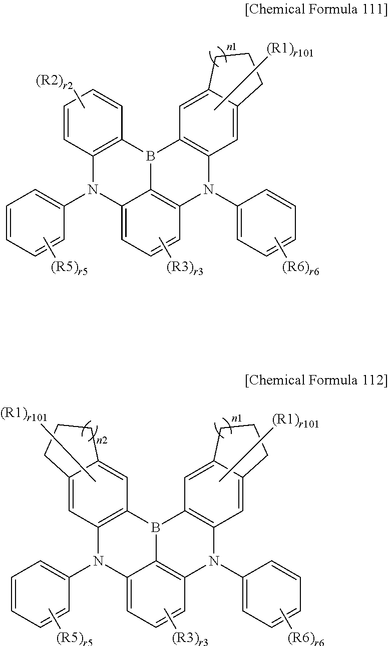

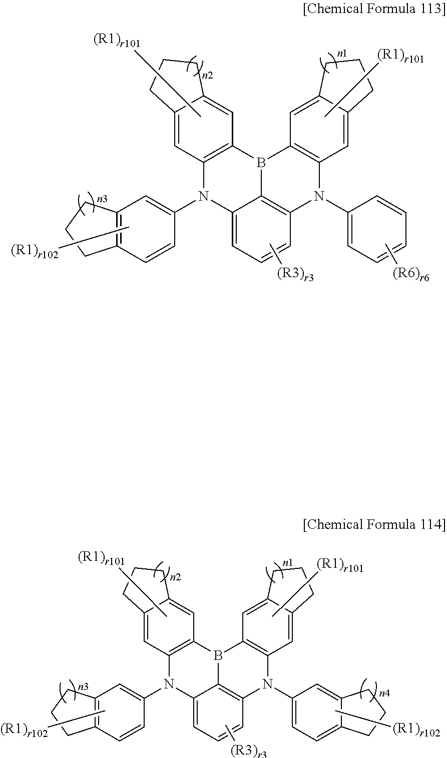

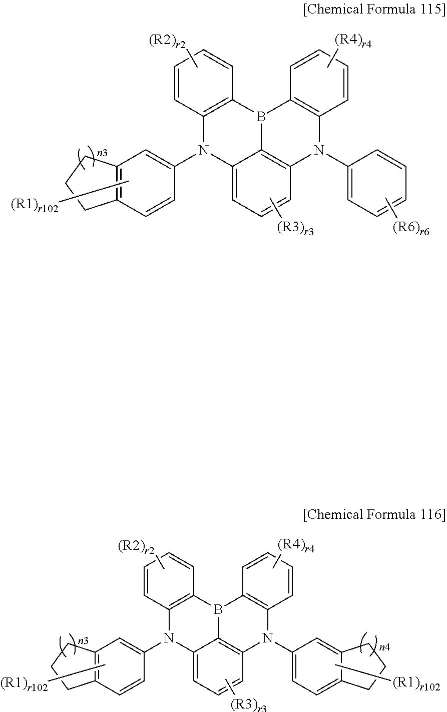

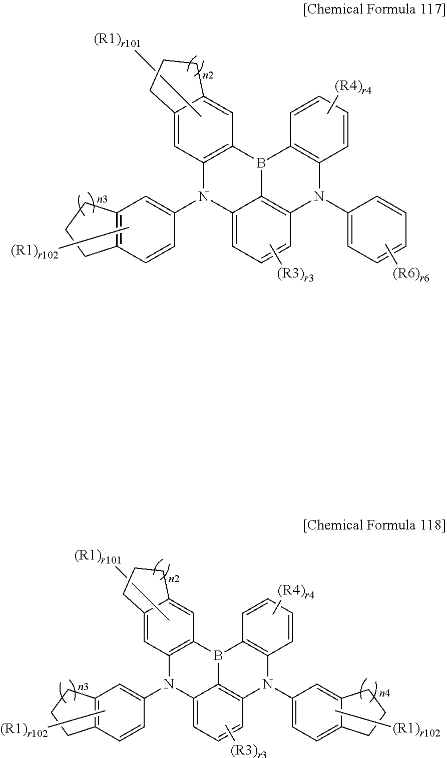

wherein in Chemical Formulae 111 to 118:

R1 to R6 are the same as or different from each other, and are each independently hydrogen, deuterium, a cyano group, a halogen group, a substituted or unsubstituted alkyl group, a substituted or unsubstituted alkoxy group, a substituted or unsubstituted alkylthio group, a substituted or unsubstituted cycloalkyl group, a substituted or unsubstituted silyl group, a substituted or unsubstituted aryl group, a substituted or unsubstituted aryloxy group, a substituted or unsubstituted arylthio group, a substituted or unsubstituted heterocyclic group, or a substituted or unsubstituted amine group, or are bonded to an adjacent substituent to form a substituted or unsubstituted ring,

n1 to n4 are each 1 or 2;

r2 and r4 are an integer from 0 to 4, r3 is an integer from 0 to 3, r5 and r6 are an integer from 0 to 5, r101 is an integer from 0 to 10, and r102 is an integer from 0 to 11; and

when r2 to r6, r101, and r102 are each 2 or higher, substituents in the parenthesis are the same as or different from each other.

In an exemplary embodiment of the present specification, the following rings of Chemical Formulae 111 to 118 are the same as or different from each other, and are each independently any one of Chemical Formulae 1-A-1 to 1-A-3:

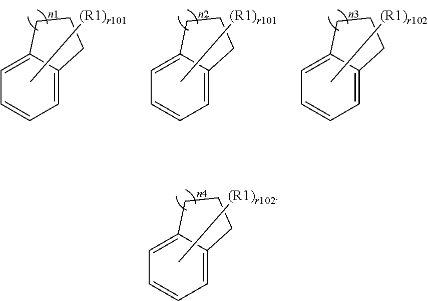

In an exemplary embodiment of the present specification, R2 to R6 are hydrogen, deuterium, a cyano group, a halogen group, a substituted or unsubstituted C1-C10 alkyl group, a substituted or unsubstituted C1-C10 alkoxy group, a substituted or unsubstituted C1-C10 alkylthio group, a substituted or unsubstituted C3-C30 cycloalkyl group, a substituted or unsubstituted silyl group, a substituted or unsubstituted C6-C30 aryl group, a substituted or unsubstituted C6-C30 aryloxy group, a substituted or unsubstituted C6-C30 arylthio group, a substituted or unsubstituted C2-C30 heterocyclic group, or a substituted or unsubstituted amine group, or are bonded to an adjacent substituent to form a substituted or unsubstituted C6-C30 ring.

In an exemplary embodiment of the present specification, R2 to R6 are hydrogen, deuterium, a cyano group, a halogen group, a substituted or unsubstituted C1-C10 alkyl group, a substituted or unsubstituted C1-C10 alkoxy group, a substituted or unsubstituted C1-C10 alkylthio group, a substituted or unsubstituted C3-C30 cycloalkyl group, a substituted or unsubstituted C1-C30 alkylsilyl group, a substituted or unsubstituted C6-C90 arylsilyl group, a substituted or unsubstituted C6-C30 aryl group, a substituted or unsubstituted C6-C30 aryloxy group, a substituted or unsubstituted C6-C30 arylthio group, a substituted or unsubstituted C2-C30 heterocyclic group, a substituted or unsubstituted C6-C60 arylamine group, or a substituted or unsubstituted C2-C60 heteroarylamine group, or are bonded to an adjacent substituent to form a substituted or unsubstituted C6-C30 ring.

In an exemplary embodiment of the present specification, R2 to R6 are the same as or different from each other, and are each independently hydrogen, deuterium, a cyano group, a halogen group, a substituted or unsubstituted alkyl group, a substituted or unsubstituted cycloalkyl group, a substituted or unsubstituted silyl group, a substituted or unsubstituted aryl group, a substituted or unsubstituted heterocyclic group, or a substituted or unsubstituted amine group.

In an exemplary embodiment of the present specification, R2 to R6 are the same as or different from each other, and are each independently hydrogen, deuterium, a cyano group, a halogen group, a substituted or unsubstituted C1-C10 alkyl group, a substituted or unsubstituted C3-C30 cycloalkyl group, a substituted or unsubstituted C1-C30 alkylsilyl group, a substituted or unsubstituted C6-C90 arylsilyl group, a substituted or unsubstituted C6-C30 aryl group, a substituted or unsubstituted C2-C30 heterocyclic group, or a substituted or unsubstituted C6-C60 arylamine group.

In an exemplary embodiment of the present specification, R2 to R6 are the same as or different from each other, and are each independently hydrogen, deuterium, a cyano group, a halogen group, C1-C10 alkyl group, C3-C30 cycloalkyl group, a C1-C30 alkylsilyl group, a C6-C90 arylsilyl group, a C6-C30 aryl group, a fused ring group of a C6-C30 aromatic hydrocarbon ring and an aliphatic hydrocarbon ring, a C2-C30 heterocyclic group, or a C6-C60 arylamine group, and R2 to R6 are unsubstituted or substituted with one or more substituents selected from the group consisting of deuterium, a cyano group, a halogen group, a C1-C10 alkyl group, a silyl group, and a C6-C30 aryl group or a substituent to which two or more groups selected from the above group are linked.

In an exemplary embodiment of the present specification, R5 and R6 are bonded to an adjacent substituent to form a substituted or unsubstituted hydrocarbon ring or a substituted or unsubstituted hetero ring.

The hetero rings of R2 to R6 are N, O, or S-containing hetero rings.

In an exemplary embodiment of the present specification, R2 to R6 are the same as or different from each other, and can be each independently any one of the following structures, or a substituted or unsubstituted carbazolyl group. Specifically, R2 to R4 are the same as or different from each other, and can be each independently any one of the following structures, or a substituted or unsubstituted carbazolyl group, and preferably, R3's are the same as or different from each other, and can be each independently any one of the following structures, or a substituted or unsubstituted carbazolyl group:

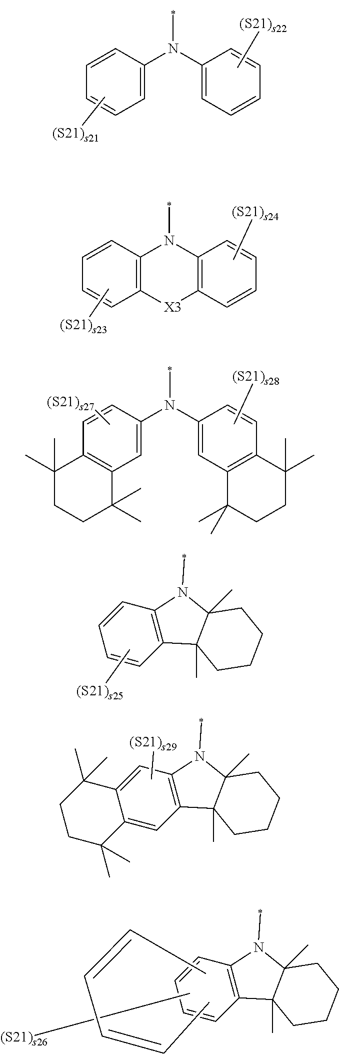

wherein in the above structures, the S21s are each independently hydrogen, deuterium, a halogen group, a cyano group, a substituted or unsubstituted alkyl group, a substituted or unsubstituted cycloalkyl group, a substituted or unsubstituted aryl group, or a substituted or unsubstituted heterocyclic group;

X3 is O, S, or CR″R′″;

R″ and R″′ are the same as or different from each other, and are each independently a substituted or unsubstituted alkyl group, a substituted or unsubstituted cycloalkyl group, a substituted or unsubstituted aryl group, or a substituted or unsubstituted heterocyclic group;

s21 and s22 are each an integer from 0 to 5;

s23 to s25 are each an integer from 0 to 4;

s26 is an integer from 0 to 6;

s27 and s28 are an integer from 0 to 3;

s29 is an integer from 0 to 2;

when s21 to s28 are each 2 or higher, the S21s are the same as or different from each other;

when s29 is 2, the S21s are the same as or different from each other; and

* denotes a position that is substituted.

In an exemplary embodiment of the present specification, the N-containing hetero ring of R2 to R6 is the following Chemical Formula HAr1 or HAr2:

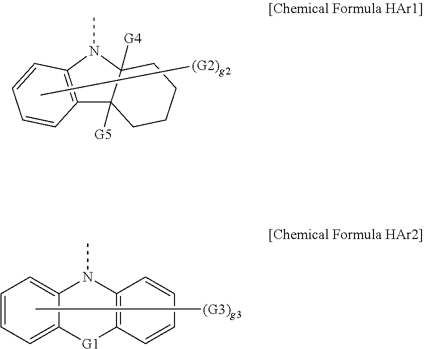

wherein in Chemical Formulae HAr1 and HAr2:

a dotted line is a position which is linked to Chemical Formula 1;

G1 is a direct bond, —O—, —S—,—CG6G7-, or -SiG6G7-;

G2 to G7 are the same as or different from each other, and are each independently hydrogen, deuterium, a cyano group, a halogen group, a substituted or unsubstituted alkyl group, a substituted or unsubstituted alkoxy group, a substituted or unsubstituted alkylthio group, a substituted or unsubstituted cycloalkyl group, a substituted or unsubstituted silyl group, a substituted or unsubstituted aryl group, a substituted or unsubstituted aryloxy group, a substituted or unsubstituted arylthio group, a substituted or unsubstituted heterocyclic group, or a substituted or unsubstituted amine group, or are bonded to an adjacent substituent to form a substituted or unsubstituted ring;

g2 is an integer from 0 to 12, and g3 is an integer from 0 to 8; and

when g2 and g3 are each 2 or higher, substituents in the parenthesis are the same as or different from each other.

In an exemplary embodiment of the present specification, G1 is a direct bond, —O—, —S—, or —CG6G7-.

In an exemplary embodiment of the present specification, G2 to G5 are the same as or different from each other, and are each independently hydrogen, deuterium, a substituted or unsubstituted alkyl group, a substituted or unsubstituted silyl group, or a substituted or unsubstituted aryl group, or are bonded to an adjacent substituent to form a substituted or unsubstituted ring.

In an exemplary embodiment of the present specification, G2 to G5 are the same as or different from each other, and are each independently hydrogen, deuterium, a substituted or unsubstituted C1-C10 alkyl group, a substituted or unsubstituted C1-C30 alkylsilyl group, a substituted or unsubstituted C6-C90 arylsilyl group, or a substituted or unsubstituted C6-C30 aryl group, or are bonded to an adjacent substituent to form a substituted or unsubstituted C6-C30 aromatic hydrocarbon ring.

In an exemplary embodiment of the present specification, G2 to G5 are the same as or different from each other, and are each independently hydrogen; deuterium; a C1-C6 alkyl group which is unsubstituted or substituted with deuterium; a C1-C18 alkylsilyl group; a C6-C60 arylsilyl group; or a C6-C30 aryl group which is unsubstituted or substituted with deuterium or a C1-C6 alkyl group, or are bonded to an adjacent substituent to form a C6-C20 aromatic hydrocarbon ring.

In an exemplary embodiment of the present specification, G2 and G3 are the same as or different from each other, and are each independently hydrogen, deuterium, a substituted or unsubstituted methyl group, a substituted or unsubstituted tert-butyl group, a substituted or unsubstituted trimethylsilyl group, or a substituted or unsubstituted phenyl group, or are bonded to an adjacent substituent to form a substituted or unsubstituted benzene ring.

In an exemplary embodiment of the present specification, G2 and G3 are the same as or different from each other, and are each independently hydrogen; deuterium; a methyl group; a tert-butyl group; a trimethylsilyl group; or a phenyl group which is unsubstituted or substituted with a methyl group or a tert-butyl group, or are bonded to an adjacent substituent to form a benzene ring.

In an exemplary embodiment of the present specification, G4 and G5 are a methyl group.

In an exemplary embodiment of the present specification, G6 and G7 are the same as or different from each other, and are each independently hydrogen, deuterium, a substituted or unsubstituted alkyl group, or a substituted or unsubstituted aryl group, or are bonded to an adjacent substituent to form a substituted or unsubstituted ring.

In an exemplary embodiment of the present specification, G6 and G7 are the same as or different from each other, and are each independently hydrogen, deuterium, a substituted or unsubstituted C1-C10 alkyl group, or a substituted or unsubstituted C6-C30 aryl group, or are bonded to an adjacent substituent to form a substituted or unsubstituted C6-C30 aromatic hydrocarbon ring.

In an exemplary embodiment of the present specification, G6 and G7 are the same as or different from each other, and are each independently hydrogen; deuterium; a C1-C6 alkyl group which is unsubstituted or substituted with deuterium; or a C6-C30 aryl group which is unsubstituted or substituted with deuterium or a C1-C6 alkyl group, or are bonded to an adjacent substituent to form a C6-C20 aromatic hydrocarbon ring.

In an exemplary embodiment of the present specification, G6 and G7 are the same as or different from each other, and are each independently hydrogen, deuterium, a methyl group, or a phenyl group, or are bonded to each other to form a fluorene ring.

In an exemplary embodiment of the present specification, R2 and R4 are the same as or different from each other, and are each independently hydrogen, deuterium, a substituted or unsubstituted methyl group, a substituted or unsubstituted ethyl group, a substituted or unsubstituted isopropyl group, a substituted or unsubstituted tert-butyl group, a substituted or unsubstituted cyclohexyl group, a substituted or unsubstituted adamantyl group, a substituted or unsubstituted trimethylsilyl group, a substituted or unsubstituted hexahydrocarbazole group, a substituted or unsubstituted phenoxazine group, a substituted or unsubstituted phenothiazine group, a substituted or unsubstituted dihydroacridine group, a substituted or unsubstituted carbazole group, or a substituted or unsubstituted diphenylamine group.

In an exemplary embodiment of the present specification, R2 and R4 are the same as or different from each other, and are each independently hydrogen, deuterium, a substituted or unsubstituted methyl group, a substituted or unsubstituted ethyl group, a substituted or unsubstituted isopropyl group, a substituted or unsubstituted tert-butyl group, a substituted or unsubstituted cyclohexyl group, a substituted or unsubstituted adamantyl group, a substituted or unsubstituted trimethylsilyl group, Chemical Formula HAr1, Chemical Formula HAr2, or a substituted or unsubstituted diphenylamine group.

In an exemplary embodiment of the present specification, R2 and R4 are the same as or different from each other, and are each independently hydrogen; deuterium; a methyl group which is unsubstituted or substituted with deuterium or a phenyl group; an ethyl group; an isopropyl group which is unsubstituted or substituted with a phenyl group; a tert-butyl group; a cyclohexyl group; an adamantyl group; a trimethylsilyl group; a hexahydrocarbazole group which is unsubstituted or substituted with a methyl group, a tert-butyl group, a phenyl group, or a trimethylsilyl group; a phenoxazine group which is unsubstituted or substituted with a methyl group or a tert-butyl group; a phenothiazine group which is unsubstituted or substituted with a methyl group or a tert-butyl group; a dihydroacridine group which is unsubstituted or substituted with a methyl group or a tert-butyl group; a carbazole group which is unsubstituted or substituted with a methyl group, a tert-butyl group, a phenyl group, or a trimethylsilyl group; or a diphenylamine group which is unsubstituted or substituted with a methyl group, a tert-butyl group, a phenyl group, or a trimethylsilyl group, and which is unfused or fused with a cyclohexene ring or a cyclopentene ring.

In an exemplary embodiment of the present specification, R3 is hydrogen, deuterium, a substituted or unsubstituted methyl group, a substituted or unsubstituted ethyl group, a substituted or unsubstituted isopropyl group, a substituted or unsubstituted tert-butyl group, a substituted or unsubstituted cyclohexyl group, a substituted or unsubstituted adamantyl group, a substituted or unsubstituted phenyl group, a substituted or unsubstituted biphenyl group, a substituted or unsubstituted naphthyl group, a substituted or unsubstituted fluorenyl group, a substituted or unsubstituted hexahydrocarbazole group, a substituted or unsubstituted hexahydrobenzocarbazole group, a substituted or unsubstituted phenoxazine group, a substituted or unsubstituted phenothiazine group, a substituted or unsubstituted dihydroacridine group, a substituted or unsubstituted carbazole group, a substituted or unsubstituted dibenzofuran group, a substituted or unsubstituted dibenzothiophene group, or a substituted or unsubstituted diphenylamine group.

In an exemplary embodiment of the present specification, R3 is hydrogen, deuterium, a substituted or unsubstituted methyl group, a substituted or unsubstituted ethyl group, a substituted or unsubstituted isopropyl group, a substituted or unsubstituted tert-butyl group, a substituted or unsubstituted cyclohexyl group, a substituted or unsubstituted adamantyl group, a substituted or unsubstituted phenyl group, a substituted or unsubstituted biphenyl group, a substituted or unsubstituted naphthyl group, a substituted or unsubstituted fluorenyl group, Chemical Formula HAr1, Chemical Formula HAr2, a substituted or unsubstituted dibenzofuran group, a substituted or unsubstituted dibenzothiophene group, a substituted or unsubstituted diphenylamine group, or a substituted or unsubstituted phenylbiphenylamine group.

In an exemplary embodiment of the present specification, R3 is hydrogen; deuterium; a methyl group which is unsubstituted or substituted with deuterium; an ethyl group; an isopropyl group; a tert-butyl group; a cyclohexyl group; an adamantyl group; a phenyl which is unsubstituted or substituted with deuterium, a methyl group, or a tert-butyl group; a biphenyl group; a naphthyl group; a fluorenyl group which is unsubstituted or substituted with a methyl group or a phenyl group; a hexahydrocarbazole group which is unsubstituted or substituted with deuterium, a methyl group, a tert-butyl group, a phenyl group, a tolyl group, a xylyl group, a tert-butylphenyl group, or a trimethylsilyl group; a hexahydrobenzocarbazole group; a phenoxazine group which is unsubstituted or substituted with a methyl group or a tert-butyl group; a phenothiazine group which is unsubstituted or substituted with a methyl group or a tert-butyl group; a dihydroacridine group which is unsubstituted or substituted with a methyl group or a phenyl group; a carbazole group which is unsubstituted or substituted with a methyl group or a tert-butyl group; a dibenzofuran group; a dibenzothiophene group; or a diphenylamine group which is unsubstituted or substituted with a methyl group, a tert-butyl group, a phenyl group, or a trimethylsilyl group, and which is unfused or fused with a cyclohexene ring or a cyclopentene ring.

In an exemplary embodiment of the present specification, R5 and R6 are the same as or different from each other, and are each independently hydrogen, deuterium, a cyano group, a halogen group, a substituted or unsubstituted C1-C10 alkyl group, a substituted or unsubstituted C1-C10 alkoxy group, a substituted or unsubstituted C1-C10 alkylthio group, a substituted or unsubstituted C3-C30 cycloalkyl group, a substituted or unsubstituted silyl group, a substituted or unsubstituted C6-C30 aryl group, a substituted or unsubstituted C6-C30 aryloxy group, a substituted or unsubstituted C6-C30 arylthio group, a substituted or unsubstituted C2-C30 heterocyclic group, or a substituted or unsubstituted amine group, or are bonded to an adjacent substituent to form a substituted or unsubstituted C2-C30 ring.

In an exemplary embodiment of the present specification, R5 and R6 are the same as or different from each other, and are each independently hydrogen; deuterium; a cyano group; a halogen group; a C1-C6 alkyl group which is unsubstituted or substituted with deuterium or a C6-C20 aryl group; a C3-C20 cycloalkyl group; a C1-C18 alkylsilyl group; a C6-C60 arylsilyl group; a C6-C20 aryl group which is unsubstituted or substituted with one or more substituents selected from the group consisting of deuterium, a cyano group, a halogen group, a C1-C6 alkyl group, a silyl group, and a C6-C20 aryl group or a substituent to which two or more groups selected from the above group are linked; a C2-C20 heterocyclic group which is unsubstituted or substituted with one or more substituents selected from the group consisting of deuterium, a cyano group, a halogen group, a C1-C6 alkyl group, a silyl group, and a C6-C20 aryl group or a substituent to which two or more groups selected from the above group are linked; or a C6-C40 arylamine group which is unsubstituted or substituted with one or more substituents selected from the group consisting of deuterium, a cyano group, a halogen group, a C1-C6 alkyl group, a silyl group, and a C6-C20 aryl group or a substituent to which two or more groups selected from the above group are linked, and which is unfused or fused with cyclohexene or cyclopentene, or are bonded to an adjacent substituent to form a C2-C25 ring which is unsubstituted or substituted with one or more substituents selected from the group consisting of deuterium, a cyano group, a halogen group, a C1-C6 alkyl group, a silyl group, and a C6-C20 aryl group or a substituent to which two or more groups selected from the above group are linked.

In an exemplary embodiment of the present specification, R5 and R6 are the same as or different from each other, and are each independently hydrogen, deuterium, a cyano group, a halogen group, a substituted or unsubstituted methyl group, a substituted or unsubstituted ethyl group, a substituted or unsubstituted isopropyl group, a substituted or unsubstituted tert-butyl group, a substituted or unsubstituted cyclohexyl group, a substituted or unsubstituted adamantyl group, a substituted or unsubstituted trimethylsilyl group, a substituted or unsubstituted tert-butyldimethylsilyl group, a substituted or unsubstituted phenyl group, a substituted or unsubstituted biphenyl group, a substituted or unsubstituted terphenyl group, or a substituted or unsubstituted naphthyl group.

In an exemplary embodiment of the present specification, R5 and R6 are the same as or different from each other, and are each independently hydrogen; deuterium; a cyano group; a halogen group; a methyl group which is unsubstituted or substituted with deuterium, a halogen group, or a phenyl group; an ethyl group; an isopropyl group which is unsubstituted or substituted with a phenyl group; a tert-butyl group; a cyclohexyl group; an adamantyl group; a trimethylsilyl group; a tert-butyldimethylsilyl group; a phenyl group which is unsubstituted or substituted with deuterium, a cyano group, a halogen group, a methyl group, an ethyl group, an isopropyl group, a tert-butyl group, a 2-methyl-2-phenylpropyl group, a trifluoromethyl group, a cyclohexyl group, an adamantyl group, a trimethylsilyl group, a tert-butyldimethylsilyl group, a phenyl group, or a naphthyl group; a biphenyl group which is unsubstituted or substituted with deuterium, a cyano group, a halogen group, a methyl group, an ethyl group, an isopropyl group, a tert-butyl group, a 2-methyl-2-phenylpropyl group, a trifluoromethyl group, a cyclohexyl group, an adamantyl group, a trimethylsilyl group, a tert-butyldimethylsilyl group, a phenyl group, or a naphthyl group; a substituted or unsubstituted terphenyl group; or a naphthyl group.

In an exemplary embodiment of the present specification, R5 is bonded to adjacent R5 to form a substituted or unsubstituted ring, and R6 is bonded to adjacent R6 to form a substituted or unsubstituted ring.

In an exemplary embodiment of the present specification, a ring formed by bonding R5 or R6 to an adjacent substituent is an indene ring, a spiro[fluorene-indene]ring, an indole ring, a benzofuran ring, a benzothiophene ring, a benzosilole ring, a benzoindole ring, a naphthofuran ring, a naphthothiophene ring, or a naphthosilole ring, and the ring is unsubstituted or substituted with the above-described substituent.

In an exemplary embodiment of the present specification, R5 and R6 are bonded to an adjacent substituent to form an indene ring, a spiro[fluorene-indene]ring, an indole ring, a benzofuran ring, a benzothiophene ring, a benzosilole ring, a benzoindole ring, a naphthofuran ring, a naphthothiophene ring, or a naphthosilole ring, and the ring is unsubstituted or substituted with a C1-C6 alkyl group or a C6-C20 aryl group.

In an exemplary embodiment of the present specification, R5 and R6 are bonded to an adjacent substituent to form an indene ring which is unsubstituted or substituted with a methyl group or a phenyl group; a spiro[fluorene-indene]ring; an indole ring which is unsubstituted or substituted with a phenyl group; a benzofuran group which is unsubstituted or substituted with a methyl group, a tert-butyl group, or a phenyl group; a benzothiophene ring which is unsubstituted or substituted with a methyl group, a tert-butyl group, or a phenyl group; a benzosilole ring which is unsubstituted or substituted with a methyl group or a phenyl group; or a naphthofuran ring.

In an exemplary embodiment of the present specification, Chemical Formula 1 is one selected from the following compounds:

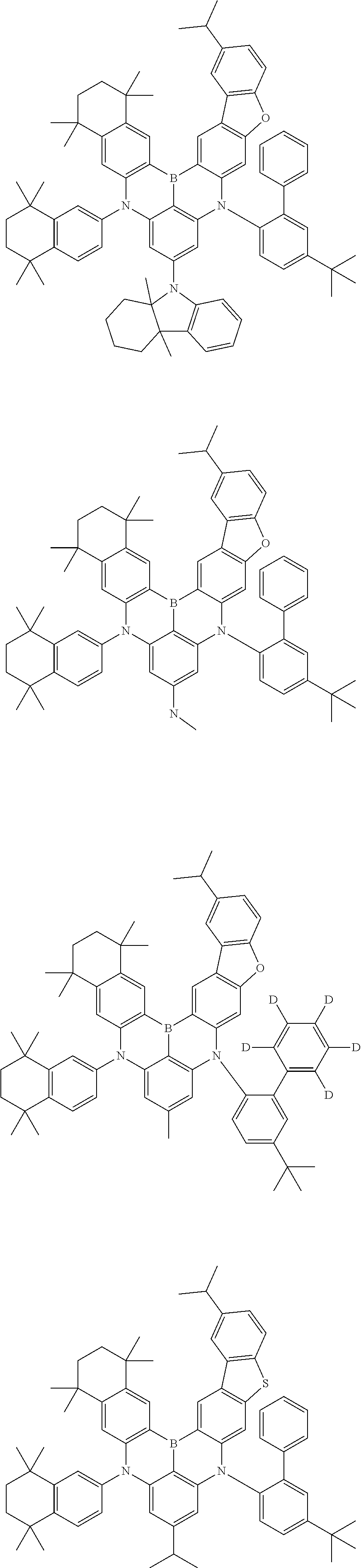

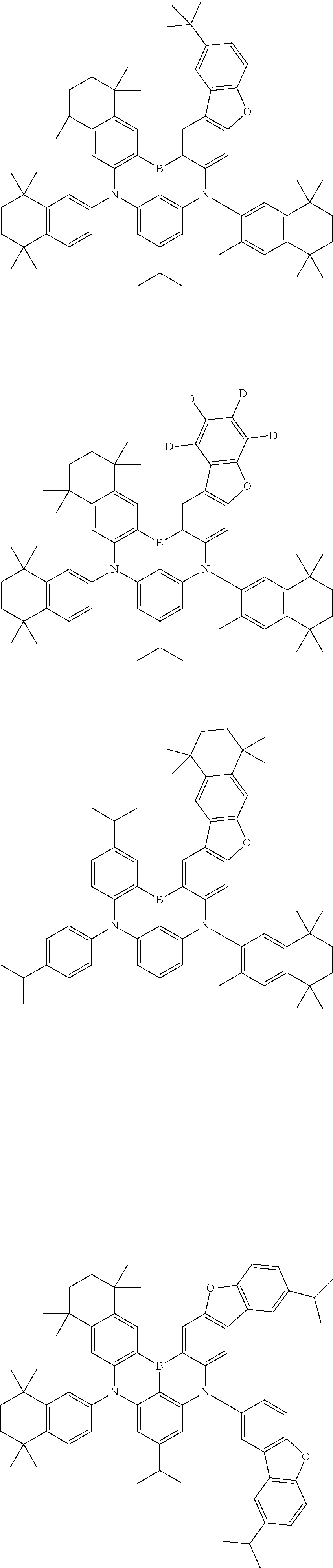

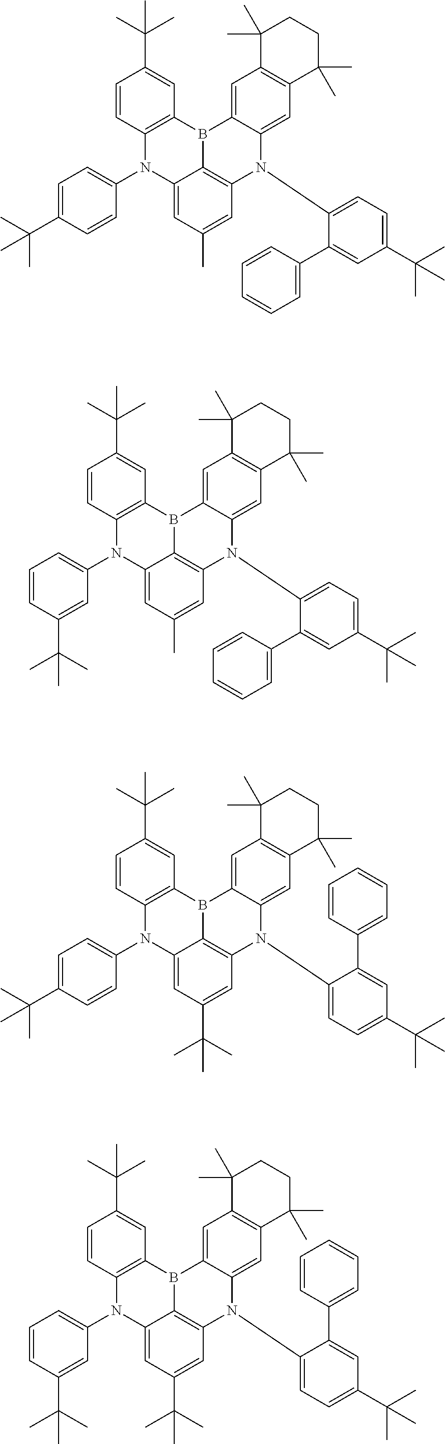

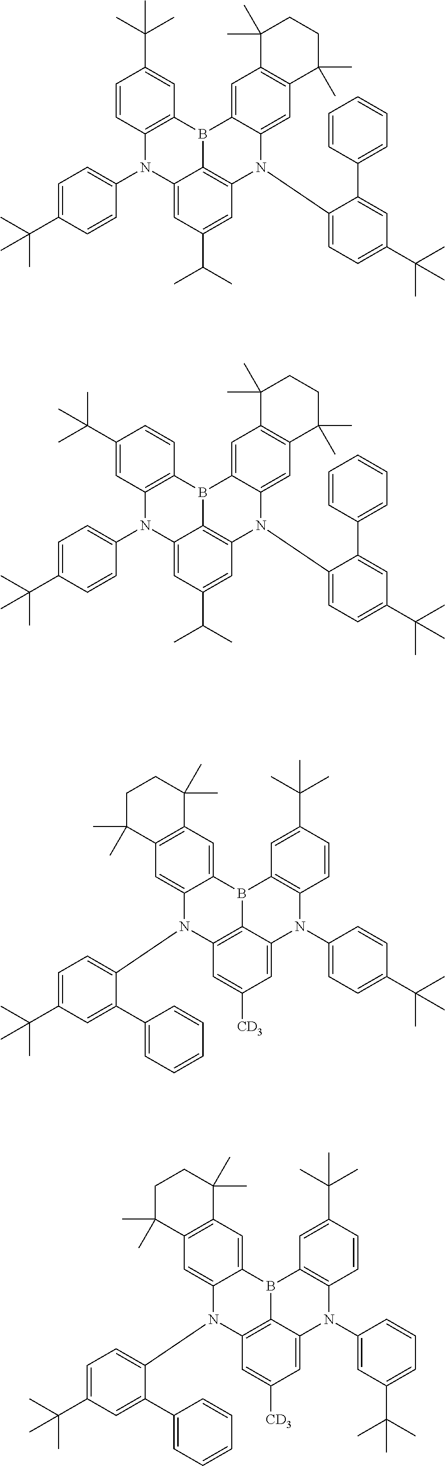

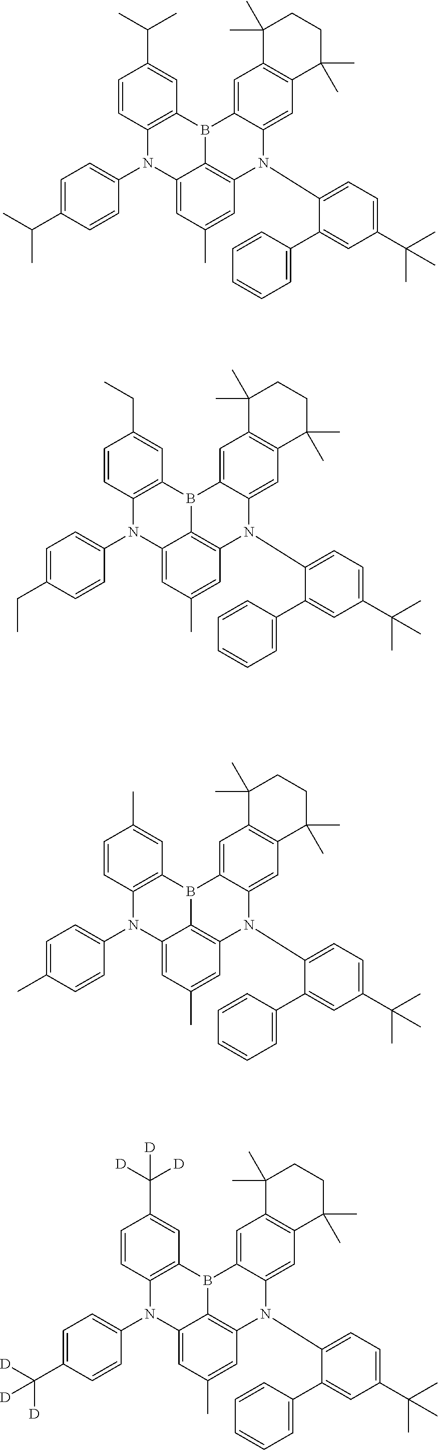

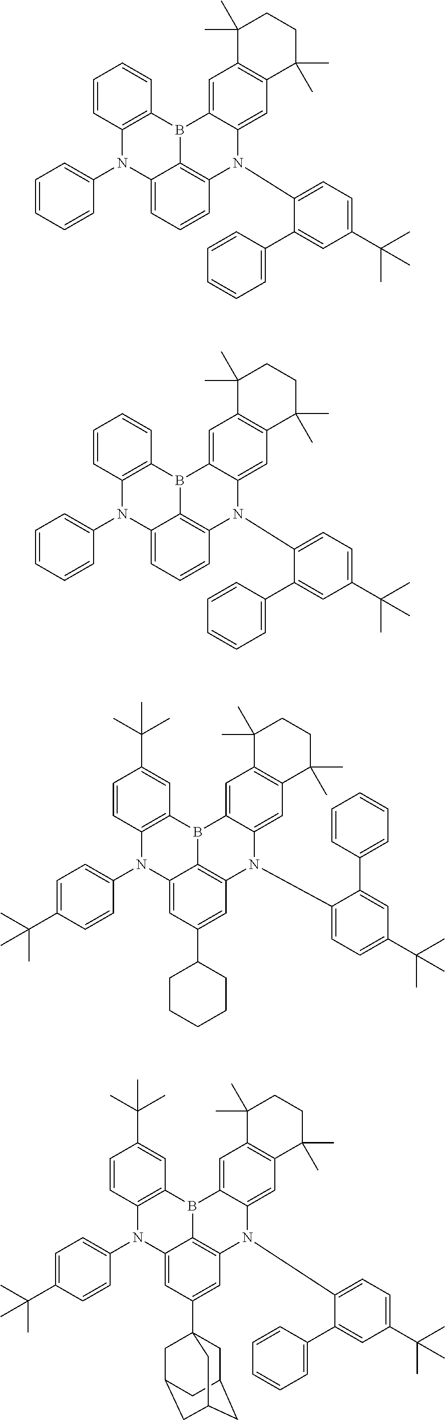

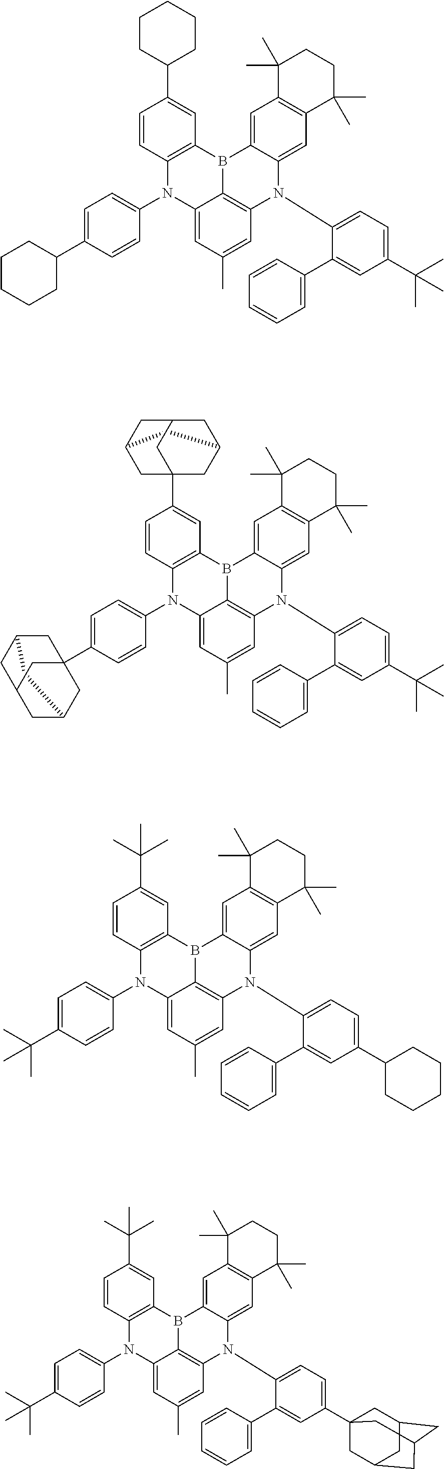

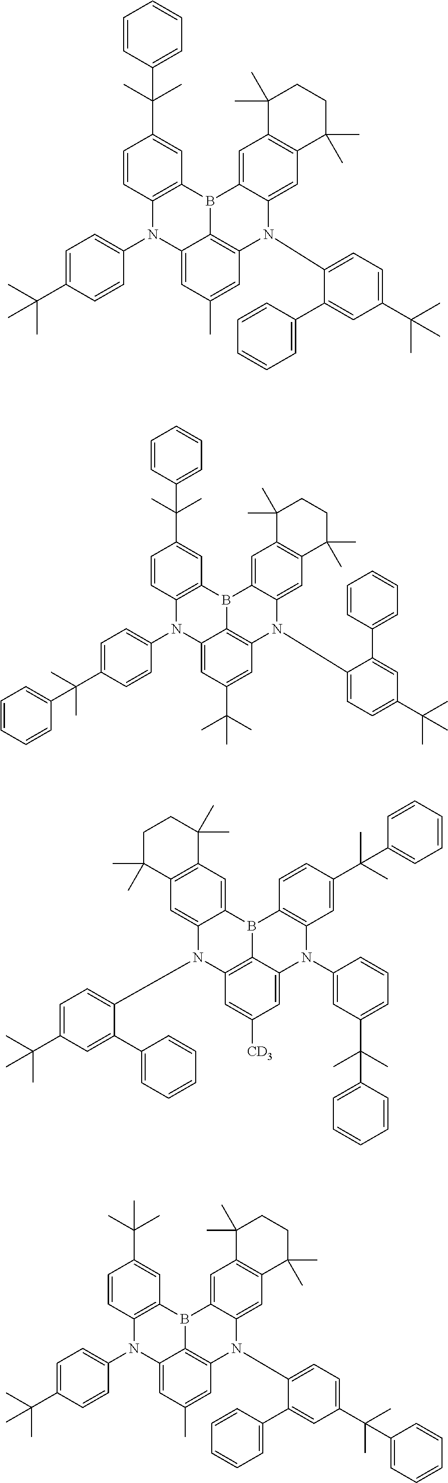

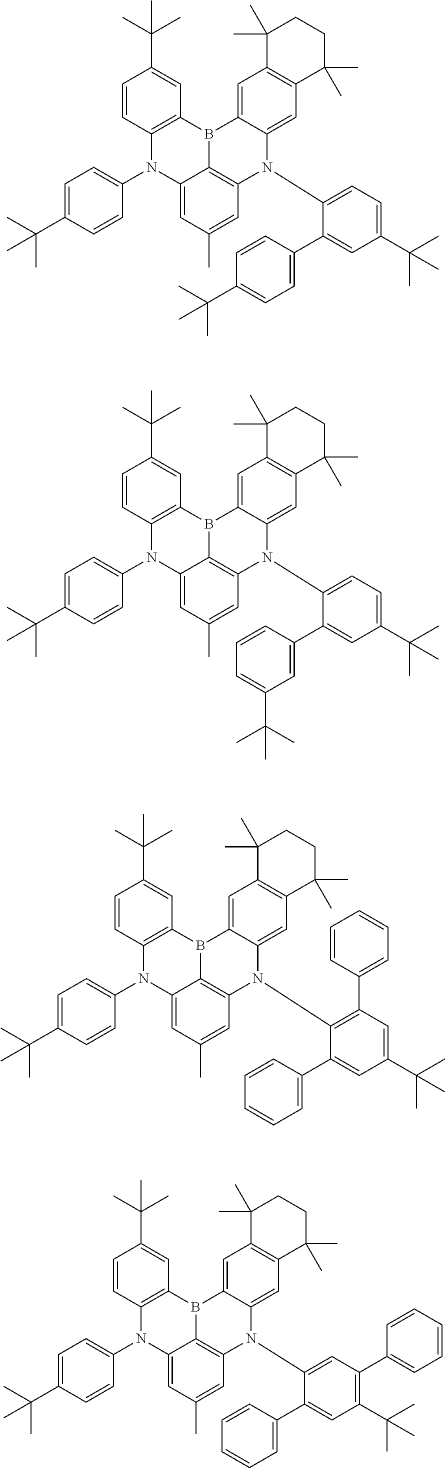

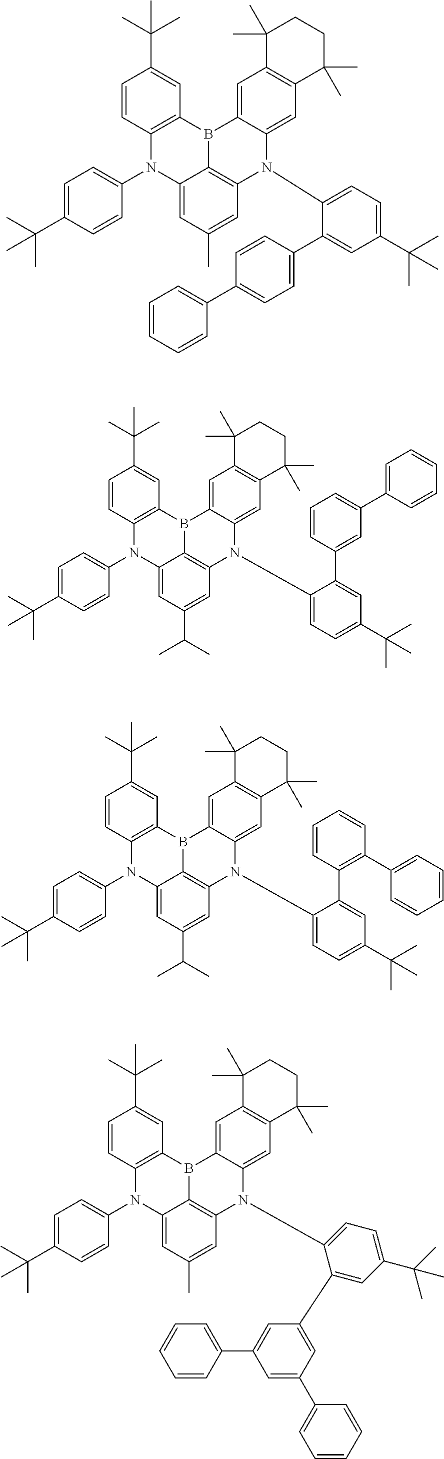

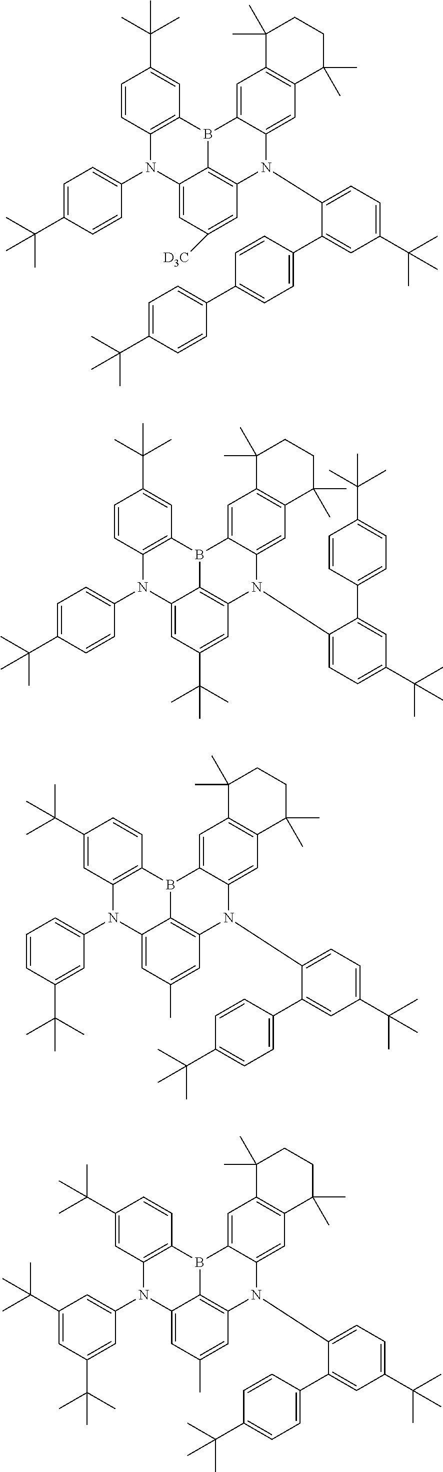

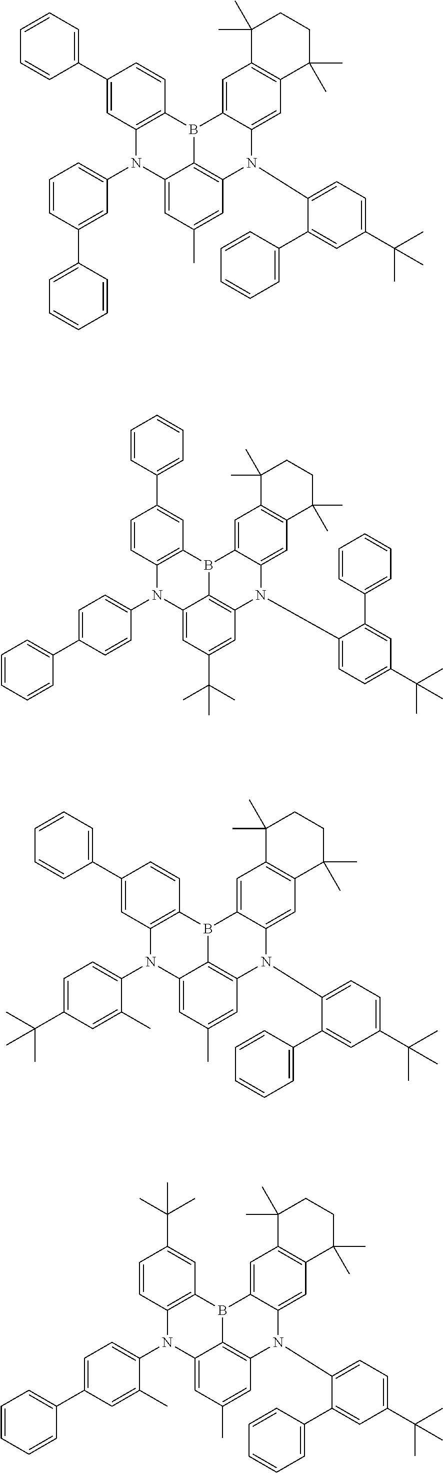

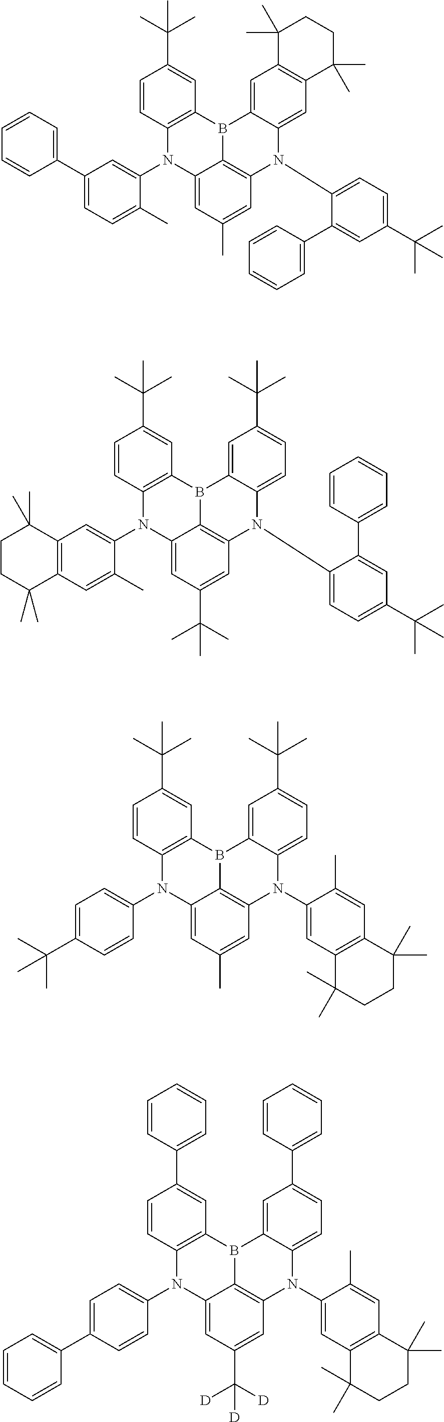

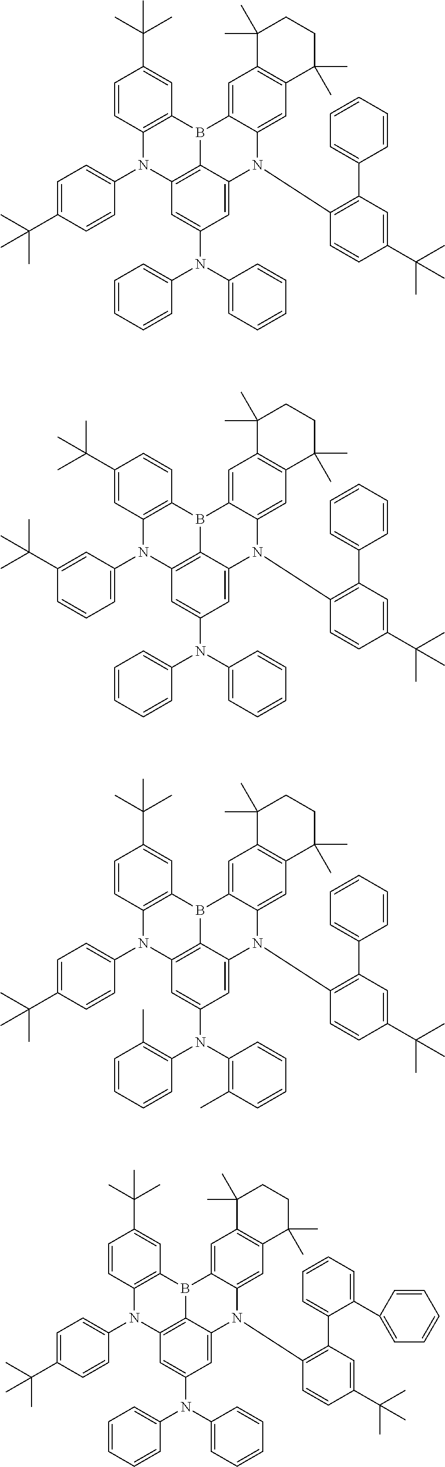

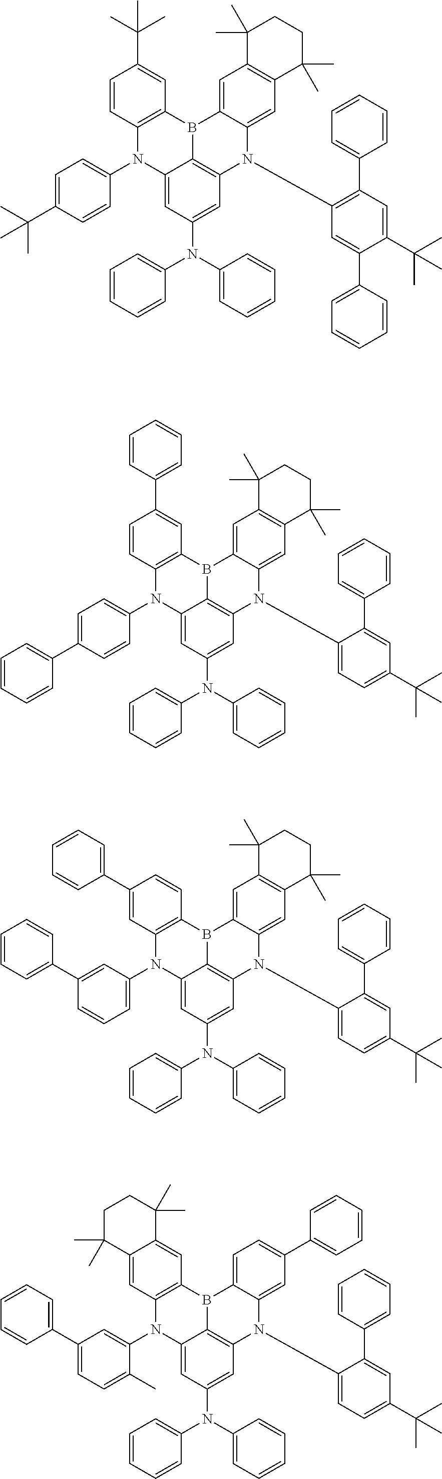

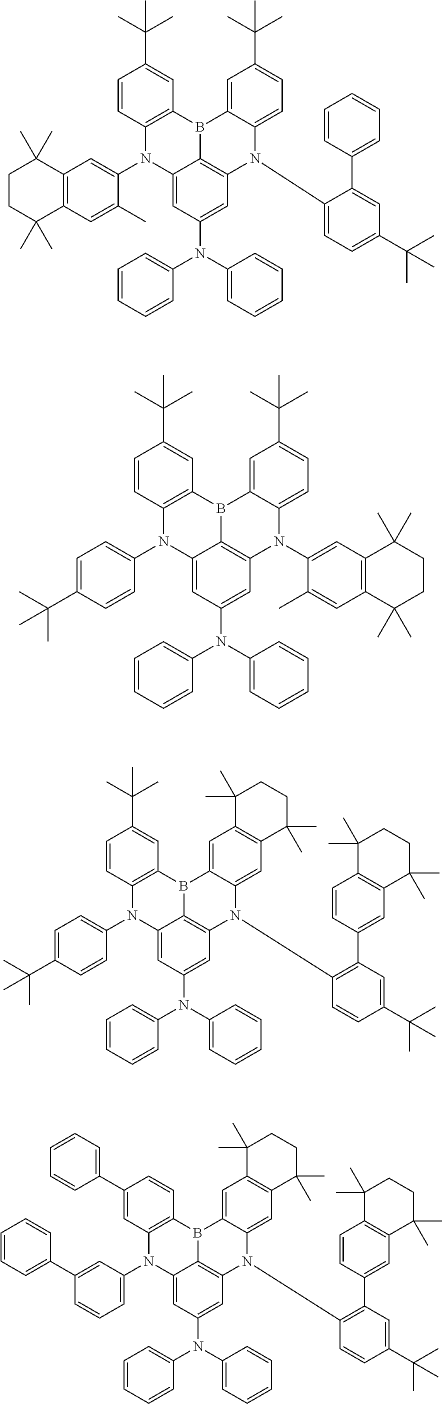

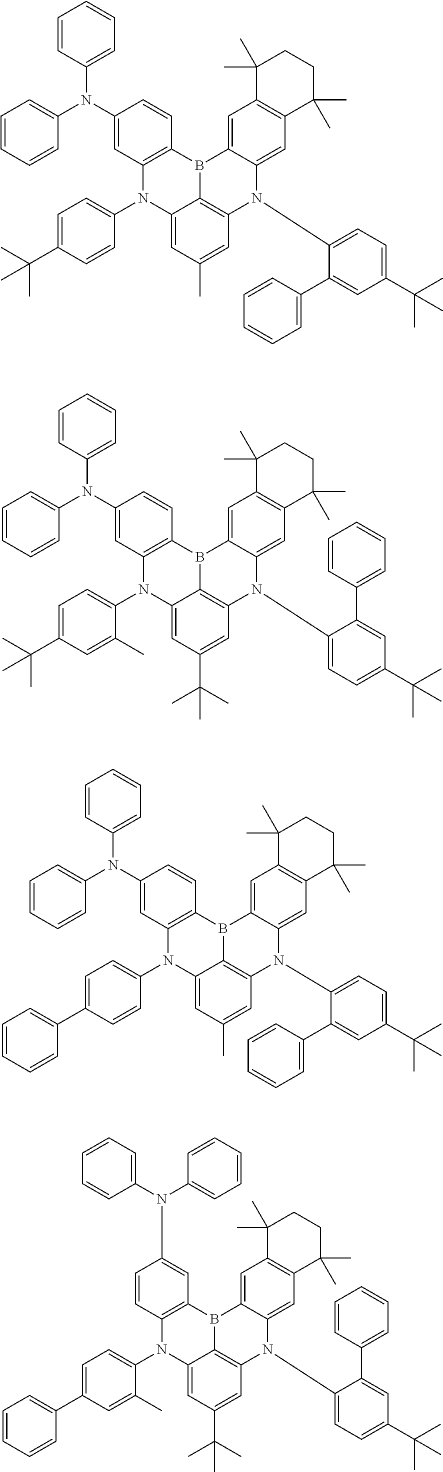

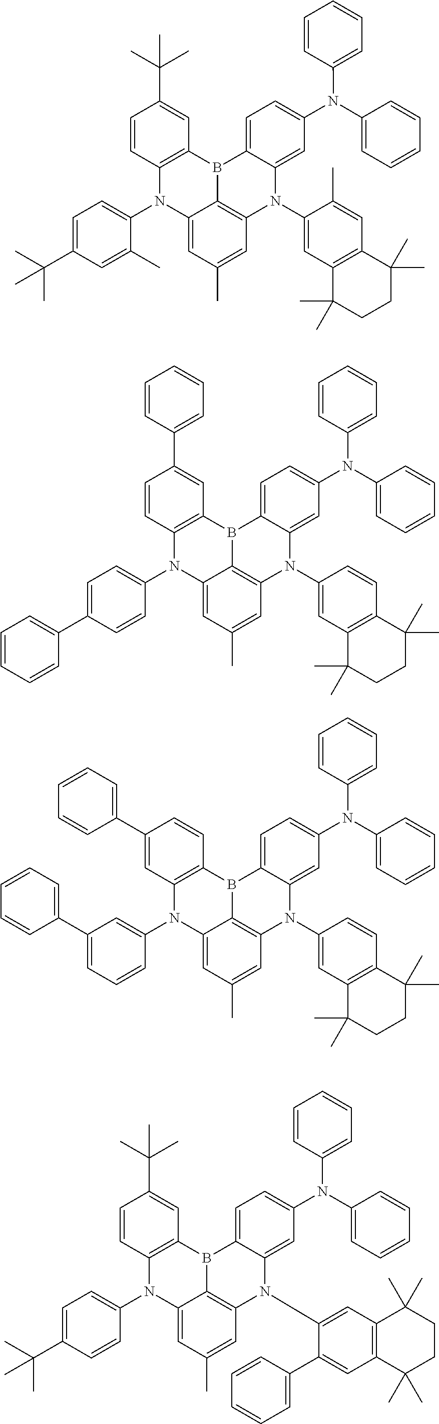

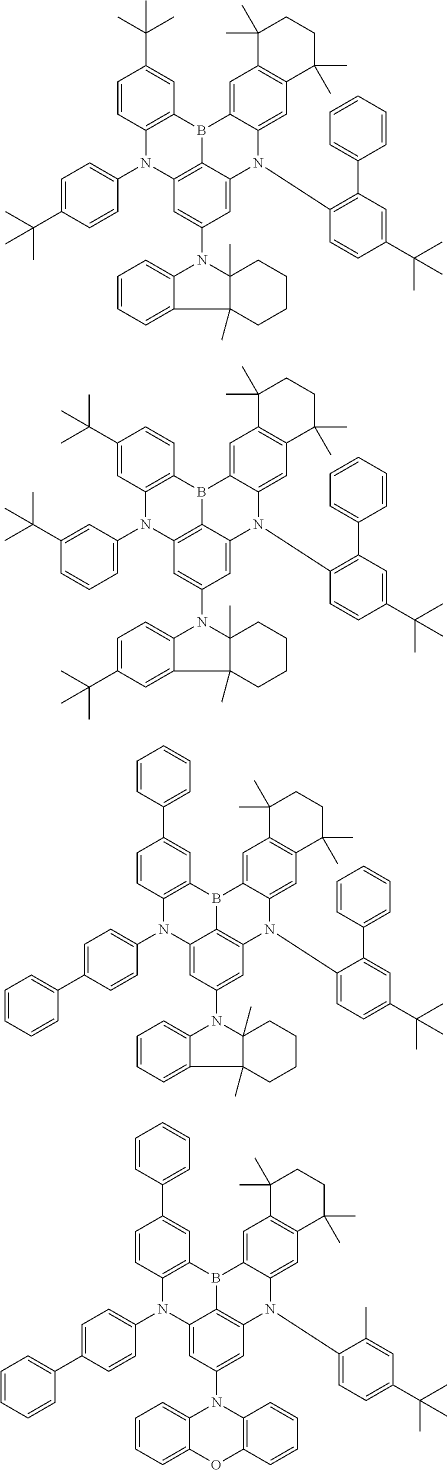

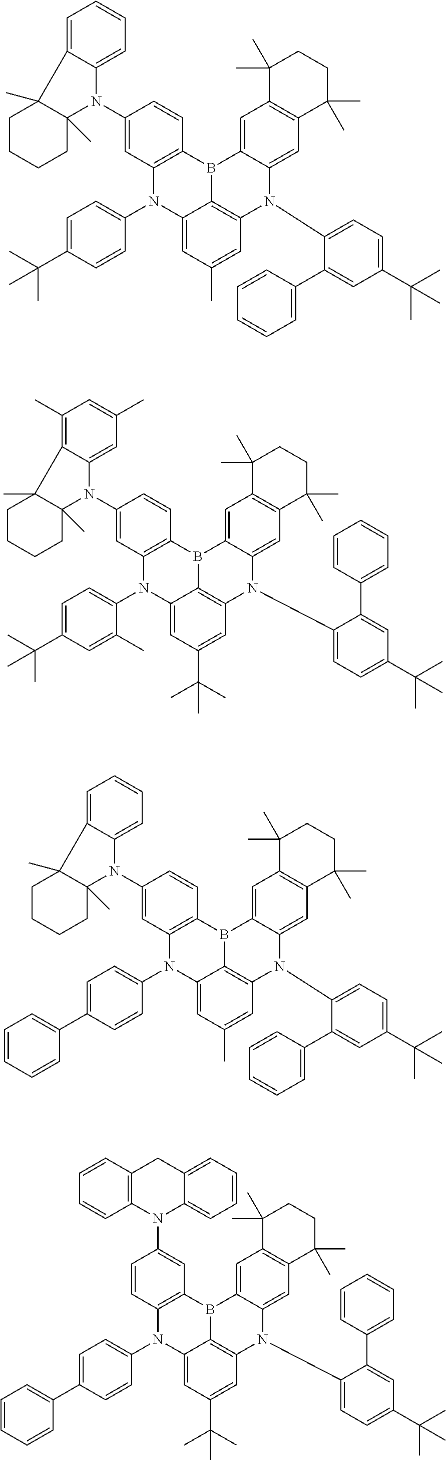

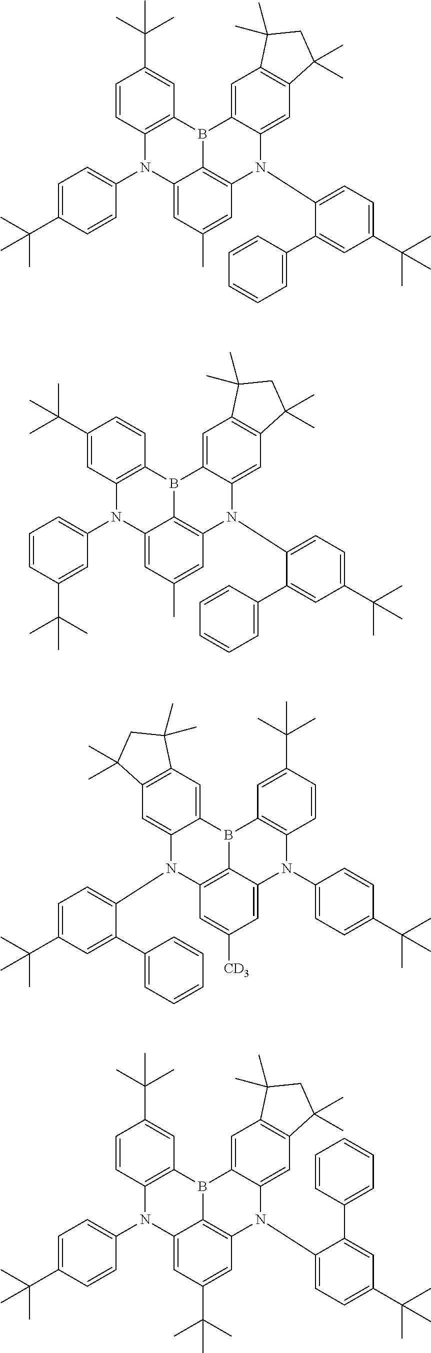

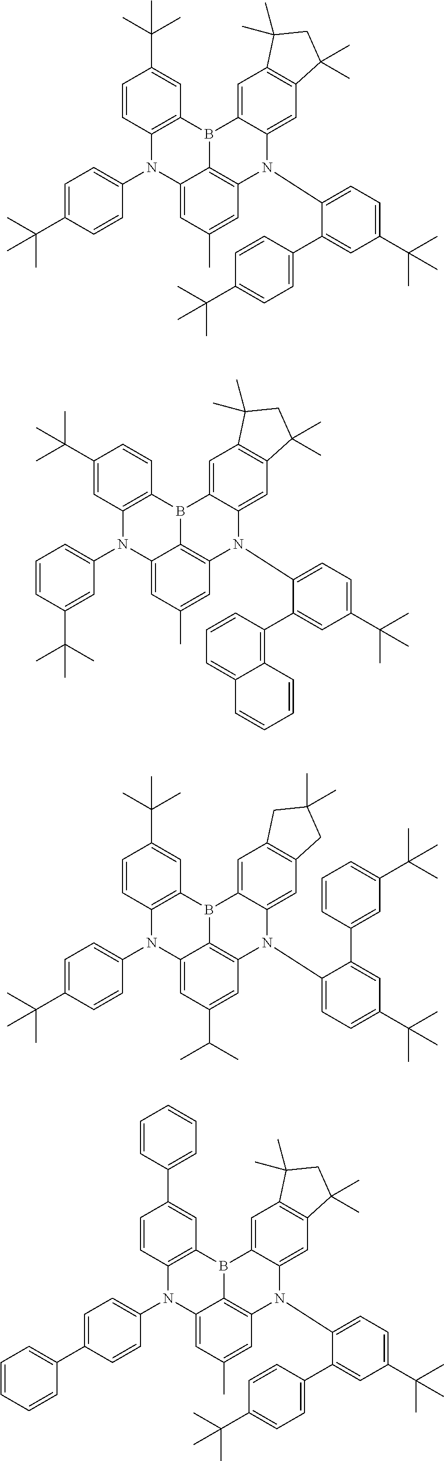

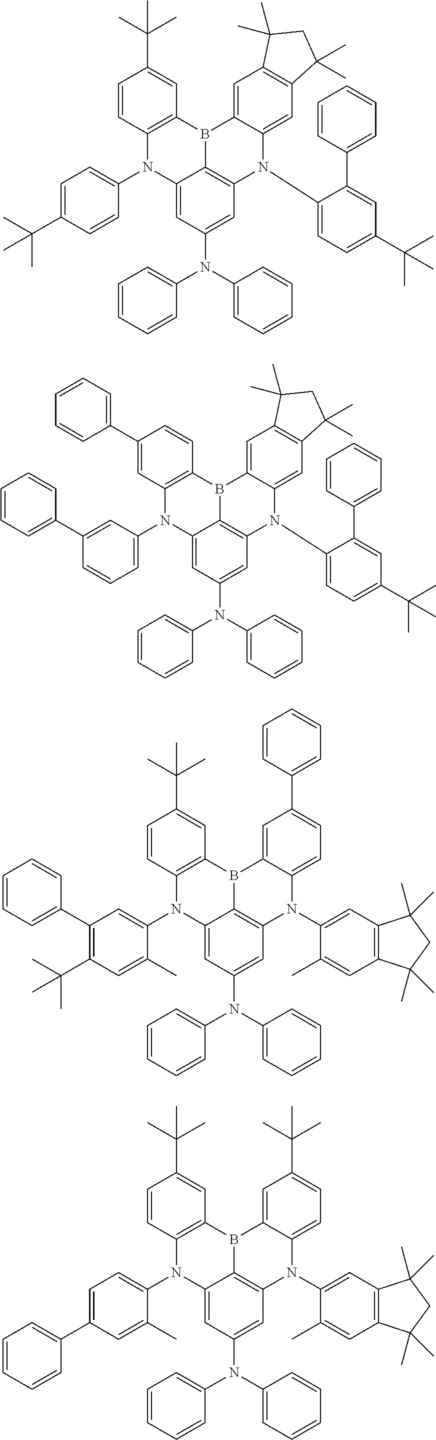

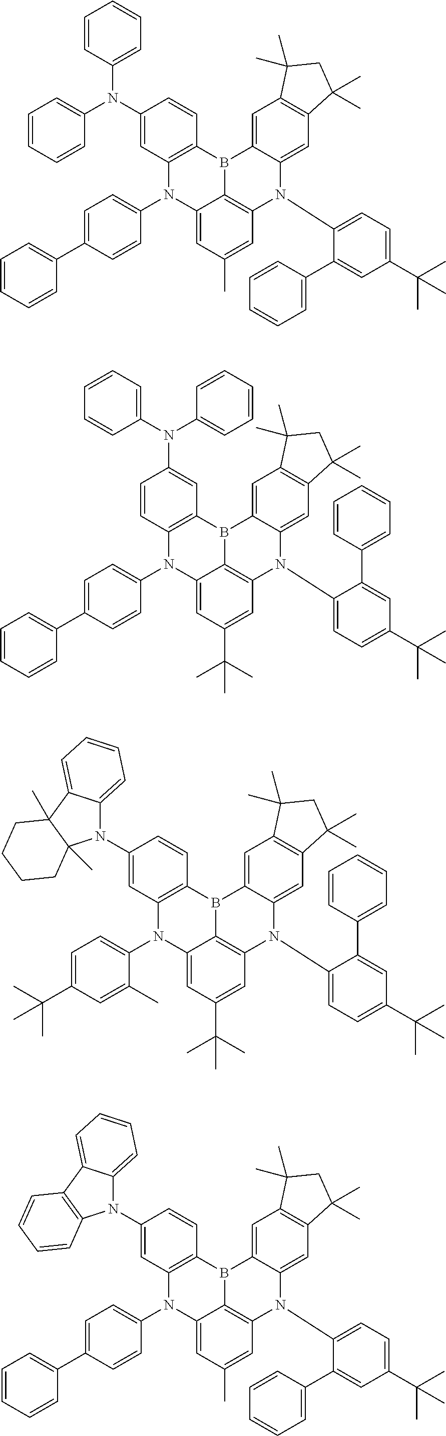

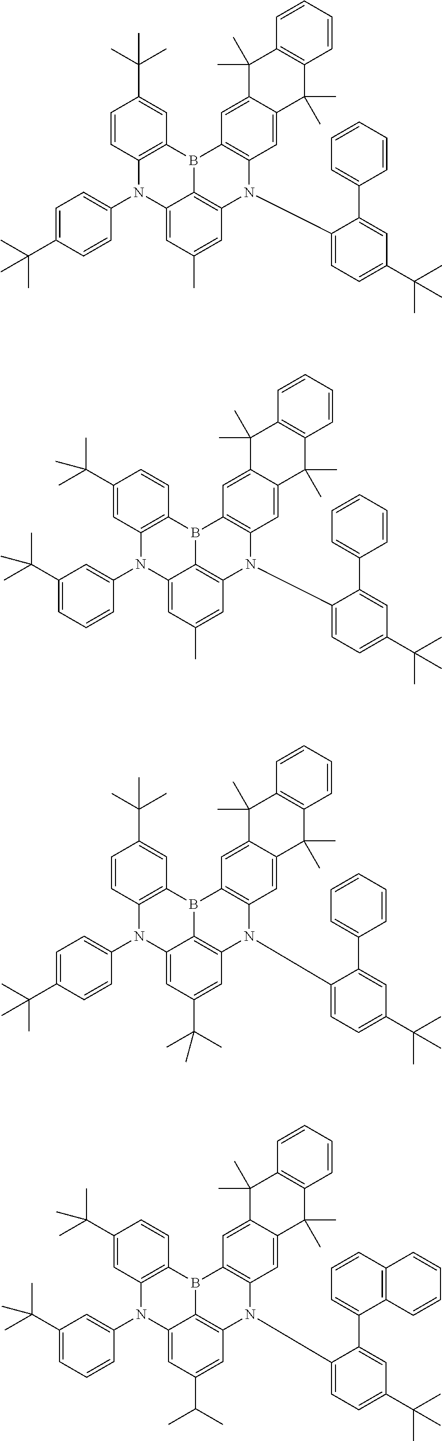

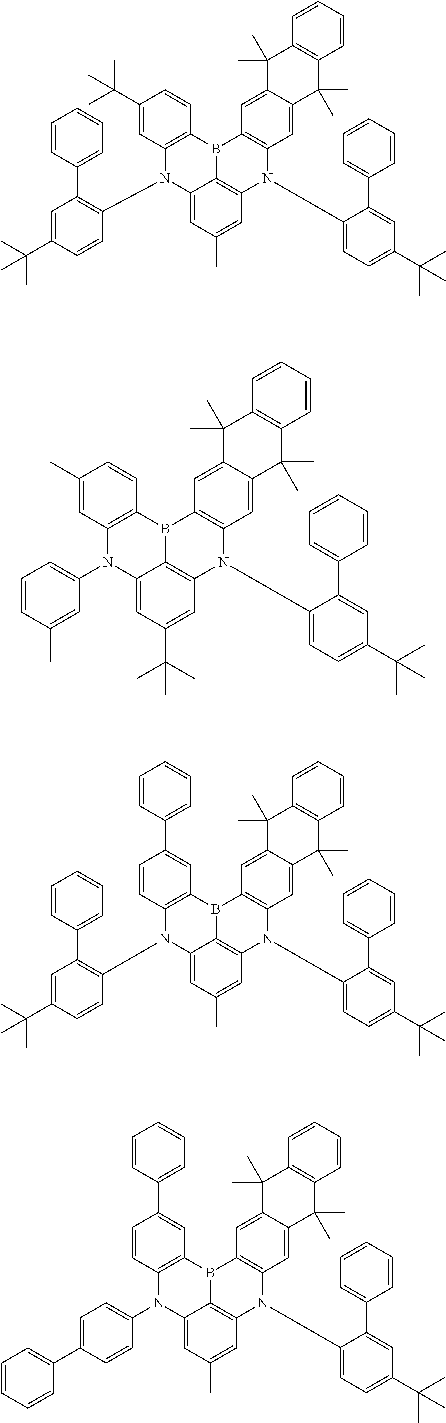

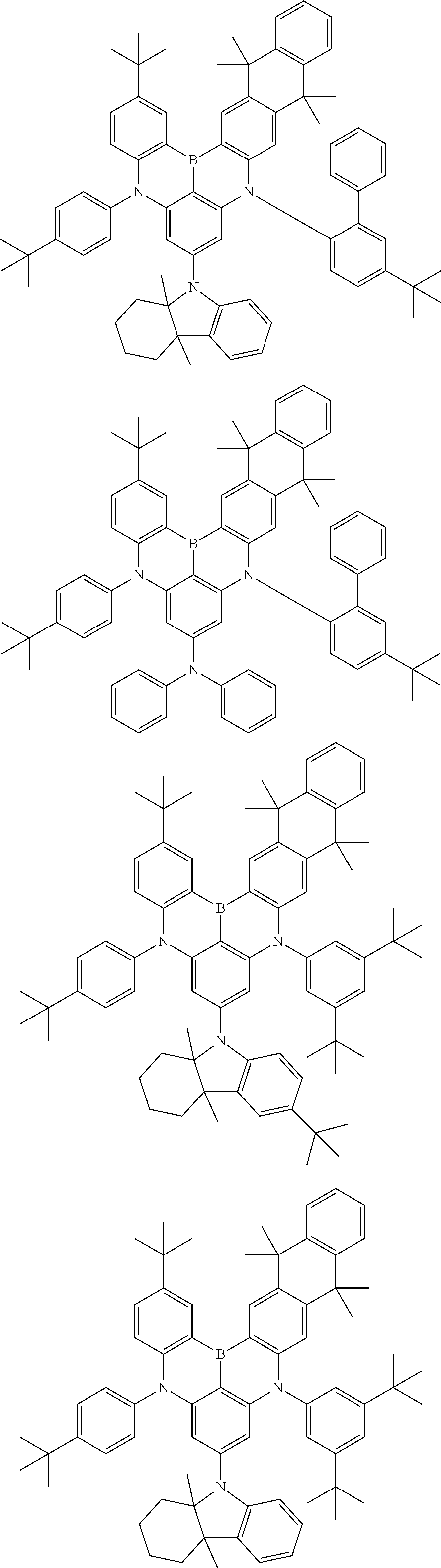

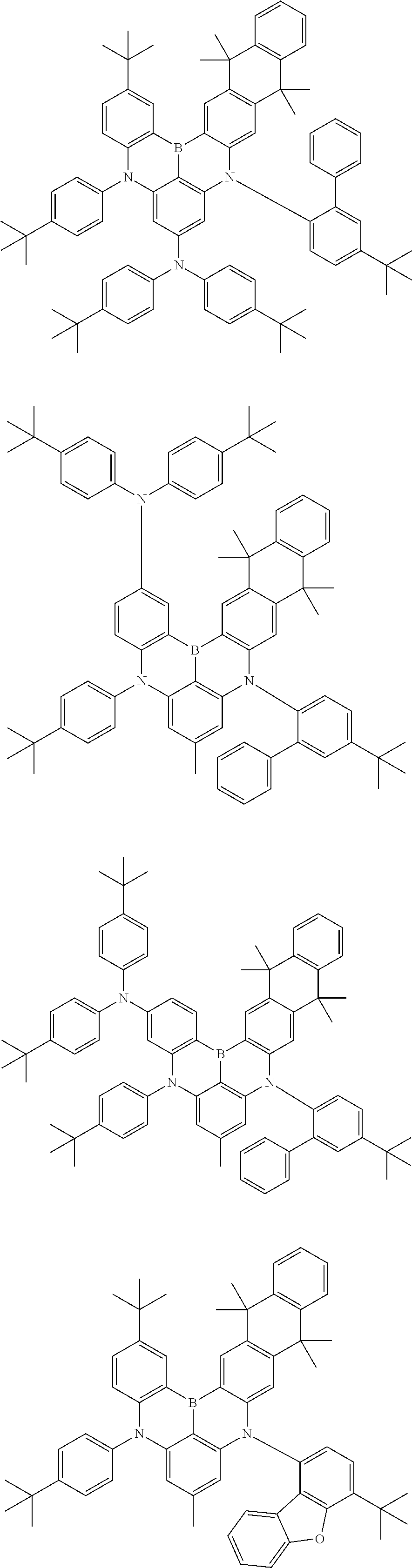

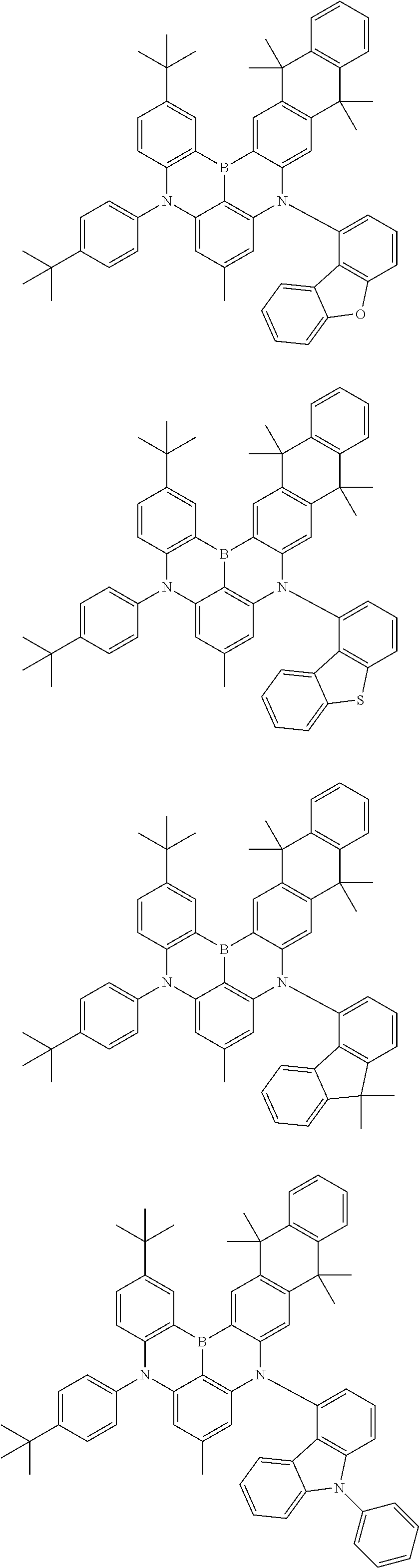 [SMILES: Cc1cc2c3c(c1)N(c1cccc4oc5ccccc5c14)c1cc4c(cc1B3c1cc(C(C)(C)C)ccc1N2c1ccc(C(C)(C)C)cc1)C(C)(C)c1ccccc1C4(C)C ; Cc1cc2c3c(c1)N(c1cccc4sc5ccccc5c14)c1cc4c(cc1B3c1cc(C(C)(C)C)ccc1N2c1ccc(C(C)(C)C)cc1)C(C)(C)c1ccccc1C4(C)C ; Cc1cc2c3c(c1)N(c1cccc4c1-c1ccccc1C4(C)C)c1cc4c(cc1B3c1cc(C(C)(C)C)ccc1N2c1ccc(C(C)(C)C)cc1)C(C)(C)c1ccccc1C4(C)C ; Cc1cc2c3c(c1)N(c1cccc4c1c1ccccc1n4-c1ccccc1)c1cc4c(cc1B3c1cc(C(C)(C)C)ccc1N2c1ccc(C(C)(C)C)cc1)C(C)(C)c1ccccc1C4(C)C]

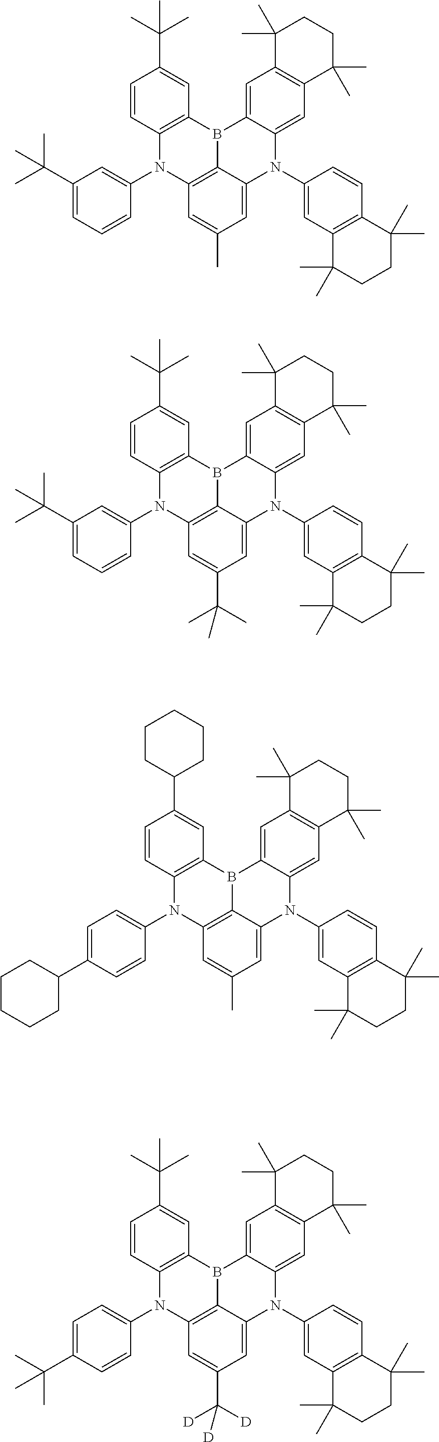

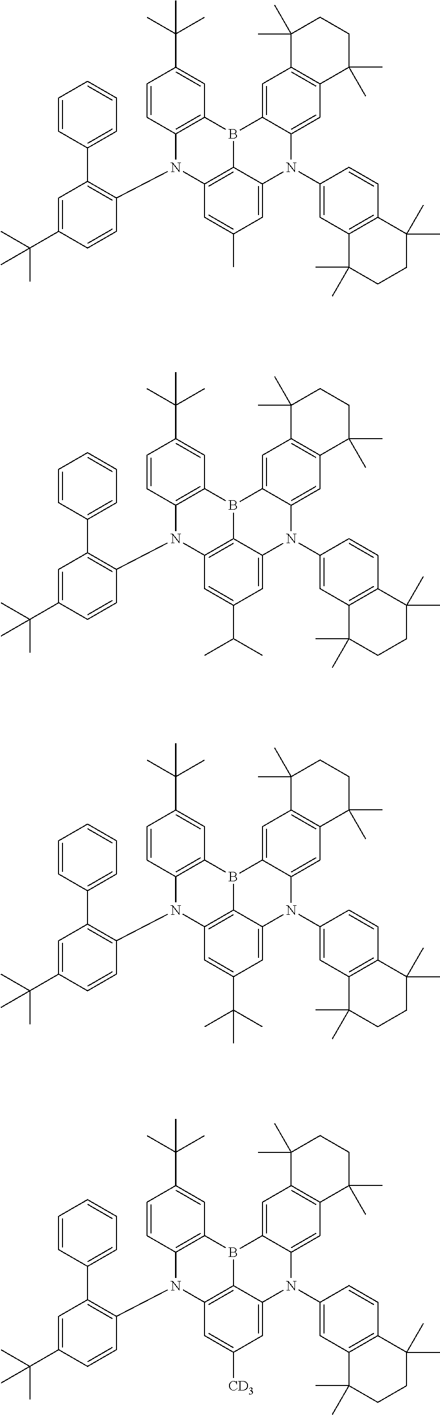

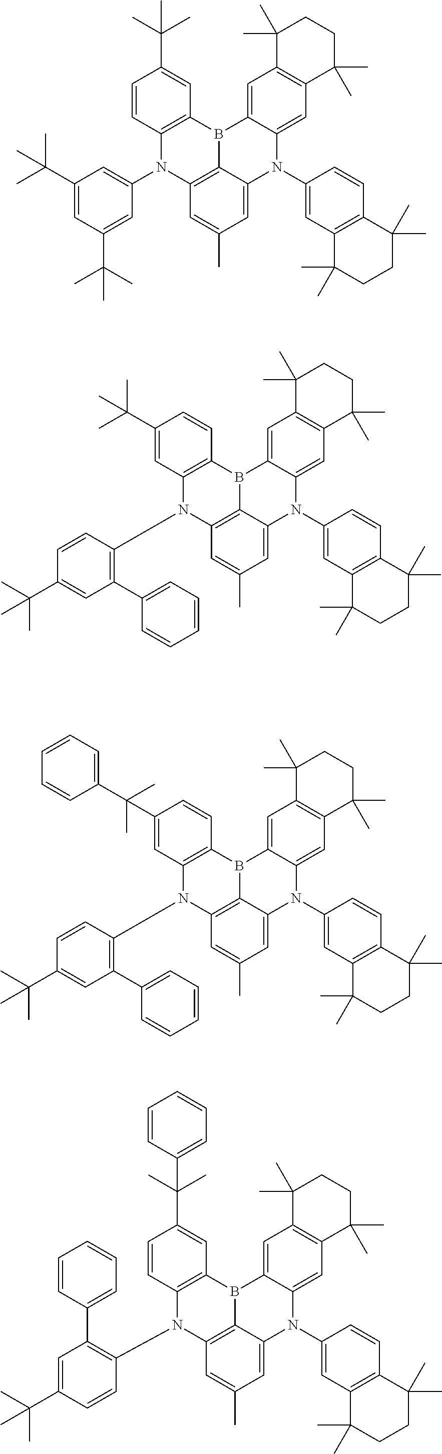

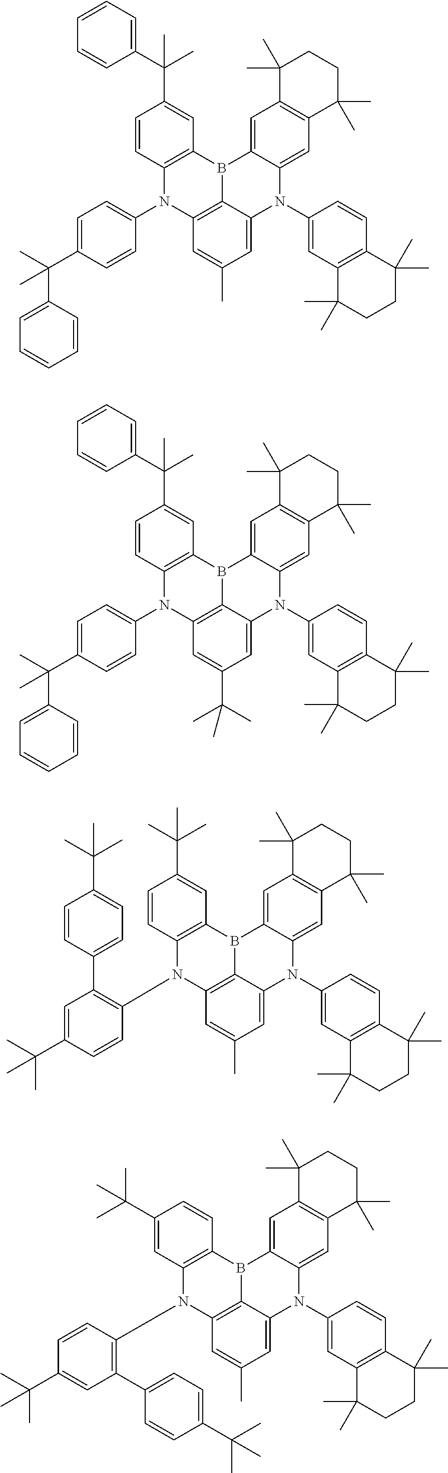

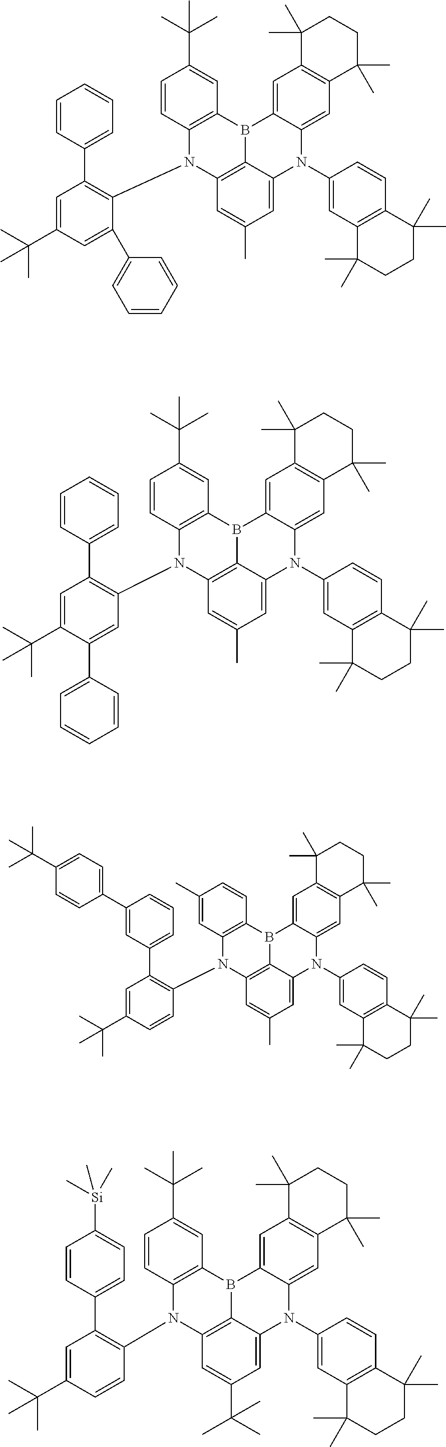

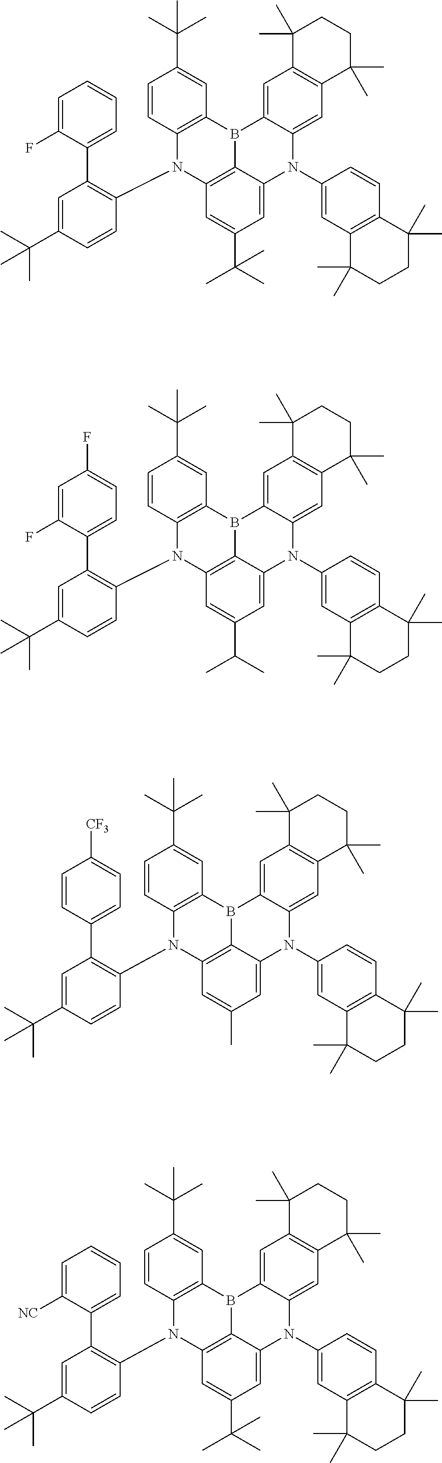

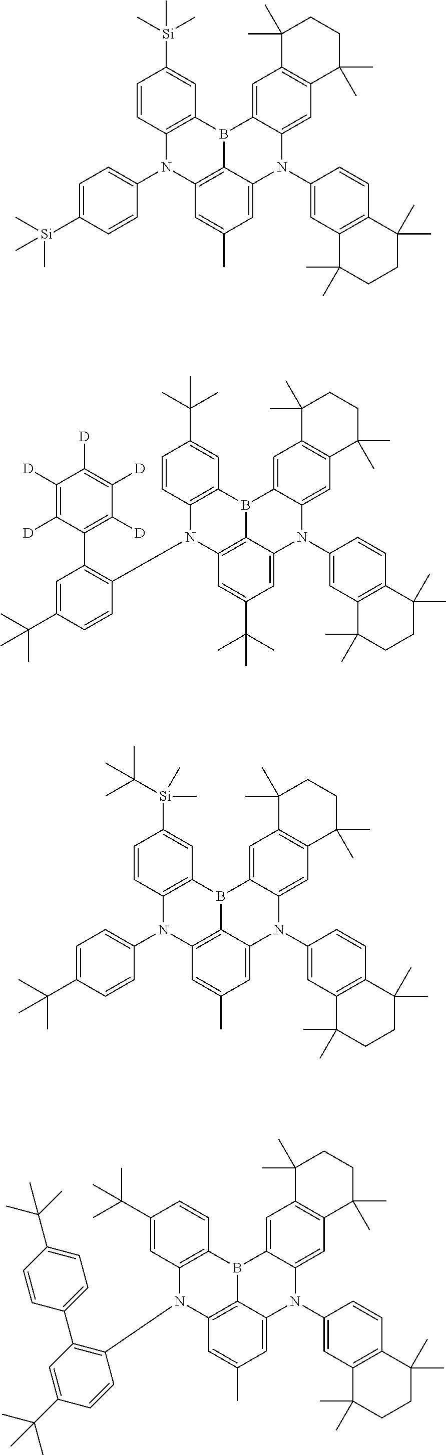

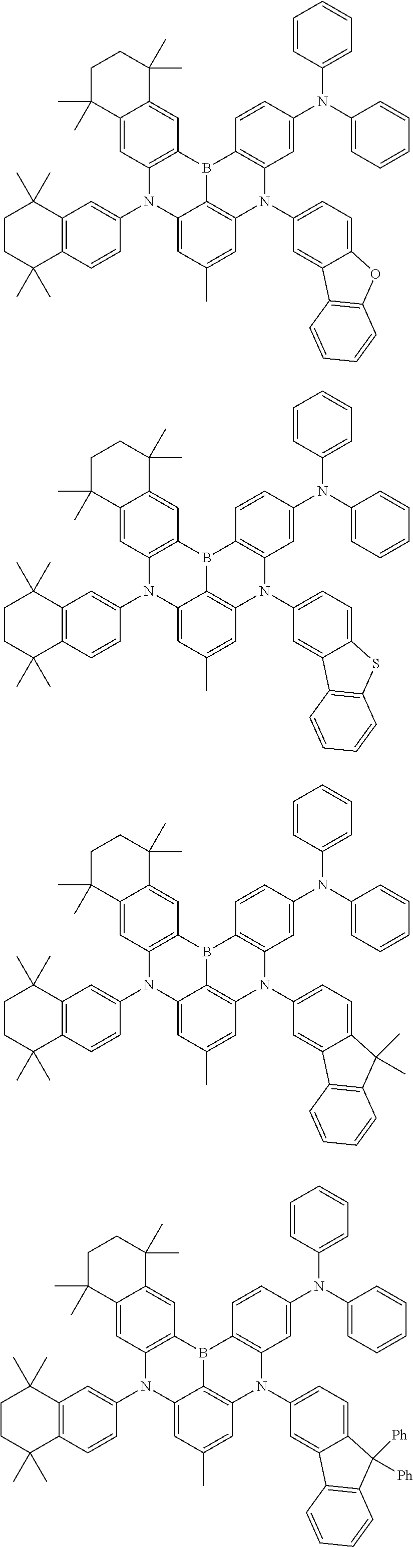

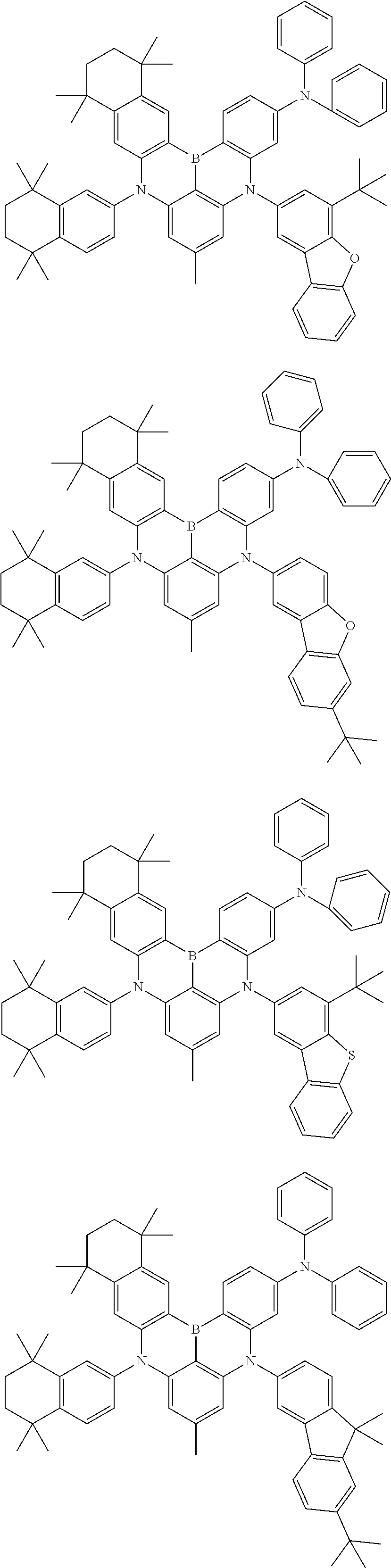

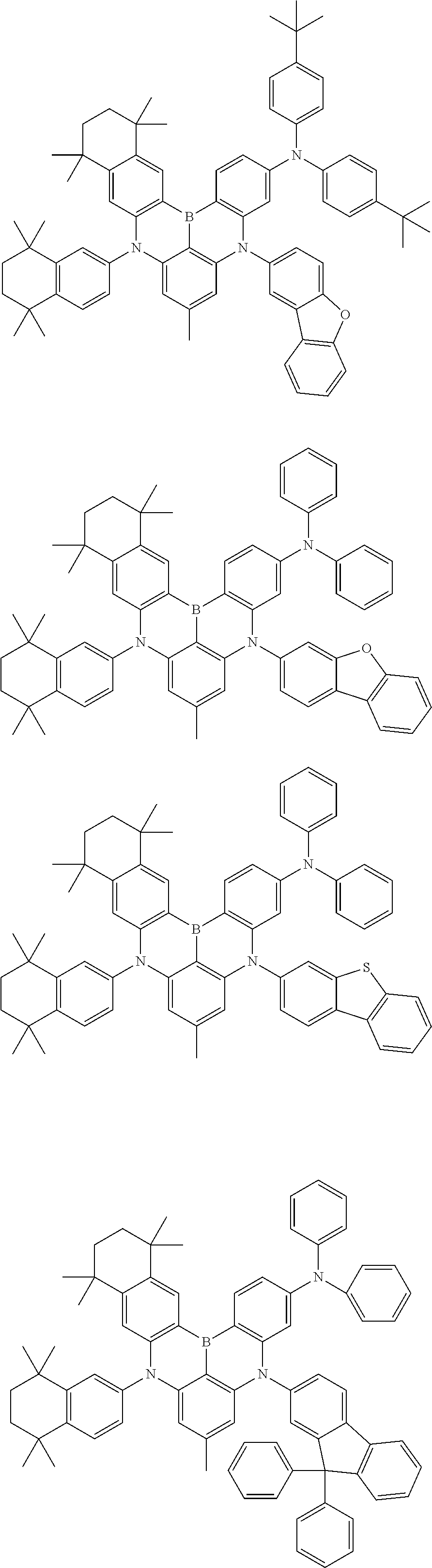

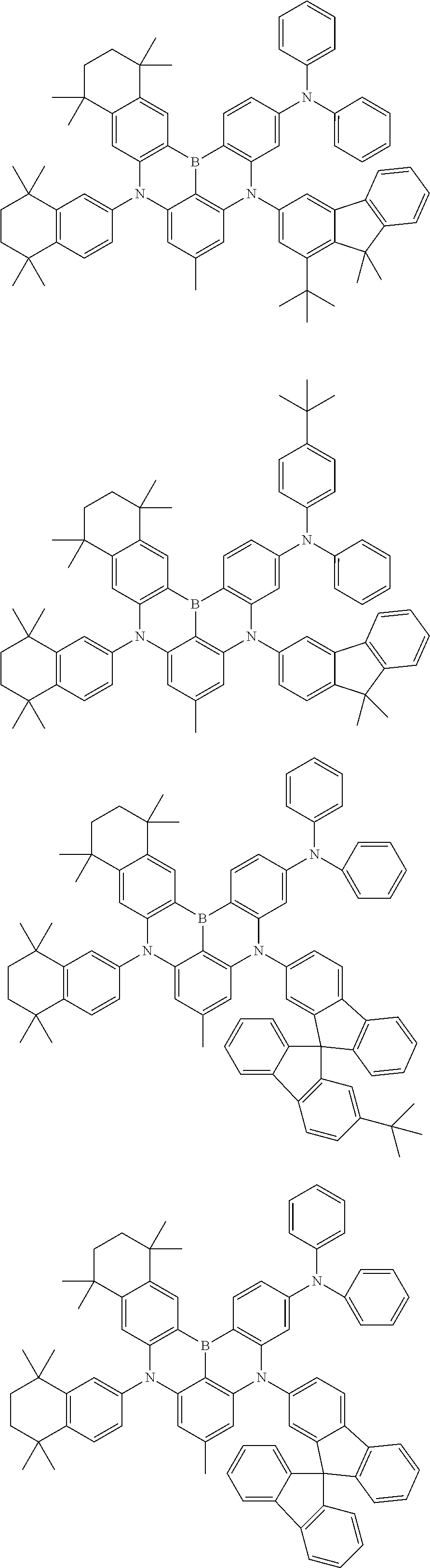

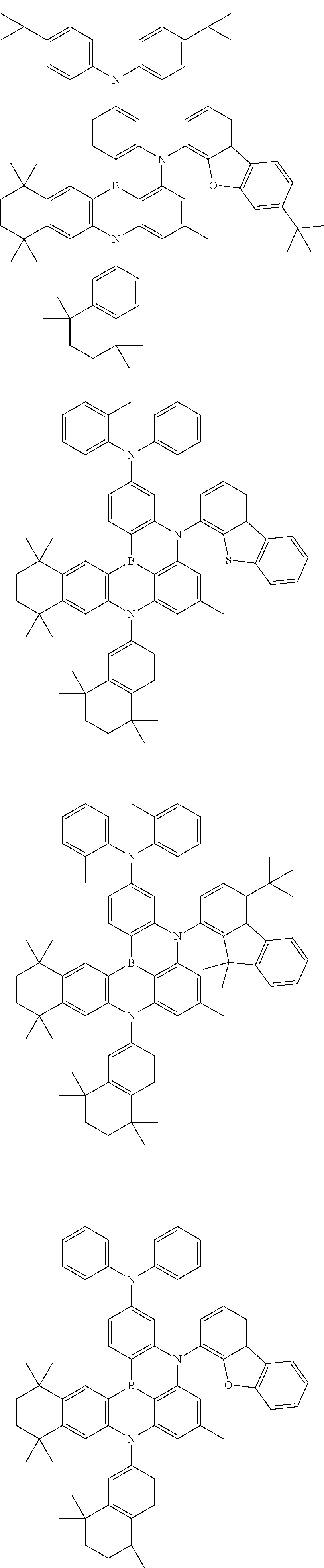

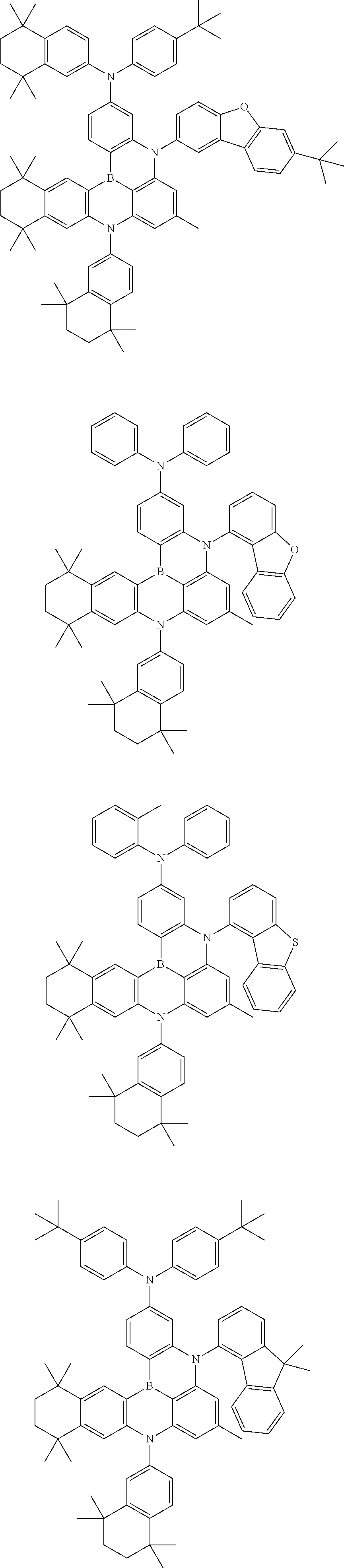

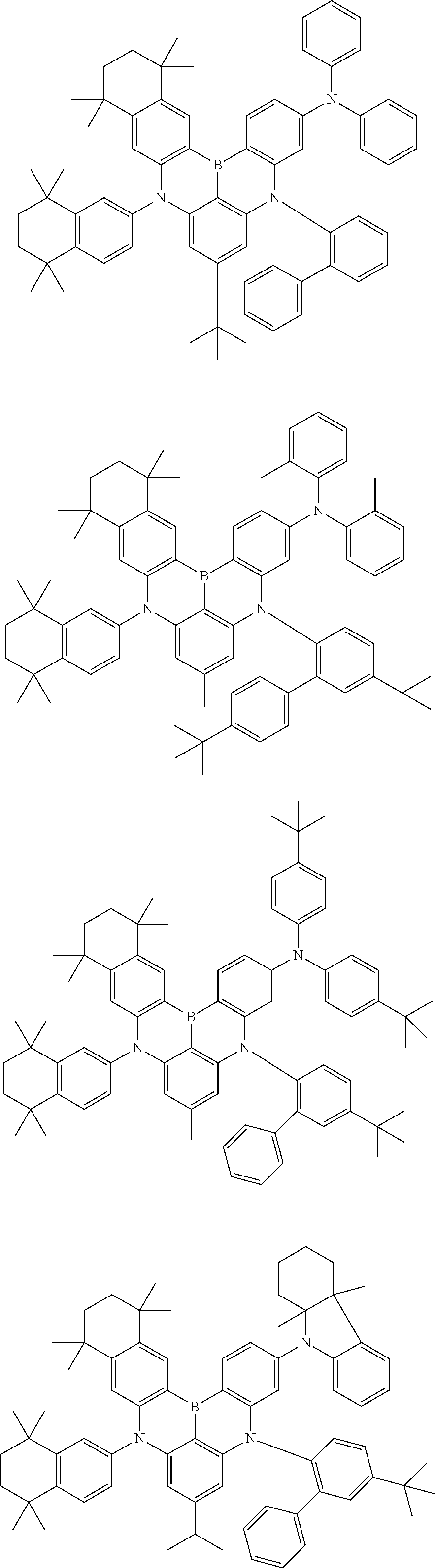

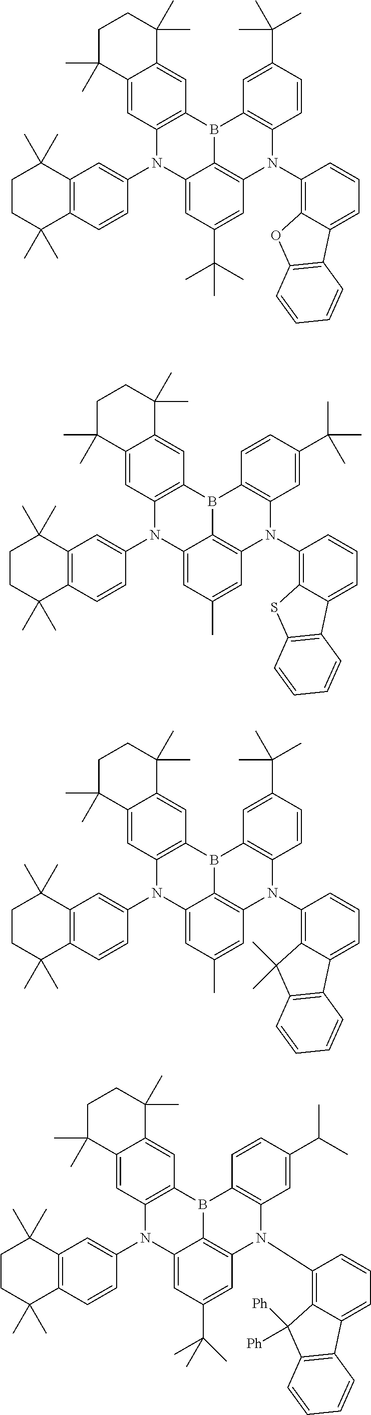 [SMILES: CC(C)(C)c1ccc2c(c1)B1c3cc4c(cc3N(c3ccc5c(c3)C(C)(C)CCC5(C)C)c3cc(C(C)(C)C)cc(c31)N2c1cccc2c1oc1ccccc12)C(C)(C)CCC4(C)C ; Cc1cc2c3c(c1)N(c1cccc4c1sc1ccccc14)c1cc(C(C)(C)C)ccc1B3c1cc3c(cc1N2c1ccc2c(c1)C(C)(C)CCC2(C)C)C(C)(C)CCC3(C)C ; Cc1cc2c3c(c1)N(c1cccc4c1C(C)(C)c1ccccc1-4)c1ccc(C(C)(C)C)cc1B3c1cc3c(cc1N2c1ccc2c(c1)C(C)(C)CCC2(C)C)C(C)(C)CCC3(C)C ; CC(C)c1ccc2c(c1)N(c1cccc3c1C(c1ccccc1)(c1ccccc1)c1ccccc1-3)c1cc(C(C)(C)C)cc3c1B2c1cc2c(cc1N3c1ccc3c(c1)C(C)(C)CCC3(C)C)C(C)(C)CCC2(C)C]

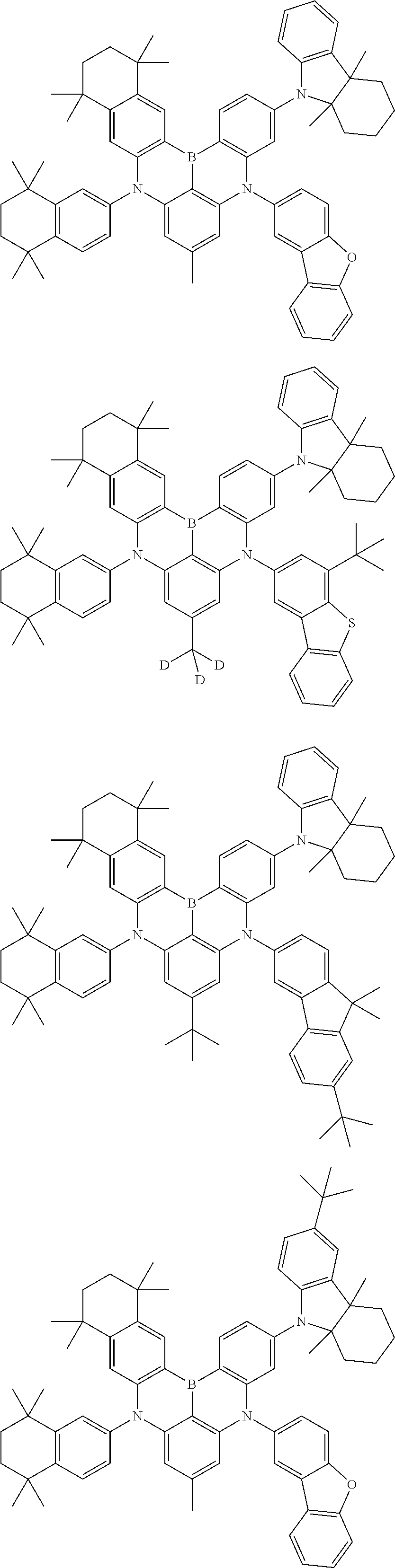

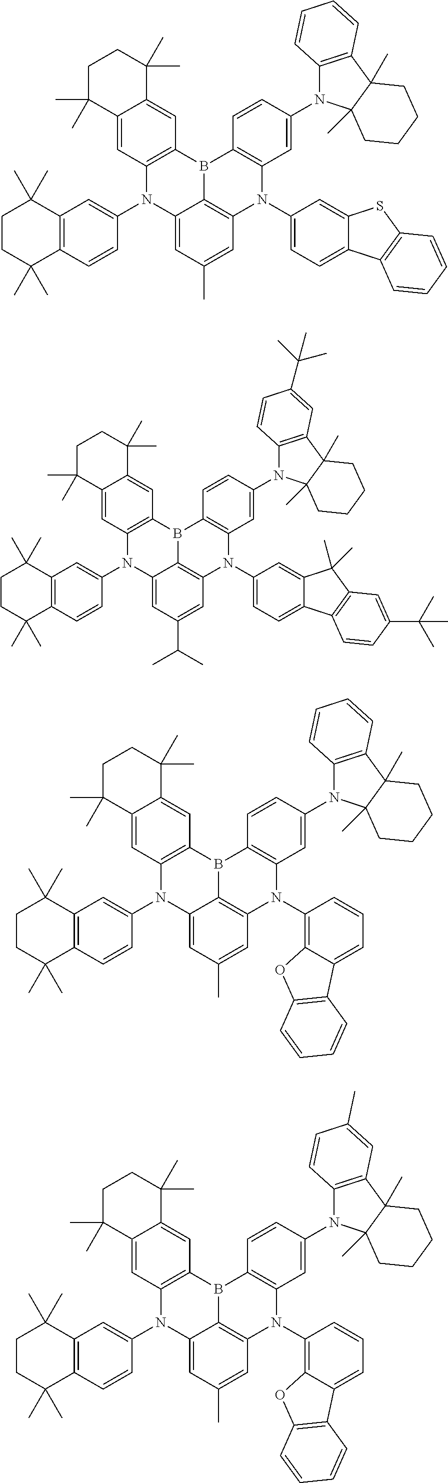

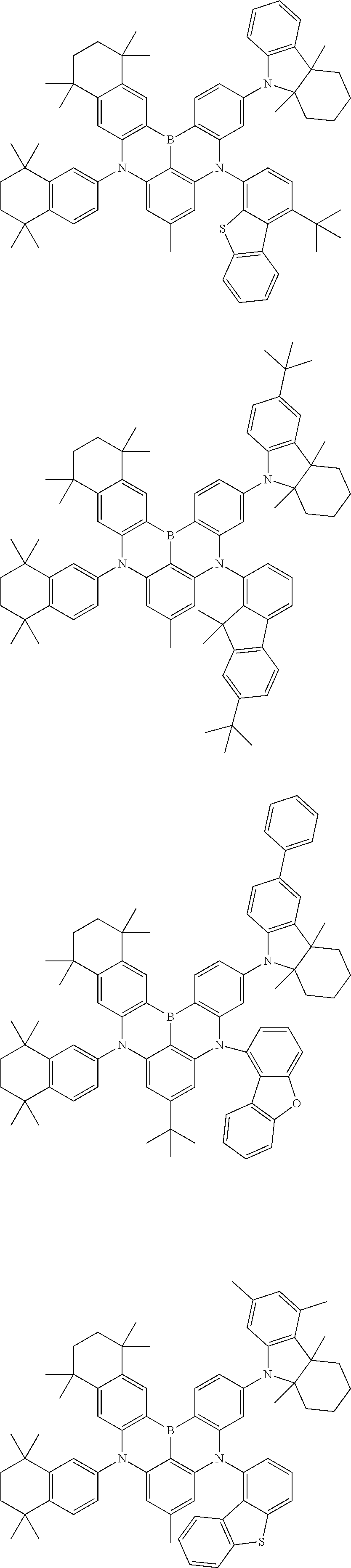

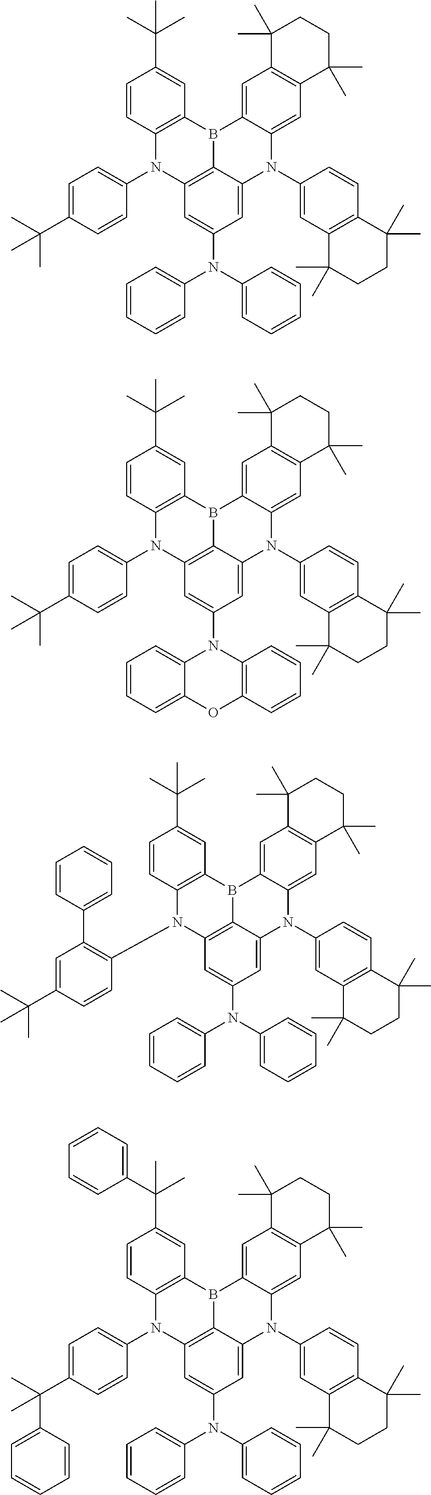

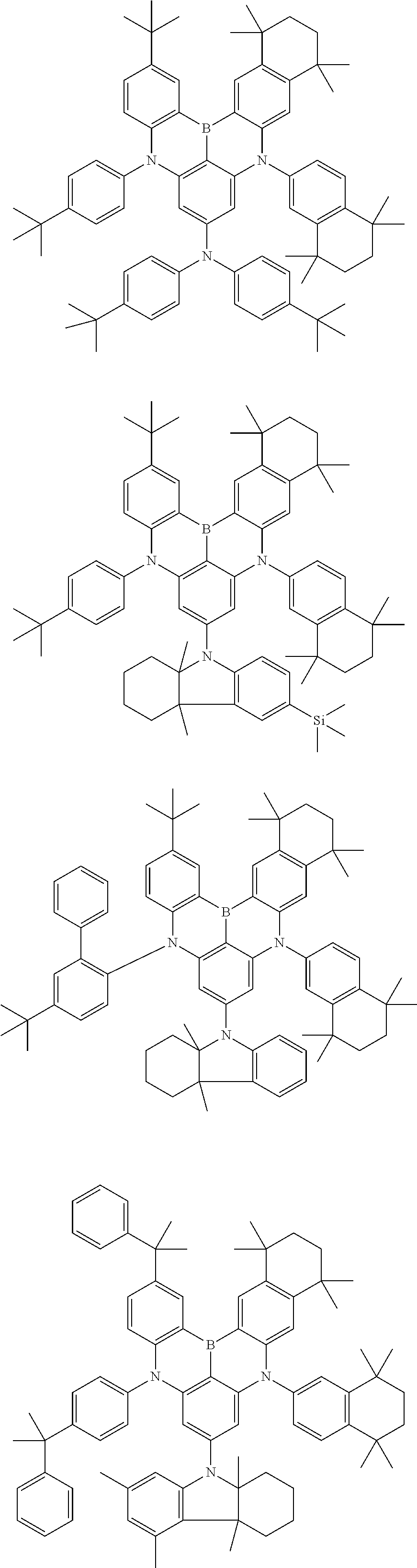

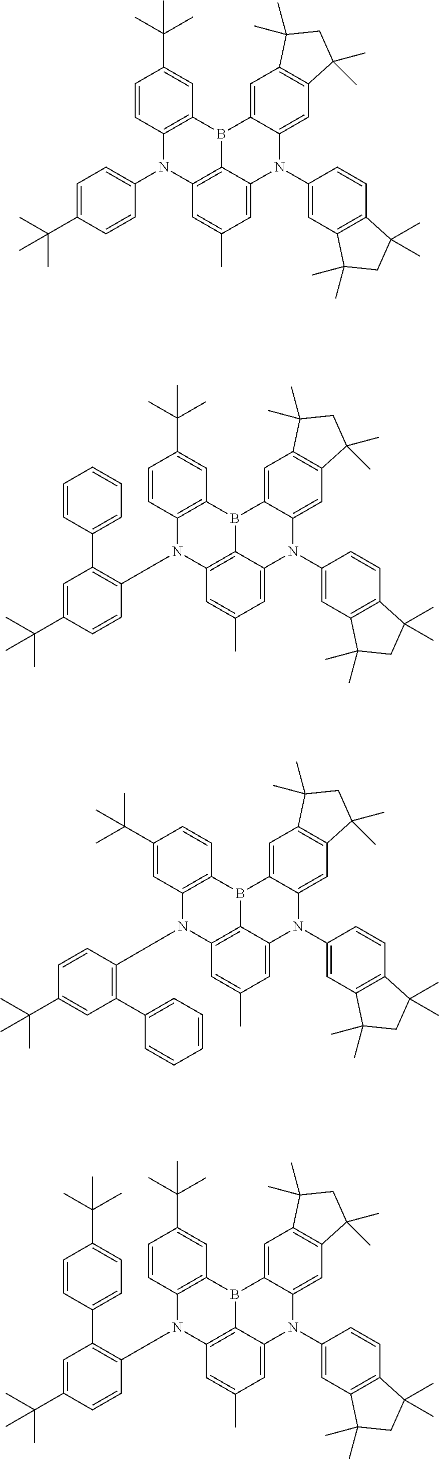

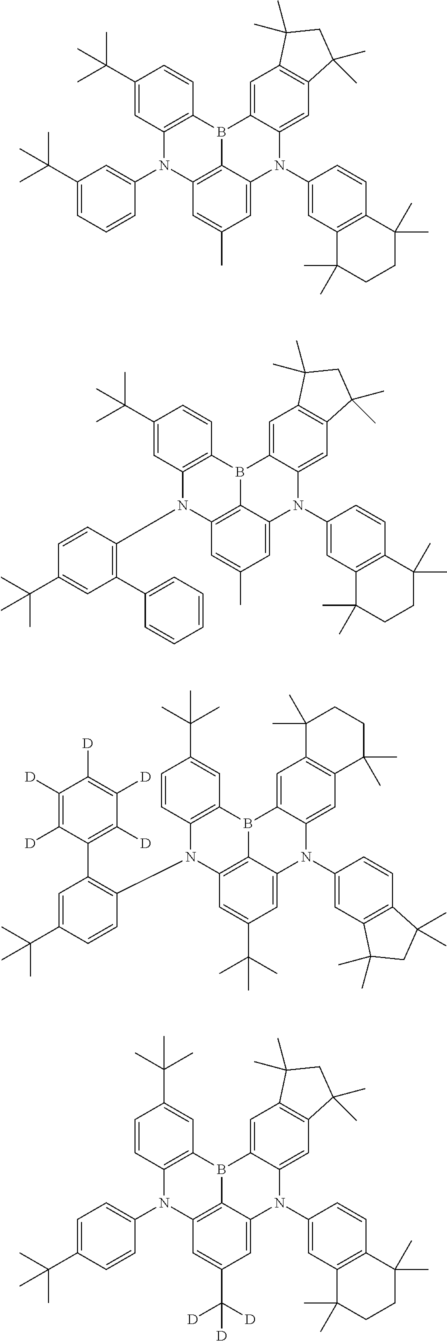

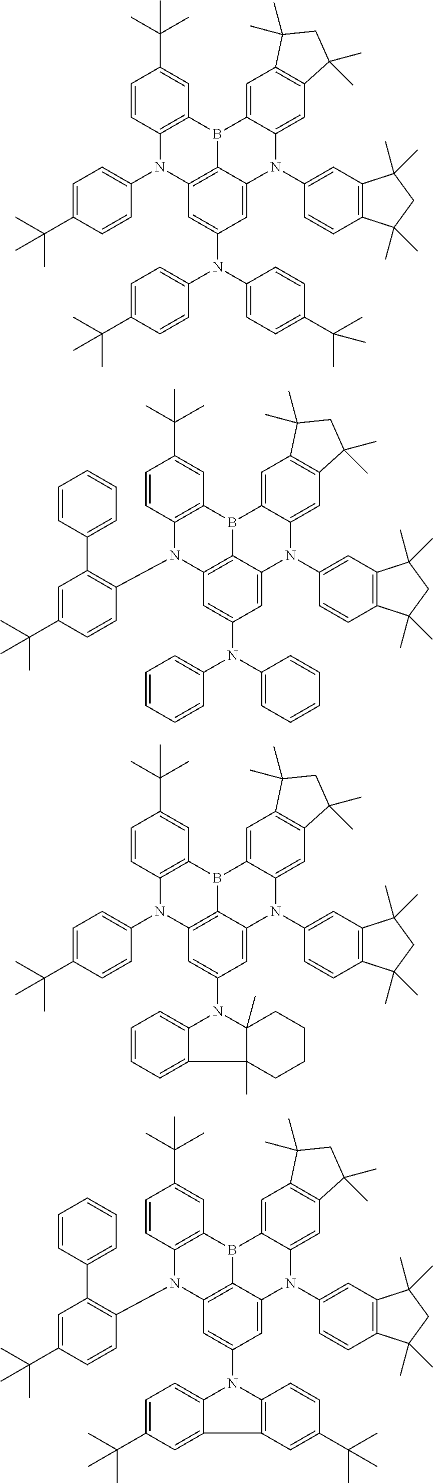

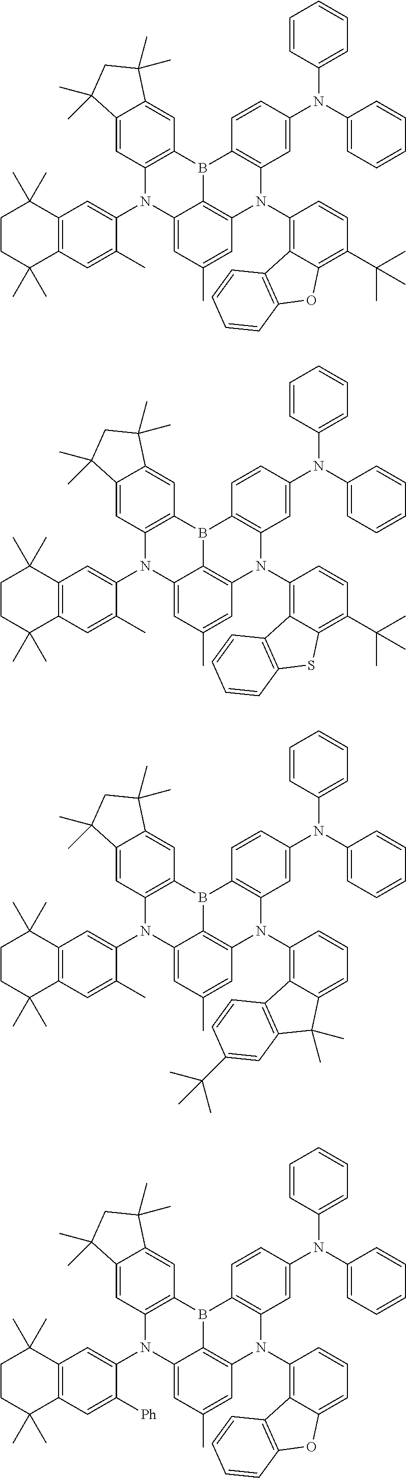

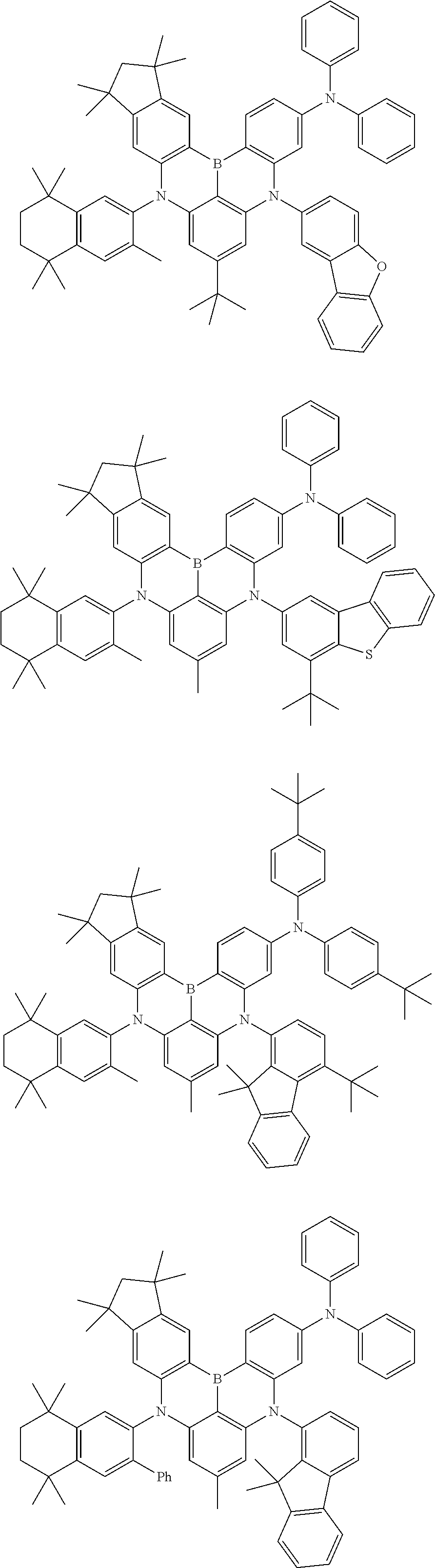

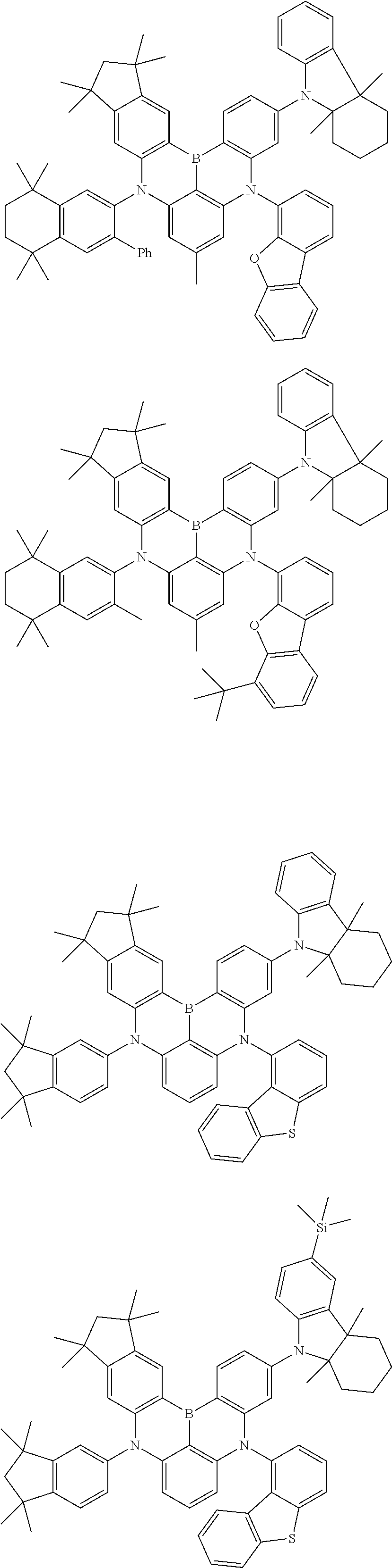

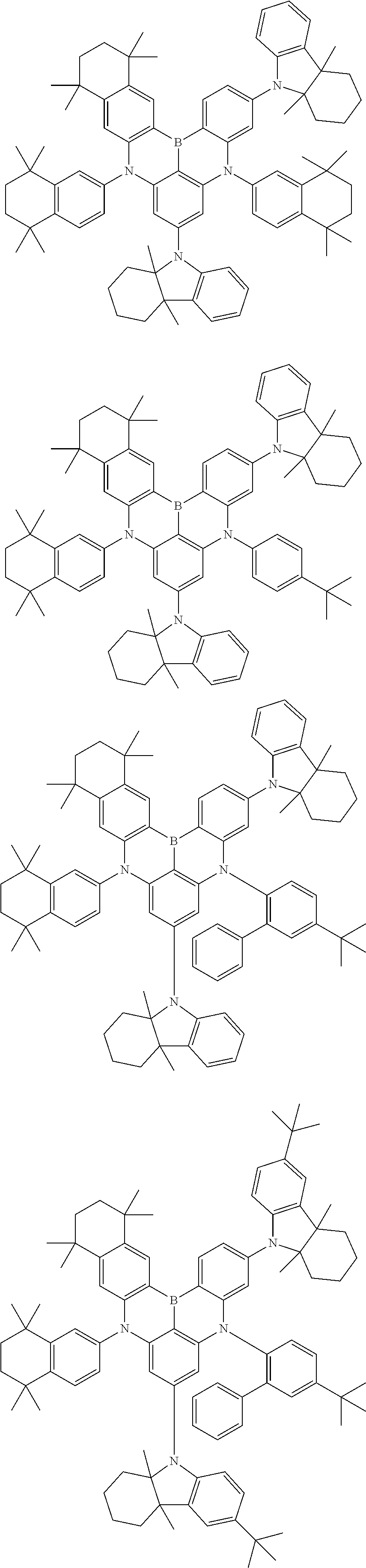

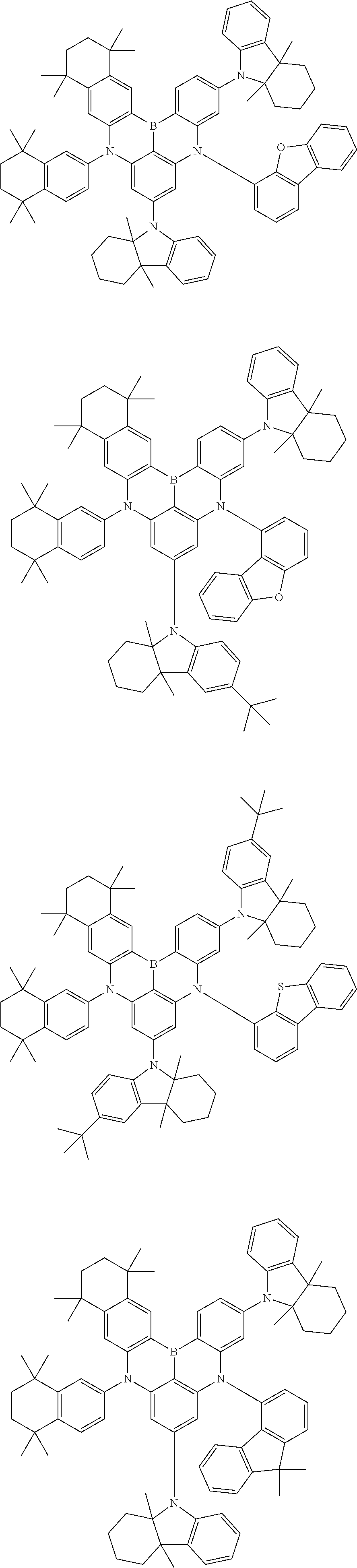

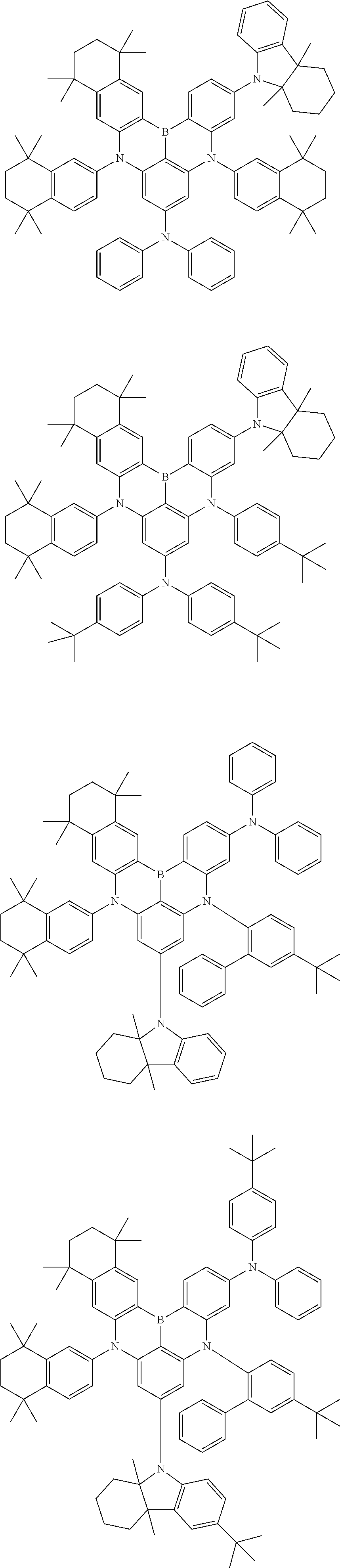

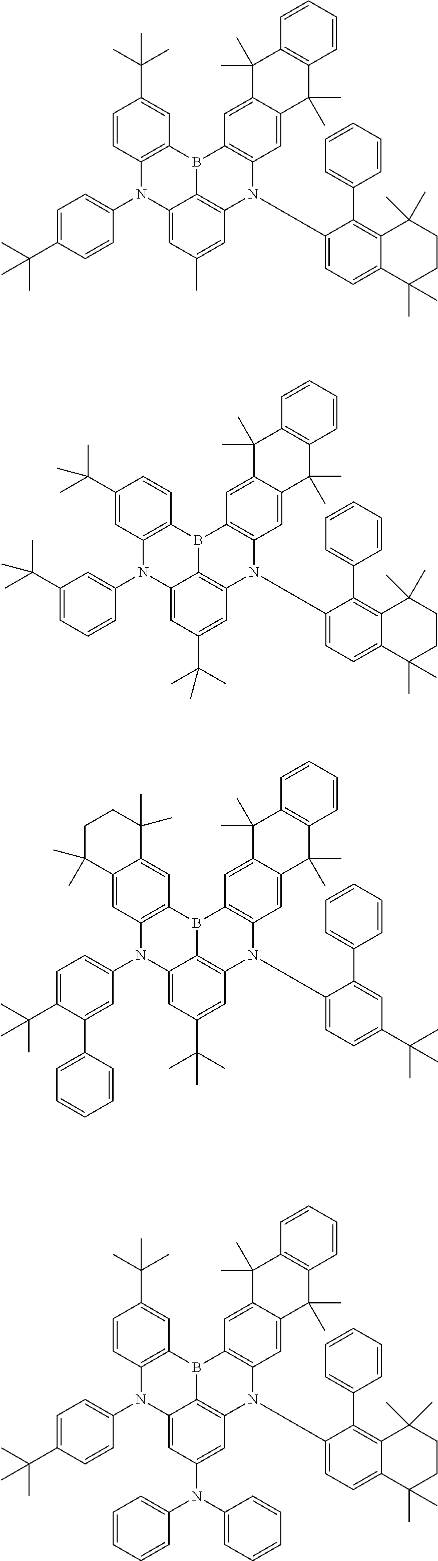

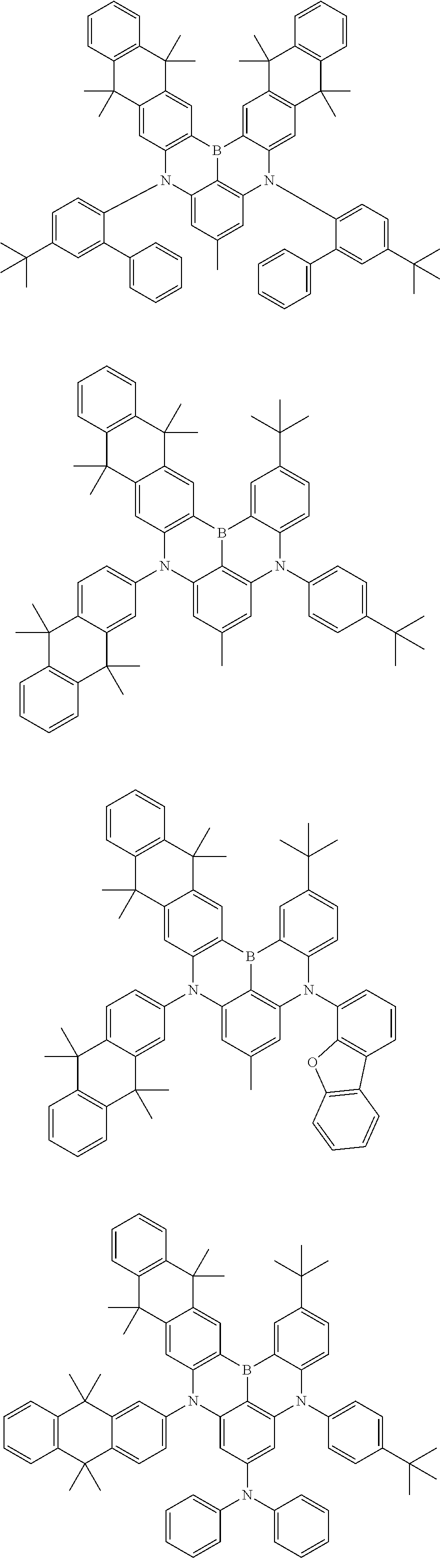

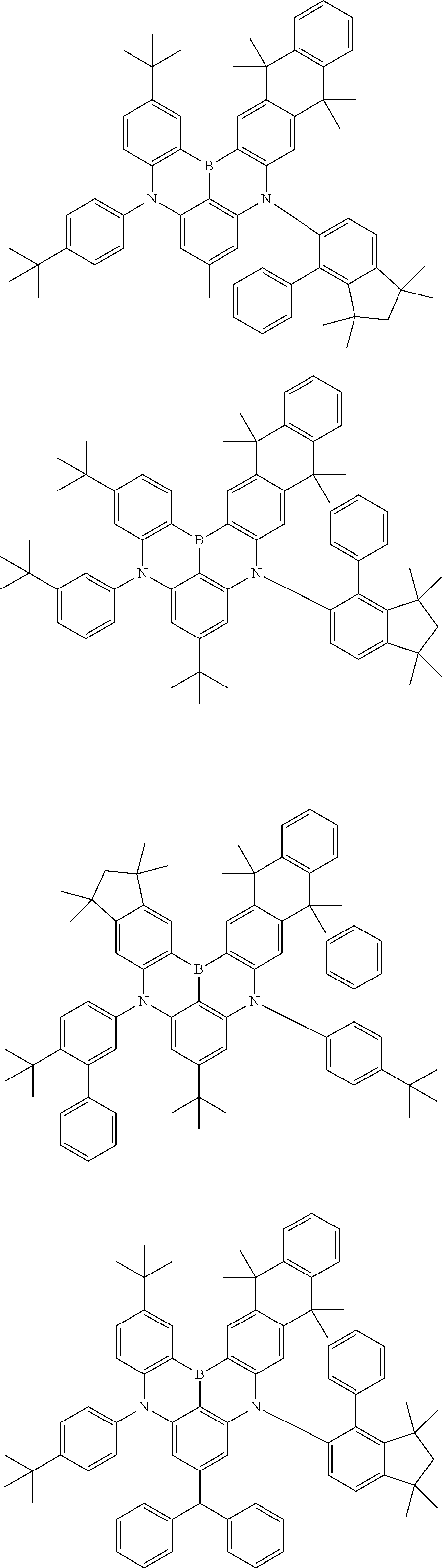

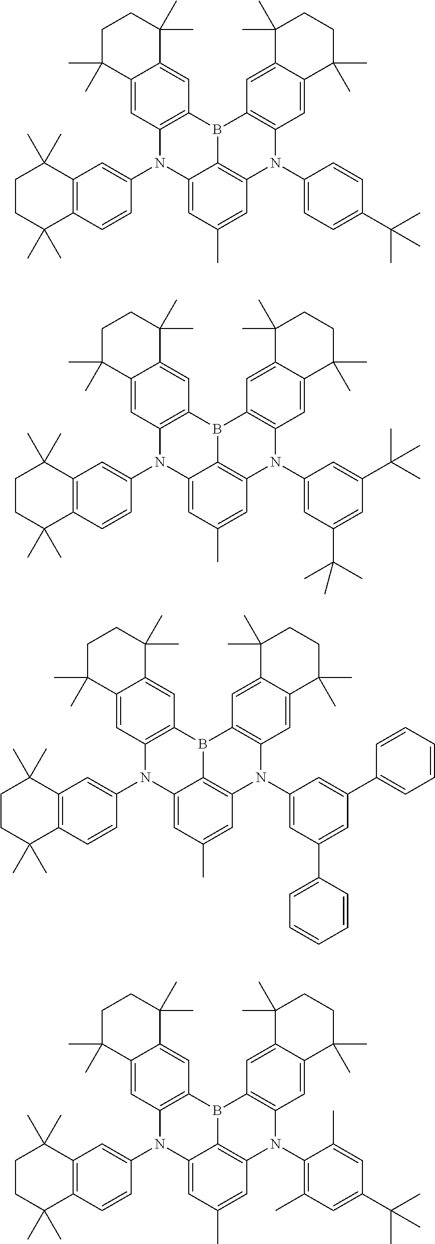 [SMILES: Cc1cc2c3c(c1)N(c1ccc4c(c1)C(C)(C)CCC4(C)C)c1cc4c(cc1B3c1cc3c(cc1N2c1ccc(C(C)(C)C)cc1)C(C)(C)CCC3(C)C)C(C)(C)CCC4(C)C ; Cc1cc2c3c(c1)N(c1ccc4c(c1)C(C)(C)CCC4(C)C)c1cc4c(cc1B3c1cc3c(cc1N2c1cc(C(C)(C)C)cc(C(C)(C)C)c1)C(C)(C)CCC3(C)C)C(C)(C)CCC4(C)C ; Cc1cc2c3c(c1)N(c1ccc4c(c1)C(C)(C)CCC4(C)C)c1cc4c(cc1B3c1cc3c(cc1N2c1cc(-c2ccccc2)cc(-c2ccccc2)c1)C(C)(C)CCC3(C)C)C(C)(C)CCC4(C)C ; Cc1cc2c3c(c1)N(c1c(C)cc(C(C)(C)C)cc1C)c1cc4c(cc1B3c1cc3c(cc1N2c1ccc2c(c1)C(C)(C)CCC2(C)C)C(C)(C)CCC3(C)C)C(C)(C)CCC4(C)C]

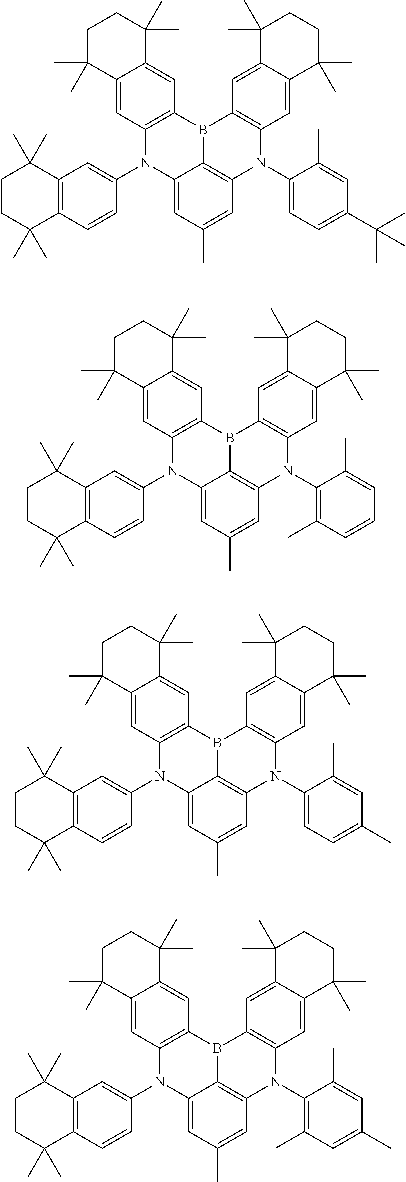 [SMILES: Cc1cc2c3c(c1)N(c1ccc(C(C)(C)C)cc1C)c1cc4c(cc1B3c1cc3c(cc1N2c1ccc2c(c1)C(C)(C)CCC2(C)C)C(C)(C)CCC3(C)C)C(C)(C)CCC4(C)C ; Cc1cc2c3c(c1)N(c1c(C)cccc1C)c1cc4c(cc1B3c1cc3c(cc1N2c1ccc2c(c1)C(C)(C)CCC2(C)C)C(C)(C)CCC3(C)C)C(C)(C)CCC4(C)C ; Cc1ccc(N2c3cc4c(cc3B3c5cc6c(cc5N(c5ccc7c(c5)C(C)(C)CCC7(C)C)c5cc(C)cc2c53)C(C)(C)CCC6(C)C)C(C)(C)CCC4(C)C)c(C)c1 ; Cc1cc(C)c(N2c3cc4c(cc3B3c5cc6c(cc5N(c5ccc7c(c5)C(C)(C)CCC7(C)C)c5cc(C)cc2c53)C(C)(C)CCC6(C)C)C(C)(C)CCC4(C)C)c(C)c1]

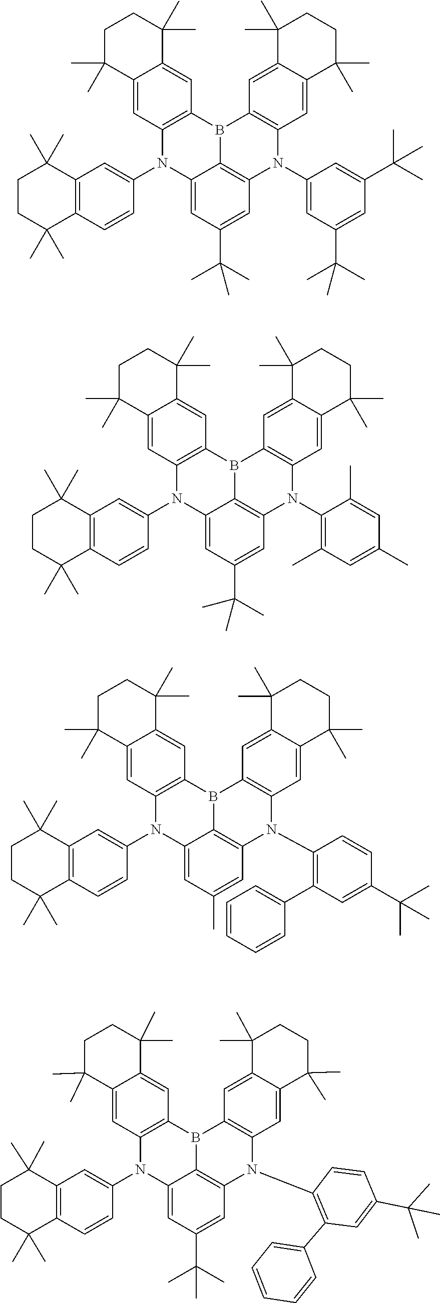

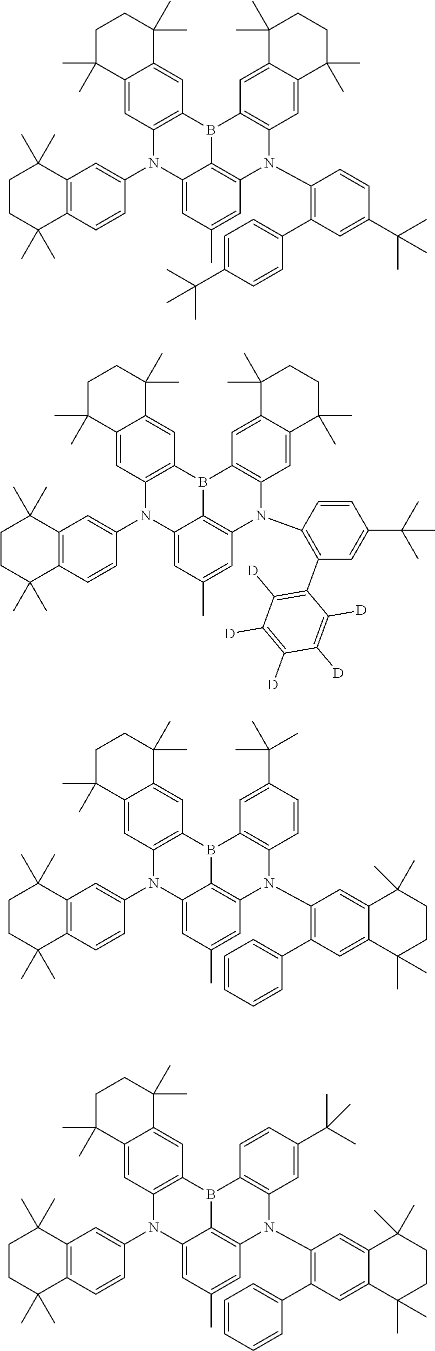

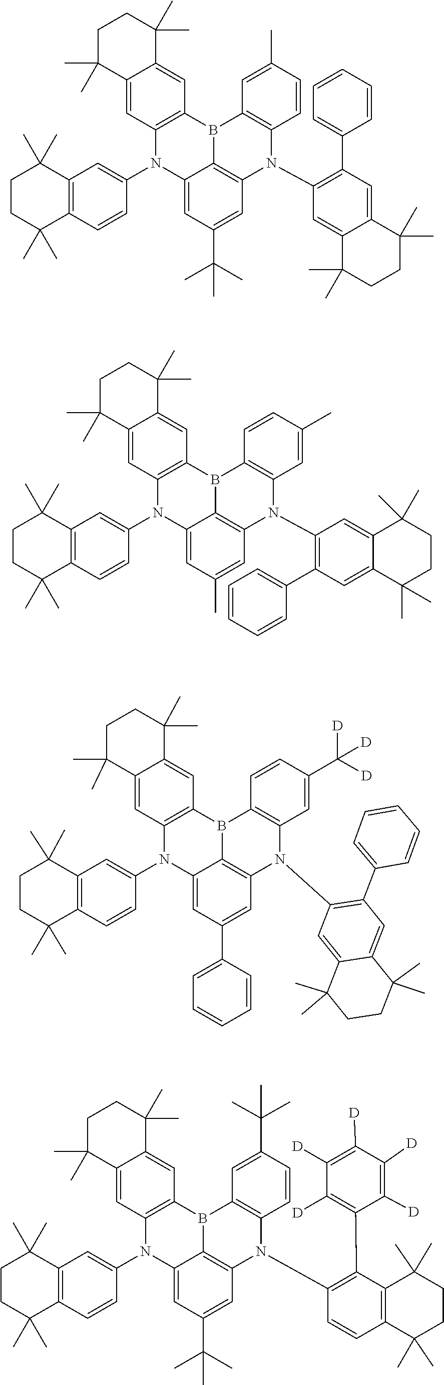

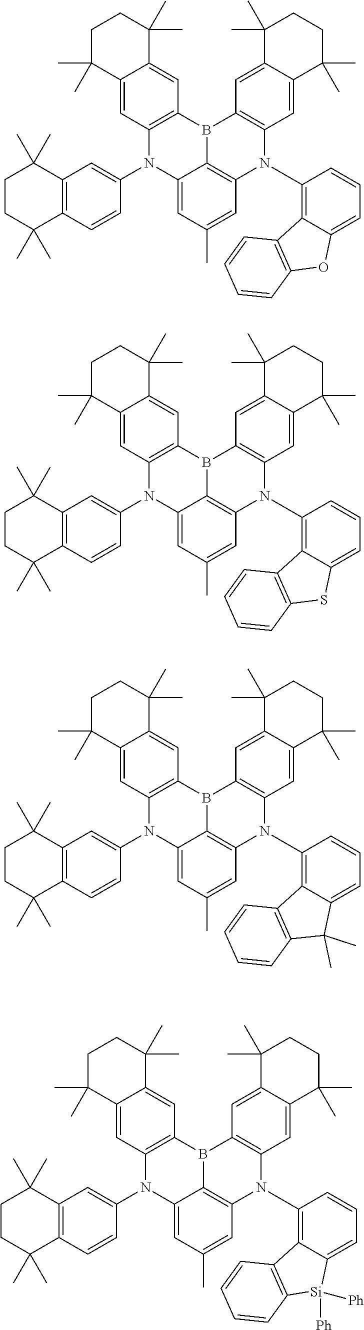

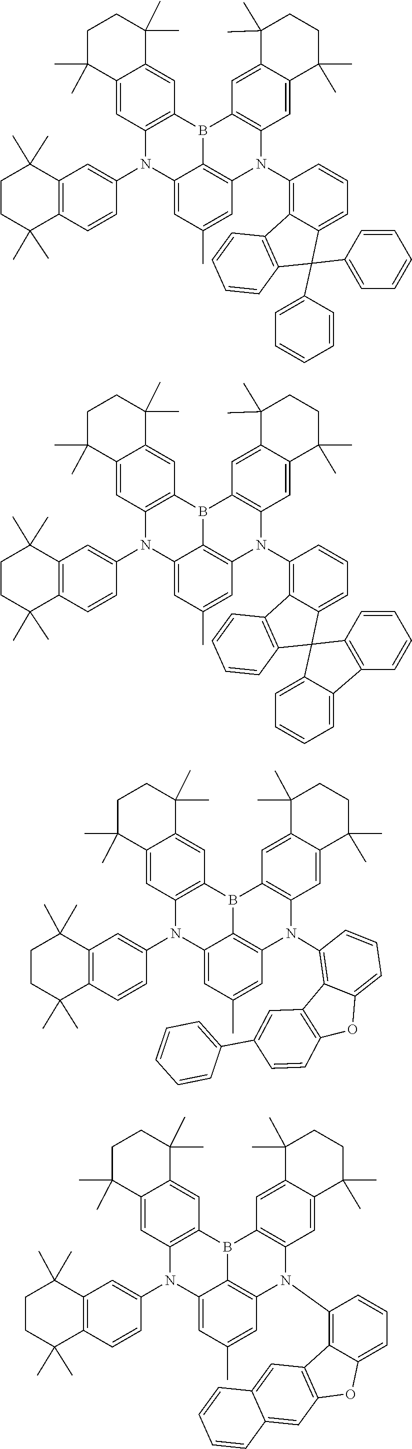

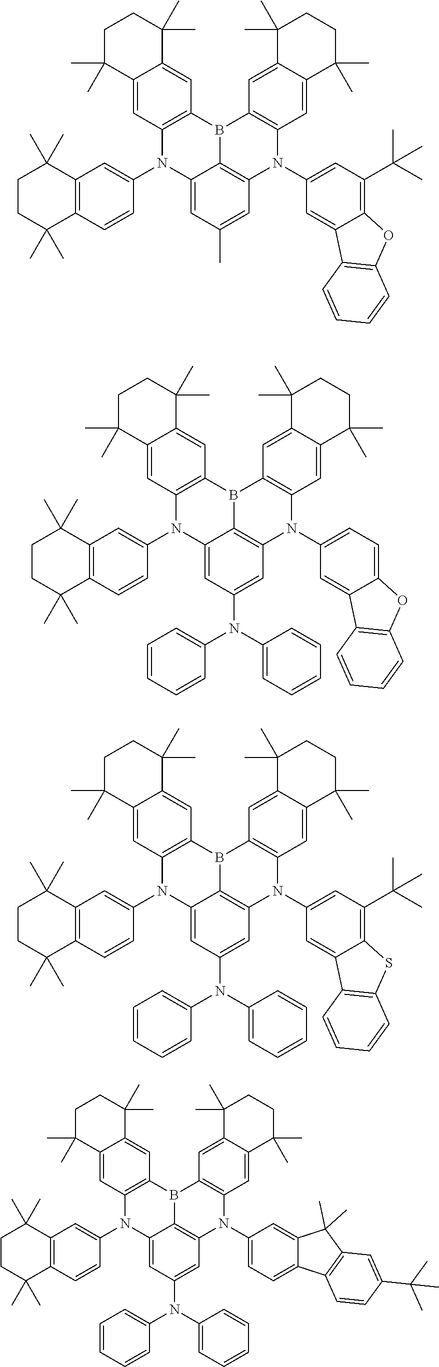

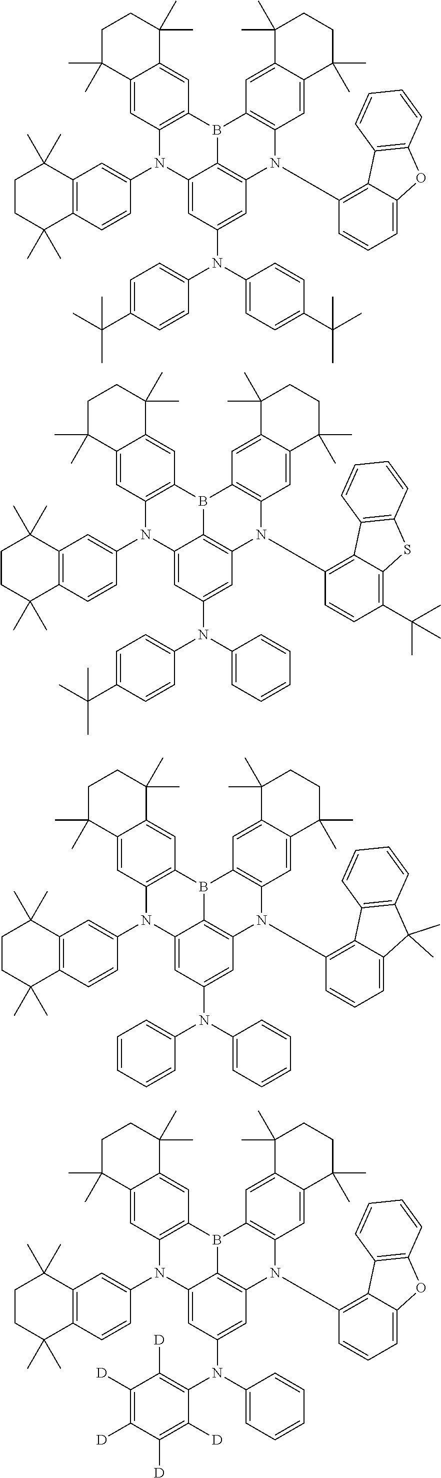

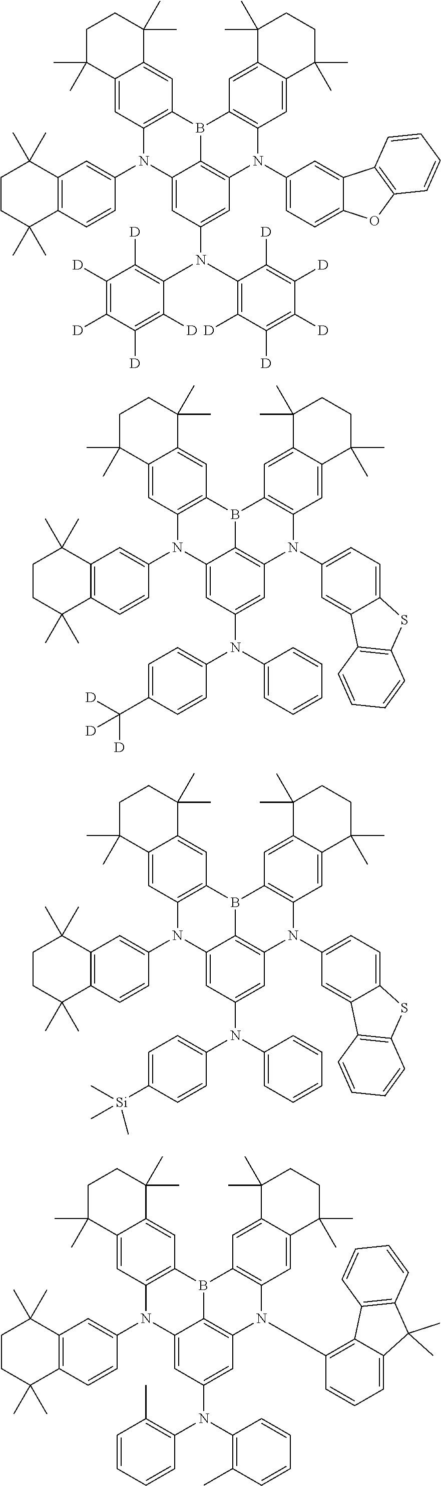

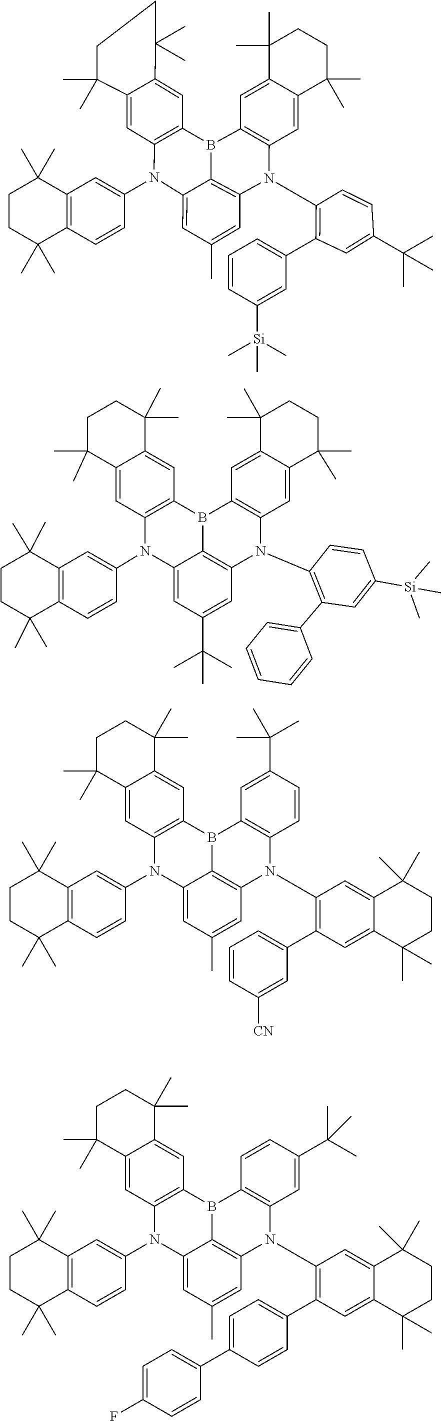

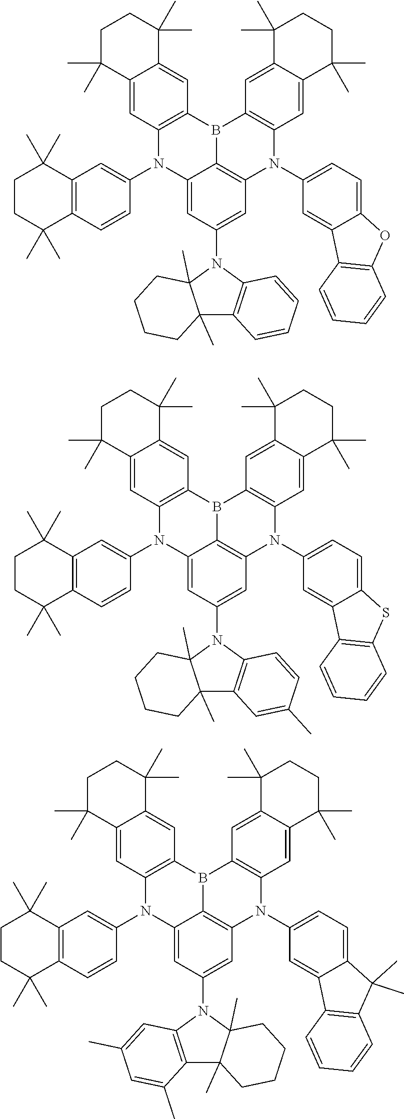

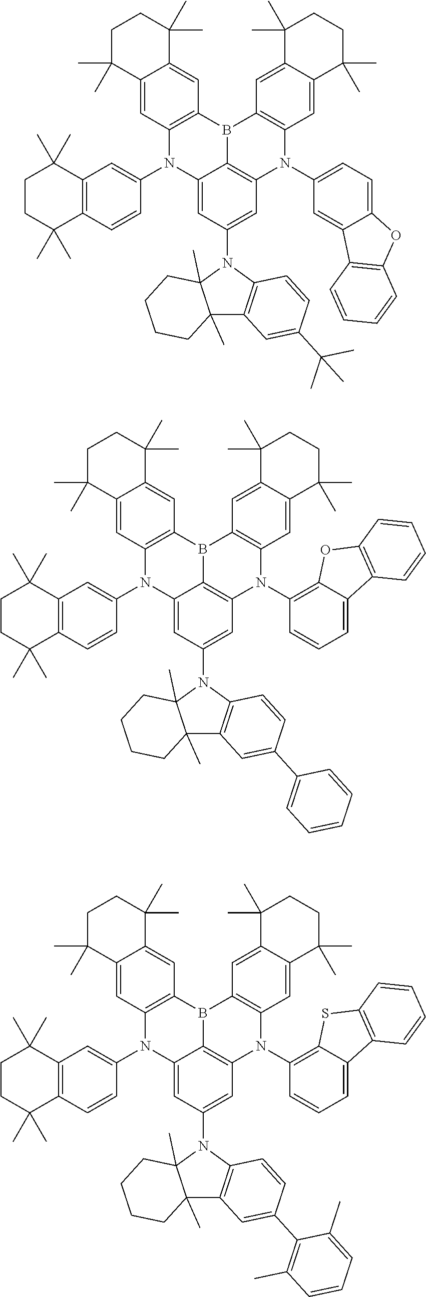 [SMILES: CC(C)(C)c1ccc2c(c1)C1(C)CCCCC1(C)N2c1cc2c3c(c1)N(c1ccc4oc5ccccc5c4c1)c1cc4c(cc1B3c1cc3c(cc1N2c1ccc2c(c1)C(C)(C)CCC2(C)C)C(C)(C)CCC3(C)C)C(C)(C)CCC4(C)C ; CC1(C)CCC(C)(C)c2cc(N3c4cc5c(cc4B4c6cc7c(cc6N(c6cccc8c6oc6ccccc68)c6cc(N8c9ccc(-c%10ccccc%10)cc9C9(C)CCCCC89C)cc3c64)C(C)(C)CCC7(C)C)C(C)(C)CCC5(C)C)ccc21 ; Cc1cccc(C)c1-c1ccc2c(c1)C1(C)CCCCC1(C)N2c1cc2c3c(c1)N(c1cccc4c1sc1ccccc14)c1cc4c(cc1B3c1cc3c(cc1N2c1ccc2c(c1)C(C)(C)CCC2(C)C)C(C)(C)CCC3(C)C)C(C)(C)CCC4(C)C]

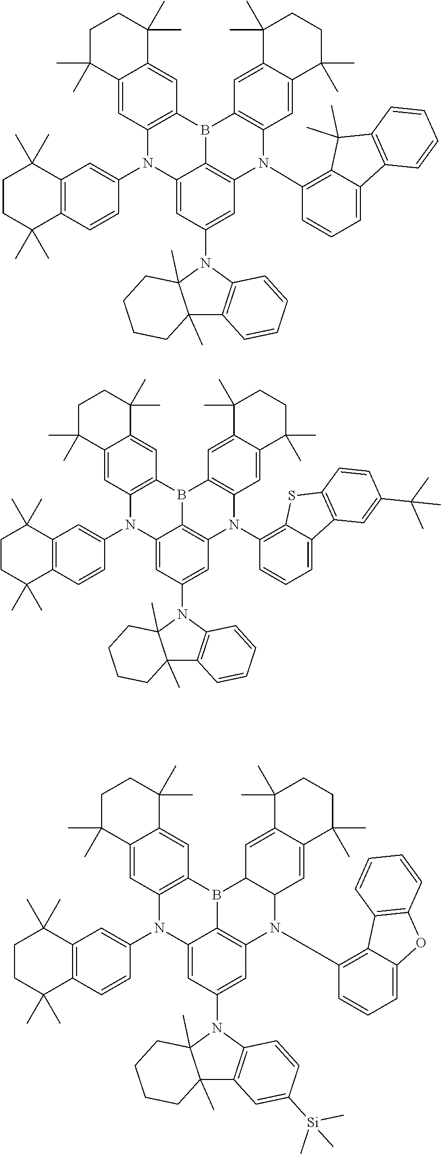

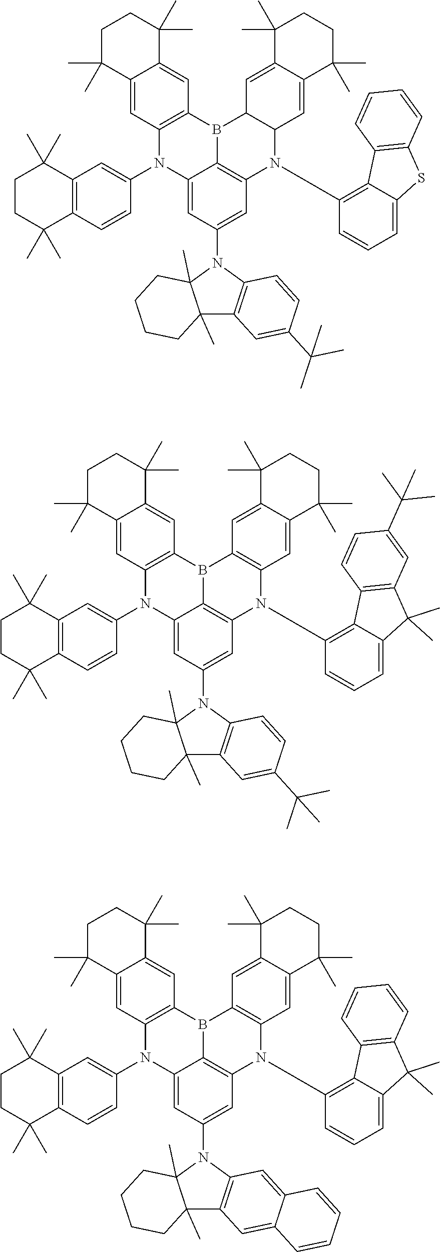 [SMILES: CC1(C)CCC(C)(C)C2=CC3C(C=C21)B1c2cc4c(cc2N(c2ccc5c(c2)C(C)(C)CCC5(C)C)c2cc(N5c6ccc(C(C)(C)C)cc6C6(C)CCCCC56C)cc(c21)N3c1cccc2sc3ccccc3c12)C(C)(C)CCC4(C)C ; CC(C)(C)c1ccc2c(c1)C(C)(C)c1cccc(N3c4cc5c(cc4B4c6cc7c(cc6N(c6ccc8c(c6)C(C)(C)CCC8(C)C)c6cc(N8c9ccc(C(C)(C)C)cc9C9(C)CCCCC89C)cc3c64)C(C)(C)CCC7(C)C)C(C)(C)CCC5(C)C)c1-2 ; CC1(C)CCC(C)(C)c2cc(N3c4cc5c(cc4B4c6cc7c(cc6N(c6cccc8c6-c6ccccc6C8(C)C)c6cc(N8c9cc%10ccccc%10cc9C9(C)CCCCC89C)cc3c64)C(C)(C)CCC7(C)C)C(C)(C)CCC5(C)C)ccc21]

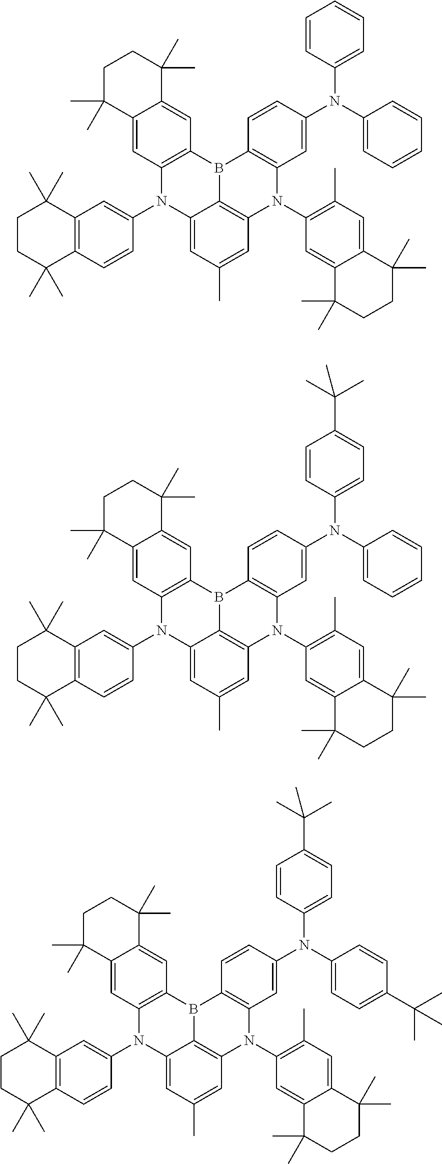

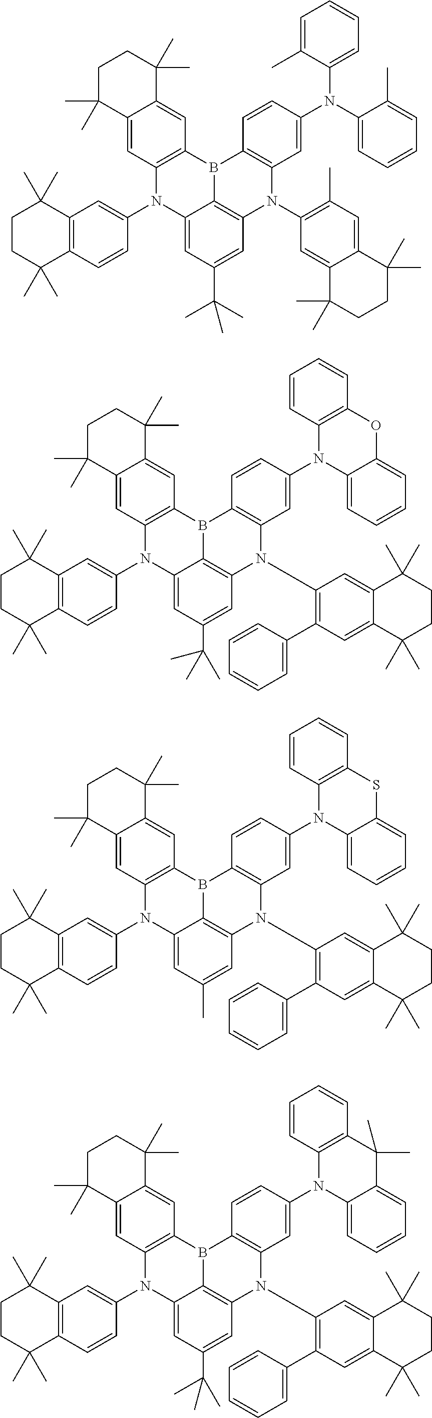

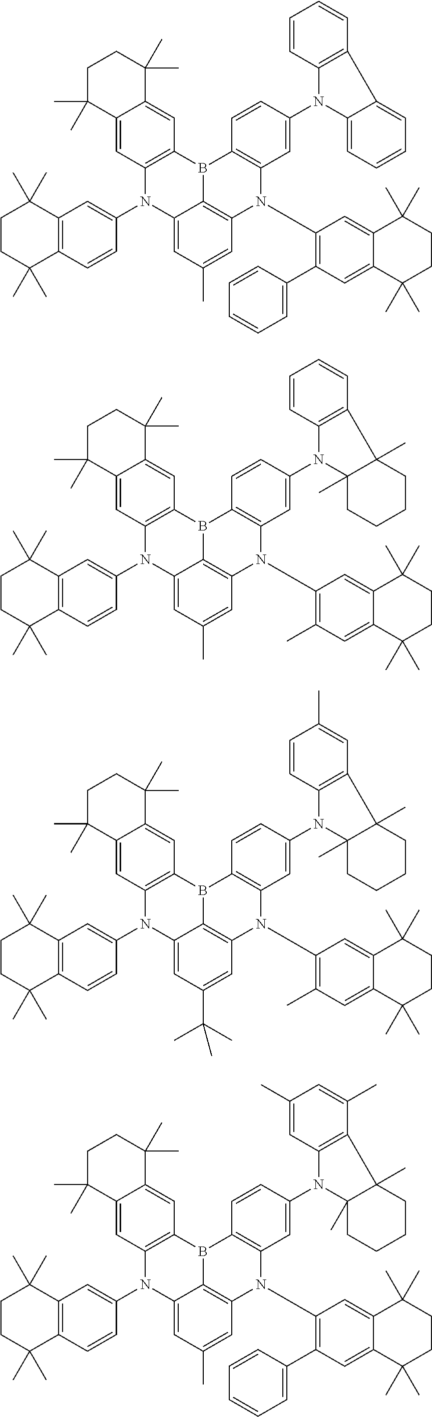

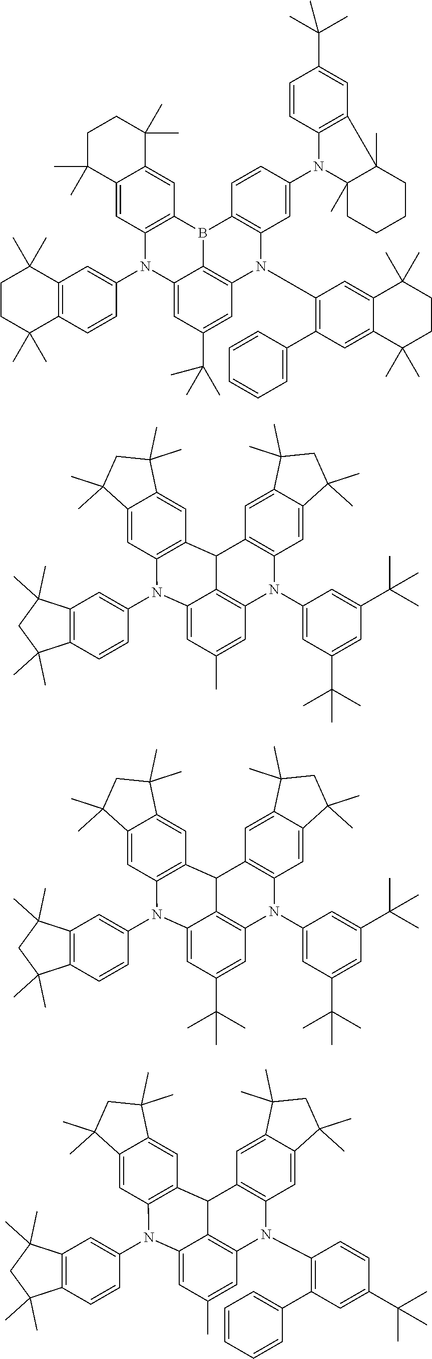

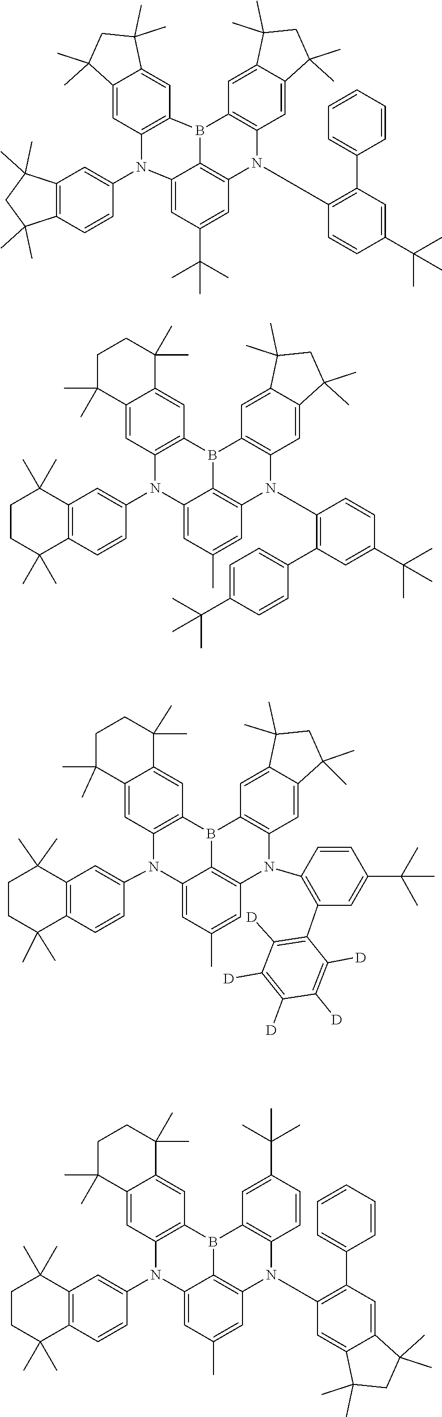

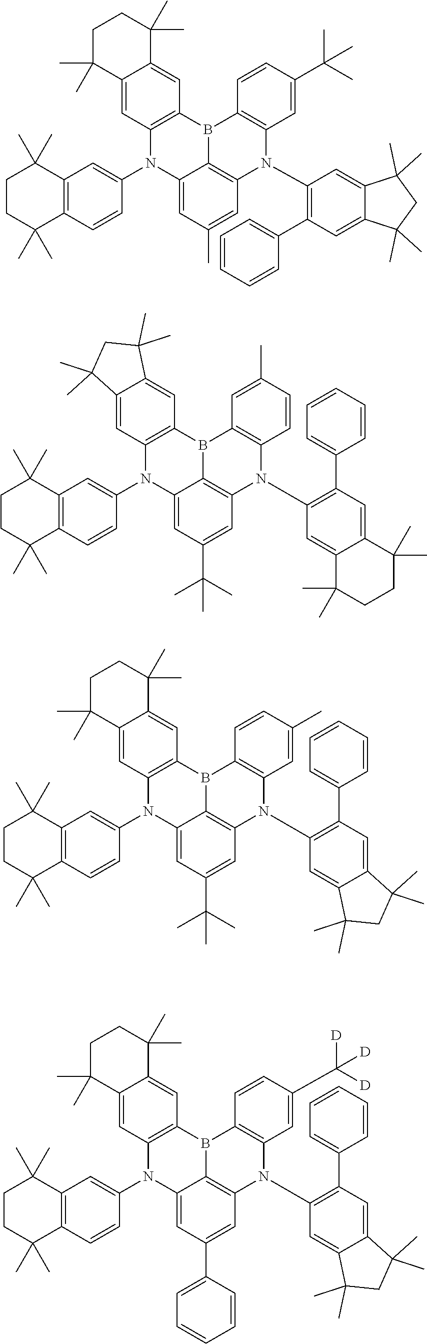

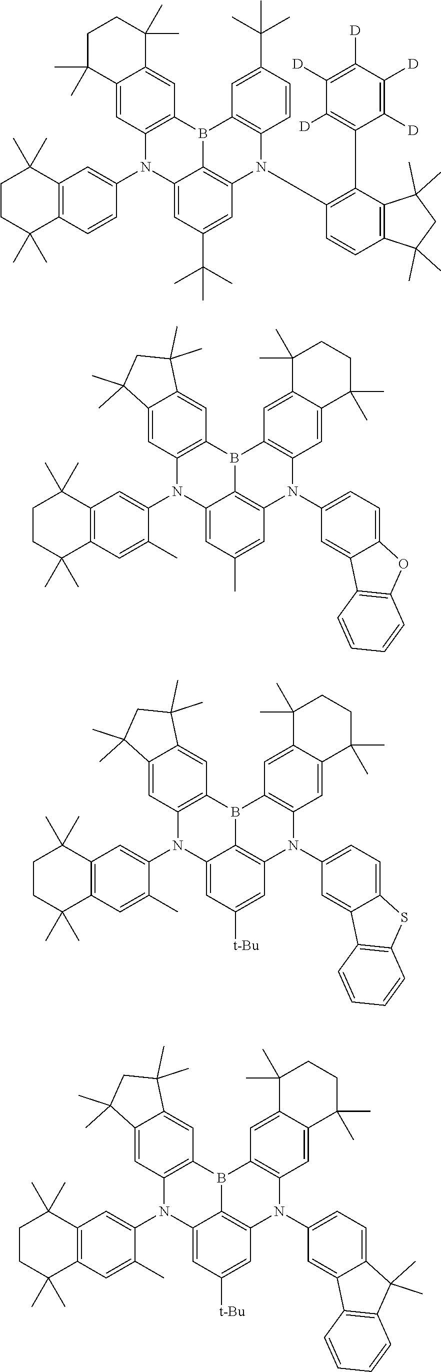

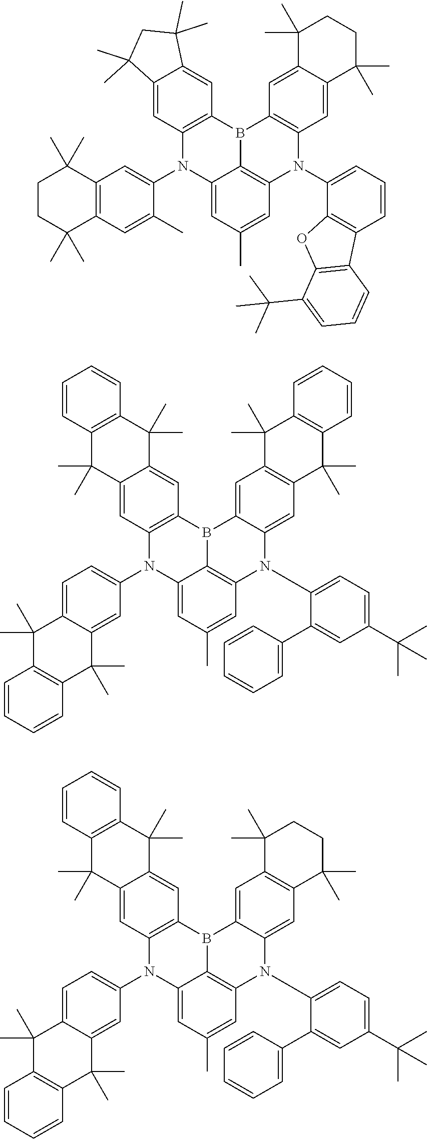

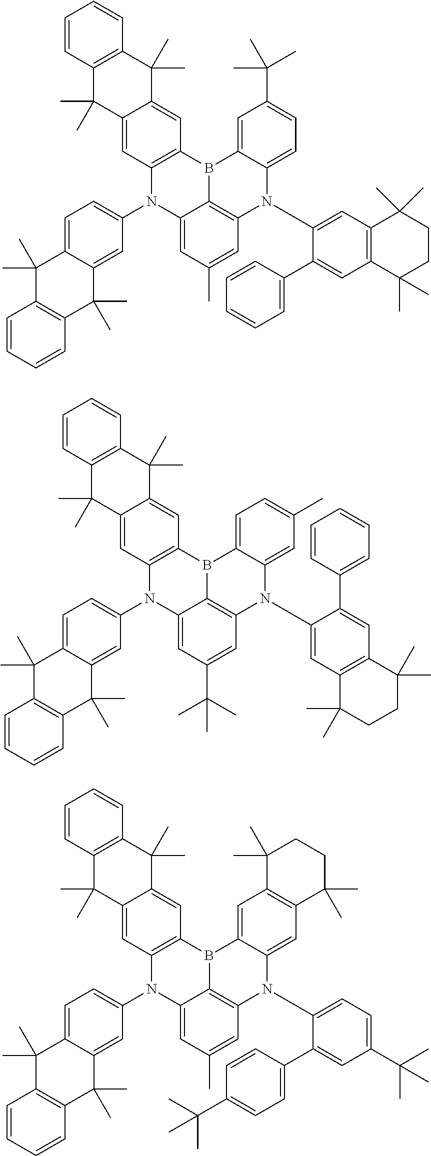

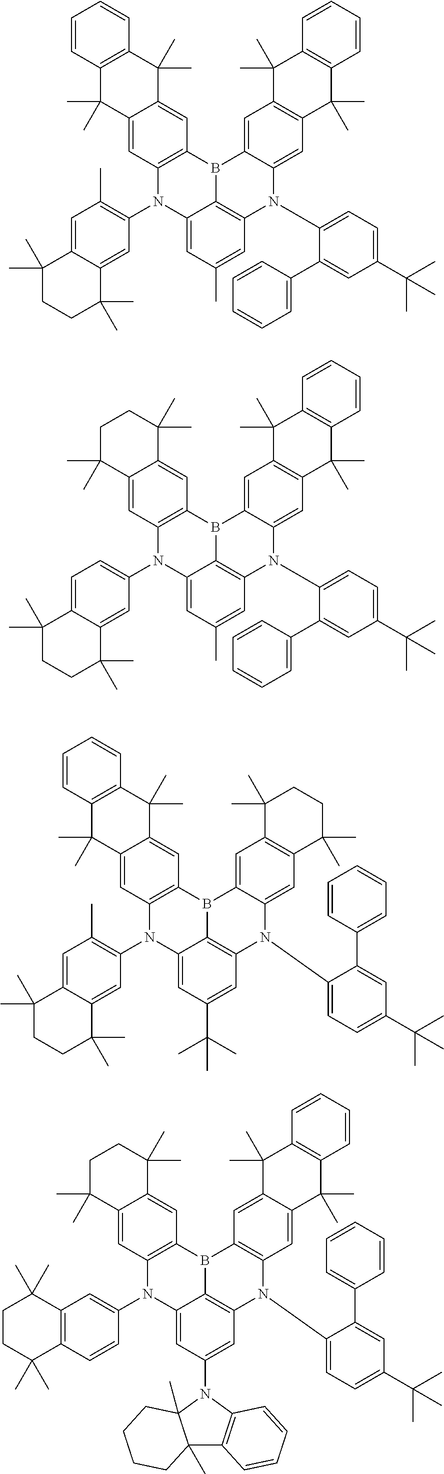

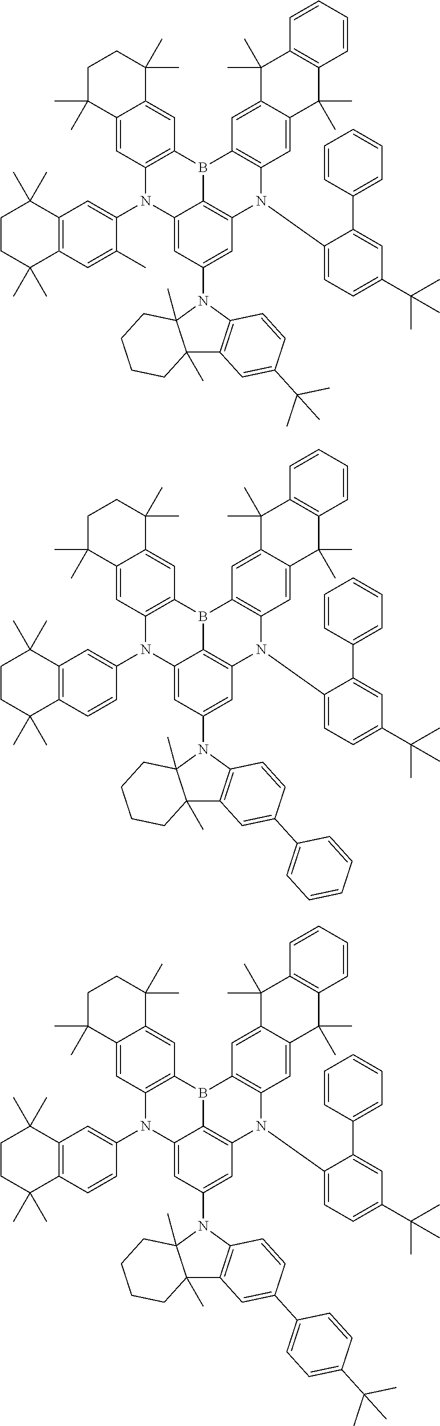

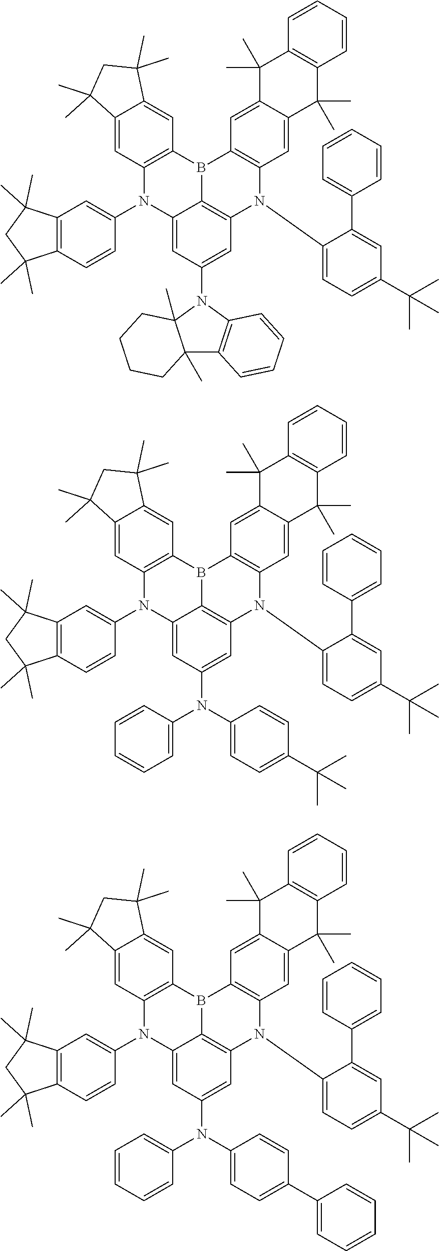

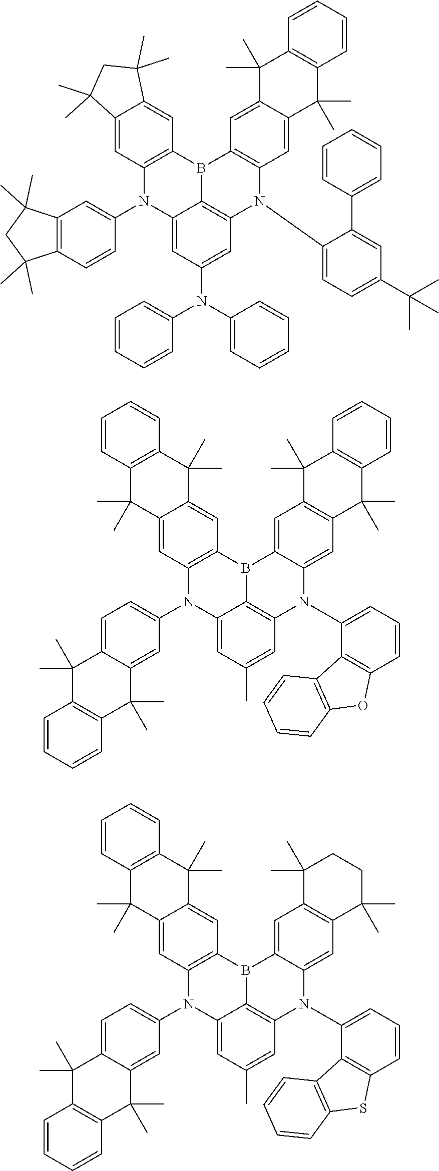

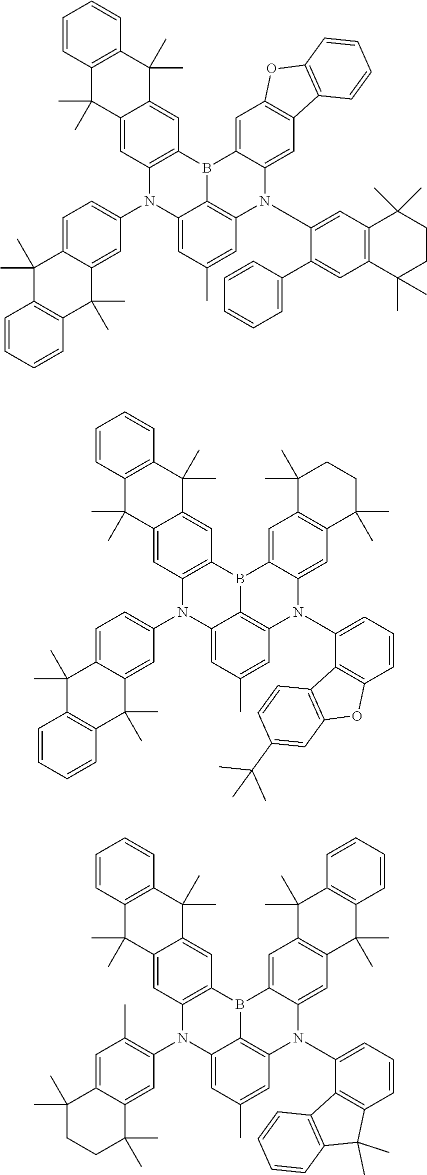

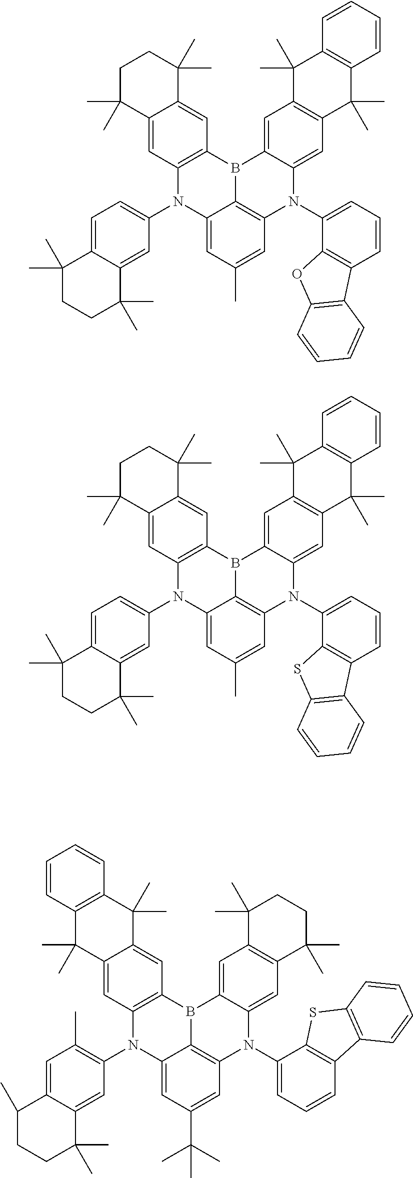

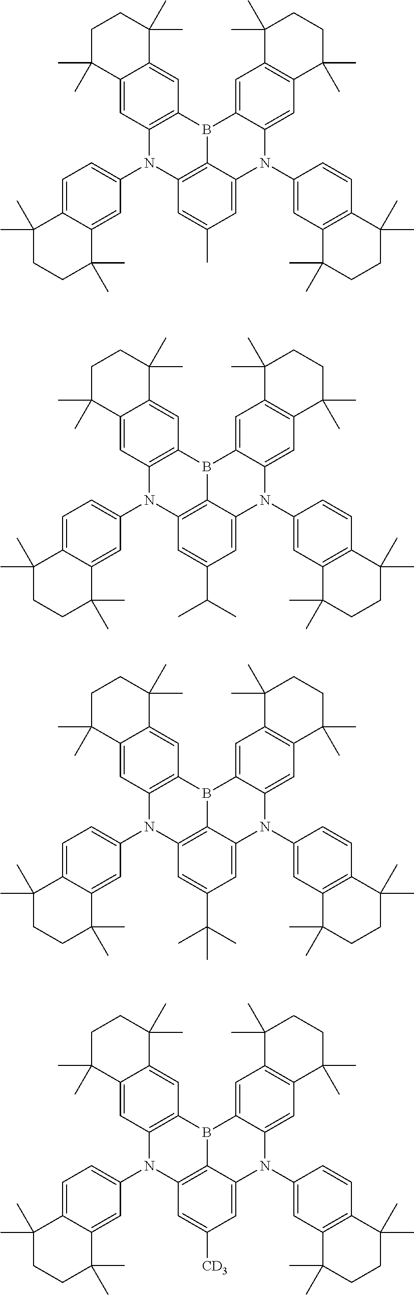

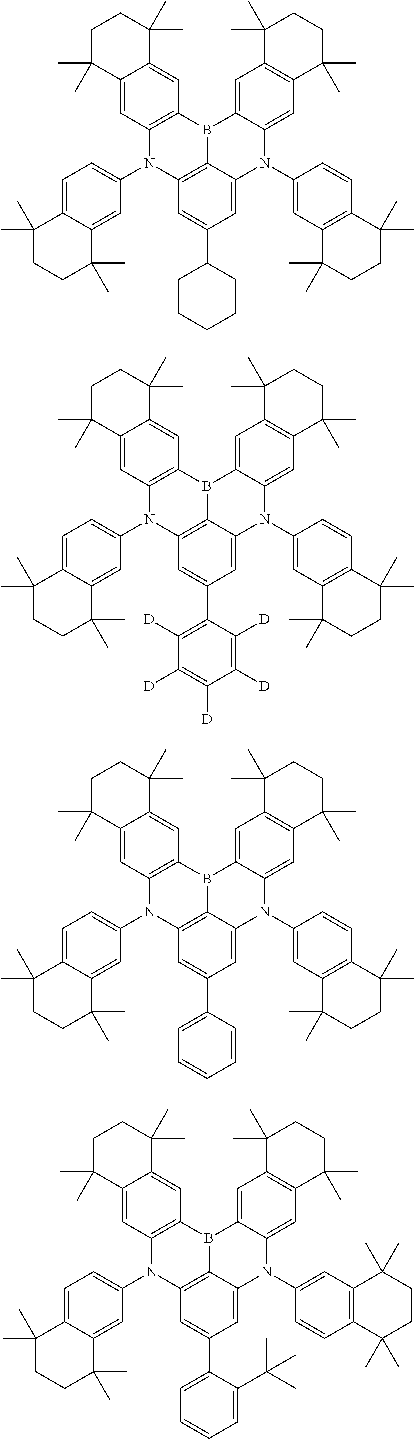

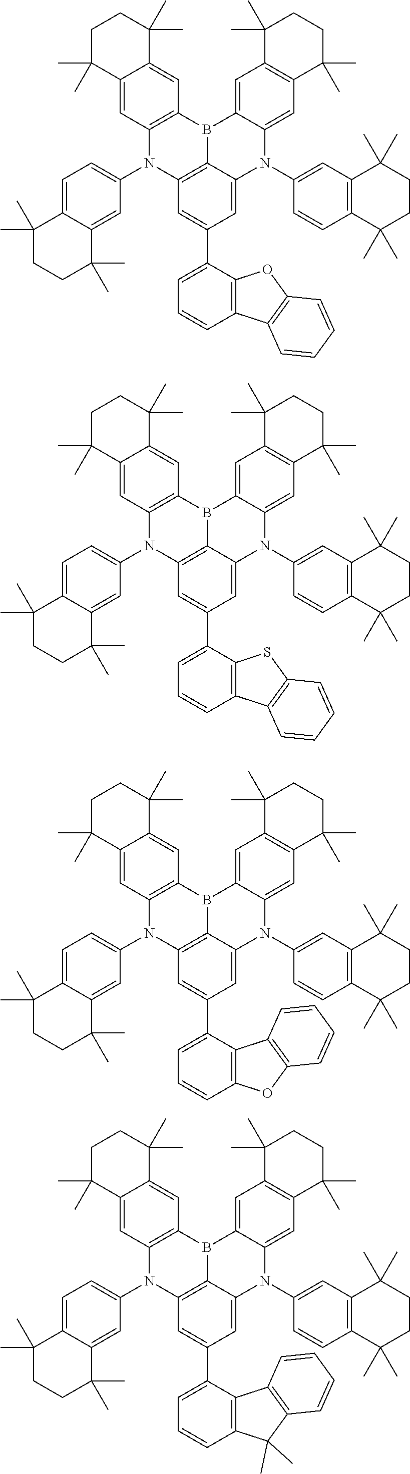

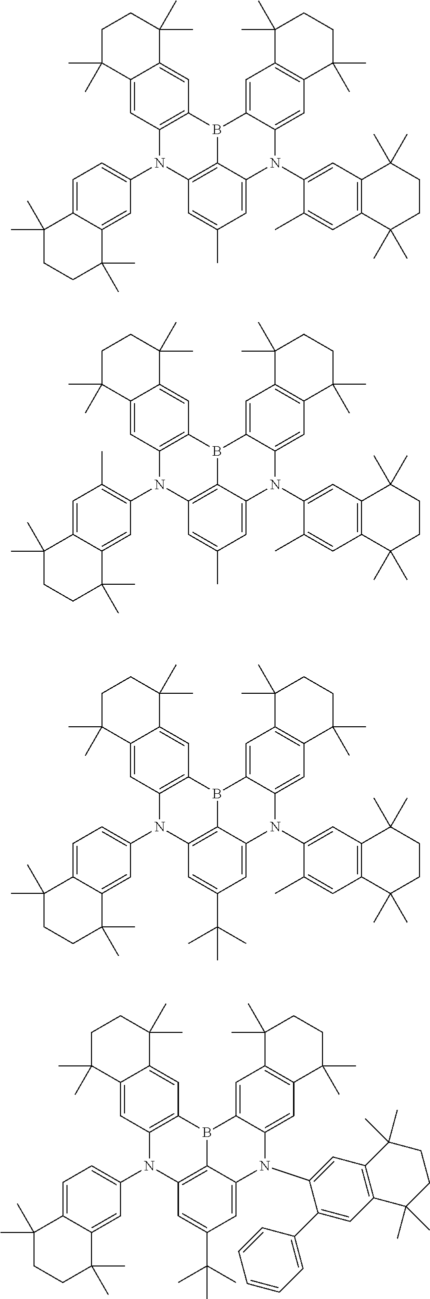

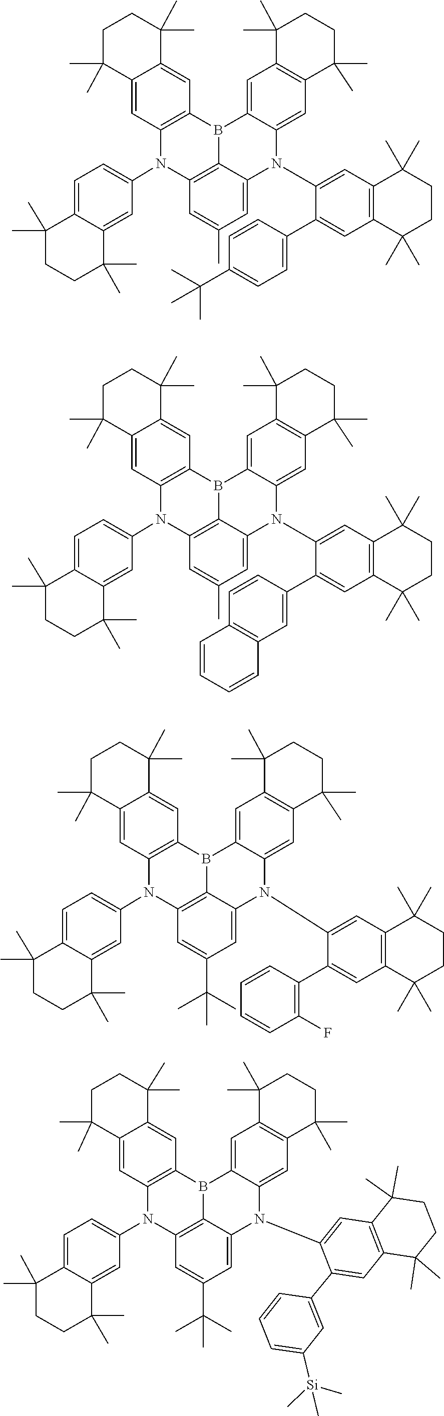

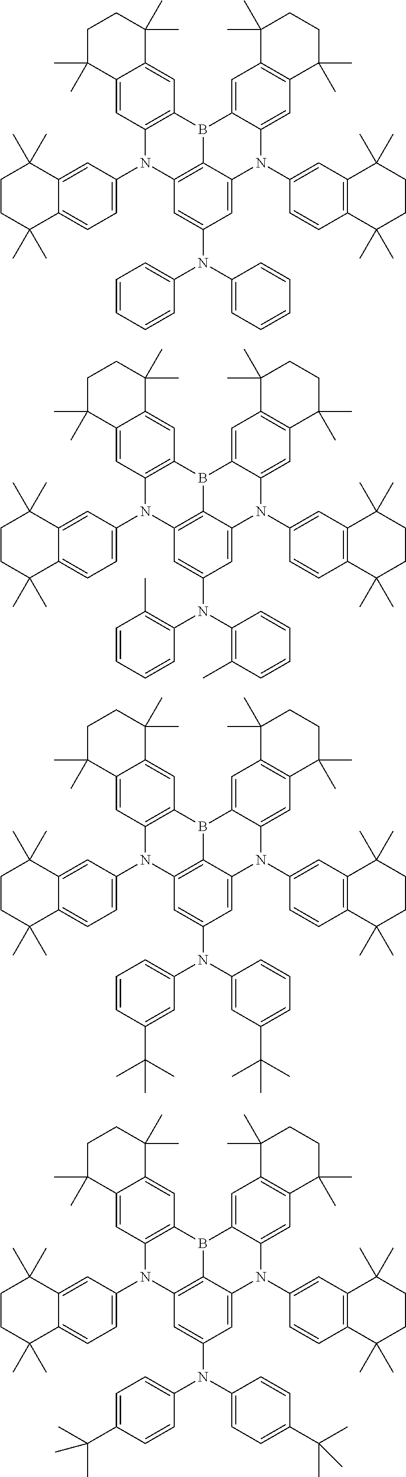

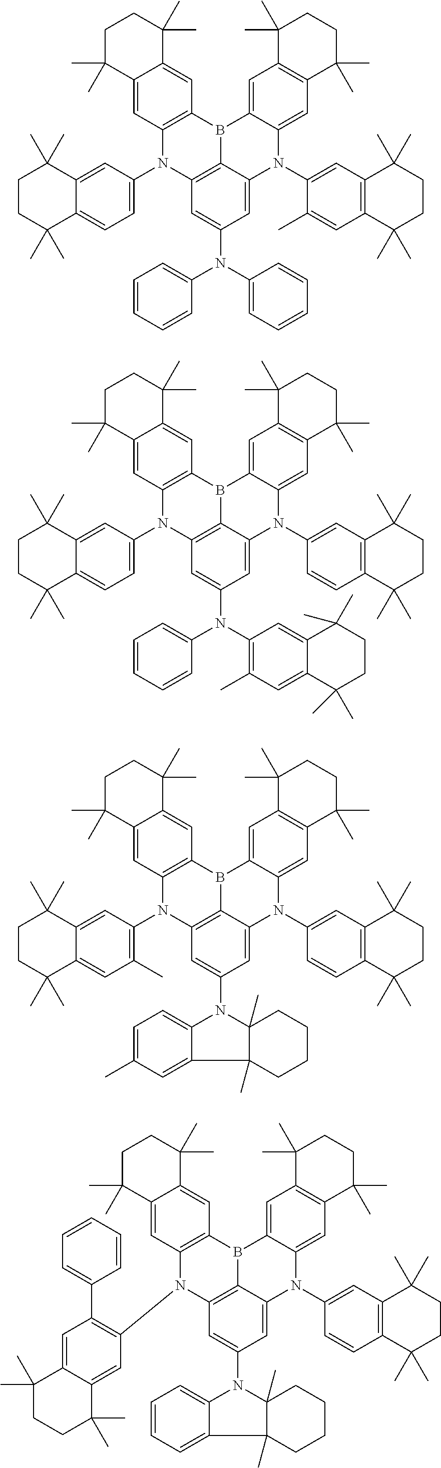

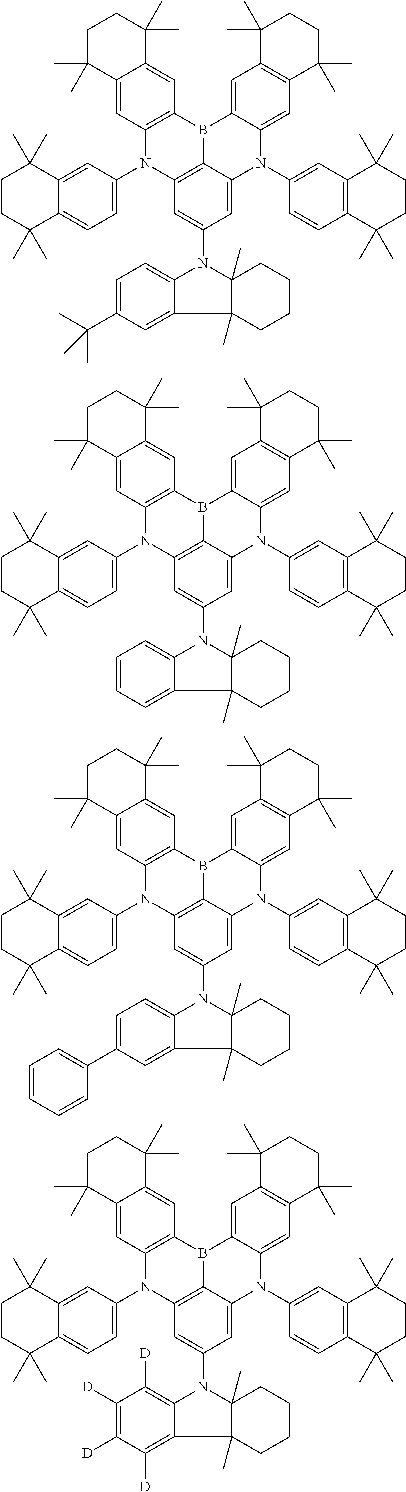

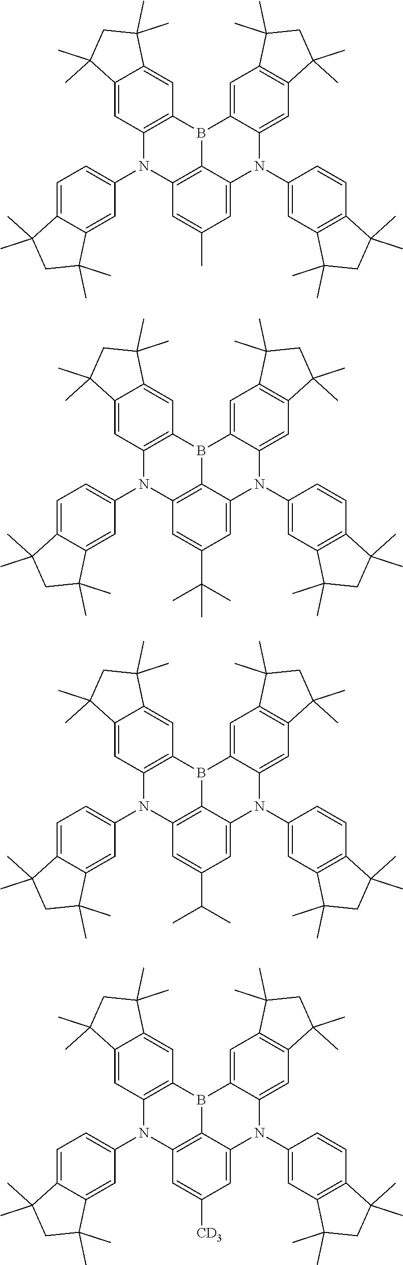

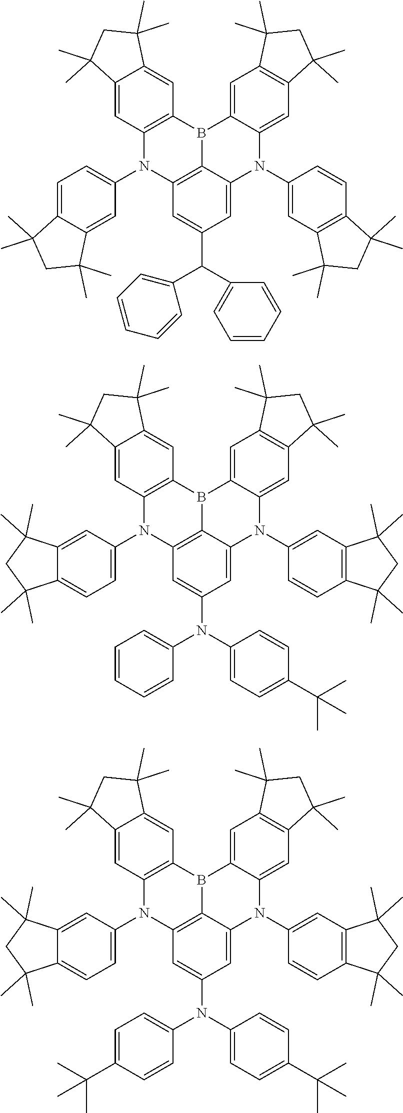

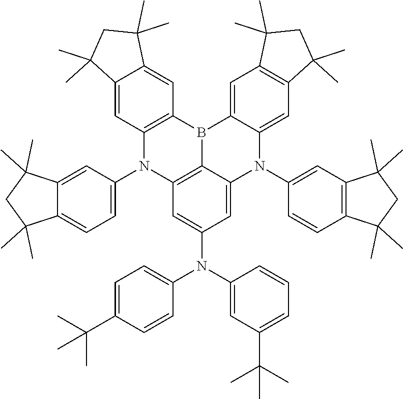

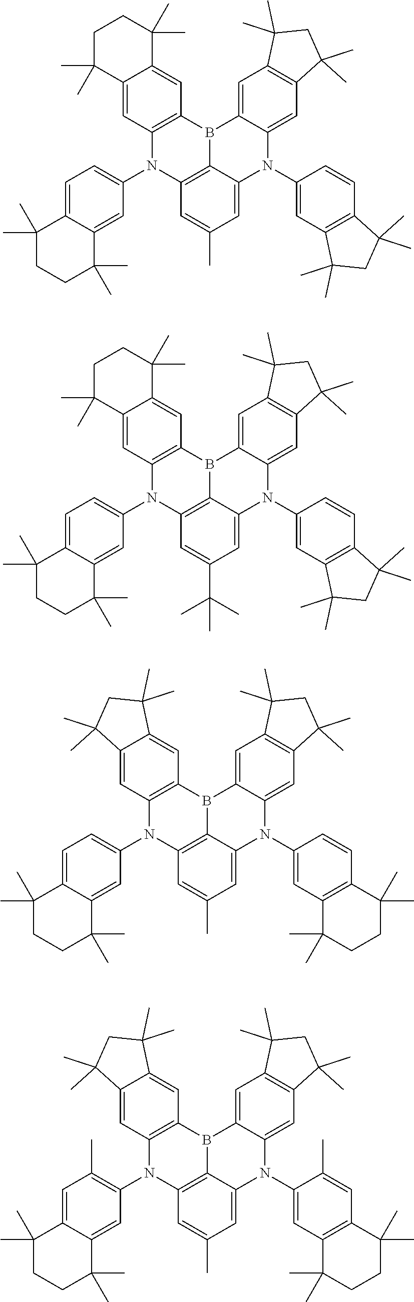

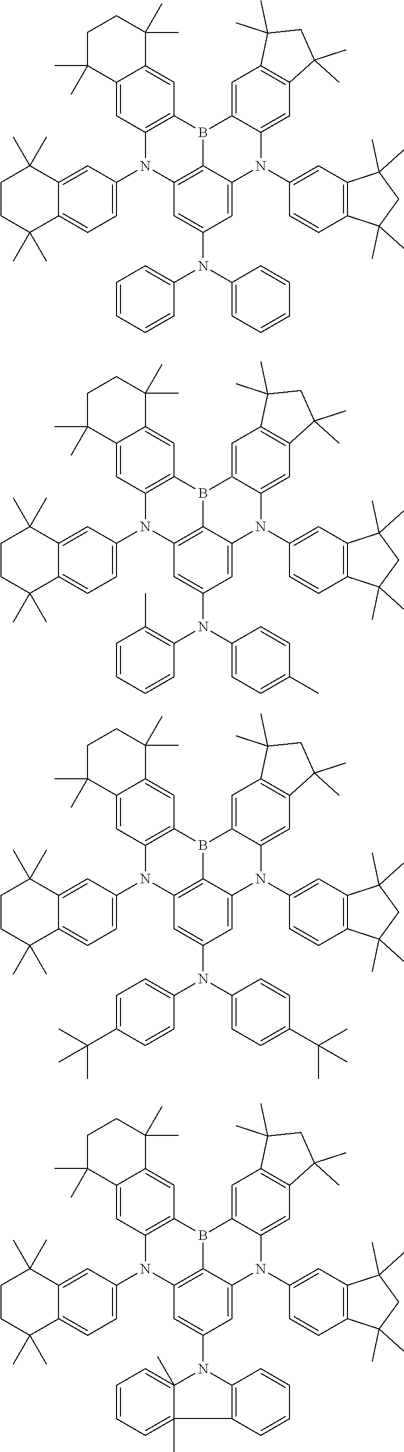

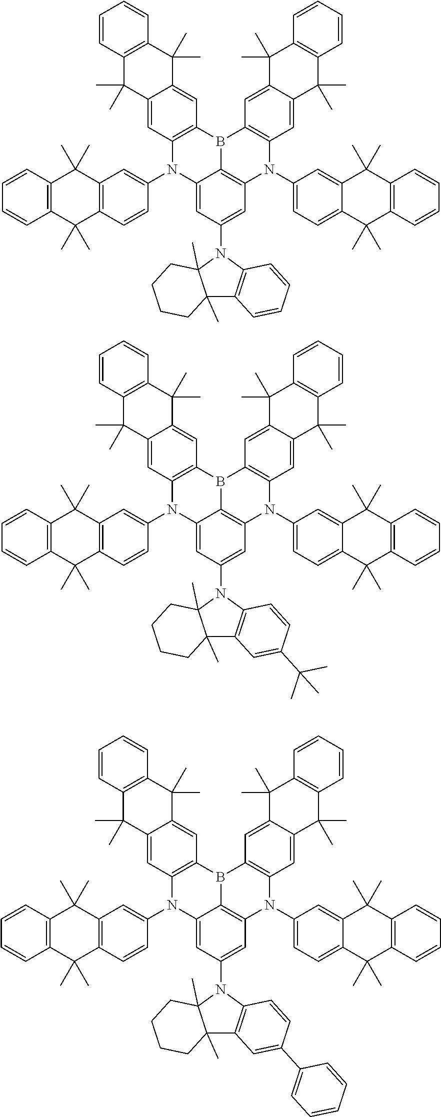

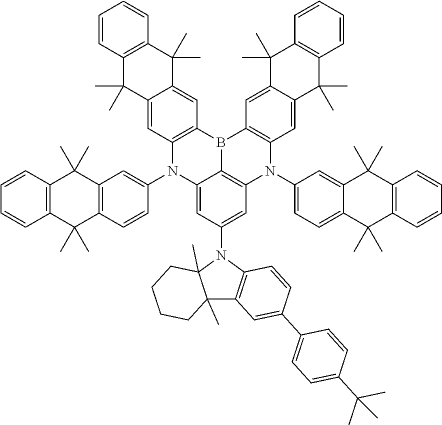 [SMILES: CC(C)(C)c1ccc(-c2ccc3c(c2)C2(C)CCCCC2(C)N3c2cc3c4c(c2)N(c2ccc5c(c2)C(C)(C)c2ccccc2C5(C)C)c2cc5c(cc2B4c2cc4c(cc2N3c2ccc3c(c2)C(C)(C)c2ccccc2C3(C)C)C(C)(C)c2ccccc2C4(C)C)C(C)(C)c2ccccc2C5(C)C)cc1]

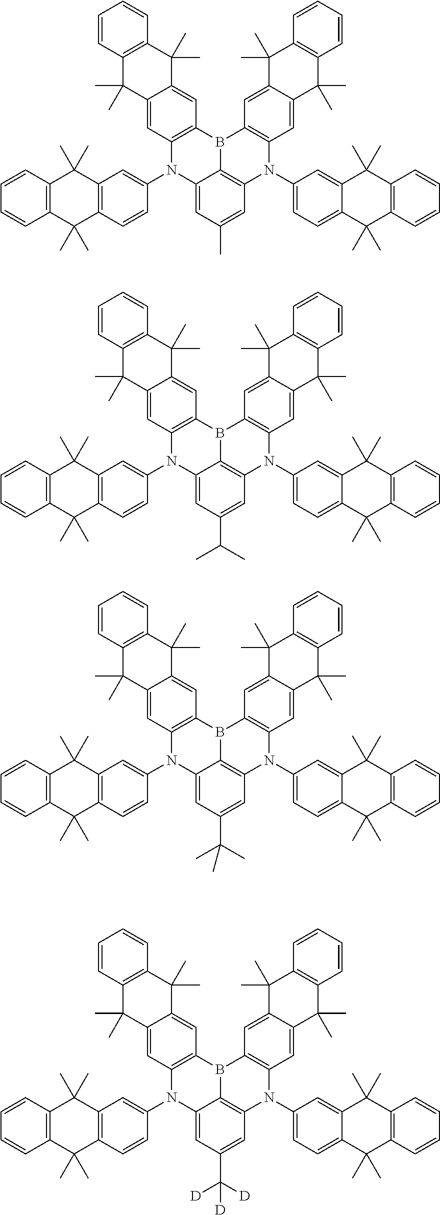

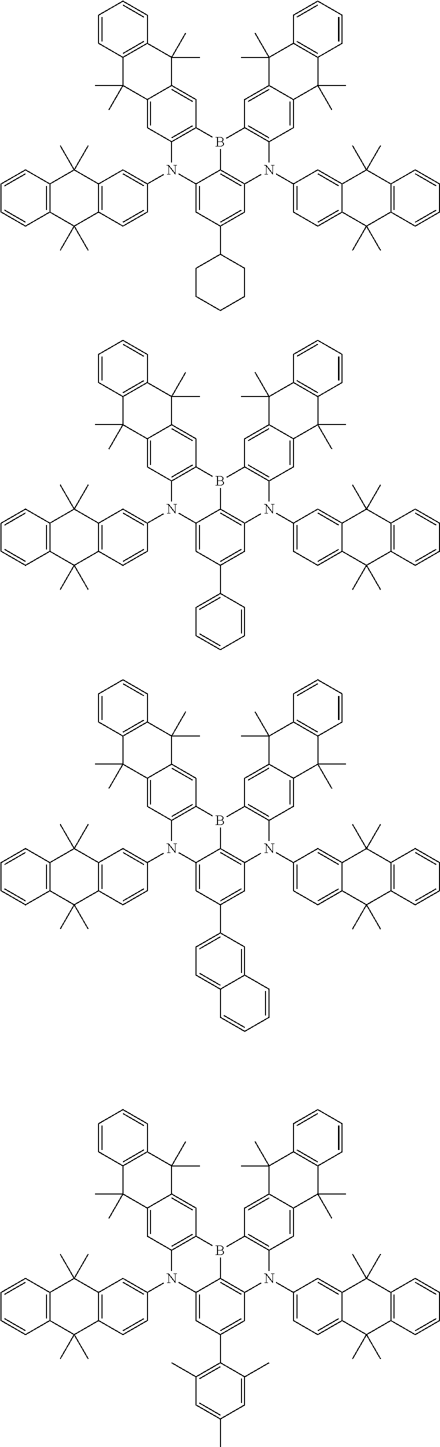

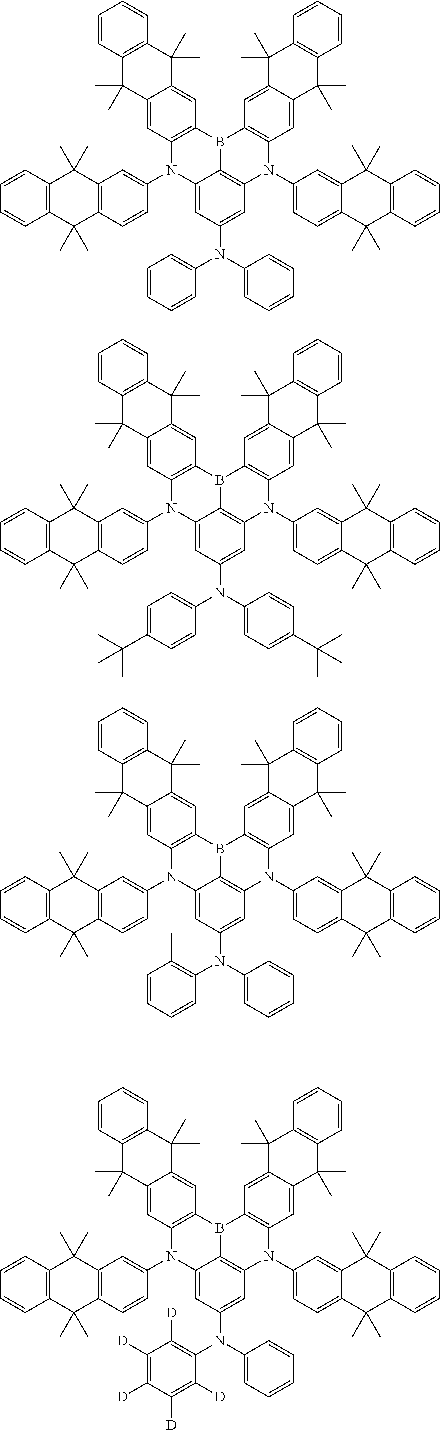

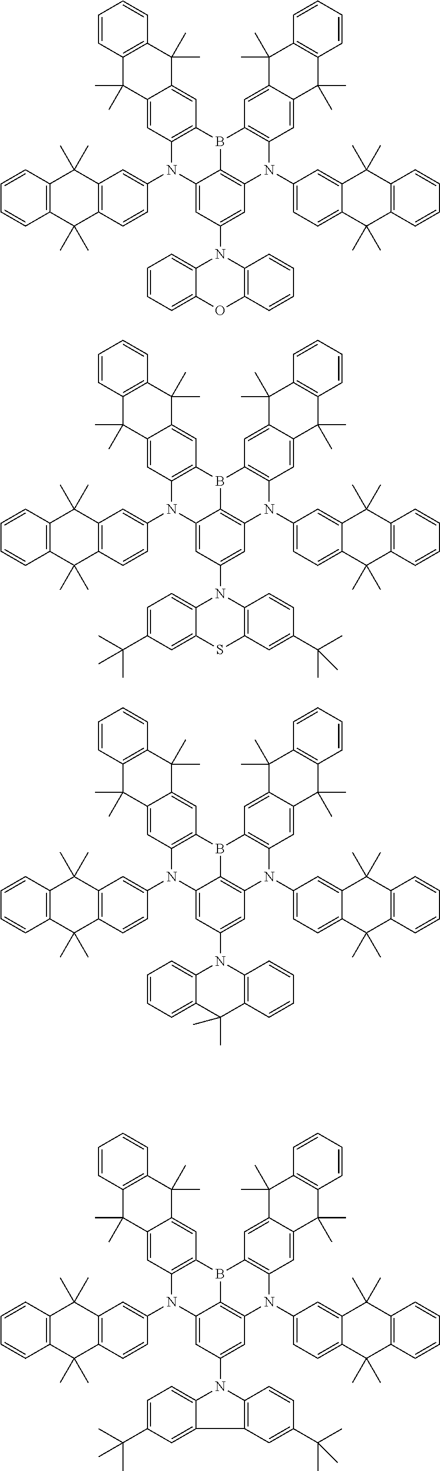

Hereinafter, Chemical Formula 2 will be described in detail.

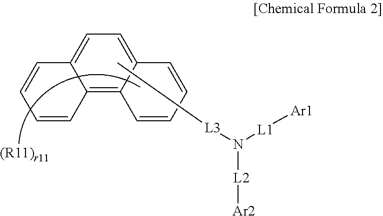

wherein in Chemical Formula 2:

L1 to L3 are the same as or different from each other, and are each independently a direct bond, a substituted or unsubstituted arylene group, or a substituted or unsubstituted divalent heterocyclic group;

Ar1 and Ar2 are the same as or different from each other, and are each independently one selected from the group consisting of a substituted or unsubstituted aromatic hydrocarbon ring, a substituted or unsubstituted aliphatic hydrocarbon ring, and a substituted or unsubstituted aromatic hetero ring, or a ring in which two or more rings selected from the above group are fused;

R11 is hydrogen, deuterium, a cyano group, a halogen group, a substituted or unsubstituted alkyl group, a substituted or unsubstituted alkoxy group, a substituted or unsubstituted alkylthio group, a substituted or unsubstituted cycloalkyl group, a substituted or unsubstituted silyl group, a substituted or unsubstituted aryl group, a substituted or unsubstituted aryloxy group, a substituted or unsubstituted arylthio group, a substituted or unsubstituted heterocyclic group, or a substituted or unsubstituted amine group, or is bonded to an adjacent substituent to form a substituted or unsubstituted ring; and

r11 is an integer from 0 to 9, and when r11 is 2 or higher, the R11s are the same as or different from each other.

In an exemplary embodiment of the present specification, Ar1 and Ar2 are the same as or different from each other, and are each independently one selected from the group consisting of a substituted or unsubstituted aromatic hydrocarbon ring, a substituted or unsubstituted aliphatic hydrocarbon ring, and a substituted or unsubstituted aromatic hetero ring, or a ring in which two or more rings selected from the group are fused.

In an exemplary embodiment of the present specification, Ar1 and Ar2 are the same as or different from each other, and are each independently a substituted or unsubstituted aryl group or a substituted or unsubstituted heterocyclic group.

In an exemplary embodiment of the present specification, Ar1 and Ar2 are the same as or different from each other, and are each independently a substituted or unsubstituted C6-C30 aryl group or a substituted or unsubstituted C2-C30 heterocyclic group.

In an exemplary embodiment of the present specification, Ar1 and Ar2 are the same as or different from each other, and are each independently a substituted or unsubstituted C6-C20 aryl group or a substituted or unsubstituted C2-C20 heterocyclic group.

In an exemplary embodiment of the present specification, Ar1 and Ar2 are the same as or different from each other, and are each independently a substituted or unsubstituted aryl group, a fused ring group of a substituted or unsubstituted aromatic hydrocarbon ring and an aliphatic hydrocarbon ring, or a substituted or unsubstituted heterocyclic group.

In an exemplary embodiment of the present specification, Ar1 and Ar2 are the same as or different from each other, and are each independently a substituted or unsubstituted C6-C20 aryl group, a C9-C20 fused ring group of a substituted or unsubstituted aromatic hydrocarbon ring and an aliphatic hydrocarbon ring, or a substituted or unsubstituted C2-C20 heterocyclic group.

In an exemplary embodiment of the present specification, Ar1 and Ar2 are the same as or different from each other, and are each independently a C6-C20 aryl group; a C9-C20 fused ring group of a substituted or unsubstituted aromatic hydrocarbon ring and an aliphatic hydrocarbon ring, which is unsubstituted or substituted with a C1-C6 alkyl group; or a C2-C20 heterocyclic group.

In an exemplary embodiment of the present specification, Ar1 and Ar2 are the same as or different from each other, and are each independently a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted phenanthrenyl group, a substituted or unsubstituted triphenylenyl group, a substituted or unsubstituted fluorenyl group, a substituted or unsubstituted dihydroindene group, a substituted or unsubstituted tetrahydronaphthalene group, a substituted or unsubstituted dibenzofuran group, a substituted or unsubstituted dibenzothiophene group, a substituted or unsubstituted naphthobenzofuran group, or a substituted or unsubstituted naphthobenzothiophene group.

In an exemplary embodiment of the present specification, Ar1 and Ar2 are the same as or different from each other, and are each independently a phenyl group; a biphenyl group; a terphenyl group; a quaterphenyl group; a naphthyl group; a phenanthrenyl group; a triphenylenyl group; a fluorenyl group which is unsubstituted or substituted with a methyl group or a phenyl group; a dihydroindene group which is unsubstituted or substituted with a methyl group; a tetrahydronaphthalene group which is unsubstituted or substituted with a methyl group; a dibenzofuran group; a dibenzothiophene group; a naphthobenzofuran group; or a naphthobenzothiophene group.

In an exemplary embodiment of the present specification, Ar1 is a substituted or unsubstituted polycyclic C10-C30 aryl group.

In an exemplary embodiment of the present specification, Ar1 is a substituted or unsubstituted polycyclic C10-C20 aryl group.

In an exemplary embodiment of the present specification, Ar1 is a substituted or unsubstituted naphthyl group; or a substituted or unsubstituted phenanthrenyl group.

In an exemplary embodiment of the present specification, Ar1 is a substituted or unsubstituted 1-naphthyl group, a substituted or unsubstituted 2-naphthyl group, or a substituted or unsubstituted 9-phenanthrenyl group.

In an exemplary embodiment of the present specification, Ar2 is a substituted or unsubstituted C6-C20 aryl group, a C9-C20 fused ring group of a substituted or unsubstituted aromatic hydrocarbon ring and an aliphatic hydrocarbon ring, or a substituted or unsubstituted C2-C20 heterocyclic group.

In an exemplary embodiment of the present specification, Ar2 is a C6-C20 aryl group; a C9-C20 fused ring group of a substituted or unsubstituted aromatic hydrocarbon ring and an aliphatic hydrocarbon ring, which is unsubstituted or substituted with a C1-C6 alkyl group; or a C2-C20 heterocyclic group.

In an exemplary embodiment of the present specification, Ar2 is a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted phenanthrenyl group, a substituted or unsubstituted triphenylenyl group, a substituted or unsubstituted fluorenyl group, a substituted or unsubstituted dihydroindene group, a substituted or unsubstituted tetrahydronaphthalene group, a substituted or unsubstituted dibenzofuran group, a substituted or unsubstituted dibenzothiophene group, a substituted or unsubstituted naphthobenzofuran group, or a substituted or unsubstituted naphthobenzothiophene group.

In an exemplary embodiment of the present specification, Ar2 is a phenyl group; a biphenyl group; a terphenyl group; a quaterphenyl group; a naphthyl group; a phenanthrenyl group; a triphenylenyl group; a fluorenyl group which is unsubstituted or substituted with a methyl group or a phenyl group; a dihydroindene group which is unsubstituted or substituted with a methyl group; a tetrahydronaphthalene group which is unsubstituted or substituted with a methyl group; a dibenzofuran group; a dibenzothiophene group; a naphthobenzofuran group; or a naphthobenzothiophene group.

In an exemplary embodiment of the present specification, Ar1 and Ar2 are each independently selected from the following structures:

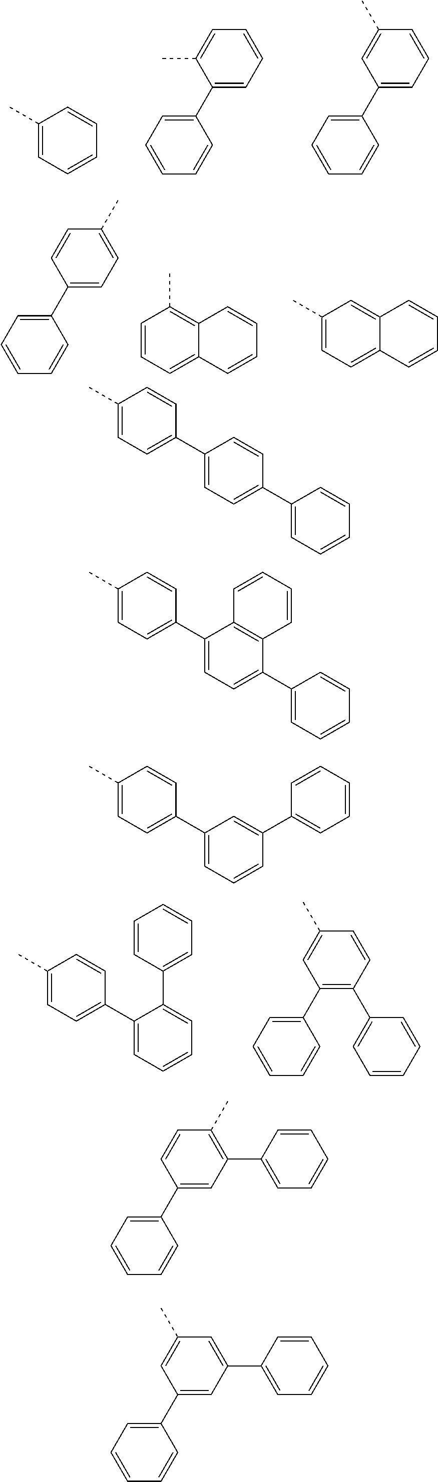

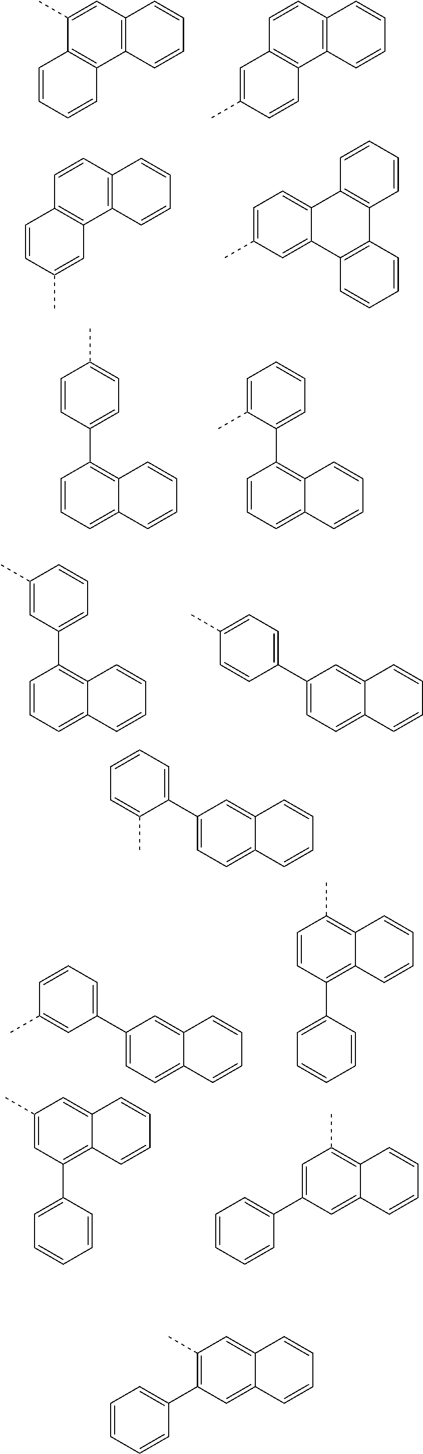

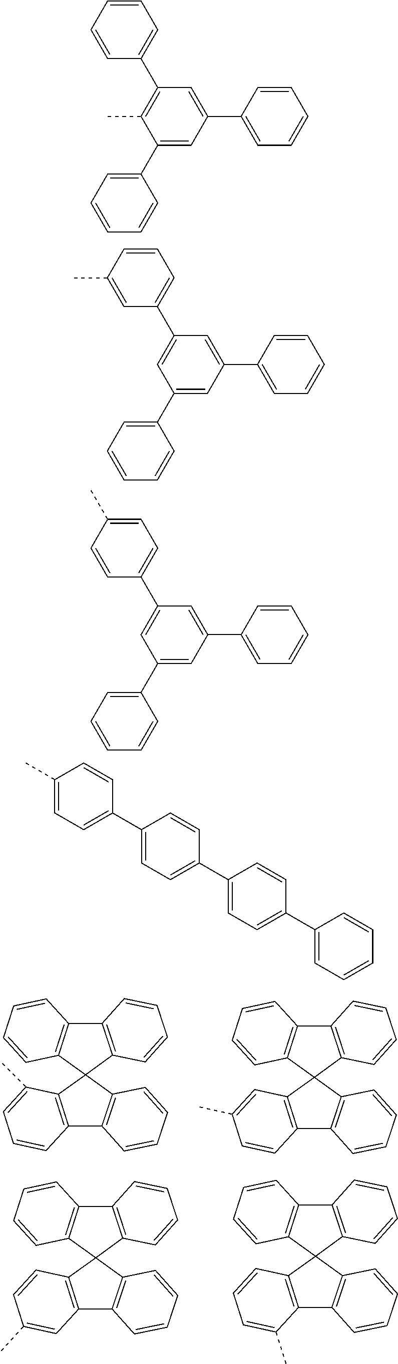

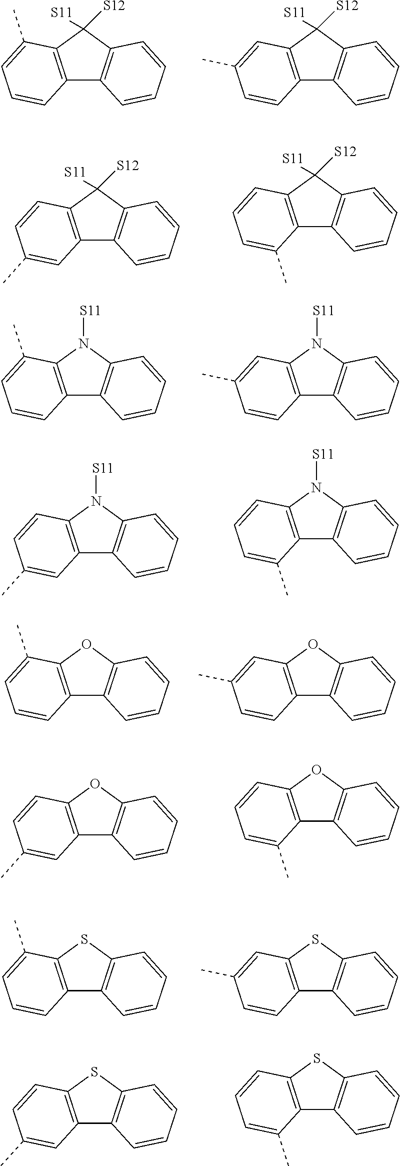

wherein in the structures:

a dotted line denotes a bonding position; and

S11 an S12 are the same as or different from each other other, and are each independently hydrogen, a substituted or unsubstituted alkyl group, or a substituted or unsubstituted aryl group.

In an exemplary embodiment of the present specification, L1 to L3 are the same as or different from each other, and are each independently a direct bond, a substituted or unsubstituted arylene group, or a substituted or unsubstituted divalent heterocyclic group.

In an exemplary embodiment of the present specification, L1 to L3 are the same as or different from each other, and are each independently a direct bond, a substituted or unsubstituted C6-C60 arylene group, or a substituted or unsubstituted C2-C60 divalent heterocyclic group.

In an exemplary embodiment of the present specification, L1 to L3 are the same as or different from each other, and are each independently a direct bond; a substituted or unsubstituted C6-C30 arylene group; or a substituted or unsubstituted C2-C60 divalent heterocyclic group.

In an exemplary embodiment of the present specification, L1 to L3 are the same as or different from each other, and are each independently a direct bond or a substituted or unsubstituted C6-C20 arylene group.

In an exemplary embodiment of the present specification, L1 to L3 are the same as or different from each other, and are each independently a direct bond, a substituted or unsubstituted phenylene group, a substituted or unsubstituted biphenylene group, a substituted or unsubstituted terphenylene group, or a substituted or unsubstituted naphthylene group.

In an exemplary embodiment of the present specification, the case where all of L1 to L3 are a direct bond is excluded.

In an exemplary embodiment of the present specification, one or more of L1 to L3 are a substituted or unsubstituted arylene group.

In an exemplary embodiment of the present specification, one or more of L1 to L3 are a substituted or unsubstituted C6-C30 arylene group.

In an exemplary embodiment of the present specification, L1 to L3 are the same as or different from each other, and are each independently a direct bond or one selected from among the following structures:

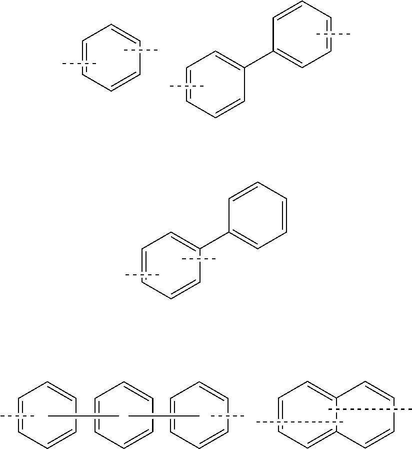

wherein in the structures, the dotted line is a position which is linked to Chemical Formula 2.

In an exemplary embodiment of the present specification, one or more, two or more, or all of L1 to L3 are selected from the structures.

In an exemplary embodiment of the present specification, L1 to L3 are the same as or different from each other, and are each independently a direct bond or one selected from among the following structures:

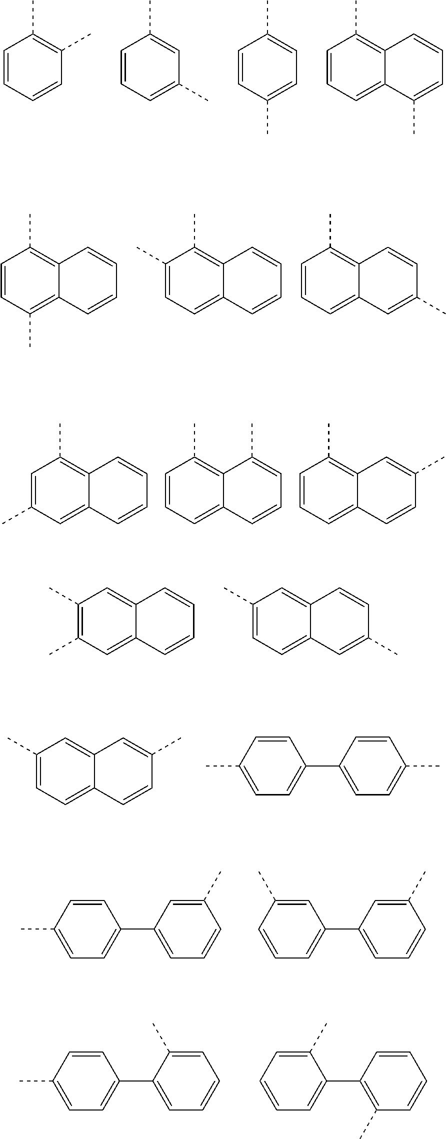

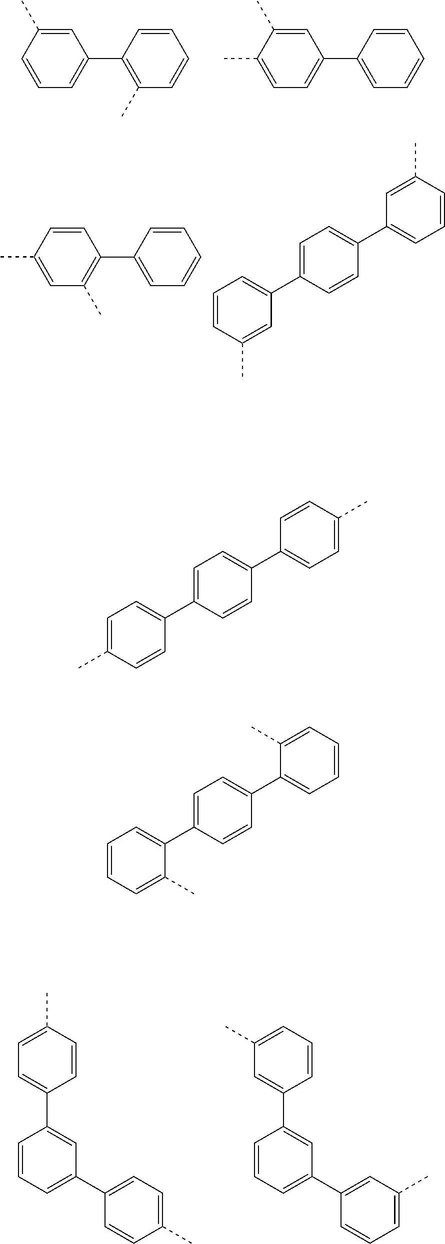

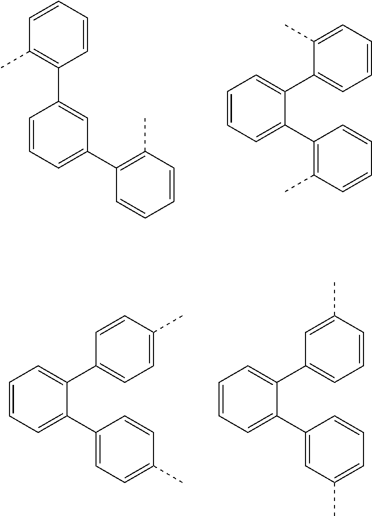

wherein in the structures, the dotted line is a position which is linked to Chemical Formula 2.

In an exemplary embodiment of the present specification, one or more, two or more, or all of L1 to L3 are selected from the structures.

In an exemplary embodiment of the present specification, R11 is hydrogen, deuterium, a cyano group, a halogen group, a substituted or unsubstituted C1-C10 alkyl group, a substituted or unsubstituted C1-C10 alkoxy group, a substituted or unsubstituted C1-C10 alkylthio group, a substituted or unsubstituted C3-C30 cycloalkyl group, a substituted or unsubstituted silyl group, a substituted or unsubstituted C6-C30 aryl group, a substituted or unsubstituted C6-C30 aryloxy group, a substituted or unsubstituted C6-C30 arylthio group, a substituted or unsubstituted C2-C30 heterocyclic group, or a substituted or unsubstituted amine group, or is bonded to an adjacent substituent to form a substituted or unsubstituted C3-C30 ring.

In an exemplary embodiment of the present specification, R11 is hydrogen or deuterium.

In an exemplary embodiment of the present specification, R11 is hydrogen.

In an exemplary embodiment of the present specification, Chemical Formula 2 is the following Chemical Formula 201:

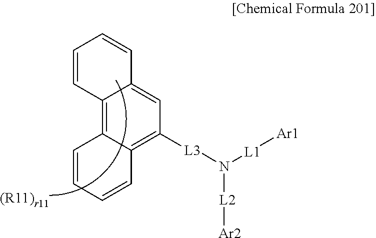

wherein in Chemical Formula 201:

L1 to L3, Ar1, Ar2, R11, and r11 are those defined in Chemical Formula 2.

In an exemplary embodiment of the present specification, Chemical Formula 2 is one compound selected from among the following compounds:

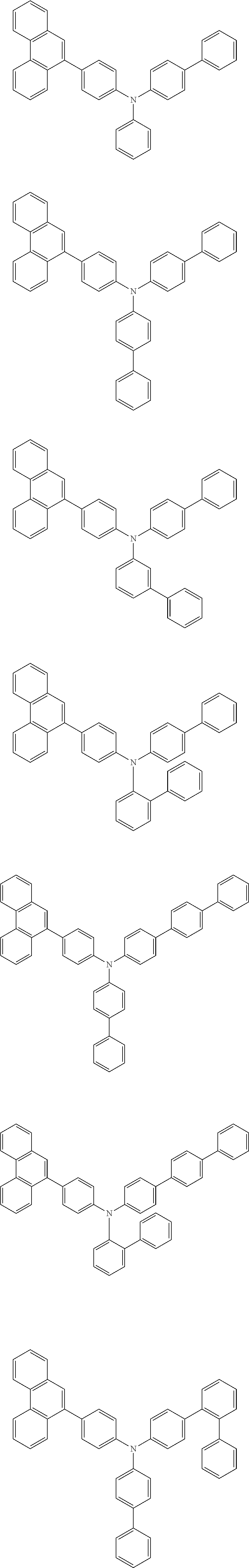

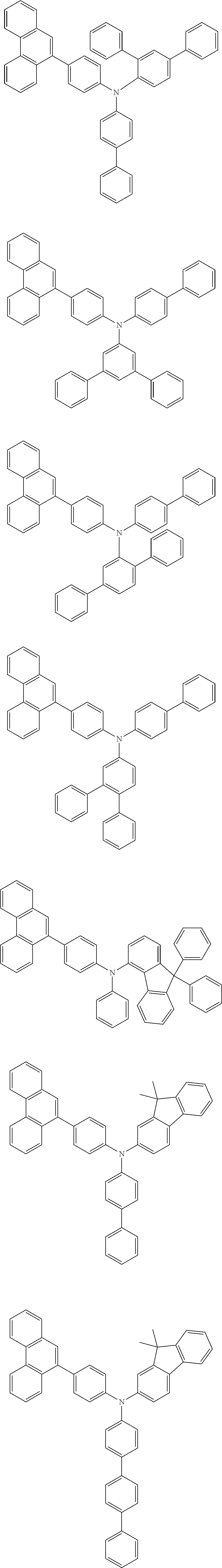

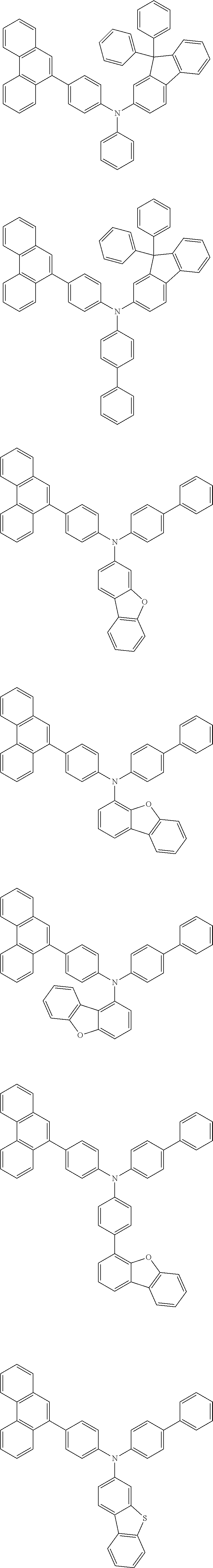

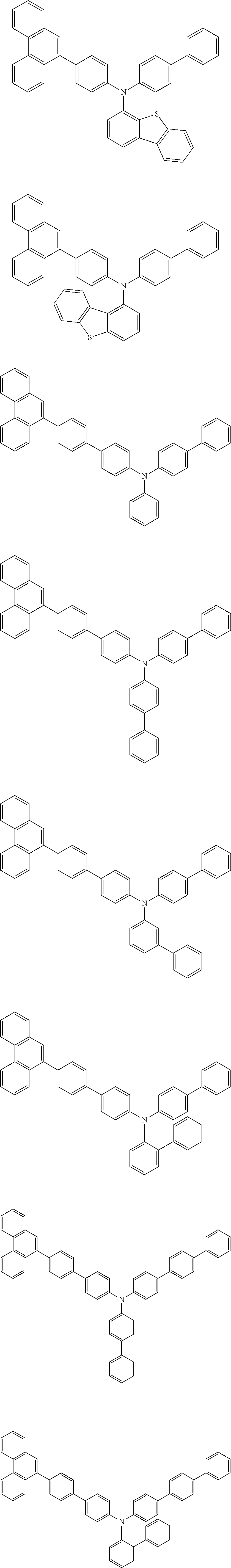

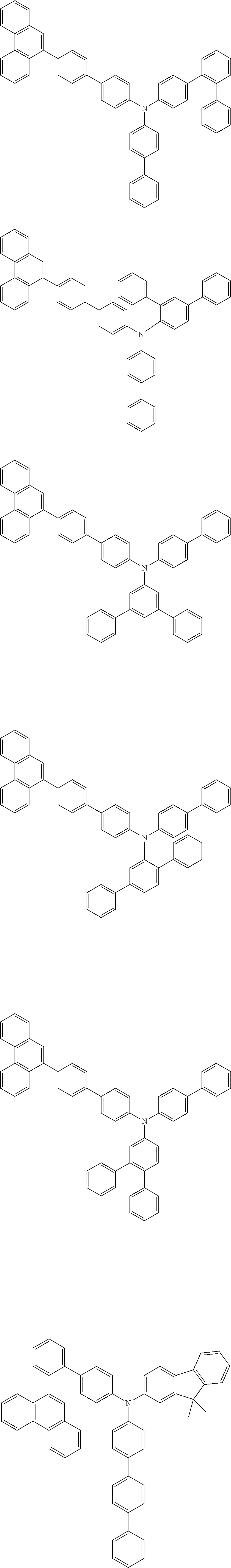

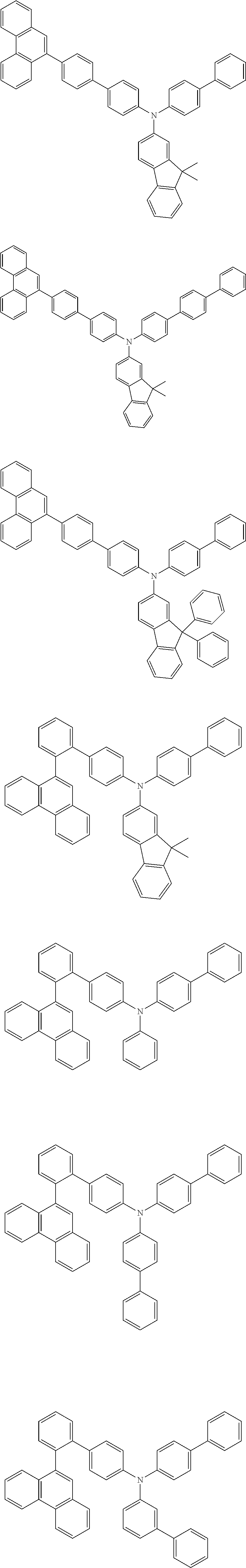

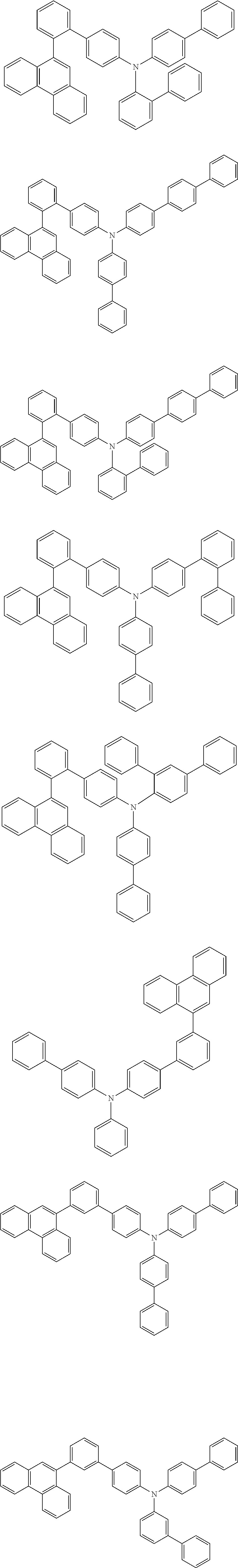

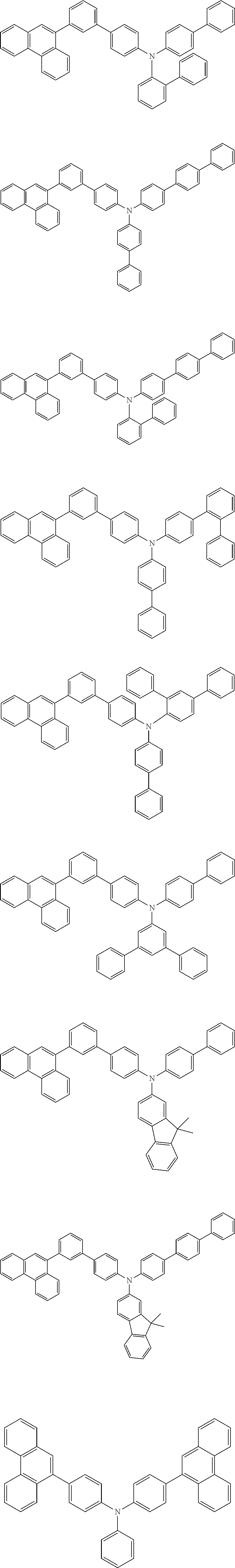

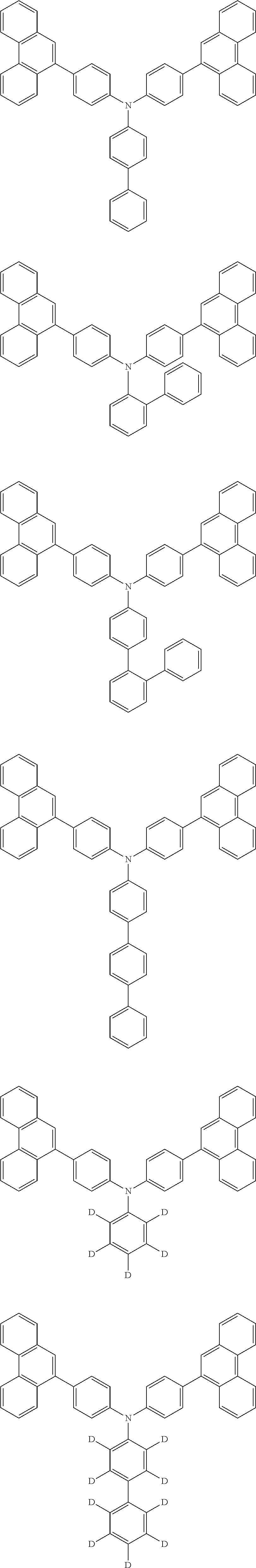

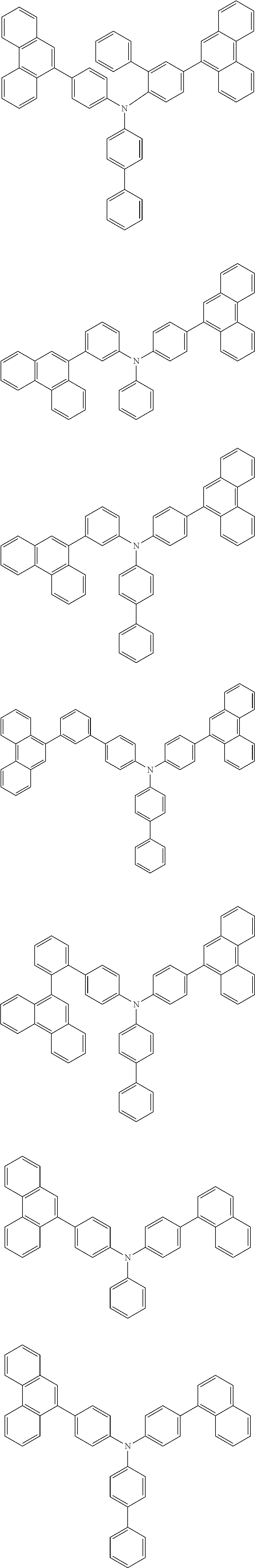

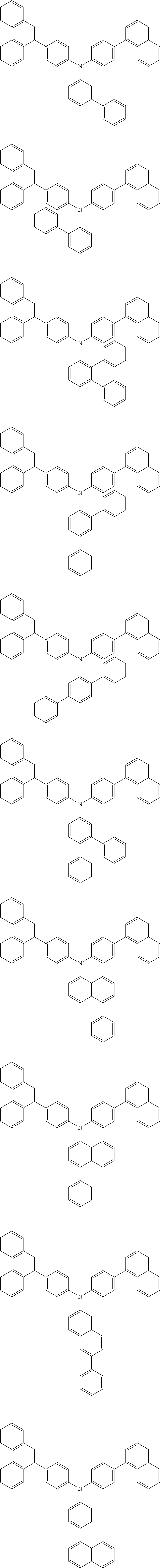

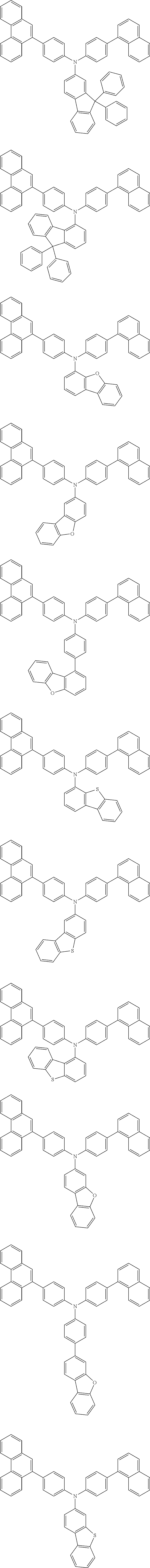

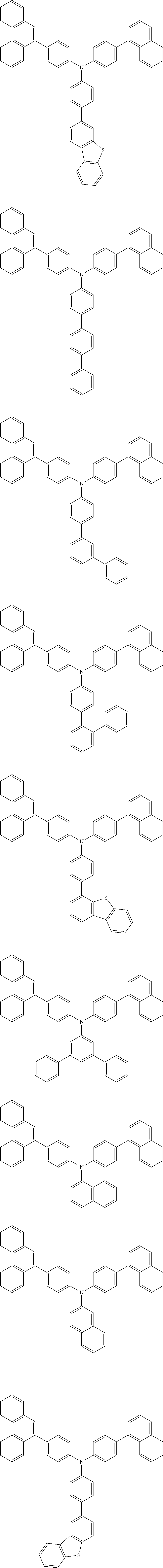

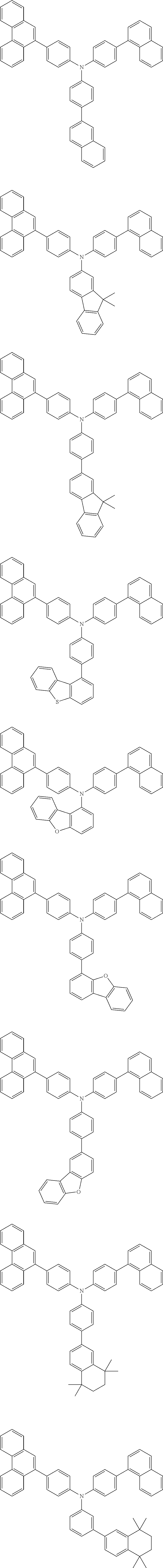

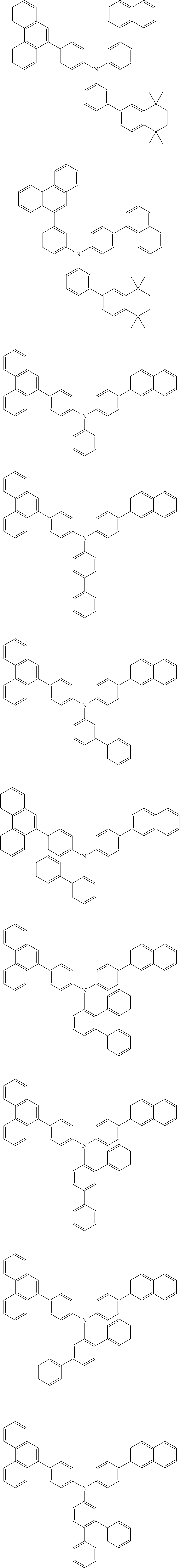

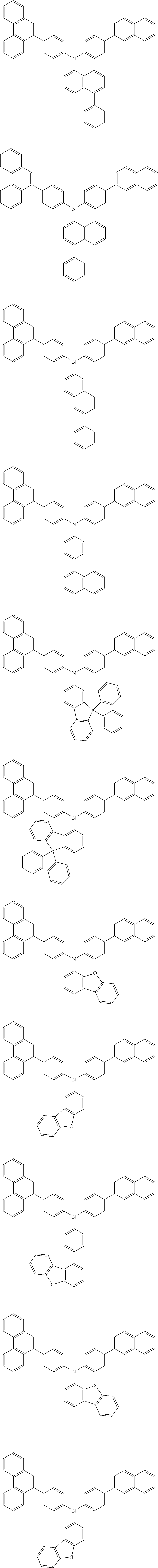

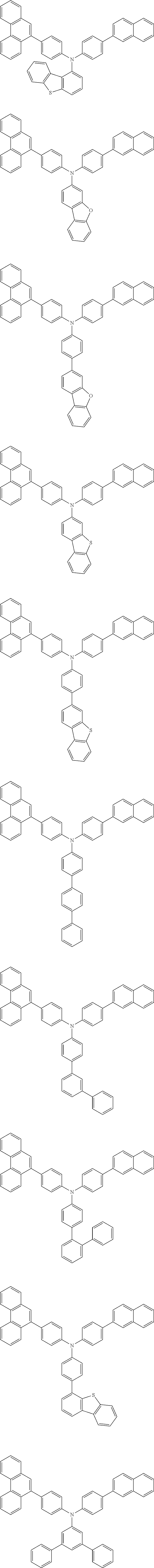

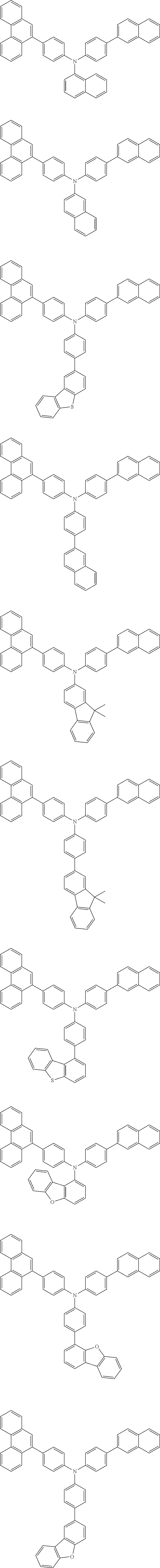

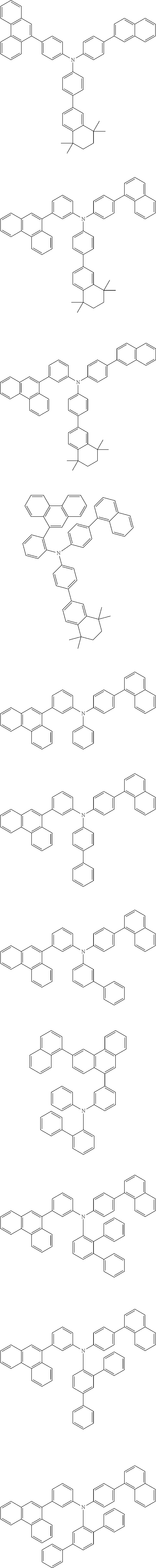

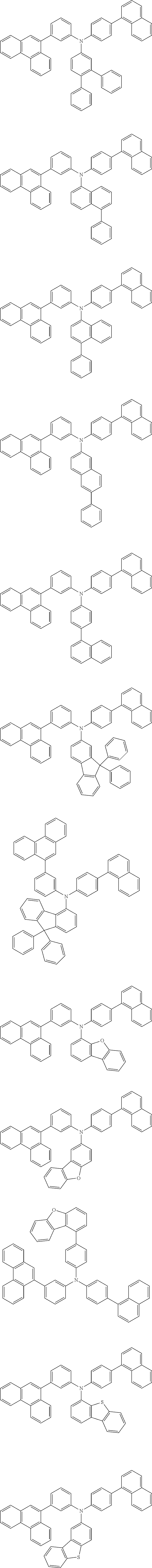

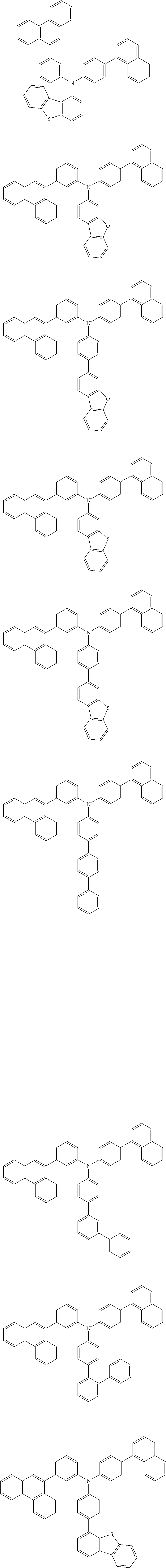

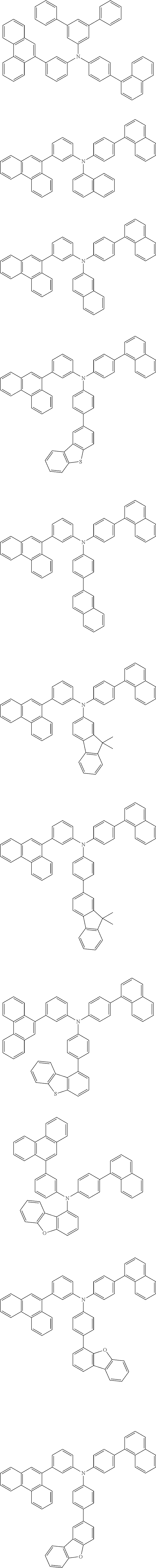

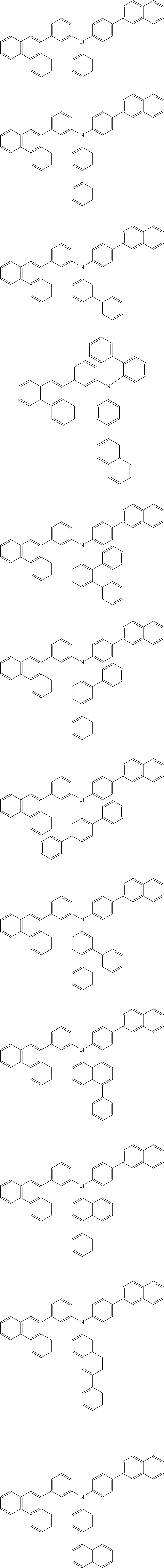

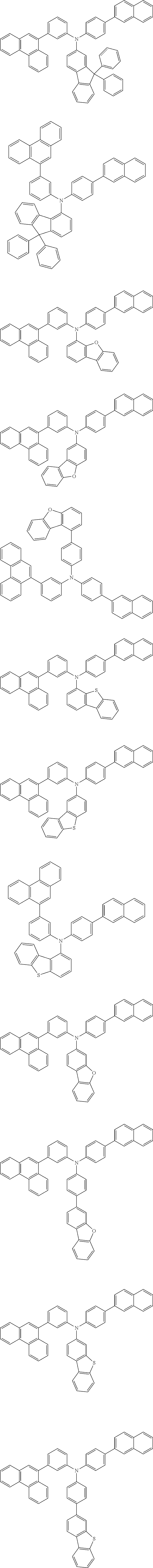

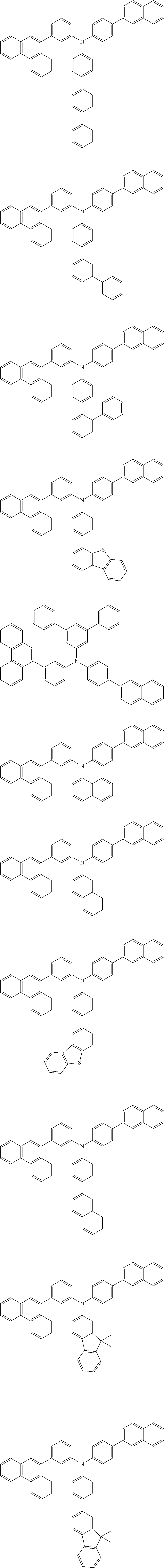

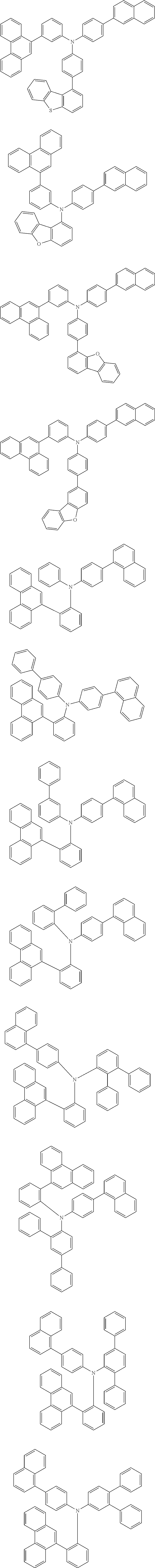

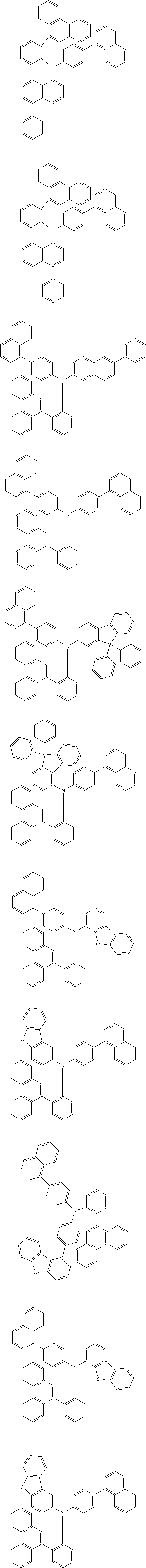

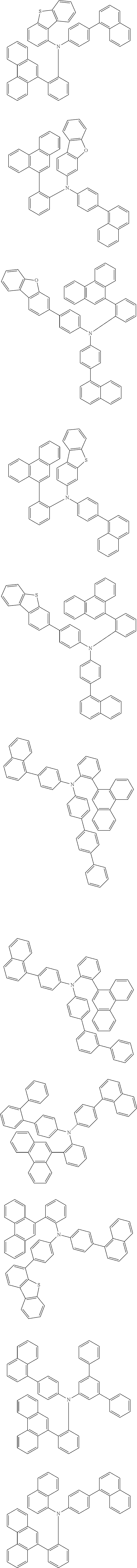

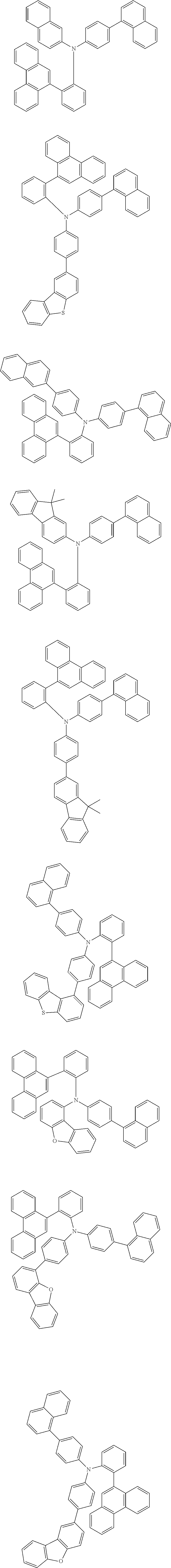

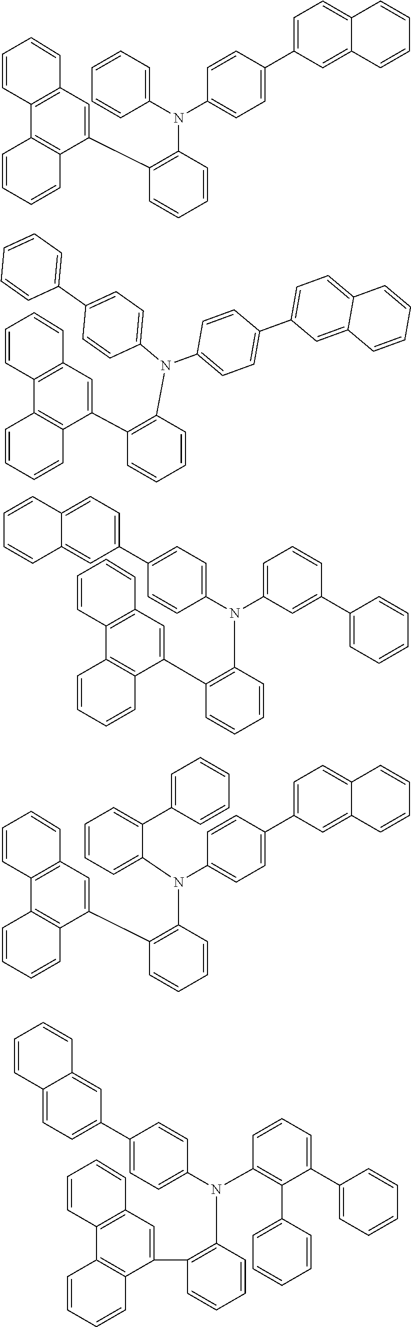

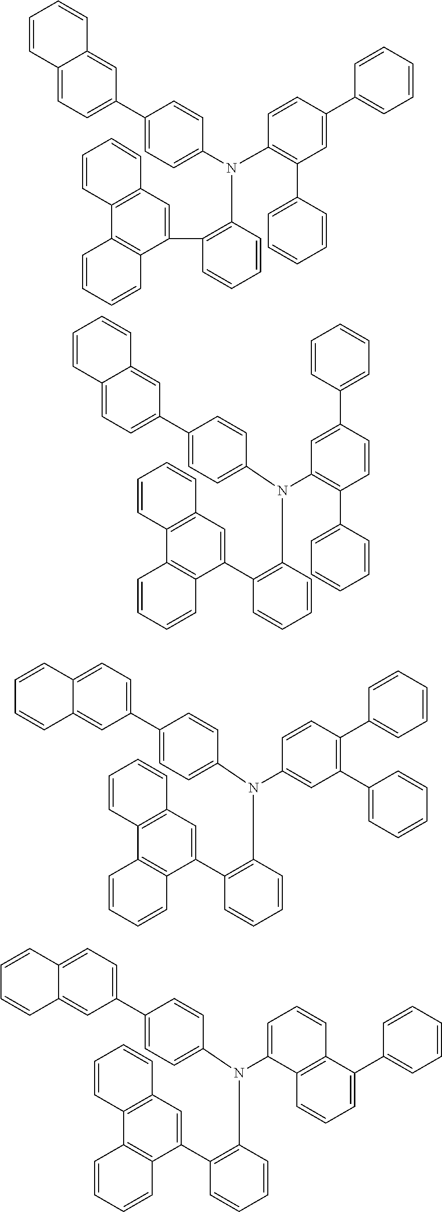

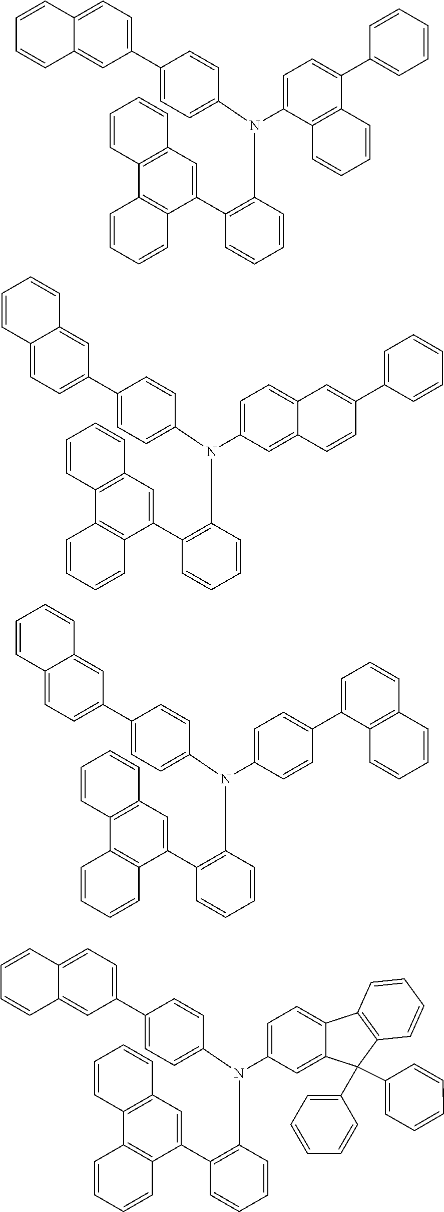

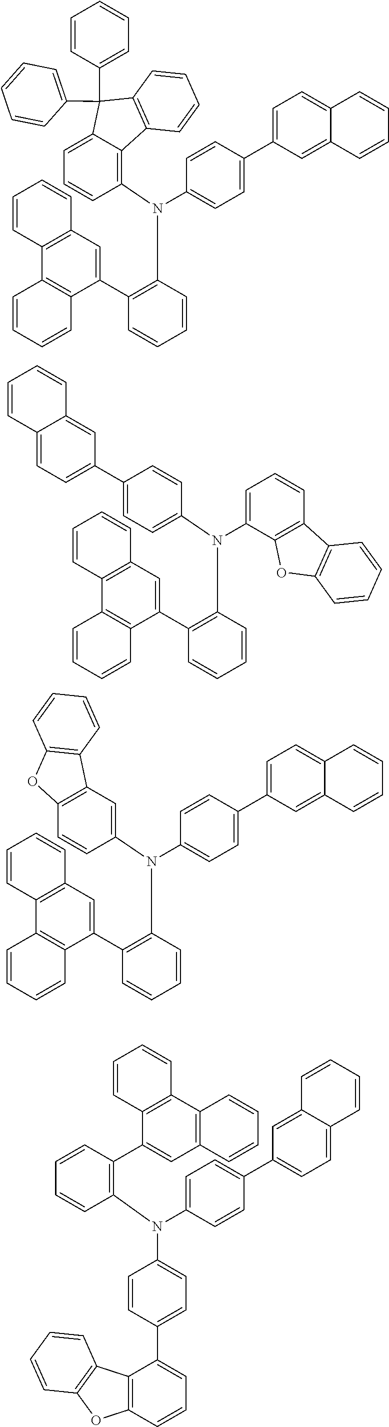

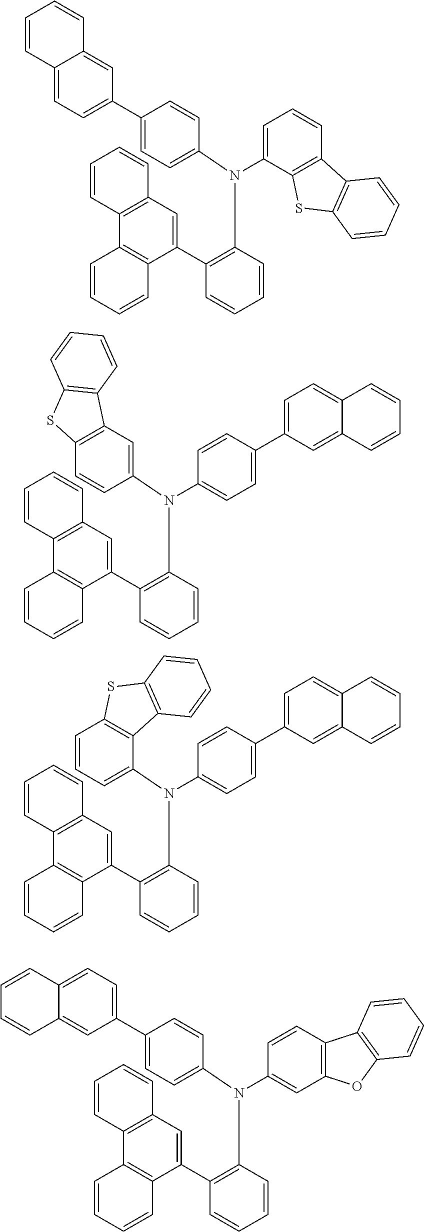

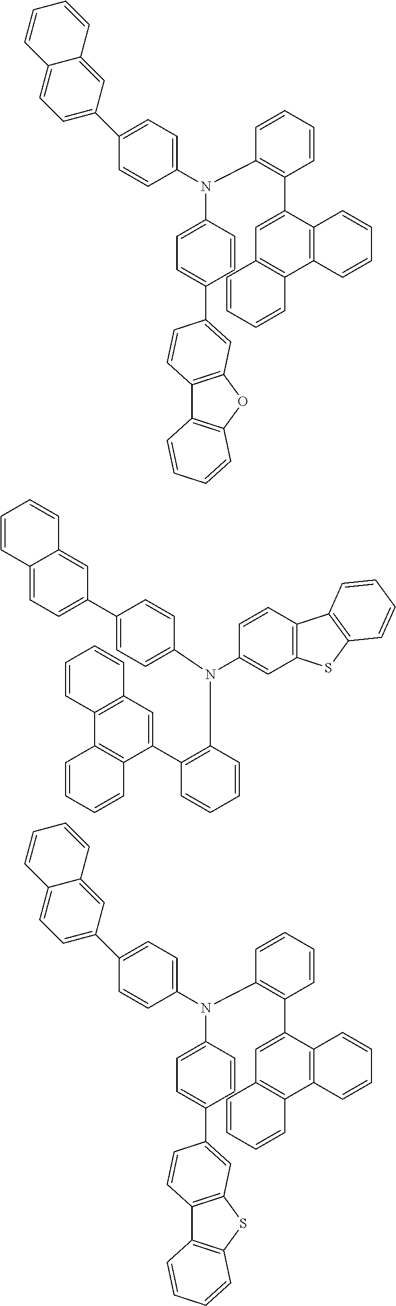

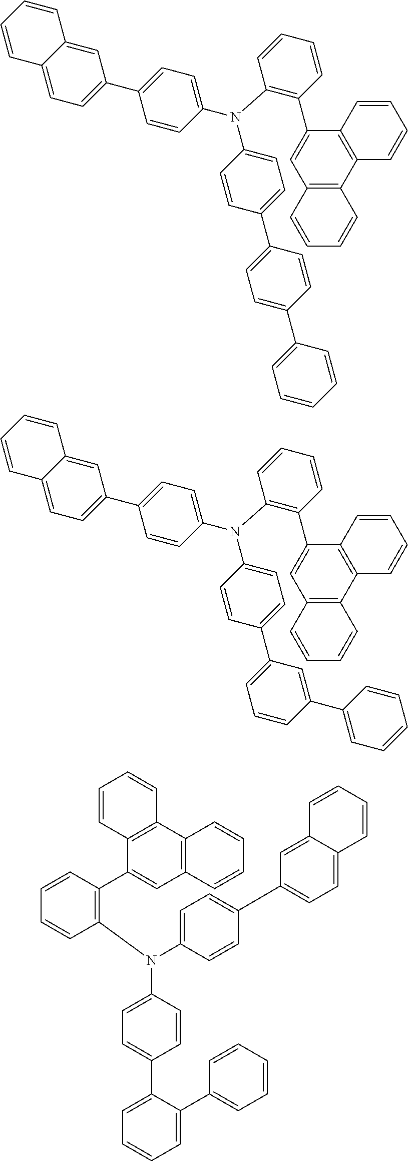

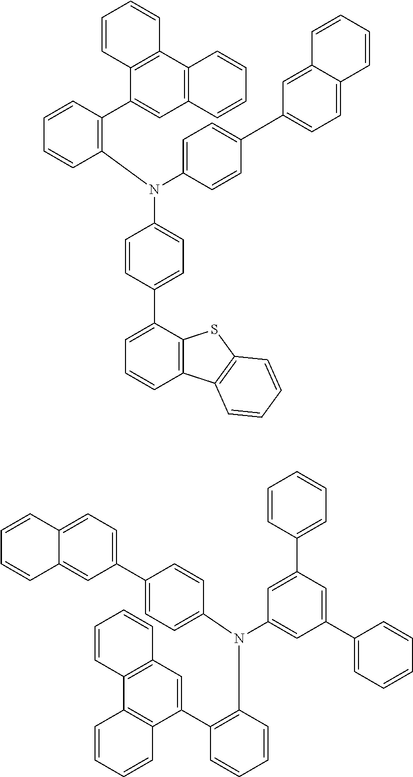 [SMILES: c1ccc(N(c2ccc(-c3ccc4ccccc4c3)cc2)c2ccc(-c3cccc4c3sc3ccccc34)cc2)c(-c2cc3ccccc3c3ccccc23)c1 ; c1ccc(-c2cc(-c3ccccc3)cc(N(c3ccc(-c4ccc5ccccc5c4)cc3)c3ccccc3-c3cc4ccccc4c4ccccc34)c2)cc1]

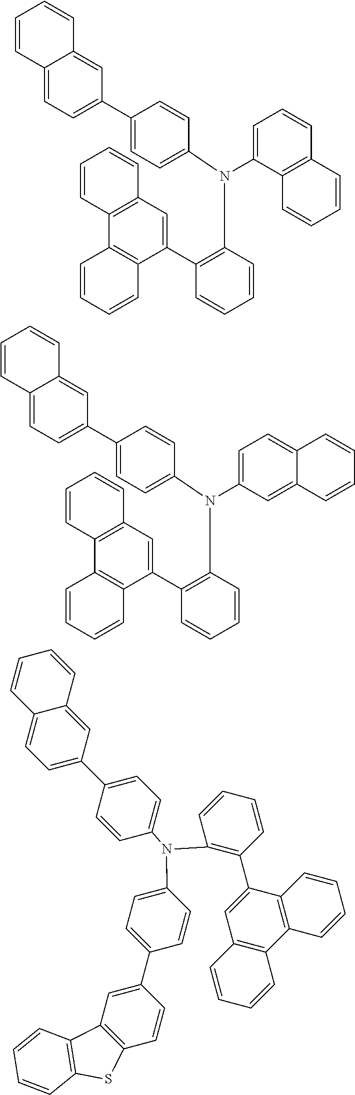

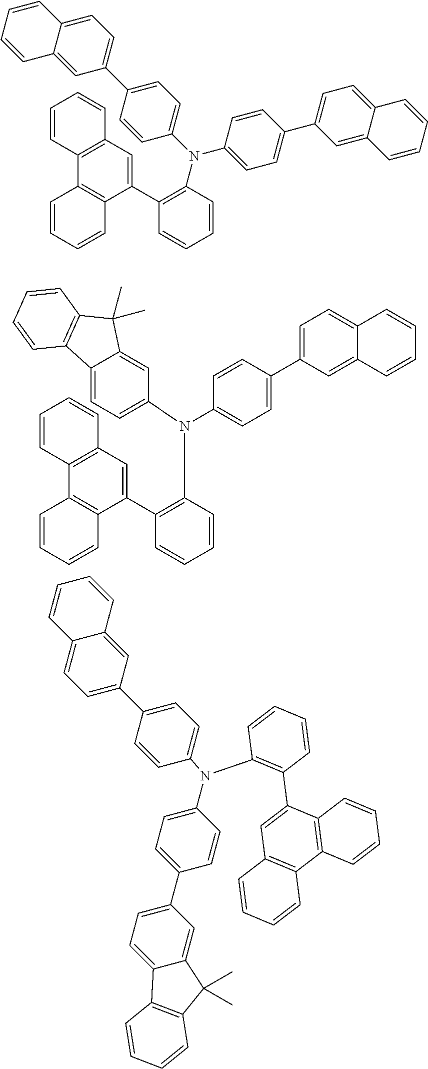

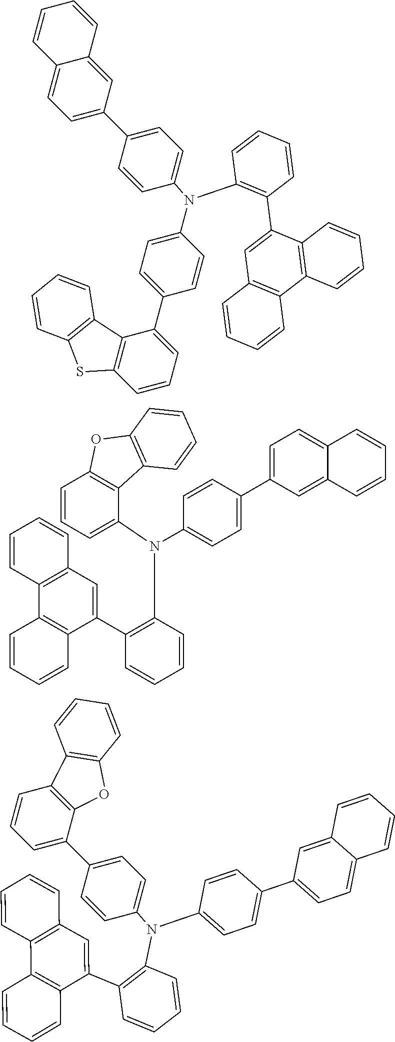

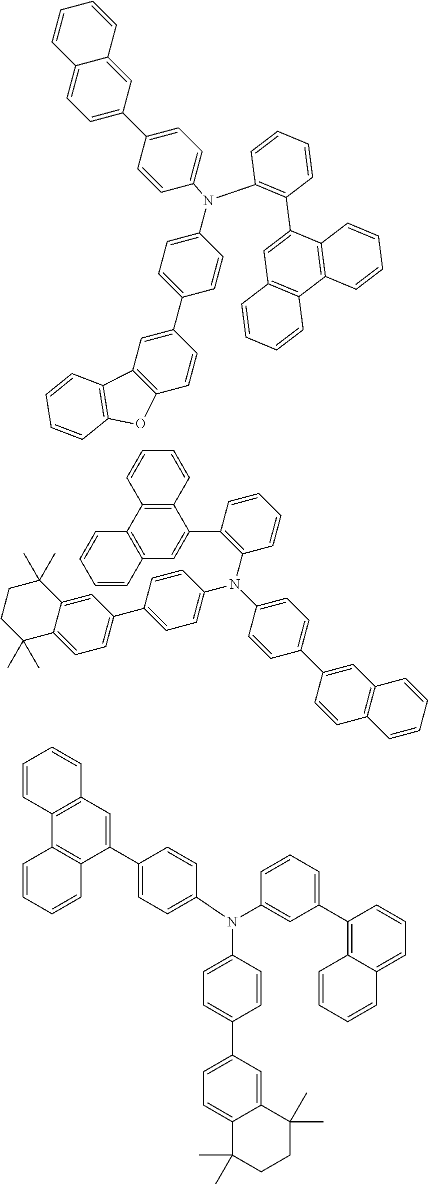

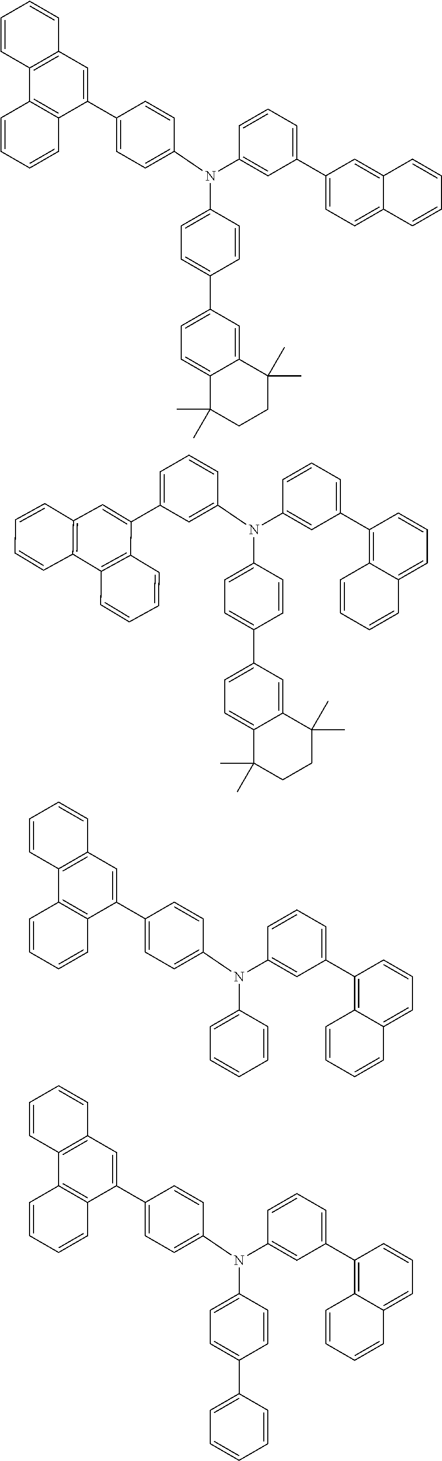

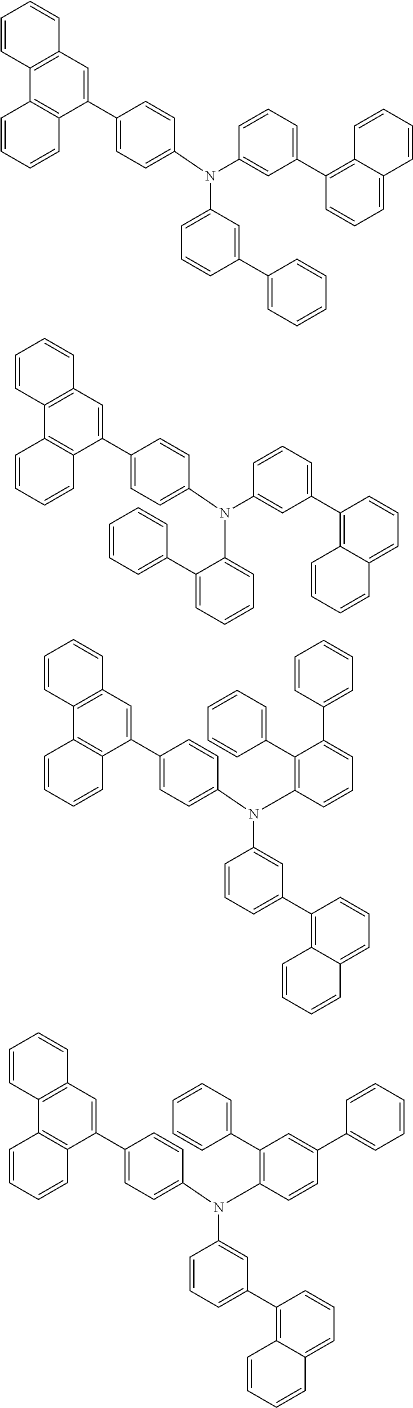

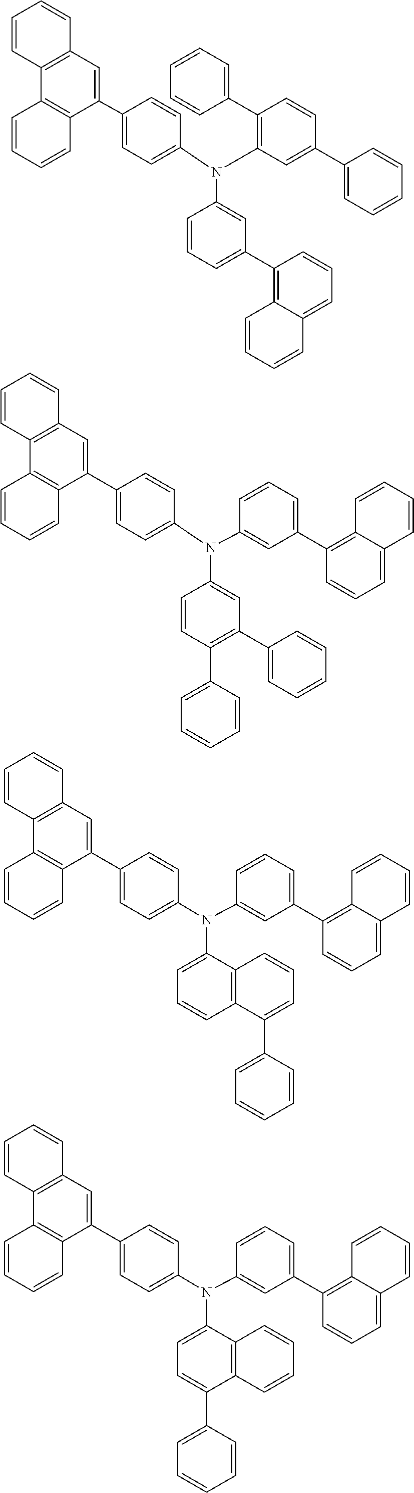 [SMILES: c1ccc(-c2ccc(-c3ccccc3)c(N(c3ccc(-c4cc5ccccc5c5ccccc45)cc3)c3cccc(-c4cccc5ccccc45)c3)c2)cc1 ; c1ccc(-c2ccc(N(c3ccc(-c4cc5ccccc5c5ccccc45)cc3)c3cccc(-c4cccc5ccccc45)c3)cc2-c2ccccc2)cc1 ; c1ccc(-c2cccc3c(N(c4ccc(-c5cc6ccccc6c6ccccc56)cc4)c4cccc(-c5cccc6ccccc56)c4)cccc23)cc1 ; c1ccc(-c2ccc(N(c3ccc(-c4cc5ccccc5c5ccccc45)cc3)c3cccc(-c4cccc5ccccc45)c3)c3ccccc23)cc1]

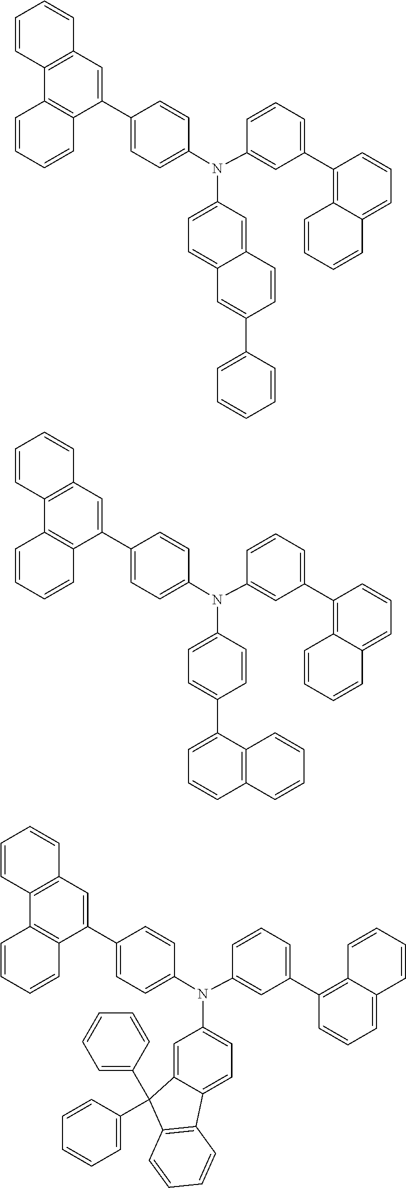 [SMILES: c1ccc(-c2ccc3cc(N(c4ccc(-c5cc6ccccc6c6ccccc56)cc4)c4cccc(-c5cccc6ccccc56)c4)ccc3c2)cc1 ; c1cc(-c2cccc3ccccc23)cc(N(c2ccc(-c3cccc4ccccc34)cc2)c2ccc(-c3cc4ccccc4c4ccccc34)cc2)c1 ; c1ccc(C2(c3ccccc3)c3ccccc3-c3ccc(N(c4ccc(-c5cc6ccccc6c6ccccc56)cc4)c4cccc(-c5cccc6ccccc56)c4)cc32)cc1]

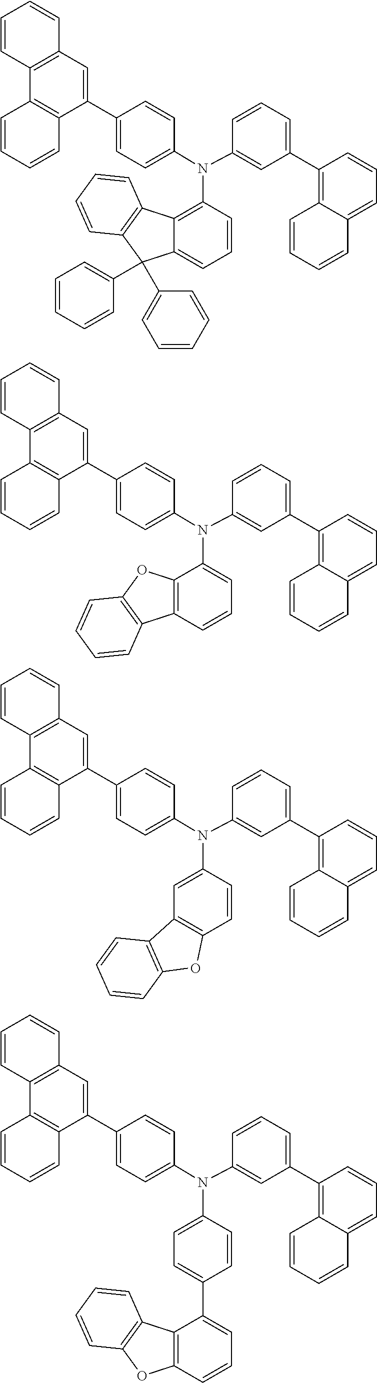

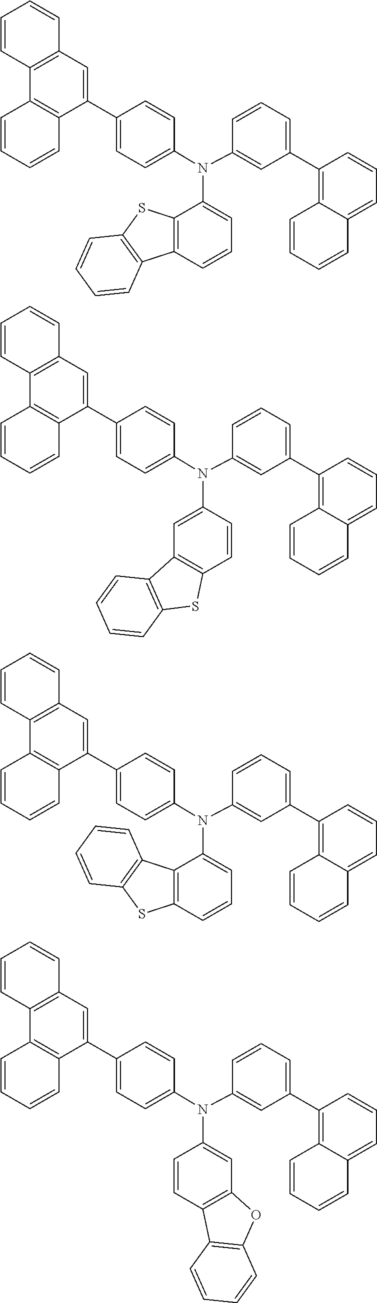

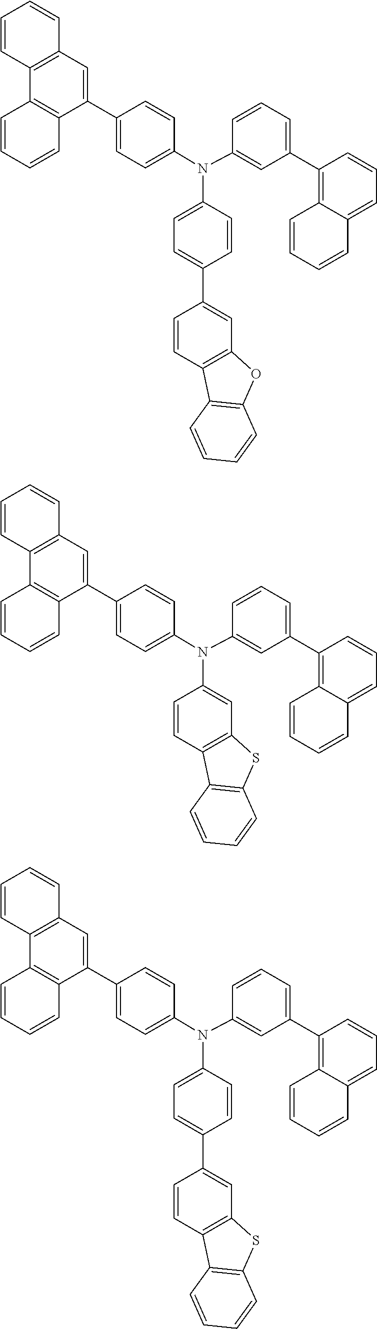 [SMILES: c1cc(-c2cccc3ccccc23)cc(N(c2ccc(-c3ccc4c(c3)oc3ccccc34)cc2)c2ccc(-c3cc4ccccc4c4ccccc34)cc2)c1 ; c1cc(-c2cccc3ccccc23)cc(N(c2ccc(-c3cc4ccccc4c4ccccc34)cc2)c2ccc3c(c2)sc2ccccc23)c1 ; c1cc(-c2cccc3ccccc23)cc(N(c2ccc(-c3ccc4c(c3)sc3ccccc34)cc2)c2ccc(-c3cc4ccccc4c4ccccc34)cc2)c1]

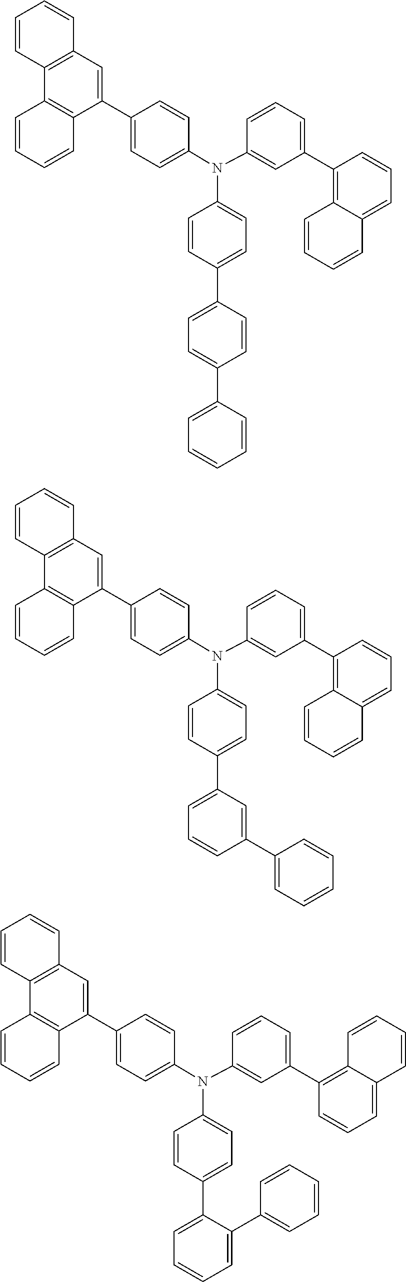 [SMILES: c1ccc(-c2ccc(-c3ccc(N(c4ccc(-c5cc6ccccc6c6ccccc56)cc4)c4cccc(-c5cccc6ccccc56)c4)cc3)cc2)cc1 ; c1ccc(-c2cccc(-c3ccc(N(c4ccc(-c5cc6ccccc6c6ccccc56)cc4)c4cccc(-c5cccc6ccccc56)c4)cc3)c2)cc1 ; c1ccc(-c2ccccc2-c2ccc(N(c3ccc(-c4cc5ccccc5c5ccccc45)cc3)c3cccc(-c4cccc5ccccc45)c3)cc2)cc1]

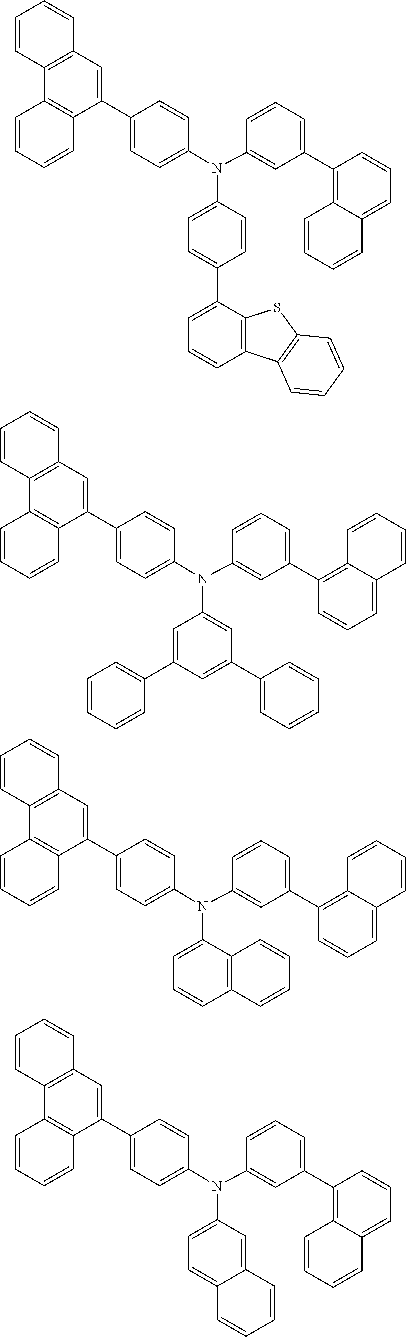 [SMILES: c1cc(-c2cccc3ccccc23)cc(N(c2ccc(-c3cc4ccccc4c4ccccc34)cc2)c2ccc(-c3cccc4c3sc3ccccc34)cc2)c1 ; c1ccc(-c2cc(-c3ccccc3)cc(N(c3ccc(-c4cc5ccccc5c5ccccc45)cc3)c3cccc(-c4cccc5ccccc45)c3)c2)cc1 ; c1cc(-c2cccc3ccccc23)cc(N(c2ccc(-c3cc4ccccc4c4ccccc34)cc2)c2cccc3ccccc23)c1 ; c1cc(-c2cccc3ccccc23)cc(N(c2ccc(-c3cc4ccccc4c4ccccc34)cc2)c2ccc3ccccc3c2)c1]

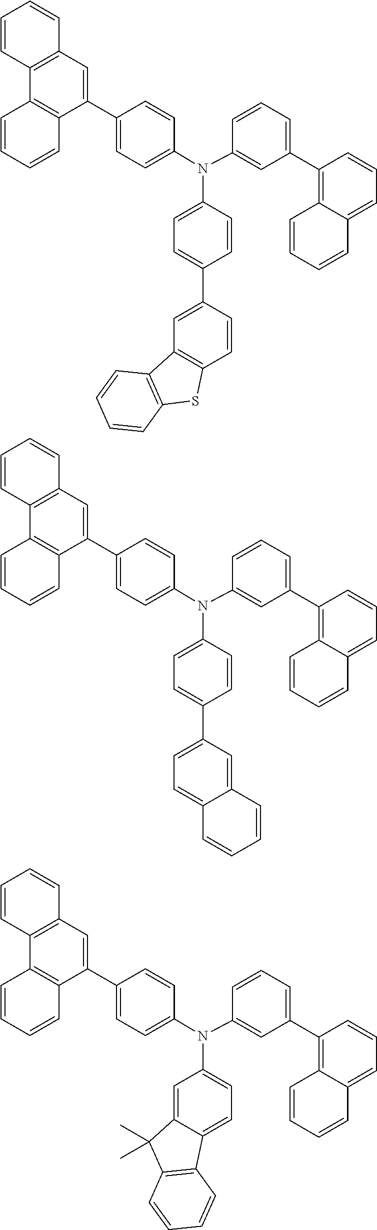 [SMILES: c1cc(-c2cccc3ccccc23)cc(N(c2ccc(-c3ccc4sc5ccccc5c4c3)cc2)c2ccc(-c3cc4ccccc4c4ccccc34)cc2)c1 ; c1cc(-c2cccc3ccccc23)cc(N(c2ccc(-c3ccc4ccccc4c3)cc2)c2ccc(-c3cc4ccccc4c4ccccc34)cc2)c1 ; CC1(C)c2ccccc2-c2ccc(N(c3ccc(-c4cc5ccccc5c5ccccc45)cc3)c3cccc(-c4cccc5ccccc45)c3)cc21]

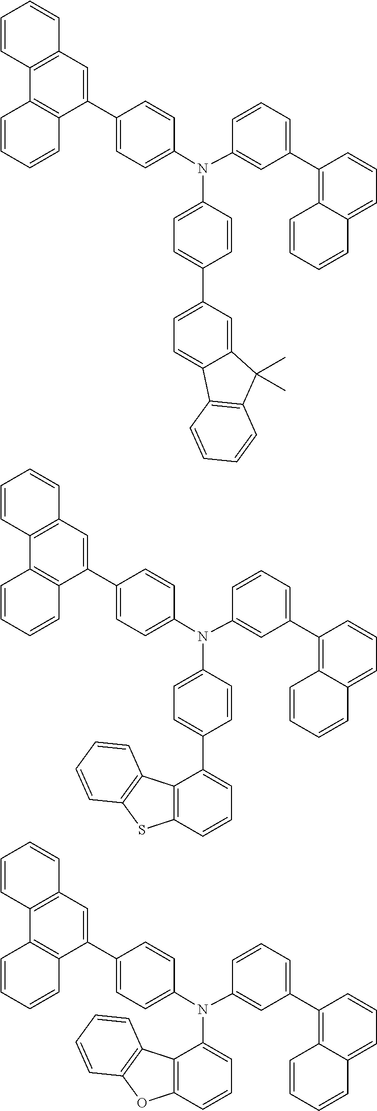 [SMILES: CC1(C)c2ccccc2-c2ccc(-c3ccc(N(c4ccc(-c5cc6ccccc6c6ccccc56)cc4)c4cccc(-c5cccc6ccccc56)c4)cc3)cc21 ; c1cc(-c2cccc3ccccc23)cc(N(c2ccc(-c3cc4ccccc4c4ccccc34)cc2)c2ccc(-c3cccc4sc5ccccc5c34)cc2)c1 ; c1cc(-c2cccc3ccccc23)cc(N(c2ccc(-c3cc4ccccc4c4ccccc34)cc2)c2cccc3oc4ccccc4c23)c1]

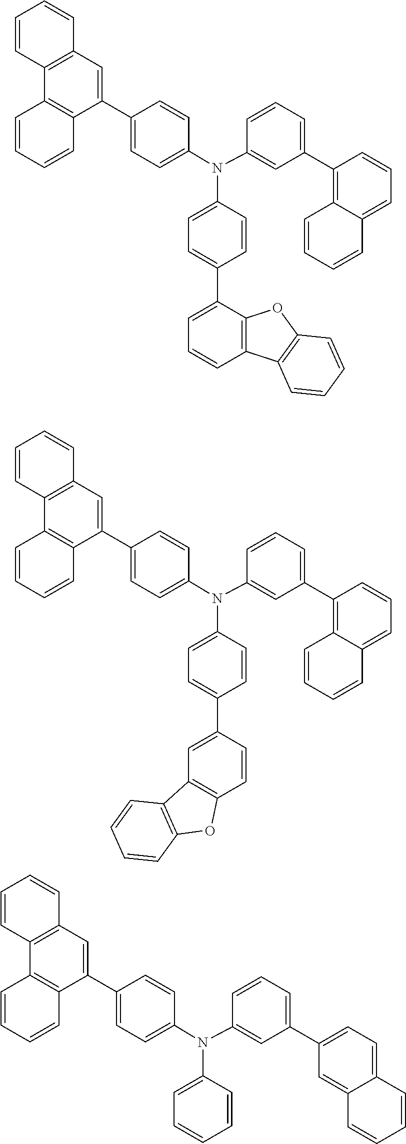 [SMILES: c1cc(-c2cccc3ccccc23)cc(N(c2ccc(-c3cc4ccccc4c4ccccc34)cc2)c2ccc(-c3cccc4c3oc3ccccc34)cc2)c1 ; c1cc(-c2cccc3ccccc23)cc(N(c2ccc(-c3ccc4oc5ccccc5c4c3)cc2)c2ccc(-c3cc4ccccc4c4ccccc34)cc2)c1 ; c1ccc(N(c2ccc(-c3cc4ccccc4c4ccccc34)cc2)c2cccc(-c3ccc4ccccc4c3)c2)cc1]

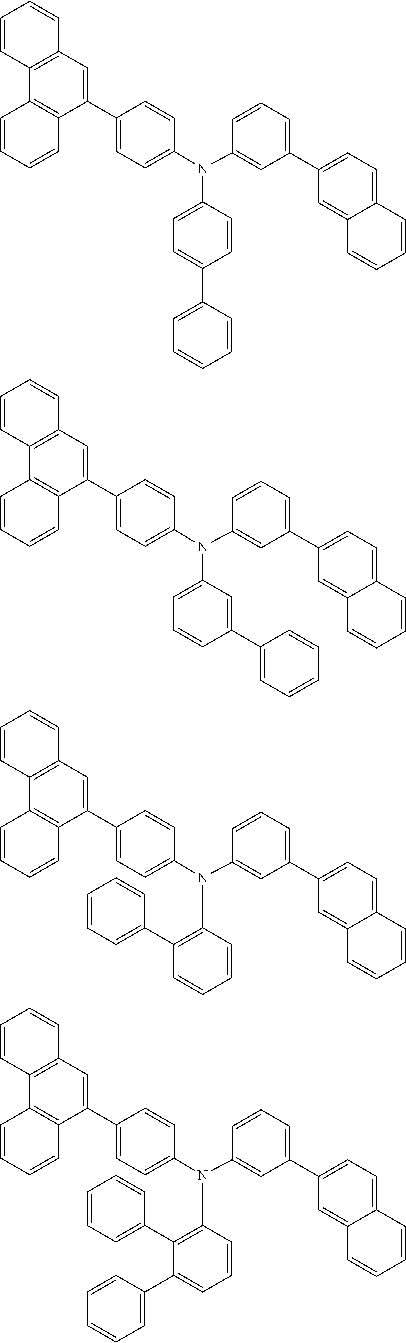 [SMILES: c1ccc(-c2ccc(N(c3ccc(-c4cc5ccccc5c5ccccc45)cc3)c3cccc(-c4ccc5ccccc5c4)c3)cc2)cc1 ; c1ccc(-c2cccc(N(c3ccc(-c4cc5ccccc5c5ccccc45)cc3)c3cccc(-c4ccc5ccccc5c4)c3)c2)cc1 ; c1ccc(-c2ccccc2N(c2ccc(-c3cc4ccccc4c4ccccc34)cc2)c2cccc(-c3ccc4ccccc4c3)c2)cc1 ; c1ccc(-c2cccc(N(c3ccc(-c4cc5ccccc5c5ccccc45)cc3)c3cccc(-c4ccc5ccccc5c4)c3)c2-c2ccccc2)cc1]

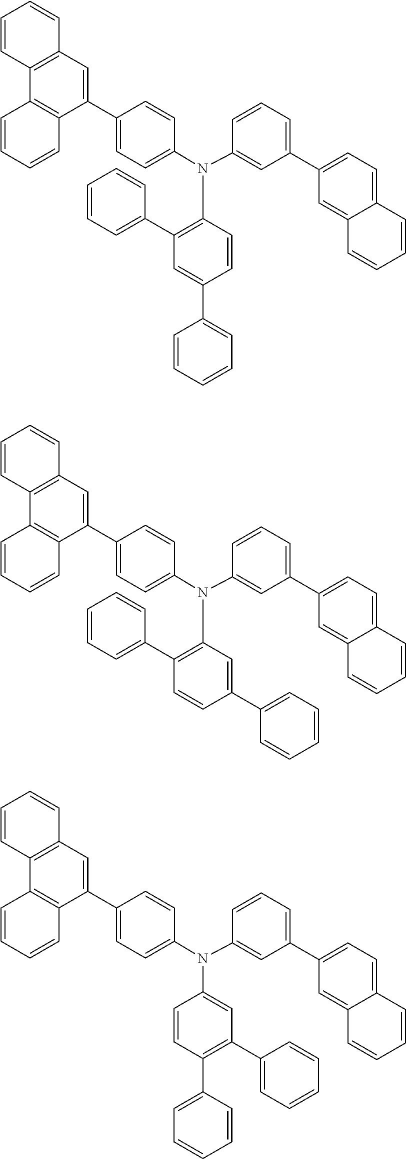 [SMILES: c1ccc(-c2ccc(N(c3ccc(-c4cc5ccccc5c5ccccc45)cc3)c3cccc(-c4ccc5ccccc5c4)c3)c(-c3ccccc3)c2)cc1 ; c1ccc(-c2ccc(-c3ccccc3)c(N(c3ccc(-c4cc5ccccc5c5ccccc45)cc3)c3cccc(-c4ccc5ccccc5c4)c3)c2)cc1 ; c1ccc(-c2ccc(N(c3ccc(-c4cc5ccccc5c5ccccc45)cc3)c3cccc(-c4ccc5ccccc5c4)c3)cc2-c2ccccc2)cc1]

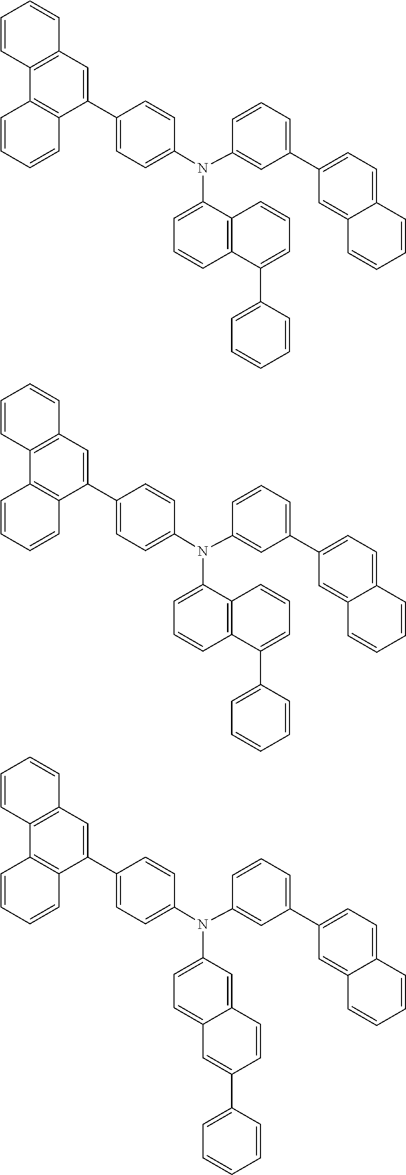 [SMILES: c1ccc(-c2cccc3c(N(c4ccc(-c5cc6ccccc6c6ccccc56)cc4)c4cccc(-c5ccc6ccccc6c5)c4)cccc23)cc1 ; c1ccc(-c2cccc3c(N(c4ccc(-c5cc6ccccc6c6ccccc56)cc4)c4cccc(-c5ccc6ccccc6c5)c4)cccc23)cc1 ; c1ccc(-c2ccc3cc(N(c4ccc(-c5cc6ccccc6c6ccccc56)cc4)c4cccc(-c5ccc6ccccc6c5)c4)ccc3c2)cc1]

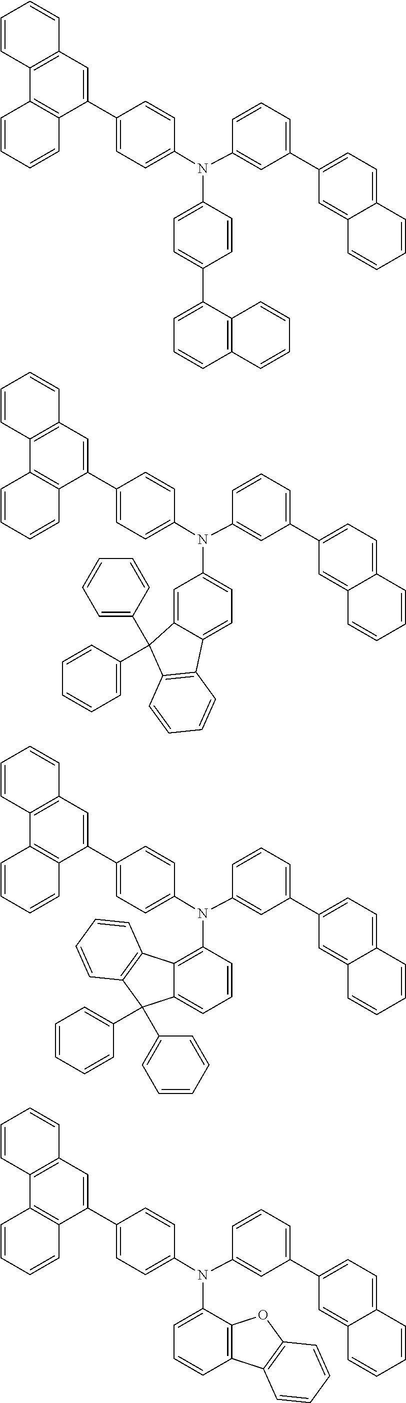 [SMILES: c1cc(-c2ccc3ccccc3c2)cc(N(c2ccc(-c3cccc4ccccc34)cc2)c2ccc(-c3cc4ccccc4c4ccccc34)cc2)c1 ; c1ccc(C2(c3ccccc3)c3ccccc3-c3ccc(N(c4ccc(-c5cc6ccccc6c6ccccc56)cc4)c4cccc(-c5ccc6ccccc6c5)c4)cc32)cc1 ; c1ccc(C2(c3ccccc3)c3ccccc3-c3c(N(c4ccc(-c5cc6ccccc6c6ccccc56)cc4)c4cccc(-c5ccc6ccccc6c5)c4)cccc32)cc1 ; c1cc(-c2ccc3ccccc3c2)cc(N(c2ccc(-c3cc4ccccc4c4ccccc34)cc2)c2cccc3c2oc2ccccc23)c1]

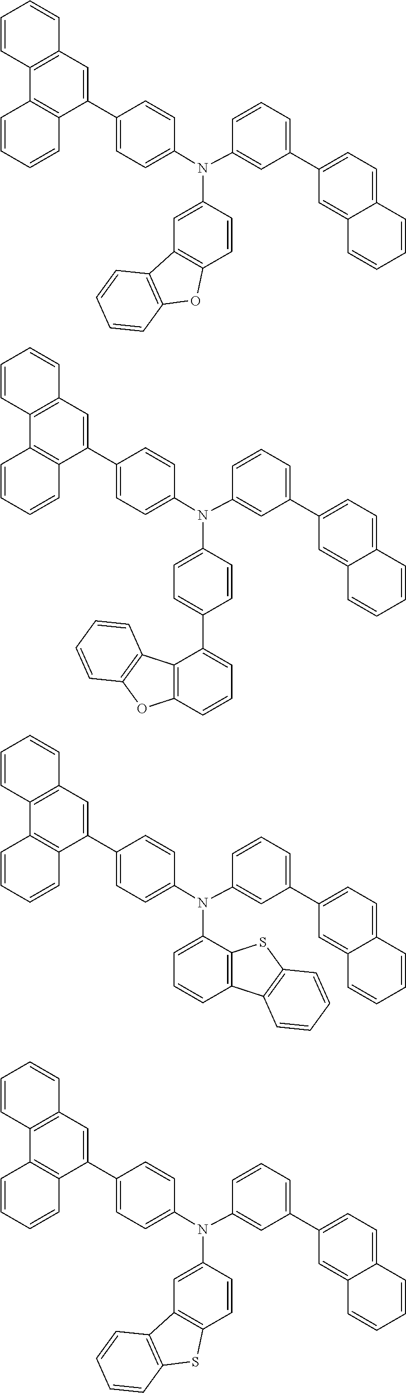 [SMILES: c1cc(-c2ccc3ccccc3c2)cc(N(c2ccc(-c3cc4ccccc4c4ccccc34)cc2)c2ccc3oc4ccccc4c3c2)c1 ; c1cc(-c2ccc3ccccc3c2)cc(N(c2ccc(-c3cc4ccccc4c4ccccc34)cc2)c2ccc(-c3cccc4oc5ccccc5c34)cc2)c1 ; c1cc(-c2ccc3ccccc3c2)cc(N(c2ccc(-c3cc4ccccc4c4ccccc34)cc2)c2cccc3c2sc2ccccc23)c1 ; c1cc(-c2ccc3ccccc3c2)cc(N(c2ccc(-c3cc4ccccc4c4ccccc34)cc2)c2ccc3sc4ccccc4c3c2)c1]

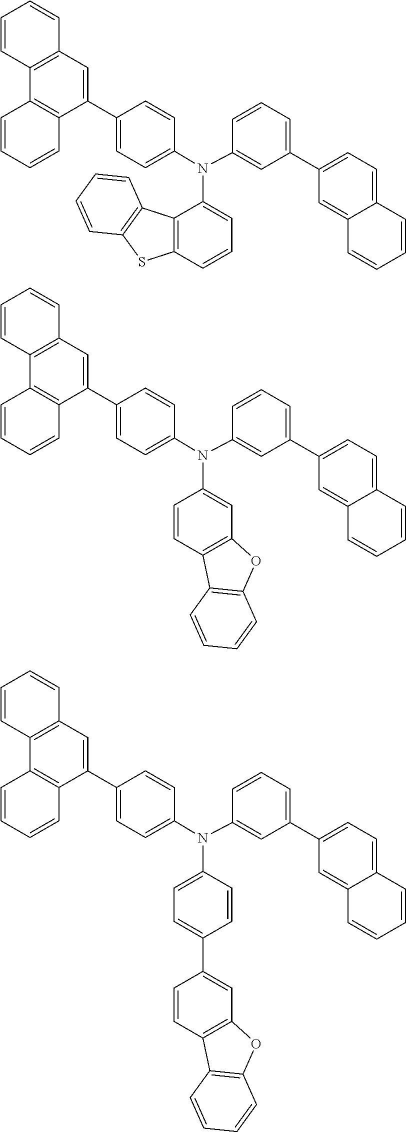 [SMILES: c1cc(-c2ccc3ccccc3c2)cc(N(c2ccc(-c3cc4ccccc4c4ccccc34)cc2)c2cccc3sc4ccccc4c23)c1 ; c1cc(-c2ccc3ccccc3c2)cc(N(c2ccc(-c3cc4ccccc4c4ccccc34)cc2)c2ccc3c(c2)oc2ccccc23)c1 ; c1cc(-c2ccc3ccccc3c2)cc(N(c2ccc(-c3ccc4c(c3)oc3ccccc34)cc2)c2ccc(-c3cc4ccccc4c4ccccc34)cc2)c1]

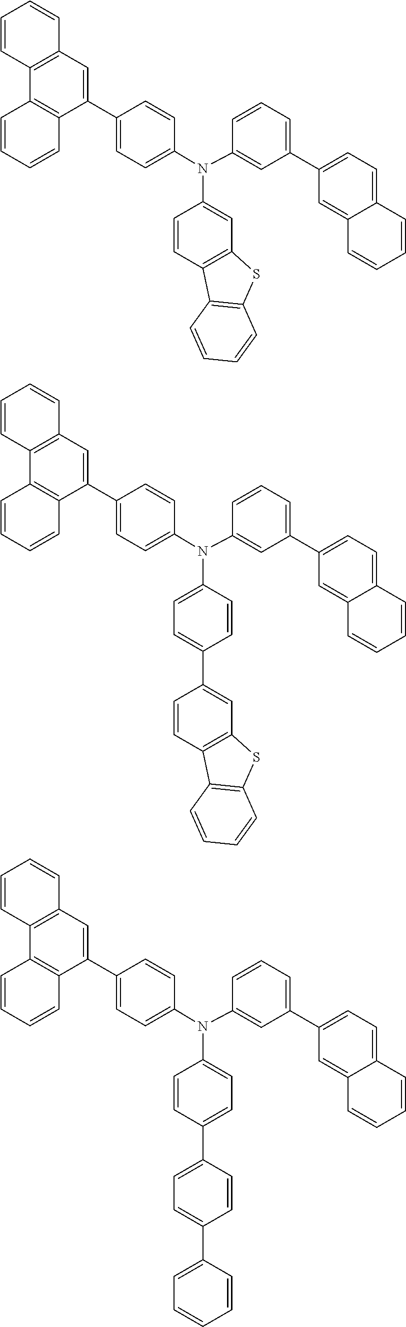 [SMILES: c1cc(-c2ccc3ccccc3c2)cc(N(c2ccc(-c3cc4ccccc4c4ccccc34)cc2)c2ccc3c(c2)sc2ccccc23)c1 ; c1cc(-c2ccc3ccccc3c2)cc(N(c2ccc(-c3ccc4c(c3)sc3ccccc34)cc2)c2ccc(-c3cc4ccccc4c4ccccc34)cc2)c1 ; c1ccc(-c2ccc(-c3ccc(N(c4ccc(-c5cc6ccccc6c6ccccc56)cc4)c4cccc(-c5ccc6ccccc6c5)c4)cc3)cc2)cc1]

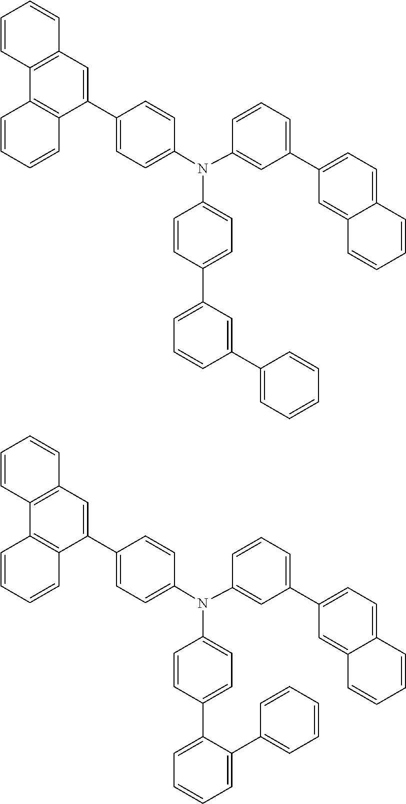 [SMILES: c1ccc(-c2cccc(-c3ccc(N(c4ccc(-c5cc6ccccc6c6ccccc56)cc4)c4cccc(-c5ccc6ccccc6c5)c4)cc3)c2)cc1 ; c1ccc(-c2ccccc2-c2ccc(N(c3ccc(-c4cc5ccccc5c5ccccc45)cc3)c3cccc(-c4ccc5ccccc5c4)c3)cc2)cc1]

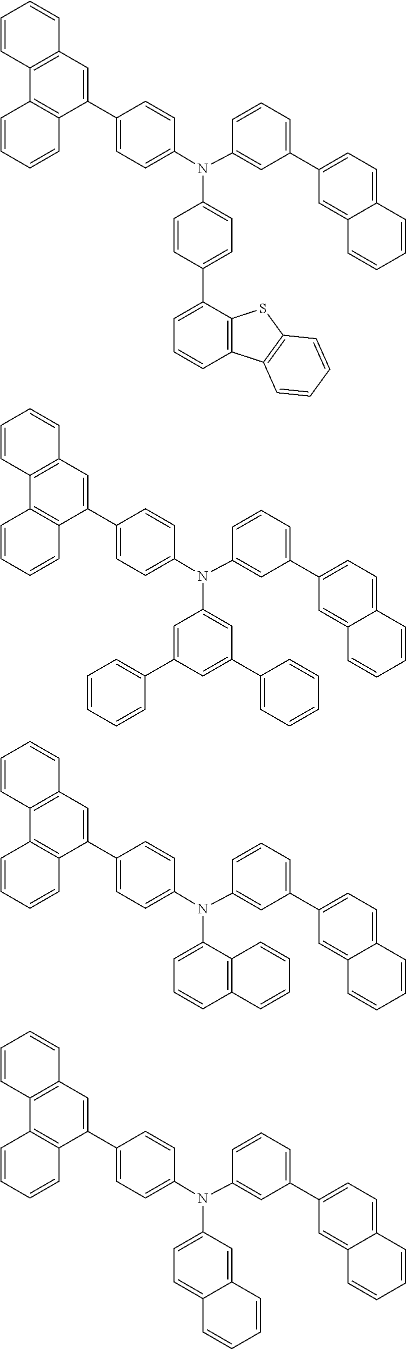 [SMILES: c1cc(-c2ccc3ccccc3c2)cc(N(c2ccc(-c3cc4ccccc4c4ccccc34)cc2)c2ccc(-c3cccc4c3sc3ccccc34)cc2)c1 ; c1ccc(-c2cc(-c3ccccc3)cc(N(c3ccc(-c4cc5ccccc5c5ccccc45)cc3)c3cccc(-c4ccc5ccccc5c4)c3)c2)cc1 ; c1cc(-c2ccc3ccccc3c2)cc(N(c2ccc(-c3cc4ccccc4c4ccccc34)cc2)c2cccc3ccccc23)c1 ; c1cc(-c2ccc3ccccc3c2)cc(N(c2ccc(-c3cc4ccccc4c4ccccc34)cc2)c2ccc3ccccc3c2)c1]

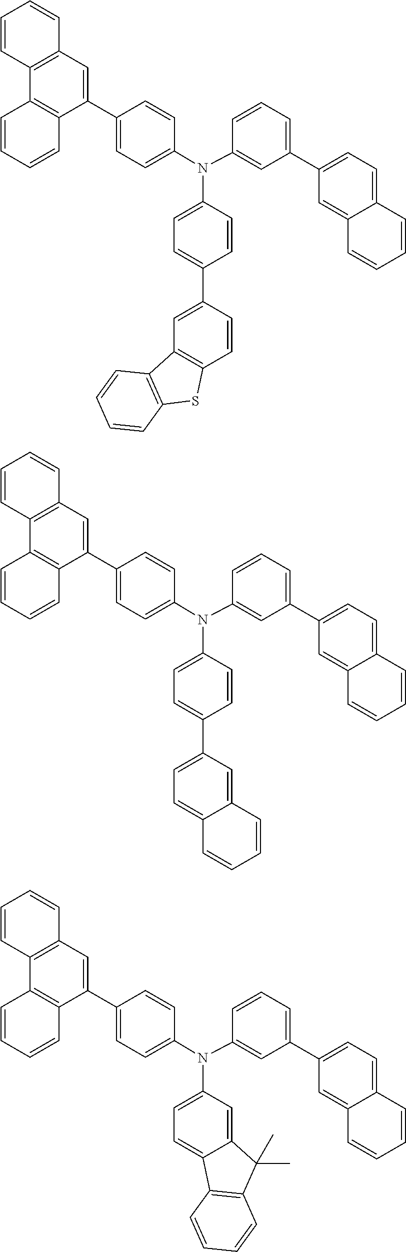 [SMILES: c1cc(-c2ccc3ccccc3c2)cc(N(c2ccc(-c3ccc4sc5ccccc5c4c3)cc2)c2ccc(-c3cc4ccccc4c4ccccc34)cc2)c1 ; c1cc(-c2ccc3ccccc3c2)cc(N(c2ccc(-c3ccc4ccccc4c3)cc2)c2ccc(-c3cc4ccccc4c4ccccc34)cc2)c1 ; CC1(C)c2ccccc2-c2ccc(N(c3ccc(-c4cc5ccccc5c5ccccc45)cc3)c3cccc(-c4ccc5ccccc5c4)c3)cc21]

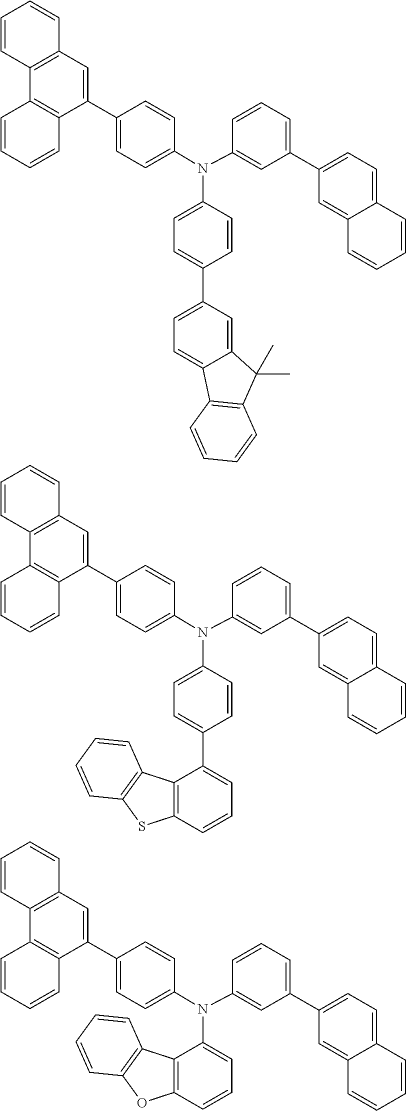 [SMILES: CC1(C)c2ccccc2-c2ccc(-c3ccc(N(c4ccc(-c5cc6ccccc6c6ccccc56)cc4)c4cccc(-c5ccc6ccccc6c5)c4)cc3)cc21 ; c1cc(-c2ccc3ccccc3c2)cc(N(c2ccc(-c3cc4ccccc4c4ccccc34)cc2)c2ccc(-c3cccc4sc5ccccc5c34)cc2)c1 ; c1cc(-c2ccc3ccccc3c2)cc(N(c2ccc(-c3cc4ccccc4c4ccccc34)cc2)c2cccc3oc4ccccc4c23)c1]

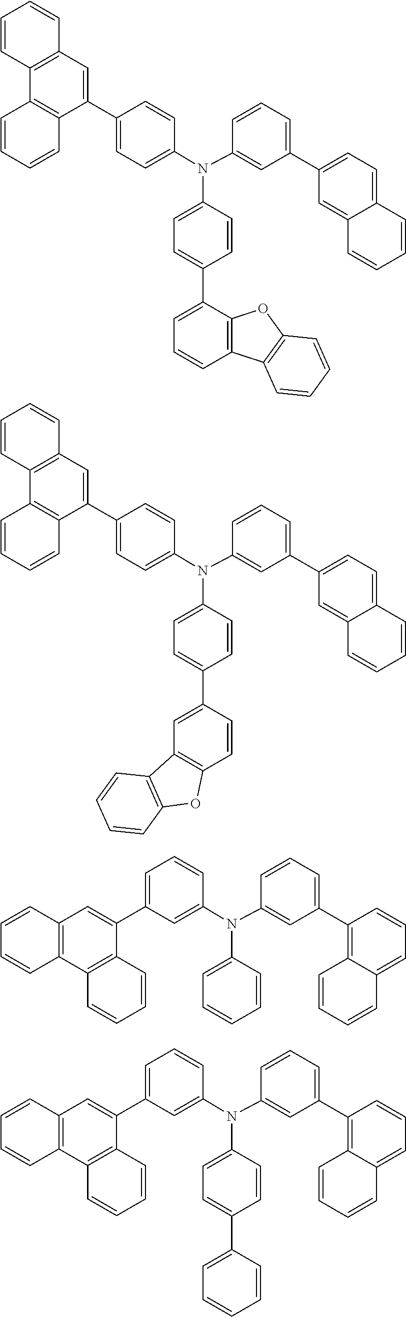

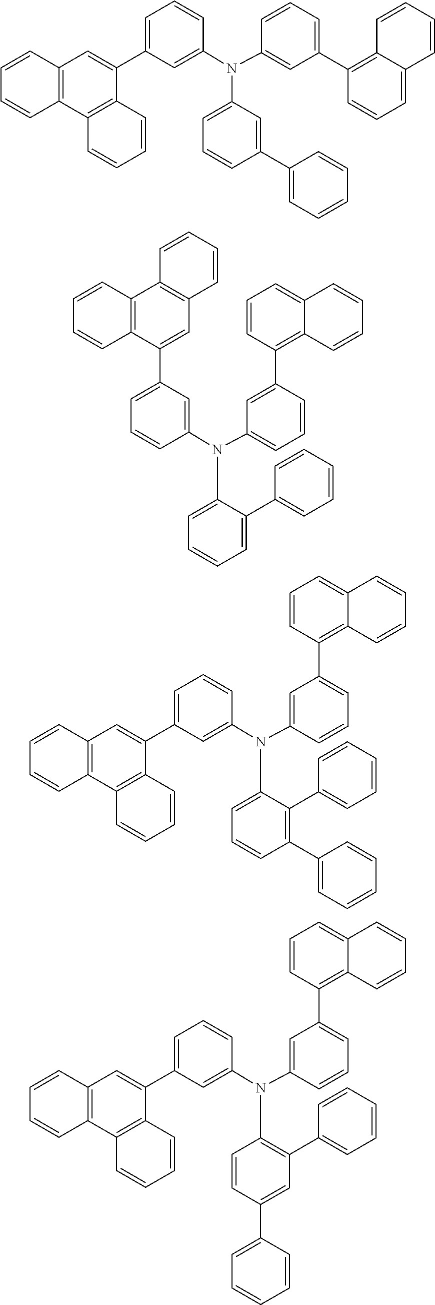

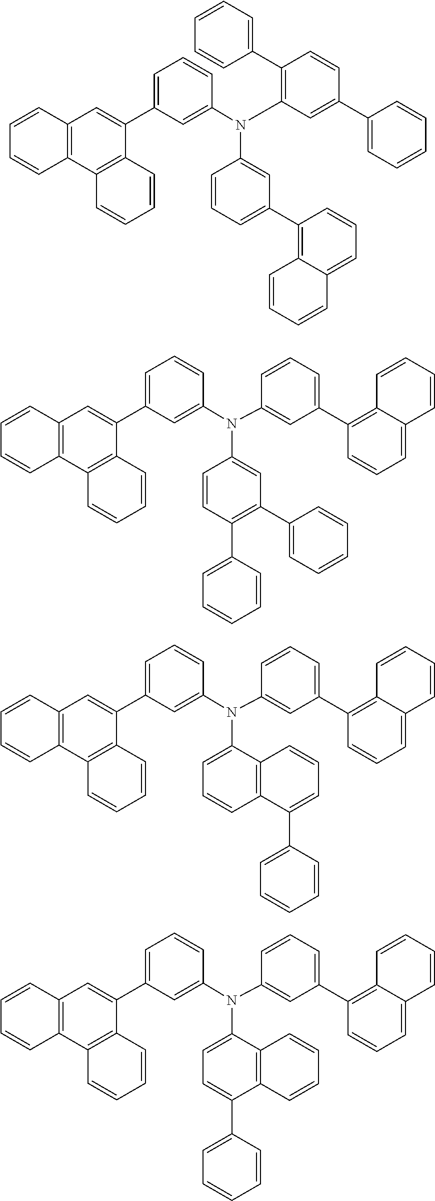

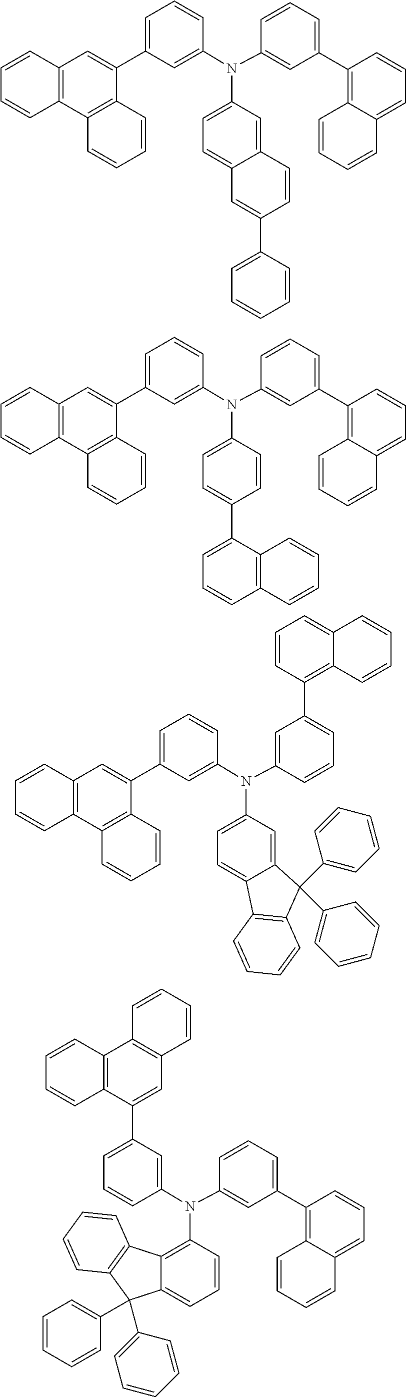

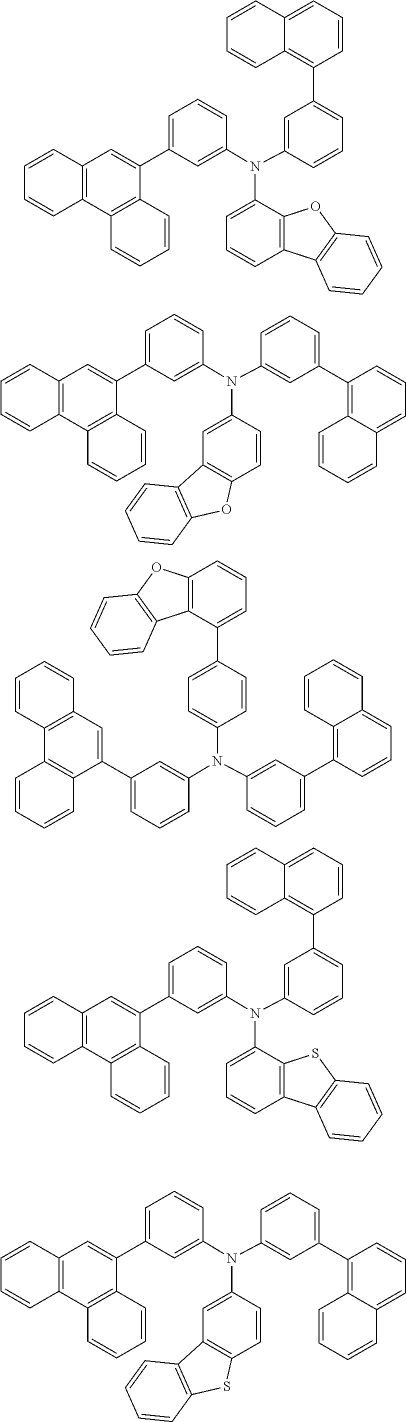

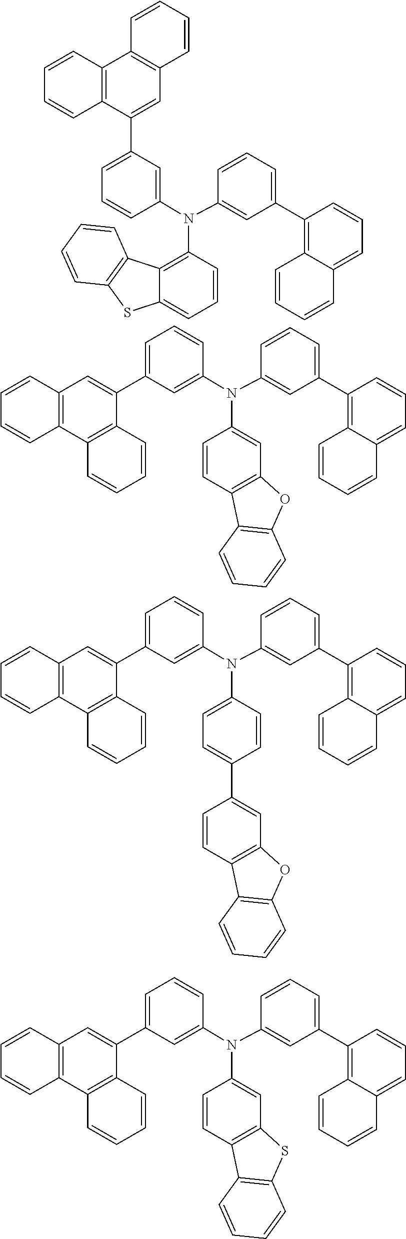

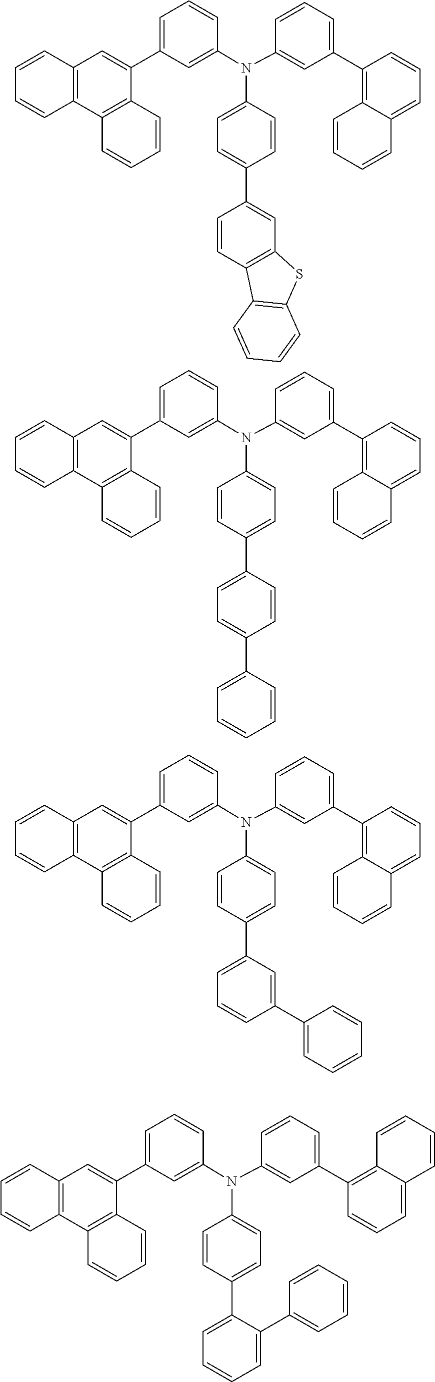

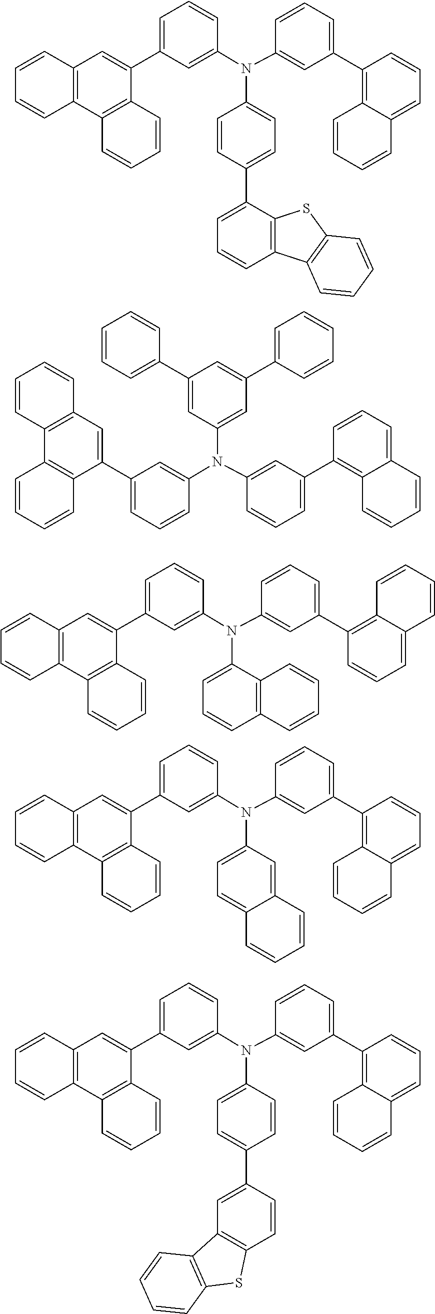

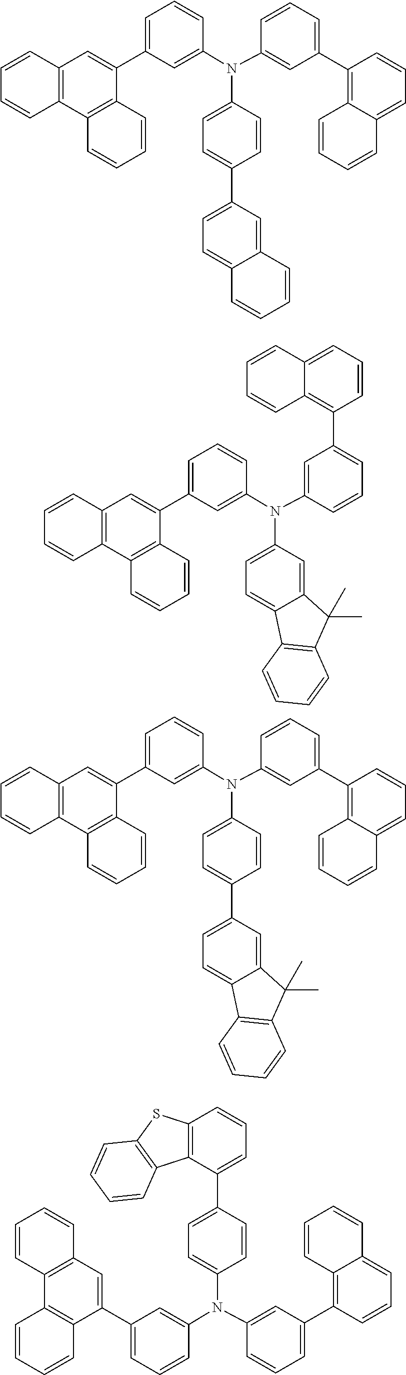

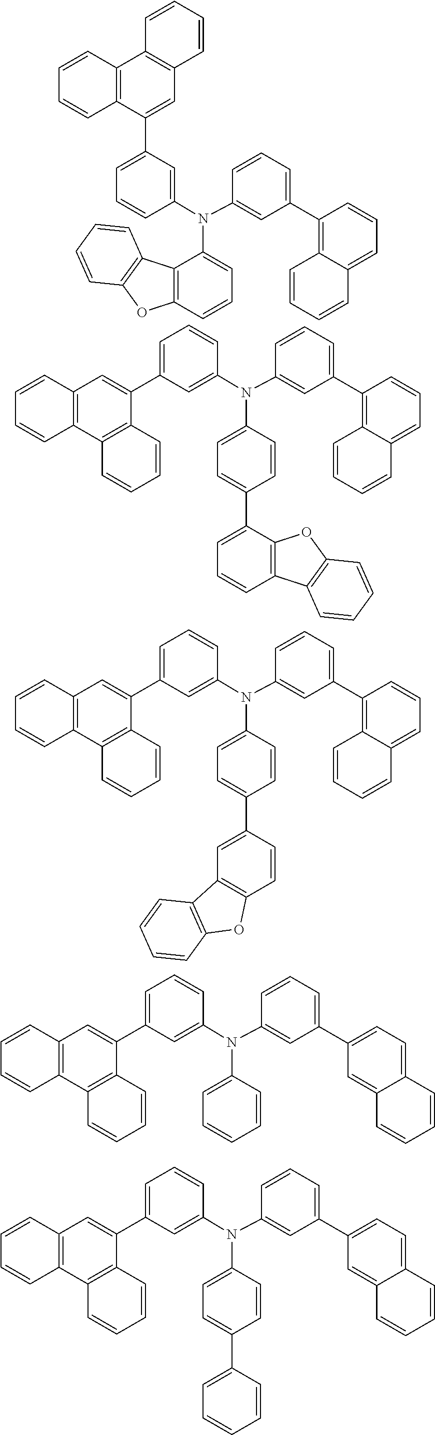

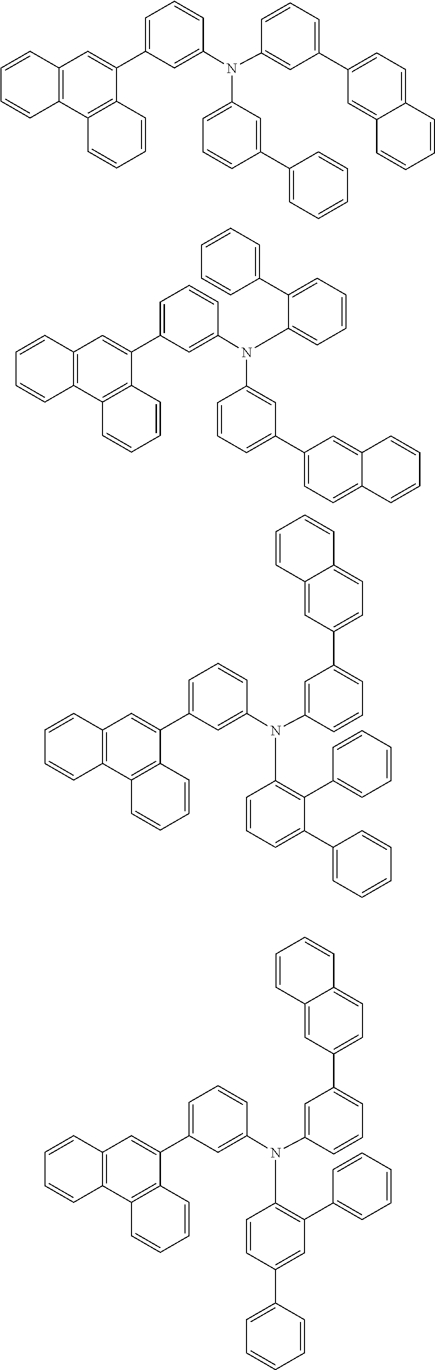

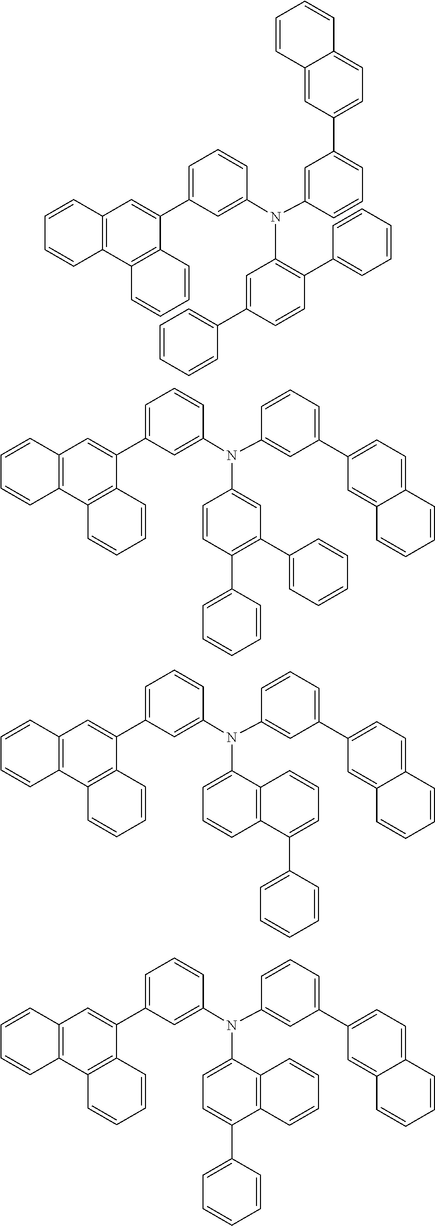

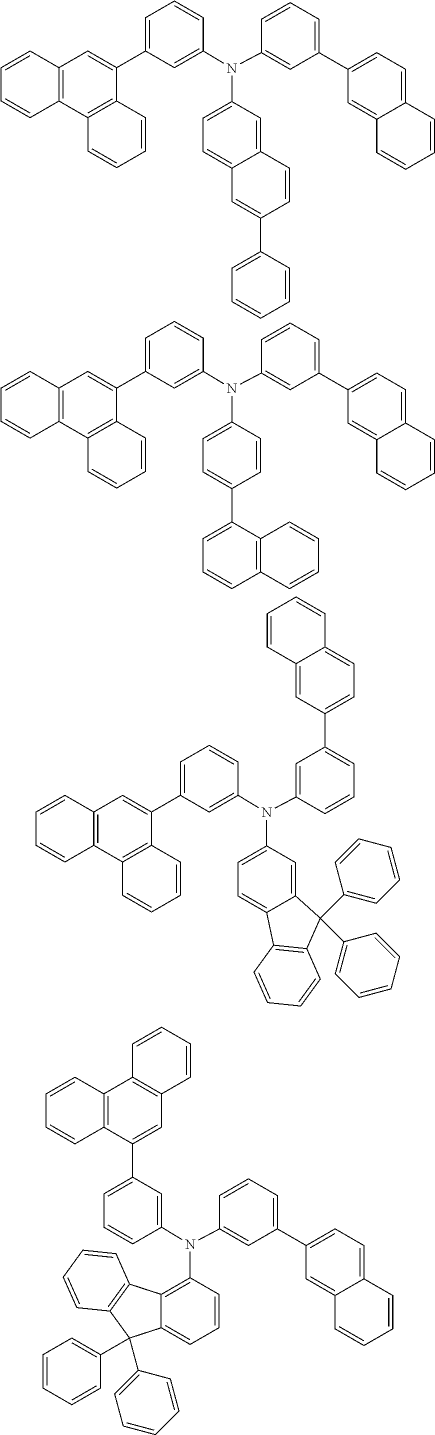

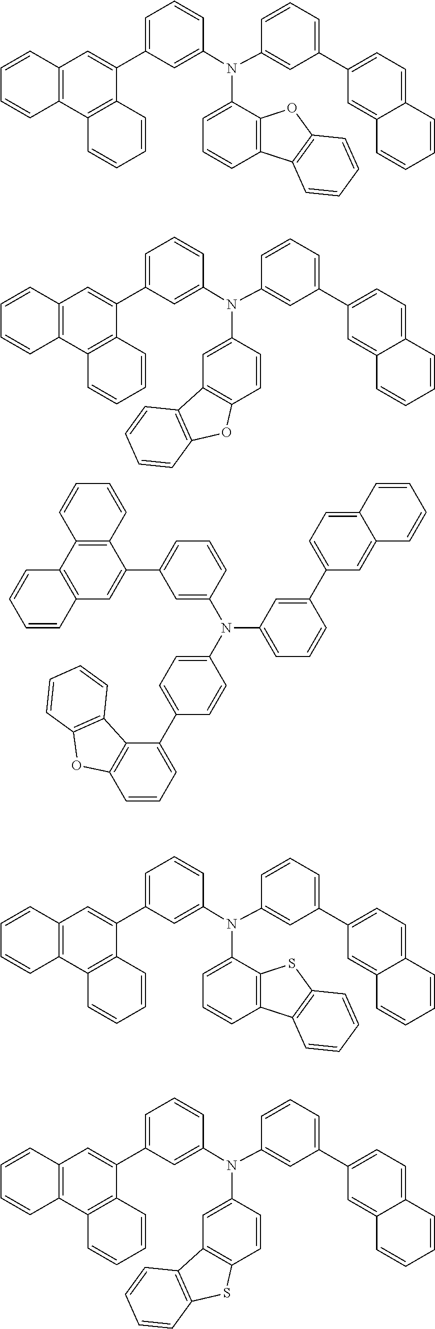

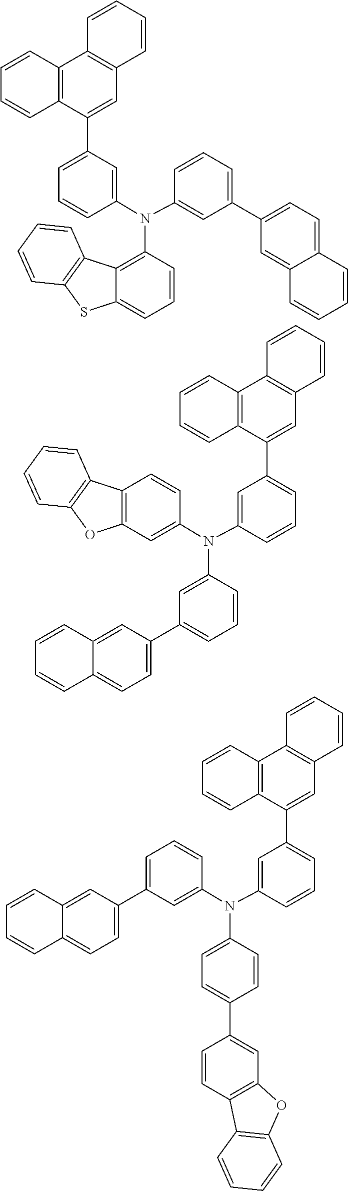

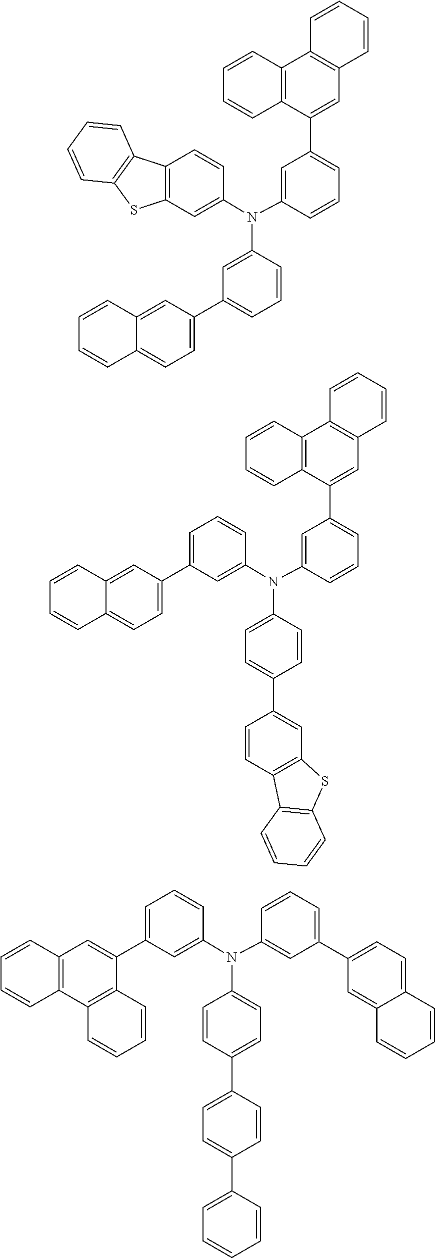

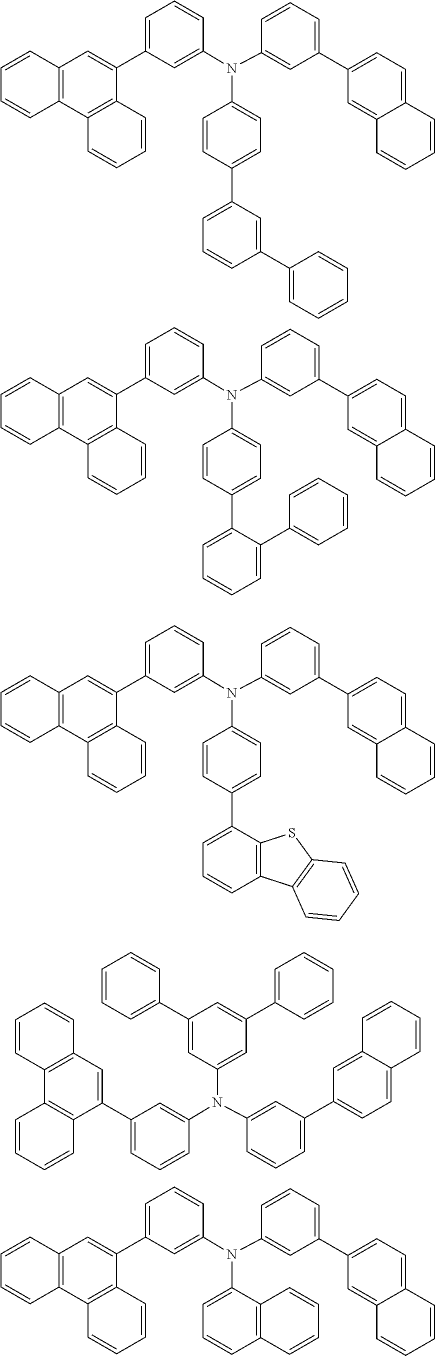

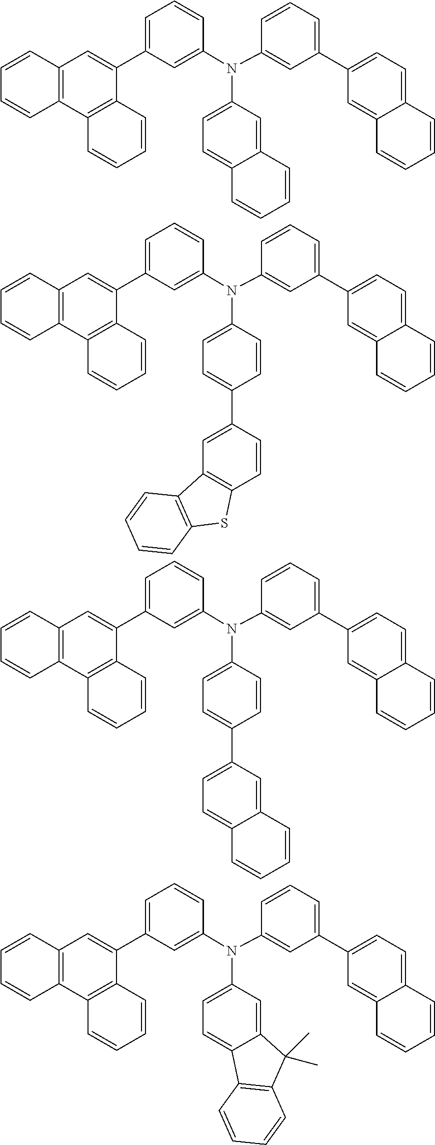

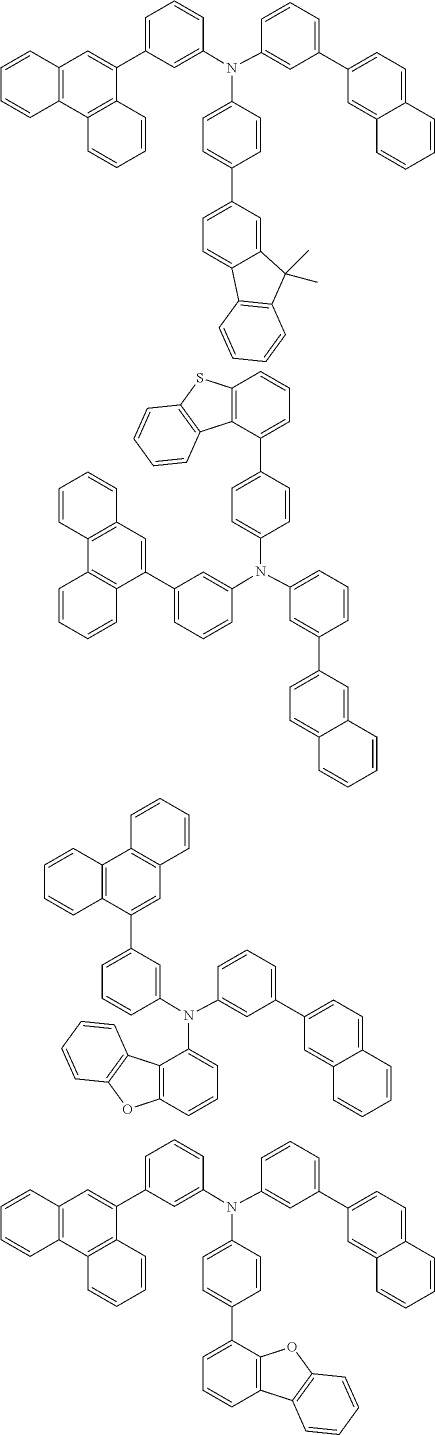

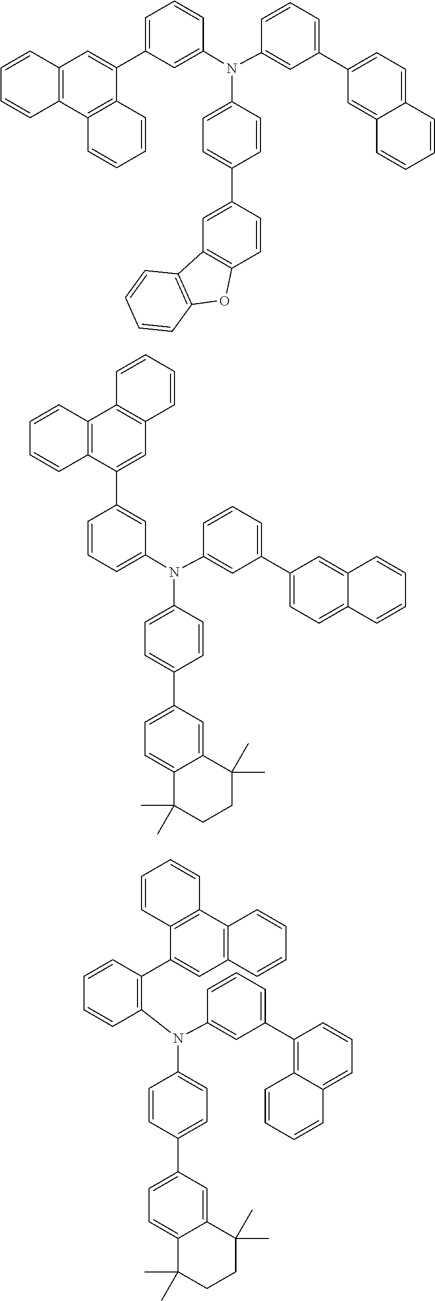

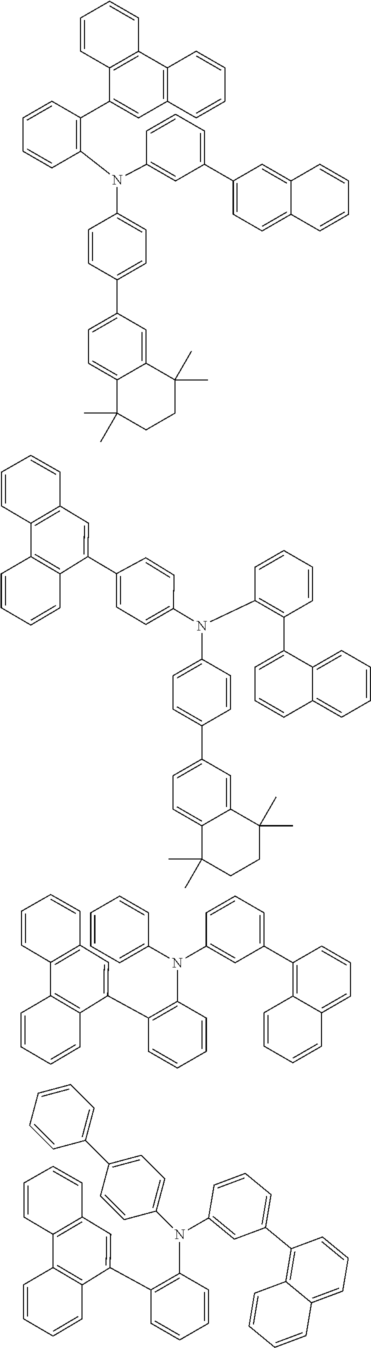

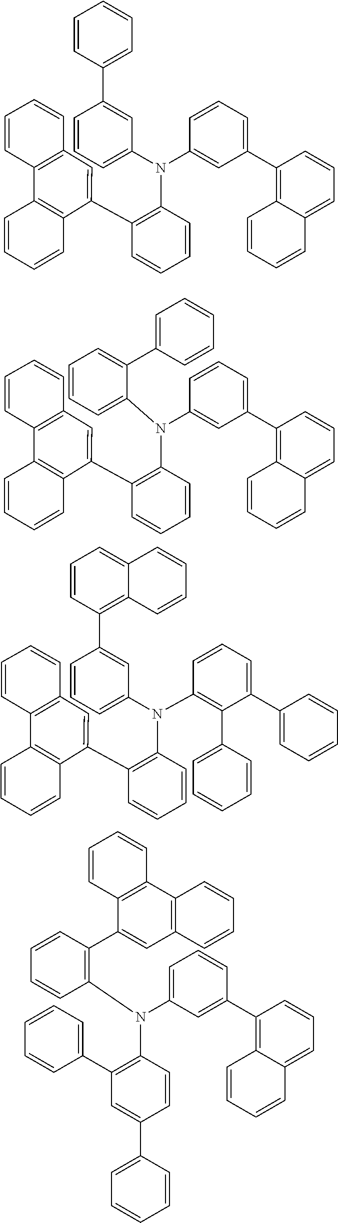

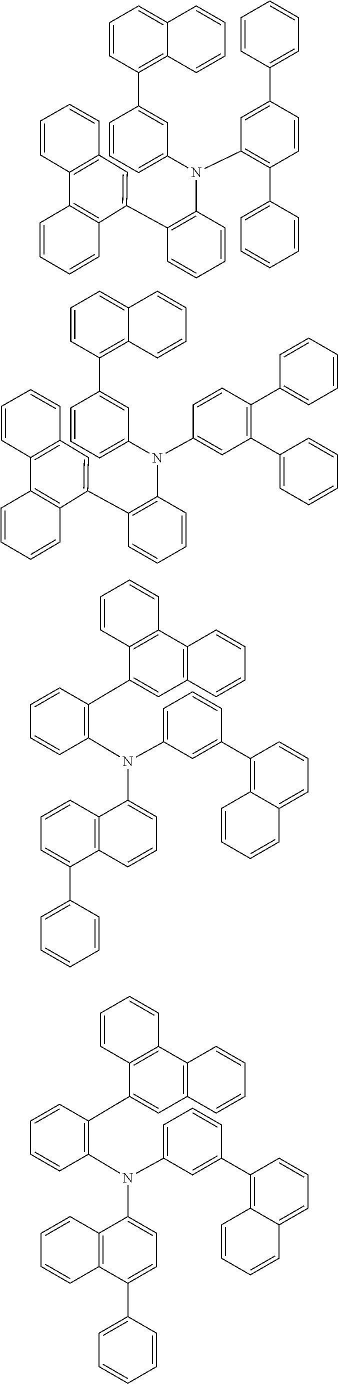

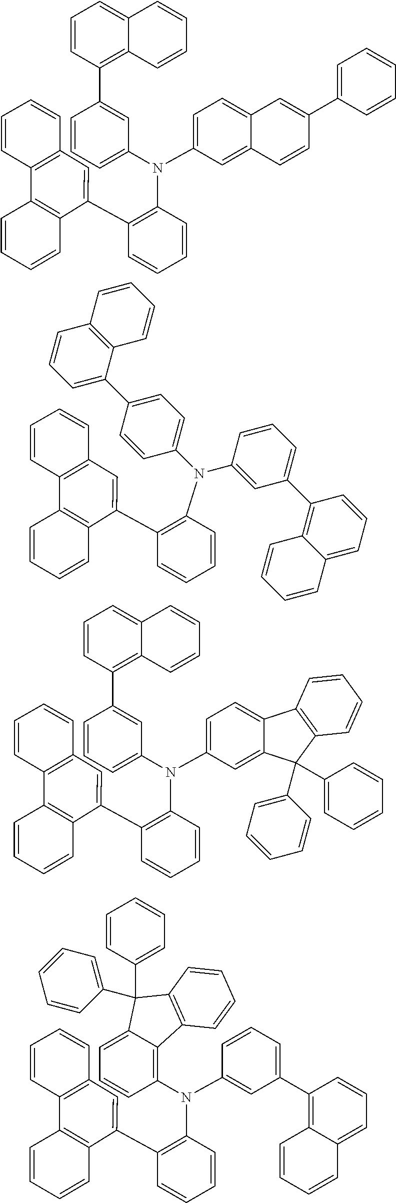

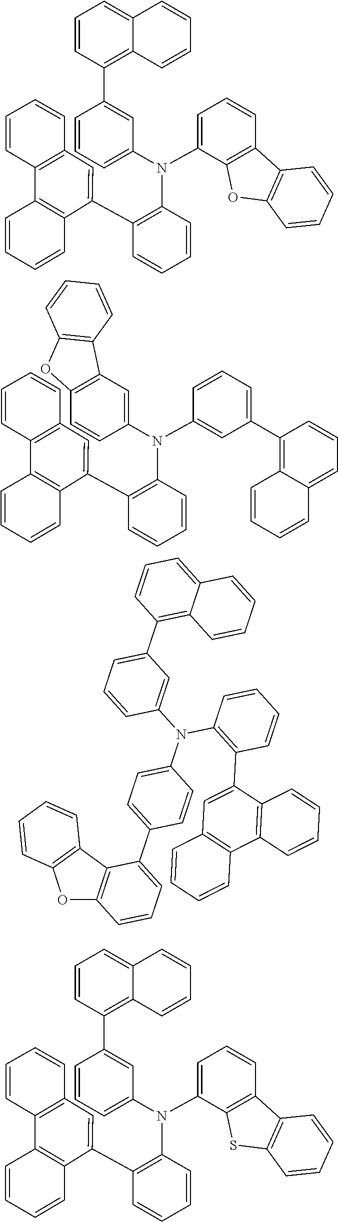

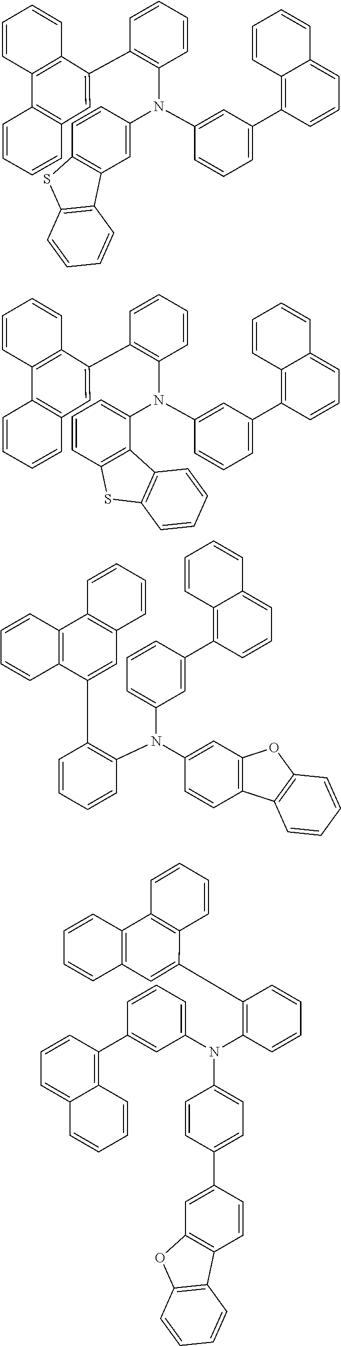

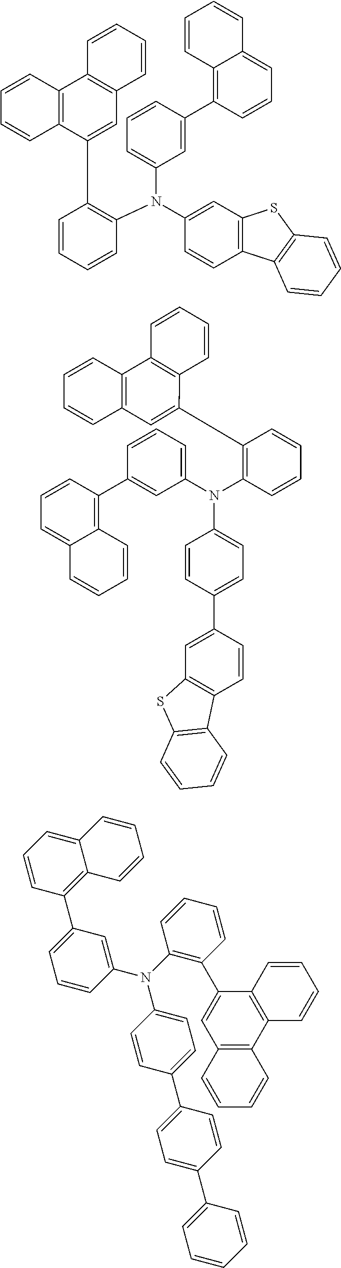 [SMILES: c1cc(-c2cccc3ccccc23)cc(N(c2ccc3c(c2)sc2ccccc23)c2ccccc2-c2cc3ccccc3c3ccccc23)c1 ; c1cc(-c2cccc3ccccc23)cc(N(c2ccc(-c3ccc4c(c3)sc3ccccc34)cc2)c2ccccc2-c2cc3ccccc3c3ccccc23)c1 ; c1ccc(-c2ccc(-c3ccc(N(c4cccc(-c5cccc6ccccc56)c4)c4ccccc4-c4cc5ccccc5c5ccccc45)cc3)cc2)cc1]

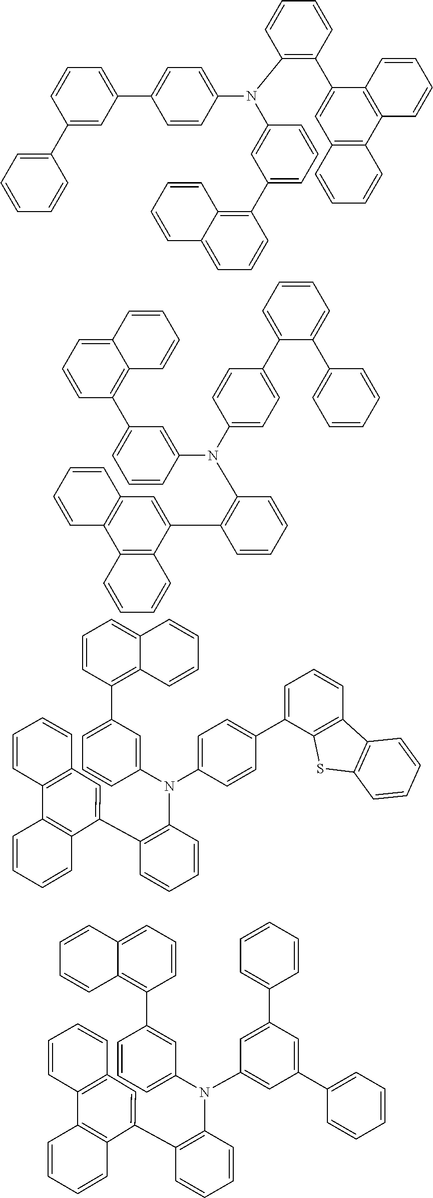

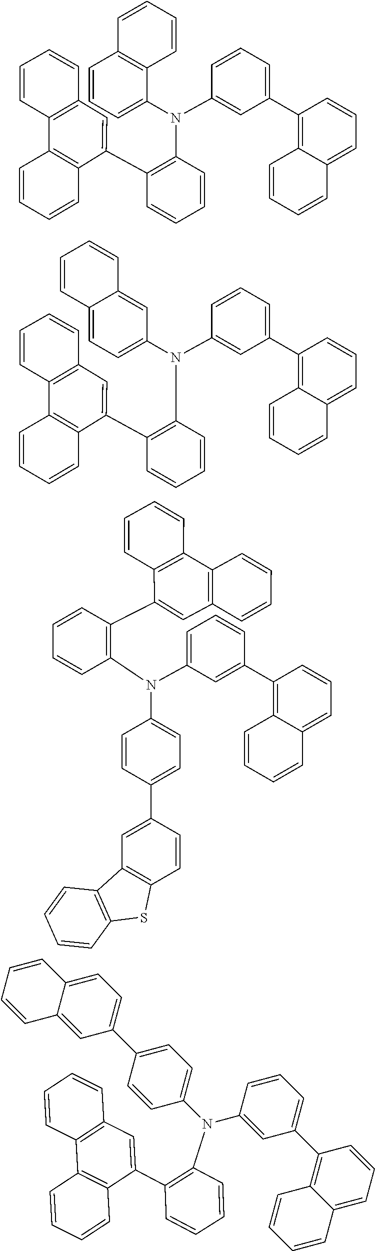

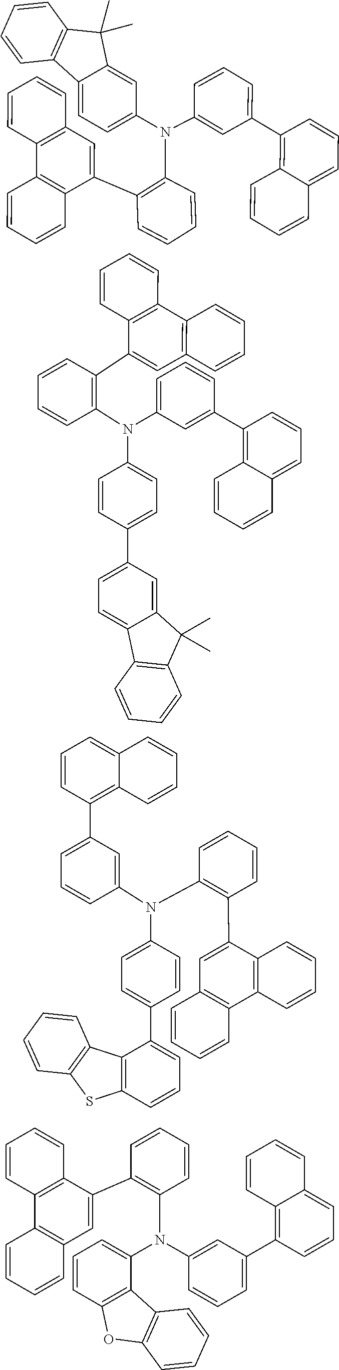

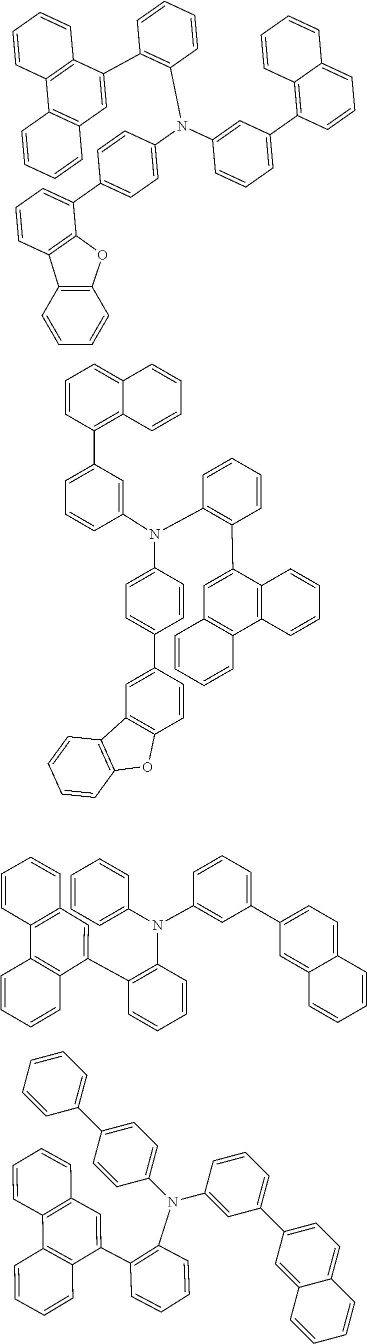

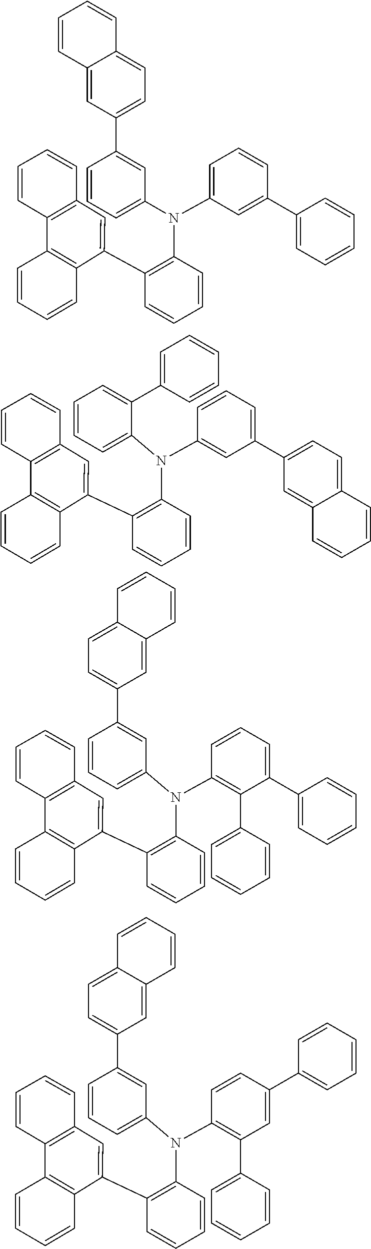

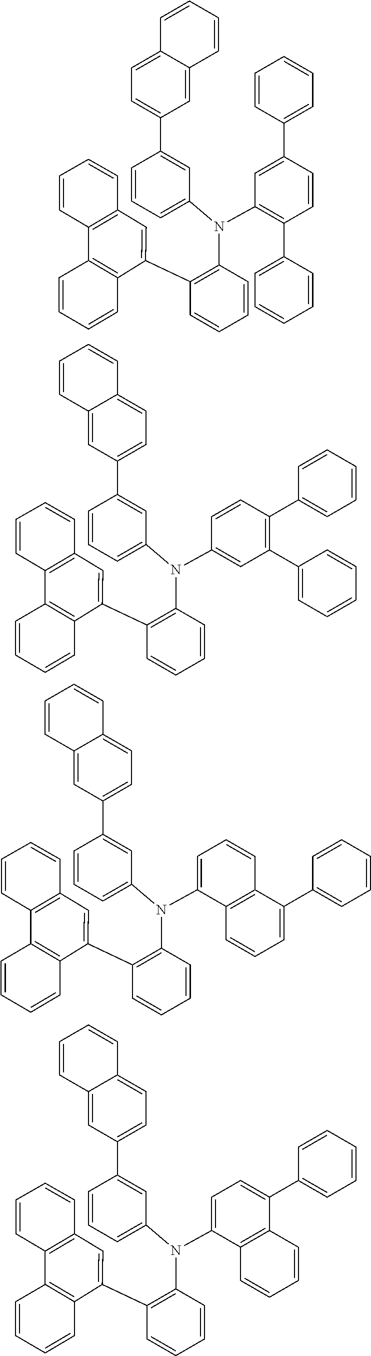

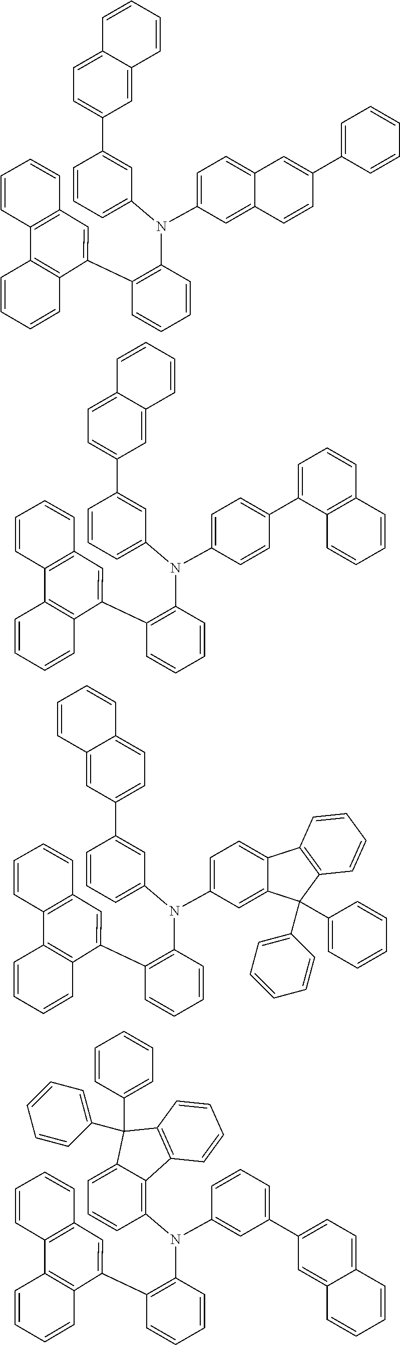

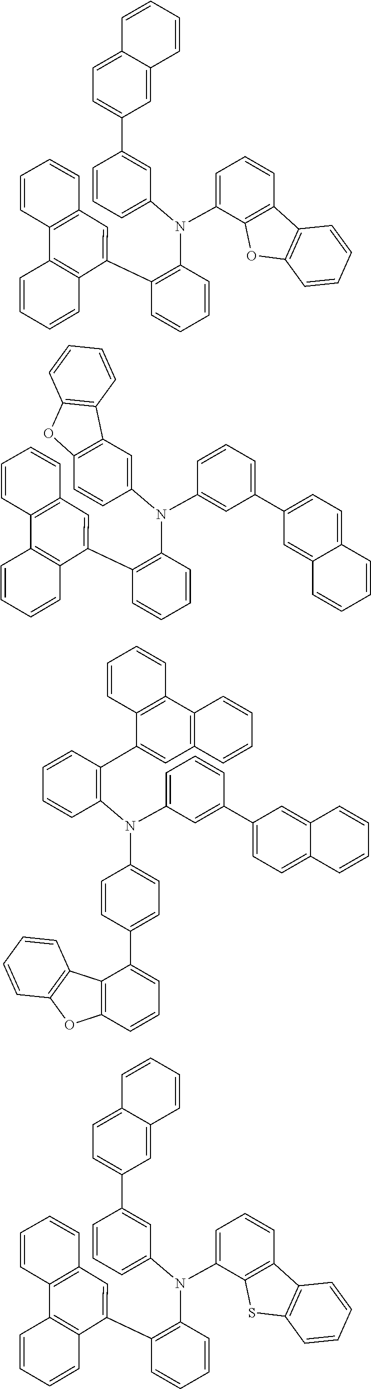

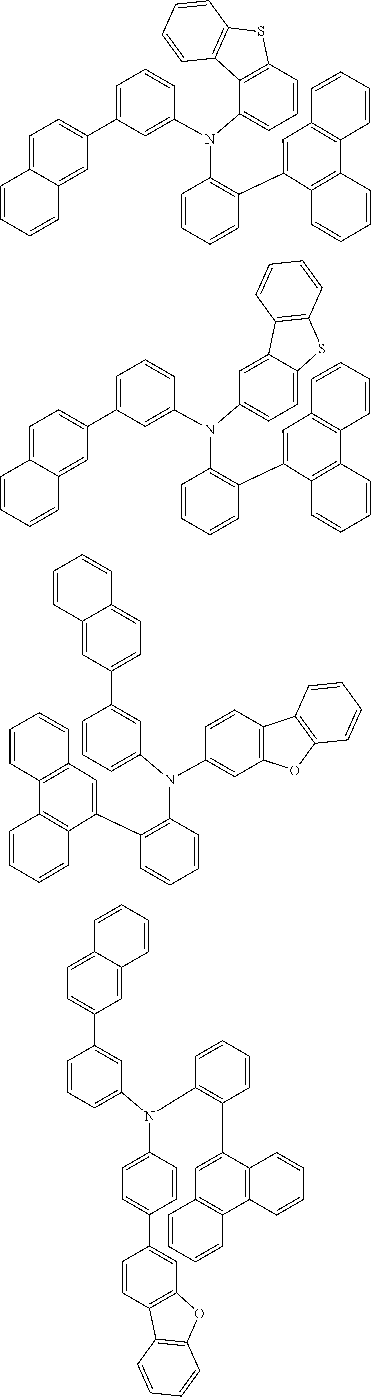

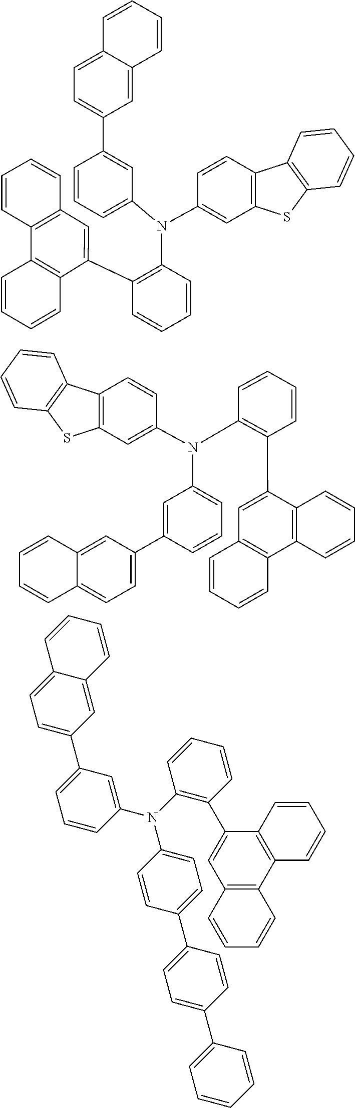

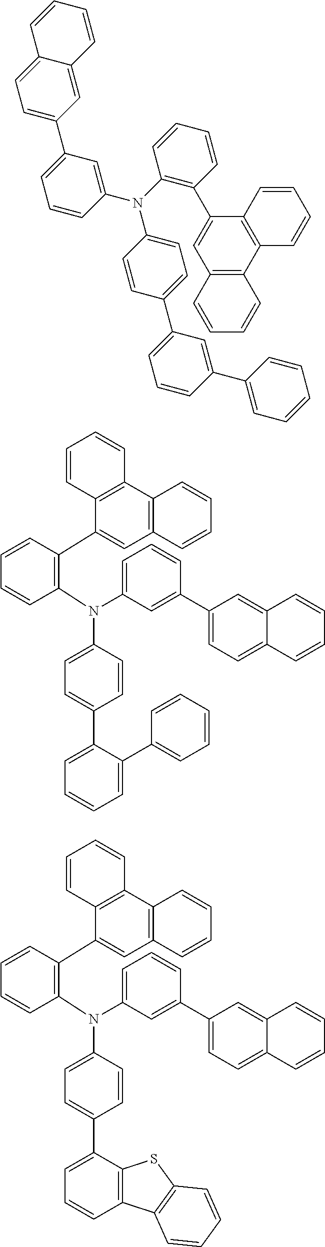

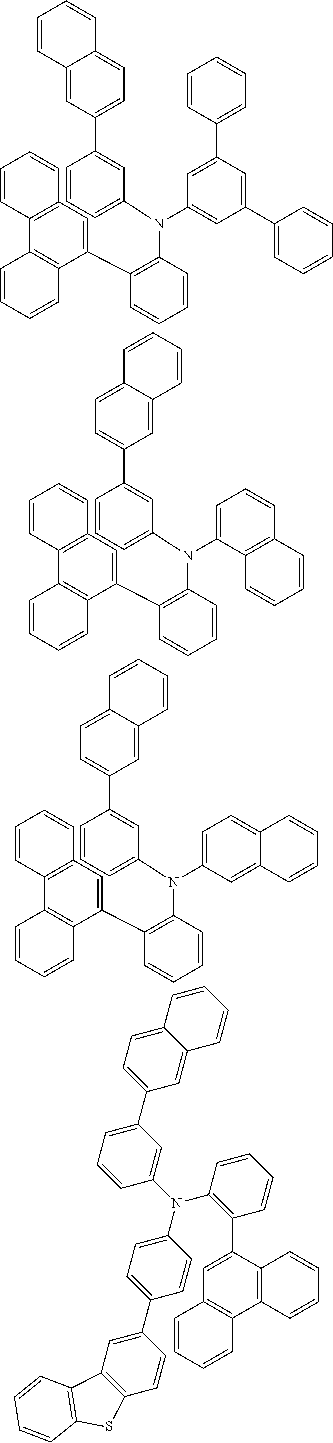

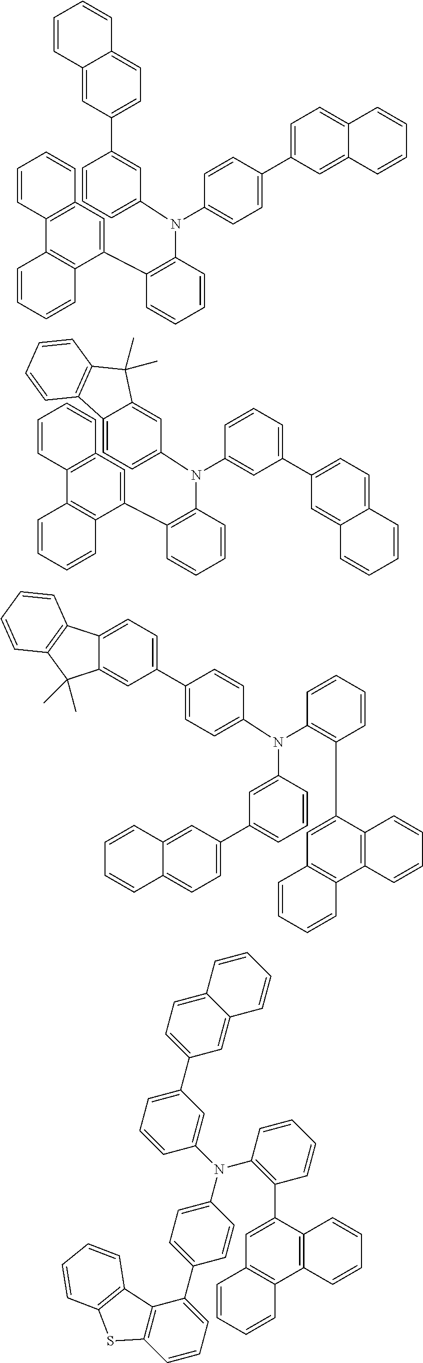

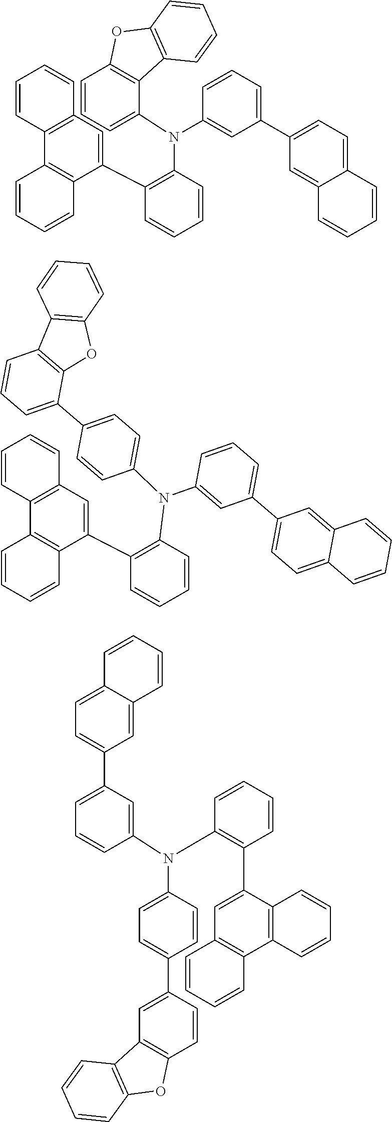

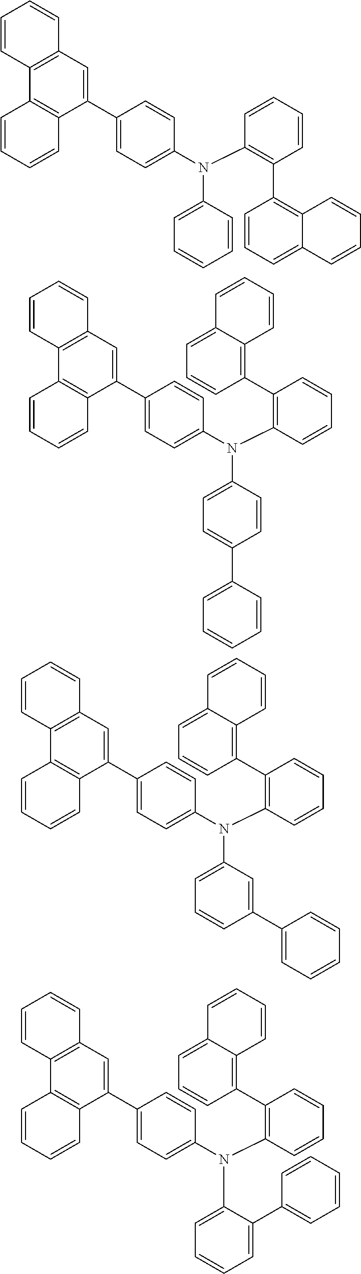

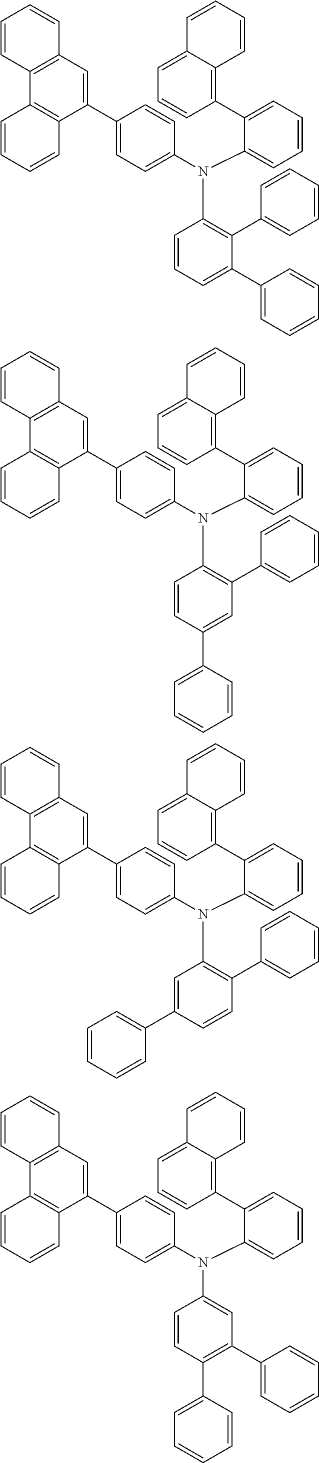

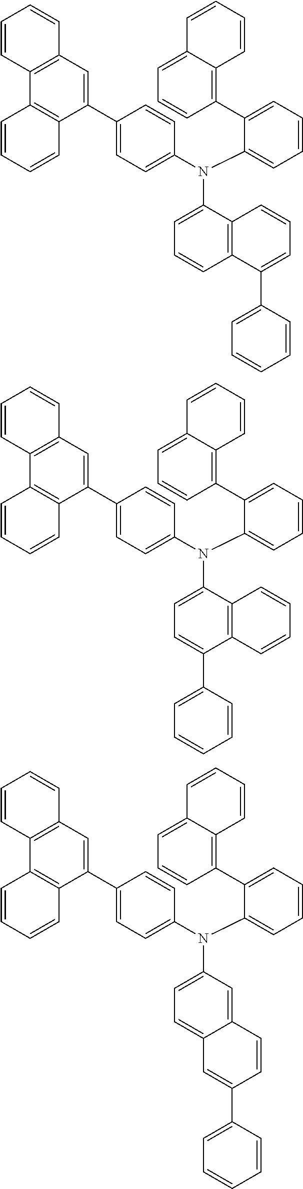

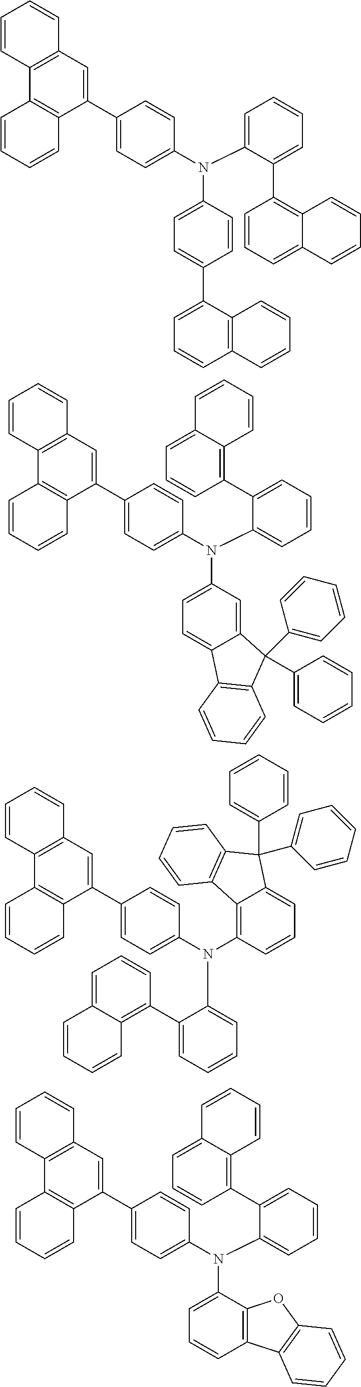

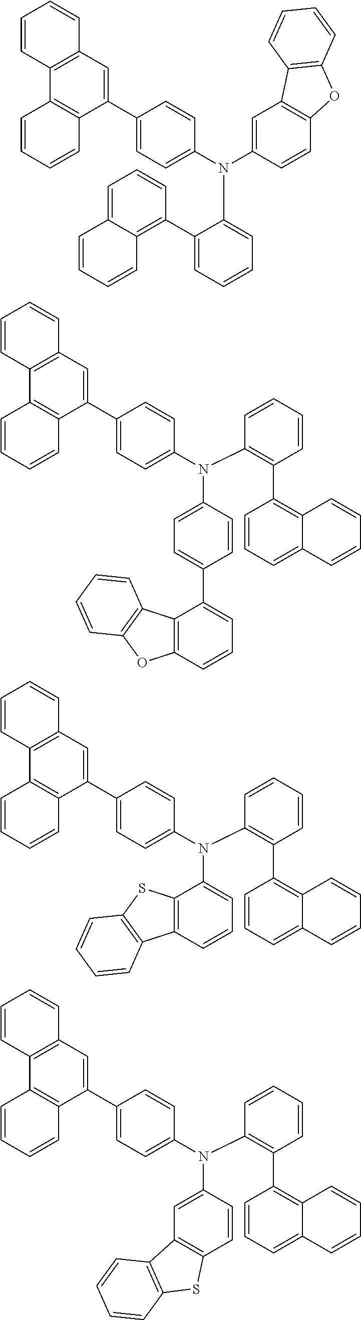 [SMILES: c1ccc(N(c2ccc(-c3cc4ccccc4c4ccccc34)cc2)c2ccc3oc4ccccc4c3c2)c(-c2cccc3ccccc23)c1 ; c1ccc(N(c2ccc(-c3cc4ccccc4c4ccccc34)cc2)c2ccc(-c3cccc4oc5ccccc5c34)cc2)c(-c2cccc3ccccc23)c1 ; c1ccc(N(c2ccc(-c3cc4ccccc4c4ccccc34)cc2)c2cccc3c2sc2ccccc23)c(-c2cccc3ccccc23)c1 ; c1ccc(N(c2ccc(-c3cc4ccccc4c4ccccc34)cc2)c2ccc3sc4ccccc4c3c2)c(-c2cccc3ccccc23)c1]

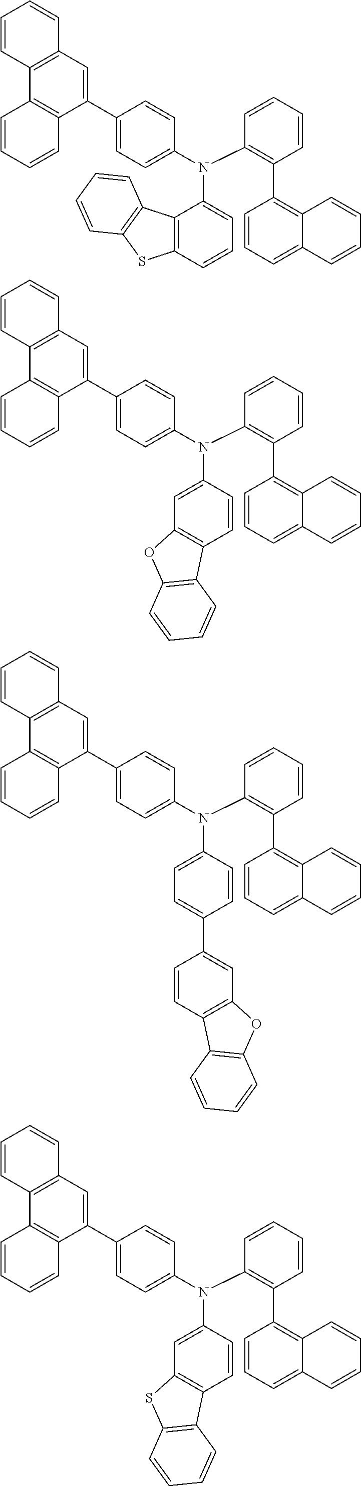 [SMILES: c1ccc(N(c2ccc(-c3cc4ccccc4c4ccccc34)cc2)c2cccc3sc4ccccc4c23)c(-c2cccc3ccccc23)c1 ; c1ccc(N(c2ccc(-c3cc4ccccc4c4ccccc34)cc2)c2ccc3c(c2)oc2ccccc23)c(-c2cccc3ccccc23)c1 ; c1ccc(N(c2ccc(-c3ccc4c(c3)oc3ccccc34)cc2)c2ccc(-c3cc4ccccc4c4ccccc34)cc2)c(-c2cccc3ccccc23)c1 ; c1ccc(N(c2ccc(-c3cc4ccccc4c4ccccc34)cc2)c2ccc3c(c2)sc2ccccc23)c(-c2cccc3ccccc23)c1]

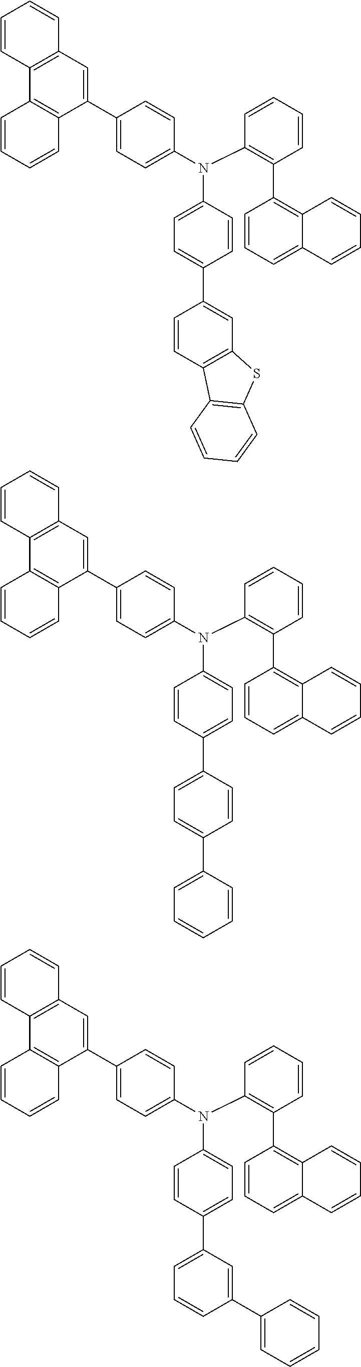 [SMILES: c1ccc(N(c2ccc(-c3ccc4c(c3)sc3ccccc34)cc2)c2ccc(-c3cc4ccccc4c4ccccc34)cc2)c(-c2cccc3ccccc23)c1 ; c1ccc(-c2ccc(-c3ccc(N(c4ccc(-c5cc6ccccc6c6ccccc56)cc4)c4ccccc4-c4cccc5ccccc45)cc3)cc2)cc1 ; c1ccc(-c2cccc(-c3ccc(N(c4ccc(-c5cc6ccccc6c6ccccc56)cc4)c4ccccc4-c4cccc5ccccc45)cc3)c2)cc1]

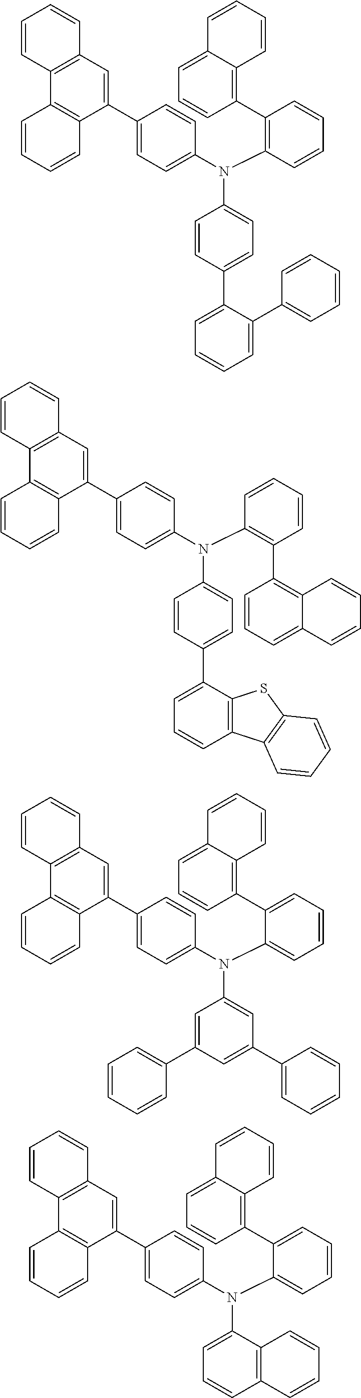

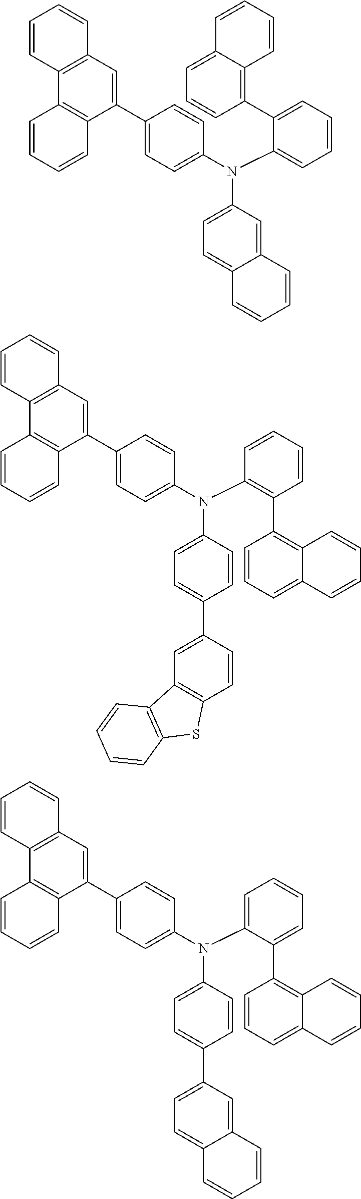 [SMILES: c1ccc(N(c2ccc(-c3cc4ccccc4c4ccccc34)cc2)c2ccc3ccccc3c2)c(-c2cccc3ccccc23)c1 ; c1ccc(N(c2ccc(-c3ccc4sc5ccccc5c4c3)cc2)c2ccc(-c3cc4ccccc4c4ccccc34)cc2)c(-c2cccc3ccccc23)c1 ; c1ccc(N(c2ccc(-c3ccc4ccccc4c3)cc2)c2ccc(-c3cc4ccccc4c4ccccc34)cc2)c(-c2cccc3ccccc23)c1]

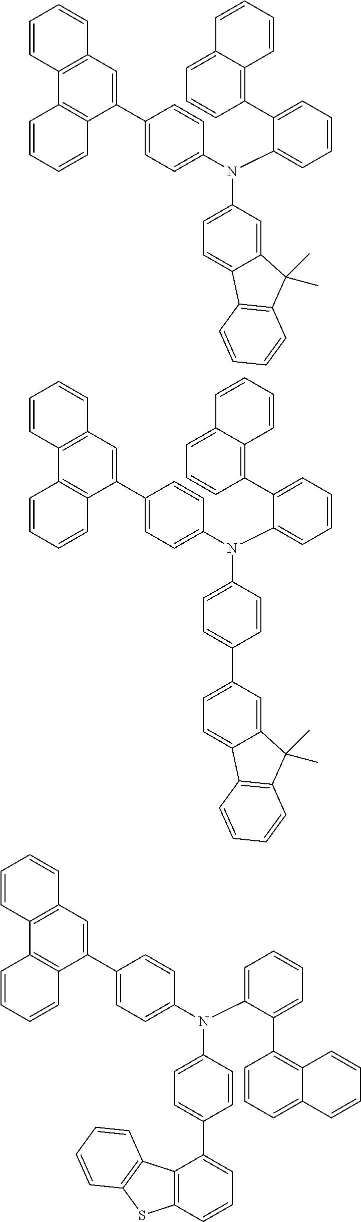

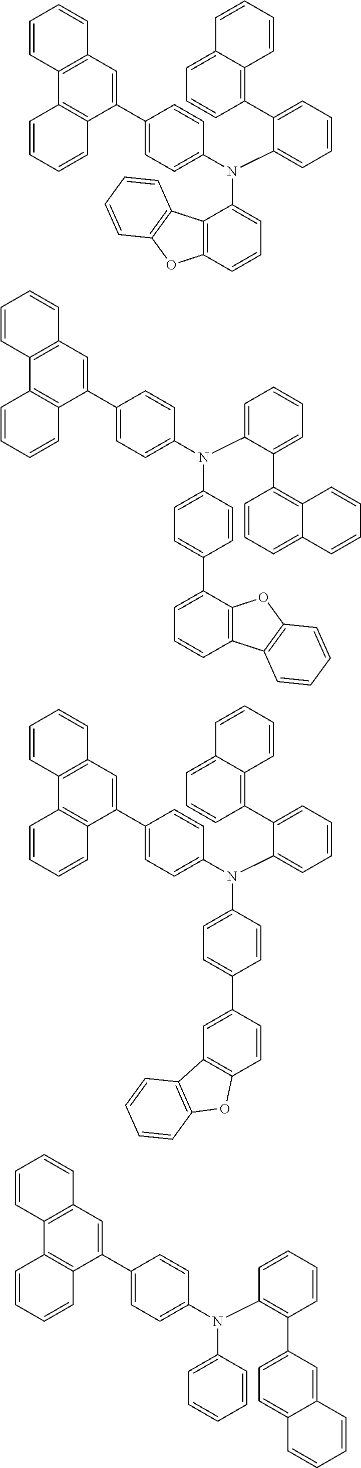

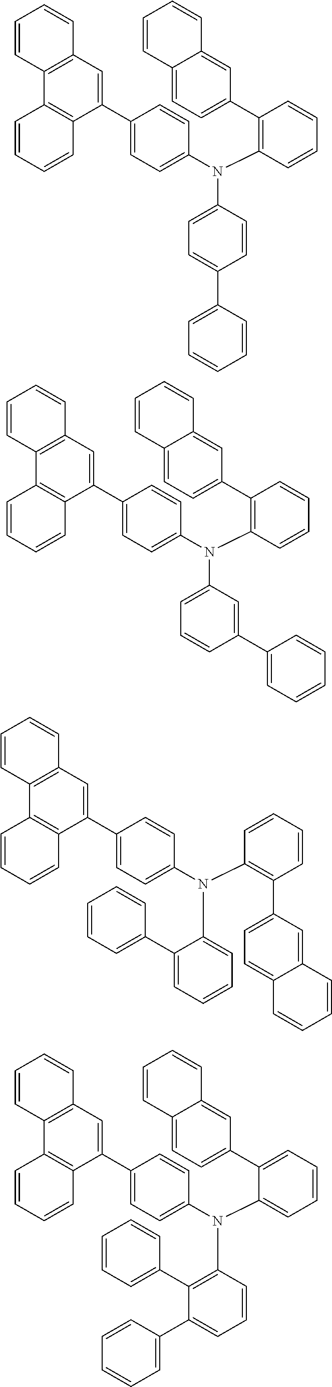

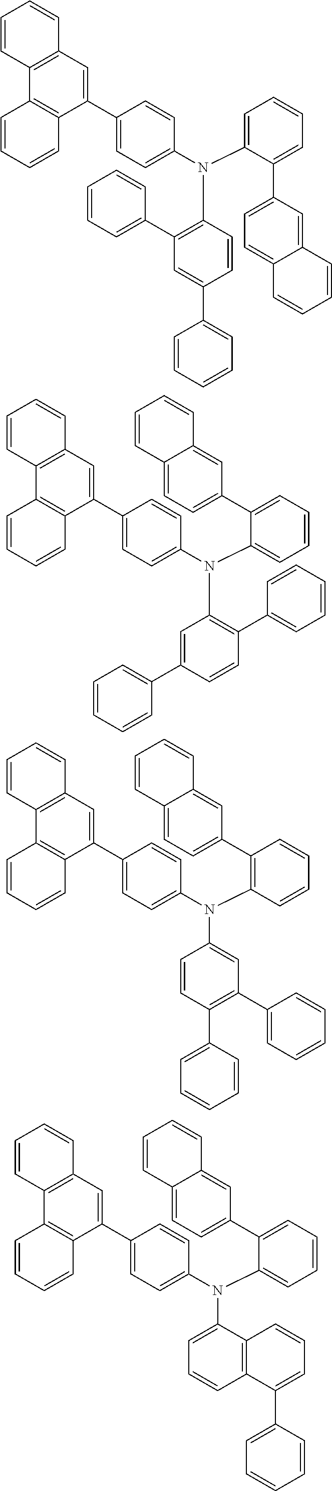

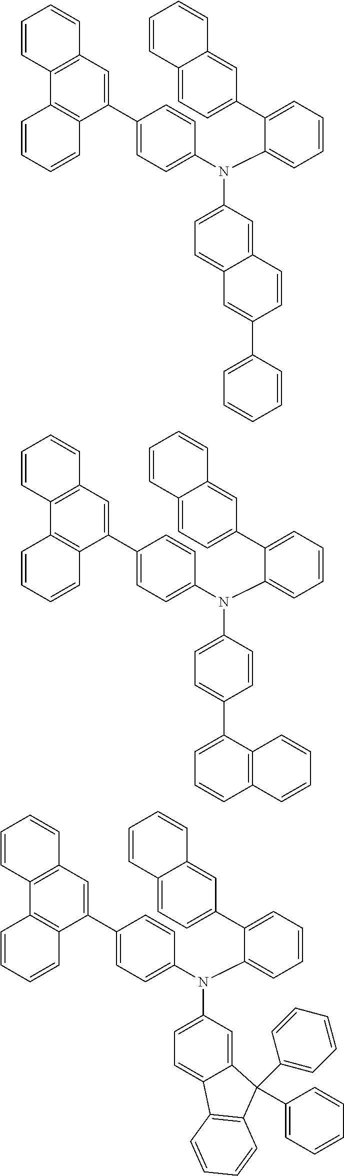

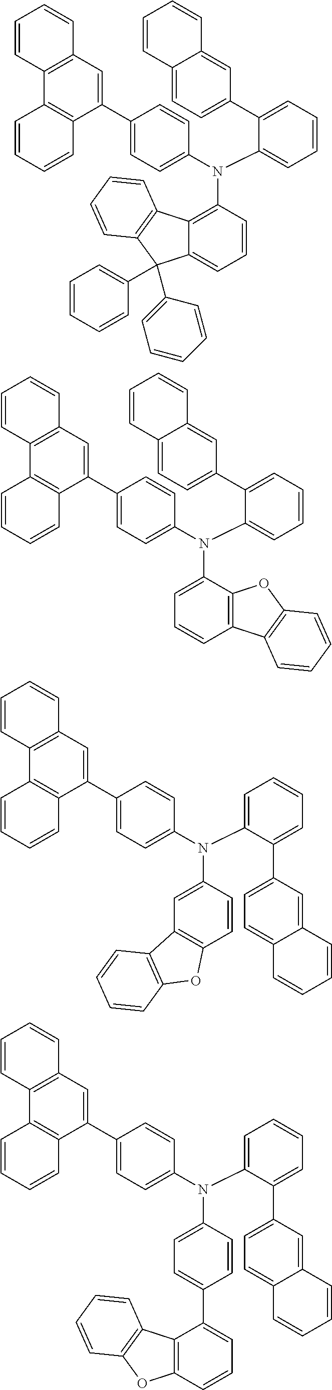

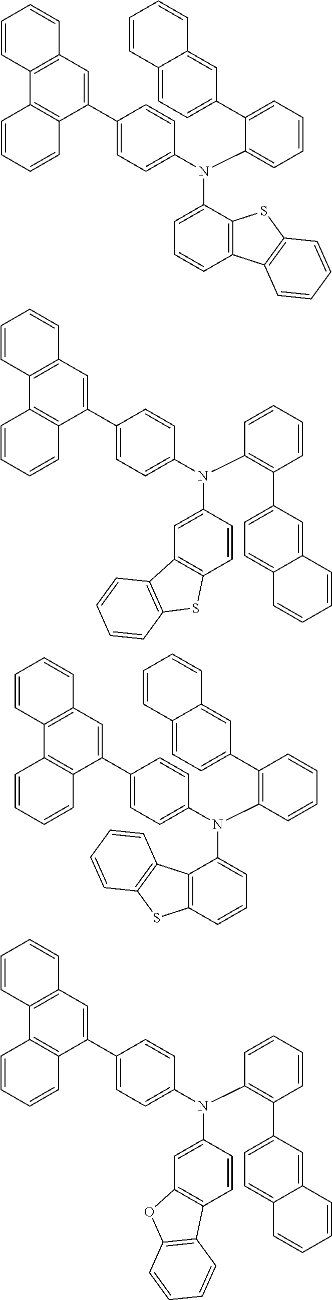

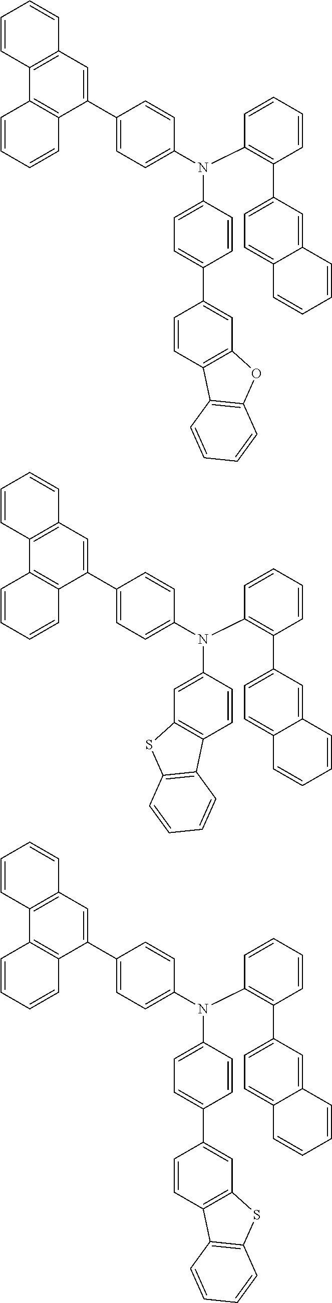 [SMILES: c1ccc(N(c2ccc(-c3ccc4c(c3)oc3ccccc34)cc2)c2ccc(-c3cc4ccccc4c4ccccc34)cc2)c(-c2ccc3ccccc3c2)c1 ; c1ccc(N(c2ccc(-c3cc4ccccc4c4ccccc34)cc2)c2ccc3c(c2)sc2ccccc23)c(-c2ccc3ccccc3c2)c1 ; c1ccc(N(c2ccc(-c3ccc4c(c3)sc3ccccc34)cc2)c2ccc(-c3cc4ccccc4c4ccccc34)cc2)c(-c2ccc3ccccc3c2)c1]

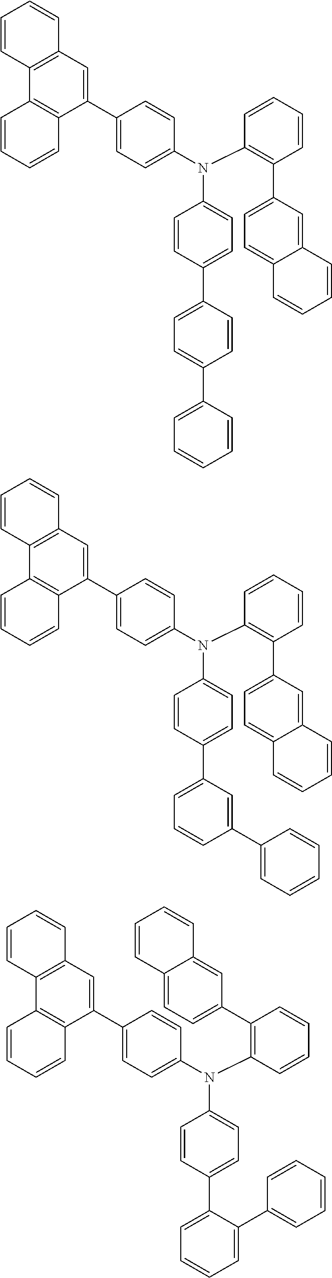 [SMILES: c1ccc(-c2ccc(-c3ccc(N(c4ccc(-c5cc6ccccc6c6ccccc56)cc4)c4ccccc4-c4ccc5ccccc5c4)cc3)cc2)cc1 ; c1ccc(-c2cccc(-c3ccc(N(c4ccc(-c5cc6ccccc6c6ccccc56)cc4)c4ccccc4-c4ccc5ccccc5c4)cc3)c2)cc1 ; c1ccc(-c2ccccc2-c2ccc(N(c3ccc(-c4cc5ccccc5c5ccccc45)cc3)c3ccccc3-c3ccc4ccccc4c3)cc2)cc1]

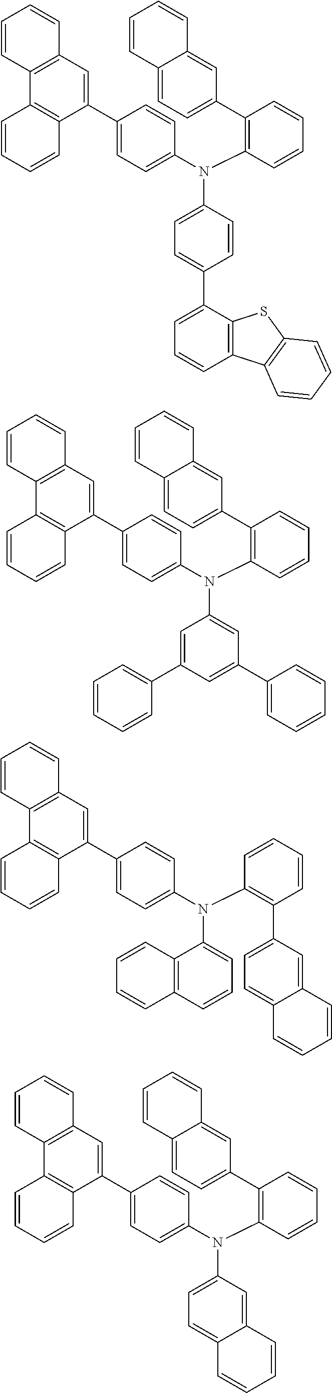 [SMILES: c1ccc(N(c2ccc(-c3cc4ccccc4c4ccccc34)cc2)c2ccc(-c3cccc4c3sc3ccccc34)cc2)c(-c2ccc3ccccc3c2)c1 ; c1ccc(-c2cc(-c3ccccc3)cc(N(c3ccc(-c4cc5ccccc5c5ccccc45)cc3)c3ccccc3-c3ccc4ccccc4c3)c2)cc1 ; c1ccc(N(c2ccc(-c3cc4ccccc4c4ccccc34)cc2)c2cccc3ccccc23)c(-c2ccc3ccccc3c2)c1 ; c1ccc(N(c2ccc(-c3cc4ccccc4c4ccccc34)cc2)c2ccc3ccccc3c2)c(-c2ccc3ccccc3c2)c1]

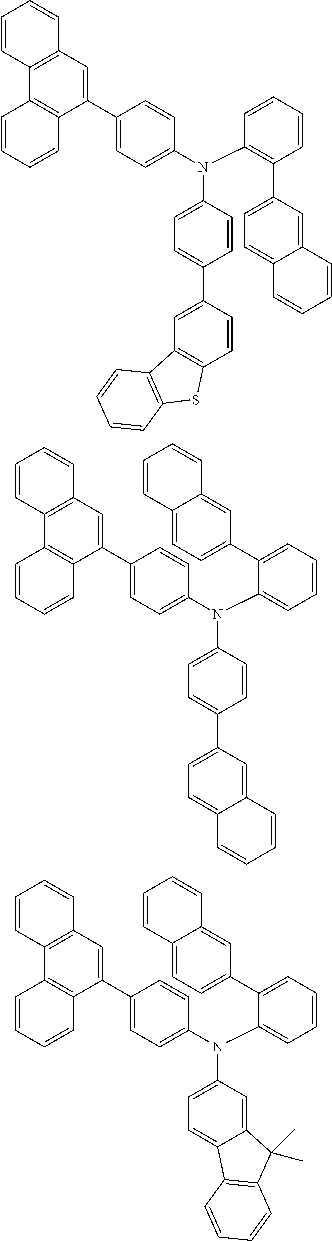

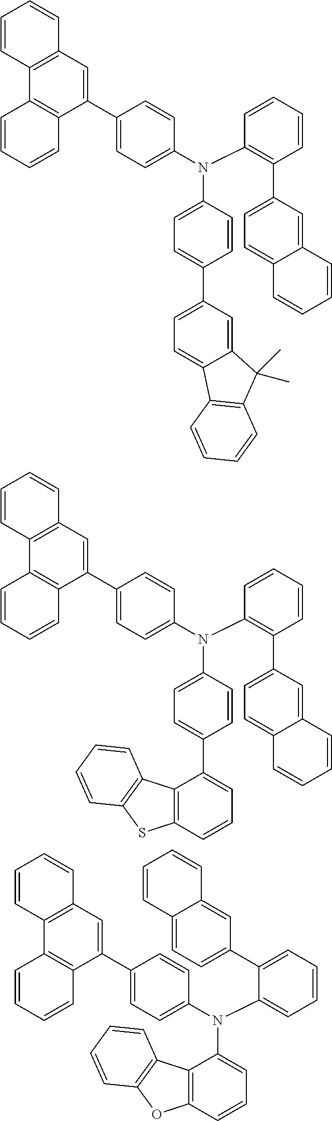

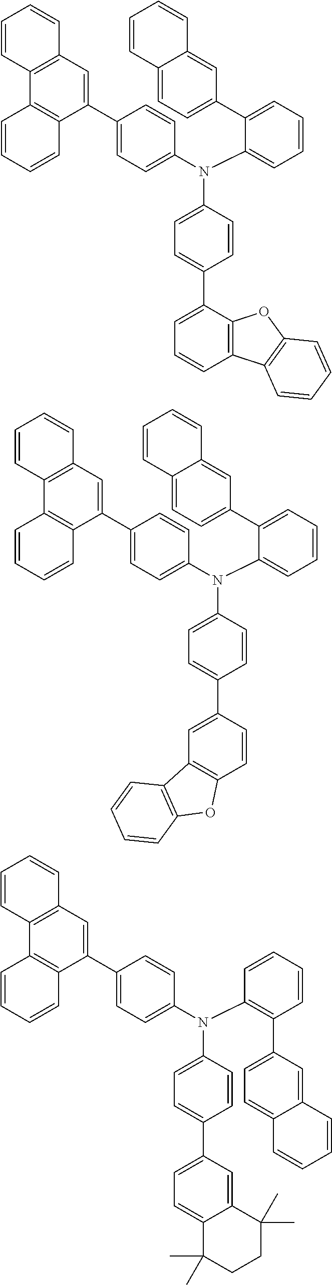 [SMILES: c1ccc(N(c2ccc(-c3cc4ccccc4c4ccccc34)cc2)c2ccc(-c3cccc4c3oc3ccccc34)cc2)c(-c2ccc3ccccc3c2)c1 ; c1ccc(N(c2ccc(-c3ccc4oc5ccccc5c4c3)cc2)c2ccc(-c3cc4ccccc4c4ccccc34)cc2)c(-c2ccc3ccccc3c2)c1 ; CC1(C)CCC(C)(C)c2cc(-c3ccc(N(c4ccc(-c5cc6ccccc6c6ccccc56)cc4)c4ccccc4-c4ccc5ccccc5c4)cc3)ccc21]

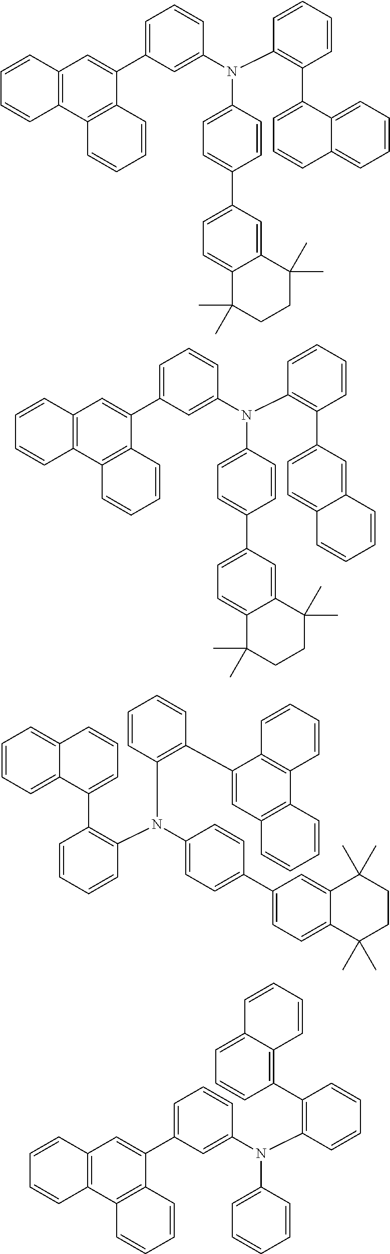

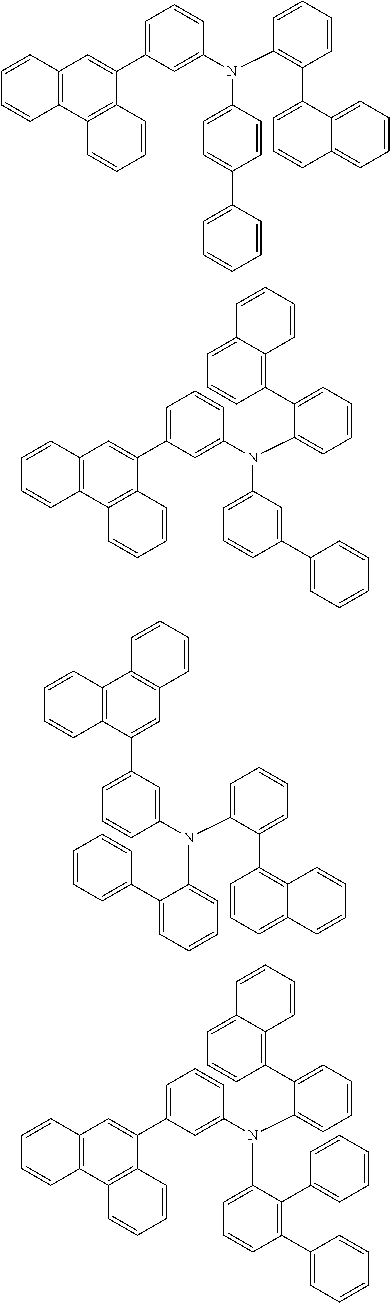

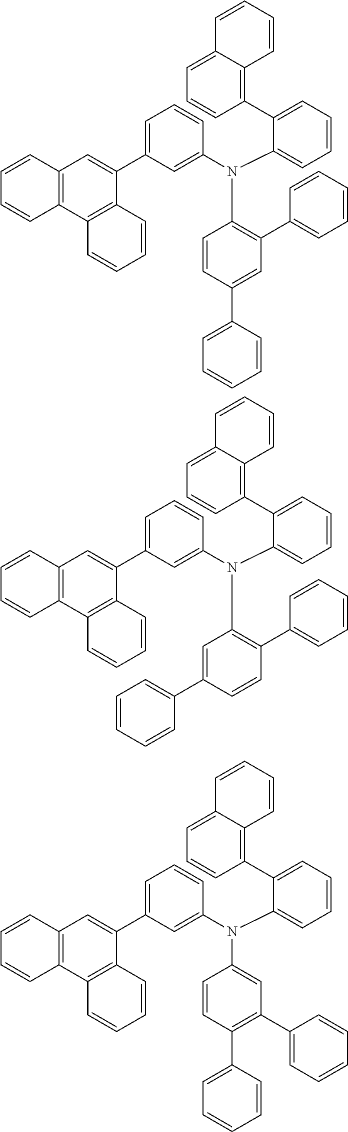

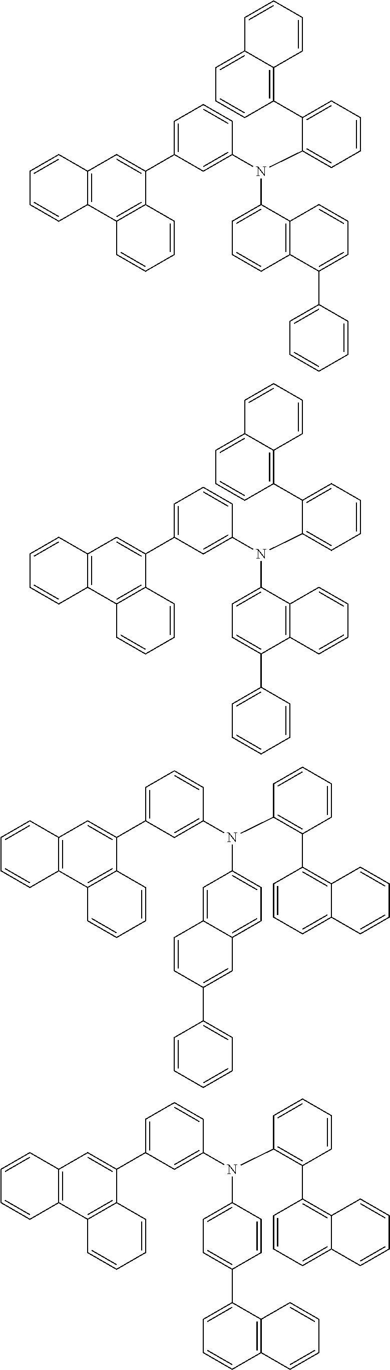

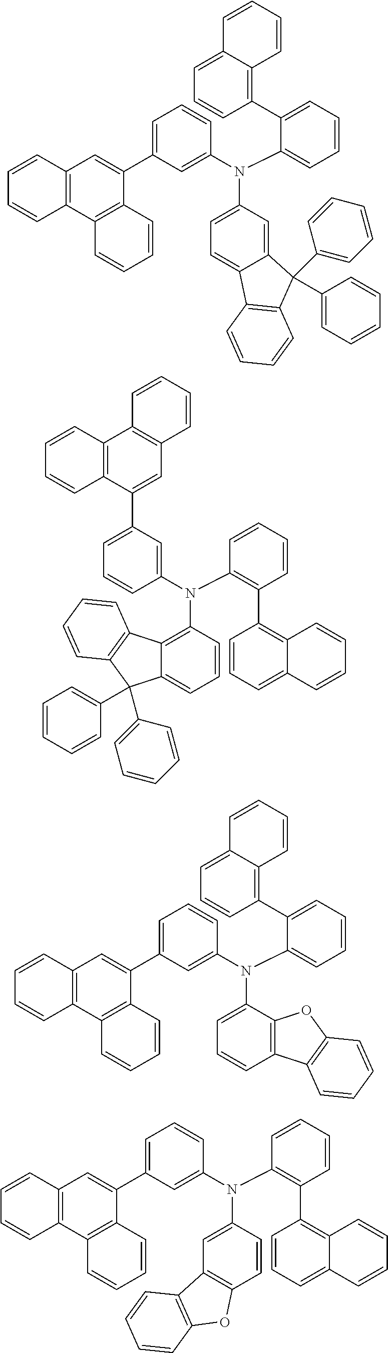

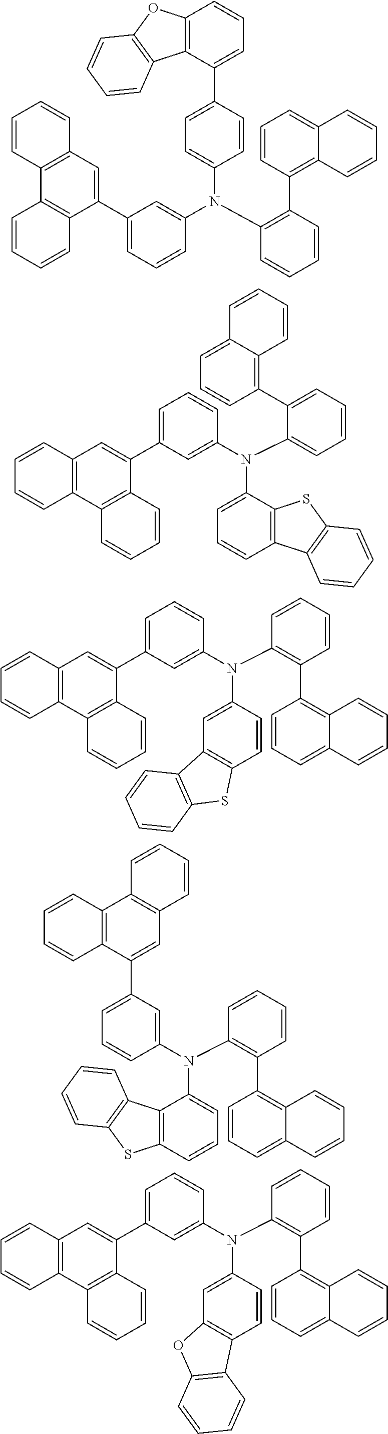

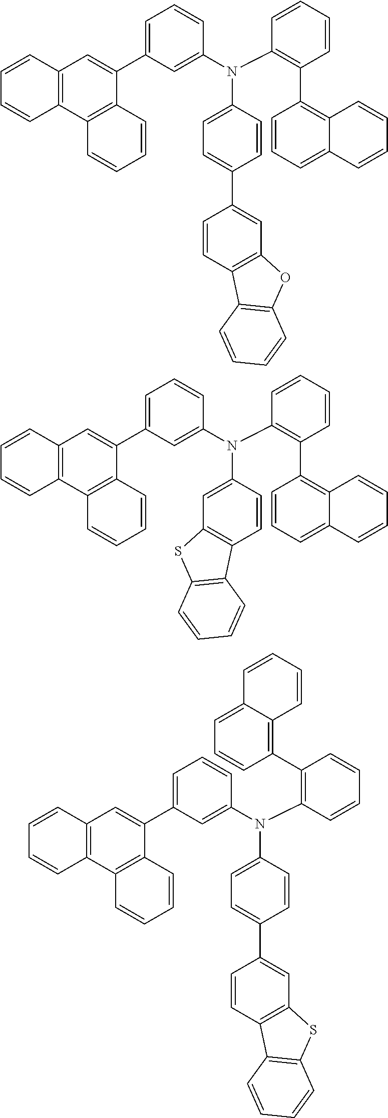

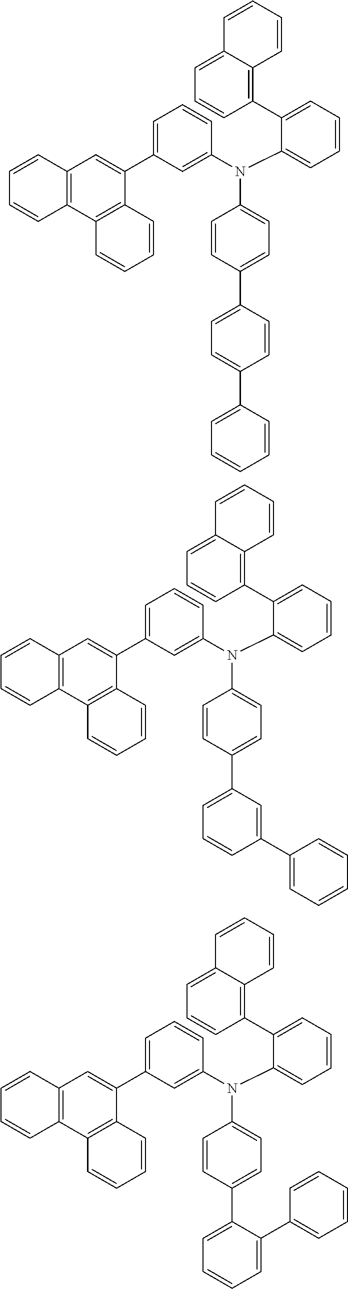

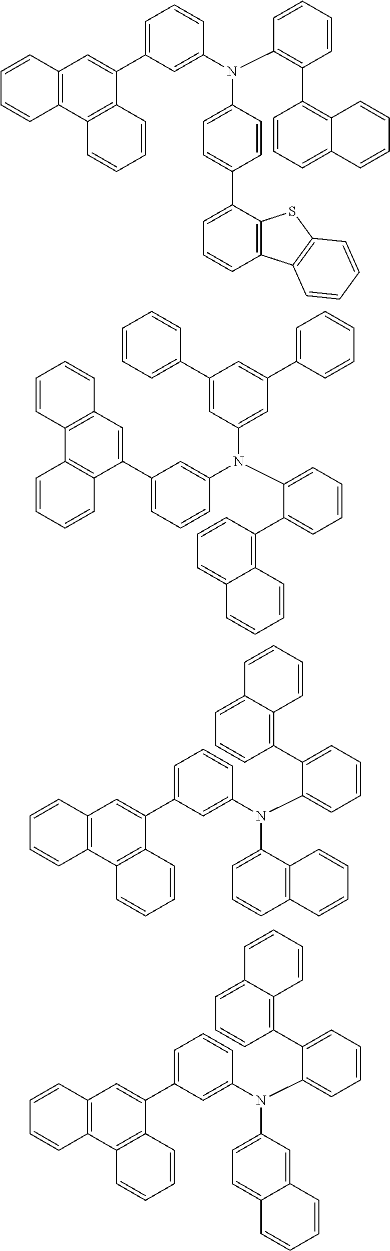

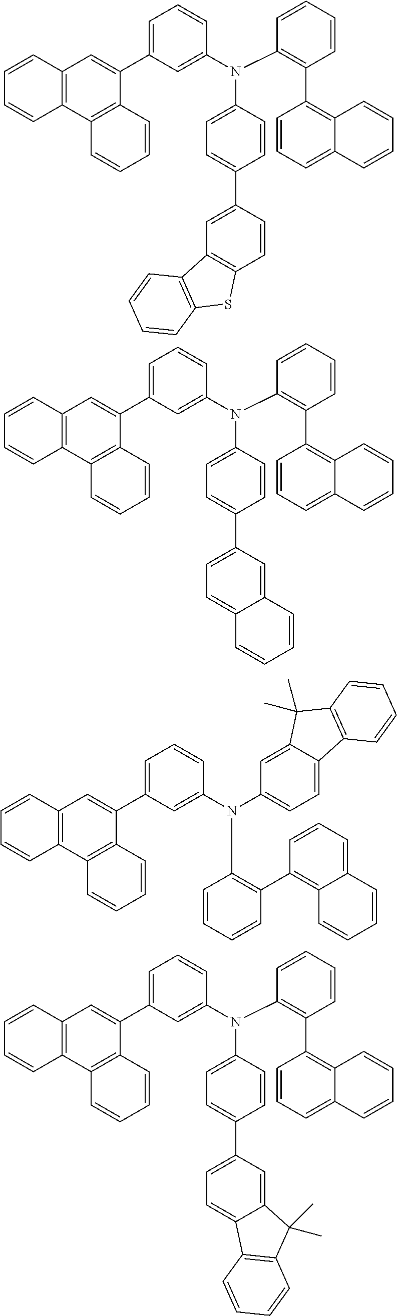

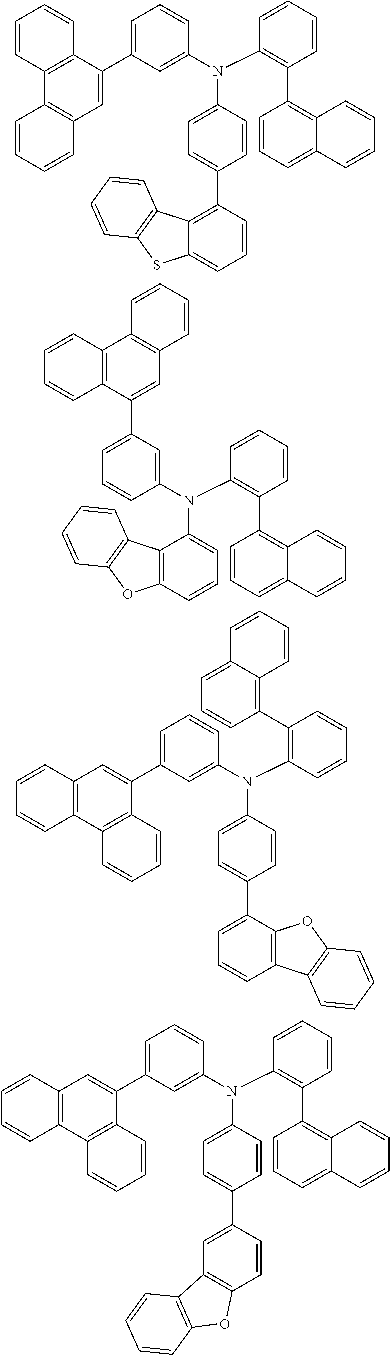

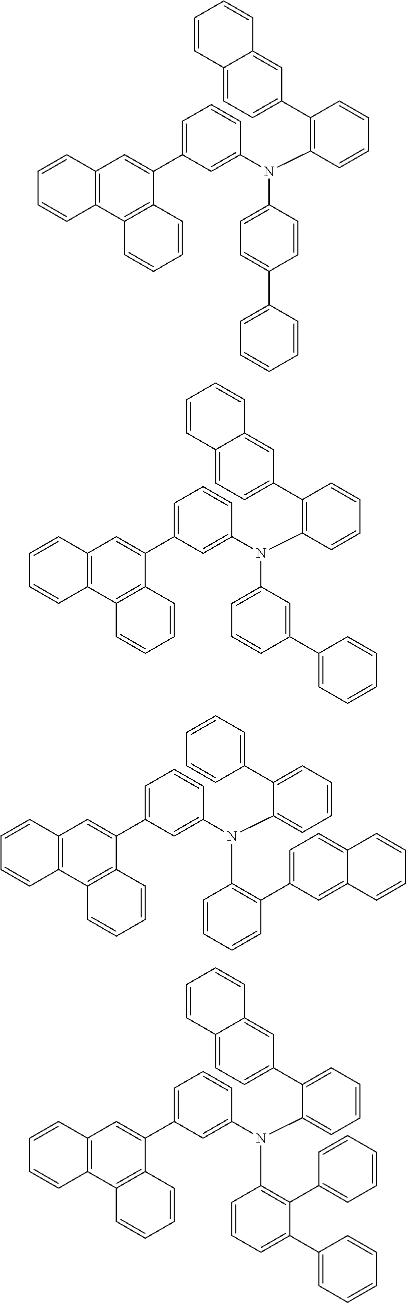

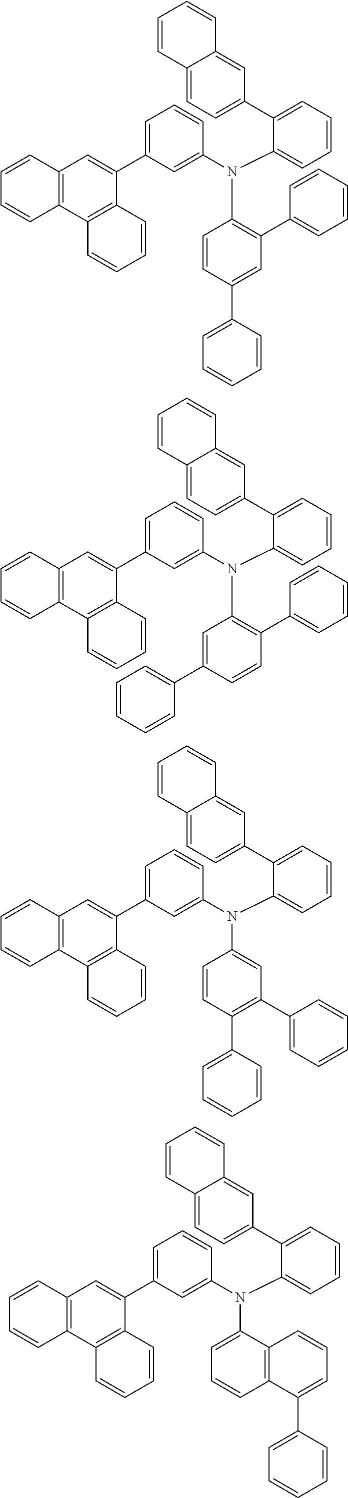

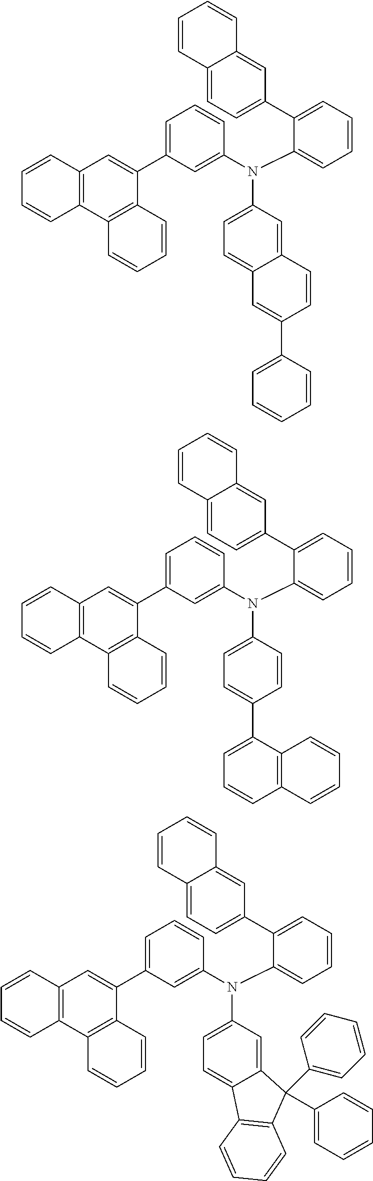 [SMILES: c1ccc(-c2ccc3cc(N(c4cccc(-c5cc6ccccc6c6ccccc56)c4)c4ccccc4-c4ccc5ccccc5c4)ccc3c2)cc1 ; c1cc(-c2cc3ccccc3c3ccccc23)cc(N(c2ccc(-c3cccc4ccccc34)cc2)c2ccccc2-c2ccc3ccccc3c2)c1 ; c1ccc(C2(c3ccccc3)c3ccccc3-c3ccc(N(c4cccc(-c5cc6ccccc6c6ccccc56)c4)c4ccccc4-c4ccc5ccccc5c4)cc32)cc1]

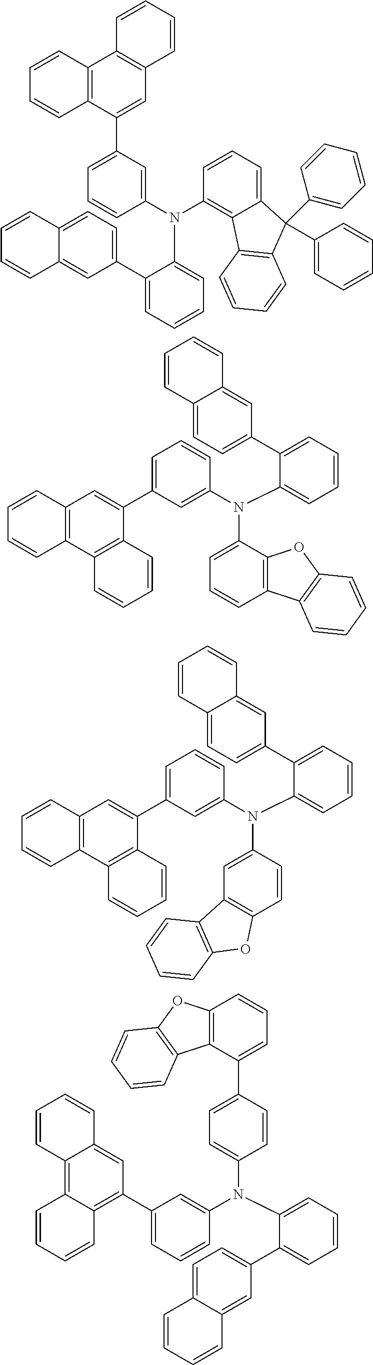

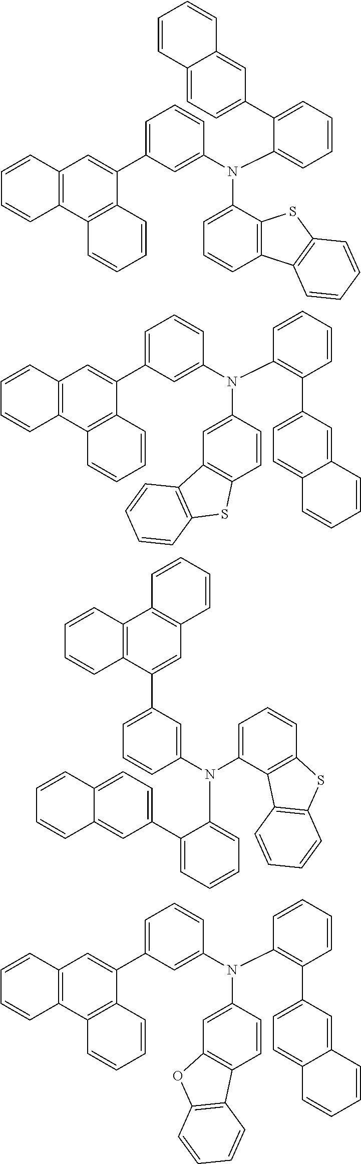

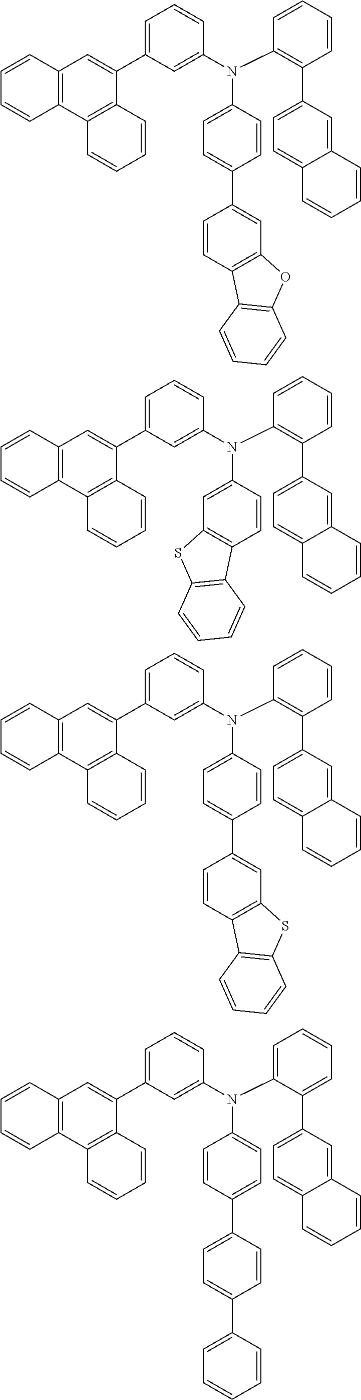

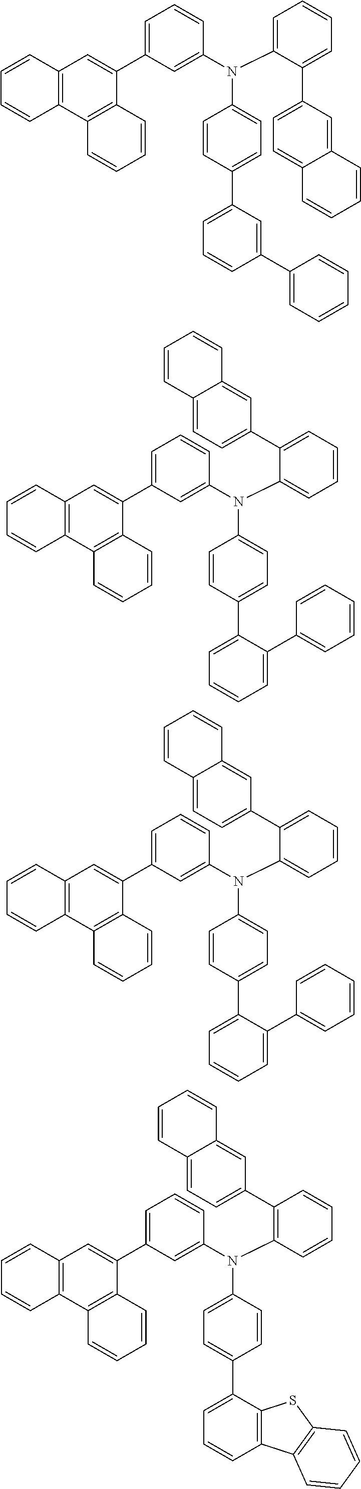

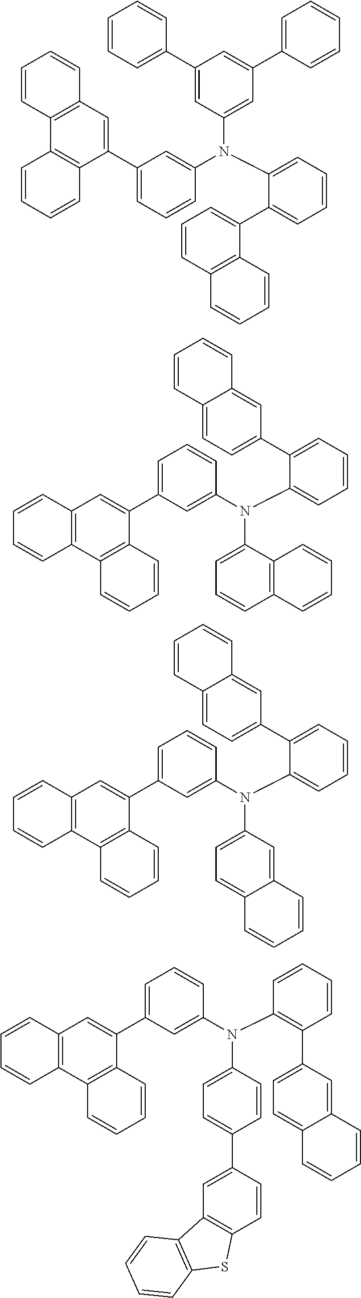

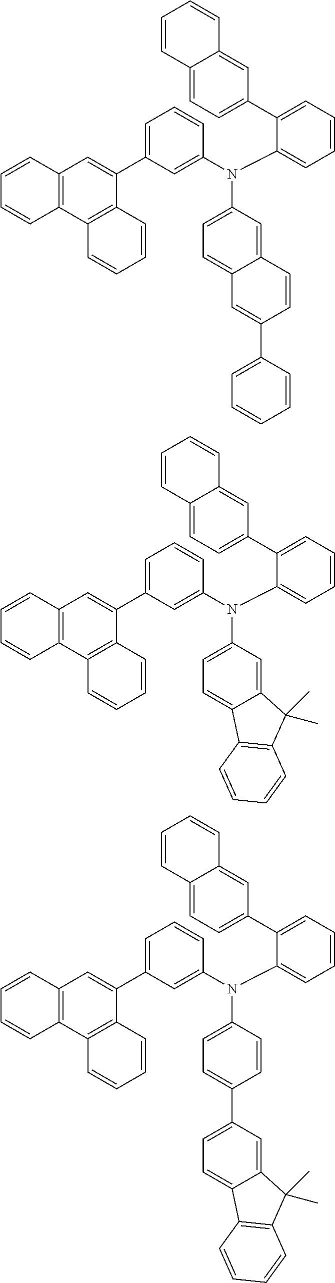

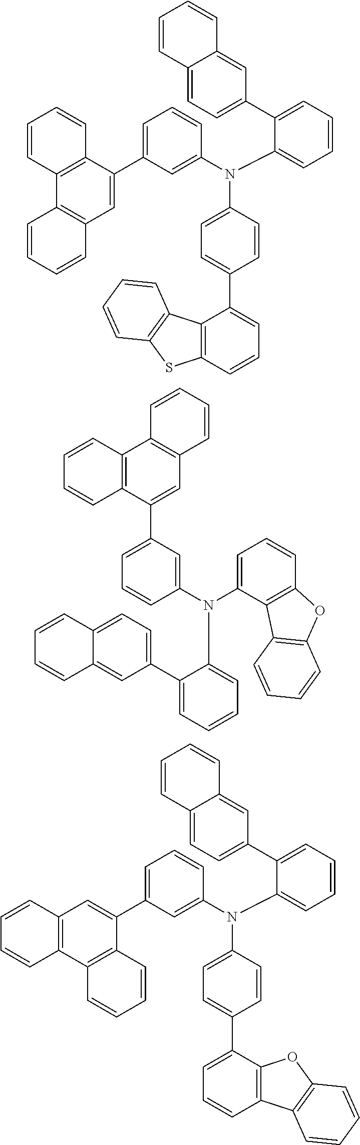

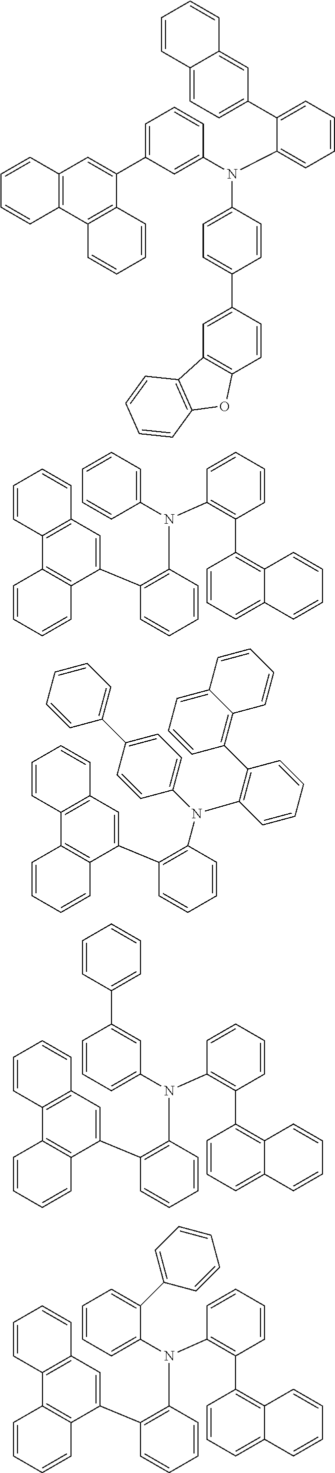

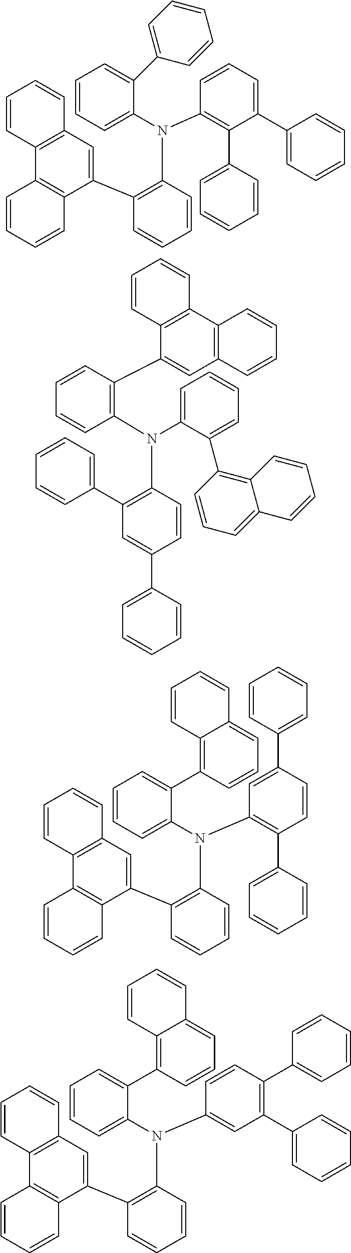

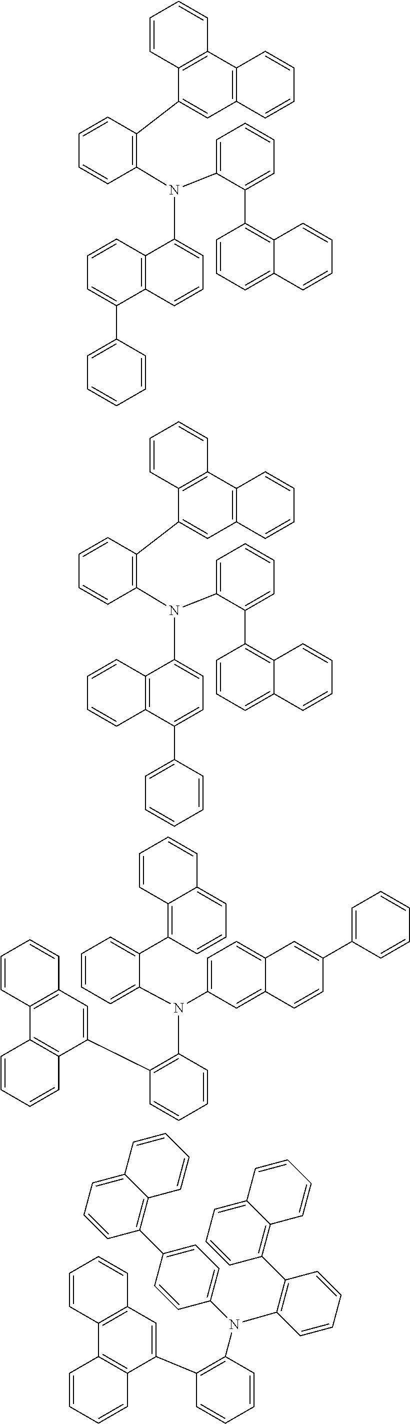

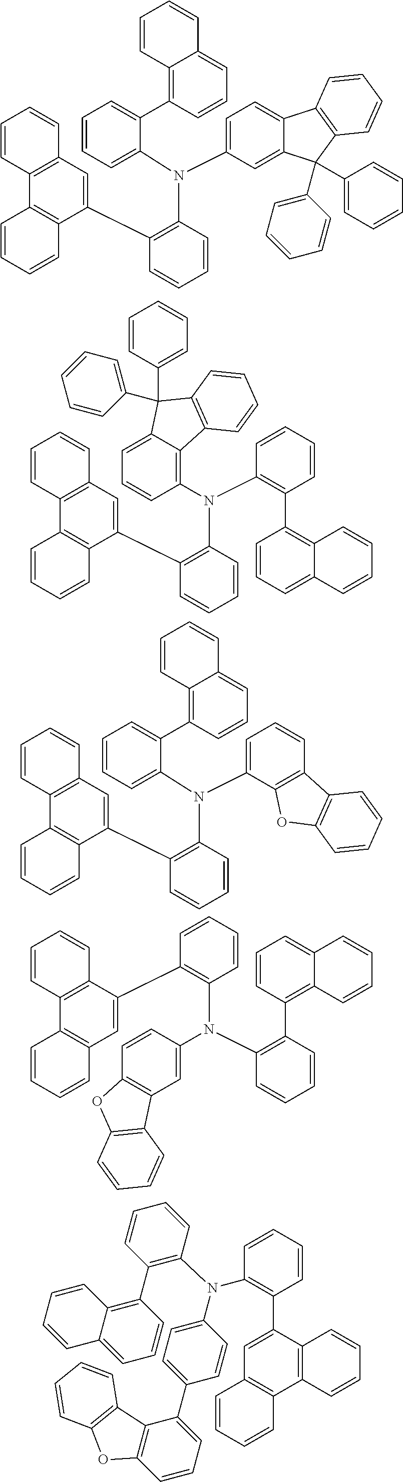

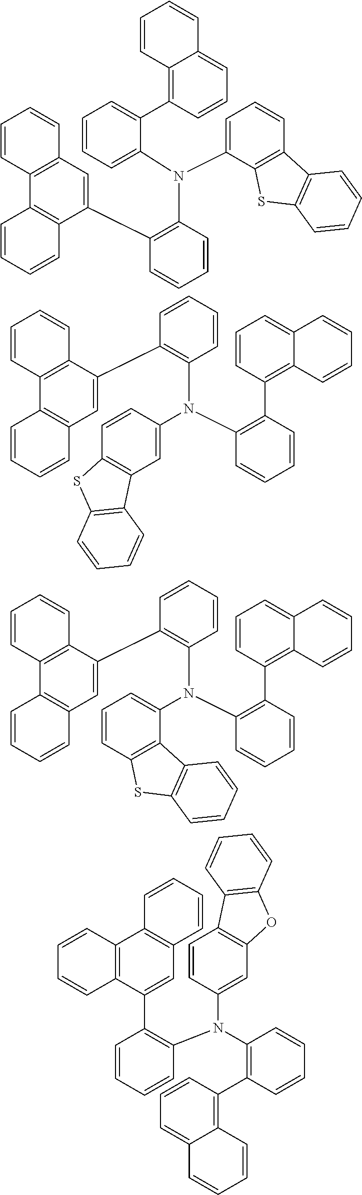

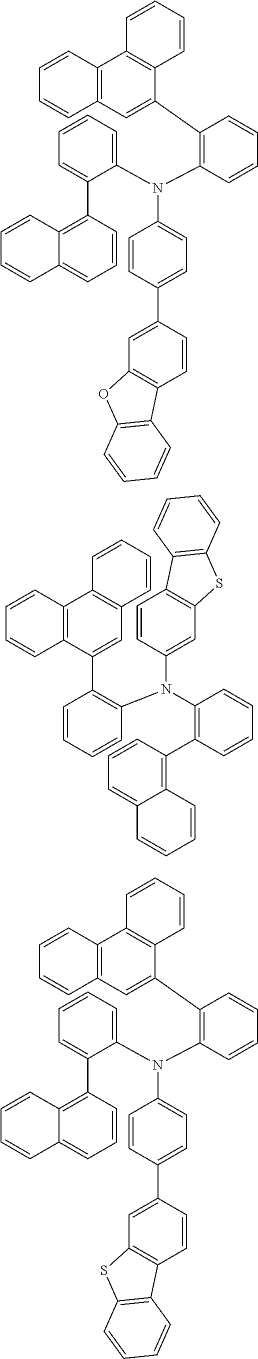

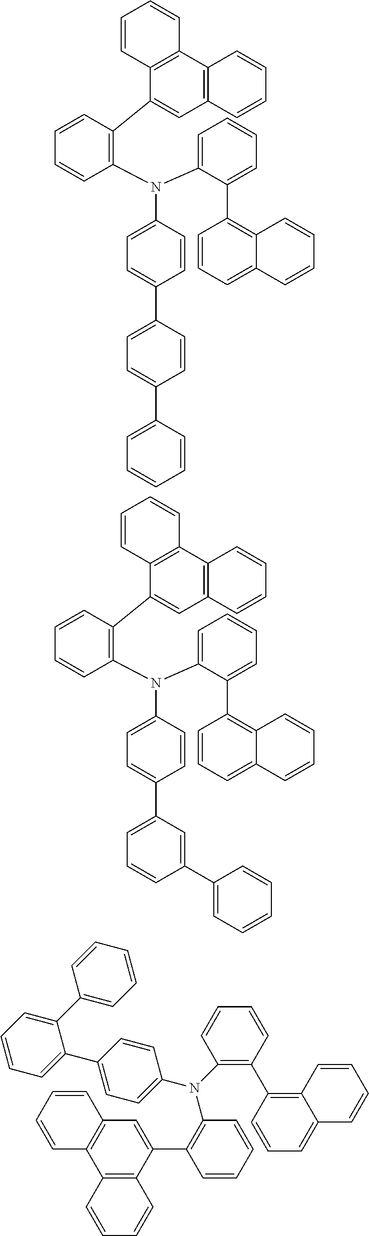

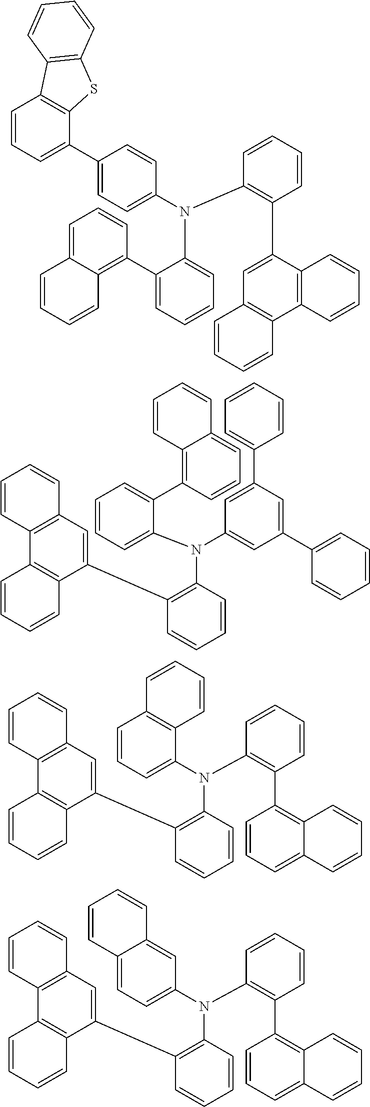

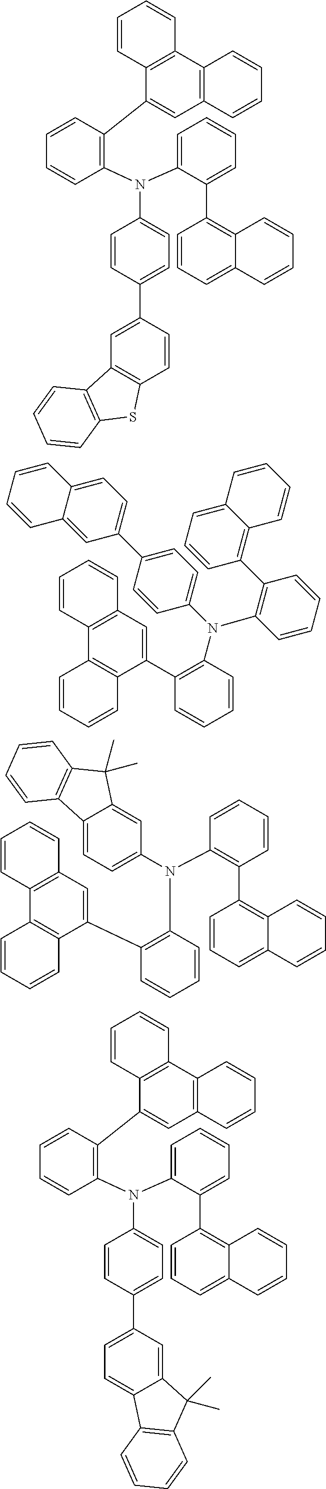

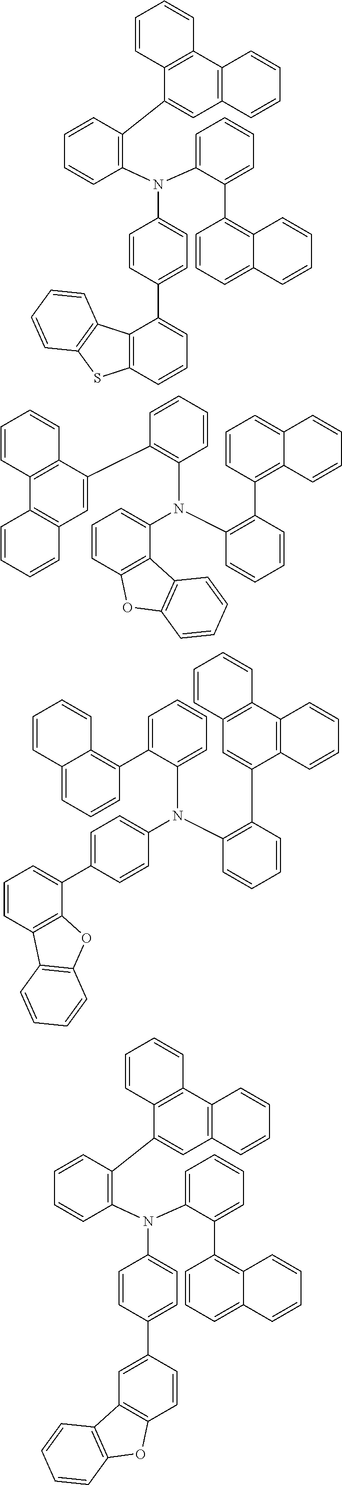

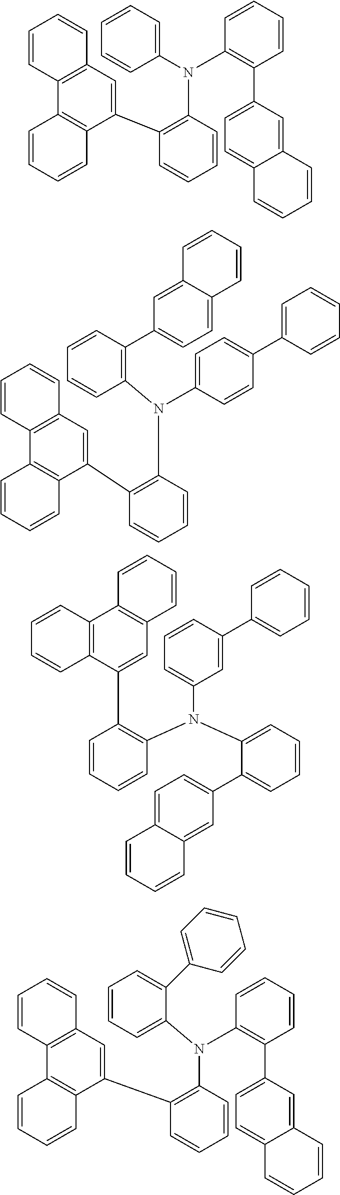

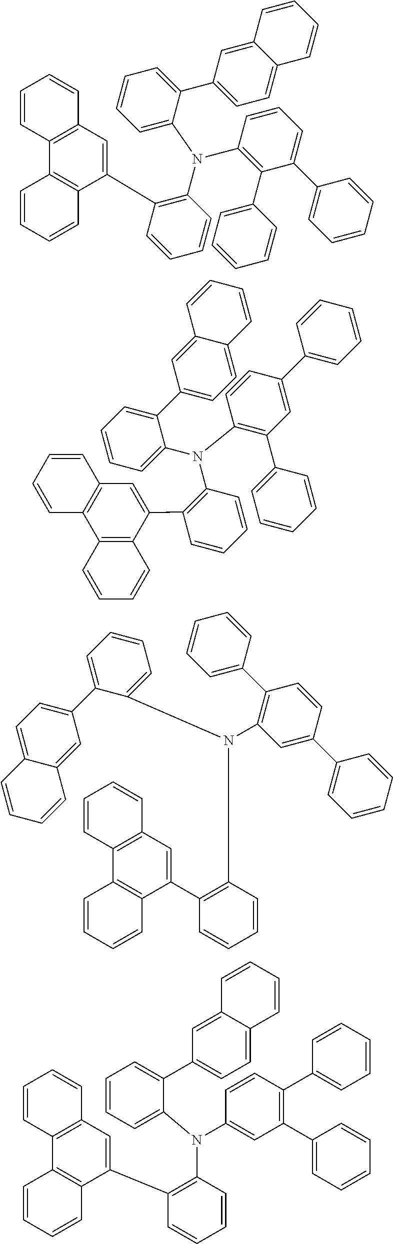

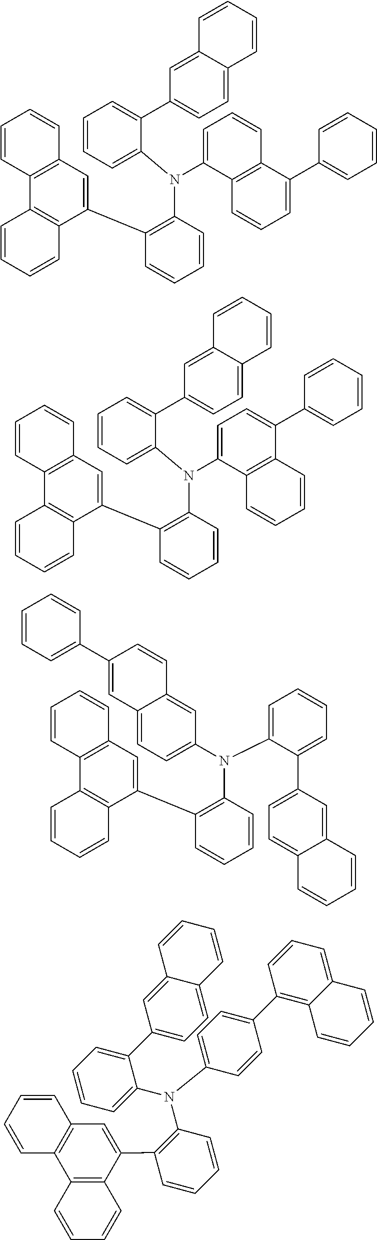

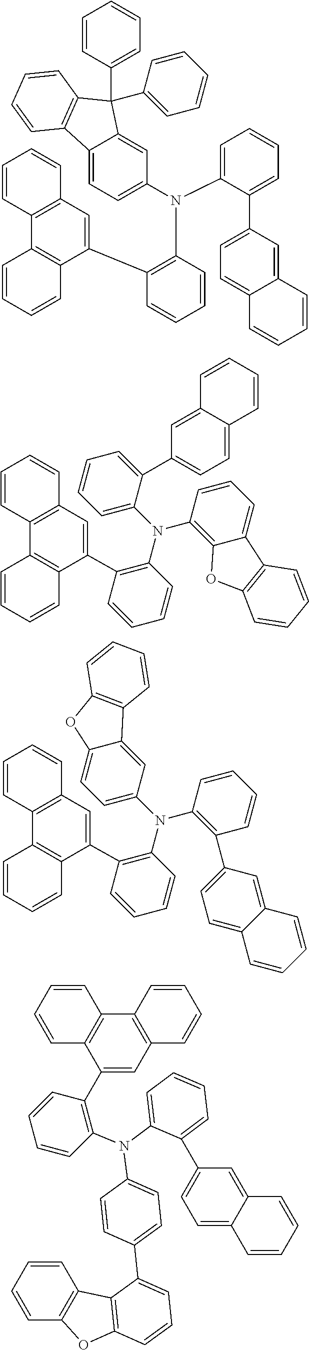

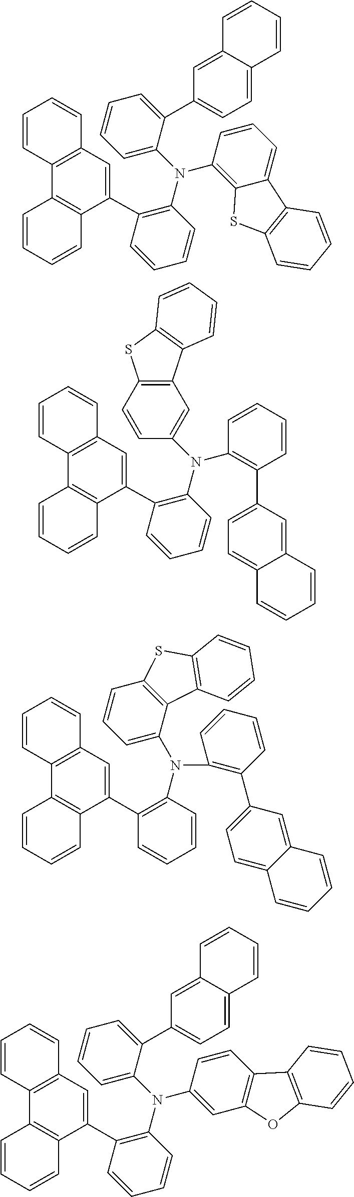

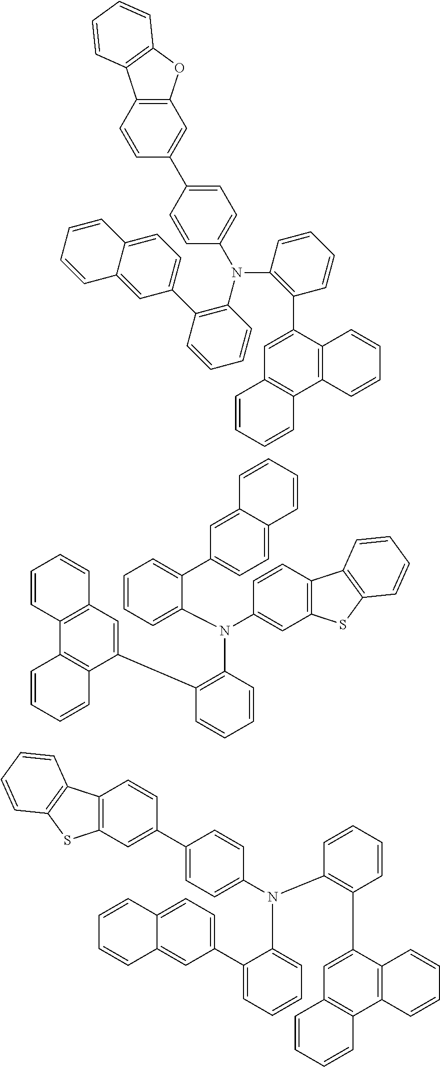

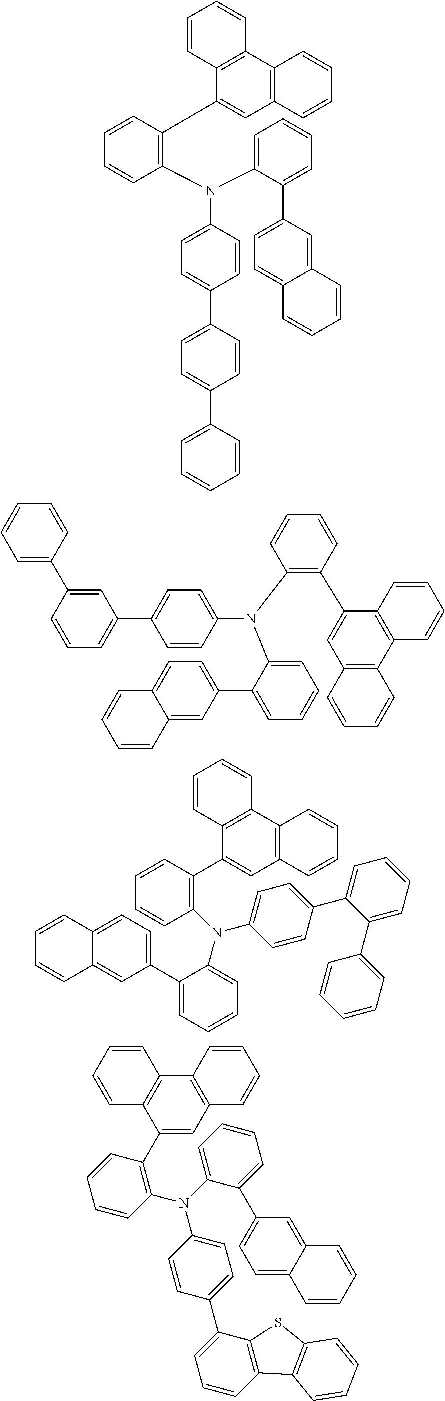

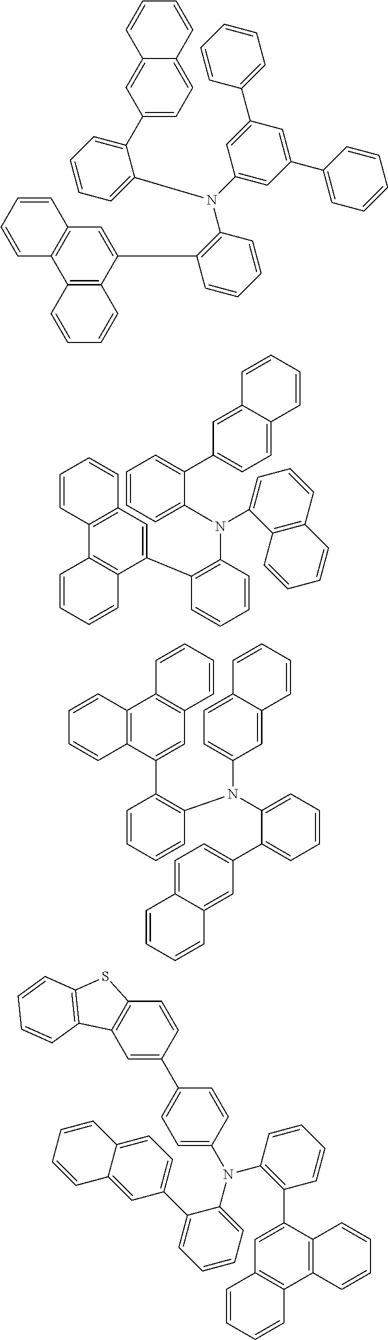

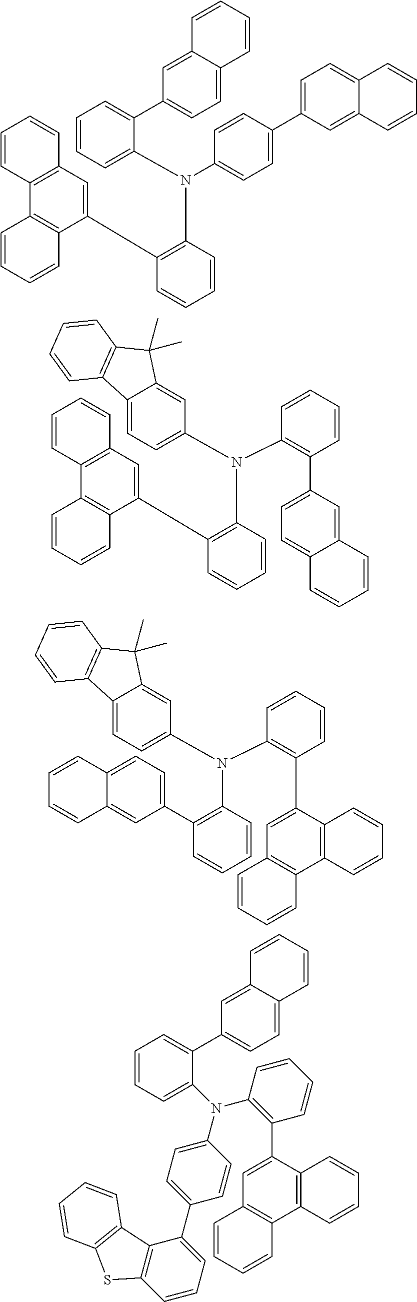

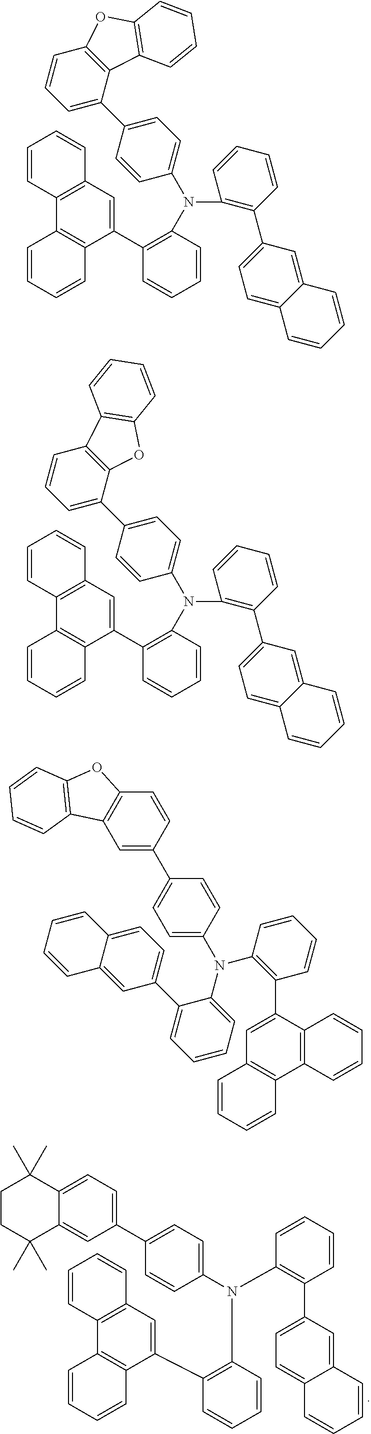 [SMILES: c1ccc(N(c2ccc(-c3cccc4oc5ccccc5c34)cc2)c2ccccc2-c2cc3ccccc3c3ccccc23)c(-c2ccc3ccccc3c2)c1 ; c1ccc(N(c2ccc(-c3cccc4c3oc3ccccc34)cc2)c2ccccc2-c2cc3ccccc3c3ccccc23)c(-c2ccc3ccccc3c2)c1 ; c1ccc(N(c2ccc(-c3ccc4oc5ccccc5c4c3)cc2)c2ccccc2-c2cc3ccccc3c3ccccc23)c(-c2ccc3ccccc3c2)c1 ; CC1(C)CCC(C)(C)c2cc(-c3ccc(N(c4ccccc4-c4ccc5ccccc5c4)c4ccccc4-c4cc5ccccc5c5ccccc45)cc3)ccc21]

The substituents of the compound of Chemical Formula 1 and the compound of Chemical Formula 2 can be bonded by a method known in the art, and the type and position of the substituent or the number of substituents can be changed according to the technology known in the art. For example, the substituents of the compound of Chemical Formula 1 and the compound of Chemical Formula 2 can be synthesized by the following Reaction Schemes 1 and 2.

For example, the compound of Chemical Formula 1 can be prepared as in the following Reaction Scheme 1. The substituents can be bonded by a method known in the art, and the type or position of the substituent or the number of substituents can be changed according to the technology known in the art.

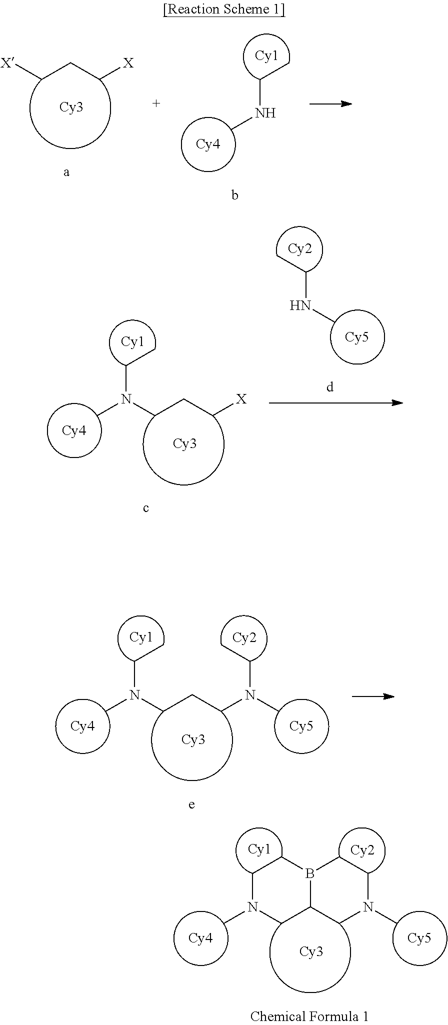

In Reaction Scheme 1, cy1 to cy5 are the same as the definitions of Chemical Formula 1, and X and X′ are different from each other, and can be each independently a halogen group of C1, Br, I, and the like.

After Compound a, Compound b, sodium-tert-butoxide, and 1.0 g of bis(tri-tert-butylphosphine)-palladium(0) were put into toluene under a nitrogen atmosphere, the resulting mixture was stirred under reflux for a certain period of time. After the completion of the reaction, the resulting product was extracted, and then recrystallized to obtain Compound c.

After Compound c, Compound d, sodium-tert-butoxide, and bis(tri-tert-butylphosphine)palladium(0) were put into toluene under a nitrogen atmosphere, the resulting mixture was stirred under reflux for a certain period of time. After the completion of the reaction, the resulting product was extracted, and then recrystallized to obtain Compound e.

After Compound e and boron triiodide were put into 1,2-dichlorobenzene under a nitrogen atmosphere, the resulting mixture was heated and stirred for a certain period of time. After the completion of the reaction, the resulting product was extracted, and then recrystallized to obtain a compound of Chemical Formula 1.

The compound of Chemical Formula 2 according to an exemplary embodiment of the present application can be prepared by a preparation method to be described below.

For example, the compound of Chemical Formula 2 can be prepared as in the following Reaction Scheme 2. The substituents can be bonded by a method known in the art, and the type or position of the substituent or the number of substituents can be changed according to the technology known in the art.

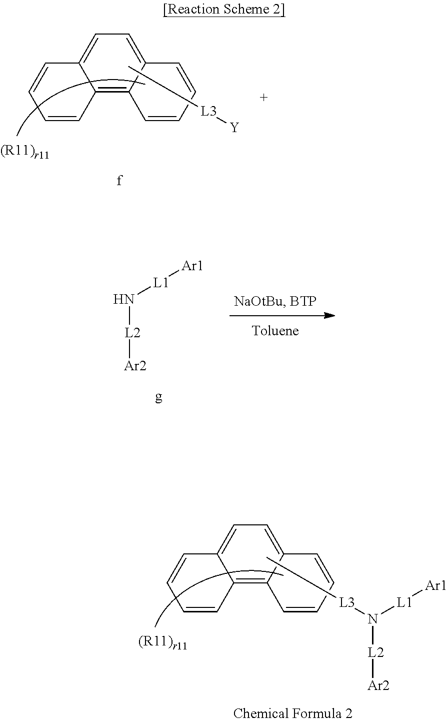

In Reaction Scheme 2, R11, r11, L1 to L3, Ar1, and Ar2 are the same as those defined in Chemical Formula 2, and Y can be a halogen group of C1, Br, I, and the like.

After toluene was added to Compound f, Compound g, and sodium tert-butoxide (NaOtBu), the resulting mixture was heated and stirred for a certain period of time. After bis(tri-tert-butylphosphine)palladium (BTP) dissolved in toluene was added to the mixture, the resulting mixture was heated and stirred for a certain period of time. After the completion of the reaction and filtration, the layers were separated with toluene and water. After the solvent was removed, the residue was recrystallized with ethyl acetate to obtain the compound of Chemical Formula 2.

A conjugation length and an energy band gap of the compound are closely associated with each other. Specifically, the longer a conjugation length of a compound is, the smaller an energy bandgap is.

In the present invention, various substituents can be introduced into the core structure as described above to synthesize compounds having various energy bandgaps. Further, in the present invention, various substituents can be introduced into the core structure having the structure described above to adjust the HOMO and LUMO energy levels of a compound.

In addition, various substituents can be introduced into the core structure having the structure described above to synthesize compounds having inherent characteristics of the introduced substituents. For example, a substituent usually used for a hole injection layer material, a material for transporting holes, a light emitting layer material, and an electron transport layer material, which are used for manufacturing an organic light emitting device, can be introduced into the core structure to synthesize a material which satisfies conditions required for each organic material layer.

Further, an organic light emitting device according to the present invention includes: an anode; a cathode; and an organic material layer provided between the anode and the cathode, in which the organic material layer includes a light emitting layer and a first organic material layer, the first organic material layer is provided between the anode and the light emitting layer, the light emitting layer includes the compound of Chemical Formula 1, and the first organic material layer includes the compound of Chemical Formula 2.

The organic light emitting device of the present invention can be manufactured using typical manufacturing methods and materials of an organic light emitting device, except that the above-described compound is used to form an organic material layer having one or more layers.

The compound can be formed as an organic material layer by not only a vacuum deposition method, but also a solution application method when an organic light emitting device is manufactured. Here, the solution application method means spin coating, dip coating, inkjet printing, screen printing, a spray method, roll coating, and the like, but is not limited thereto.

The organic material layer of the organic light emitting device of the present invention can be composed of a single-layered structure, but can be composed of a multi-layered structure in which two or more organic material layers are stacked. For example, the organic light emitting device of the present invention can have a structure including a hole injection layer, a hole transport layer, a layer which injects and transports holes simultaneously, a light emitting layer, an electron transport layer, an electron injection layer, and the like as organic material layers. However, the structure of the organic light emitting device is not limited thereto, and can include a fewer or greater number of organic material layers.

In an exemplary embodiment of the present specification, the first organic material layer is provided between the light emitting layer and the anode. That is, the first organic material layer is included in a hole transport region.

In an exemplary embodiment of the present specification, the first organic material is provided to be brought into direct contact with the light emitting layer. In this case, an additional organic material layer is not included between the light emitting layer and the first organic material layer.

In an exemplary embodiment of the present specification, the first organic material layer is provided between the light emitting layer and the anode, and provided to be brought into direct contact with the light emitting layer.

In an exemplary embodiment of the present specification, the first organic material layer is a hole injection layer, a hole transport layer, or an electron blocking layer.

In an exemplary embodiment of the present specification, the first organic material layer is an electron blocking layer.

In an exemplary embodiment of the present specification, the light emitting layer has a maximum light emission peak of 400 nm to 500 nm. That is, the light emitting layer is a blue light emitting layer.

In an exemplary embodiment of the present specification, the light emitting layer includes a host and a dopant, and the dopant includes the compound of Chemical Formula 1.

In an exemplary embodiment of the present specification, the light emitting layer includes the compound of Chemical Formula 1 as a dopant.

In an exemplary embodiment of the present specification, the light emitting layer includes the polycyclic compound of Chemical Formula 1 as a dopant, and can include a fluorescent host or a phosphorescent host.

In an exemplary embodiment of the present specification, the light emitting layer includes an anthracene-based compound as a host.

According to an exemplary embodiment of the present specification, the host includes a compound of the following Chemical Formula H:

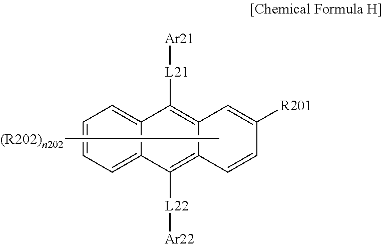

wherein in Chemical Formula H:

L21 and L22 are the same as or different from each other, and are each independently a direct bond, a substituted or unsubstituted arylene group, or a substituted or unsubstituted heteroarylene group;

Ar21 and Ar2 are the same as or different from each other, and are each independently a substituted or unsubstituted aryl group or a substituted or unsubstituted heterocyclic group;

R201 and R202 are the same as or different from each other, and are each independently hydrogen, deuterium, a halogen group, a substituted or unsubstituted alkyl group, a substituted or unsubstituted cycloalkyl group, a substituted or unsubstituted aryl group, or a substituted or unsubstituted heterocyclic group; and

n202 is an integer from 0 to 7, and when n202 is 2 or higher, the R202s are the same as or different from each other.

In an exemplary embodiment of the present specification, L21 and L22 are the same as or different from each other, and are each independently a direct bond, a monocyclic or polycyclic arylene group having 6 to 30 carbon atoms, or a monocyclic or polycyclic heteroarylene group having 2 to 30 carbon atoms.

In an exemplary embodiment of the present specification, L21 and L22 are the same as or different from each other, and are each independently a direct bond, a monocyclic or polycyclic arylene group having 6 to 20 carbon atoms, or a monocyclic or polycyclic heteroarylene group having 2 to 20 carbon atoms.

In an exemplary embodiment of the present specification, L21 and L22 are the same as or different from each other, and are each independently a direct bond; a phenylene group which is unsubstituted or substituted with deuterium; a biphenylylene group which is unsubstituted or substituted with deuterium; a naphthylene group which is unsubstituted or substituted with deuterium; a divalent dibenzofuran group; or a divalent dibenzothiophene group.

In an exemplary embodiment of the present specification, Ar21 and Ar22 are the same as or different from each other, and are each independently a substituted or unsubstituted monocyclic or polycyclic aryl group having 6 to 30 carbon atoms, or a substituted or unsubstituted monocyclic or polycyclic heterocyclic group having 2 to 30 carbon atoms.

In an exemplary embodiment of the present specification, Ar21 and Ar22 are the same as or different from each other, and are each independently a substituted or unsubstituted monocyclic or polycyclic aryl group having 6 to 20 carbon atoms, or a substituted or unsubstituted monocyclic or polycyclic heterocyclic group having 2 to 20 carbon atoms.

In an exemplary embodiment of the present specification, Ar21 and Ar22 are the same as or different from each other, and are each independently a substituted or unsubstituted monocyclic to tetracyclic aryl group having 6 to 20 carbon atoms, or a substituted or unsubstituted monocyclic to tetracyclic heterocyclic group having 6 to 20 carbon atoms.

In an exemplary embodiment of the present specification, Ar21 and Ar22 are the same as or different from each other, and are each independently a substituted or unsubstituted phenyl group, a substituted or unsubstituted biphenyl group, a substituted or unsubstituted terphenyl group, a substituted or unsubstituted naphthyl group, a substituted or unsubstituted anthracene group, a substituted or unsubstituted phenanthrene group, a substituted or unsubstituted phenalene group, a substituted or unsubstituted fluorene group, a substituted or unsubstituted benzofluorene group, a substituted or unsubstituted furan group, a substituted or unsubstituted thiophene group, a substituted or unsubstituted dibenzofuran group, a substituted or unsubstituted naphthobenzofuran group, a substituted or unsubstituted dibenzothiophene group, or a substituted or unsubstituted naphthobenzothiophene group.

In an exemplary embodiment of the present specification, Ar21 and Ar22 are the same as or different from each other, and are each independently a phenyl group which is unsubstituted or substituted with deuterium or a monocyclic or polycyclic aryl group having 6 to 20 carbon atoms; a biphenyl group which is unsubstituted or substituted with deuterium or a monocyclic or polycyclic aryl group having 6 to 20 carbon atoms; a naphthyl group which is unsubstituted or substituted with a monocyclic or polycyclic aryl group having 6 to 20 carbon groups; a dibenzofuran group which is unsubstituted or substituted with a monocyclic or polycyclic aryl group having 6 to 20 carbon atoms; a naphthobenzofuran group which is unsubstituted or substituted with a monocyclic or polycyclic aryl group having 6 to 20 carbon atoms; a dibenzothiophene group which is unsubstituted or substituted with a monocyclic or polycyclic aryl group having 6 to 20 carbon atoms; or a naphthobenzothiophene group which is unsubstituted or substituted with a monocyclic or polycyclic aryl group having 6 to 20 carbon atoms.

In an exemplary embodiment of the present specification, Ar21 and Ar22 are the same as or different from each other, and are each independently a phenyl group which is unsubstituted or substituted with deuterium; a biphenyl group which is unsubstituted or substituted with deuterium; a terphenyl group; a naphthyl group which is unsubstituted or substituted with deuterium; a phenanthrene group; a dibenzofuran group; a naphthobenzofuran group; a dibenzothiophene group; or a naphthobenzothiophene group.

In an exemplary embodiment of the present specification, any one of Ar21 and Ar22 is a substituted or unsubstituted aryl group, and the other is a substituted or unsubstituted heterocyclic group.

In an exemplary embodiment of the present specification, Ar21 is a substituted or unsubstituted aryl group, and Ar22 is a substituted or unsubstituted heterocyclic group.

In an exemplary embodiment of the present specification, Ar21 is a substituted or unsubstituted heterocyclic group, and Ar22 is a substituted or unsubstituted aryl group.

In an exemplary embodiment of the present specification, R201 is hydrogen; deuterium; a halogen group; a substituted or unsubstituted straight-chained or branched alkyl group having 1 to 30 carbon atoms; a substituted or unsubstituted monocyclic or polycyclic cycloalkyl group having 3 to 30 carbon atoms, a substituted or unsubstituted monocyclic or polycyclic aryl group having 6 to 30 carbon atoms, or a substituted or unsubstituted monocyclic or polycyclic heterocyclic group having 2 to 30 carbon atoms.

In an exemplary embodiment of the present specification, R201 is hydrogen, deuterium, fluorine, a substituted or unsubstituted straight-chained or branched alkyl group having 1 to 10 carbon atoms, a substituted or unsubstituted monocyclic or polycyclic cycloalkyl group having 3 to 10 carbon atoms, a substituted or unsubstituted monocyclic or polycyclic aryl group having 6 to 30 carbon atoms, or a substituted or unsubstituted monocyclic or polycyclic heterocyclic group having 2 to 30 carbon atoms.

In an exemplary embodiment of the present specification, R201 is hydrogen, a substituted or unsubstituted monocyclic or polycyclic aryl group having 6 to 30 carbon atoms, or a substituted or unsubstituted monocyclic or polycyclic heterocyclic group having 2 to 30 carbon atoms.

In an exemplary embodiment of the present specification, R201 is hydrogen, a substituted or unsubstituted monocyclic or polycyclic aryl group having 6 to 20 carbon atoms, or a substituted or unsubstituted monocyclic or polycyclic heterocyclic group having 2 to 20 carbon atoms.

In an exemplary embodiment of the present specification, R201 is hydrogen, a substituted or unsubstituted monocyclic to tetracyclic aryl group having 6 to 20 carbon atoms, or a substituted or unsubstituted monocyclic to tetracyclic heterocyclic group having 6 to 20 carbon atoms.

In an exemplary embodiment of the present specification, R201 is hydrogen, a substituted or unsubstituted phenyl group, a substituted or unsubstituted biphenyl group, a substituted or unsubstituted terphenyl group, a substituted or unsubstituted naphthyl group, a substituted or unsubstituted anthracene group, a substituted or unsubstituted phenanthrene group, a substituted or unsubstituted phenalene group, a substituted or unsubstituted fluorene group, a substituted or unsubstituted benzofluorene group, a substituted or unsubstituted furan group, a substituted or unsubstituted thiophene group, a substituted or unsubstituted dibenzofuran group, a substituted or unsubstituted naphthobenzofuran group, a substituted or unsubstituted dibenzothiophene group; or a substituted or unsubstituted naphthobenzothiophene group.

In an exemplary embodiment of the present specification, R201 is hydrogen; deuterium; a phenyl group which is unsubstituted or substituted with deuterium or a monocyclic or polycyclic aryl group having 6 to 20 carbon atoms; a biphenyl group which is unsubstituted or substituted with a monocyclic or polycyclic aryl group having 6 to 20 carbon atoms; a naphthyl group which is unsubstituted or substituted with deuterium or a monocyclic or polycyclic aryl group having 6 to 20 carbon atoms; a dibenzofuran group which is unsubstituted or substituted with a monocyclic or polycyclic aryl group having 6 to 20 carbon atoms; a naphthobenzofuran group which is unsubstituted or substituted with a monocyclic or polycyclic aryl group having 6 to 20 carbon atoms; a dibenzothiophene group which is unsubstituted or substituted with a monocyclic or polycyclic aryl group having 6 to 20 carbon atoms; or a naphthobenzothiophene group which is unsubstituted or substituted with a monocyclic or polycyclic aryl group having 6 to 20 carbon atoms.

In an exemplary embodiment of the present specification, R201 is hydrogen; deuterium; a phenyl group which is unsubstituted or substituted with deuterium, a phenyl group, or a naphthyl group; a biphenyl group; a naphthyl group which is unsubstituted or substituted with deuterium, a phenyl group, or a naphthyl group; a dibenzofuran group; a naphthobenzofuran group; a dibenzothiophene group; or a naphthobenzothiophene group.

According to an exemplary embodiment of the present specification, R202 is hydrogen or deuterium.

According to an exemplary embodiment of the present specification, four or more of the R202s are deuterium.

According to an exemplary embodiment of the present specification, R202 is hydrogen.

According to an exemplary embodiment of the present specification, R202 is deuterium.

In an exemplary embodiment of the present specification, when the compound of Chemical Formula H is substituted with deuterium, 30% or more of hydrogen at a substitutable position is substituted with deuterium. In another exemplary embodiment, in the structure of Chemical Formula H, 40% or more of hydrogen at a substitutable position is substituted with deuterium. In still another exemplary embodiment, in the structure of Chemical Formula H, 60% or more of hydrogen at a substitutable position is substituted with deuterium.

In yet another exemplary embodiment, in the structure of Chemical Formula H, 80% or more of hydrogen at a substitutable position is substituted with deuterium. In still yet another exemplary embodiment, in the structure of Chemical Formula H, 100% of hydrogen at a substitutable position is substituted with deuterium.

In an exemplary embodiment of the present specification, the compound of Chemical Formula H is any one compound selected from among the following compounds:

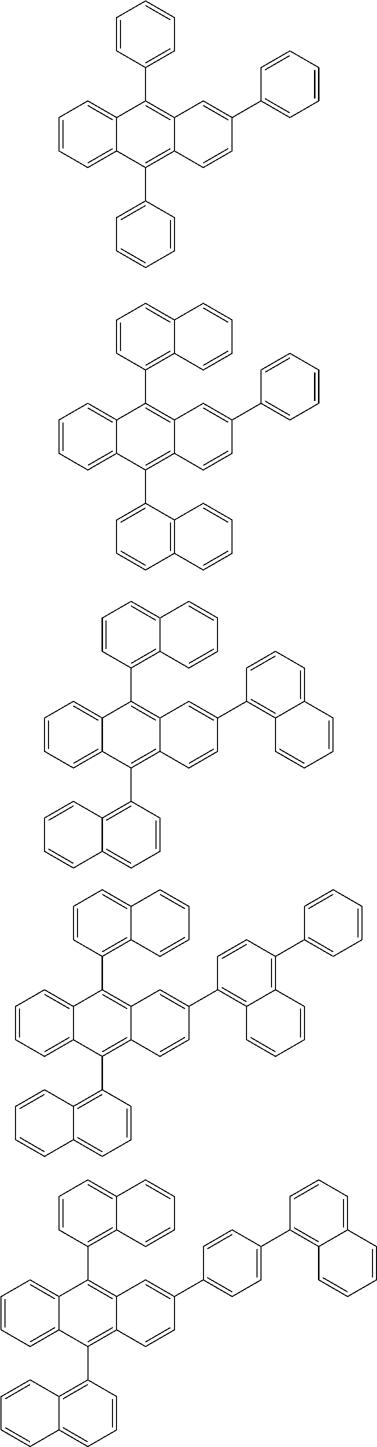

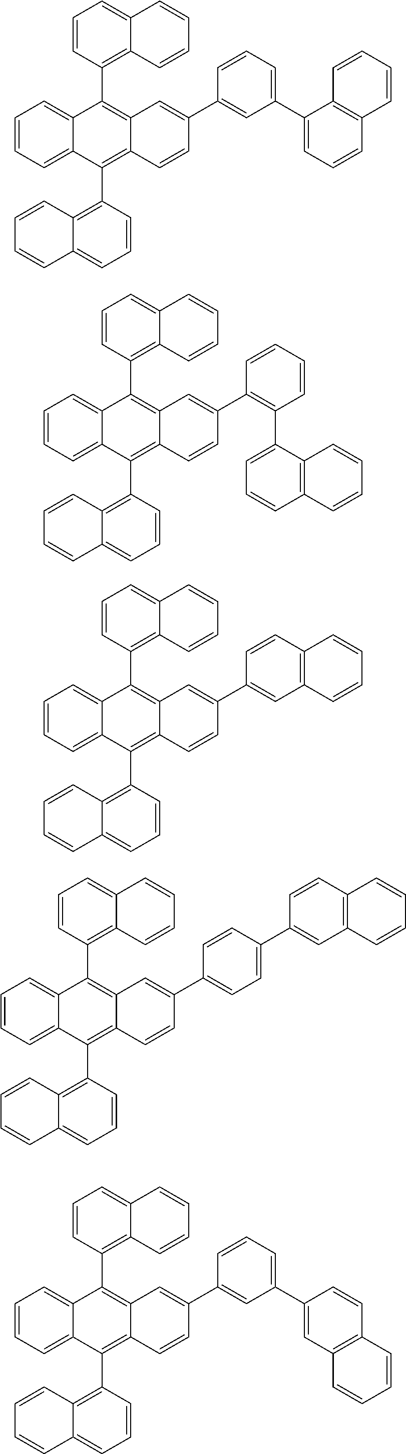

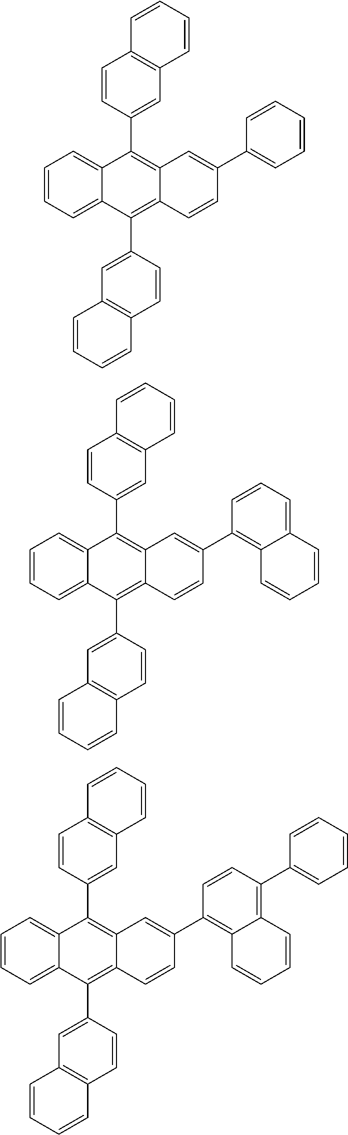

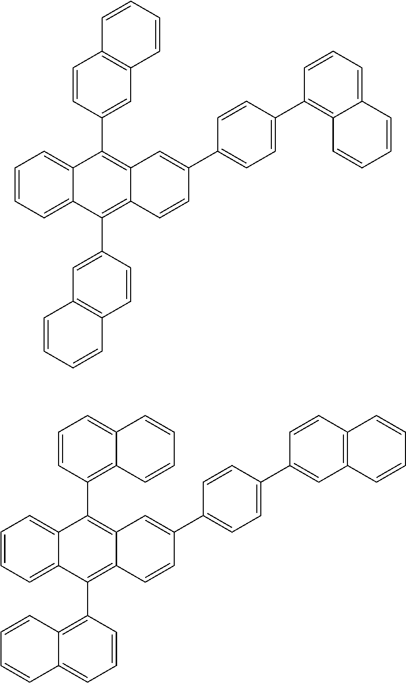

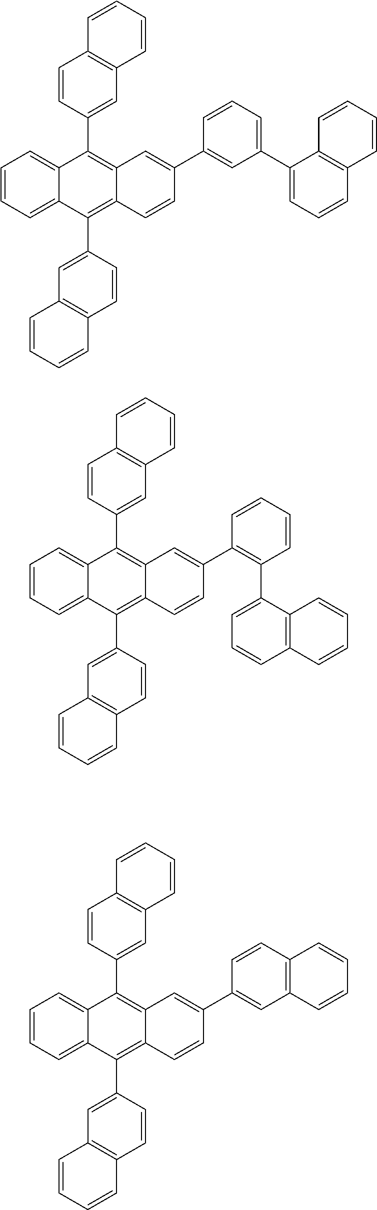

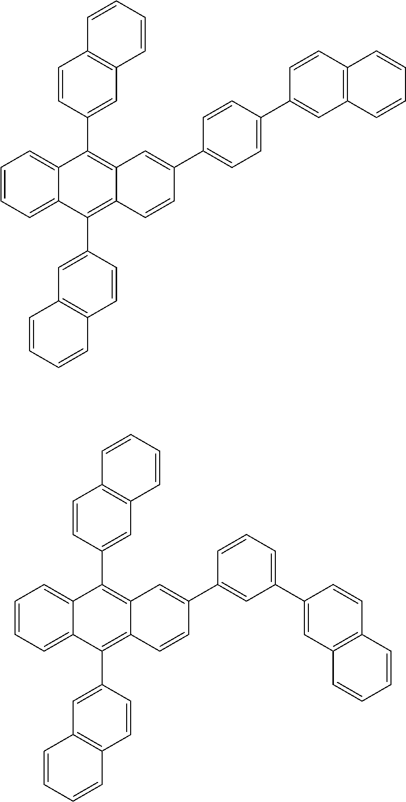

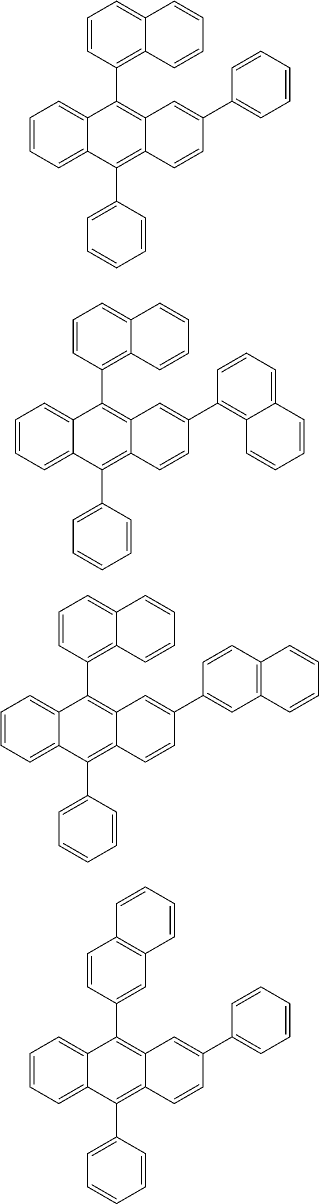

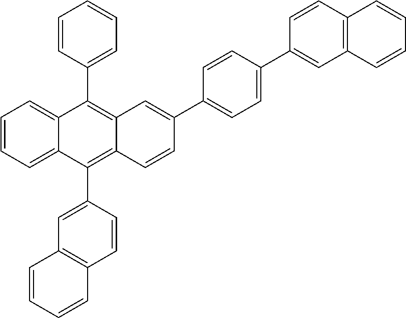 [SMILES: c1ccc(-c2c3ccccc3c(-c3ccc4ccccc4c3)c3ccc(-c4ccc(-c5ccc6ccccc6c5)cc4)cc23)cc1]

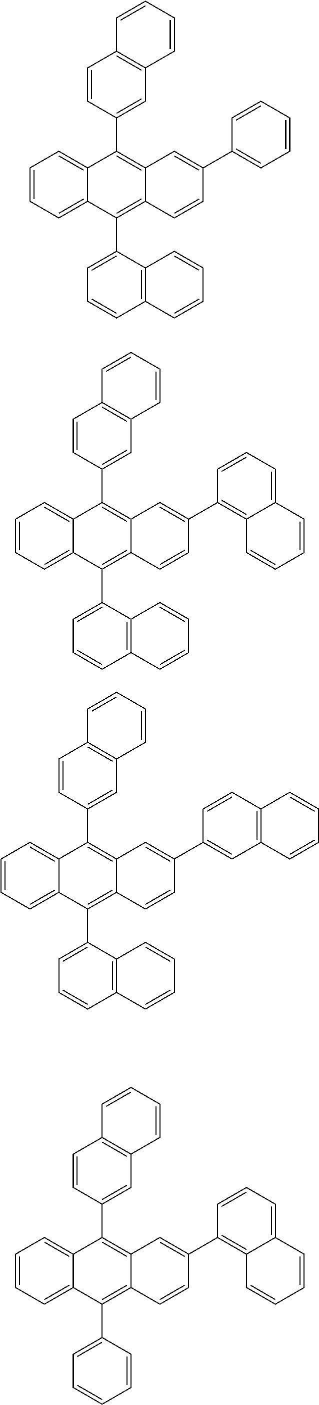

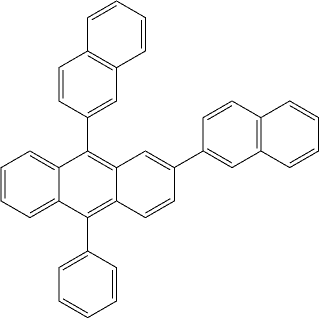 [SMILES: c1ccc(-c2c3ccccc3c(-c3ccc4ccccc4c3)c3cc(-c4ccc5ccccc5c4)ccc23)cc1]

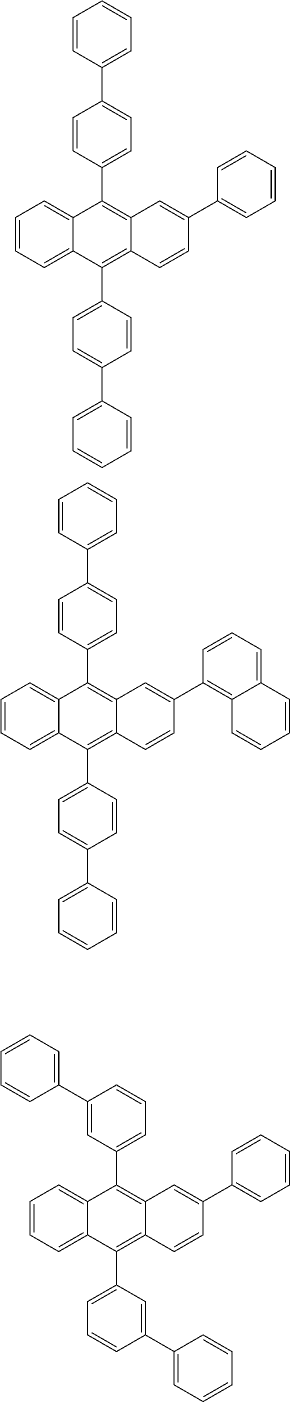

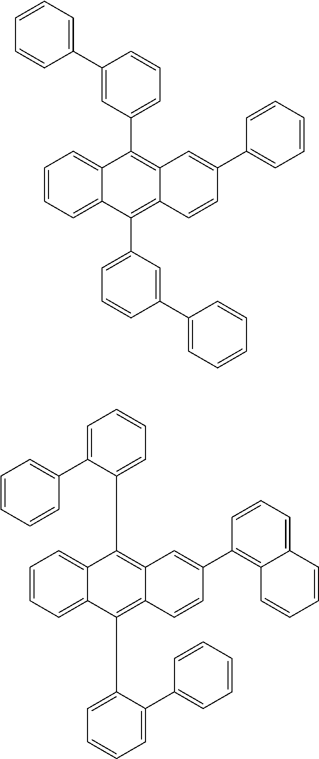

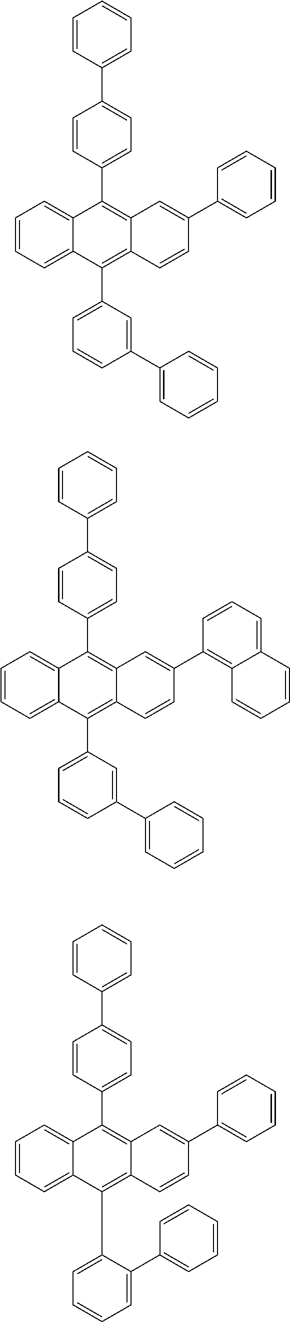

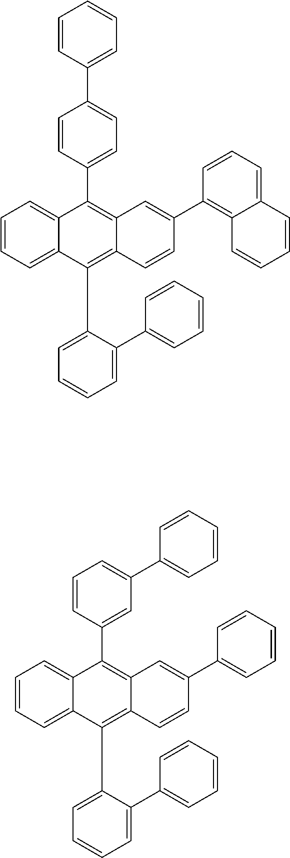

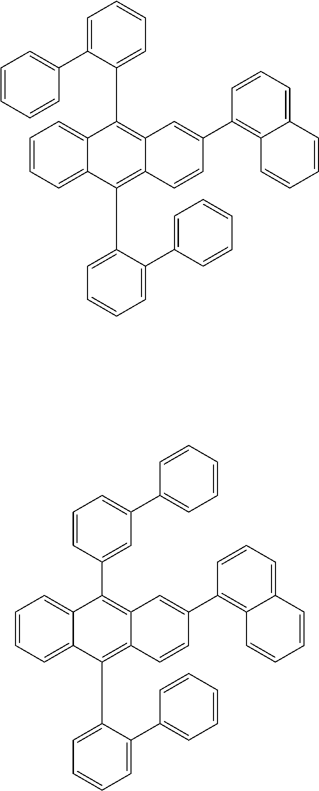

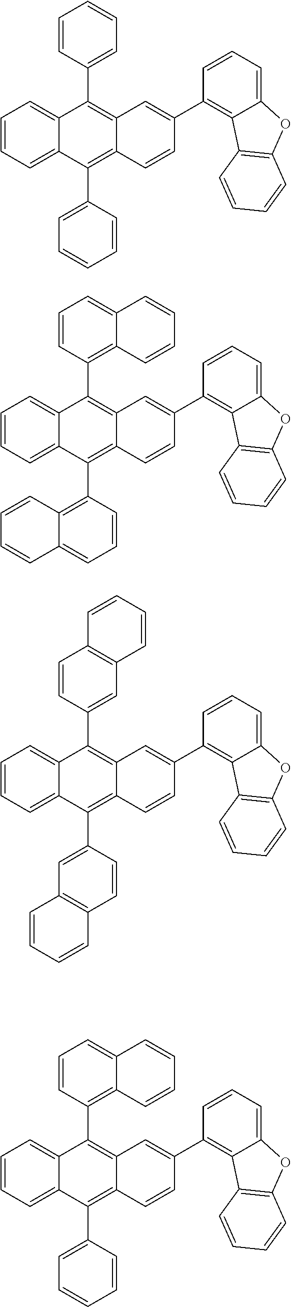

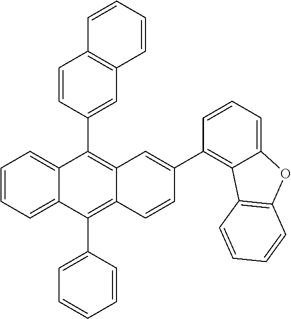 [SMILES: c1ccc(-c2c3ccccc3c(-c3ccc4ccccc4c3)c3cc(-c4cccc5oc6ccccc6c45)ccc23)cc1]

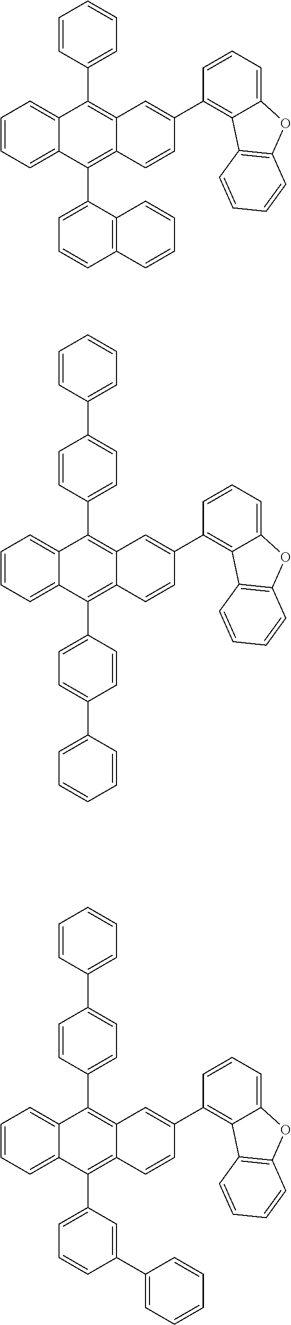 [SMILES: c1ccc(-c2c3ccccc3c(-c3cccc4ccccc34)c3ccc(-c4cccc5oc6ccccc6c45)cc23)cc1 ; c1ccc(-c2ccc(-c3c4ccccc4c(-c4ccc(-c5ccccc5)cc4)c4cc(-c5cccc6oc7ccccc7c56)ccc34)cc2)cc1 ; c1ccc(-c2ccc(-c3c4ccccc4c(-c4cccc(-c5ccccc5)c4)c4ccc(-c5cccc6oc7ccccc7c56)cc34)cc2)cc1]

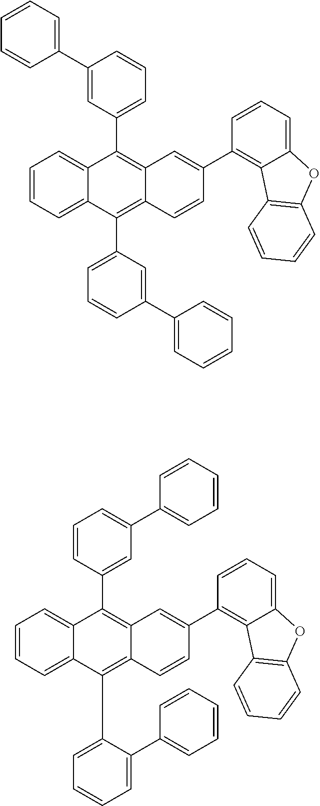 [SMILES: c1ccc(-c2cccc(-c3c4ccccc4c(-c4cccc(-c5ccccc5)c4)c4cc(-c5cccc6oc7ccccc7c56)ccc34)c2)cc1 ; c1ccc(-c2cccc(-c3c4ccccc4c(-c4ccccc4-c4ccccc4)c4ccc(-c5cccc6oc7ccccc7c56)cc34)c2)cc1]

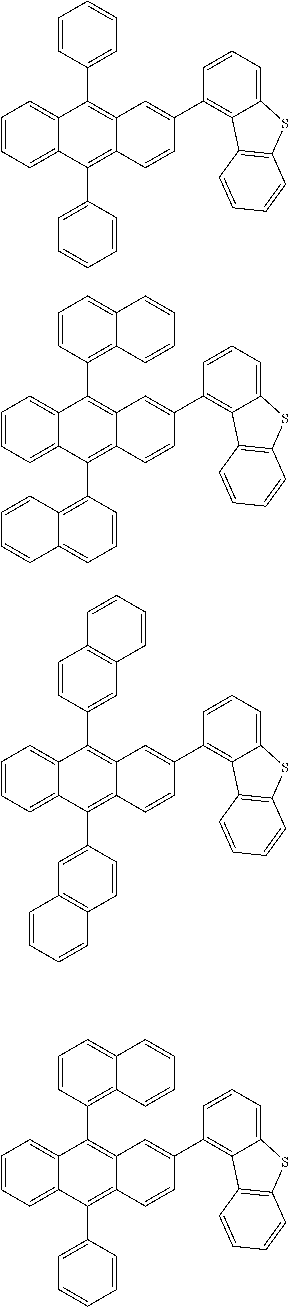

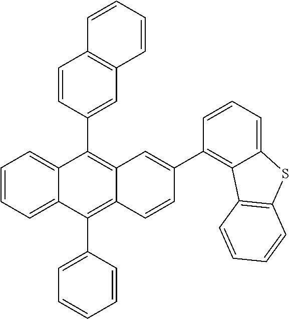 [SMILES: c1ccc(-c2c3ccccc3c(-c3ccc4ccccc4c3)c3cc(-c4cccc5sc6ccccc6c45)ccc23)cc1]

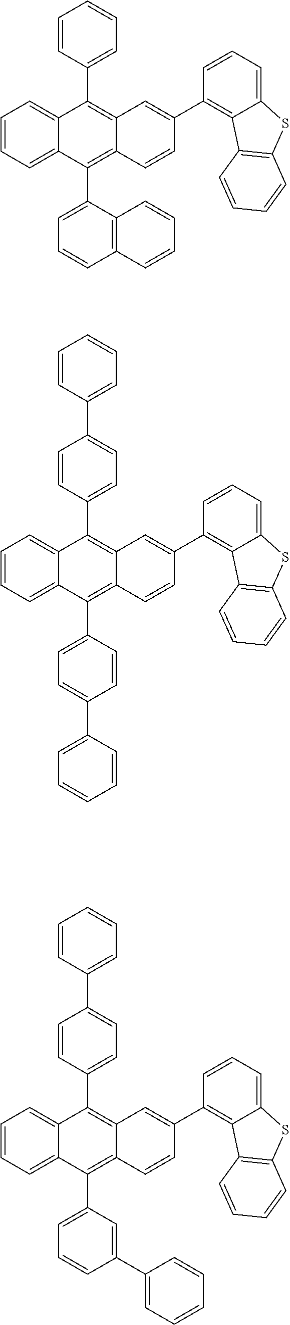 [SMILES: c1ccc(-c2c3ccccc3c(-c3cccc4ccccc34)c3ccc(-c4cccc5sc6ccccc6c45)cc23)cc1 ; c1ccc(-c2ccc(-c3c4ccccc4c(-c4ccc(-c5ccccc5)cc4)c4cc(-c5cccc6sc7ccccc7c56)ccc34)cc2)cc1 ; c1ccc(-c2ccc(-c3c4ccccc4c(-c4cccc(-c5ccccc5)c4)c4ccc(-c5cccc6sc7ccccc7c56)cc34)cc2)cc1]

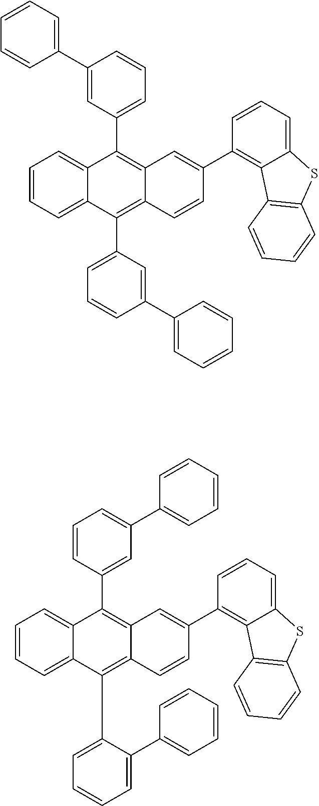 [SMILES: c1ccc(-c2cccc(-c3c4ccccc4c(-c4cccc(-c5ccccc5)c4)c4cc(-c5cccc6sc7ccccc7c56)ccc34)c2)cc1 ; c1ccc(-c2cccc(-c3c4ccccc4c(-c4ccccc4-c4ccccc4)c4ccc(-c5cccc6sc7ccccc7c56)cc34)c2)cc1]

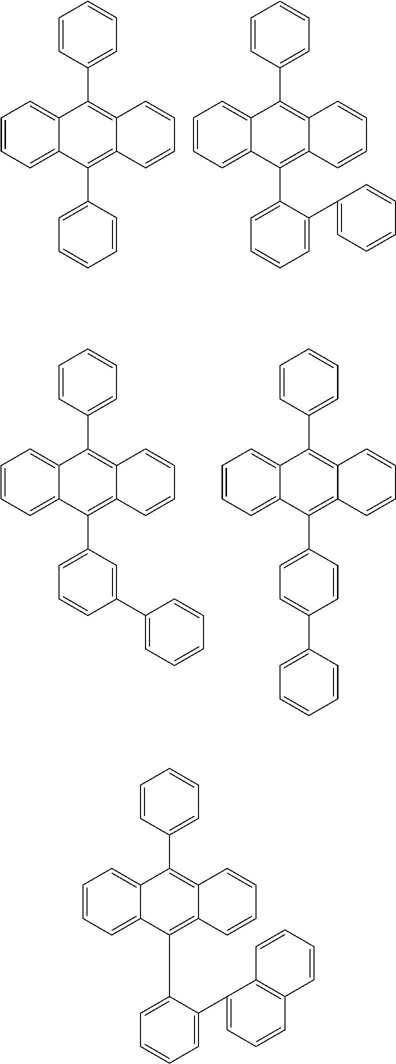

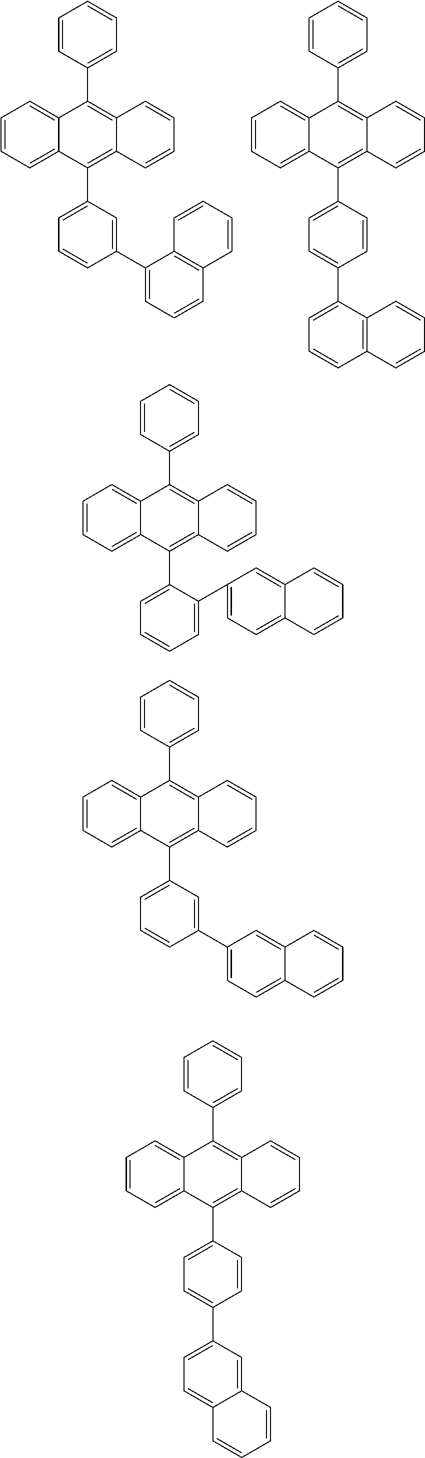

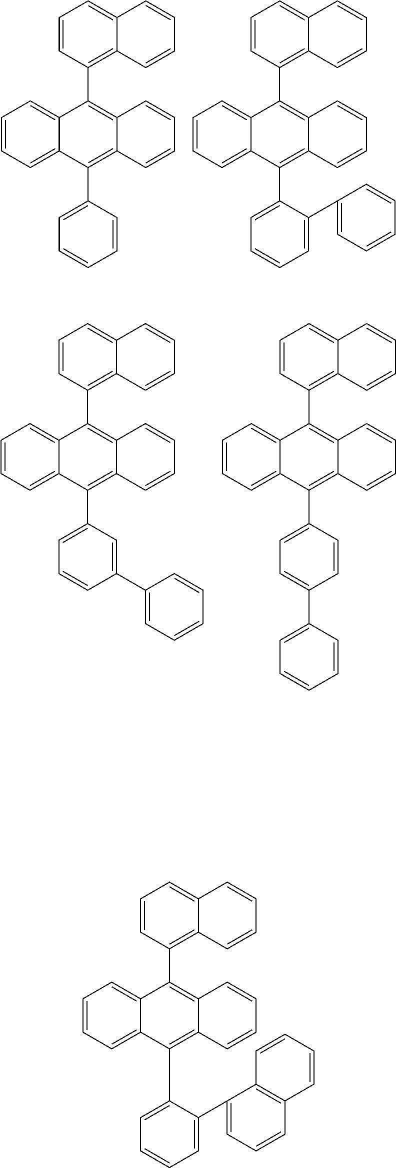

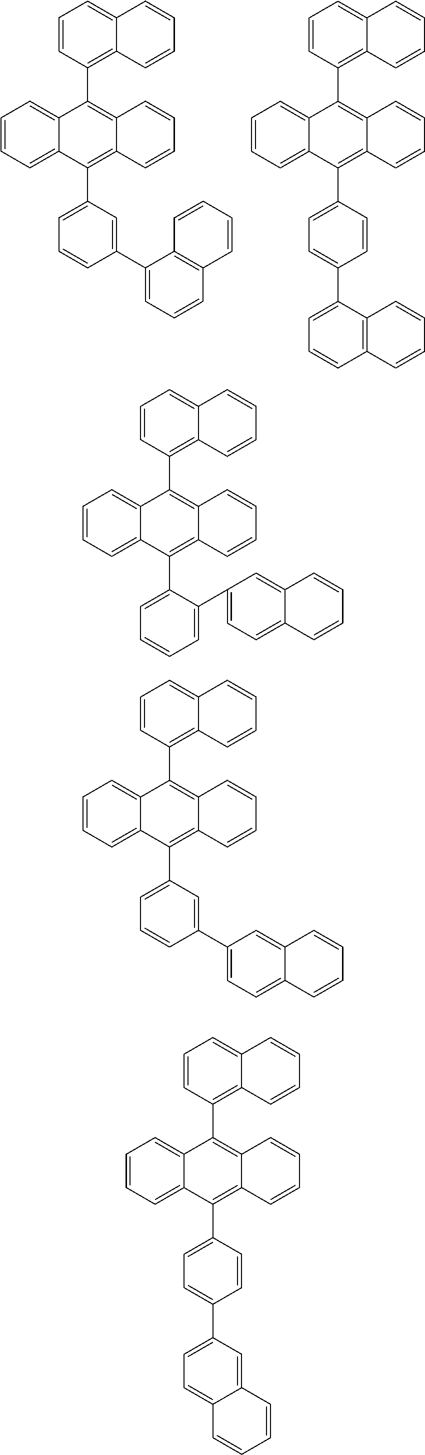

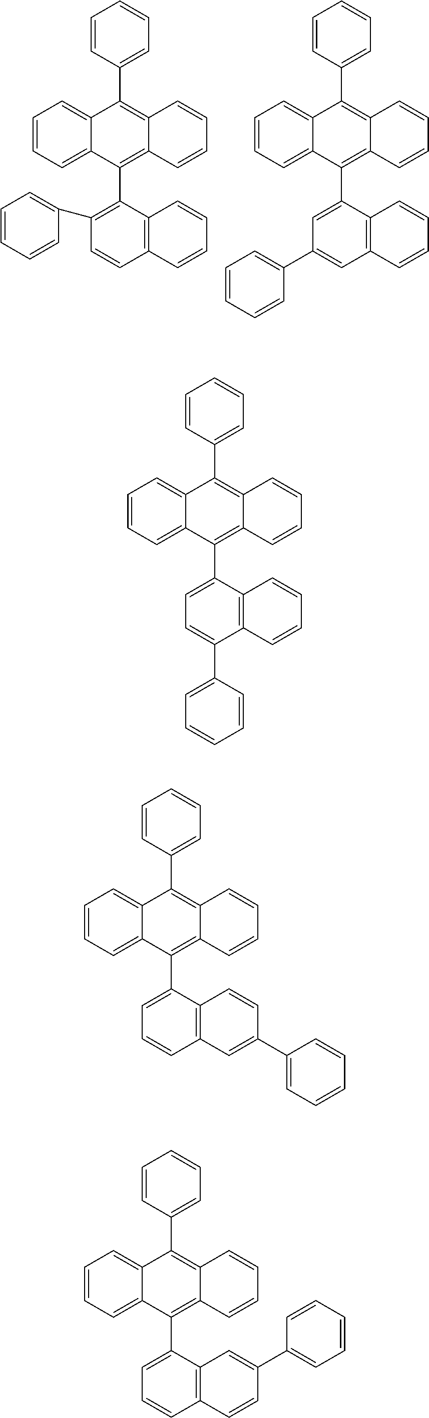

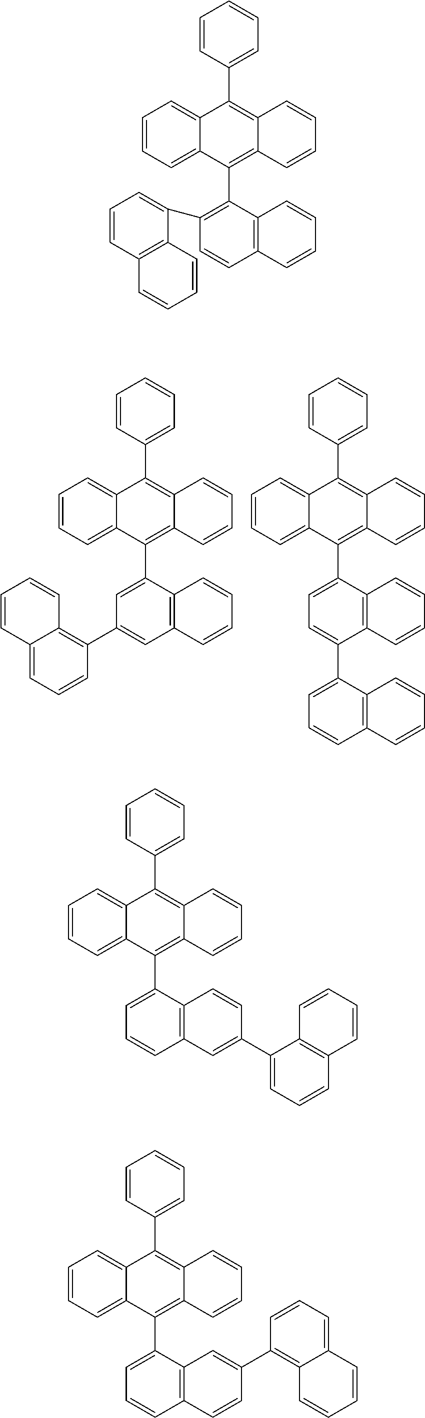

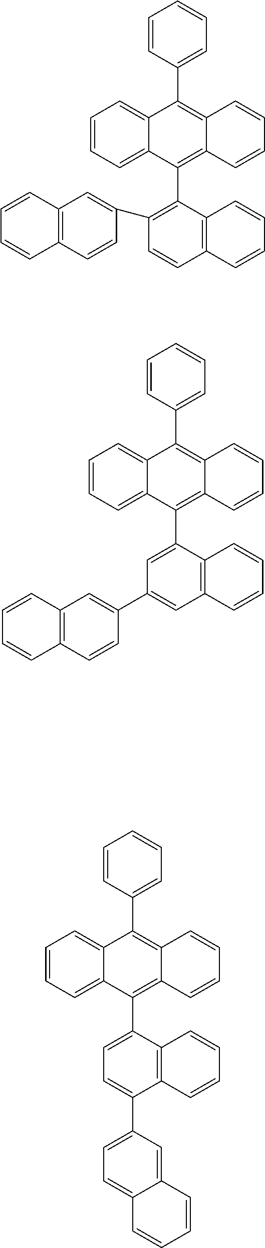

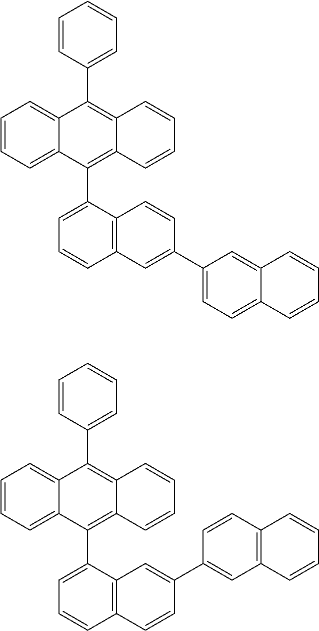

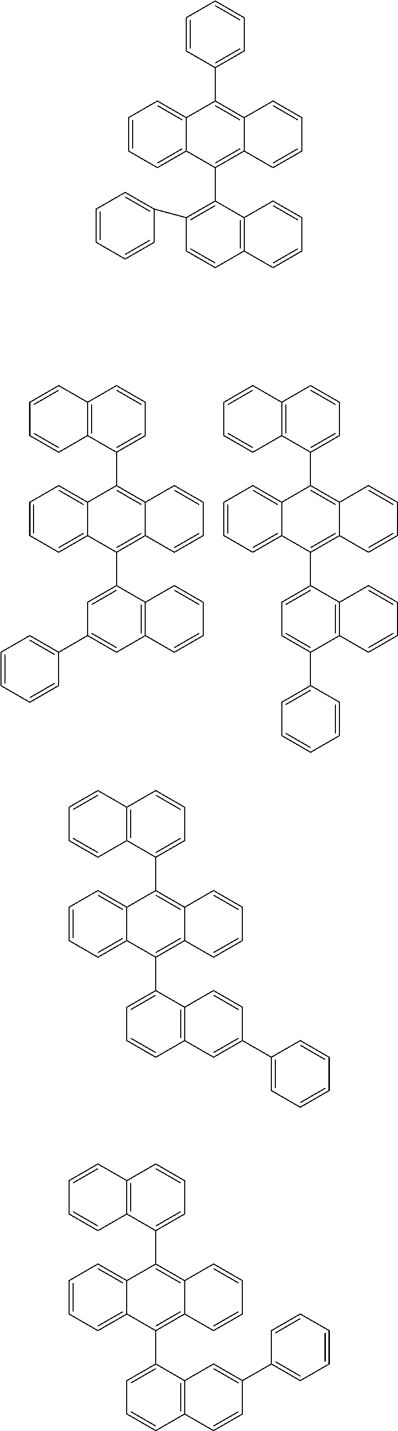

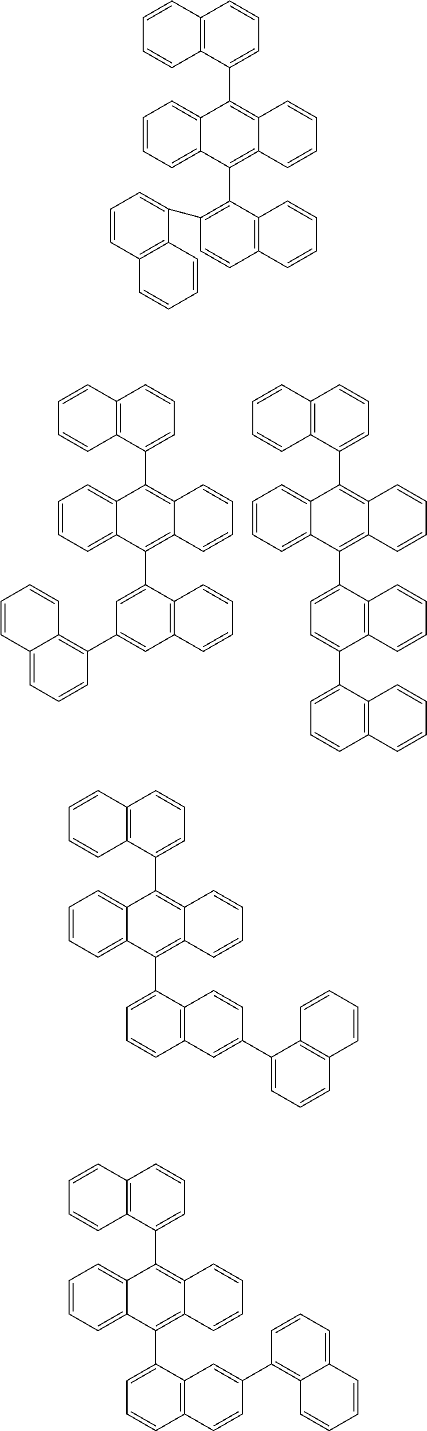

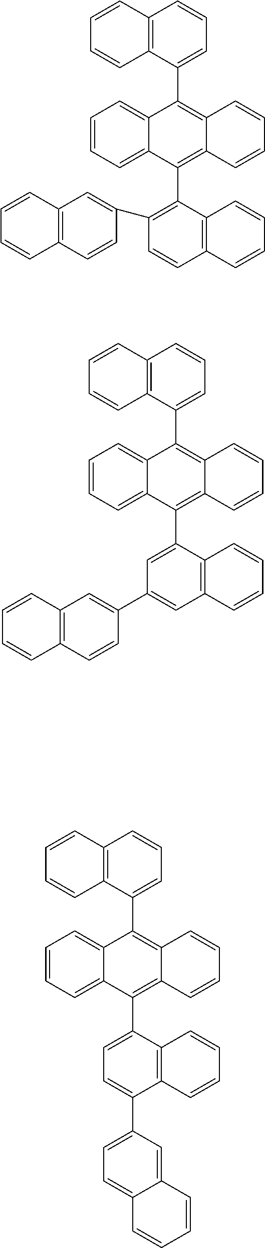

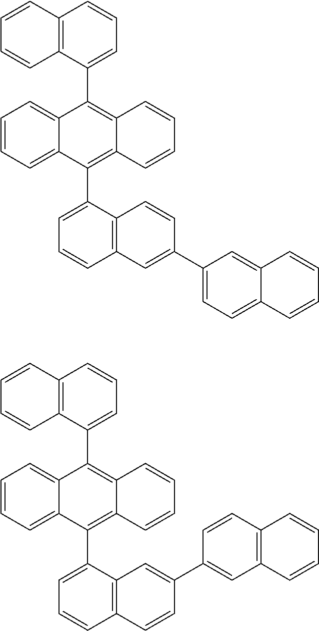

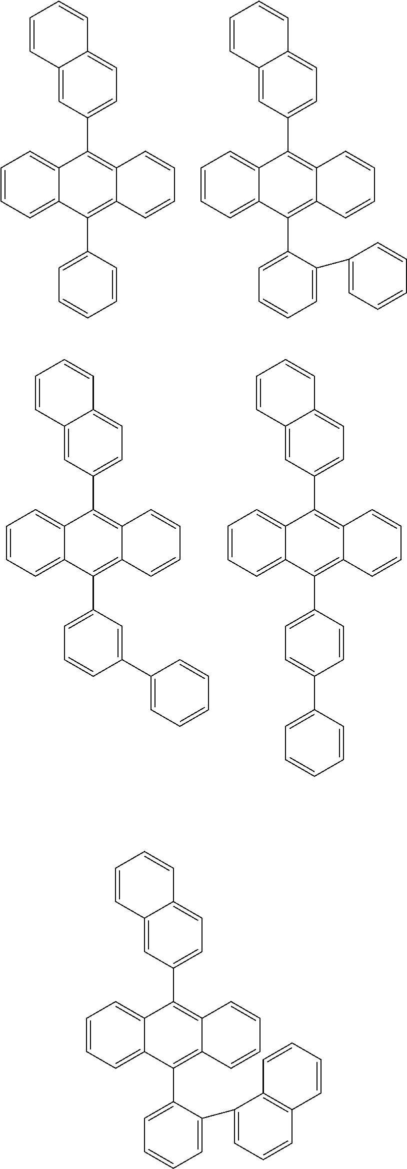

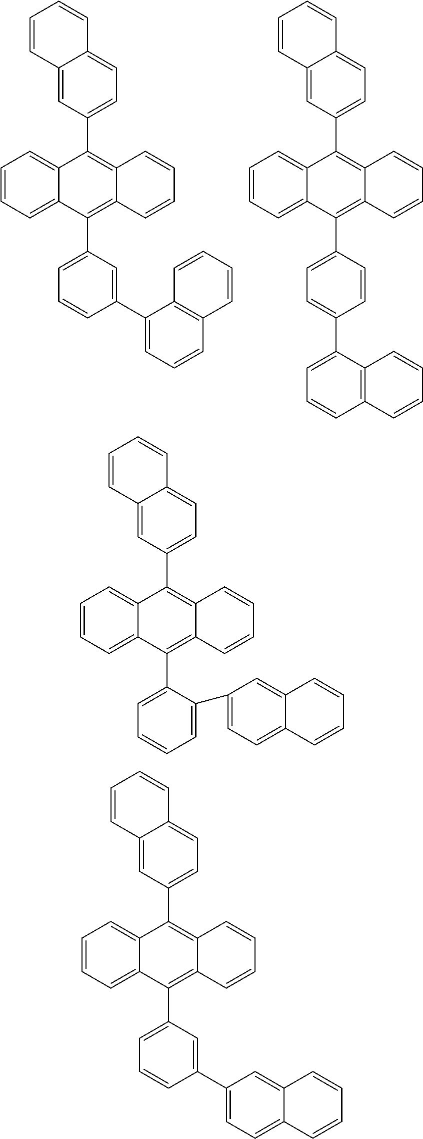

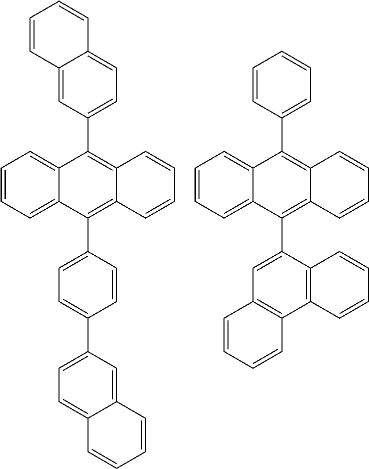

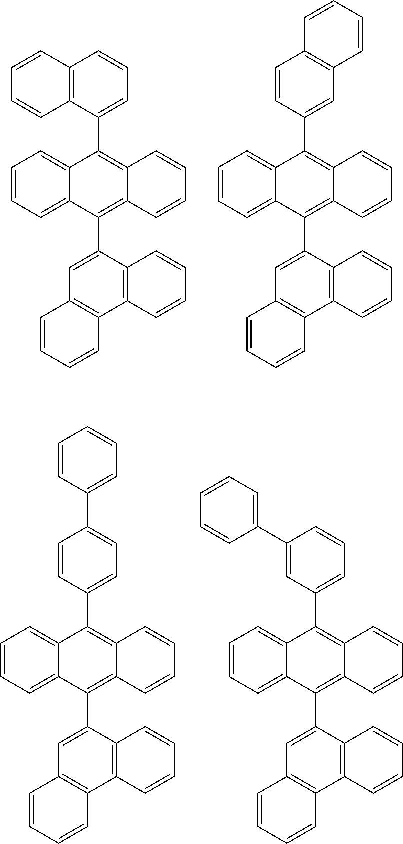

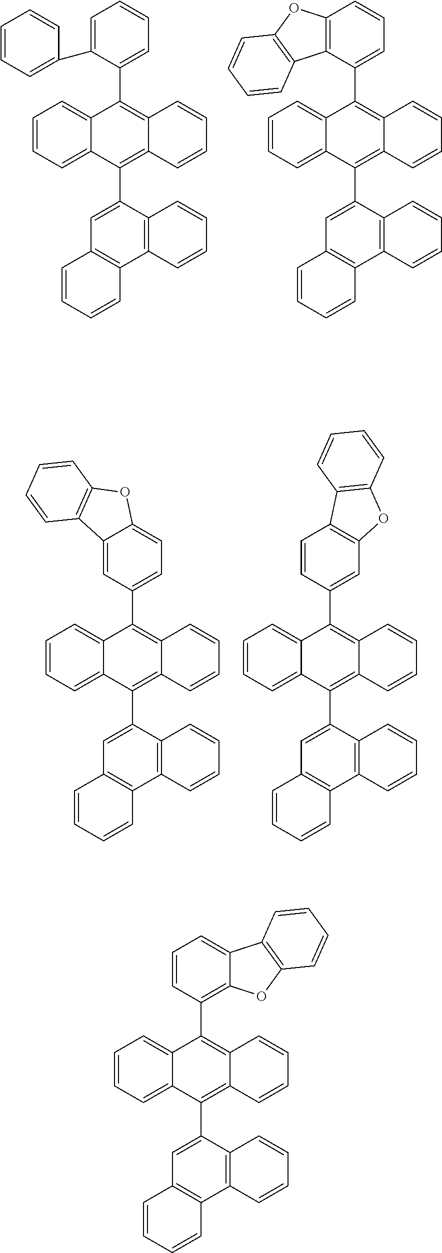

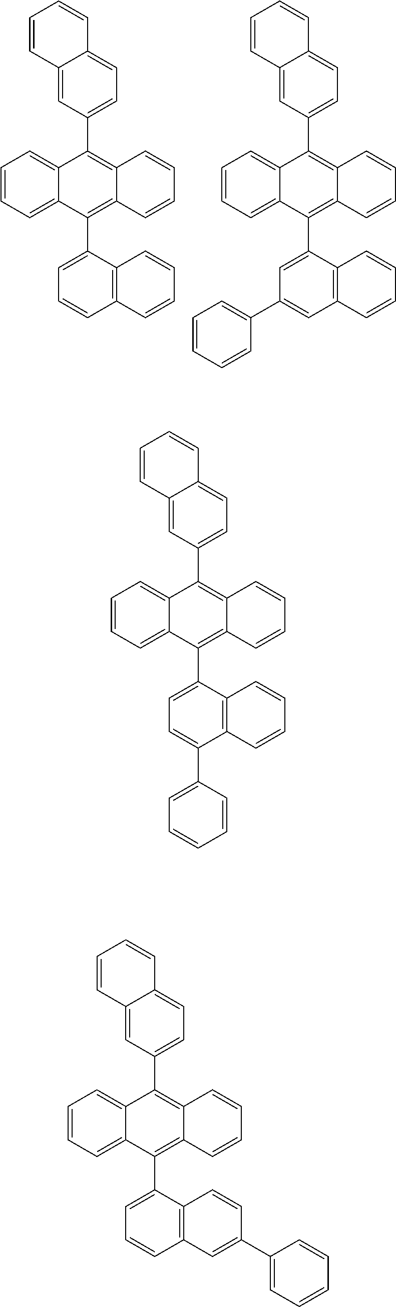

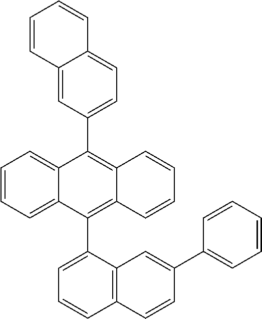 [SMILES: c1ccc(-c2ccc3cccc(-c4c5ccccc5c(-c5ccc6ccccc6c5)c5ccccc45)c3c2)cc1]

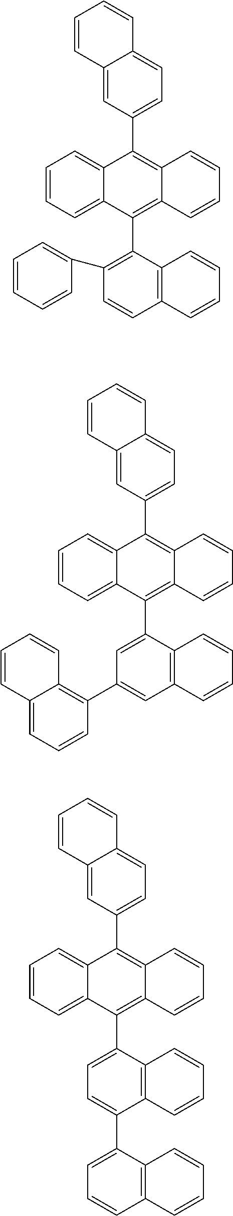

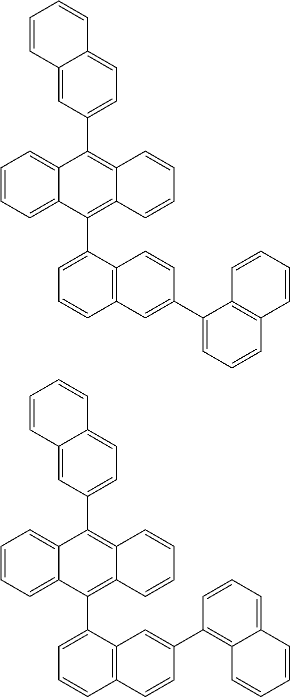

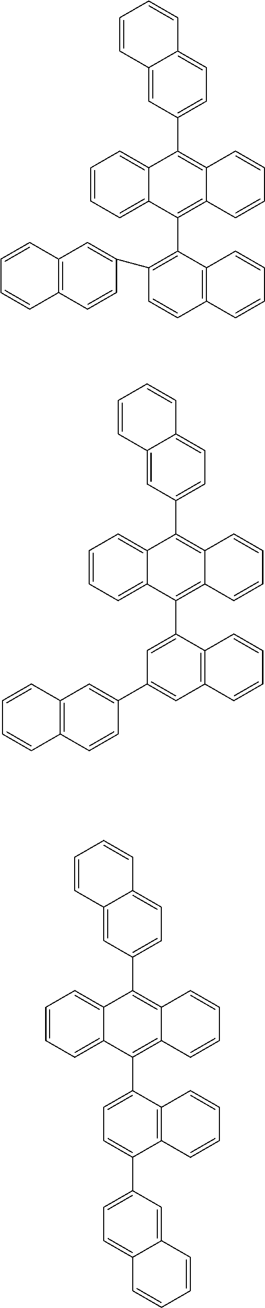

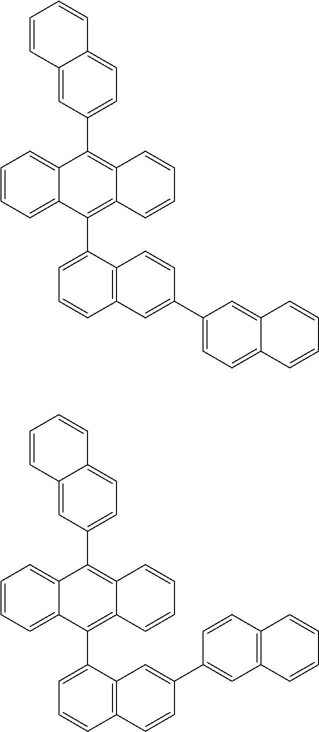

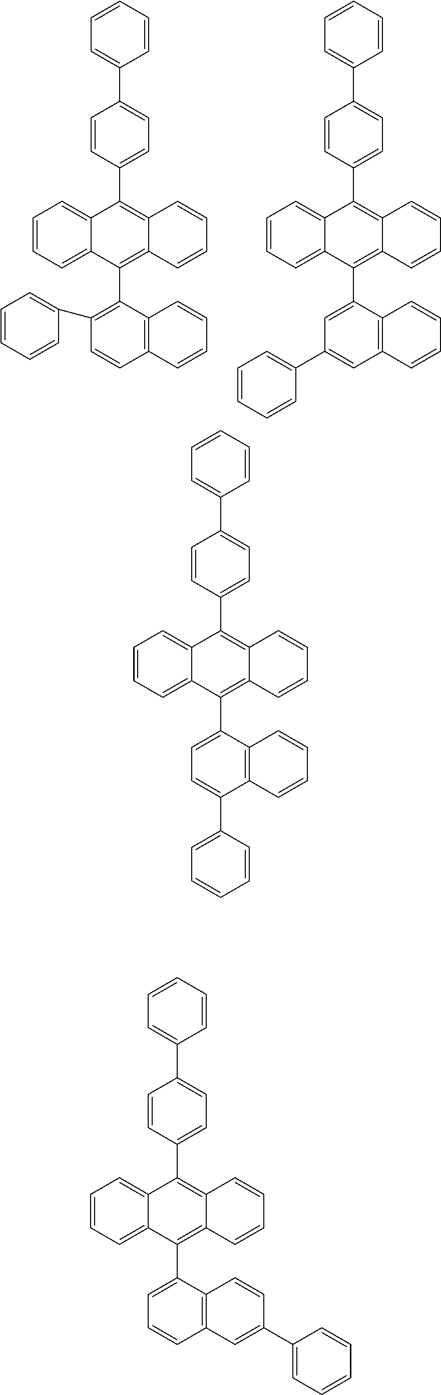

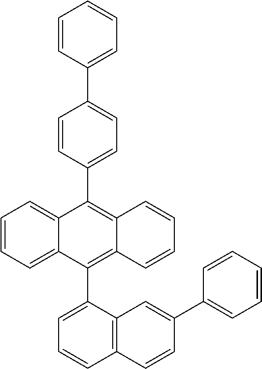 [SMILES: c1ccc(-c2ccc(-c3c4ccccc4c(-c4cccc5ccc(-c6ccccc6)cc45)c4ccccc34)cc2)cc1]

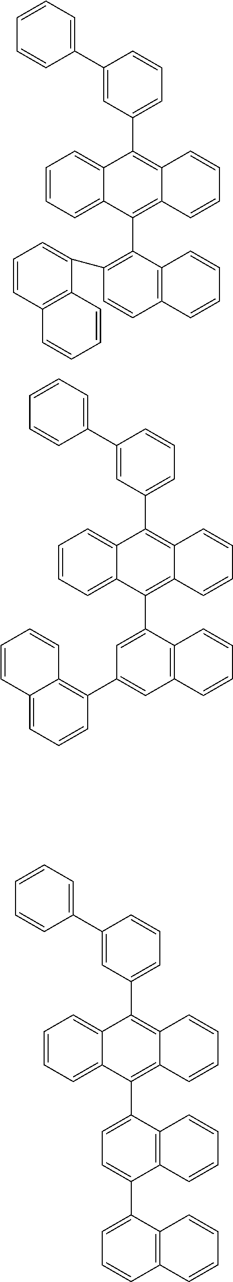

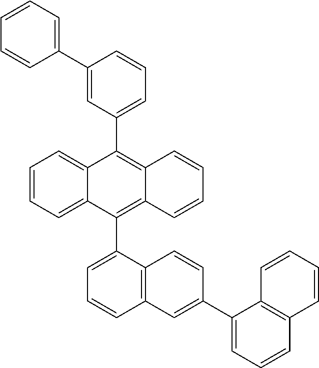 [SMILES: c1ccc(-c2cccc(-c3c4ccccc4c(-c4cccc5cc(-c6cccc7ccccc67)ccc45)c4ccccc34)c2)cc1]

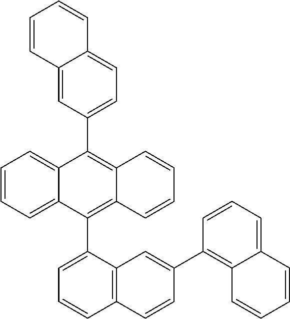 [SMILES: c1ccc2cc(-c3c4ccccc4c(-c4cccc5ccc(-c6cccc7ccccc67)cc45)c4ccccc34)ccc2c1]

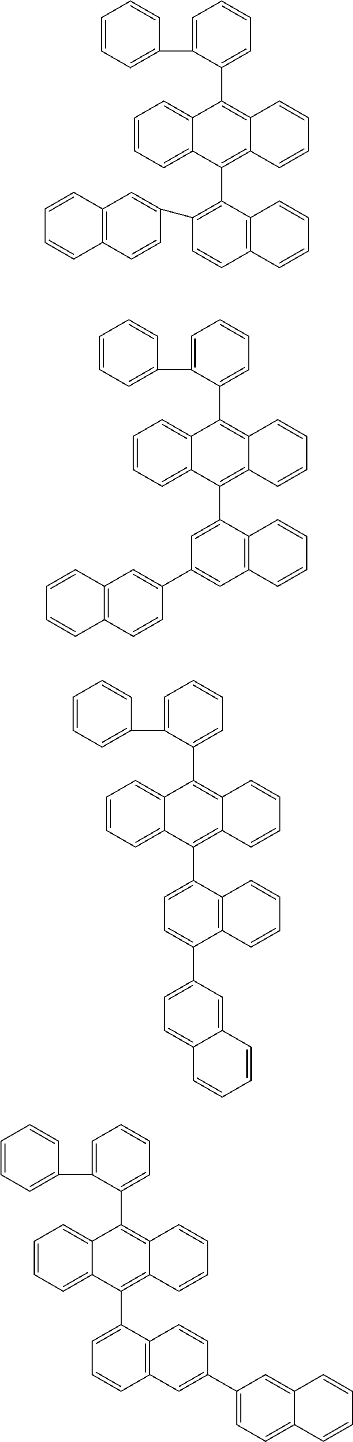

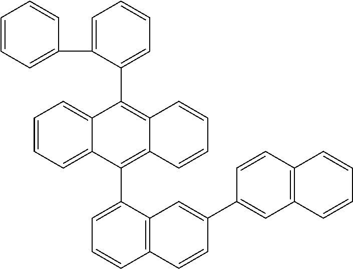 [SMILES: c1ccc(-c2ccccc2-c2c3ccccc3c(-c3cccc4ccc(-c5ccc6ccccc6c5)cc34)c3ccccc23)cc1]

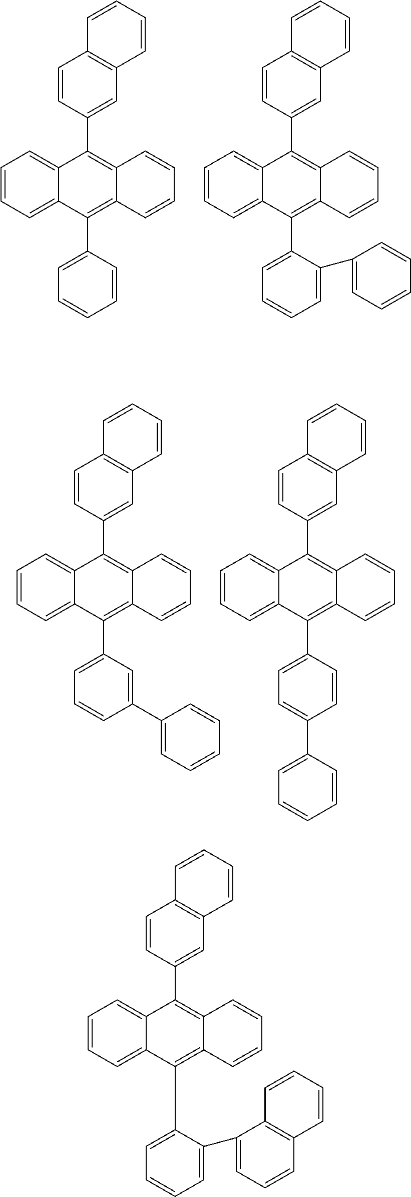

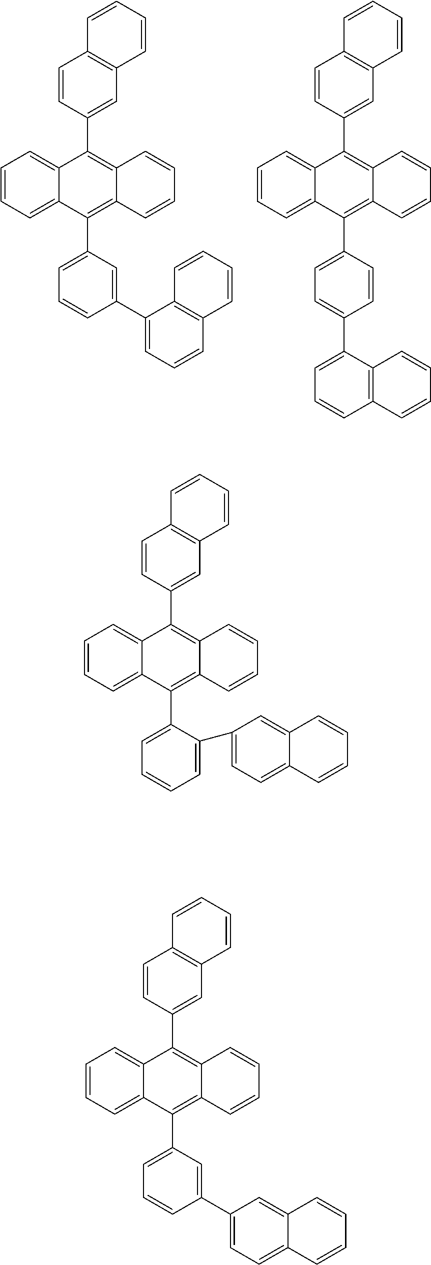

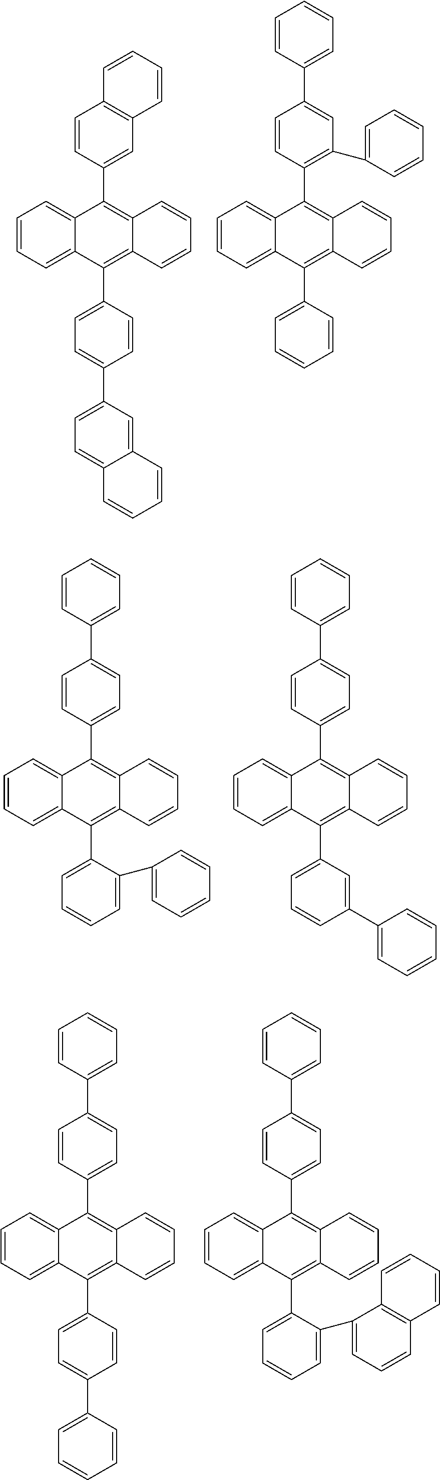

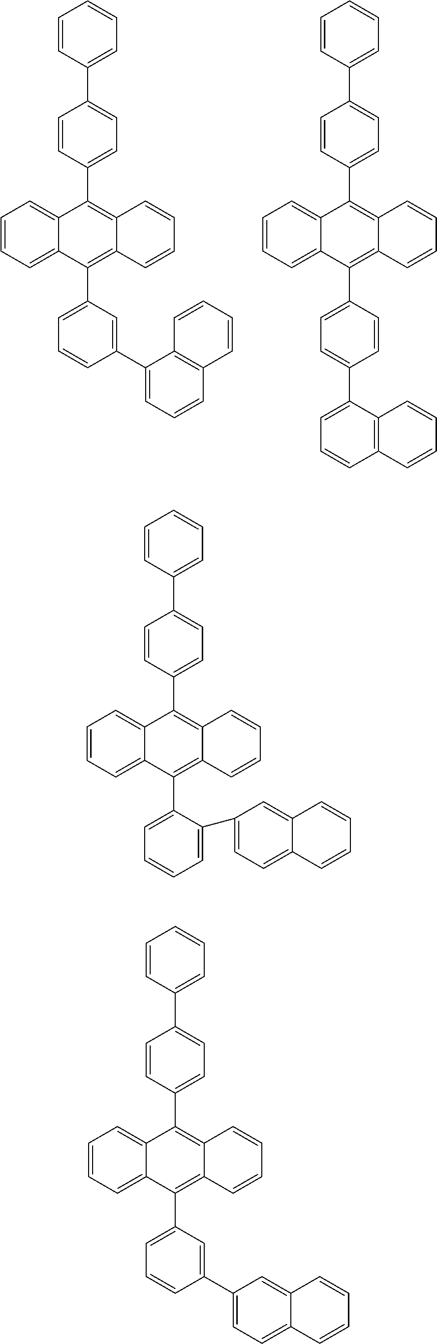

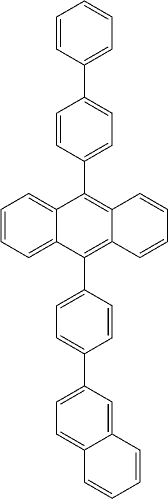 [SMILES: c1ccc(-c2ccc(-c3c4ccccc4c(-c4ccc(-c5ccc6ccccc6c5)cc4)c4ccccc34)cc2)cc1]

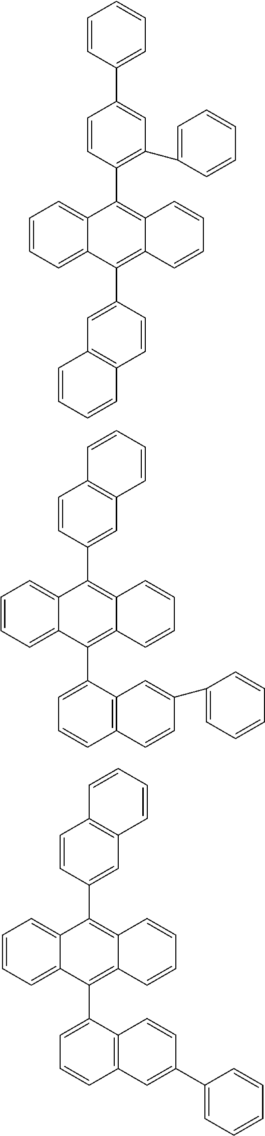

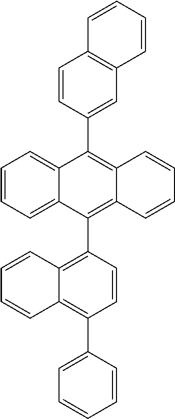 [SMILES: c1ccc(-c2ccc(-c3c4ccccc4c(-c4ccc5ccccc5c4)c4ccccc34)c3ccccc23)cc1]

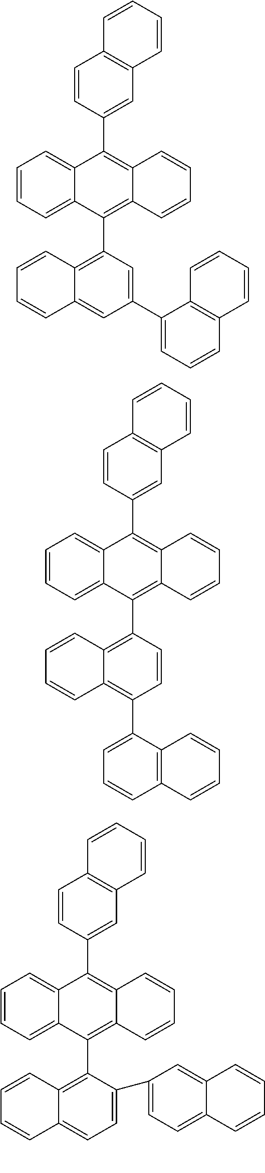

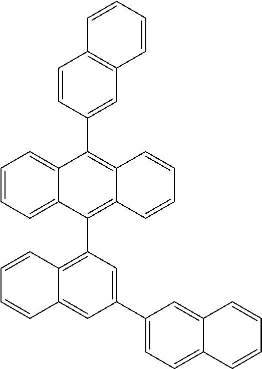 [SMILES: c1ccc2cc(-c3cc(-c4c5ccccc5c(-c5ccc6ccccc6c5)c5ccccc45)c4ccccc4c3)ccc2c1]

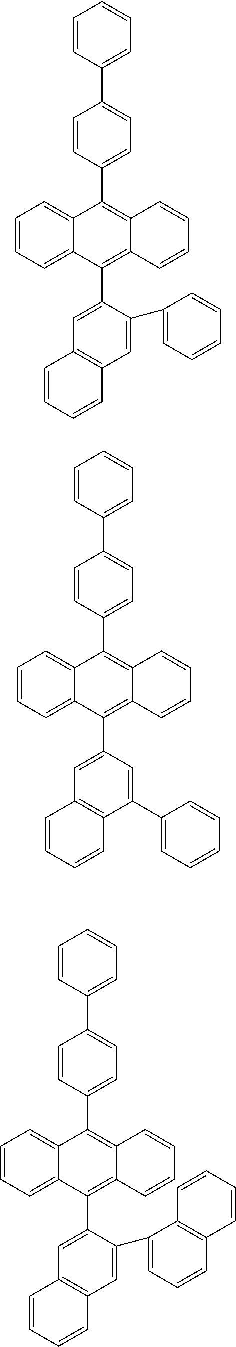

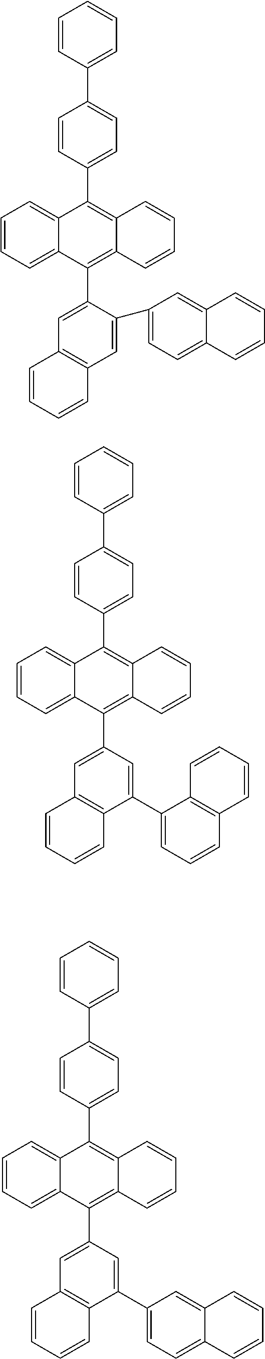

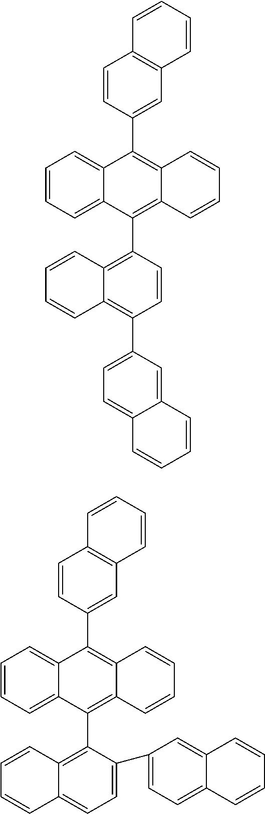

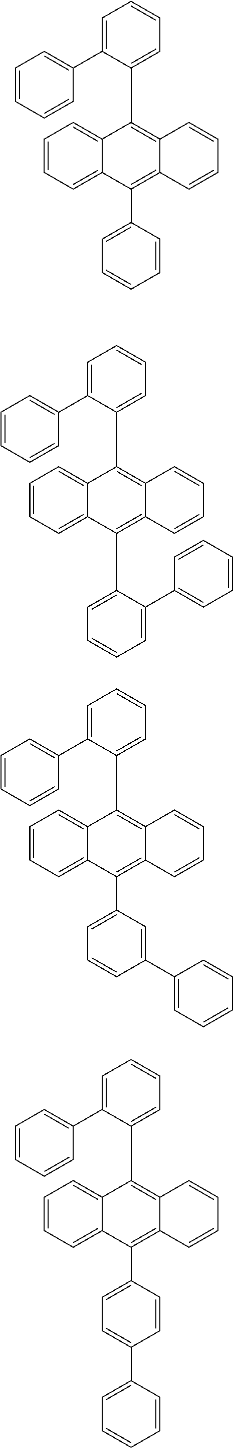

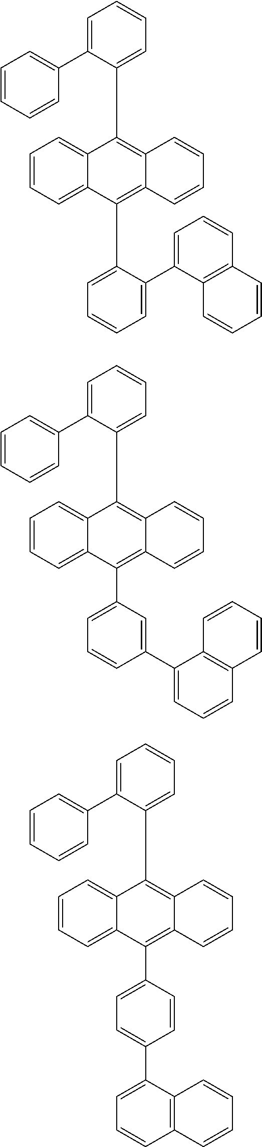

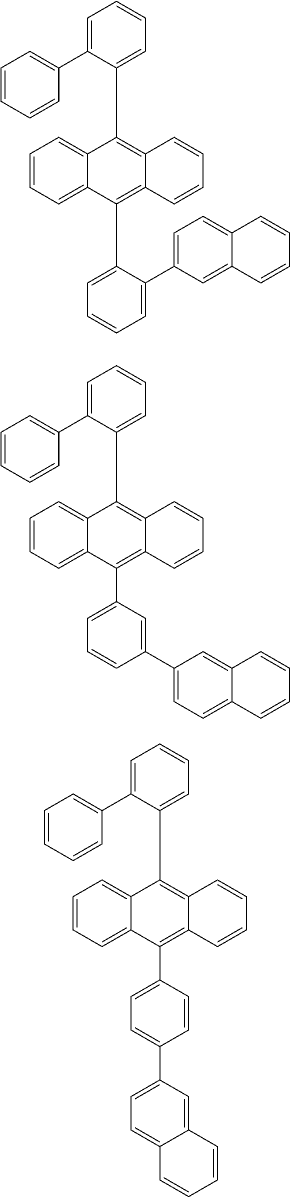

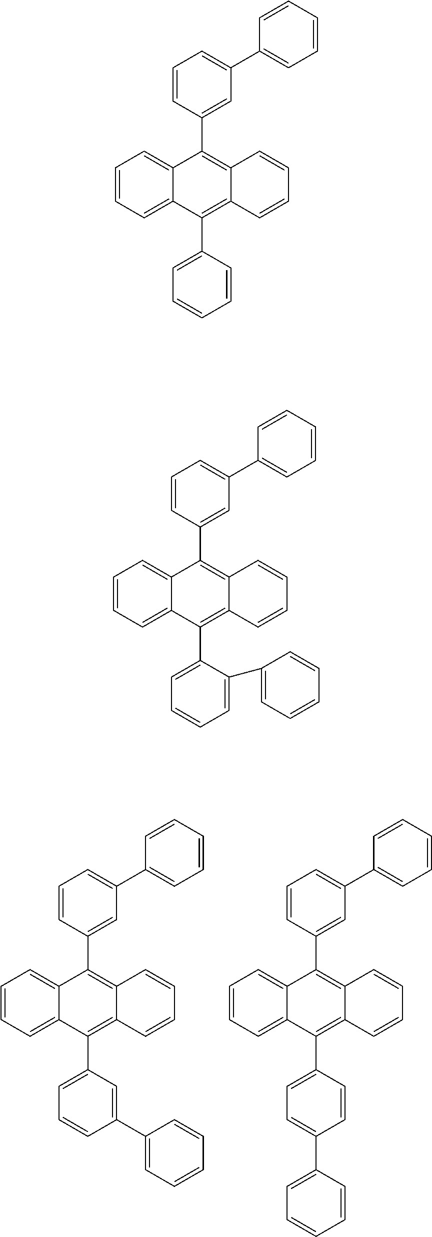

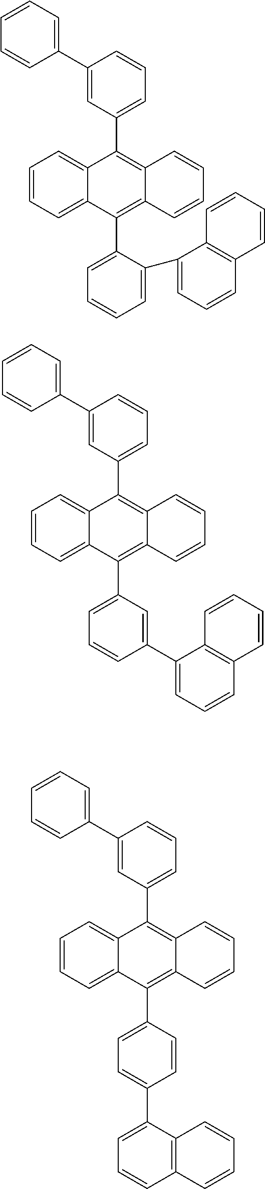

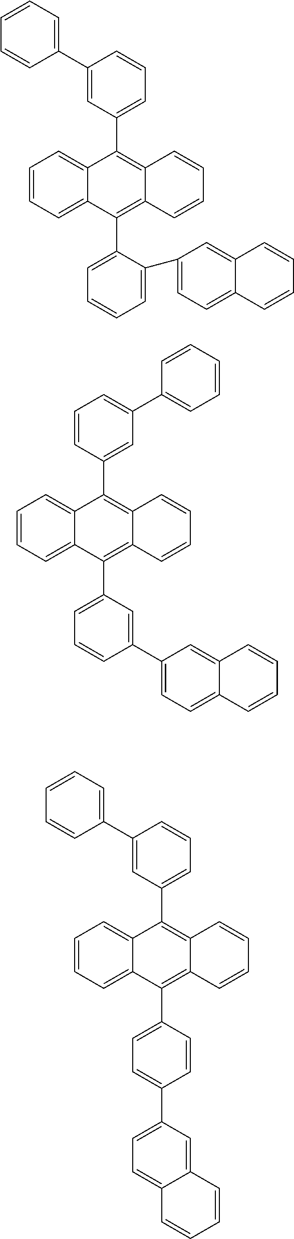

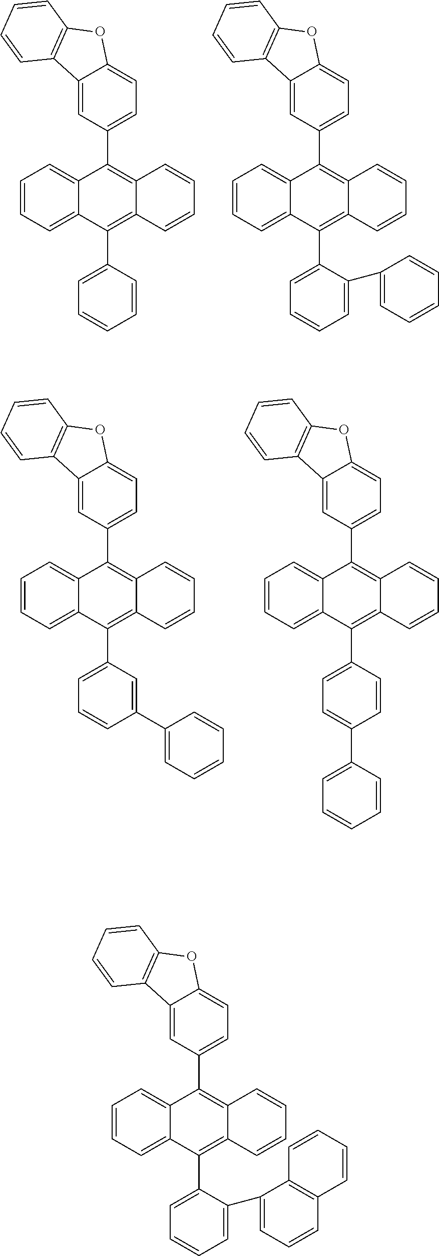

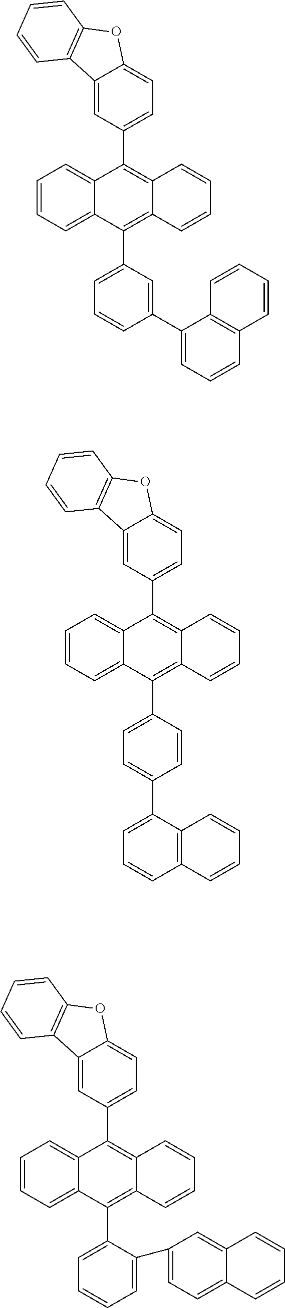 [SMILES: c1cc(-c2cccc3ccccc23)cc(-c2c3ccccc3c(-c3ccc4oc5ccccc5c4c3)c3ccccc23)c1 ; c1ccc2c(-c3ccc(-c4c5ccccc5c(-c5ccc6oc7ccccc7c6c5)c5ccccc45)cc3)cccc2c1 ; c1ccc(-c2c3ccccc3c(-c3ccc4oc5ccccc5c4c3)c3ccccc23)c(-c2ccc3ccccc3c2)c1]

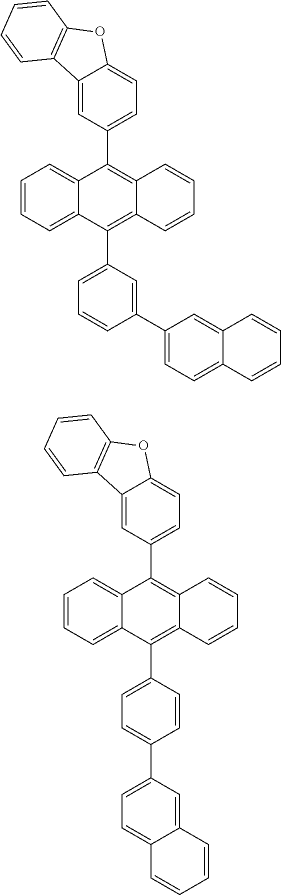 [SMILES: c1cc(-c2ccc3ccccc3c2)cc(-c2c3ccccc3c(-c3ccc4oc5ccccc5c4c3)c3ccccc23)c1 ; c1ccc2cc(-c3ccc(-c4c5ccccc5c(-c5ccc6oc7ccccc7c6c5)c5ccccc45)cc3)ccc2c1]

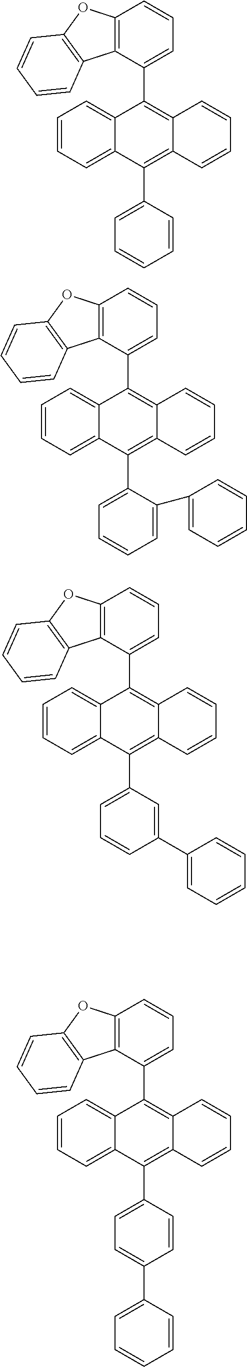

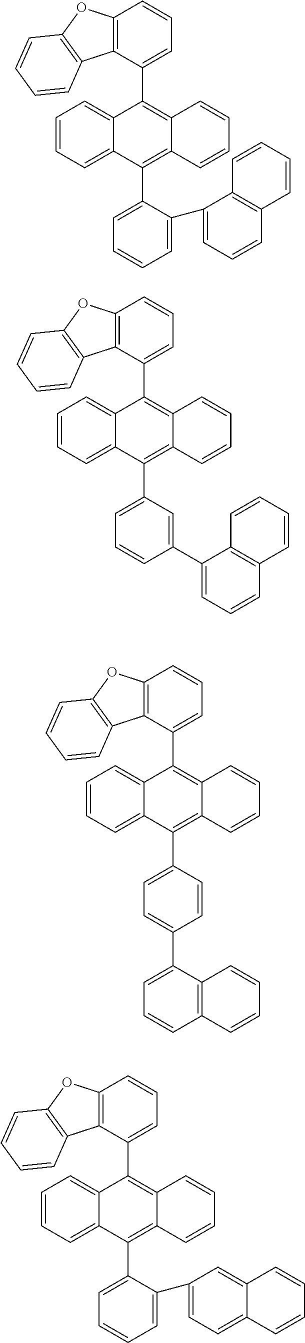

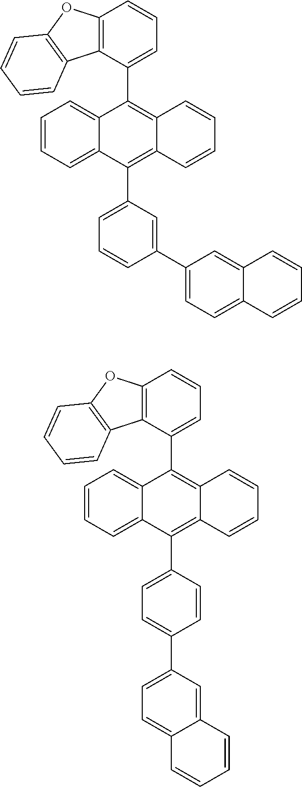 [SMILES: c1cc(-c2ccc3ccccc3c2)cc(-c2c3ccccc3c(-c3cccc4oc5ccccc5c34)c3ccccc23)c1 ; c1ccc2cc(-c3ccc(-c4c5ccccc5c(-c5cccc6oc7ccccc7c56)c5ccccc45)cc3)ccc2c1]

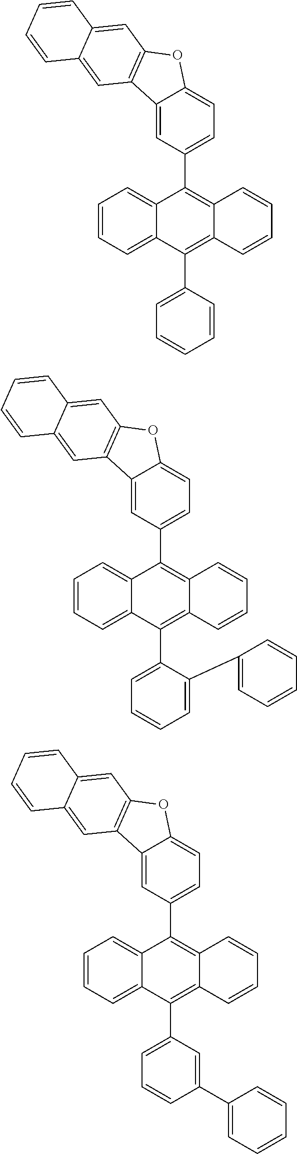 [SMILES: c1ccc(-c2c3ccccc3c(-c3ccc4oc5cc6ccccc6cc5c4c3)c3ccccc23)cc1 ; c1ccc(-c2ccccc2-c2c3ccccc3c(-c3ccc4oc5cc6ccccc6cc5c4c3)c3ccccc23)cc1 ; c1ccc(-c2cccc(-c3c4ccccc4c(-c4ccc5oc6cc7ccccc7cc6c5c4)c4ccccc34)c2)cc1]

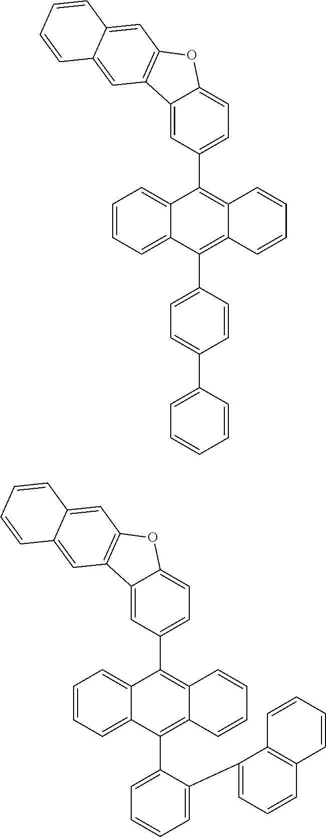 [SMILES: c1ccc(-c2ccc(-c3c4ccccc4c(-c4ccc5oc6cc7ccccc7cc6c5c4)c4ccccc34)cc2)cc1 ; c1ccc(-c2c3ccccc3c(-c3ccc4oc5cc6ccccc6cc5c4c3)c3ccccc23)c(-c2cccc3ccccc23)c1]

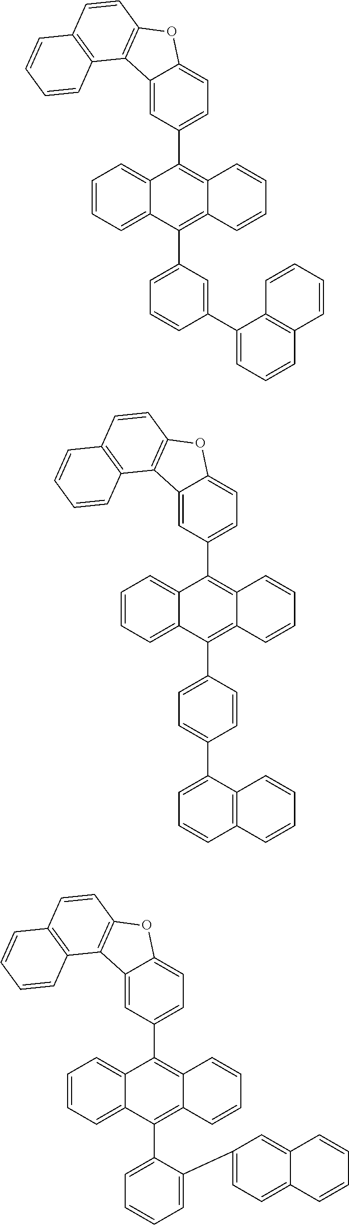 [SMILES: c1cc(-c2cccc3ccccc23)cc(-c2c3ccccc3c(-c3ccc4oc5ccc6ccccc6c5c4c3)c3ccccc23)c1 ; c1ccc2c(-c3ccc(-c4c5ccccc5c(-c5ccc6oc7ccc8ccccc8c7c6c5)c5ccccc45)cc3)cccc2c1 ; c1ccc(-c2c3ccccc3c(-c3ccc4oc5ccc6ccccc6c5c4c3)c3ccccc23)c(-c2ccc3ccccc3c2)c1]

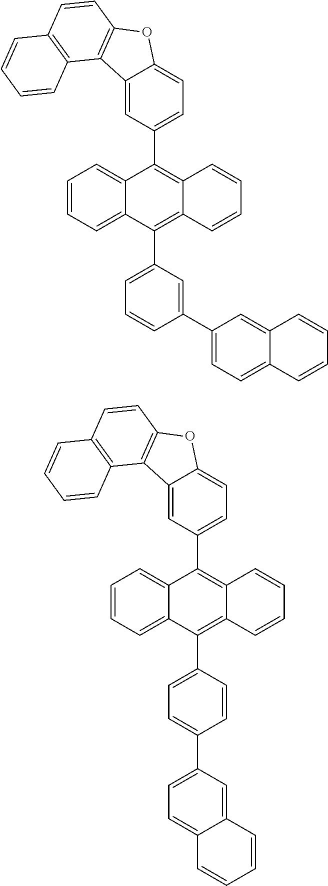 [SMILES: c1cc(-c2ccc3ccccc3c2)cc(-c2c3ccccc3c(-c3ccc4oc5ccc6ccccc6c5c4c3)c3ccccc23)c1 ; c1ccc2cc(-c3ccc(-c4c5ccccc5c(-c5ccc6oc7ccc8ccccc8c7c6c5)c5ccccc45)cc3)ccc2c1]

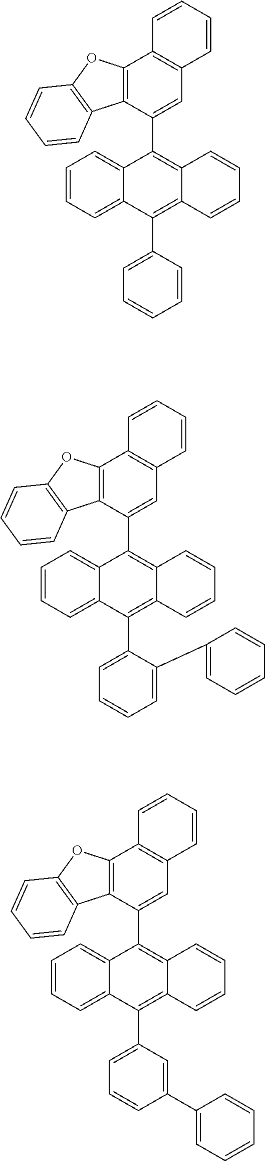 [SMILES: c1ccc(-c2c3ccccc3c(-c3cc4ccccc4c4oc5ccccc5c34)c3ccccc23)cc1 ; c1ccc(-c2ccccc2-c2c3ccccc3c(-c3cc4ccccc4c4oc5ccccc5c34)c3ccccc23)cc1 ; c1ccc(-c2cccc(-c3c4ccccc4c(-c4cc5ccccc5c5oc6ccccc6c45)c4ccccc34)c2)cc1]

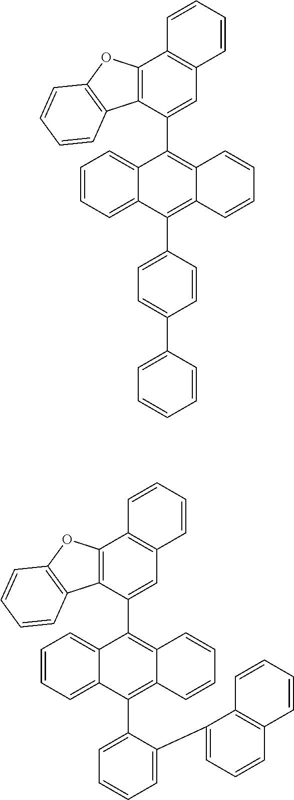 [SMILES: c1ccc(-c2ccc(-c3c4ccccc4c(-c4cc5ccccc5c5oc6ccccc6c45)c4ccccc34)cc2)cc1 ; c1ccc(-c2c3ccccc3c(-c3cc4ccccc4c4oc5ccccc5c34)c3ccccc23)c(-c2cccc3ccccc23)c1]

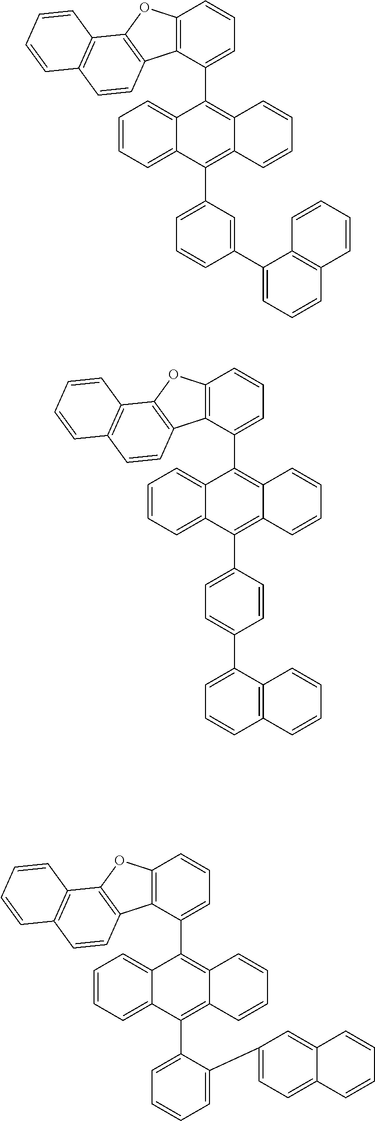 [SMILES: c1cc(-c2cccc3ccccc23)cc(-c2c3ccccc3c(-c3cccc4oc5c6ccccc6ccc5c34)c3ccccc23)c1 ; c1ccc2c(-c3ccc(-c4c5ccccc5c(-c5cccc6oc7c8ccccc8ccc7c56)c5ccccc45)cc3)cccc2c1 ; c1ccc(-c2c3ccccc3c(-c3cccc4oc5c6ccccc6ccc5c34)c3ccccc23)c(-c2ccc3ccccc3c2)c1]

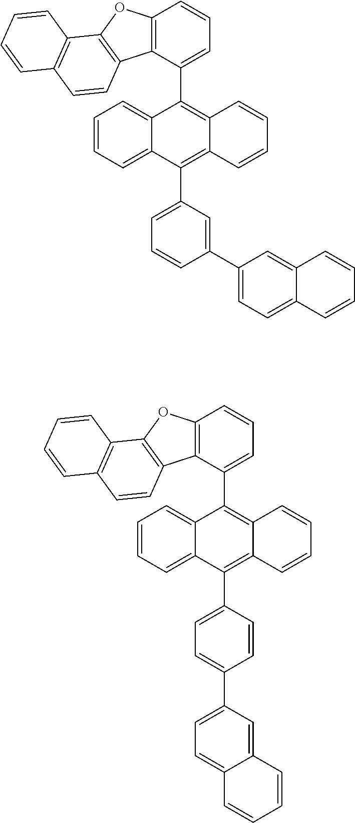 [SMILES: c1cc(-c2ccc3ccccc3c2)cc(-c2c3ccccc3c(-c3cccc4oc5c6ccccc6ccc5c34)c3ccccc23)c1 ; c1ccc2cc(-c3ccc(-c4c5ccccc5c(-c5cccc6oc7c8ccccc8ccc7c56)c5ccccc45)cc3)ccc2c1]

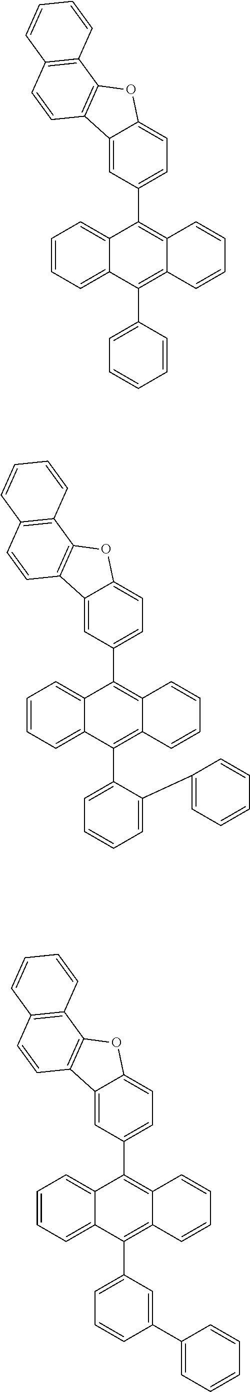 [SMILES: c1ccc(-c2c3ccccc3c(-c3ccc4oc5c6ccccc6ccc5c4c3)c3ccccc23)cc1 ; c1ccc(-c2ccccc2-c2c3ccccc3c(-c3ccc4oc5c6ccccc6ccc5c4c3)c3ccccc23)cc1 ; c1ccc(-c2cccc(-c3c4ccccc4c(-c4ccc5oc6c7ccccc7ccc6c5c4)c4ccccc34)c2)cc1]

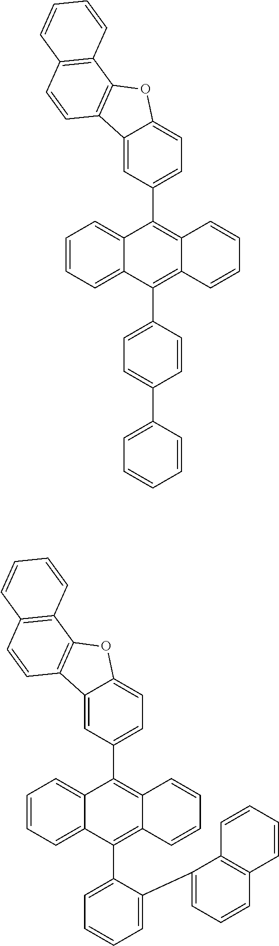 [SMILES: c1ccc(-c2ccc(-c3c4ccccc4c(-c4ccc5oc6c7ccccc7ccc6c5c4)c4ccccc34)cc2)cc1 ; c1ccc(-c2c3ccccc3c(-c3ccc4oc5c6ccccc6ccc5c4c3)c3ccccc23)c(-c2cccc3ccccc23)c1]

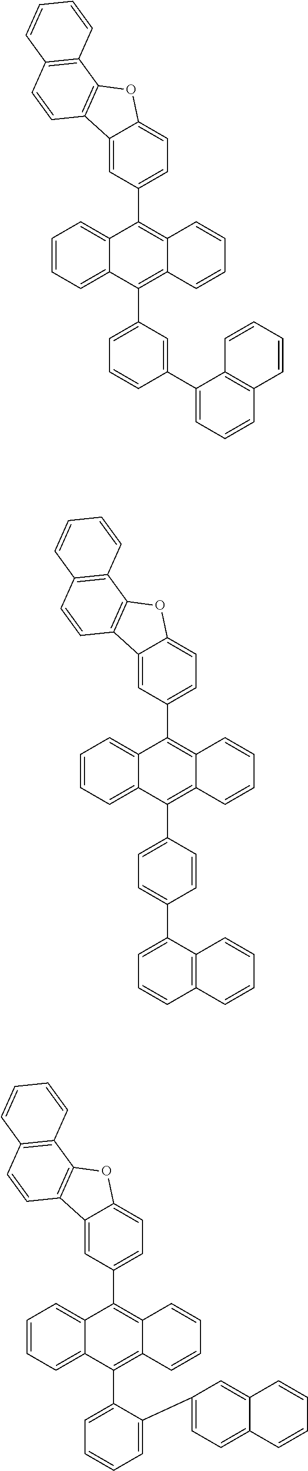 [SMILES: c1cc(-c2cccc3ccccc23)cc(-c2c3ccccc3c(-c3ccc4oc5c6ccccc6ccc5c4c3)c3ccccc23)c1 ; c1ccc2c(-c3ccc(-c4c5ccccc5c(-c5ccc6oc7c8ccccc8ccc7c6c5)c5ccccc45)cc3)cccc2c1 ; c1ccc(-c2c3ccccc3c(-c3ccc4oc5c6ccccc6ccc5c4c3)c3ccccc23)c(-c2ccc3ccccc3c2)c1]

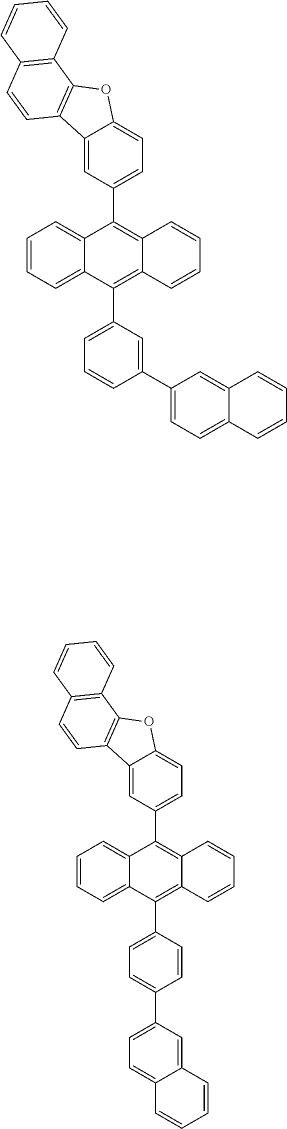 [SMILES: c1cc(-c2ccc3ccccc3c2)cc(-c2c3ccccc3c(-c3ccc4oc5c6ccccc6ccc5c4c3)c3ccccc23)c1 ; c1ccc2cc(-c3ccc(-c4c5ccccc5c(-c5ccc6oc7c8ccccc8ccc7c6c5)c5ccccc45)cc3)ccc2c1]

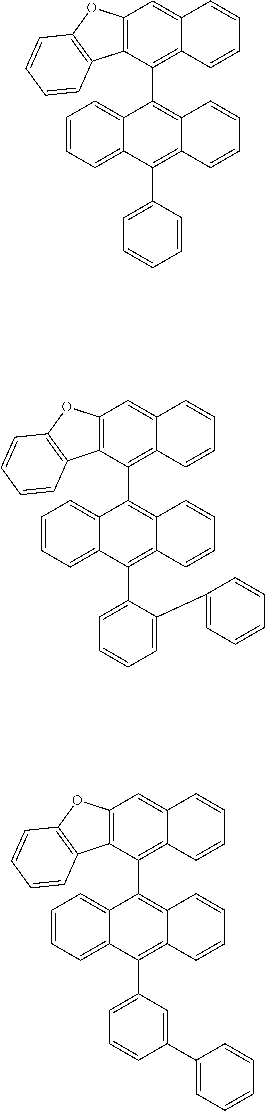 [SMILES: c1ccc(-c2c3ccccc3c(-c3c4ccccc4cc4oc5ccccc5c34)c3ccccc23)cc1 ; c1ccc(-c2ccccc2-c2c3ccccc3c(-c3c4ccccc4cc4oc5ccccc5c34)c3ccccc23)cc1 ; c1ccc(-c2cccc(-c3c4ccccc4c(-c4c5ccccc5cc5oc6ccccc6c45)c4ccccc34)c2)cc1]

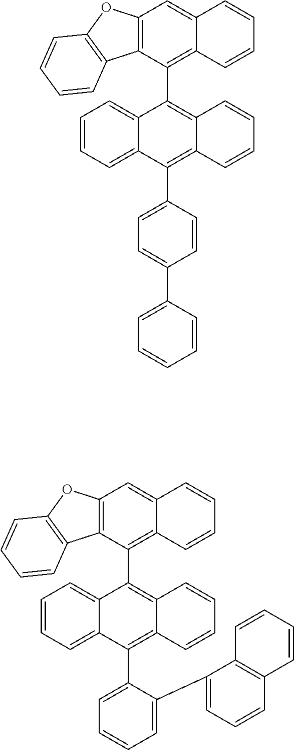 [SMILES: c1ccc(-c2ccc(-c3c4ccccc4c(-c4c5ccccc5cc5oc6ccccc6c45)c4ccccc34)cc2)cc1 ; c1ccc(-c2c3ccccc3c(-c3c4ccccc4cc4oc5ccccc5c34)c3ccccc23)c(-c2cccc3ccccc23)c1]

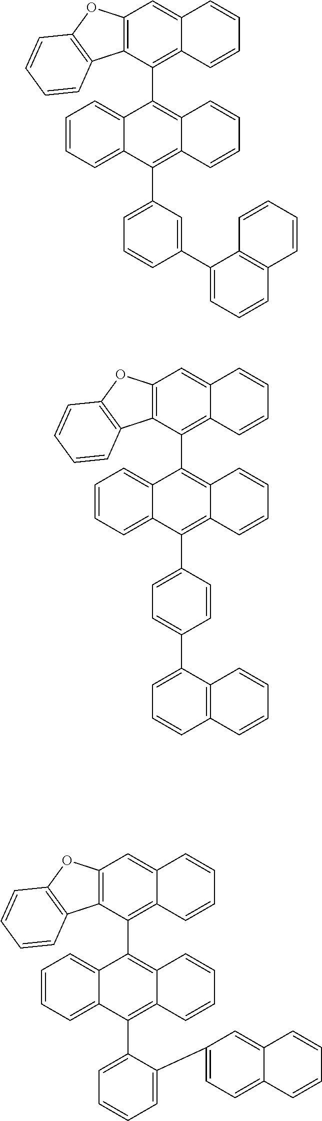 [SMILES: c1cc(-c2cccc3ccccc23)cc(-c2c3ccccc3c(-c3c4ccccc4cc4oc5ccccc5c34)c3ccccc23)c1 ; c1ccc2c(-c3ccc(-c4c5ccccc5c(-c5c6ccccc6cc6oc7ccccc7c56)c5ccccc45)cc3)cccc2c1 ; c1ccc(-c2c3ccccc3c(-c3c4ccccc4cc4oc5ccccc5c34)c3ccccc23)c(-c2ccc3ccccc3c2)c1]

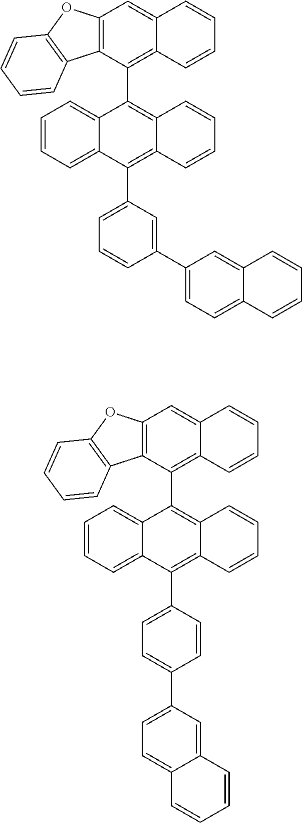 [SMILES: c1cc(-c2ccc3ccccc3c2)cc(-c2c3ccccc3c(-c3c4ccccc4cc4oc5ccccc5c34)c3ccccc23)c1 ; c1ccc2cc(-c3ccc(-c4c5ccccc5c(-c5c6ccccc6cc6oc7ccccc7c56)c5ccccc45)cc3)ccc2c1]

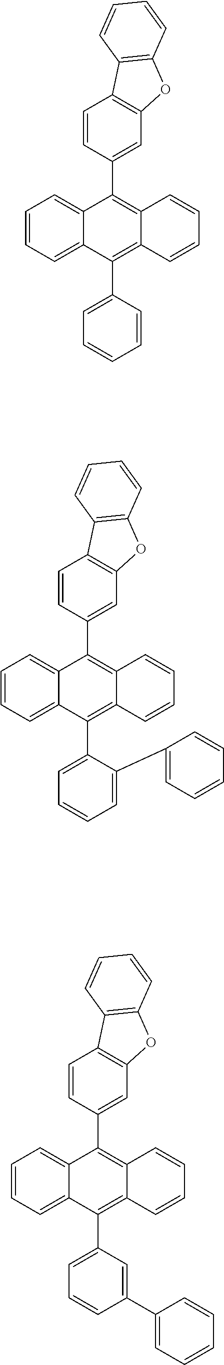 [SMILES: c1ccc(-c2c3ccccc3c(-c3ccc4c(c3)oc3ccccc34)c3ccccc23)cc1 ; c1ccc(-c2ccccc2-c2c3ccccc3c(-c3ccc4c(c3)oc3ccccc34)c3ccccc23)cc1 ; c1ccc(-c2cccc(-c3c4ccccc4c(-c4ccc5c(c4)oc4ccccc45)c4ccccc34)c2)cc1]

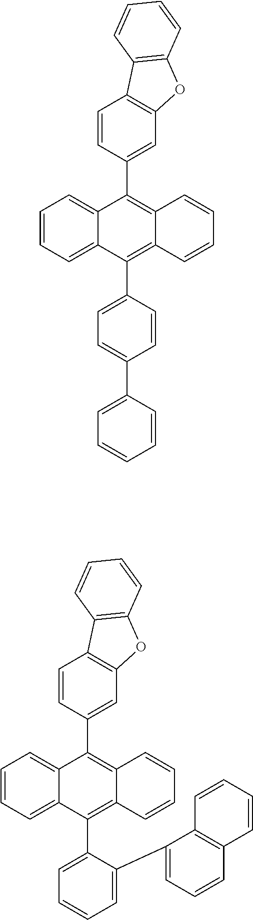 [SMILES: c1ccc(-c2ccc(-c3c4ccccc4c(-c4ccc5c(c4)oc4ccccc45)c4ccccc34)cc2)cc1 ; c1ccc(-c2c3ccccc3c(-c3ccc4c(c3)oc3ccccc34)c3ccccc23)c(-c2cccc3ccccc23)c1]

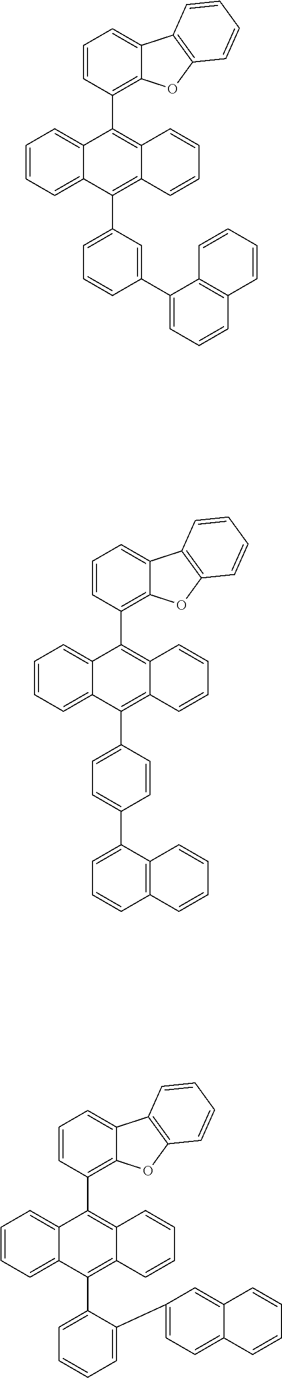 [SMILES: c1cc(-c2cccc3ccccc23)cc(-c2c3ccccc3c(-c3cccc4c3oc3ccccc34)c3ccccc23)c1 ; c1ccc2c(-c3ccc(-c4c5ccccc5c(-c5cccc6c5oc5ccccc56)c5ccccc45)cc3)cccc2c1 ; c1ccc(-c2c3ccccc3c(-c3cccc4c3oc3ccccc34)c3ccccc23)c(-c2ccc3ccccc3c2)c1]

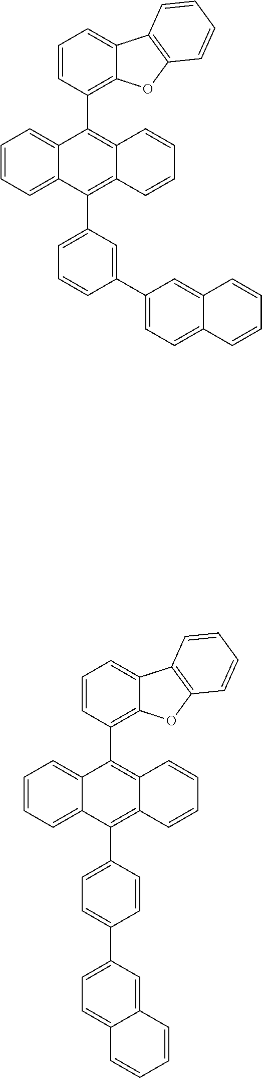 [SMILES: c1cc(-c2ccc3ccccc3c2)cc(-c2c3ccccc3c(-c3cccc4c3oc3ccccc34)c3ccccc23)c1 ; c1ccc2cc(-c3ccc(-c4c5ccccc5c(-c5cccc6c5oc5ccccc56)c5ccccc45)cc3)ccc2c1]

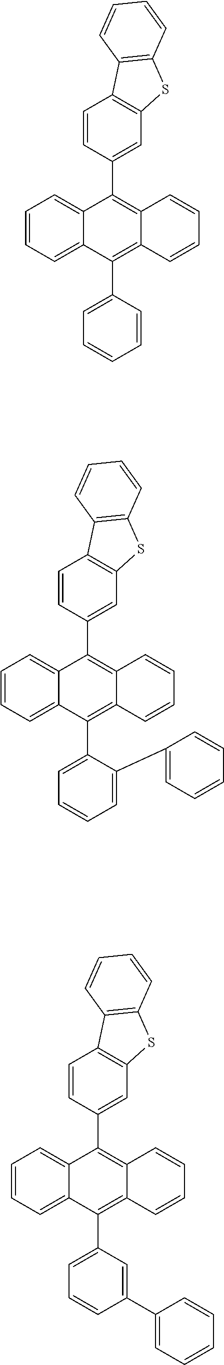 [SMILES: c1ccc(-c2c3ccccc3c(-c3ccc4c(c3)sc3ccccc34)c3ccccc23)cc1 ; c1ccc(-c2ccccc2-c2c3ccccc3c(-c3ccc4c(c3)sc3ccccc34)c3ccccc23)cc1 ; c1ccc(-c2cccc(-c3c4ccccc4c(-c4ccc5c(c4)sc4ccccc45)c4ccccc34)c2)cc1]

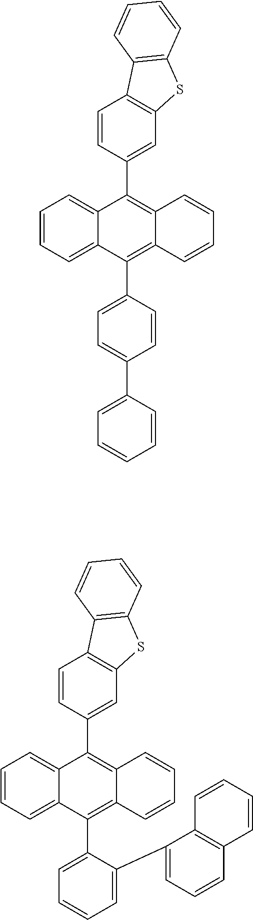 [SMILES: c1ccc(-c2ccc(-c3c4ccccc4c(-c4ccc5c(c4)sc4ccccc45)c4ccccc34)cc2)cc1 ; c1ccc(-c2c3ccccc3c(-c3ccc4c(c3)sc3ccccc34)c3ccccc23)c(-c2cccc3ccccc23)c1]

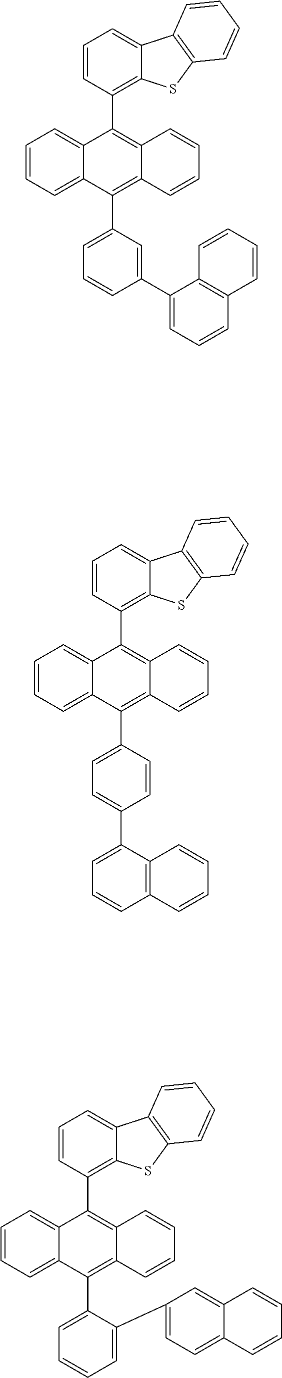 [SMILES: c1cc(-c2cccc3ccccc23)cc(-c2c3ccccc3c(-c3cccc4c3sc3ccccc34)c3ccccc23)c1 ; c1ccc2c(-c3ccc(-c4c5ccccc5c(-c5cccc6c5sc5ccccc56)c5ccccc45)cc3)cccc2c1 ; c1ccc(-c2c3ccccc3c(-c3cccc4c3sc3ccccc34)c3ccccc23)c(-c2ccc3ccccc3c2)c1]

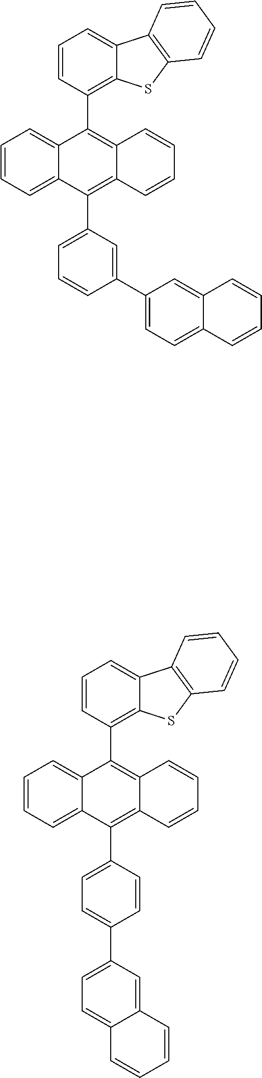 [SMILES: c1cc(-c2ccc3ccccc3c2)cc(-c2c3ccccc3c(-c3cccc4c3sc3ccccc34)c3ccccc23)c1 ; c1ccc2cc(-c3ccc(-c4c5ccccc5c(-c5cccc6c5sc5ccccc56)c5ccccc45)cc3)ccc2c1]

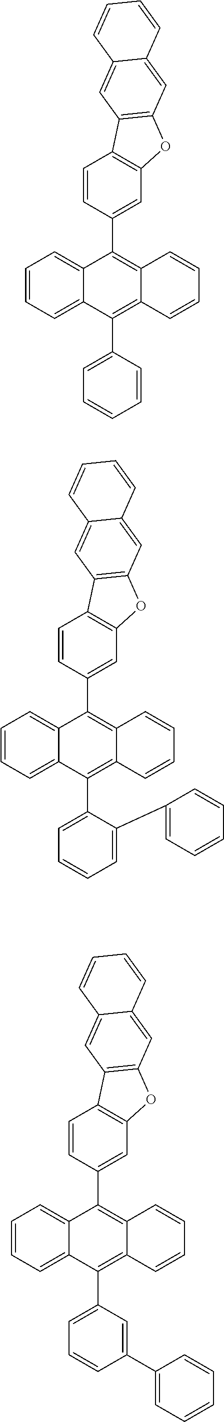

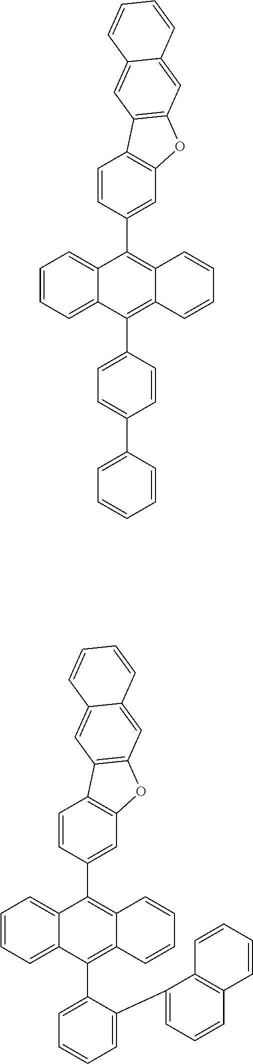 [SMILES: c1ccc(-c2ccc(-c3c4ccccc4c(-c4ccc5c(c4)oc4cc6ccccc6cc45)c4ccccc34)cc2)cc1 ; c1ccc(-c2c3ccccc3c(-c3ccc4c(c3)oc3cc5ccccc5cc34)c3ccccc23)c(-c2cccc3ccccc23)c1]

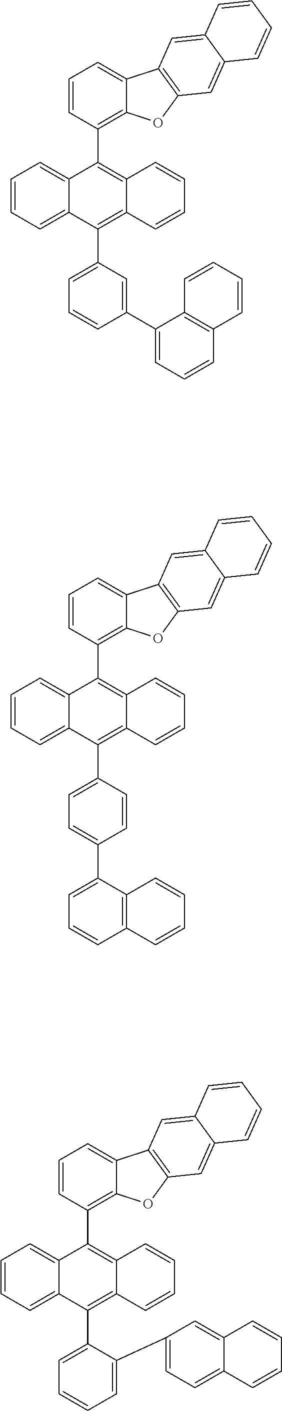 [SMILES: c1cc(-c2cccc3ccccc23)cc(-c2c3ccccc3c(-c3cccc4c3oc3cc5ccccc5cc34)c3ccccc23)c1 ; c1ccc2cc3c(cc2c1)oc1c(-c2c4ccccc4c(-c4ccc(-c5cccc6ccccc56)cc4)c4ccccc24)cccc13 ; c1ccc(-c2c3ccccc3c(-c3cccc4c3oc3cc5ccccc5cc34)c3ccccc23)c(-c2ccc3ccccc3c2)c1]

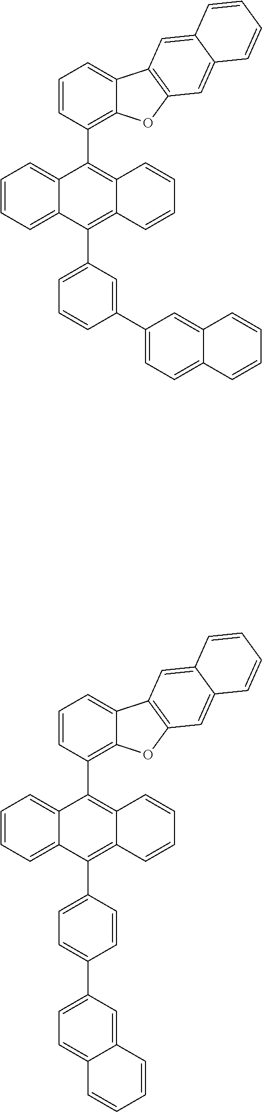 [SMILES: c1cc(-c2ccc3ccccc3c2)cc(-c2c3ccccc3c(-c3cccc4c3oc3cc5ccccc5cc34)c3ccccc23)c1 ; c1ccc2cc(-c3ccc(-c4c5ccccc5c(-c5cccc6c5oc5cc7ccccc7cc56)c5ccccc45)cc3)ccc2c1]

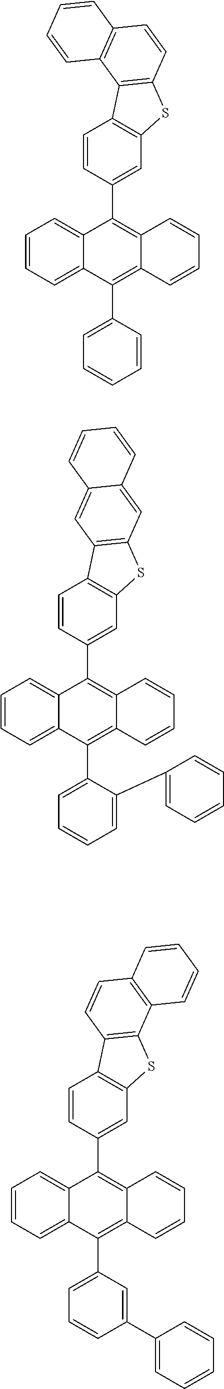

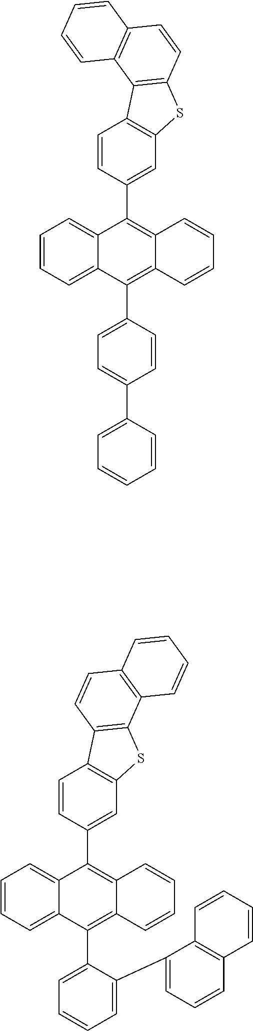 [SMILES: c1ccc(-c2ccc(-c3c4ccccc4c(-c4ccc5c(c4)sc4ccc6ccccc6c45)c4ccccc34)cc2)cc1 ; c1ccc(-c2c3ccccc3c(-c3ccc4c(c3)sc3c5ccccc5ccc43)c3ccccc23)c(-c2cccc3ccccc23)c1]

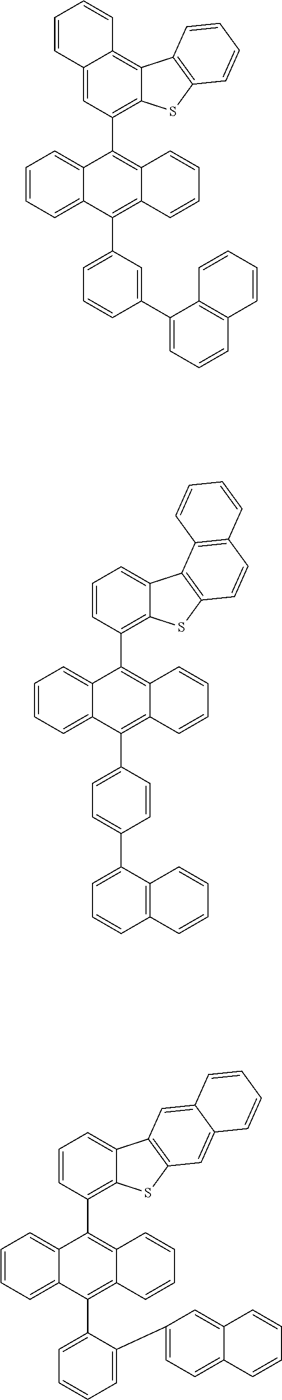 [SMILES: c1cc(-c2cccc3ccccc23)cc(-c2c3ccccc3c(-c3cc4ccccc4c4c3sc3ccccc34)c3ccccc23)c1 ; c1ccc2c(-c3ccc(-c4c5ccccc5c(-c5cccc6c5sc5ccc7ccccc7c56)c5ccccc45)cc3)cccc2c1 ; c1ccc(-c2c3ccccc3c(-c3cccc4c3sc3cc5ccccc5cc34)c3ccccc23)c(-c2ccc3ccccc3c2)c1]

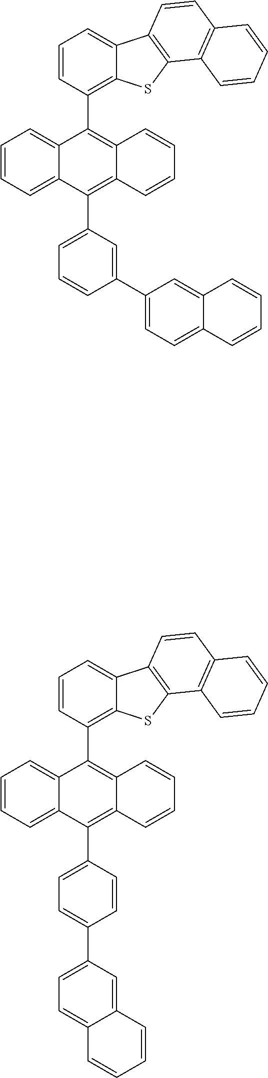 [SMILES: c1cc(-c2ccc3ccccc3c2)cc(-c2c3ccccc3c(-c3cccc4c3sc3c5ccccc5ccc43)c3ccccc23)c1 ; c1ccc2cc(-c3ccc(-c4c5ccccc5c(-c5cccc6c5sc5c7ccccc7ccc65)c5ccccc45)cc3)ccc2c1]

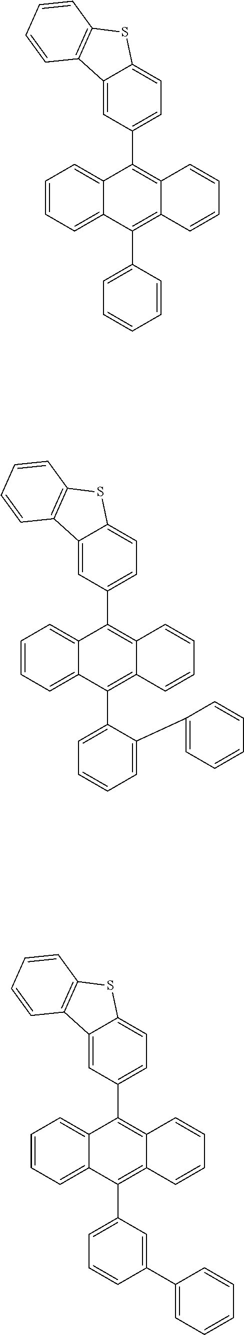 [SMILES: c1ccc(-c2c3ccccc3c(-c3ccc4sc5ccccc5c4c3)c3ccccc23)cc1 ; c1ccc(-c2ccccc2-c2c3ccccc3c(-c3ccc4sc5ccccc5c4c3)c3ccccc23)cc1 ; c1ccc(-c2cccc(-c3c4ccccc4c(-c4ccc5sc6ccccc6c5c4)c4ccccc34)c2)cc1]

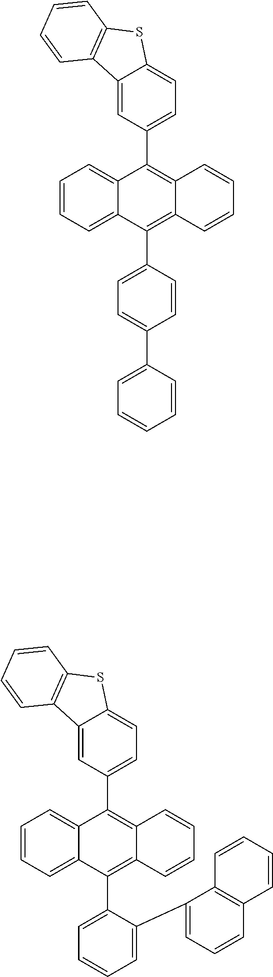 [SMILES: c1ccc(-c2ccc(-c3c4ccccc4c(-c4ccc5sc6ccccc6c5c4)c4ccccc34)cc2)cc1 ; c1ccc(-c2c3ccccc3c(-c3ccc4sc5ccccc5c4c3)c3ccccc23)c(-c2cccc3ccccc23)c1]

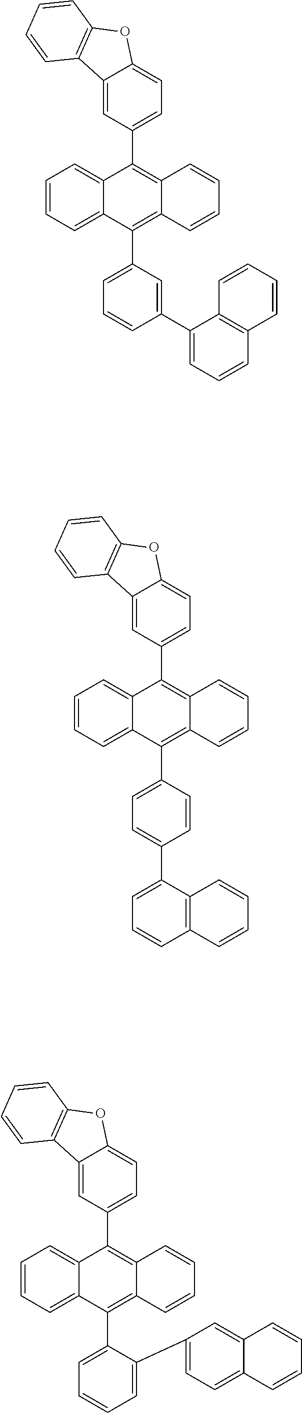 [SMILES: c1cc(-c2cccc3ccccc23)cc(-c2c3ccccc3c(-c3ccc4oc5ccccc5c4c3)c3ccccc23)c1 ; c1ccc2c(-c3ccc(-c4c5ccccc5c(-c5ccc6oc7ccccc7c6c5)c5ccccc45)cc3)cccc2c1 ; c1ccc(-c2c3ccccc3c(-c3ccc4oc5ccccc5c4c3)c3ccccc23)c(-c2ccc3ccccc3c2)c1]

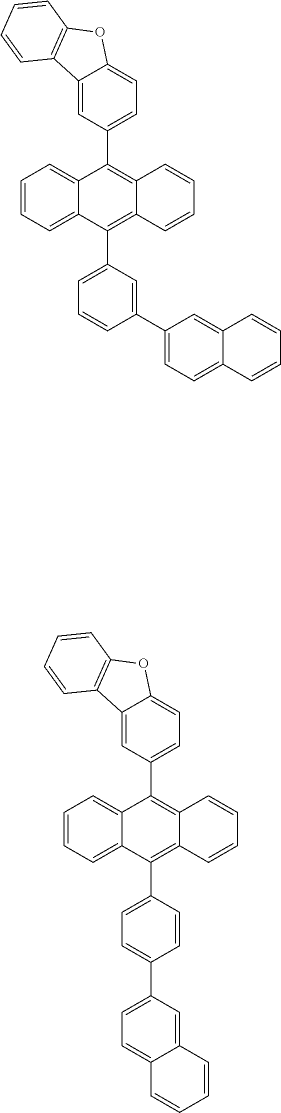 [SMILES: c1cc(-c2ccc3ccccc3c2)cc(-c2c3ccccc3c(-c3ccc4oc5ccccc5c4c3)c3ccccc23)c1 ; c1ccc2cc(-c3ccc(-c4c5ccccc5c(-c5ccc6oc7ccccc7c6c5)c5ccccc45)cc3)ccc2c1]

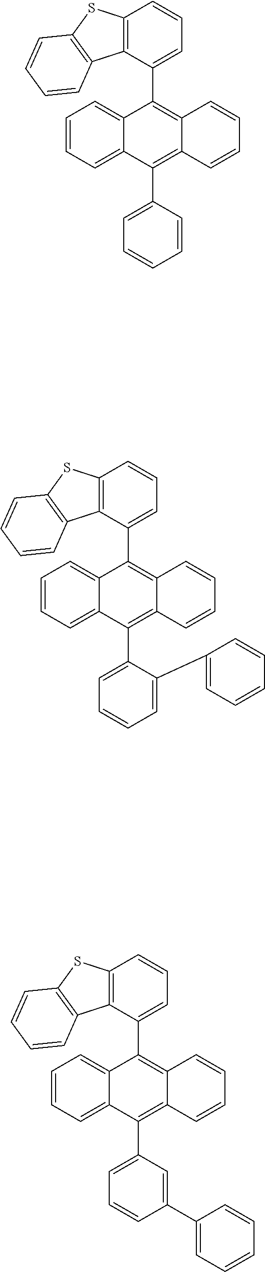 [SMILES: c1ccc(-c2c3ccccc3c(-c3cccc4sc5ccccc5c34)c3ccccc23)cc1 ; c1ccc(-c2ccccc2-c2c3ccccc3c(-c3cccc4sc5ccccc5c34)c3ccccc23)cc1 ; c1ccc(-c2cccc(-c3c4ccccc4c(-c4cccc5sc6ccccc6c45)c4ccccc34)c2)cc1]

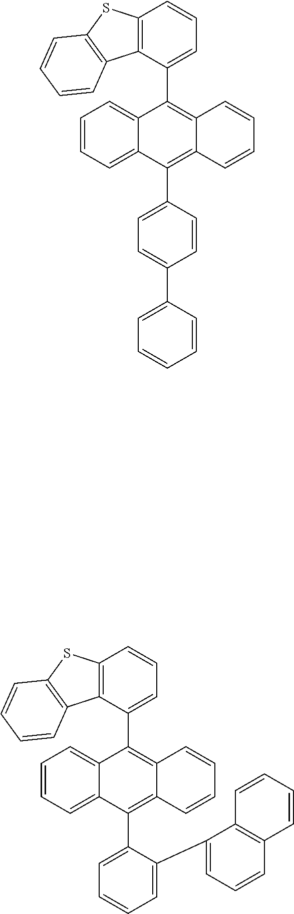 [SMILES: c1ccc(-c2ccc(-c3c4ccccc4c(-c4cccc5sc6ccccc6c45)c4ccccc34)cc2)cc1 ; c1ccc(-c2c3ccccc3c(-c3cccc4sc5ccccc5c34)c3ccccc23)c(-c2cccc3ccccc23)c1]

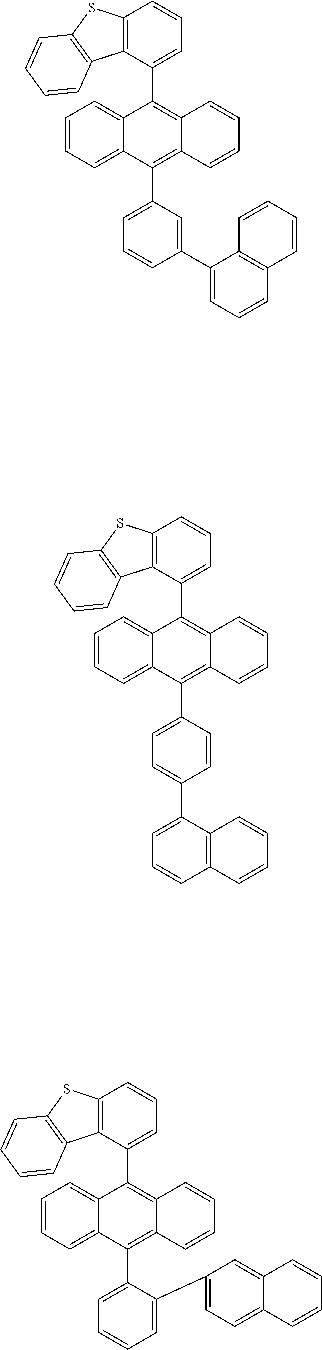 [SMILES: c1cc(-c2cccc3ccccc23)cc(-c2c3ccccc3c(-c3cccc4sc5ccccc5c34)c3ccccc23)c1 ; c1ccc2c(-c3ccc(-c4c5ccccc5c(-c5cccc6sc7ccccc7c56)c5ccccc45)cc3)cccc2c1 ; c1ccc(-c2c3ccccc3c(-c3cccc4sc5ccccc5c34)c3ccccc23)c(-c2ccc3ccccc3c2)c1]

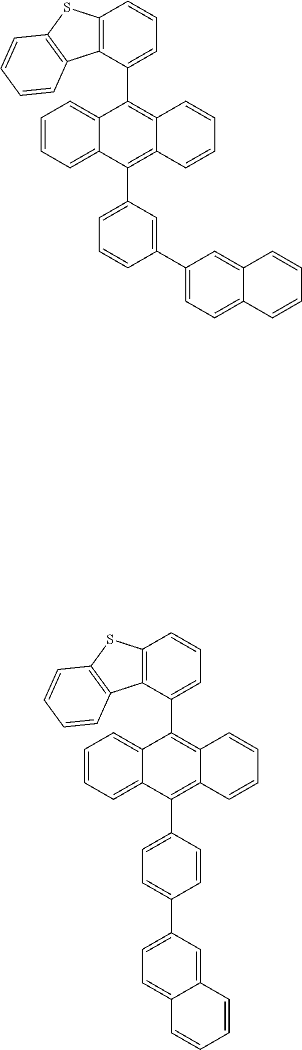 [SMILES: c1cc(-c2ccc3ccccc3c2)cc(-c2c3ccccc3c(-c3cccc4sc5ccccc5c34)c3ccccc23)c1 ; c1ccc2cc(-c3ccc(-c4c5ccccc5c(-c5cccc6sc7ccccc7c56)c5ccccc45)cc3)ccc2c1]

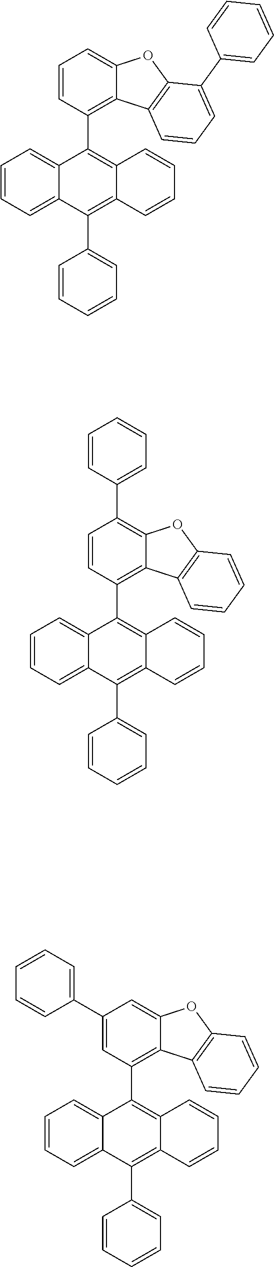 [SMILES: c1ccc(-c2c3ccccc3c(-c3cccc4oc5c(-c6ccccc6)cccc5c34)c3ccccc23)cc1 ; c1ccc(-c2c3ccccc3c(-c3ccc(-c4ccccc4)c4oc5ccccc5c34)c3ccccc23)cc1 ; c1ccc(-c2cc(-c3c4ccccc4c(-c4ccccc4)c4ccccc34)c3c(c2)oc2ccccc23)cc1]

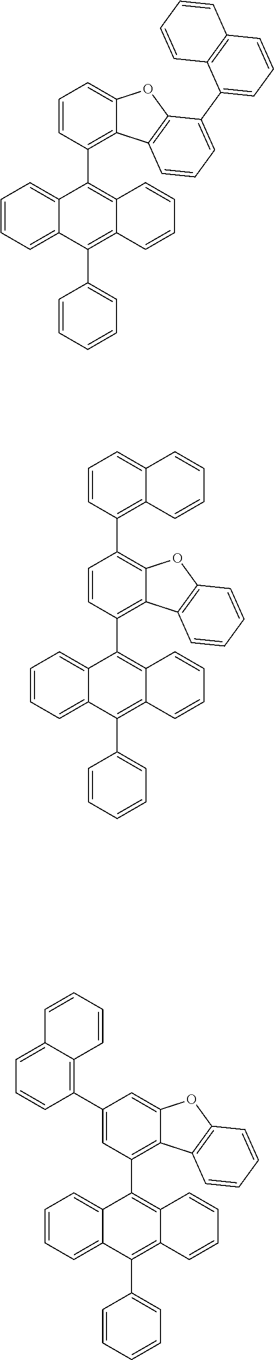 [SMILES: c1ccc(-c2c3ccccc3c(-c3cccc4oc5c(-c6cccc7ccccc67)cccc5c34)c3ccccc23)cc1 ; c1ccc(-c2c3ccccc3c(-c3ccc(-c4cccc5ccccc45)c4oc5ccccc5c34)c3ccccc23)cc1 ; c1ccc(-c2c3ccccc3c(-c3cc(-c4cccc5ccccc45)cc4oc5ccccc5c34)c3ccccc23)cc1]

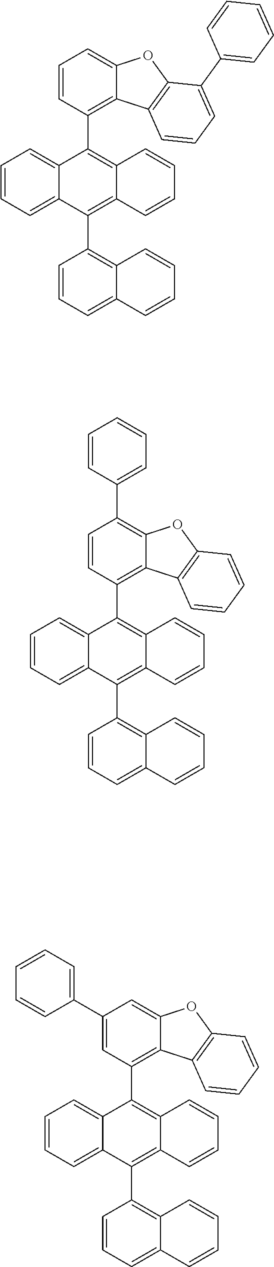 [SMILES: c1ccc(-c2cccc3c2oc2cccc(-c4c5ccccc5c(-c5cccc6ccccc56)c5ccccc45)c23)cc1 ; c1ccc(-c2ccc(-c3c4ccccc4c(-c4cccc5ccccc45)c4ccccc34)c3c2oc2ccccc23)cc1 ; c1ccc(-c2cc(-c3c4ccccc4c(-c4cccc5ccccc45)c4ccccc34)c3c(c2)oc2ccccc23)cc1]

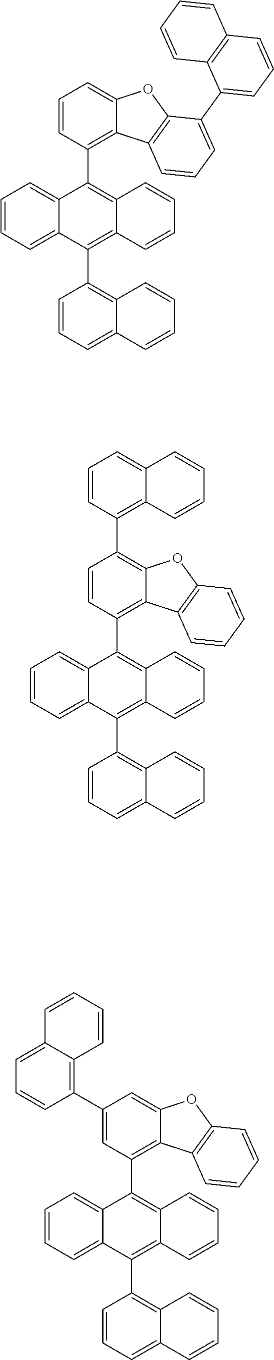 [SMILES: c1ccc2c(-c3c4ccccc4c(-c4cccc5oc6c(-c7cccc8ccccc78)cccc6c45)c4ccccc34)cccc2c1 ; c1ccc2c(-c3c4ccccc4c(-c4ccc(-c5cccc6ccccc56)c5oc6ccccc6c45)c4ccccc34)cccc2c1 ; c1ccc2c(-c3cc(-c4c5ccccc5c(-c5cccc6ccccc56)c5ccccc45)c4c(c3)oc3ccccc34)cccc2c1]

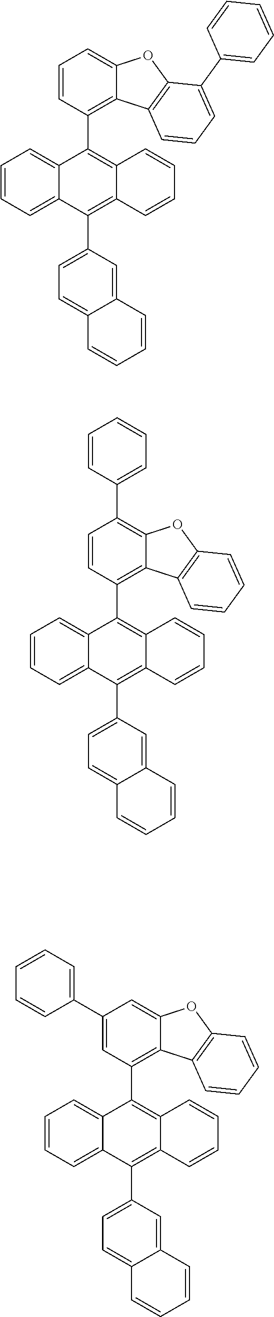 [SMILES: c1ccc(-c2cccc3c2oc2cccc(-c4c5ccccc5c(-c5ccc6ccccc6c5)c5ccccc45)c23)cc1 ; c1ccc(-c2ccc(-c3c4ccccc4c(-c4ccc5ccccc5c4)c4ccccc34)c3c2oc2ccccc23)cc1 ; c1ccc(-c2cc(-c3c4ccccc4c(-c4ccc5ccccc5c4)c4ccccc34)c3c(c2)oc2ccccc23)cc1]

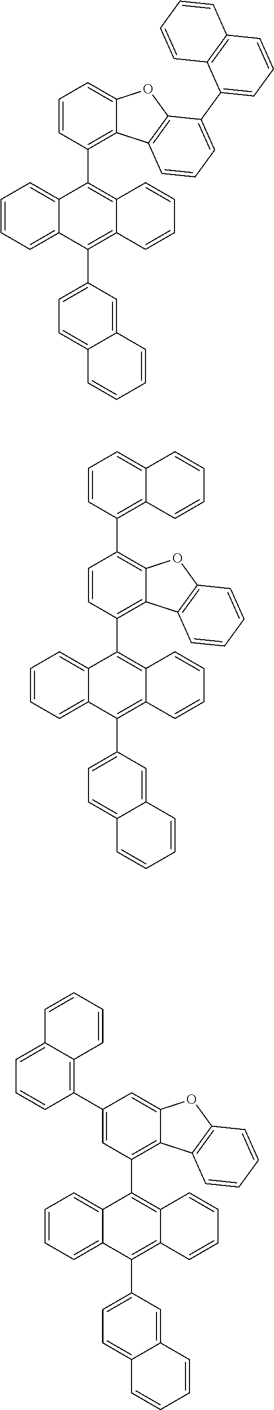 [SMILES: c1ccc2cc(-c3c4ccccc4c(-c4cccc5oc6c(-c7cccc8ccccc78)cccc6c45)c4ccccc34)ccc2c1 ; c1ccc2cc(-c3c4ccccc4c(-c4ccc(-c5cccc6ccccc56)c5oc6ccccc6c45)c4ccccc34)ccc2c1 ; c1ccc2cc(-c3c4ccccc4c(-c4cc(-c5cccc6ccccc56)cc5oc6ccccc6c45)c4ccccc34)ccc2c1]

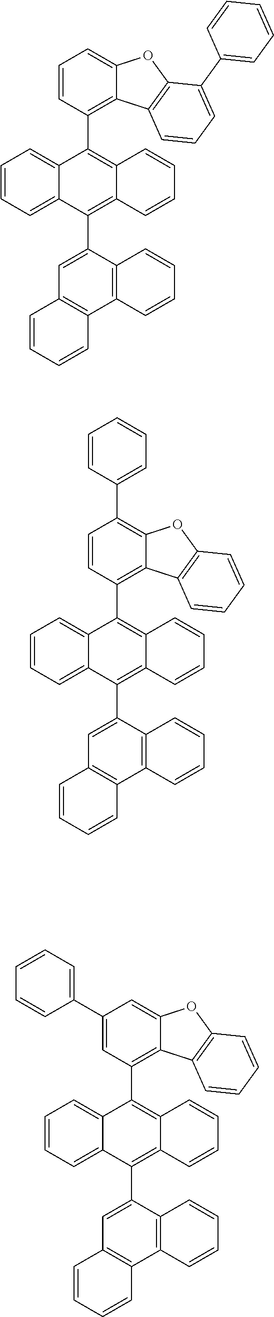 [SMILES: c1ccc(-c2cccc3c2oc2cccc(-c4c5ccccc5c(-c5cc6ccccc6c6ccccc56)c5ccccc45)c23)cc1 ; c1ccc(-c2ccc(-c3c4ccccc4c(-c4cc5ccccc5c5ccccc45)c4ccccc34)c3c2oc2ccccc23)cc1 ; c1ccc(-c2cc(-c3c4ccccc4c(-c4cc5ccccc5c5ccccc45)c4ccccc34)c3c(c2)oc2ccccc23)cc1]

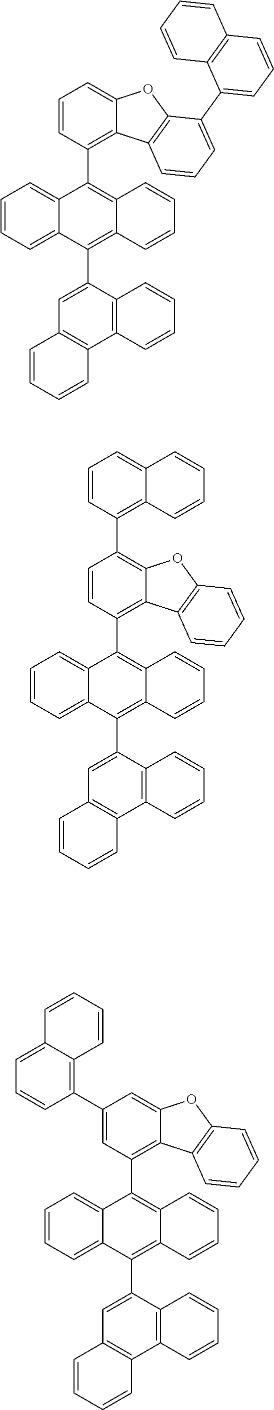 [SMILES: c1ccc2c(-c3cccc4c3oc3cccc(-c5c6ccccc6c(-c6cc7ccccc7c7ccccc67)c6ccccc56)c34)cccc2c1 ; c1ccc2c(-c3ccc(-c4c5ccccc5c(-c5cc6ccccc6c6ccccc56)c5ccccc45)c4c3oc3ccccc34)cccc2c1 ; c1ccc2c(-c3cc(-c4c5ccccc5c(-c5cc6ccccc6c6ccccc56)c5ccccc45)c4c(c3)oc3ccccc34)cccc2c1]

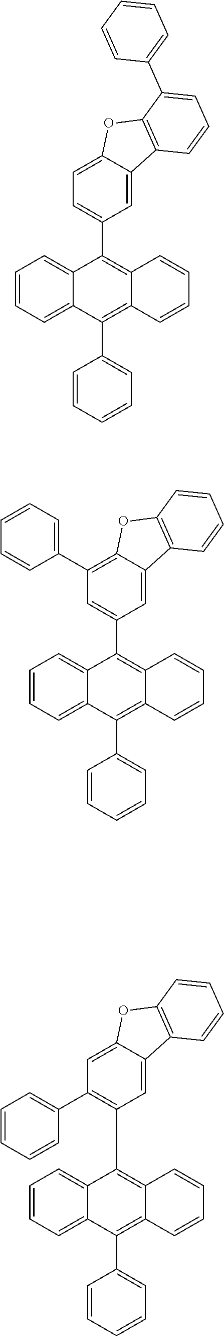 [SMILES: c1ccc(-c2c3ccccc3c(-c3ccc4oc5c(-c6ccccc6)cccc5c4c3)c3ccccc23)cc1 ; c1ccc(-c2c3ccccc3c(-c3cc(-c4ccccc4)c4oc5ccccc5c4c3)c3ccccc23)cc1 ; c1ccc(-c2cc3oc4ccccc4c3cc2-c2c3ccccc3c(-c3ccccc3)c3ccccc23)cc1]

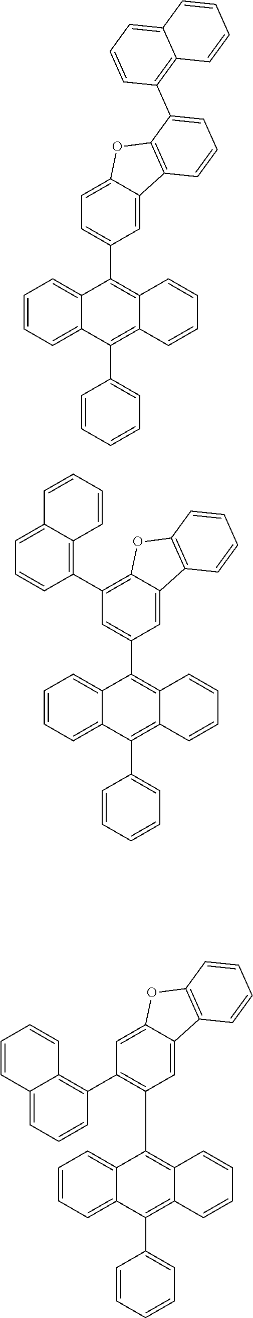

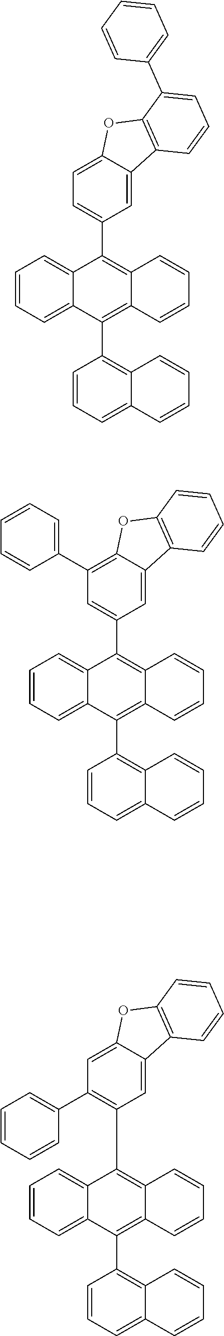 [SMILES: c1ccc(-c2cccc3c2oc2ccc(-c4c5ccccc5c(-c5cccc6ccccc56)c5ccccc45)cc23)cc1 ; c1ccc(-c2cc(-c3c4ccccc4c(-c4cccc5ccccc45)c4ccccc34)cc3c2oc2ccccc23)cc1 ; c1ccc(-c2cc3oc4ccccc4c3cc2-c2c3ccccc3c(-c3cccc4ccccc34)c3ccccc23)cc1]

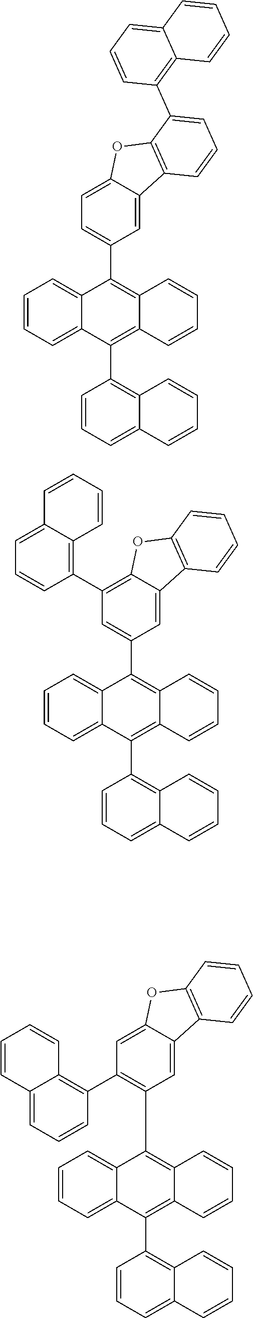

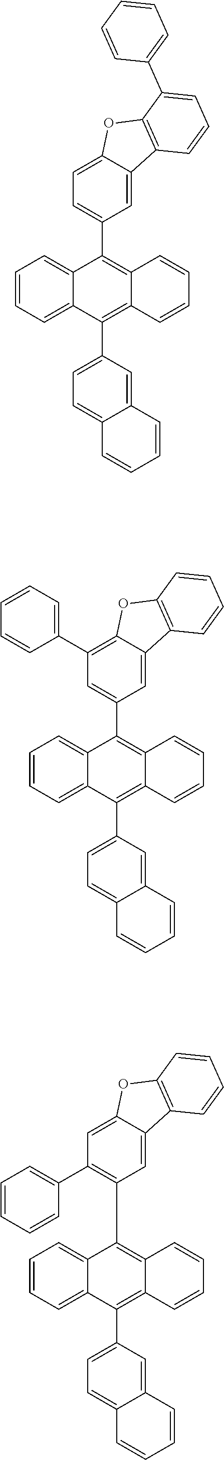 [SMILES: c1ccc(-c2cccc3c2oc2ccc(-c4c5ccccc5c(-c5ccc6ccccc6c5)c5ccccc45)cc23)cc1 ; c1ccc(-c2cc(-c3c4ccccc4c(-c4ccc5ccccc5c4)c4ccccc34)cc3c2oc2ccccc23)cc1 ; c1ccc(-c2cc3oc4ccccc4c3cc2-c2c3ccccc3c(-c3ccc4ccccc4c3)c3ccccc23)cc1]

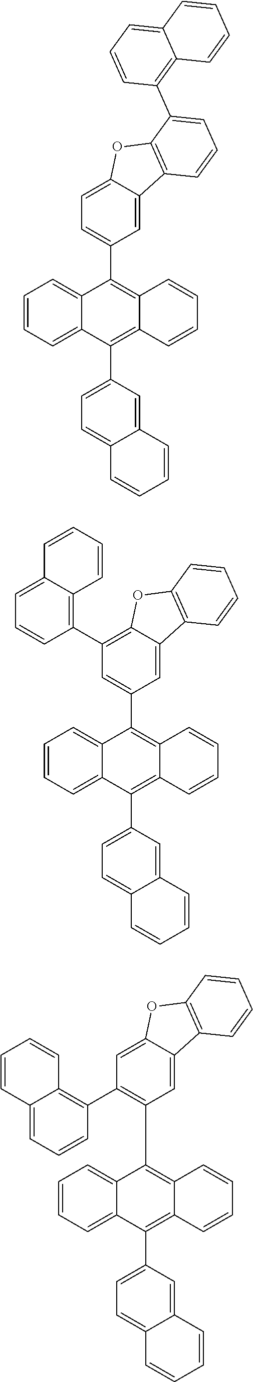 [SMILES: c1ccc2cc(-c3c4ccccc4c(-c4ccc5oc6c(-c7cccc8ccccc78)cccc6c5c4)c4ccccc34)ccc2c1 ; c1ccc2cc(-c3c4ccccc4c(-c4cc(-c5cccc6ccccc56)c5oc6ccccc6c5c4)c4ccccc34)ccc2c1 ; c1ccc2cc(-c3c4ccccc4c(-c4cc5c(cc4-c4cccc6ccccc46)oc4ccccc45)c4ccccc34)ccc2c1]

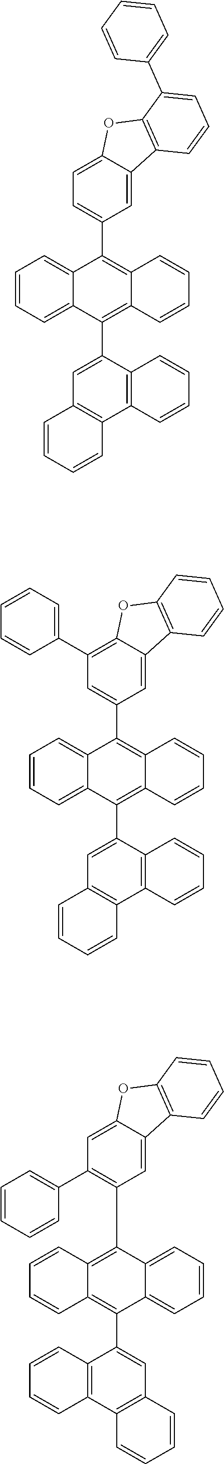 [SMILES: c1ccc(-c2cccc3c2oc2ccc(-c4c5ccccc5c(-c5cc6ccccc6c6ccccc56)c5ccccc45)cc23)cc1 ; c1ccc(-c2cc(-c3c4ccccc4c(-c4cc5ccccc5c5ccccc45)c4ccccc34)cc3c2oc2ccccc23)cc1 ; c1ccc(-c2cc3oc4ccccc4c3cc2-c2c3ccccc3c(-c3cc4ccccc4c4ccccc34)c3ccccc23)cc1]

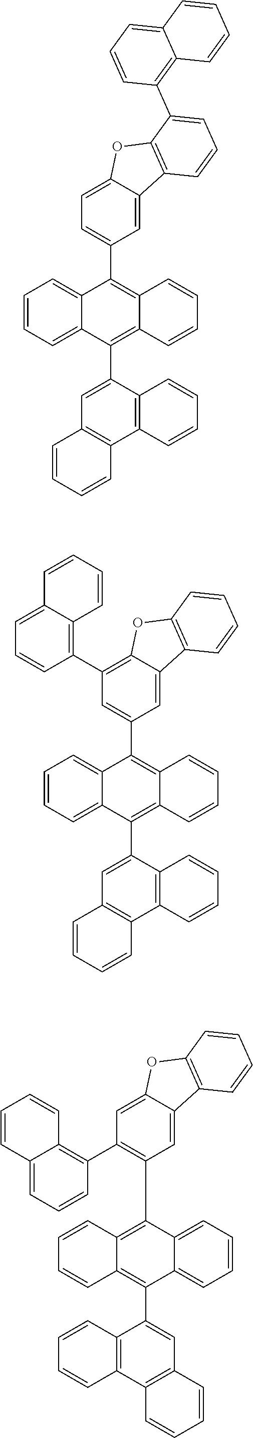 [SMILES: c1ccc2c(-c3cccc4c3oc3ccc(-c5c6ccccc6c(-c6cc7ccccc7c7ccccc67)c6ccccc56)cc34)cccc2c1 ; c1ccc2c(-c3cc(-c4c5ccccc5c(-c5cc6ccccc6c6ccccc56)c5ccccc45)cc4c3oc3ccccc34)cccc2c1 ; c1ccc2c(-c3cc4oc5ccccc5c4cc3-c3c4ccccc4c(-c4cc5ccccc5c5ccccc45)c4ccccc34)cccc2c1]

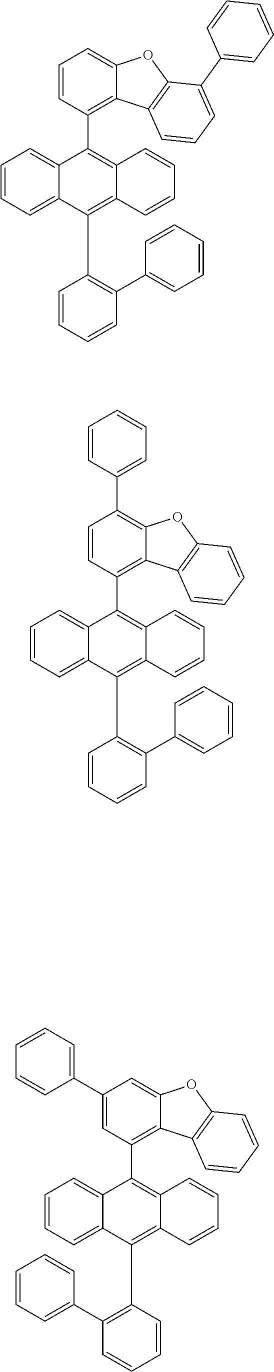 [SMILES: c1ccc(-c2ccccc2-c2c3ccccc3c(-c3cccc4oc5c(-c6ccccc6)cccc5c34)c3ccccc23)cc1 ; c1ccc(-c2ccccc2-c2c3ccccc3c(-c3ccc(-c4ccccc4)c4oc5ccccc5c34)c3ccccc23)cc1 ; c1ccc(-c2cc(-c3c4ccccc4c(-c4ccccc4-c4ccccc4)c4ccccc34)c3c(c2)oc2ccccc23)cc1]

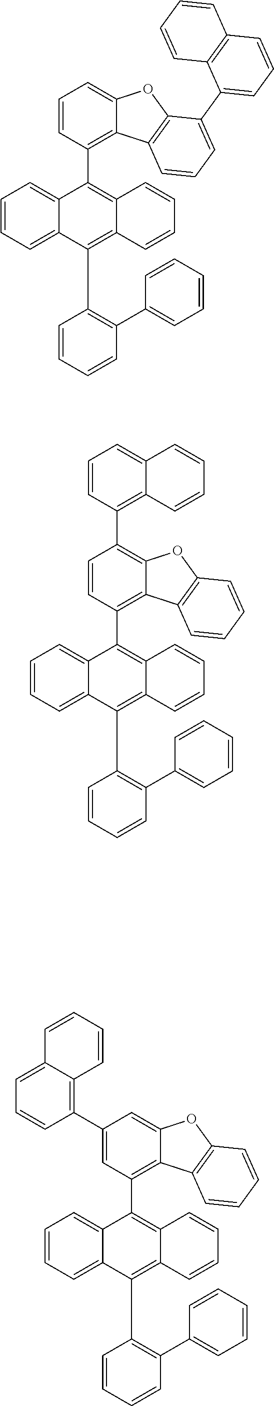 [SMILES: c1ccc(-c2ccccc2-c2c3ccccc3c(-c3cccc4oc5c(-c6cccc7ccccc67)cccc5c34)c3ccccc23)cc1 ; c1ccc(-c2ccccc2-c2c3ccccc3c(-c3ccc(-c4cccc5ccccc45)c4oc5ccccc5c34)c3ccccc23)cc1 ; c1ccc(-c2ccccc2-c2c3ccccc3c(-c3cc(-c4cccc5ccccc45)cc4oc5ccccc5c34)c3ccccc23)cc1]

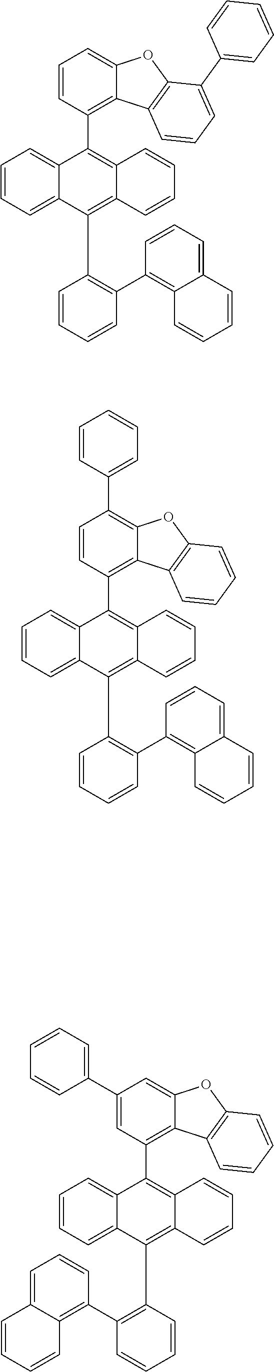 [SMILES: c1ccc(-c2cccc3c2oc2cccc(-c4c5ccccc5c(-c5ccccc5-c5cccc6ccccc56)c5ccccc45)c23)cc1 ; c1ccc(-c2ccc(-c3c4ccccc4c(-c4ccccc4-c4cccc5ccccc45)c4ccccc34)c3c2oc2ccccc23)cc1 ; c1ccc(-c2cc(-c3c4ccccc4c(-c4ccccc4-c4cccc5ccccc45)c4ccccc34)c3c(c2)oc2ccccc23)cc1]

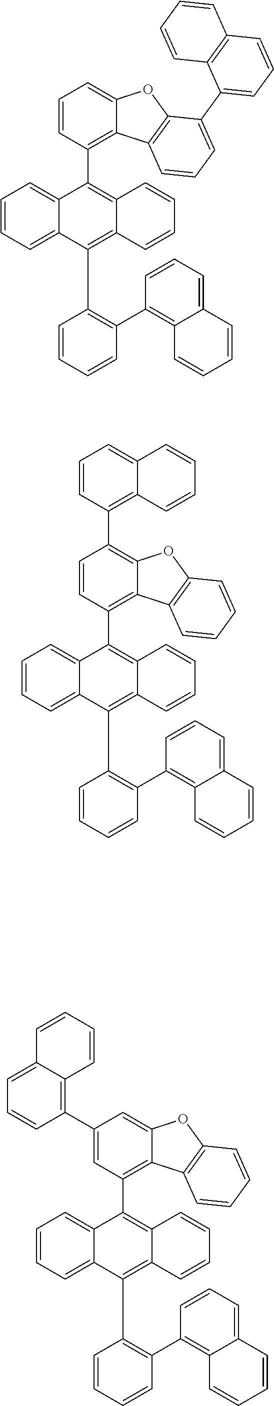 [SMILES: c1ccc(-c2c3ccccc3c(-c3cccc4oc5c(-c6cccc7ccccc67)cccc5c34)c3ccccc23)c(-c2cccc3ccccc23)c1 ; c1ccc(-c2c3ccccc3c(-c3ccc(-c4cccc5ccccc45)c4oc5ccccc5c34)c3ccccc23)c(-c2cccc3ccccc23)c1 ; c1ccc(-c2c3ccccc3c(-c3cc(-c4cccc5ccccc45)cc4oc5ccccc5c34)c3ccccc23)c(-c2cccc3ccccc23)c1]

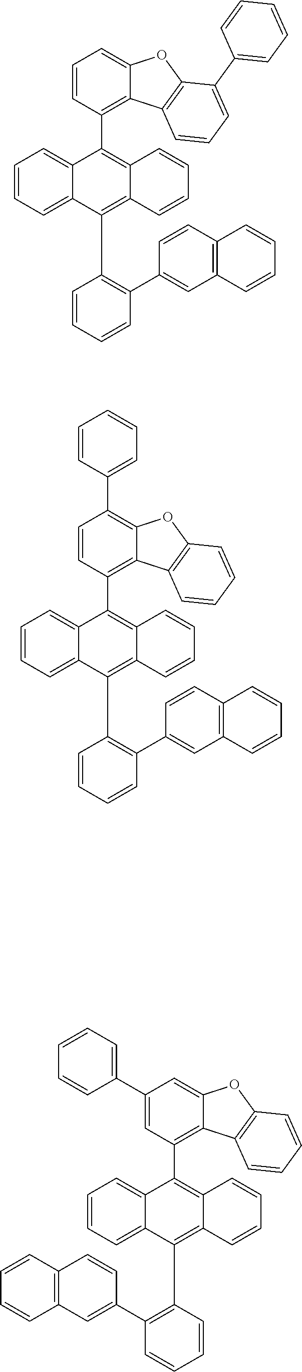 [SMILES: c1ccc(-c2cccc3c2oc2cccc(-c4c5ccccc5c(-c5ccccc5-c5ccc6ccccc6c5)c5ccccc45)c23)cc1 ; c1ccc(-c2ccc(-c3c4ccccc4c(-c4ccccc4-c4ccc5ccccc5c4)c4ccccc34)c3c2oc2ccccc23)cc1 ; c1ccc(-c2cc(-c3c4ccccc4c(-c4ccccc4-c4ccc5ccccc5c4)c4ccccc34)c3c(c2)oc2ccccc23)cc1]

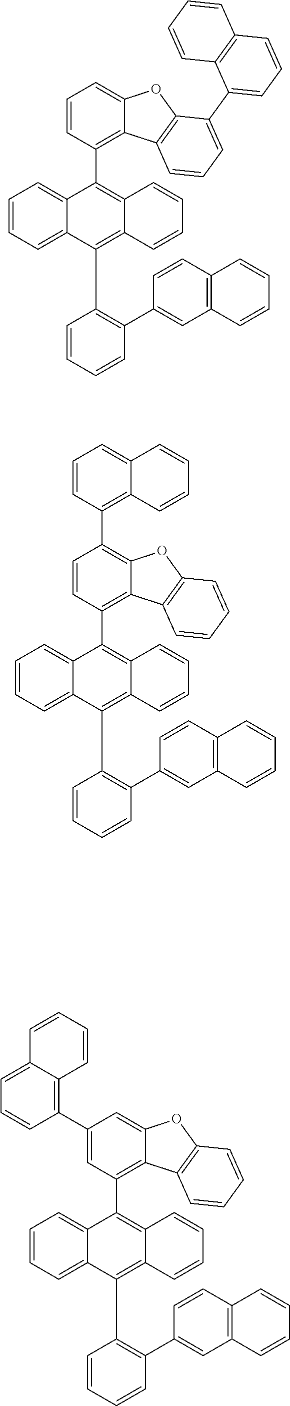 [SMILES: c1ccc(-c2c3ccccc3c(-c3cccc4oc5c(-c6cccc7ccccc67)cccc5c34)c3ccccc23)c(-c2ccc3ccccc3c2)c1 ; c1ccc(-c2c3ccccc3c(-c3ccc(-c4cccc5ccccc45)c4oc5ccccc5c34)c3ccccc23)c(-c2ccc3ccccc3c2)c1 ; c1ccc(-c2c3ccccc3c(-c3cc(-c4cccc5ccccc45)cc4oc5ccccc5c34)c3ccccc23)c(-c2ccc3ccccc3c2)c1]

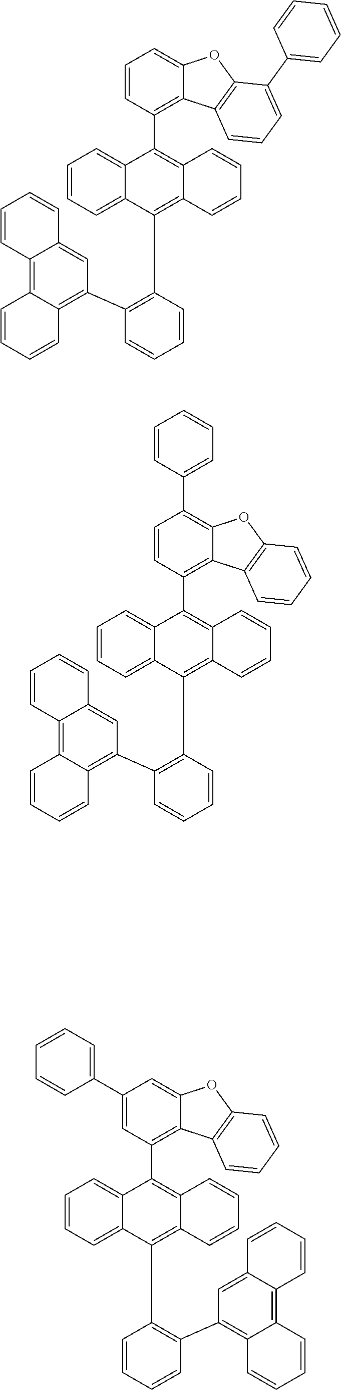 [SMILES: c1ccc(-c2cccc3c2oc2cccc(-c4c5ccccc5c(-c5ccccc5-c5cc6ccccc6c6ccccc56)c5ccccc45)c23)cc1 ; c1ccc(-c2ccc(-c3c4ccccc4c(-c4ccccc4-c4cc5ccccc5c5ccccc45)c4ccccc34)c3c2oc2ccccc23)cc1 ; c1ccc(-c2cc(-c3c4ccccc4c(-c4ccccc4-c4cc5ccccc5c5ccccc45)c4ccccc34)c3c(c2)oc2ccccc23)cc1]

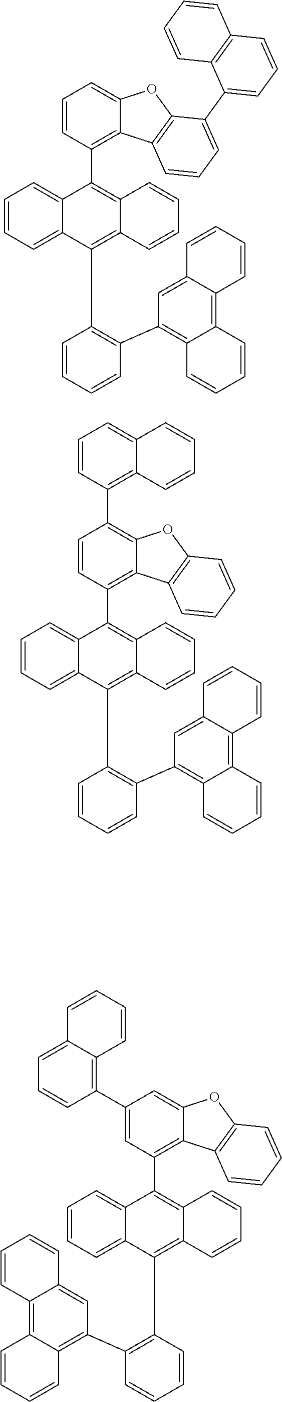

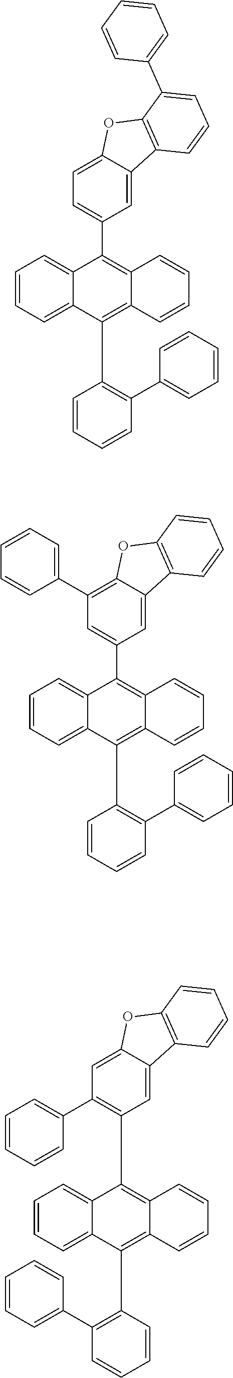 [SMILES: c1ccc(-c2ccccc2-c2c3ccccc3c(-c3ccc4oc5c(-c6ccccc6)cccc5c4c3)c3ccccc23)cc1 ; c1ccc(-c2ccccc2-c2c3ccccc3c(-c3cc(-c4ccccc4)c4oc5ccccc5c4c3)c3ccccc23)cc1 ; c1ccc(-c2ccccc2-c2c3ccccc3c(-c3cc4c(cc3-c3ccccc3)oc3ccccc34)c3ccccc23)cc1]

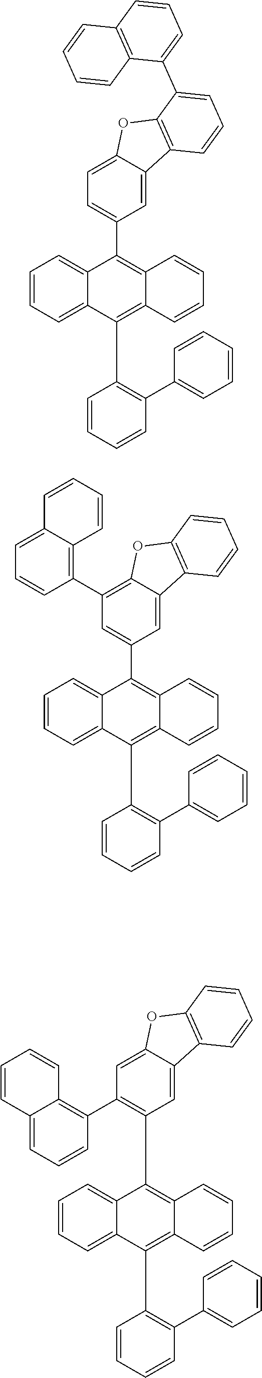 [SMILES: c1ccc(-c2ccccc2-c2c3ccccc3c(-c3ccc4oc5c(-c6cccc7ccccc67)cccc5c4c3)c3ccccc23)cc1 ; c1ccc(-c2ccccc2-c2c3ccccc3c(-c3cc(-c4cccc5ccccc45)c4oc5ccccc5c4c3)c3ccccc23)cc1 ; c1ccc(-c2ccccc2-c2c3ccccc3c(-c3cc4c(cc3-c3cccc5ccccc35)oc3ccccc34)c3ccccc23)cc1]

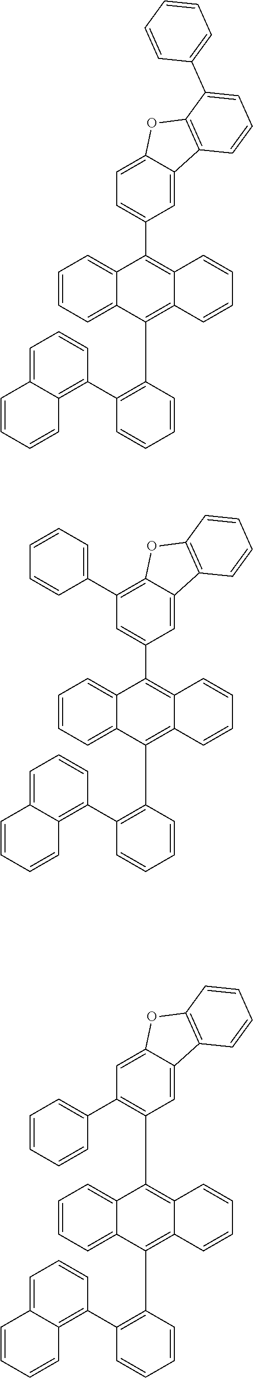 [SMILES: c1ccc(-c2cccc3c2oc2ccc(-c4c5ccccc5c(-c5ccccc5-c5cccc6ccccc56)c5ccccc45)cc23)cc1 ; c1ccc(-c2cc(-c3c4ccccc4c(-c4ccccc4-c4cccc5ccccc45)c4ccccc34)cc3c2oc2ccccc23)cc1 ; c1ccc(-c2cc3oc4ccccc4c3cc2-c2c3ccccc3c(-c3ccccc3-c3cccc4ccccc34)c3ccccc23)cc1]

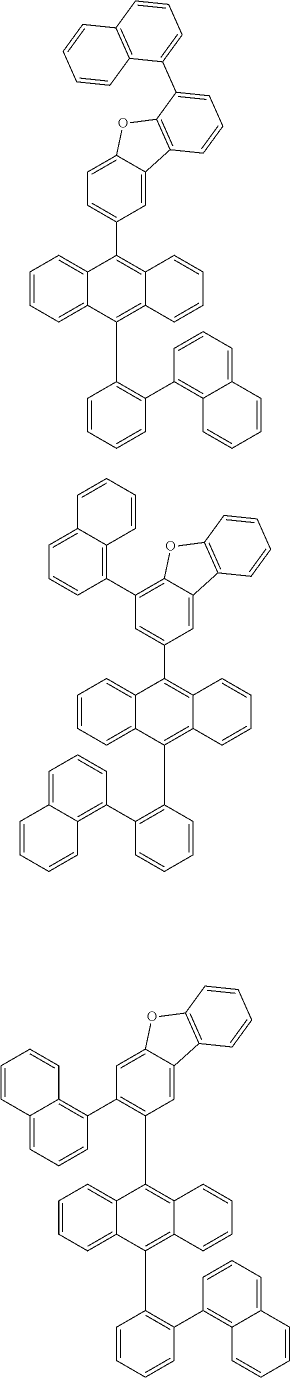

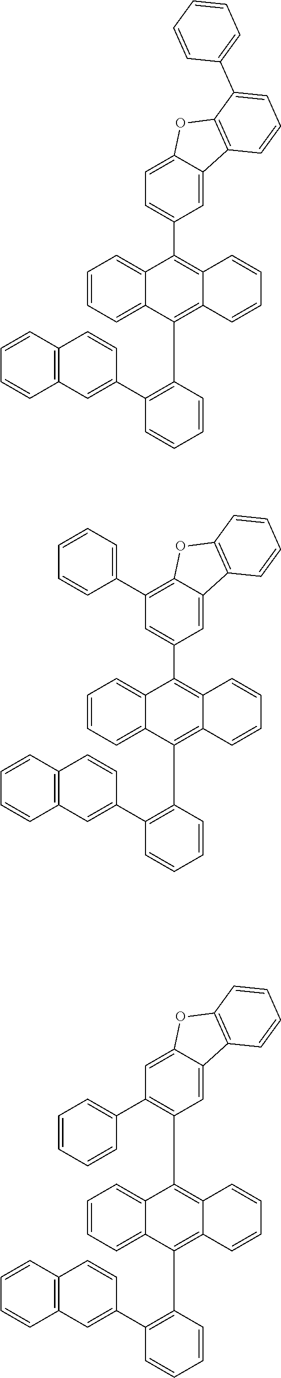 [SMILES: c1ccc(-c2cccc3c2oc2ccc(-c4c5ccccc5c(-c5ccccc5-c5ccc6ccccc6c5)c5ccccc45)cc23)cc1 ; c1ccc(-c2cc(-c3c4ccccc4c(-c4ccccc4-c4ccc5ccccc5c4)c4ccccc34)cc3c2oc2ccccc23)cc1 ; c1ccc(-c2cc3oc4ccccc4c3cc2-c2c3ccccc3c(-c3ccccc3-c3ccc4ccccc4c3)c3ccccc23)cc1]

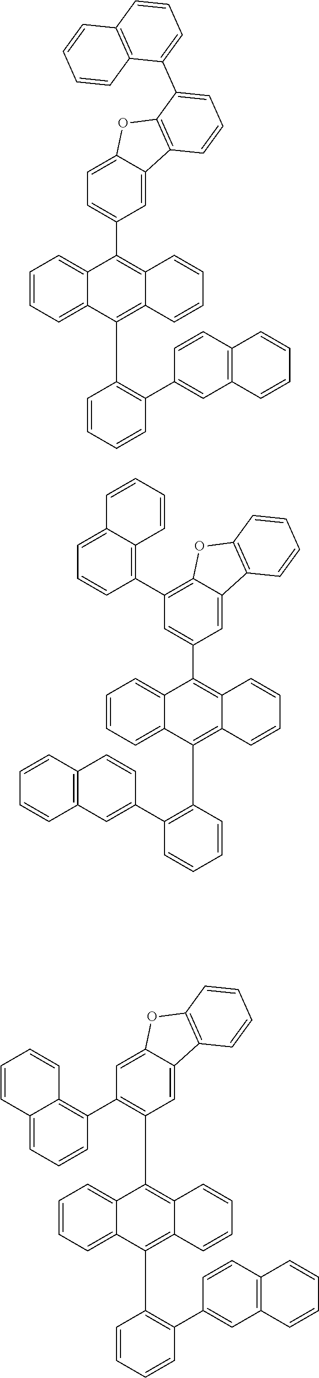

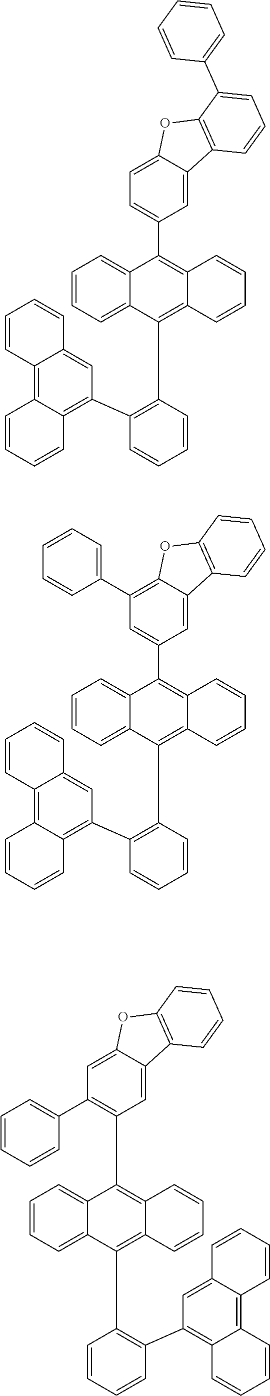 [SMILES: c1ccc(-c2cccc3c2oc2ccc(-c4c5ccccc5c(-c5ccccc5-c5cc6ccccc6c6ccccc56)c5ccccc45)cc23)cc1 ; c1ccc(-c2cc(-c3c4ccccc4c(-c4ccccc4-c4cc5ccccc5c5ccccc45)c4ccccc34)cc3c2oc2ccccc23)cc1 ; c1ccc(-c2cc3oc4ccccc4c3cc2-c2c3ccccc3c(-c3ccccc3-c3cc4ccccc4c4ccccc34)c3ccccc23)cc1]

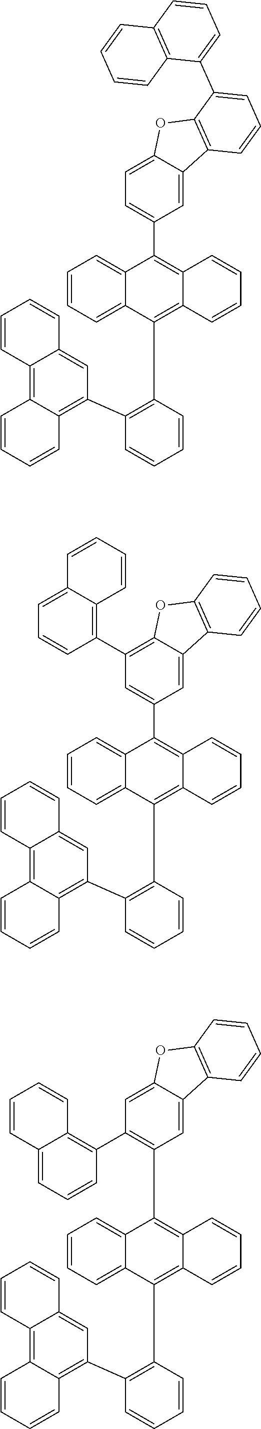 [SMILES: c1ccc(-c2cc3ccccc3c3ccccc23)c(-c2c3ccccc3c(-c3ccc4oc5c(-c6cccc7ccccc67)cccc5c4c3)c3ccccc23)c1 ; c1ccc(-c2cc3ccccc3c3ccccc23)c(-c2c3ccccc3c(-c3cc(-c4cccc5ccccc45)c4oc5ccccc5c4c3)c3ccccc23)c1 ; c1ccc(-c2cc3ccccc3c3ccccc23)c(-c2c3ccccc3c(-c3cc4c(cc3-c3cccc5ccccc35)oc3ccccc34)c3ccccc23)c1]

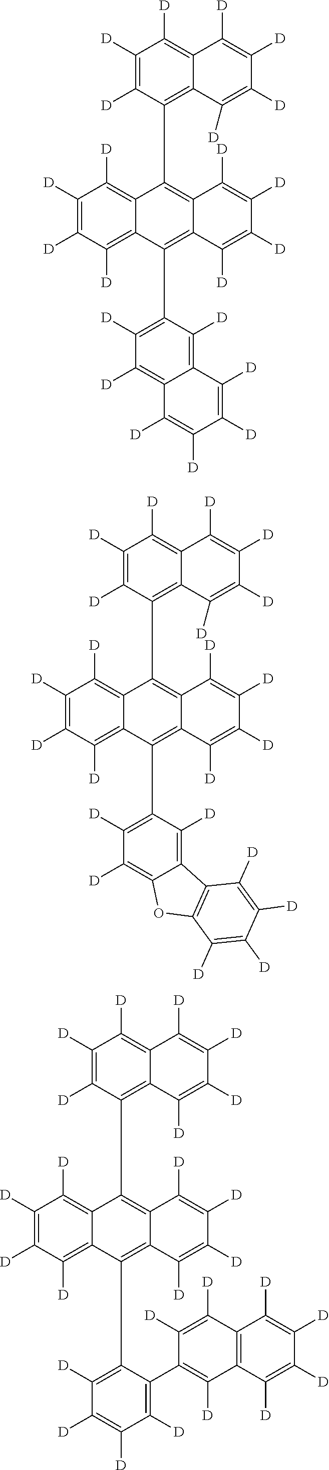

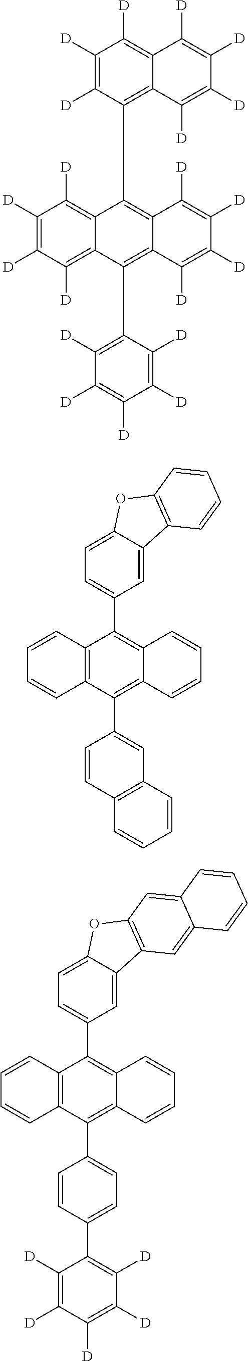

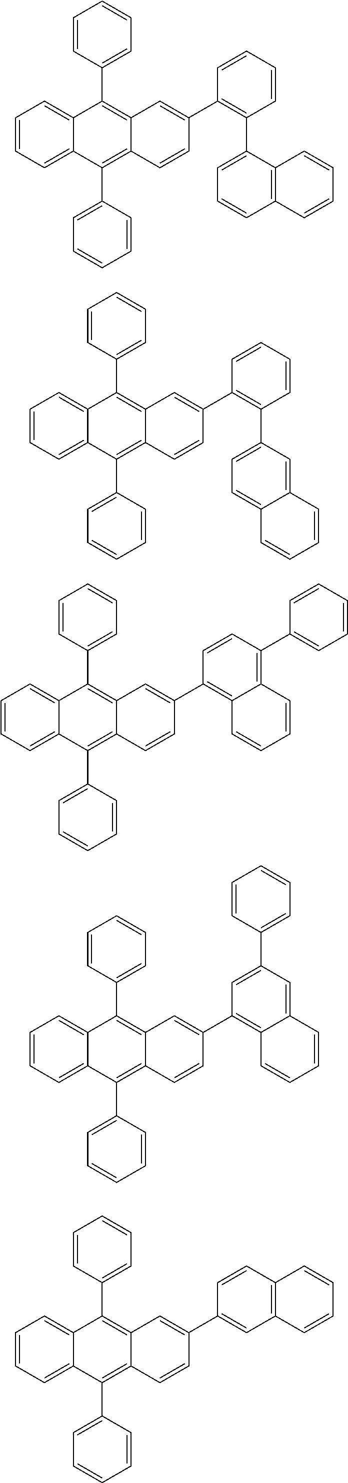

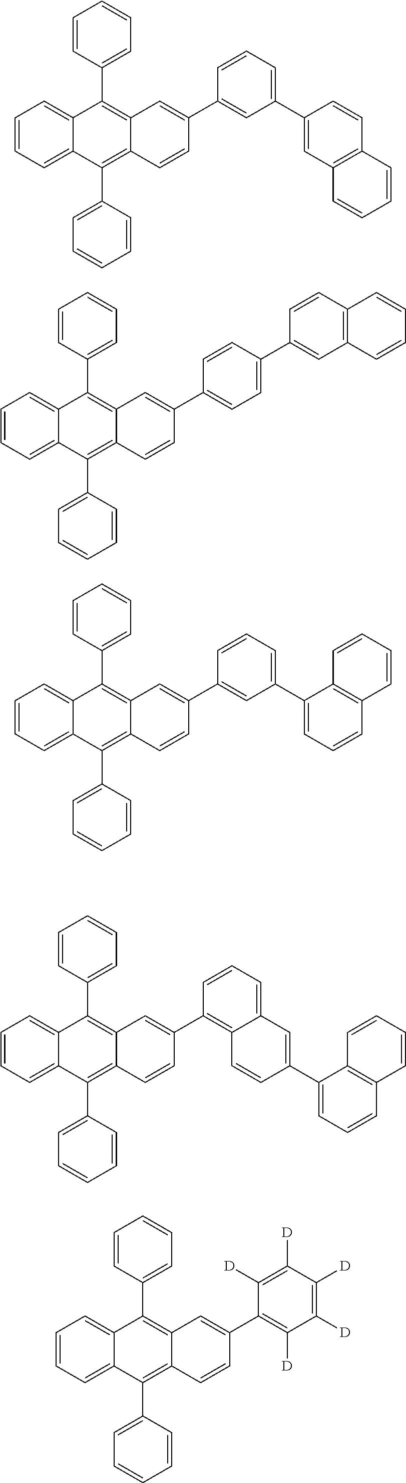

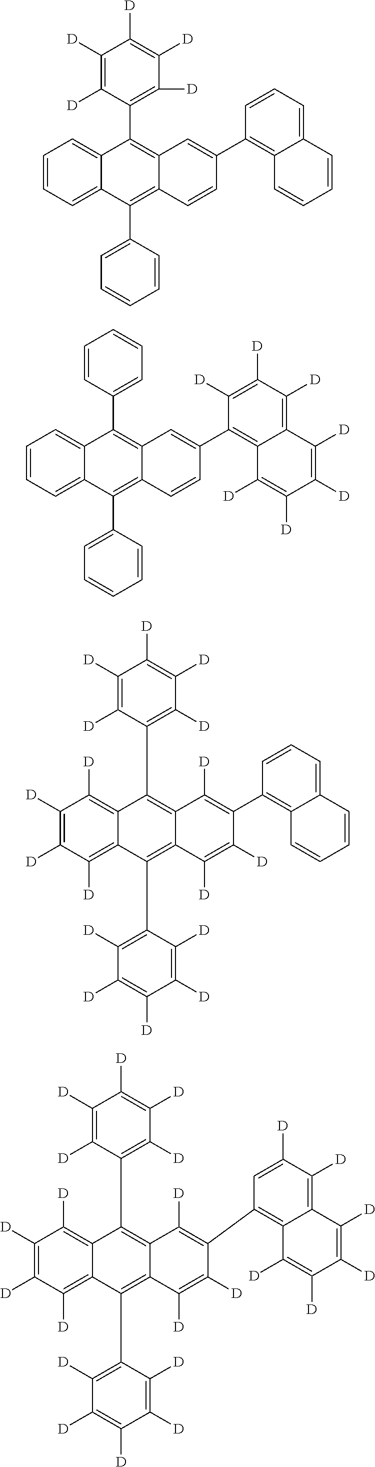

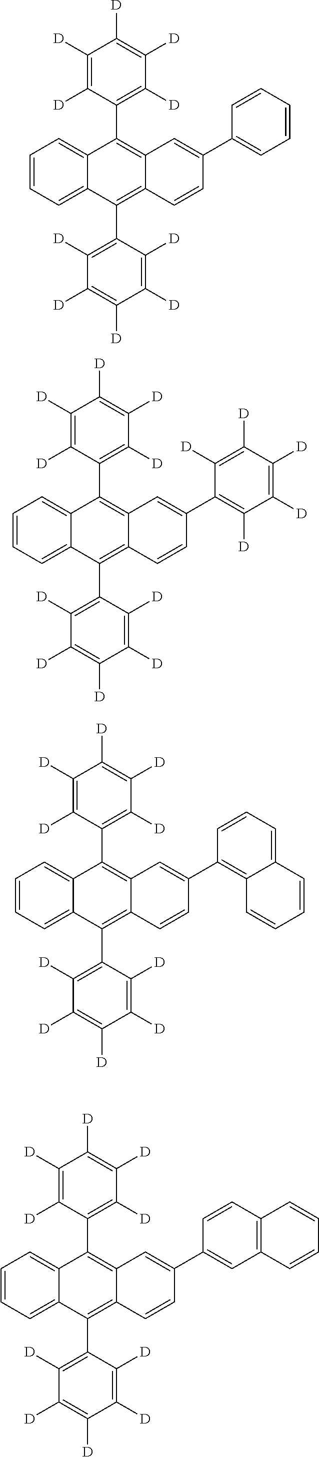

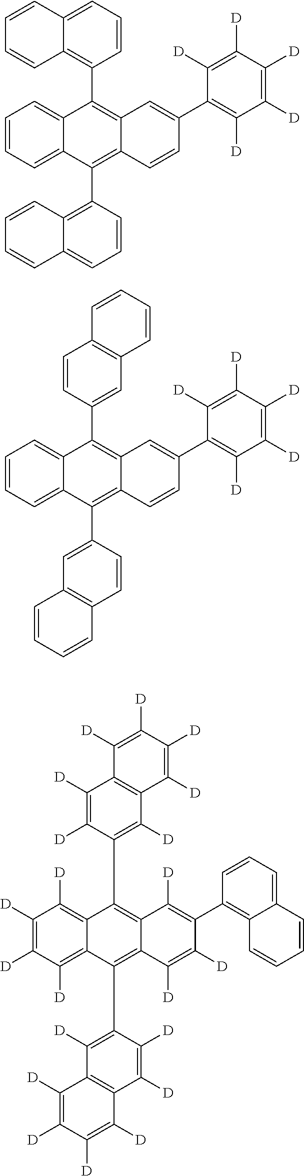

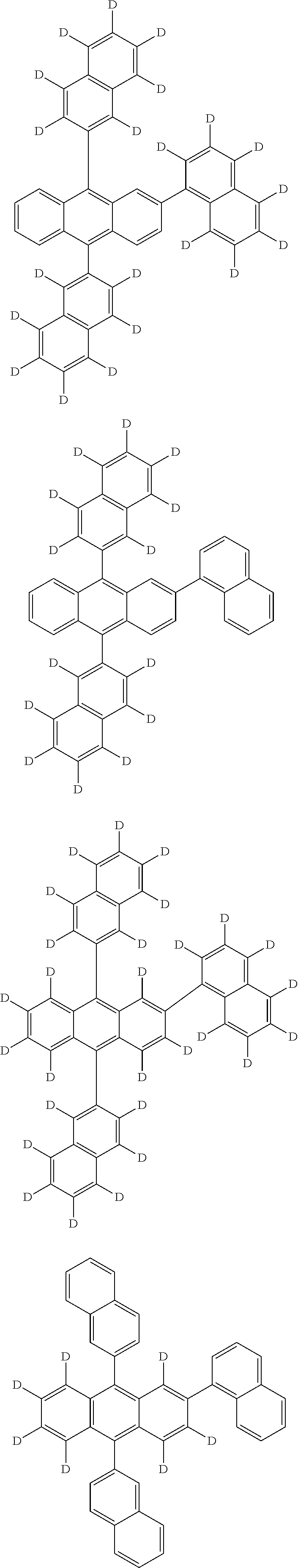

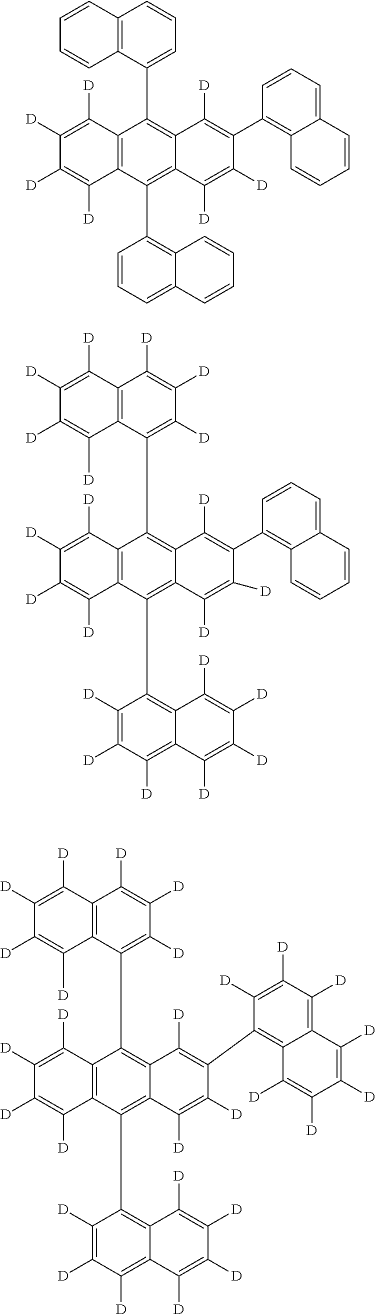

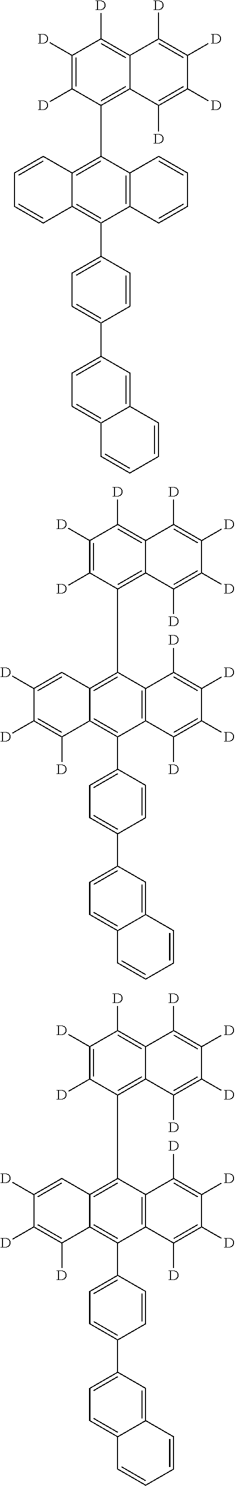

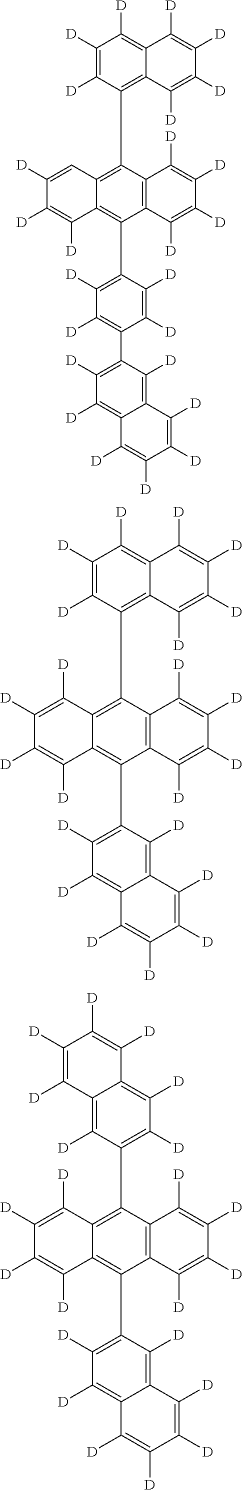

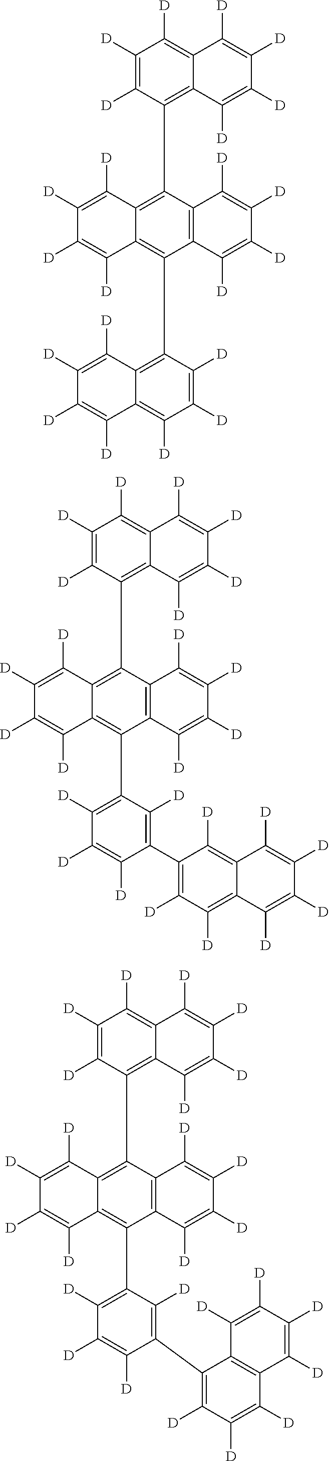

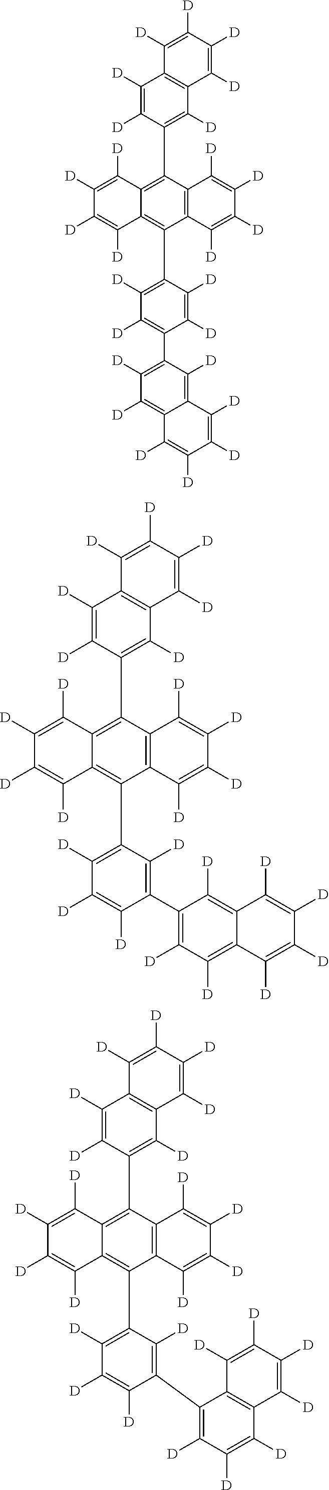

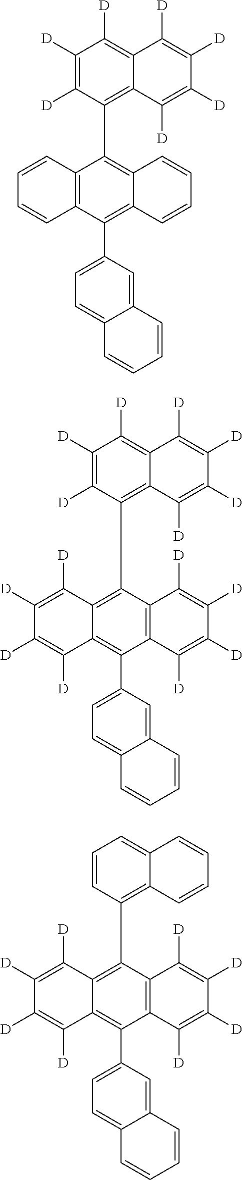

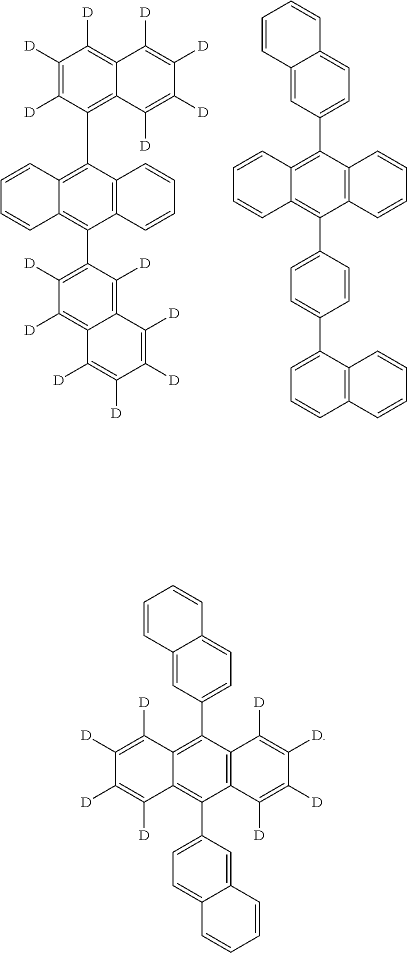

In an exemplary embodiment of the present specification, when the light emitting layer includes a host and a dopant, a content of the dopant can be selected within a range of 0.01 to 10 parts by weight based on 100 parts by weight of the light emitting layer, but is not limited thereto.

In an exemplary embodiment of the present specification, the light emitting layer includes a host and a dopant, and the host and the dopant are included at a weight ratio of 99:1 to 1:99, preferably 99:1 to 70:30, and more preferably 99:1 to 90:10.

In an exemplary embodiment of the present specification, a weight ratio of the host and the dopant is 99:1 to 90:10.

The organic light emitting device according to an exemplary embodiment of the present specification can include an additional light emitting layer in addition to a light emitting layer including the compound of Chemical Formula 1. In this case, the additional light emitting layer includes a phosphorescent dopant or a fluorescent dopant, and includes a phosphorescent host or a fluorescent dopant. The additional light emitting layer emits red, green or blue light.

The light emitting layer can further include a host material, and examples of the host include a fused aromatic ring derivative, a hetero ring-containing compound, and the like. Specifically, examples of the fused aromatic ring derivative include anthracene derivatives, pyrene derivatives, naphthalene derivatives, pentacene derivatives, phenanthrene compounds, fluoranthene compounds, and the like, and examples of the hetero ring-containing compound include carbazole derivatives, dibenzofuran derivatives, ladder-type furan compounds, pyrimidine derivatives, triazine derivatives, or the like, and the examples thereof can include a mixture of two or more thereof, but are not limited thereto.

According to an exemplary embodiment of the present specification, the light emitting layer includes one or more hosts.

According to an exemplary embodiment of the present specification, the light emitting layer includes two or more mixed hosts.

In an exemplary embodiment of the present specification, the organic light emitting device can be a normal type organic light emitting device in which an anode, an organic material layer having one or more layers, and a cathode are sequentially stacked on a substrate.

In an exemplary embodiment of the present specification, the organic light emitting device can be an inverted type organic light emitting device in which an anode, an organic material layer having one or more layers, and a cathode are sequentially stacked on a substrate.

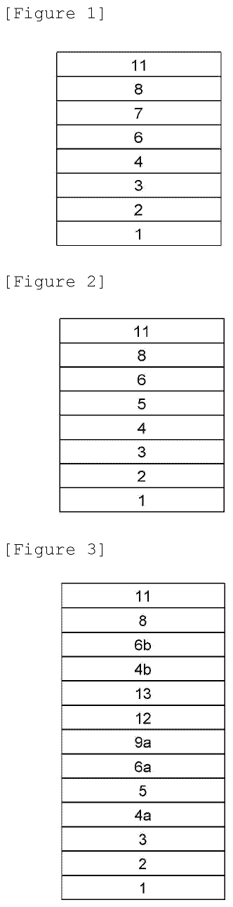

The structure of the organic light emitting device of the present specification can have a structure as illustrated in FIGS. 1, 2, 8, and 9, but is not limited thereto.

FIG. 1 illustrates a structure of an organic light emitting device in which a substrate 1, an anode 2, a hole injection layer 3, a hole transport layer 4, a light emitting layer 6, a hole blocking layer 7, an electron injection and transport layer 8, and a cathode 11 are sequentially stacked. In the structure as described above, the compound of Chemical Formula 1 can be included in the light emitting layer 6, and the compound of Chemical Formula 2 can be included in the hole injection layer 3 or the hole transport layer 4.

FIG. 2 illustrates a structure of an organic light emitting device in which a substrate 1, an anode 2, a hole injection layer 3, a hole transport layer 4, an electron blocking layer 5, a light emitting layer 6, an electron injection and transport layer 8, and a cathode 11 are sequentially stacked. In the structure as described above, the compound of Chemical Formula 1 can be included in the light emitting layer 6, and the compound of Chemical Formula 2 can be included in the hole injection layer 3, the hole transport layer 4, or the electron blocking layer 5.

FIG. 8 illustrates a structure of an organic light emitting device in which a substrate 1, an anode 2, a p-doped hole transport layer 4p, hole transport layers 4R, 4G, and 4B, light emitting layers 6RP, 6GP, and 6BF, a first electron transport layer 9a, a second electron transport layer 9b, an electron injection layer 10, a cathode 11, and a capping layer 14 are sequentially stacked. In the structure as described above, the compound of Chemical Formula 1 can be included in the light emitting layers 6RP, 6GP, and 6BF, and the compound of Chemical Formula 2 can be included in one or more layers of the p-doped hole transport layer 4p and the hole transport layers 4R, 4G, and 4B.

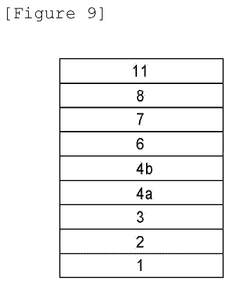

FIG. 9 illustrates a structure of an organic light emitting device in which a substrate 1, an anode 2, a hole injection layer 3, a first hole transport layer 4a, a first hole transport layer 4b, a light emitting layer 6, a hole blocking layer 7, an electron injection and transport layer 8, and a cathode 11 are sequentially stacked. In the structure as described above, the compound of Chemical Formula 1 can be included in the light emitting layer 6, and the compound of Chemical Formula 2 can be included in the hole injection layer 3, the first hole transport layer 4a, or the first hole transport layer 4b.

According to an exemplary embodiment of the present specification, the organic light emitting device can have a tandem structure in which two or more independent devices are connected in series. In an exemplary embodiment, the tandem structure can be in the form of each organic light emitting device joined by a charge generating layer. Since a device having a tandem structure can be driven with a current lower than that of a unit device based on the same brightness, there is an advantage in that the service life characteristic of the device is significantly improved.

According to an exemplary embodiment of the present specification, the organic material layer includes: a first stack including a light emitting layer having one or more layers; a second stack including a light emitting layer having one or more layers; and a charge generating layer having one or more layers provided between the first stack and the second stack.

According to another exemplary embodiment of the present specification, the organic material layer includes: a first stack including a light emitting layer having one or more layers; a second stack including a light emitting layer having one or more layers; a third stack including a light emitting layer having one or more layer, and includes a charge generating layer having one or more layers, between the first stack and the second stack; and between the second stack and the third stack, respectively.

In the present specification, the charge generating layer means a layer in which holes and electrons are generated when a voltage is applied. The charge generating layer can be an N-type charge generating layer or a P-type charge generating layer. In the present specification, an N-type charge generating layer means a charge generating layer located closer to an anode than a P-type charge generating layer, and a P-type charge generating layer means a charge generating layer located closer to a cathode than an N-type charge generating layer.

The N-type charge generating layer and the P-type charge generating layer can be provided to be brought into contact with each other, and in this case, form an NP junction. Holes and electrons are easily formed in the P-type charge generating layer and the N-type charge generating layer, respectively by the NP junction. Electrons are transported toward the anode through the LUMO level of the N-type charge generating layer, and holes are transported toward the cathode through the HOMO level of the P-type organic material layer.

The first stack, the second stack, and the third stack each include a light emitting layer having one or more layers, and can further include one or more layers of a hole injection layer, a hole transport layer, an electron blocking layer, an electron injection layer, an electron transport layer, a hole blocking layer, a layer which simultaneously transports and injects holes (a hole injection and transport layer), and a layer which simultaneously transports and injects electrons (an electron injection and transport layer).

An organic light emitting device including the first stack and the second stack is illustrated in FIG. 3.

FIG. 3 illustrates the structure of an organic light emitting device in which a substrate 1, an anode 2, a hole injection layer 3, a first hole transport layer 4a, an electron blocking layer 5, a first light emitting layer 6a, a first electron transport layer 9a, an N-type charge generating layer 12, a P-type charge generating layer 13, a second hole transport layer 4b, a second light emitting layer 6b, an electron injection and transport layer 8, and a cathode 11 are sequentially stacked. In the structure as described above, the compound of Chemical Formula 1 can be included in the first light emitting layer 6a or the second light emitting layer 6b, and the compound of Chemical Formula 2 can be included in the first hole transport layer 4a or the electron blocking layer 5.

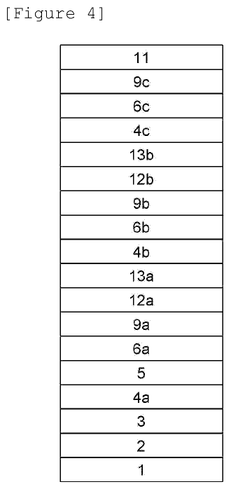

An organic light emitting device including the first stack to the third stack is illustrated in FIGS. 4 to 7.

FIG. 4 illustrates the structure of an organic light emitting device in which a substrate 1, an anode 2, a hole injection layer 3, a first hole transport layer 4a, an electron blocking layer 5, a first light emitting layer 6a, a first electron transport layer 9a, a first N-type charge generating layer 12a, a first P-type charge generating layer 13a, a second hole transport layer 4b, a second light emitting layer 6b, a second electron transport layer 9b, a second N-type charge generating layer 12b, a second P-type charge generating layer 13b, a third hole transport layer 4c, a third light emitting layer 6c, a third electron transport layer 9c, and a cathode 11 are sequentially stacked. In the structure as described above, the compound of Chemical Formula 1 can be included in the first light emitting layer 6a, the second light emitting layer 6b, and the third light emitting layer 6c, and the compound of Chemical Formula 2 can be included in one or more layers of the first hole transport layer 4a, the electron blocking layer 5, the second hole transport layer 4b, and the third hole transport layer 4c.

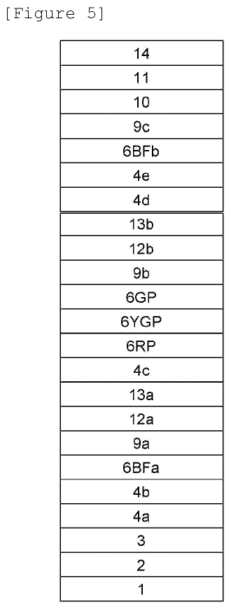

FIG. 5 illustrates the structure of an organic light emitting device in which a substrate 1, an anode 2, a hole injection layer 3, a first hole transport layer 4a, a second hole transport layer 4b, a first blue fluorescent light emitting layer 6BFa, a first electron transport layer 9a, a first N-type charge generating layer 12a, a first P-type charge generating layer 13a, a third hole transport layer 4c, a red phosphorescent light emitting layer 6RP, a yellow green phosphorescent light emitting layer 6YGP, a green phosphorescent light emitting layer 6GP, a second electron transport layer 9b, a second N-type charge generating layer 12b, a second P-type charge generating layer 13b, a fourth hole transport layer 4d, a fifth hole transport layer 4e, a second blue fluorescent light emitting layer 6BFb, a third electron transport layer 9c, an electron injection layer 10, a cathode 11, and a capping layer 14 are sequentially stacked. In the structure as described above, the compound of Chemical Formula 1 can be included in the first blue fluorescent light emitting layer 6BFa or the second blue fluorescent light emitting layer 6BFb, and the compound of Chemical Formula 2 can be include in the hole injection layer 3, the first hole transport layer 4a, the second hole transport layer 4b, the third hole transport layer 4c, the fourth hole transport layer 4d, or the fifth hole transport layer 4e.

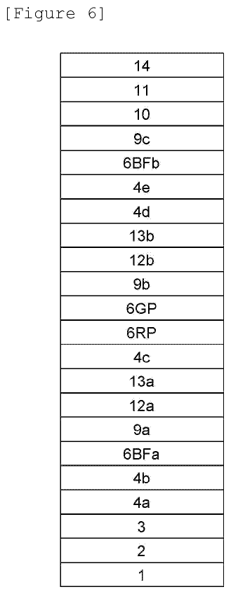

FIG. 6 illustrates the structure of an organic light emitting device in which a substrate 1, an anode 2, a hole injection layer 3, a first hole transport layer 4a, a second hole transport layer 4b, a first blue fluorescent light emitting layer 6BFa, a first electron transport layer 9a, a first N-type charge generating layer 12a, a first P-type charge generating layer 13a, a third hole transport layer 4c, a red phosphorescent light emitting layer 6RP, a green phosphorescent light emitting layer 6GP, a second electron transport layer 9b, a second N-type charge generating layer 12b, a second P-type charge generating layer 13b, a fourth hole transport layer 4d, a fifth hole transport layer 4e, a second blue fluorescent light emitting layer 6BFb, a third electron transport layer 9c, an electron injection layer 10, a cathode 11, and a capping layer 14 are sequentially stacked. In the structure as described above, the compound of Chemical Formula 1 can be included in the first blue fluorescent light emitting layer 6BFa or the second blue fluorescent light emitting layer 6BFb, and the compound of Chemical Formula 2 can be included in the hole injection layer 3, the first hole transport layer 4a, the second hole transport layer 4b, the third hole transport layer 4c, or the fourth hole transport layer 4d.

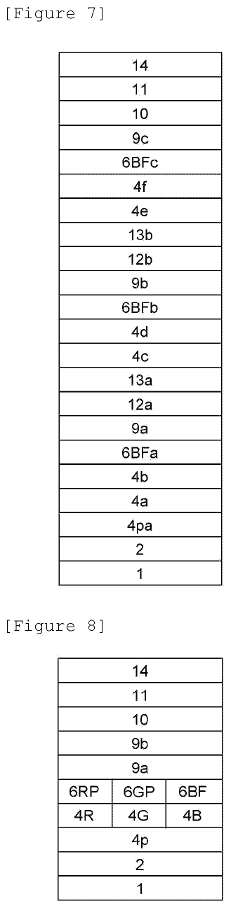

FIG. 7 illustrates the structure of an organic light emitting device in which a substrate 1, an anode 2, a p-doped first hole transport layer 4pa, a first hole transport layer 4a, a second hole transport layer 4b, a first blue fluorescent light emitting layer 6BFa, a first electron transport layer 9a, a first N-type charge generating layer 12a, a first P-type charge generating layer 13a, a third hole transport layer 4c, a fourth hole transport layer 4d, a second blue fluorescent light emitting layer 6BFb, a second electron transport layer 9b, a second N-type charge generating layer 12b, a second P-type charge generating layer 13b, a fifth hole transport layer 4e, a sixth hole transport layer 4f, a third blue fluorescent light emitting layer 6BFc, a third electron transport layer 9c, an electron injection layer 10, a cathode 11, and a capping layer 14 are sequentially stacked. In the structure as described above, the compound of Chemical Formula 1 can be included in one or more layers of the first blue fluorescent light emitting layer 6BFa, the second blue fluorescent light emitting layer 6BFb, and the third blue fluorescent light emitting layer 6BFc, and the compound of Chemical Formula 2 can be included in one or more layers of the p-doped first hole transport layer 4pa, the first hole transport layer 4a, the second hole transport layer 4b, the third hole transport layer 4c, the fourth hole transport layer 4d, the fifth hole transport layer 4e, and the sixth hole transport layer 4f.

The N-type charge generating layer can be 2,3,5,6-tetrafluoro-7,7,8,8-tetracyanoquinodimethane (F4TCNQ), fluorine-substituted 3,4,9,10-perylene-tetracarboxylic dianhydride (PTCDA), cyano-substituted PTCDA, naphthalene tetracarboxylic dianhydride (NTCDA), fluorine-substituted NTCDA, cyano-substituted NTCDA, hexaazatriphenylene derivatives, and the like, but is not limited thereto. In an exemplary embodiment, the N-type charge generating layer can include both benzoimidazophenanthridine-based derivatives and L1 metal.

The P-type charge generating layer can include both arylamine-based derivatives and a compound including a cyano group.

The organic light emitting device of the present specification can be manufactured by materials and methods known in the art, except that the organic material layer includes the compound.

When the organic light emitting device includes a plurality of organic material layers, the organic material layers can be formed of the same material or different materials.

For example, the organic light emitting device according to the present invention can be manufactured by depositing a metal or a metal oxide having conductivity, or an alloy thereof on a substrate to form an anode, forming an organic material layer having one or more layers selected from the group consisting of a hole injection layer, a hole transport layer, a layer which transports and injects holes simultaneously, a light emitting layer, an electron transport layer, an electron injection layer, and a layer which transports and injects electrons simultaneously, thereon, and then depositing a material, which can be used as a cathode, thereon, by using a physical vapor deposition (PVD) method such as sputtering or e-beam evaporation. In addition to the method described above, an organic light emitting device can be made by sequentially depositing a cathode material, an organic material layer, and an anode material on a substrate.

The organic material layer can have a multi-layered structure including a hole injection layer, a hole transport layer, a light emitting layer, and an electron transport layer, and the like, but is not limited thereto and can have a single-layered structure. Further, the organic material layer can be manufactured to include a fewer number of layers by a method such as a solvent process, for example, spin coating, dip coating, doctor blading, screen printing, inkjet printing, or a thermal transfer method, using various polymer materials, instead of a deposition method.

The anode is an electrode which injects holes, and as an anode material, materials having a high work function are usually preferred so as to facilitate the injection of holes into an organic material layer. Specific examples of the anode material which can be used in the present invention include: a metal, such as vanadium, chromium, copper, zinc, and gold, or an alloy thereof; a metal oxide, such as zinc oxide, indium oxide, indium tin oxide (ITO), and indium zinc oxide (IZO); a combination of a metal and an oxide, such as ZnO:Al or SnO₂:Sb; a conductive polymer, such as poly(3-methylthiophene), poly[3,4-(ethylene-1,2-dioxy)thiophene] (PEDOT), polypyrrole, and polyaniline; and the like, but are not limited thereto.

The cathode is an electrode which injects electrons, and as a cathode material, materials having a low work function are usually preferred so as to facilitate the injection of electrons into an organic material layer. Specific examples of the cathode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

The hole injection layer is a layer which serves to facilitate the injection of holes from an anode to a light emitting layer and can have a single-layered or multi-layered structure, and a hole injection material is preferably a material which can proficiently accept holes from an anode at a low voltage, and the highest occupied molecular orbital (HOMO) of the hole injection material is preferably a value between the work function of the anode material and the HOMO of the neighboring organic material layer. Specific examples of the hole injection material include metal porphyrin, oligothiophene, arylamine-based organic materials, hexanitrile hexaazatriphenylene-based organic materials, quinacridone-based organic materials, perylene-based organic materials, anthraquinone, polyaniline-based and polythiophene-based conductive polymers, and the like, but are not limited thereto. The hole injection layer can have a thickness of 1 to 150 nm. When the hole injection layer has a thickness of 1 nm or more, there is an advantage in that it is possible to prevent hole injection characteristics from deteriorating, and when the hole injection layer has a thickness of 150 nm or less, there is an advantage in that it is possible to prevent the driving voltage from being increased in order to improve the movement of holes due to the too thick hole injection layer. In an exemplary embodiment of the present specification, the hole injection layer has a multi-layered structure of two or more layers.

The hole transport layer can serve to facilitate the transport of holes. A hole transport material is suitably a material having high hole mobility which can accept holes from an anode or a hole injection layer and transfer the holes to a light emitting layer. Specific examples thereof include arylamine-based organic materials, conductive polymers, block copolymers having both conjugated portions and non-conjugated portions, and the like, but are not limited thereto.

A hole buffer layer can be additionally provided between a hole injection layer and a hole transport layer, and can include hole injection or transport materials known in the art.

An electron blocking layer can be provided between a hole transport layer and a light emitting layer. As the electron blocking layer, the above-described spiro compound or a material known in the art can be used.

The light emitting layer can emit red, green, or blue light, and can be composed of a phosphorescent material or a fluorescent material. The light emitting material is a material which can receive holes and electrons from a hole transport layer and an electron transport layer, respectively, and combine the holes and the electrons to emit light in a visible ray region, and is preferably a material having high quantum efficiency for fluorescence or phosphorescence.

In an exemplary embodiment of the present specification, the compound of Chemical Formula 1 can be used in a light emitting layer that emits blue light, and can be specifically used as a dopant together with a host in the light emitting layer that emits blue light.

In an organic light emitting device, when a plurality of light emitting layers are provided, specific examples of a compound that can be used for an additional light emitting layer include: 8-hydroxy-quinoline aluminum complexes (Alq₃); carbazole-based compounds; dimerized styryl compounds; BAlq; 10-hydroxybenzoquinoline-metal compounds; benzoxazole-based, benzthiazole-based and benzimidazole-based compounds; poly(p-phenylenevinylene) (PPV)-based polymers; spiro compounds; polyfluorene, rubrene, and the like, but are not limited thereto.

In this case, examples of a host material for the additional light emitting layer include fused aromatic ring derivatives, or hetero ring-containing compounds, and the like. Specifically, examples of the fused aromatic ring derivative include anthracene derivatives, pyrene derivatives, naphthalene derivatives, pentacene derivatives, phenanthrene compounds, fluoranthene compounds, and the like, and examples of the hetero ring-containing compound include carbazole derivatives, dibenzofuran derivatives, ladder-type furan compounds, pyrimidine derivatives, and the like, but the examples thereof are not limited thereto.

When the additional light emitting layer emits red light, it is possible to use a phosphorescent material such as bis(1-phenylisoquinoline)acetylacetonate iridium (PIQIr(acac)), bis(1-phenylquinoline)-acetylacetonate iridium (PQIr(acac)), tris(1-phenyl-quinoline)iridium (PQIr), or octaethylporphyrin platinum (PtOEP), or a fluorescent material such as tris(8-hydroxyquinolino)aluminum (Alq₃) as a light emitting dopant, but the light emitting dopant is not limited thereto. When the additional light emitting layer emits green light, it is possible to use a phosphorescent material such as fac tris(2-phenyl-pyridine)iridium (Ir(ppy)₃), or a fluorescent material such as tris(8-hydroxyquinolino)aluminum (Alq₃), as the light emitting dopant, but the light emitting dopant is not limited thereto. When the additional light emitting layer emits blue light, it is possible to use a phosphorescent material such as (4,6-F2ppy)₂Irpic, or a fluorescent material such as spiro-DPVBi, spiro-6P, distyryl benzene (DSB), distyryl arylene (DSA), a PFO-based polymer or a PPV-based polymer as the light emitting dopant, but the light emitting dopant is not limited thereto.

A hole blocking layer can be provided between the electron transport layer and the light emitting layer, and materials known in the art can be used.

The electron transport layer serves to facilitate the transport of electrons, and can have a single-layered or multi-layered structure. An electron transport material is suitably a material having high electron mobility which can proficiently accept electrons from a cathode and transfer the electrons to a light emitting layer. Specific examples thereof include: Al complexes of 8-hydroxyquinoline; complexes including Alq₃; organic radical compounds; hydroxyflavone-metal complexes; and the like, but are not limited thereto. The electron transport layer can have a thickness of 1 to 50 nm. When the electron transport layer has a thickness of 1 nm or more, there is an advantage in that it is possible to prevent electron transport characteristics from deteriorating, and when the electron transport layer has a thickness of 50 nm or less, there is an advantage in that it is possible to prevent the driving voltage from being increased in order to improve the movement of electrons due to the too thick electron transport layer. In an exemplary embodiment of the present specification, an electron transport layer has a multi-layered structure of two or more layers, and an electron transport layer adjacent to a cathode includes an n-type dopant.

The electron injection layer can serve to facilitate the injection of electrons. An electron injection material is preferably a compound which has a capability of transporting electrons, an effect of injecting electrons from a cathode, and an excellent effect of injecting electrons into a light emitting layer or a light emitting material, prevents excitons produced from a light emitting layer from moving to a hole injection layer, and is also excellent in the ability to form a thin film. Specific examples thereof include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, metal complex compounds, nitrogen-containing 5-membered ring derivatives, and the like, but are not limited thereto.

Examples of the metal complex compounds include 8-hydroxyquinolinato lithium, bis(8-hydroxyquinolinato) zinc, bis(8-hydroxyquinolinato) copper, bis(8-hydroxy-quinolinato) manganese, tris(8-hydroxyquinolinato) aluminum, tris(2-methyl-8-hydroxyquinolinato) aluminum, tris(8-hydroxyquinolinato) gallium, bis(10-hydroxy-benzo[h]quinolinato) beryllium, bis(10-hydroxybenzo[h]-quinolinato) zinc, bis (2-methyl-8-quinolinato) chlorogallium, bis(2-methyl-8-quinolinato) (o-cresolato) gallium, bis(2-methyl-8-quinolinato) (1-naphtholato) aluminum, bis(2-methyl-8-quinolinato) (2-naphtholato) gallium, and the like, but are not limited thereto.

The hole blocking layer is a layer which blocks holes from reaching a cathode, and can be generally formed under the same conditions as those of the hole injection layer. Specific examples thereof include oxadiazole derivatives or triazole derivatives, phenanthroline derivatives, BCP, aluminum complexes, and the like, but are not limited thereto.

The organic light emitting device according to the present invention can be a top emission type, a bottom emission type, or a dual emission type according to the material to be used.

### EXAMPLES

Hereinafter, the present specification will be described in detail with reference to Examples, Comparative Examples, and the like for specifically describing the present specification. However, the Examples and the Comparative Examples according to the present specification can be modified in various forms, and it is not interpreted that the scope of the present specification is limited to the Examples and the Comparative Examples described below in detail. The Examples and the Comparative Examples of the present specification are provided to more completely explain the present specification to a person with ordinary skill in the art.

### Synthesis Example of Chemical Formula 1

### Synthesis Example 1. Synthesis of Compound 1

### 1) Synthesis of Intermediate 1

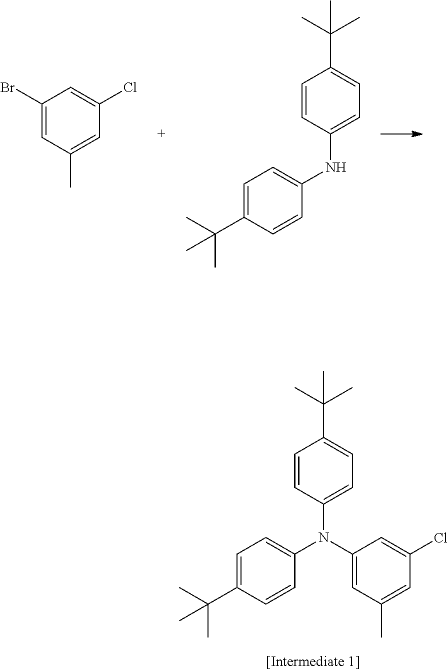

After 40 g of 1-bromo-3-chloro-5-methylbenzene, 54.8 g of bis(4-(tert-butyl)phenyl)amine, 56.1 g of sodium-tert-butoxide, and 1.0 g of bis(tri-tert-butylphosphine)-palladium(0) were put into 600 ml of toluene under a nitrogen atmosphere, the resulting mixture was stirred under reflux for 2 hours. After the completion of the reaction, the resulting product was extracted, and then recrystallized to obtain Intermediate 1 (65 g). (Yield 82%). MS[M+H]⁺=407

### 2) Synthesis of Intermediate 2

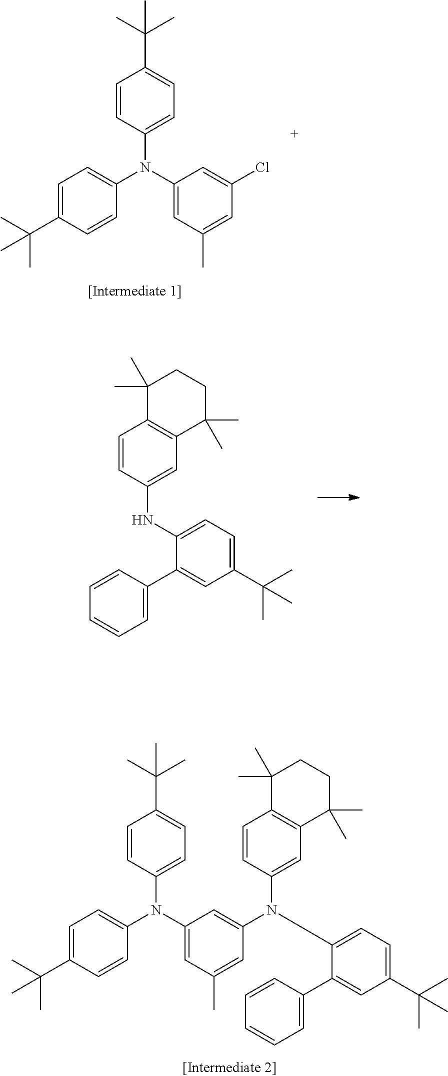 [SMILES: Cc1cc(Cl)cc(N(c2ccc(C(C)(C)C)cc2)c2ccc(C(C)(C)C)cc2)c1 ; CC(C)(C)c1ccc(Nc2ccc3c(c2)C(C)(C)CCC3(C)C)c(-c2ccccc2)c1 ; Cc1cc(N(c2ccc(C(C)(C)C)cc2)c2ccc(C(C)(C)C)cc2)cc(N(c2ccc3c(c2)C(C)(C)CCC3(C)C)c2ccc(C(C)(C)C)cc2-c2ccccc2)c1]

After Intermediate 1 (30 g), 30.5 g of N-(5-(tert-butyl)-[1,1′-biphenyl]-2-yl)-5,5,8,8-tetramethyl-5,6,7,8-tetrahydronaphthalen-2-amine, 14.2 g of sodium-tert-butoxide, and 0.4 g of bis(tri-tert-butylphosphine)palladium(0) were put into 450 ml of toluene under a nitrogen atmosphere, the resulting mixture was stirred under reflux for 2 hours. After the completion of the reaction, the resulting product was extracted, and then recrystallized to obtain Intermediate 2 (45 g). (Yield 78%). MS[M+H]⁺=782

### 3) Synthesis of Compound 1

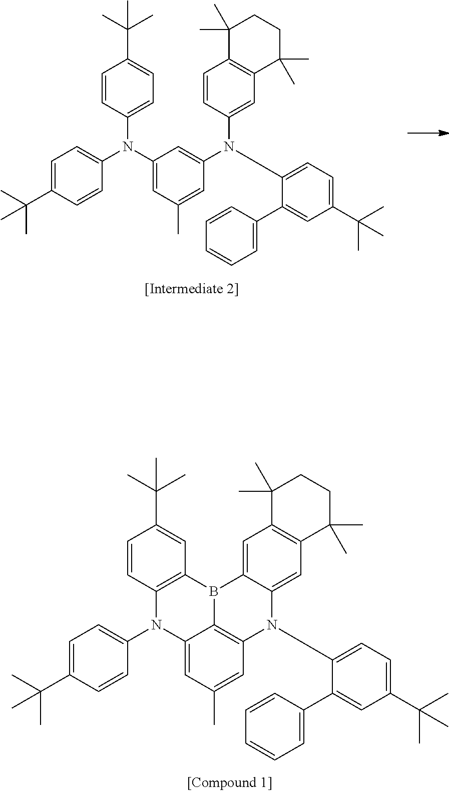

After Intermediate 2 (25 g) and 21.3 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Compound 1 (8 g) (yield 32%). MS[M+H]+=789

### Synthesis Example 2. Synthesis of Compound 2

### 1) Synthesis of Intermediate 3

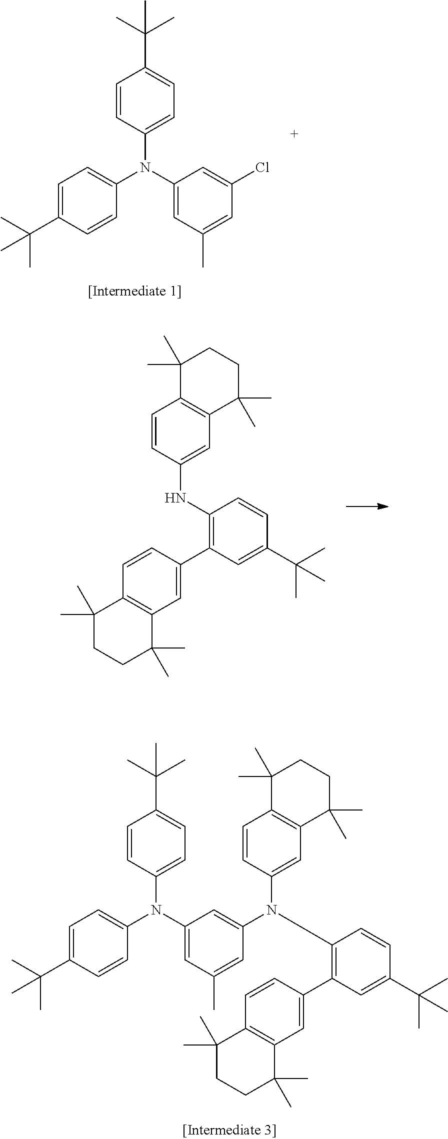 [SMILES: Cc1cc(Cl)cc(N(c2ccc(C(C)(C)C)cc2)c2ccc(C(C)(C)C)cc2)c1 ; CC(C)(C)c1ccc(Nc2ccc3c(c2)C(C)(C)CCC3(C)C)c(-c2ccc3c(c2)C(C)(C)CCC3(C)C)c1 ; Cc1cc(N(c2ccc(C(C)(C)C)cc2)c2ccc(C(C)(C)C)cc2)cc(N(c2ccc3c(c2)C(C)(C)CCC3(C)C)c2ccc(C(C)(C)C)cc2-c2ccc3c(c2)C(C)(C)CCC3(C)C)c1]

From Intermediate 1 (30 g) and 38.6 g of N-(4-(tert-butyl)-2-(5,5,8,8-tetramethyl-5,6,7,8-tetrahydronaphthalen-2-yl)phenyl)-5,5,8,8-tetramethyl-5,6,7,8-tetrahydronaphthalen-2-amine, Intermediate 3 (46 g) was obtained using the same material and equivalent weight as in the synthesis method of Intermediate 2 and using the same method as the synthesis method of Intermediate 2. (Yield 70%). MS[M+H]+=892

### 2) Synthesis of Compound 2

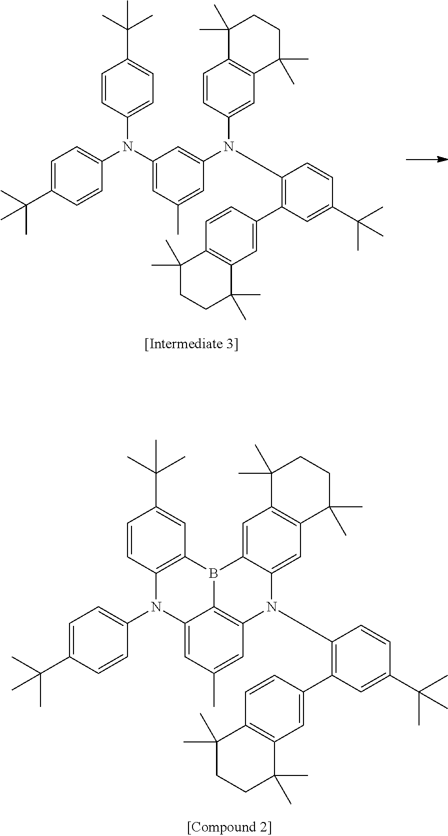

After Intermediate 3 (25 g) and 20.2 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Compound 2 (8.1 g) (yield 31%). MS[M+H]⁺=900

### Synthesis Example 3. Synthesis of Compound 3

### 1) Synthesis of Intermediate 4

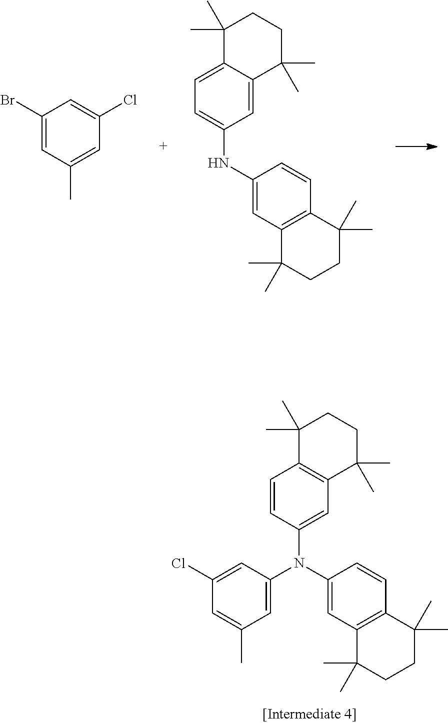

From 40 g of 1-bromo-3-chloro-5-methylbenzene and 75.8 g of bis(5,5,8,8-tetramethyl-5,6,7,8-tetrahydronaphthalen-2-yl)amine, Intermediate 4 (72 g) was obtained using the same material and equivalent weight as in the synthesis method of Intermediate 1 and using the same method as the synthesis method of Intermediate 1. (Yield 72%). MS[M+H]⁺=515

### 2) Synthesis of Intermediate 5

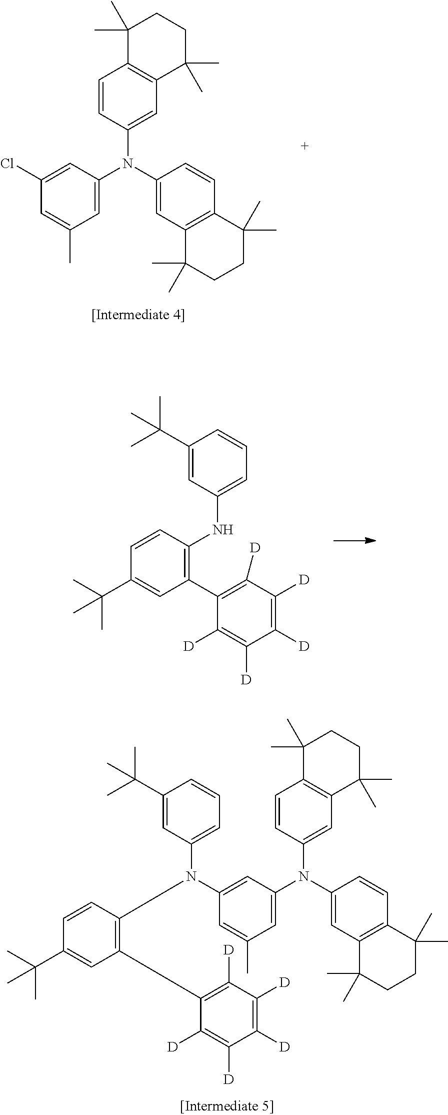 [SMILES: Cc1cc(Cl)cc(N(c2ccc3c(c2)C(C)(C)CCC3(C)C)c2ccc3c(c2)C(C)(C)CCC3(C)C)c1 ; [2H]c1c([2H])c([2H])c(-c2cc(C(C)(C)C)ccc2Nc2cccc(C(C)(C)C)c2)c([2H])c1[2H] ; [2H]c1c([2H])c([2H])c(-c2cc(C(C)(C)C)ccc2N(c2cc(C)cc(N(c3ccc4c(c3)C(C)(C)CCC4(C)C)c3ccc4c(c3)C(C)(C)CCC4(C)C)c2)c2cccc(C(C)(C)C)c2)c([2H])c1[2H]]

From Intermediate 4 (30 g) and 20.9 g of 5-(tert-butyl)-N-(3-(tert-butyl)phenyl)-[1,1′-biphenyl]-2-amine, Intermediate 5 (39 g) was obtained using the same material and equivalent weight as in the synthesis method of Intermediate 2 and using the same method as the synthesis method of Intermediate 2. (Yield 80%). MS[M+H]⁺=840

### 3) Synthesis of Compound 3

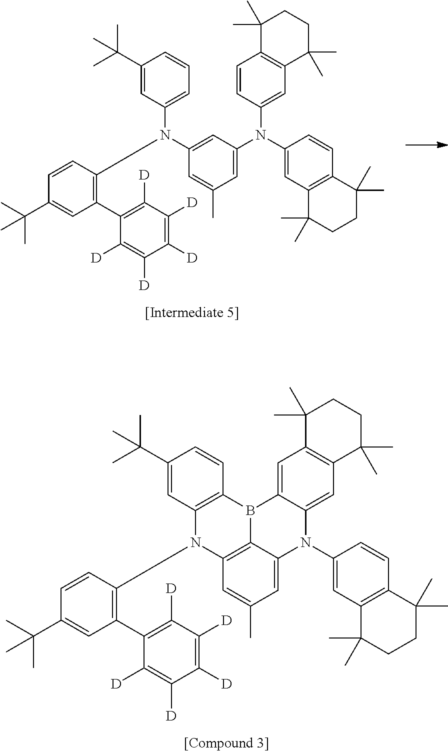

After Intermediate 5 (25 g) and 19.9 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Compound 3 (8.1 g) (yield 32%). MS[M+H]⁺=849

### Synthesis Example 4. Synthesis of Compound 4

### 1) Synthesis of Intermediate 5-1

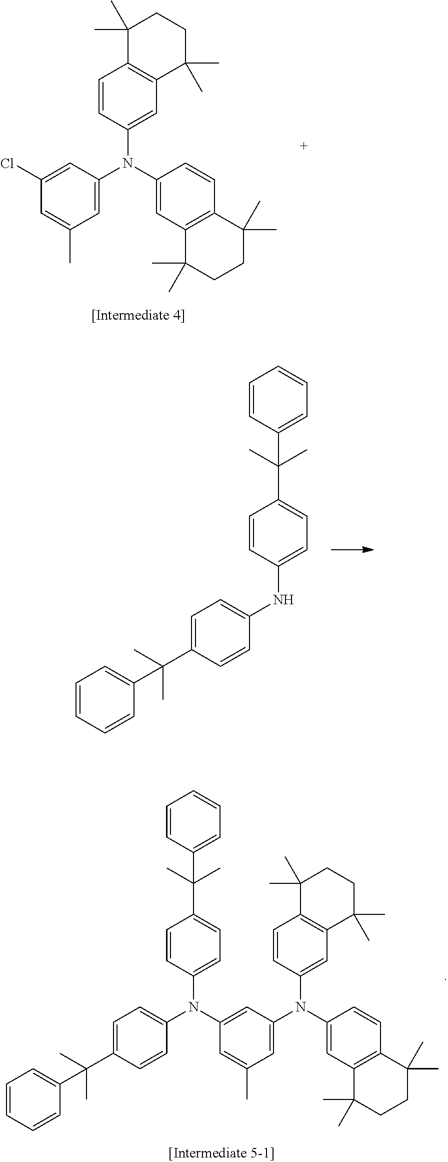 [SMILES: Cc1cc(Cl)cc(N(c2ccc3c(c2)C(C)(C)CCC3(C)C)c2ccc3c(c2)C(C)(C)CCC3(C)C)c1 ; CC(C)(c1ccccc1)c1ccc(Nc2ccc(C(C)(C)c3ccccc3)cc2)cc1 ; Cc1cc(N(c2ccc(C(C)(C)c3ccccc3)cc2)c2ccc(C(C)(C)c3ccccc3)cc2)cc(N(c2ccc3c(c2)C(C)(C)CCC3(C)C)c2ccc3c(c2)C(C)(C)CCC3(C)C)c1]

From Intermediate 4 (30 g) and 23.7 g of bis (4-(2-phenylpropan-2-yl)phenyl)amine, Intermediate 5-1 (36 g) was obtained using the same material and equivalent weight as in the synthesis method of Intermediate 2 and using the same method as the synthesis method of Intermediate 2. (Yield 70%). MS[M+H]+=884

### 2) Synthesis of Compound 4

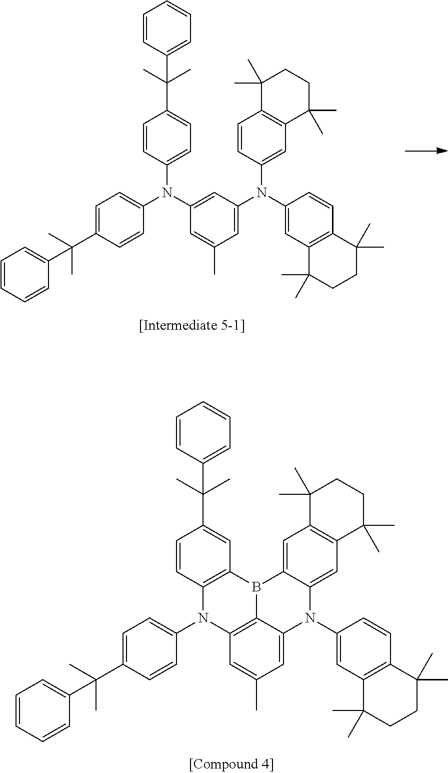 [SMILES: Cc1cc(N(c2ccc(C(C)(C)c3ccccc3)cc2)c2ccc(C(C)(C)c3ccccc3)cc2)cc(N(c2ccc3c(c2)C(C)(C)CCC3(C)C)c2ccc3c(c2)C(C)(C)CCC3(C)C)c1 ; Cc1cc2c3c(c1)N(c1ccc4c(c1)C(C)(C)CCC4(C)C)c1cc4c(cc1B3c1cc(C(C)(C)c3ccccc3)ccc1N2c1ccc(C(C)(C)c2ccccc2)cc1)C(C)(C)CCC4(C)C]

After Intermediate 5-1 (25 g) and 18.8 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Compound 4 (7.6 g) (yield 30%). MS[M+H]⁺=892

### Synthesis Example 5. Synthesis of Compound 5

### 1) Synthesis of Intermediate 6

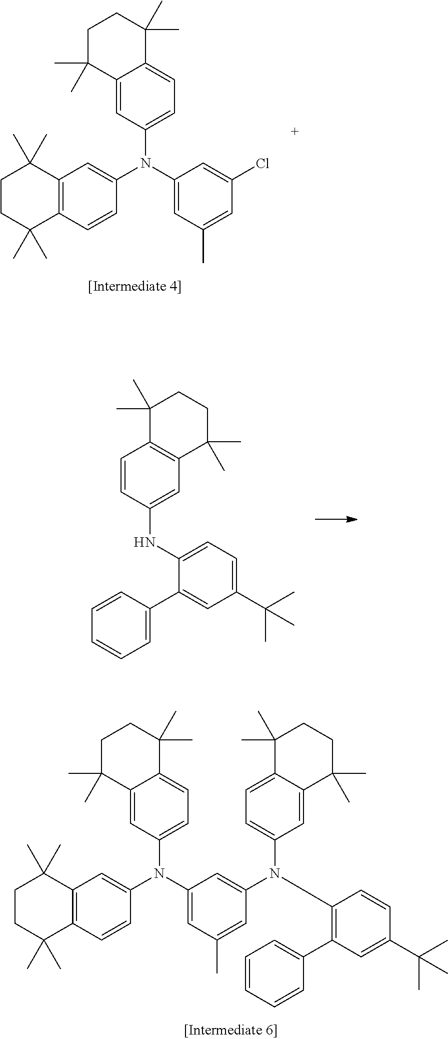 [SMILES: Cc1cc(Cl)cc(N(c2ccc3c(c2)C(C)(C)CCC3(C)C)c2ccc3c(c2)C(C)(C)CCC3(C)C)c1 ; CC(C)(C)c1ccc(Nc2ccc3c(c2)C(C)(C)CCC3(C)C)c(-c2ccccc2)c1 ; Cc1cc(N(c2ccc3c(c2)C(C)(C)CCC3(C)C)c2ccc3c(c2)C(C)(C)CCC3(C)C)cc(N(c2ccc3c(c2)C(C)(C)CCC3(C)C)c2ccc(C(C)(C)C)cc2-c2ccccc2)c1]

From Intermediate 4 (30 g) and 24.7 g of N-(5-(tert-butyl)-[1,1′-biphenyl]-2-yl)-5,5,8,8-tetramethyl-5,6,7,8-tetrahydronaphthalen-2-amine, Intermediate 6 (39 g) was obtained using the same material and equivalent weight as in the synthesis method of Intermediate 2 and using the same method as the synthesis method of Intermediate 2. (Yield 75%). MS[M+H]+=890

### 2) Synthesis of Compound 5

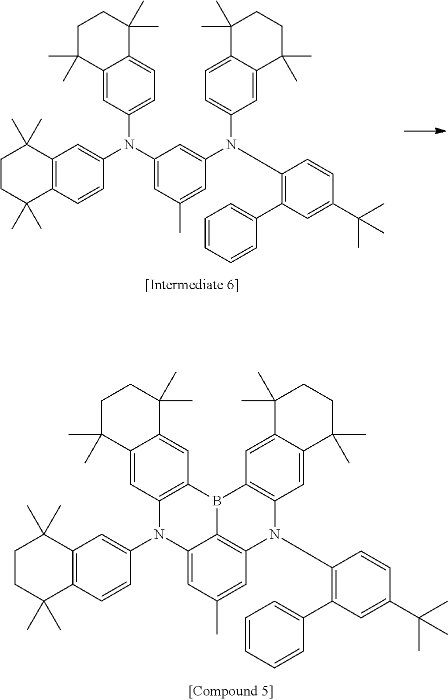 [SMILES: Cc1cc(N(c2ccc3c(c2)C(C)(C)CCC3(C)C)c2ccc3c(c2)C(C)(C)CCC3(C)C)cc(N(c2ccc3c(c2)C(C)(C)CCC3(C)C)c2ccc(C(C)(C)C)cc2-c2ccccc2)c1 ; Cc1cc2c3c(c1)N(c1ccc(C(C)(C)C)cc1-c1ccccc1)c1cc4c(cc1B3c1cc3c(cc1N2c1ccc2c(c1)C(C)(C)CCC2(C)C)C(C)(C)CCC3(C)C)C(C)(C)CCC4(C)C]

After Intermediate 6 (25 g) and 18.7 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Compound 5 (7.2 g) (yield 29%). MS [M+H]+=898

### Synthesis Example 6. Synthesis of Compound 6

### 1) Synthesis of Intermediate 7

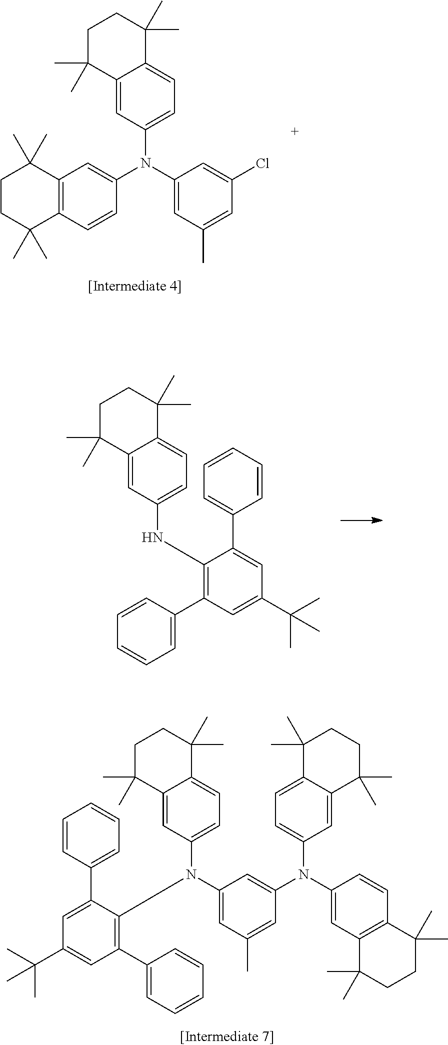 [SMILES: Cc1cc(Cl)cc(N(c2ccc3c(c2)C(C)(C)CCC3(C)C)c2ccc3c(c2)C(C)(C)CCC3(C)C)c1 ; CC(C)(C)c1cc(-c2ccccc2)c(Nc2ccc3c(c2)C(C)(C)CCC3(C)C)c(-c2ccccc2)c1 ; Cc1cc(N(c2ccc3c(c2)C(C)(C)CCC3(C)C)c2ccc3c(c2)C(C)(C)CCC3(C)C)cc(N(c2ccc3c(c2)C(C)(C)CCC3(C)C)c2c(-c3ccccc3)cc(C(C)(C)C)cc2-c2ccccc2)c1]

From Intermediate 4 (30 g) and 28.5 g of N-(5′-(tert-butyl)-[1,1′: 3′,1″-terphenyl]-2′-yl)-5,5,8,8-tetramethyl-5,6,7,8-tetrahydronaphthalen-2-amine, Intermediate 7 (42 g) was obtained using the same material and equivalent weight as in the synthesis method of Intermediate 2 and using the same method as the synthesis method of Intermediate 2. (Yield 75%). MS[M+H]+=966

### 2) Synthesis of Compound 6

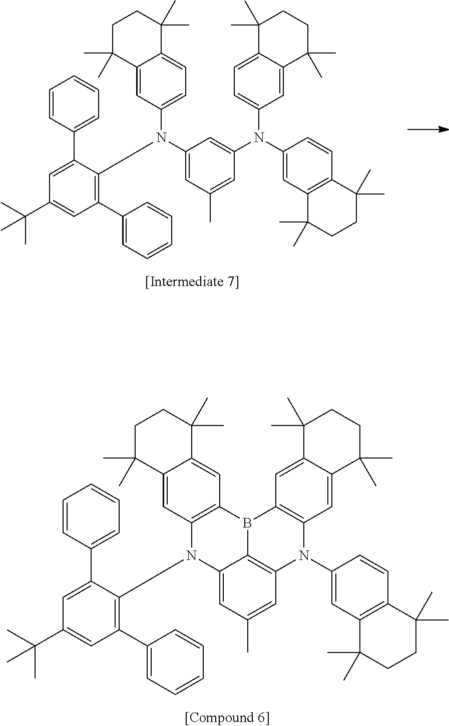

After Intermediate 7 (25 g) and 17.3 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Compound 6 (7.6 g) (yield 30%). MS [M+H]+=974

### Synthesis Example 7. Synthesis of Compound 7

### 1) Synthesis of Intermediate 8

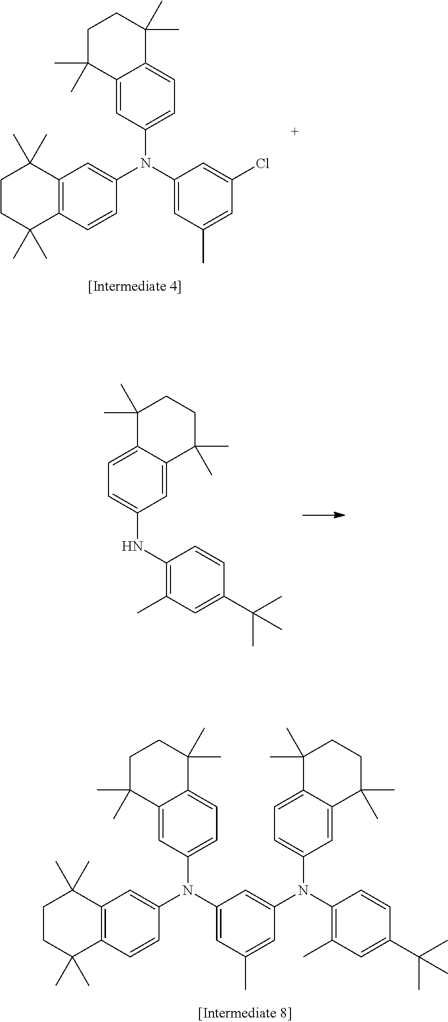 [SMILES: Cc1cc(Cl)cc(N(c2ccc3c(c2)C(C)(C)CCC3(C)C)c2ccc3c(c2)C(C)(C)CCC3(C)C)c1 ; Cc1cc(C(C)(C)C)ccc1Nc1ccc2c(c1)C(C)(C)CCC2(C)C ; Cc1cc(N(c2ccc3c(c2)C(C)(C)CCC3(C)C)c2ccc3c(c2)C(C)(C)CCC3(C)C)cc(N(c2ccc3c(c2)C(C)(C)CCC3(C)C)c2ccc(C(C)(C)C)cc2C)c1]

From Intermediate 4 (30 g) and 20.4 g of N-(4-(tert-butyl)-2-methylphenyl)-5,5,8,8-tetramethyl-5,6,7,8-tetrahydronaphthalen-2-amine, Intermediate 8 (33 g) was obtained using the same material and equivalent weight as in the synthesis method of Intermediate 2 and using the same method as the synthesis method of Intermediate 2. (Yield 68%). MS[M+H]+=828

### 2) Synthesis of Compound 7

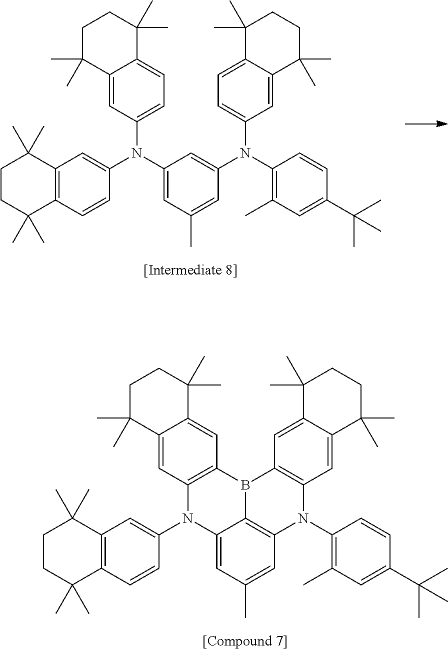 [SMILES: Cc1cc(N(c2ccc3c(c2)C(C)(C)CCC3(C)C)c2ccc3c(c2)C(C)(C)CCC3(C)C)cc(N(c2ccc3c(c2)C(C)(C)CCC3(C)C)c2ccc(C(C)(C)C)cc2C)c1 ; Cc1cc2c3c(c1)N(c1ccc(C(C)(C)C)cc1C)c1cc4c(cc1B3c1cc3c(cc1N2c1ccc2c(c1)C(C)(C)CCC2(C)C)C(C)(C)CCC3(C)C)C(C)(C)CCC4(C)C]

After Intermediate 8 (25 g) and 20.1 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Compound 7 (7.7 g) (yield 31%). MS[M+H]+=836

### Synthesis Example 8. Synthesis of Compound 8

### 1) Synthesis of Intermediate 9

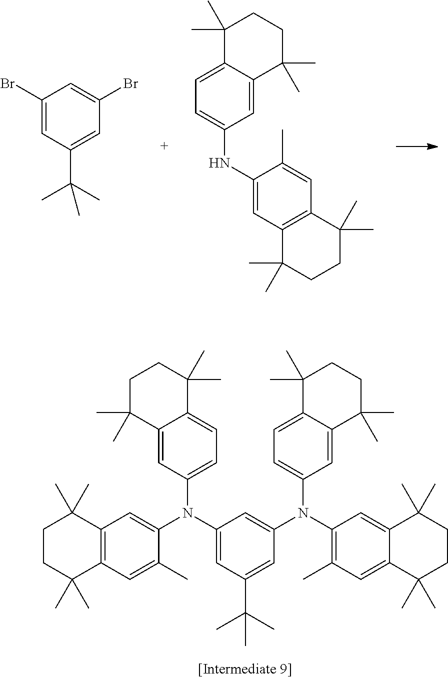

After 20 g of 1,3-dibromo-5-tert-butylbenzene, 55.3 g of 3,5,5,8,8-pentamethyl-N-(5,5,8,8-tetramethyl-5,6,7,8-tetrahydronaphthalen-2-yl)-5,6,7,8-tetrahydronaphthalen-2-amine, 16.5 g of sodium-tert-butoxide, and 1.0 g of bis(tri-tert-butylphosphine)-palladium(0) were put into 400 ml of toluene, the resulting mixture was stirred under reflux for 4 hours. After the completion of the reaction, the resulting product was extracted, and then recrystallized to obtain Intermediate 9 (41.7 g). (Yield 68%). MS[M+H]+=938

### 2) Synthesis of Compound 8

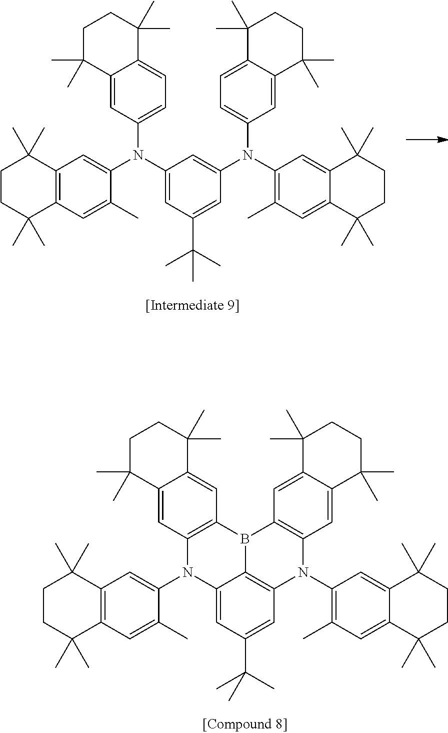 [SMILES: Cc1cc2c(cc1N(c1cc(N(c3ccc4c(c3)C(C)(C)CCC4(C)C)c3cc4c(cc3C)C(C)(C)CCC4(C)C)cc(C(C)(C)C)c1)c1ccc3c(c1)C(C)(C)CCC3(C)C)C(C)(C)CCC2(C)C ; Cc1cc2c(cc1N1c3cc4c(cc3B3c5cc6c(cc5N(c5cc7c(cc5C)C(C)(C)CCC7(C)C)c5cc(C(C)(C)C)cc1c53)C(C)(C)CCC6(C)C)C(C)(C)CCC4(C)C)C(C)(C)CCC2(C)C]

After Intermediate 9 (25 g) and 20.1 g of boron triiodide were put into 250 ml of 1,2-dichlorobenzene under a nitrogen atmosphere, the resulting mixture was stirred at 150° C. for 4 hours. After the completion of the reaction, the resulting product was extracted, and then recrystallized to obtain Compound 8 (7.8 g) (yield 31%). MS[M+H]+=946

### Synthesis Example 9. Synthesis of Compound 9

### 1) Synthesis of Intermediate 10

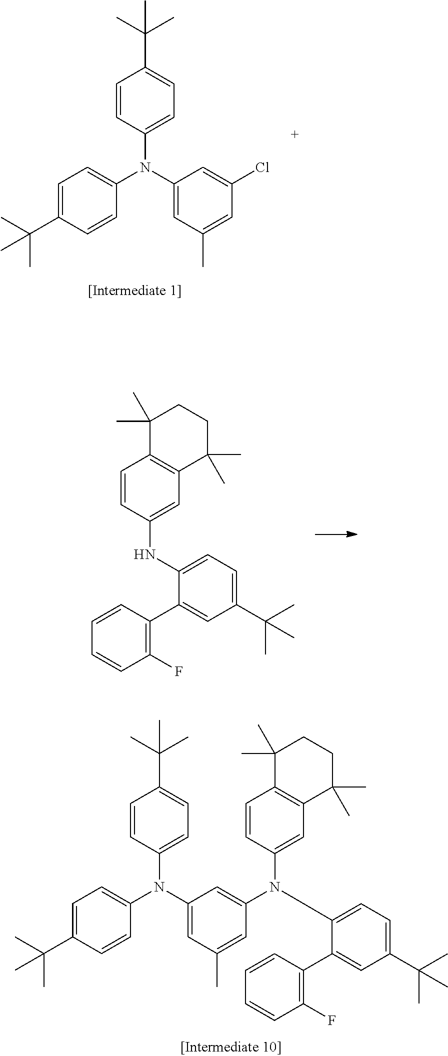 [SMILES: Cc1cc(Cl)cc(N(c2ccc(C(C)(C)C)cc2)c2ccc(C(C)(C)C)cc2)c1 ; CC(C)(C)c1ccc(Nc2ccc3c(c2)C(C)(C)CCC3(C)C)c(-c2ccccc2F)c1 ; Cc1cc(N(c2ccc(C(C)(C)C)cc2)c2ccc(C(C)(C)C)cc2)cc(N(c2ccc3c(c2)C(C)(C)CCC3(C)C)c2ccc(C(C)(C)C)cc2-c2ccccc2F)c1]

From Intermediate 1 (15 g) and 15.8 g of N-(5-(tert-butyl)-2′-fluoro-[1,1′-biphenyl]-2-yl)-5,5,8,8-tetramethyl-5,6,7,8-tetrahydronaphthalen-2-amine, Intermediate 10 (20.3 g) was obtained using the same material and equivalent weight as in the synthesis method of Intermediate 2 and using the same method as the synthesis method of Intermediate 2. (Yield 69%). MS[M+H]+=780

### 2) Synthesis of Compound 9

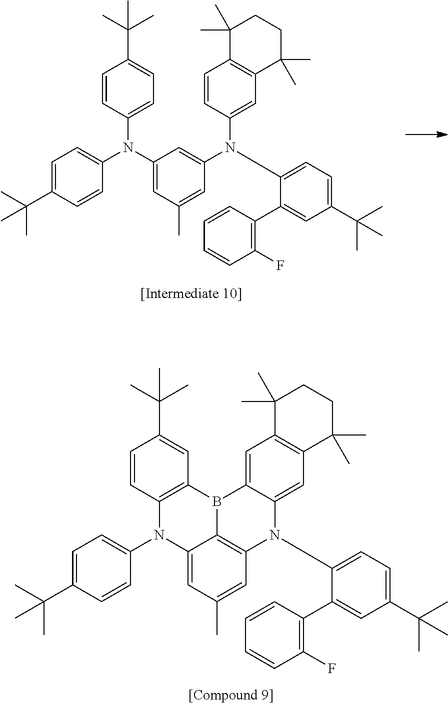

After Intermediate 10 (15 g) and 11 g of boron triiodide were put into 250 ml of 1,2-dichlorobenzene under a nitrogen atmosphere, the resulting mixture was stirred at 150° C. for 4 hours. After the completion of the reaction, the resulting product was extracted, and then recrystallized to obtain Compound 9 (5 g) (yield 33%). MS[M+H]+=808

### Synthesis Example 10. Synthesis of Compound 10

### 1) Synthesis of Intermediate 11

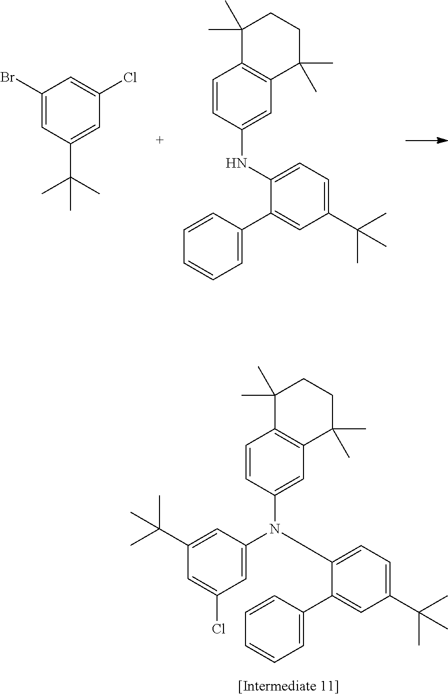 [SMILES: CC(C)(C)c1cc(Cl)cc(Br)c1 ; CC(C)(C)c1ccc(Nc2ccc3c(c2)C(C)(C)CCC3(C)C)c(-c2ccccc2)c1 ; CC(C)(C)c1cc(Cl)cc(N(c2ccc3c(c2)C(C)(C)CCC3(C)C)c2ccc(C(C)(C)C)cc2-c2ccccc2)c1]

From 40 g of 1-bromo-3-(tert-butyl)-5-chlorobenzene and 66.5 g of N-(5-(tert-butyl)-[1,1′-biphenyl]-2-yl)-5,5,8,8-tetramethyl-5,6,7,8-tetrahydronaphthalen-2-amine, Intermediate 11 (75 g) was obtained using the same material and equivalent weight as in the synthesis method of Intermediate 1 and using the same method as the synthesis method of Intermediate 1. (Yield 80%). MS[M+H]+=579

### 2) Synthesis of Intermediate 12

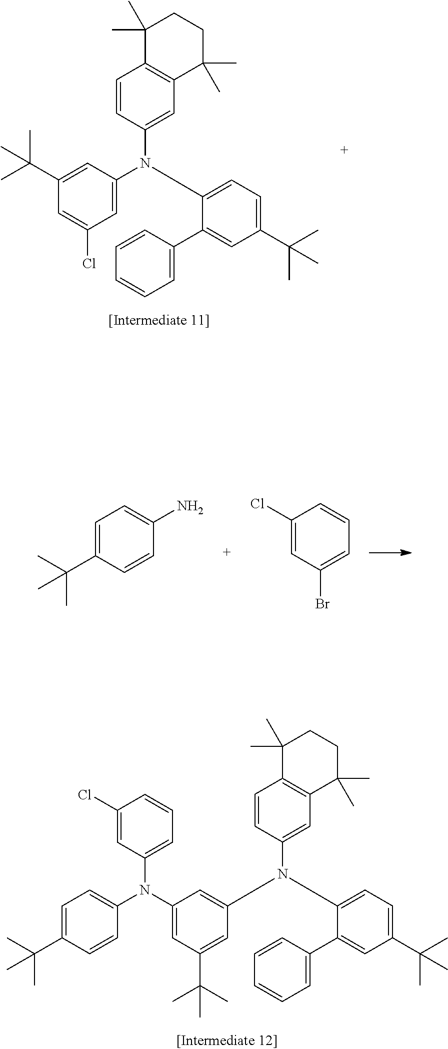

After Intermediate 11 (40 g), 11.3 g of 4-tert-butylaniline, 19.9 g of sodium-tert-butoxide, and 0.4 g of bis(tri-tert-butylphosphine)palladium(0) were put into 600 ml of toluene, the resulting mixture was refluxed for 1 hour, whether the reaction proceeded was confirmed, and then 13.2 g of 1-bromo-3-chlorobenzene was added thereto during the reflux reaction, and the reflux reaction was performed for an additional 1 hour. After the completion of the reaction, the resulting product was extracted, and then recrystallized to obtain Intermediate 12 (28.8 g). (Yield 52%). MS[M+H]+=801

### 3) Synthesis of Intermediate 13

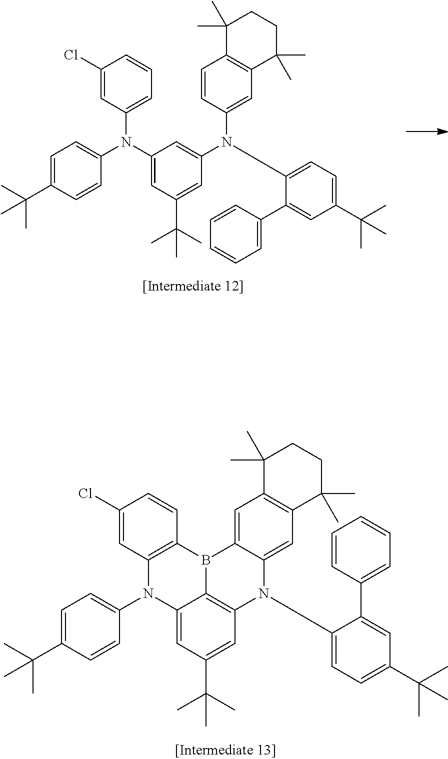

After Intermediate 12 (25 g) and 20.4 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Intermediate 13 (5.5 g) (yield 22%). MS[M+H]+=809

### 4) Synthesis of Compound 10

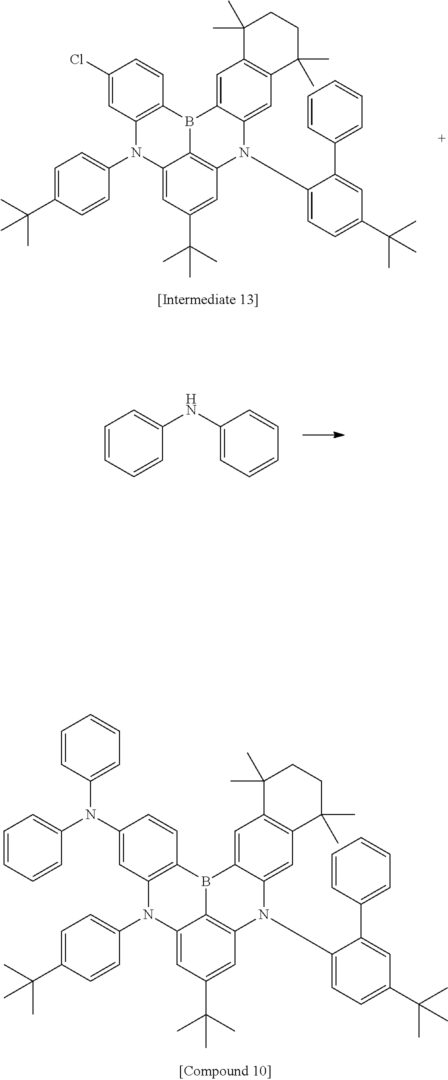

After Intermediate 13 (5 g), 1.5 g of diphenylamine, 1.2 g of sodium-tert-butoxide, and 0.05 g of bis(tri-tert-butylphosphine)palladium(0) were put into 80 ml of xylene, the resulting mixture was stirred under reflux for 5 hours. After the completion of the reaction, the resulting product was extracted, and then recrystallized to obtain Compound 10 (4.6 g). (Yield 80%). MS[M+H]+=943

### Synthesis Example 11. Synthesis of Compound 11

1) Synthesis of Intermediate 14

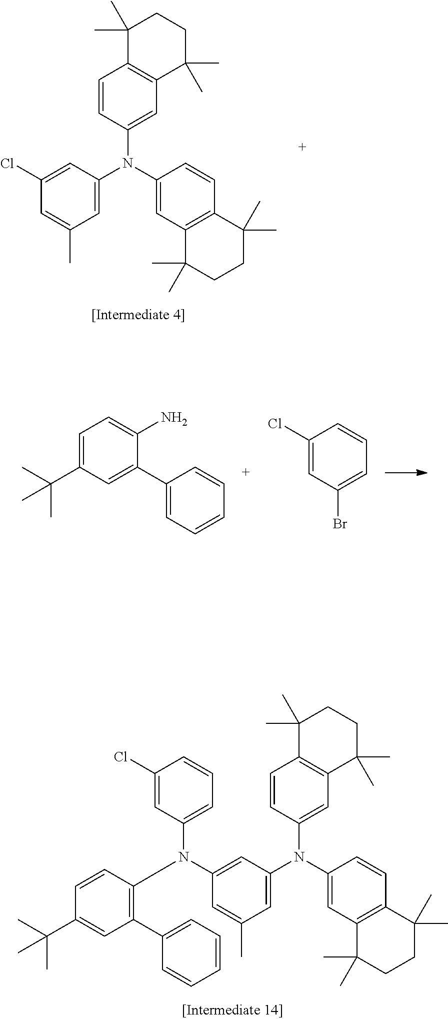

After Intermediate 4 (40 g), 17.6 g of 5-(tert-butyl)-[1,1′-biphenyl]-2-amine, 22.4 g of sodium-tert-butoxide, and 0.4 g of bis(tri-tert-butylphosphine)palladium(0) were put into 600 ml of toluene, the resulting mixture was refluxed for 1 hour, whether the reaction proceeded was confirmed, and then 14.9 g of 1-bromo-3-chlorobenzene was added thereto during the reflux reaction, and the reflux reaction was performed for an additional 1 hour. After the completion of the reaction, the resulting product was extracted, and then recrystallized to obtain Intermediate 14 (45 g). (Yield 71%). MS[M+H]+=814

### 2) Synthesis of Intermediate 15

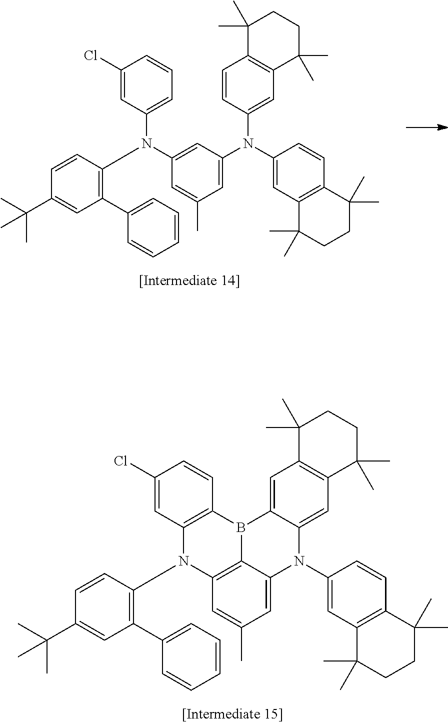

After Intermediate 14 (25 g) and 20.4 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Intermediate 15 (8.3 g) (yield 33%). MS[M+H]+=822

### 3) Synthesis of Compound 11

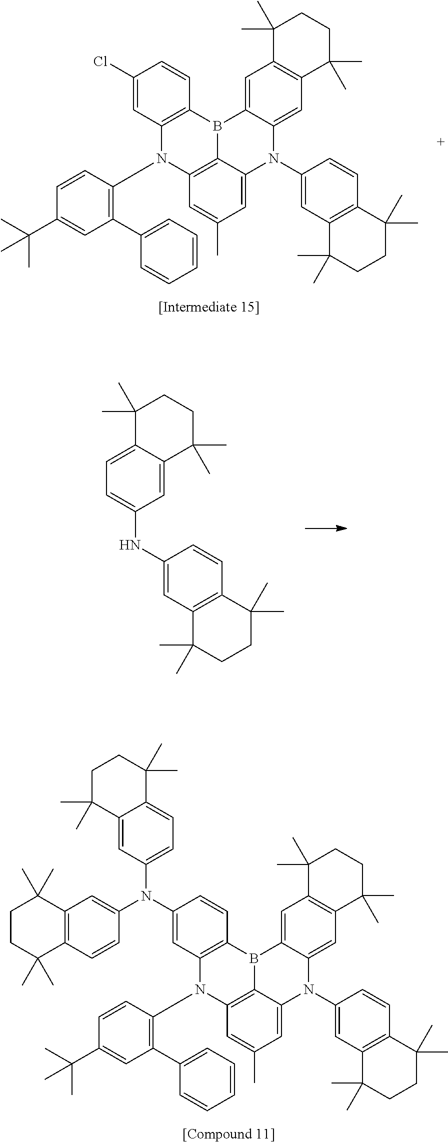

After Intermediate 15 (7 g), 3.4 g of bis(5,5,8,8-tetramethyl-5,6,7,8-tetrahydronaphthalen-2-yl)amine, 1.7 g of sodium-tert-butoxide, and 0.05 g of bis(tri-tert-butylphosphine)palladium(0) were put into 80 ml of xylene, the resulting mixture was stirred under reflux for 5 hours. After the completion of the reaction, the resulting product was extracted, and then recrystallized to obtain Compound 11 (7.4 g). (Yield 74%). MS[M+H]+=1175

### Synthesis Example 12. Synthesis of Compound 12

### 1) Synthesis of Intermediate 16

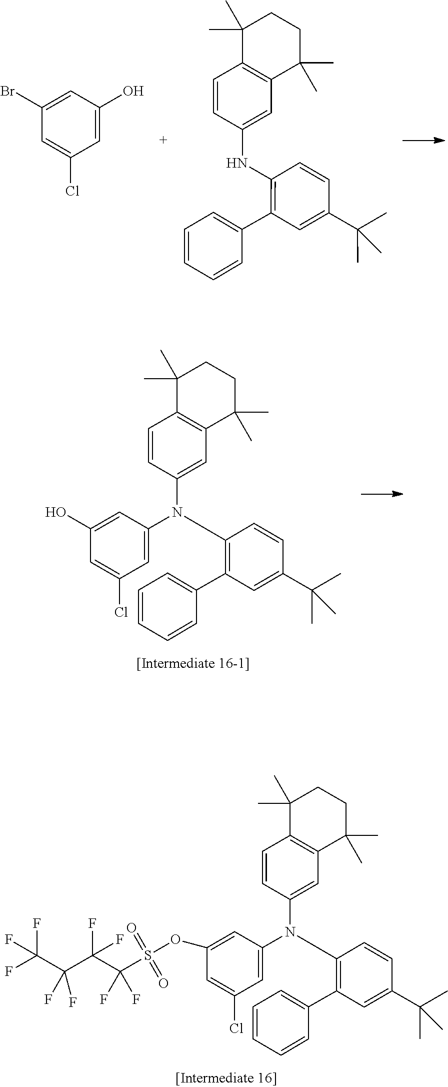 [SMILES: Oc1cc(Cl)cc(Br)c1 ; CC(C)(C)c1ccc(Nc2ccc3c(c2)C(C)(C)CCC3(C)C)c(-c2ccccc2)c1 ; CC(C)(C)c1ccc(N(c2cc(O)cc(Cl)c2)c2ccc3c(c2)C(C)(C)CCC3(C)C)c(-c2ccccc2)c1 ; CC(C)(C)c1ccc(N(c2cc(Cl)cc(OS(=O)(=O)C(F)(F)C(F)(F)C(F)(F)C(F)(F)F)c2)c2ccc3c(c2)C(C)(C)CCC3(C)C)c(-c2ccccc2)c1]

From 40 g of 3-bromo-5-chlorophenol and 79.4 g of N-(5-(tert-butyl)-[1,1′-biphenyl]-2-yl)-5,5,8,8-tetramethyl-5,6,7,8-tetrahydronaphthalen-2-amine, Intermediate 16-1 (70 g) was obtained through recrystallization using the same material and equivalent weight as in the synthesis method of Intermediate 1. (Yield 57%). MS[M+H]+=539

After Intermediate 16-1 (40 g), 20 ml of 1,1,2,2,3,3,4,4,4-nonafluorobutane-1-sulfonyl fluoride, and 30 g of potassium carbonate were put into a mixed solvent of 400 ml of tetrahydrofuran and 200 ml of water, the resulting mixture was reacted for 3 hours, and then the resulting product was extracted after the completion of the reaction, and then the solution was removed to obtain Intermediate 16 (58 g). (Yield 97%).

### 2) Synthesis of Intermediate 17

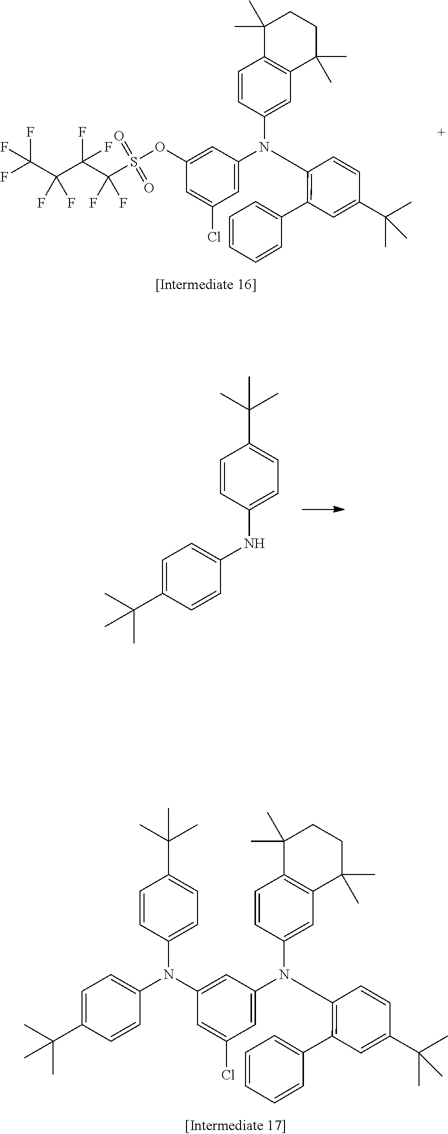

After Intermediate 16 (40 g), 14 g of bis(4-(tert-butyl)phenyl)amine, 0.85 g of tris(dibenzylidene-acetone)dipalladium(0) (Pd(dba)₂), 1.42 g of 2-dicyclo-hexylphosphino-2′,4′,6′-triisopropylbiphenyl (Xphos), and 48.6 g of cesium carbonate were put into 500 ml of xylene under a nitrogen atmosphere, the resulting mixture was stirred under reflux for 24 hours. After the completion of the reaction, the resulting product was extracted, and then recrystallized to obtain Intermediate 17 (31 g) (yield 78%). MS[M+H]+=802

### 3) Synthesis of Intermediate 18

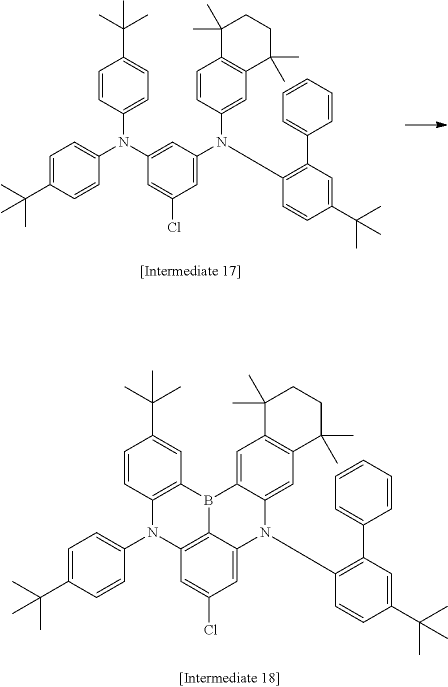

After Intermediate 17 (25 g) and 20.8 g of boron triiodide were put into 250 ml of 1,2-dichlorobenzene, the resulting mixture was stirred at 160° C. for 4 hours. After the completion of the reaction, the resulting product was extracted, and then recrystallized to obtain Intermediate 18 (7.9 g) (yield 31%). MS[M+H]+=810

### 4) Synthesis of Compound 12

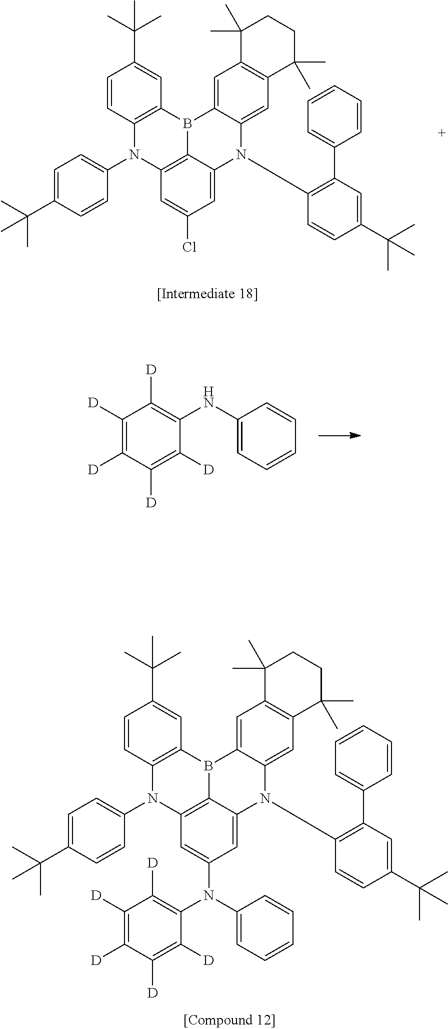

After Intermediate 18 (7 g), 1.5 g of diphenylamine-d5, 2.5 g of sodium-tert-butoxide, and 0.05 g of bis(tri-tert-butylphosphine)palladium(0) were put into 80 ml of xylene, the resulting mixture was stirred under reflux for 5 hours. After the completion of the reaction, the resulting product was extracted, and then recrystallized to obtain Compound 12 (6.2 g). (Yield 76%). MS[M+H]+=948

### Synthesis Example 13. Synthesis of Compound 13

### 1) Synthesis of Intermediate 19

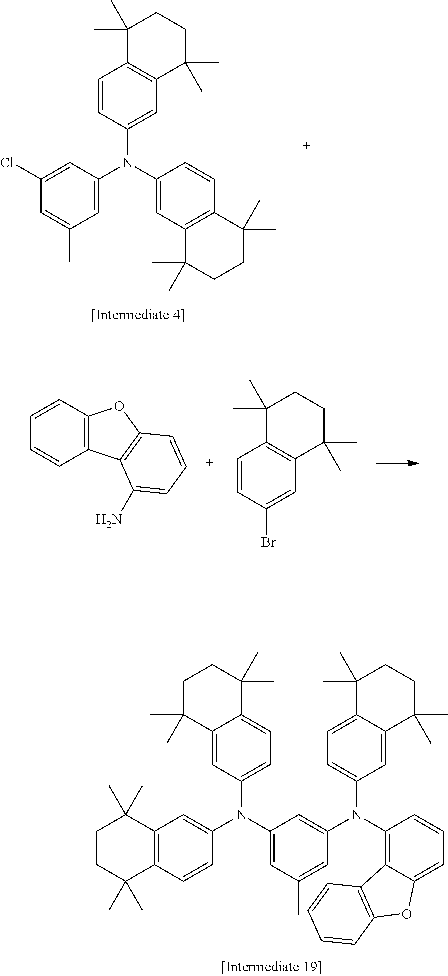

After Intermediate 4 (40 g), 14.3 g of dibenzo[b,d]furan-1-amine, 22.4 g of sodium-tert-butoxide, and 0.4 g of bis(tri-tert-butylphosphine)palladium(0) were put into 600 ml of toluene, the resulting mixture was refluxed for 1 hour, whether the reaction proceeded was confirmed, and then 20.8 g of 6-bromo-1,1,4,4-tetramethyl-1,2,3,4-tetrahydronaphthalene was added thereto during the reflux reaction, and the reflux reaction was performed for an additional 1 hour. After the completion of the reaction, the resulting product was extracted, and then recrystallized to obtain Intermediate 19 (54 g). (Yield 82%). MS[M+H]+=848

### 2) Synthesis of Compound 13

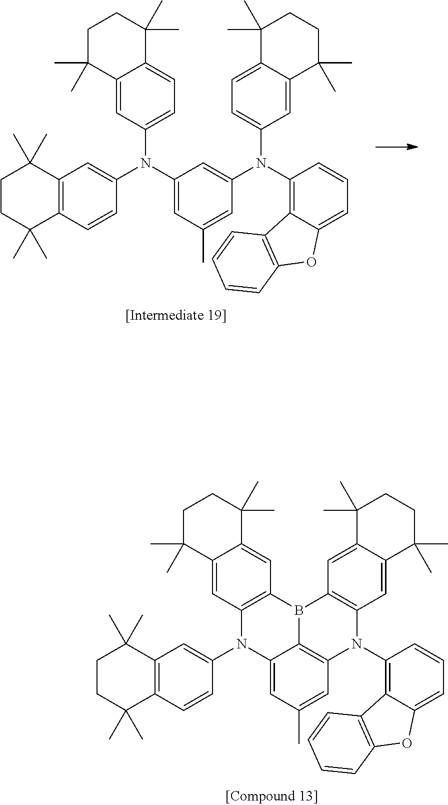 [SMILES: Cc1cc(N(c2ccc3c(c2)C(C)(C)CCC3(C)C)c2ccc3c(c2)C(C)(C)CCC3(C)C)cc(N(c2ccc3c(c2)C(C)(C)CCC3(C)C)c2cccc3oc4ccccc4c23)c1 ; Cc1cc2c3c(c1)N(c1cccc4oc5ccccc5c14)c1cc4c(cc1B3c1cc3c(cc1N2c1ccc2c(c1)C(C)(C)CCC2(C)C)C(C)(C)CCC3(C)C)C(C)(C)CCC4(C)C]

After Intermediate 19 (25 g) and 19.7 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Compound 13 (7.5 g)(yield 30%). MS [M+H]+=856

### Synthesis Example 14. Synthesis of Compound 14

### 1) Synthesis of Intermediate 20

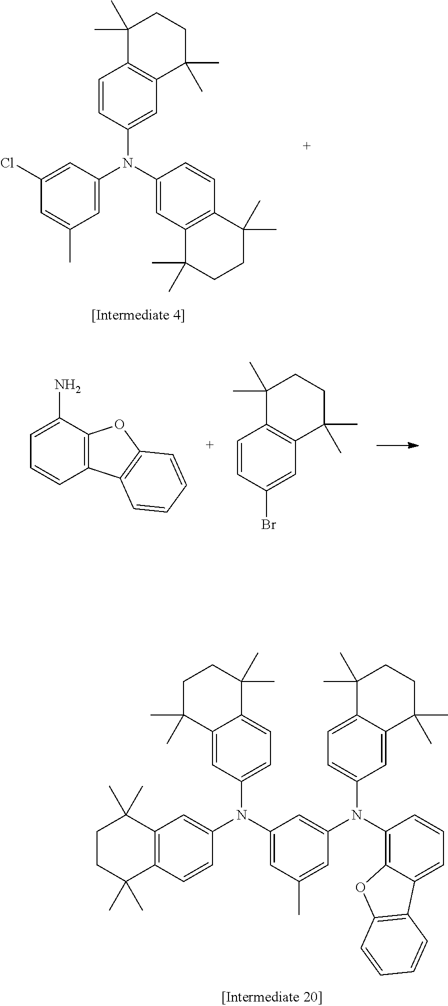

From Intermediate 4 (40 g) and 15.5 g of dibenzo[b,d]thiophen-4-amine, Intermediate 20 (54 g) was obtained using the same material and equivalent weight as in the synthesis method of Intermediate 19 and using the same method as the synthesis method of Intermediate 19. (Yield 78%). MS[M+H]+=864

### 2) Synthesis of Compound 14

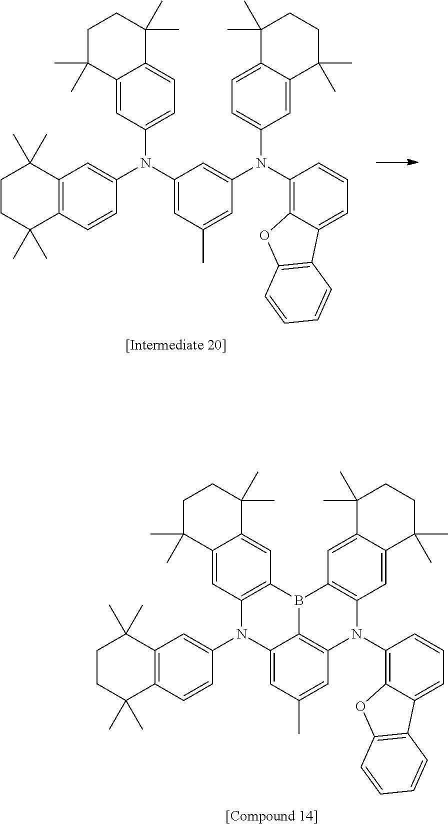 [SMILES: Cc1cc(N(c2ccc3c(c2)C(C)(C)CCC3(C)C)c2ccc3c(c2)C(C)(C)CCC3(C)C)cc(N(c2ccc3c(c2)C(C)(C)CCC3(C)C)c2cccc3c2oc2ccccc23)c1 ; Cc1cc2c3c(c1)N(c1cccc4c1oc1ccccc14)c1cc4c(cc1B3c1cc3c(cc1N2c1ccc2c(c1)C(C)(C)CCC2(C)C)C(C)(C)CCC3(C)C)C(C)(C)CCC4(C)C]

After Intermediate 20 (25 g) and 19.3 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Compound 14 (7.6 g) (yield 31%). MS[M+H]+=872

### Synthesis Example 15. Synthesis of Compound 15

### 1) Synthesis of Intermediate 21

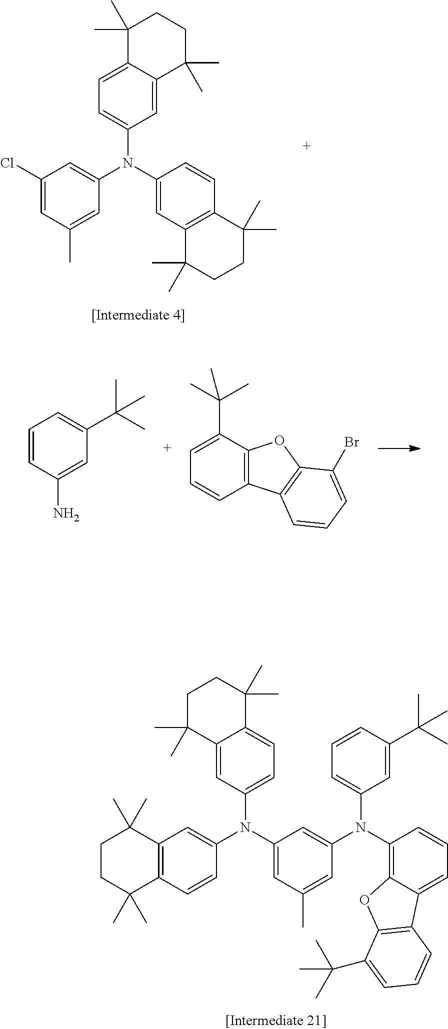 [SMILES: Cc1cc(Cl)cc(N(c2ccc3c(c2)C(C)(C)CCC3(C)C)c2ccc3c(c2)C(C)(C)CCC3(C)C)c1 ; CC(C)(C)c1cccc(N)c1 ; CC(C)(C)c1cccc2c1oc1c(Br)cccc12 ; Cc1cc(N(c2ccc3c(c2)C(C)(C)CCC3(C)C)c2ccc3c(c2)C(C)(C)CCC3(C)C)cc(N(c2cccc(C(C)(C)C)c2)c2cccc3c2oc2c(C(C)(C)C)cccc23)c1]

From Intermediate 4 (40 g), 18.1 g of dibenzo[b,d]furan-4-bromide, and 11.6 g of 3-(tert-butyl)aniline, Intermediate 21 (50 g) was obtained using the same material and equivalent weight as in the synthesis method of Intermediate 19 and using the same method as the synthesis method of Intermediate 19. (Yield 76%). MS[M+H]+=850

### 2) Synthesis of Compound 15

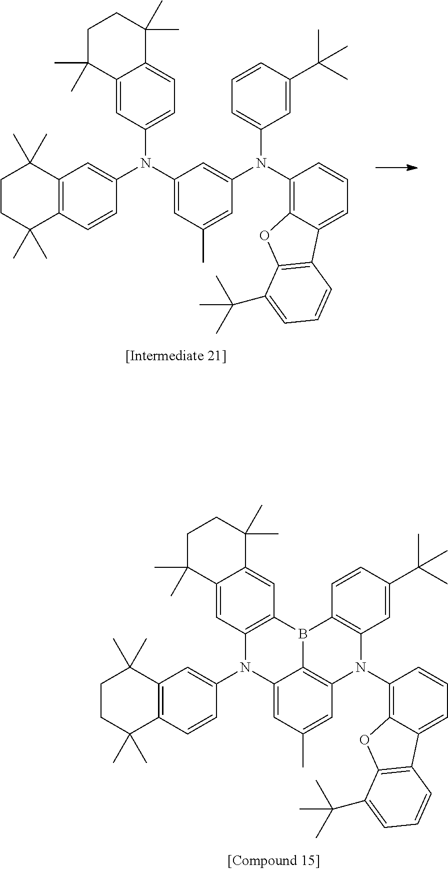 [SMILES: Cc1cc(N(c2ccc3c(c2)C(C)(C)CCC3(C)C)c2ccc3c(c2)C(C)(C)CCC3(C)C)cc(N(c2cccc(C(C)(C)C)c2)c2cccc3c2oc2c(C(C)(C)C)cccc23)c1 ; Cc1cc2c3c(c1)N(c1cccc4c1oc1c(C(C)(C)C)cccc14)c1cc(C(C)(C)C)ccc1B3c1cc3c(cc1N2c1ccc2c(c1)C(C)(C)CCC2(C)C)C(C)(C)CCC3(C)C]

After Intermediate 21 (25 g) and 21 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Compound 15 (8.3 g) (yield 31%). MS[M+H]+=858

### Synthesis Example 16. Synthesis of Compound 16

### 1) Synthesis of Intermediate 22

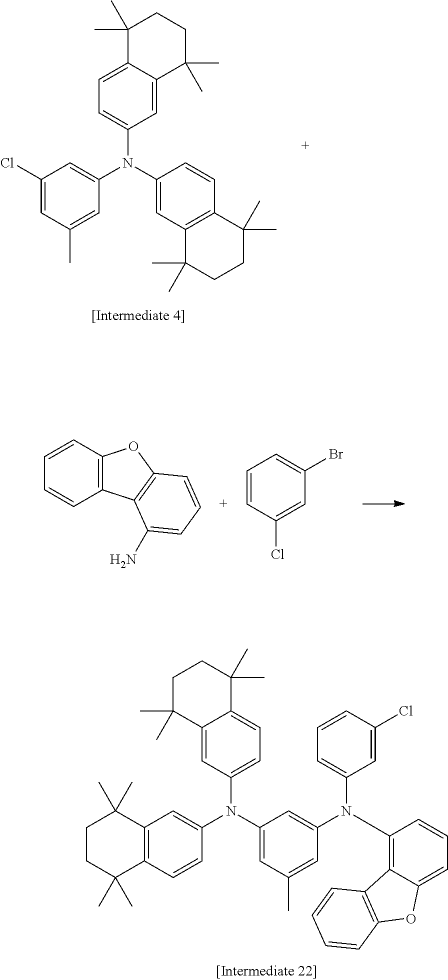 [SMILES: Cc1cc(Cl)cc(N(c2ccc3c(c2)C(C)(C)CCC3(C)C)c2ccc3c(c2)C(C)(C)CCC3(C)C)c1 ; Nc1cccc2oc3ccccc3c12 ; Clc1cccc(Br)c1 ; Cc1cc(N(c2ccc3c(c2)C(C)(C)CCC3(C)C)c2ccc3c(c2)C(C)(C)CCC3(C)C)cc(N(c2cccc(Cl)c2)c2cccc3oc4ccccc4c23)c1]

After Intermediate 4 (40 g), 14.3 g of dibenzo[b,d]furan-1-amine, 22.4 g of sodium-tert-butoxide, and 0.4 g of bis(tri-tert-butylphosphine)-palladium(0) were put into 600 ml of toluene, the resulting mixture was refluxed for 1 hour, whether the reaction proceeded was confirmed, and then 14.9 g of 1-bromo-3-chlorobenzene was added thereto during the reflux reaction, and the reflux reaction was performed for an additional 1 hour. After the completion of the reaction, the resulting product was extracted, and then recrystallized to obtain Intermediate 22 (46 g). (Yield 77%). MS[M+H]+=771

### 2) Synthesis of Intermediate 23

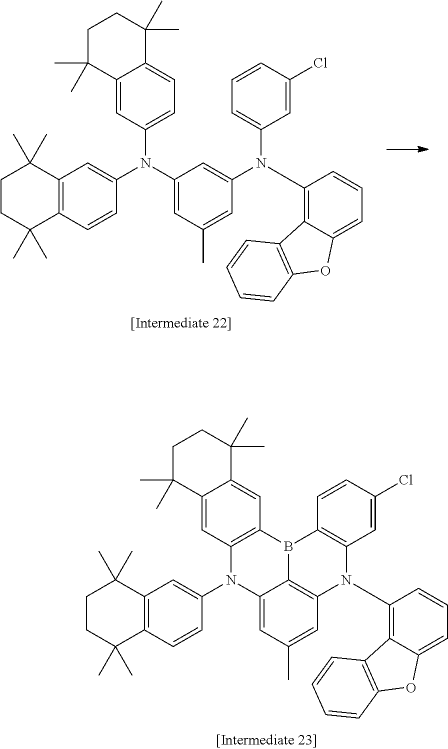 [SMILES: Cc1cc(N(c2ccc3c(c2)C(C)(C)CCC3(C)C)c2ccc3c(c2)C(C)(C)CCC3(C)C)cc(N(c2cccc(Cl)c2)c2cccc3oc4ccccc4c23)c1 ; Cc1cc2c3c(c1)N(c1cccc4oc5ccccc5c14)c1cc(Cl)ccc1B3c1cc3c(cc1N2c1ccc2c(c1)C(C)(C)CCC2(C)C)C(C)(C)CCC3(C)C]

After Intermediate 22 (25 g) and 21.6 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Intermediate 23 (7.8 g) (yield 31%). MS[M+H]+=780

### 3) Synthesis of Compound 16

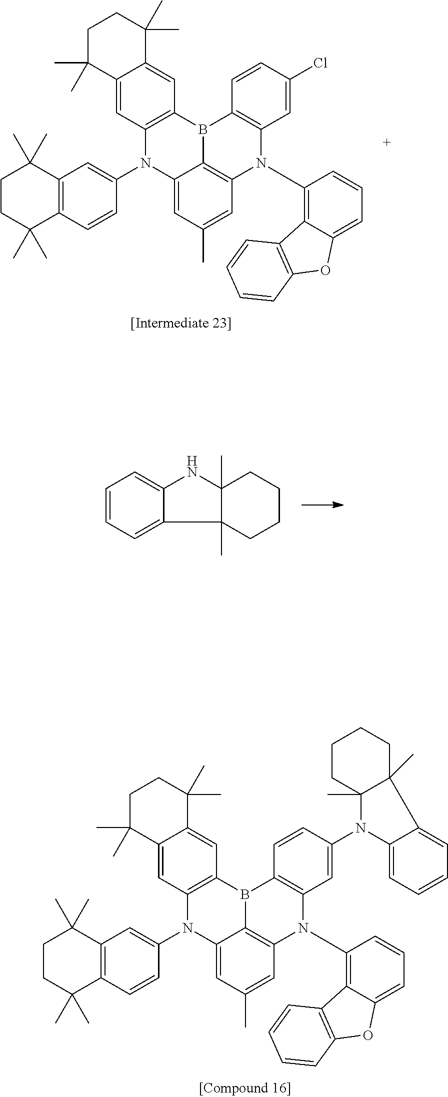 [SMILES: Cc1cc2c3c(c1)N(c1cccc4oc5ccccc5c14)c1cc(Cl)ccc1B3c1cc3c(cc1N2c1ccc2c(c1)C(C)(C)CCC2(C)C)C(C)(C)CCC3(C)C ; CC12CCCCC1(C)c1ccccc1N2 ; Cc1cc2c3c(c1)N(c1cccc4oc5ccccc5c14)c1cc(N4c5ccccc5C5(C)CCCCC45C)ccc1B3c1cc3c(cc1N2c1ccc2c(c1)C(C)(C)CCC2(C)C)C(C)(C)CCC3(C)C]

After Intermediate 23 (7 g), 1.8 g of 4a,9a-dimethyl-2,3,4,4a,9,9a-hexahydro-1H-carbazole, 1.7 g of sodium-tert-butoxide, and 0.05 g of bis(tri-tert-butylphosphine)palladium(0) were put into 80 ml of xylene, the resulting mixture was stirred under reflux for 5 hours. After the completion of the reaction, the resulting product was extracted, and then recrystallized to obtain Compound 16 (6.1 g). (Yield 72%). MS[M+H]+=945

### Synthesis Example 17. Synthesis of Compound 17

### 1) Synthesis of Intermediate 24

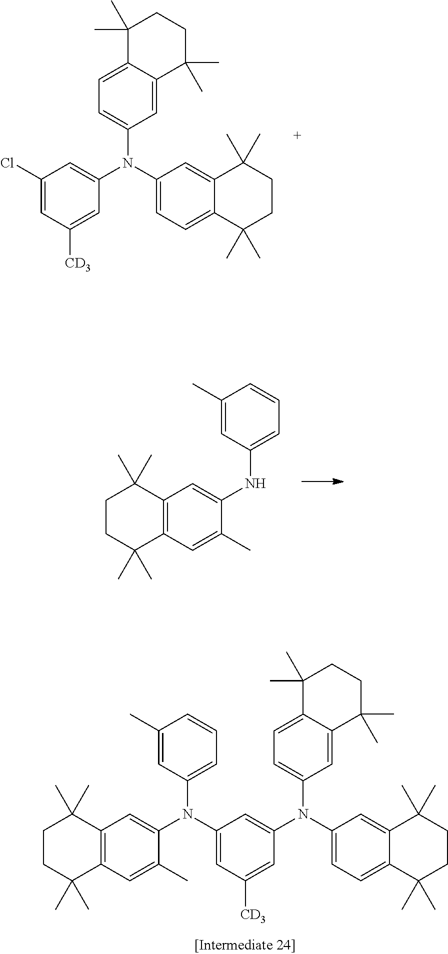 [SMILES: [2H]C([2H])([2H])c1cc(Cl)cc(N(c2ccc3c(c2)C(C)(C)CCC3(C)C)c2ccc3c(c2)C(C)(C)CCC3(C)C)c1 ; Cc1cccc(Nc2cc3c(cc2C)C(C)(C)CCC3(C)C)c1 ; [2H]C([2H])([2H])c1cc(N(c2ccc3c(c2)C(C)(C)CCC3(C)C)c2ccc3c(c2)C(C)(C)CCC3(C)C)cc(N(c2cccc(C)c2)c2cc3c(cc2C)C(C)(C)CCC3(C)C)c1]

From 30 g of N-(3-chloro-5-(methyl-d3)phenyl)-5,5,8,8-tetramethyl-N-(5,5,8,8-tetramethyl-5,6,7,8-tetrahydronapthalen-2-yl)-5,6,7,8-tetrahydronaphthalen-2-amine and 17.9 g of 3,5,5,8,8-pentamethyl-N-(m-tolyl)-5,6,7,8-tetrahydronaphthalen-2-amine, Intermediate 24 (31.3 g) was obtained using the same material and equivalent weight as in the synthesis method of Intermediate 2 and using the same method as the synthesis method of Intermediate 2. (Yield 69%). MS [M+H]+=789

### 2) Synthesis of Compound 17

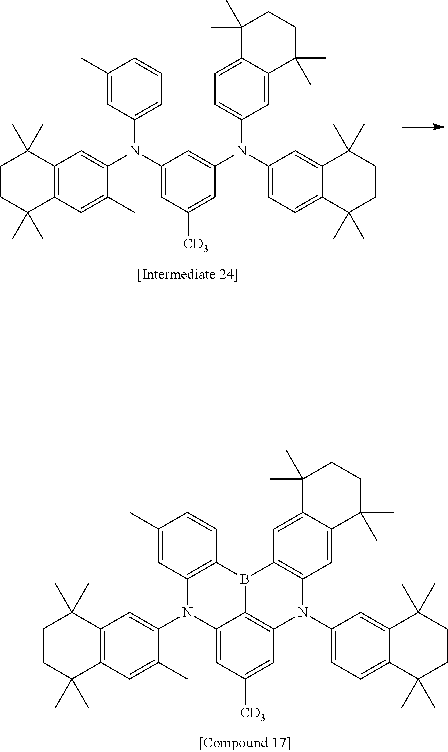 [SMILES: [2H]C([2H])([2H])c1cc(N(c2ccc3c(c2)C(C)(C)CCC3(C)C)c2ccc3c(c2)C(C)(C)CCC3(C)C)cc(N(c2cccc(C)c2)c2cc3c(cc2C)C(C)(C)CCC3(C)C)c1 ; [2H]C([2H])([2H])c1cc2c3c(c1)N(c1cc4c(cc1C)C(C)(C)CCC4(C)C)c1cc(C)ccc1B3c1cc3c(cc1N2c1ccc2c(c1)C(C)(C)CCC2(C)C)C(C)(C)CCC3(C)C]

After Intermediate 24 (25 g) and 20.4 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Compound 17 (7 g) (yield 29%). MS[M+H]+=797

### Synthesis Example 18. Synthesis of Compound 18

### 1) Synthesis of Intermediate 25

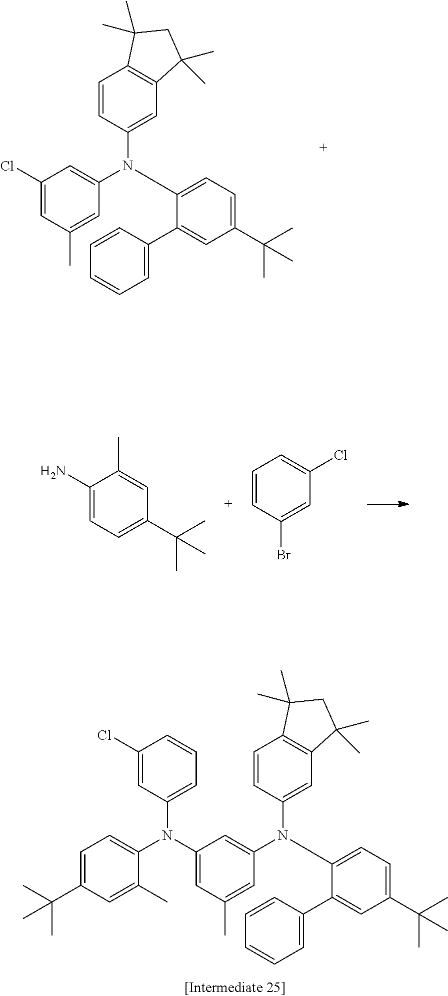

After 40 g of N-(5-(tert-butyl)-[1,1′-biphenyl]-2-yl)-N-(3-chloro-5-methylphenyl)-1,1,3,3-tetramethyl-2,3-dihydro-1H-inden-5-amine, 12.1 g of 4-(tert-butyl)-2-methylaniline, 22.1 g of sodium-tert-butoxide, and 0.4 g of bis(tri-tert-butylphosphine)palladium(0) were put into 600 ml of toluene, the resulting mixture was refluxed for 1 hour, whether the reaction proceeded was confirmed, and then 14.6 g of 1-bromo-3-chlorobenzene was added thereto during the reflux reaction, and the reflux reaction was performed for an additional 1 hour. After the completion of the reaction, the resulting product was extracted, and then recrystallized to obtain Intermediate 25 (43 g). (Yield 74%). MS[M+H]+=760

### 2) Synthesis of Intermediate 26

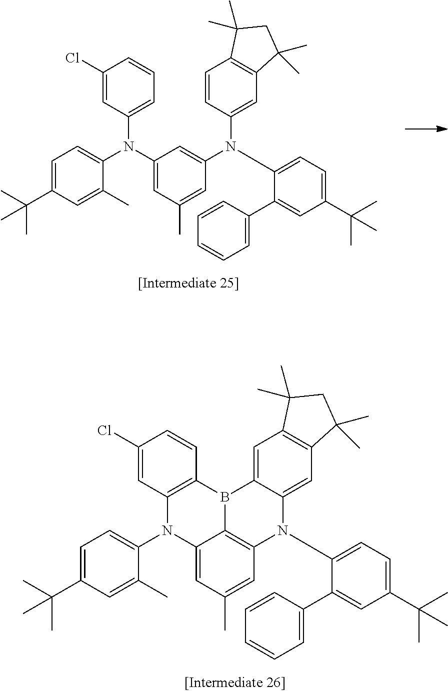

After Intermediate 25 (25 g) and 21.9 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Intermediate 26 (7.1 g) (yield 28%). MS[M+H]+=768

### 3) Synthesis of Compound 18

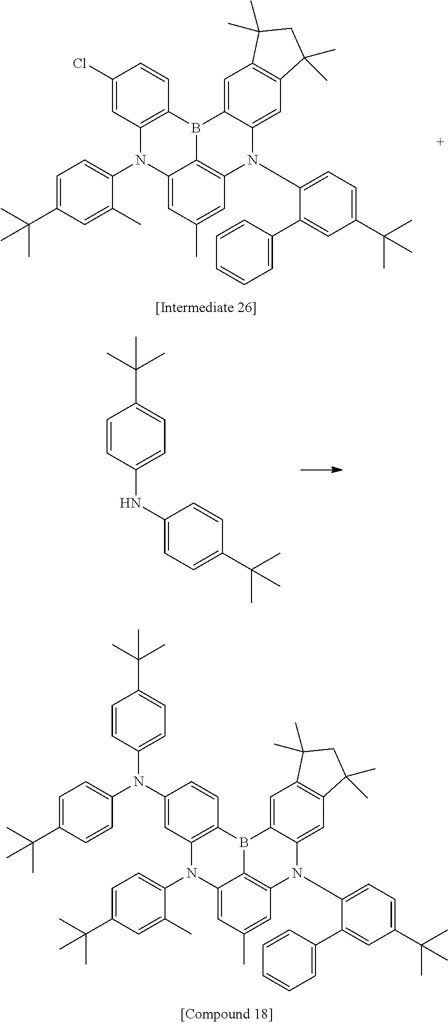

After Intermediate 26 (7 g), 2.5 g of bis (4-(tert-butyl)phenyl)amine, 1.7 g of sodium-tert-butoxide, and 0.05 g of bis(tri-tert-butylphosphine)palladium(0) were put into 80 ml of xylene, the resulting mixture was stirred under reflux for 5 hours. After the completion of the reaction, the resulting product was extracted, and then recrystallized to obtain Compound 18 (6.5 g). (Yield 72%). MS[M+H]+=1013

### Synthesis Example 19. Synthesis of Compound 19

### 1) Synthesis of Intermediate 28

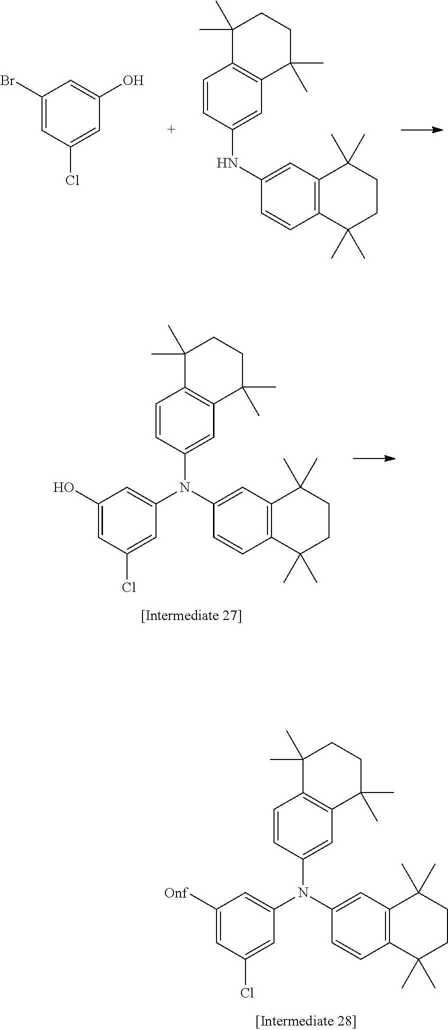

Here, Onf means a 1,1,2,2,3,3,4,4,4-nonafluorobutane-1-sulfonyl group.

From 40 g of 3-bromo-5-chlorophenol and 75.2 g of bis(5,5,8,8-tetramethyl-5,6,7,8-tetrahydronaphthalen-2-yl)amine, Intermediate 27 (70 g) was obtained using the same material and equivalent weight as in the synthesis method of Intermediate 35 and using the same method as the synthesis method of Intermediate 35. (Yield 70%). MS[M+H]+=517

From Intermediate 27 (40 g), Intermediate 28 (56 g) was obtained using the same material and equivalent weight as in the synthesis method of Intermediate 16 and using the same method as the synthesis method of Intermediate 16. (Yield 92%). MS[M+H]+=783

### 2) Synthesis of Intermediate 29

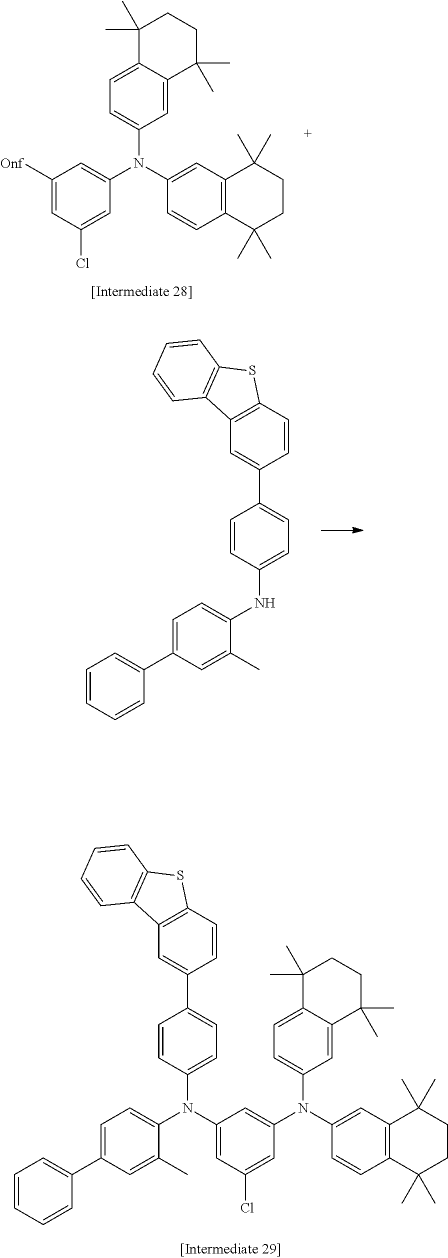

From Intermediate 28 (40 g) and 34 g of N-(4-(dibenzo[b,d]thiophen-2-yl)phenyl)-3-methyl-[1,1′-biphenyl]-4-amine, Intermediate 29 (54 g) was obtained using the same material and equivalent weight as in the synthesis method of Intermediate 17 and using the same method as the synthesis method of Intermediate 17. (Yield 74%). MS[M+H]+=940

### 3) Synthesis of Intermediate 30

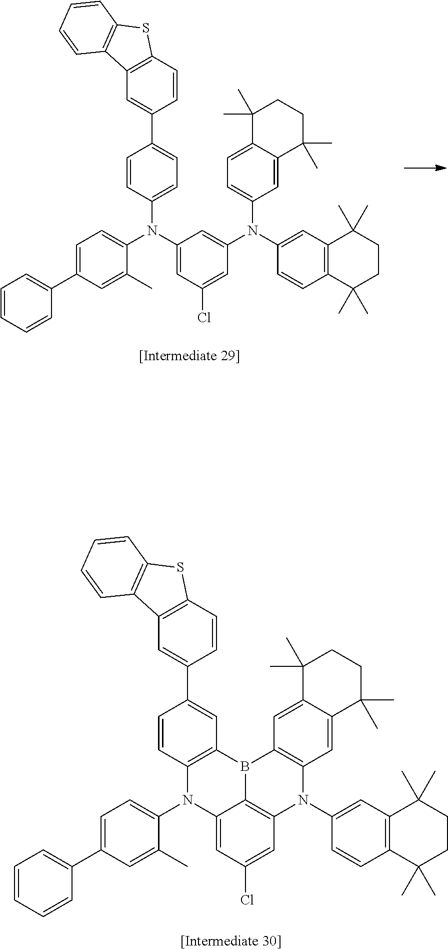 [SMILES: Cc1cc(-c2ccccc2)ccc1N(c1ccc(-c2ccc3sc4ccccc4c3c2)cc1)c1cc(Cl)cc(N(c2ccc3c(c2)C(C)(C)CCC3(C)C)c2ccc3c(c2)C(C)(C)CCC3(C)C)c1 ; Cc1cc(-c2ccccc2)ccc1N1c2ccc(-c3ccc4sc5ccccc5c4c3)cc2B2c3cc4c(cc3N(c3ccc5c(c3)C(C)(C)CCC5(C)C)c3cc(Cl)cc1c32)C(C)(C)CCC4(C)C]

After Intermediate 29 (25 g) and 17.7 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Intermediate 30 (7.5 g) (yield 30%). MS[M+H]+=948

### 3) Synthesis of Compound 19

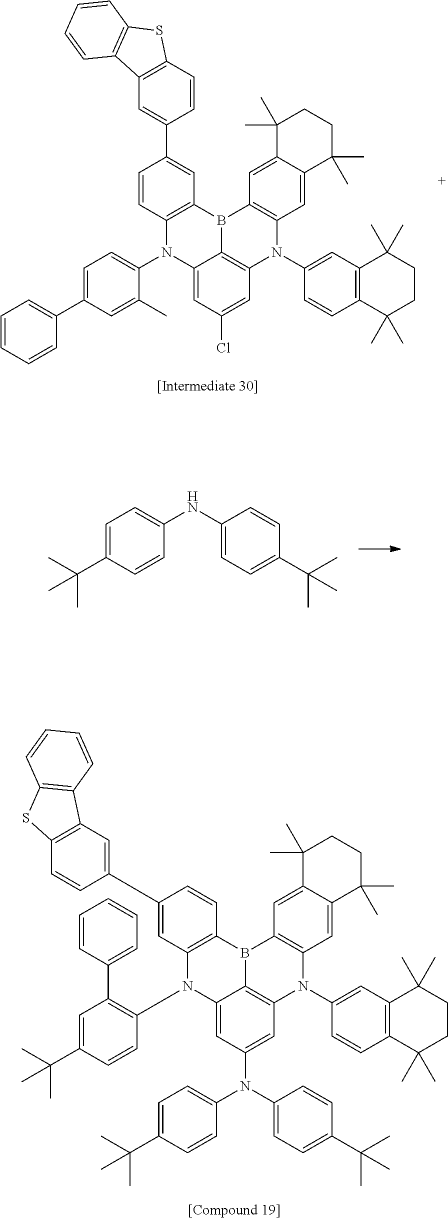

After Intermediate 30 (7 g), 2.1 g of bis (4-(tert-butyl)phenyl)amine, 1.7 g of sodium-tert-butoxide, and 0.05 g of bis(tri-tert-butylphosphine)palladium(0) were put into 80 ml of xylene, the resulting mixture was stirred under reflux for 5 hours. After the completion of the reaction, the resulting product was extracted, and then recrystallized to obtain Compound 19 (6.6 g). (Yield 72%). MS[M+H]+=1235

### Synthesis Example 20. Synthesis of Compound 20

### 1) Synthesis of Intermediate 31

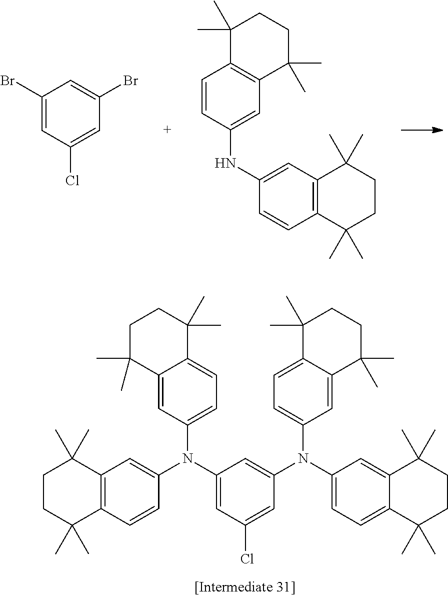 [SMILES: Clc1cc(Br)cc(Br)c1 ; CC1(C)CCC(C)(C)c2cc(Nc3ccc4c(c3)C(C)(C)CCC4(C)C)ccc21 ; CC1(C)CCC(C)(C)c2cc(N(c3cc(Cl)cc(N(c4ccc5c(c4)C(C)(C)CCC5(C)C)c4ccc5c(c4)C(C)(C)CCC5(C)C)c3)c3ccc4c(c3)C(C)(C)CCC4(C)C)ccc21]

From 40 g of 1,3-dibromo-5-chlorobenzene and 115.3 g of bis(5,5,8,8-tetramethyl-5,6,7,8-tetrahydronaphthalen-2-yl)amine under a nitrogen atmosphere, Intermediate 31 (99 g) was obtained using the same material and equivalent weight as in the synthesis method of Intermediate 9 and using the same method as the synthesis method of Intermediate 9. (Yield 75%). MS[M+H]+=888

### 2) Synthesis of Intermediate 32

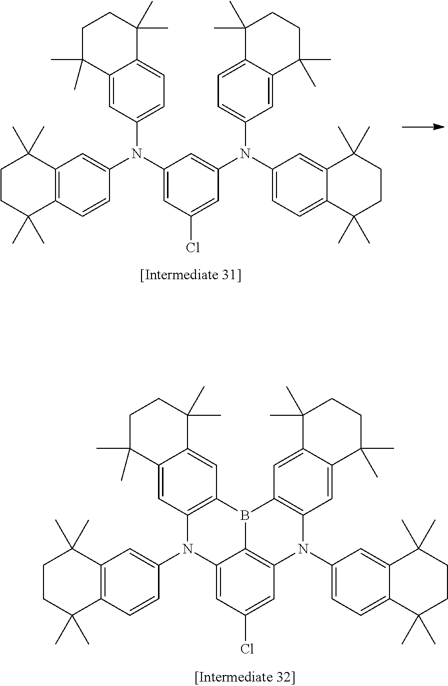 [SMILES: CC1(C)CCC(C)(C)c2cc(N(c3cc(Cl)cc(N(c4ccc5c(c4)C(C)(C)CCC5(C)C)c4ccc5c(c4)C(C)(C)CCC5(C)C)c3)c3ccc4c(c3)C(C)(C)CCC4(C)C)ccc21 ; CC1(C)CCC(C)(C)c2cc(N3c4cc5c(cc4B4c6cc7c(cc6N(c6ccc8c(c6)C(C)(C)CCC8(C)C)c6cc(Cl)cc3c64)C(C)(C)CCC7(C)C)C(C)(C)CCC5(C)C)ccc21]

After Intermediate 31 (25 g) and 18.7 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Intermediate 32 (7.7 g) (yield 31%). MS[M+H]+=896

### 3) Synthesis of Compound 20

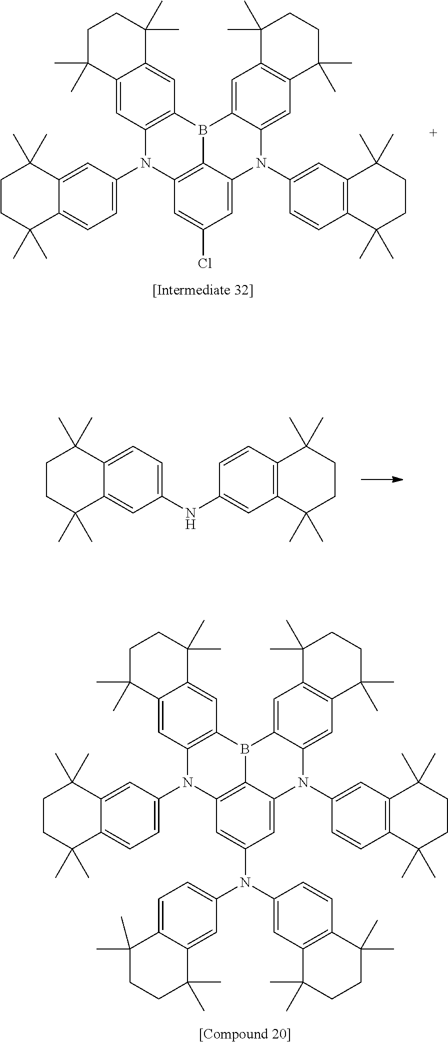 [SMILES: CC1(C)CCC(C)(C)c2cc(N3c4cc5c(cc4B4c6cc7c(cc6N(c6ccc8c(c6)C(C)(C)CCC8(C)C)c6cc(Cl)cc3c64)C(C)(C)CCC7(C)C)C(C)(C)CCC5(C)C)ccc21 ; CC1(C)CCC(C)(C)c2cc(Nc3ccc4c(c3)C(C)(C)CCC4(C)C)ccc21 ; CC1(C)CCC(C)(C)c2cc(N(c3cc4c5c(c3)N(c3ccc6c(c3)C(C)(C)CCC6(C)C)c3cc6c(cc3B5c3cc5c(cc3N4c3ccc4c(c3)C(C)(C)CCC4(C)C)C(C)(C)CCC5(C)C)C(C)(C)CCC6(C)C)c3ccc4c(c3)C(C)(C)CCC4(C)C)ccc21]

After Intermediate 32 (7 g), 3 g of bis(5,5,8,8-tetramethyl-5,6,7,8-tetrahydronaphthalen-2-yl)amine, 1.5 g of sodium-tert-butoxide, and 0.04 g of bis(tri-tert-butylphosphine)palladium(0) were put into 80 ml of xylene, the resulting mixture was stirred under reflux for 5 hours. After the completion of the reaction, the resulting product was extracted, and then recrystallized to obtain Compound 20 (7.1 g). (Yield 73%). MS[M+H]+=

### Synthesis Example 21. Synthesis of Compound 21

### 1) Synthesis of Intermediate 33

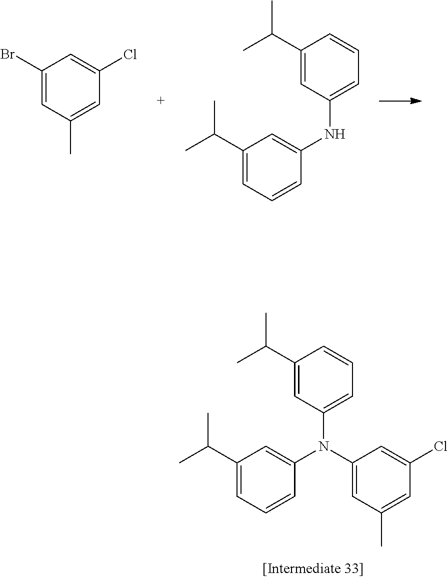

Intermediate 33 (25 g) was obtained using 25 g of bis(3-isopropylphenyl)amine in the same manner as in the method of synthesizing Intermediate 1 of Synthesis Example 1. (Yield 67%). MS[M+H]+=378

### 2) Synthesis of Intermediate 34

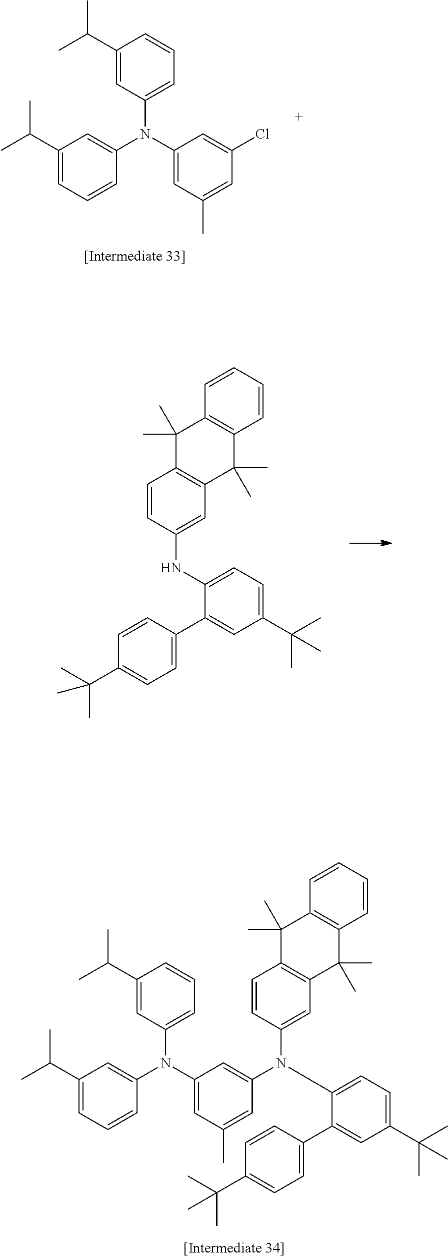 [SMILES: Cc1cc(Cl)cc(N(c2cccc(C(C)C)c2)c2cccc(C(C)C)c2)c1 ; CC(C)(C)c1ccc(-c2cc(C(C)(C)C)ccc2Nc2ccc3c(c2)C(C)(C)c2ccccc2C3(C)C)cc1 ; Cc1cc(N(c2cccc(C(C)C)c2)c2cccc(C(C)C)c2)cc(N(c2ccc3c(c2)C(C)(C)c2ccccc2C3(C)C)c2ccc(C(C)(C)C)cc2-c2ccc(C(C)(C)C)cc2)c1]

Intermediate 34 (32.2 g) was obtained using 27.3 g of N-(5-(tert-butyl)-[1,1′-biphenyl]-2-yl)-9,9,10,10-tetramethyl-9,10-dihydroanthracen-2-amine in the same manner as in the method of synthesizing Intermediate 2 of Synthesis Example 1. (Yield 71%). MS[M+H]+=858

### 3) Synthesis of Compound 21

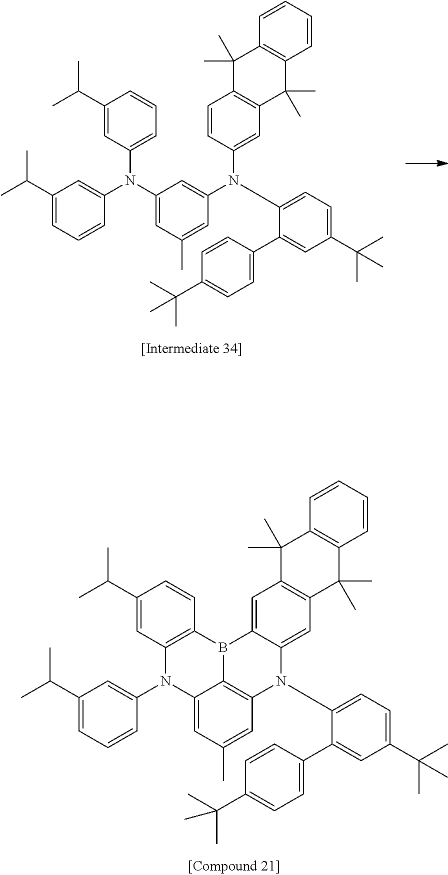

After Intermediate 34 (25 g) and 20.1 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Compound 21 (7.3 g) (yield 29%). MS[M+H]+=866

### Synthesis Example 22. Synthesis of Compound 22

### 1) Synthesis of Compound 22

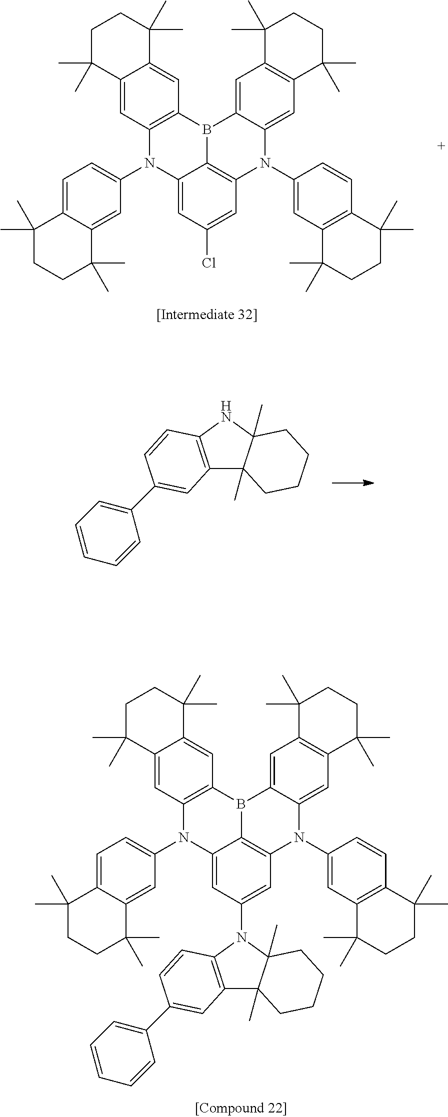

After Intermediate 32 (7 g), 1.93 g of 4a,9a-dimethyl-6-phenyl-2,3,4,4a,9,9a-hexahydro-1H-carbazole, 1.52 g of sodium-tert-butoxide, and 0.04 g of bis(tri-tert-butylphosphine)palladium(0) were put into 80 ml of xylene under a nitrogen atmosphere, the resulting mixture was stirred under reflux for 5 hours. After the completion of the reaction, the resulting product was extracted, and then recrystallized to obtain Compound 22 (6.9 g). (Yield 78%). MS[M+H]+=1137

### Synthesis Example 23. Synthesis of Compound 23

### 1) Synthesis of Intermediate 36

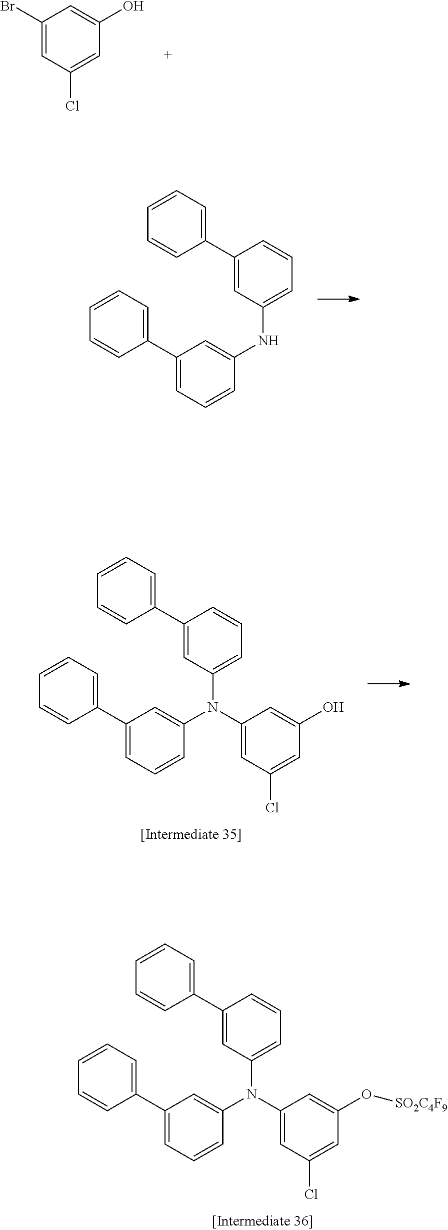

Intermediate 36 (40.5 g) was obtained using 35 g of di([1,1′-biphenyl]-3-yl)amine in the same manner as in the method of synthesizing Intermediate 16 of Synthesis Example 12. (Yield: 51%).

### 2) Synthesis of Intermediate 37

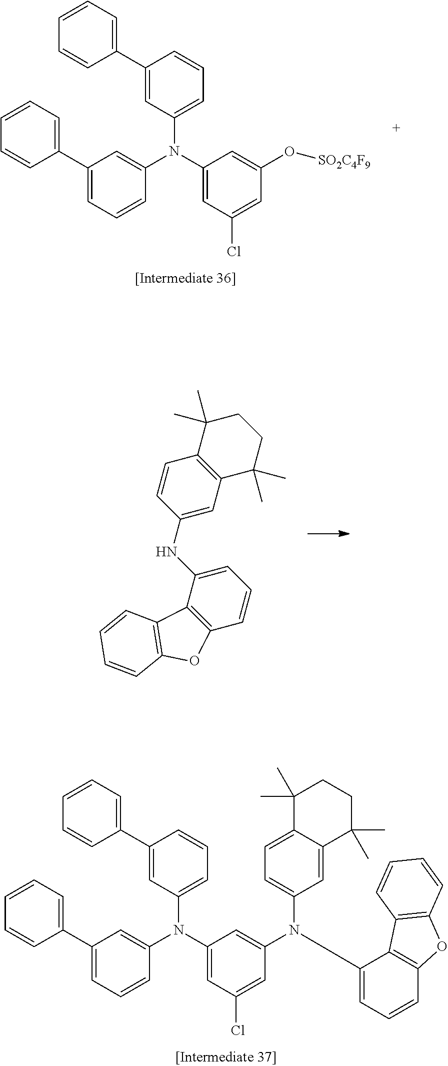

Intermediate 37 (31.8 g) was obtained using 20.4 g of N-(5,5,8,8-tetramethyl-5,6,7,8-tetrahydronaphthalen-2-yl)dibenzo[b,d]furan-1-amine in the same manner as in the method of synthesizing Intermediate 17 of Synthesis Example 12. (Yield 72%). MS[M+H]+=799

### 3) Synthesis of Intermediate 38

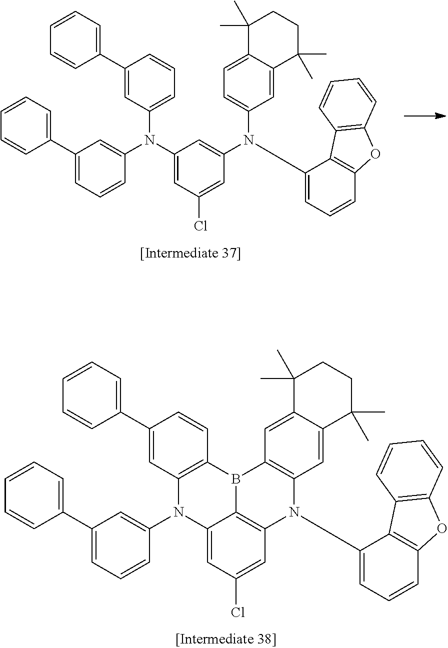 [SMILES: CC1(C)CCC(C)(C)c2cc(N(c3cc(Cl)cc(N(c4cccc(-c5ccccc5)c4)c4cccc(-c5ccccc5)c4)c3)c3cccc4oc5ccccc5c34)ccc21 ; CC1(C)CCC(C)(C)c2cc3c(cc21)B1c2ccc(-c4ccccc4)cc2N(c2cccc(-c4ccccc4)c2)c2cc(Cl)cc(c21)N3c1cccc2oc3ccccc3c12]

Intermediate 38 (11.8 g) was obtained using Intermediate 37 (30 g) in the same manner as in the method of synthesizing Intermediate 18 of Synthesis Example 12. (Yield 39%). MS[M+H]+=808

### 3) Synthesis of Compound 23

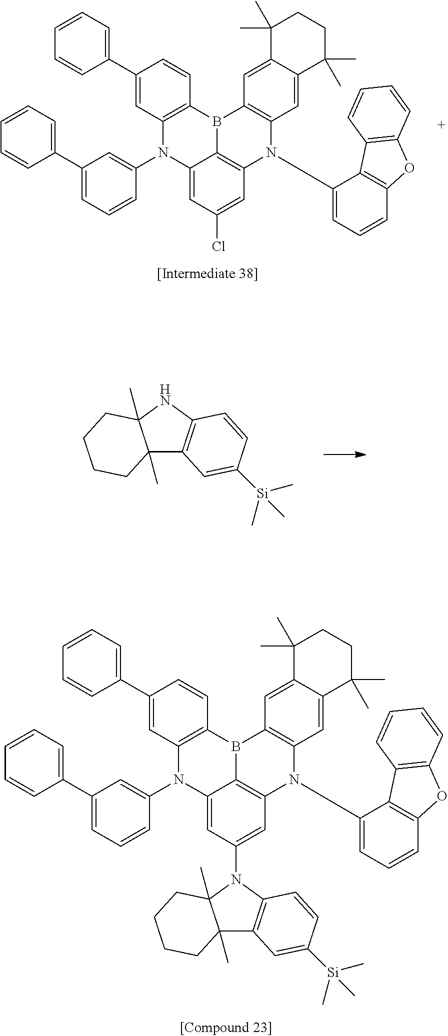 [SMILES: CC1(C)CCC(C)(C)c2cc3c(cc21)B1c2ccc(-c4ccccc4)cc2N(c2cccc(-c4ccccc4)c2)c2cc(Cl)cc(c21)N3c1cccc2oc3ccccc3c12 ; CC12CCCCC1(C)c1cc([Si](C)(C)C)ccc1N2 ; CC1(C)CCC(C)(C)c2cc3c(cc21)B1c2ccc(-c4ccccc4)cc2N(c2cccc(-c4ccccc4)c2)c2cc(N4c5ccc([Si](C)(C)C)cc5C5(C)CCCCC45C)cc(c21)N3c1cccc2oc3ccccc3c12]

Compound 23 (6.2 g) was obtained using Intermediate 38 (7 g) and 2.4 g of 4a,9a-dimethyl-6-(trimethylsilyl)-2,3,4,4a,9,9a-hexahydro-1H-carbazole in the same manner as in the method of synthesizing Compound 12 of Synthesis Example 12. (Yield 69%). MS[M+H]+=1045

### Synthesis Example 24. Synthesis of Compound 24

### 1) Synthesis of Intermediate 39

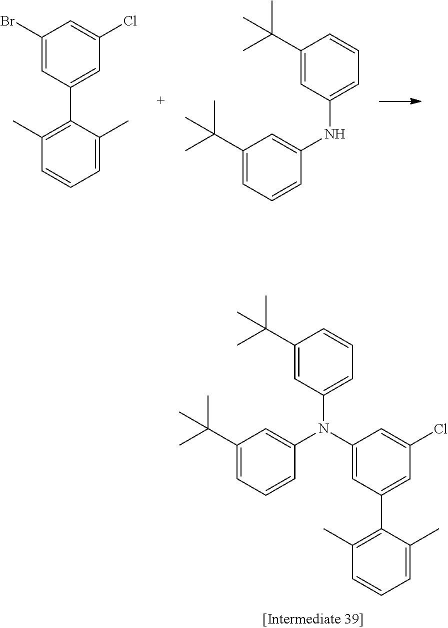 [SMILES: Cc1cccc(C)c1-c1cc(Cl)cc(Br)c1 ; CC(C)(C)c1cccc(Nc2cccc(C(C)(C)C)c2)c1 ; Cc1cccc(C)c1-c1cc(Cl)cc(N(c2cccc(C(C)(C)C)c2)c2cccc(C(C)(C)C)c2)c1]

Intermediate 39 (27.3 g) was obtained using 25 g of 3′-bromo-5′-chloro-2,6-dimethyl-1,1′-biphenyl and 23.8 g of bis(3-(tert-butyl)phenyl)amine in the same manner as in the method of synthesizing Intermediate 1 of Synthesis Example 1. (Yield 65%). MS[M+H]+=496

### 2) Synthesis of Intermediate 40

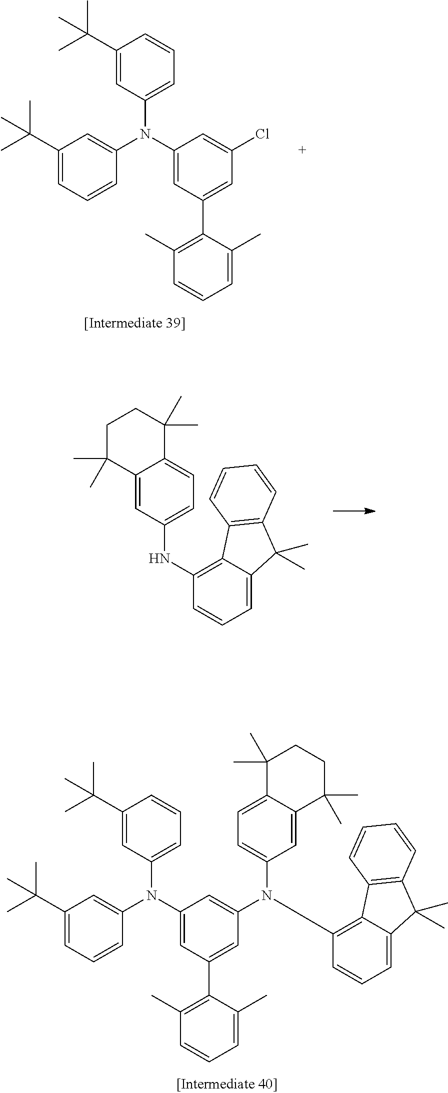

Intermediate 40 (23.9 g) was obtained using Intermediate 39 (22 g) and 17.5 g of 9,9-dimethyl-N-(5,5,8,8-tetramethyl-5,6,7,8-tetrahydronaphthalen-2-yl)-9H-fluoren-4-amine in the same manner as in the method of synthesizing Intermediate 2 of Synthesis Example 1. (Yield 63%). MS[M+H]+=856

### 3) Synthesis of Compound 24

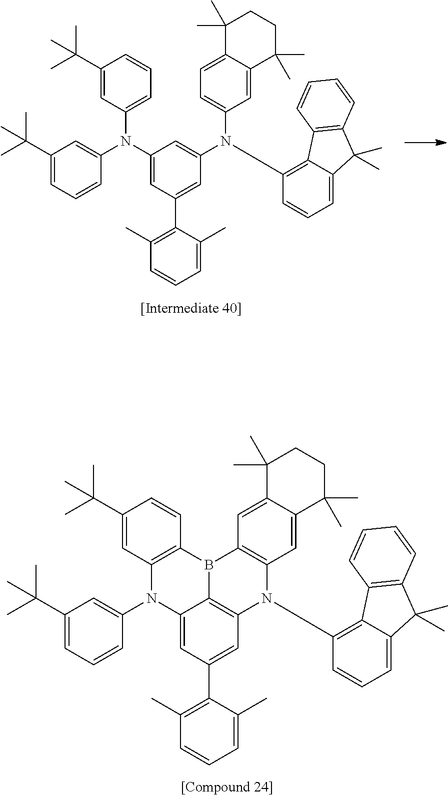

Intermediate 24 (4.2 g) was obtained using Intermediate 39 (20 g) in the same manner as in the method of synthesizing Compound 11 of Synthesis Example 1. (Yield 21%). MS[M+H]+=864

### Synthesis Example 25. Synthesis of Compound 26

### 1) Synthesis of Compound 26

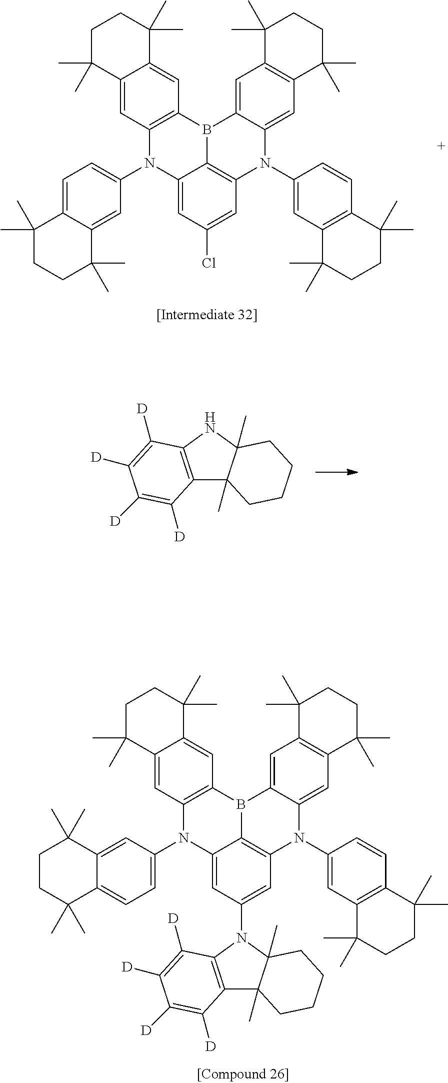

After Intermediate 4 (7 g), 1.6 g of 4a,9a-dimethyl-2,3,4,4a,9,9a-hexahydro-1H-carbazole-5,6,7,8-d4, 1.6 g of sodium-tert-butoxide, and 0.04 g of bis(tri-tert-butylphosphine)palladium(0) were put into 100 ml of toluene under a nitrogen atmosphere, the resulting mixture was stirred under reflux for 6 hours. After the completion of the reaction, the resulting product was extracted, and then recrystallized to obtain Compound 26 (6.5 g). (Yield 78%). MS[M+H]+=1065

### Synthesis Example 26. Synthesis of Compound 25

### 1) Synthesis of Intermediate 41

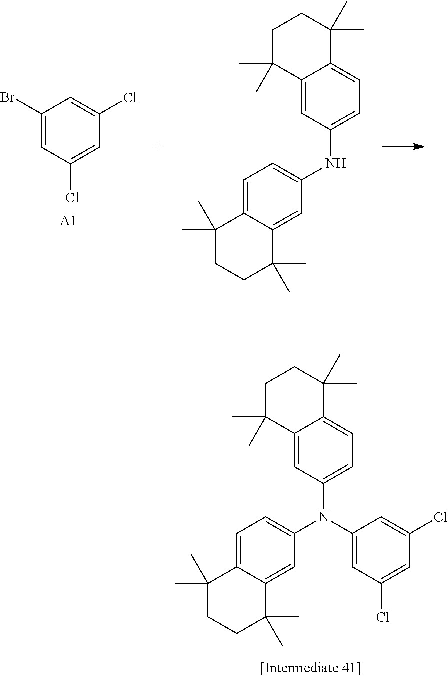

After A1 (40 g), 69 g of bis(5,5,8,8-tetramethyl-5,6,7,8-tetrahydronaphthalen-2-yl)amine, 34.1 g of sodium-tert-butoxide, and 0.9 g of bis(tri-tert-butylphosphine)palladium(0) were put into 600 ml of toluene under a nitrogen atmosphere, the resulting mixture was stirred under reflux for 2 hours. After the completion of the reaction, the resulting product was extracted, and then recrystallized to obtain Intermediate 41 (70 g). (Yield 74%). MS[M+H]+=535

### 2) Synthesis of Intermediate 42

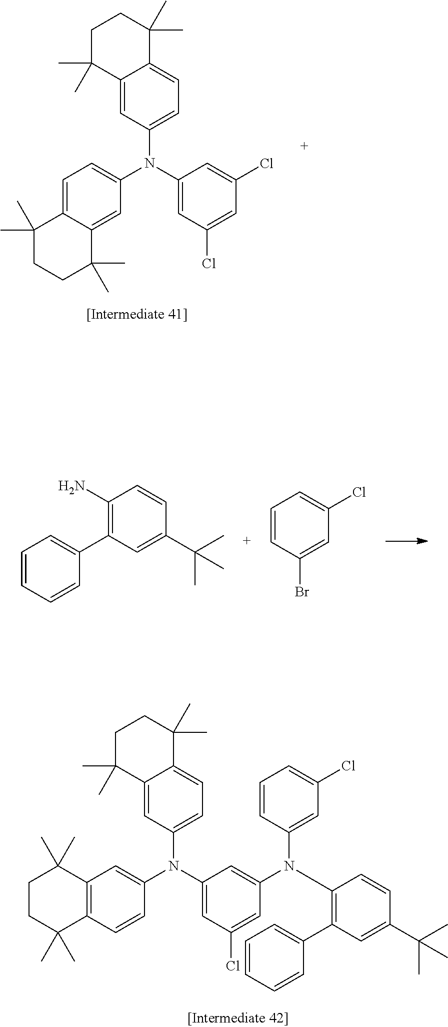 [SMILES: CC1(C)CCC(C)(C)c2cc(N(c3cc(Cl)cc(Cl)c3)c3ccc4c(c3)C(C)(C)CCC4(C)C)ccc21 ; CC(C)(C)c1ccc(N)c(-c2ccccc2)c1 ; Clc1cccc(Br)c1 ; CC(C)(C)c1ccc(N(c2cccc(Cl)c2)c2cc(Cl)cc(N(c3ccc4c(c3)C(C)(C)CCC4(C)C)c3ccc4c(c3)C(C)(C)CCC4(C)C)c2)c(-c2ccccc2)c1]

After Intermediate 41 (40 g), 16.9 g of 5-(tert-butyl)-[1,1′-biphenyl]-2-amine, 0.4 g of bis(tri-tert-butylphosphine)palladium(0), and 18 g of sodium-tert-butoxide were put into 600 ml of toluene under a nitrogen atmosphere, the resulting mixture was stirred under reflux for 1 hour. Thereafter, whether the reaction proceeded was confirmed, and then 14.3 g of 1-bromo-3-chlorobenzene was introduced thereinto during the stirring, and then the resulting mixture was stirred under reflux for 4 hours. After the completion of the reaction, the resulting product was extracted, and then recrystallized to obtain Intermediate 42 (45 g). (Yield 72%). MS[M+H]+=835

### 3) Synthesis of Intermediate 43

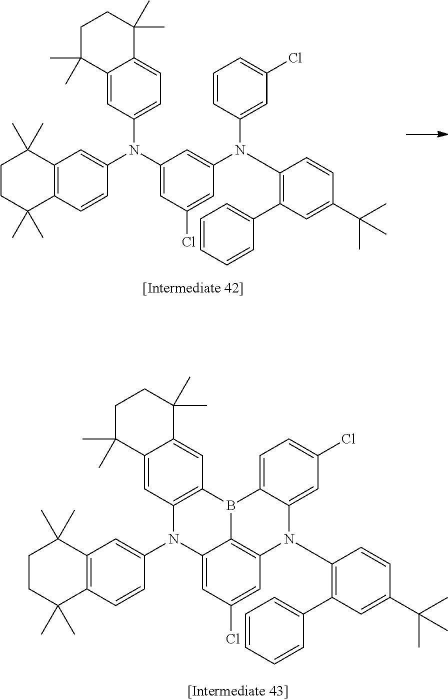 [SMILES: CC(C)(C)c1ccc(N(c2cccc(Cl)c2)c2cc(Cl)cc(N(c3ccc4c(c3)C(C)(C)CCC4(C)C)c3ccc4c(c3)C(C)(C)CCC4(C)C)c2)c(-c2ccccc2)c1 ; CC(C)(C)c1ccc(N2c3cc(Cl)ccc3B3c4cc5c(cc4N(c4ccc6c(c4)C(C)(C)CCC6(C)C)c4cc(Cl)cc2c43)C(C)(C)CCC5(C)C)c(-c2ccccc2)c1]

After Intermediate 42 (25 g) and 20.0 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Intermediate 43 (7.4 g) (yield 29%). MS[M+H]+=843

### 4) Synthesis of Compound 25

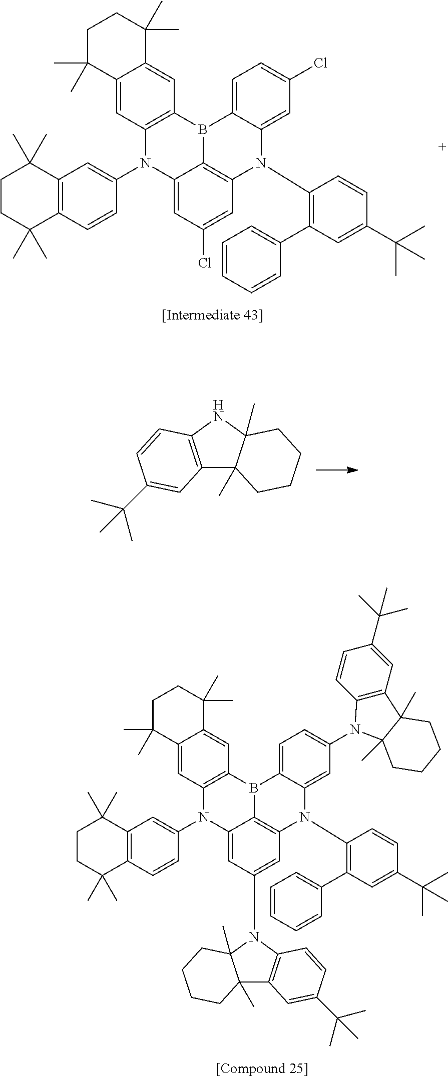

After Intermediate 43 (7 g), 4.3 g of 6-(tert butyl)-4a,9a-dimethyl-2,3,4,4a,9,9a-hexahydro-1H-carbazole, 1.6 g of sodium-tert-butoxide, and 0.05 g of bis(tri-tert-butylphosphine)palladium(0) were put into 100 ml of toluene under a nitrogen atmosphere, the resulting mixture was stirred under reflux for 6 hours. After the completion of the reaction, the resulting product was extracted, and then recrystallized to obtain Compound 25 (7.7 g). (Yield 72%). MS[M+H]+=1284

### Synthesis Example 27. Synthesis of Compound 27

### 1) Synthesis of Intermediate 44

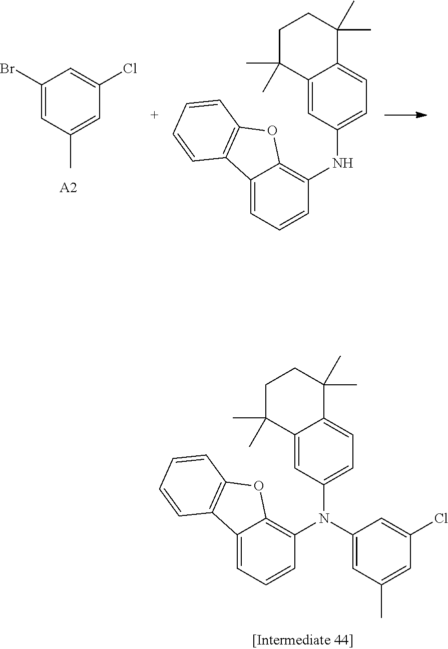

After A2 (40 g), 71.9 g of N-(5,5,8,8-tetramethyl-5,6,7,8-tetrahydronaphthalen-2-yl)dibenzo[b,d]furan-4-amine, 37.4 g of sodium-tert-butoxide, and 1.0 g of bis(tri-tert-butylphosphine)palladium(0) were put into 600 ml of toluene under a nitrogen atmosphere, the resulting mixture was stirred under reflux for 2 hours. After the completion of the reaction, the resulting product was extracted, and then recrystallized to obtain Intermediate 44 (72 g). (Yield 75%). MS[M+H]+=495

### 2) Synthesis of Intermediate 45

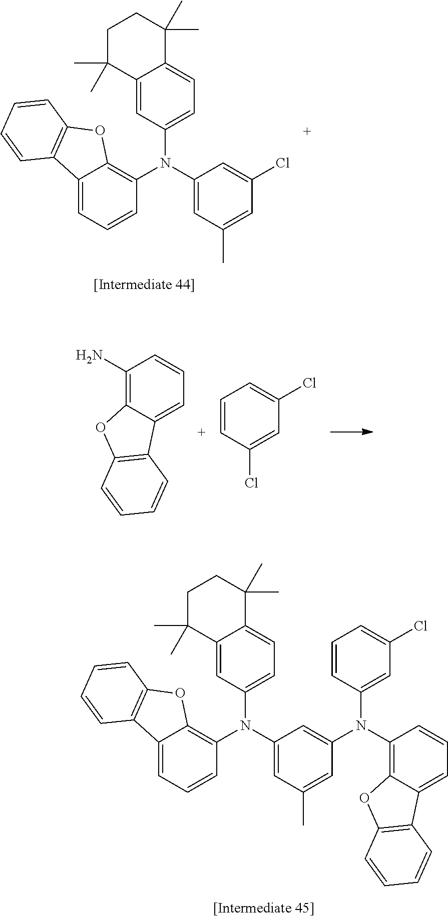

After Intermediate 44 (40 g), 14.8 g of dibenzo[b,d]furan-4-amine, 0.4 g of bis(tri-tert-butylphosphine)palladium(0), and 19 g of sodium-tert-butoxide were put into 600 ml of toluene under a nitrogen atmosphere, the resulting mixture was stirred under reflux for 1 hour. Thereafter, whether the reaction proceeded was confirmed, and then 15.5 g of 1-bromo-3-chlorobenzene was introduced thereinto during the stirring, and then the resulting mixture was stirred under reflux for 4 hours. After the completion of the reaction, the resulting product was extracted, and then recrystallized to obtain Intermediate 45 (45 g). (Yield 74%). MS[M+H]+=752

### 3) Synthesis of Intermediate 46

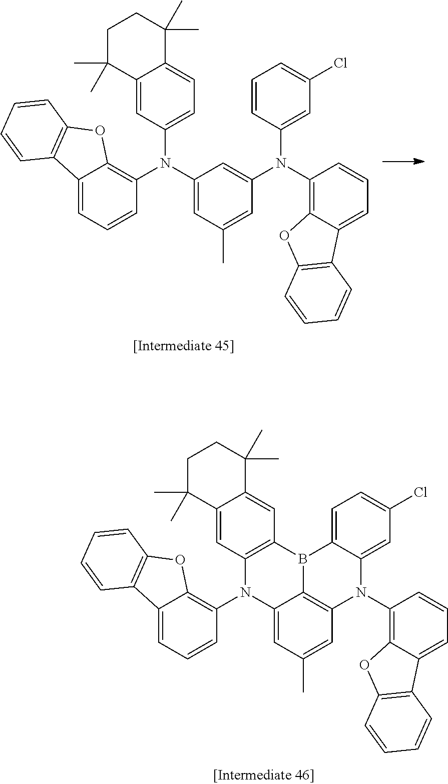 [SMILES: Cc1cc(N(c2cccc(Cl)c2)c2cccc3c2oc2ccccc23)cc(N(c2ccc3c(c2)C(C)(C)CCC3(C)C)c2cccc3c2oc2ccccc23)c1 ; Cc1cc2c3c(c1)N(c1cccc4c1oc1ccccc14)c1cc4c(cc1B3c1ccc(Cl)cc1N2c1cccc2c1oc1ccccc12)C(C)(C)CCC4(C)C]

After Intermediate 45 (25 g) and 22.1 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Intermediate 46 (7.7 g) (yield 30%). MS[M+H]+=760

### 4) Synthesis of Compound 27

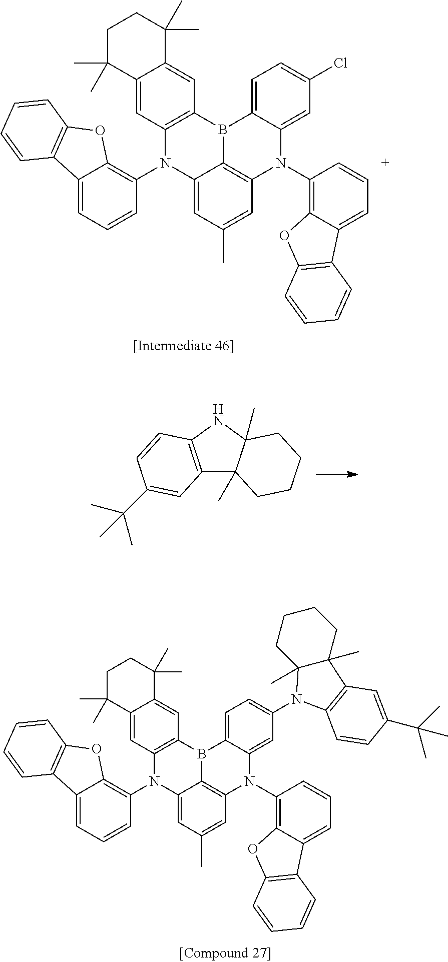 [SMILES: Cc1cc2c3c(c1)N(c1cccc4c1oc1ccccc14)c1cc4c(cc1B3c1ccc(Cl)cc1N2c1cccc2c1oc1ccccc12)C(C)(C)CCC4(C)C ; CC(C)(C)c1ccc2c(c1)C1(C)CCCCC1(C)N2 ; Cc1cc2c3c(c1)N(c1cccc4c1oc1ccccc14)c1cc4c(cc1B3c1ccc(N3c5ccc(C(C)(C)C)cc5C5(C)CCCCC35C)cc1N2c1cccc2c1oc1ccccc12)C(C)(C)CCC4(C)C]

After Intermediate 46 (7 g), 2.4 g of 6-(tert-butyl)-4a,9a-dimethyl-2,3,4,4a,9,9a-hexahydro-1H-carbazole, 1.8 g of sodium-tert-butoxide, and 0.05 g of bis(tri-tert-butylphosphine)palladium(0) were put into 100 ml of toluene under a nitrogen atmosphere, the resulting mixture was stirred under reflux for 6 hours. After the completion of the reaction, the resulting product was extracted, and then recrystallized to obtain Compound 27 (6.7 g). (Yield 74%). MS[M+H]+=981

### Synthesis Example 28. Synthesis of Compound 28

### 1) Synthesis of Intermediate 47

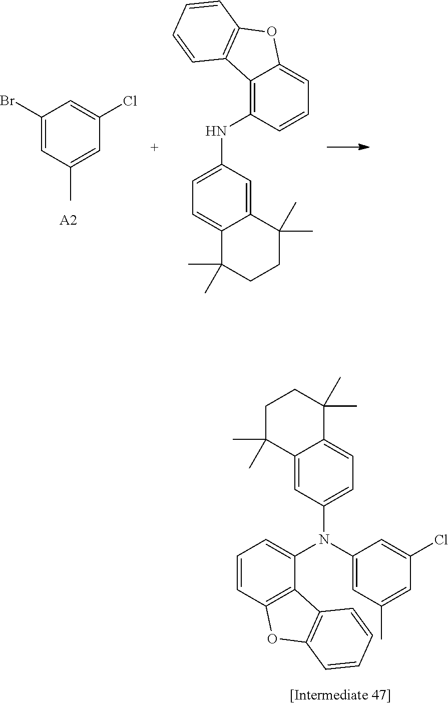

After A2 (40 g), 71.9 g of N-(5,5,8,8-tetramethyl-5,6,7,8-tetrahydronaphthalen-2-yl)dibenzo[b,d]furan-1-amine, 37.4 g of sodium-tert-butoxide, and 1.0 g of bis(tri-tert-butylphosphine)palladium(0) were put into 600 ml of toluene under a nitrogen atmosphere, the resulting mixture was stirred under reflux for 2 hours. After the completion of the reaction, the resulting product was extracted, and then recrystallized to obtain Intermediate 47 (73 g). (Yield 76%). MS[M+H]+=495

### 2) Synthesis of Intermediate 48

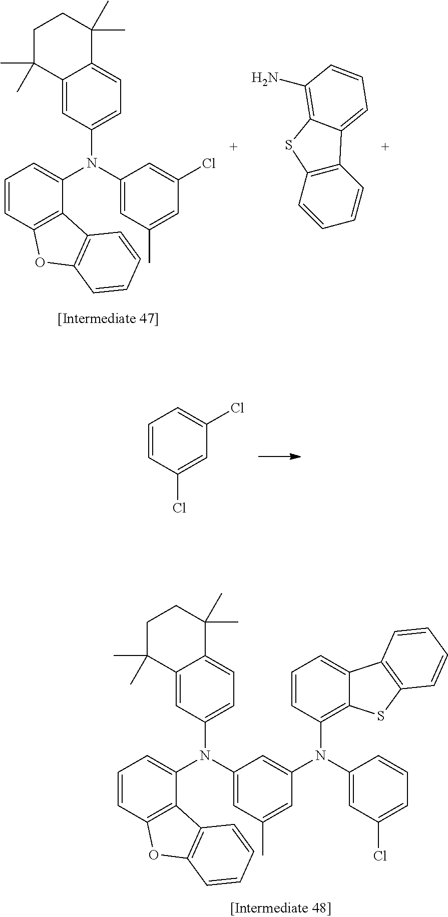

After Intermediate 47 (40 g), 16.1 g of dibenzo[b,d]thiophen-4-amine, and 0.4 g of bis(tri-tert-butylphosphine)palladium(0) were put into 600 ml of toluene under a nitrogen atmosphere, the resulting mixture was stirred under reflux for 1 hour. Thereafter, whether the reaction proceeded was confirmed, and then 15.5 g of 1-bromo-3-chlorobenzene was introduced thereinto during the stirring, and then the resulting mixture was stirred under reflux for 4 hours. After the completion of the reaction, the resulting product was extracted, and then recrystallized to obtain Intermediate 48 (44 g). (Yield 71%). MS[M+H]+=768

### 3) Synthesis of Intermediate 49

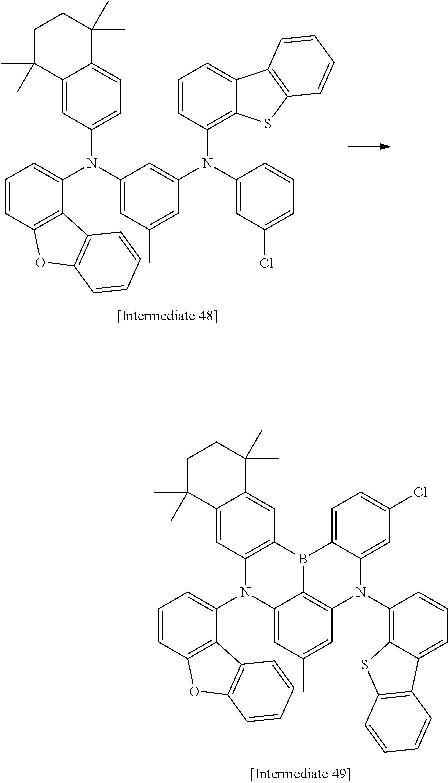

After Intermediate 48 (25 g) and 21.7 g of boron triiodide were put into 250 ml of 1,2-dichlorobenzene under a nitrogen atmosphere, the resulting mixture was stirred at 160° C. for 4 hours. After the completion of the reaction, the resulting product was extracted, and then recrystallized to obtain Intermediate 49 (7.4 g) (yield 29%). MS[M+H]+=776

### 4) Synthesis of Compound 28

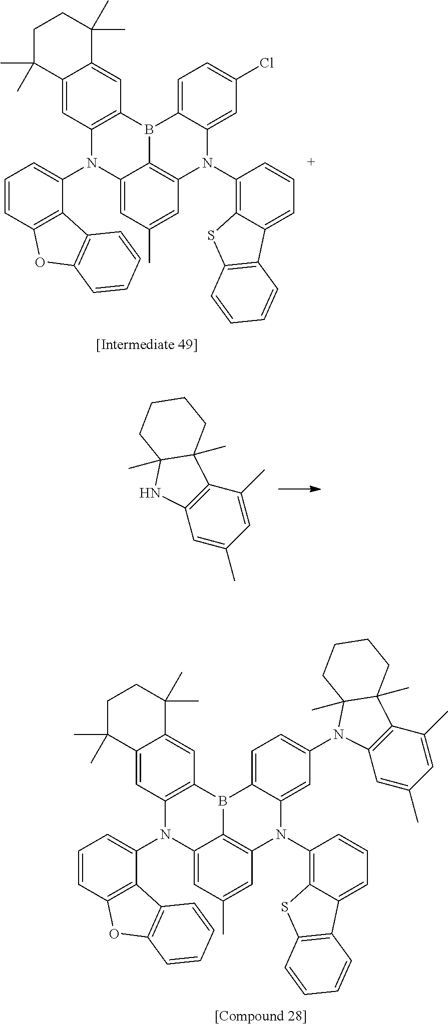

After Intermediate 49 (7 g), 2.1 g of 4a,5,7,9a-tetramethyl-2,3,4,4a,9,9a-hexahydro-1H-carbazole, 1.8 g of sodium-tert-butoxide, and 0.05 g of bis(tri-tert-butylphosphine)palladium(0) were put into 100 ml of toluene under a nitrogen atmosphere, the resulting mixture was stirred under reflux for 6 hours. After the completion of the reaction, the resulting product was extracted, and then recrystallized to obtain Compound 28 (6.8 g). (Yield 78%). MS[M+H]+=969

### Synthesis Example of Chemical Formula 2

### Synthesis Example A-1. Synthesis of Compound A-1

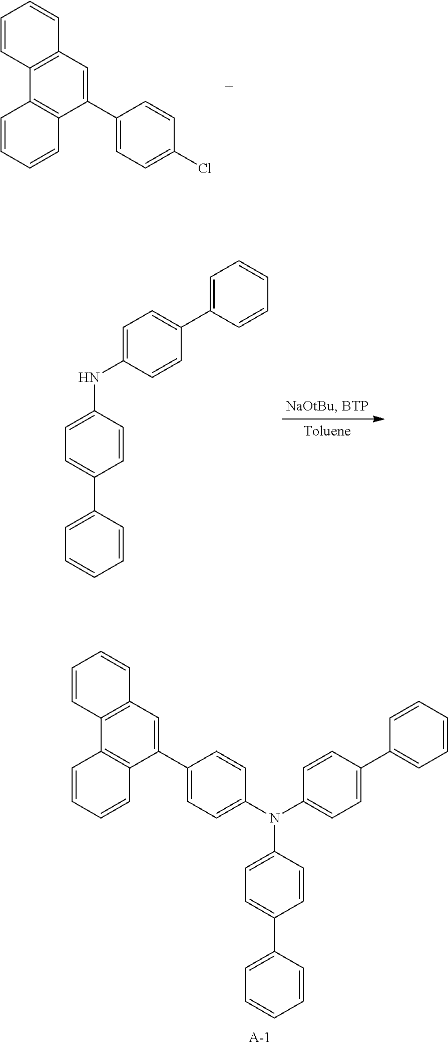

After toluene (150 ml) was added to 9-(4-chlorophenyl)phenanthrene (20.0 g, 69.26 mmol), di([1,1′-biphenyl]-4-yl)amine (22.71 g, 70.64 mmol), and sodium tert-butoxide (NaOtBu) (9.32 g, 96.96 mmol), the resulting mixture was heated and stirred for 10 minutes. After bis(tri-tert-butylphosphine)palladium (BTP) (0.18 g, 0.35 mmol) dissolved in toluene (10 ml) was added to the mixture, the resulting mixture was heated and stirred for 1 hour. After the completion of the reaction and filtration, the layers were separated with toluene and water. After the solvent was removed, the residue was recrystallized with ethyl acetate to obtain Compound A-1 (3102 g, 78.52% yield). (MS[M+H]⁺=574)

### Synthesis Example A-2. Synthesis of Compound A-2

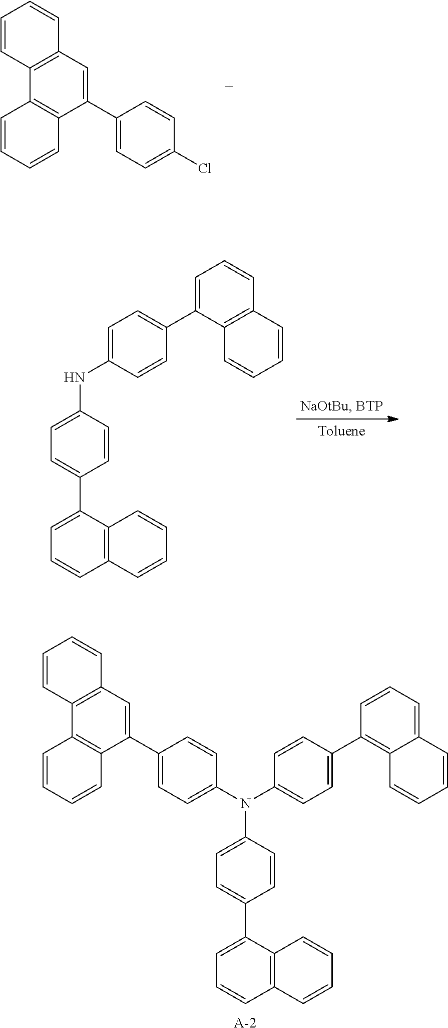

Compound A-2 (37.0 g, 79.28% yield) was obtained using 9-(4-chlorophenyl)phenanthrene (20.0 g, 69.26 mmol) and bis(4-(naphthalen-1-yl)phenyl)amine (29.78 g, 70.64 mmol) in the same manner as in Synthesis Example A-1. (MS[M+H]⁺=674)

### Synthesis Example A-3. Synthesis of Compound A-3

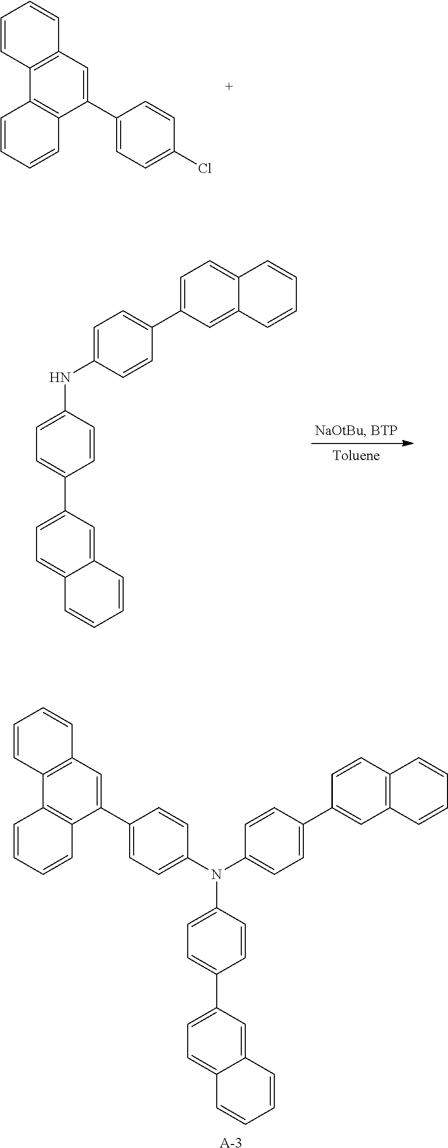

Compound A-3 (36.0 g, 77.13% yield) was obtained using 9-(4-chlorophenyl)phenanthrene (20.0 g, 69.26 mmol) and bis(4-(naphthalen-2-yl)phenyl)amine (29.78 g, 70.64 mmol) in the same manner as in Synthesis Example A-1. (MS[M+H]⁺=674)

### Synthesis Example A-4. Synthesis of Compound A-4

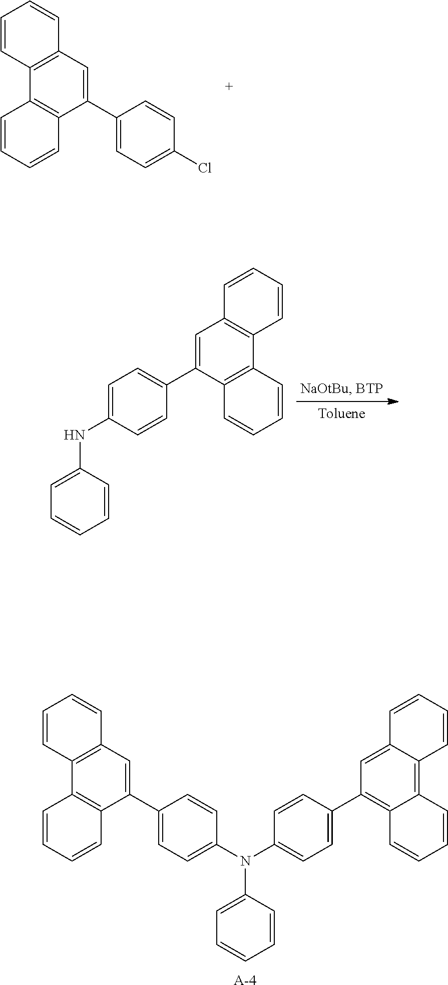

Compound A-4 (33.0 g, 79.71% yield) was obtained using 9-(4-chlorophenyl)phenanthrene (20.0 g, 69.26 mmol) and 4-(phenanthren-9-yl)-N-phenylaniline (24.40 g, 70.64 mmol) in the same manner as in Synthesis Example A-1. (MS[M+H]⁺=598)

### Synthesis Example A-5. Synthesis of Compound A-5

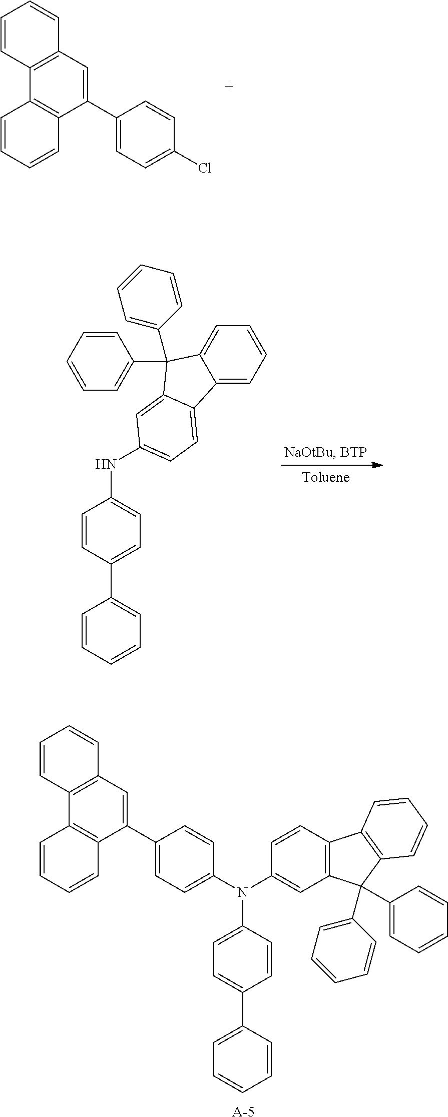

Compound A-5 (40.0 g, 78.26% yield) was obtained using 9-(4-chlorophenyl)phenanthrene (20.0 g, 69.26 mmol) and N-([1,1′-biphenyl]-4-yl)-9,9-diphenyl-9H-fluoren-2-amine (34.30 g, 70.64 mmol) in the same manner as in Synthesis Example A-1. (MS[M+H]⁺=738)

### Synthesis Example A-6. Synthesis of Compound A-6

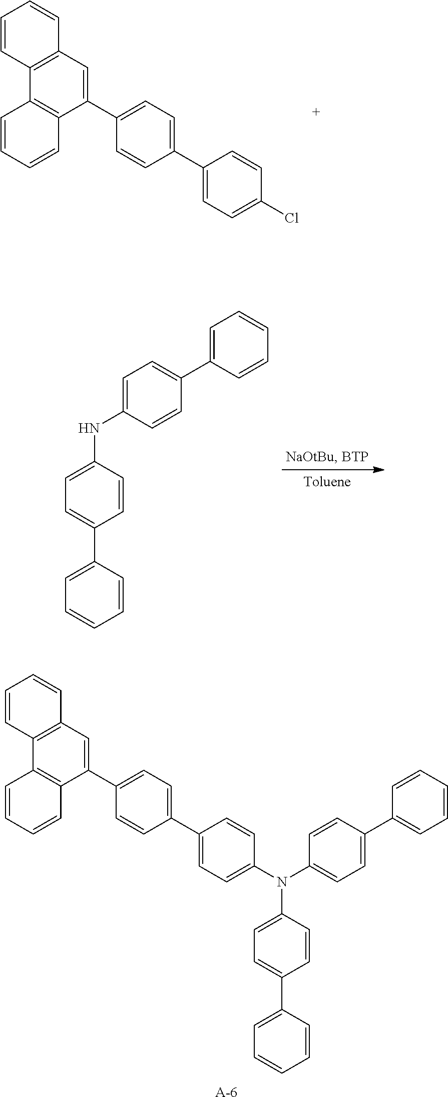 [SMILES: Clc1ccc(-c2ccc(-c3cc4ccccc4c4ccccc34)cc2)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4ccc(-c5cc6ccccc6c6ccccc56)cc4)cc3)cc2)cc1 ; "A-6"]

Compound A-6 (36.0 g, 79.99% yield) was obtained using 9-(4′-chloro-[1,1′-biphenyl]-4-yl)phenanthrene (20.0 g, 69.26 mmol) and di([1,1′-biphenyl]-4-yl)amine (25.77 g, 70.64 mmol) in the same manner as in Synthesis Example A-1. (MS[M+H]+=650)

### EXPERIMENTAL EXAMPLES AND COMPARATIVE EXPERIMENTAL EXAMPLES

### Experimental Example 1-1

A glass substrate thinly coated with indium tin oxide (ITO) to have a thickness of 1,400 Å was put into distilled water in which a detergent was dissolved, and ultrasonically washed. In this case, a product manufactured by the Fischer Co., was used as the detergent, and distilled water twice filtered using a filter manufactured by Millipore Co., was used as the distilled water. After the ITO was washed for 30 minutes, ultrasonic washing was repeated twice by using distilled water for 10 minutes. After the washing using distilled water was completed, ultrasonic washing was conducted by using isopropyl alcohol, acetone, and methanol solvents, and the resulting product was dried and then transported to a plasma washing machine. The substrate was cleaned by using oxygen plasma for 5 minutes, and then was transported to a vacuum deposition machine.

A compound of the following Chemical Formula HAT was thermally vacuum-deposited to have a thickness of 100 Å on the ITO transparent electrode thus prepared, thereby forming a hole injection layer. After a compound of the following Chemical Formula HA was vacuum-deposited to have a thickness of 1150 Å as a first hole transport layer thereon, Compound A-1 prepared in Synthesis Example A-1 was thermally vacuum-deposited to have a thickness of 50 Å as a second hole transport layer. Subsequently, a compound of the following Chemical Formula BH and Compound 1 prepared in Synthesis Example 1 were vacuum-deposited to have a thickness of 200 Å at a weight ratio of 50:1 as a light emitting layer. Subsequently, a compound of the following Chemical Formula HB was vacuum-deposited to have a thickness of 50 Å as a hole blocking layer. Subsequently, a compound of the following Chemical Formula ET and a compound of the following Liq were thermally vacuum-deposited to have a thickness of 310 Å at a weight ratio of 1:1 as a layer which simultaneously transports and injects electrons (an electron injection and transport layer). A cathode was formed by sequentially depositing lithium fluoride (LiF) and aluminum to have a thickness of 12 Å and 1,000 Å, respectively, on the electron injection and transport layer, thereby manufacturing an organic light emitting device.

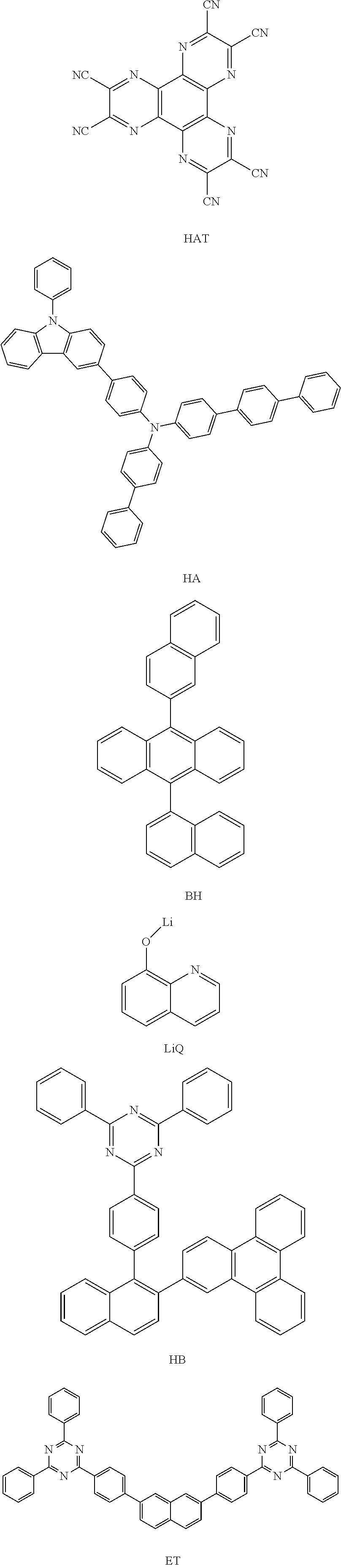

### Experimental Examples 1-2 to 1-74 and Comparative

### Examples 1-1 to 1-36

Organic light emitting devices of Experimental Examples 1-2 to 1-74 and Comparative Examples 1-1 to 1-36 were manufactured in the same manner as in Experimental Example 1-1, except that in Experimental Example 1-1, compounds described in the following Table 1 were used respectively as the light emitting layer instead of Compound 1, and compounds described in the following Table 1 were used respectively as the hole transport layer instead of Compound A-1. When a current of 10 mA/cm² was applied to each of the organic light emitting devices manufactured in the Experimental Examples and the Comparative Examples, the voltage, efficiency, color coordinate, and service life were measured, and the results thereof are shown in the following Table 1. Meanwhile, T95 means the time taken for the luminance to be reduced to 95% of the initial luminance (6000 nit).

TABLE 1

| | Light | | | | COLOR | |
|---|---|---|---|---|---|---|
| | emitting | HOLE | | | COOR- | SERVICE |
| | layer | TRANSPORT | Voltage | Efficiency | DINATE | LIFE |
| | (dopant) | LAYER | (V) | (cd/A) | (x, y) | (T95, hr) |
| EXPERI- | COMPOUND | COMPOUND | 3.65 | 5.99 | 0.141, 0.044 | 200 |
| MENTAL | 1 | A-1 | | | | |
| EXAMPLE | | | | | | |
| 1-1 | | | | | | |
| EXPERI- | COMPOUND | COMPOUND | 3.64 | 5.97 | 0.140, 0.044 | 200 |
| MENTAL | 3 | A-1 | | | | |
| EXAMPLE | | | | | | |
| 1-2 | | | | | | |
| EXPERI- | COMPOUND | COMPOUND | 3.66 | 6.00 | 0.140, 0.044 | 195 |
| MENTAL | 5 | A-1 | | | | |
| EXAMPLE | | | | | | |
| 1-3 | | | | | | |
| EXPERI- | COMPOUND | COMPOUND | 3.67 | 5.97 | 0.140, 0.043 | 200 |
| MENTAL | 6 | A-1 | | | | |
| EXAMPLE | | | | | | |
| 1-4 | | | | | | |
| EXPERI- | COMPOUND | COMPOUND | 3.67 | 6.01 | 0.140, 0.043 | 205 |
| MENTAL | 7 | A-1 | | | | |
| EXAMPLE | | | | | | |
| 1-5 | | | | | | |
| EXPERI- | COMPOUND | COMPOUND | 3.66 | 5.98 | 0.140, 0.044 | 195 |
| MENTAL | 11 | A-1 | | | | |
| EXAMPLE | | | | | | |
| 1-6 | | | | | | |
| EXPERI- | COMPOUND | COMPOUND | 3.64 | 6.02 | 0.140, 0.043 | 200 |
| MENTAL | 15 | A-1 | | | | |
| EXAMPLE | | | | | | |
| 1-7 | | | | | | |
| EXPERI- | COMPOUND | COMPOUND | 3.64 | 5.98 | 0.141, 0.044 | 200 |
| MENTAL | 16 | A-1 | | | | |
| EXAMPLE | | | | | | |
| 1-8 | | | | | | |
| EXPERI- | COMPOUND | COMPOUND | 3.67 | 6.00 | 0.140, 0.043 | 195 |
| MENTAL | 17 | A-1 | | | | |
| EXAMPLE | | | | | | |
| 1-9 | | | | | | |
| EXPERI- | COMPOUND | COMPOUND | 3.68 | 5.96 | 0.140, 0.043 | 205 |
| MENTAL | 21 | A-1 | | | | |
| EXAMPLE | | | | | | |
| 1-10 | | | | | | |
| EXPERI- | COMPOUND | COMPOUND | 3.68 | 5.97 | 0.141, 0.044 | 205 |
| MENTAL | 23 | A-1 | | | | |
| EXAMPLE | | | | | | |
| 1-11 | | | | | | |
| EXPERI- | COMPOUND | COMPOUND | 3.66 | 6.01 | 0.140, 0.043 | 200 |
| NTAL | 24 | A-1 | | | | |
| EXAMPLE | | | | | | |
| 1-12 | | | | | | |
| EXPERI- | COMPOUND | COMPOUND | 3.67 | 6.00 | 0.140, 0.043 | 205 |
| MENTAL | 27 | A-1 | | | | |
| EXAMPLE | | | | | | |
| 1-13 | | | | | | |
| EXPERI- | COMPOUND | COMPOUND | 3.58 | 6.02 | 0.140, 0.043 | 200 |
| MENTAL | 2 | A-2 | | | | |
| EXAMPLE | | | | | | |
| 1-14 | | | | | | |
| EXPERI- | COMPOUND | COMPOUND | 3.59 | 5.99 | 0.141, 0.044 | 200 |
| MENTAL | 3 | A-2 | | | | |
| EXAMPLE | | | | | | |
| 1-15 | | | | | | |
| EXPERI- | COMPOUND | COMPOUND | 3.58 | 6.03 | 0.140, 0.043 | 210 |
| MENTAL | 5 | A-2 | | | | |
| EXAMPLE | | | | | | |
| 1-16 | | | | | | |
| EXPERI- | COMPOUND | COMPOUND | 3.59 | 6.02 | 0.140, 0.043 | 200 |
| MENTAL | 6 | A-2 | | | | |
| EXAMPLE | | | | | | |
| 1-17 | | | | | | |
| EXPERI- | COMPOUND | COMPOUND | 3.56 | 6.04 | 0.140, 0.043 | 205 |
| MENTAL | 8 | A-2 | | | | |
| EXAMPLE | | | | | | |
| 1-18 | | | | | | |
| EXPERI- | COMPOUND | COMPOUND | 3.56 | 6.04 | 0.140, 0.043 | 210 |
| MENTAL | 10 | A-2 | | | | |
| EXAMPLE | | | | | | |
| 1-19 | | | | | | |
| EXPERI- | COMPOUND | COMPOUND | 3.57 | 6.05 | 0.140, 0.043 | 195 |
| MENTAL | 11 | A-2 | | | | |
| EXAMPLE | | | | | | |
| 1-20 | | | | | | |
| EXPERI- | COMPOUND | COMPOUND | 3.60 | 5.99 | 0.141, 0.043 | 205 |
| MENTAL | 12 | A-2 | | | | |
| EXAMPLE | | | | | | |
| 1-21 | | | | | | |
| EXPERI- | COMPOUND | COMPOUND | 3.58 | 6.02 | 0.140, 0.043 | 205 |
| MENTAL | 14 | A-2 | | | | |
| EXAMPLE | | | | | | |
| 1-22 | | | | | | |
| EXPERI- | COMPOUND | COMPOUND | 3.57 | 6.03 | 0.140, 0.043 | 210 |
| MENTAL | 16 | A-2 | | | | |
| EXAMPLE | | | | | | |
| 1-23 | | | | | | |
| EXPERI- | COMPOUND | COMPOUND | 3.56 | 6.04 | 0.140, 0.043 | 210 |
| MENTAL | 18 | A-2 | | | | |
| EXAMPLE | | | | | | |
| 1-24 | | | | | | |
| EXPERI- | COMPOUND | COMPOUND | 3.57 | 6.05 | 0.140, 0.043 | 205 |
| MENTAL | 20 | A-2 | | | | |
| EXAMPLE | | | | | | |
| 1-25 | | | | | | |
| EXPERI- | COMPOUND | COMPOUND | 3.56 | 6.03 | 0.140, 0.043 | 195 |
| MENTAL | 23 | A-2 | | | | |
| EXAMPLE | | | | | | |
| 1-26 | | | | | | |
| EXPERI- | COMPOUND | COMPOUND | 3.60 | 5.99 | 0.141, 0.044 | 195 |
| MENTAL | 28 | A-2 | | | | |
| EXAMPLE | | | | | | |
| 1-27 | | | | | | |
| EXPERI- | COMPOUND | COMPOUND | 3.58 | 6.02 | 0.140, 0.044 | 205 |
| MENTAL | 3 | A-3 | | | | |
| EXAMPLE | | | | | | |
| 1-28 | | | | | | |
| EXPERI- | COMPOUND | COMPOUND | 3.60 | 5.99 | 0.140, 0.043 | 200 |
| MENTAL | 4 | A-3 | | | | |
| EXAMPLE | | | | | | |
| 1-29 | | | | | | |
| EXPERI- | COMPOUND | COMPOUND | 3.59 | 6.01 | 0.140, 0.043 | 205 |
| MENTAL | 7 | A-3 | | | | |
| EXAMPLE | | | | | | |
| 1-30 | | | | | | |
| EXPERI- | COMPOUND | COMPOUND | 3.62 | 6.02 | 0.140, 0.043 | 200 |
| MENTAL | 9 | A-3 | | | | |
| EXAMPLE | | | | | | |
| 1-31 | | | | | | |
| EXPERI- | COMPOUND | COMPOUND | 3.61 | 6.00 | 0.140, 0.044 | 195 |
| MENTAL | 12 | A-3 | | | | |
| EXAMPLE | | | | | | |
| 1-32 | | | | | | |
| EXPERI- | COMPOUND | COMPOUND | 3.60 | 5.99 | 0.140, 0.043 | 205 |
| MENTAL | 13 | A-3 | | | | |
| EXAMPLE | | | | | | |
| 1-33 | | | | | | |
| EXPERI- | COMPOUND | COMPOUND | 3.61 | 6.00 | 0.141, 0.043 | 205 |
| MENTAL | 19 | A-3 | | | | |
| EXAMPLE | | | | | | |
| 1-34 | | | | | | |
| EXPERI- | COMPOUND | COMPOUND | 3.61 | 5.98 | 0.141, 0.044 | 200 |
| MENTAL | 22 | A-3 | | | | |
| EXAMPLE | | | | | | |
| 1-35 | | | | | | |
| EXPERI- | COMPOUND | COMPOUND | 3.60 | 6.00 | 0.140, 0.043 | 205 |
| MENTAL | 23 | A-3 | | | | |
| EXAMPLE | | | | | | |
| 1-36 | | | | | | |
| EXPERI- | COMPOUND | COMPOUND | 3.61 | 6.02 | 0.140, 0.044 | 205 |
| MENTAL | 25 | A-3 | | | | |
| EXAMPLE | | | | | | |
| 1-37 | | | | | | |
| EXPERI- | COMPOUND | COMPOUND | 3.59 | 5.98 | 0.140, 0.043 | 195 |
| MENTAL | 26 | A-3 | | | | |
| EXAMPLE | | | | | | |
| 1-38 | | | | | | |
| EXPERI- | COMPOUND | COMPOUND | 3.62 | 6.03 | 0.141, 0.043 | 205 |
| MENTAL | 1 | A-4 | | | | |
| EXAMPLE | | | | | | |
| 1-39 | | | | | | |
| EXPERI- | COMPOUND | COMPOUND | 3.59 | 6.06 | 0.140, 0.043 | 205 |
| MENTAL | 2 | A-4 | | | | |
| EXAMPLE | | | | | | |
| 1-40 | | | | | | |
| EXPERI- | COMPOUND | COMPOUND | 3.59 | 6.06 | 0.140, 0.043 | 205 |
| MENTAL | 5 | A-4 | | | | |
| EXAMPLE | | | | | | |
| 1-41 | | | | | | |
| EXPERI- | COMPOUND | COMPOUND | 3.58 | 6.06 | 0.140, 0.043 | 200 |
| MENTAL | 6 | A-4 | | | | |
| EXAMPLE | | | | | | |
| 1-42 | | | | | | |
| EXPERI- | COMPOUND | COMPOUND | 3.58 | 6.07 | 0.140, 0.043 | 205 |
| MENTAL | 8 | A-4 | | | | |
| EXAMPLE | | | | | | |
| 1-43 | | | | | | |
| EXPERI- | COMPOUND | COMPOUND | 3.58 | 6.06 | 0.140, 0.043 | 200 |
| MENTAL | 10 | A-4 | | | | |
| EXAMPLE | | | | | | |
| 1-44 | | | | | | |
| EXPERI- | COMPOUND | COMPOUND | 3.57 | 6.08 | 0.140, 0.043 | 195 |
| MENTAL | 11 | A-4 | | | | |
| EXAMPLE | | | | | | |
| 1-45 | | | | | | |
| EXPERI- | COMPOUND | COMPOUND | 3.59 | 6.07 | 0.140, 0.043 | 195 |
| MENTAL | 14 | A-4 | | | | |
| EXAMPLE | | | | | | |
| 1-46 | | | | | | |
| EXPERI- | COMPOUND | COMPOUND | 3.58 | 6.08 | 0.140, 0.043 | 200 |
| MENTAL | 16 | A-4 | | | | |
| EXAMPLE | | | | | | |
| 1-47 | | | | | | |
| EXPERI- | COMPOUND | COMPOUND | 3.60 | 6.02 | 0.141, 0.043 | 200 |
| MENTAL | 18 | A-4 | | | | |
| EXAMPLE | | | | | | |
| 1-48 | | | | | | |
| EXPERI- | COMPOUND | COMPOUND | 3.57 | 6.08 | 0.140, 0.043 | 205 |
| MENTAL | 20 | A-4 | | | | |
| EXAMPLE | | | | | | |
| 1-49 | | | | | | |
| EXPERI- | COMPOUND | COMPOUND | 3.58 | 6.08 | 0.140, 0.043 | 210 |
| MENTAL | 23 | A-4 | | | | |
| EXAMPLE | | | | | | |
| 1-50 | | | | | | |
| EXPERI- | COMPOUND | COMPOUND | 3.61 | 6.06 | 0.140, 0.043 | 200 |
| MENTAL | 24 | A-4 | | | | |
| EXAMPLE | | | | | | |
| 1-51 | | | | | | |
| EXPERI- | COMPOUND | COMPOUND | 3.62 | 6.05 | 0.140, 0.044 | 200 |
| MENTAL | 27 | A-4 | | | | |
| EXAMPLE | | | | | | |
| 1-52 | | | | | | |
| EXPERI- | COMPOUND | COMPOUND | 3.60 | 6.06 | 0.140, 0.043 | 200 |
| MENTAL | 3 | A-5 | | | | |
| EXAMPLE | | | | | | |
| 1-53 | | | | | | |
| EXPERI- | COMPOUND | COMPOUND | 3.58 | 6.03 | 0.141, 0.044 | 205 |
| MENTAL | 7 | A-5 | | | | |
| EXAMPLE | | | | | | |
| 1-54 | | | | | | |
| EXPERI- | COMPOUND | COMPOUND | 3.59 | 5.96 | 0.140, 0.043 | 195 |
| MENTAL | 9 | A-5 | | | | |
| EXAMPLE | | | | | | |
| 1-55 | | | | | | |
| EXPERI- | COMPOUND | COMPOUND | 3.55 | 6.04 | 0.140, 0.043 | 215 |
| MENTAL | 10 | A-5 | | | | |
| EXAMPLE | | | | | | |
| 1-56 | | | | | | |
| EXPERI- | COMPOUND | COMPOUND | 3.56 | 6.02 | 0.140, 0.043 | 220 |
| MENTAL | 14 | A-5 | | | | |
| EXAMPLE | | | | | | |
| 1-57 | | | | | | |
| EXPERI- | COMPOUND | COMPOUND | 3.60 | 5.99 | 0.141, 0.043 | 200 |
| MENTAL | 17 | A-5 | | | | |
| EXAMPLE | | | | | | |
| 1-58 | | | | | | |
| EXPERI- | COMPOUND | COMPOUND | 3.59 | 5.99 | 0.141, 0.043 | 205 |
| MENTAL | 19 | A-5 | | | | |
| EXAMPLE | | | | | | |
| 1-59 | | | | | | |
| EXPERI- | COMPOUND | COMPOUND | 3.59 | 6.03 | 0.140, 0.043 | 215 |
| MENTAL | 25 | A-5 | | | | |
| EXAMPLE | | | | | | |
| 1-60 | | | | | | |
| EXPERI- | COMPOUND | COMPOUND | 3.58 | 6.01 | 0.140, 0.043 | 200 |
| MENTAL | 26 | A-5 | | | | |
| EXAMPLE | | | | | | |
| 1-61 | | | | | | |
| EXPERI- | COMPOUND | COMPOUND | 3.59 | 6.03 | 0.140, 0.043 | 205 |
| MENTAL | 27 | A-5 | | | | |
| EXAMPLE | | | | | | |
| 1-62 | | | | | | |
| EXPERI- | COMPOUND | COMPOUND | 3.60 | 6.00 | 0.140, 0.043 | 200 |
| MENTAL | 1 | A-6 | | | | |
| EXAMPLE | | | | | | |
| 1-63 | | | | | | |
| EXPERI- | COMPOUND | COMPOUND | 3.58 | 6.04 | 0.140, 0.043 | 215 |
| MENTAL | 2 | A-6 | | | | |
| EXAMPLE | | | | | | |
| 1-64 | | | | | | |
| EXPERI- | COMPOUND | COMPOUND | 3.57 | 6.05 | 0.140, 0.043 | 210 |
| MENTAL | 5 | A-6 | | | | |
| EXAMPLE | | | | | | |
| 1-65 | | | | | | |
| EXPERI- | COMPOUND | COMPOUND | 3.55 | 6.05 | 0.140, 0.043 | 210 |
| MENTAL | 6 | A-6 | | | | |
| EXAMPLE | | | | | | |
| 1-66 | | | | | | |
| EXPERI- | COMPOUND | COMPOUND | 3.56 | 6.05 | 0.140, 0.043 | 210 |
| MENTAL | 8 | A-6 | | | | |
| EXAMPLE | | | | | | |
| 1-67 | | | | | | |
| EXPERI- | COMPOUND | COMPOUND | 3.56 | 6.04 | 0.140, 0.043 | 210 |
| MENTAL | 11 | A-6 | | | | |
| EXAMPLE | | | | | | |
| 1-68 | | | | | | |
| EXPERI- | COMPOUND | COMPOUND | 3.57 | 6.04 | 0.140, 0.043 | 200 |
| MENTAL | 16 | A-6 | | | | |
| EXAMPLE | | | | | | |
| 1-69 | | | | | | |
| EXPERI- | COMPOUND | COMPOUND | 3.58 | 6.05 | 0.140, 0.043 | 205 |
| MENTAL | 18 | A-6 | | | | |
| EXAMPLE | | | | | | |
| 1-70 | | | | | | |
| EXPERI- | COMPOUND | COMPOUND | 3.62 | 6.01 | 0.141, 0.044 | 205 |
| MENTAL | 21 | A-6 | | | | |
| EXAMPLE | | | | | | |
| 1-71 | | | | | | |
| EXPERI- | COMPOUND | COMPOUND | 3.57 | 6.05 | 0.140, 0.043 | 210 |
| MENTAL | 23 | A-6 | | | | |
| EXAMPLE | | | | | | |
| 1-72 | | | | | | |
| EXPERI- | COMPOUND | COMPOUND | 3.63 | 6.02 | 0.140, 0.044 | 205 |
| MENTAL | 25 | A-6 | | | | |
| EXAMPLE | | | | | | |
| 1-73 | | | | | | |
| EXPERI- | COMPOUND | COMPOUND | 3.64 | 6.00 | 0.140, 0.043 | 200 |
| MENTAL | 28 | A-6 | | | | |
| EXAMPLE | | | | | | |
| 1-74 | | | | | | |
| COMPAR- | COMPOUND | — | 5.12 | 1.05 | 0.164, 0.067 | 10 |
| ATIVE | 1 | | | | | |
| EXAMPLE | | | | | | |
| 1-1 | | | | | | |
| COMPAR- | COMPOUND | — | 5.24 | 0.98 | 0.157, 0.070 | 25 |
| ATIVE | 6 | | | | | |
| EXAMPLE | | | | | | |
| 1-2 | | | | | | |
| COMPAR- | COMPOUND | HT1 | 3.98 | 4.98 | 0.145, 0.049 | 110 |
| ATIVE | 3 | | | | | |
| EXAMPLE | | | | | | |
| 1-3 | | | | | | |
| COMPAR- | COMPOUND | HT2 | 4.20 | 5.00 | 0.145, 0.049 | 95 |
| ATIVE | 3 | | | | | |
| EXAMPLE | | | | | | |
| 1-4 | | | | | | |
| COMPAR- | COMPOUND | HT3 | 3.95 | 4.90 | 0.144, 0.049 | 110 |
| ATIVE | 3 | | | | | |
| EXAMPLE | | | | | | |
| 1-5 | | | | | | |
| COMPAR- | COMPOUND | HT1 | 3.98 | 5.05 | 0.144, 0.048 | 110 |
| ATIVE | 8 | | | | | |
| EXAMPLE | | | | | | |
| 1-6 | | | | | | |
| COMPAR- | COMPOUND | HT2 | 4.18 | 5.06 | 0.145, 0.049 | 100 |
| ATIVE | 8 | | | | | |
| EXAMPLE | | | | | | |
| 1-7 | | | | | | |
| COMPAR- | COMPOUND | HT3 | 3.96 | 4.96 | 0.144, 0.049 | 110 |
| ATIVE | 8 | | | | | |
| EXAMPLE | | | | | | |
| 1-8 | | | | | | |
| COMPAR- | COMPOUND | HT1 | 3.95 | 4.95 | 0.144, 0.049 | 105 |
| ATIVE | 11 | | | | | |
| EXAMPLE | | | | | | |
| 1-9 | | | | | | |
| COMPAR- | COMPOUND | HT2 | 4.15 | 4.98 | 0.144, 0.049 | 95 |
| ATIVE | 11 | | | | | |
| EXAMPLE | | | | | | |
| 1-10 | | | | | | |
| COMPAR- | COMPOUND | HT3 | 3.96 | 4.90 | 0.145, 0.049 | 110 |
| ATIVE | 11 | | | | | |
| EXAMPLE | | | | | | |
| 1-11 | | | | | | |
| COMPAR- | COMPOUND | HT1 | 3.94 | 5.08 | 0.145, 0.049 | 110 |
| ATIVE | 14 | | | | | |
| EXAMPLE | | | | | | |
| 1-12 | | | | | | |
| COMPAR- | COMPOUND | HT2 | 4.14 | 5.10 | 0.144, 0.048 | 105 |
| ATIVE | 14 | | | | | |
| EXAMPLE | | | | | | |
| 1-13 | | | | | | |
| COMPAR- | COMPOUND | HT3 | 3.94 | 4.99 | 0.145, 0.049 | 110 |
| ATIVE | 14 | | | | | |
| EXAMPLE | | | | | | |
| 1-14 | | | | | | |
| COMPAR- | COMPOUND | HT1 | 4.04 | 4.96 | 0.144, 0.049 | 100 |
| ATIVE | 19 | | | | | |
| EXAMPLE | | | | | | |
| 1-15 | | | | | | |
| COMPAR- | COMPOUND | HT2 | 4.22 | 5.00 | 0.144, 0.049 | 90 |
| ATIVE | 19 | | | | | |
| EXAMPLE | | | | | | |
| 1-16 | | | | | | |
| COMPAR- | COMPOUND | HT3 | 3.99 | 4.91 | 0.144, 0.049 | 105 |
| ATIVE | 19 | | | | | |
| EXAMPLE | | | | | | |
| 1-17 | | | | | | |
| COMPAR- | COMPOUND | HT1 | 3.99 | 5.00 | 0.144, 0.049 | 105 |
| ATIVE | 23 | | | | | |
| EXAMPLE | | | | | | |
| 1-18 | | | | | | |
| COMPAR- | COMPOUND | HT2 | 4.19 | 5.02 | 0.145, 0.048 | 95 |
| ATIVE | 23 | | | | | |
| EXAMPLE | | | | | | |
| 1-19 | | | | | | |
| COMPAR- | COMPOUND | HT3 | 3.94 | 4.94 | 0.144, 0.049 | 110 |
| ATIVE | 23 | | | | | |
| EXAMPLE | | | | | | |
| 1-20 | | | | | | |
| COMPAR- | COMPOUND | HT1 | 3.97 | 4.98 | 0.144, 0.048 | 105 |
| ATIVE | 26 | | | | | |
| EXAMPLE | | | | | | |
| 1-21 | | | | | | |
| COMPAR- | COMPOUND | HT2 | 4.19 | 5.01 | 0.144, 0.048 | 95 |
| ATIVE | 26 | | | | | |
| EXAMPLE | | | | | | |
| 1-22 | | | | | | |
| COMPAR- | COMPOUND | HT3 | 3.95 | 4.92 | 0.145, 0.049 | 110 |
| ATIVE | 26 | | | | | |
| EXAMPLE | | | | | | |
| 1-23 | | | | | | |
| COMPAR- | COMPOUND | HT1 | 3.98 | 5.00 | 0.144, 0.049 | 100 |
| ATIVE | 28 | | | | | |
| EXAMPLE | | | | | | |
| 1-24 | | | | | | |
| COMPAR- | COMPOUND | HT2 | 4.21 | 5.01 | 0.144, 0.049 | 90 |
| ATIVE | 28 | | | | | |
| EXAMPLE | | | | | | |
| 1-25 | | | | | | |
| COMPAR- | COMPOUND | HT3 | 3.96 | 4.92 | 0.145, 0.049 | 105 |
| ATIVE | 28 | | | | | |
| EXAMPLE | | | | | | |
| 1-26 | | | | | | |
| COMPAR- | BD1 | COMPOUND | 4.08 | 5.06 | 0.145, 0.049 | 100 |
| ATIVE | | A-1 | | | | |
| EXAMPLE | | | | | | |
| 1-27 | | | | | | |
| COMPAR- | BD2 | COMPOUND | 3.99 | 5.01 | 0.144, 0.049 | 95 |
| ATIVE | | A-1 | | | | |
| EXAMPLE | | | | | | |
| 1-28 | | | | | | |
| COMPAR- | BD1 | COMPOUND | 4.05 | 5.10 | 0.145, 0.049 | 110 |
| ATIVE | | A-2 | | | | |
| EXAMPLE | | | | | | |
| 1-29 | | | | | | |
| COMPAR- | BD2 | COMPOUND | 3.96 | 5.05 | 0.144, 0.049 | 105 |
| ATIVE | | A-2 | | | | |
| EXAMPLE | | | | | | |
| 1-30 | | | | | | |
| COMPAR- | BD1 | COMPOUND | 4.08 | 5.12 | 0.145, 0.049 | 105 |
| ATIVE | | A-4 | | | | |
| EXAMPLE | | | | | | |
| 1-31 | | | | | | |
| COMPAR- | BD2 | COMPOUND | 4.01 | 5.06 | 0.144, 0.049 | 105 |
| ATIVE | | A-4 | | | | |
| EXAMPLE | | | | | | |
| 1-32 | | | | | | |
| COMPAR- | BD1 | COMPOUND | 4.02 | 5.07 | 0.145, 0.049 | 120 |
| ATIVE | | A-5 | | | | |
| EXAMPLE | | | | | | |
| 1-33 | | | | | | |
| COMPAR- | BD2 | COMPOUND | 3.94 | 5.01 | 0.144, 0.049 | 110 |
| ATIVE | | A-5 | | | | |
| EXAMPLE | | | | | | |
| 1-34 | | | | | | |
| COMPAR- | BD1 | COMPOUND | 4.04 | 5.08 | 0.145, 0.049 | 105 |
| TIVE | | A-6 | | | | |
| EXAMPLE | | | | | | |
| 1-35 | | | | | | |
| COMPAR- | BD2 | COMPOUND | 3.95 | 5.03 | 0.144, 0.049 | 100 |
| ATIVE | | A-6 | | | | |
| EXAMPLE | | | | | | |
| 1-36 | | | | | | |

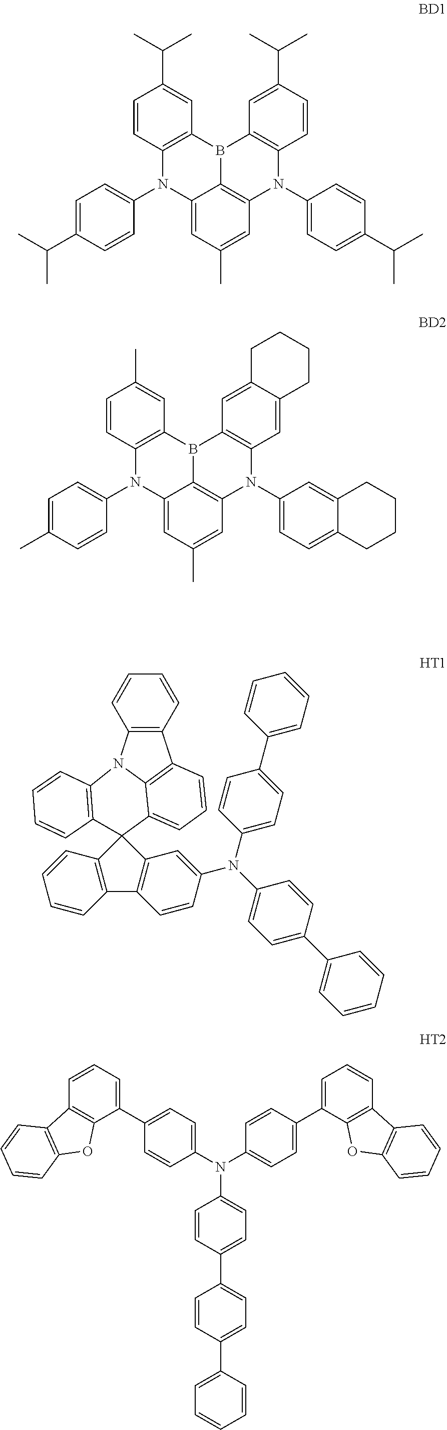

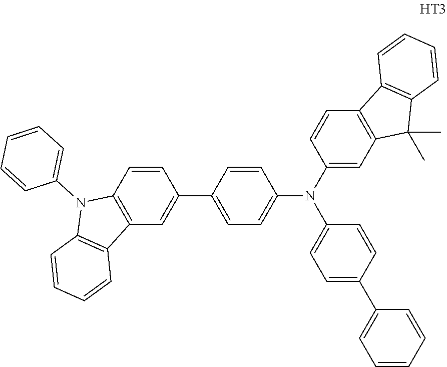 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]

As shown in Table 1, it was confirmed that an organic light emitting device in which the compound of Chemical Formula 1 of the present invention was used as a light emitting layer and the compound of Chemical Formula 2 was used as a hole transport layer exhibited remarkable effects in terms of driving voltage, efficiency, and service life.

1. An organic light emitting device comprising:
an anode;
a cathode provided to face the anode; and
an organic material layer between the anode and the cathode,
wherein the organic material layer comprises a light emitting layer and a first organic material layer provided between the anode and the light emitting layer,
the light emitting layer comprises a compound of the following Chemical Formula 1, and
the first organic material layer comprises a compound of the following Chemical Formula 2: 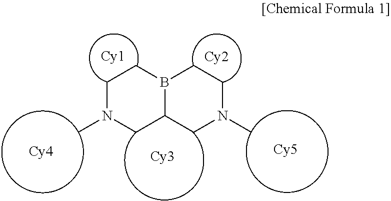
wherein in Chemical Formula 1;
Cy1 to Cy5 are the same as or different from each other, and are each independently one selected from the group consisting of a substituted or unsubstituted aromatic hydrocarbon ring, a substituted or unsubstituted aliphatic hydrocarbon ring, and a substituted or unsubstituted aromatic hetero ring, or a ring in which two or more rings selected from the above group are fused;
one or more of Cy1 to Cy5 are a ring of the following Chemical Formula 1-A; 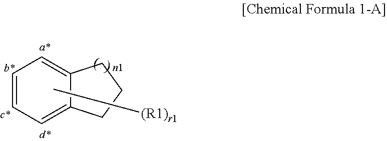
wherein in Chemical Formula 1-A;
one to three of a* to d* are a position that is fused to or linked to Chemical Formula 1;
R1 is hydrogen, deuterium a cyano group, a halogen group, a substituted or unsubstituted alkyl group, a substituted or unsubstituted alkoxy group, a substituted or unsubstituted alkylthio group, a substituted or unsubstituted cycloalkyl group, a substituted or unsubstituted silyl group, a substituted or unsubstituted aryl group, a substituted or unsubstituted aryloxy group, a substituted or unsubstituted arylthio group, a substituted or unsubstituted heterocyclic group, or a substituted or unsubstituted amine group, or is bonded to an adjacent substituent to form a substituted or unsubstituted ring;
n1 is 1 or 2;
r1 is an integer from 0 to 11, and when r1 is 2 or higher, the R1s are the same as or different from each other; 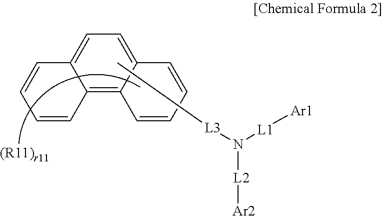
wherein in Chemical Formula 2;
L1 to L3 are the same as or different from each other, and are each independently a direct bond, a substituted or unsubstituted arylene group, or a substituted or unsubstituted divalent heterocyclic group;
Ar1 and Ar2 are the same as or different from each other, and are each independently a substituted or unsubstituted aryl group or a substituted or unsubstituted heterocyclic group;
R11 is hydrogen, deuterium, a cyano group, a halogen group, a substituted or unsubstituted alkyl group, a substituted or unsubstituted alkoxy group, a substituted or unsubstituted alkylthio group, a substituted or unsubstituted cycloalkyl group, a substituted or unsubstituted silyl group, a substituted or unsubstituted aryl group, a substituted or unsubstituted aryloxy group, a substituted or unsubstituted arylthio group, a substituted or unsubstituted heterocyclic group, or a substituted or unsubstituted amine group, or is bonded to an adjacent substituent to form a substituted or unsubstituted ring; and
r11 is an integer from 0 to 9, and when r11 is 2 or higher, the R11s are the same as or different from each other.

2. The organic light emitting device of claim 1, wherein Chemical Formula 1 is the following Chemical Formula 101 or 102: 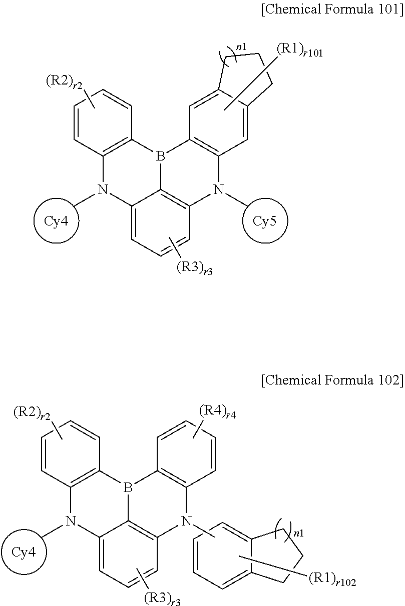
wherein in Chemical Formulae 101 and 102;
Cy4, Cy5, R1, and n1 are the same as those defined in Chemical Formula 1;
R2 to R4 are the same as or different from each other, and are each independently hydrogen, deuterium, a cyano group, a halogen group, a substituted or unsubstituted alkyl group, a substituted or unsubstituted alkoxy group, a substituted or unsubstituted alkylthio group, a substituted or unsubstituted cycloalkyl group, a substituted or unsubstituted silyl group, a substituted or unsubstituted aryl group, a substituted or unsubstituted aryloxy group, a substituted or unsubstituted arylthio group, a substituted or unsubstituted heterocyclic group, or a substituted or unsubstituted amine group, or are bonded to an adjacent substituent to form a substituted or unsubstituted ring;
r101 is an integer from 0 to 10, r102 is an integer from 0 to 11, r2 and r4 are an integer from 0 to 4, and r3 is an integer from 0 to 3; and
when r101, r102, and r2 to r4 are each 2 or higher, substituents in the parenthesis are the same as or different from each other.

3. The organic light emitting device of claim 1, wherein Chemical Formula 1-A is any one of the following Chemical Formulae 1-A-1 to 1-A-3: 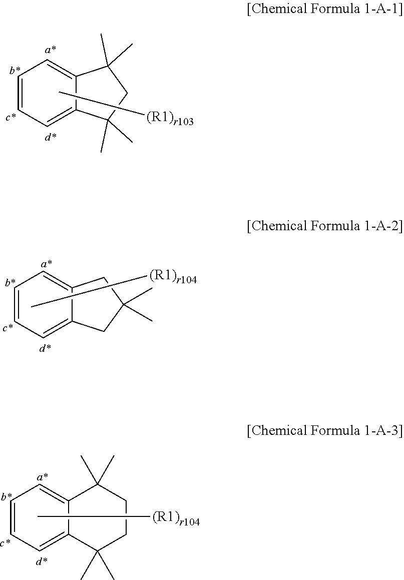
wherein in Chemical Formulae 1-A-1 to 1-A-3;
a* to d* and R1 are the same as those defined in Chemical Formula 1-A;
r103 is an integer from 0 to 5, and r104 is an integer from 0 to 7; and
when r103 and r104 are each 2 or higher, R1's are the same as or different from each other.

4. The organic light emitting device of claim 1, wherein Chemical Formula 1 is any one of the following Chemical Formulae 111 to 118: 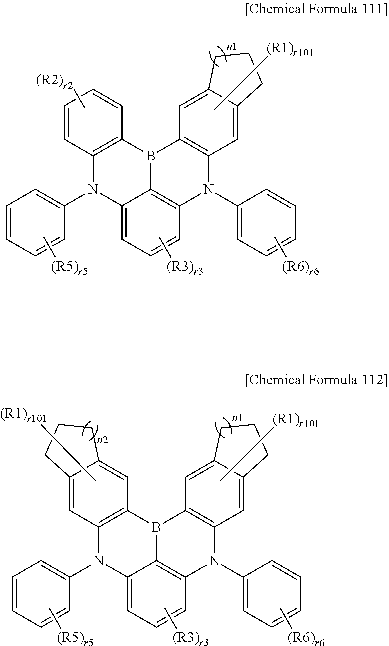 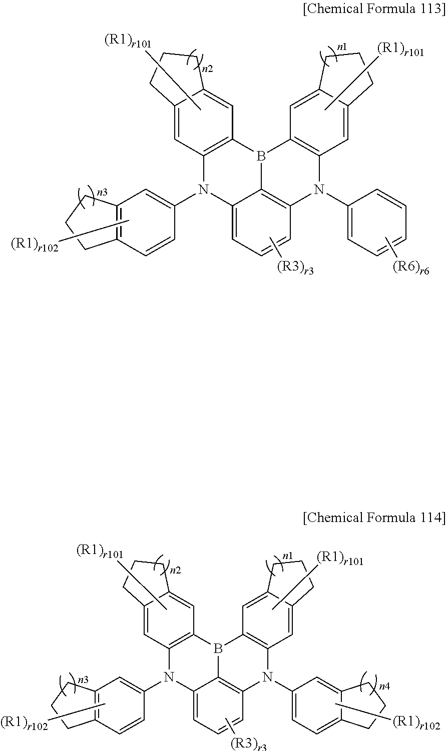 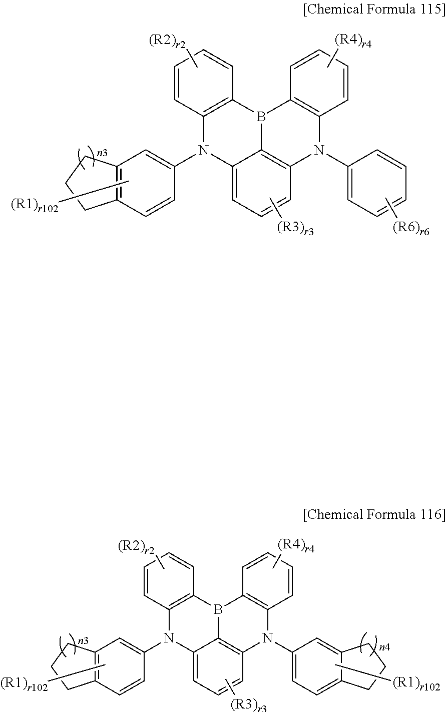 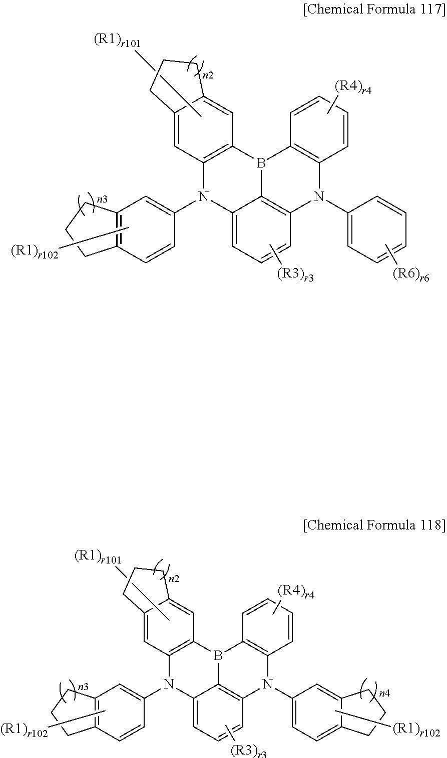
wherein in Chemical Formulae 111 to 118
R1 to R6 are the same as or different from each other, and are each independently hydrogen, deuterium, a cyano group, a halogen group, a substituted or unsubstituted alkyl group, a substituted or unsubstituted alkoxy group, a substituted or unsubstituted alkylthio group, a substituted or unsubstituted cycloalkyl group, a substituted or unsubstituted silyl group, a substituted or unsubstituted aryl group, a substituted or unsubstituted aryloxy group, a substituted or unsubstituted arylthio group, a substituted or unsubstituted heterocyclic group, or a substituted or unsubstituted amine group, or are bonded to an adjacent substituent to form a substituted or unsubstituted ring;
n1 to n4 are each 1 or 2,
r2 and r4 are an integer from 0 to 4, r3 is an integer from 0 to 3, r5 and r6 are an integer from 0 to 5, r101 is an integer from 0 to 10, and r102 is an integer from 0 to 11; and
when r2 to r6, r101, and r102 are each 2 or higher, substituents in the parenthesis are the same as or different from each other.

5. The organic light emitting device of claim 1, wherein Chemical formula is the following Chemical Formula 201: 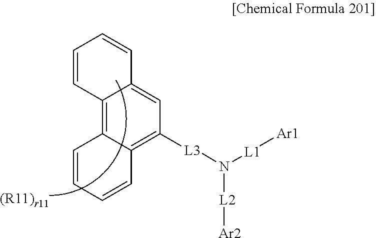
wherein in Chemical Formula 201;
L1 to L3, Ar1, Ar2, R11, and r11 are the same as those defined in Chemical Formula 2.

6. The organic light emitting device of claim 1, wherein Ar1 and Ar2 are each independently selected from among the following structures: 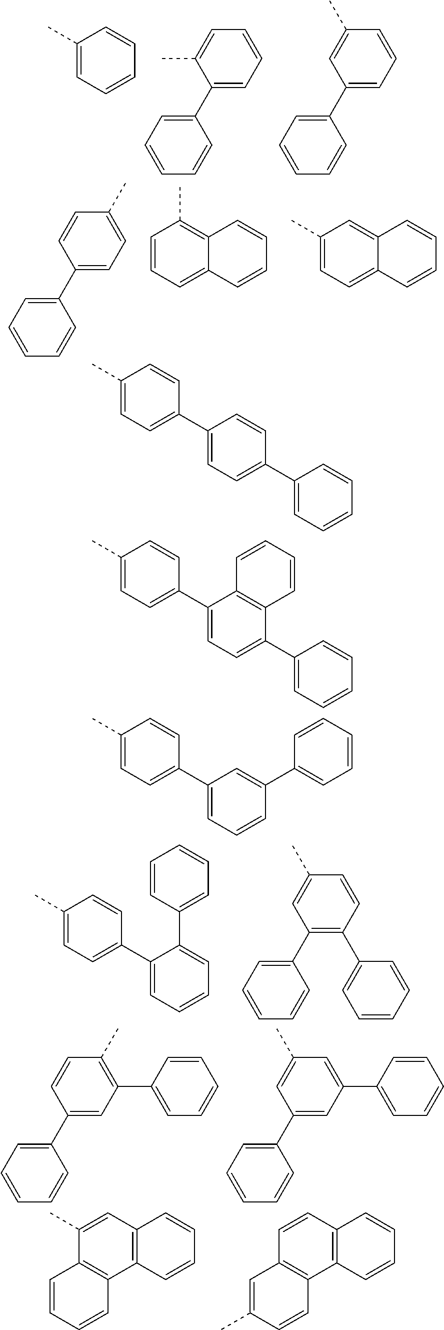 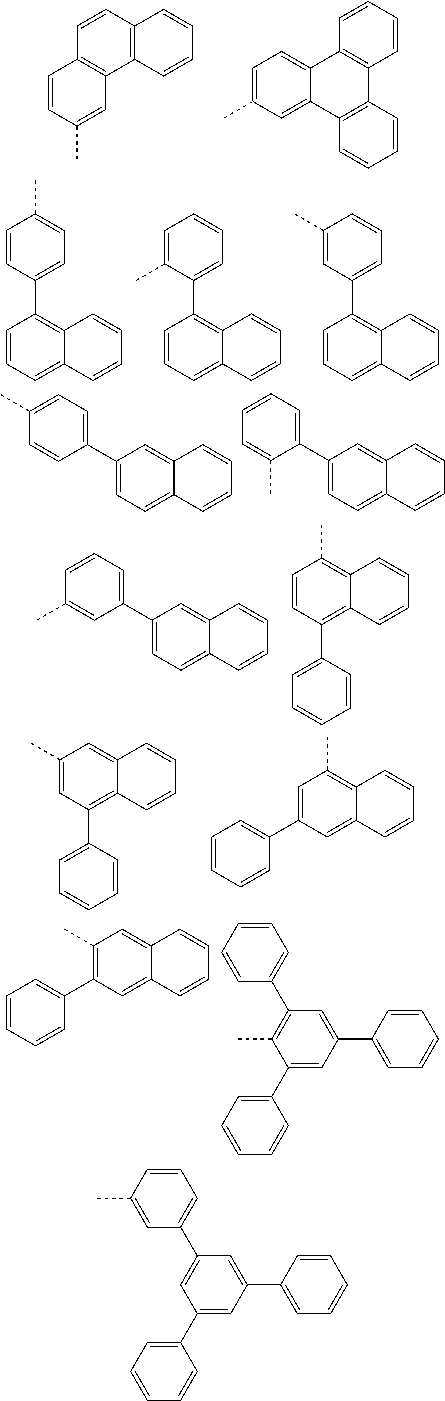 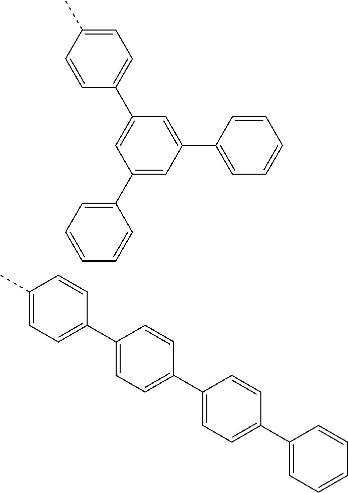 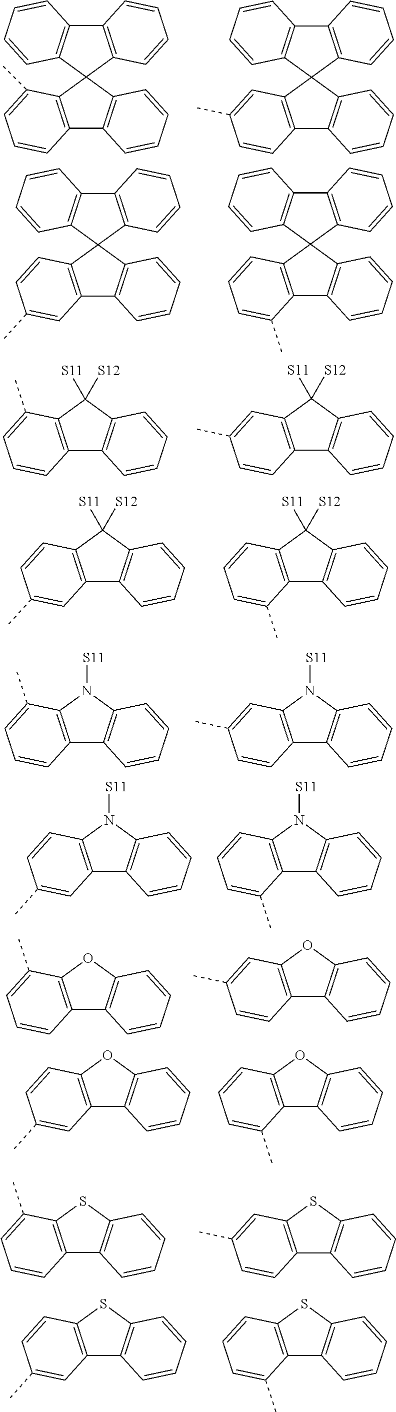
wherein in the structures;
a dotted line denotes a bonding position; and
S11 and S12 are the same as or different from each other, and are each independently hydrogen, a substituted or unsubstituted alkyl group, or a substituted or unsubstituted aryl group.

7. The organic light emitting device of claim 1, wherein L1 to L3 are the same as or different from each other, and are each independently is a direct bond or selected from among the following structures: 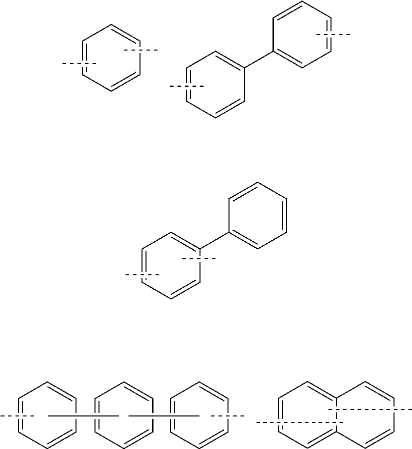
wherein in the structures, the dotted line is a bonding position.

8. The organic light emitting device of claim 1, wherein the compound of Chemical Formula 1 is one compound selected from among the following compounds:

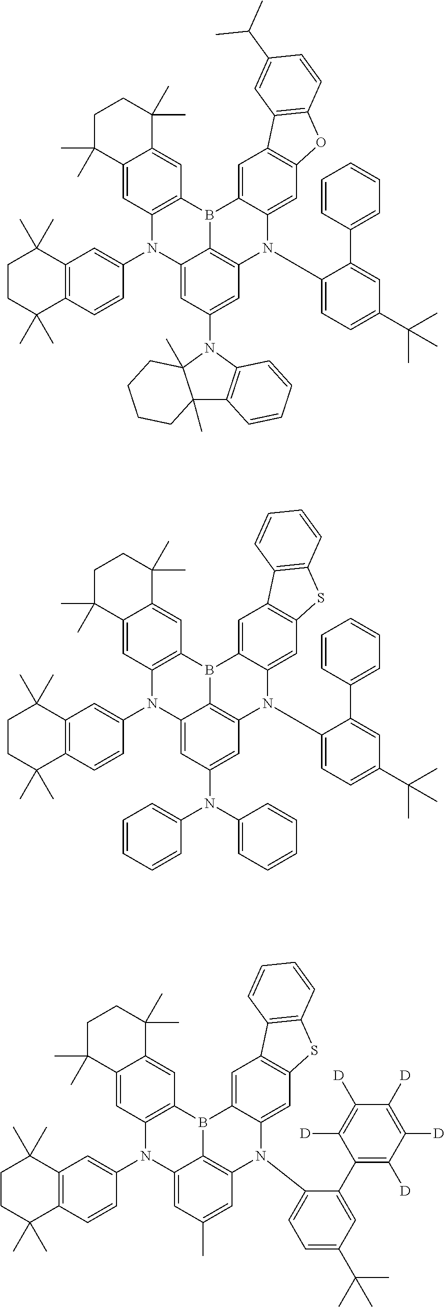 [SMILES: CC(C)c1ccc2oc3cc4c(cc3c2c1)B1c2cc3c(cc2N(c2ccc5c(c2)C(C)(C)CCC5(C)C)c2cc(N5c6ccccc6C6(C)CCCCC56C)cc(c21)N4c1ccc(C(C)(C)C)cc1-c1ccccc1)C(C)(C)CCC3(C)C ; CC(C)(C)c1ccc(N2c3cc4sc5ccccc5c4cc3B3c4cc5c(cc4N(c4ccc6c(c4)C(C)(C)CCC6(C)C)c4cc(N(c6ccccc6)c6ccccc6)cc2c43)C(C)(C)CCC5(C)C)c(-c2ccccc2)c1 ; [2H]c1c([2H])c([2H])c(-c2cc(C(C)(C)C)ccc2N2c3cc4sc5ccccc5c4cc3B3c4cc5c(cc4N(c4ccc6c(c4)C(C)(C)CCC6(C)C)c4cc(C)cc2c43)C(C)(C)CCC5(C)C)c([2H])c1[2H]]

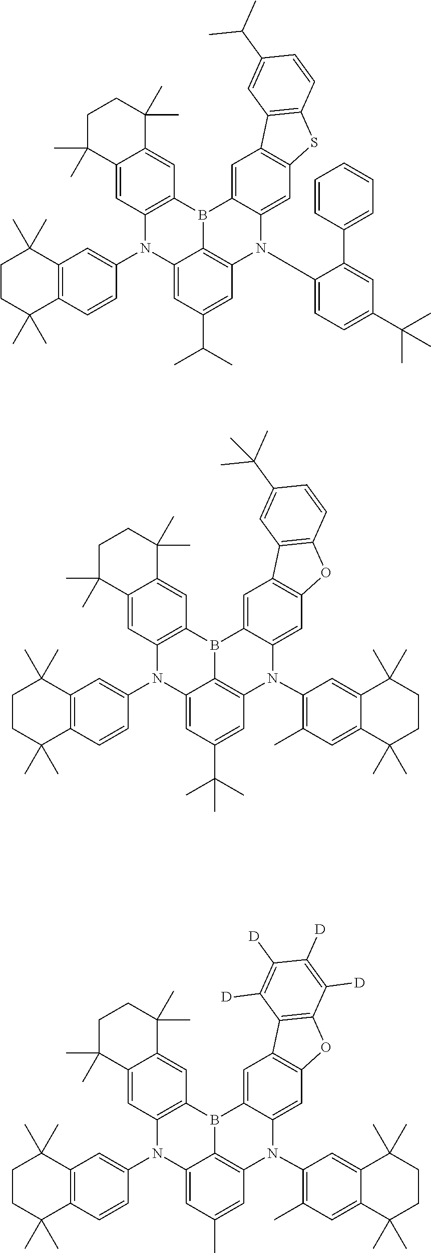 [SMILES: CC(C)c1cc2c3c(c1)N(c1ccc(C(C)(C)C)cc1-c1ccccc1)c1cc4sc5ccc(C(C)C)cc5c4cc1B3c1cc3c(cc1N2c1ccc2c(c1)C(C)(C)CCC2(C)C)C(C)(C)CCC3(C)C ; Cc1cc2c(cc1N1c3cc4oc5ccc(C(C)(C)C)cc5c4cc3B3c4cc5c(cc4N(c4ccc6c(c4)C(C)(C)CCC6(C)C)c4cc(C(C)(C)C)cc1c43)C(C)(C)CCC5(C)C)C(C)(C)CCC2(C)C ; [2H]c1c([2H])c([2H])c2c(oc3cc4c(cc32)B2c3cc5c(cc3N(c3ccc6c(c3)C(C)(C)CCC6(C)C)c3cc(C)cc(c32)N4c2cc3c(cc2C)C(C)(C)CCC3(C)C)C(C)(C)CCC5(C)C)c1[2H]]

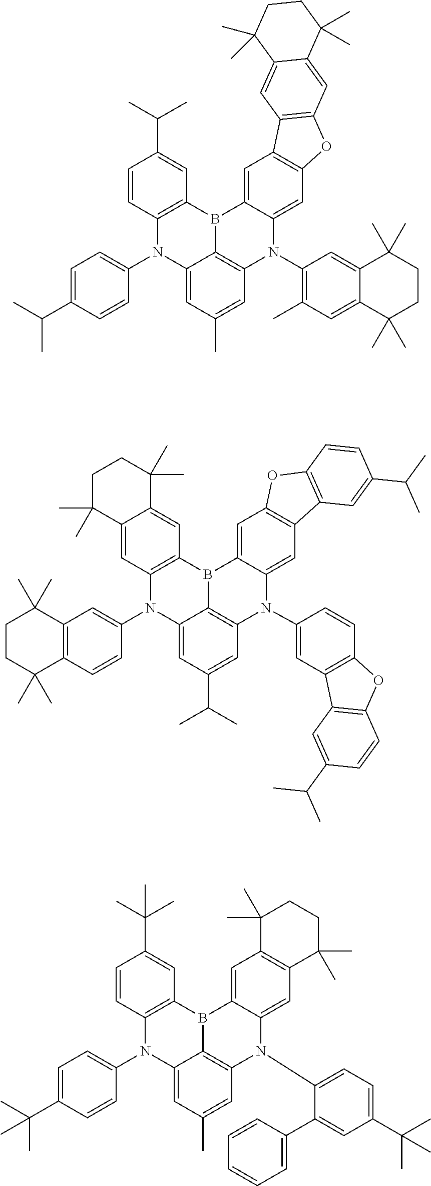

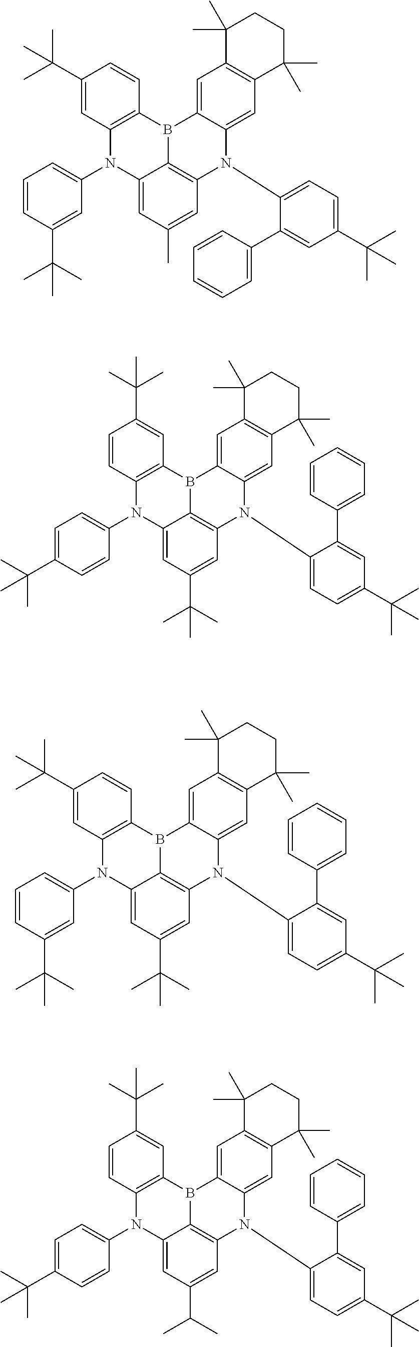

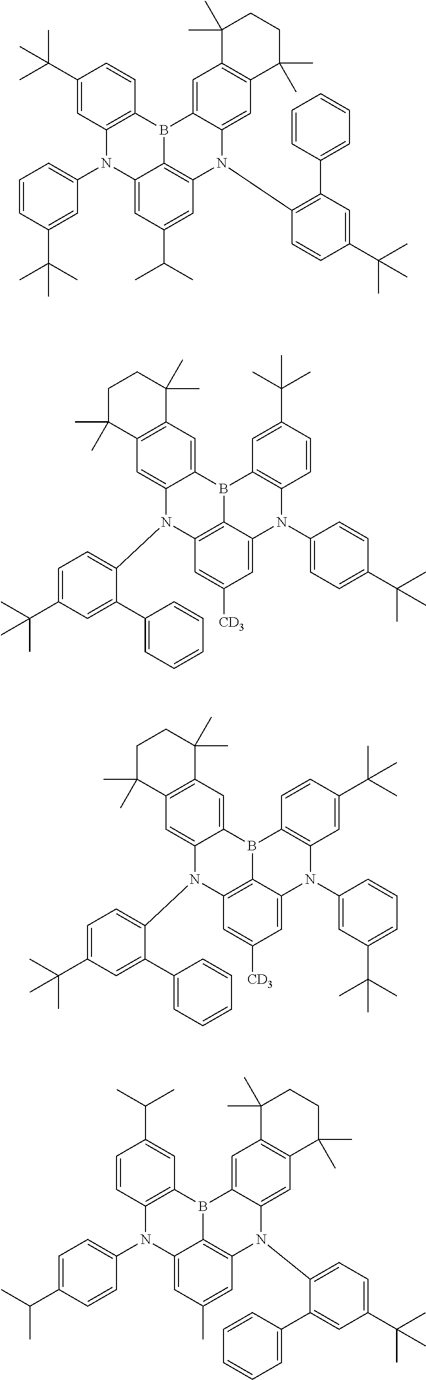

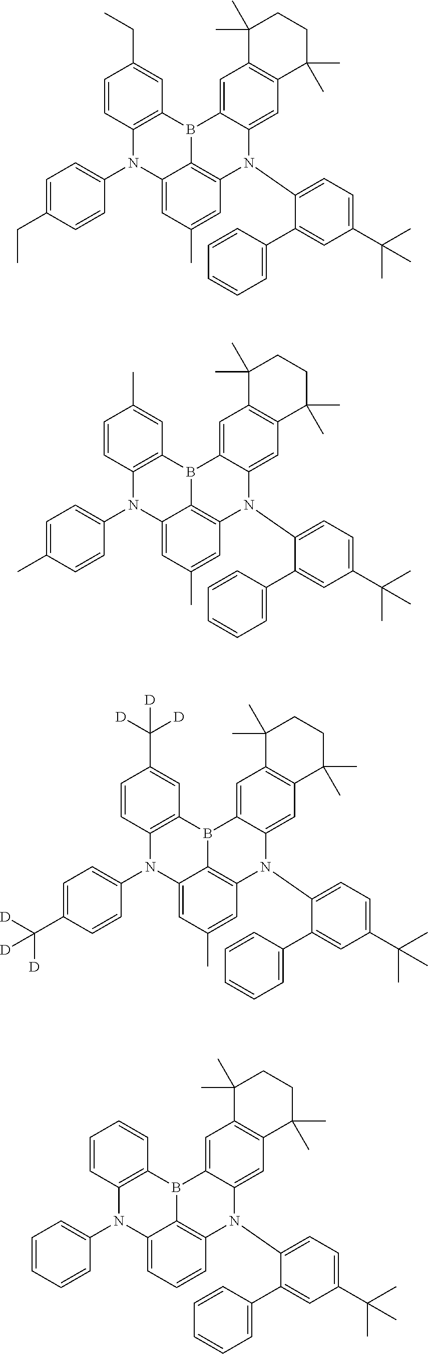

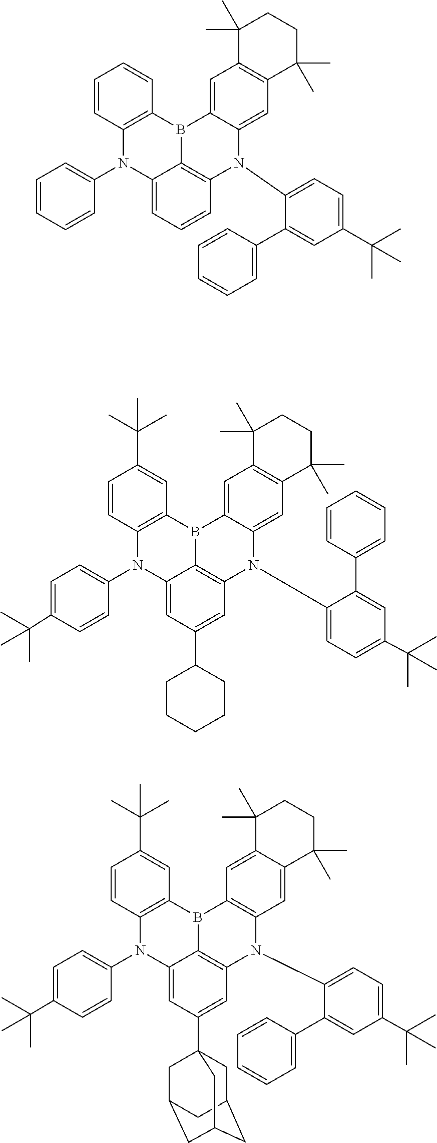

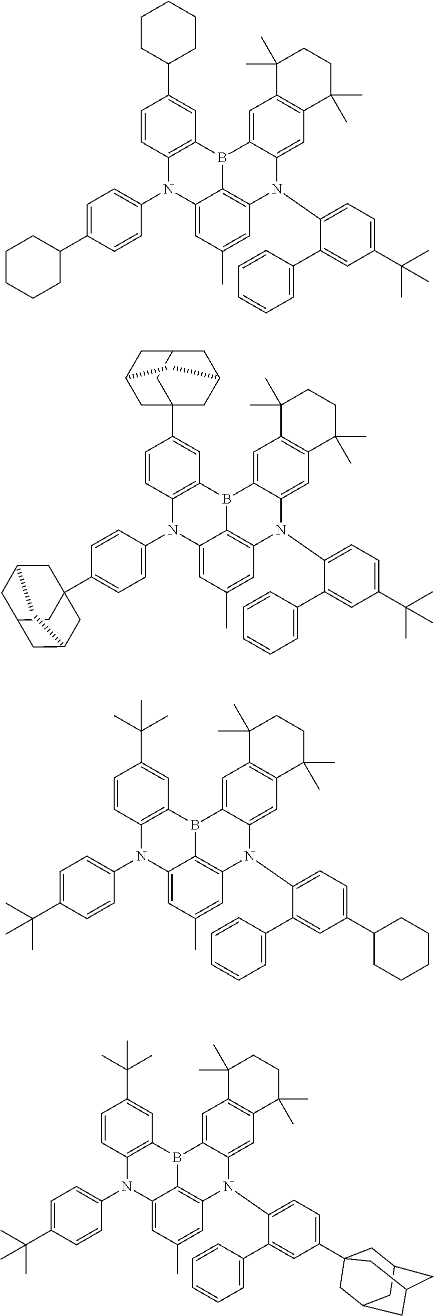

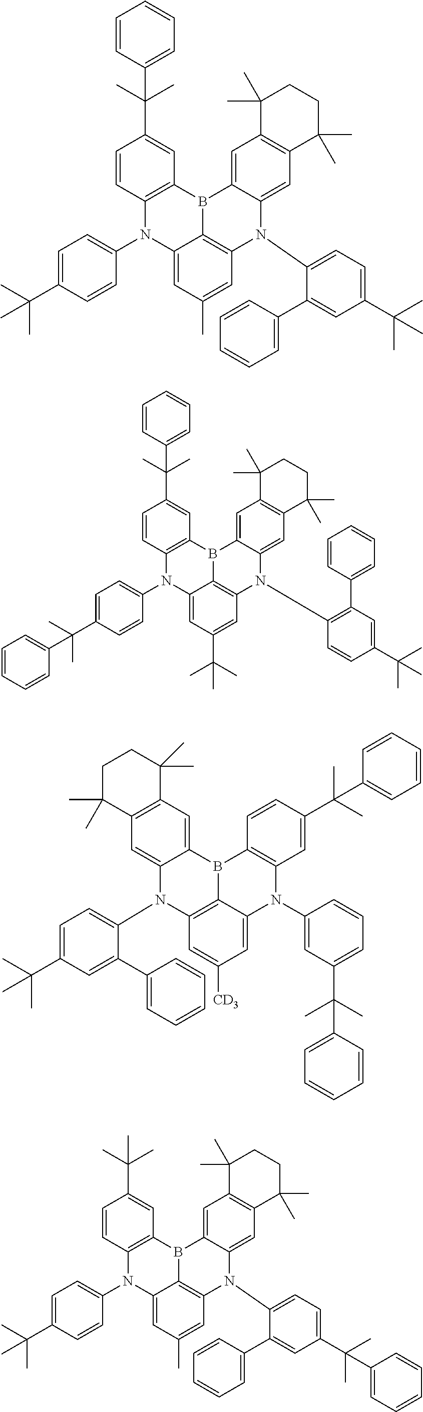

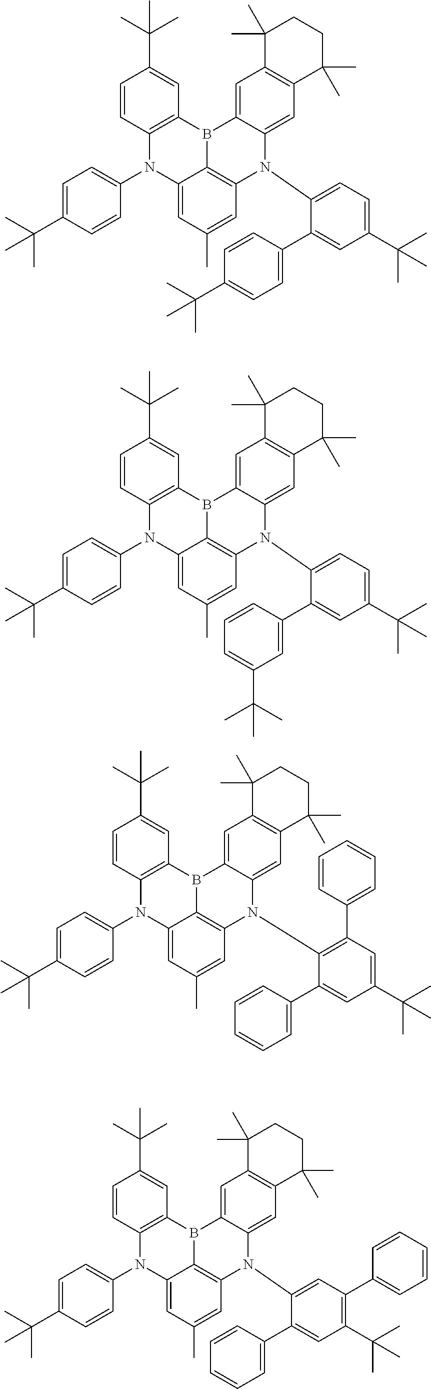

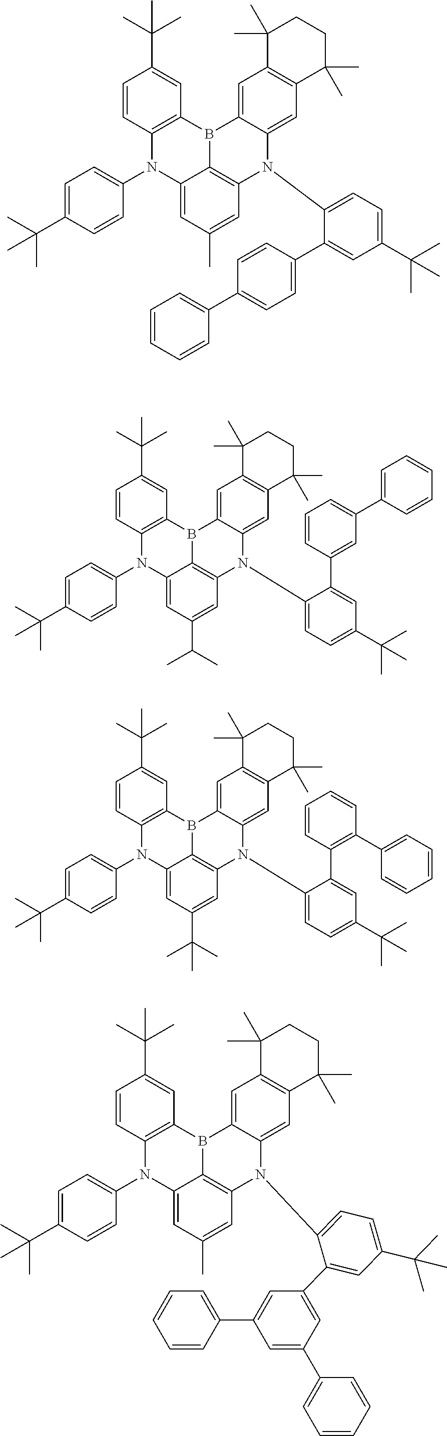

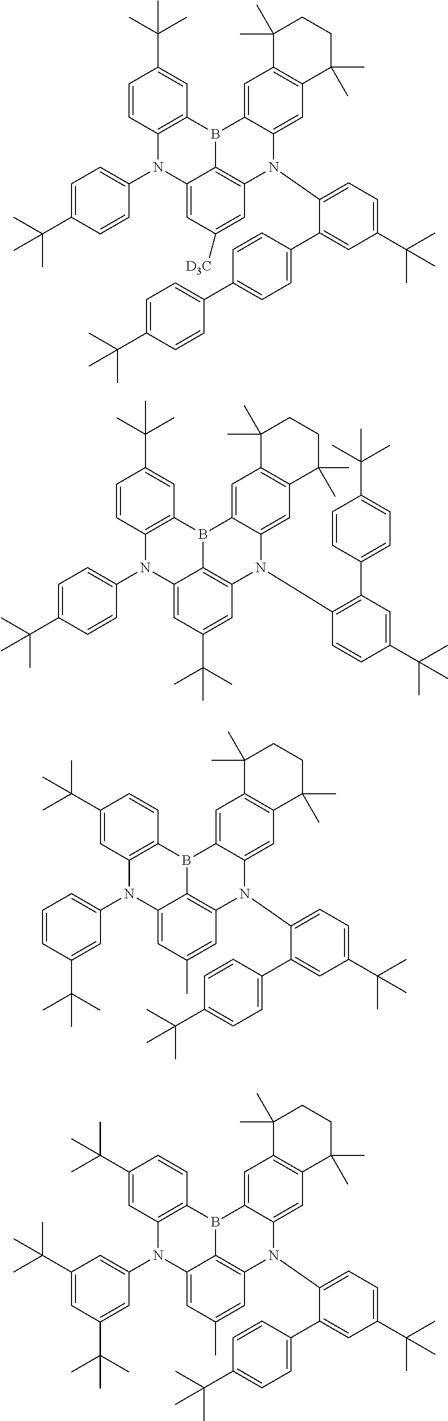

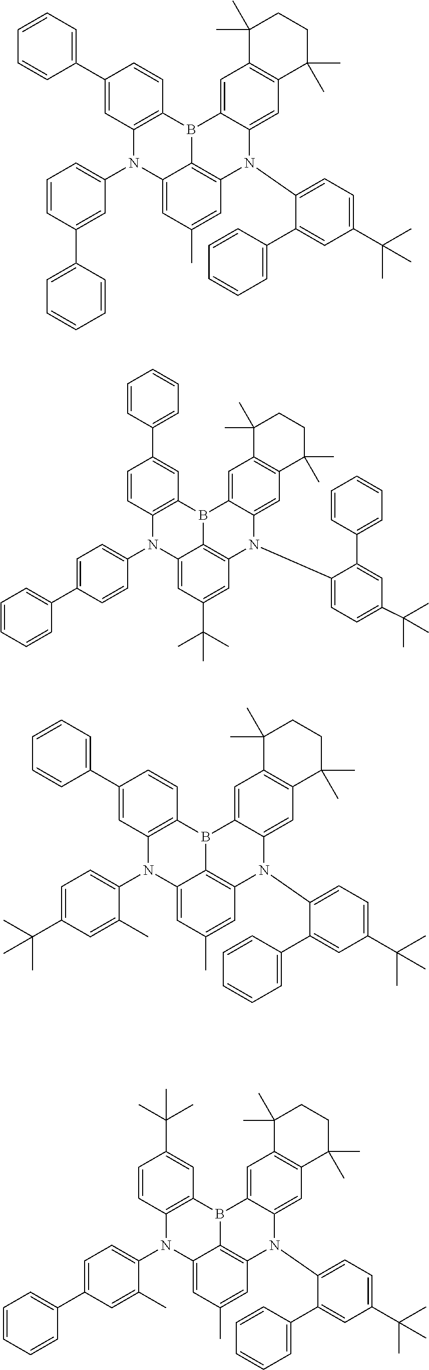

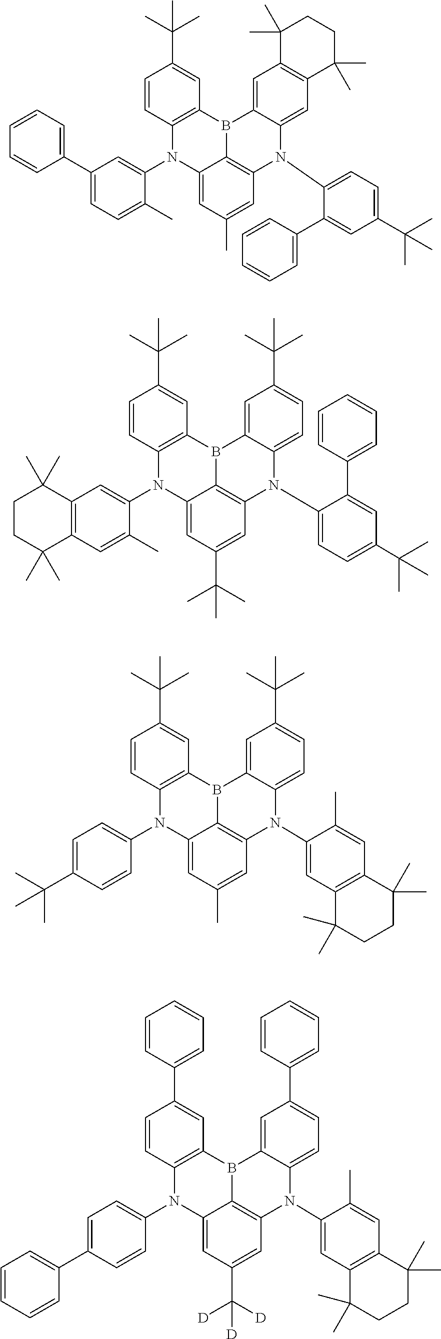

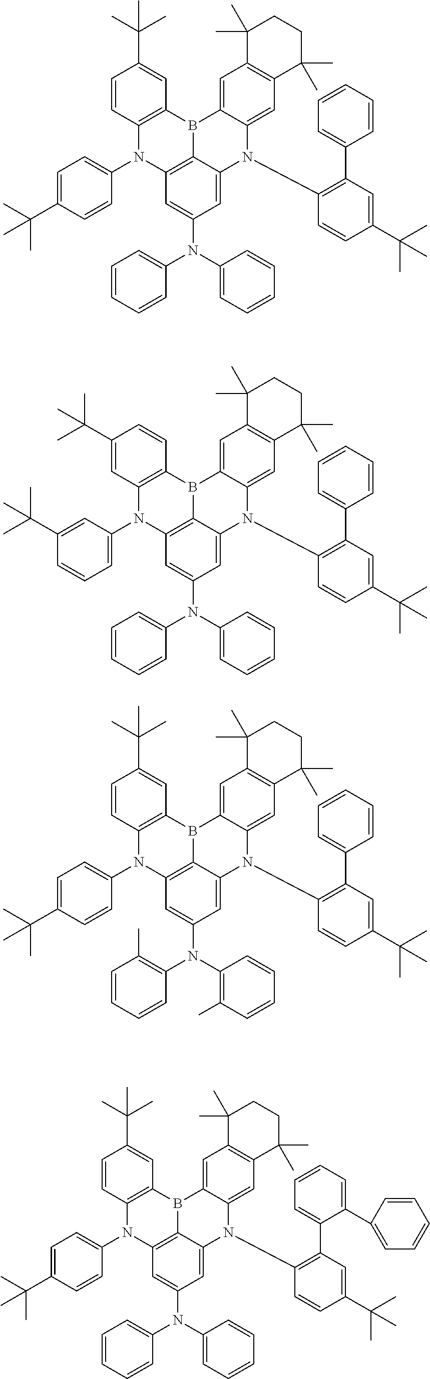

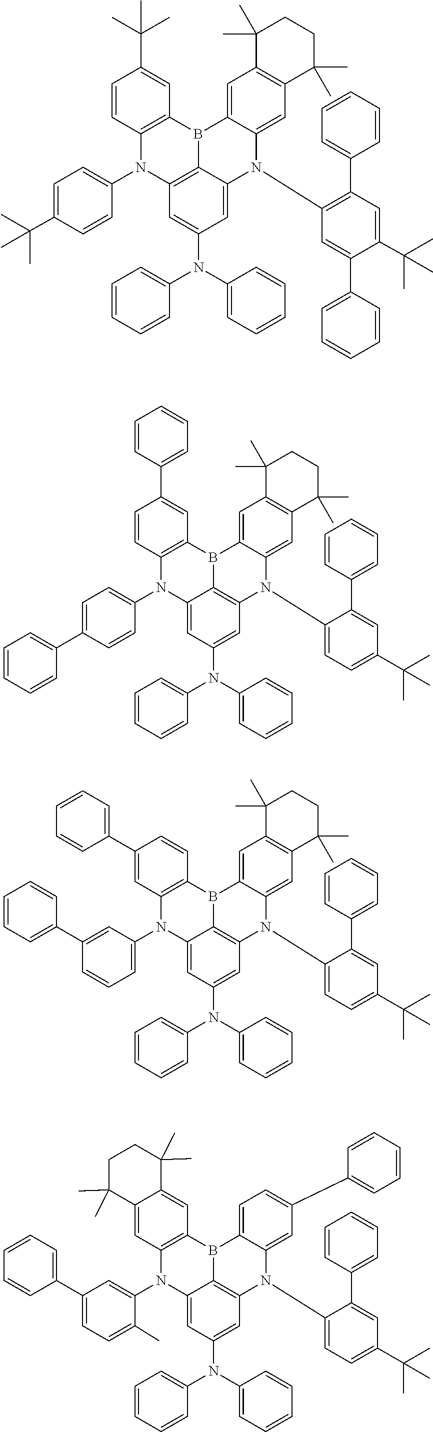

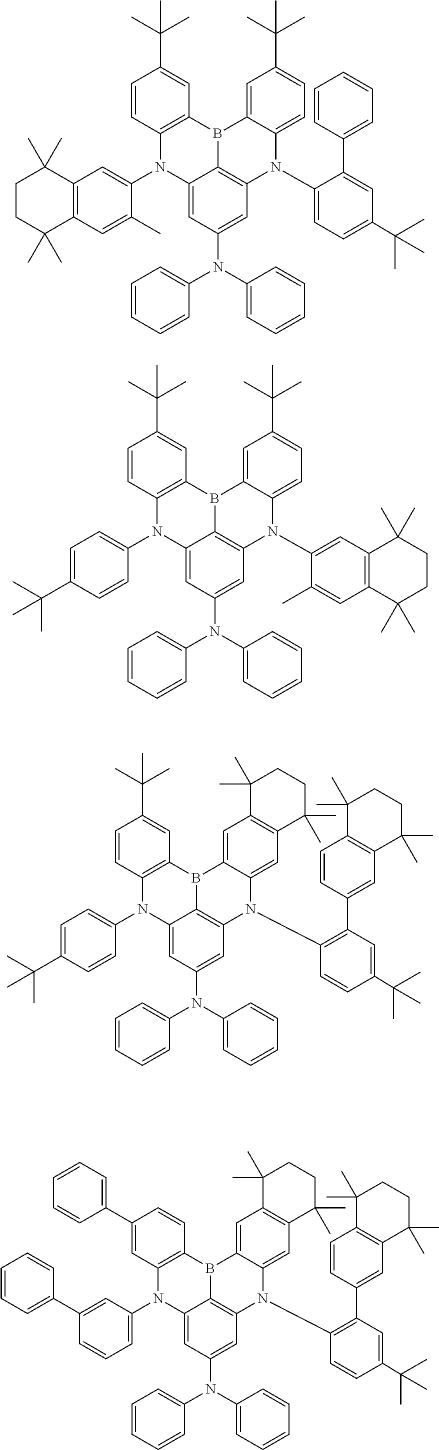

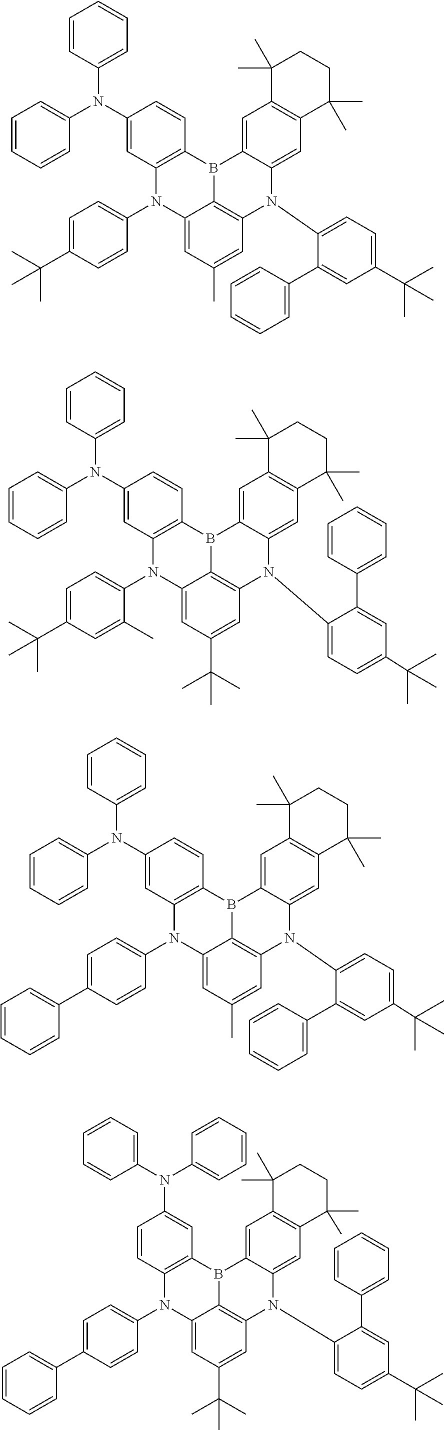

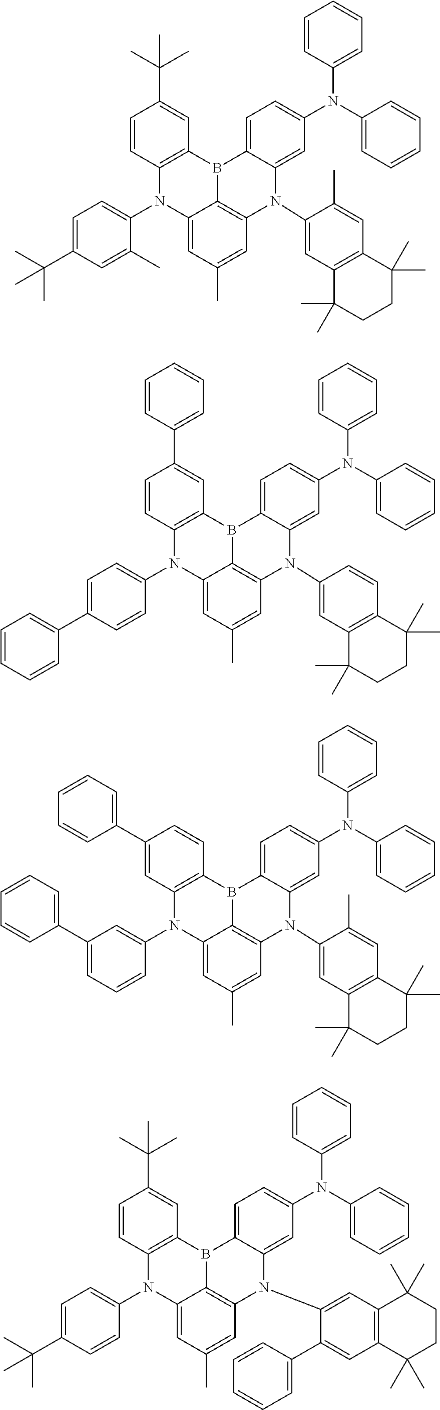

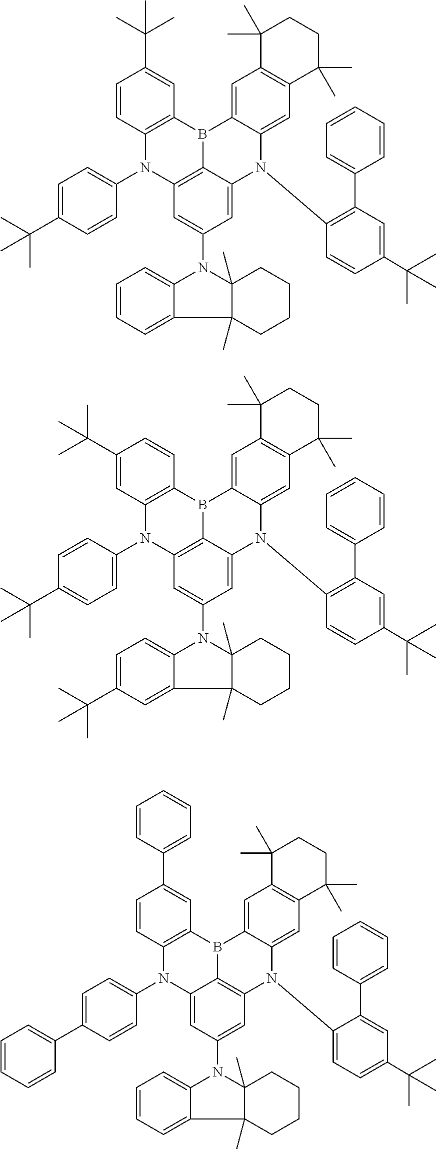

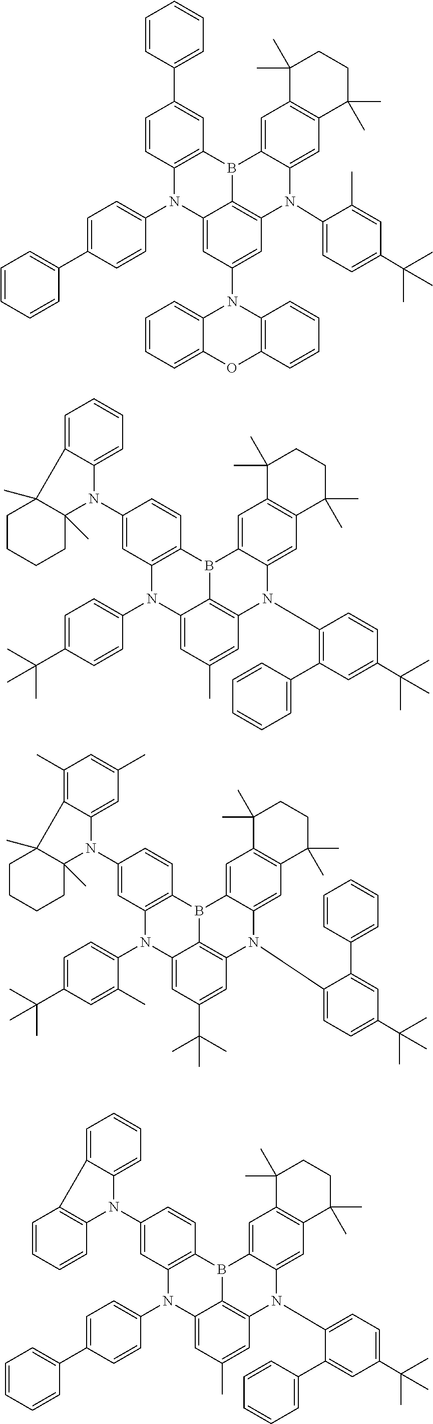

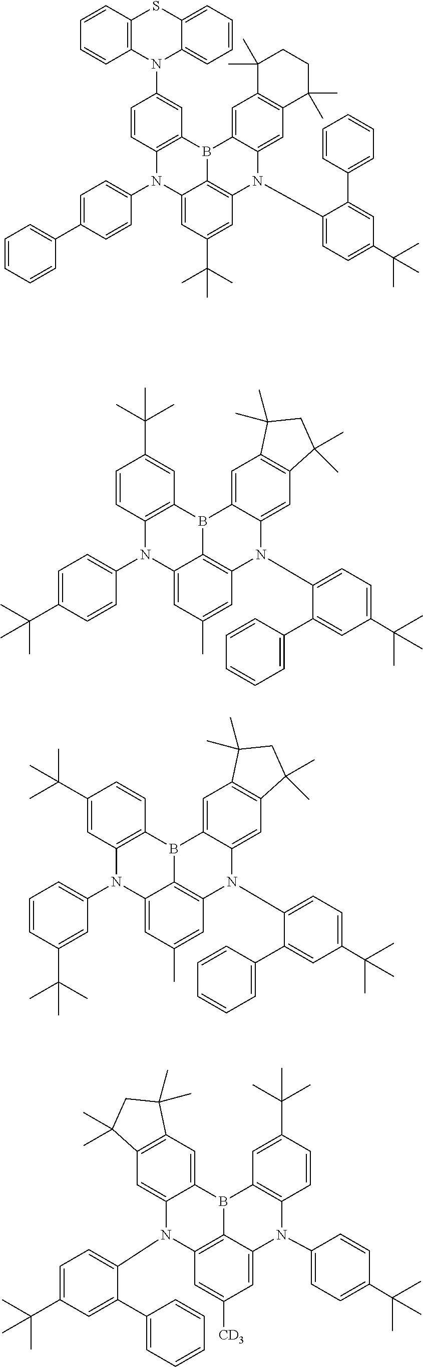

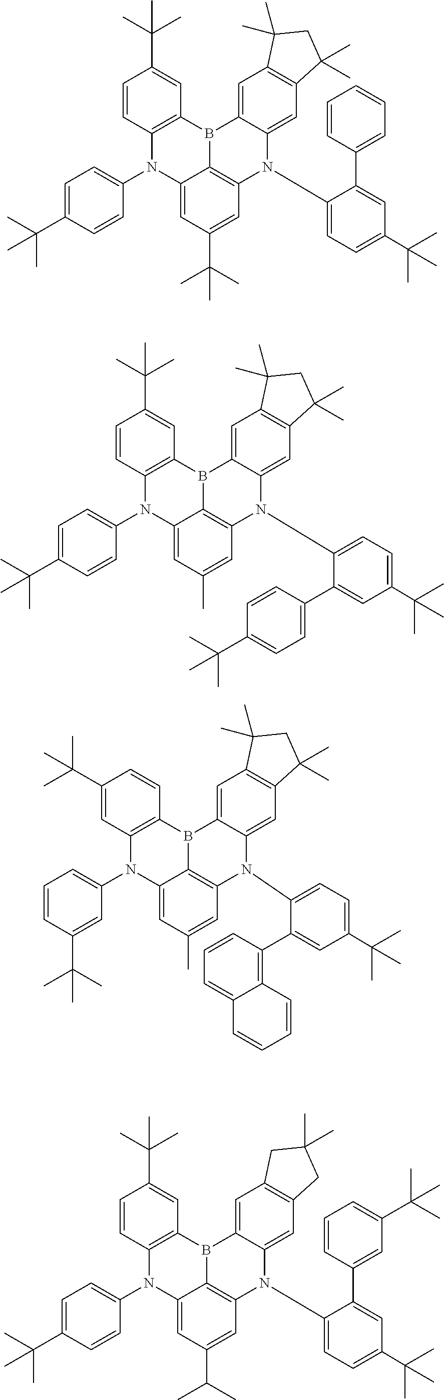

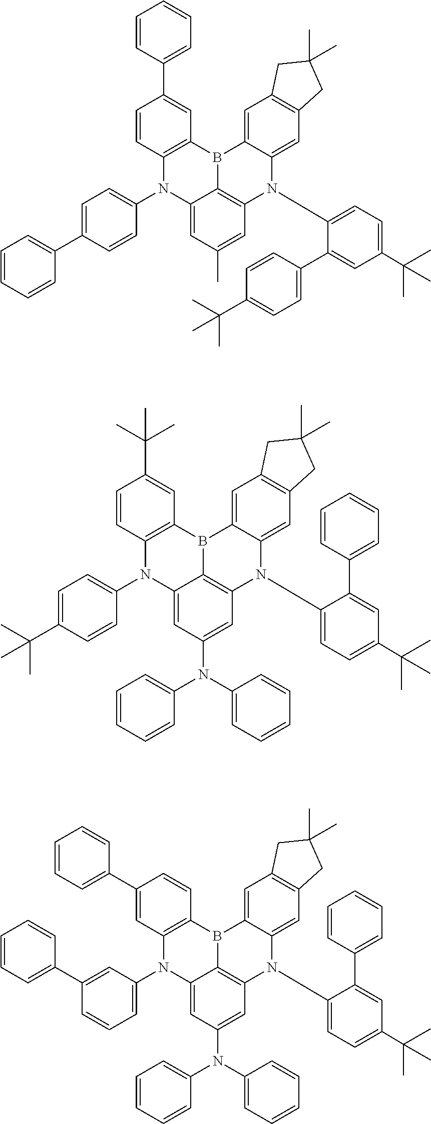

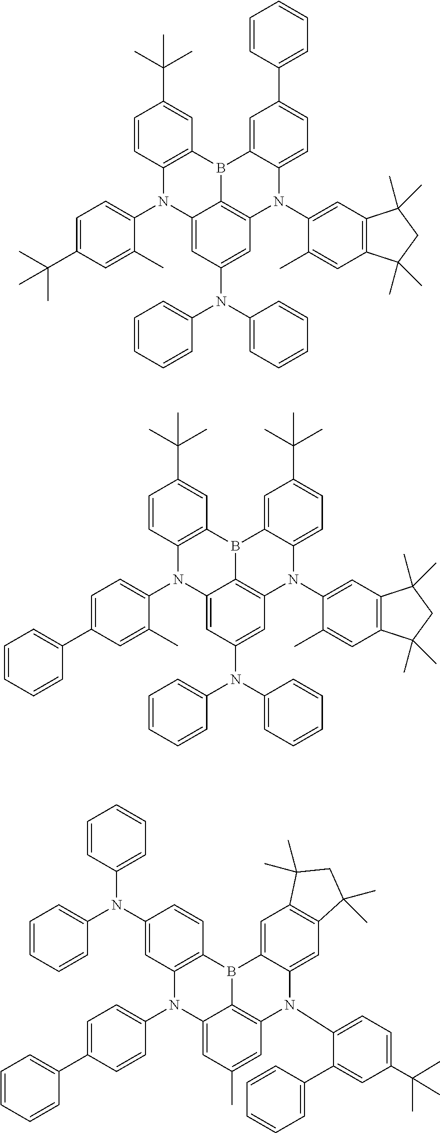 [SMILES: Cc1cc(C(C)(C)C)ccc1N1c2ccc(C(C)(C)C)cc2B2c3cc(-c4ccccc4)ccc3N(c3cc4c(cc3C)C(C)(C)CC4(C)C)c3cc(N(c4ccccc4)c4ccccc4)cc1c32 ; Cc1cc(-c2ccccc2)ccc1N1c2ccc(C(C)(C)C)cc2B2c3cc(C(C)(C)C)ccc3N(c3cc4c(cc3C)C(C)(C)CC4(C)C)c3cc(N(c4ccccc4)c4ccccc4)cc1c32 ; Cc1cc2c3c(c1)N(c1ccc(C(C)(C)C)cc1-c1ccccc1)c1cc4c(cc1B3c1ccc(N(c3ccccc3)c3ccccc3)cc1N2c1ccc(-c2ccccc2)cc1)C(C)(C)CC4(C)C]

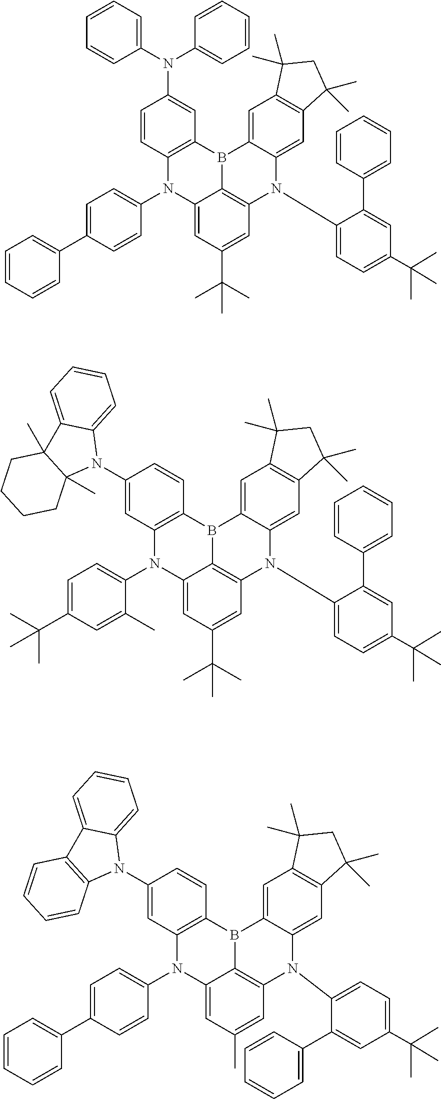 [SMILES: CC(C)(C)c1ccc(N2c3cc4c(cc3B3c5cc(N(c6ccccc6)c6ccccc6)ccc5N(c5ccc(-c6ccccc6)cc5)c5cc(C(C)(C)C)cc2c53)C(C)(C)CC4(C)C)c(-c2ccccc2)c1 ; Cc1cc(C(C)(C)C)ccc1N1c2cc(N3c4ccccc4C4(C)CCCCC34C)ccc2B2c3cc4c(cc3N(c3ccc(C(C)(C)C)cc3-c3ccccc3)c3cc(C(C)(C)C)cc1c32)C(C)(C)CC4(C)C ; Cc1cc2c3c(c1)N(c1ccc(C(C)(C)C)cc1-c1ccccc1)c1cc4c(cc1B3c1ccc(-n3c5ccccc5c5ccccc53)cc1N2c1ccc(-c2ccccc2)cc1)C(C)(C)CC4(C)C]

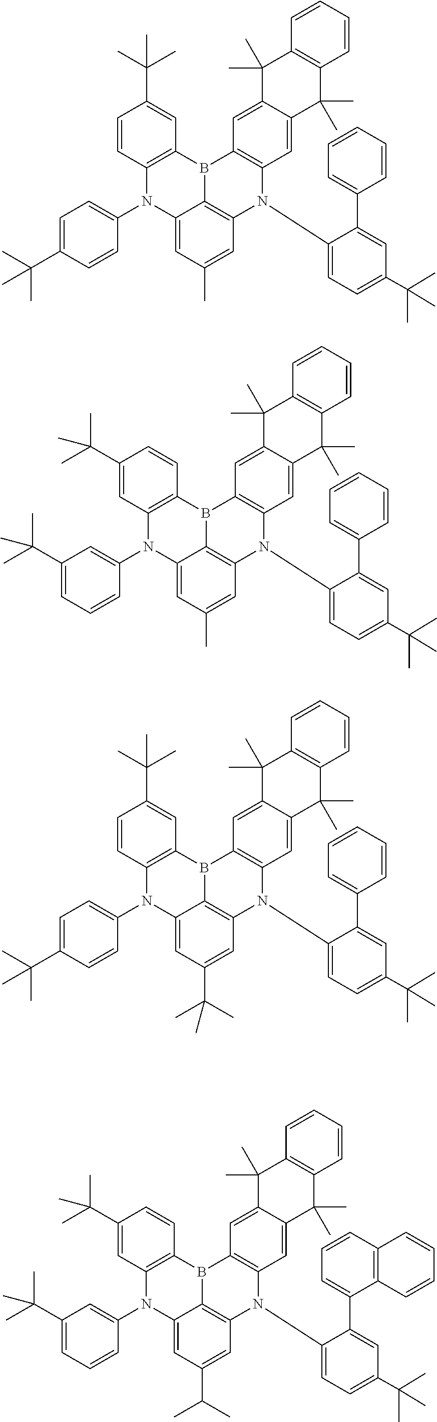

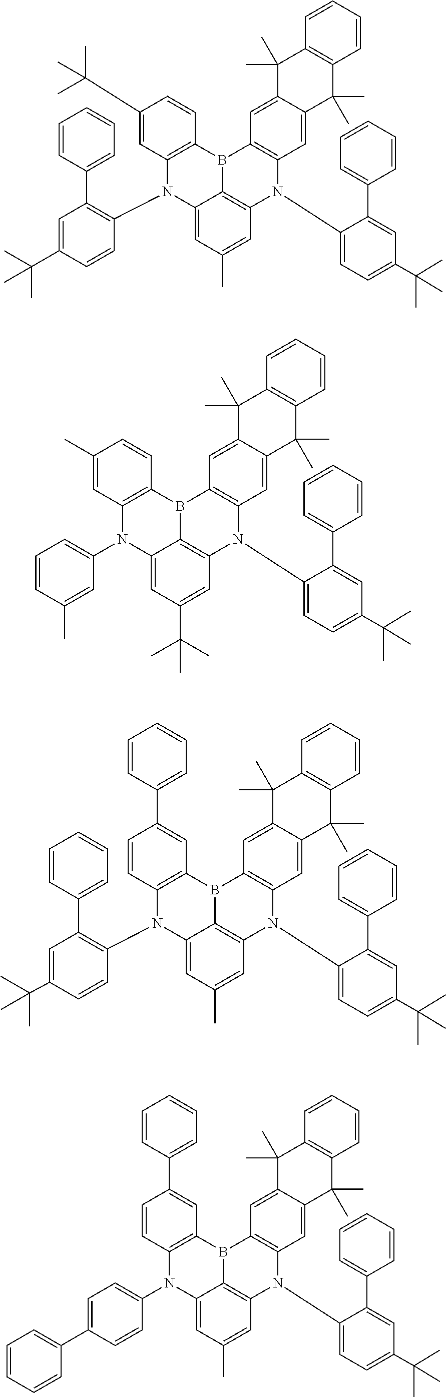

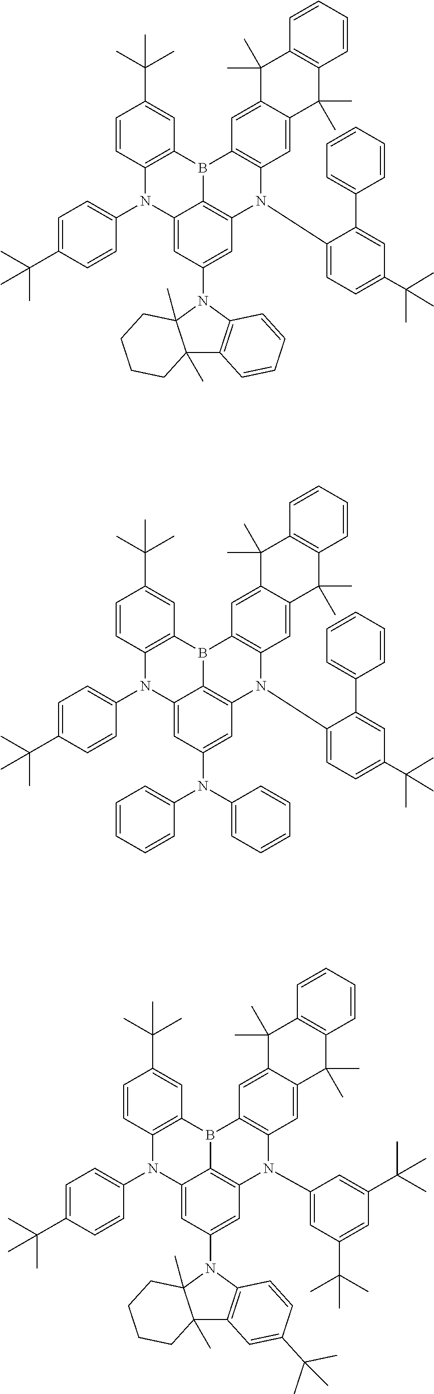

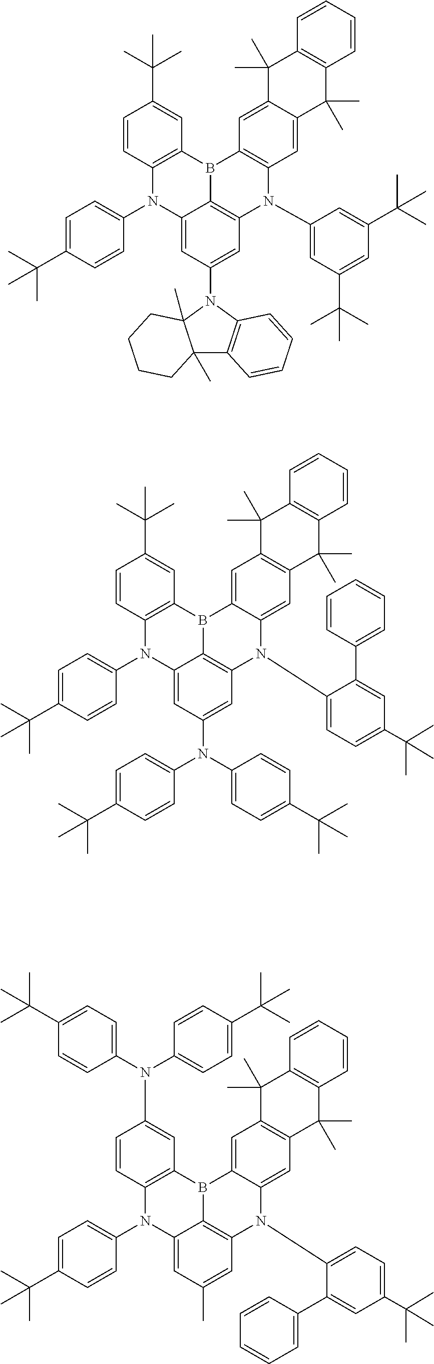

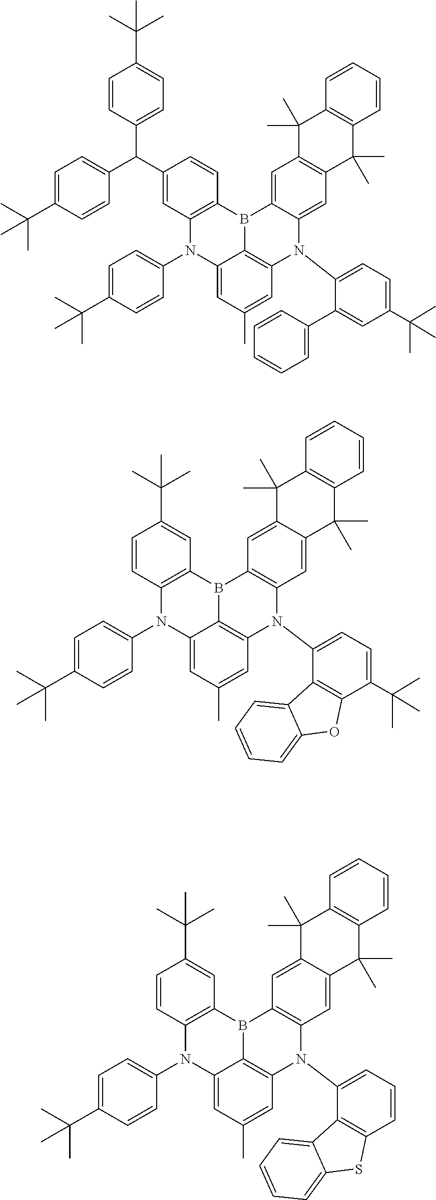

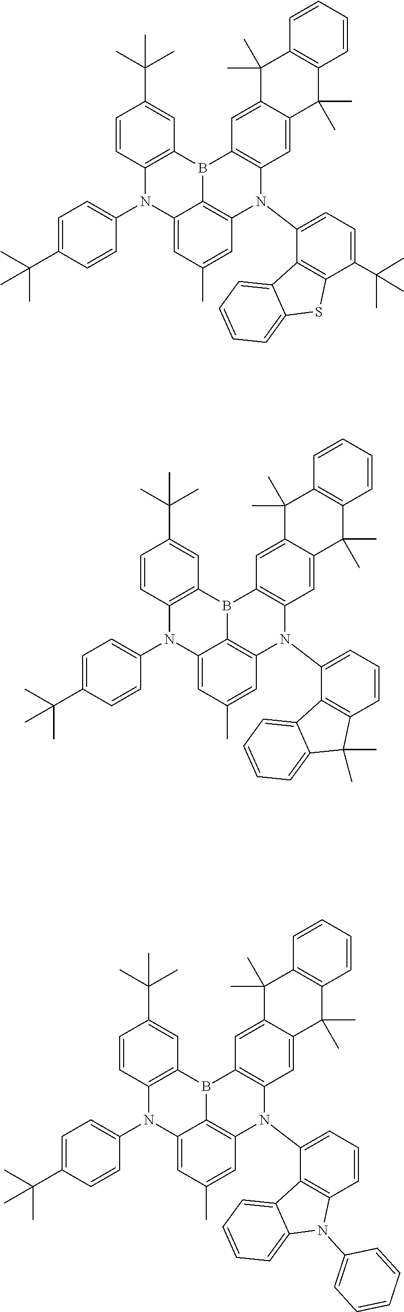

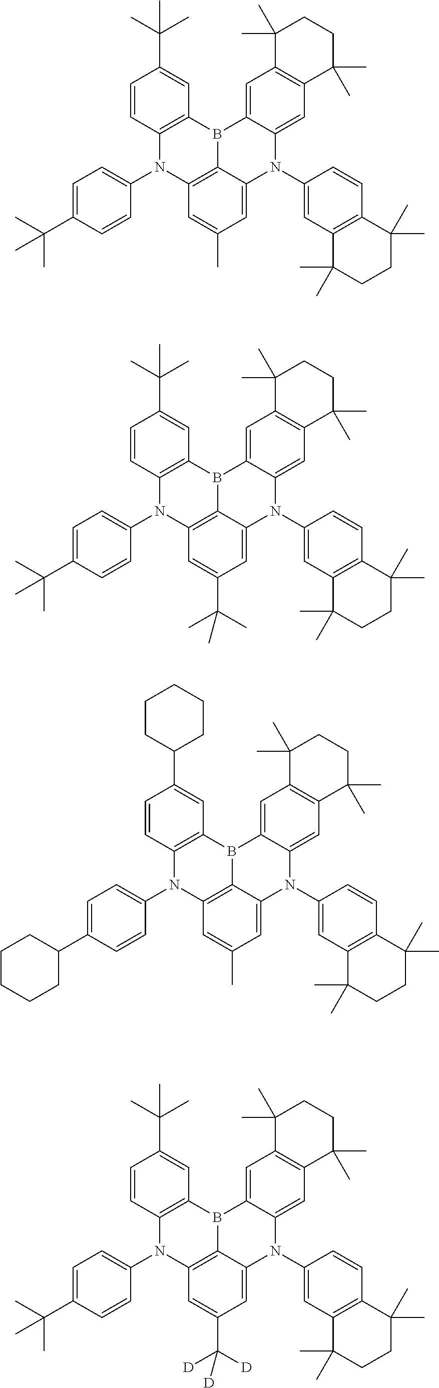

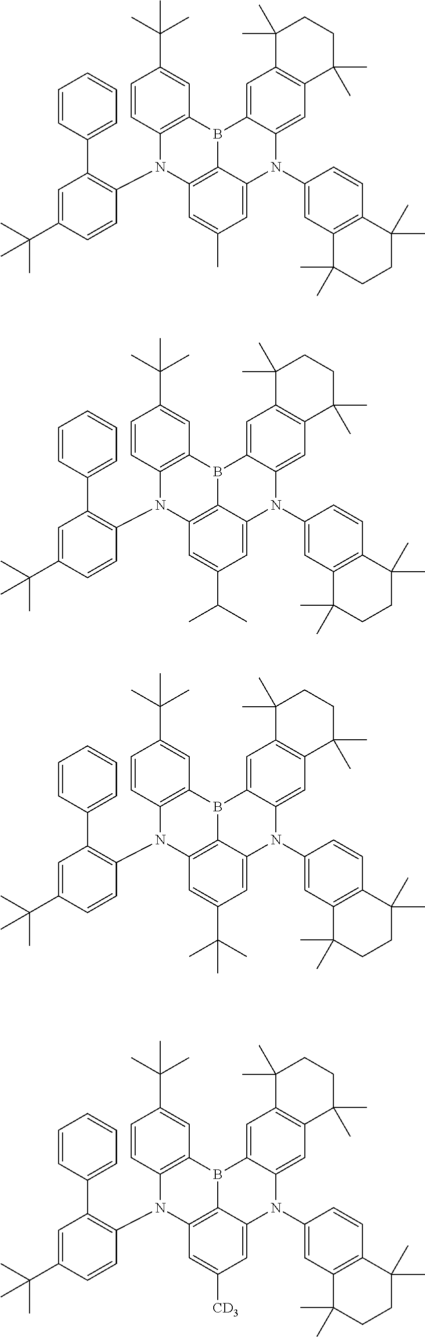

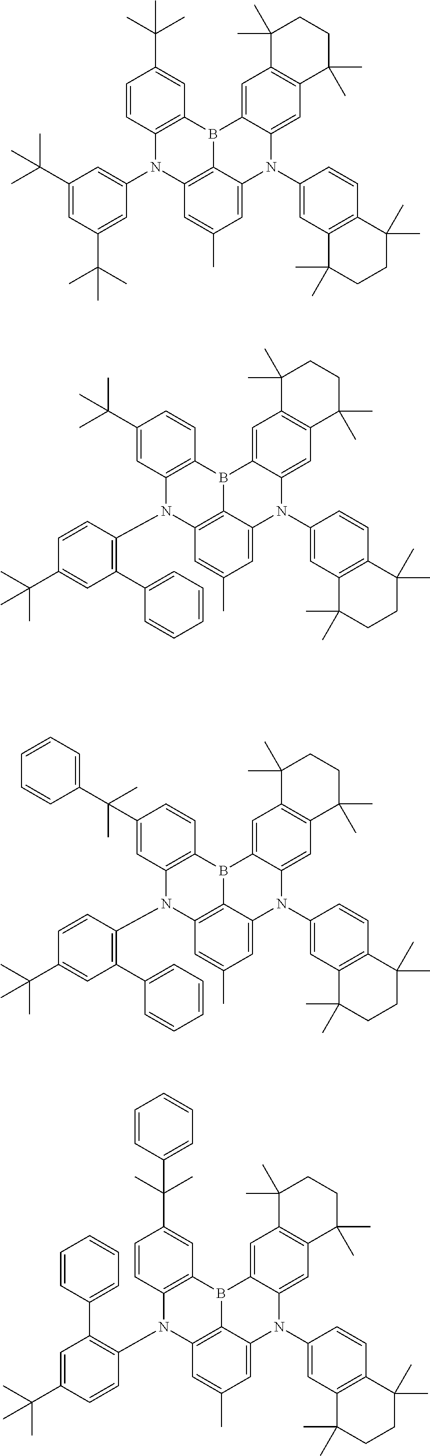

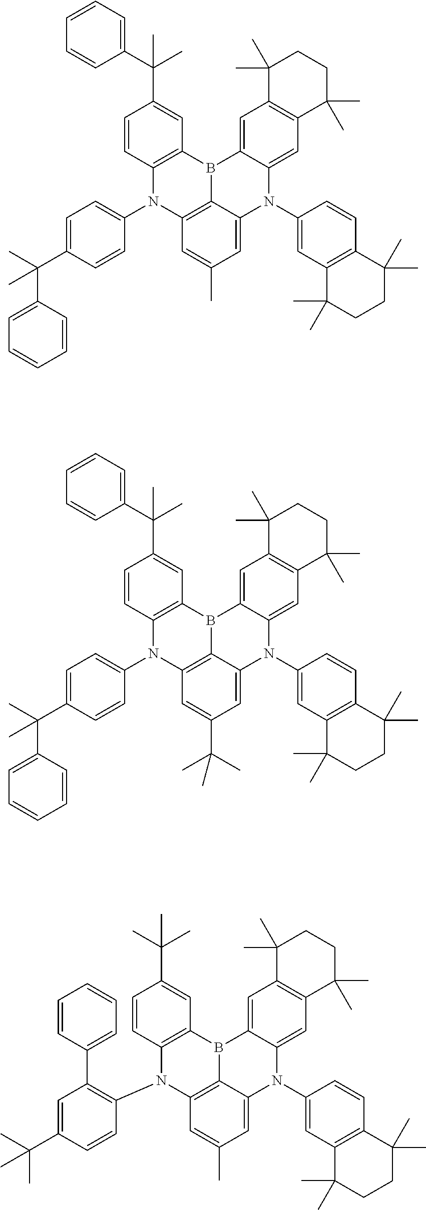

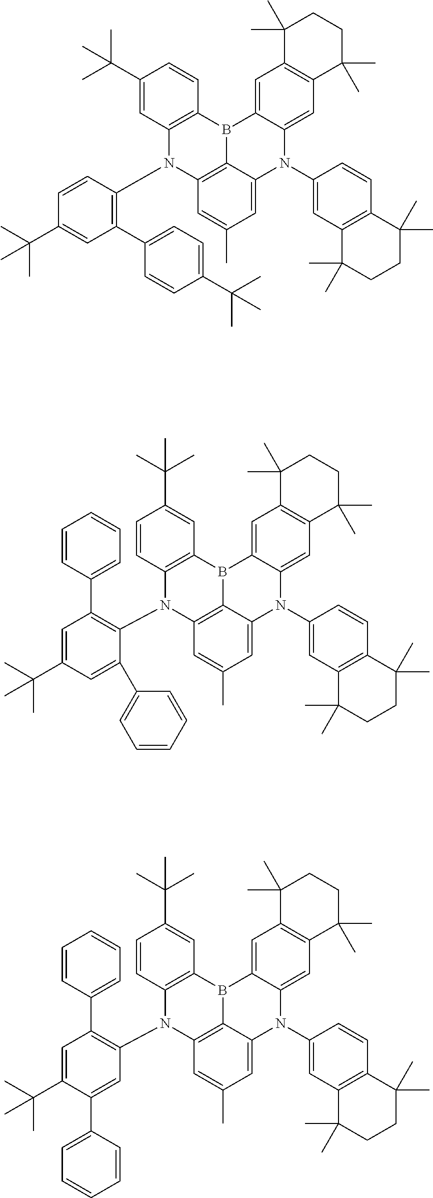

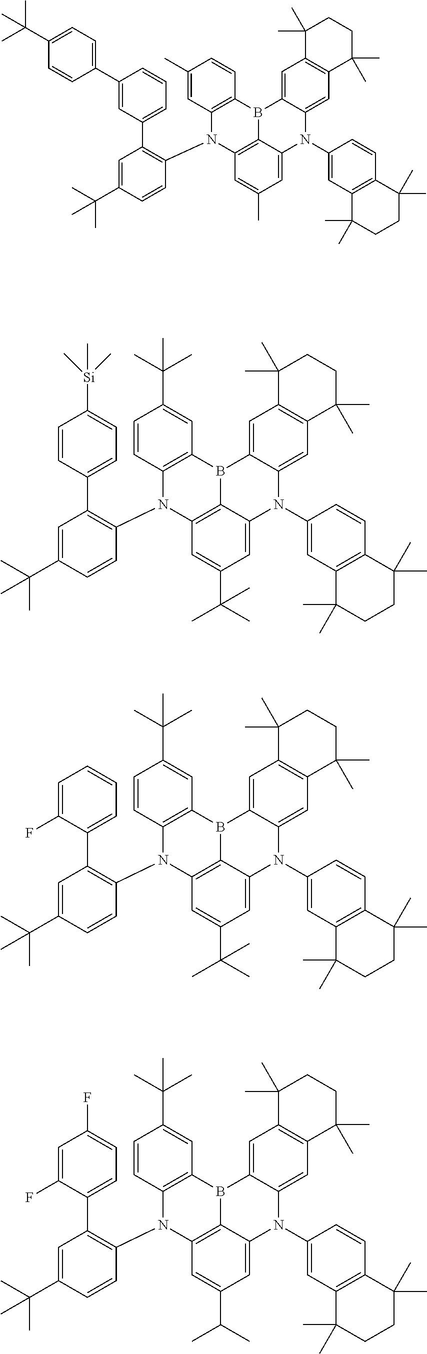

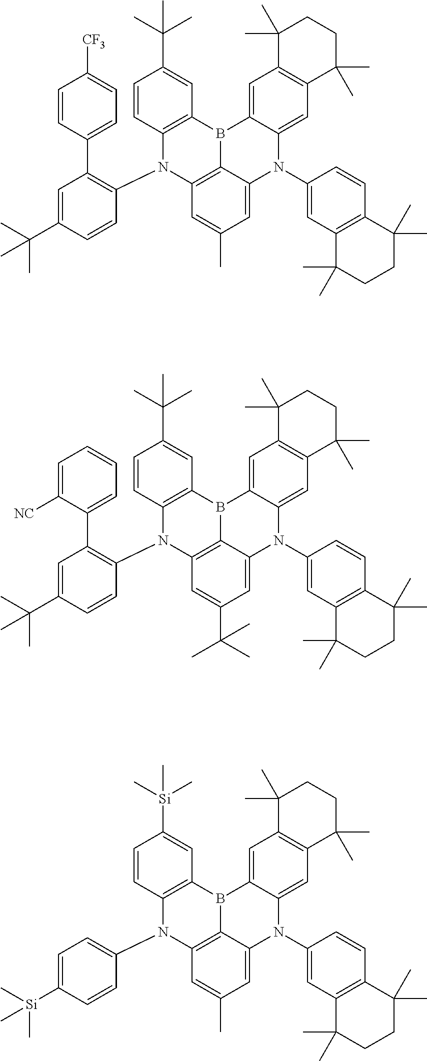

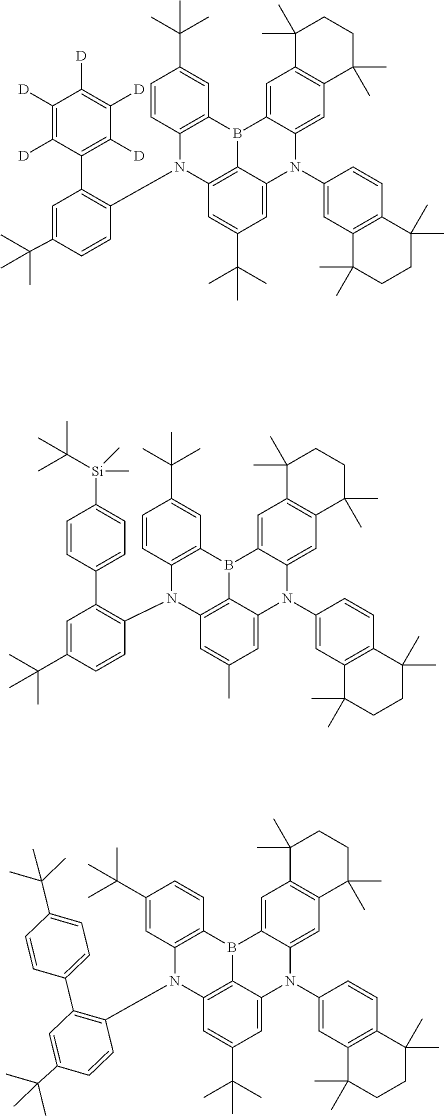

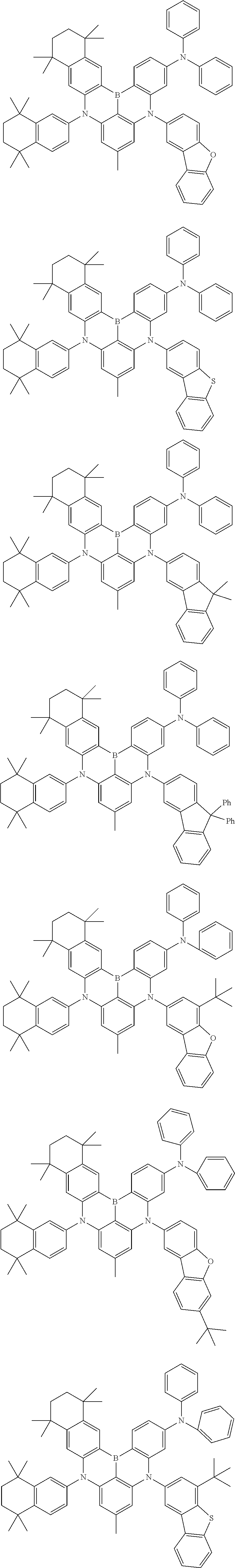

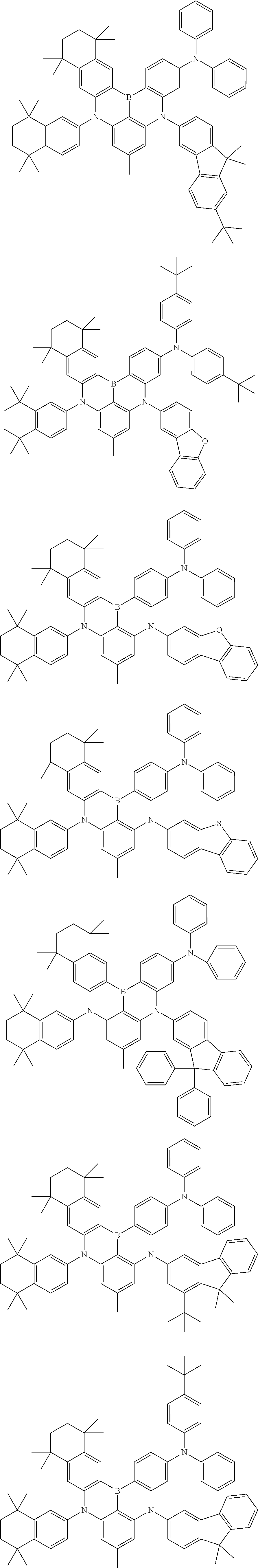

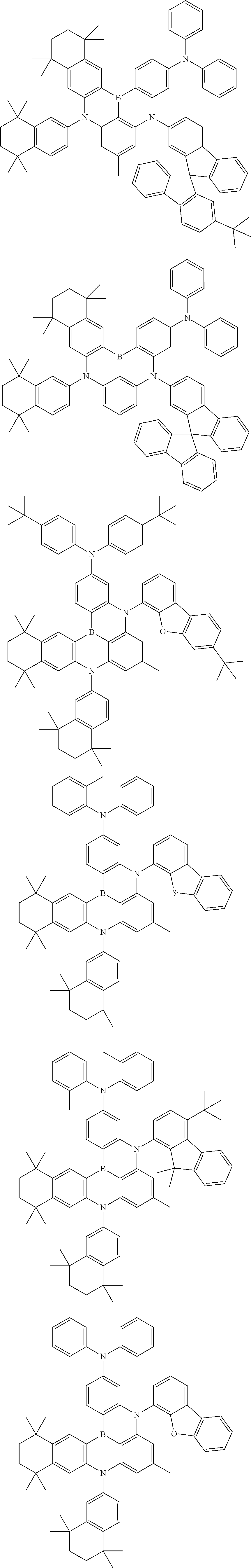

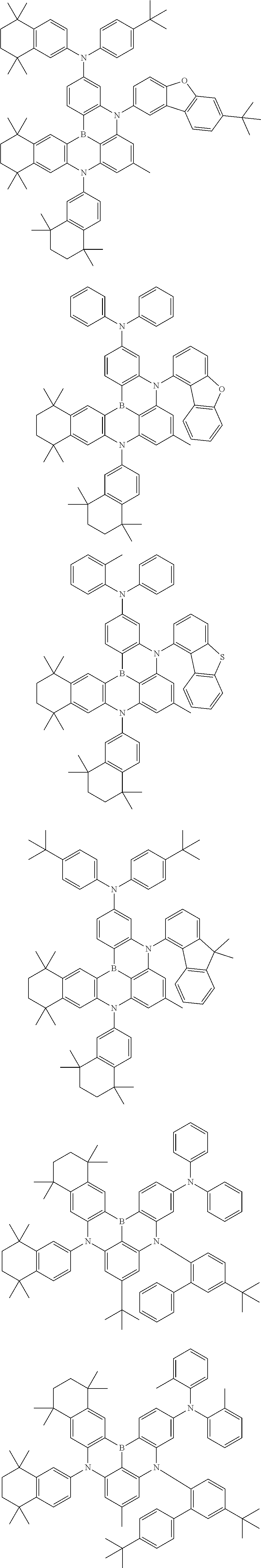

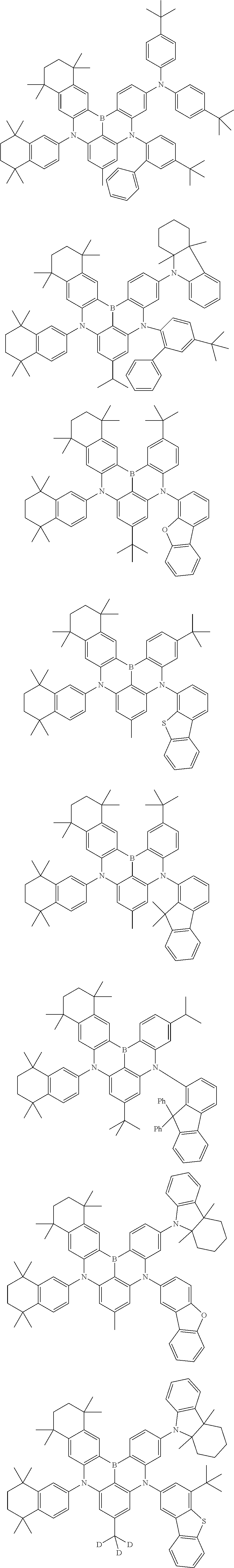

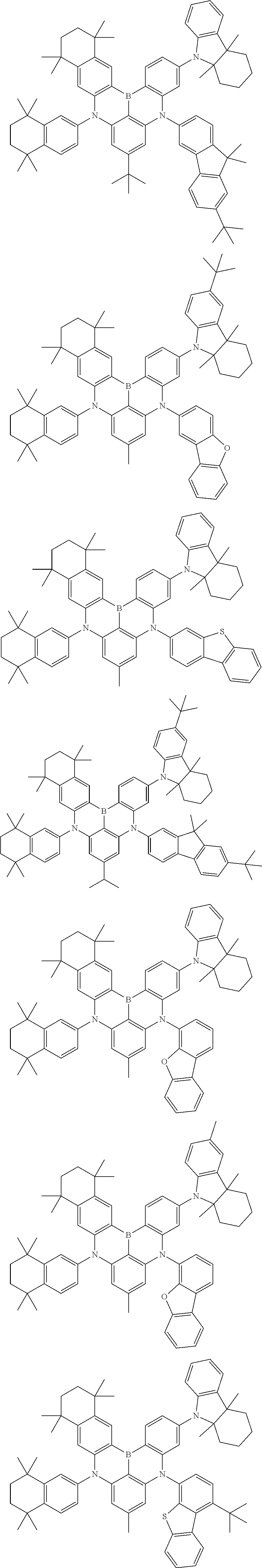

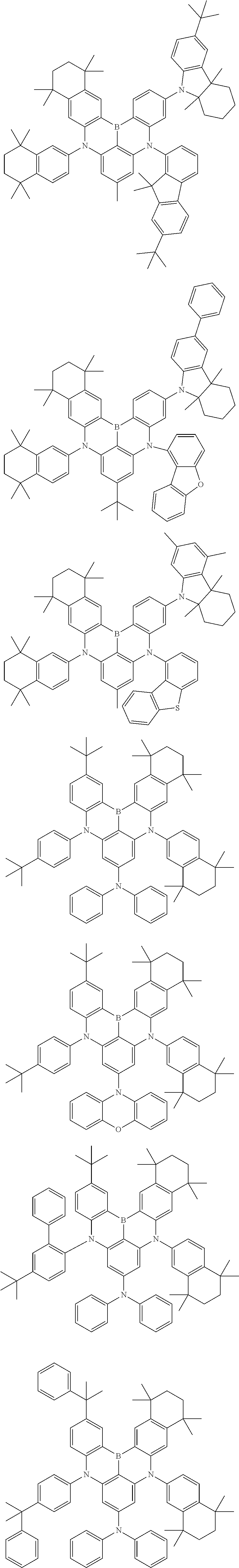

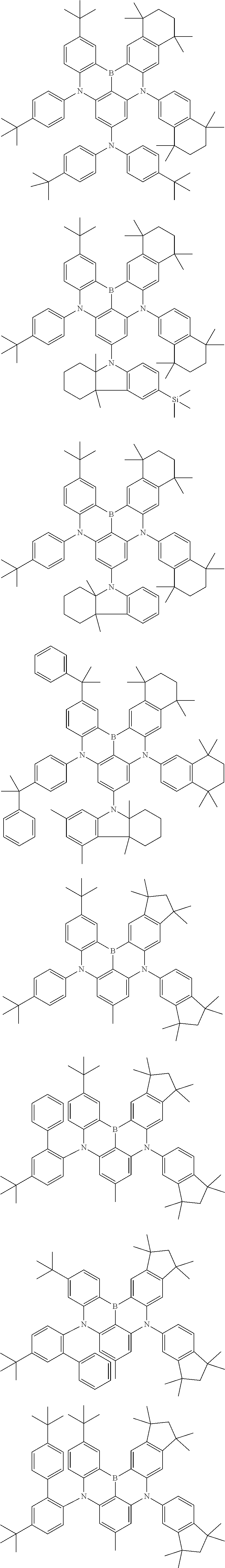

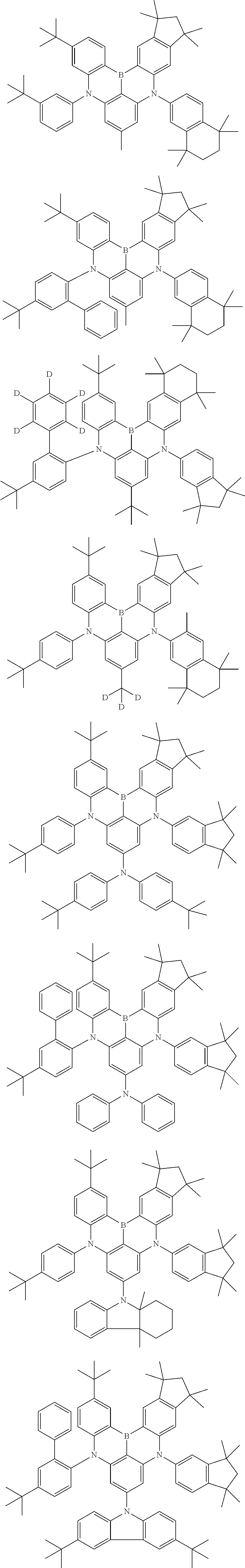

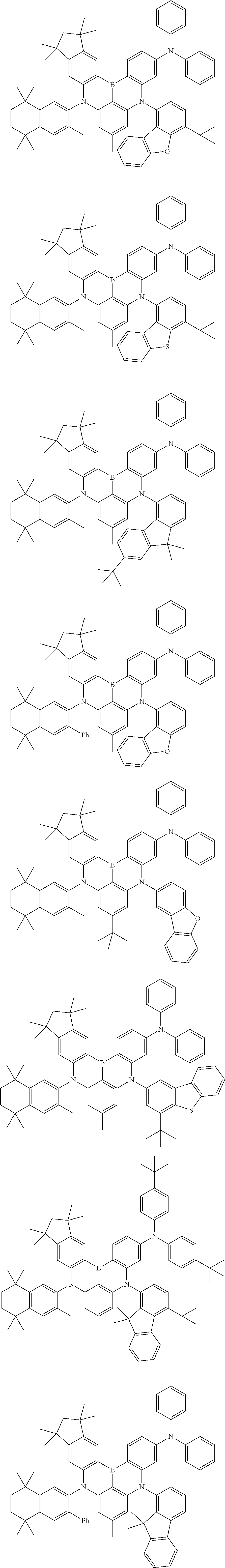

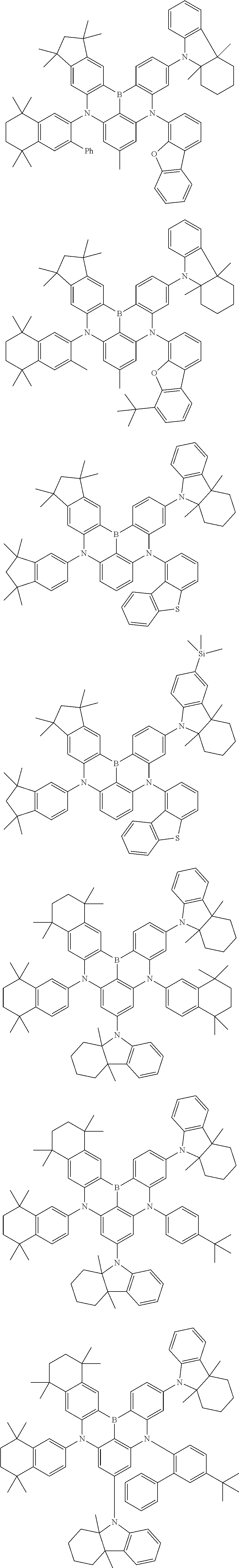

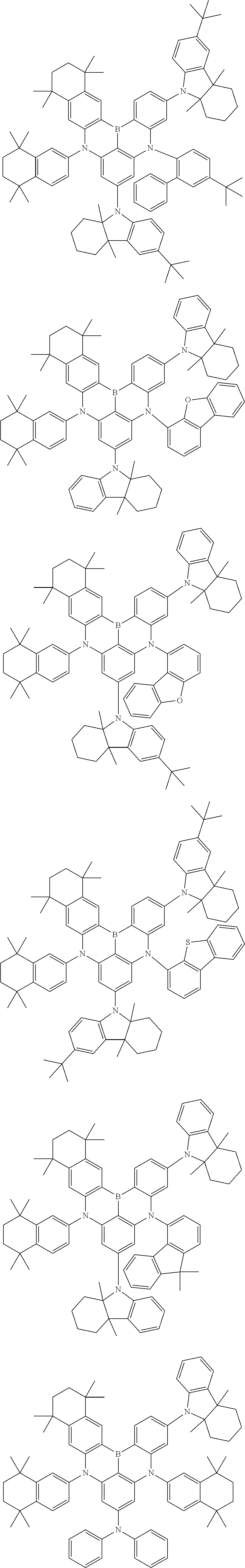

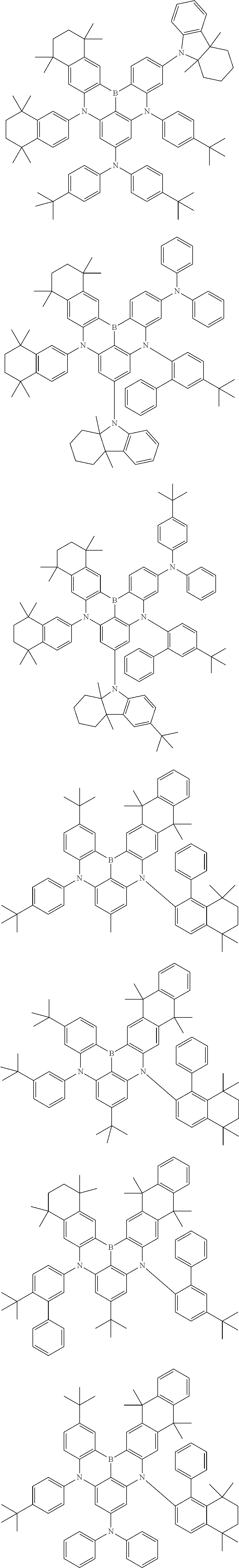

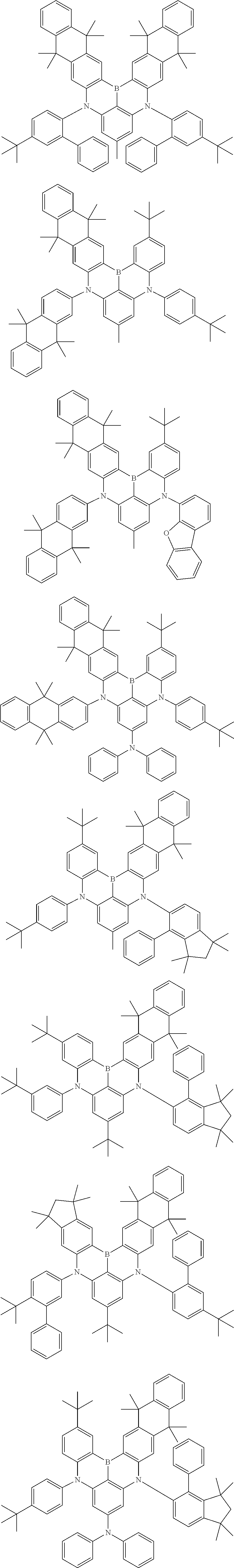

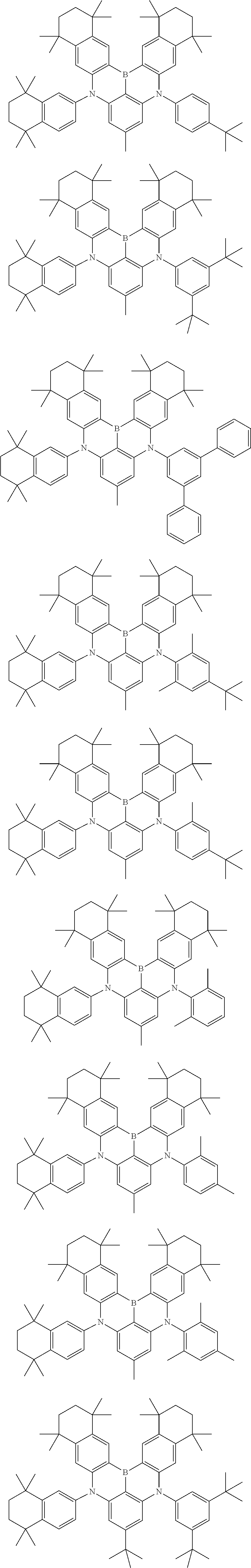

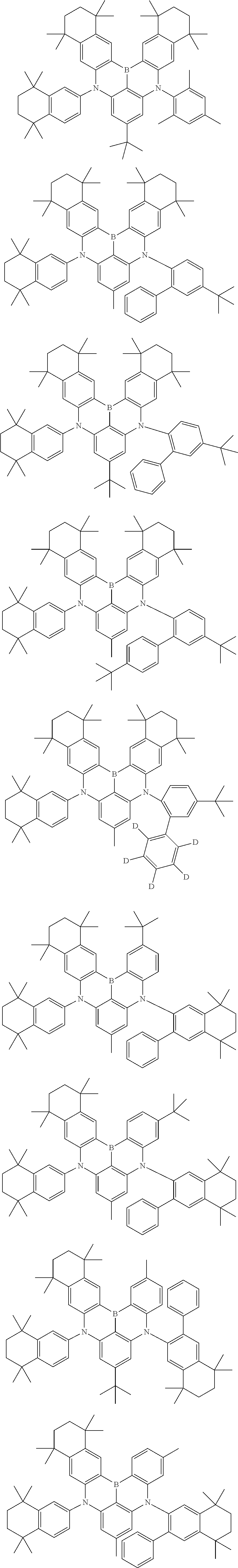

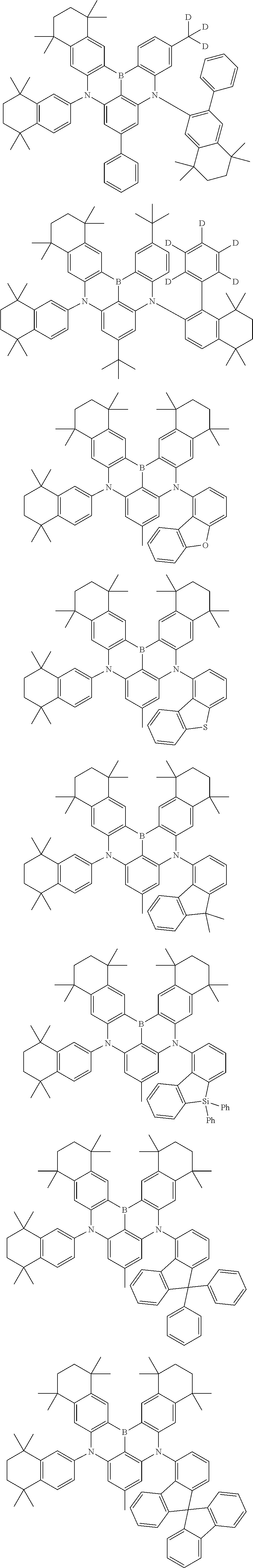

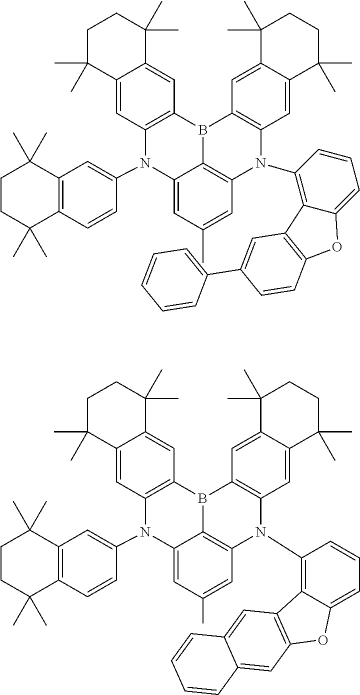 [SMILES: Cc1cc2c3c(c1)N(c1cccc4oc5ccc(-c6ccccc6)cc5c14)c1cc4c(cc1B3c1cc3c(cc1N2c1ccc2c(c1)C(C)(C)CCC2(C)C)C(C)(C)CCC3(C)C)C(C)(C)CCC4(C)C ; Cc1cc2c3c(c1)N(c1cccc4oc5cc6ccccc6cc5c14)c1cc4c(cc1B3c1cc3c(cc1N2c1ccc2c(c1)C(C)(C)CCC2(C)C)C(C)(C)CCC3(C)C)C(C)(C)CCC4(C)C]

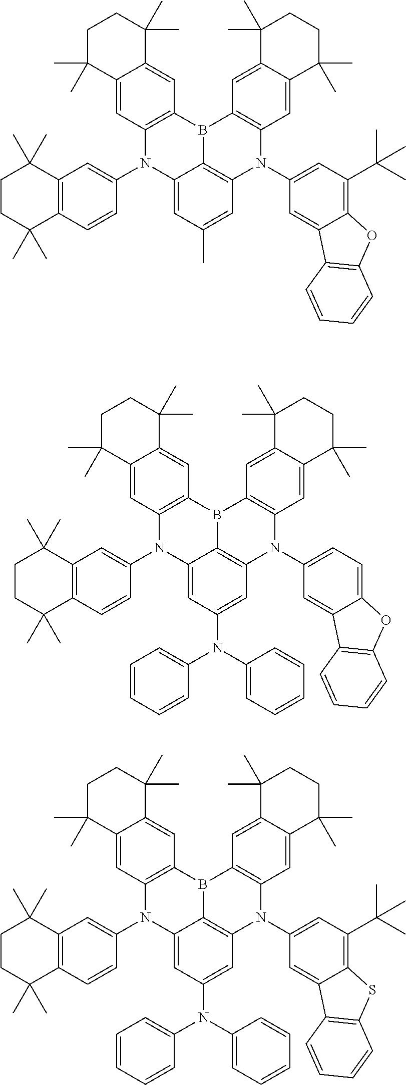

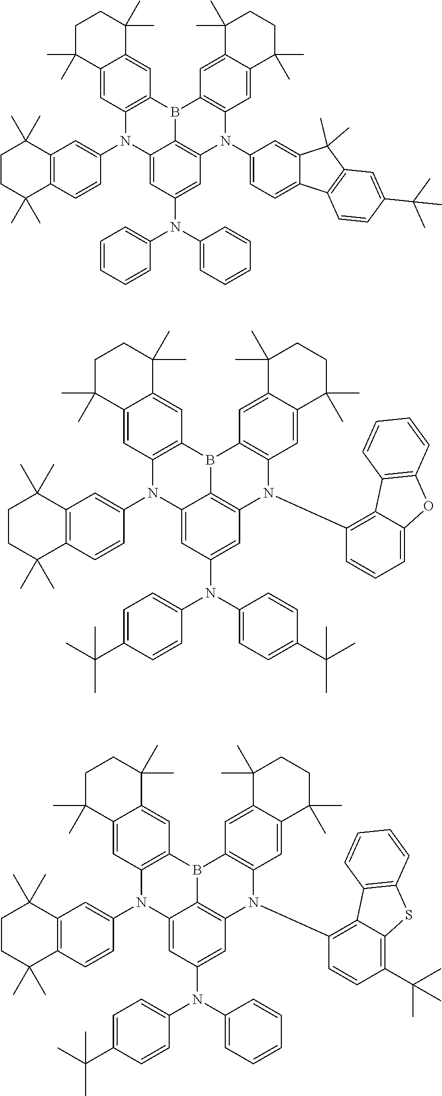 [SMILES: CC(C)(C)c1ccc2c(c1)C(C)(C)c1cc(N3c4cc5c(cc4B4c6cc7c(cc6N(c6ccc8c(c6)C(C)(C)CCC8(C)C)c6cc(N(c8ccccc8)c8ccccc8)cc3c64)C(C)(C)CCC7(C)C)C(C)(C)CCC5(C)C)ccc1-2 ; CC(C)(C)c1ccc(N(c2ccc(C(C)(C)C)cc2)c2cc3c4c(c2)N(c2cccc5oc6ccccc6c25)c2cc5c(cc2B4c2cc4c(cc2N3c2ccc3c(c2)C(C)(C)CCC3(C)C)C(C)(C)CCC4(C)C)C(C)(C)CCC5(C)C)cc1 ; CC(C)(C)c1ccc(N(c2ccccc2)c2cc3c4c(c2)N(c2ccc(C(C)(C)C)c5sc6ccccc6c25)c2cc5c(cc2B4c2cc4c(cc2N3c2ccc3c(c2)C(C)(C)CCC3(C)C)C(C)(C)CCC4(C)C)C(C)(C)CCC5(C)C)cc1]

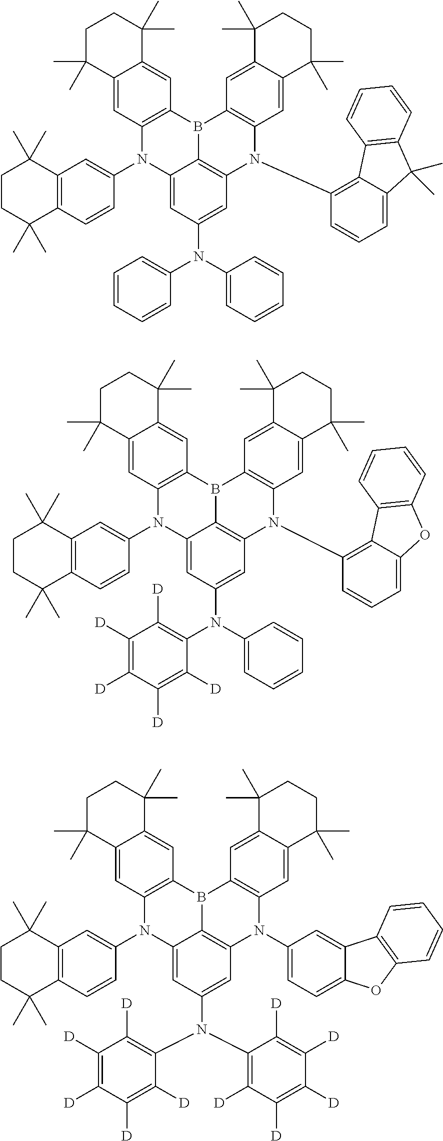

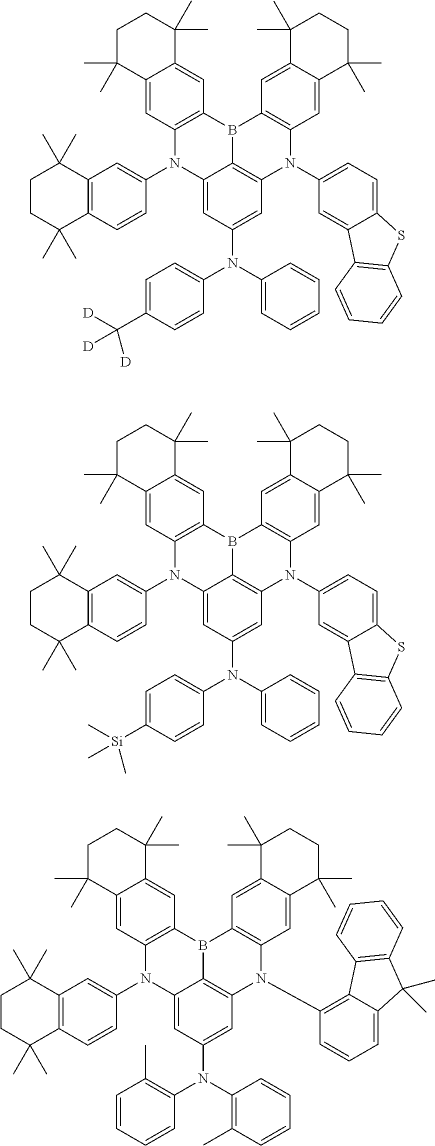 [SMILES: [2H]C([2H])([2H])c1ccc(N(c2ccccc2)c2cc3c4c(c2)N(c2ccc5sc6ccccc6c5c2)c2cc5c(cc2B4c2cc4c(cc2N3c2ccc3c(c2)C(C)(C)CCC3(C)C)C(C)(C)CCC4(C)C)C(C)(C)CCC5(C)C)cc1 ; CC1(C)CCC(C)(C)c2cc(N3c4cc5c(cc4B4c6cc7c(cc6N(c6ccc8sc9ccccc9c8c6)c6cc(N(c8ccccc8)c8ccc([Si](C)(C)C)cc8)cc3c64)C(C)(C)CCC7(C)C)C(C)(C)CCC5(C)C)ccc21 ; Cc1ccccc1N(c1cc2c3c(c1)N(c1cccc4c1-c1ccccc1C4(C)C)c1cc4c(cc1B3c1cc3c(cc1N2c1ccc2c(c1)C(C)(C)CCC2(C)C)C(C)(C)CCC3(C)C)C(C)(C)CCC4(C)C)c1ccccc1C]

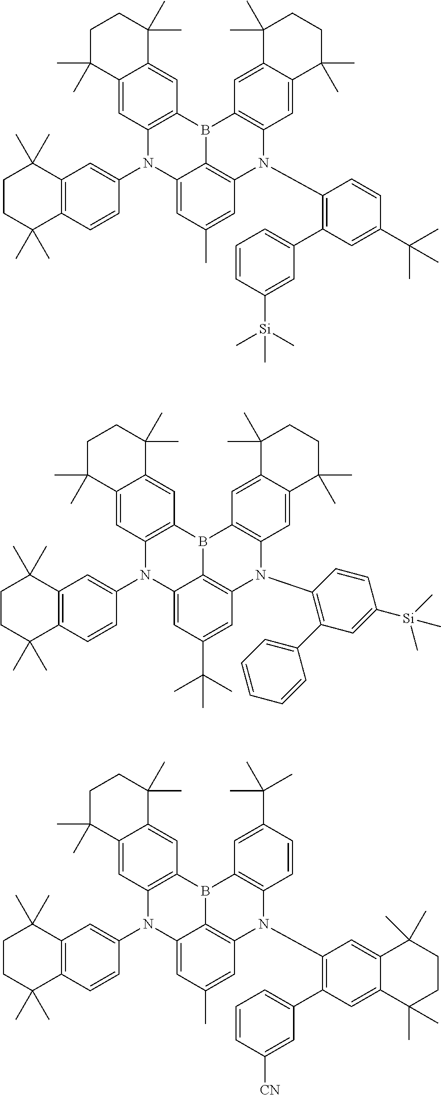 [SMILES: Cc1cc2c3c(c1)N(c1ccc(C(C)(C)C)cc1-c1cccc([Si](C)(C)C)c1)c1cc4c(cc1B3c1cc3c(cc1N2c1ccc2c(c1)C(C)(C)CCC2(C)C)C(C)(C)CCC3(C)C)C(C)(C)CCC4(C)C ; CC(C)(C)c1cc2c3c(c1)N(c1ccc([Si](C)(C)C)cc1-c1ccccc1)c1cc4c(cc1B3c1cc3c(cc1N2c1ccc2c(c1)C(C)(C)CCC2(C)C)C(C)(C)CCC3(C)C)C(C)(C)CCC4(C)C ; Cc1cc2c3c(c1)N(c1cc4c(cc1-c1cccc(C#N)c1)C(C)(C)CCC4(C)C)c1ccc(C(C)(C)C)cc1B3c1cc3c(cc1N2c1ccc2c(c1)C(C)(C)CCC2(C)C)C(C)(C)CCC3(C)C]

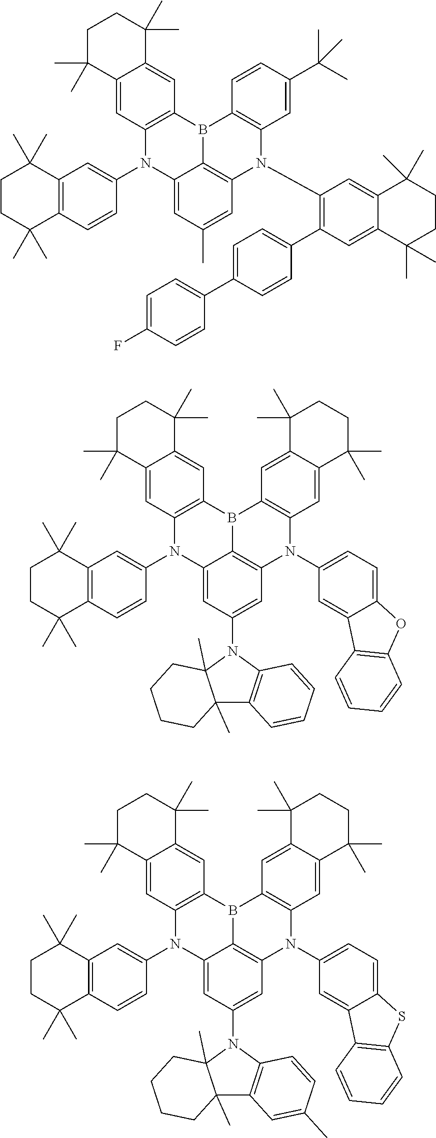

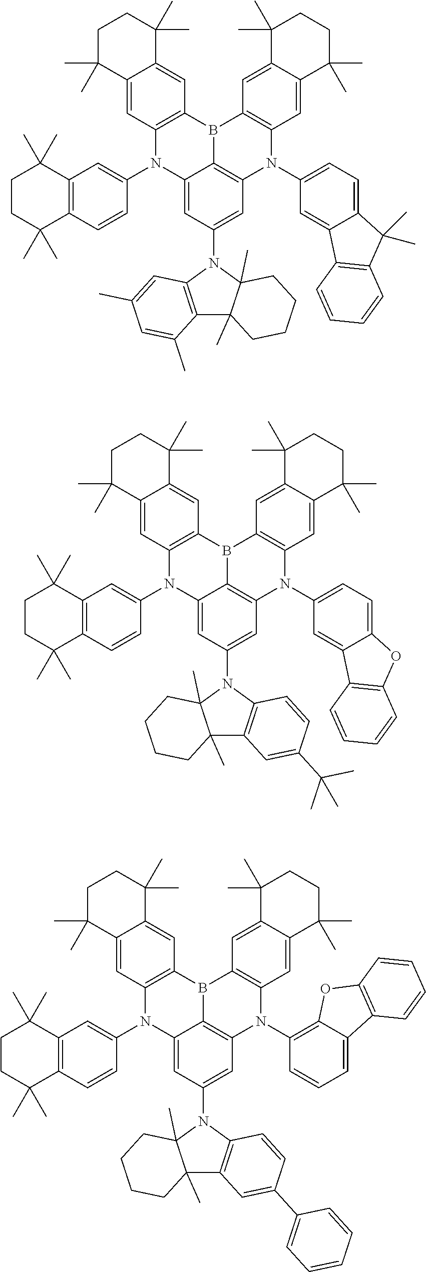 [SMILES: Cc1cc(C)c2c(c1)N(c1cc3c4c(c1)N(c1ccc5c(c1)C(C)(C)CCC5(C)C)c1cc5c(cc1B4c1cc4c(cc1N3c1ccc3c(c1)-c1ccccc1C3(C)C)C(C)(C)CCC4(C)C)C(C)(C)CCC5(C)C)C1(C)CCCCC21C ; CC(C)(C)c1ccc2c(c1)C1(C)CCCCC1(C)N2c1cc2c3c(c1)N(c1ccc4oc5ccccc5c4c1)c1cc4c(cc1B3c1cc3c(cc1N2c1ccc2c(c1)C(C)(C)CCC2(C)C)C(C)(C)CCC3(C)C)C(C)(C)CCC4(C)C ; CC1(C)CCC(C)(C)c2cc(N3c4cc5c(cc4B4c6cc7c(cc6N(c6cccc8c6oc6ccccc68)c6cc(N8c9ccc(-c%10ccccc%10)cc9C9(C)CCCCC89C)cc3c64)C(C)(C)CCC7(C)C)C(C)(C)CCC5(C)C)ccc21]

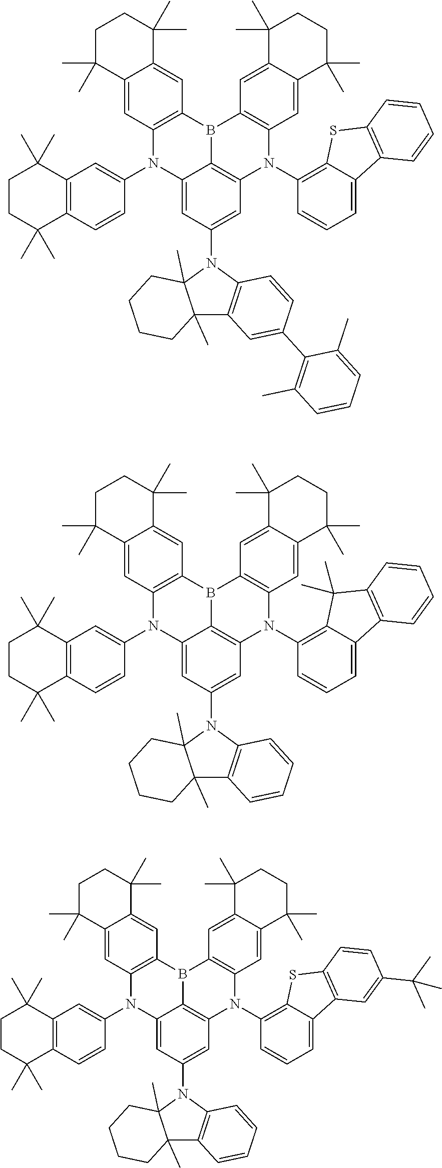 [SMILES: Cc1cccc(C)c1-c1ccc2c(c1)C1(C)CCCCC1(C)N2c1cc2c3c(c1)N(c1cccc4c1sc1ccccc14)c1cc4c(cc1B3c1cc3c(cc1N2c1ccc2c(c1)C(C)(C)CCC2(C)C)C(C)(C)CCC3(C)C)C(C)(C)CCC4(C)C ; CC1(C)CCC(C)(C)c2cc(N3c4cc5c(cc4B4c6cc7c(cc6N(c6cccc8c6C(C)(C)c6ccccc6-8)c6cc(N8c9ccccc9C9(C)CCCCC89C)cc3c64)C(C)(C)CCC7(C)C)C(C)(C)CCC5(C)C)ccc21 ; CC(C)(C)c1ccc2sc3c(N4c5cc6c(cc5B5c7cc8c(cc7N(c7ccc9c(c7)C(C)(C)CCC9(C)C)c7cc(N9c%10ccccc%10C%10(C)CCCCC9%10C)cc4c75)C(C)(C)CCC8(C)C)C(C)(C)CCC6(C)C)cccc3c2c1]

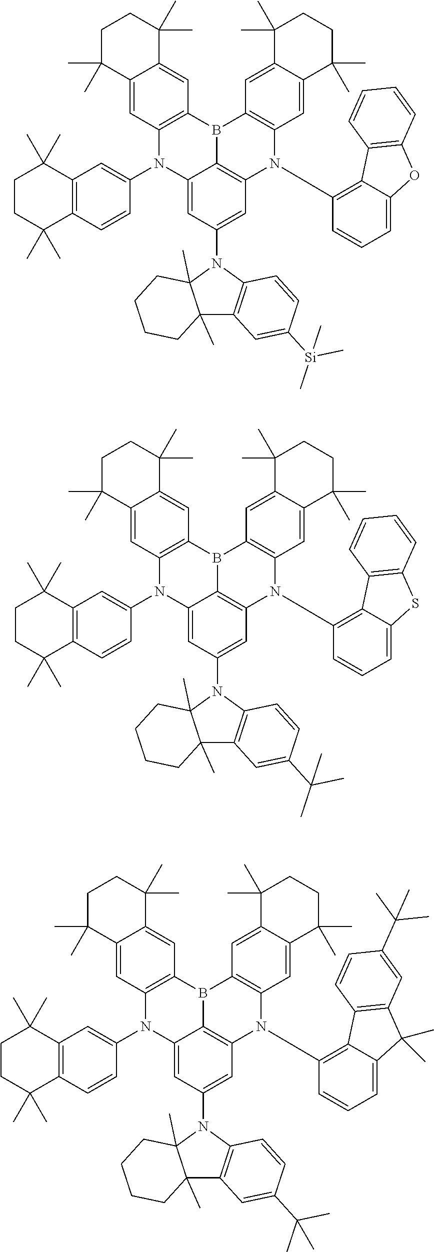 [SMILES: CC1(C)CCC(C)(C)c2cc(N3c4cc5c(cc4B4c6cc7c(cc6N(c6cccc8oc9ccccc9c68)c6cc(N8c9ccc([Si](C)(C)C)cc9C9(C)CCCCC89C)cc3c64)C(C)(C)CCC7(C)C)C(C)(C)CCC5(C)C)ccc21 ; CC(C)(C)c1ccc2c(c1)C1(C)CCCCC1(C)N2c1cc2c3c(c1)N(c1cccc4sc5ccccc5c14)c1cc4c(cc1B3c1cc3c(cc1N2c1ccc2c(c1)C(C)(C)CCC2(C)C)C(C)(C)CCC3(C)C)C(C)(C)CCC4(C)C ; CC(C)(C)c1ccc2c(c1)C(C)(C)c1cccc(N3c4cc5c(cc4B4c6cc7c(cc6N(c6ccc8c(c6)C(C)(C)CCC8(C)C)c6cc(N8c9ccc(C(C)(C)C)cc9C9(C)CCCCC89C)cc3c64)C(C)(C)CCC7(C)C)C(C)(C)CCC5(C)C)c1-2]

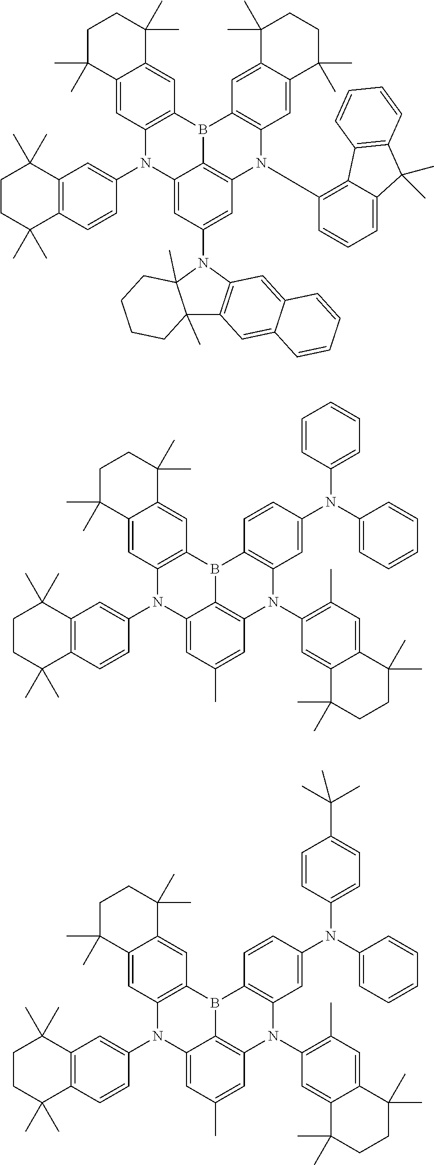

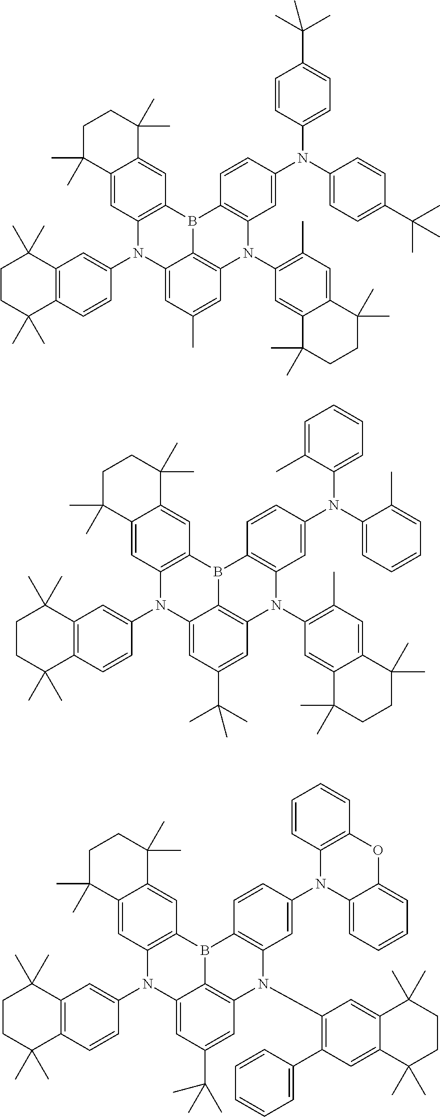

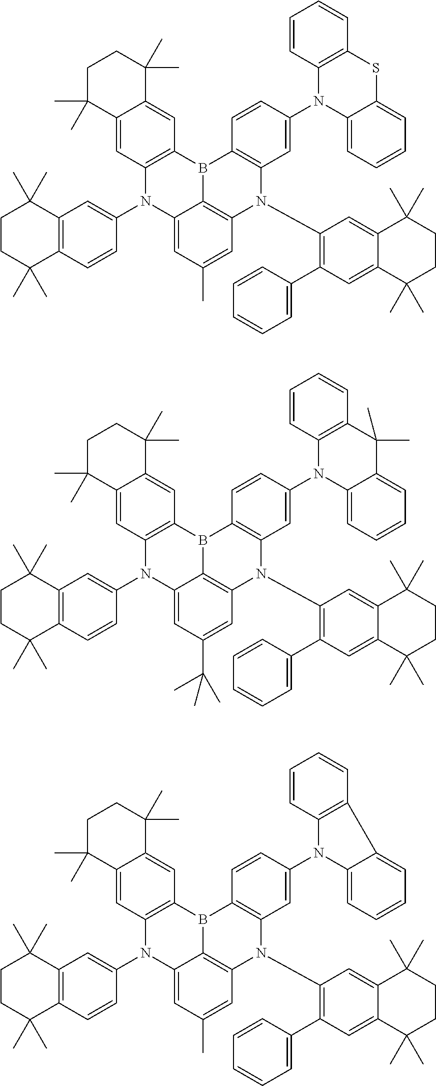 [SMILES: Cc1cc2c3c(c1)N(c1cc4c(cc1-c1ccccc1)C(C)(C)CCC4(C)C)c1cc(N4c5ccccc5Sc5ccccc54)ccc1B3c1cc3c(cc1N2c1ccc2c(c1)C(C)(C)CCC2(C)C)C(C)(C)CCC3(C)C ; CC(C)(C)c1cc2c3c(c1)N(c1cc4c(cc1-c1ccccc1)C(C)(C)CCC4(C)C)c1cc(N4c5ccccc5C(C)(C)c5ccccc54)ccc1B3c1cc3c(cc1N2c1ccc2c(c1)C(C)(C)CCC2(C)C)C(C)(C)CCC3(C)C ; Cc1cc2c3c(c1)N(c1cc4c(cc1-c1ccccc1)C(C)(C)CCC4(C)C)c1cc(-n4c5ccccc5c5ccccc54)ccc1B3c1cc3c(cc1N2c1ccc2c(c1)C(C)(C)CCC2(C)C)C(C)(C)CCC3(C)C]

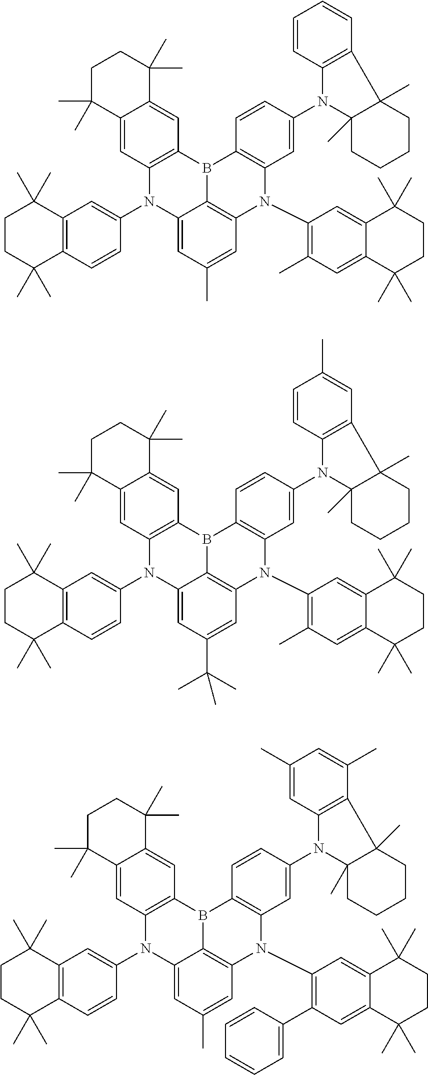 [SMILES: Cc1cc2c3c(c1)N(c1cc4c(cc1C)C(C)(C)CCC4(C)C)c1cc(N4c5ccccc5C5(C)CCCCC45C)ccc1B3c1cc3c(cc1N2c1ccc2c(c1)C(C)(C)CCC2(C)C)C(C)(C)CCC3(C)C ; Cc1ccc2c(c1)C1(C)CCCCC1(C)N2c1ccc2c(c1)N(c1cc3c(cc1C)C(C)(C)CCC3(C)C)c1cc(C(C)(C)C)cc3c1B2c1cc2c(cc1N3c1ccc3c(c1)C(C)(C)CCC3(C)C)C(C)(C)CCC2(C)C ; Cc1cc2c3c(c1)N(c1cc4c(cc1-c1ccccc1)C(C)(C)CCC4(C)C)c1cc(N4c5cc(C)cc(C)c5C5(C)CCCCC45C)ccc1B3c1cc3c(cc1N2c1ccc2c(c1)C(C)(C)CCC2(C)C)C(C)(C)CCC3(C)C]

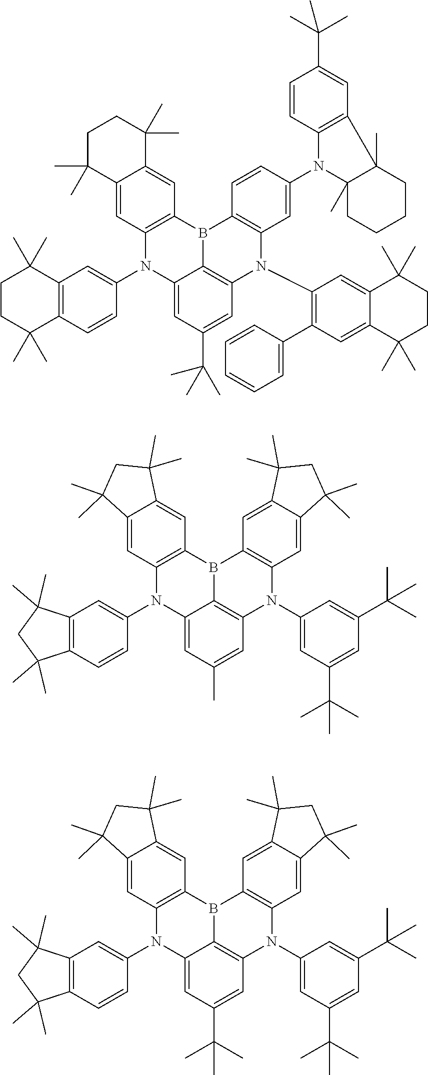

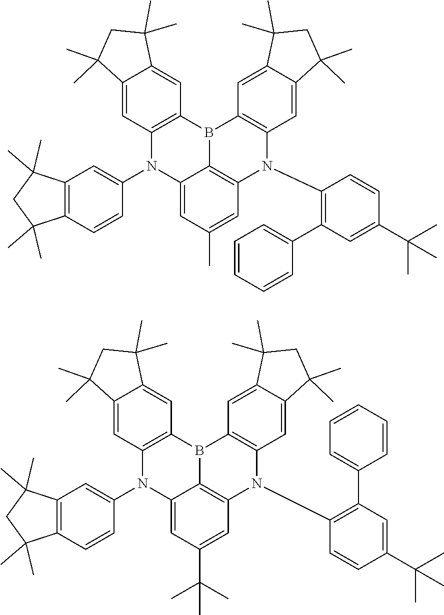

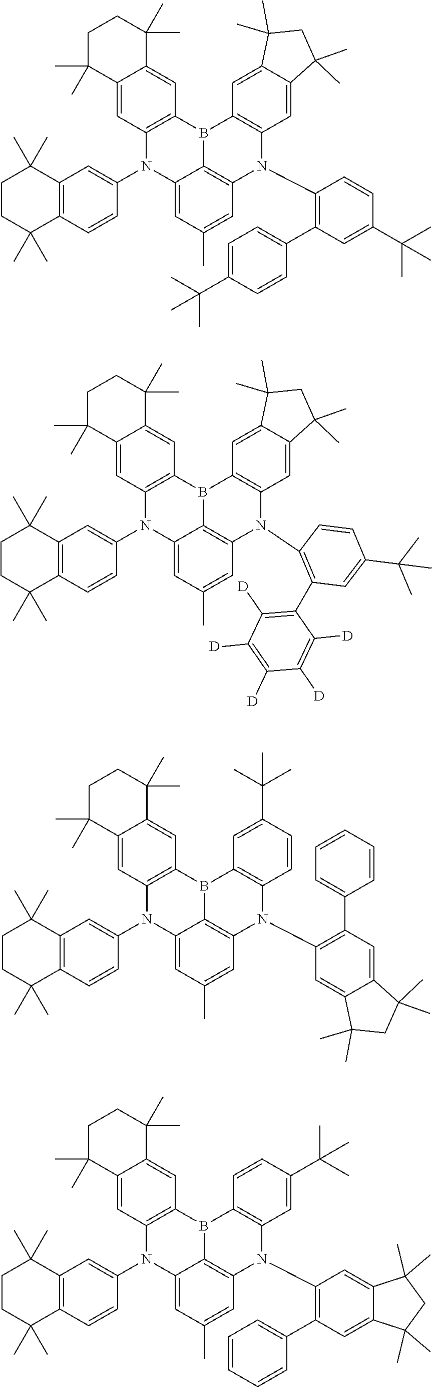

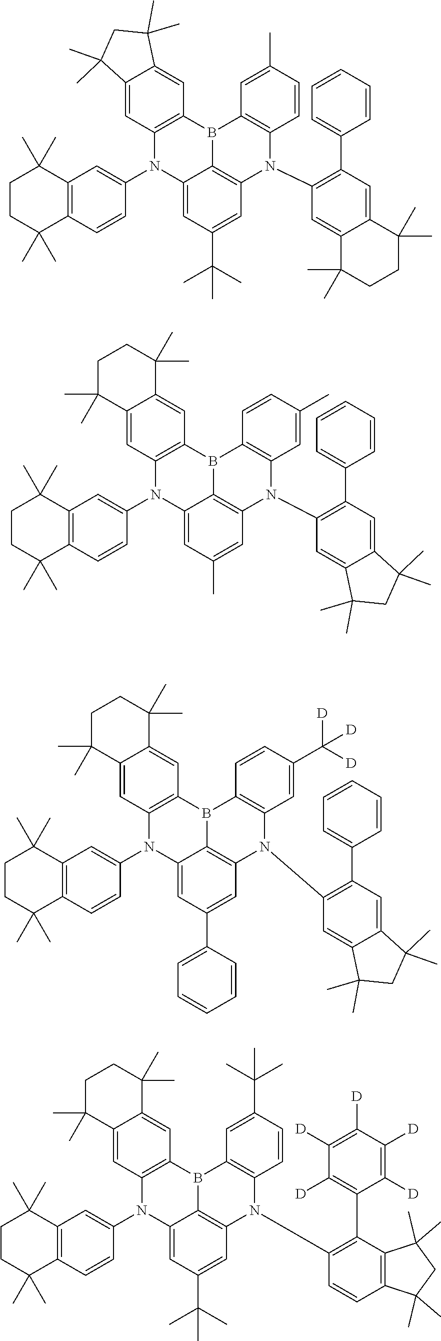

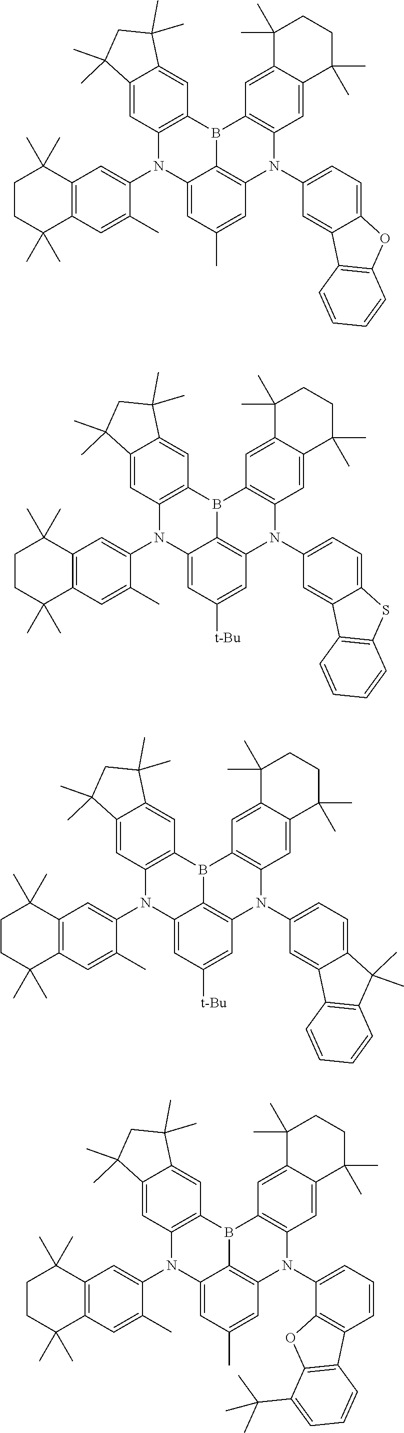

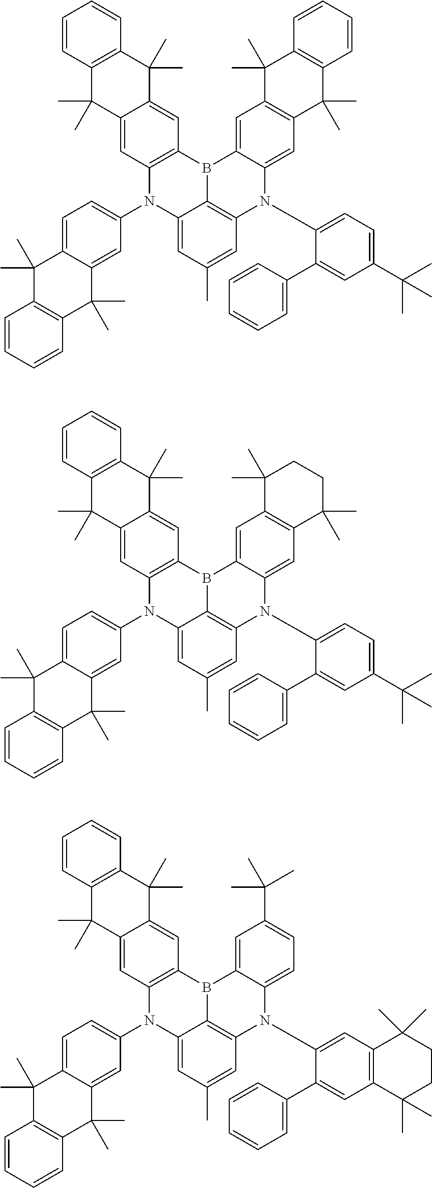

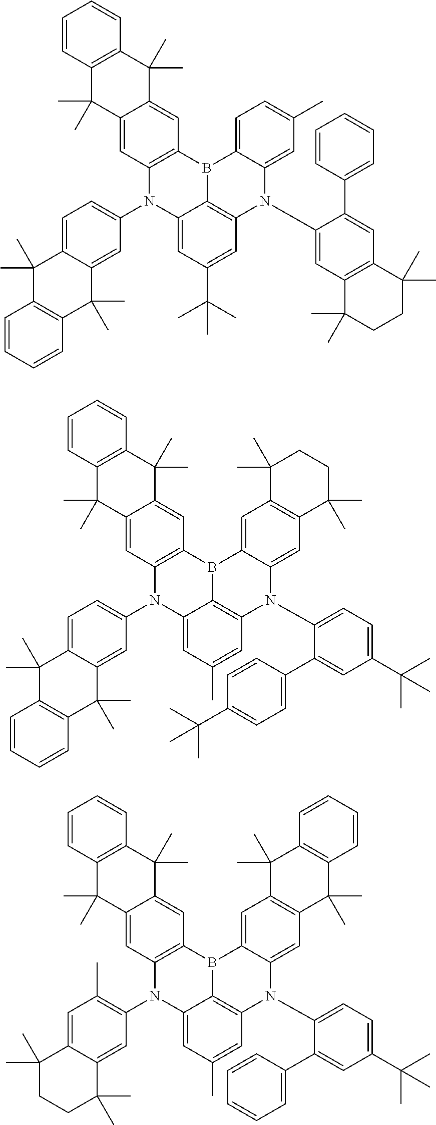

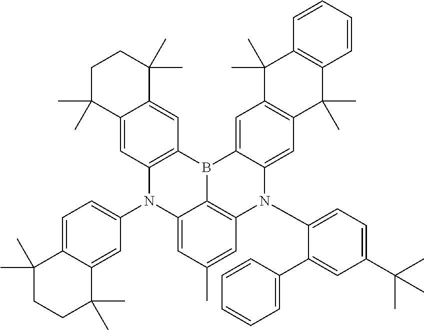 [SMILES: Cc1cc2c3c(c1)N(c1ccc(C(C)(C)C)cc1-c1ccccc1)c1cc4c(cc1B3c1cc3c(cc1N2c1ccc2c(c1)C(C)(C)CCC2(C)C)C(C)(C)CCC3(C)C)C(C)(C)c1ccccc1C4(C)C]

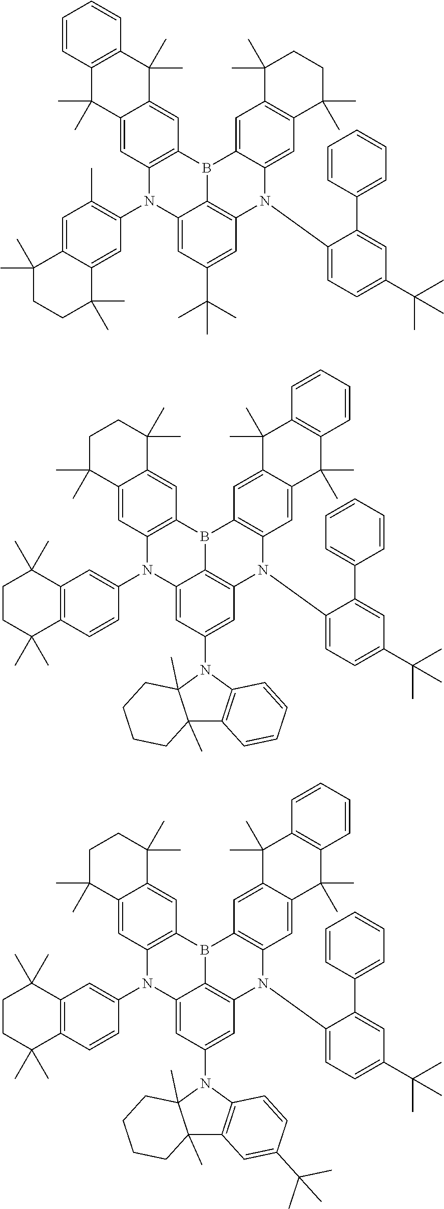 [SMILES: Cc1cc2c(cc1N1c3cc4c(cc3B3c5cc6c(cc5N(c5ccc(C(C)(C)C)cc5-c5ccccc5)c5cc(C(C)(C)C)cc1c53)C(C)(C)CCC6(C)C)C(C)(C)c1ccccc1C4(C)C)C(C)(C)CCC2(C)C ; CC(C)(C)c1ccc(N2c3cc4c(cc3B3c5cc6c(cc5N(c5ccc7c(c5)C(C)(C)CCC7(C)C)c5cc(N7c8ccccc8C8(C)CCCCC78C)cc2c53)C(C)(C)CCC6(C)C)C(C)(C)c2ccccc2C4(C)C)c(-c2ccccc2)c1 ; CC(C)(C)c1ccc(N2c3cc4c(cc3B3c5cc6c(cc5N(c5ccc7c(c5)C(C)(C)CCC7(C)C)c5cc(N7c8ccc(C(C)(C)C)cc8C8(C)CCCCC78C)cc2c53)C(C)(C)CCC6(C)C)C(C)(C)c2ccccc2C4(C)C)c(-c2ccccc2)c1]

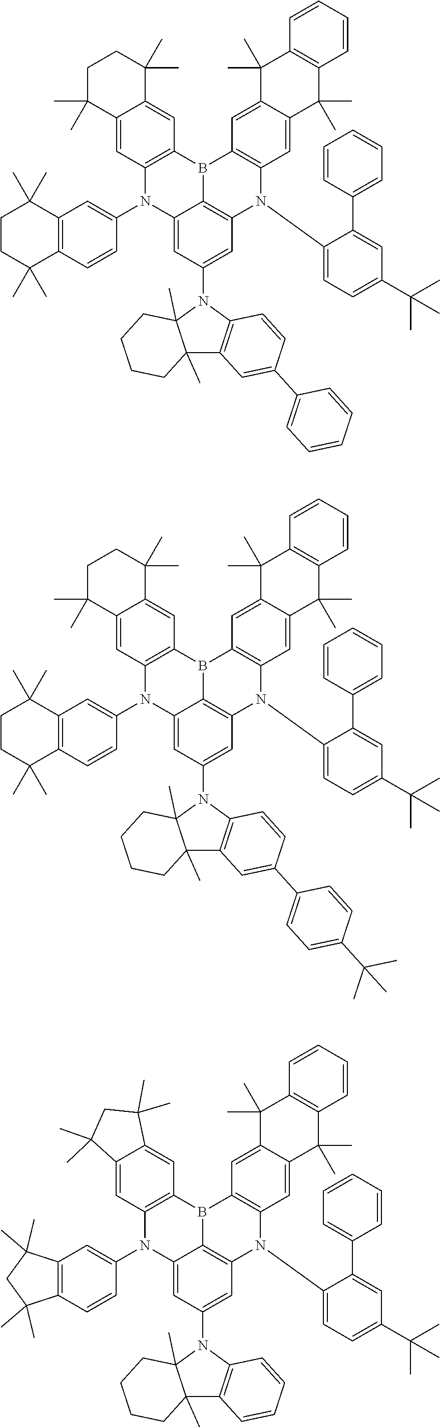

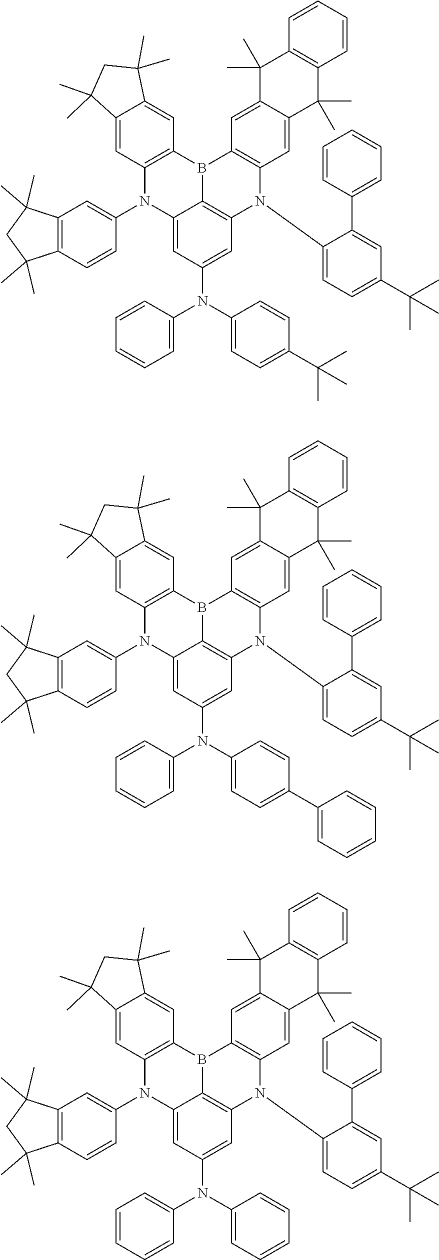

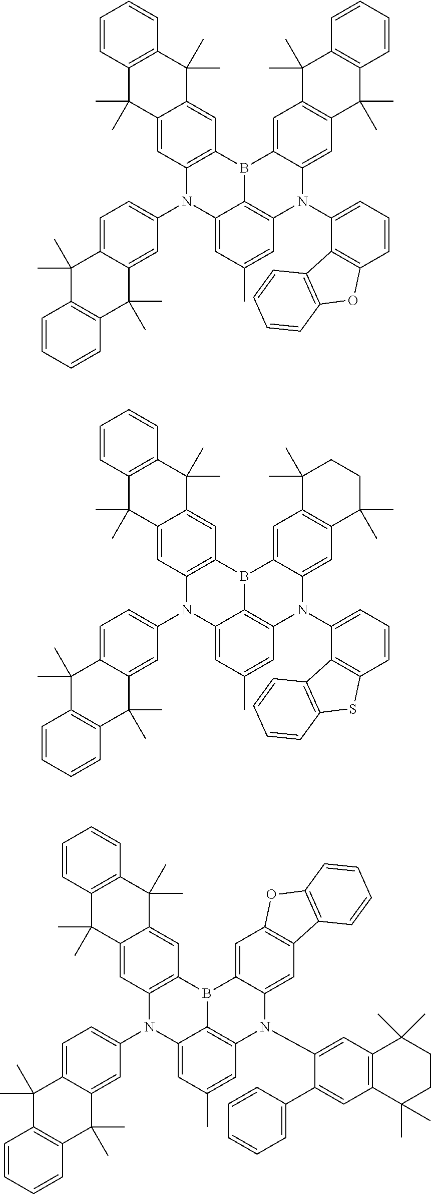

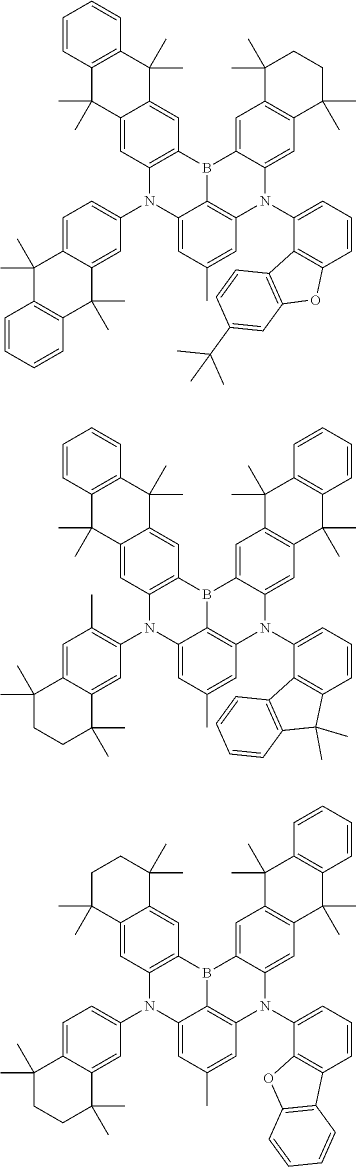

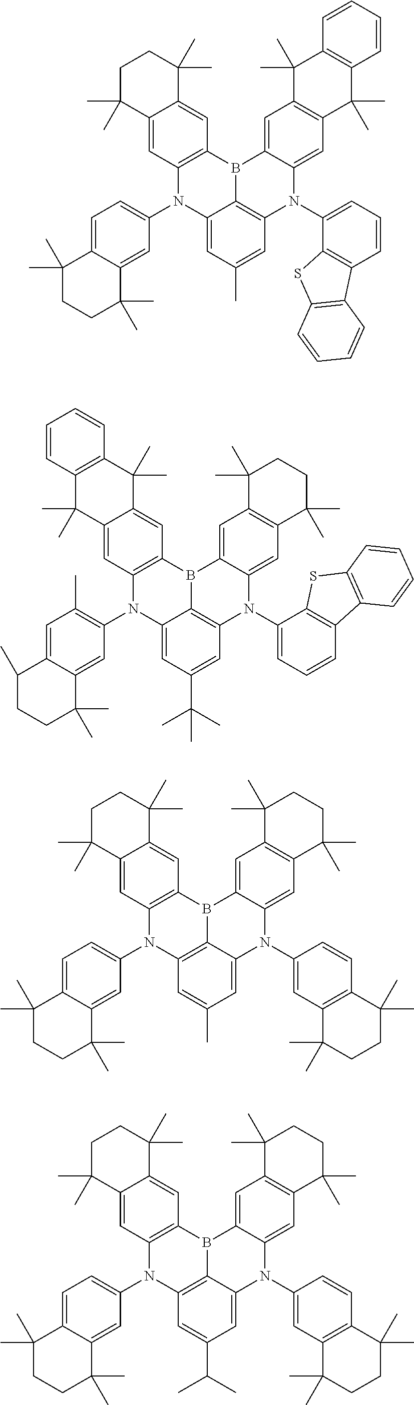

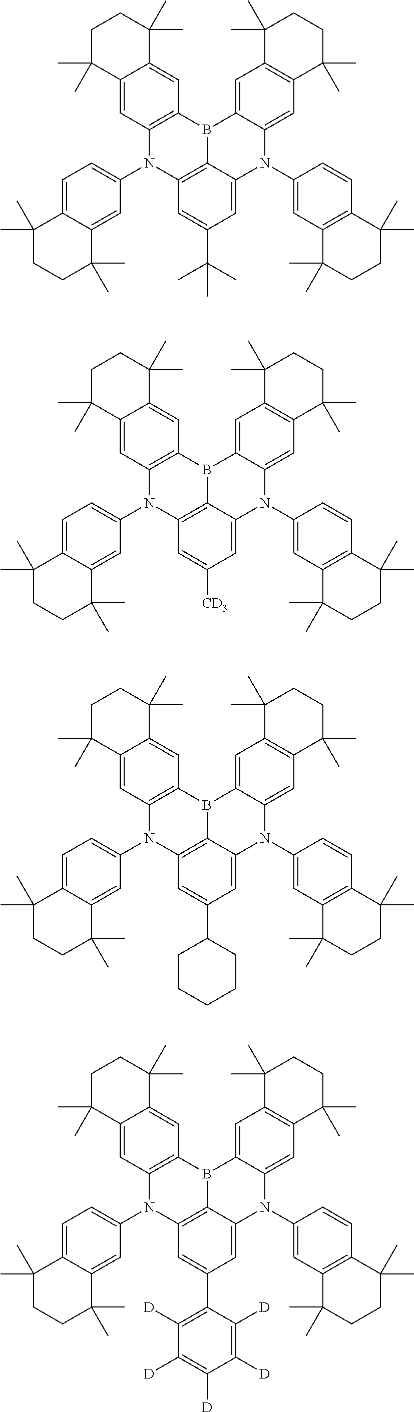

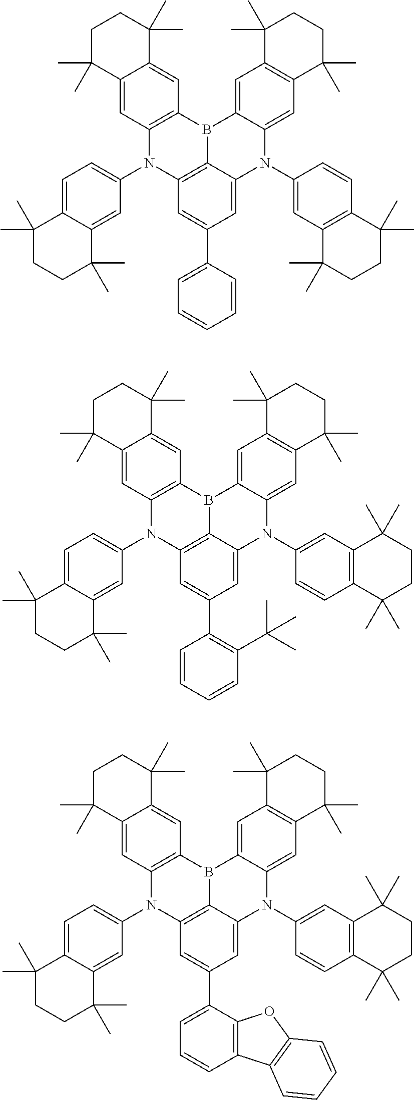

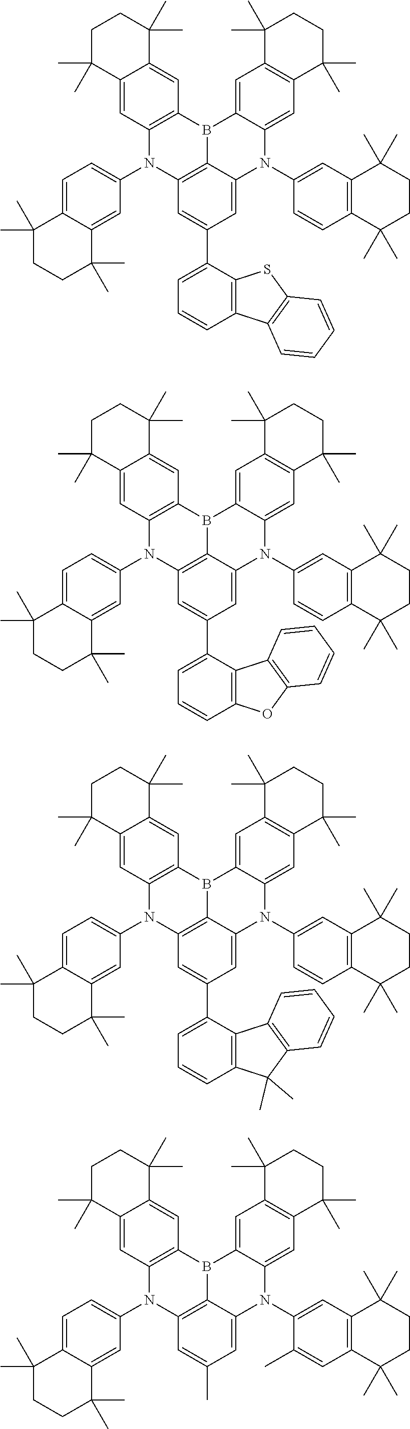

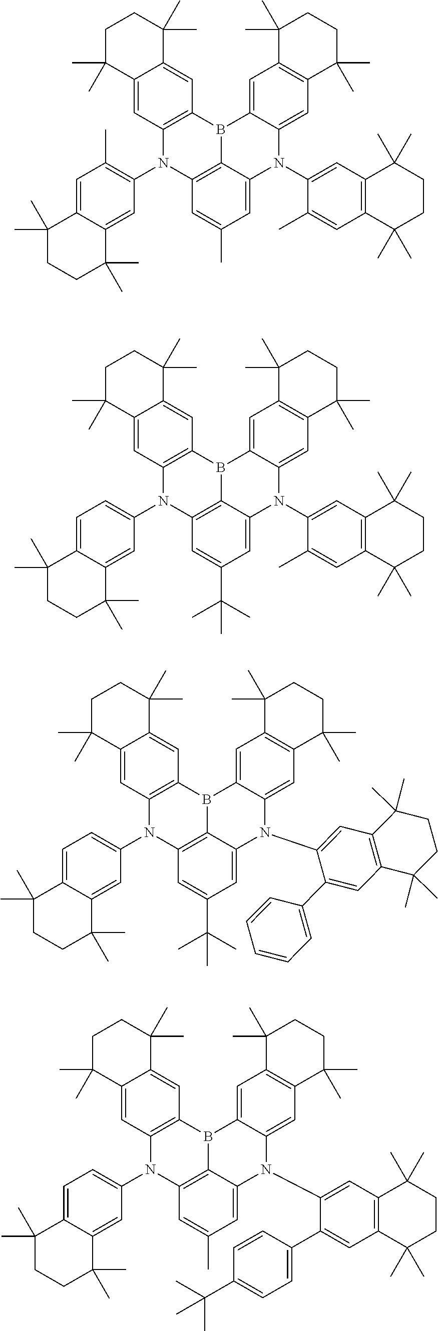

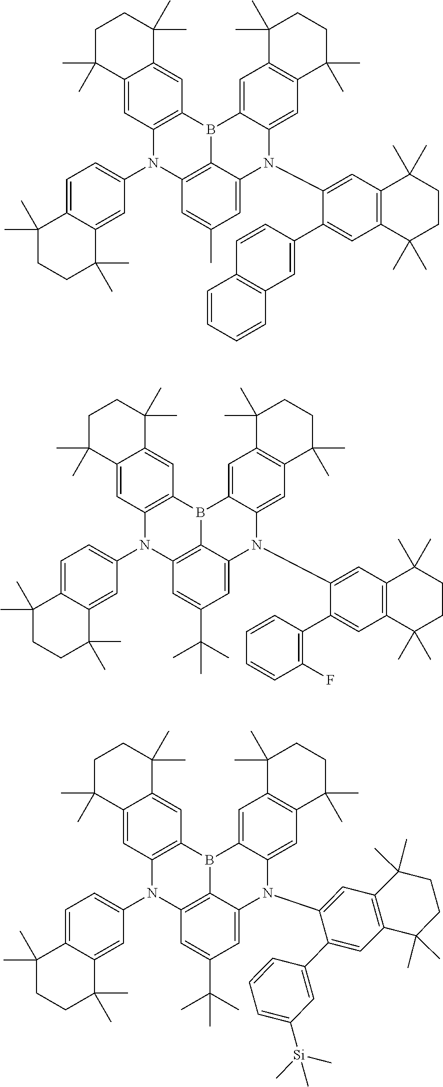

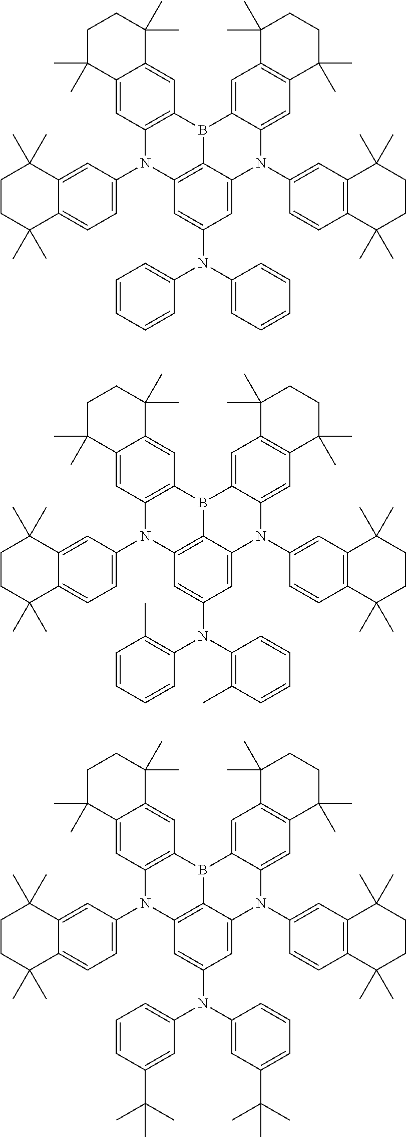

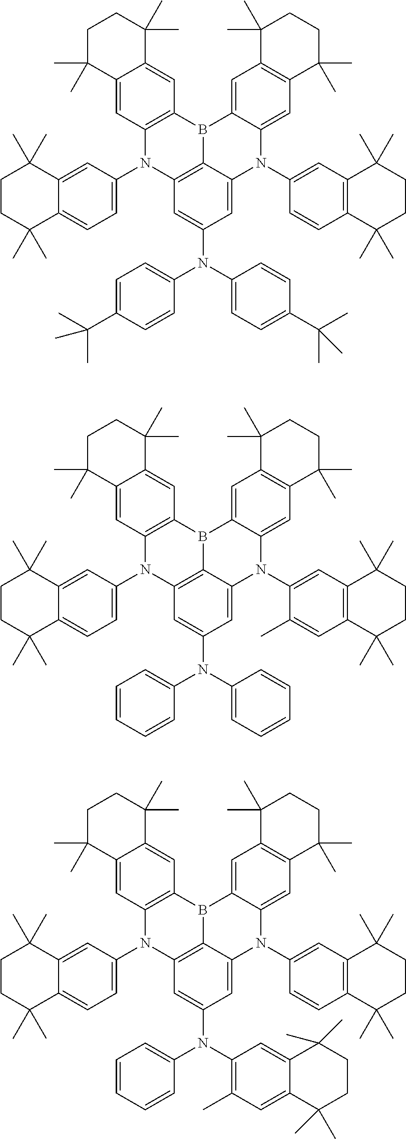 [SMILES: CC(C)(C)c1ccc(N(c2ccc(C(C)(C)C)cc2)c2cc3c4c(c2)N(c2ccc5c(c2)C(C)(C)CCC5(C)C)c2cc5c(cc2B4c2cc4c(cc2N3c2ccc3c(c2)C(C)(C)CCC3(C)C)C(C)(C)CCC4(C)C)C(C)(C)CCC5(C)C)cc1 ; Cc1cc2c(cc1N1c3cc4c(cc3B3c5cc6c(cc5N(c5ccc7c(c5)C(C)(C)CCC7(C)C)c5cc(N(c7ccccc7)c7ccccc7)cc1c53)C(C)(C)CCC6(C)C)C(C)(C)CCC4(C)C)C(C)(C)CCC2(C)C ; Cc1cc2c(cc1N(c1ccccc1)c1cc3c4c(c1)N(c1ccc5c(c1)C(C)(C)CCC5(C)C)c1cc5c(cc1B4c1cc4c(cc1N3c1ccc3c(c1)C(C)(C)CCC3(C)C)C(C)(C)CCC4(C)C)C(C)(C)CCC5(C)C)C(C)(C)CCC2(C)C]

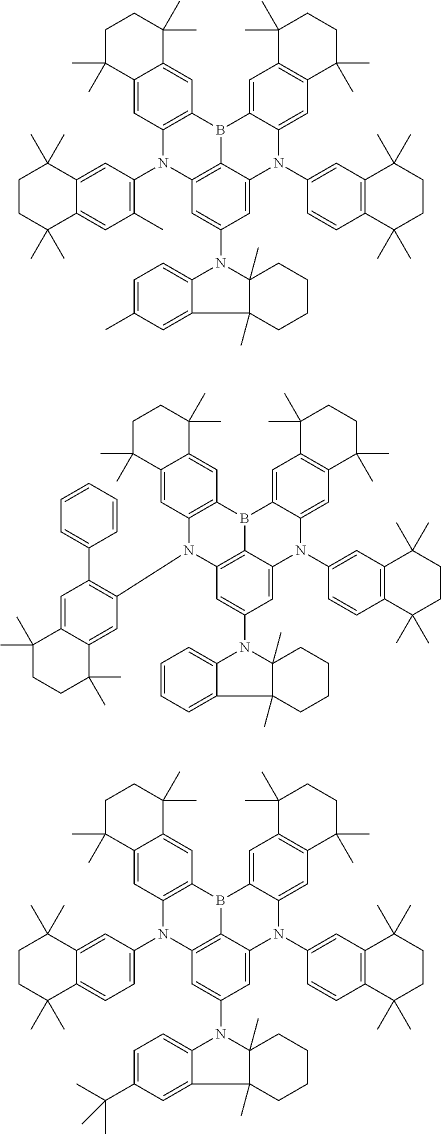

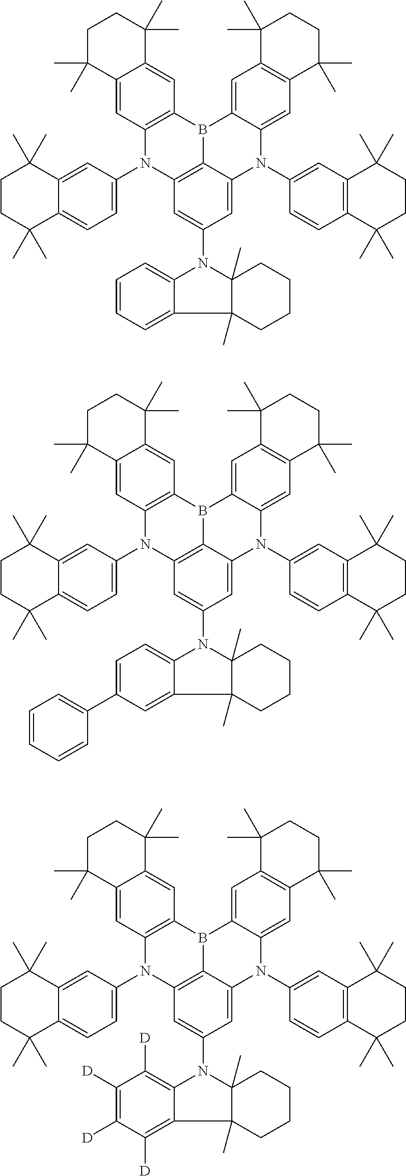

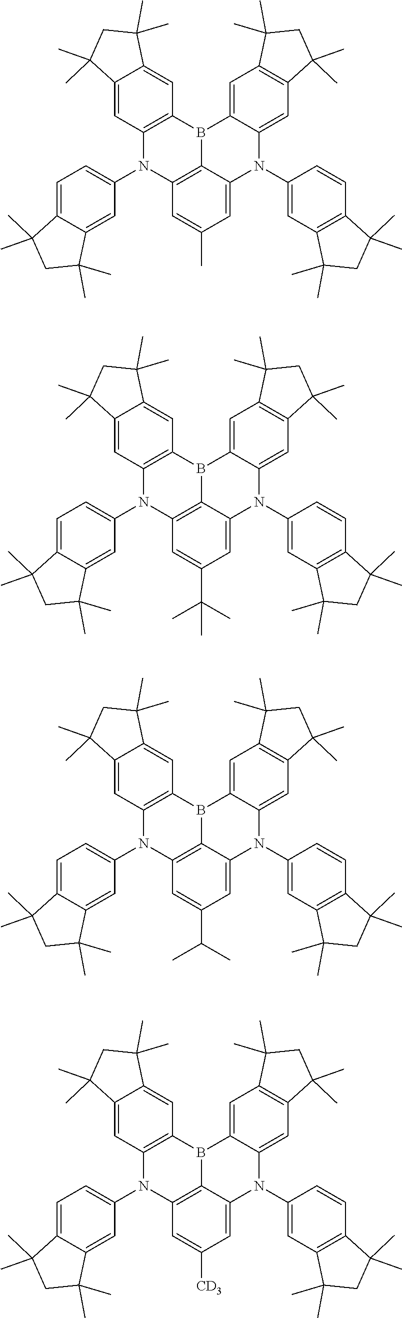

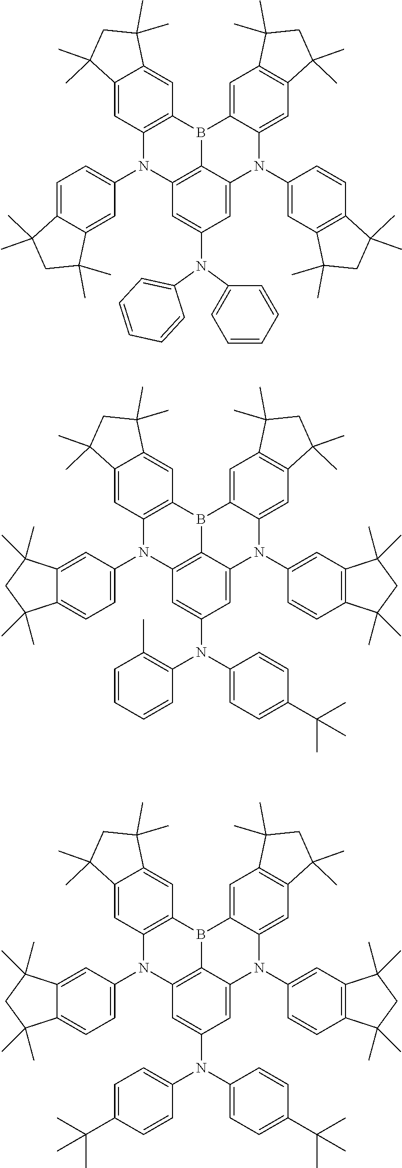

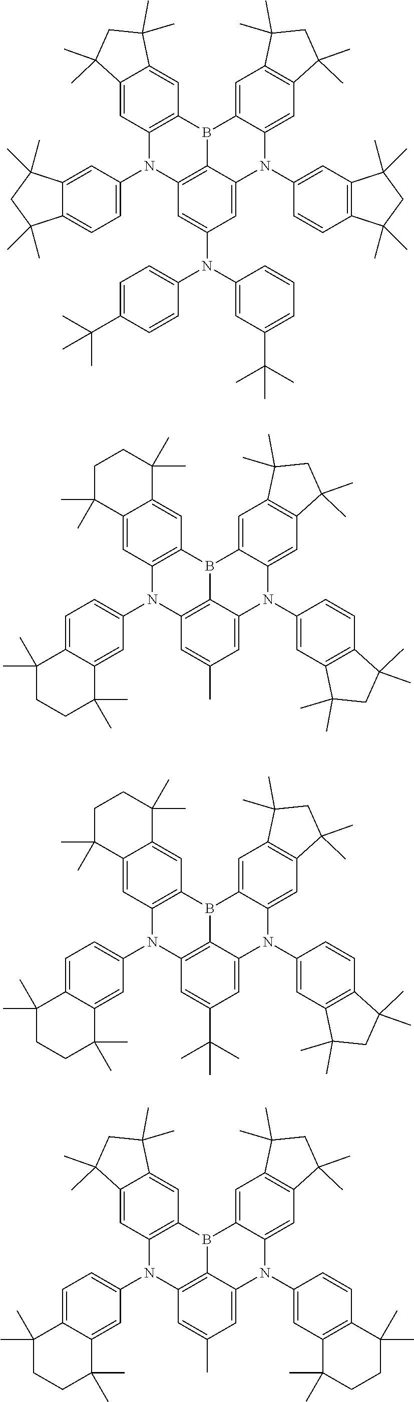

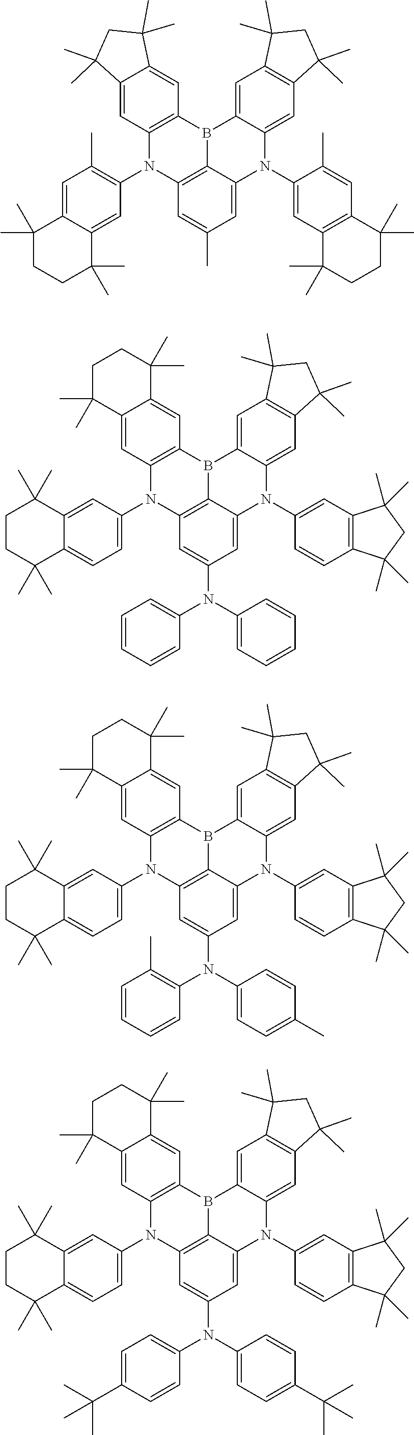

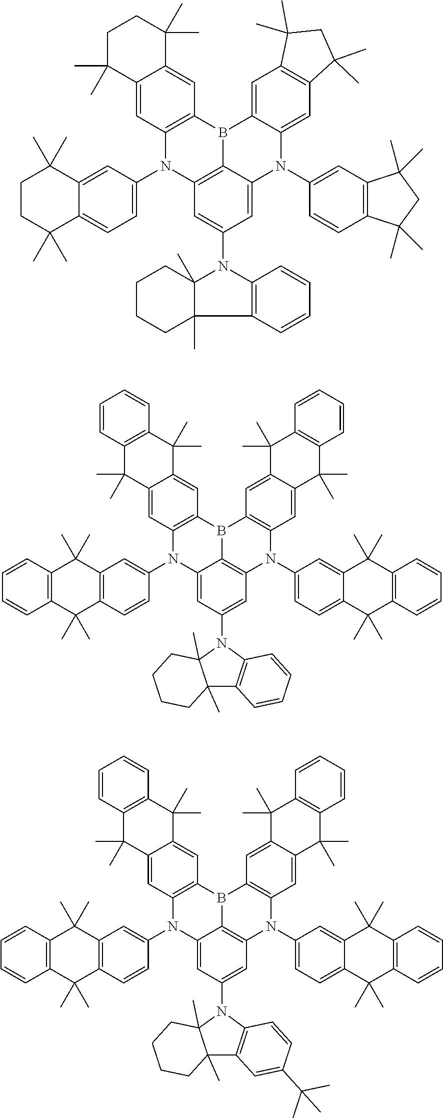

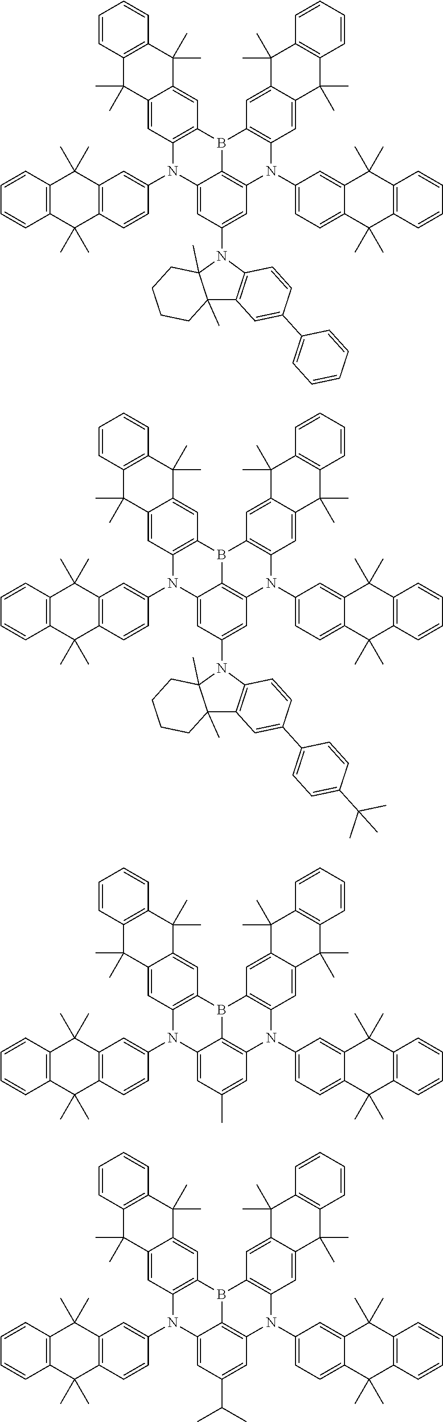

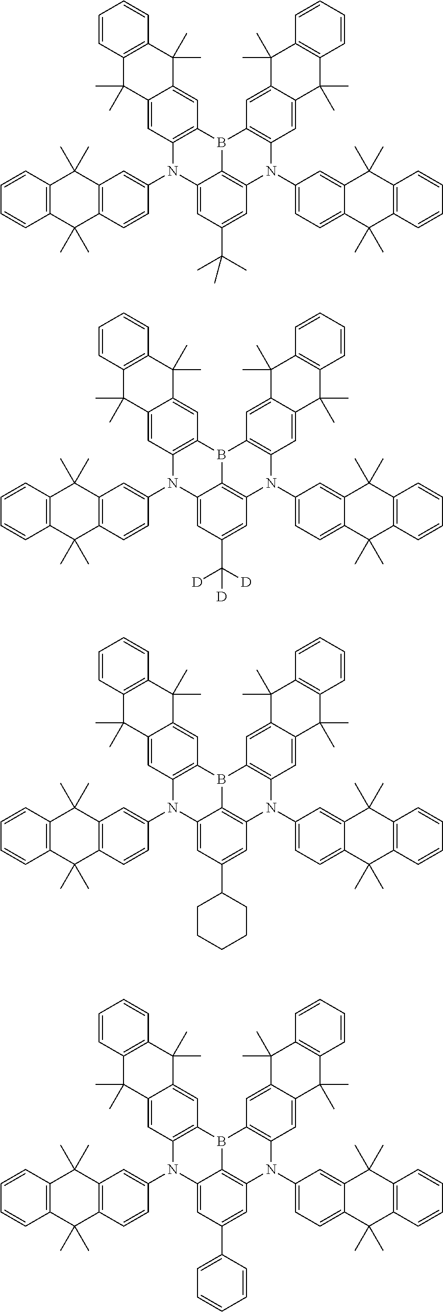

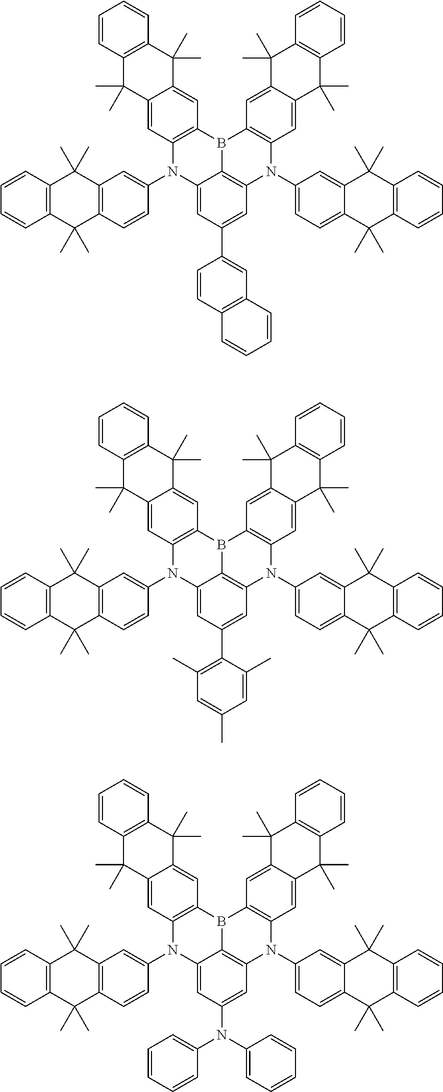 [SMILES: CC1(C)c2ccccc2C(C)(C)c2cc(N3c4cc5c(cc4B4c6cc7c(cc6N(c6ccc8c(c6)C(C)(C)c6ccccc6C8(C)C)c6cc(-c8ccc9ccccc9c8)cc3c64)C(C)(C)c3ccccc3C7(C)C)C(C)(C)c3ccccc3C5(C)C)ccc21 ; Cc1cc(C)c(-c2cc3c4c(c2)N(c2ccc5c(c2)C(C)(C)c2ccccc2C5(C)C)c2cc5c(cc2B4c2cc4c(cc2N3c2ccc3c(c2)C(C)(C)c2ccccc2C3(C)C)C(C)(C)c2ccccc2C4(C)C)C(C)(C)c2ccccc2C5(C)C)c(C)c1 ; CC1(C)c2ccccc2C(C)(C)c2cc(N3c4cc5c(cc4B4c6cc7c(cc6N(c6ccc8c(c6)C(C)(C)c6ccccc6C8(C)C)c6cc(N(c8ccccc8)c8ccccc8)cc3c64)C(C)(C)c3ccccc3C7(C)C)C(C)(C)c3ccccc3C5(C)C)ccc21]

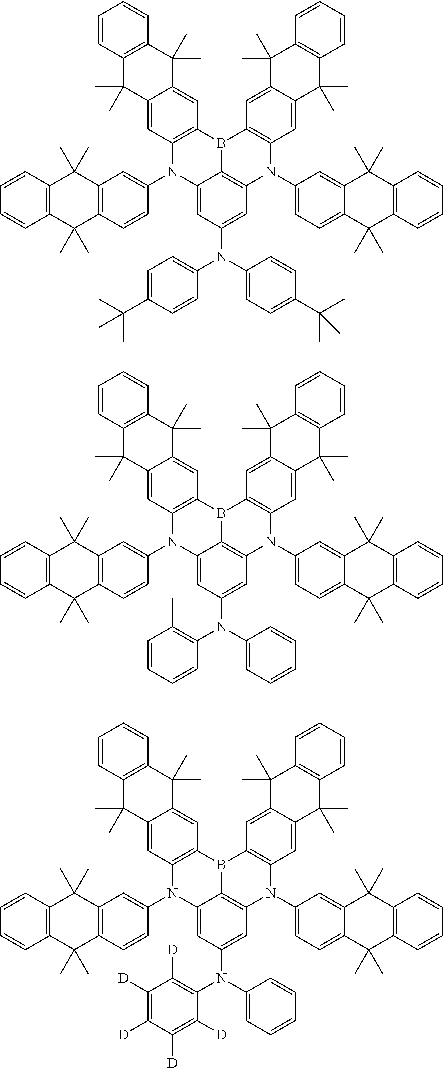

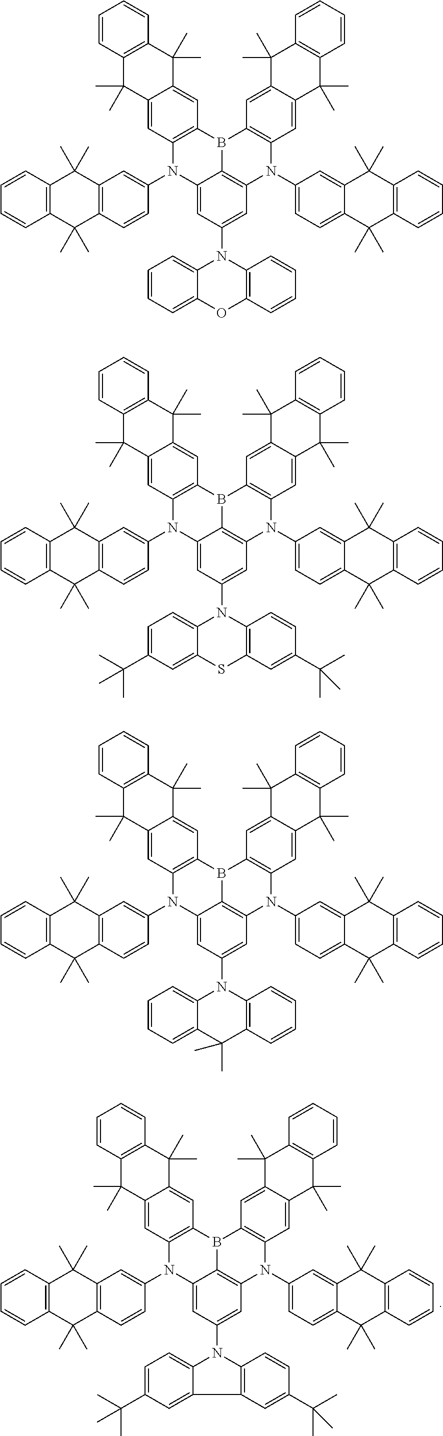

9. The organic light emitting device of claim 1, wherein the compound of Chemical Formula 2 is one compound selected from among the following compounds:

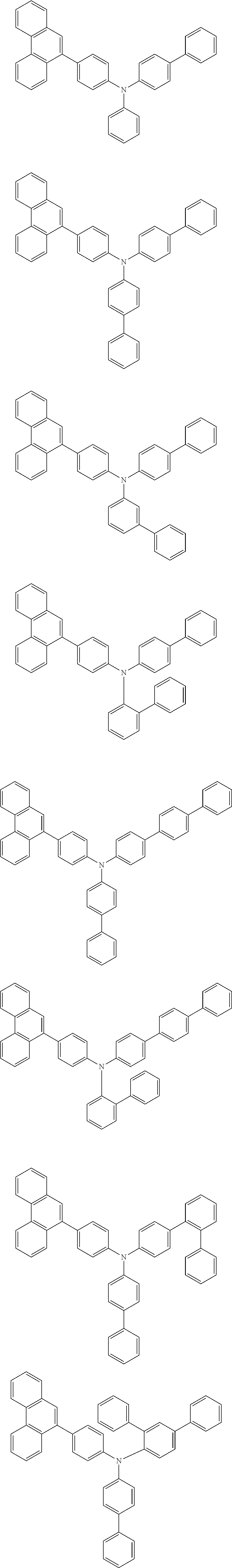

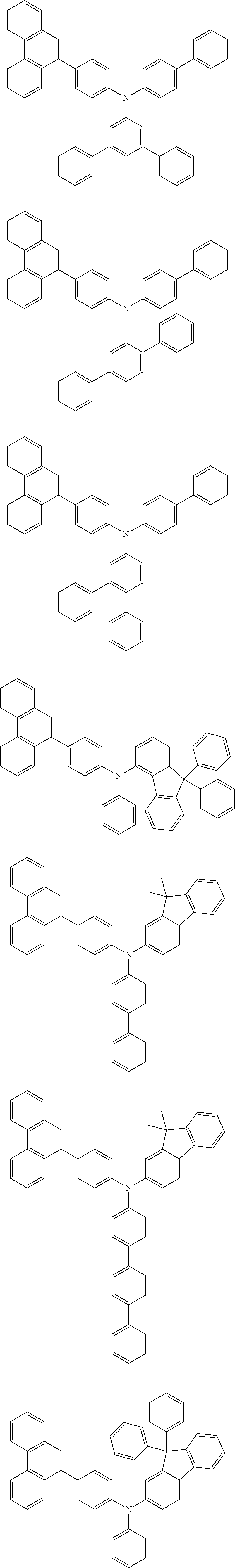

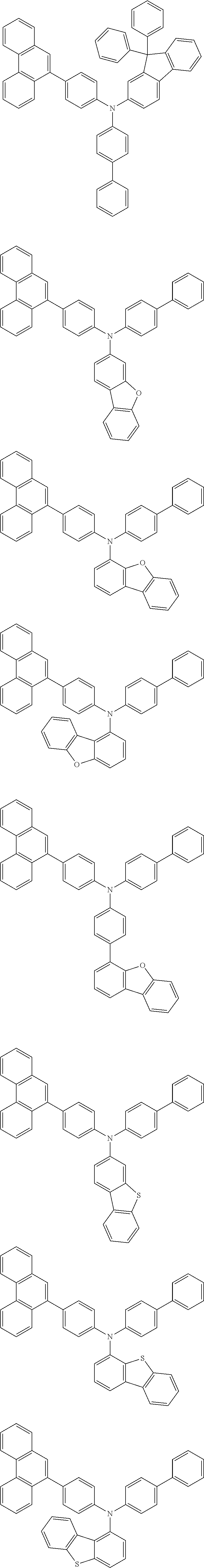

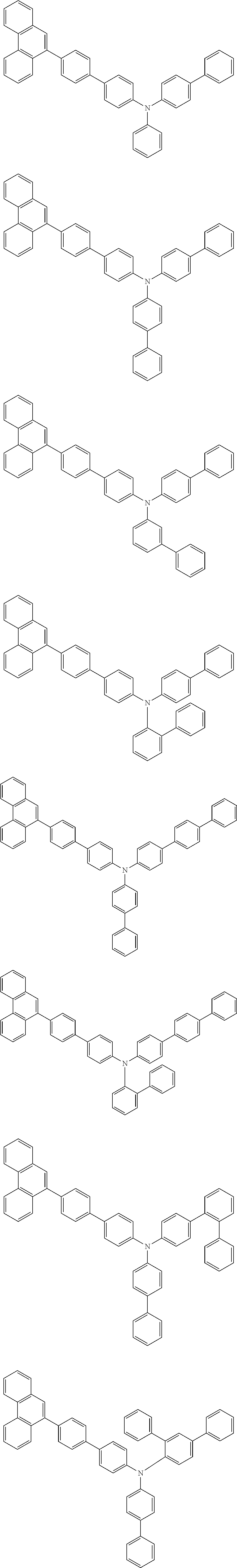

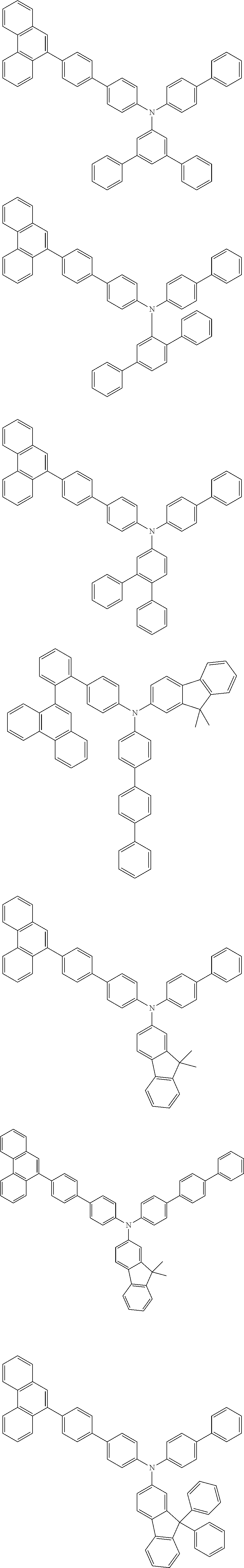

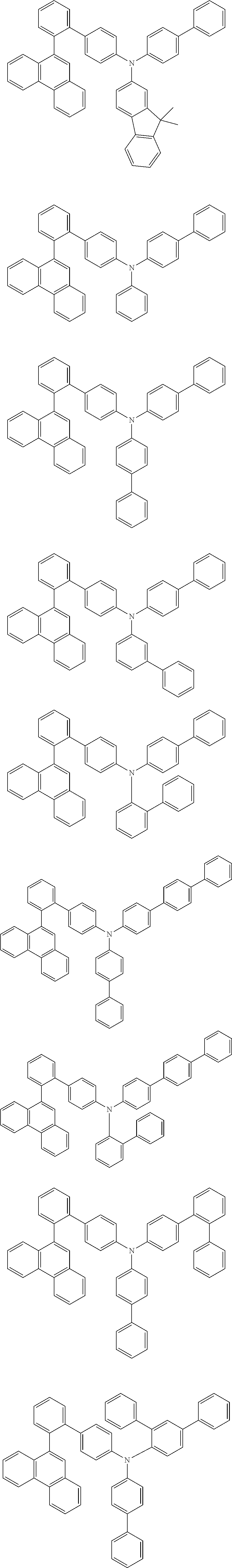

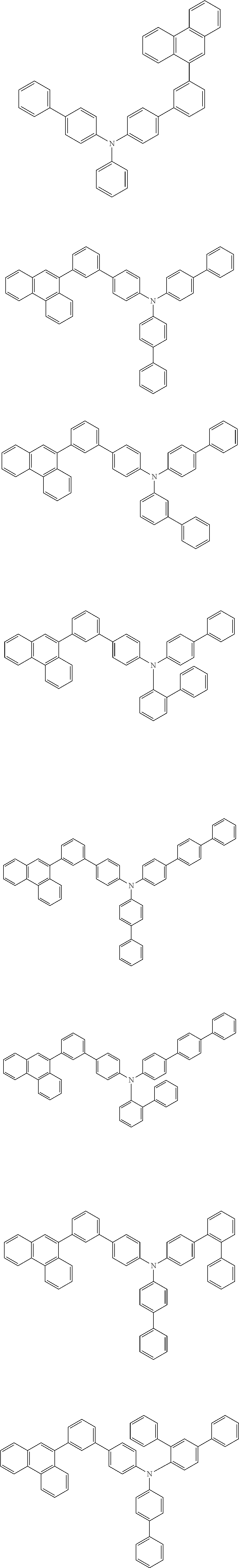

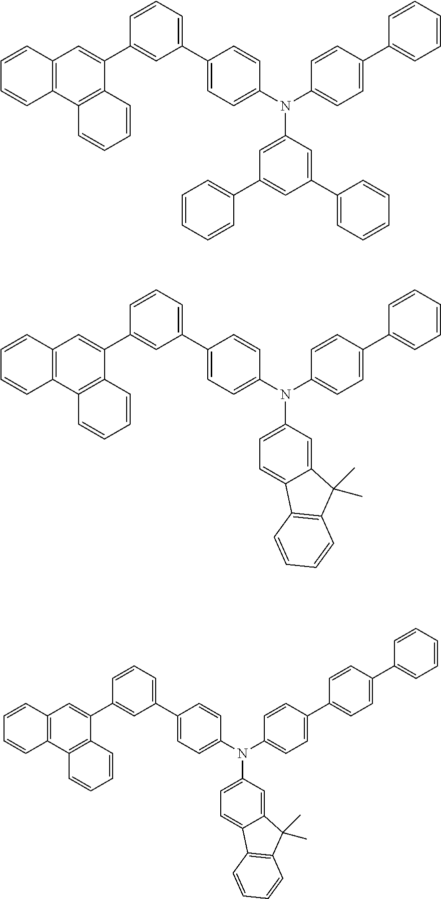 [SMILES: c1ccc(-c2ccc(N(c3ccc(-c4cccc(-c5cc6ccccc6c6ccccc56)c4)cc3)c3cc(-c4ccccc4)cc(-c4ccccc4)c3)cc2)cc1 ; CC1(C)c2ccccc2-c2ccc(N(c3ccc(-c4ccccc4)cc3)c3ccc(-c4cccc(-c5cc6ccccc6c6ccccc56)c4)cc3)cc21 ; CC1(C)c2ccccc2-c2ccc(N(c3ccc(-c4ccc(-c5ccccc5)cc4)cc3)c3ccc(-c4cccc(-c5cc6ccccc6c6ccccc56)c4)cc3)cc21]

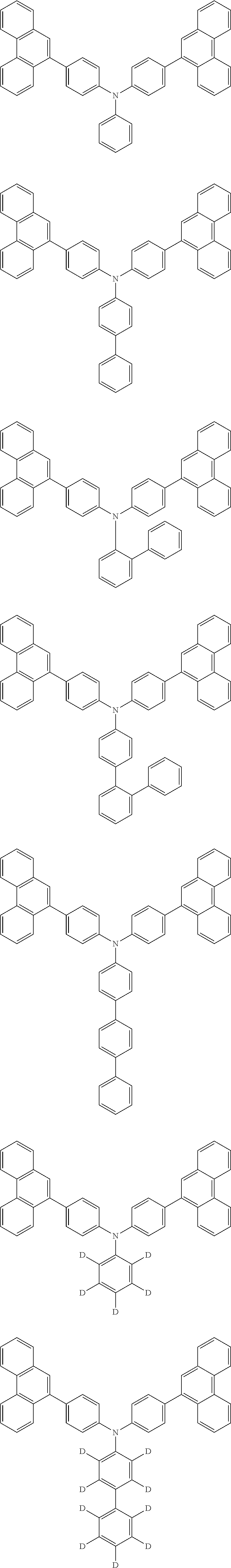

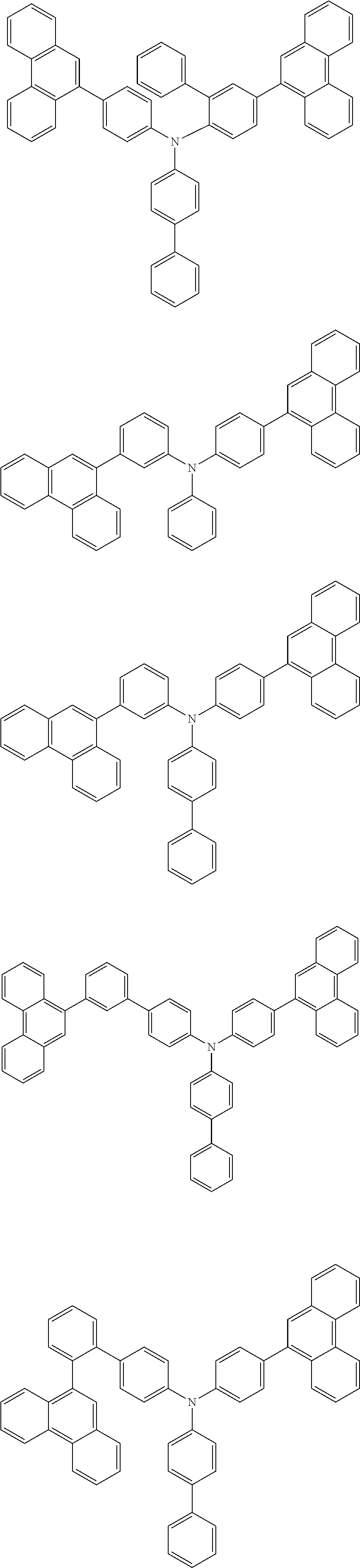 [SMILES: c1ccc(-c2ccc(N(c3ccc(-c4cc5ccccc5c5ccccc45)cc3)c3ccc(-c4cc5ccccc5c5ccccc45)cc3-c3ccccc3)cc2)cc1 ; c1ccc(N(c2ccc(-c3cc4ccccc4c4ccccc34)cc2)c2cccc(-c3cc4ccccc4c4ccccc34)c2)cc1 ; c1ccc(-c2ccc(N(c3ccc(-c4cc5ccccc5c5ccccc45)cc3)c3cccc(-c4cc5ccccc5c5ccccc45)c3)cc2)cc1 ; c1ccc(-c2ccc(N(c3ccc(-c4cccc(-c5cc6ccccc6c6ccccc56)c4)cc3)c3ccc(-c4cc5ccccc5c5ccccc45)cc3)cc2)cc1 ; c1ccc(-c2ccc(N(c3ccc(-c4ccccc4-c4cc5ccccc5c5ccccc45)cc3)c3ccc(-c4cc5ccccc5c5ccccc45)cc3)cc2)cc1]

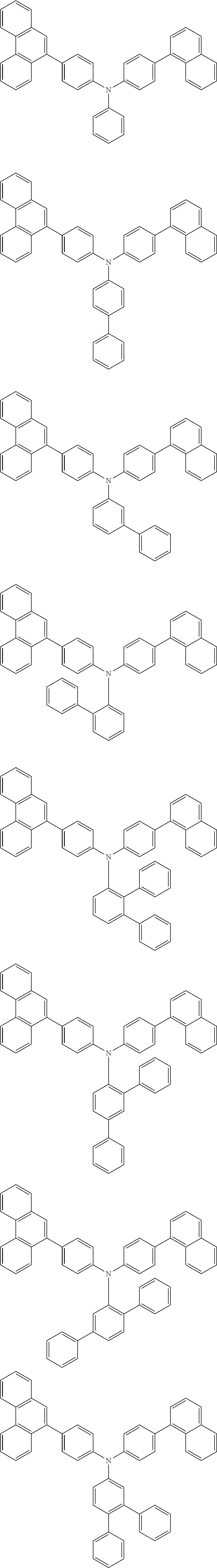

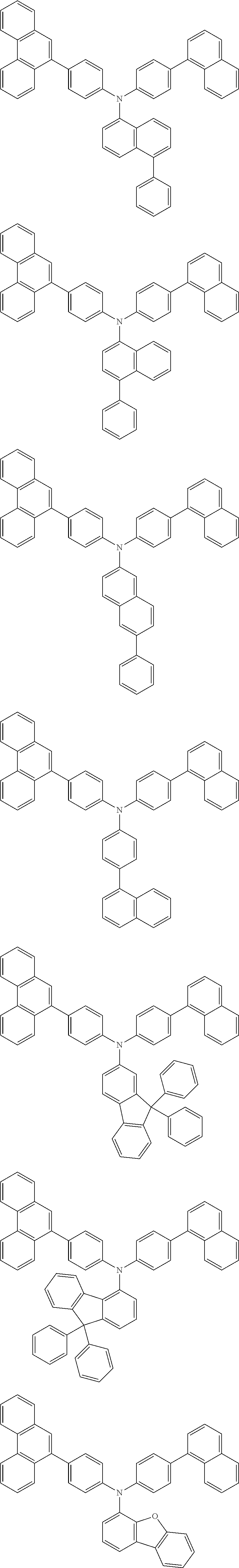

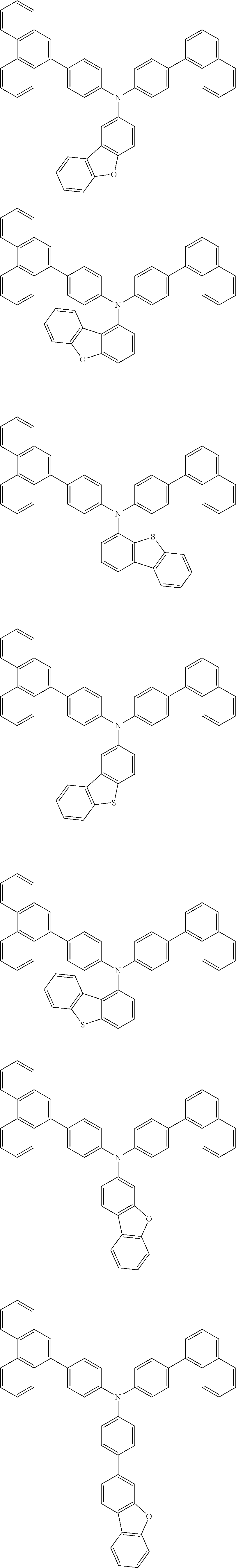

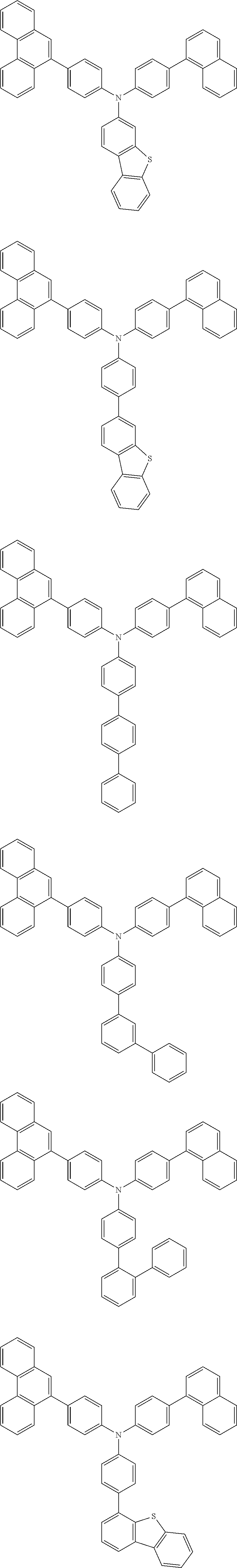

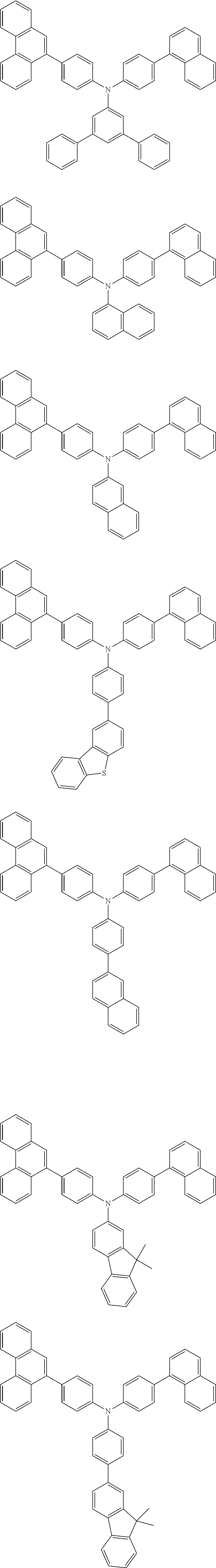

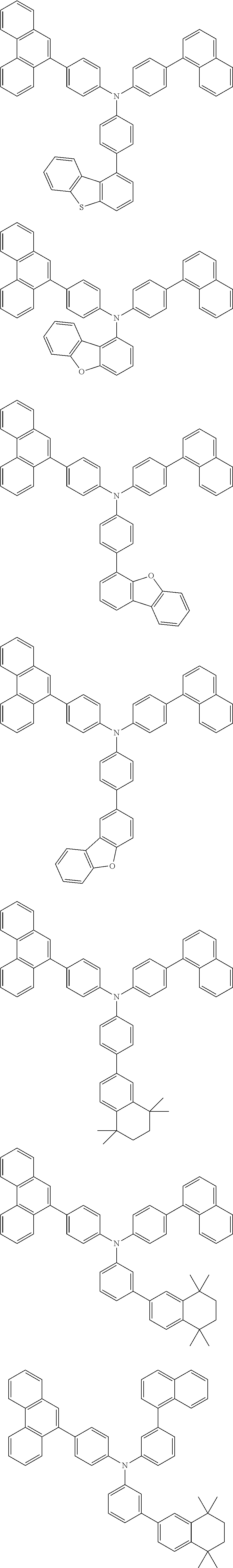

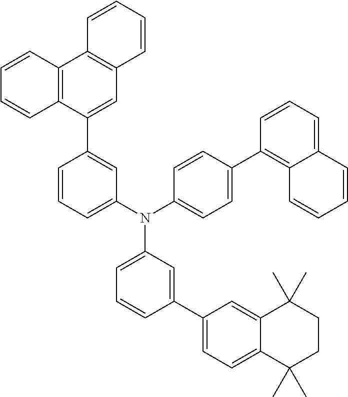 [SMILES: CC1(C)CCC(C)(C)c2cc(-c3cccc(N(c4ccc(-c5cccc6ccccc56)cc4)c4cccc(-c5cc6ccccc6c6ccccc56)c4)c3)ccc21]

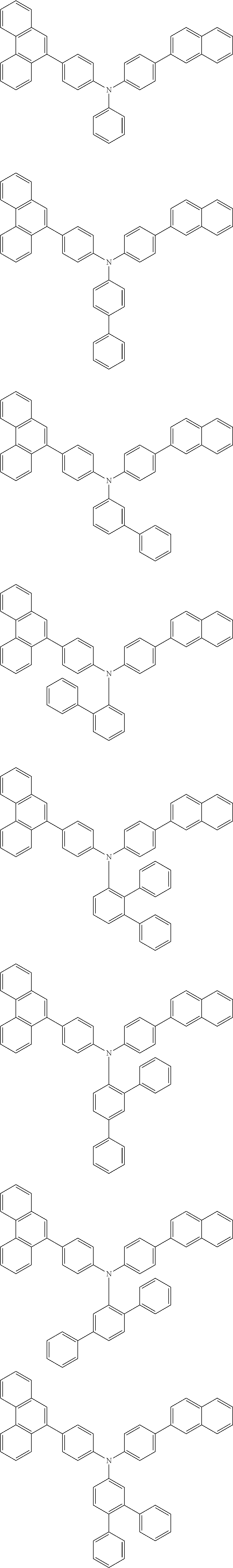

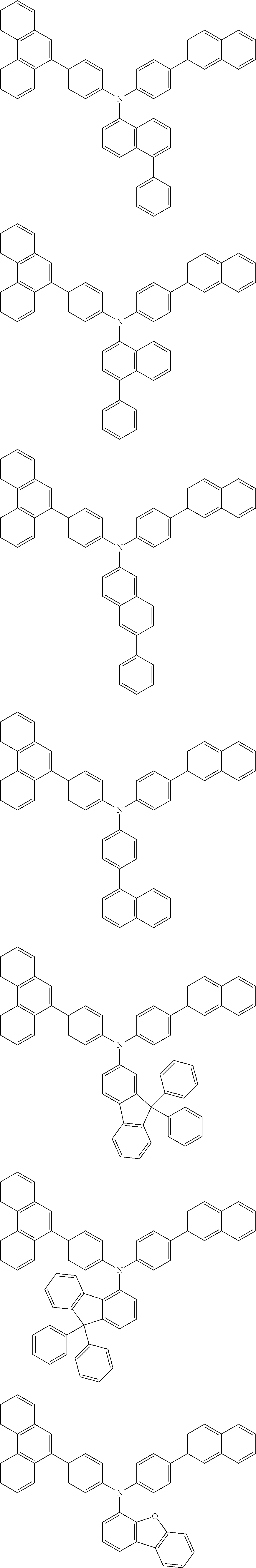

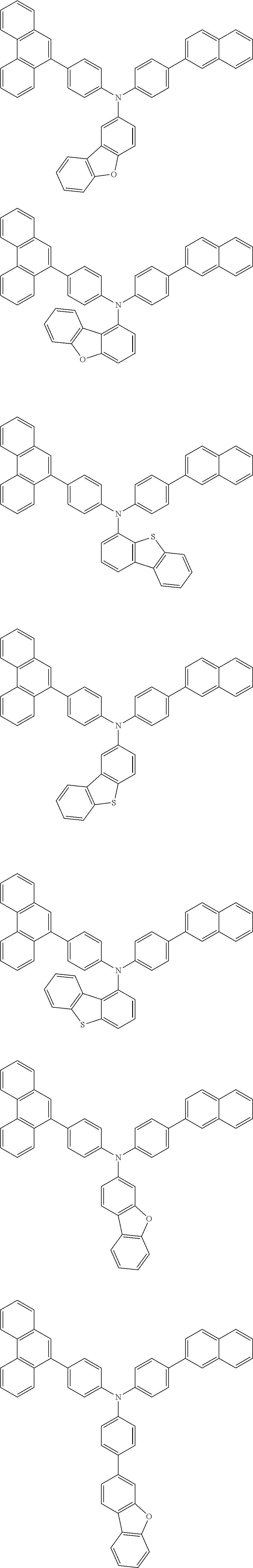

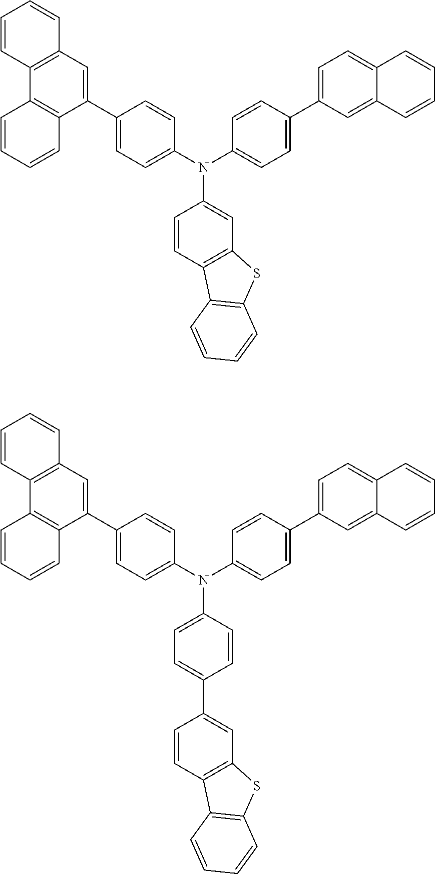 [SMILES: c1ccc2cc(-c3ccc(N(c4ccc(-c5cc6ccccc6c6ccccc56)cc4)c4ccc5c(c4)sc4ccccc45)cc3)ccc2c1 ; c1ccc2cc(-c3ccc(N(c4ccc(-c5ccc6c(c5)sc5ccccc56)cc4)c4ccc(-c5cc6ccccc6c6ccccc56)cc4)cc3)ccc2c1]

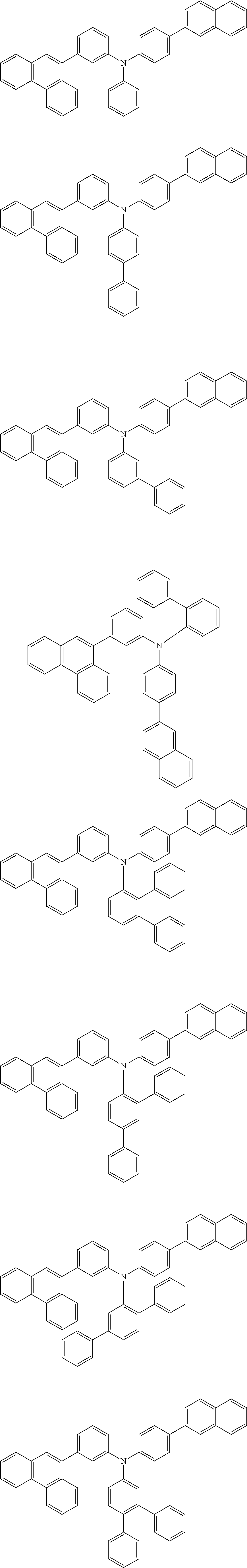

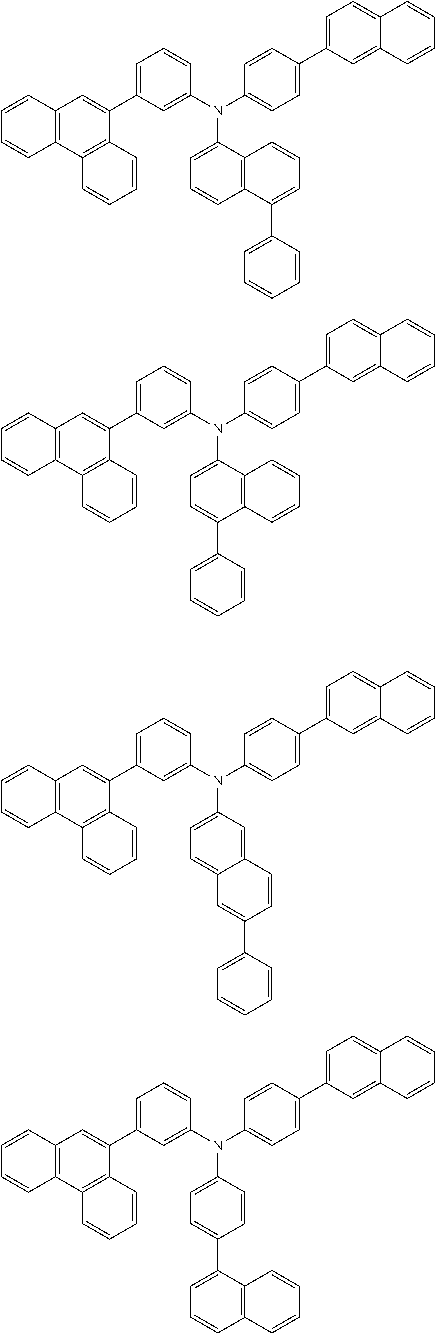 [SMILES: c1ccc(-c2cccc3c(N(c4ccc(-c5ccc6ccccc6c5)cc4)c4cccc(-c5cc6ccccc6c6ccccc56)c4)cccc23)cc1 ; c1ccc(-c2ccc(N(c3ccc(-c4ccc5ccccc5c4)cc3)c3cccc(-c4cc5ccccc5c5ccccc45)c3)c3ccccc23)cc1 ; c1ccc(-c2ccc3cc(N(c4ccc(-c5ccc6ccccc6c5)cc4)c4cccc(-c5cc6ccccc6c6ccccc56)c4)ccc3c2)cc1 ; c1cc(-c2cc3ccccc3c3ccccc23)cc(N(c2ccc(-c3ccc4ccccc4c3)cc2)c2ccc(-c3cccc4ccccc34)cc2)c1]

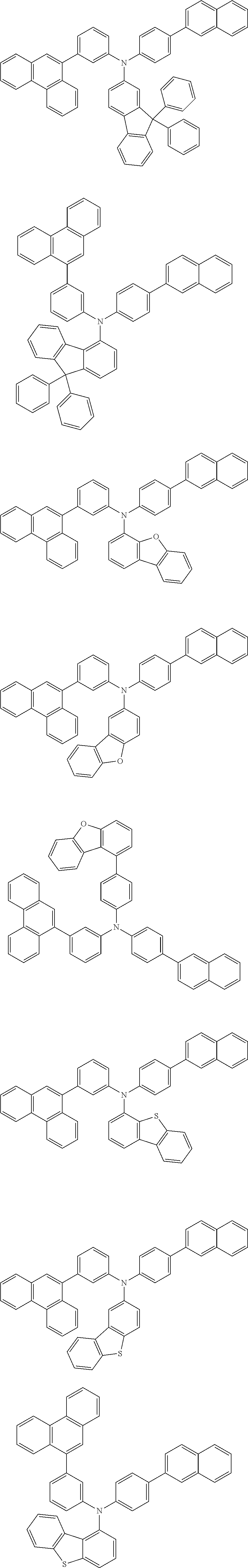

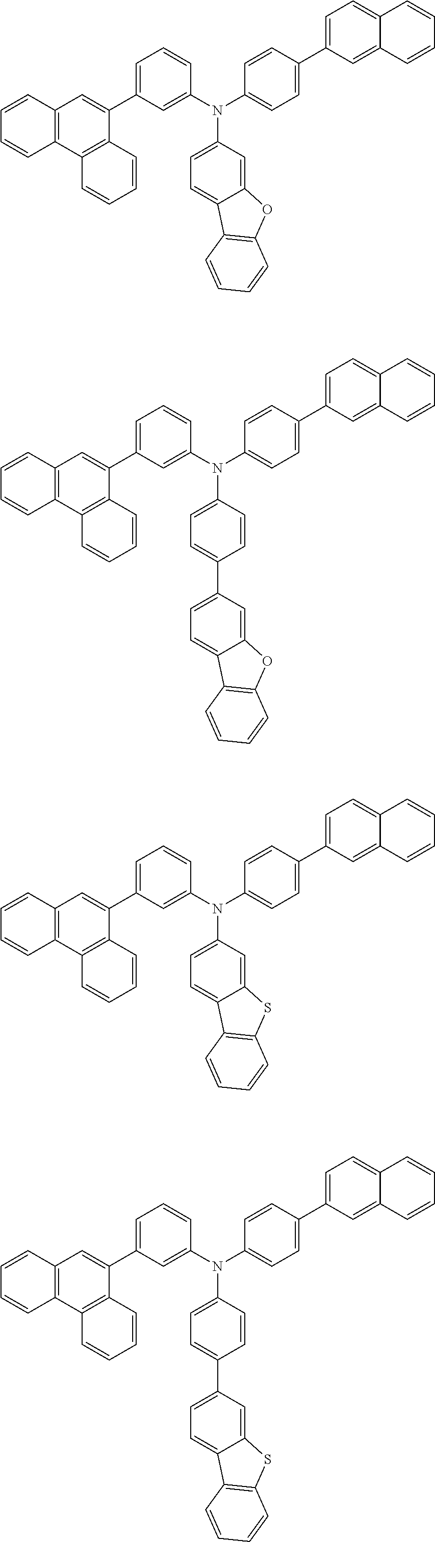 [SMILES: c1cc(-c2cc3ccccc3c3ccccc23)cc(N(c2ccc(-c3ccc4ccccc4c3)cc2)c2ccc3c(c2)oc2ccccc23)c1 ; c1cc(-c2cc3ccccc3c3ccccc23)cc(N(c2ccc(-c3ccc4ccccc4c3)cc2)c2ccc(-c3ccc4c(c3)oc3ccccc34)cc2)c1 ; c1cc(-c2cc3ccccc3c3ccccc23)cc(N(c2ccc(-c3ccc4ccccc4c3)cc2)c2ccc3c(c2)sc2ccccc23)c1 ; c1cc(-c2cc3ccccc3c3ccccc23)cc(N(c2ccc(-c3ccc4ccccc4c3)cc2)c2ccc(-c3ccc4c(c3)sc3ccccc34)cc2)c1]

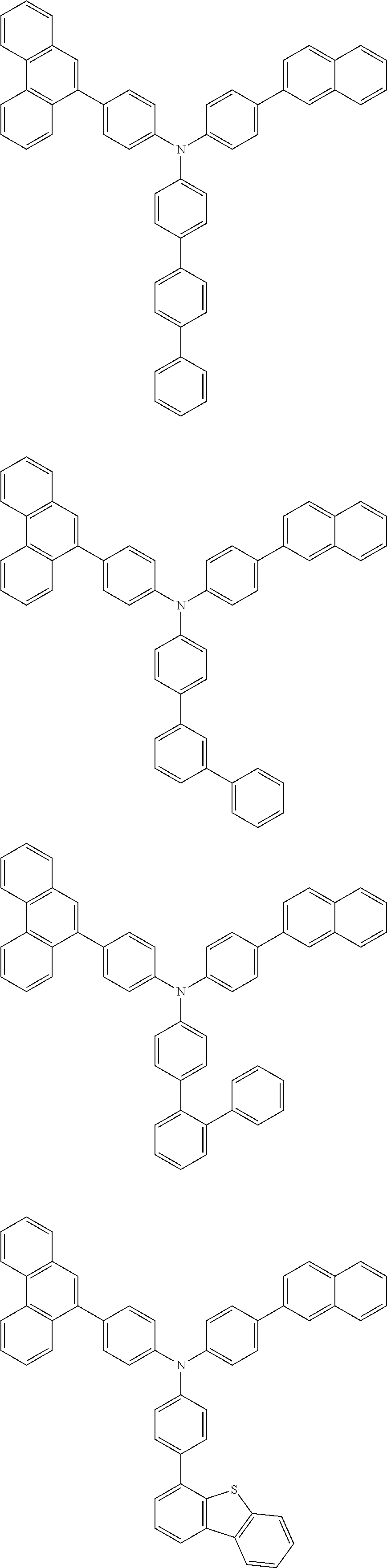 [SMILES: c1ccc(-c2ccc(-c3ccc(N(c4ccc(-c5ccc6ccccc6c5)cc4)c4ccc(-c5cc6ccccc6c6ccccc56)cc4)cc3)cc2)cc1 ; c1ccc(-c2cccc(-c3ccc(N(c4ccc(-c5ccc6ccccc6c5)cc4)c4ccc(-c5cc6ccccc6c6ccccc56)cc4)cc3)c2)cc1 ; c1ccc(-c2ccccc2-c2ccc(N(c3ccc(-c4ccc5ccccc5c4)cc3)c3ccc(-c4cc5ccccc5c5ccccc45)cc3)cc2)cc1 ; c1ccc2cc(-c3ccc(N(c4ccc(-c5cc6ccccc6c6ccccc56)cc4)c4ccc(-c5cccc6c5sc5ccccc56)cc4)cc3)ccc2c1]

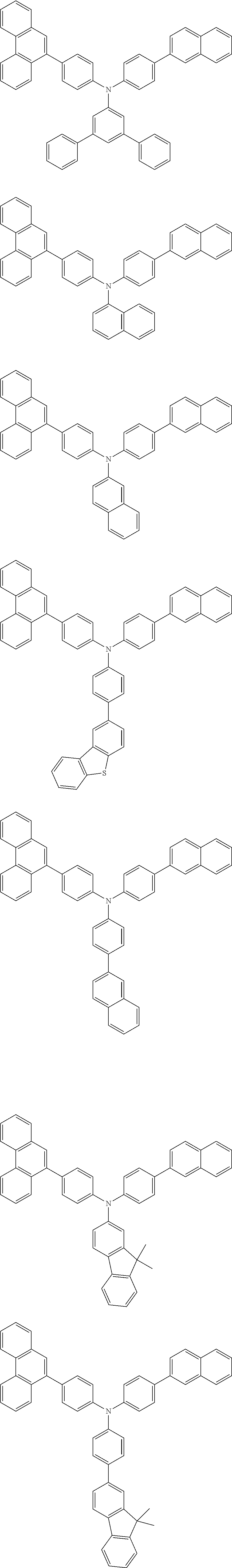

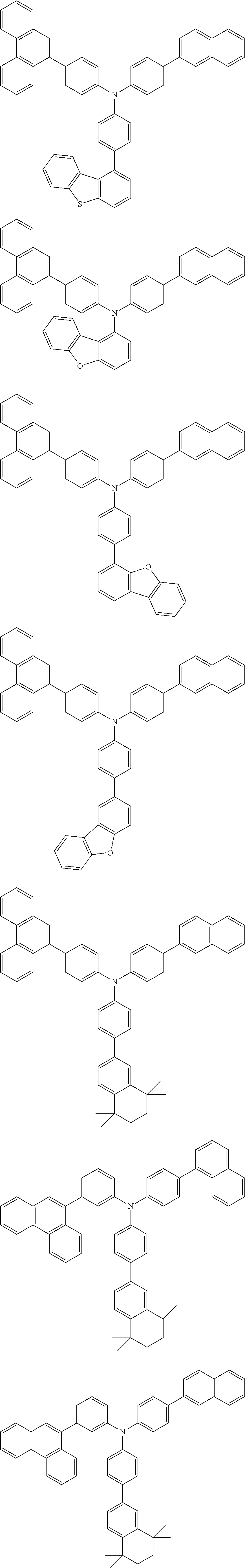

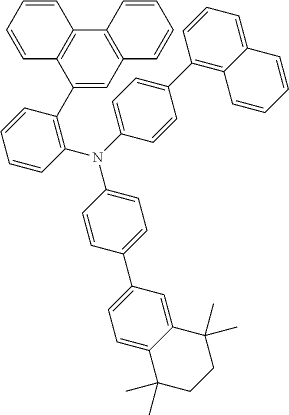 [SMILES: CC1(C)CCC(C)(C)c2cc(-c3ccc(N(c4ccc(-c5cccc6ccccc56)cc4)c4ccccc4-c4cc5ccccc5c5ccccc45)cc3)ccc21]

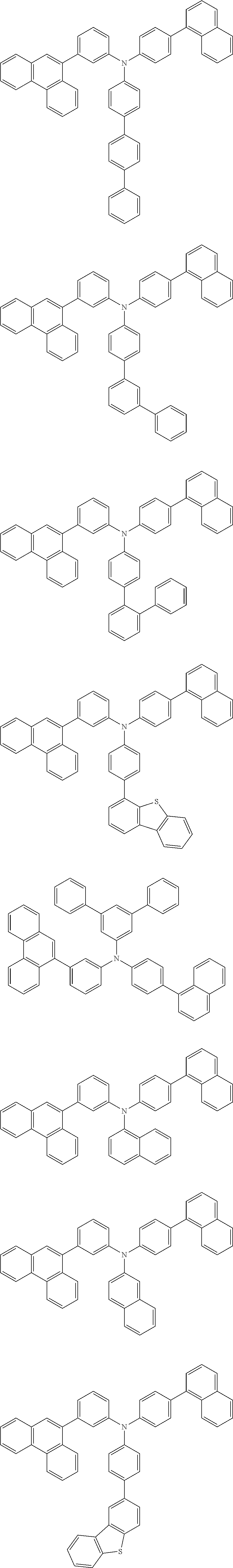

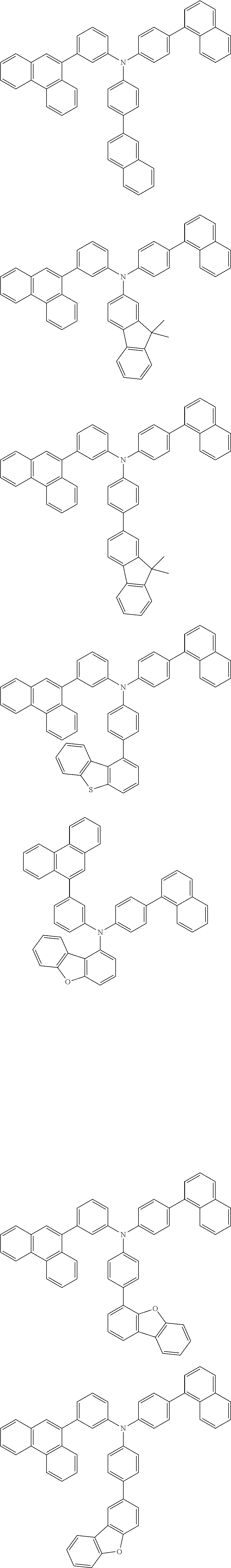

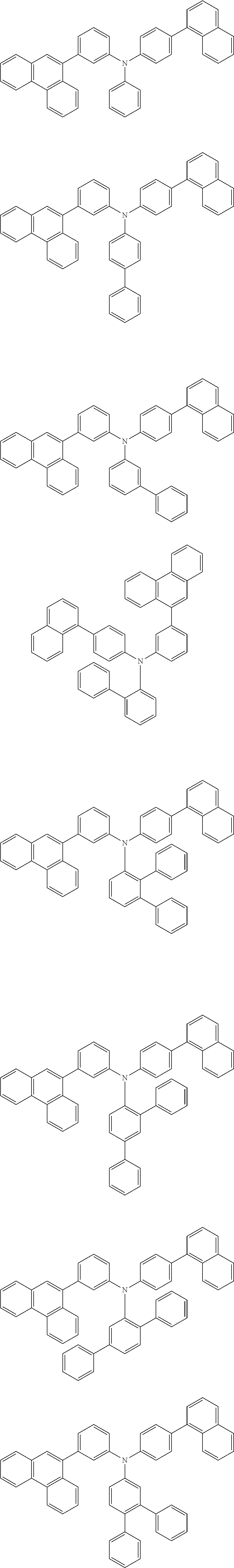

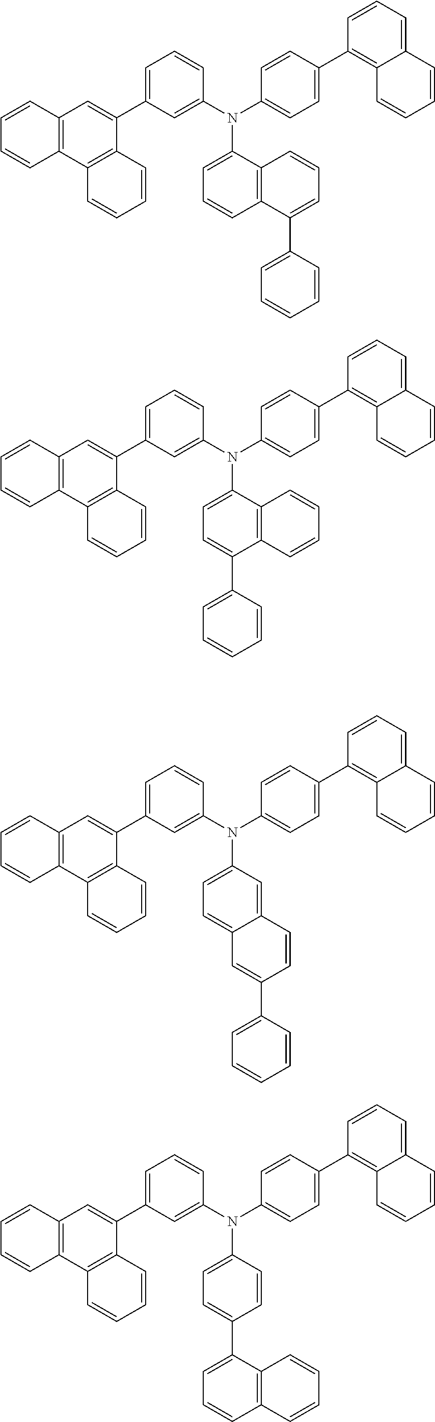 [SMILES: c1ccc(-c2cccc3c(N(c4ccc(-c5cccc6ccccc56)cc4)c4cccc(-c5cc6ccccc6c6ccccc56)c4)cccc23)cc1 ; c1ccc(-c2ccc(N(c3ccc(-c4cccc5ccccc45)cc3)c3cccc(-c4cc5ccccc5c5ccccc45)c3)c3ccccc23)cc1 ; c1ccc(-c2ccc3cc(N(c4ccc(-c5cccc6ccccc56)cc4)c4cccc(-c5cc6ccccc6c6ccccc56)c4)ccc3c2)cc1 ; c1cc(-c2cc3ccccc3c3ccccc23)cc(N(c2ccc(-c3cccc4ccccc34)cc2)c2ccc(-c3cccc4ccccc34)cc2)c1]

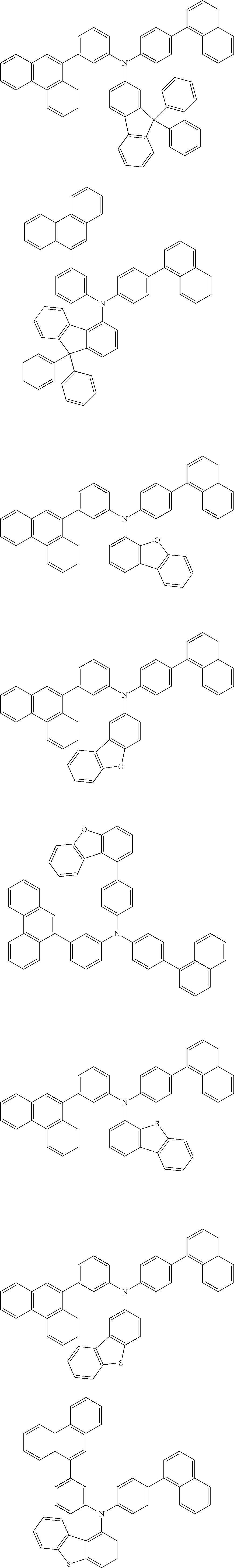

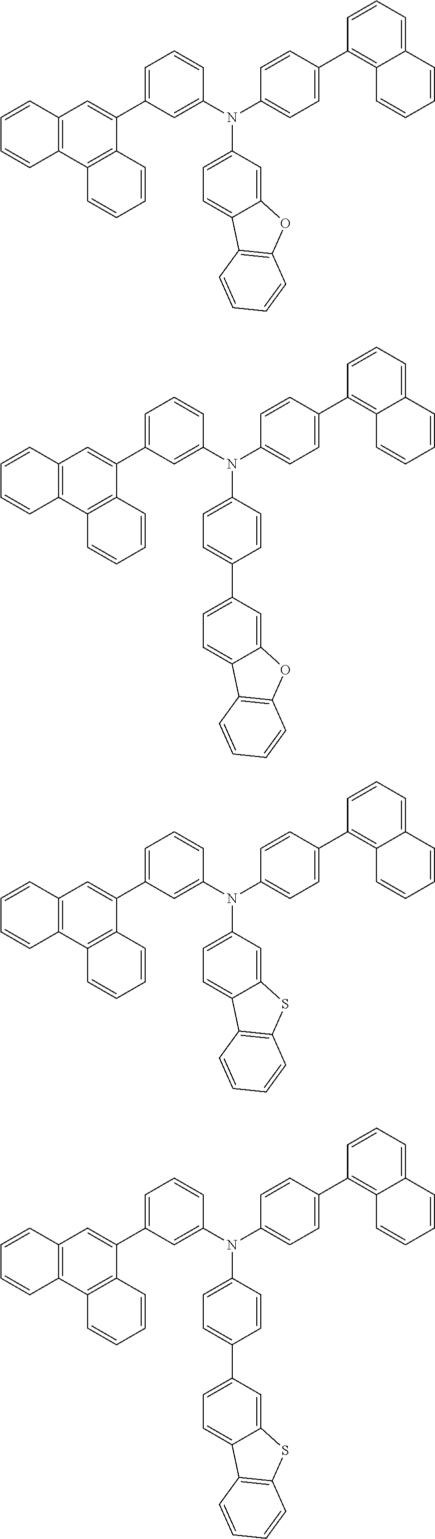 [SMILES: c1cc(-c2cc3ccccc3c3ccccc23)cc(N(c2ccc(-c3cccc4ccccc34)cc2)c2ccc3c(c2)oc2ccccc23)c1 ; c1cc(-c2cc3ccccc3c3ccccc23)cc(N(c2ccc(-c3ccc4c(c3)oc3ccccc34)cc2)c2ccc(-c3cccc4ccccc34)cc2)c1 ; c1cc(-c2cc3ccccc3c3ccccc23)cc(N(c2ccc(-c3cccc4ccccc34)cc2)c2ccc3c(c2)sc2ccccc23)c1 ; c1cc(-c2cc3ccccc3c3ccccc23)cc(N(c2ccc(-c3ccc4c(c3)sc3ccccc34)cc2)c2ccc(-c3cccc4ccccc34)cc2)c1]

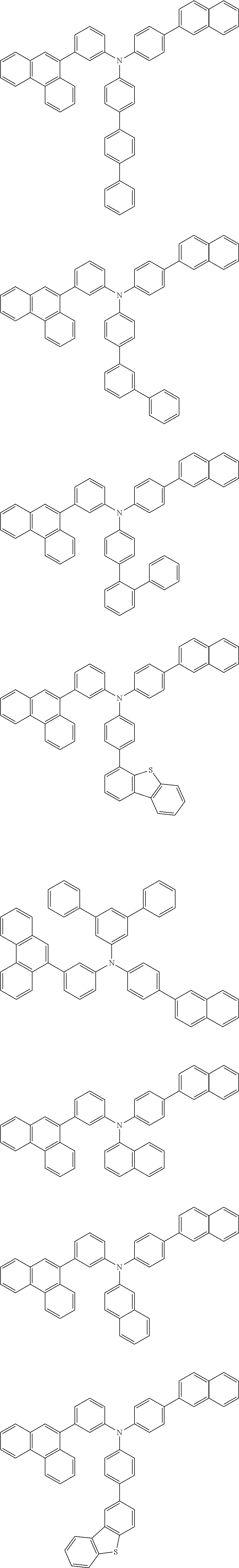

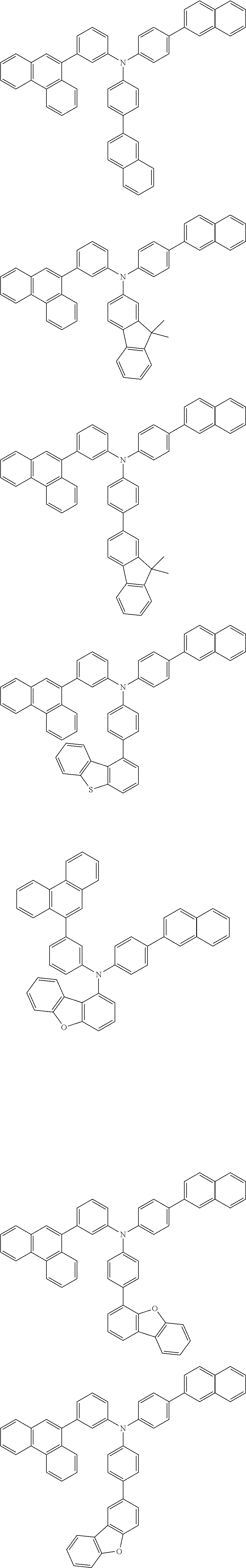

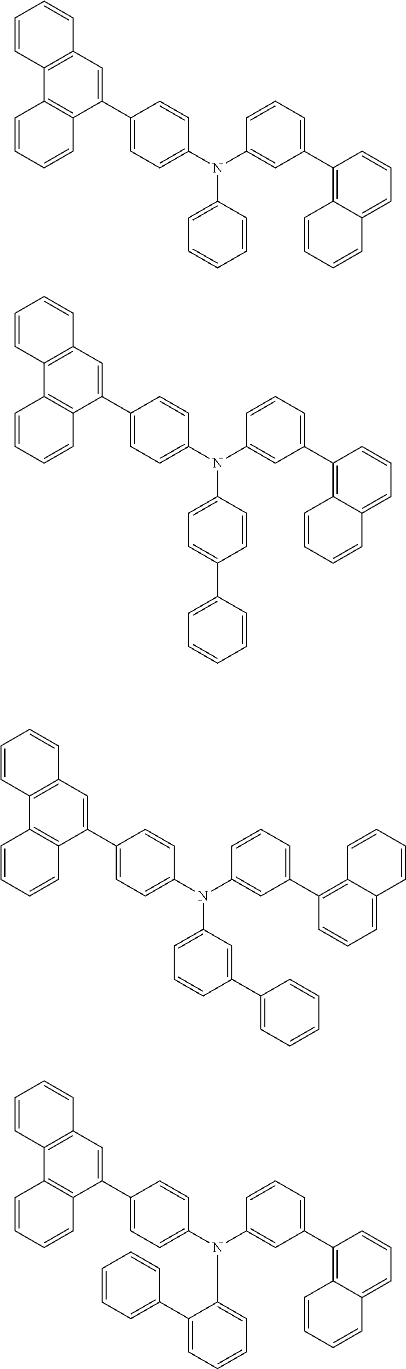 [SMILES: c1ccc(N(c2ccc(-c3cc4ccccc4c4ccccc34)cc2)c2cccc(-c3cccc4ccccc34)c2)cc1 ; c1ccc(-c2ccc(N(c3ccc(-c4cc5ccccc5c5ccccc45)cc3)c3cccc(-c4cccc5ccccc45)c3)cc2)cc1 ; c1ccc(-c2cccc(N(c3ccc(-c4cc5ccccc5c5ccccc45)cc3)c3cccc(-c4cccc5ccccc45)c3)c2)cc1 ; c1ccc(-c2ccccc2N(c2ccc(-c3cc4ccccc4c4ccccc34)cc2)c2cccc(-c3cccc4ccccc34)c2)cc1]

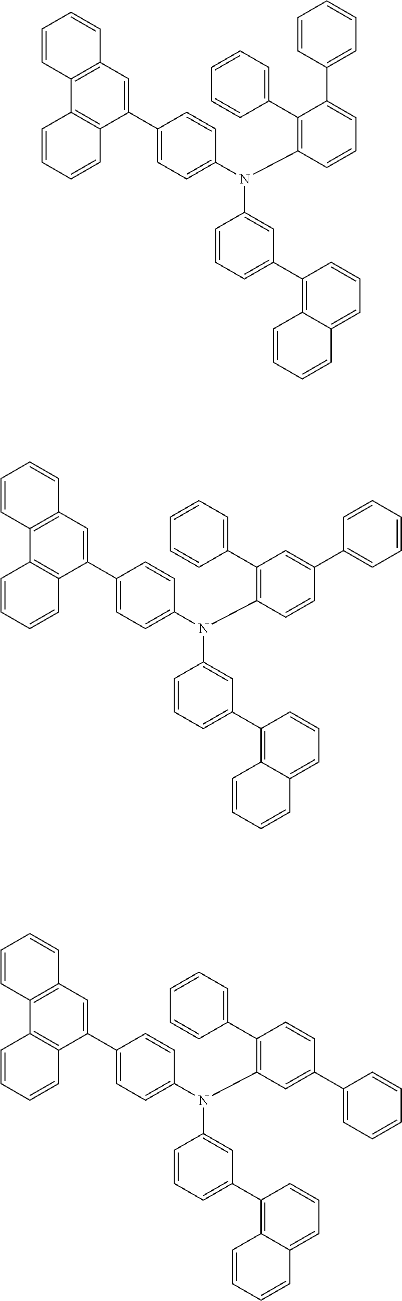 [SMILES: c1ccc(-c2cccc(N(c3ccc(-c4cc5ccccc5c5ccccc45)cc3)c3cccc(-c4cccc5ccccc45)c3)c2-c2ccccc2)cc1 ; c1ccc(-c2ccc(N(c3ccc(-c4cc5ccccc5c5ccccc45)cc3)c3cccc(-c4cccc5ccccc45)c3)c(-c3ccccc3)c2)cc1 ; c1ccc(-c2ccc(-c3ccccc3)c(N(c3ccc(-c4cc5ccccc5c5ccccc45)cc3)c3cccc(-c4cccc5ccccc45)c3)c2)cc1]

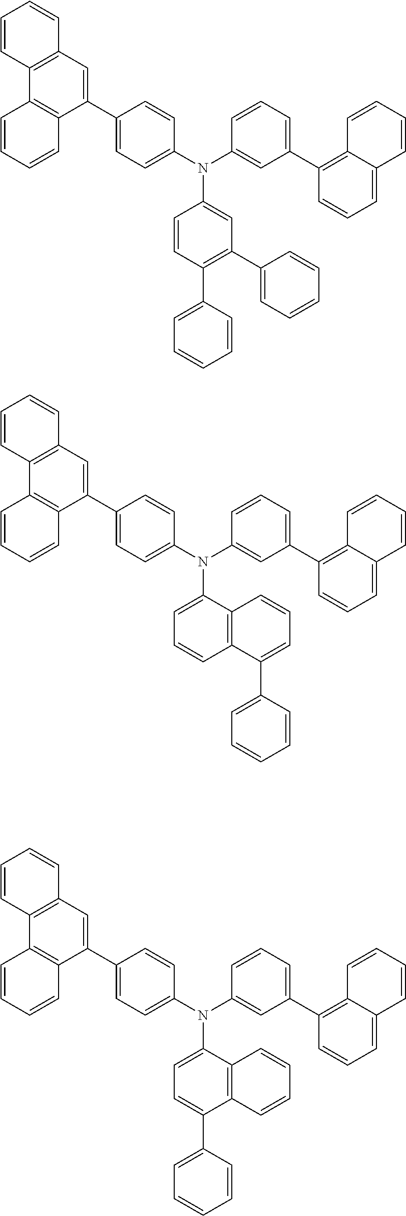 [SMILES: c1ccc(-c2ccc(N(c3ccc(-c4cc5ccccc5c5ccccc45)cc3)c3cccc(-c4cccc5ccccc45)c3)cc2-c2ccccc2)cc1 ; c1ccc(-c2cccc3c(N(c4ccc(-c5cc6ccccc6c6ccccc56)cc4)c4cccc(-c5cccc6ccccc56)c4)cccc23)cc1 ; c1ccc(-c2ccc(N(c3ccc(-c4cc5ccccc5c5ccccc45)cc3)c3cccc(-c4cccc5ccccc45)c3)c3ccccc23)cc1]

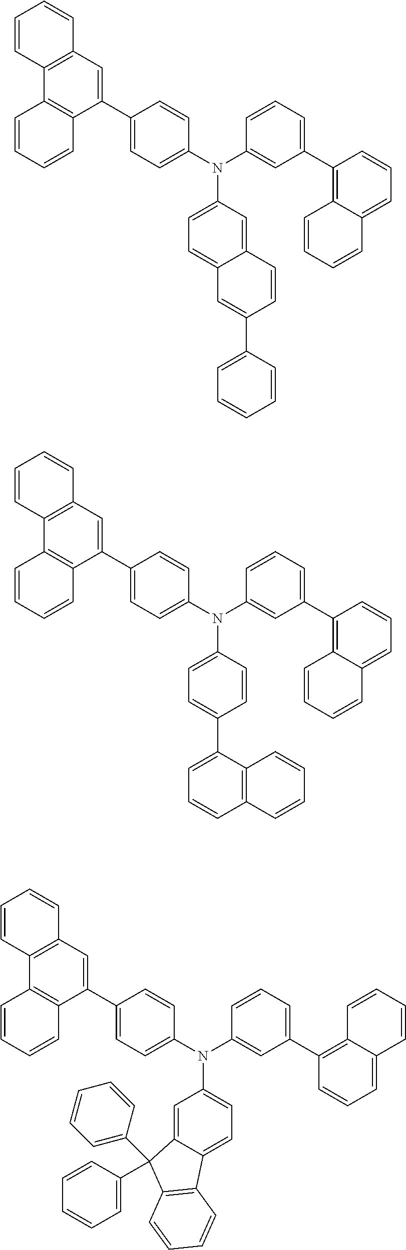 [SMILES: c1ccc(-c2ccc3cc(N(c4ccc(-c5cc6ccccc6c6ccccc56)cc4)c4cccc(-c5cccc6ccccc56)c4)ccc3c2)cc1 ; c1cc(-c2cccc3ccccc23)cc(N(c2ccc(-c3cccc4ccccc34)cc2)c2ccc(-c3cc4ccccc4c4ccccc34)cc2)c1 ; c1ccc(C2(c3ccccc3)c3ccccc3-c3ccc(N(c4ccc(-c5cc6ccccc6c6ccccc56)cc4)c4cccc(-c5cccc6ccccc56)c4)cc32)cc1]

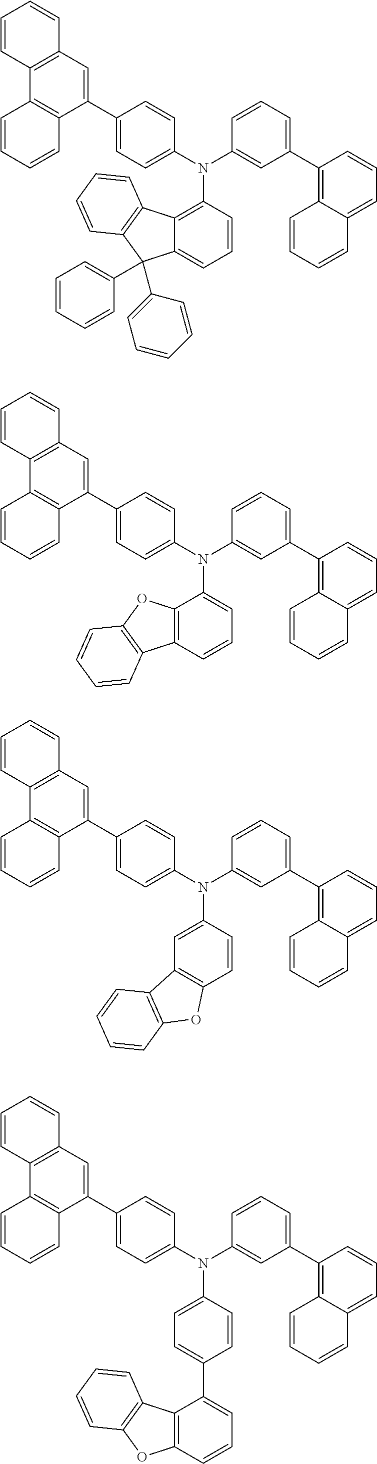 [SMILES: c1ccc(C2(c3ccccc3)c3ccccc3-c3c(N(c4ccc(-c5cc6ccccc6c6ccccc56)cc4)c4cccc(-c5cccc6ccccc56)c4)cccc32)cc1 ; c1cc(-c2cccc3ccccc23)cc(N(c2ccc(-c3cc4ccccc4c4ccccc34)cc2)c2cccc3c2oc2ccccc23)c1 ; c1cc(-c2cccc3ccccc23)cc(N(c2ccc(-c3cc4ccccc4c4ccccc34)cc2)c2ccc3oc4ccccc4c3c2)c1 ; c1cc(-c2cccc3ccccc23)cc(N(c2ccc(-c3cc4ccccc4c4ccccc34)cc2)c2ccc(-c3cccc4oc5ccccc5c34)cc2)c1]

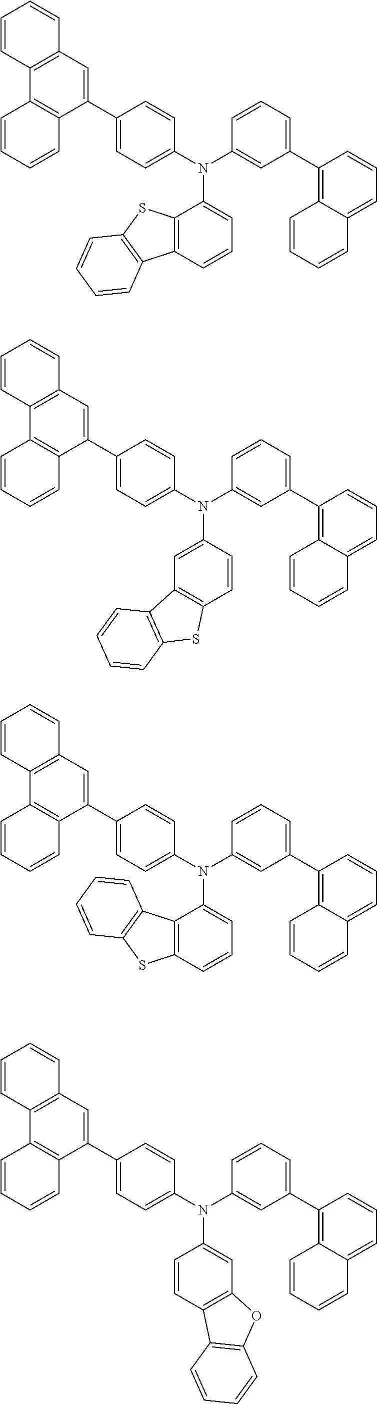 [SMILES: c1cc(-c2cccc3ccccc23)cc(N(c2ccc(-c3cc4ccccc4c4ccccc34)cc2)c2cccc3c2sc2ccccc23)c1 ; c1cc(-c2cccc3ccccc23)cc(N(c2ccc(-c3cc4ccccc4c4ccccc34)cc2)c2ccc3sc4ccccc4c3c2)c1 ; c1cc(-c2cccc3ccccc23)cc(N(c2ccc(-c3cc4ccccc4c4ccccc34)cc2)c2cccc3sc4ccccc4c23)c1 ; c1cc(-c2cccc3ccccc23)cc(N(c2ccc(-c3cc4ccccc4c4ccccc34)cc2)c2ccc3c(c2)oc2ccccc23)c1]

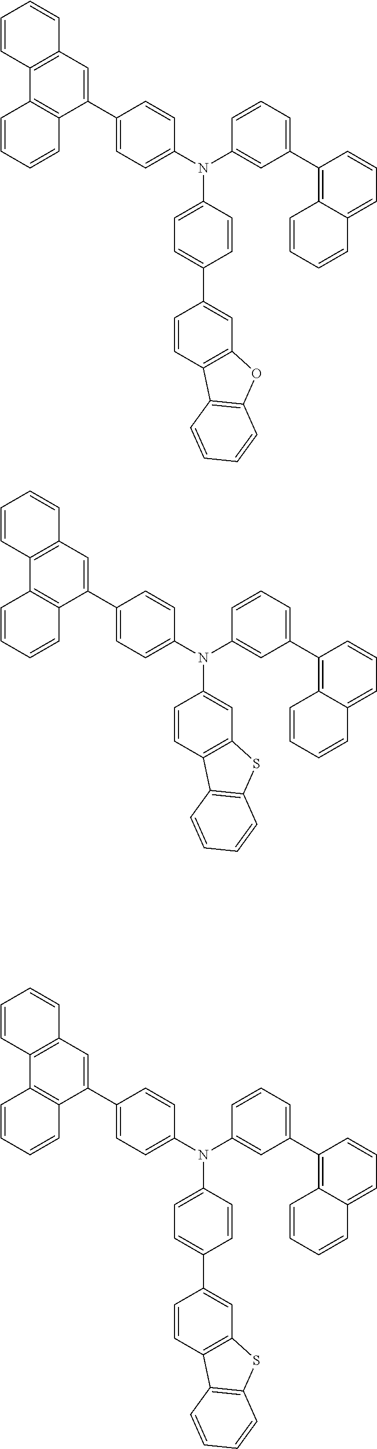 [SMILES: c1cc(-c2cccc3ccccc23)cc(N(c2ccc(-c3ccc4c(c3)oc3ccccc34)cc2)c2ccc(-c3cc4ccccc4c4ccccc34)cc2)c1 ; c1cc(-c2cccc3ccccc23)cc(N(c2ccc(-c3cc4ccccc4c4ccccc34)cc2)c2ccc3c(c2)sc2ccccc23)c1 ; c1cc(-c2cccc3ccccc23)cc(N(c2ccc(-c3ccc4c(c3)sc3ccccc34)cc2)c2ccc(-c3cc4ccccc4c4ccccc34)cc2)c1]

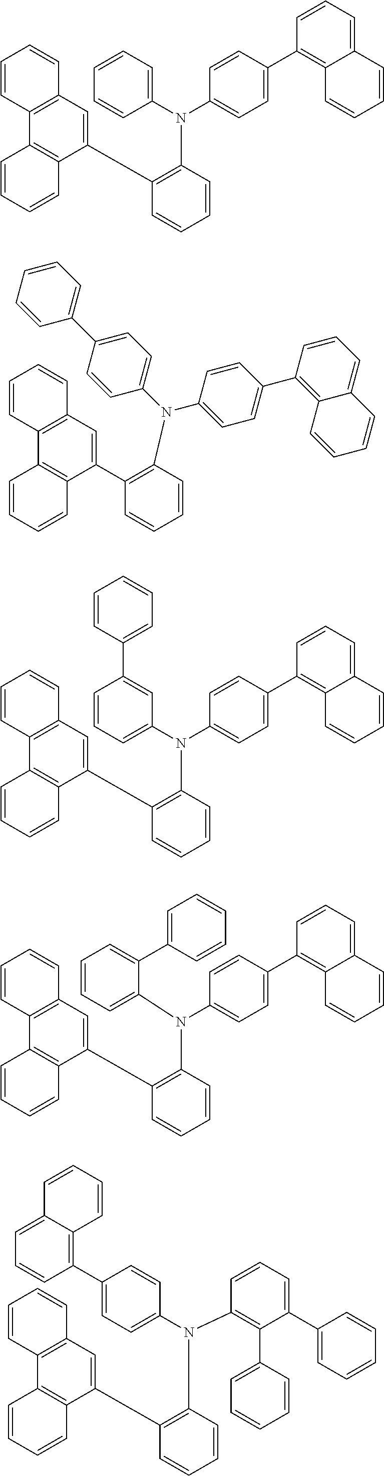

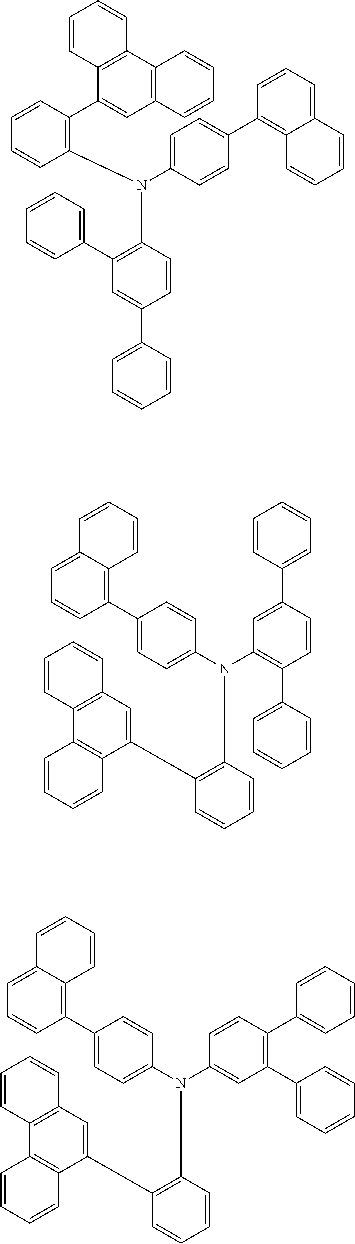 [SMILES: c1ccc(-c2ccc(N(c3ccc(-c4cccc5ccccc45)cc3)c3ccccc3-c3cc4ccccc4c4ccccc34)c(-c3ccccc3)c2)cc1 ; c1ccc(-c2ccc(-c3ccccc3)c(N(c3ccc(-c4cccc5ccccc45)cc3)c3ccccc3-c3cc4ccccc4c4ccccc34)c2)cc1 ; c1ccc(-c2ccc(N(c3ccc(-c4cccc5ccccc45)cc3)c3ccccc3-c3cc4ccccc4c4ccccc34)cc2-c2ccccc2)cc1]

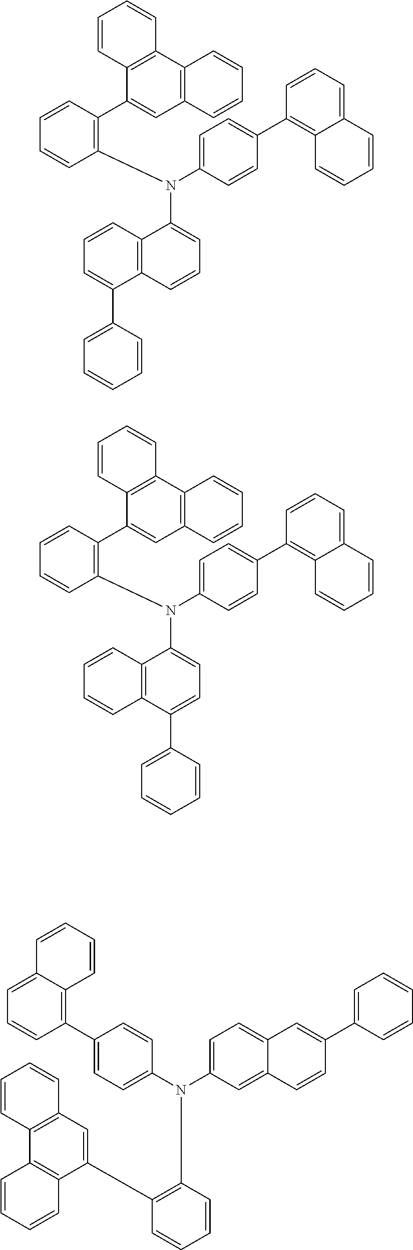

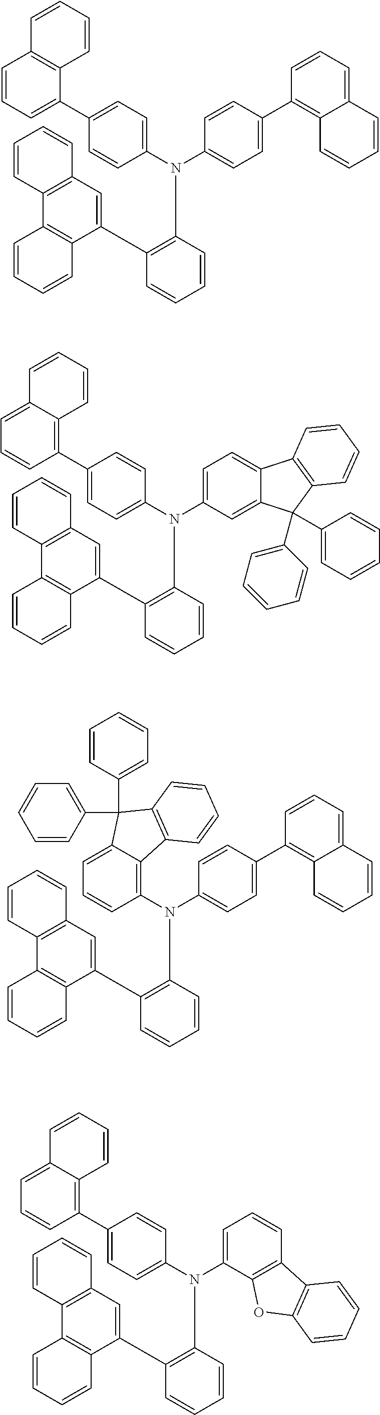 [SMILES: c1ccc(N(c2ccc(-c3cccc4ccccc34)cc2)c2ccc(-c3cccc4ccccc34)cc2)c(-c2cc3ccccc3c3ccccc23)c1 ; c1ccc(C2(c3ccccc3)c3ccccc3-c3ccc(N(c4ccc(-c5cccc6ccccc56)cc4)c4ccccc4-c4cc5ccccc5c5ccccc45)cc32)cc1 ; c1ccc(C2(c3ccccc3)c3ccccc3-c3c(N(c4ccc(-c5cccc6ccccc56)cc4)c4ccccc4-c4cc5ccccc5c5ccccc45)cccc32)cc1 ; c1ccc(N(c2ccc(-c3cccc4ccccc34)cc2)c2cccc3c2oc2ccccc23)c(-c2cc3ccccc3c3ccccc23)c1]

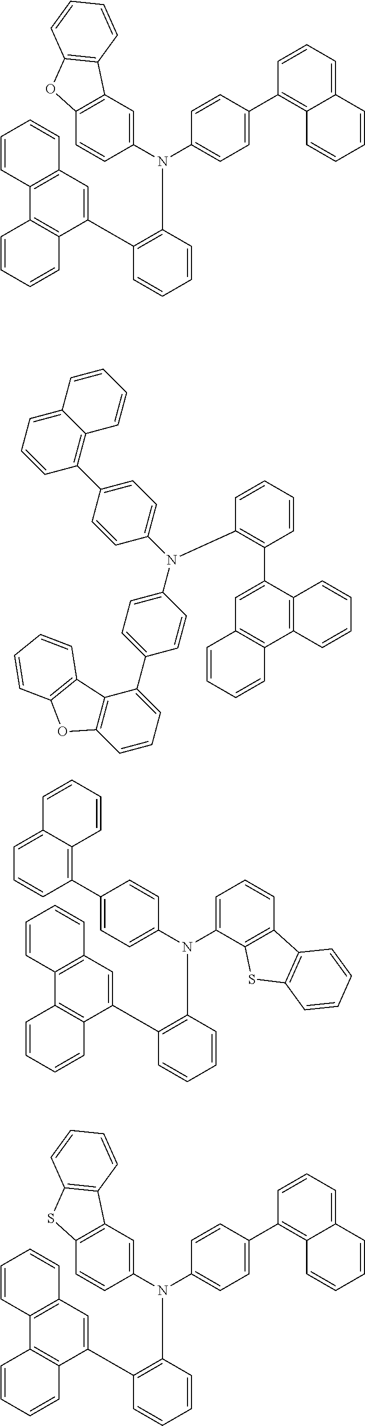 [SMILES: c1ccc(N(c2ccc(-c3cccc4ccccc34)cc2)c2ccc3oc4ccccc4c3c2)c(-c2cc3ccccc3c3ccccc23)c1 ; c1ccc(N(c2ccc(-c3cccc4ccccc34)cc2)c2ccc(-c3cccc4oc5ccccc5c34)cc2)c(-c2cc3ccccc3c3ccccc23)c1 ; c1ccc(N(c2ccc(-c3cccc4ccccc34)cc2)c2cccc3c2sc2ccccc23)c(-c2cc3ccccc3c3ccccc23)c1 ; c1ccc(N(c2ccc(-c3cccc4ccccc34)cc2)c2ccc3sc4ccccc4c3c2)c(-c2cc3ccccc3c3ccccc23)c1]

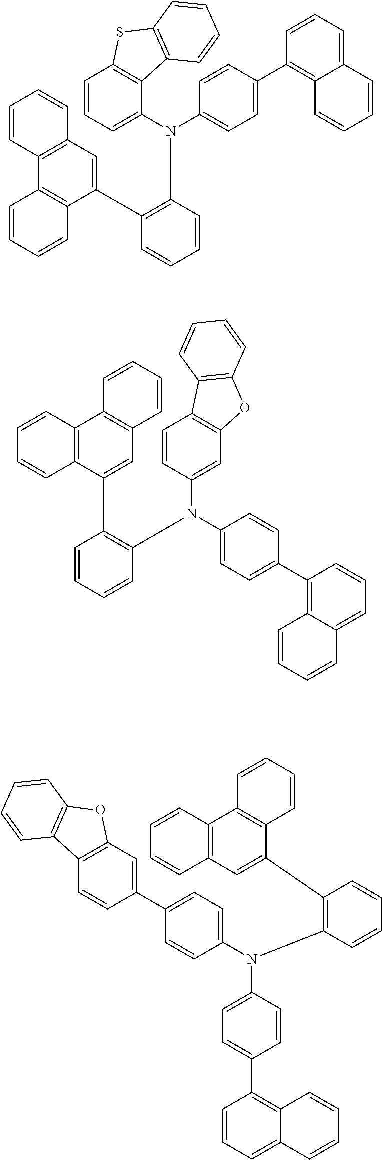 [SMILES: c1ccc(N(c2ccc(-c3cccc4ccccc34)cc2)c2cccc3sc4ccccc4c23)c(-c2cc3ccccc3c3ccccc23)c1 ; c1ccc(N(c2ccc(-c3cccc4ccccc34)cc2)c2ccc3c(c2)oc2ccccc23)c(-c2cc3ccccc3c3ccccc23)c1 ; c1ccc(N(c2ccc(-c3ccc4c(c3)oc3ccccc34)cc2)c2ccc(-c3cccc4ccccc34)cc2)c(-c2cc3ccccc3c3ccccc23)c1]

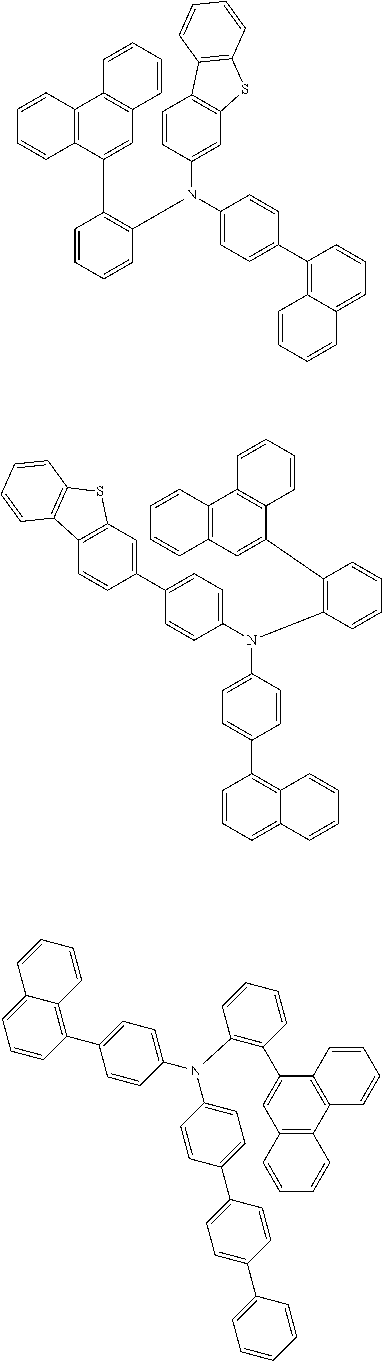 [SMILES: c1ccc(N(c2ccc(-c3cccc4ccccc34)cc2)c2ccc3c(c2)sc2ccccc23)c(-c2cc3ccccc3c3ccccc23)c1 ; c1ccc(N(c2ccc(-c3ccc4c(c3)sc3ccccc34)cc2)c2ccc(-c3cccc4ccccc34)cc2)c(-c2cc3ccccc3c3ccccc23)c1 ; c1ccc(-c2ccc(-c3ccc(N(c4ccc(-c5cccc6ccccc56)cc4)c4ccccc4-c4cc5ccccc5c5ccccc45)cc3)cc2)cc1]

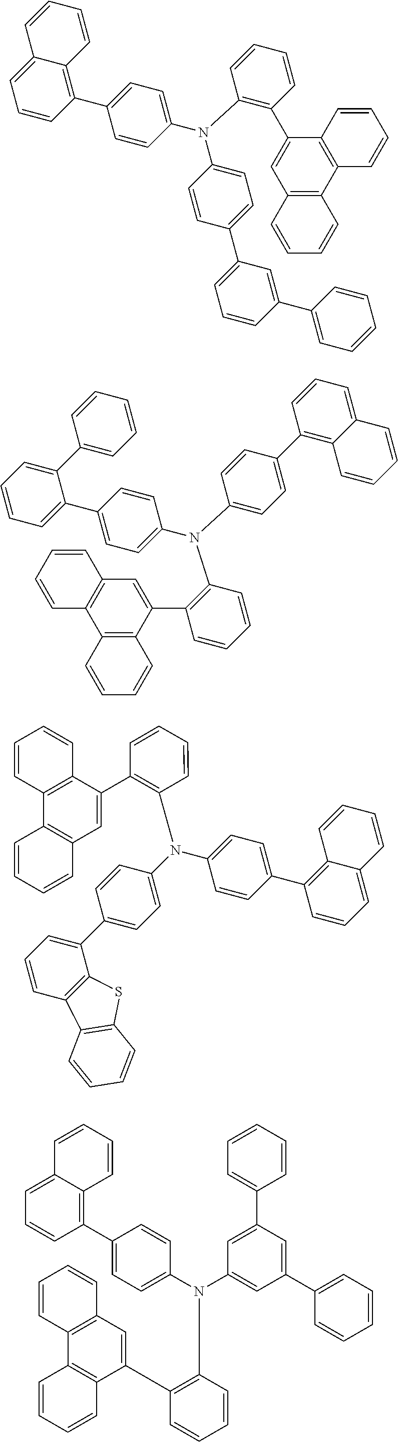

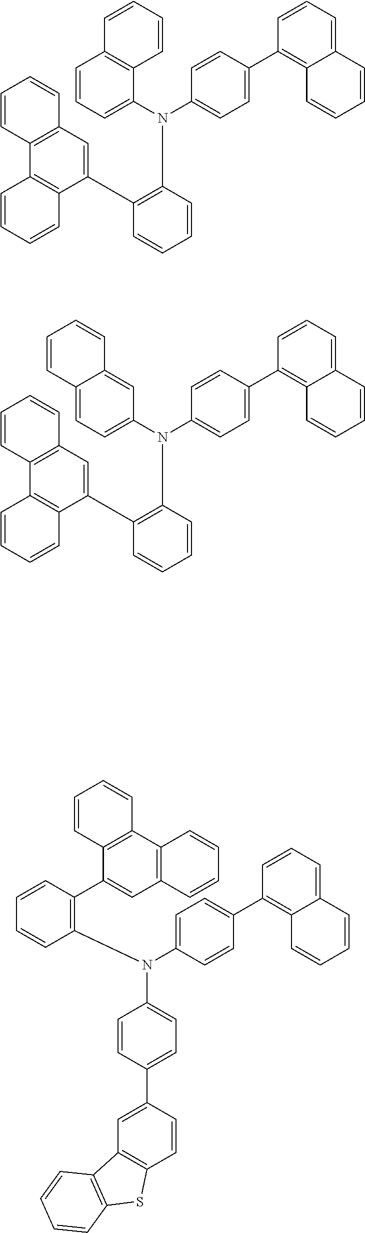 [SMILES: c1ccc(N(c2ccc(-c3cccc4ccccc34)cc2)c2cccc3ccccc23)c(-c2cc3ccccc3c3ccccc23)c1 ; c1ccc(N(c2ccc(-c3cccc4ccccc34)cc2)c2ccc3ccccc3c2)c(-c2cc3ccccc3c3ccccc23)c1 ; c1ccc(N(c2ccc(-c3ccc4sc5ccccc5c4c3)cc2)c2ccc(-c3cccc4ccccc34)cc2)c(-c2cc3ccccc3c3ccccc23)c1]

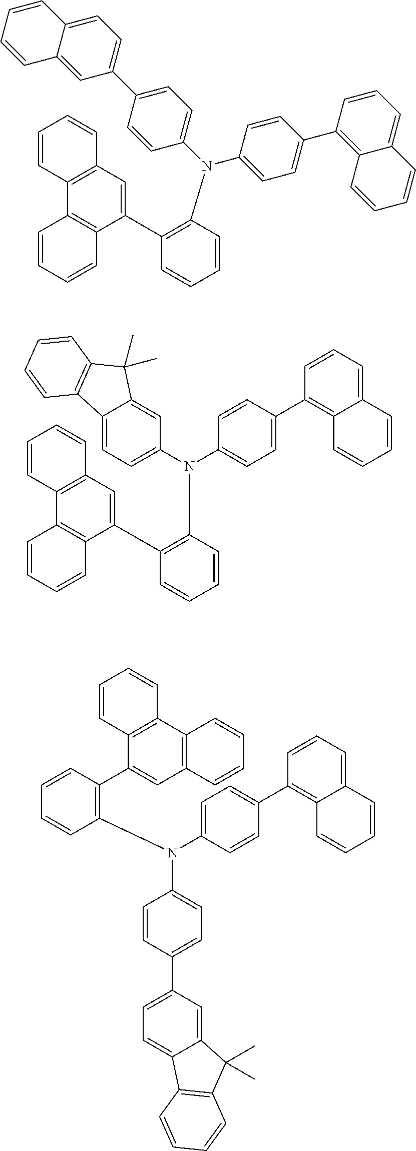 [SMILES: c1ccc(N(c2ccc(-c3ccc4ccccc4c3)cc2)c2ccc(-c3cccc4ccccc34)cc2)c(-c2cc3ccccc3c3ccccc23)c1 ; CC1(C)c2ccccc2-c2ccc(N(c3ccc(-c4cccc5ccccc45)cc3)c3ccccc3-c3cc4ccccc4c4ccccc34)cc21 ; CC1(C)c2ccccc2-c2ccc(-c3ccc(N(c4ccc(-c5cccc6ccccc56)cc4)c4ccccc4-c4cc5ccccc5c5ccccc45)cc3)cc21]

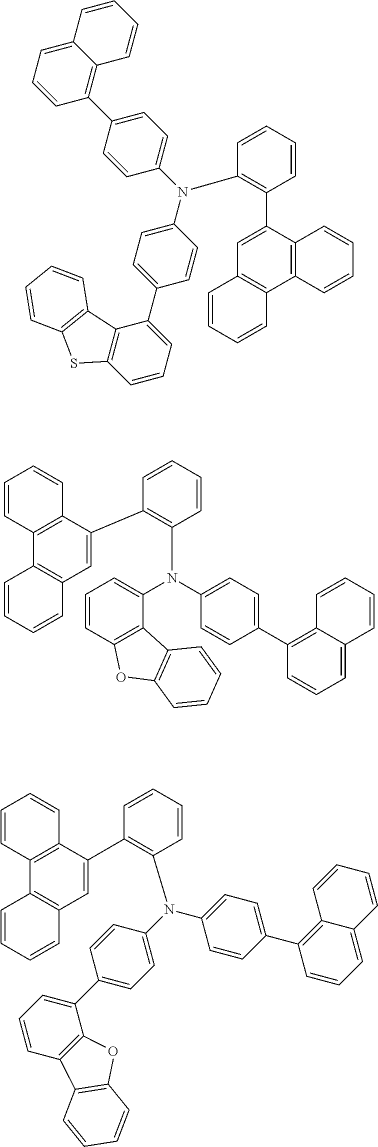 [SMILES: c1ccc(N(c2ccc(-c3cccc4ccccc34)cc2)c2ccc(-c3cccc4sc5ccccc5c34)cc2)c(-c2cc3ccccc3c3ccccc23)c1 ; c1ccc(N(c2ccc(-c3cccc4ccccc34)cc2)c2cccc3oc4ccccc4c23)c(-c2cc3ccccc3c3ccccc23)c1 ; c1ccc(N(c2ccc(-c3cccc4ccccc34)cc2)c2ccc(-c3cccc4c3oc3ccccc34)cc2)c(-c2cc3ccccc3c3ccccc23)c1]

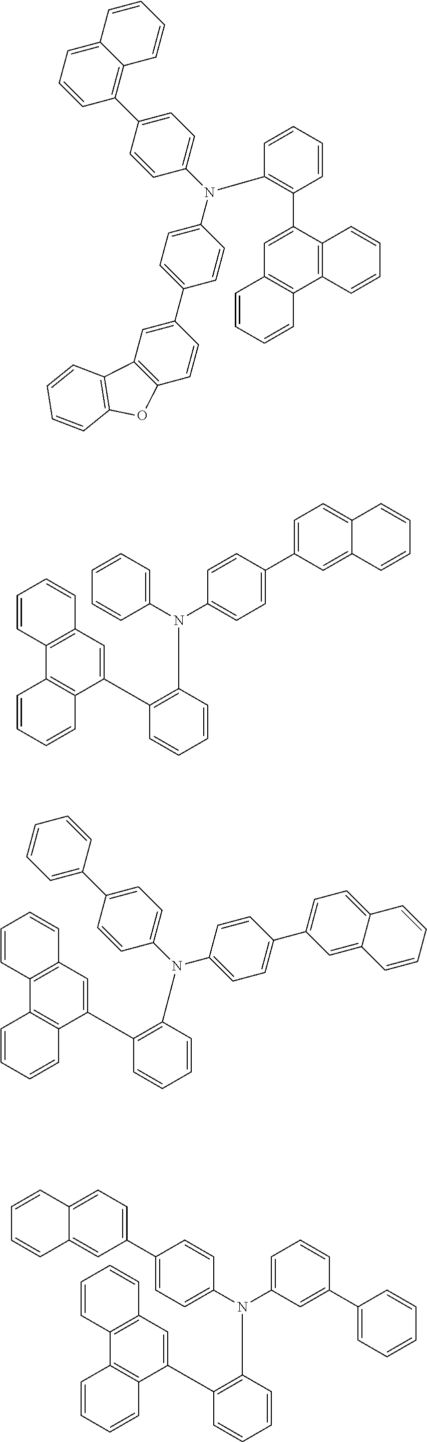 [SMILES: c1ccc(N(c2ccc(-c3ccc4oc5ccccc5c4c3)cc2)c2ccc(-c3cccc4ccccc34)cc2)c(-c2cc3ccccc3c3ccccc23)c1 ; c1ccc(N(c2ccc(-c3ccc4ccccc4c3)cc2)c2ccccc2-c2cc3ccccc3c3ccccc23)cc1 ; c1ccc(-c2ccc(N(c3ccc(-c4ccc5ccccc5c4)cc3)c3ccccc3-c3cc4ccccc4c4ccccc34)cc2)cc1 ; c1ccc(-c2cccc(N(c3ccc(-c4ccc5ccccc5c4)cc3)c3ccccc3-c3cc4ccccc4c4ccccc34)c2)cc1]

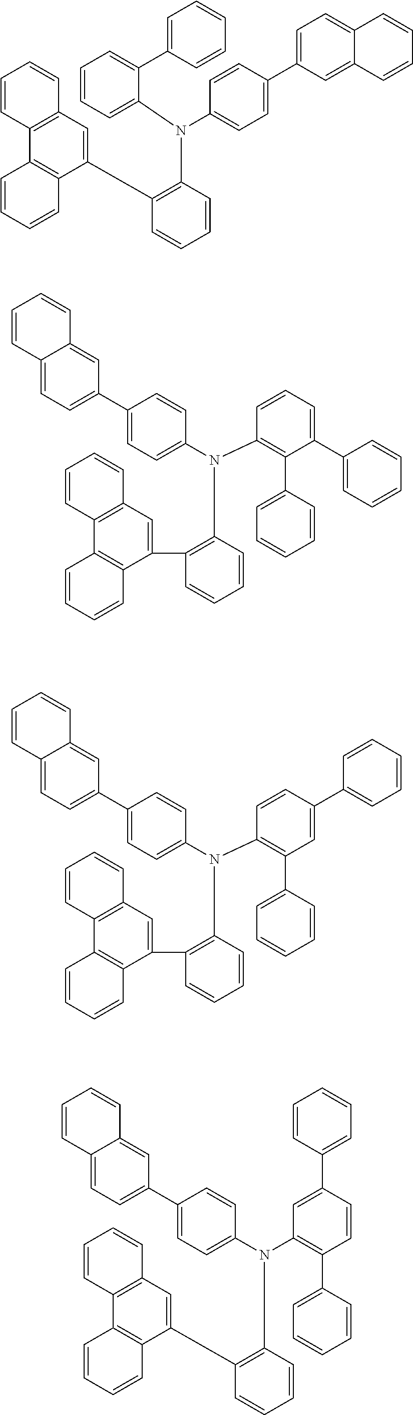 [SMILES: c1ccc(-c2ccccc2N(c2ccc(-c3ccc4ccccc4c3)cc2)c2ccccc2-c2cc3ccccc3c3ccccc23)cc1 ; c1ccc(-c2cccc(N(c3ccc(-c4ccc5ccccc5c4)cc3)c3ccccc3-c3cc4ccccc4c4ccccc34)c2-c2ccccc2)cc1 ; c1ccc(-c2ccc(N(c3ccc(-c4ccc5ccccc5c4)cc3)c3ccccc3-c3cc4ccccc4c4ccccc34)c(-c3ccccc3)c2)cc1 ; c1ccc(-c2ccc(-c3ccccc3)c(N(c3ccc(-c4ccc5ccccc5c4)cc3)c3ccccc3-c3cc4ccccc4c4ccccc34)c2)cc1]

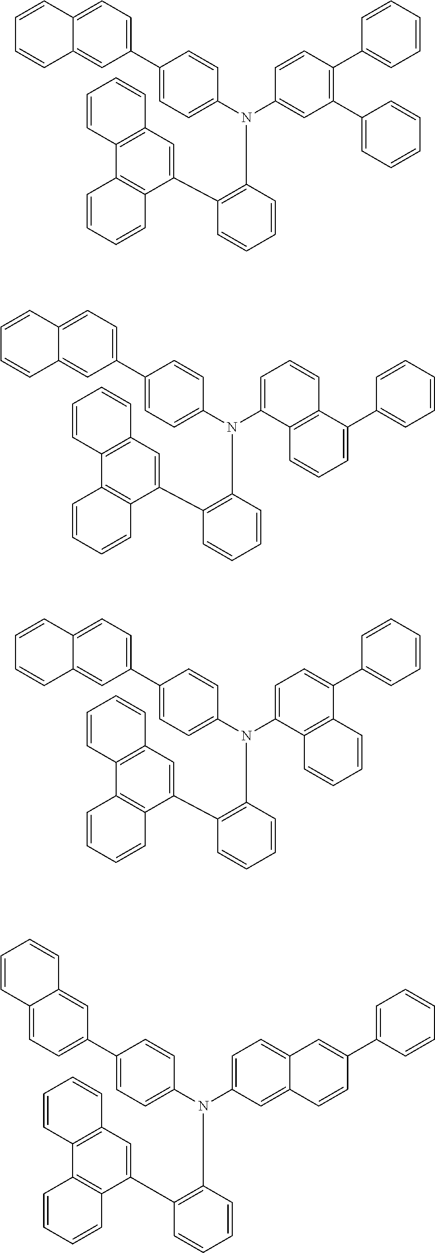 [SMILES: c1ccc(-c2ccc(N(c3ccc(-c4ccc5ccccc5c4)cc3)c3ccccc3-c3cc4ccccc4c4ccccc34)cc2-c2ccccc2)cc1 ; c1ccc(-c2cccc3c(N(c4ccc(-c5ccc6ccccc6c5)cc4)c4ccccc4-c4cc5ccccc5c5ccccc45)cccc23)cc1 ; c1ccc(-c2ccc(N(c3ccc(-c4ccc5ccccc5c4)cc3)c3ccccc3-c3cc4ccccc4c4ccccc34)c3ccccc23)cc1 ; c1ccc(-c2ccc3cc(N(c4ccc(-c5ccc6ccccc6c5)cc4)c4ccccc4-c4cc5ccccc5c5ccccc45)ccc3c2)cc1]

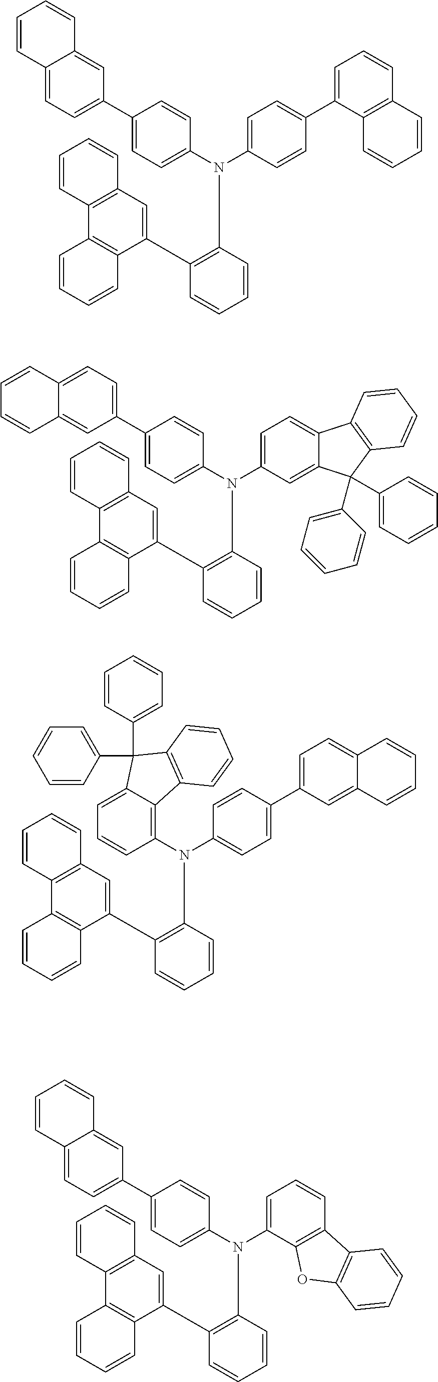 [SMILES: c1ccc(N(c2ccc(-c3ccc4ccccc4c3)cc2)c2ccc(-c3cccc4ccccc34)cc2)c(-c2cc3ccccc3c3ccccc23)c1 ; c1ccc(C2(c3ccccc3)c3ccccc3-c3ccc(N(c4ccc(-c5ccc6ccccc6c5)cc4)c4ccccc4-c4cc5ccccc5c5ccccc45)cc32)cc1 ; c1ccc(C2(c3ccccc3)c3ccccc3-c3c(N(c4ccc(-c5ccc6ccccc6c5)cc4)c4ccccc4-c4cc5ccccc5c5ccccc45)cccc32)cc1 ; c1ccc(N(c2ccc(-c3ccc4ccccc4c3)cc2)c2cccc3c2oc2ccccc23)c(-c2cc3ccccc3c3ccccc23)c1]

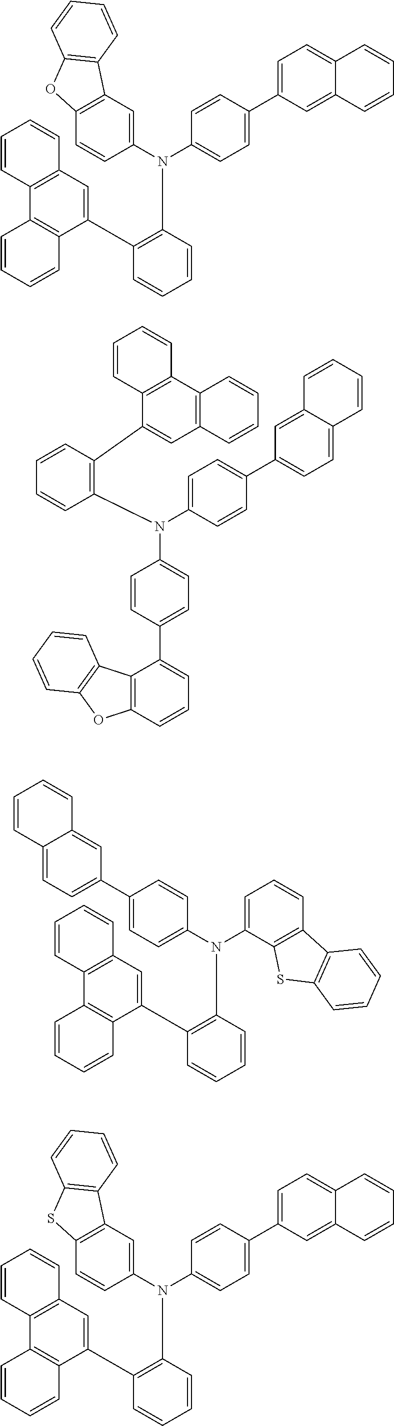

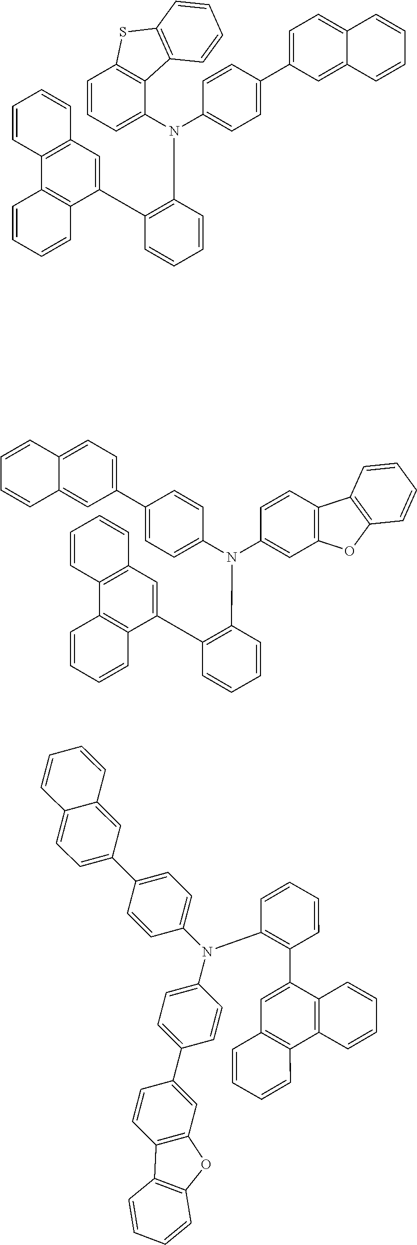 [SMILES: c1ccc(N(c2ccc(-c3ccc4ccccc4c3)cc2)c2cccc3sc4ccccc4c23)c(-c2cc3ccccc3c3ccccc23)c1 ; c1ccc(N(c2ccc(-c3ccc4ccccc4c3)cc2)c2ccc3c(c2)oc2ccccc23)c(-c2cc3ccccc3c3ccccc23)c1 ; c1ccc(N(c2ccc(-c3ccc4ccccc4c3)cc2)c2ccc(-c3ccc4c(c3)oc3ccccc34)cc2)c(-c2cc3ccccc3c3ccccc23)c1]

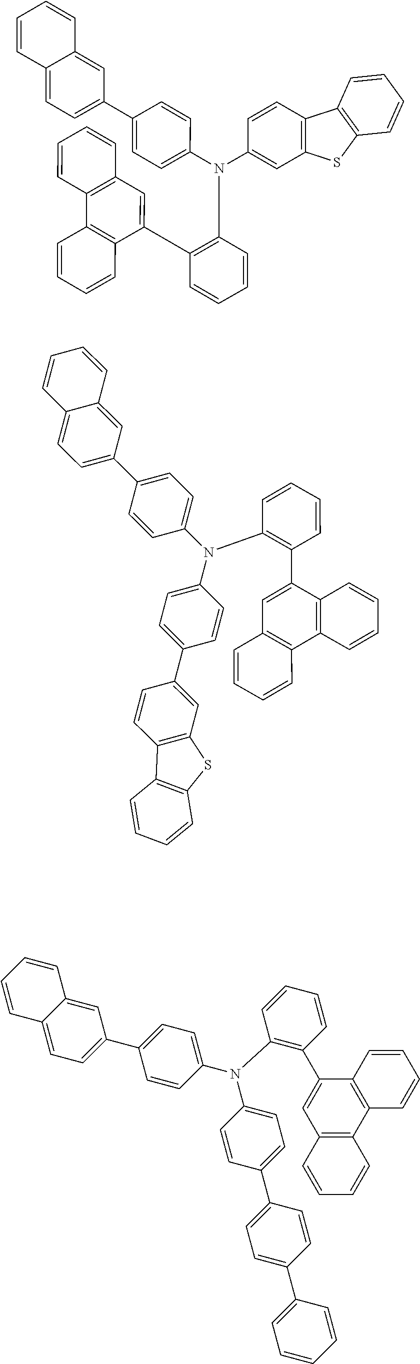 [SMILES: c1ccc(N(c2ccc(-c3ccc4ccccc4c3)cc2)c2ccc3c(c2)sc2ccccc23)c(-c2cc3ccccc3c3ccccc23)c1 ; c1ccc(N(c2ccc(-c3ccc4ccccc4c3)cc2)c2ccc(-c3ccc4c(c3)sc3ccccc34)cc2)c(-c2cc3ccccc3c3ccccc23)c1 ; c1ccc(-c2ccc(-c3ccc(N(c4ccc(-c5ccc6ccccc6c5)cc4)c4ccccc4-c4cc5ccccc5c5ccccc45)cc3)cc2)cc1]

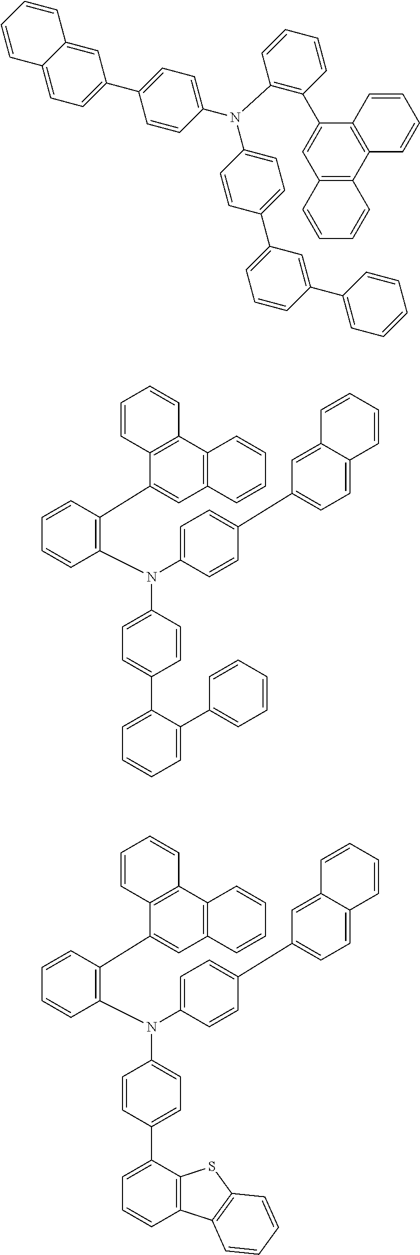 [SMILES: c1ccc(-c2cccc(-c3ccc(N(c4ccc(-c5ccc6ccccc6c5)cc4)c4ccccc4-c4cc5ccccc5c5ccccc45)cc3)c2)cc1 ; c1ccc(-c2ccccc2-c2ccc(N(c3ccc(-c4ccc5ccccc5c4)cc3)c3ccccc3-c3cc4ccccc4c4ccccc34)cc2)cc1 ; c1ccc(N(c2ccc(-c3ccc4ccccc4c3)cc2)c2ccc(-c3cccc4c3sc3ccccc34)cc2)c(-c2cc3ccccc3c3ccccc23)c1]

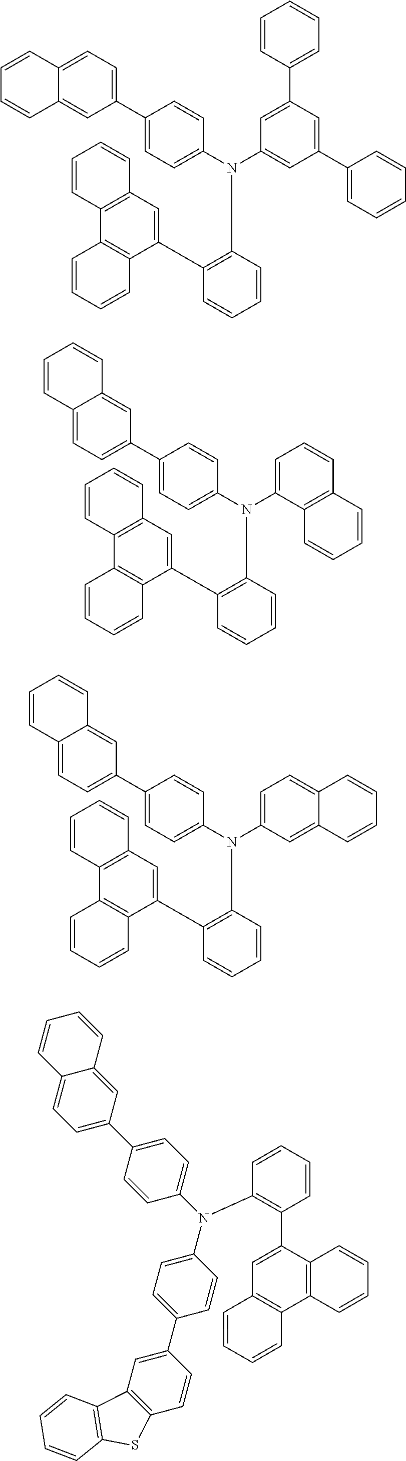

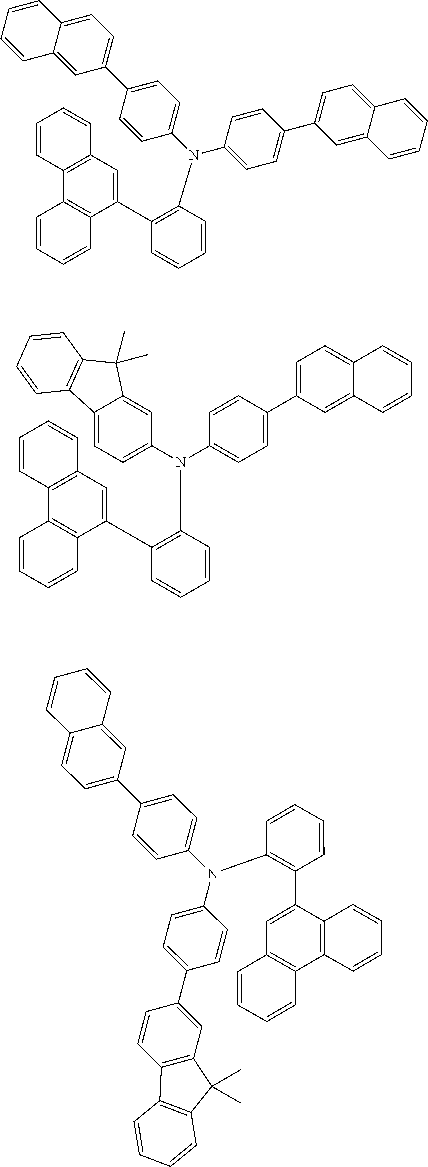 [SMILES: c1ccc(N(c2ccc(-c3ccc4ccccc4c3)cc2)c2ccc(-c3ccc4ccccc4c3)cc2)c(-c2cc3ccccc3c3ccccc23)c1 ; CC1(C)c2ccccc2-c2ccc(N(c3ccc(-c4ccc5ccccc5c4)cc3)c3ccccc3-c3cc4ccccc4c4ccccc34)cc21 ; CC1(C)c2ccccc2-c2ccc(-c3ccc(N(c4ccc(-c5ccc6ccccc6c5)cc4)c4ccccc4-c4cc5ccccc5c5ccccc45)cc3)cc21]

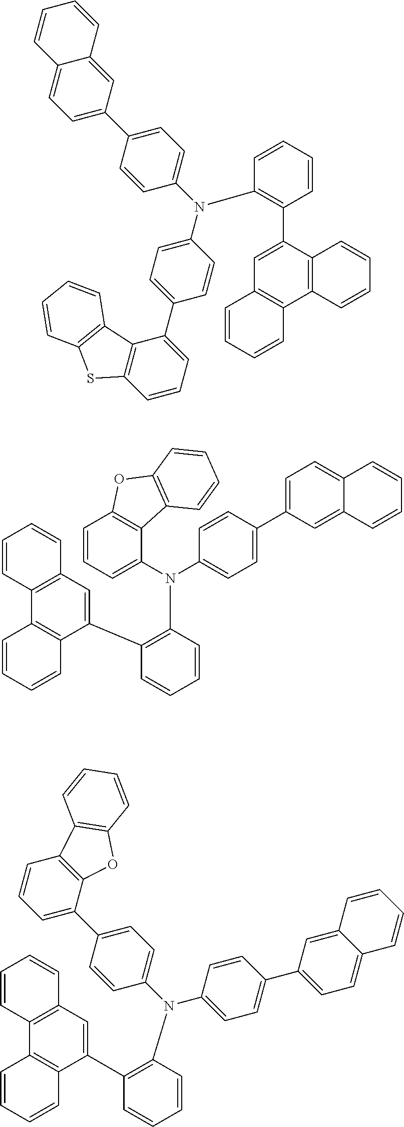 [SMILES: c1ccc(N(c2ccc(-c3ccc4ccccc4c3)cc2)c2ccc(-c3cccc4sc5ccccc5c34)cc2)c(-c2cc3ccccc3c3ccccc23)c1 ; c1ccc(N(c2ccc(-c3ccc4ccccc4c3)cc2)c2cccc3oc4ccccc4c23)c(-c2cc3ccccc3c3ccccc23)c1 ; c1ccc(N(c2ccc(-c3ccc4ccccc4c3)cc2)c2ccc(-c3cccc4c3oc3ccccc34)cc2)c(-c2cc3ccccc3c3ccccc23)c1]

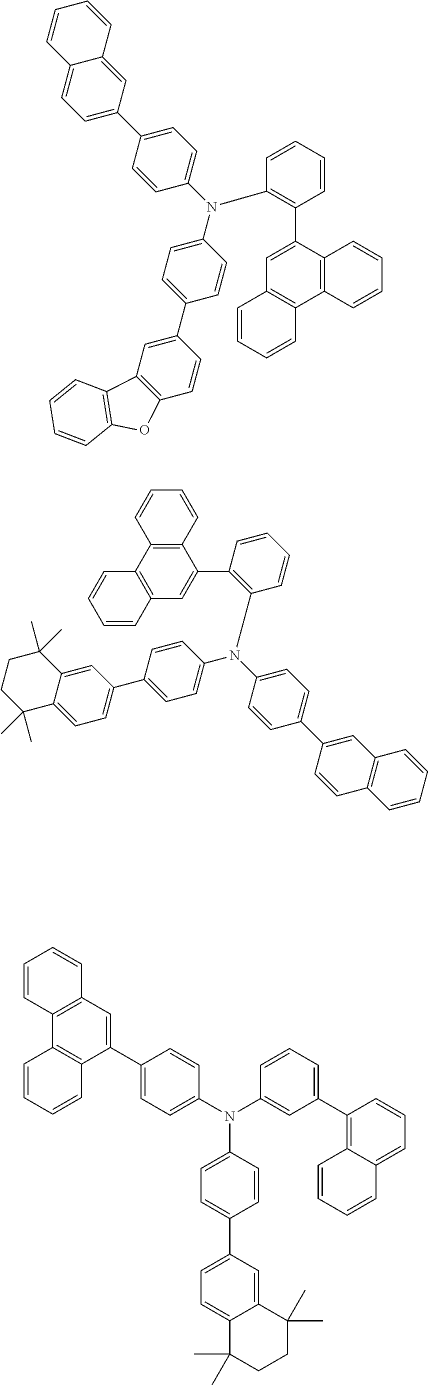 [SMILES: c1ccc(N(c2ccc(-c3ccc4ccccc4c3)cc2)c2ccc(-c3ccc4oc5ccccc5c4c3)cc2)c(-c2cc3ccccc3c3ccccc23)c1 ; CC1(C)CCC(C)(C)c2cc(-c3ccc(N(c4ccc(-c5ccc6ccccc6c5)cc4)c4ccccc4-c4cc5ccccc5c5ccccc45)cc3)ccc21 ; CC1(C)CCC(C)(C)c2cc(-c3ccc(N(c4ccc(-c5cc6ccccc6c6ccccc56)cc4)c4cccc(-c5cccc6ccccc56)c4)cc3)ccc21]

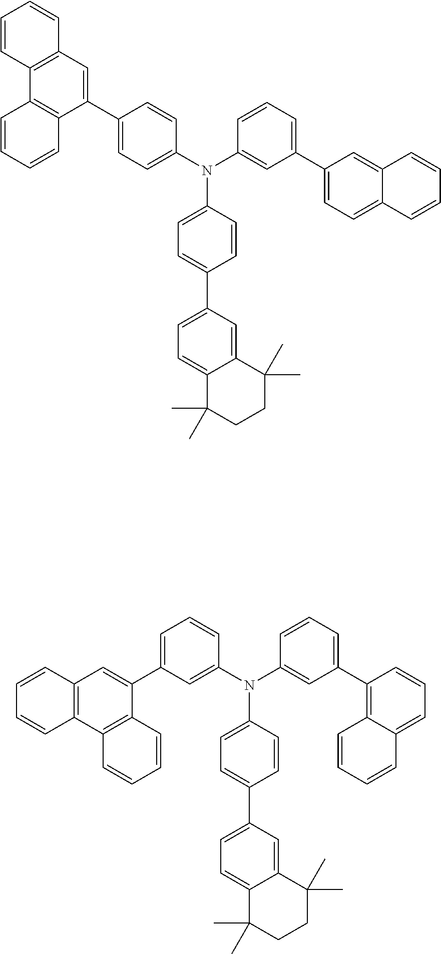 [SMILES: CC1(C)CCC(C)(C)c2cc(-c3ccc(N(c4ccc(-c5cc6ccccc6c6ccccc56)cc4)c4cccc(-c5ccc6ccccc6c5)c4)cc3)ccc21 ; CC1(C)CCC(C)(C)c2cc(-c3ccc(N(c4cccc(-c5cccc6ccccc56)c4)c4cccc(-c5cc6ccccc6c6ccccc56)c4)cc3)ccc21]

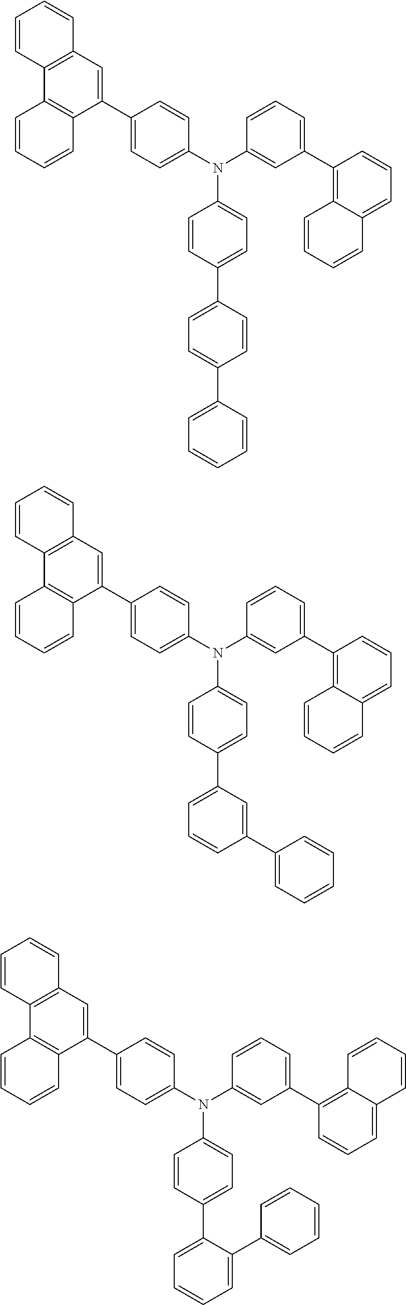 [SMILES: c1ccc(-c2ccc(-c3ccc(N(c4ccc(-c5cc6ccccc6c6ccccc56)cc4)c4cccc(-c5cccc6ccccc56)c4)cc3)cc2)cc1 ; c1ccc(-c2cccc(-c3ccc(N(c4ccc(-c5cc6ccccc6c6ccccc56)cc4)c4cccc(-c5cccc6ccccc56)c4)cc3)c2)cc1 ; c1ccc(-c2ccccc2-c2ccc(N(c3ccc(-c4cc5ccccc5c5ccccc45)cc3)c3cccc(-c4cccc5ccccc45)c3)cc2)cc1]

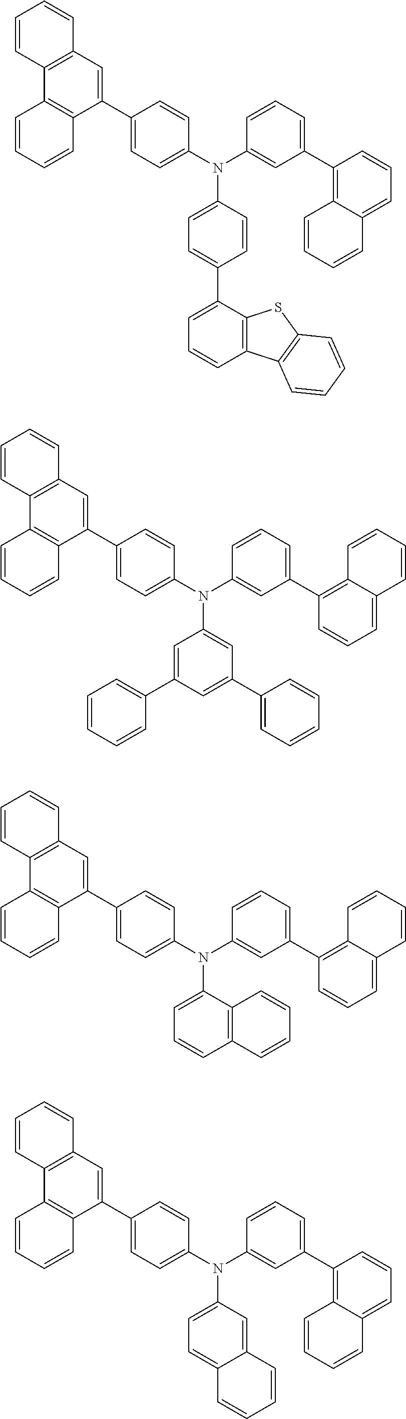 [SMILES: c1cc(-c2cccc3ccccc23)cc(N(c2ccc(-c3cc4ccccc4c4ccccc34)cc2)c2ccc(-c3cccc4c3sc3ccccc34)cc2)c1 ; c1ccc(-c2cc(-c3ccccc3)cc(N(c3ccc(-c4cc5ccccc5c5ccccc45)cc3)c3cccc(-c4cccc5ccccc45)c3)c2)cc1 ; c1cc(-c2cccc3ccccc23)cc(N(c2ccc(-c3cc4ccccc4c4ccccc34)cc2)c2cccc3ccccc23)c1 ; c1cc(-c2cccc3ccccc23)cc(N(c2ccc(-c3cc4ccccc4c4ccccc34)cc2)c2ccc3ccccc3c2)c1]

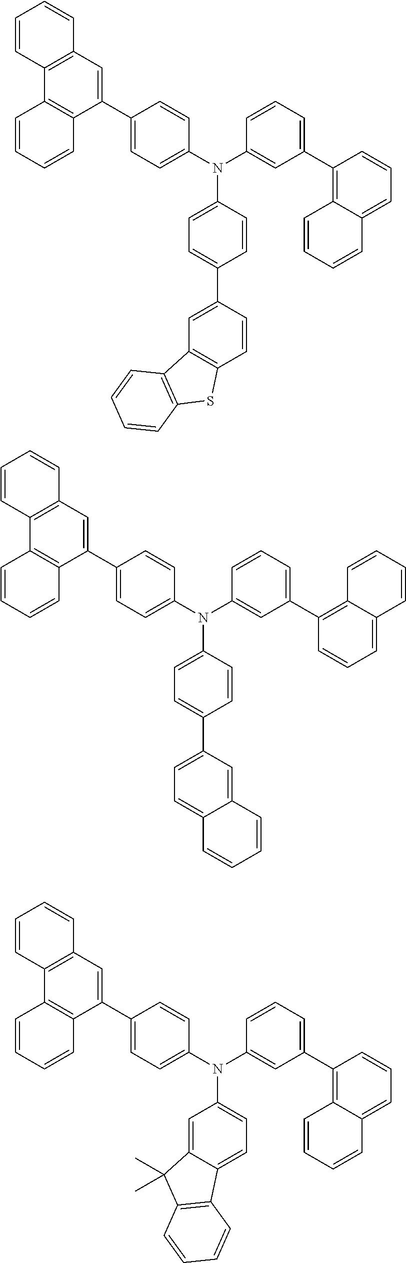 [SMILES: c1cc(-c2cccc3ccccc23)cc(N(c2ccc(-c3ccc4sc5ccccc5c4c3)cc2)c2ccc(-c3cc4ccccc4c4ccccc34)cc2)c1 ; c1cc(-c2cccc3ccccc23)cc(N(c2ccc(-c3ccc4ccccc4c3)cc2)c2ccc(-c3cc4ccccc4c4ccccc34)cc2)c1 ; CC1(C)c2ccccc2-c2ccc(N(c3ccc(-c4cc5ccccc5c5ccccc45)cc3)c3cccc(-c4cccc5ccccc45)c3)cc21]

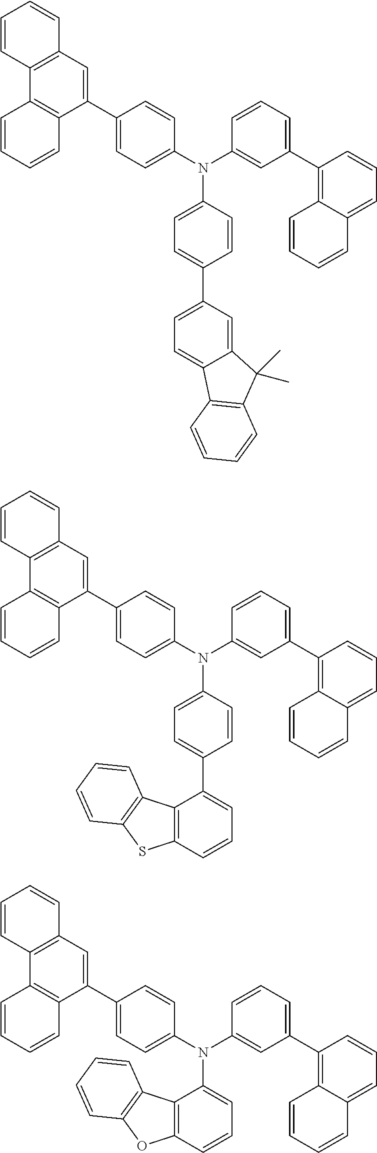 [SMILES: CC1(C)c2ccccc2-c2ccc(-c3ccc(N(c4ccc(-c5cc6ccccc6c6ccccc56)cc4)c4cccc(-c5cccc6ccccc56)c4)cc3)cc21 ; c1cc(-c2cccc3ccccc23)cc(N(c2ccc(-c3cc4ccccc4c4ccccc34)cc2)c2ccc(-c3cccc4sc5ccccc5c34)cc2)c1 ; c1cc(-c2cccc3ccccc23)cc(N(c2ccc(-c3cc4ccccc4c4ccccc34)cc2)c2cccc3oc4ccccc4c23)c1]

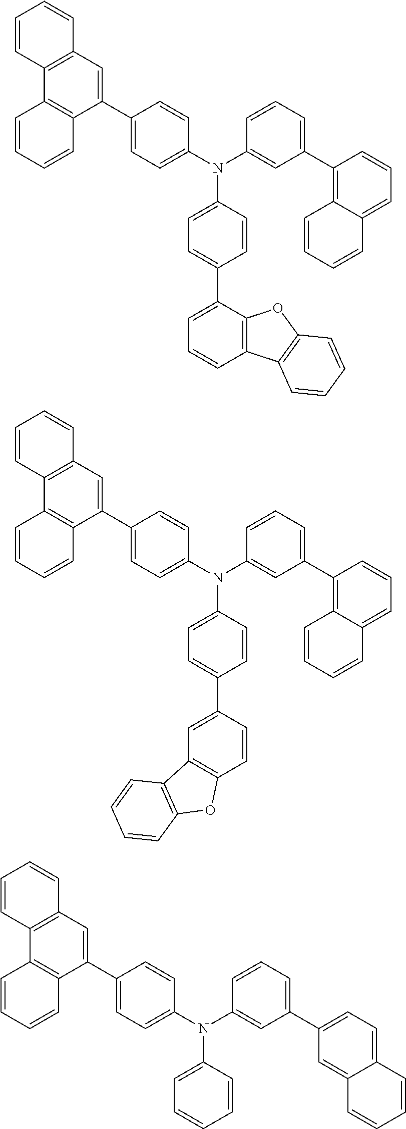 [SMILES: c1cc(-c2cccc3ccccc23)cc(N(c2ccc(-c3cc4ccccc4c4ccccc34)cc2)c2ccc(-c3cccc4c3oc3ccccc34)cc2)c1 ; c1cc(-c2cccc3ccccc23)cc(N(c2ccc(-c3ccc4oc5ccccc5c4c3)cc2)c2ccc(-c3cc4ccccc4c4ccccc34)cc2)c1 ; c1ccc(N(c2ccc(-c3cc4ccccc4c4ccccc34)cc2)c2cccc(-c3ccc4ccccc4c3)c2)cc1]

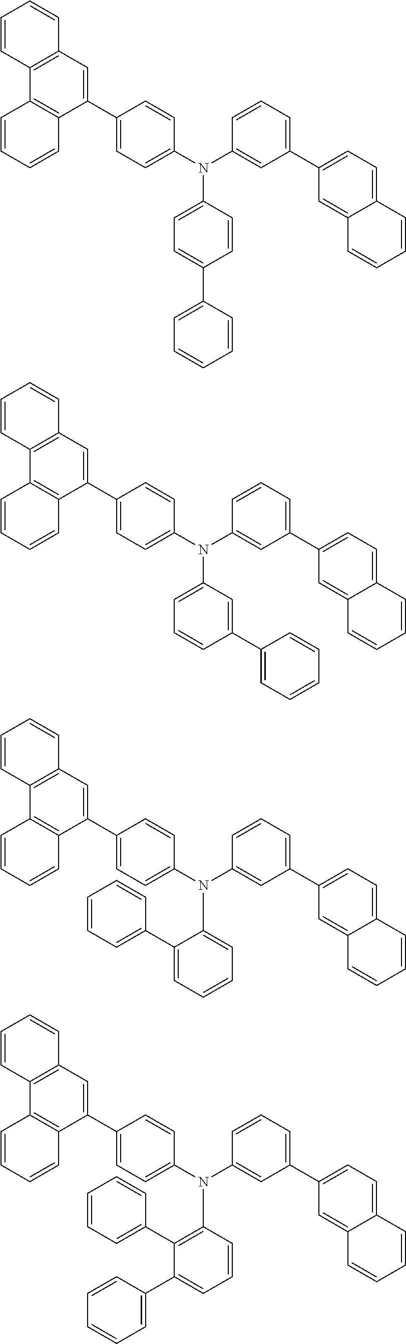 [SMILES: c1ccc(-c2ccc(N(c3ccc(-c4cc5ccccc5c5ccccc45)cc3)c3cccc(-c4ccc5ccccc5c4)c3)cc2)cc1 ; c1ccc(-c2cccc(N(c3ccc(-c4cc5ccccc5c5ccccc45)cc3)c3cccc(-c4ccc5ccccc5c4)c3)c2)cc1 ; c1ccc(-c2ccccc2N(c2ccc(-c3cc4ccccc4c4ccccc34)cc2)c2cccc(-c3ccc4ccccc4c3)c2)cc1 ; c1ccc(-c2cccc(N(c3ccc(-c4cc5ccccc5c5ccccc45)cc3)c3cccc(-c4ccc5ccccc5c4)c3)c2-c2ccccc2)cc1]

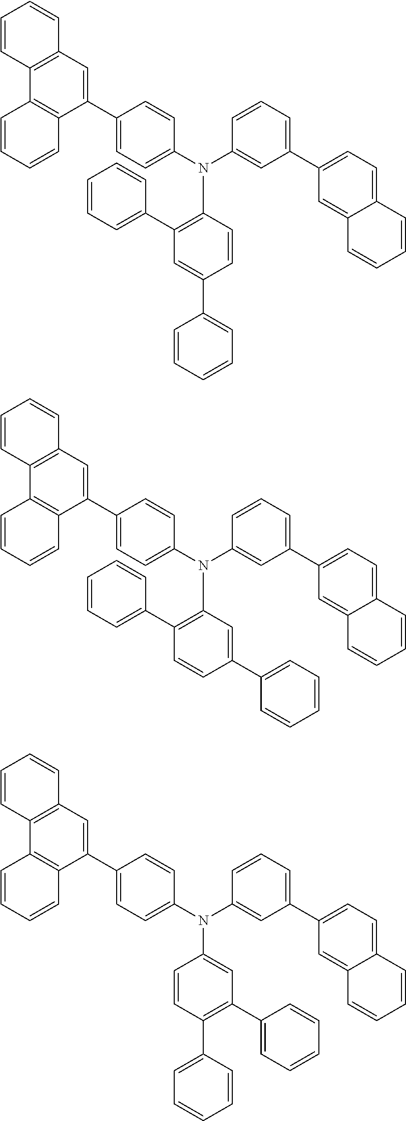 [SMILES: c1ccc(-c2ccc(N(c3ccc(-c4cc5ccccc5c5ccccc45)cc3)c3cccc(-c4ccc5ccccc5c4)c3)c(-c3ccccc3)c2)cc1 ; c1ccc(-c2ccc(-c3ccccc3)c(N(c3ccc(-c4cc5ccccc5c5ccccc45)cc3)c3cccc(-c4ccc5ccccc5c4)c3)c2)cc1 ; c1ccc(-c2ccc(N(c3ccc(-c4cc5ccccc5c5ccccc45)cc3)c3cccc(-c4ccc5ccccc5c4)c3)cc2-c2ccccc2)cc1]

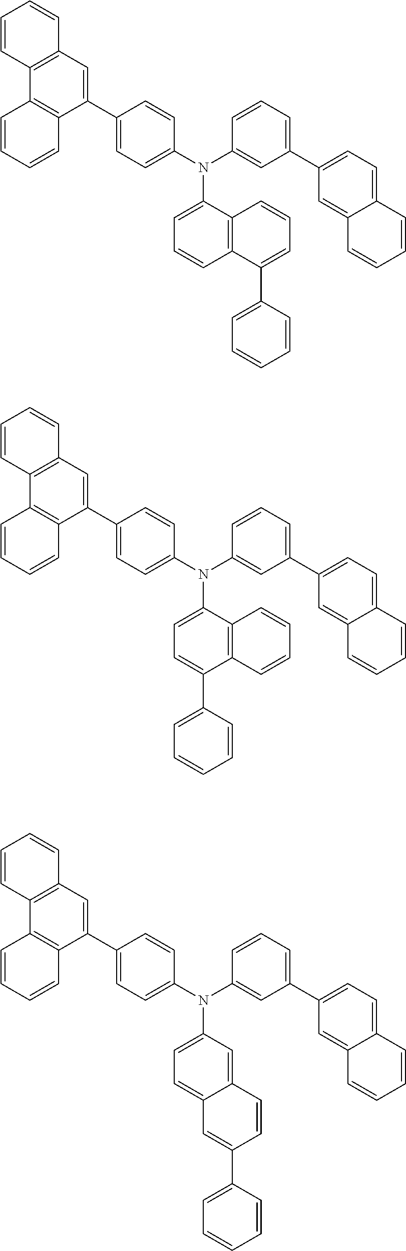 [SMILES: c1ccc(-c2cccc3c(N(c4ccc(-c5cc6ccccc6c6ccccc56)cc4)c4cccc(-c5ccc6ccccc6c5)c4)cccc23)cc1 ; c1ccc(-c2ccc(N(c3ccc(-c4cc5ccccc5c5ccccc45)cc3)c3cccc(-c4ccc5ccccc5c4)c3)c3ccccc23)cc1 ; c1ccc(-c2ccc3cc(N(c4ccc(-c5cc6ccccc6c6ccccc56)cc4)c4cccc(-c5ccc6ccccc6c5)c4)ccc3c2)cc1]

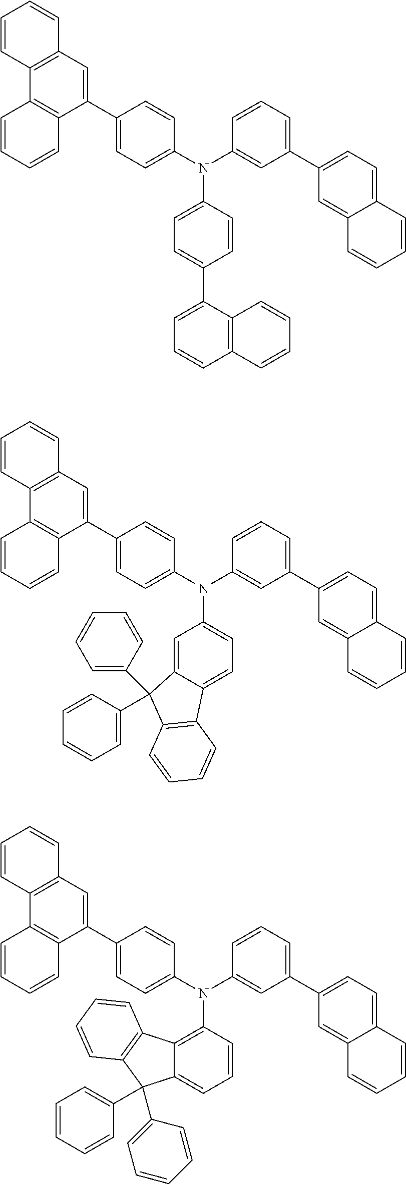 [SMILES: c1cc(-c2ccc3ccccc3c2)cc(N(c2ccc(-c3cccc4ccccc34)cc2)c2ccc(-c3cc4ccccc4c4ccccc34)cc2)c1 ; c1ccc(C2(c3ccccc3)c3ccccc3-c3ccc(N(c4ccc(-c5cc6ccccc6c6ccccc56)cc4)c4cccc(-c5ccc6ccccc6c5)c4)cc32)cc1 ; c1ccc(C2(c3ccccc3)c3ccccc3-c3c(N(c4ccc(-c5cc6ccccc6c6ccccc56)cc4)c4cccc(-c5ccc6ccccc6c5)c4)cccc32)cc1]

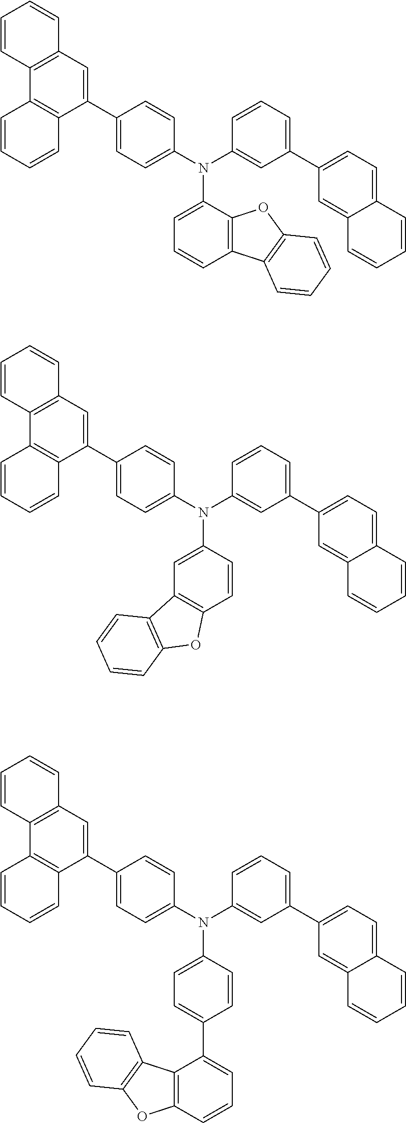 [SMILES: c1cc(-c2ccc3ccccc3c2)cc(N(c2ccc(-c3cc4ccccc4c4ccccc34)cc2)c2cccc3c2oc2ccccc23)c1 ; c1cc(-c2ccc3ccccc3c2)cc(N(c2ccc(-c3cc4ccccc4c4ccccc34)cc2)c2ccc3oc4ccccc4c3c2)c1 ; c1cc(-c2ccc3ccccc3c2)cc(N(c2ccc(-c3cc4ccccc4c4ccccc34)cc2)c2ccc(-c3cccc4oc5ccccc5c34)cc2)c1]

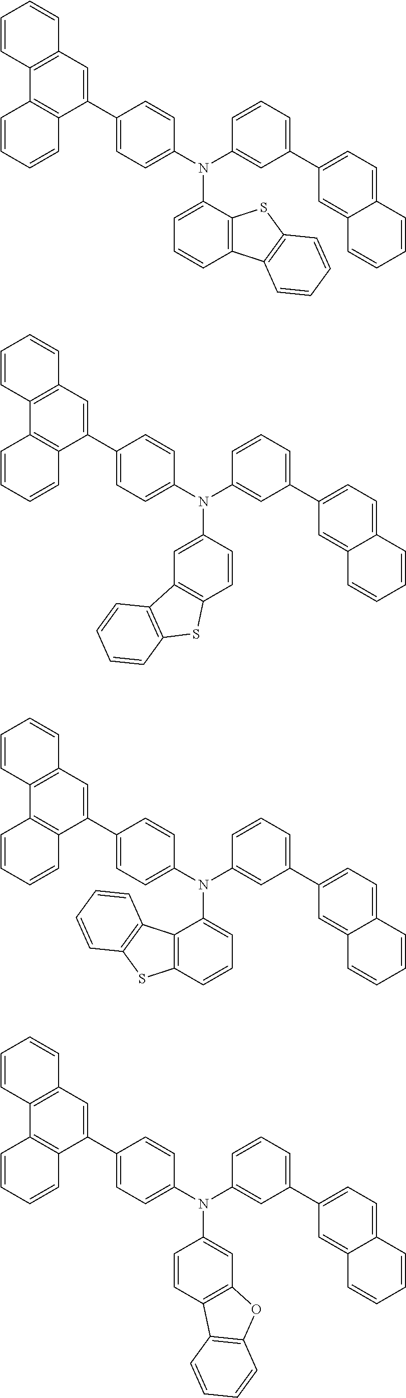 [SMILES: c1cc(-c2ccc3ccccc3c2)cc(N(c2ccc(-c3cc4ccccc4c4ccccc34)cc2)c2cccc3c2sc2ccccc23)c1 ; c1cc(-c2ccc3ccccc3c2)cc(N(c2ccc(-c3cc4ccccc4c4ccccc34)cc2)c2ccc3sc4ccccc4c3c2)c1 ; c1cc(-c2ccc3ccccc3c2)cc(N(c2ccc(-c3cc4ccccc4c4ccccc34)cc2)c2cccc3sc4ccccc4c23)c1 ; c1cc(-c2ccc3ccccc3c2)cc(N(c2ccc(-c3cc4ccccc4c4ccccc34)cc2)c2ccc3c(c2)oc2ccccc23)c1]

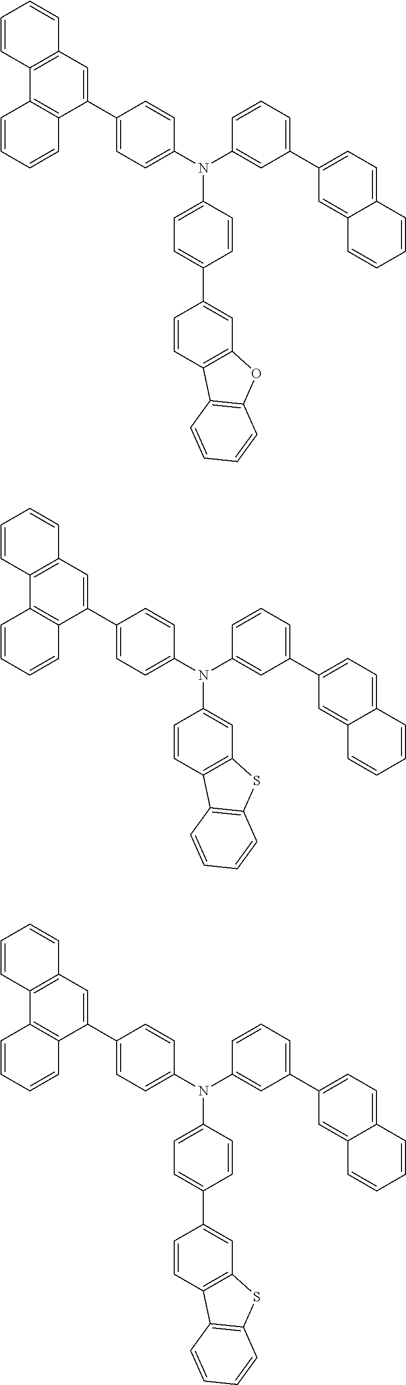 [SMILES: c1cc(-c2ccc3ccccc3c2)cc(N(c2ccc(-c3ccc4c(c3)oc3ccccc34)cc2)c2ccc(-c3cc4ccccc4c4ccccc34)cc2)c1 ; c1cc(-c2ccc3ccccc3c2)cc(N(c2ccc(-c3cc4ccccc4c4ccccc34)cc2)c2ccc3c(c2)sc2ccccc23)c1 ; c1cc(-c2ccc3ccccc3c2)cc(N(c2ccc(-c3ccc4c(c3)sc3ccccc34)cc2)c2ccc(-c3cc4ccccc4c4ccccc34)cc2)c1]

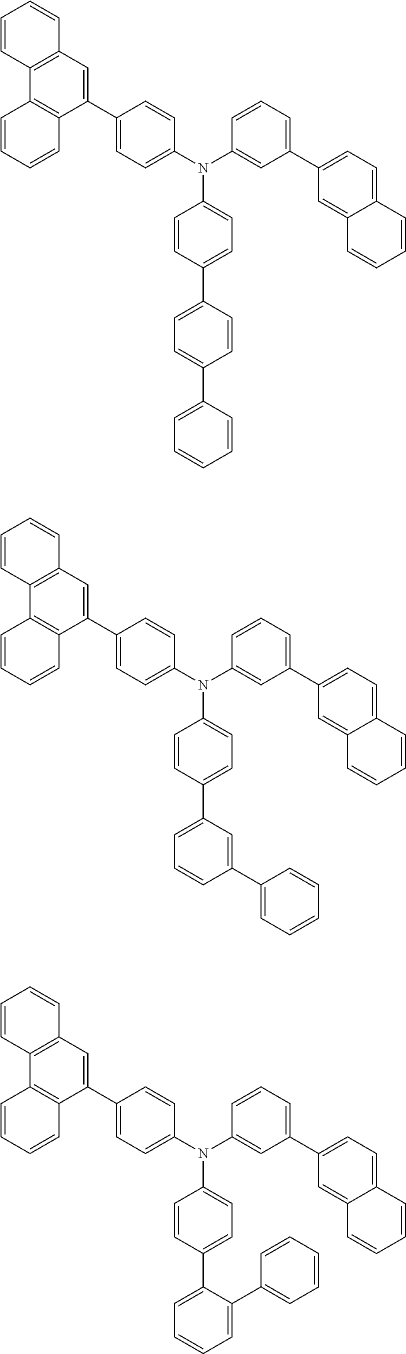 [SMILES: c1ccc(-c2ccc(-c3ccc(N(c4ccc(-c5cc6ccccc6c6ccccc56)cc4)c4cccc(-c5ccc6ccccc6c5)c4)cc3)cc2)cc1 ; c1ccc(-c2cccc(-c3ccc(N(c4ccc(-c5cc6ccccc6c6ccccc56)cc4)c4cccc(-c5ccc6ccccc6c5)c4)cc3)c2)cc1 ; c1ccc(-c2ccccc2-c2ccc(N(c3ccc(-c4cc5ccccc5c5ccccc45)cc3)c3cccc(-c4ccc5ccccc5c4)c3)cc2)cc1]

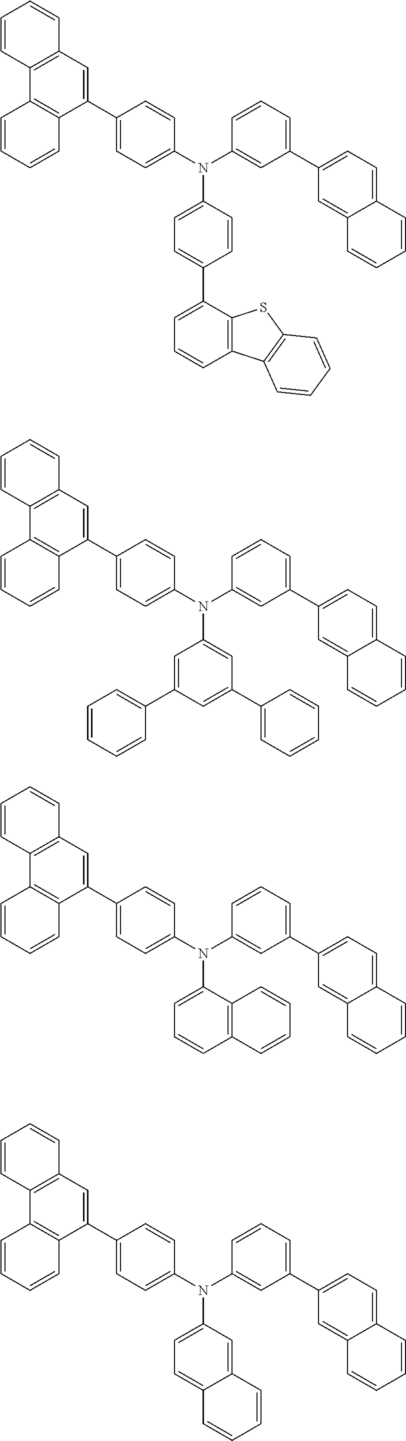 [SMILES: c1cc(-c2ccc3ccccc3c2)cc(N(c2ccc(-c3cc4ccccc4c4ccccc34)cc2)c2ccc(-c3cccc4c3sc3ccccc34)cc2)c1 ; c1ccc(-c2cc(-c3ccccc3)cc(N(c3ccc(-c4cc5ccccc5c5ccccc45)cc3)c3cccc(-c4ccc5ccccc5c4)c3)c2)cc1 ; c1cc(-c2ccc3ccccc3c2)cc(N(c2ccc(-c3cc4ccccc4c4ccccc34)cc2)c2cccc3ccccc23)c1 ; c1cc(-c2ccc3ccccc3c2)cc(N(c2ccc(-c3cc4ccccc4c4ccccc34)cc2)c2ccc3ccccc3c2)c1]

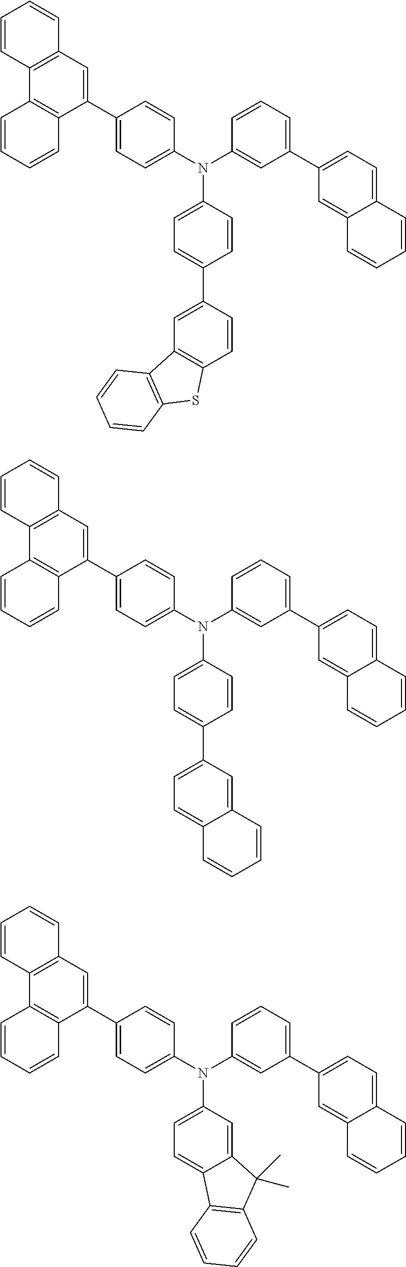 [SMILES: c1cc(-c2ccc3ccccc3c2)cc(N(c2ccc(-c3ccc4sc5ccccc5c4c3)cc2)c2ccc(-c3cc4ccccc4c4ccccc34)cc2)c1 ; c1cc(-c2ccc3ccccc3c2)cc(N(c2ccc(-c3ccc4ccccc4c3)cc2)c2ccc(-c3cc4ccccc4c4ccccc34)cc2)c1 ; CC1(C)c2ccccc2-c2ccc(N(c3ccc(-c4cc5ccccc5c5ccccc45)cc3)c3cccc(-c4ccc5ccccc5c4)c3)cc21]

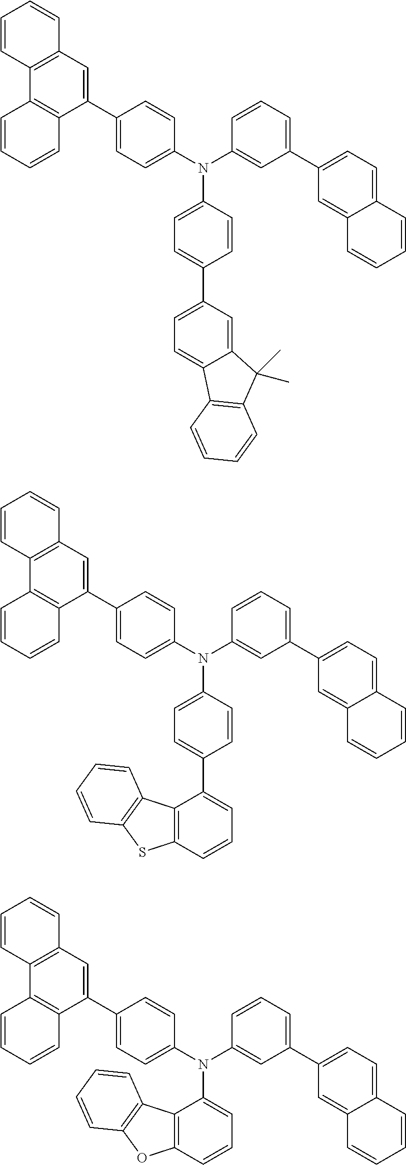 [SMILES: CC1(C)c2ccccc2-c2ccc(-c3ccc(N(c4ccc(-c5cc6ccccc6c6ccccc56)cc4)c4cccc(-c5ccc6ccccc6c5)c4)cc3)cc21 ; c1cc(-c2ccc3ccccc3c2)cc(N(c2ccc(-c3cc4ccccc4c4ccccc34)cc2)c2ccc(-c3cccc4sc5ccccc5c34)cc2)c1 ; c1cc(-c2ccc3ccccc3c2)cc(N(c2ccc(-c3cc4ccccc4c4ccccc34)cc2)c2cccc3oc4ccccc4c23)c1]

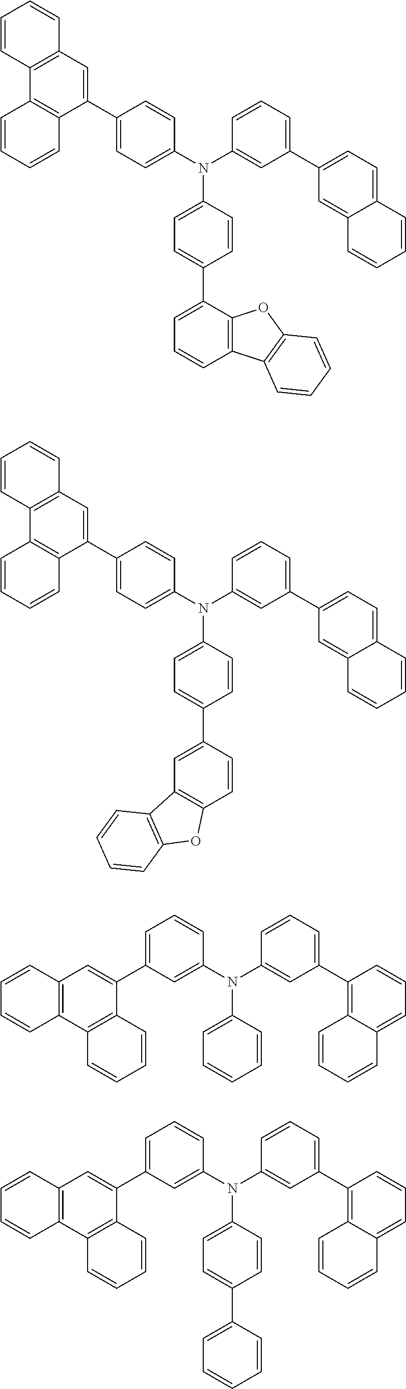 [SMILES: c1cc(-c2ccc3ccccc3c2)cc(N(c2ccc(-c3cc4ccccc4c4ccccc34)cc2)c2ccc(-c3cccc4c3oc3ccccc34)cc2)c1 ; c1cc(-c2ccc3ccccc3c2)cc(N(c2ccc(-c3ccc4oc5ccccc5c4c3)cc2)c2ccc(-c3cc4ccccc4c4ccccc34)cc2)c1 ; c1ccc(N(c2cccc(-c3cccc4ccccc34)c2)c2cccc(-c3cc4ccccc4c4ccccc34)c2)cc1 ; c1ccc(-c2ccc(N(c3cccc(-c4cccc5ccccc45)c3)c3cccc(-c4cc5ccccc5c5ccccc45)c3)cc2)cc1]

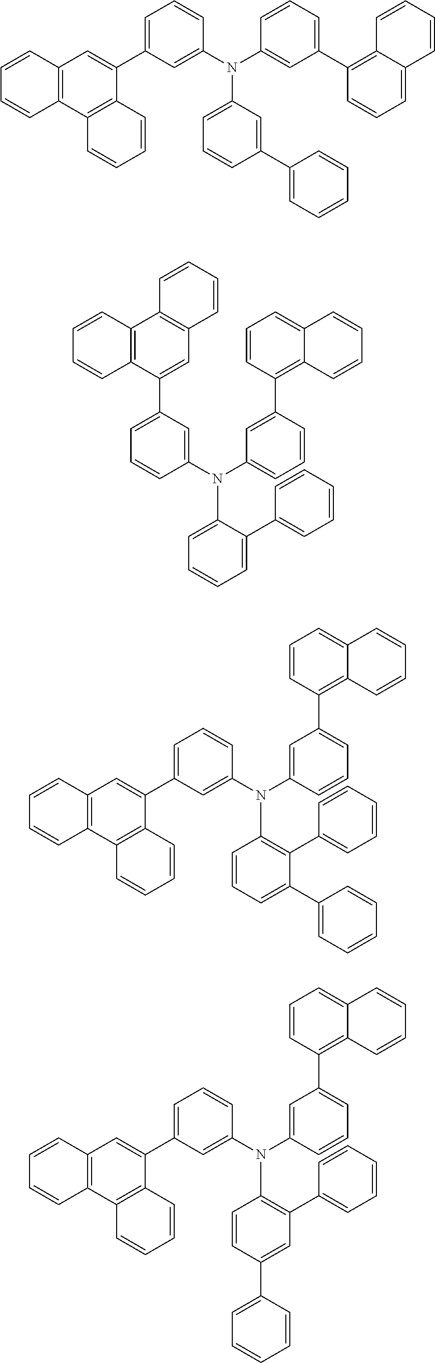

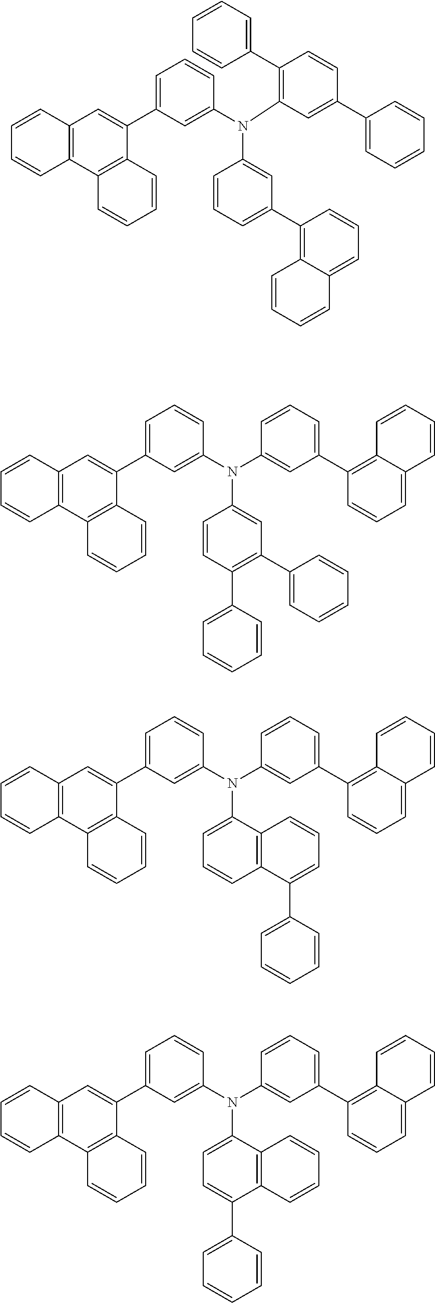 [SMILES: c1ccc(-c2ccc(-c3ccccc3)c(N(c3cccc(-c4cccc5ccccc45)c3)c3cccc(-c4cc5ccccc5c5ccccc45)c3)c2)cc1 ; c1ccc(-c2ccc(N(c3cccc(-c4cccc5ccccc45)c3)c3cccc(-c4cc5ccccc5c5ccccc45)c3)cc2-c2ccccc2)cc1 ; c1ccc(-c2cccc3c(N(c4cccc(-c5cccc6ccccc56)c4)c4cccc(-c5cc6ccccc6c6ccccc56)c4)cccc23)cc1 ; c1ccc(-c2ccc(N(c3cccc(-c4cccc5ccccc45)c3)c3cccc(-c4cc5ccccc5c5ccccc45)c3)c3ccccc23)cc1]

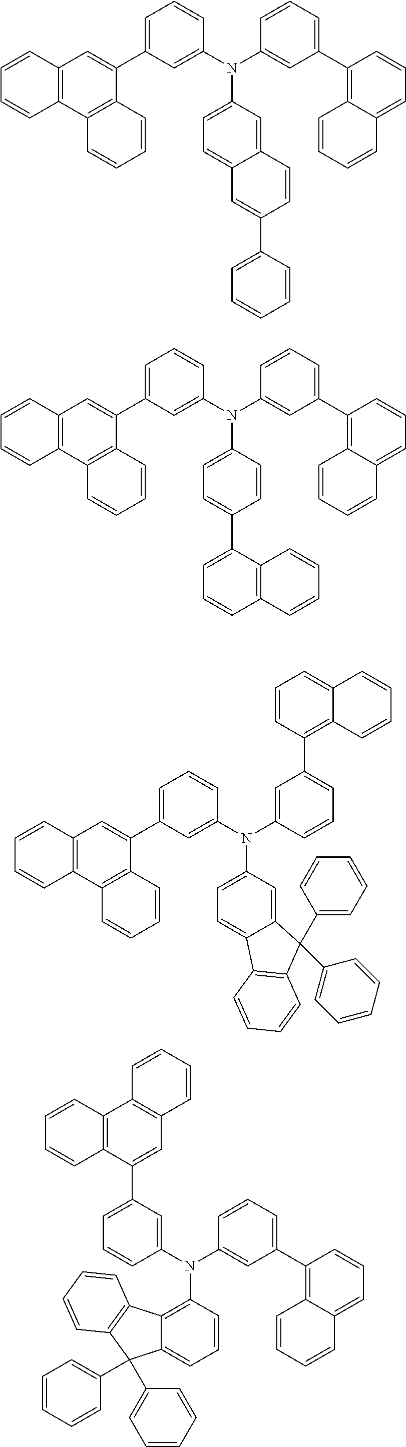

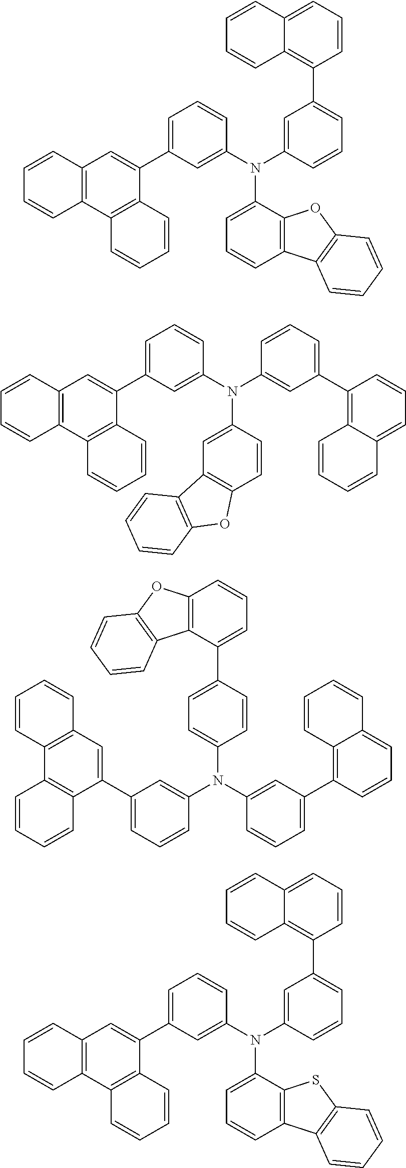

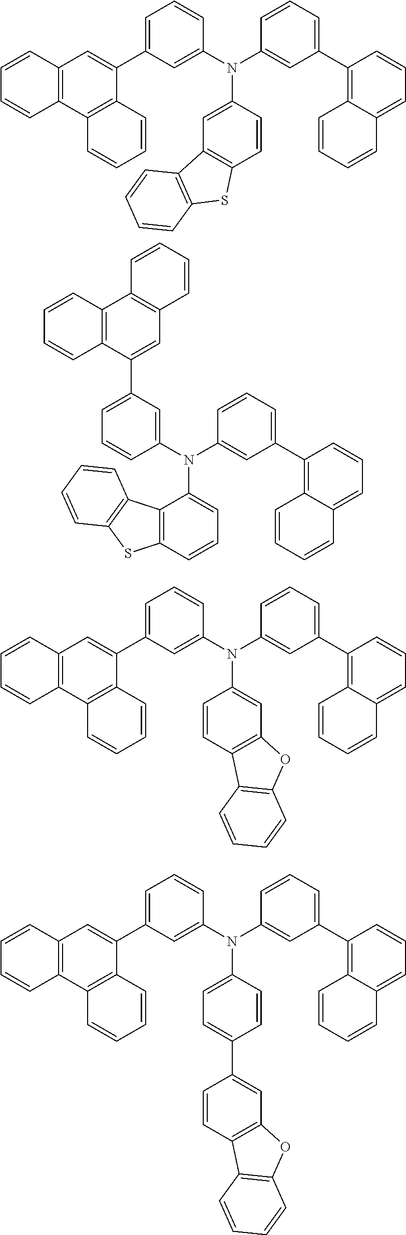

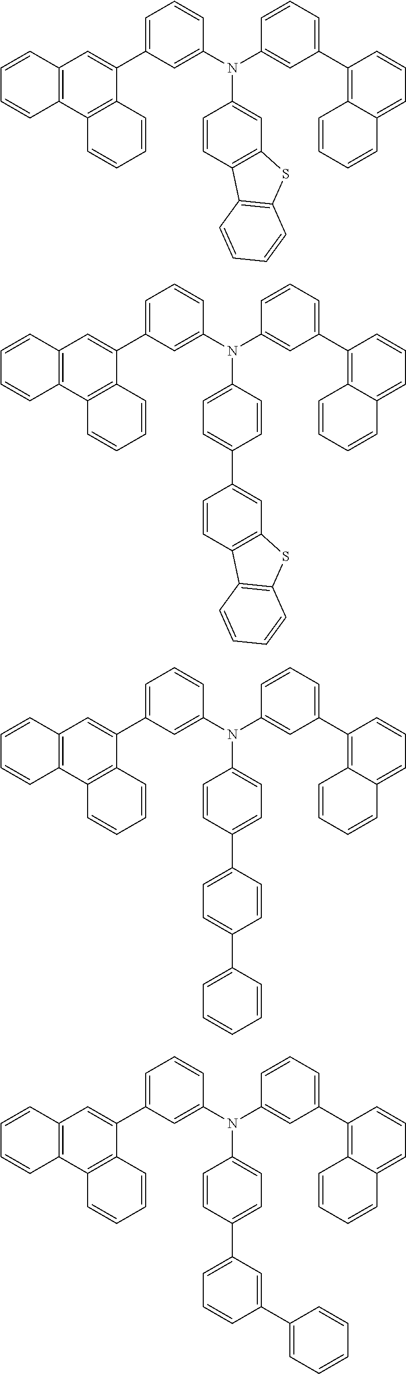

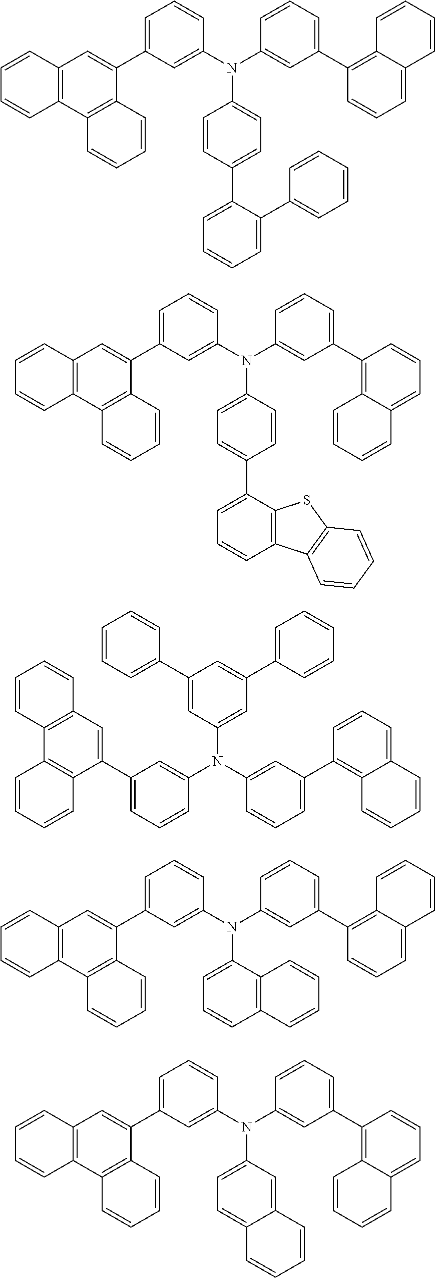

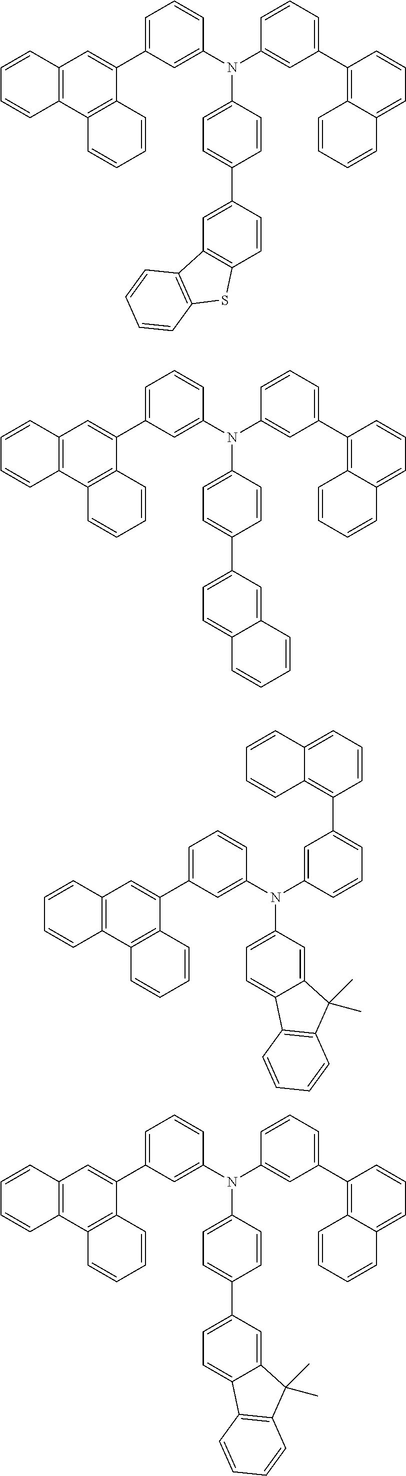

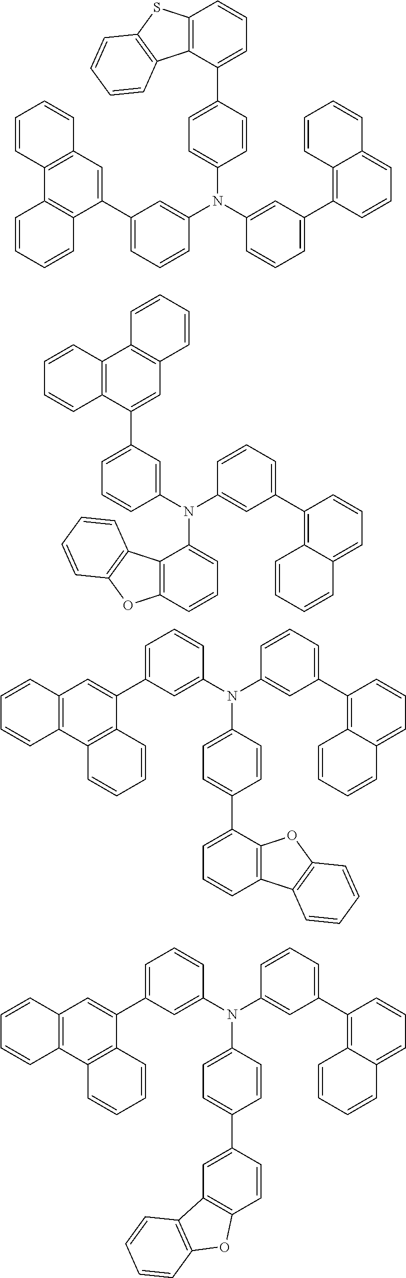

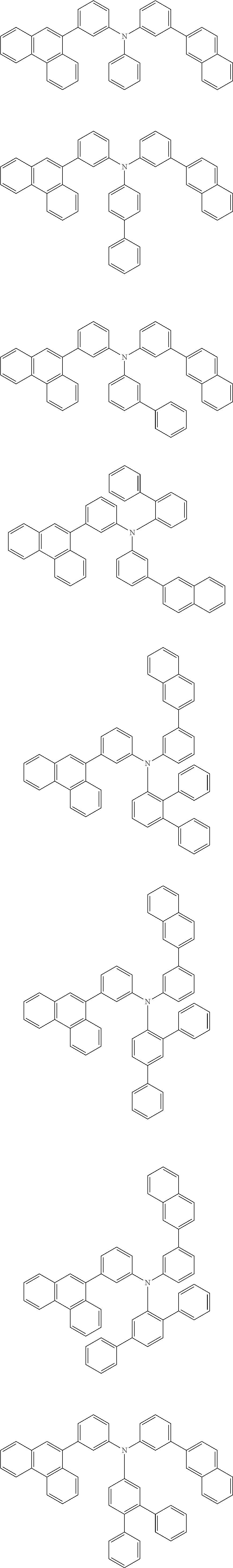

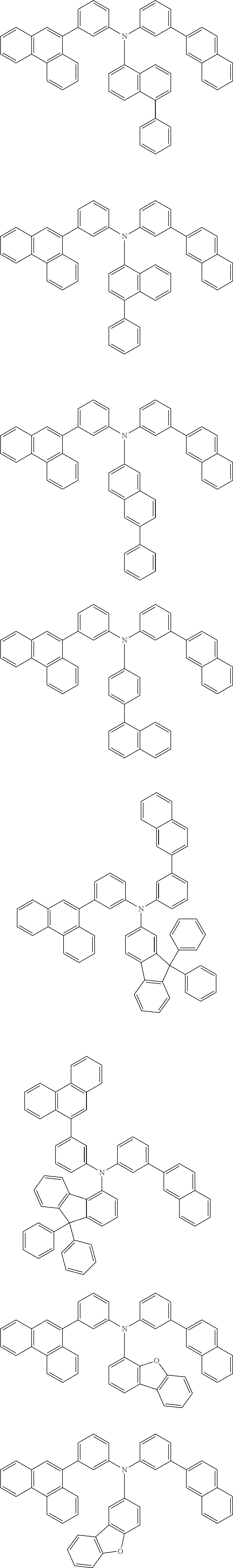

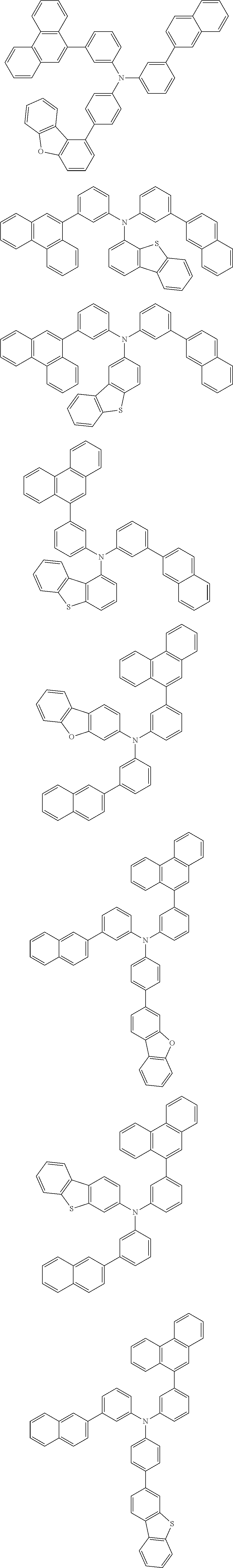

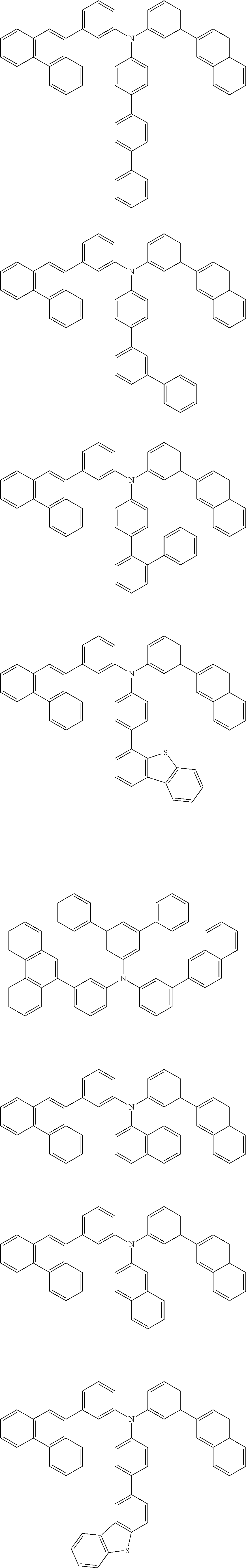

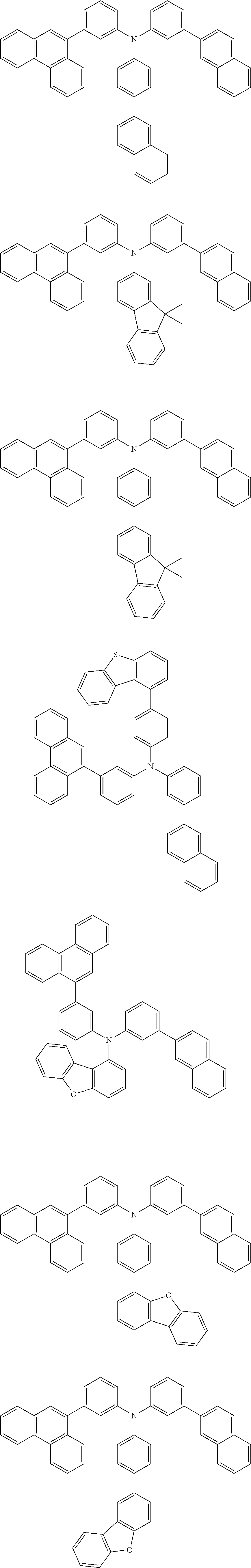

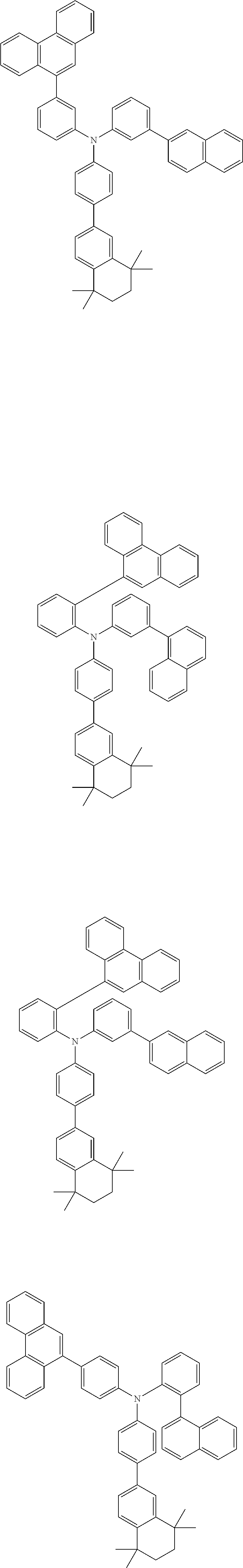 [SMILES: CC1(C)CCC(C)(C)c2cc(-c3ccc(N(c4cccc(-c5ccc6ccccc6c5)c4)c4cccc(-c5cc6ccccc6c6ccccc56)c4)cc3)ccc21 ; CC1(C)CCC(C)(C)c2cc(-c3ccc(N(c4cccc(-c5cccc6ccccc56)c4)c4ccccc4-c4cc5ccccc5c5ccccc45)cc3)ccc21 ; CC1(C)CCC(C)(C)c2cc(-c3ccc(N(c4cccc(-c5ccc6ccccc6c5)c4)c4ccccc4-c4cc5ccccc5c5ccccc45)cc3)ccc21 ; CC1(C)CCC(C)(C)c2cc(-c3ccc(N(c4ccc(-c5cc6ccccc6c6ccccc56)cc4)c4ccccc4-c4cccc5ccccc45)cc3)ccc21]

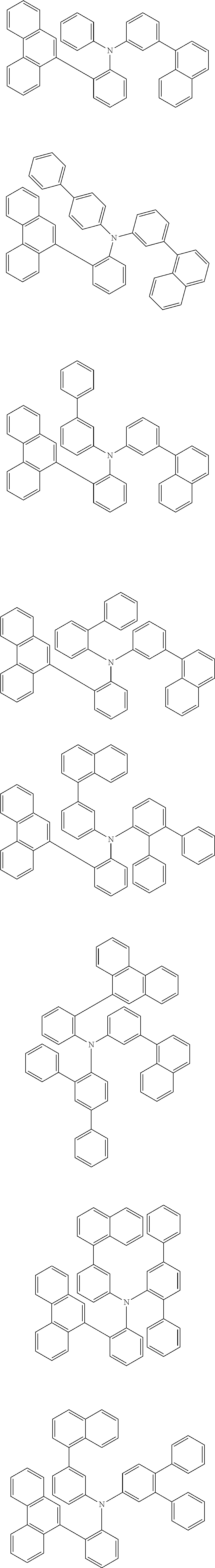

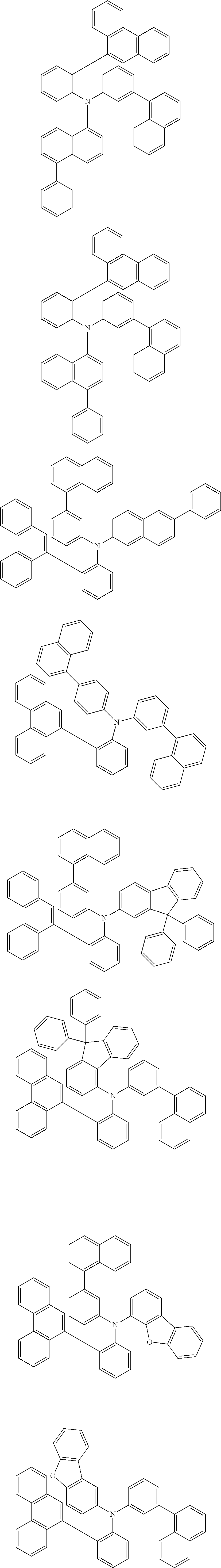

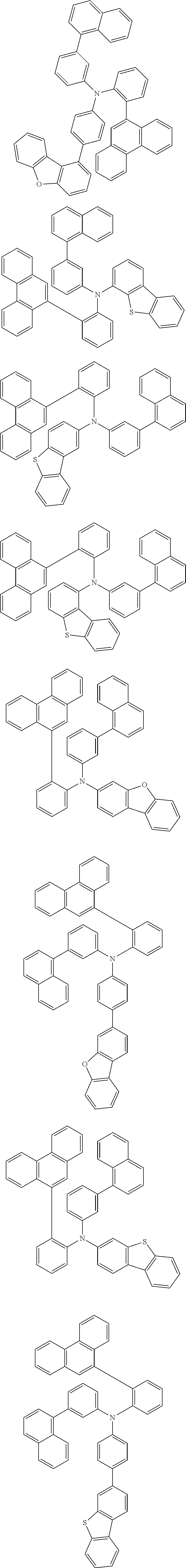

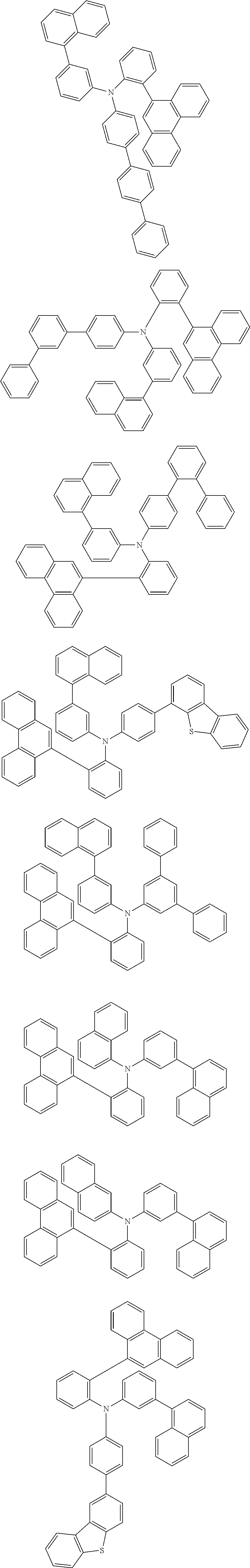

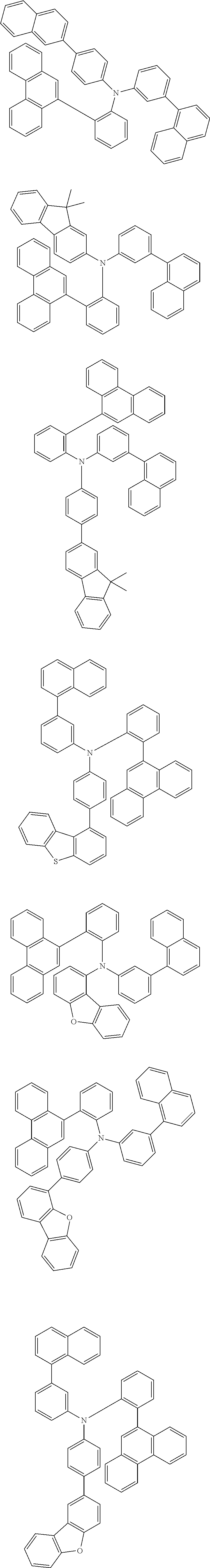

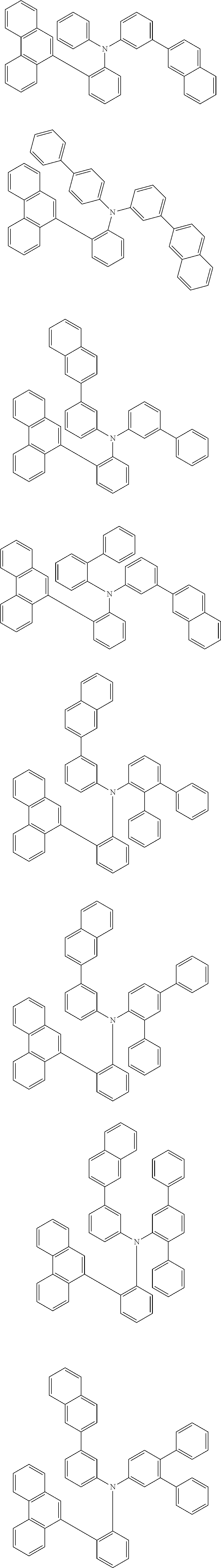

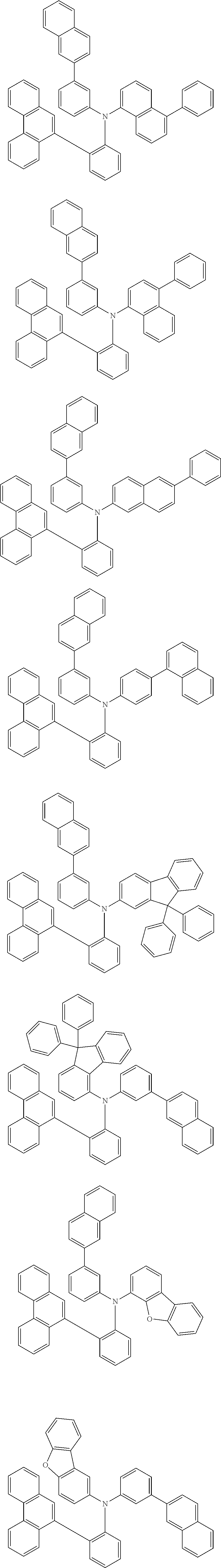

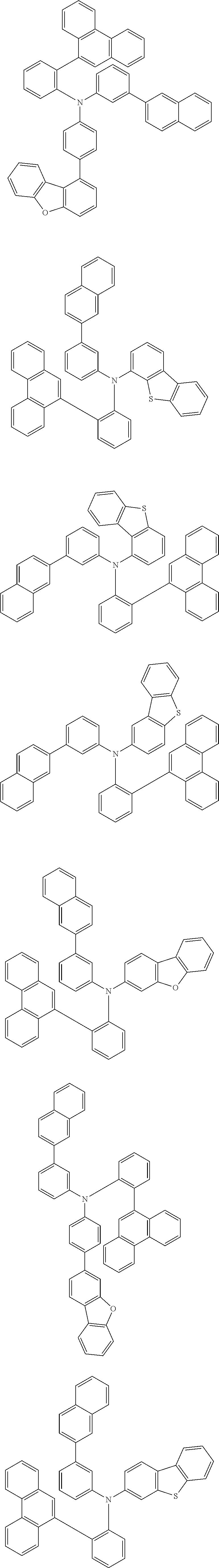

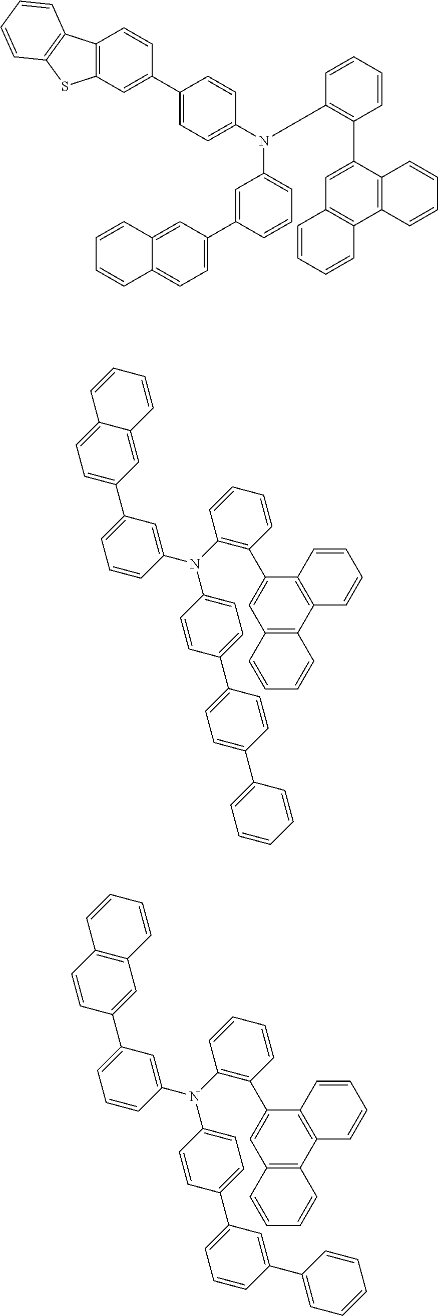 [SMILES: c1cc(-c2ccc3ccccc3c2)cc(N(c2ccc(-c3ccc4c(c3)sc3ccccc34)cc2)c2ccccc2-c2cc3ccccc3c3ccccc23)c1 ; c1ccc(-c2ccc(-c3ccc(N(c4cccc(-c5ccc6ccccc6c5)c4)c4ccccc4-c4cc5ccccc5c5ccccc45)cc3)cc2)cc1 ; c1ccc(-c2cccc(-c3ccc(N(c4cccc(-c5ccc6ccccc6c5)c4)c4ccccc4-c4cc5ccccc5c5ccccc45)cc3)c2)cc1]

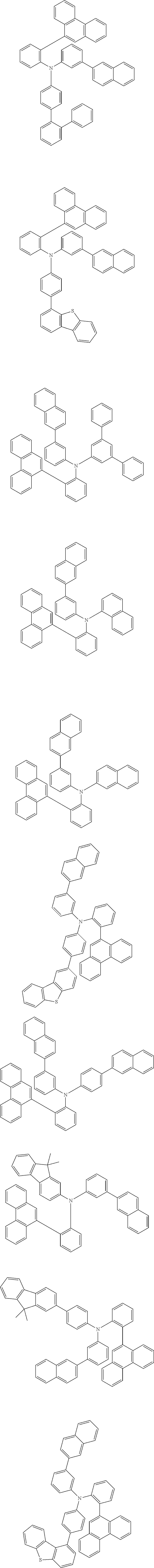

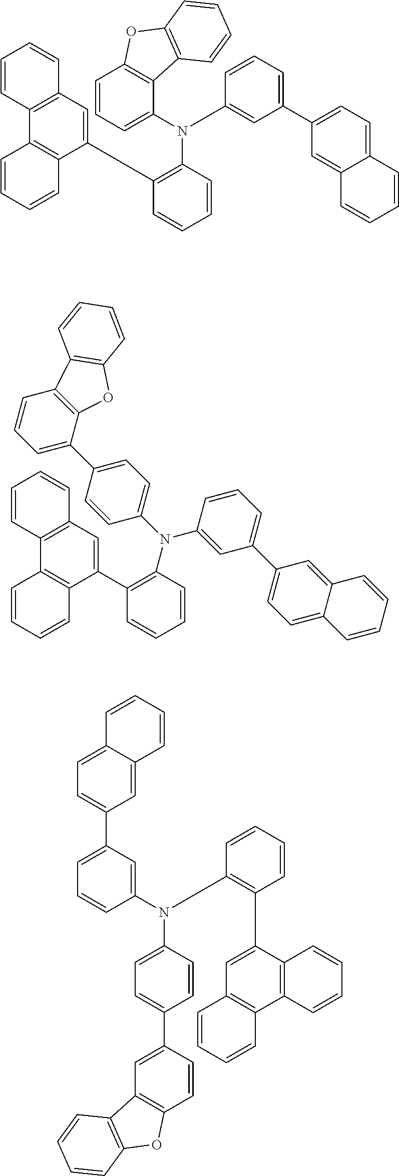 [SMILES: c1cc(-c2ccc3ccccc3c2)cc(N(c2ccccc2-c2cc3ccccc3c3ccccc23)c2cccc3oc4ccccc4c23)c1 ; c1cc(-c2ccc3ccccc3c2)cc(N(c2ccc(-c3cccc4c3oc3ccccc34)cc2)c2ccccc2-c2cc3ccccc3c3ccccc23)c1 ; c1cc(-c2ccc3ccccc3c2)cc(N(c2ccc(-c3ccc4oc5ccccc5c4c3)cc2)c2ccccc2-c2cc3ccccc3c3ccccc23)c1]

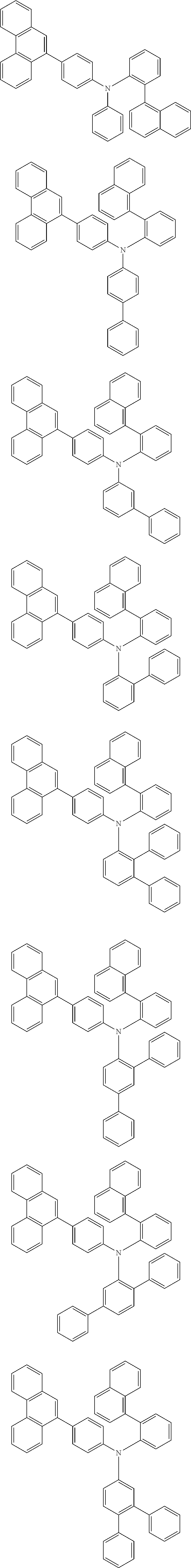

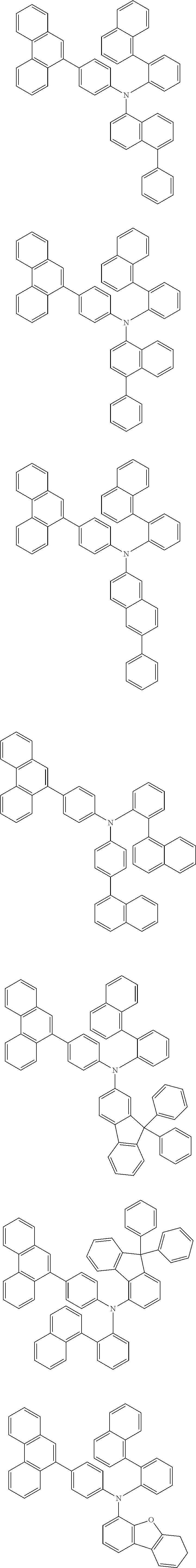

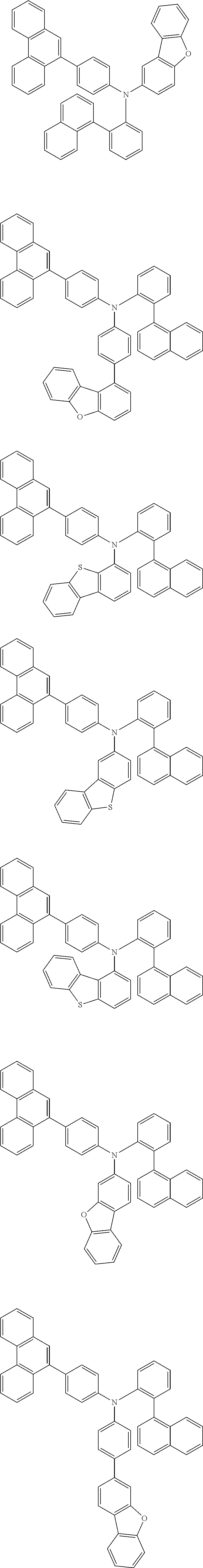

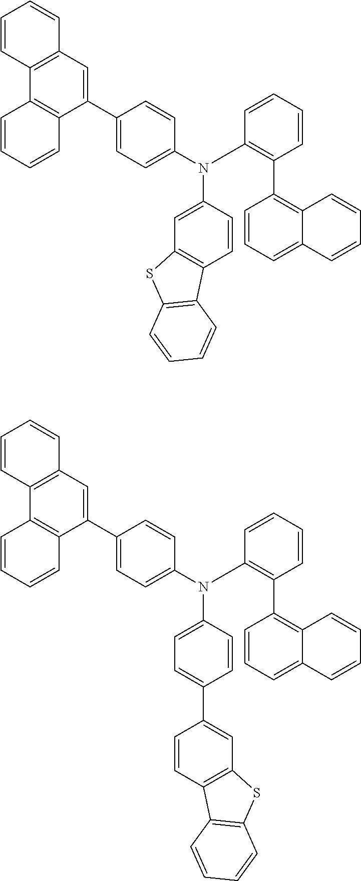 [SMILES: c1ccc(N(c2ccc(-c3cc4ccccc4c4ccccc34)cc2)c2ccc3c(c2)sc2ccccc23)c(-c2cccc3ccccc23)c1 ; c1ccc(N(c2ccc(-c3ccc4c(c3)sc3ccccc34)cc2)c2ccc(-c3cc4ccccc4c4ccccc34)cc2)c(-c2cccc3ccccc23)c1]

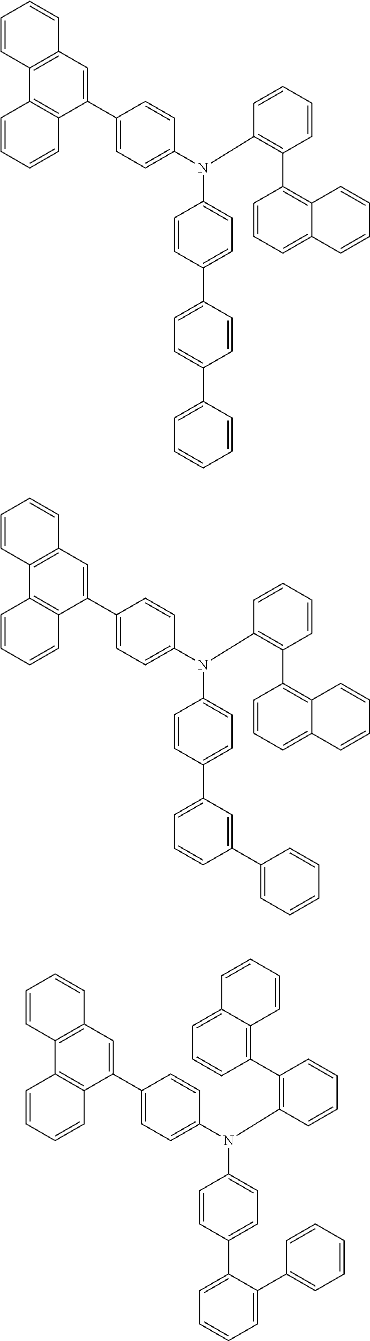 [SMILES: c1ccc(-c2ccc(-c3ccc(N(c4ccc(-c5cc6ccccc6c6ccccc56)cc4)c4ccccc4-c4cccc5ccccc45)cc3)cc2)cc1 ; c1ccc(-c2cccc(-c3ccc(N(c4ccc(-c5cc6ccccc6c6ccccc56)cc4)c4ccccc4-c4cccc5ccccc45)cc3)c2)cc1 ; c1ccc(-c2ccccc2-c2ccc(N(c3ccc(-c4cc5ccccc5c5ccccc45)cc3)c3ccccc3-c3cccc4ccccc34)cc2)cc1]

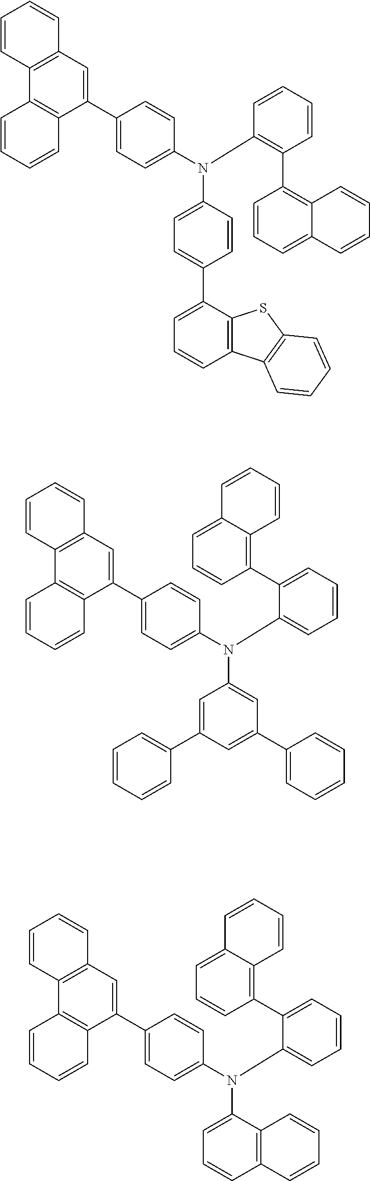 [SMILES: c1ccc(N(c2ccc(-c3cc4ccccc4c4ccccc34)cc2)c2ccc(-c3cccc4c3sc3ccccc34)cc2)c(-c2cccc3ccccc23)c1 ; c1ccc(-c2cc(-c3ccccc3)cc(N(c3ccc(-c4cc5ccccc5c5ccccc45)cc3)c3ccccc3-c3cccc4ccccc34)c2)cc1 ; c1ccc(N(c2ccc(-c3cc4ccccc4c4ccccc34)cc2)c2cccc3ccccc23)c(-c2cccc3ccccc23)c1]

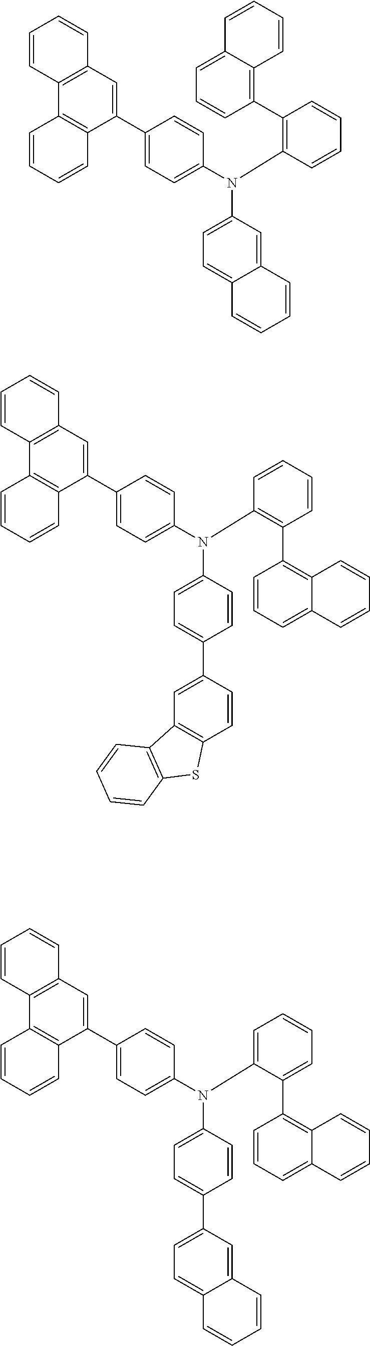 [SMILES: c1ccc(N(c2ccc(-c3cc4ccccc4c4ccccc34)cc2)c2ccc3ccccc3c2)c(-c2cccc3ccccc23)c1 ; c1ccc(N(c2ccc(-c3ccc4sc5ccccc5c4c3)cc2)c2ccc(-c3cc4ccccc4c4ccccc34)cc2)c(-c2cccc3ccccc23)c1 ; c1ccc(N(c2ccc(-c3ccc4ccccc4c3)cc2)c2ccc(-c3cc4ccccc4c4ccccc34)cc2)c(-c2cccc3ccccc23)c1]

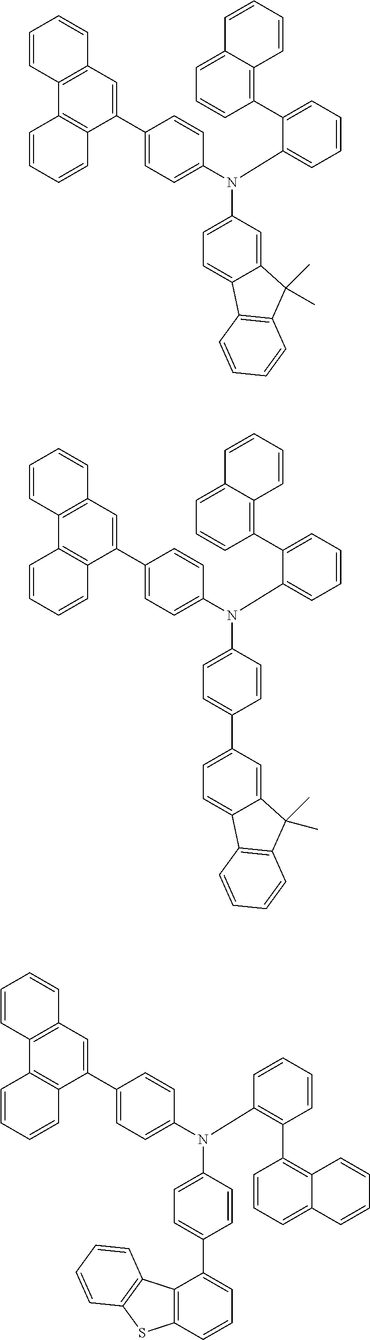 [SMILES: CC1(C)c2ccccc2-c2ccc(N(c3ccc(-c4cc5ccccc5c5ccccc45)cc3)c3ccccc3-c3cccc4ccccc34)cc21 ; CC1(C)c2ccccc2-c2ccc(-c3ccc(N(c4ccc(-c5cc6ccccc6c6ccccc56)cc4)c4ccccc4-c4cccc5ccccc45)cc3)cc21 ; c1ccc(N(c2ccc(-c3cc4ccccc4c4ccccc34)cc2)c2ccc(-c3cccc4sc5ccccc5c34)cc2)c(-c2cccc3ccccc23)c1]

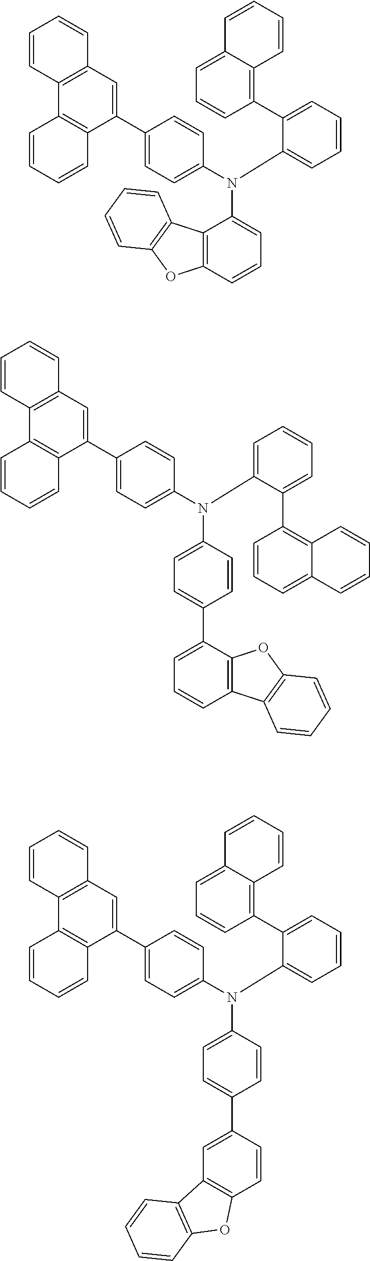 [SMILES: c1ccc(N(c2ccc(-c3cc4ccccc4c4ccccc34)cc2)c2cccc3oc4ccccc4c23)c(-c2cccc3ccccc23)c1 ; c1ccc(N(c2ccc(-c3cc4ccccc4c4ccccc34)cc2)c2ccc(-c3cccc4c3oc3ccccc34)cc2)c(-c2cccc3ccccc23)c1 ; c1ccc(N(c2ccc(-c3ccc4oc5ccccc5c4c3)cc2)c2ccc(-c3cc4ccccc4c4ccccc34)cc2)c(-c2cccc3ccccc23)c1]

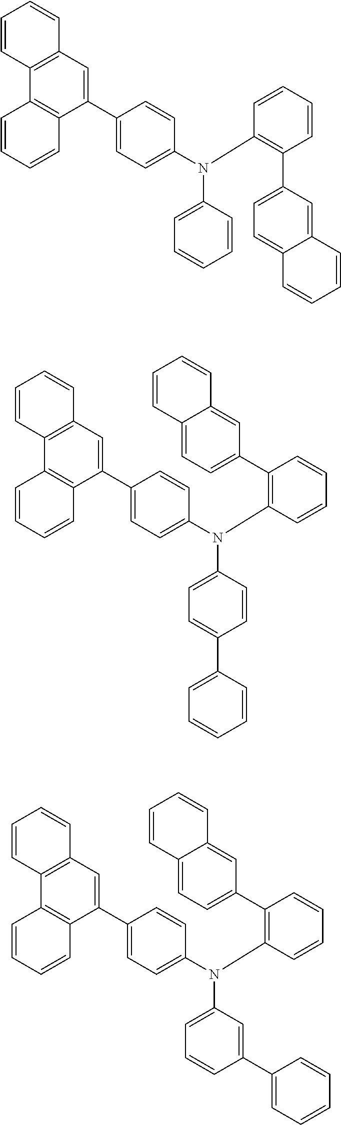 [SMILES: c1ccc(N(c2ccc(-c3cc4ccccc4c4ccccc34)cc2)c2ccccc2-c2ccc3ccccc3c2)cc1 ; c1ccc(-c2ccc(N(c3ccc(-c4cc5ccccc5c5ccccc45)cc3)c3ccccc3-c3ccc4ccccc4c3)cc2)cc1 ; c1ccc(-c2cccc(N(c3ccc(-c4cc5ccccc5c5ccccc45)cc3)c3ccccc3-c3ccc4ccccc4c3)c2)cc1]

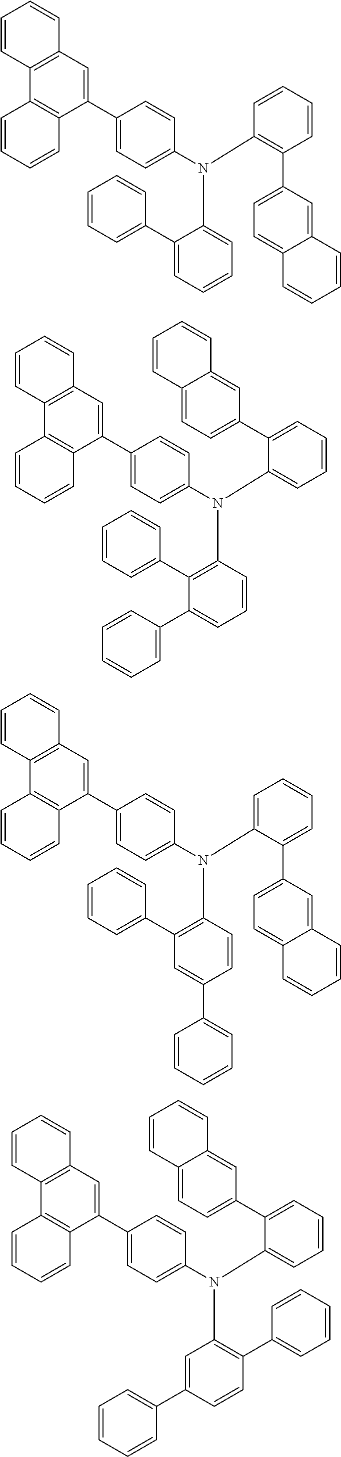

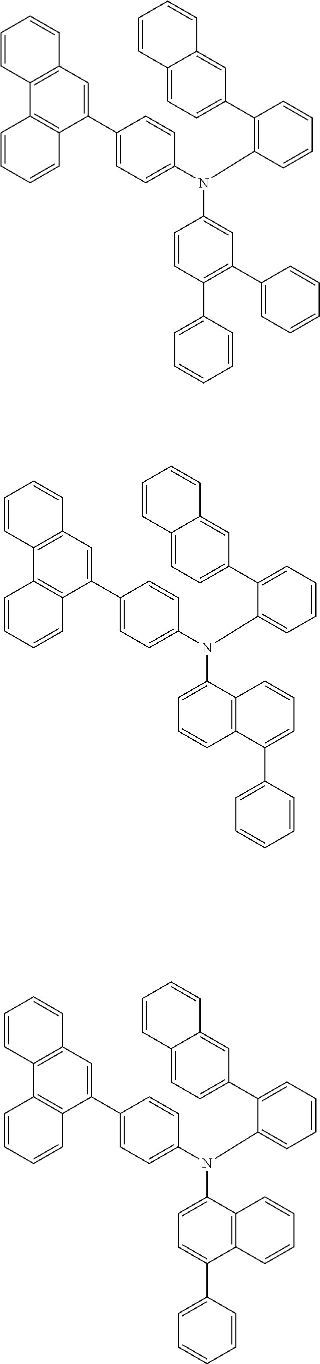 [SMILES: c1ccc(-c2ccc(N(c3ccc(-c4cc5ccccc5c5ccccc45)cc3)c3ccccc3-c3ccc4ccccc4c3)cc2-c2ccccc2)cc1 ; c1ccc(-c2cccc3c(N(c4ccc(-c5cc6ccccc6c6ccccc56)cc4)c4ccccc4-c4ccc5ccccc5c4)cccc23)cc1 ; c1ccc(-c2ccc(N(c3ccc(-c4cc5ccccc5c5ccccc45)cc3)c3ccccc3-c3ccc4ccccc4c3)c3ccccc23)cc1]

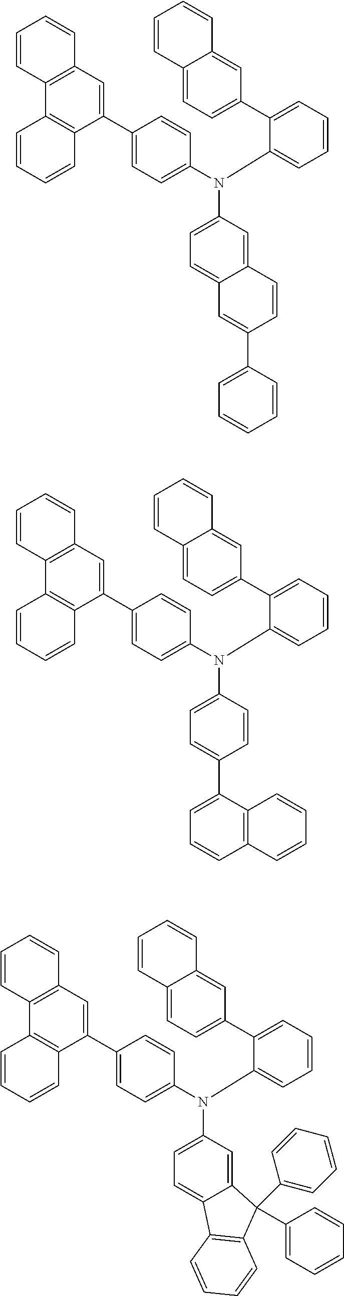 [SMILES: c1ccc(-c2ccc3cc(N(c4ccc(-c5cc6ccccc6c6ccccc56)cc4)c4ccccc4-c4ccc5ccccc5c4)ccc3c2)cc1 ; c1ccc(N(c2ccc(-c3cccc4ccccc34)cc2)c2ccc(-c3cc4ccccc4c4ccccc34)cc2)c(-c2ccc3ccccc3c2)c1 ; c1ccc(C2(c3ccccc3)c3ccccc3-c3ccc(N(c4ccc(-c5cc6ccccc6c6ccccc56)cc4)c4ccccc4-c4ccc5ccccc5c4)cc32)cc1]

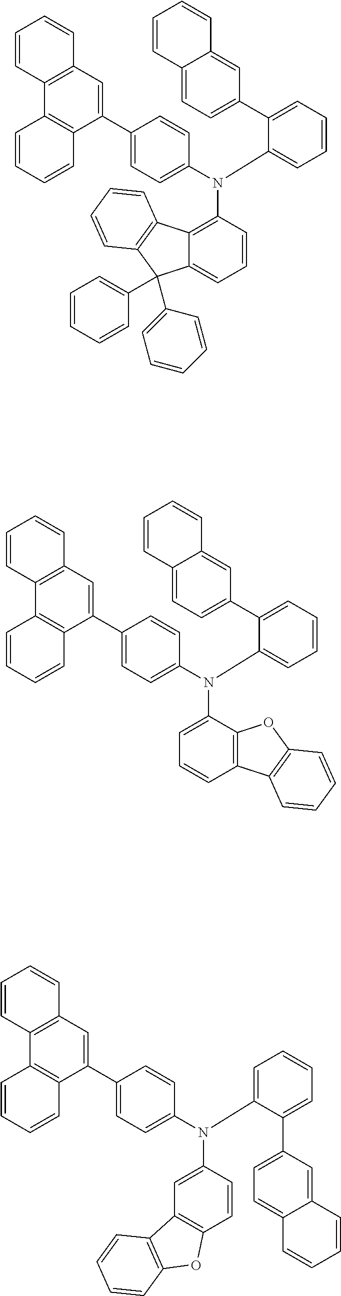 [SMILES: c1ccc(C2(c3ccccc3)c3ccccc3-c3c(N(c4ccc(-c5cc6ccccc6c6ccccc56)cc4)c4ccccc4-c4ccc5ccccc5c4)cccc32)cc1 ; c1ccc(N(c2ccc(-c3cc4ccccc4c4ccccc34)cc2)c2cccc3c2oc2ccccc23)c(-c2ccc3ccccc3c2)c1 ; c1ccc(N(c2ccc(-c3cc4ccccc4c4ccccc34)cc2)c2ccc3oc4ccccc4c3c2)c(-c2ccc3ccccc3c2)c1]

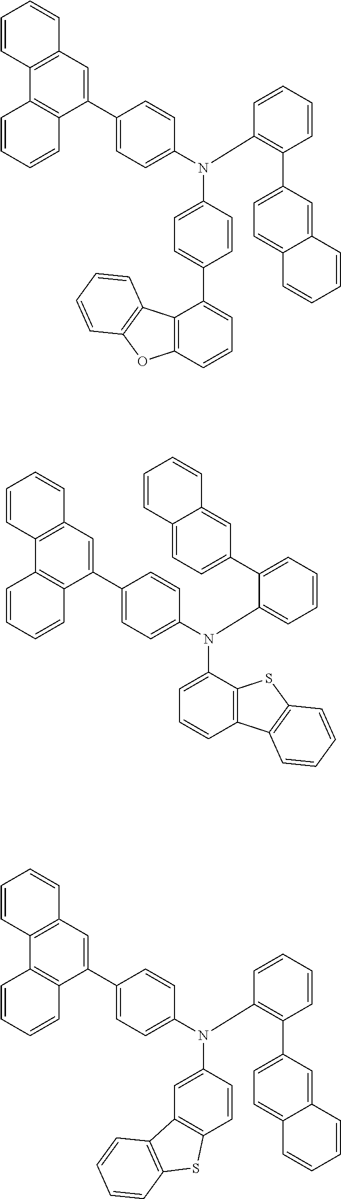 [SMILES: c1ccc(N(c2ccc(-c3cc4ccccc4c4ccccc34)cc2)c2ccc(-c3cccc4oc5ccccc5c34)cc2)c(-c2ccc3ccccc3c2)c1 ; c1ccc(N(c2ccc(-c3cc4ccccc4c4ccccc34)cc2)c2cccc3c2sc2ccccc23)c(-c2ccc3ccccc3c2)c1 ; c1ccc(N(c2ccc(-c3cc4ccccc4c4ccccc34)cc2)c2ccc3sc4ccccc4c3c2)c(-c2ccc3ccccc3c2)c1]

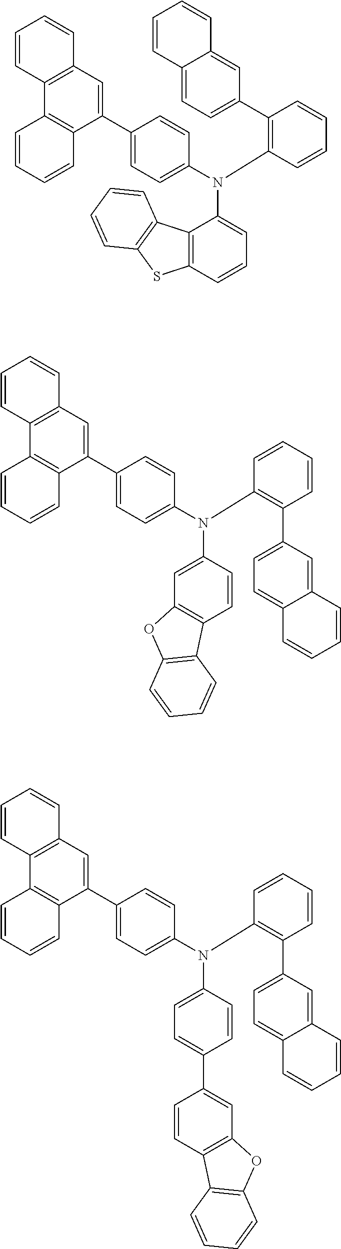 [SMILES: c1ccc(N(c2ccc(-c3cc4ccccc4c4ccccc34)cc2)c2cccc3sc4ccccc4c23)c(-c2ccc3ccccc3c2)c1 ; c1ccc(N(c2ccc(-c3cc4ccccc4c4ccccc34)cc2)c2ccc3c(c2)oc2ccccc23)c(-c2ccc3ccccc3c2)c1 ; c1ccc(N(c2ccc(-c3ccc4c(c3)oc3ccccc34)cc2)c2ccc(-c3cc4ccccc4c4ccccc34)cc2)c(-c2ccc3ccccc3c2)c1]

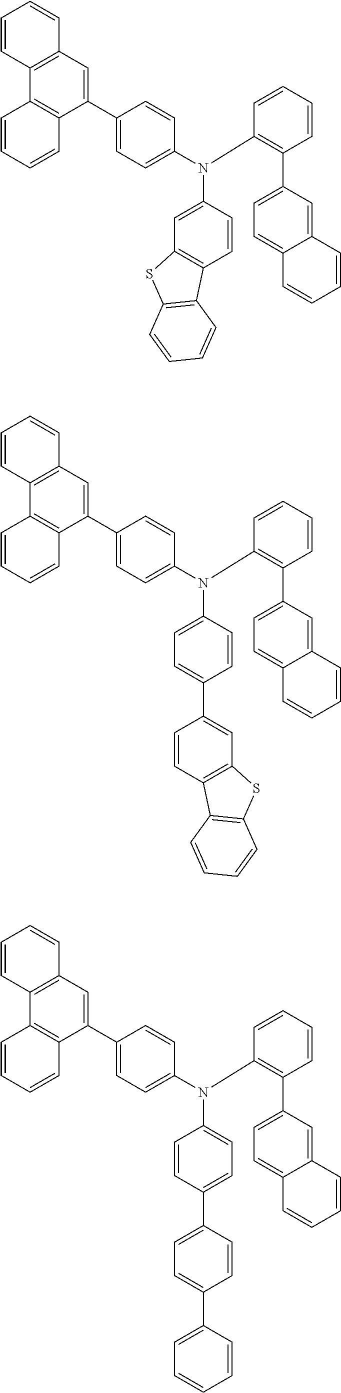 [SMILES: c1ccc(N(c2ccc(-c3cc4ccccc4c4ccccc34)cc2)c2ccc3c(c2)sc2ccccc23)c(-c2ccc3ccccc3c2)c1 ; c1ccc(N(c2ccc(-c3ccc4c(c3)sc3ccccc34)cc2)c2ccc(-c3cc4ccccc4c4ccccc34)cc2)c(-c2ccc3ccccc3c2)c1 ; c1ccc(-c2ccc(-c3ccc(N(c4ccc(-c5cc6ccccc6c6ccccc56)cc4)c4ccccc4-c4ccc5ccccc5c4)cc3)cc2)cc1]

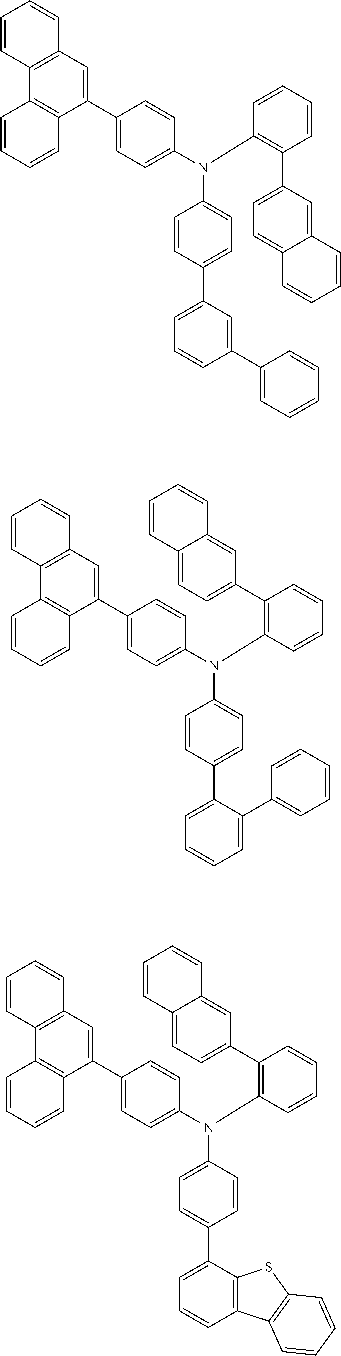 [SMILES: c1ccc(-c2cccc(-c3ccc(N(c4ccc(-c5cc6ccccc6c6ccccc56)cc4)c4ccccc4-c4ccc5ccccc5c4)cc3)c2)cc1 ; c1ccc(-c2ccccc2-c2ccc(N(c3ccc(-c4cc5ccccc5c5ccccc45)cc3)c3ccccc3-c3ccc4ccccc4c3)cc2)cc1 ; c1ccc(N(c2ccc(-c3cc4ccccc4c4ccccc34)cc2)c2ccc(-c3cccc4c3sc3ccccc34)cc2)c(-c2ccc3ccccc3c2)c1]

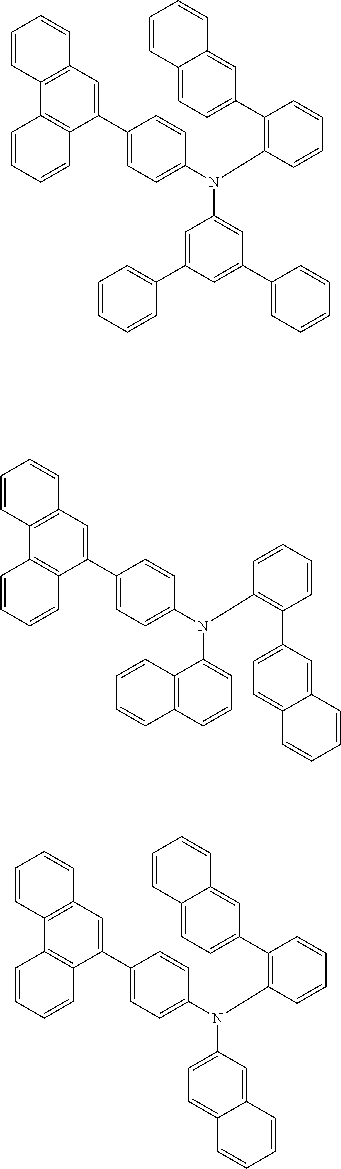 [SMILES: c1ccc(-c2cc(-c3ccccc3)cc(N(c3ccc(-c4cc5ccccc5c5ccccc45)cc3)c3ccccc3-c3ccc4ccccc4c3)c2)cc1 ; c1ccc(N(c2ccc(-c3cc4ccccc4c4ccccc34)cc2)c2cccc3ccccc23)c(-c2ccc3ccccc3c2)c1 ; c1ccc(N(c2ccc(-c3cc4ccccc4c4ccccc34)cc2)c2ccc3ccccc3c2)c(-c2ccc3ccccc3c2)c1]

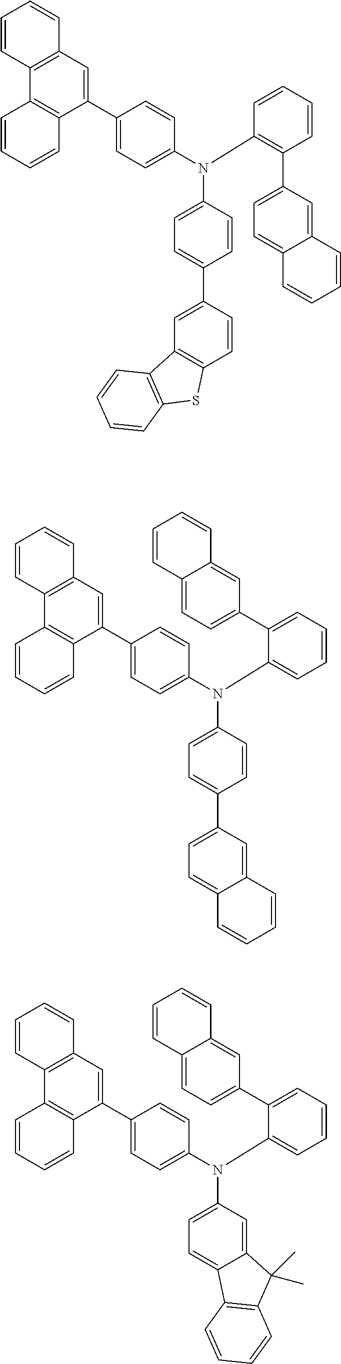 [SMILES: c1ccc(N(c2ccc(-c3ccc4sc5ccccc5c4c3)cc2)c2ccc(-c3cc4ccccc4c4ccccc34)cc2)c(-c2ccc3ccccc3c2)c1 ; c1ccc(N(c2ccc(-c3ccc4ccccc4c3)cc2)c2ccc(-c3cc4ccccc4c4ccccc34)cc2)c(-c2ccc3ccccc3c2)c1 ; CC1(C)c2ccccc2-c2ccc(N(c3ccc(-c4cc5ccccc5c5ccccc45)cc3)c3ccccc3-c3ccc4ccccc4c3)cc21]

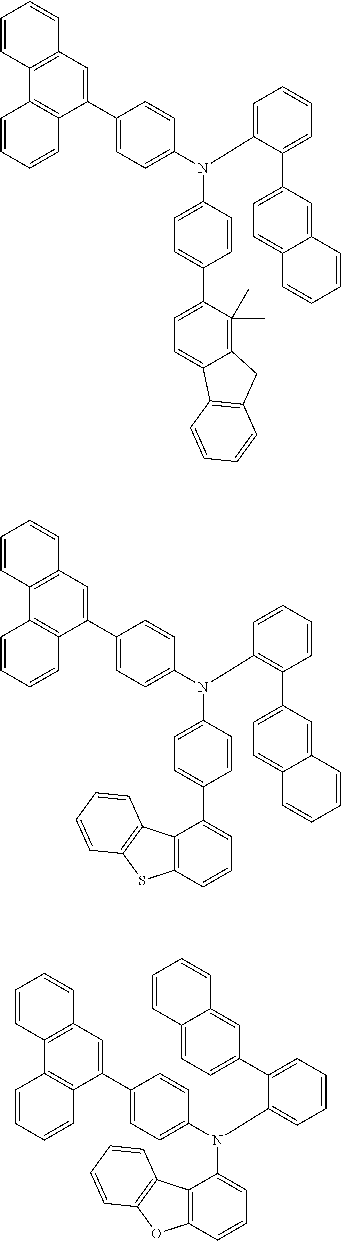

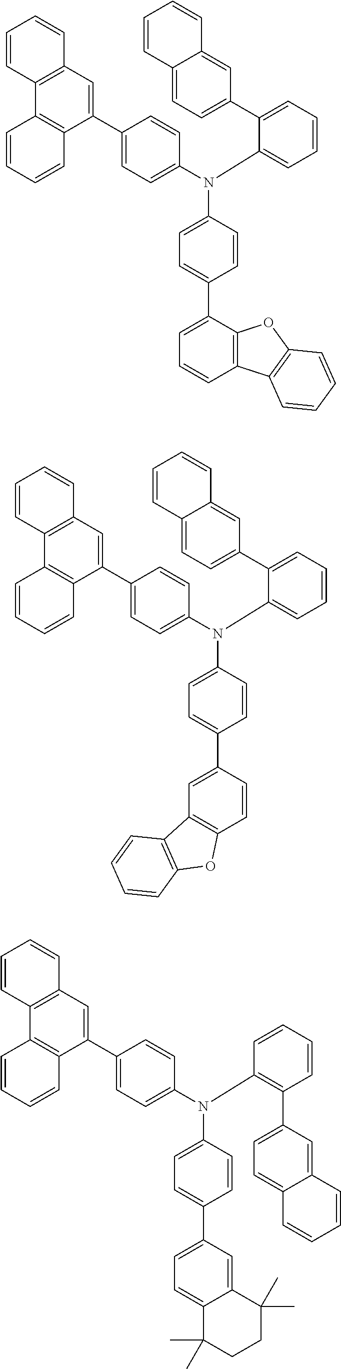 [SMILES: c1ccc(N(c2ccc(-c3cc4ccccc4c4ccccc34)cc2)c2ccc(-c3cccc4c3oc3ccccc34)cc2)c(-c2ccc3ccccc3c2)c1 ; c1ccc(N(c2ccc(-c3ccc4oc5ccccc5c4c3)cc2)c2ccc(-c3cc4ccccc4c4ccccc34)cc2)c(-c2ccc3ccccc3c2)c1 ; CC1(C)CCC(C)(C)c2cc(-c3ccc(N(c4ccc(-c5cc6ccccc6c6ccccc56)cc4)c4ccccc4-c4ccc5ccccc5c4)cc3)ccc21]

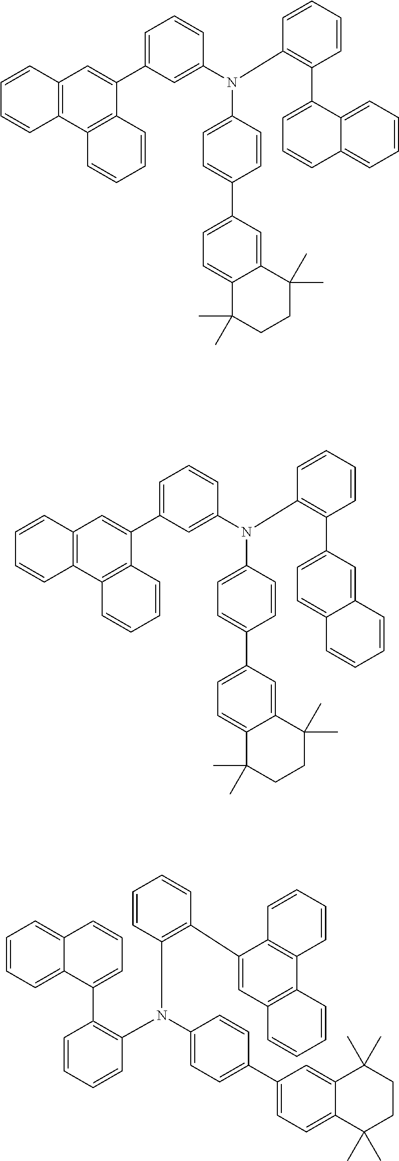 [SMILES: CC1(C)CCC(C)(C)c2cc(-c3ccc(N(c4cccc(-c5cc6ccccc6c6ccccc56)c4)c4ccccc4-c4cccc5ccccc45)cc3)ccc21 ; CC1(C)CCC(C)(C)c2cc(-c3ccc(N(c4cccc(-c5cc6ccccc6c6ccccc56)c4)c4ccccc4-c4ccc5ccccc5c4)cc3)ccc21 ; CC1(C)CCC(C)(C)c2cc(-c3ccc(N(c4ccccc4-c4cccc5ccccc45)c4ccccc4-c4cc5ccccc5c5ccccc45)cc3)ccc21]

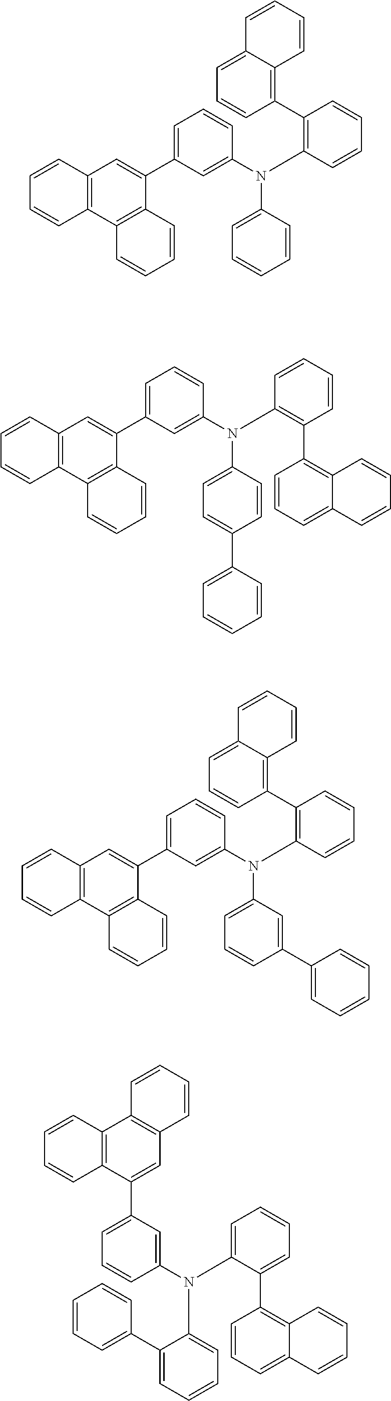 [SMILES: c1ccc(N(c2cccc(-c3cc4ccccc4c4ccccc34)c2)c2ccccc2-c2cccc3ccccc23)cc1 ; c1ccc(-c2ccc(N(c3cccc(-c4cc5ccccc5c5ccccc45)c3)c3ccccc3-c3cccc4ccccc34)cc2)cc1 ; c1ccc(-c2cccc(N(c3cccc(-c4cc5ccccc5c5ccccc45)c3)c3ccccc3-c3cccc4ccccc34)c2)cc1 ; c1ccc(-c2ccccc2N(c2cccc(-c3cc4ccccc4c4ccccc34)c2)c2ccccc2-c2cccc3ccccc23)cc1]

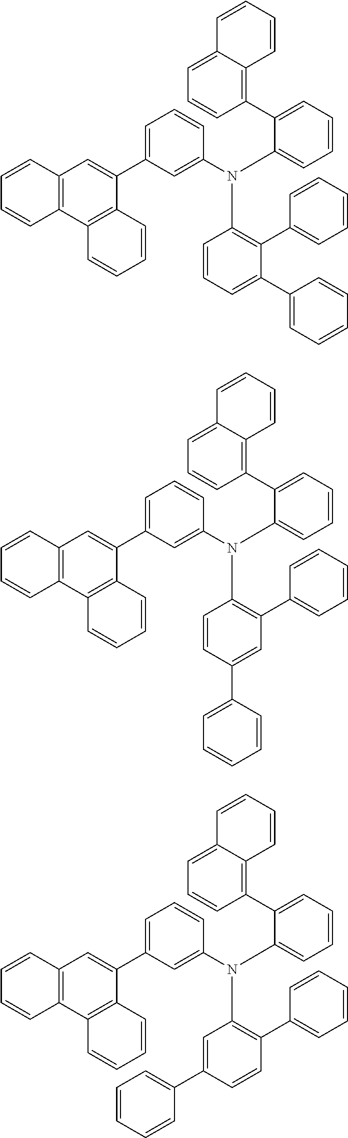

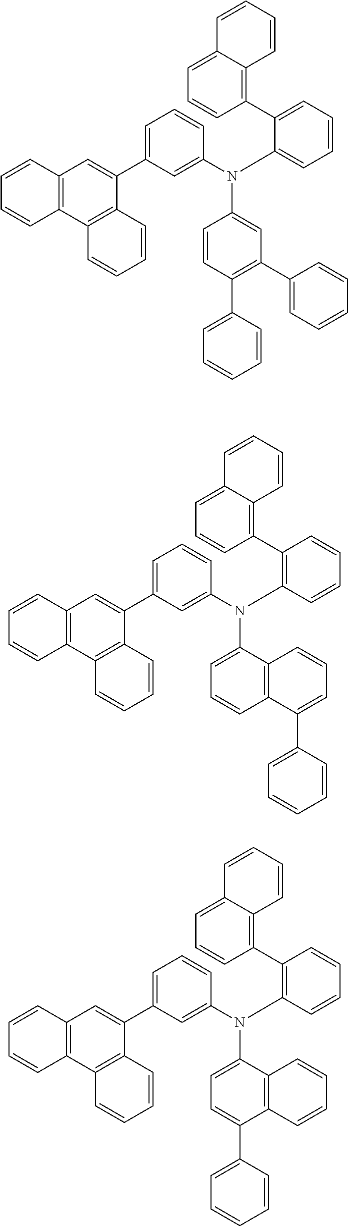 [SMILES: c1ccc(-c2ccc(N(c3cccc(-c4cc5ccccc5c5ccccc45)c3)c3ccccc3-c3cccc4ccccc34)cc2-c2ccccc2)cc1 ; c1ccc(-c2cccc3c(N(c4cccc(-c5cc6ccccc6c6ccccc56)c4)c4ccccc4-c4cccc5ccccc45)cccc23)cc1 ; c1ccc(-c2ccc(N(c3cccc(-c4cc5ccccc5c5ccccc45)c3)c3ccccc3-c3cccc4ccccc34)c3ccccc23)cc1]

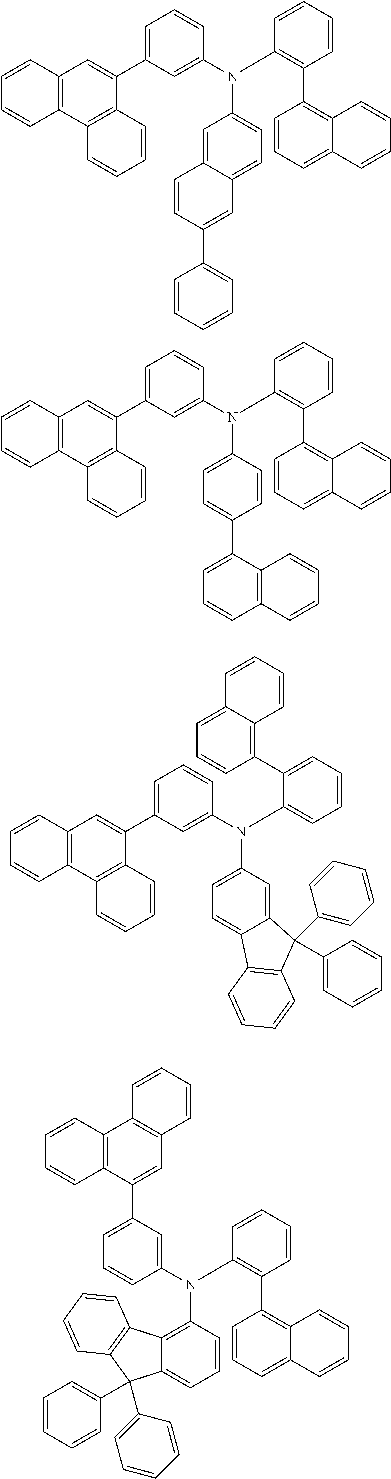

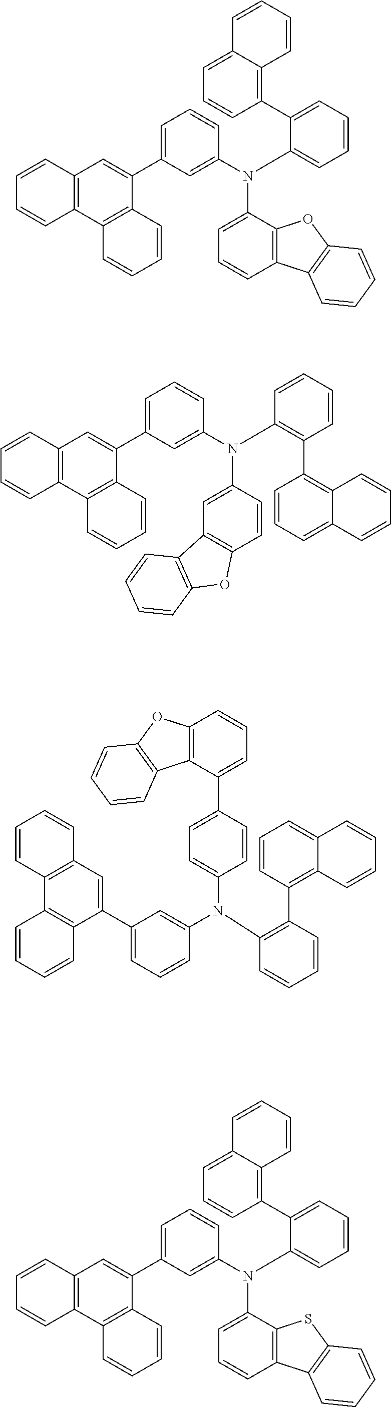 [SMILES: c1cc(-c2cc3ccccc3c3ccccc23)cc(N(c2ccccc2-c2cccc3ccccc23)c2cccc3c2oc2ccccc23)c1 ; c1cc(-c2cc3ccccc3c3ccccc23)cc(N(c2ccc3oc4ccccc4c3c2)c2ccccc2-c2cccc3ccccc23)c1 ; c1cc(-c2cc3ccccc3c3ccccc23)cc(N(c2ccc(-c3cccc4oc5ccccc5c34)cc2)c2ccccc2-c2cccc3ccccc23)c1 ; c1cc(-c2cc3ccccc3c3ccccc23)cc(N(c2ccccc2-c2cccc3ccccc23)c2cccc3c2sc2ccccc23)c1]

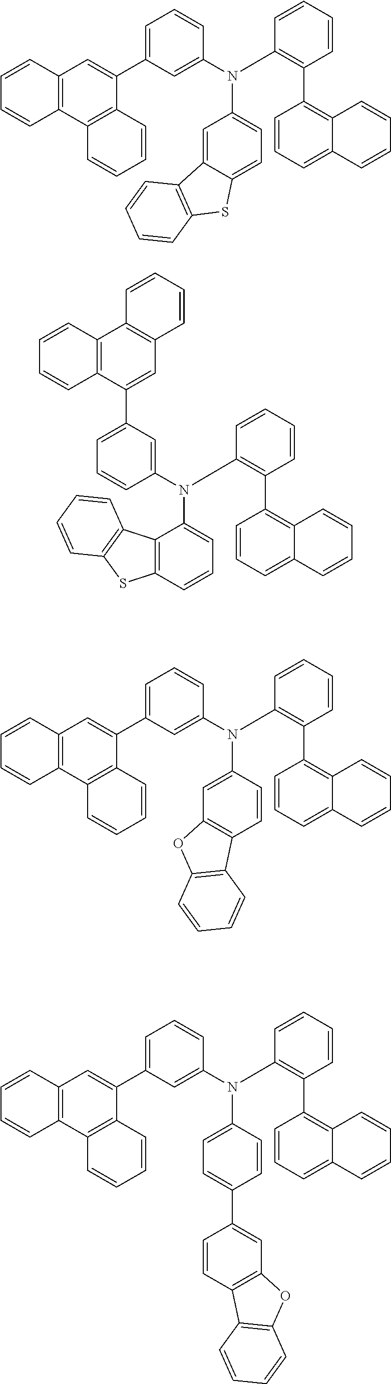

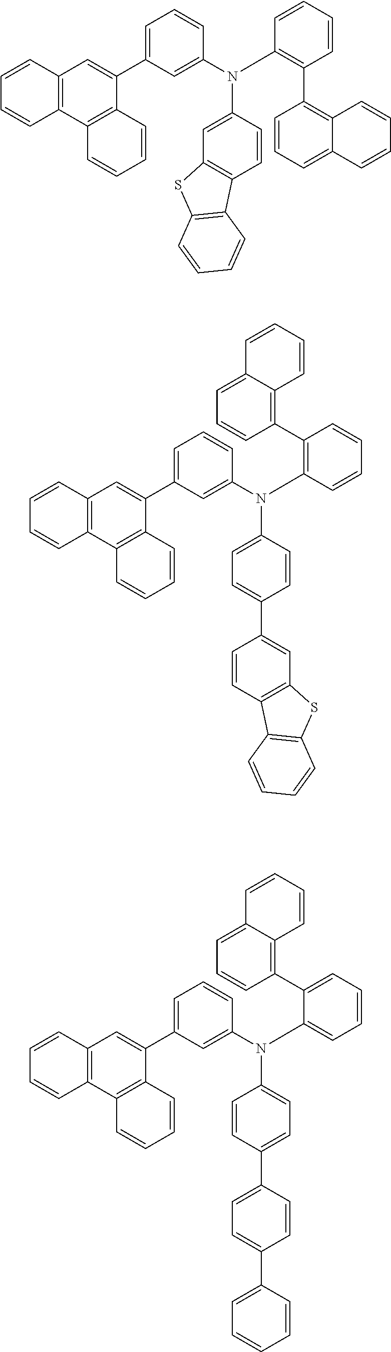 [SMILES: c1cc(-c2cc3ccccc3c3ccccc23)cc(N(c2ccc3c(c2)sc2ccccc23)c2ccccc2-c2cccc3ccccc23)c1 ; c1cc(-c2cc3ccccc3c3ccccc23)cc(N(c2ccc(-c3ccc4c(c3)sc3ccccc34)cc2)c2ccccc2-c2cccc3ccccc23)c1 ; c1ccc(-c2ccc(-c3ccc(N(c4cccc(-c5cc6ccccc6c6ccccc56)c4)c4ccccc4-c4cccc5ccccc45)cc3)cc2)cc1]

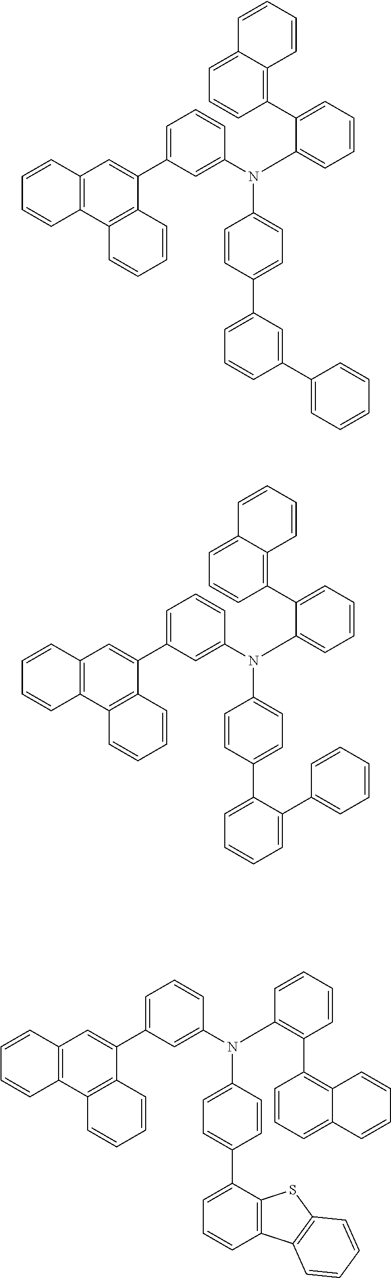 [SMILES: c1ccc(-c2cccc(-c3ccc(N(c4cccc(-c5cc6ccccc6c6ccccc56)c4)c4ccccc4-c4cccc5ccccc45)cc3)c2)cc1 ; c1ccc(-c2ccccc2-c2ccc(N(c3cccc(-c4cc5ccccc5c5ccccc45)c3)c3ccccc3-c3cccc4ccccc34)cc2)cc1 ; c1cc(-c2cc3ccccc3c3ccccc23)cc(N(c2ccc(-c3cccc4c3sc3ccccc34)cc2)c2ccccc2-c2cccc3ccccc23)c1]

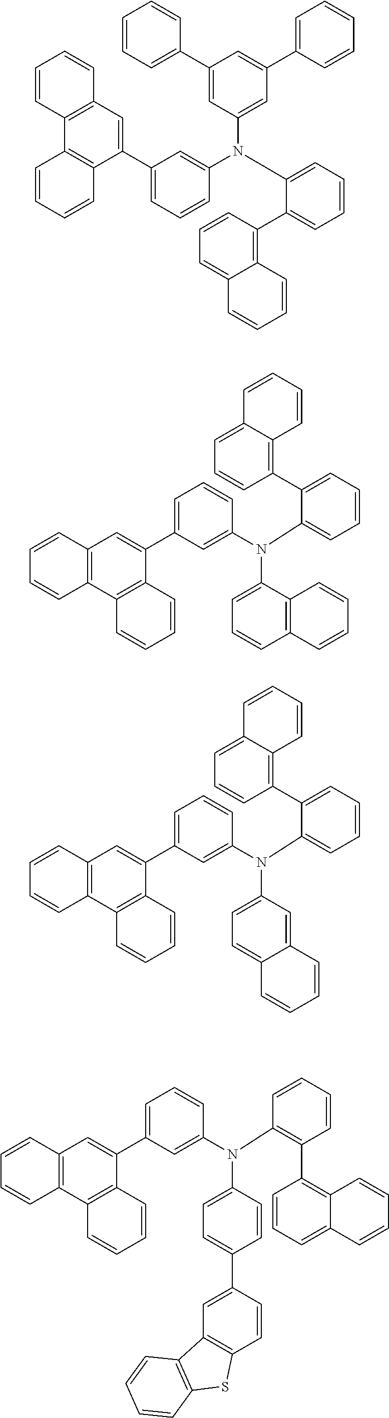

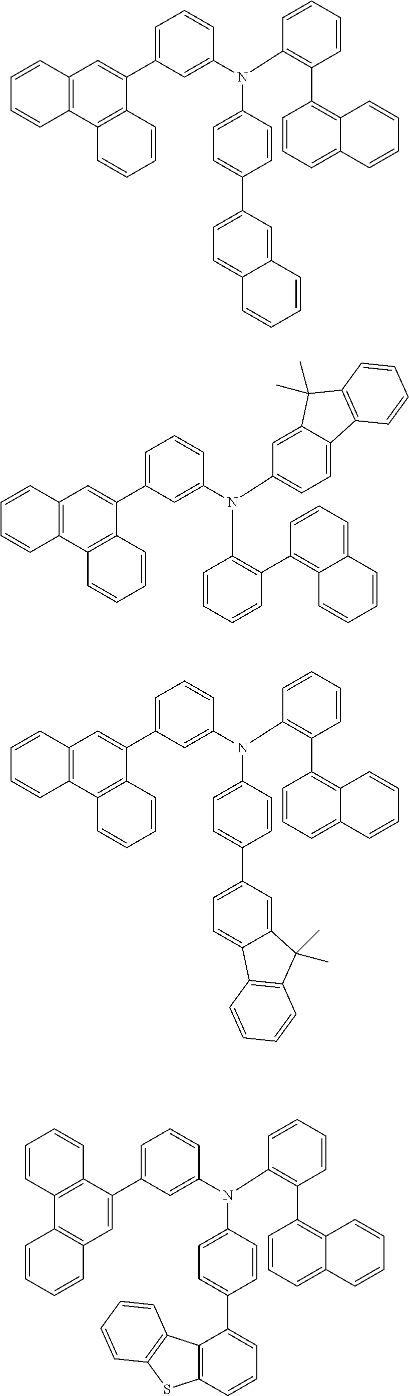

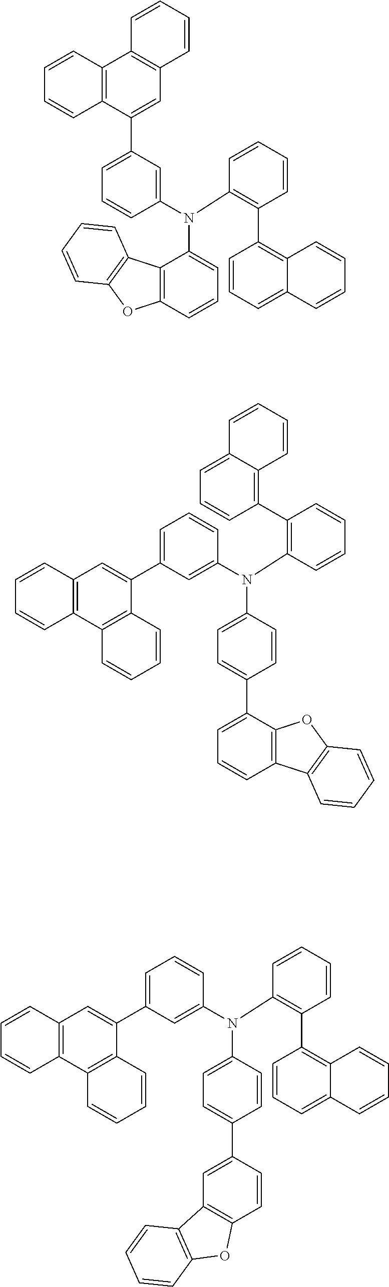 [SMILES: c1cc(-c2cc3ccccc3c3ccccc23)cc(N(c2ccccc2-c2cccc3ccccc23)c2cccc3oc4ccccc4c23)c1 ; c1cc(-c2cc3ccccc3c3ccccc23)cc(N(c2ccc(-c3cccc4c3oc3ccccc34)cc2)c2ccccc2-c2cccc3ccccc23)c1 ; c1cc(-c2cc3ccccc3c3ccccc23)cc(N(c2ccc(-c3ccc4oc5ccccc5c4c3)cc2)c2ccccc2-c2cccc3ccccc23)c1]

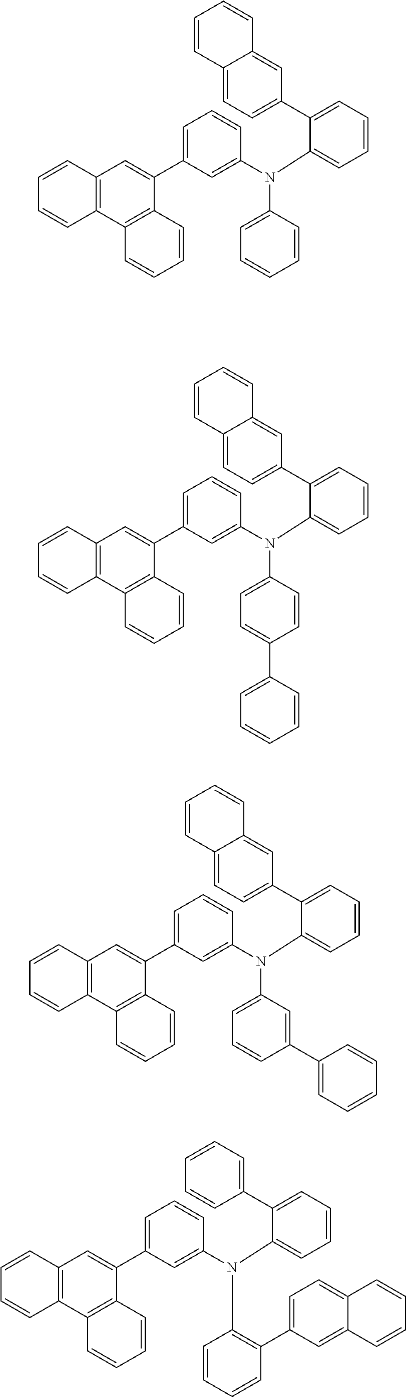 [SMILES: c1ccc(N(c2cccc(-c3cc4ccccc4c4ccccc34)c2)c2ccccc2-c2ccc3ccccc3c2)cc1 ; c1ccc(-c2ccc(N(c3cccc(-c4cc5ccccc5c5ccccc45)c3)c3ccccc3-c3ccc4ccccc4c3)cc2)cc1 ; c1ccc(-c2cccc(N(c3cccc(-c4cc5ccccc5c5ccccc45)c3)c3ccccc3-c3ccc4ccccc4c3)c2)cc1 ; c1ccc(-c2ccccc2N(c2cccc(-c3cc4ccccc4c4ccccc34)c2)c2ccccc2-c2ccc3ccccc3c2)cc1]

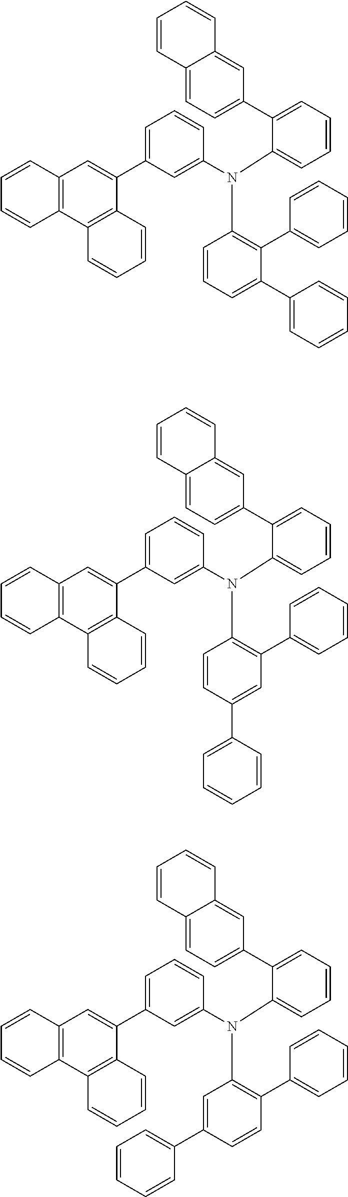 [SMILES: c1ccc(-c2cccc(N(c3cccc(-c4cc5ccccc5c5ccccc45)c3)c3ccccc3-c3ccc4ccccc4c3)c2-c2ccccc2)cc1 ; c1ccc(-c2ccc(N(c3cccc(-c4cc5ccccc5c5ccccc45)c3)c3ccccc3-c3ccc4ccccc4c3)c(-c3ccccc3)c2)cc1 ; c1ccc(-c2ccc(-c3ccccc3)c(N(c3cccc(-c4cc5ccccc5c5ccccc45)c3)c3ccccc3-c3ccc4ccccc4c3)c2)cc1]

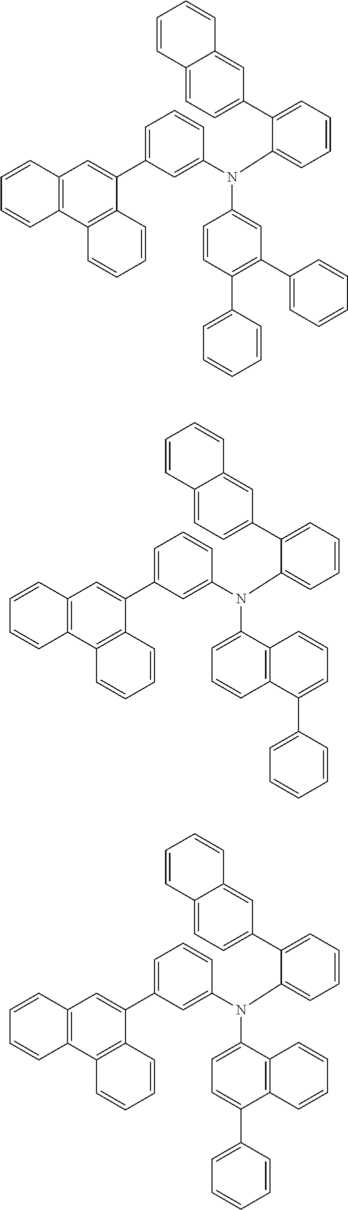 [SMILES: c1ccc(-c2ccc(N(c3cccc(-c4cc5ccccc5c5ccccc45)c3)c3ccccc3-c3ccc4ccccc4c3)cc2-c2ccccc2)cc1 ; c1ccc(-c2cccc3c(N(c4cccc(-c5cc6ccccc6c6ccccc56)c4)c4ccccc4-c4ccc5ccccc5c4)cccc23)cc1 ; c1ccc(-c2ccc(N(c3cccc(-c4cc5ccccc5c5ccccc45)c3)c3ccccc3-c3ccc4ccccc4c3)c3ccccc23)cc1]

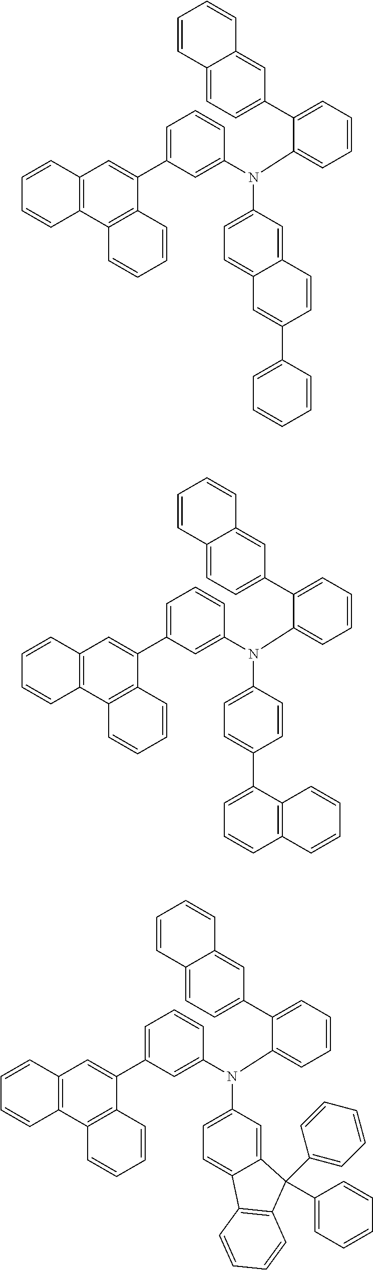 [SMILES: c1ccc(-c2ccc3cc(N(c4cccc(-c5cc6ccccc6c6ccccc56)c4)c4ccccc4-c4ccc5ccccc5c4)ccc3c2)cc1 ; c1cc(-c2cc3ccccc3c3ccccc23)cc(N(c2ccc(-c3cccc4ccccc34)cc2)c2ccccc2-c2ccc3ccccc3c2)c1 ; c1ccc(C2(c3ccccc3)c3ccccc3-c3ccc(N(c4cccc(-c5cc6ccccc6c6ccccc56)c4)c4ccccc4-c4ccc5ccccc5c4)cc32)cc1]

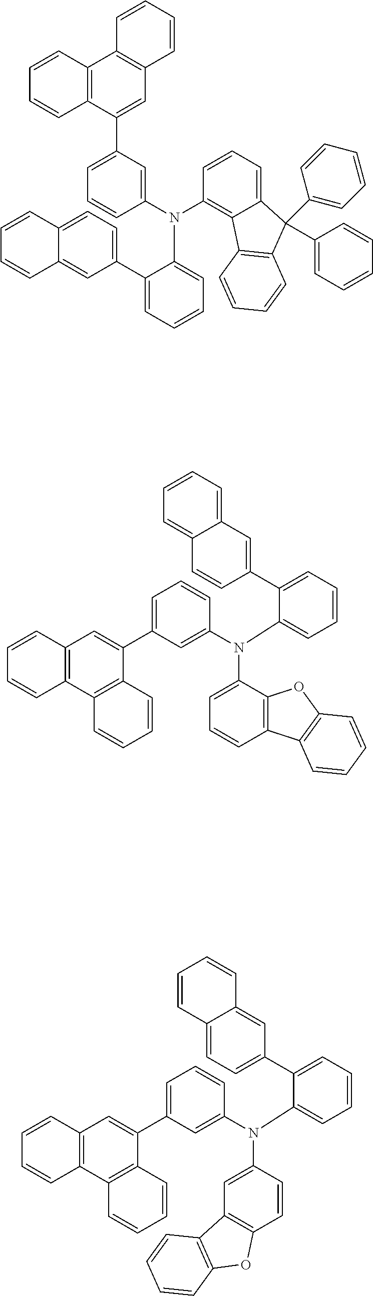 [SMILES: c1ccc(C2(c3ccccc3)c3ccccc3-c3c(N(c4cccc(-c5cc6ccccc6c6ccccc56)c4)c4ccccc4-c4ccc5ccccc5c4)cccc32)cc1 ; c1cc(-c2cc3ccccc3c3ccccc23)cc(N(c2ccccc2-c2ccc3ccccc3c2)c2cccc3c2oc2ccccc23)c1 ; c1cc(-c2cc3ccccc3c3ccccc23)cc(N(c2ccc3oc4ccccc4c3c2)c2ccccc2-c2ccc3ccccc3c2)c1]

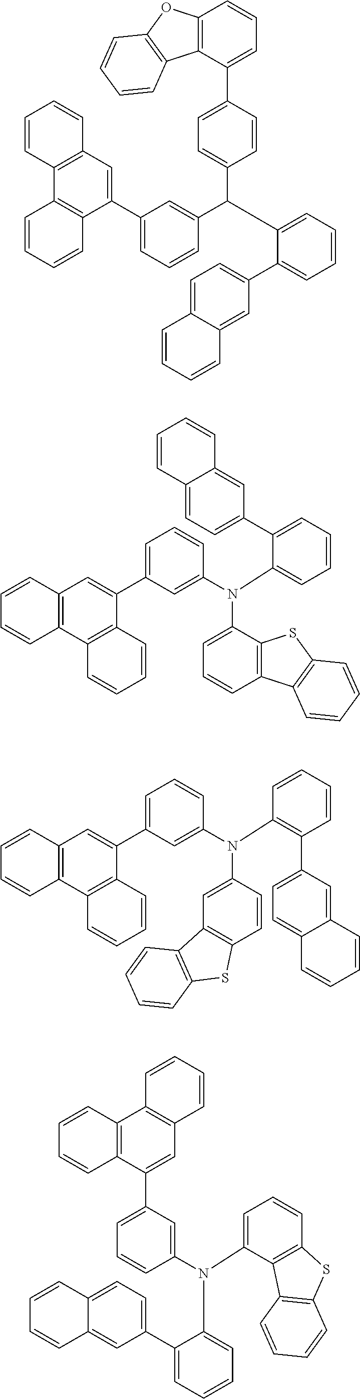

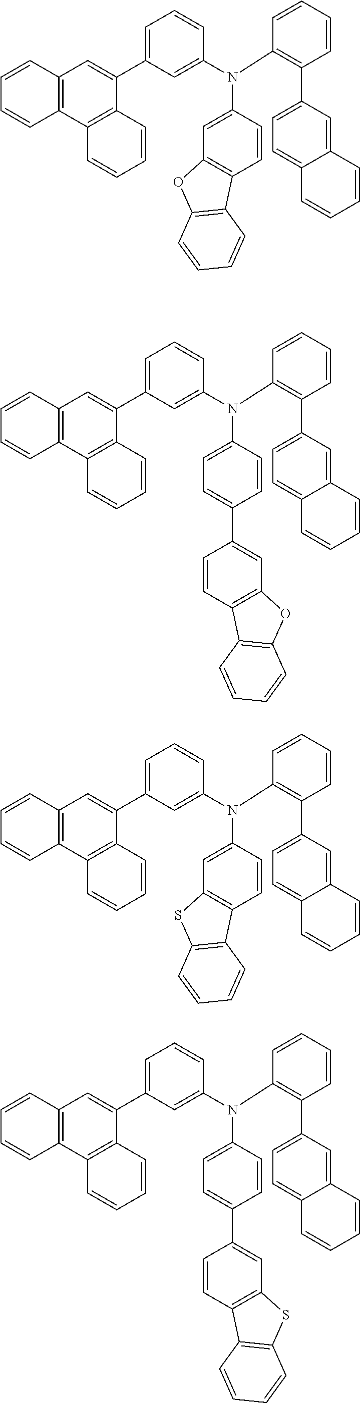

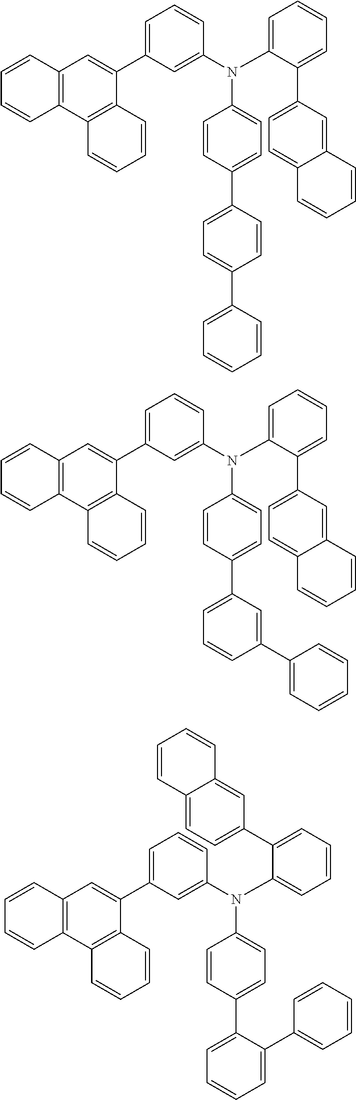 [SMILES: c1ccc(-c2ccc(-c3ccc(N(c4cccc(-c5cc6ccccc6c6ccccc56)c4)c4ccccc4-c4ccc5ccccc5c4)cc3)cc2)cc1 ; c1ccc(-c2cccc(-c3ccc(N(c4cccc(-c5cc6ccccc6c6ccccc56)c4)c4ccccc4-c4ccc5ccccc5c4)cc3)c2)cc1 ; c1ccc(-c2ccccc2-c2ccc(N(c3cccc(-c4cc5ccccc5c5ccccc45)c3)c3ccccc3-c3ccc4ccccc4c3)cc2)cc1]

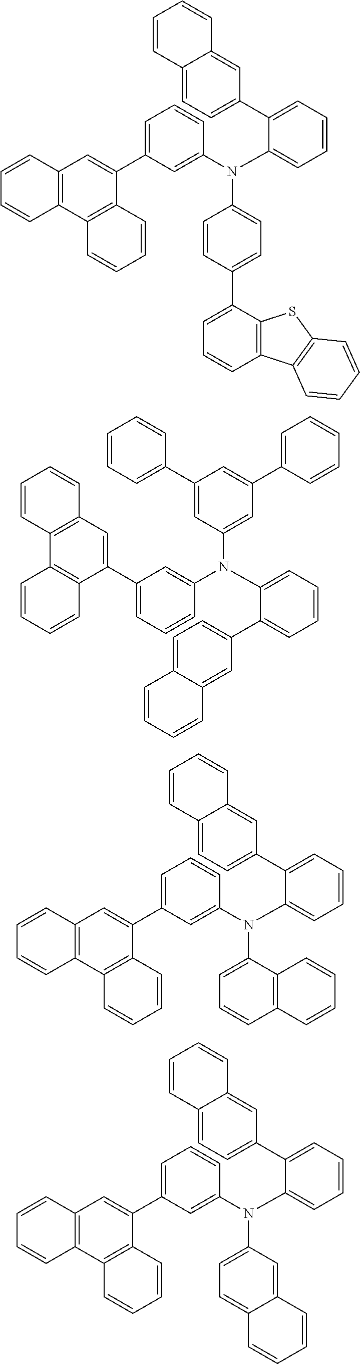

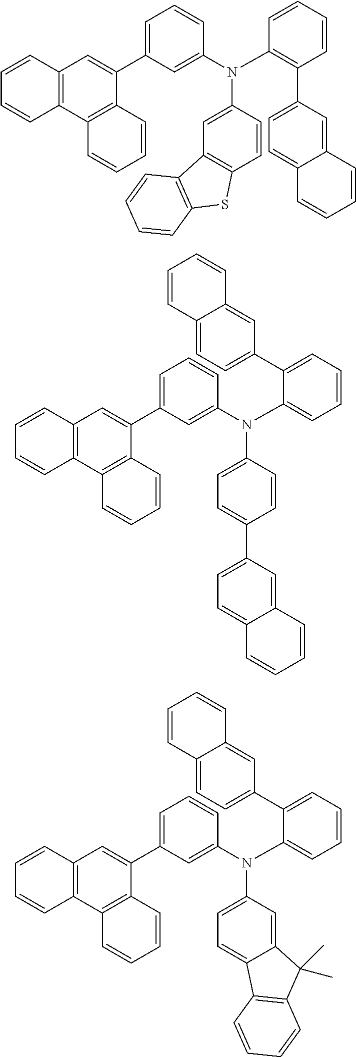

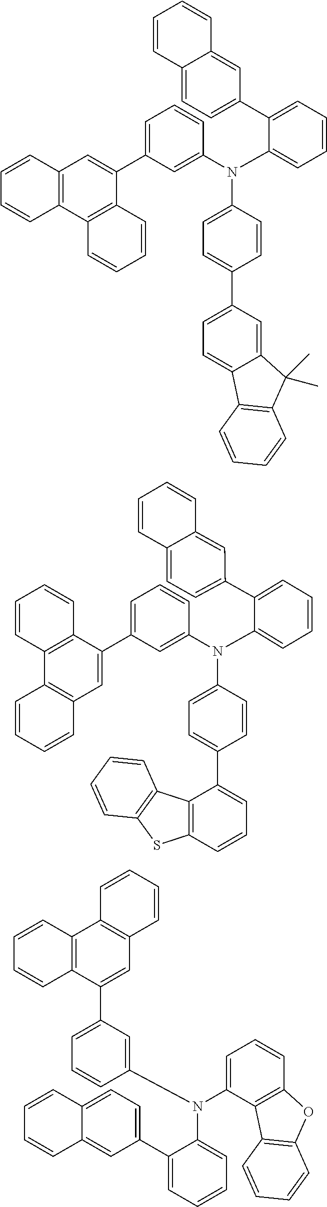

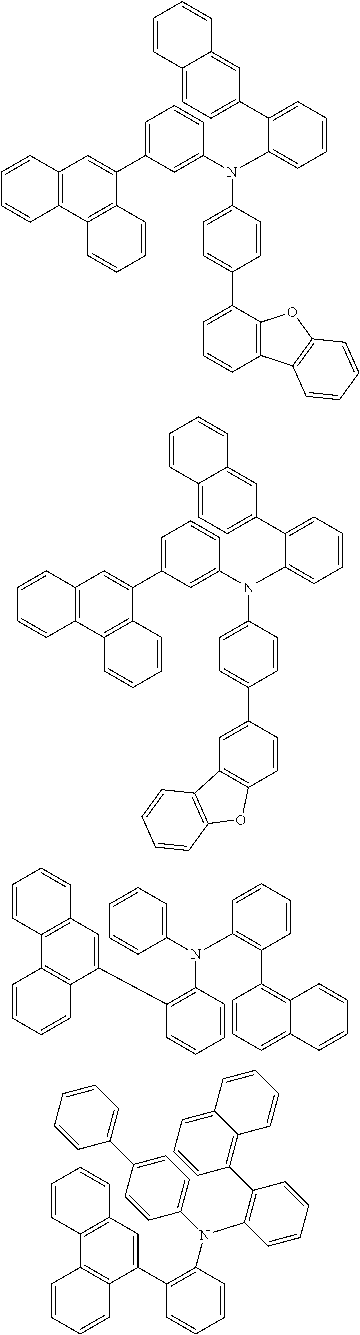

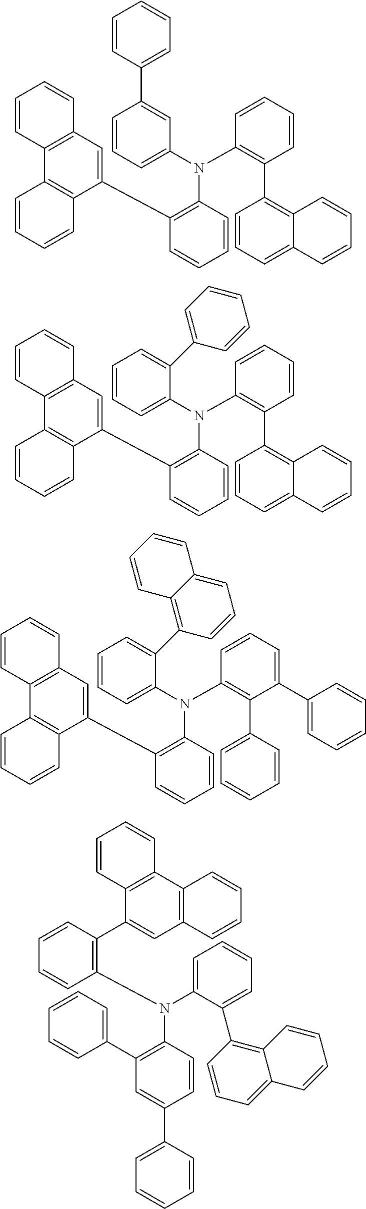

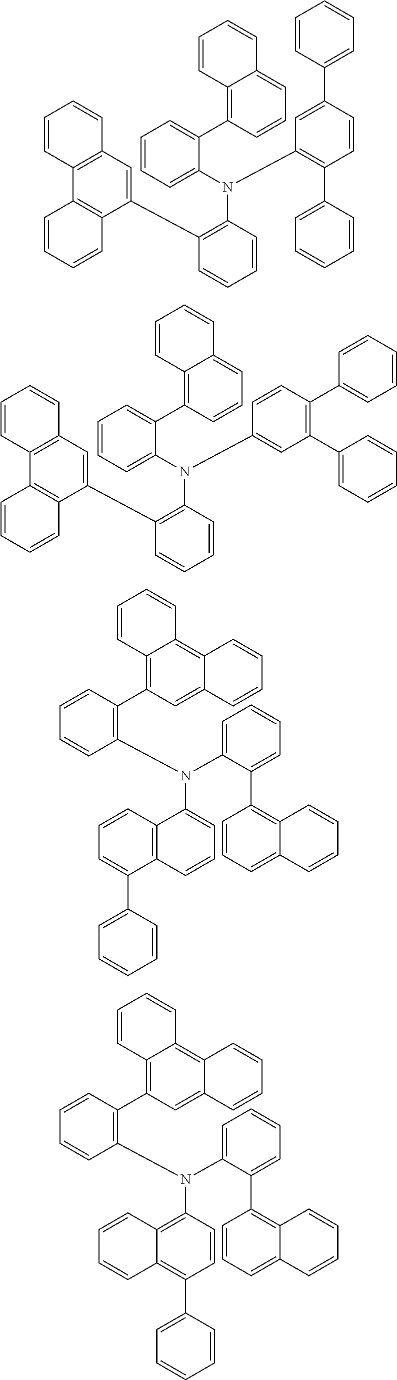

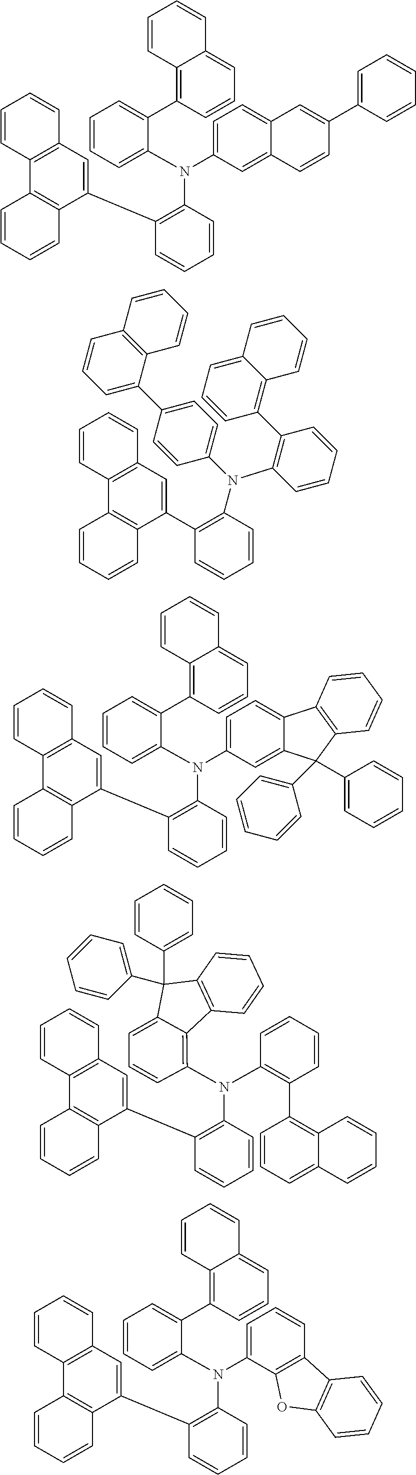

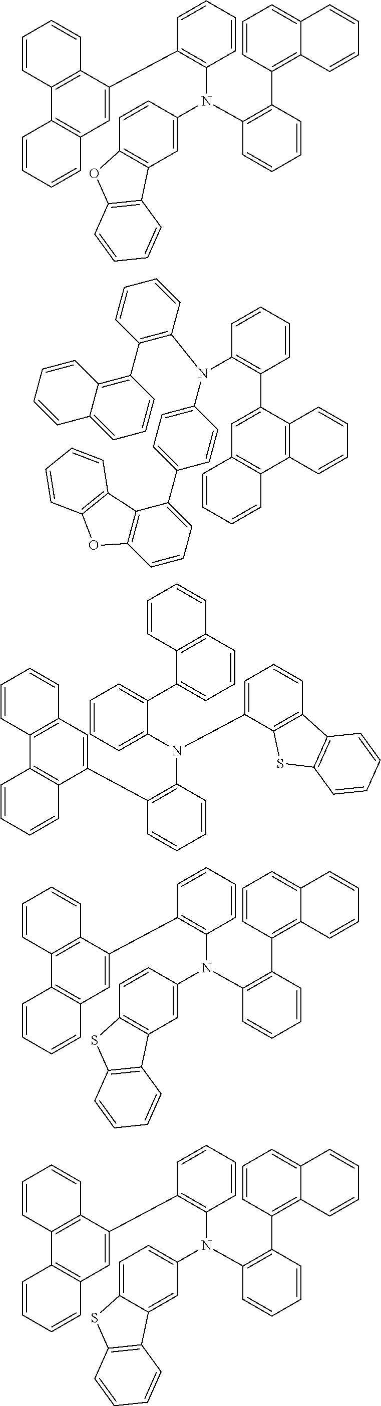

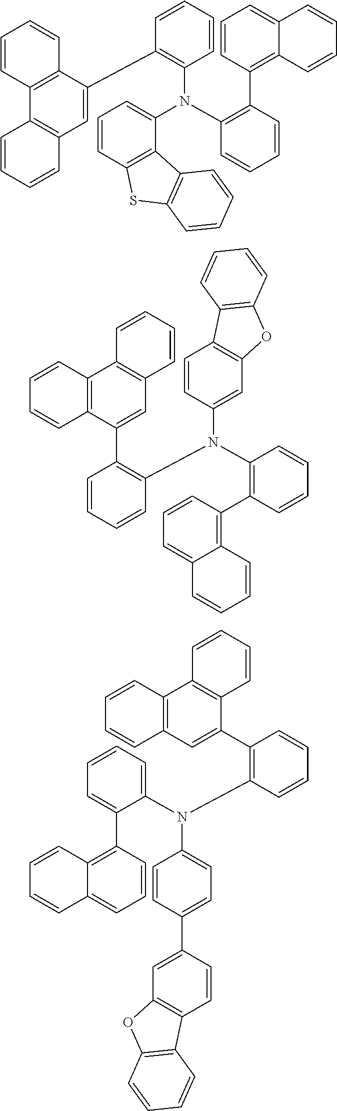

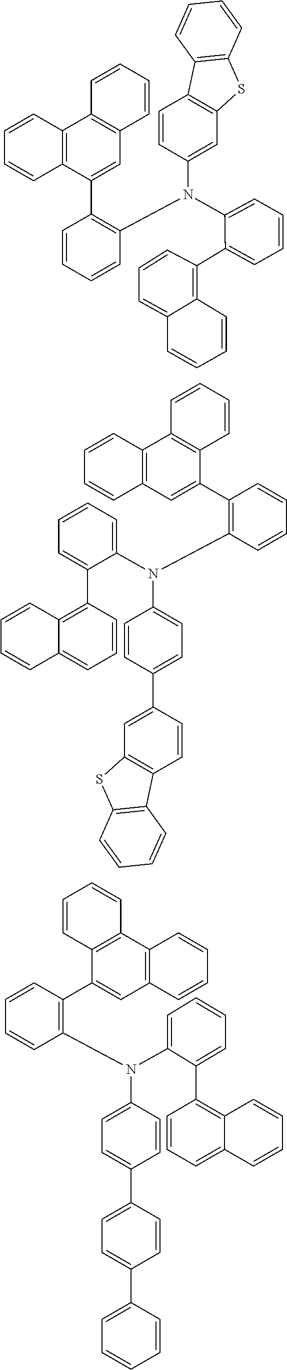

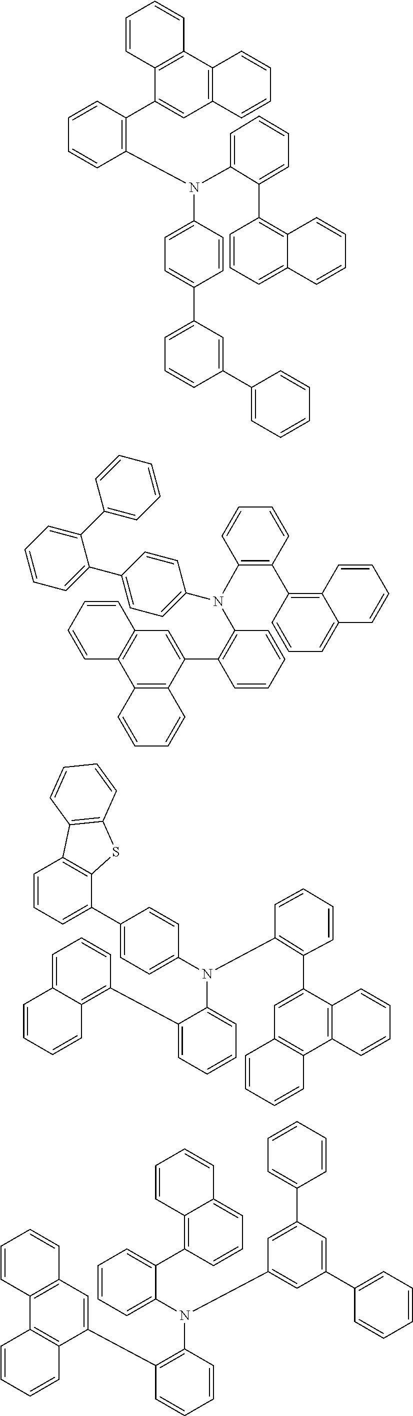

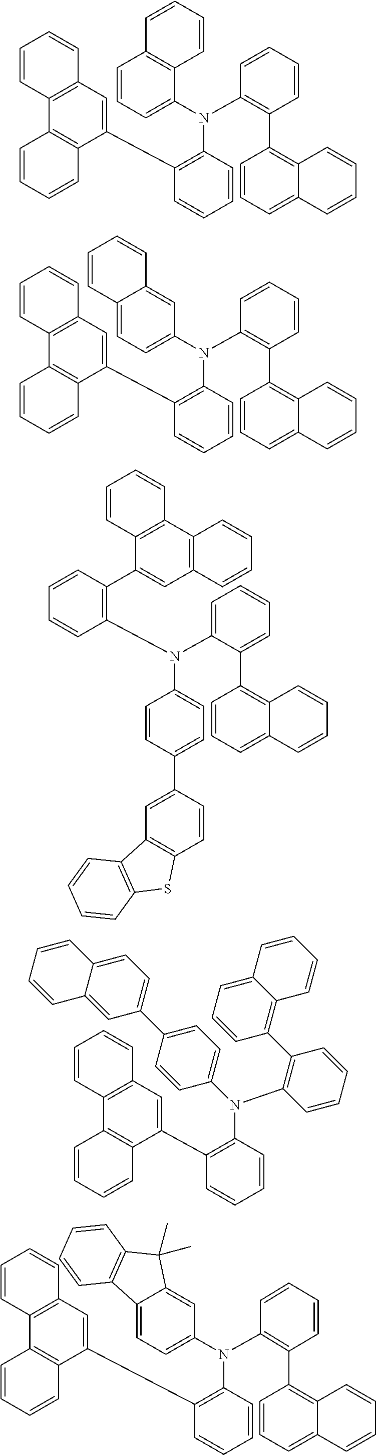

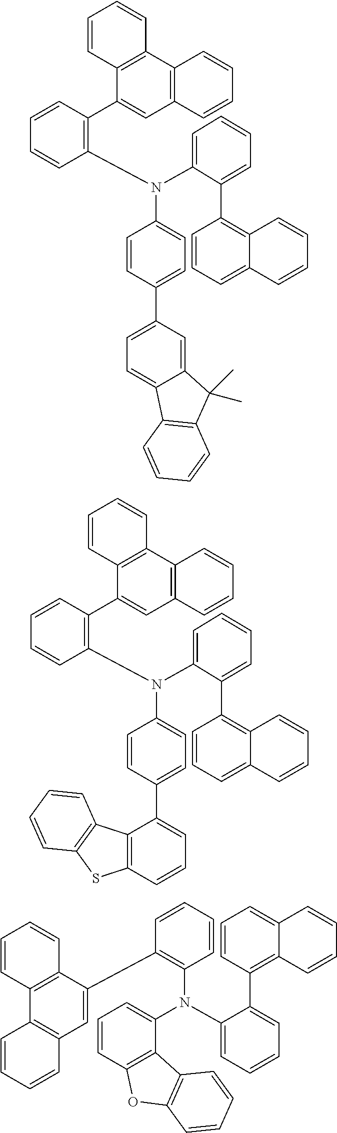

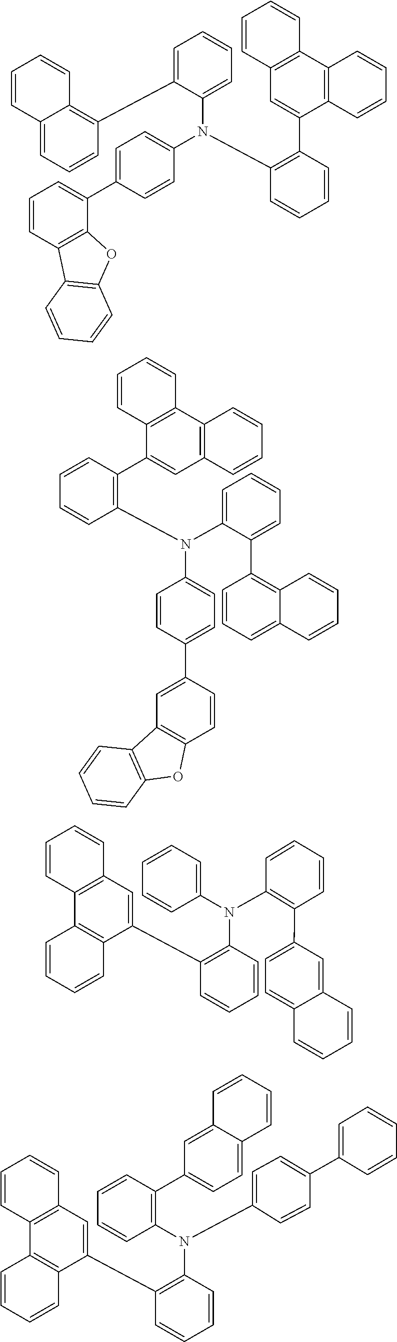

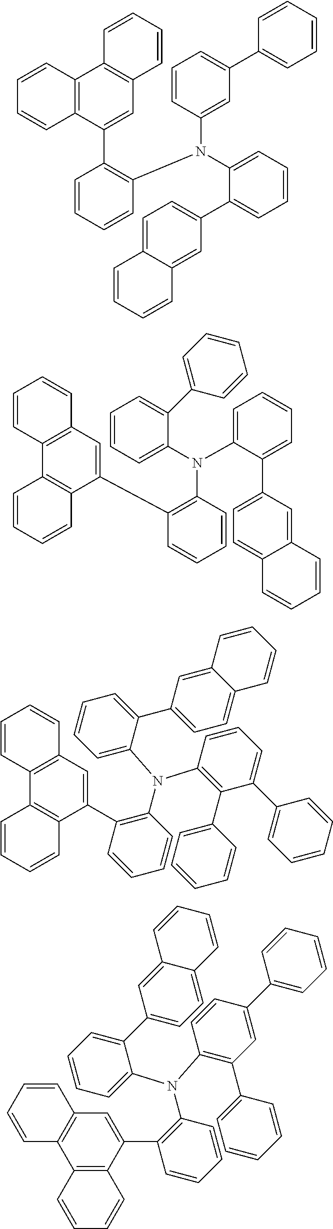

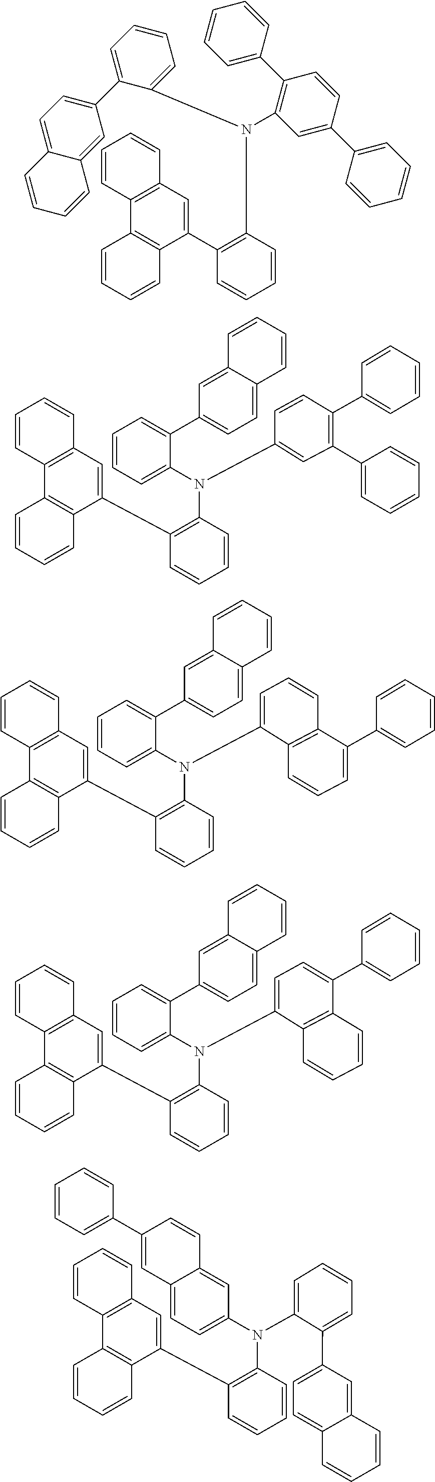

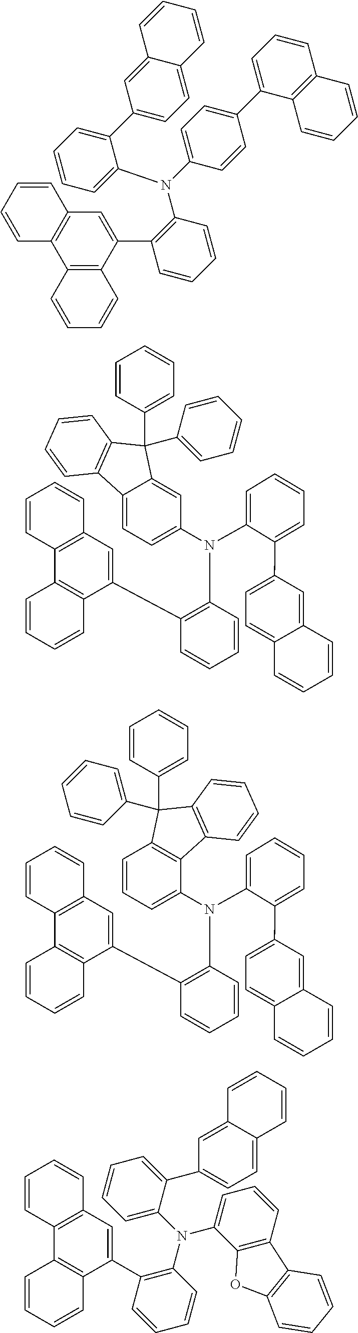

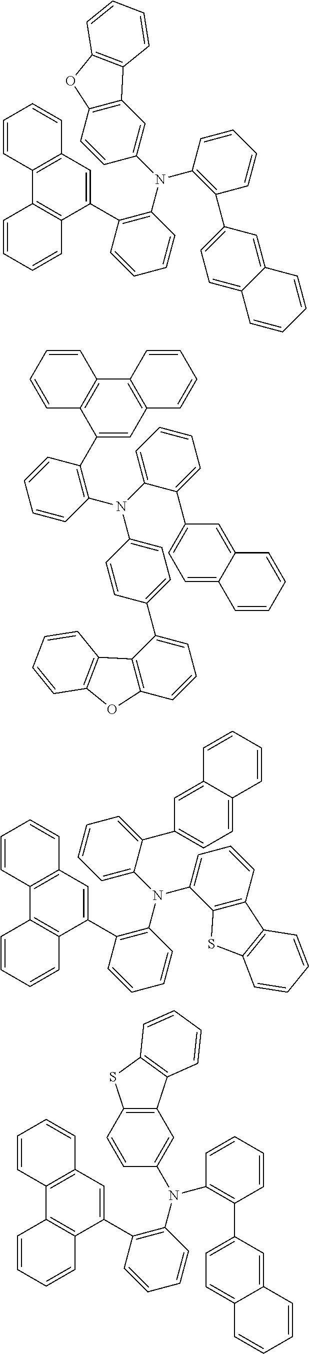

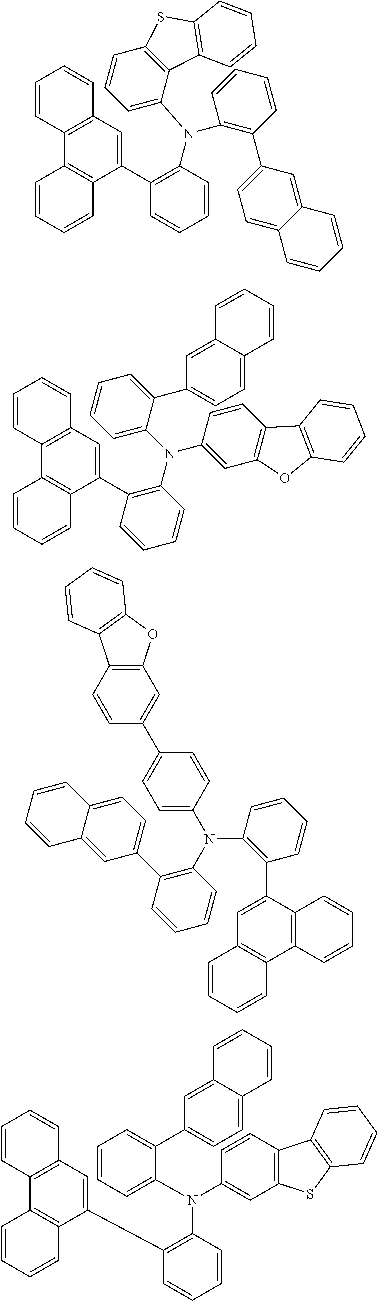

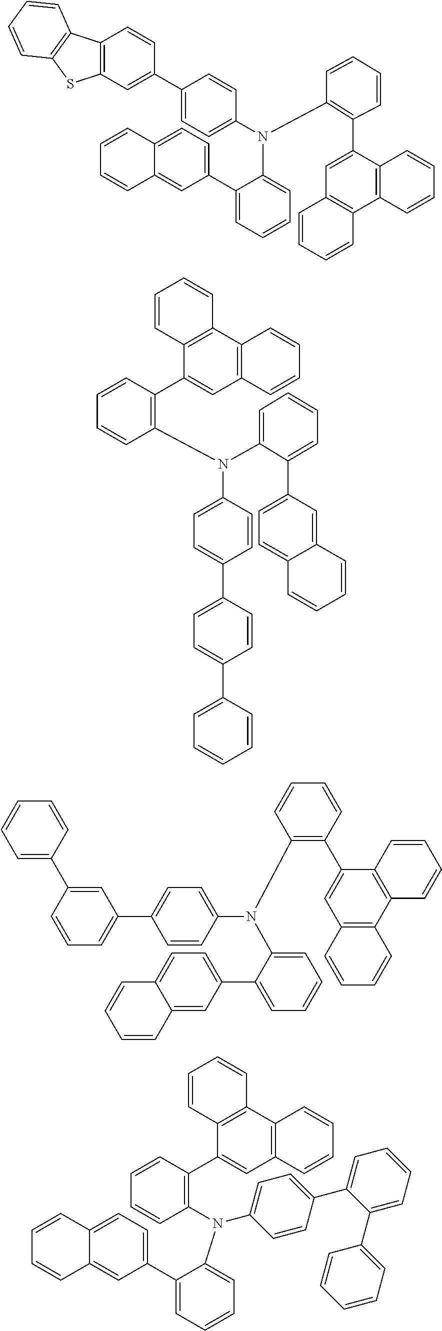 [SMILES: c1ccc(N(c2ccc(-c3ccc4c(c3)sc3ccccc34)cc2)c2ccccc2-c2cc3ccccc3c3ccccc23)c(-c2ccc3ccccc3c2)c1 ; c1ccc(-c2ccc(-c3ccc(N(c4ccccc4-c4ccc5ccccc5c4)c4ccccc4-c4cc5ccccc5c5ccccc45)cc3)cc2)cc1 ; c1ccc(-c2cccc(-c3ccc(N(c4ccccc4-c4ccc5ccccc5c4)c4ccccc4-c4cc5ccccc5c5ccccc45)cc3)c2)cc1 ; c1ccc(-c2ccccc2-c2ccc(N(c3ccccc3-c3ccc4ccccc4c3)c3ccccc3-c3cc4ccccc4c4ccccc34)cc2)cc1]

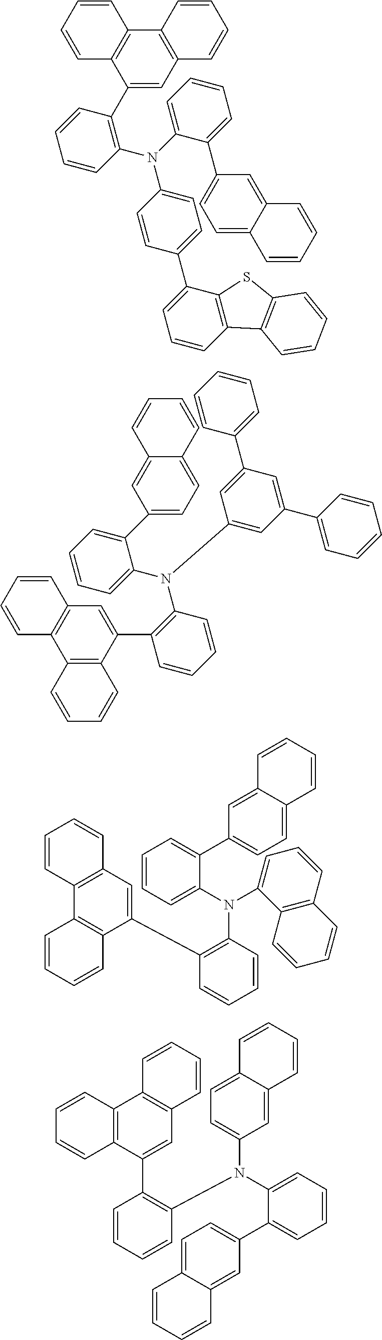

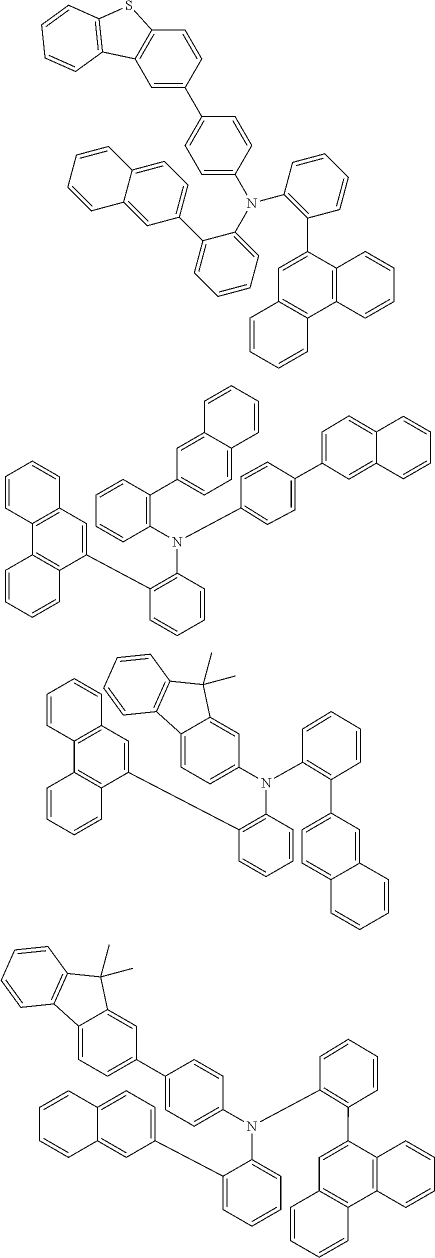

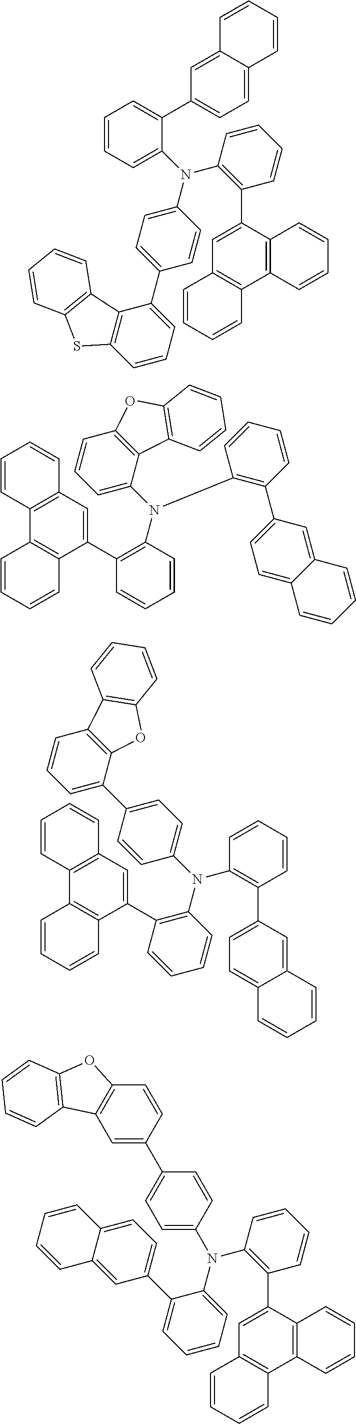

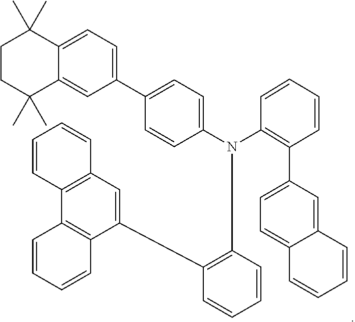 [SMILES: CC1(C)CCC(C)(C)c2cc(-c3ccc(N(c4ccccc4-c4ccc5ccccc5c4)c4ccccc4-c4cc5ccccc5c5ccccc45)cc3)ccc21 ; "."]

10. The organic light emitting device of claim 1, wherein the first organic material layer is provided to be brought into direct contact with the light emitting layer.

11. The organic light emitting device of claim 1, wherein the light emitting layer has a maximum light emission peak of 400 nm to 500 nm.

12. The organic light emitting device of claim 1, wherein the light emitting layer comprises a host and a dopant, and the dopant comprises the compound of Chemical Formula 1.

13. The organic light emitting device of claim 12, wherein the host comprises a compound of the following Chemical Formula H: 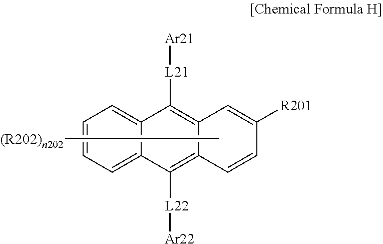
wherein in Chemical Formula H;
L21 and L22 are the same as or different from each other, and are each independently a direct bond, a substituted or unsubstituted arylene group, or a substituted or unsubstituted heteroarylene group,
Ar21 and Ar2 are the same as or different from each other, and are each independently a substituted or unsubstituted aryl group or a substituted or unsubstituted heterocyclic group;
R201 and R202 are the same as or different from each other, and are each independently hydrogen, deuterium, a halogen group, a substituted or unsubstituted alkyl group, a substituted or unsubstituted cycloalkyl group, a substituted or unsubstituted aryl group, or a substituted or unsubstituted heterocyclic group; and
n202 is an integer from 0 to 7, and when n202 is 2 or higher, the R202s are the same as or different from each other.

14. The organic light emitting device of claim 12, wherein the host comprises a compound selected from among one of the following compounds:

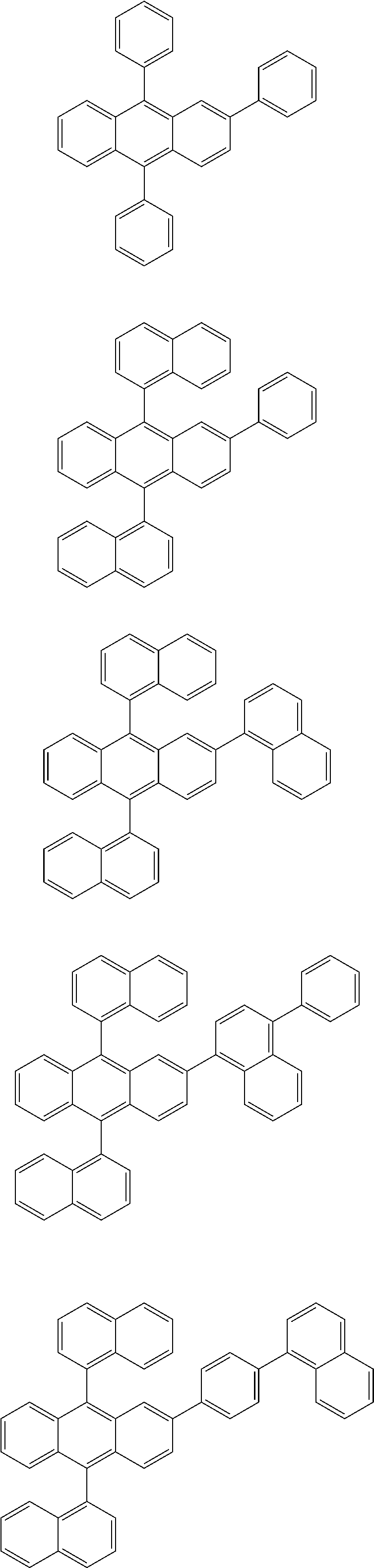

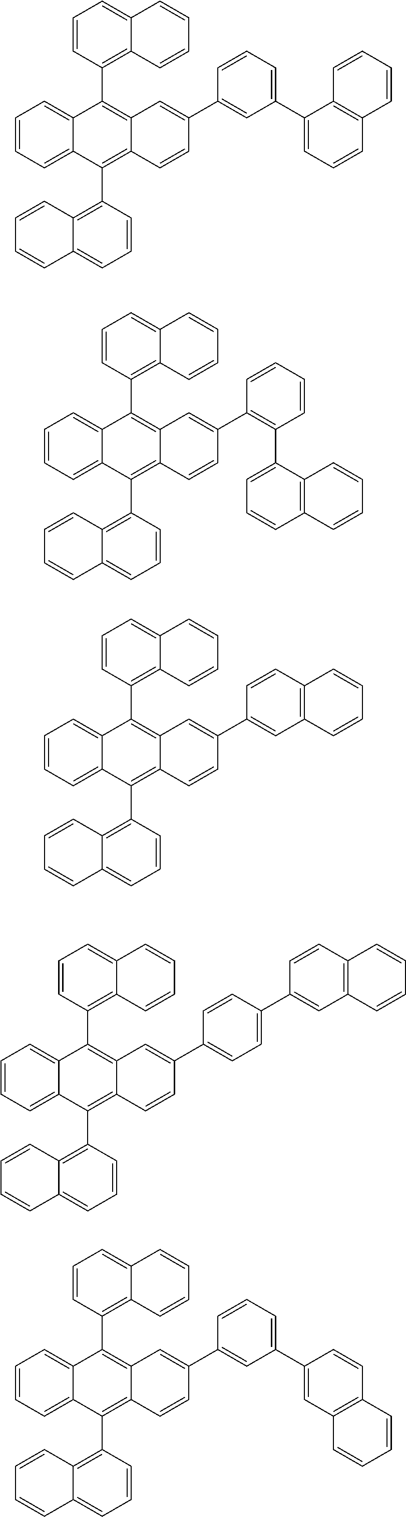

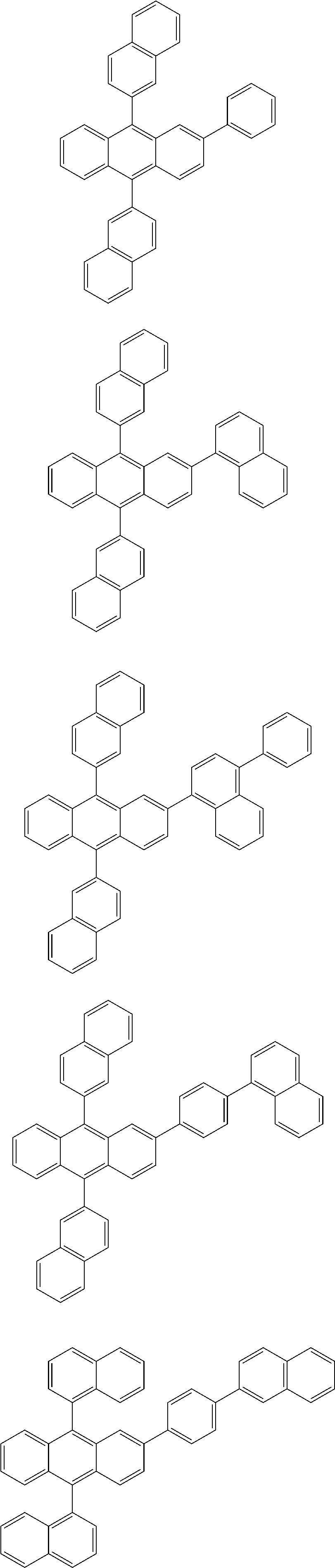

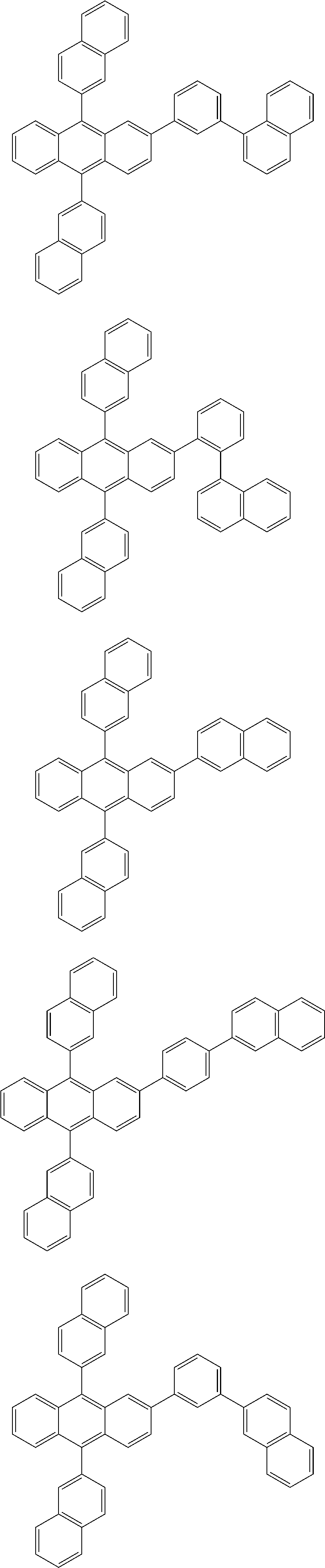

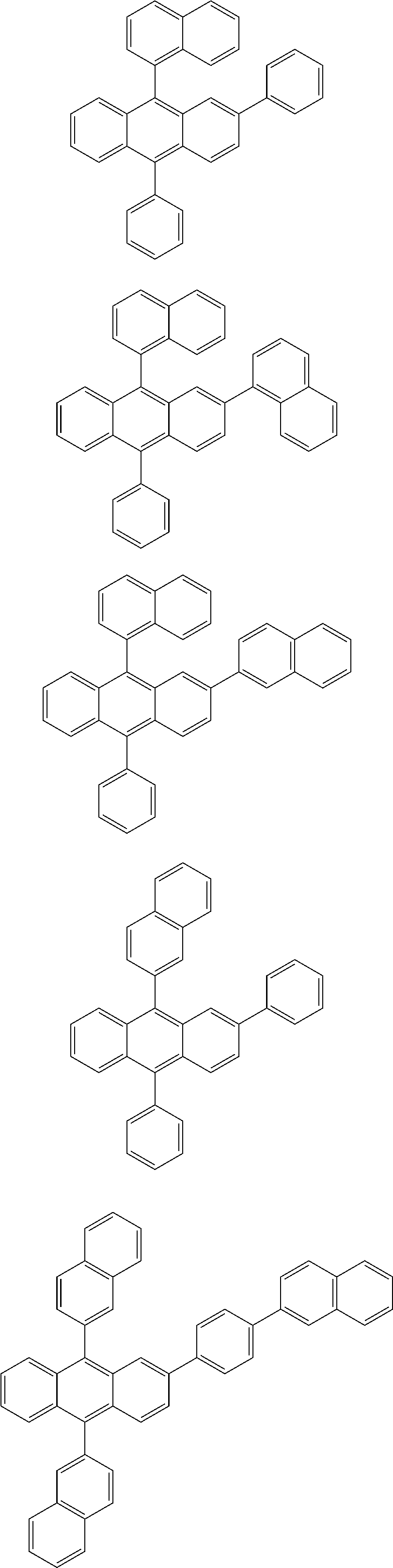

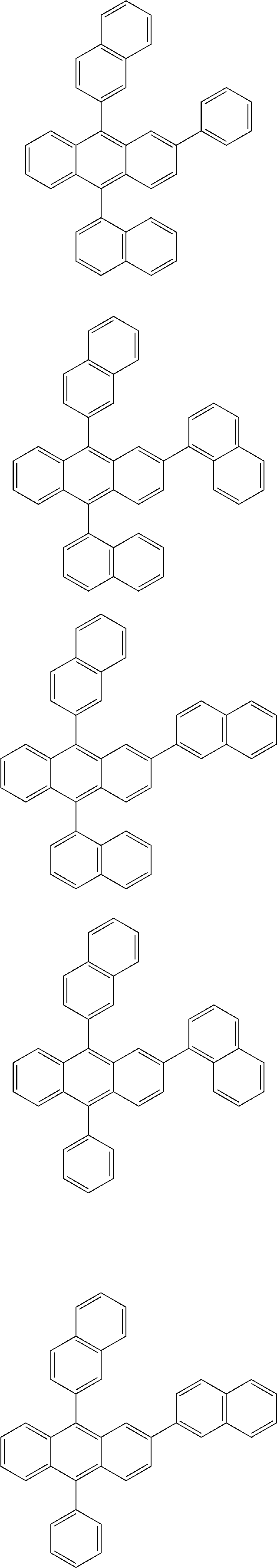

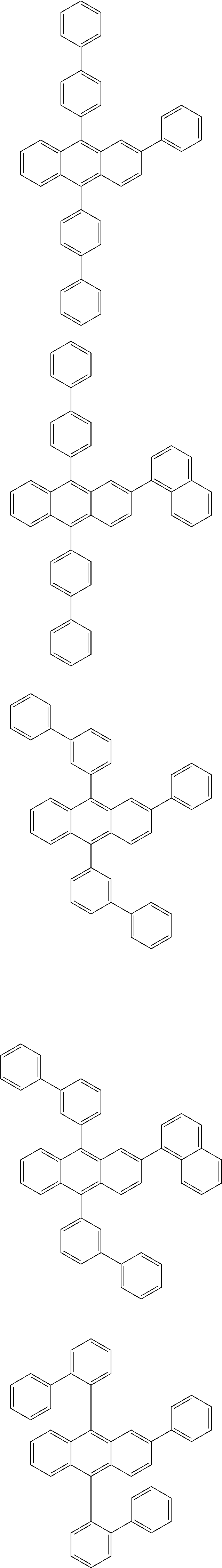

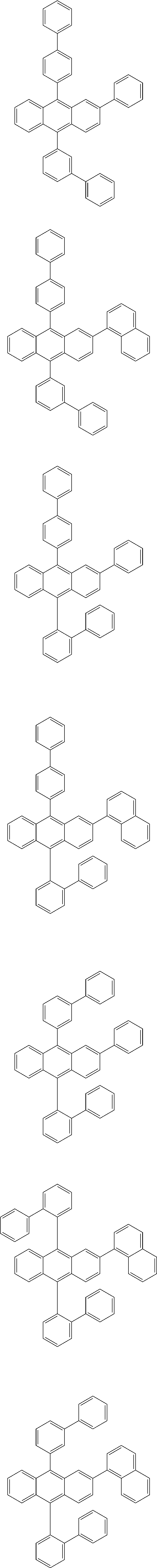

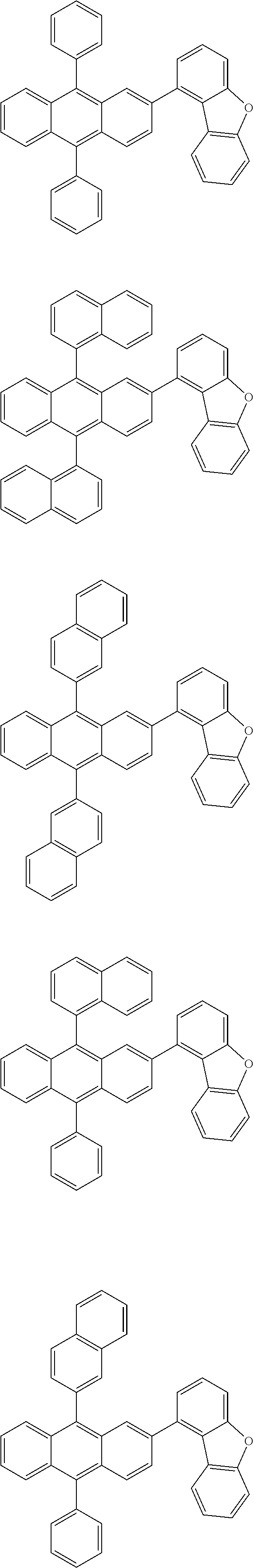 [SMILES: c1ccc(-c2c3ccccc3c(-c3ccccc3)c3cc(-c4cccc5oc6ccccc6c45)ccc23)cc1 ; c1ccc2c(-c3c4ccccc4c(-c4cccc5ccccc45)c4cc(-c5cccc6oc7ccccc7c56)ccc34)cccc2c1 ; c1ccc2cc(-c3c4ccccc4c(-c4ccc5ccccc5c4)c4cc(-c5cccc6oc7ccccc7c56)ccc34)ccc2c1 ; c1ccc(-c2c3ccccc3c(-c3cccc4ccccc34)c3cc(-c4cccc5oc6ccccc6c45)ccc23)cc1 ; c1ccc(-c2c3ccccc3c(-c3ccc4ccccc4c3)c3cc(-c4cccc5oc6ccccc6c45)ccc23)cc1]

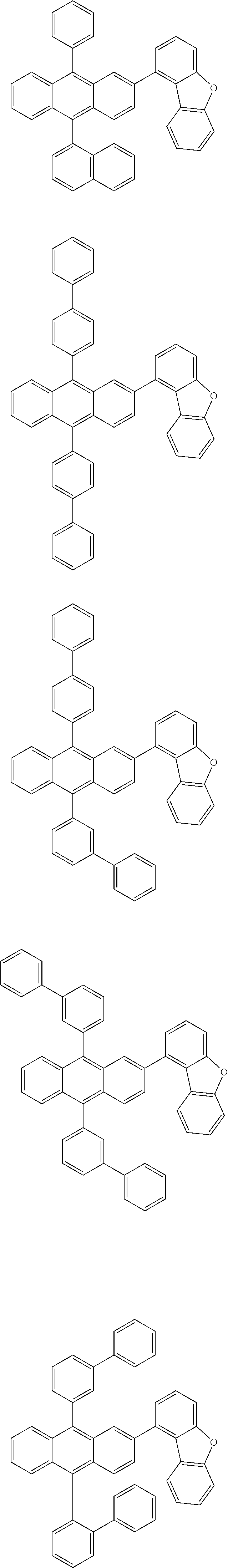 [SMILES: c1ccc(-c2c3ccccc3c(-c3cccc4ccccc34)c3ccc(-c4cccc5oc6ccccc6c45)cc23)cc1 ; c1ccc(-c2ccc(-c3c4ccccc4c(-c4ccc(-c5ccccc5)cc4)c4cc(-c5cccc6oc7ccccc7c56)ccc34)cc2)cc1 ; c1ccc(-c2ccc(-c3c4ccccc4c(-c4cccc(-c5ccccc5)c4)c4ccc(-c5cccc6oc7ccccc7c56)cc34)cc2)cc1 ; c1ccc(-c2cccc(-c3c4ccccc4c(-c4cccc(-c5ccccc5)c4)c4cc(-c5cccc6oc7ccccc7c56)ccc34)c2)cc1 ; c1ccc(-c2cccc(-c3c4ccccc4c(-c4ccccc4-c4ccccc4)c4ccc(-c5cccc6oc7ccccc7c56)cc34)c2)cc1]

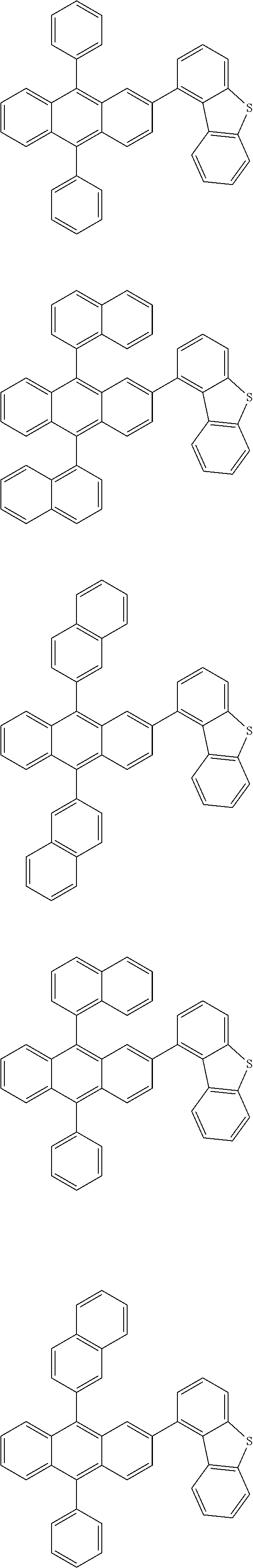 [SMILES: c1ccc(-c2c3ccccc3c(-c3ccccc3)c3cc(-c4cccc5sc6ccccc6c45)ccc23)cc1 ; c1ccc2c(-c3c4ccccc4c(-c4cccc5ccccc45)c4cc(-c5cccc6sc7ccccc7c56)ccc34)cccc2c1 ; c1ccc2cc(-c3c4ccccc4c(-c4ccc5ccccc5c4)c4cc(-c5cccc6sc7ccccc7c56)ccc34)ccc2c1 ; c1ccc(-c2c3ccccc3c(-c3cccc4ccccc34)c3cc(-c4cccc5sc6ccccc6c45)ccc23)cc1 ; c1ccc(-c2c3ccccc3c(-c3ccc4ccccc4c3)c3cc(-c4cccc5sc6ccccc6c45)ccc23)cc1]

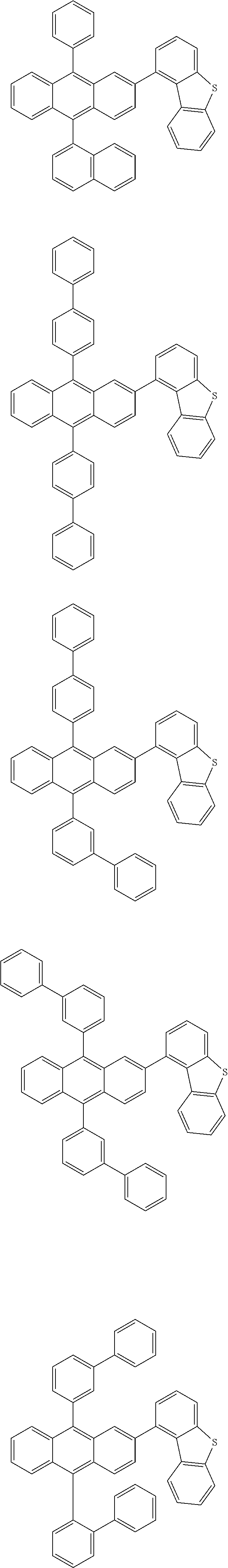 [SMILES: c1ccc(-c2c3ccccc3c(-c3cccc4ccccc34)c3ccc(-c4cccc5sc6ccccc6c45)cc23)cc1 ; c1ccc(-c2ccc(-c3c4ccccc4c(-c4ccc(-c5ccccc5)cc4)c4cc(-c5cccc6sc7ccccc7c56)ccc34)cc2)cc1 ; c1ccc(-c2ccc(-c3c4ccccc4c(-c4cccc(-c5ccccc5)c4)c4ccc(-c5cccc6sc7ccccc7c56)cc34)cc2)cc1 ; c1ccc(-c2cccc(-c3c4ccccc4c(-c4cccc(-c5ccccc5)c4)c4cc(-c5cccc6sc7ccccc7c56)ccc34)c2)cc1 ; c1ccc(-c2cccc(-c3c4ccccc4c(-c4ccccc4-c4ccccc4)c4ccc(-c5cccc6sc7ccccc7c56)cc34)c2)cc1]

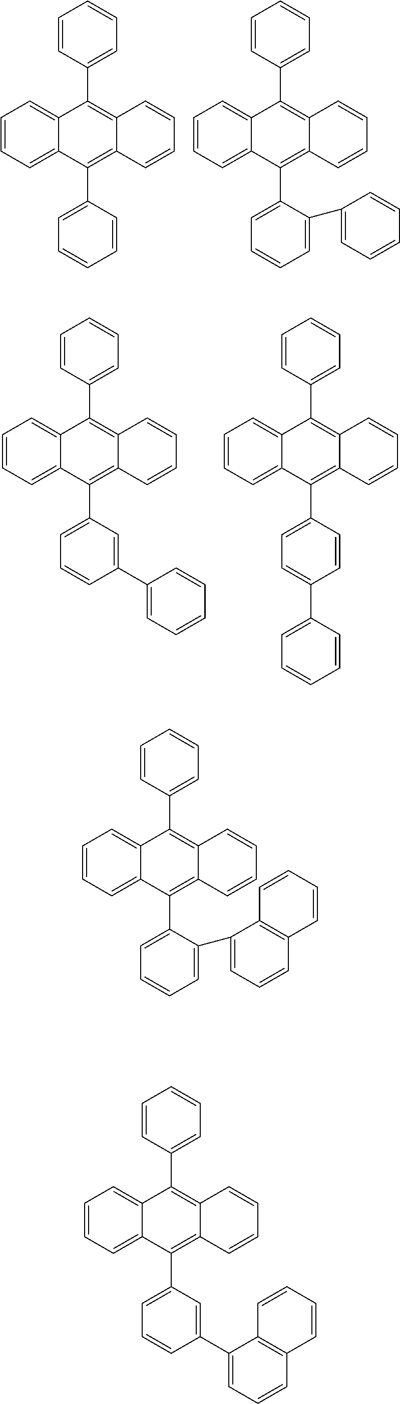

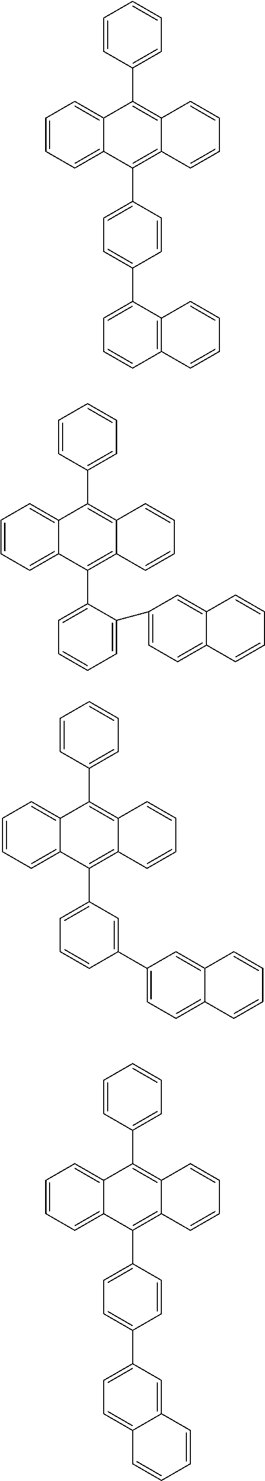

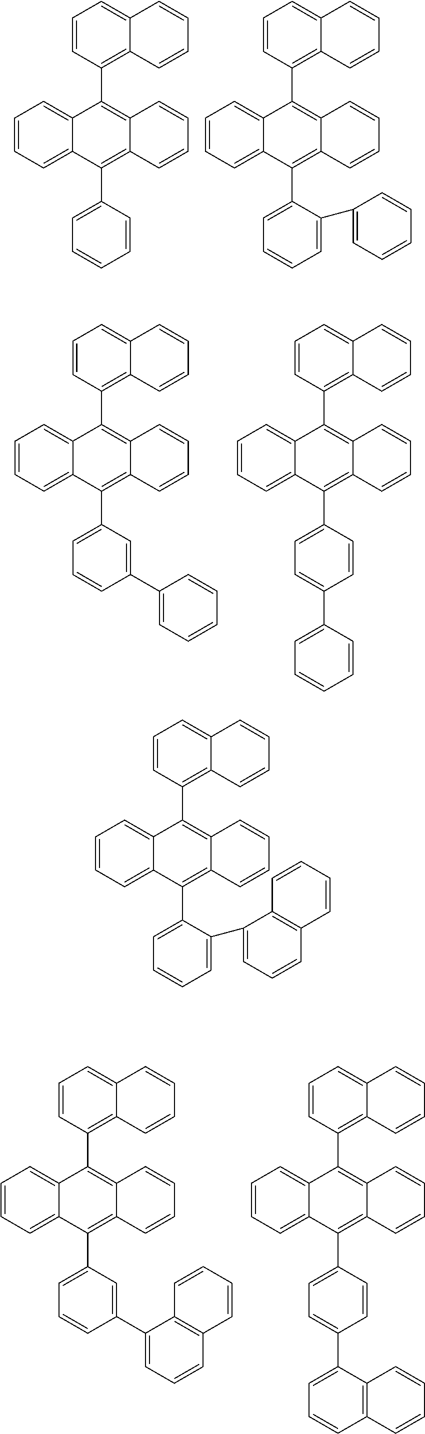

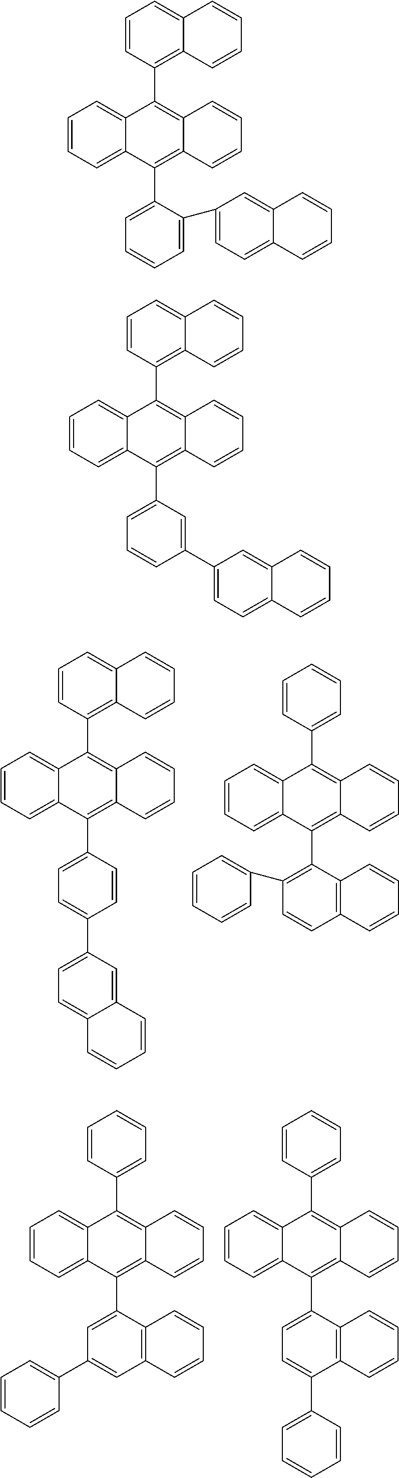

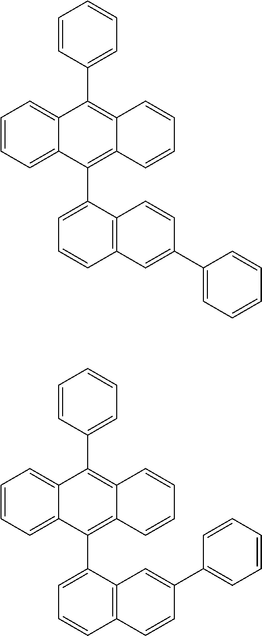

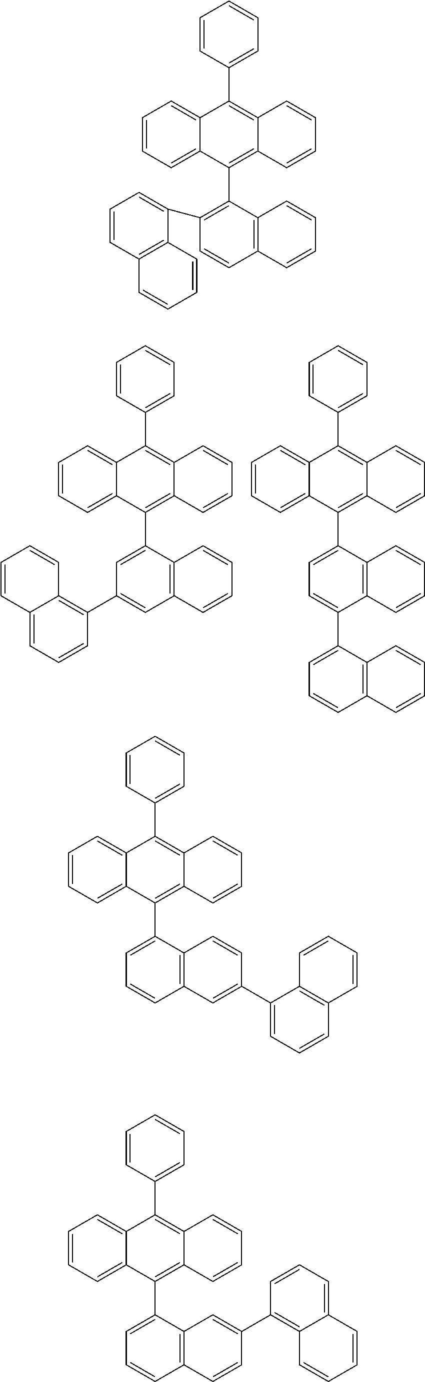

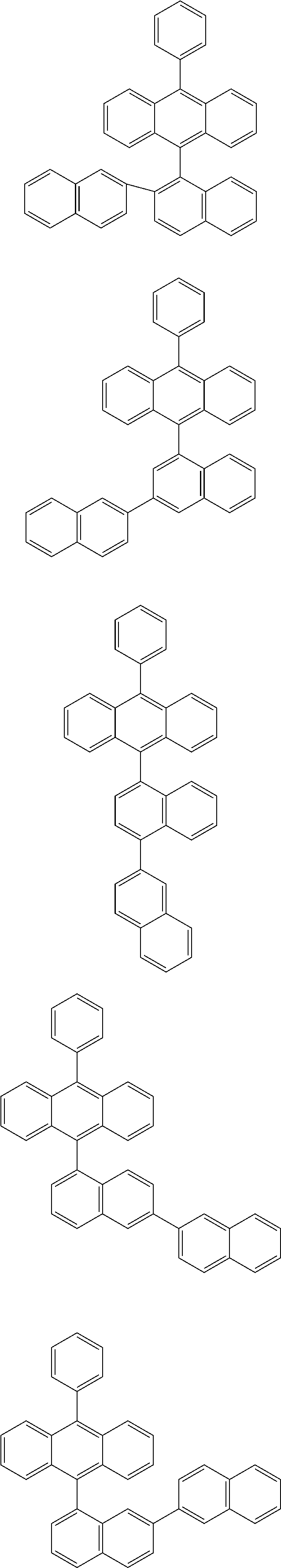

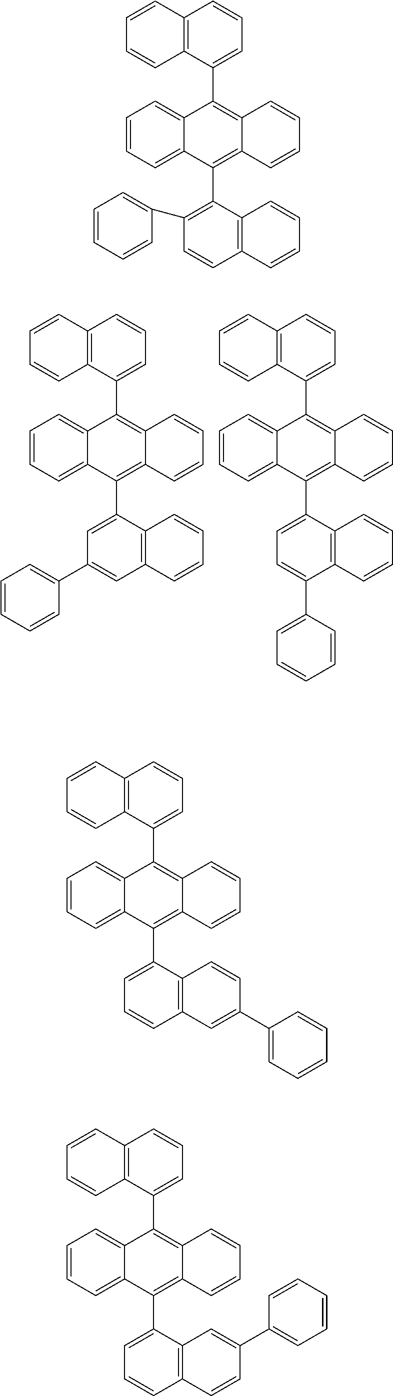

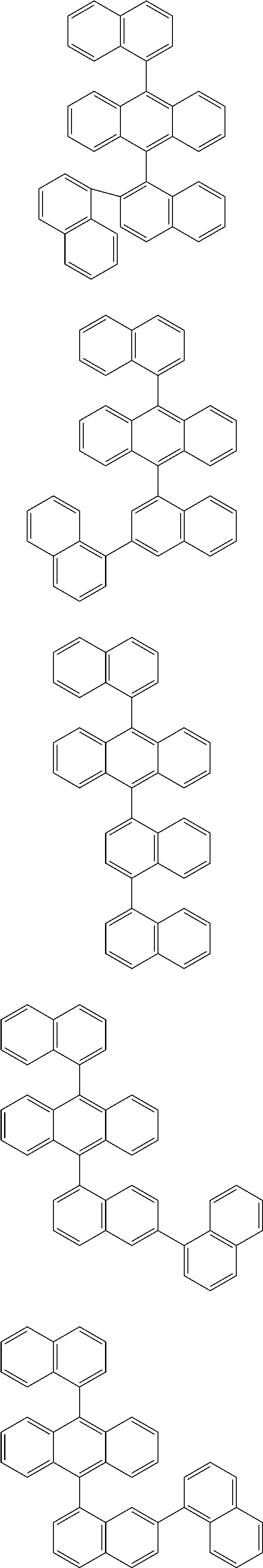

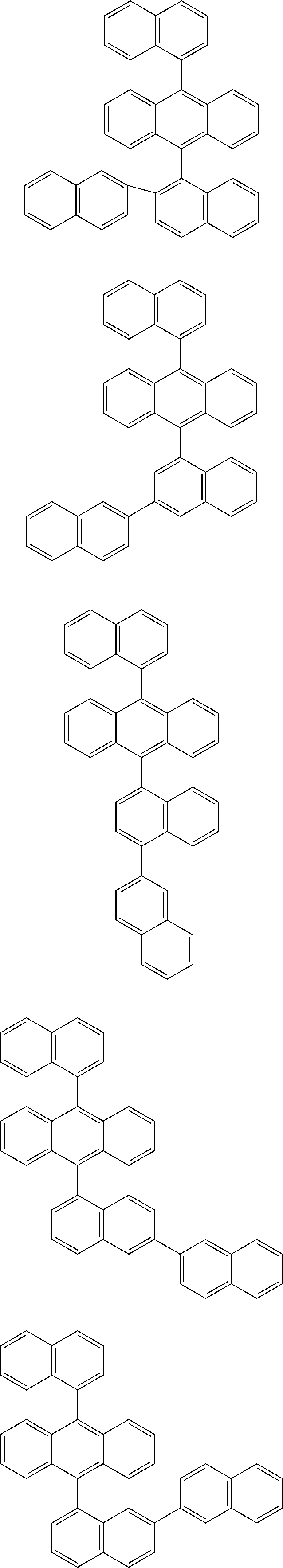

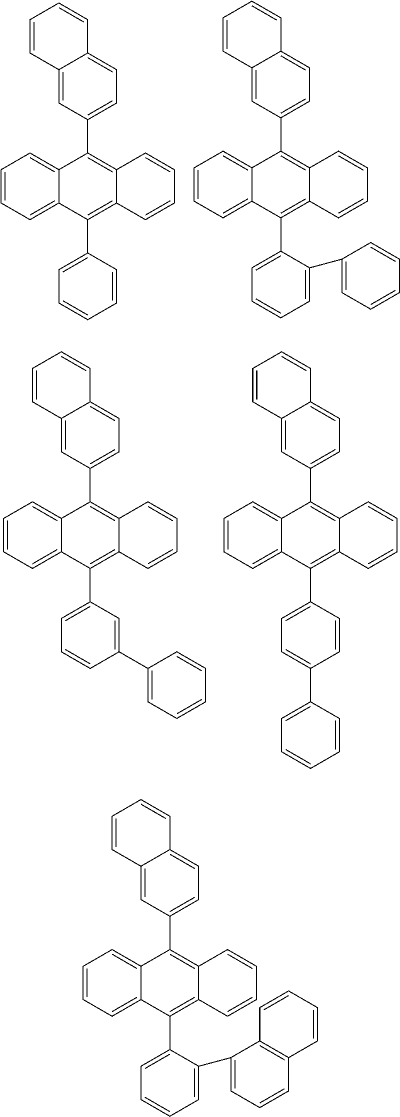

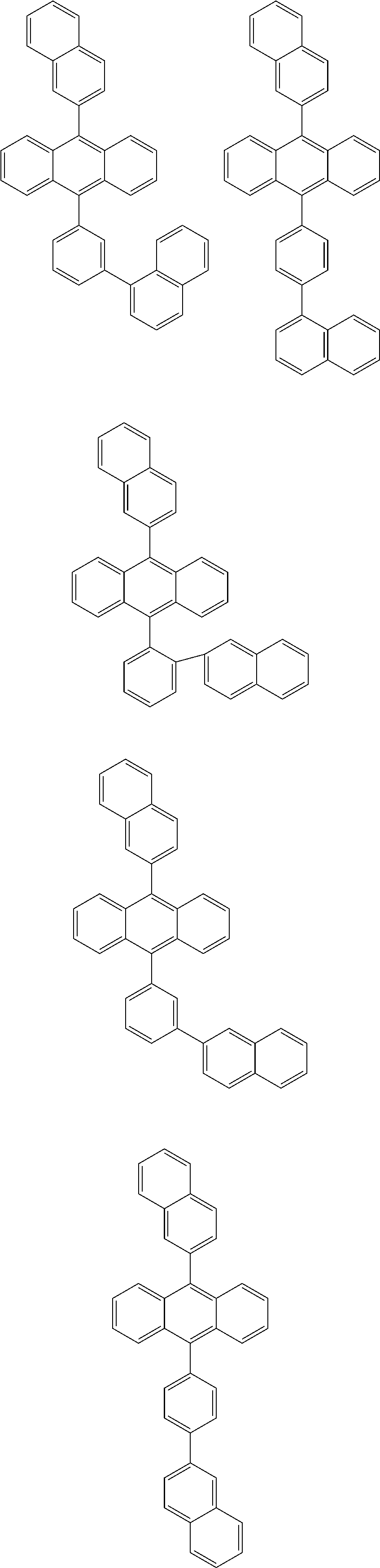

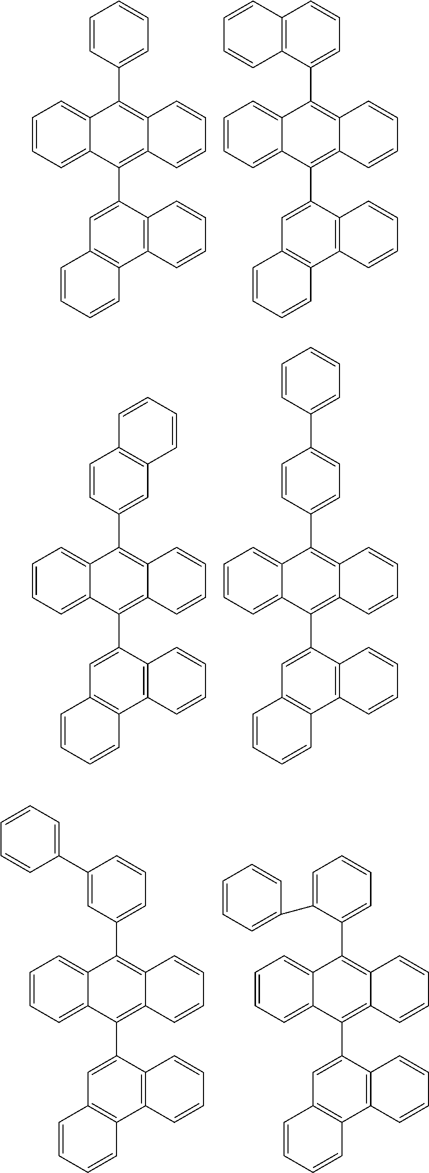

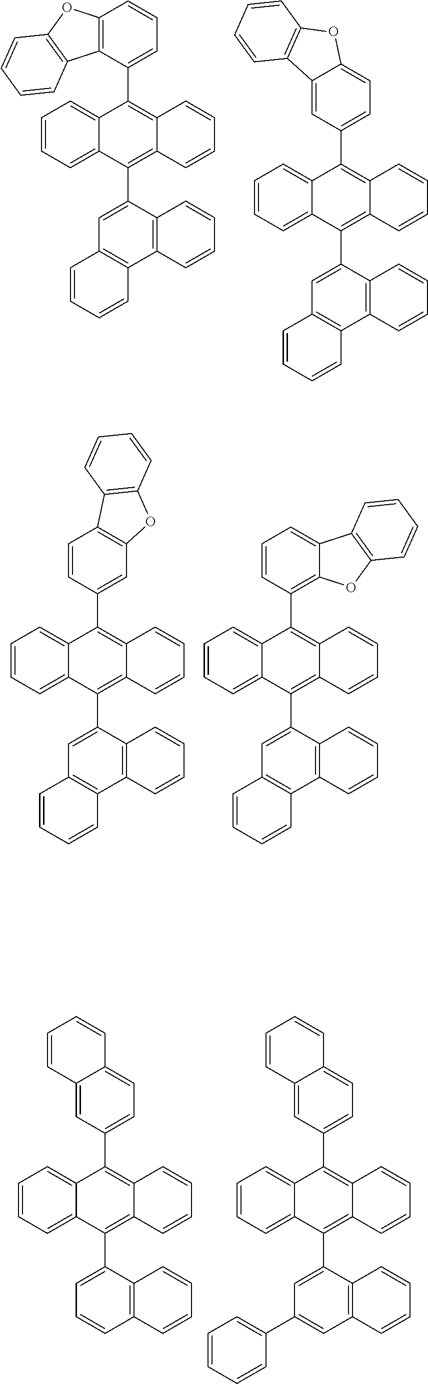

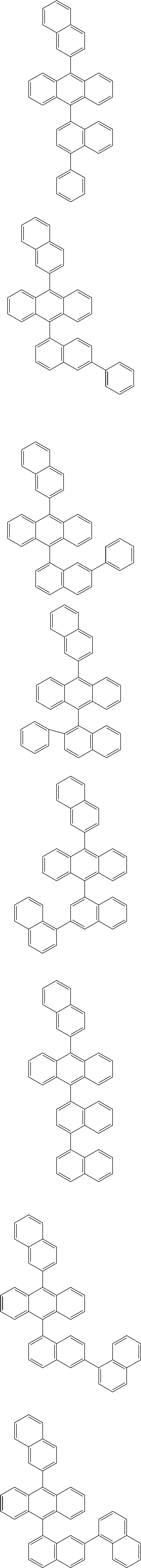

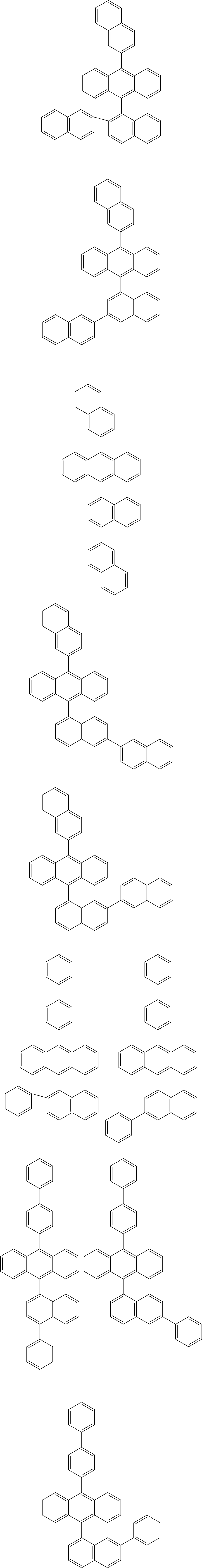

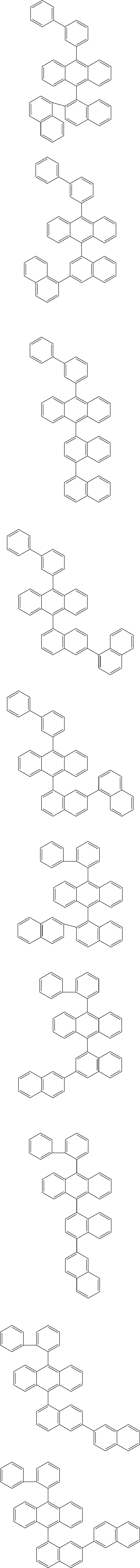

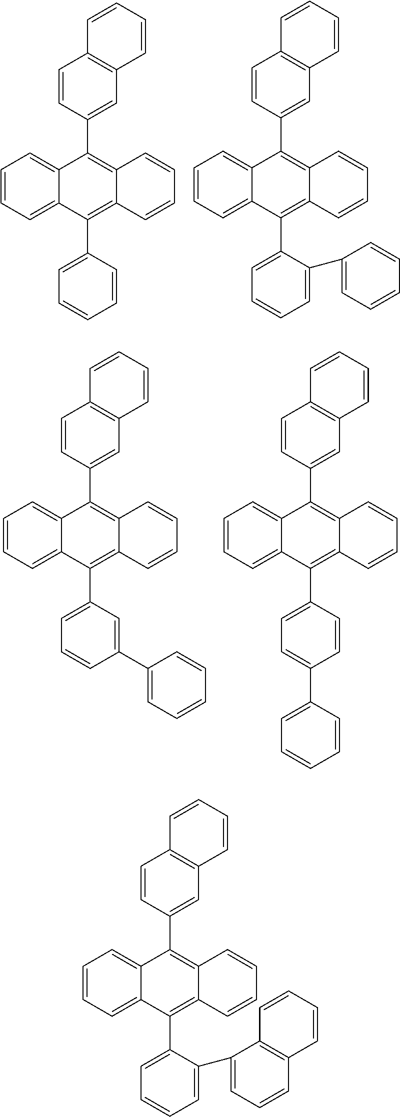

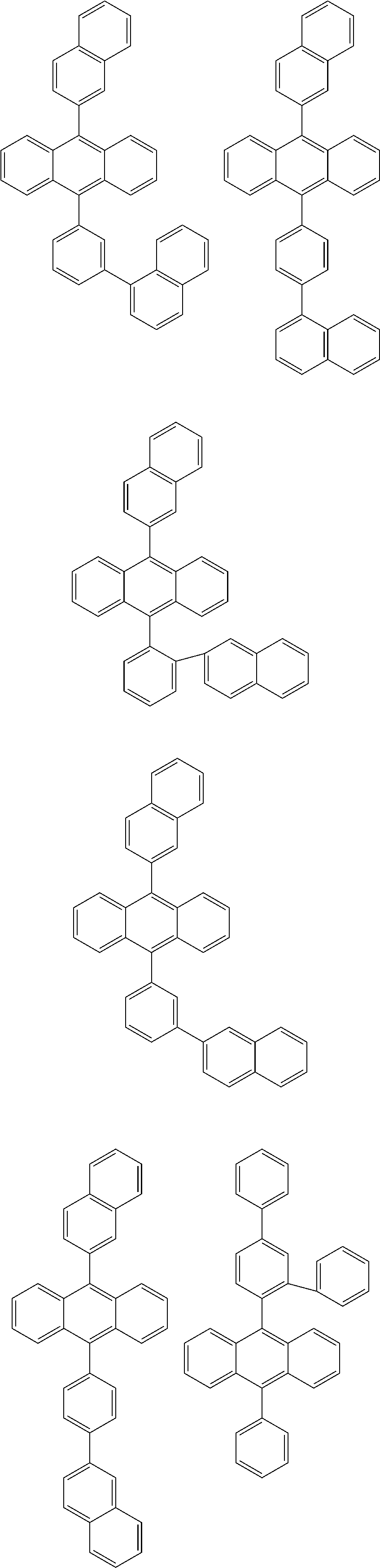

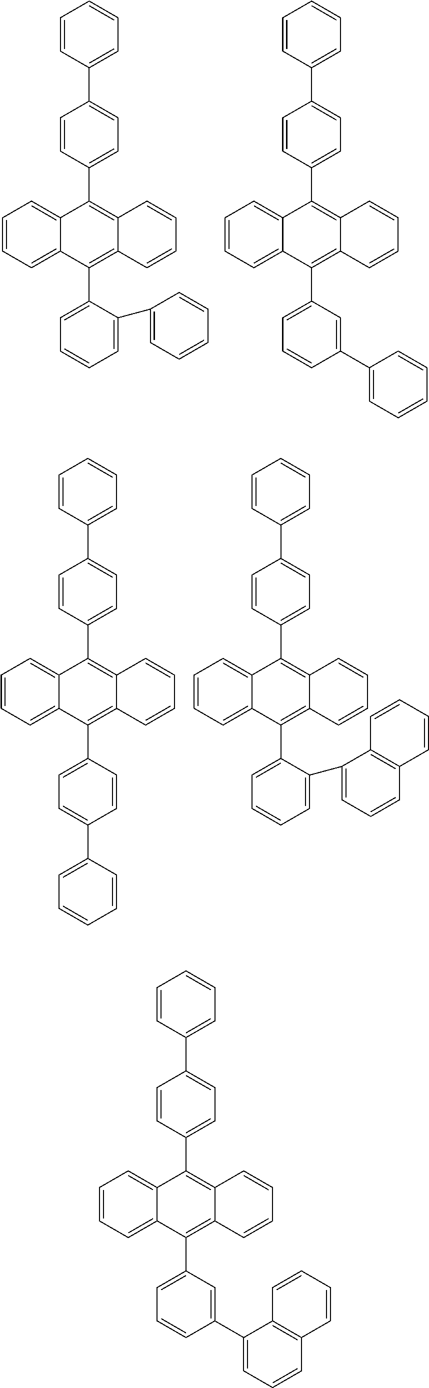

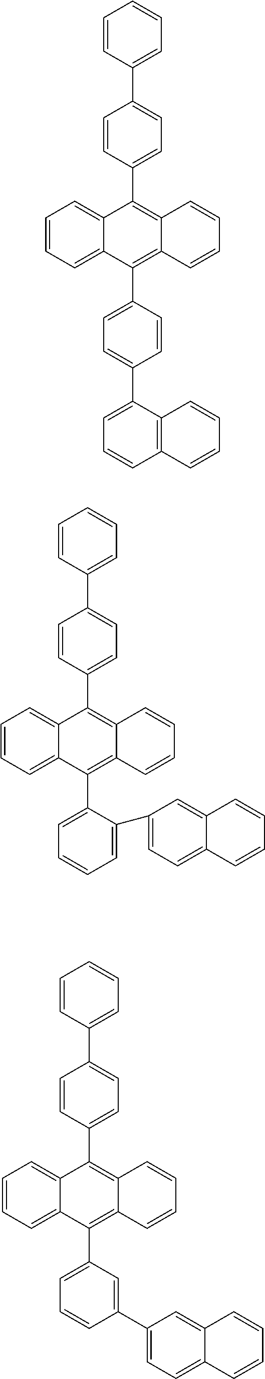

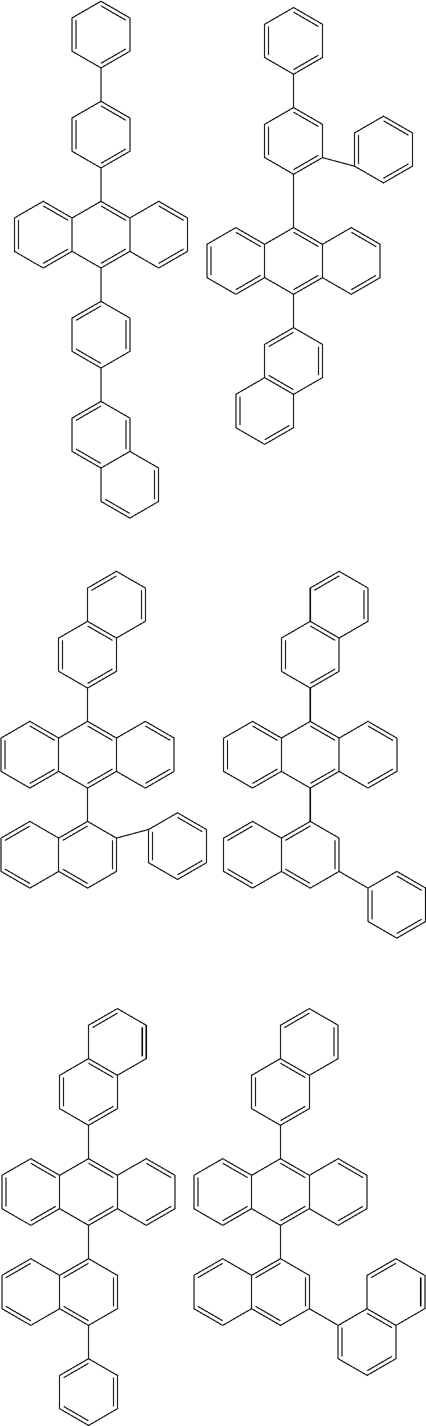

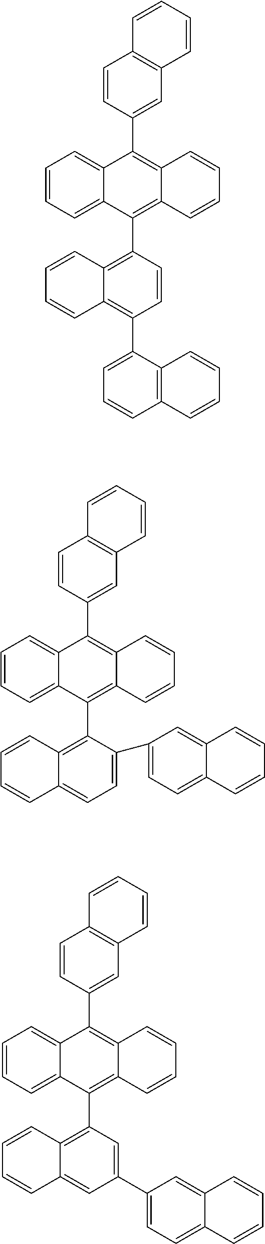

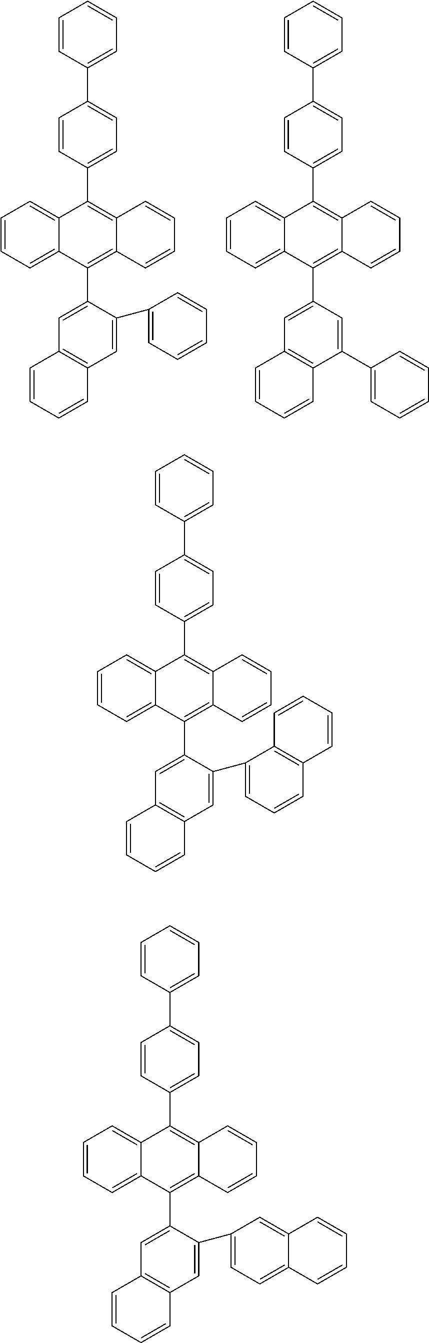

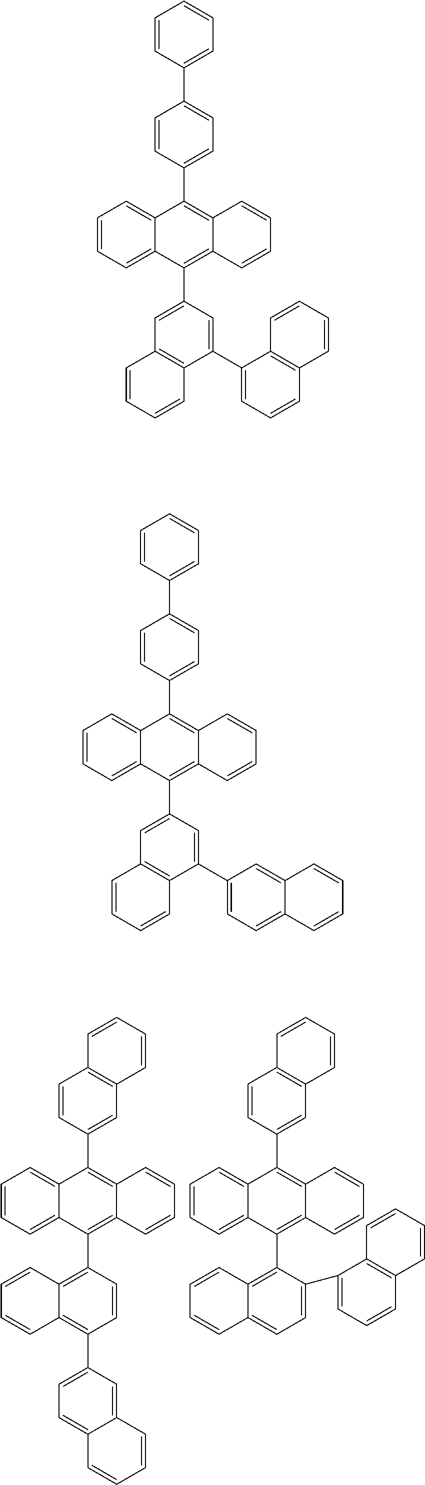

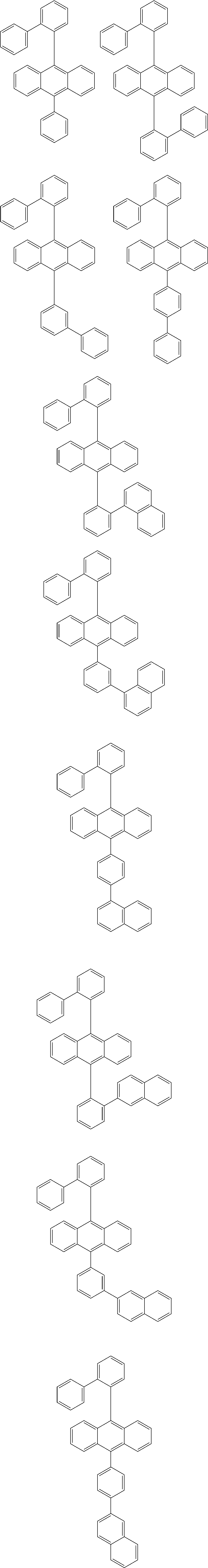

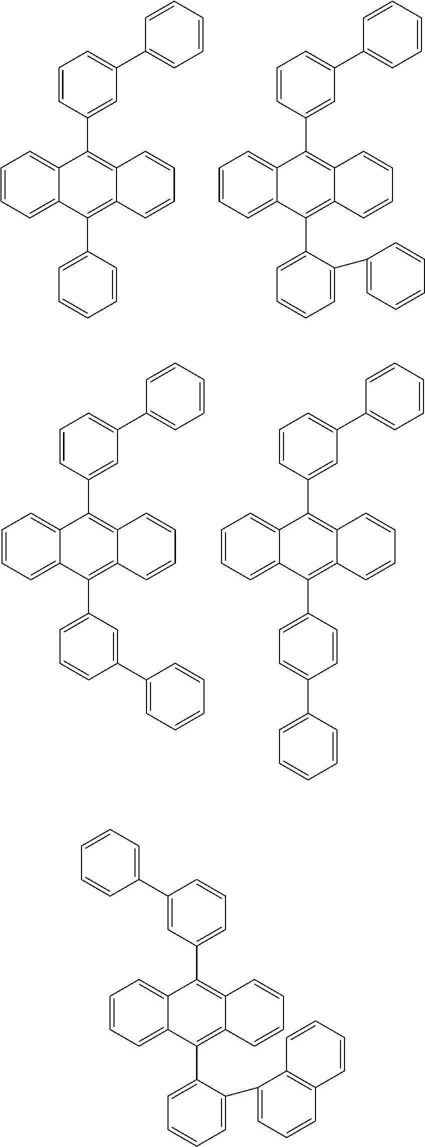

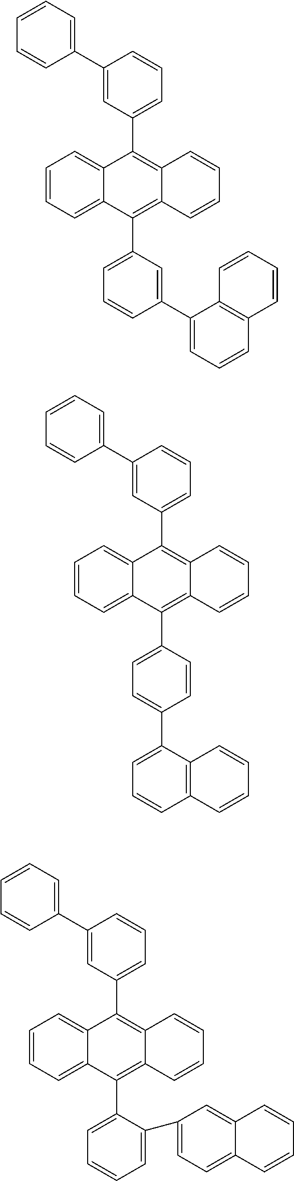

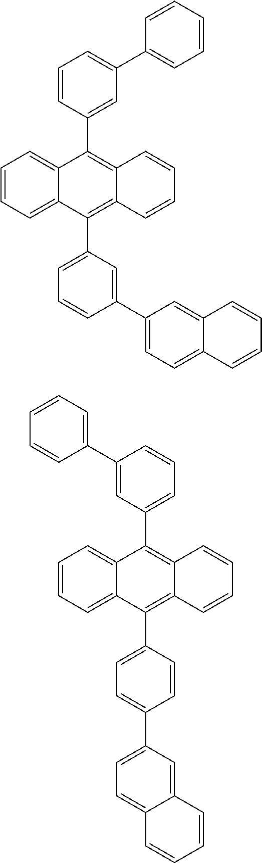

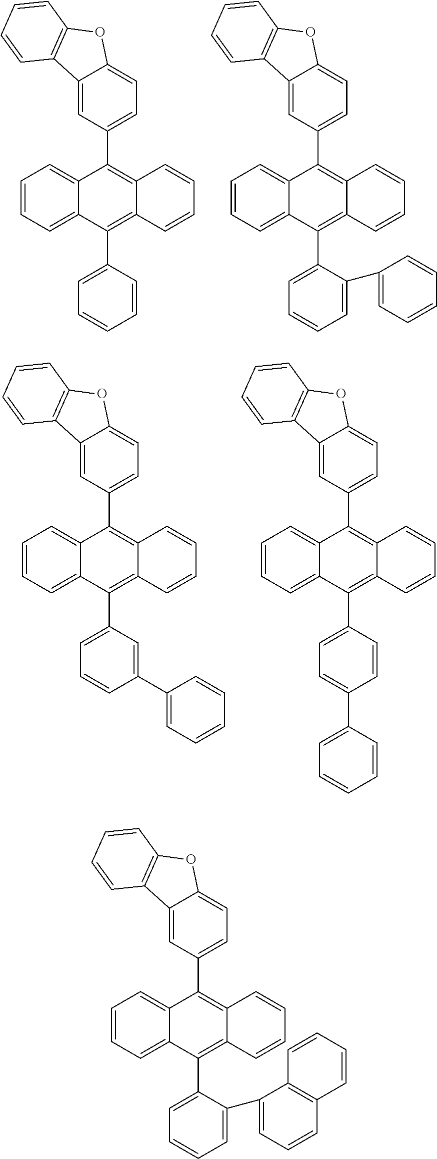

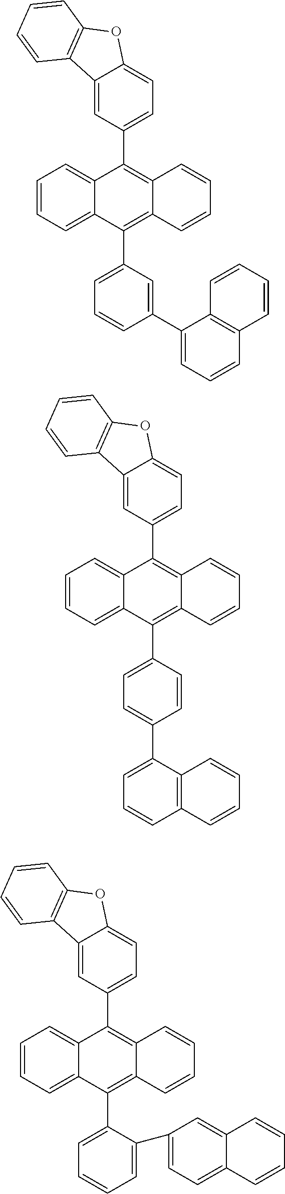 [SMILES: c1cc(-c2cccc3ccccc23)cc(-c2c3ccccc3c(-c3ccc4oc5ccccc5c4c3)c3ccccc23)c1 ; c1ccc2c(-c3ccc(-c4c5ccccc5c(-c5ccc6oc7ccccc7c6c5)c5ccccc45)cc3)cccc2c1 ; c1ccc(-c2c3ccccc3c(-c3ccc4oc5ccccc5c4c3)c3ccccc23)c(-c2ccc3ccccc3c2)c1]

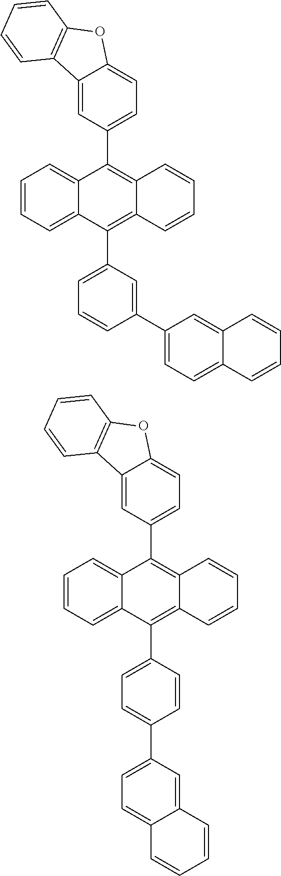 [SMILES: c1cc(-c2ccc3ccccc3c2)cc(-c2c3ccccc3c(-c3ccc4oc5ccccc5c4c3)c3ccccc23)c1 ; c1ccc2cc(-c3ccc(-c4c5ccccc5c(-c5ccc6oc7ccccc7c6c5)c5ccccc45)cc3)ccc2c1]

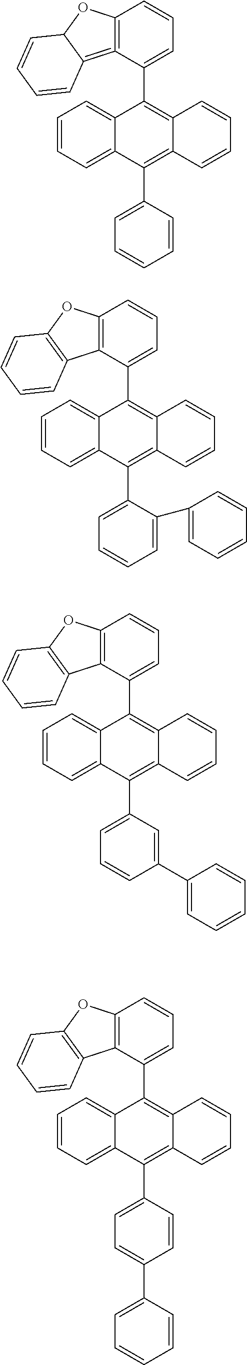

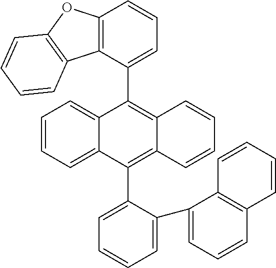 [SMILES: c1ccc(-c2c3ccccc3c(-c3cccc4oc5ccccc5c34)c3ccccc23)c(-c2cccc3ccccc23)c1]

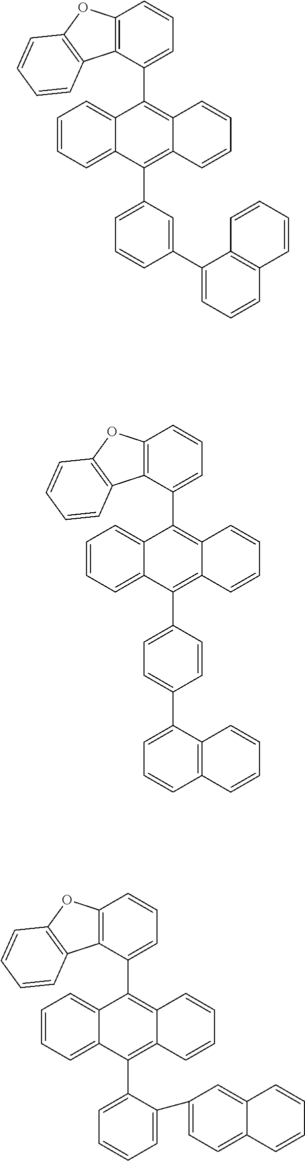 [SMILES: c1cc(-c2cccc3ccccc23)cc(-c2c3ccccc3c(-c3cccc4oc5ccccc5c34)c3ccccc23)c1 ; c1ccc2c(-c3ccc(-c4c5ccccc5c(-c5cccc6oc7ccccc7c56)c5ccccc45)cc3)cccc2c1 ; c1ccc(-c2c3ccccc3c(-c3cccc4oc5ccccc5c34)c3ccccc23)c(-c2ccc3ccccc3c2)c1]

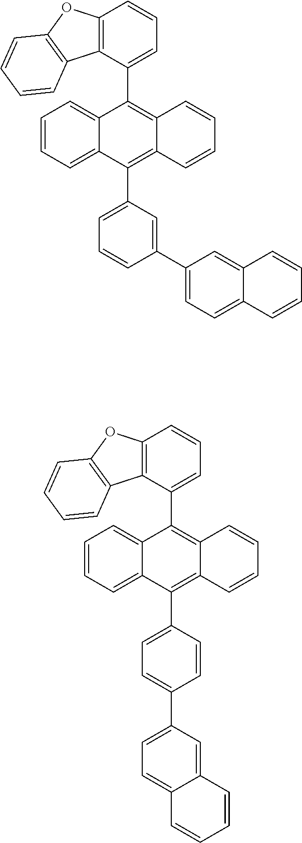 [SMILES: c1cc(-c2ccc3ccccc3c2)cc(-c2c3ccccc3c(-c3cccc4oc5ccccc5c34)c3ccccc23)c1 ; c1ccc2cc(-c3ccc(-c4c5ccccc5c(-c5cccc6oc7ccccc7c56)c5ccccc45)cc3)ccc2c1]

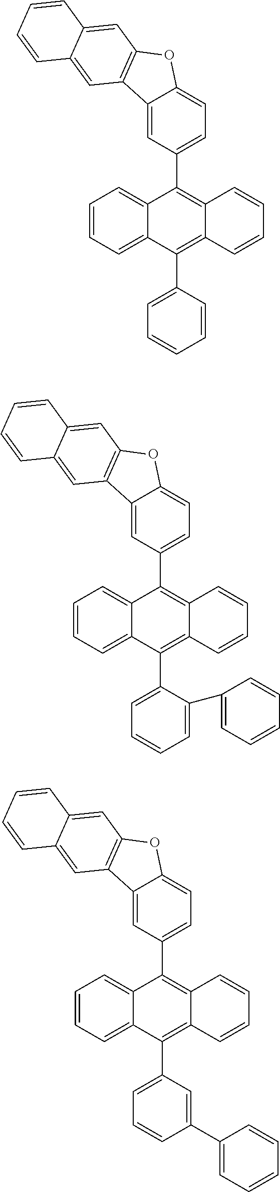 [SMILES: c1ccc(-c2c3ccccc3c(-c3ccc4oc5cc6ccccc6cc5c4c3)c3ccccc23)cc1 ; c1ccc(-c2ccccc2-c2c3ccccc3c(-c3ccc4oc5cc6ccccc6cc5c4c3)c3ccccc23)cc1 ; c1ccc(-c2cccc(-c3c4ccccc4c(-c4ccc5oc6cc7ccccc7cc6c5c4)c4ccccc34)c2)cc1]

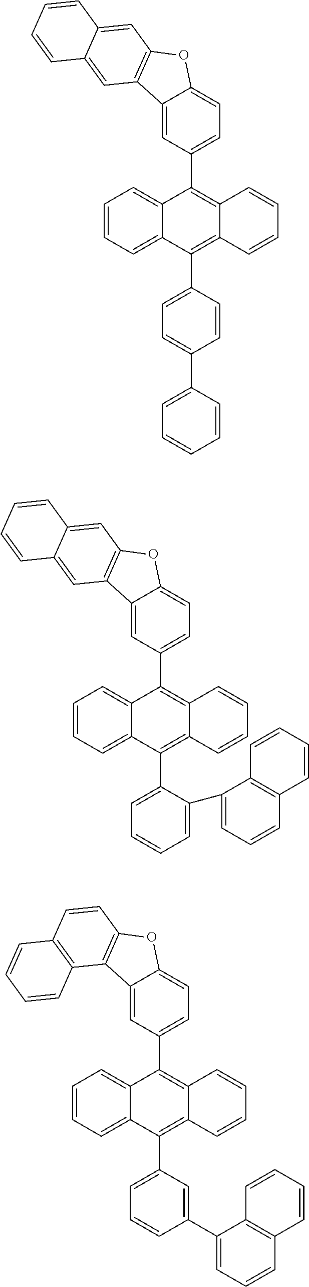 [SMILES: c1ccc(-c2ccc(-c3c4ccccc4c(-c4ccc5oc6cc7ccccc7cc6c5c4)c4ccccc34)cc2)cc1 ; c1ccc(-c2c3ccccc3c(-c3ccc4oc5cc6ccccc6cc5c4c3)c3ccccc23)c(-c2cccc3ccccc23)c1 ; c1cc(-c2cccc3ccccc23)cc(-c2c3ccccc3c(-c3ccc4oc5ccc6ccccc6c5c4c3)c3ccccc23)c1]

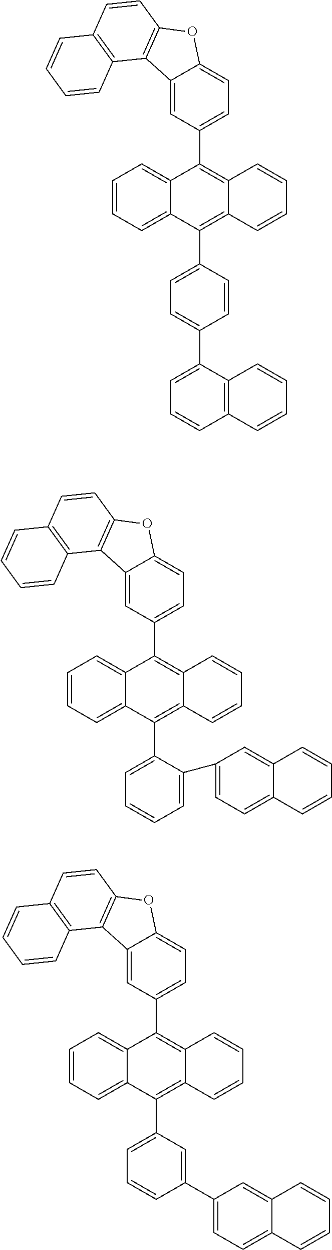 [SMILES: c1ccc2c(-c3ccc(-c4c5ccccc5c(-c5ccc6oc7ccc8ccccc8c7c6c5)c5ccccc45)cc3)cccc2c1 ; c1ccc(-c2c3ccccc3c(-c3ccc4oc5ccc6ccccc6c5c4c3)c3ccccc23)c(-c2ccc3ccccc3c2)c1 ; c1cc(-c2ccc3ccccc3c2)cc(-c2c3ccccc3c(-c3ccc4oc5ccc6ccccc6c5c4c3)c3ccccc23)c1]

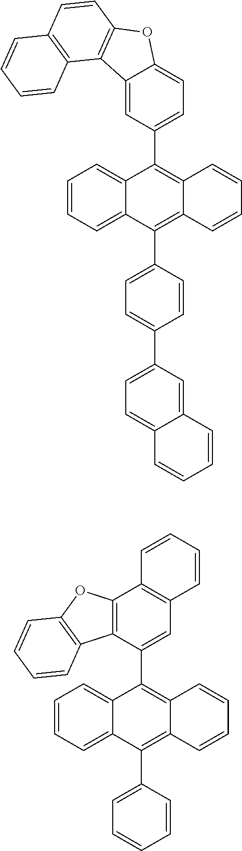 [SMILES: c1ccc2cc(-c3ccc(-c4c5ccccc5c(-c5ccc6oc7ccc8ccccc8c7c6c5)c5ccccc45)cc3)ccc2c1 ; c1ccc(-c2c3ccccc3c(-c3cc4ccccc4c4oc5ccccc5c34)c3ccccc23)cc1]

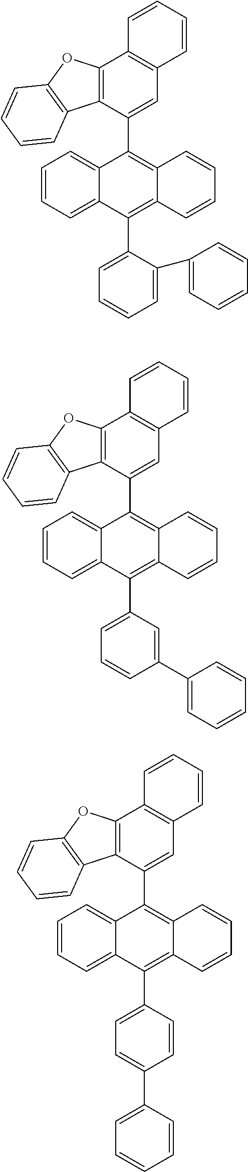

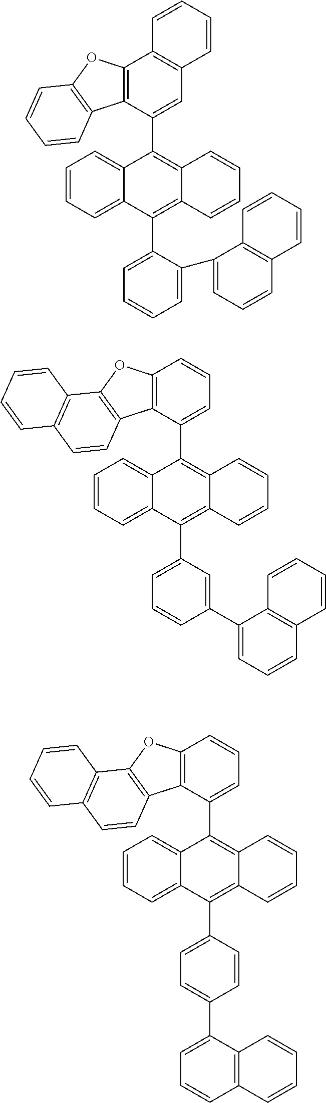 [SMILES: c1ccc(-c2c3ccccc3c(-c3cc4ccccc4c4oc5ccccc5c34)c3ccccc23)c(-c2cccc3ccccc23)c1 ; c1cc(-c2cccc3ccccc23)cc(-c2c3ccccc3c(-c3cccc4oc5c6ccccc6ccc5c34)c3ccccc23)c1 ; c1ccc2c(-c3ccc(-c4c5ccccc5c(-c5cccc6oc7c8ccccc8ccc7c56)c5ccccc45)cc3)cccc2c1]

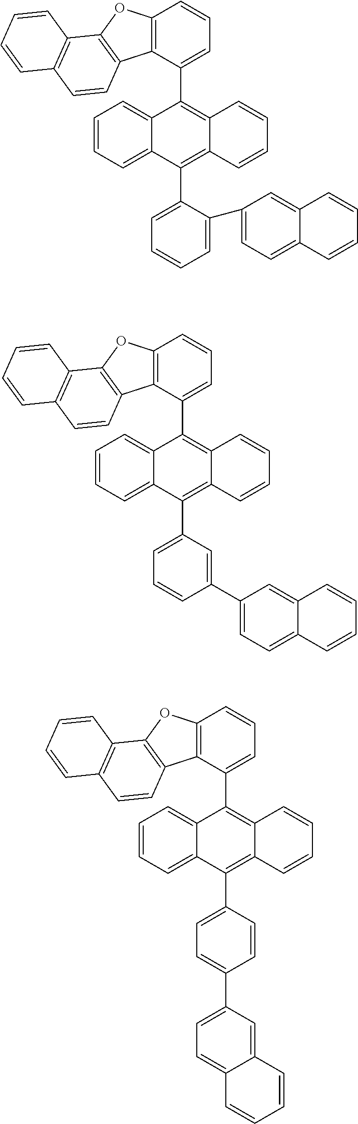 [SMILES: c1ccc(-c2c3ccccc3c(-c3cccc4oc5c6ccccc6ccc5c34)c3ccccc23)c(-c2ccc3ccccc3c2)c1 ; c1cc(-c2ccc3ccccc3c2)cc(-c2c3ccccc3c(-c3cccc4oc5c6ccccc6ccc5c34)c3ccccc23)c1 ; c1ccc2cc(-c3ccc(-c4c5ccccc5c(-c5cccc6oc7c8ccccc8ccc7c56)c5ccccc45)cc3)ccc2c1]

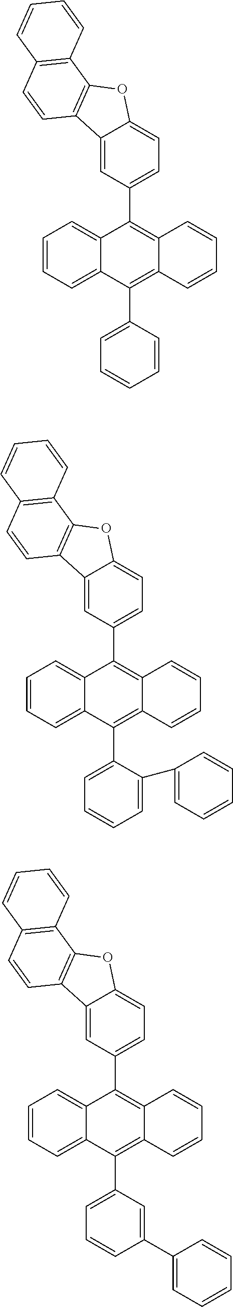 [SMILES: c1ccc(-c2c3ccccc3c(-c3ccc4oc5c6ccccc6ccc5c4c3)c3ccccc23)cc1 ; c1ccc(-c2ccccc2-c2c3ccccc3c(-c3ccc4oc5c6ccccc6ccc5c4c3)c3ccccc23)cc1 ; c1ccc(-c2cccc(-c3c4ccccc4c(-c4ccc5oc6c7ccccc7ccc6c5c4)c4ccccc34)c2)cc1]

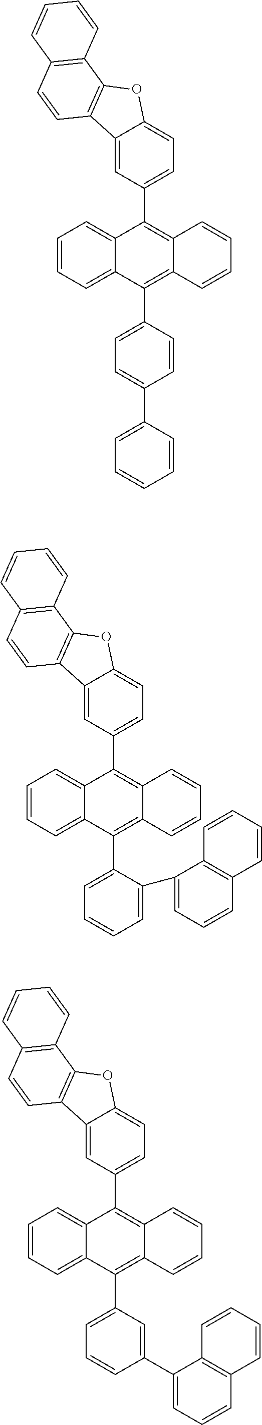 [SMILES: c1ccc(-c2ccc(-c3c4ccccc4c(-c4ccc5oc6c7ccccc7ccc6c5c4)c4ccccc34)cc2)cc1 ; c1ccc(-c2c3ccccc3c(-c3ccc4oc5c6ccccc6ccc5c4c3)c3ccccc23)c(-c2cccc3ccccc23)c1 ; c1cc(-c2cccc3ccccc23)cc(-c2c3ccccc3c(-c3ccc4oc5c6ccccc6ccc5c4c3)c3ccccc23)c1]

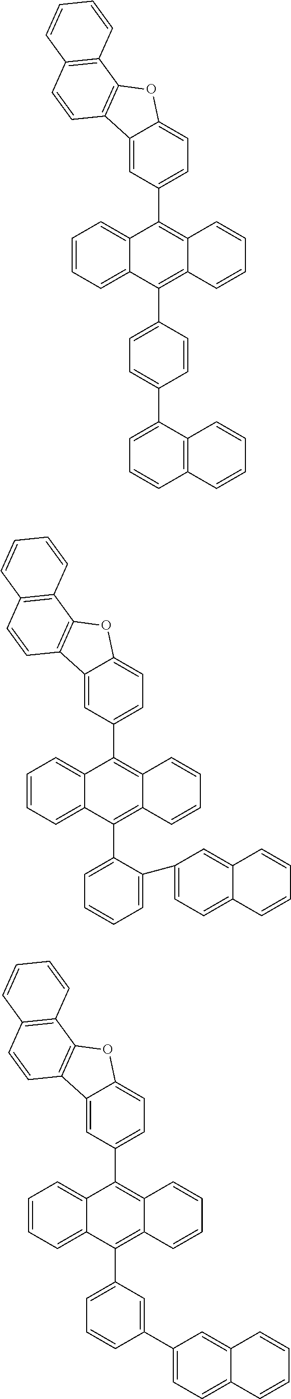 [SMILES: c1ccc2c(-c3ccc(-c4c5ccccc5c(-c5ccc6oc7c8ccccc8ccc7c6c5)c5ccccc45)cc3)cccc2c1 ; c1ccc(-c2c3ccccc3c(-c3ccc4oc5c6ccccc6ccc5c4c3)c3ccccc23)c(-c2ccc3ccccc3c2)c1 ; c1cc(-c2ccc3ccccc3c2)cc(-c2c3ccccc3c(-c3ccc4oc5c6ccccc6ccc5c4c3)c3ccccc23)c1]

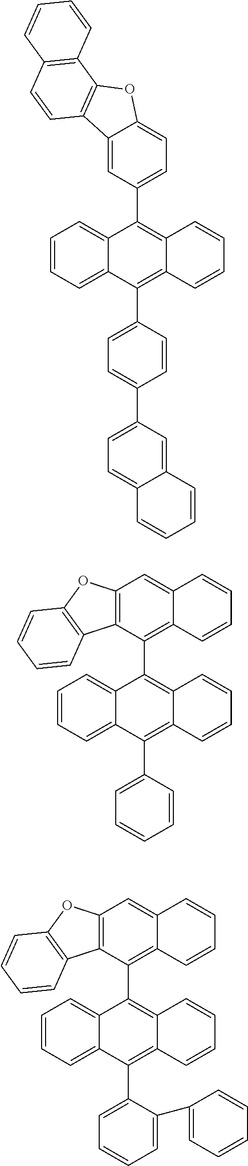

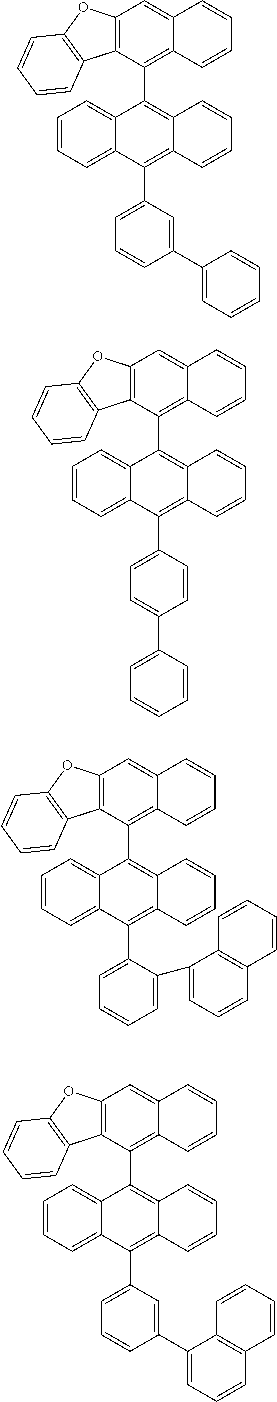

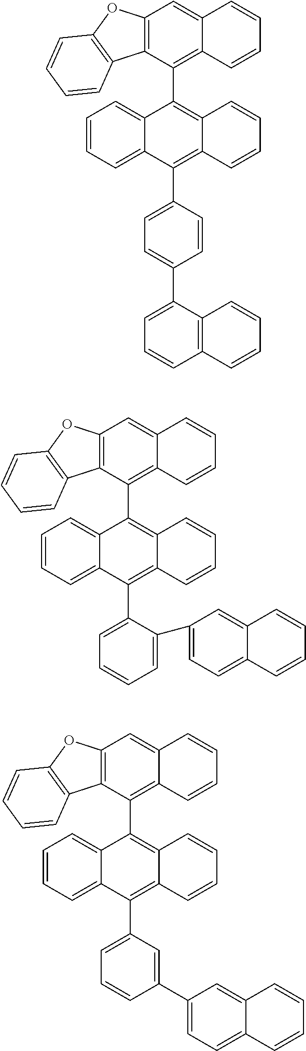 [SMILES: c1ccc2c(-c3ccc(-c4c5ccccc5c(-c5c6ccccc6cc6oc7ccccc7c56)c5ccccc45)cc3)cccc2c1 ; c1ccc(-c2c3ccccc3c(-c3c4ccccc4cc4oc5ccccc5c34)c3ccccc23)c(-c2ccc3ccccc3c2)c1 ; c1cc(-c2ccc3ccccc3c2)cc(-c2c3ccccc3c(-c3c4ccccc4cc4oc5ccccc5c34)c3ccccc23)c1]

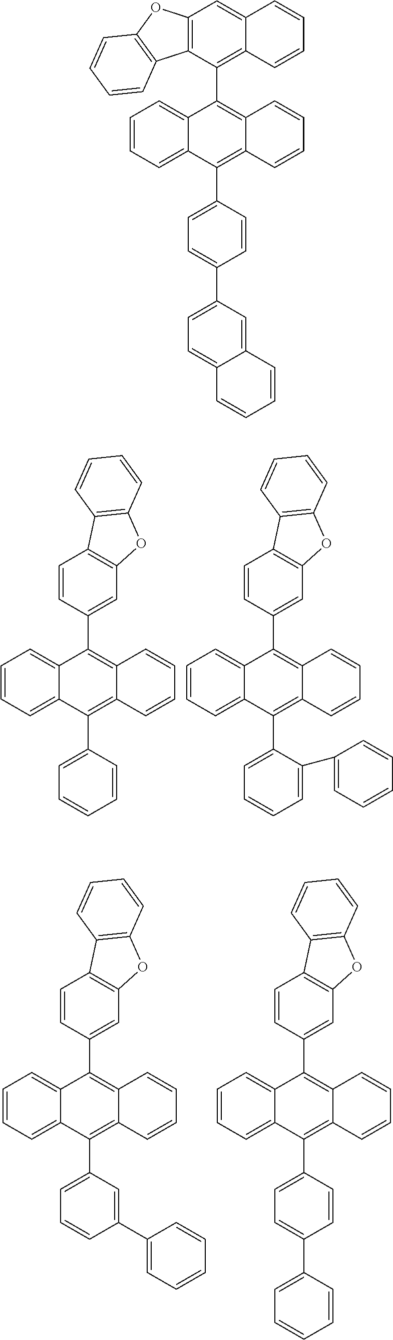

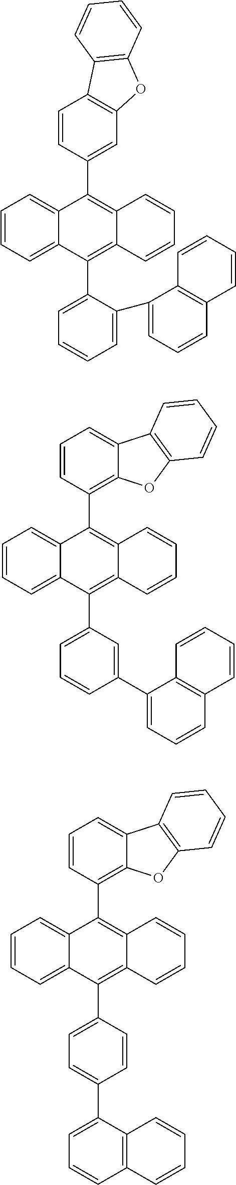 [SMILES: c1ccc(-c2c3ccccc3c(-c3ccc4c(c3)oc3ccccc34)c3ccccc23)c(-c2cccc3ccccc23)c1 ; c1cc(-c2cccc3ccccc23)cc(-c2c3ccccc3c(-c3cccc4c3oc3ccccc34)c3ccccc23)c1 ; c1ccc2c(-c3ccc(-c4c5ccccc5c(-c5cccc6c5oc5ccccc56)c5ccccc45)cc3)cccc2c1]

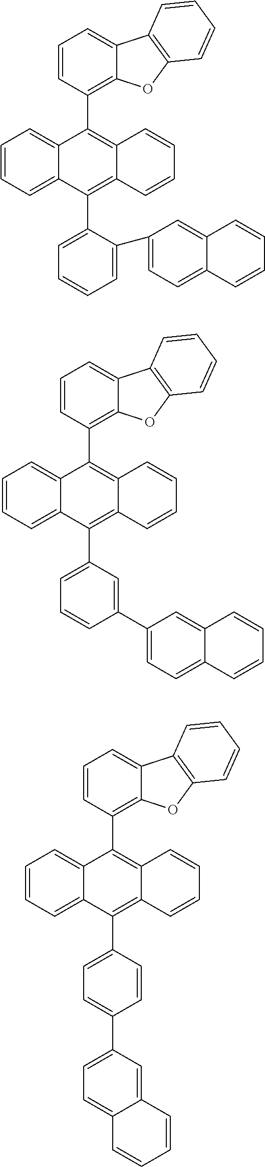

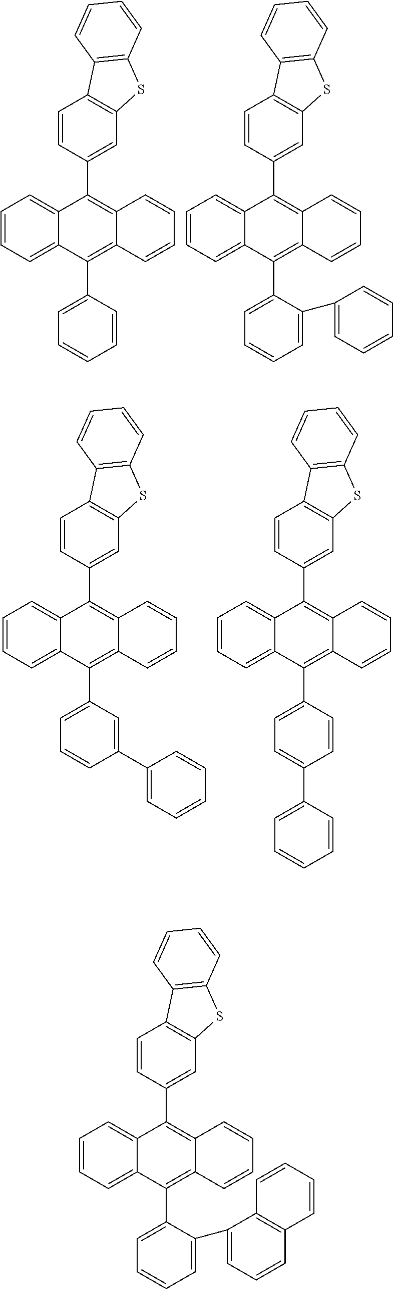

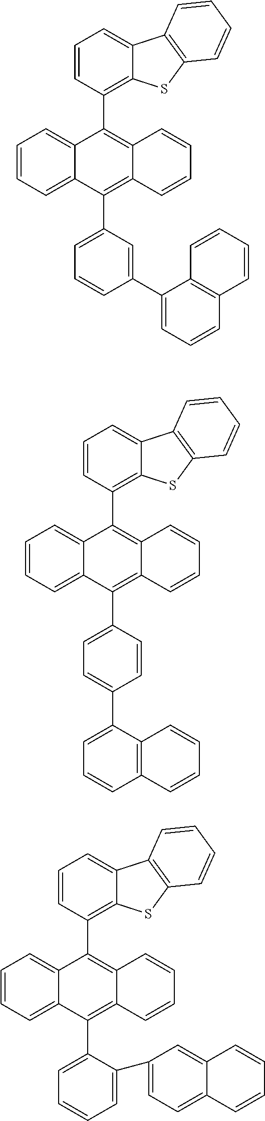

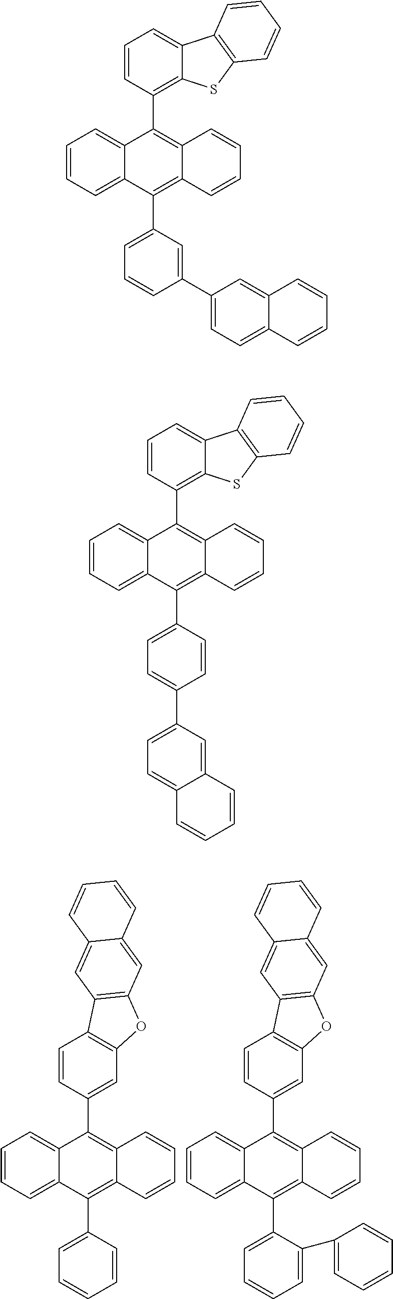

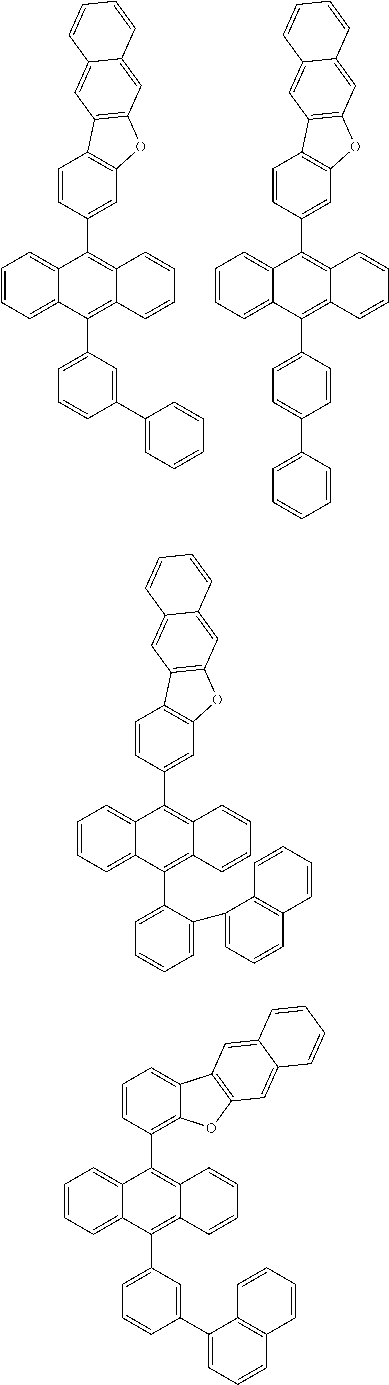

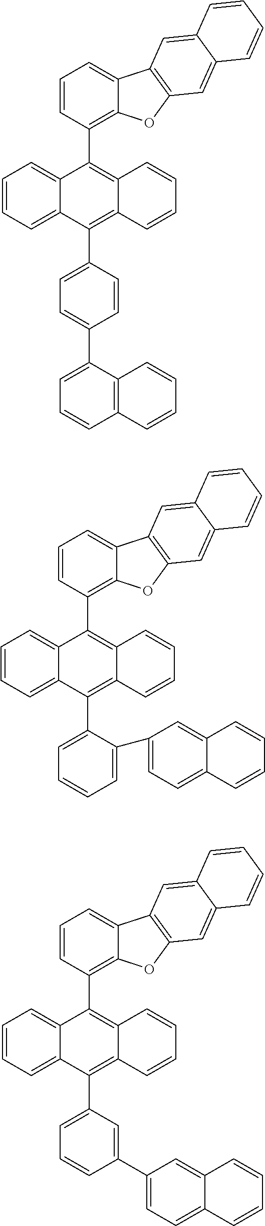 [SMILES: c1ccc2cc3c(cc2c1)oc1c(-c2c4ccccc4c(-c4ccc(-c5cccc6ccccc56)cc4)c4ccccc24)cccc13 ; c1ccc(-c2c3ccccc3c(-c3cccc4c3oc3cc5ccccc5cc34)c3ccccc23)c(-c2ccc3ccccc3c2)c1 ; c1cc(-c2ccc3ccccc3c2)cc(-c2c3ccccc3c(-c3cccc4c3oc3cc5ccccc5cc34)c3ccccc23)c1]

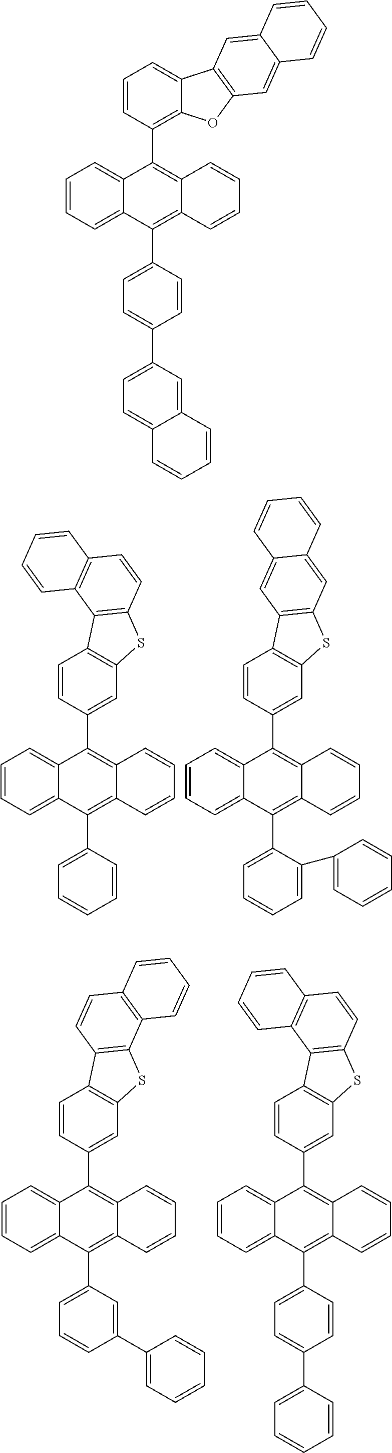

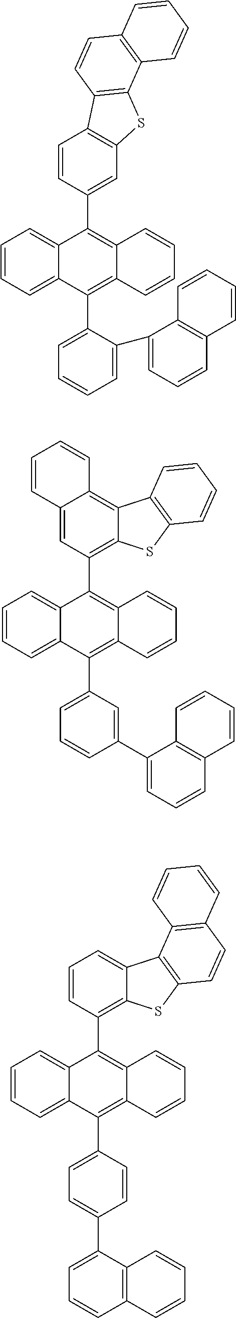 [SMILES: c1ccc(-c2c3ccccc3c(-c3ccc4c(c3)sc3c5ccccc5ccc43)c3ccccc23)c(-c2cccc3ccccc23)c1 ; c1cc(-c2cccc3ccccc23)cc(-c2c3ccccc3c(-c3cc4ccccc4c4c3sc3ccccc34)c3ccccc23)c1 ; c1ccc2c(-c3ccc(-c4c5ccccc5c(-c5cccc6c5sc5ccc7ccccc7c56)c5ccccc45)cc3)cccc2c1]

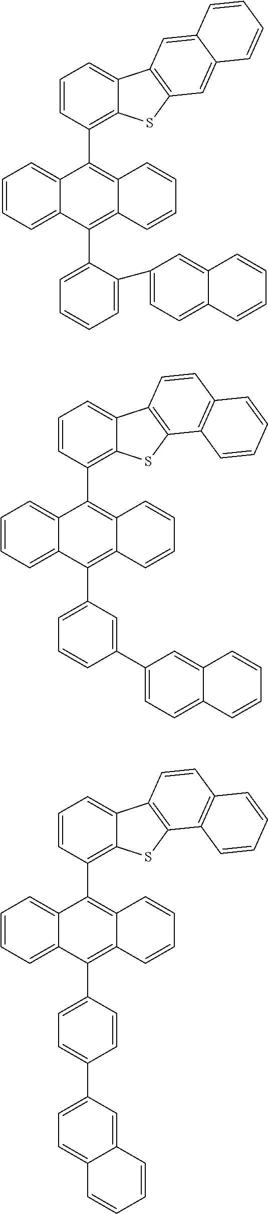 [SMILES: c1ccc(-c2c3ccccc3c(-c3cccc4c3sc3cc5ccccc5cc34)c3ccccc23)c(-c2ccc3ccccc3c2)c1 ; c1cc(-c2ccc3ccccc3c2)cc(-c2c3ccccc3c(-c3cccc4c3sc3c5ccccc5ccc43)c3ccccc23)c1 ; c1ccc2cc(-c3ccc(-c4c5ccccc5c(-c5cccc6c5sc5c7ccccc7ccc65)c5ccccc45)cc3)ccc2c1]

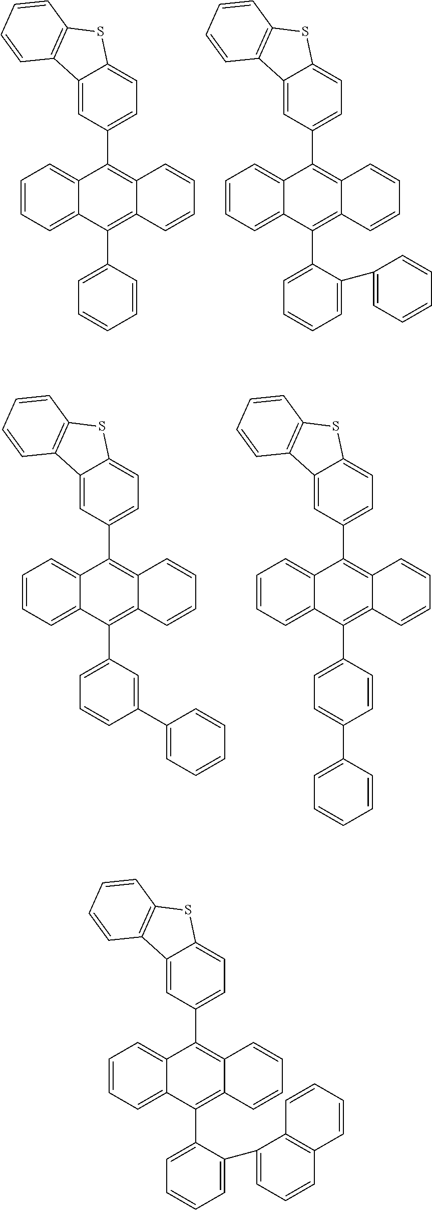

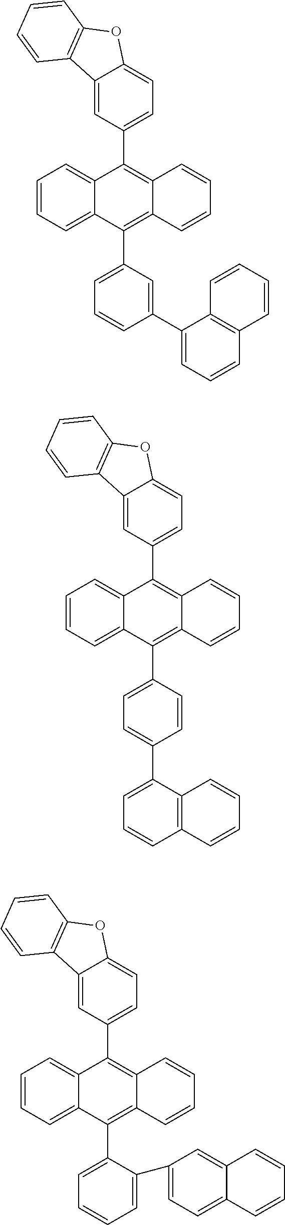 [SMILES: c1cc(-c2cccc3ccccc23)cc(-c2c3ccccc3c(-c3ccc4oc5ccccc5c4c3)c3ccccc23)c1 ; c1ccc2c(-c3ccc(-c4c5ccccc5c(-c5ccc6oc7ccccc7c6c5)c5ccccc45)cc3)cccc2c1 ; c1ccc(-c2c3ccccc3c(-c3ccc4oc5ccccc5c4c3)c3ccccc23)c(-c2ccc3ccccc3c2)c1]

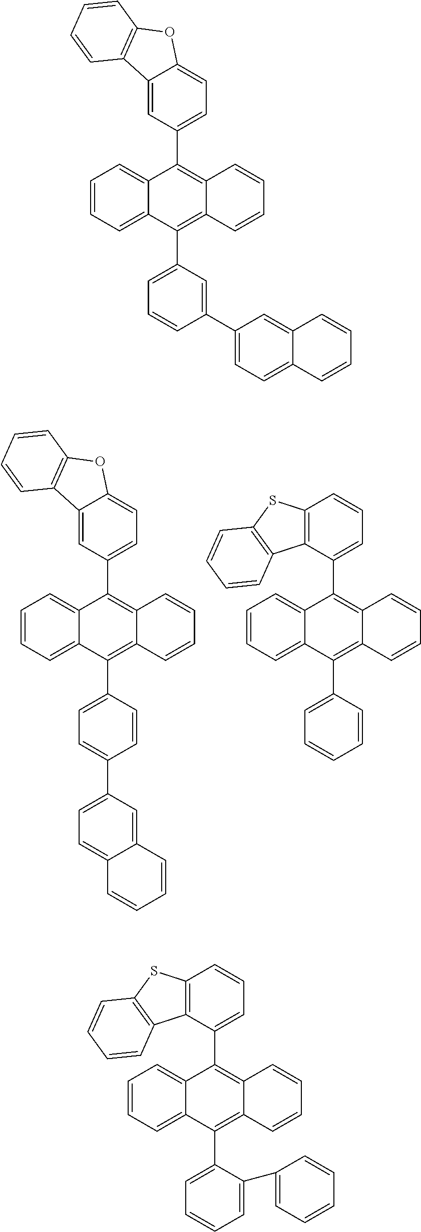

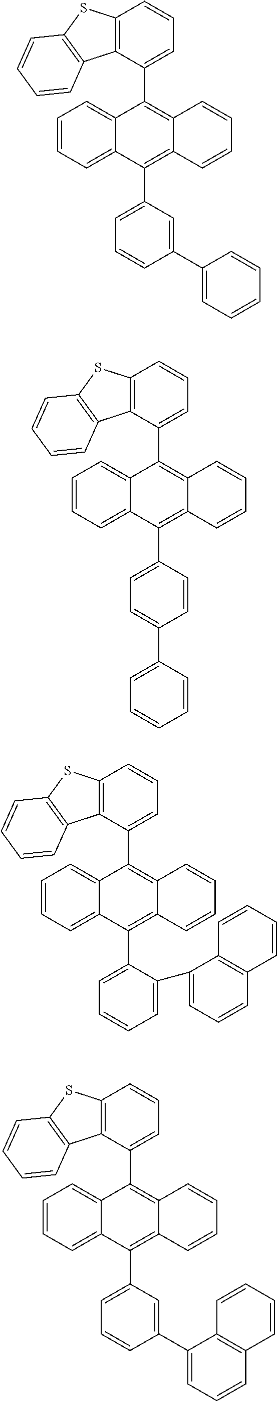 [SMILES: c1ccc(-c2cccc(-c3c4ccccc4c(-c4cccc5sc6ccccc6c45)c4ccccc34)c2)cc1 ; c1ccc(-c2ccc(-c3c4ccccc4c(-c4cccc5sc6ccccc6c45)c4ccccc34)cc2)cc1 ; c1ccc(-c2c3ccccc3c(-c3cccc4sc5ccccc5c34)c3ccccc23)c(-c2cccc3ccccc23)c1 ; c1cc(-c2cccc3ccccc23)cc(-c2c3ccccc3c(-c3cccc4sc5ccccc5c34)c3ccccc23)c1]

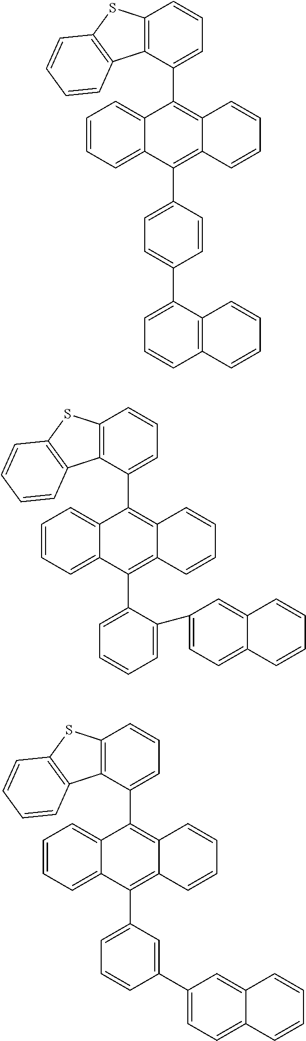 [SMILES: c1ccc2c(-c3ccc(-c4c5ccccc5c(-c5cccc6sc7ccccc7c56)c5ccccc45)cc3)cccc2c1 ; c1ccc(-c2c3ccccc3c(-c3cccc4sc5ccccc5c34)c3ccccc23)c(-c2ccc3ccccc3c2)c1 ; c1cc(-c2ccc3ccccc3c2)cc(-c2c3ccccc3c(-c3cccc4sc5ccccc5c34)c3ccccc23)c1]

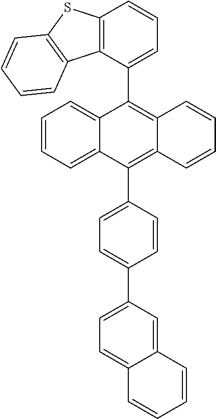 [SMILES: c1ccc2cc(-c3ccc(-c4c5ccccc5c(-c5cccc6sc7ccccc7c56)c5ccccc45)cc3)ccc2c1]

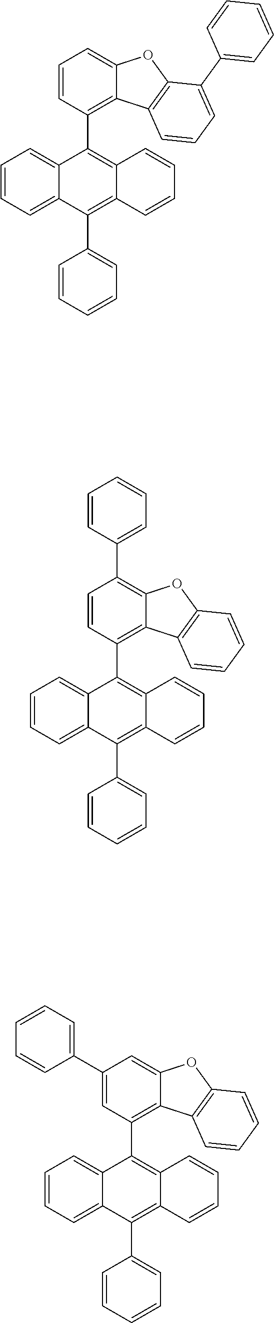 [SMILES: c1ccc(-c2c3ccccc3c(-c3cccc4oc5c(-c6ccccc6)cccc5c34)c3ccccc23)cc1 ; c1ccc(-c2c3ccccc3c(-c3ccc(-c4ccccc4)c4oc5ccccc5c34)c3ccccc23)cc1 ; c1ccc(-c2cc(-c3c4ccccc4c(-c4ccccc4)c4ccccc34)c3c(c2)oc2ccccc23)cc1]

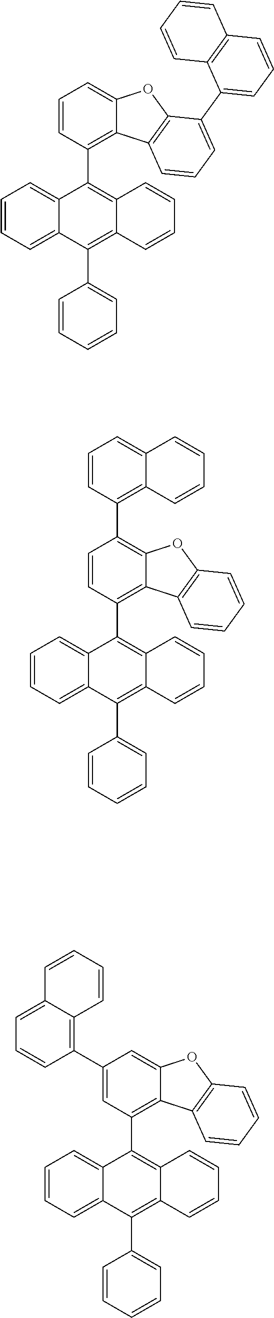 [SMILES: c1ccc(-c2c3ccccc3c(-c3cccc4oc5c(-c6cccc7ccccc67)cccc5c34)c3ccccc23)cc1 ; c1ccc(-c2c3ccccc3c(-c3ccc(-c4cccc5ccccc45)c4oc5ccccc5c34)c3ccccc23)cc1 ; c1ccc(-c2c3ccccc3c(-c3cc(-c4cccc5ccccc45)cc4oc5ccccc5c34)c3ccccc23)cc1]

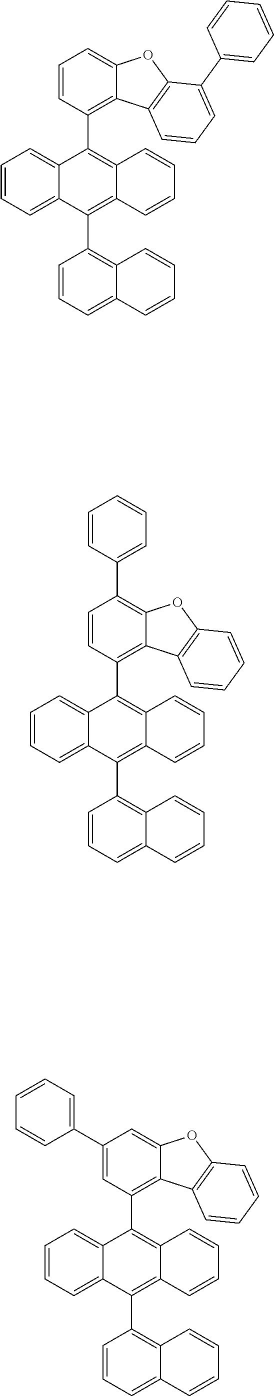 [SMILES: c1ccc(-c2cccc3c2oc2cccc(-c4c5ccccc5c(-c5cccc6ccccc56)c5ccccc45)c23)cc1 ; c1ccc(-c2ccc(-c3c4ccccc4c(-c4cccc5ccccc45)c4ccccc34)c3c2oc2ccccc23)cc1 ; c1ccc(-c2cc(-c3c4ccccc4c(-c4cccc5ccccc45)c4ccccc34)c3c(c2)oc2ccccc23)cc1]

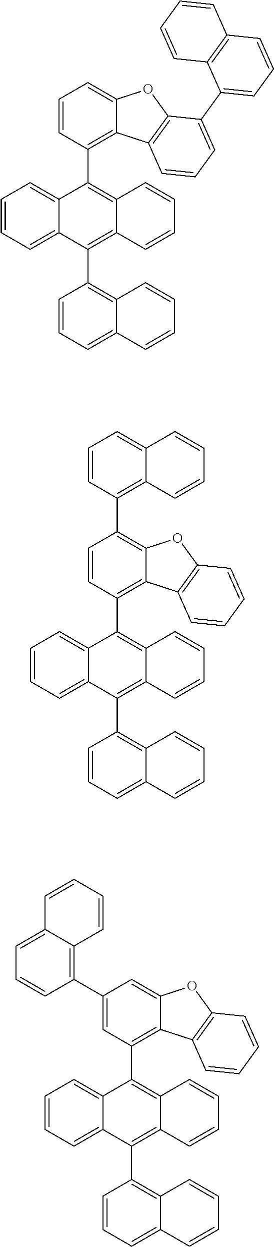 [SMILES: c1ccc2c(-c3c4ccccc4c(-c4cccc5oc6c(-c7cccc8ccccc78)cccc6c45)c4ccccc34)cccc2c1 ; c1ccc2c(-c3c4ccccc4c(-c4ccc(-c5cccc6ccccc56)c5oc6ccccc6c45)c4ccccc34)cccc2c1 ; c1ccc2c(-c3cc(-c4c5ccccc5c(-c5cccc6ccccc56)c5ccccc45)c4c(c3)oc3ccccc34)cccc2c1]

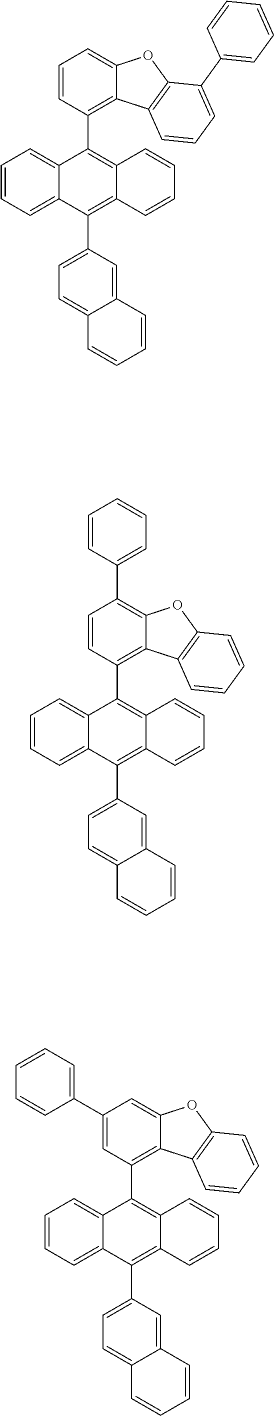 [SMILES: c1ccc(-c2cccc3c2oc2cccc(-c4c5ccccc5c(-c5ccc6ccccc6c5)c5ccccc45)c23)cc1 ; c1ccc(-c2ccc(-c3c4ccccc4c(-c4ccc5ccccc5c4)c4ccccc34)c3c2oc2ccccc23)cc1 ; c1ccc(-c2cc(-c3c4ccccc4c(-c4ccc5ccccc5c4)c4ccccc34)c3c(c2)oc2ccccc23)cc1]

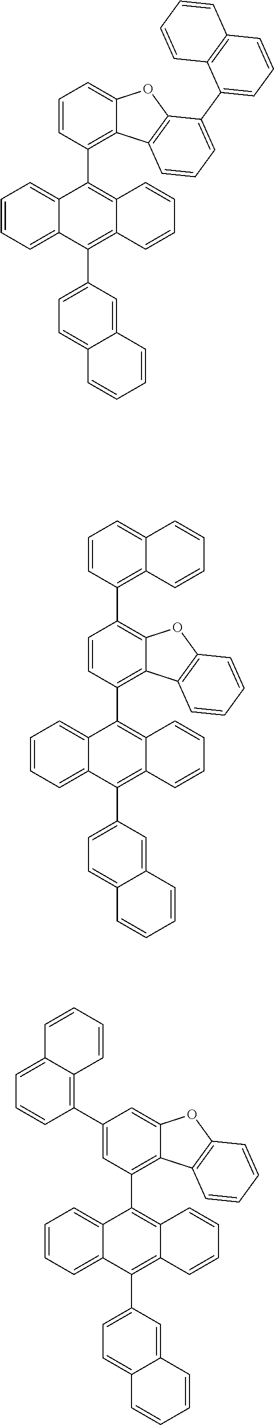 [SMILES: c1ccc2cc(-c3c4ccccc4c(-c4cccc5oc6c(-c7cccc8ccccc78)cccc6c45)c4ccccc34)ccc2c1 ; c1ccc2cc(-c3c4ccccc4c(-c4ccc(-c5cccc6ccccc56)c5oc6ccccc6c45)c4ccccc34)ccc2c1 ; c1ccc2cc(-c3c4ccccc4c(-c4cc(-c5cccc6ccccc56)cc5oc6ccccc6c45)c4ccccc34)ccc2c1]

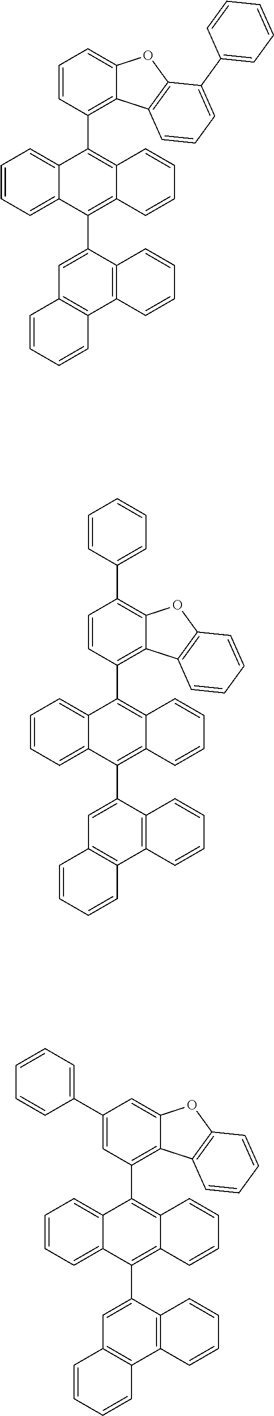 [SMILES: c1ccc(-c2cccc3c2oc2cccc(-c4c5ccccc5c(-c5cc6ccccc6c6ccccc56)c5ccccc45)c23)cc1 ; c1ccc(-c2ccc(-c3c4ccccc4c(-c4cc5ccccc5c5ccccc45)c4ccccc34)c3c2oc2ccccc23)cc1 ; c1ccc(-c2cc(-c3c4ccccc4c(-c4cc5ccccc5c5ccccc45)c4ccccc34)c3c(c2)oc2ccccc23)cc1]

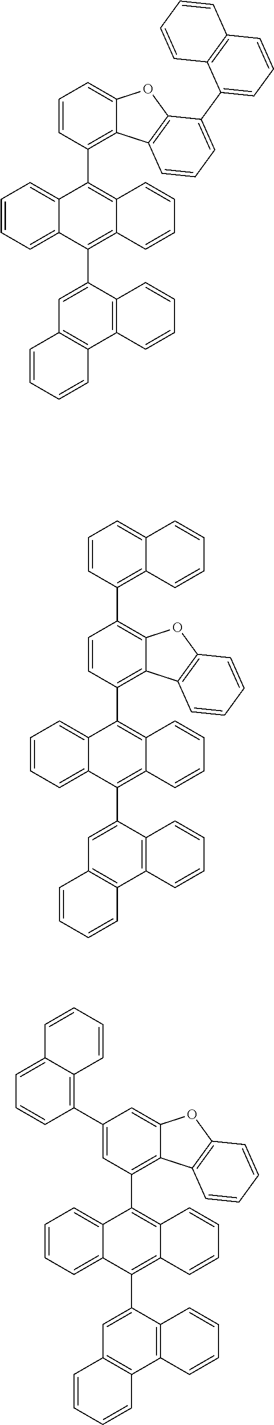 [SMILES: c1ccc2c(-c3cccc4c3oc3cccc(-c5c6ccccc6c(-c6cc7ccccc7c7ccccc67)c6ccccc56)c34)cccc2c1 ; c1ccc2c(-c3ccc(-c4c5ccccc5c(-c5cc6ccccc6c6ccccc56)c5ccccc45)c4c3oc3ccccc34)cccc2c1 ; c1ccc2c(-c3cc(-c4c5ccccc5c(-c5cc6ccccc6c6ccccc56)c5ccccc45)c4c(c3)oc3ccccc34)cccc2c1]

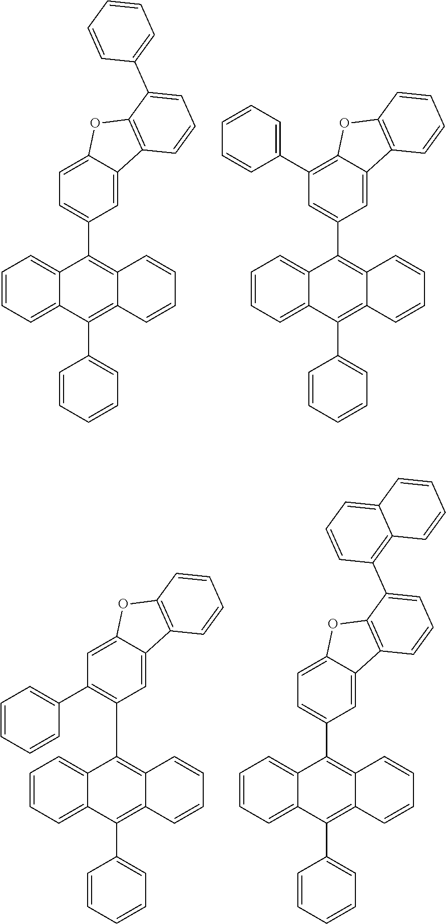

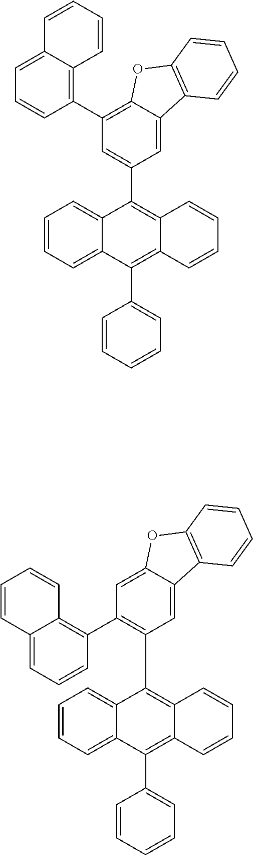 [SMILES: c1ccc(-c2c3ccccc3c(-c3cc(-c4cccc5ccccc45)c4oc5ccccc5c4c3)c3ccccc23)cc1 ; c1ccc(-c2c3ccccc3c(-c3cc4c(cc3-c3cccc5ccccc35)oc3ccccc34)c3ccccc23)cc1]

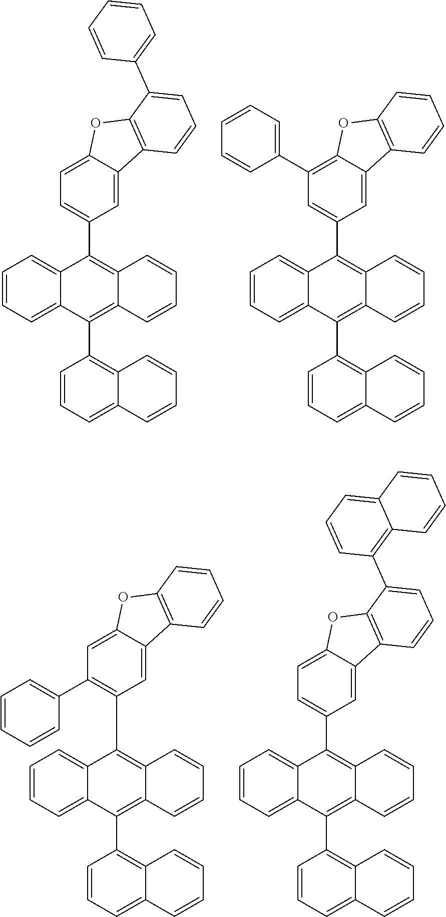

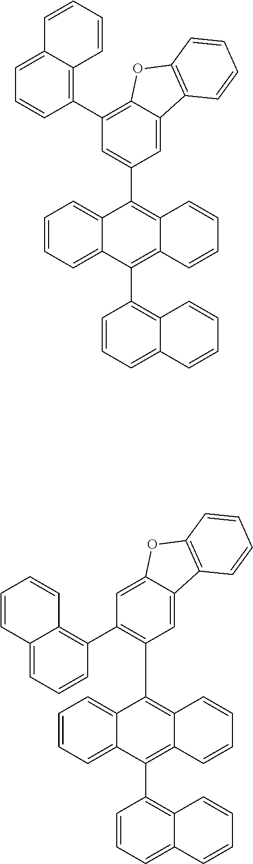 [SMILES: c1ccc2c(-c3c4ccccc4c(-c4cc(-c5cccc6ccccc56)c5oc6ccccc6c5c4)c4ccccc34)cccc2c1 ; c1ccc2c(-c3cc4oc5ccccc5c4cc3-c3c4ccccc4c(-c4cccc5ccccc45)c4ccccc34)cccc2c1]

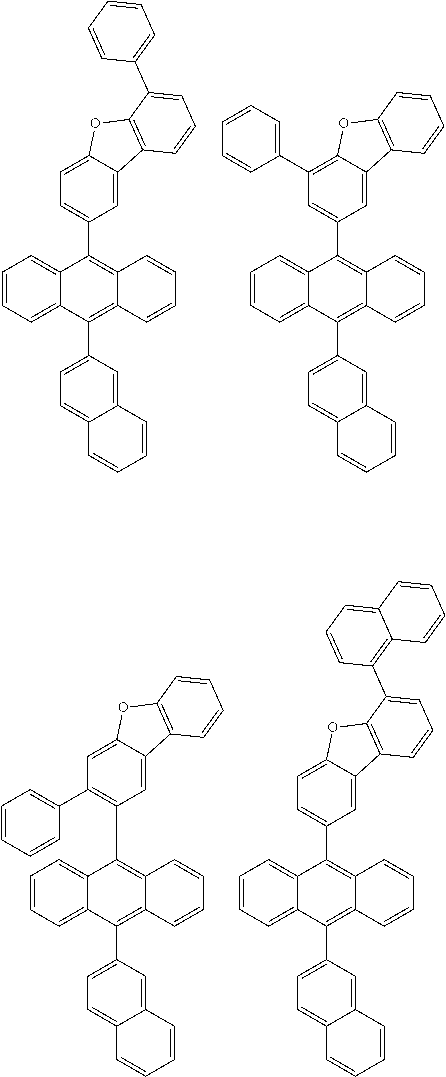

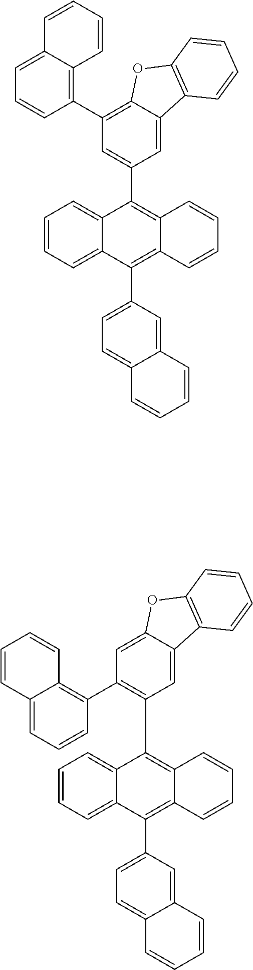 [SMILES: c1ccc2cc(-c3c4ccccc4c(-c4cc(-c5cccc6ccccc56)c5oc6ccccc6c5c4)c4ccccc34)ccc2c1 ; c1ccc2cc(-c3c4ccccc4c(-c4cc5c(cc4-c4cccc6ccccc46)oc4ccccc45)c4ccccc34)ccc2c1]

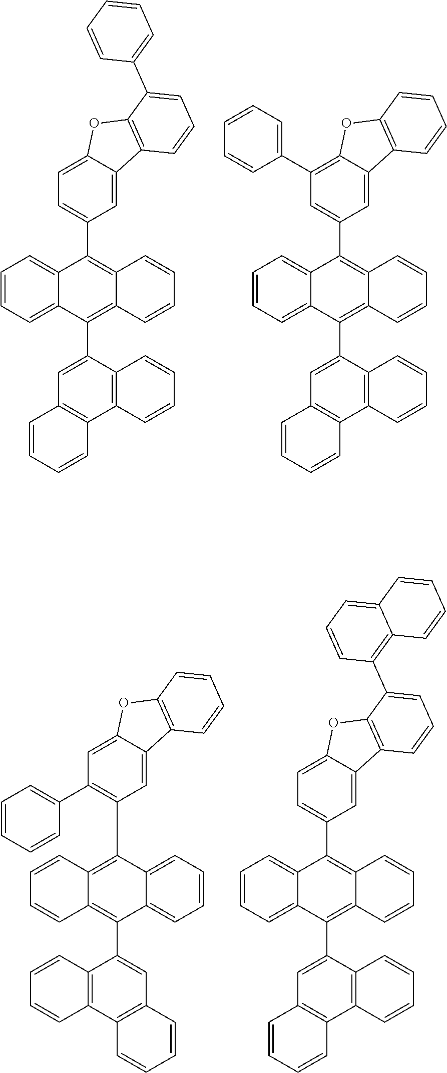

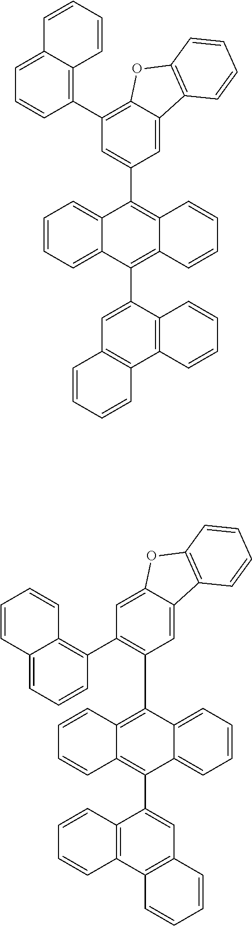 [SMILES: c1ccc2c(-c3cc(-c4c5ccccc5c(-c5cc6ccccc6c6ccccc56)c5ccccc45)cc4c3oc3ccccc34)cccc2c1 ; c1ccc2c(-c3cc4oc5ccccc5c4cc3-c3c4ccccc4c(-c4cc5ccccc5c5ccccc45)c4ccccc34)cccc2c1]

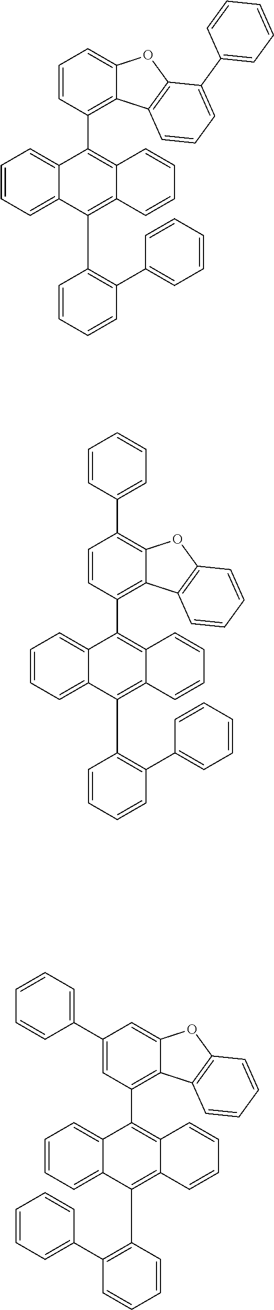 [SMILES: c1ccc(-c2ccccc2-c2c3ccccc3c(-c3cccc4oc5c(-c6ccccc6)cccc5c34)c3ccccc23)cc1 ; c1ccc(-c2ccccc2-c2c3ccccc3c(-c3ccc(-c4ccccc4)c4oc5ccccc5c34)c3ccccc23)cc1 ; c1ccc(-c2cc(-c3c4ccccc4c(-c4ccccc4-c4ccccc4)c4ccccc34)c3c(c2)oc2ccccc23)cc1]

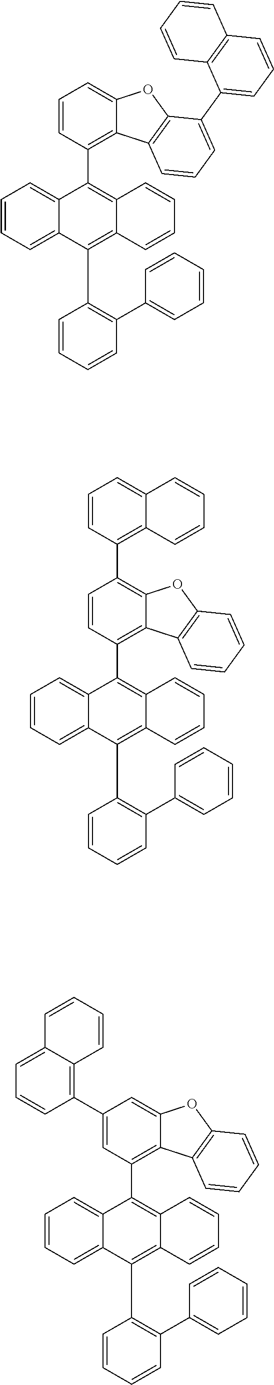 [SMILES: c1ccc(-c2ccccc2-c2c3ccccc3c(-c3cccc4oc5c(-c6cccc7ccccc67)cccc5c34)c3ccccc23)cc1 ; c1ccc(-c2ccccc2-c2c3ccccc3c(-c3ccc(-c4cccc5ccccc45)c4oc5ccccc5c34)c3ccccc23)cc1 ; c1ccc(-c2ccccc2-c2c3ccccc3c(-c3cc(-c4cccc5ccccc45)cc4oc5ccccc5c34)c3ccccc23)cc1]

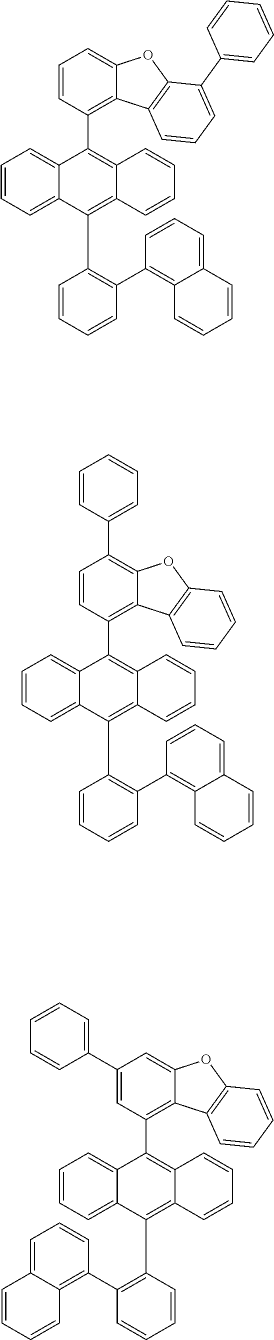 [SMILES: c1ccc(-c2cccc3c2oc2cccc(-c4c5ccccc5c(-c5ccccc5-c5cccc6ccccc56)c5ccccc45)c23)cc1 ; c1ccc(-c2ccc(-c3c4ccccc4c(-c4ccccc4-c4cccc5ccccc45)c4ccccc34)c3c2oc2ccccc23)cc1 ; c1ccc(-c2cc(-c3c4ccccc4c(-c4ccccc4-c4cccc5ccccc45)c4ccccc34)c3c(c2)oc2ccccc23)cc1]

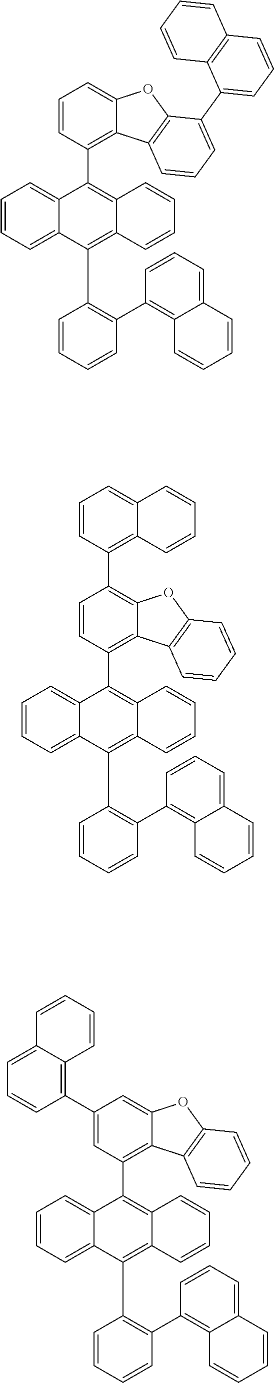 [SMILES: c1ccc(-c2c3ccccc3c(-c3cccc4oc5c(-c6cccc7ccccc67)cccc5c34)c3ccccc23)c(-c2cccc3ccccc23)c1 ; c1ccc(-c2c3ccccc3c(-c3ccc(-c4cccc5ccccc45)c4oc5ccccc5c34)c3ccccc23)c(-c2cccc3ccccc23)c1 ; c1ccc(-c2c3ccccc3c(-c3cc(-c4cccc5ccccc45)cc4oc5ccccc5c34)c3ccccc23)c(-c2cccc3ccccc23)c1]

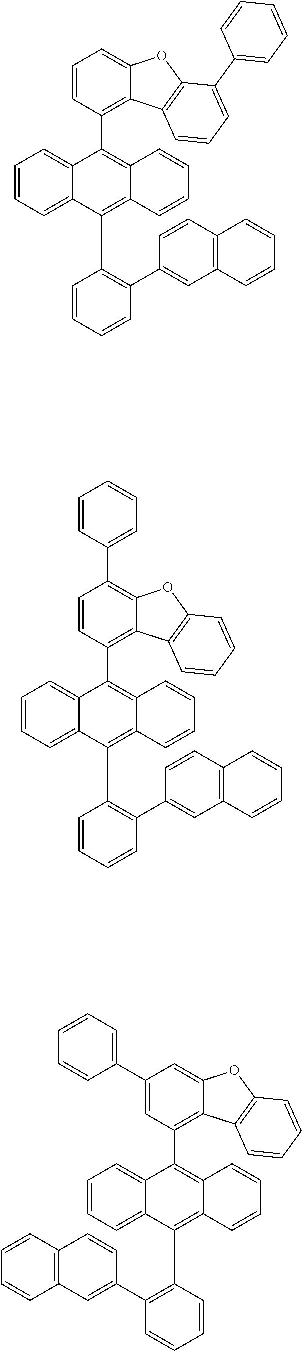 [SMILES: c1ccc(-c2cccc3c2oc2cccc(-c4c5ccccc5c(-c5ccccc5-c5ccc6ccccc6c5)c5ccccc45)c23)cc1 ; c1ccc(-c2ccc(-c3c4ccccc4c(-c4ccccc4-c4ccc5ccccc5c4)c4ccccc34)c3c2oc2ccccc23)cc1 ; c1ccc(-c2cc(-c3c4ccccc4c(-c4ccccc4-c4ccc5ccccc5c4)c4ccccc34)c3c(c2)oc2ccccc23)cc1]

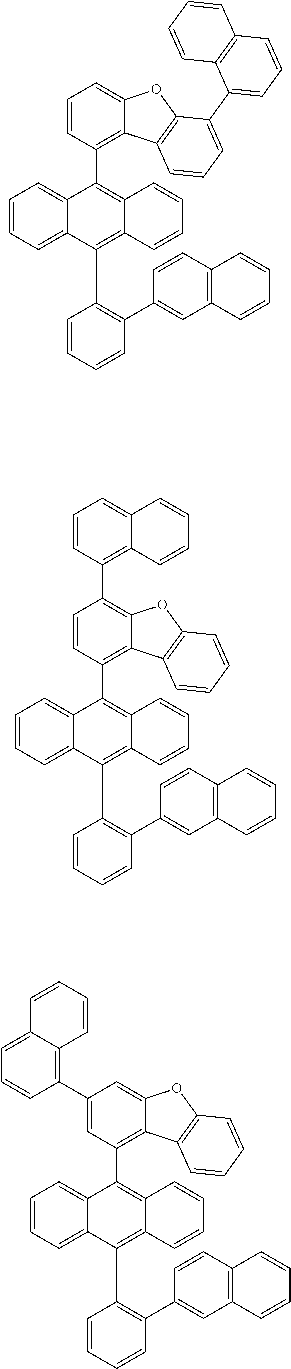 [SMILES: c1ccc(-c2c3ccccc3c(-c3cccc4oc5c(-c6cccc7ccccc67)cccc5c34)c3ccccc23)c(-c2ccc3ccccc3c2)c1 ; c1ccc(-c2c3ccccc3c(-c3ccc(-c4cccc5ccccc45)c4oc5ccccc5c34)c3ccccc23)c(-c2ccc3ccccc3c2)c1 ; c1ccc(-c2c3ccccc3c(-c3cc(-c4cccc5ccccc45)cc4oc5ccccc5c34)c3ccccc23)c(-c2ccc3ccccc3c2)c1]

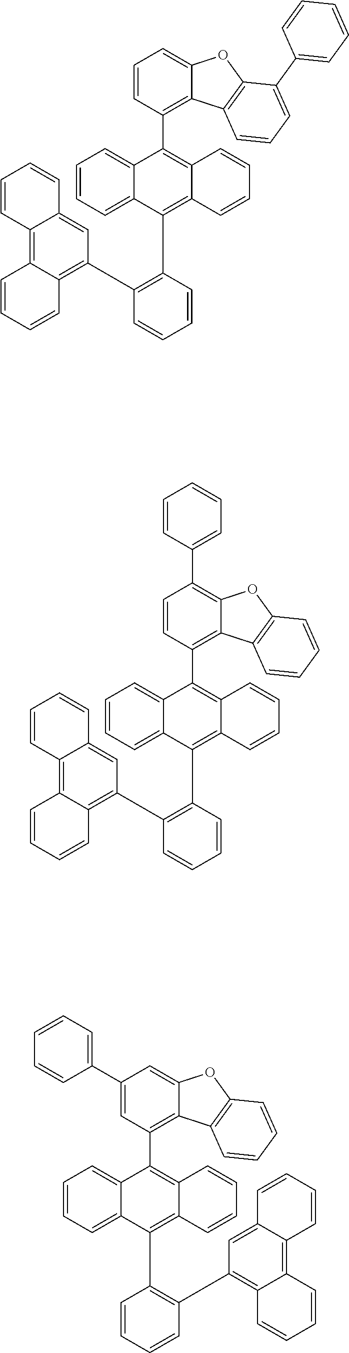 [SMILES: c1ccc(-c2cccc3c2oc2cccc(-c4c5ccccc5c(-c5ccccc5-c5cc6ccccc6c6ccccc56)c5ccccc45)c23)cc1 ; c1ccc(-c2ccc(-c3c4ccccc4c(-c4ccccc4-c4cc5ccccc5c5ccccc45)c4ccccc34)c3c2oc2ccccc23)cc1 ; c1ccc(-c2cc(-c3c4ccccc4c(-c4ccccc4-c4cc5ccccc5c5ccccc45)c4ccccc34)c3c(c2)oc2ccccc23)cc1]

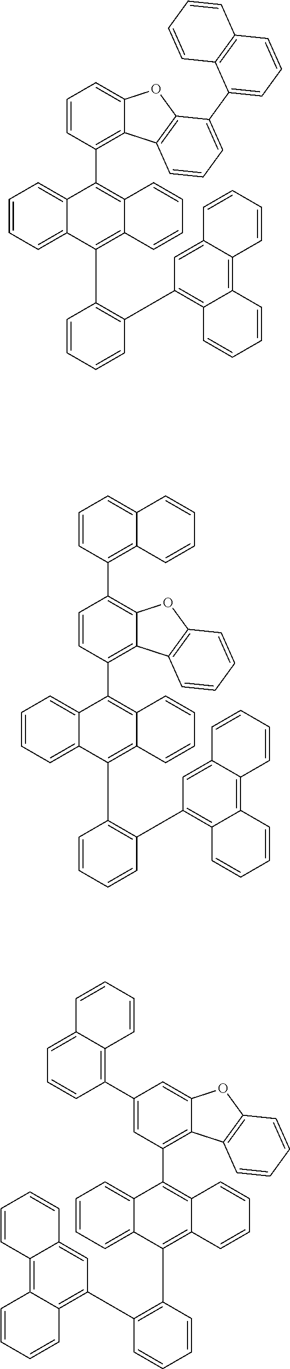 [SMILES: c1ccc(-c2cc3ccccc3c3ccccc23)c(-c2c3ccccc3c(-c3cccc4oc5c(-c6cccc7ccccc67)cccc5c34)c3ccccc23)c1 ; c1ccc(-c2cc3ccccc3c3ccccc23)c(-c2c3ccccc3c(-c3ccc(-c4cccc5ccccc45)c4oc5ccccc5c34)c3ccccc23)c1 ; c1ccc(-c2cc3ccccc3c3ccccc23)c(-c2c3ccccc3c(-c3cc(-c4cccc5ccccc45)cc4oc5ccccc5c34)c3ccccc23)c1]

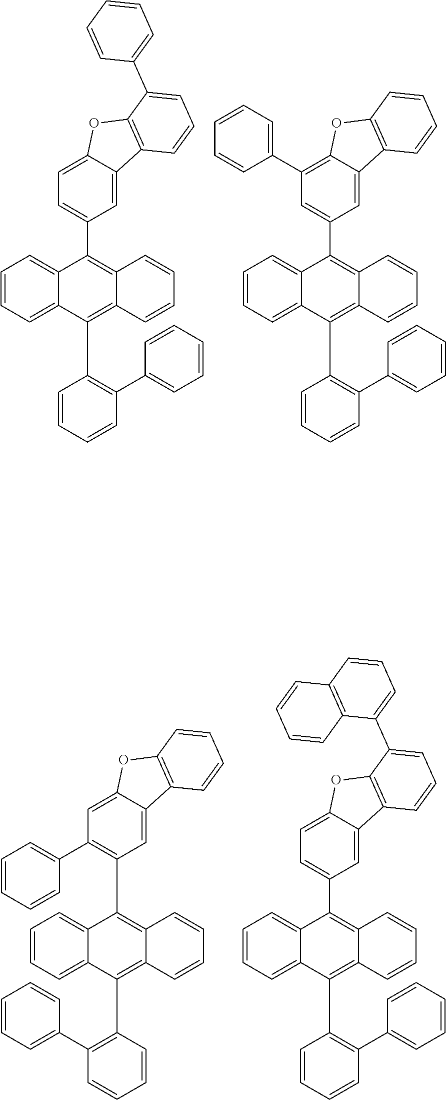

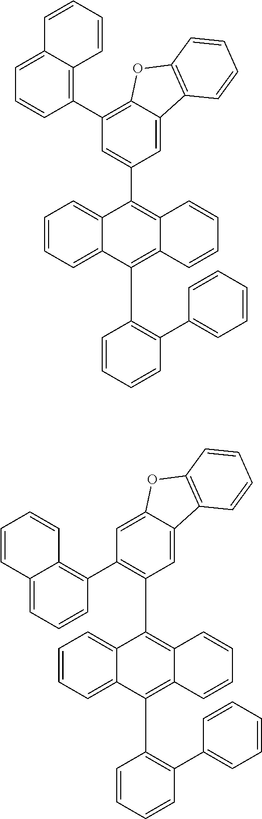 [SMILES: c1ccc(-c2ccccc2-c2c3ccccc3c(-c3cc(-c4cccc5ccccc45)c4oc5ccccc5c4c3)c3ccccc23)cc1 ; c1ccc(-c2ccccc2-c2c3ccccc3c(-c3cc4c(cc3-c3cccc5ccccc35)oc3ccccc34)c3ccccc23)cc1]

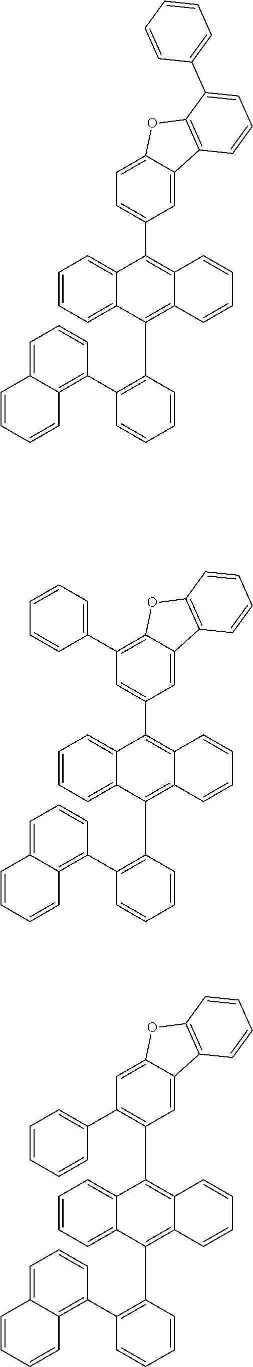 [SMILES: c1ccc(-c2cccc3c2oc2ccc(-c4c5ccccc5c(-c5ccccc5-c5cccc6ccccc56)c5ccccc45)cc23)cc1 ; c1ccc(-c2cc(-c3c4ccccc4c(-c4ccccc4-c4cccc5ccccc45)c4ccccc34)cc3c2oc2ccccc23)cc1 ; c1ccc(-c2cc3oc4ccccc4c3cc2-c2c3ccccc3c(-c3ccccc3-c3cccc4ccccc34)c3ccccc23)cc1]

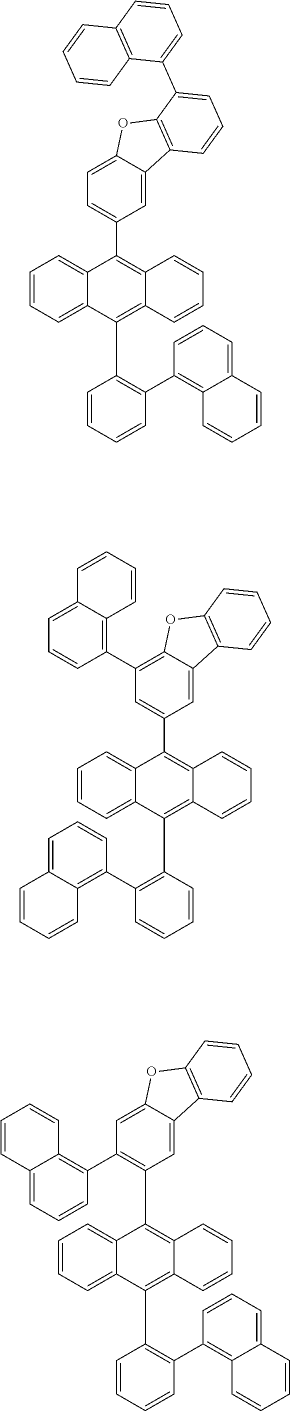 [SMILES: c1ccc(-c2c3ccccc3c(-c3ccc4oc5c(-c6cccc7ccccc67)cccc5c4c3)c3ccccc23)c(-c2cccc3ccccc23)c1 ; c1ccc(-c2c3ccccc3c(-c3cc(-c4cccc5ccccc45)c4oc5ccccc5c4c3)c3ccccc23)c(-c2cccc3ccccc23)c1 ; c1ccc(-c2c3ccccc3c(-c3cc4c(cc3-c3cccc5ccccc35)oc3ccccc34)c3ccccc23)c(-c2cccc3ccccc23)c1]

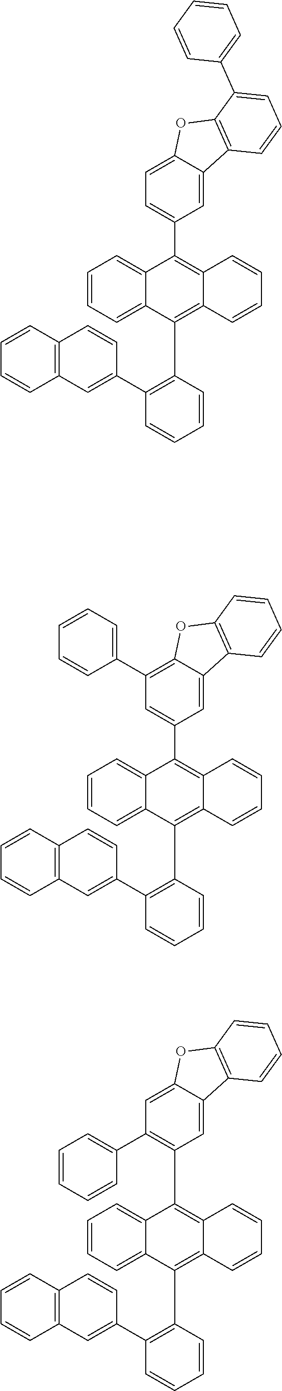 [SMILES: c1ccc(-c2cccc3c2oc2ccc(-c4c5ccccc5c(-c5ccccc5-c5ccc6ccccc6c5)c5ccccc45)cc23)cc1 ; c1ccc(-c2cc(-c3c4ccccc4c(-c4ccccc4-c4ccc5ccccc5c4)c4ccccc34)cc3c2oc2ccccc23)cc1 ; c1ccc(-c2cc3oc4ccccc4c3cc2-c2c3ccccc3c(-c3ccccc3-c3ccc4ccccc4c3)c3ccccc23)cc1]

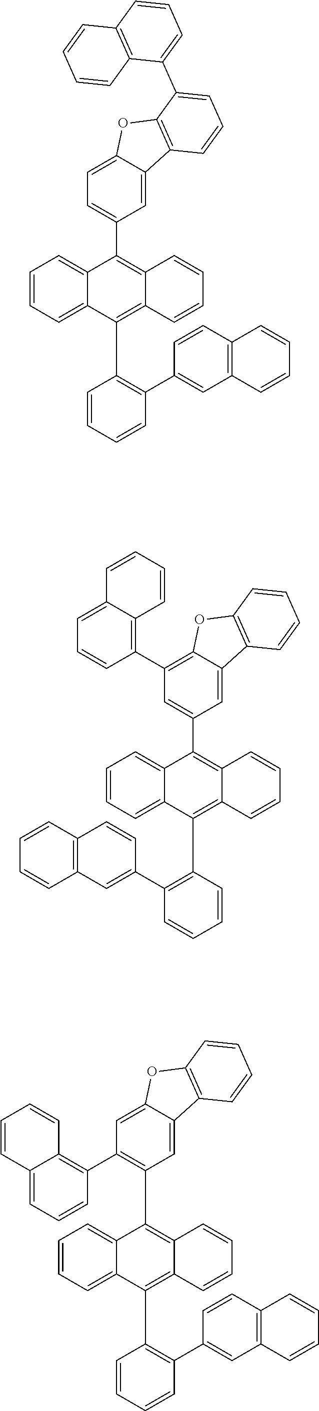 [SMILES: c1ccc(-c2c3ccccc3c(-c3ccc4oc5c(-c6cccc7ccccc67)cccc5c4c3)c3ccccc23)c(-c2ccc3ccccc3c2)c1 ; c1ccc(-c2c3ccccc3c(-c3cc(-c4cccc5ccccc45)c4oc5ccccc5c4c3)c3ccccc23)c(-c2ccc3ccccc3c2)c1 ; c1ccc(-c2c3ccccc3c(-c3cc4c(cc3-c3cccc5ccccc35)oc3ccccc34)c3ccccc23)c(-c2ccc3ccccc3c2)c1]

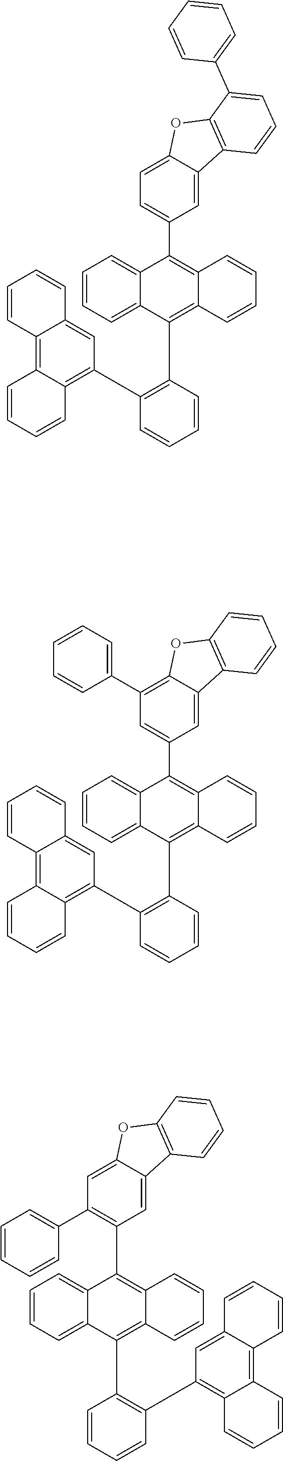 [SMILES: c1ccc(-c2cccc3c2oc2ccc(-c4c5ccccc5c(-c5ccccc5-c5cc6ccccc6c6ccccc56)c5ccccc45)cc23)cc1 ; c1ccc(-c2cc(-c3c4ccccc4c(-c4ccccc4-c4cc5ccccc5c5ccccc45)c4ccccc34)cc3c2oc2ccccc23)cc1 ; c1ccc(-c2cc3oc4ccccc4c3cc2-c2c3ccccc3c(-c3ccccc3-c3cc4ccccc4c4ccccc34)c3ccccc23)cc1]

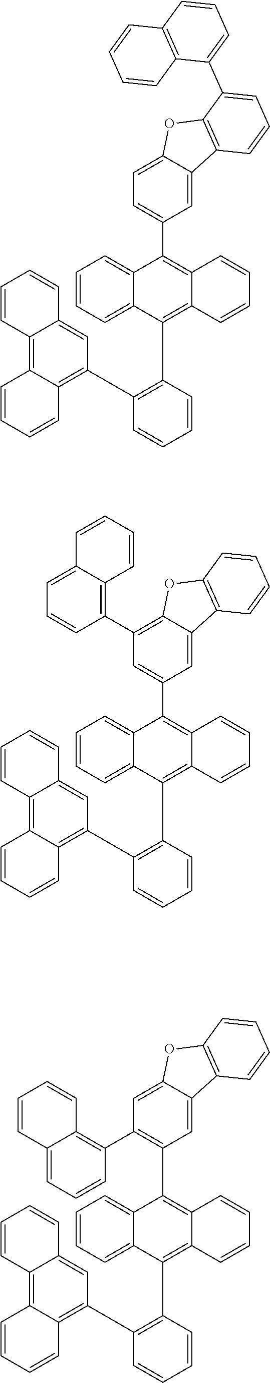 [SMILES: c1ccc(-c2cc3ccccc3c3ccccc23)c(-c2c3ccccc3c(-c3ccc4oc5c(-c6cccc7ccccc67)cccc5c4c3)c3ccccc23)c1 ; c1ccc(-c2cc3ccccc3c3ccccc23)c(-c2c3ccccc3c(-c3cc(-c4cccc5ccccc45)c4oc5ccccc5c4c3)c3ccccc23)c1 ; c1ccc(-c2cc3ccccc3c3ccccc23)c(-c2c3ccccc3c(-c3cc4c(cc3-c3cccc5ccccc35)oc3ccccc34)c3ccccc23)c1]

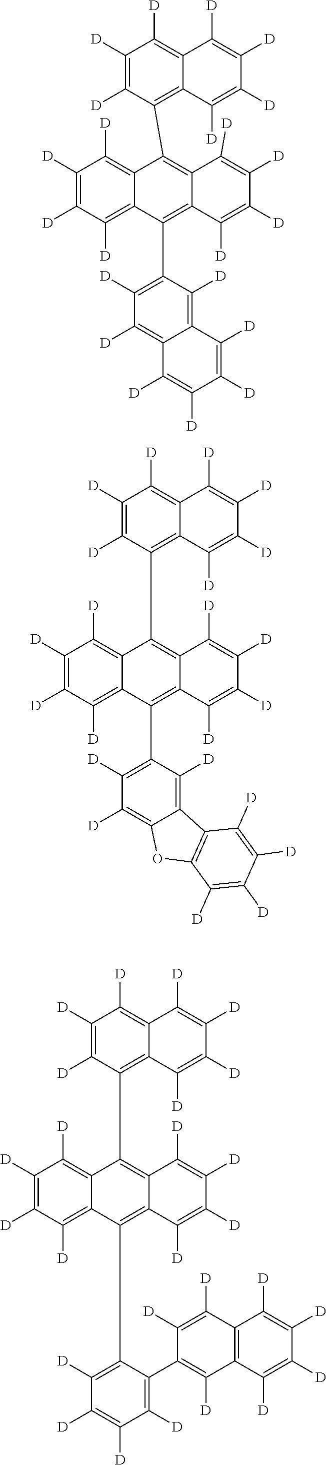

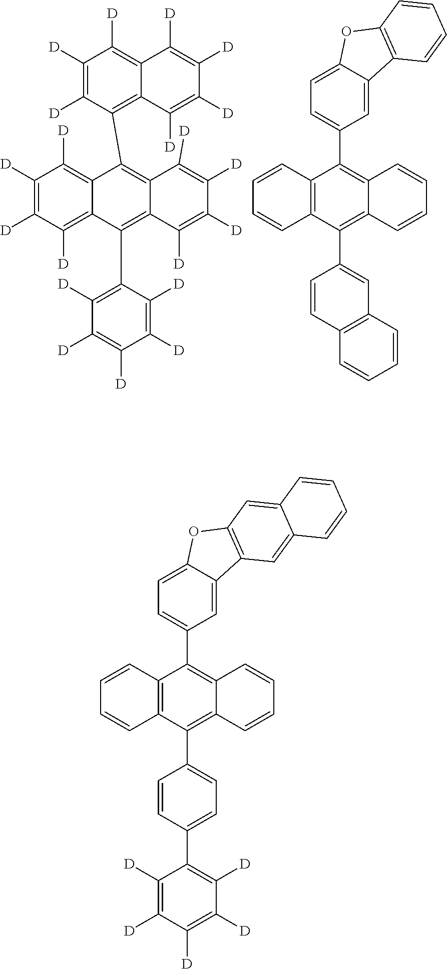

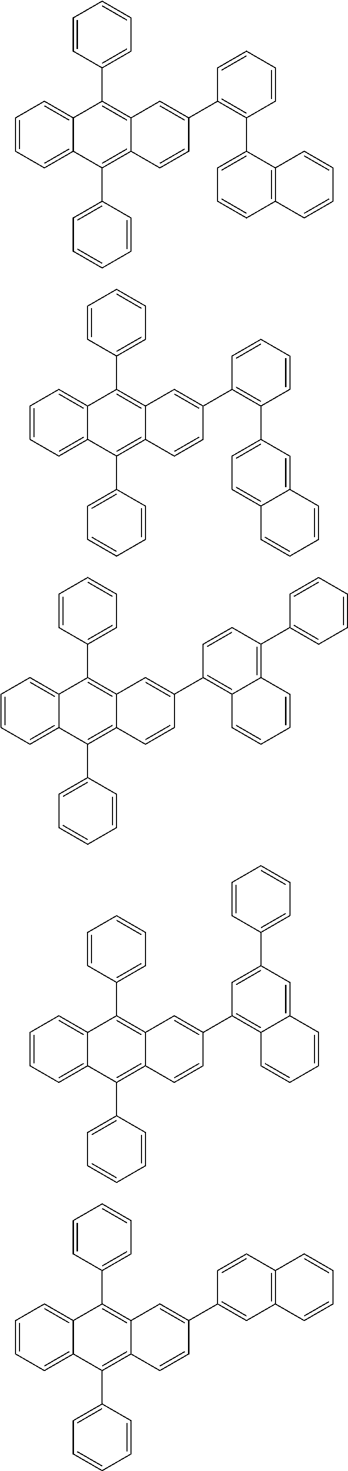

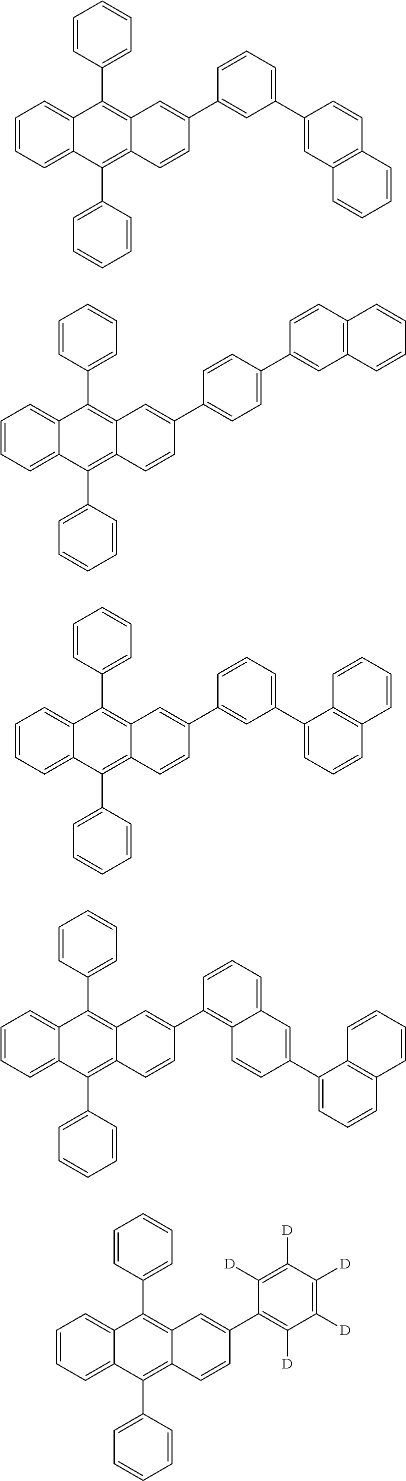

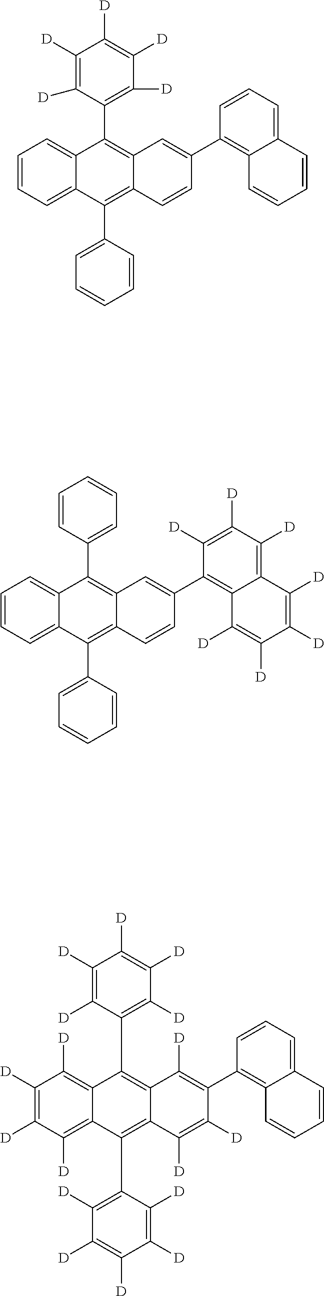 [SMILES: [2H]c1c([2H])c([2H])c(-c2c3ccccc3c(-c3ccccc3)c3ccc(-c4cccc5ccccc45)cc23)c([2H])c1[2H] ; [2H]c1c([2H])c([2H])c2c(-c3ccc4c(-c5ccccc5)c5ccccc5c(-c5ccccc5)c4c3)c([2H])c([2H])c([2H])c2c1[2H] ; [2H]c1c([2H])c([2H])c(-c2c3c([2H])c([2H])c([2H])c([2H])c3c(-c3c([2H])c([2H])c([2H])c([2H])c3[2H])c3c([2H])c(-c4cccc5ccccc45)c([2H])c([2H])c23)c([2H])c1[2H]]

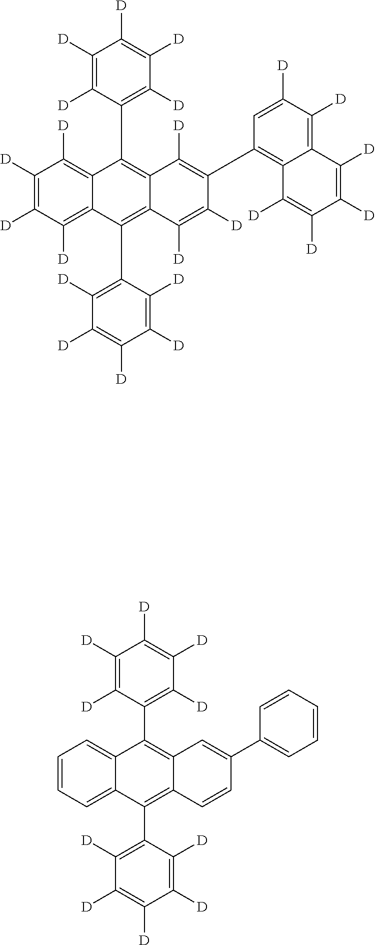 [SMILES: [2H]c1cc(-c2c([2H])c([2H])c3c(-c4c([2H])c([2H])c([2H])c([2H])c4[2H])c4c([2H])c([2H])c([2H])c([2H])c4c(-c4c([2H])c([2H])c([2H])c([2H])c4[2H])c3c2[2H])c2c([2H])c([2H])c([2H])c([2H])c2c1[2H] ; [2H]c1c([2H])c([2H])c(-c2c3ccccc3c(-c3c([2H])c([2H])c([2H])c([2H])c3[2H])c3cc(-c4ccccc4)ccc23)c([2H])c1[2H]]

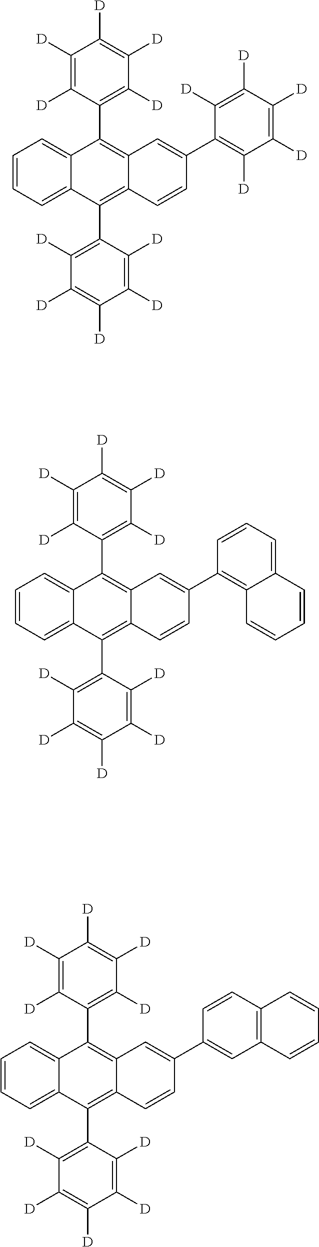 [SMILES: [2H]c1c([2H])c([2H])c(-c2ccc3c(-c4c([2H])c([2H])c([2H])c([2H])c4[2H])c4ccccc4c(-c4c([2H])c([2H])c([2H])c([2H])c4[2H])c3c2)c([2H])c1[2H] ; [2H]c1c([2H])c([2H])c(-c2c3ccccc3c(-c3c([2H])c([2H])c([2H])c([2H])c3[2H])c3cc(-c4cccc5ccccc45)ccc23)c([2H])c1[2H] ; [2H]c1c([2H])c([2H])c(-c2c3ccccc3c(-c3c([2H])c([2H])c([2H])c([2H])c3[2H])c3cc(-c4ccc5ccccc5c4)ccc23)c([2H])c1[2H]]

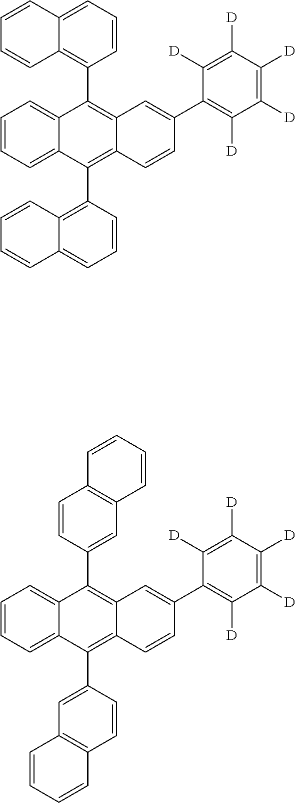 [SMILES: [2H]c1c([2H])c([2H])c(-c2ccc3c(-c4cccc5ccccc45)c4ccccc4c(-c4cccc5ccccc45)c3c2)c([2H])c1[2H] ; [2H]c1c([2H])c([2H])c(-c2ccc3c(-c4ccc5ccccc5c4)c4ccccc4c(-c4ccc5ccccc5c4)c3c2)c([2H])c1[2H]]

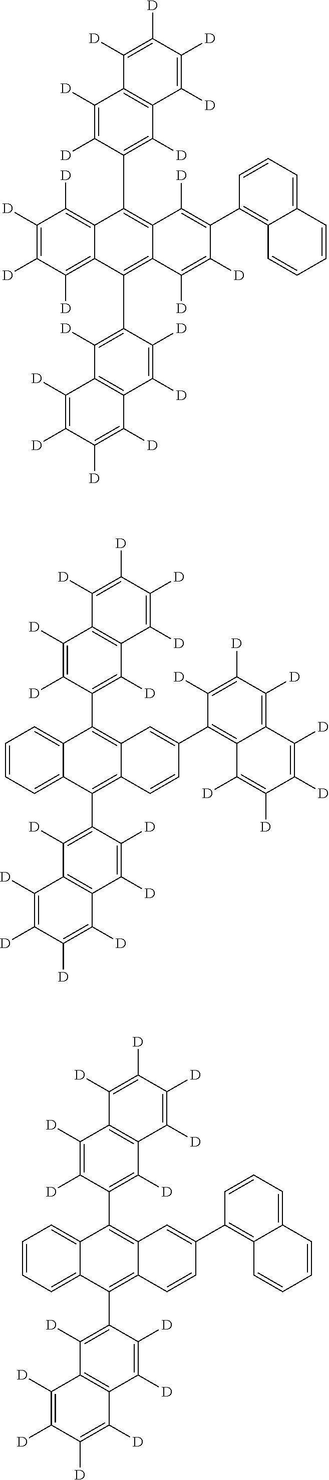

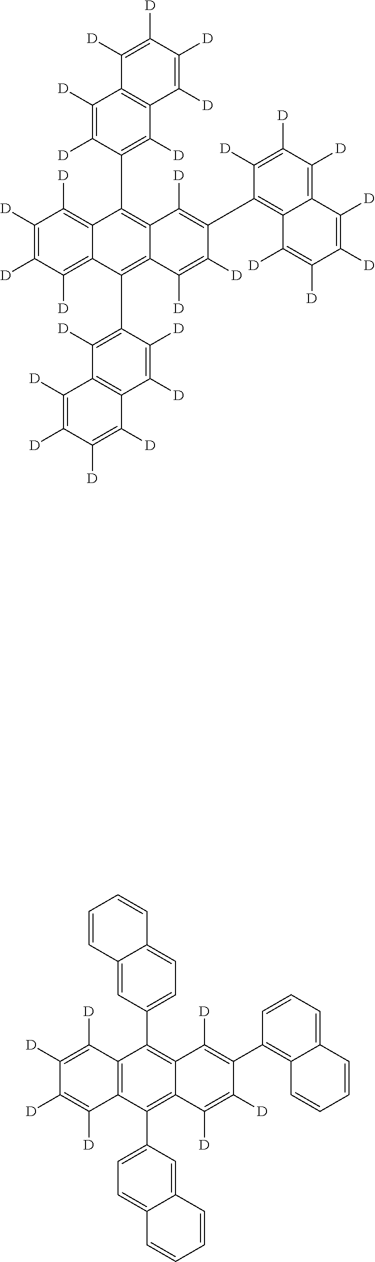 [SMILES: [2H]c1c([2H])c([2H])c2c([2H])c(-c3c4c([2H])c([2H])c([2H])c([2H])c4c(-c4c([2H])c([2H])c5c([2H])c([2H])c([2H])c([2H])c5c4[2H])c4c([2H])c(-c5c([2H])c([2H])c([2H])c6c([2H])c([2H])c([2H])c([2H])c56)c([2H])c([2H])c34)c([2H])c([2H])c2c1[2H] ; [2H]c1c([2H])c([2H])c2c(-c3ccc4ccccc4c3)c3c([2H])c(-c4cccc5ccccc45)c([2H])c([2H])c3c(-c3ccc4ccccc4c3)c2c1[2H]]

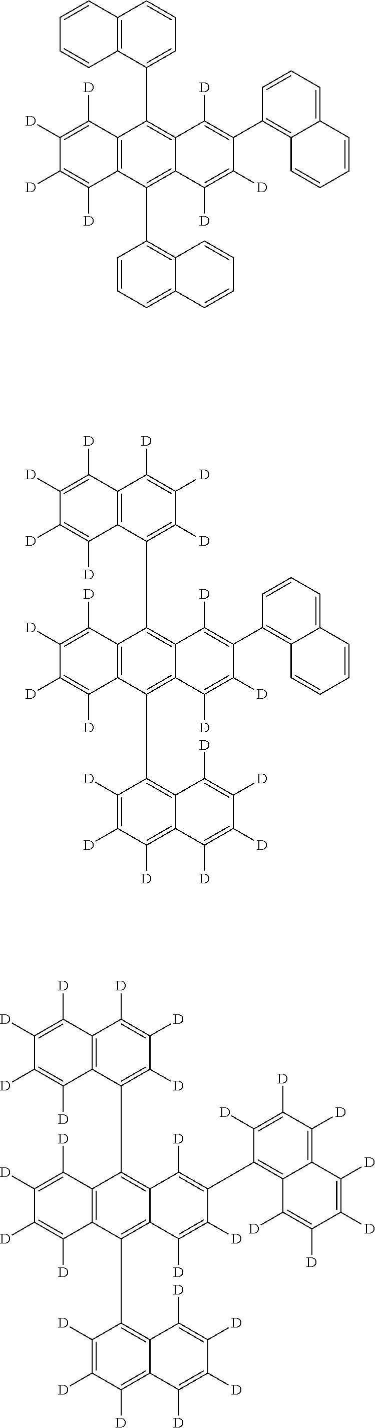

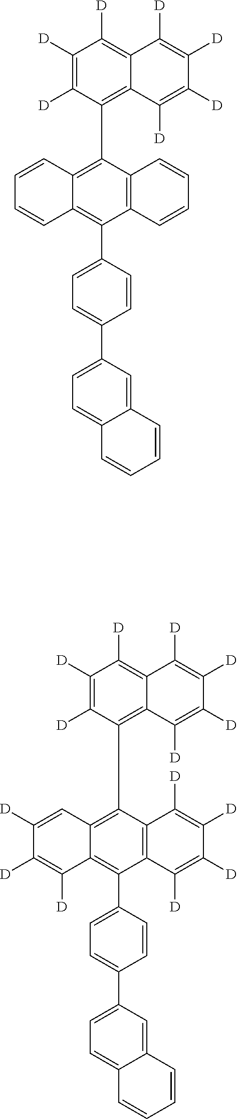 [SMILES: [2H]c1c([2H])c([2H])c2c(-c3c4ccccc4c(-c4ccc(-c5ccc6ccccc6c5)cc4)c4ccccc34)c([2H])c([2H])c([2H])c2c1[2H] ; [2H]c1cc2c(-c3c([2H])c([2H])c([2H])c4c([2H])c([2H])c([2H])c([2H])c34)c3c([2H])c([2H])c([2H])c([2H])c3c(-c3ccc(-c4ccc5ccccc5c4)cc3)c2c([2H])c1[2H]]

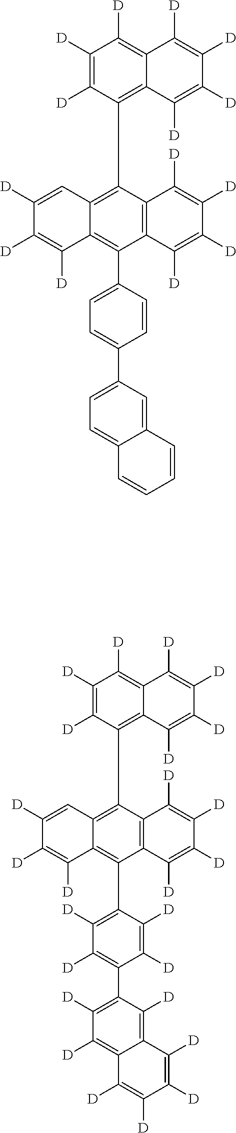 [SMILES: [2H]c1cc2c(-c3c([2H])c([2H])c([2H])c4c([2H])c([2H])c([2H])c([2H])c34)c3c([2H])c([2H])c([2H])c([2H])c3c(-c3ccc(-c4ccc5ccccc5c4)cc3)c2c([2H])c1[2H] ; [2H]c1cc2c(-c3c([2H])c([2H])c([2H])c4c([2H])c([2H])c([2H])c([2H])c34)c3c([2H])c([2H])c([2H])c([2H])c3c(-c3c([2H])c([2H])c(-c4c([2H])c([2H])c5c([2H])c([2H])c([2H])c([2H])c5c4[2H])c([2H])c3[2H])c2c([2H])c1[2H]]

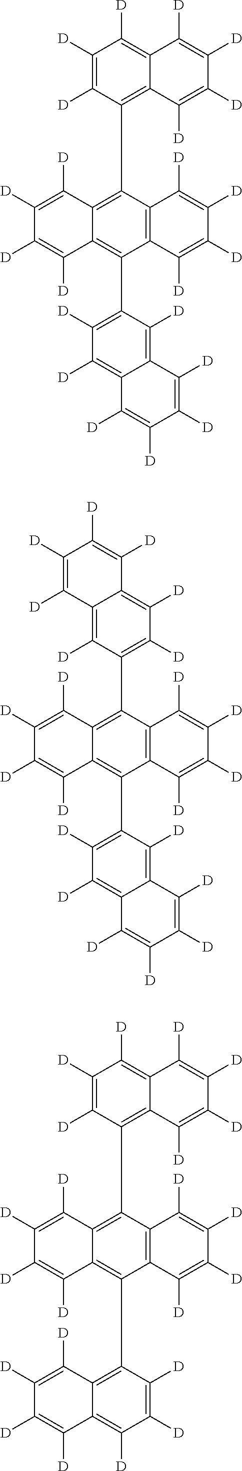

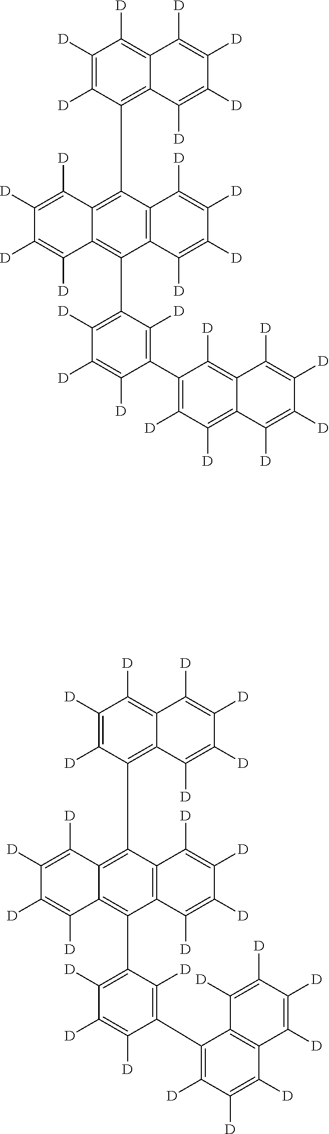 [SMILES: [2H]c1c([2H])c(-c2c([2H])c([2H])c3c([2H])c([2H])c([2H])c([2H])c3c2[2H])c([2H])c(-c2c3c([2H])c([2H])c([2H])c([2H])c3c(-c3c([2H])c([2H])c([2H])c4c([2H])c([2H])c([2H])c([2H])c34)c3c([2H])c([2H])c([2H])c([2H])c23)c1[2H] ; [2H]c1c([2H])c(-c2c([2H])c([2H])c([2H])c3c([2H])c([2H])c([2H])c([2H])c23)c([2H])c(-c2c3c([2H])c([2H])c([2H])c([2H])c3c(-c3c([2H])c([2H])c([2H])c4c([2H])c([2H])c([2H])c([2H])c34)c3c([2H])c([2H])c([2H])c([2H])c23)c1[2H]]

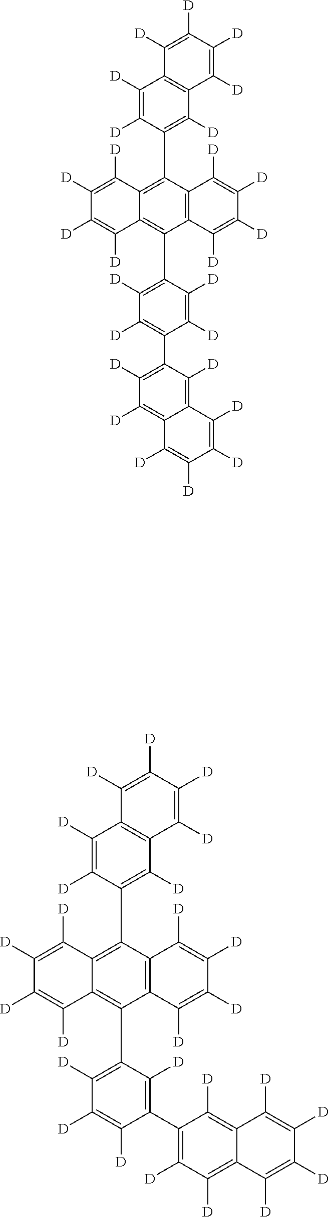 [SMILES: [2H]c1c([2H])c(-c2c3c([2H])c([2H])c([2H])c([2H])c3c(-c3c([2H])c([2H])c4c([2H])c([2H])c([2H])c([2H])c4c3[2H])c3c([2H])c([2H])c([2H])c([2H])c23)c([2H])c([2H])c1-c1c([2H])c([2H])c2c([2H])c([2H])c([2H])c([2H])c2c1[2H] ; [2H]c1c([2H])c(-c2c([2H])c([2H])c3c([2H])c([2H])c([2H])c([2H])c3c2[2H])c([2H])c(-c2c3c([2H])c([2H])c([2H])c([2H])c3c(-c3c([2H])c([2H])c4c([2H])c([2H])c([2H])c([2H])c4c3[2H])c3c([2H])c([2H])c([2H])c([2H])c23)c1[2H]]

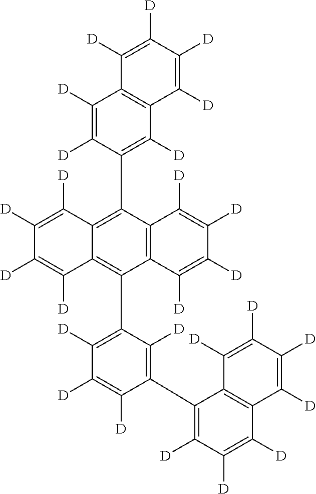 [SMILES: [2H]c1c([2H])c(-c2c([2H])c([2H])c([2H])c3c([2H])c([2H])c([2H])c([2H])c23)c([2H])c(-c2c3c([2H])c([2H])c([2H])c([2H])c3c(-c3c([2H])c([2H])c4c([2H])c([2H])c([2H])c([2H])c4c3[2H])c3c([2H])c([2H])c([2H])c([2H])c23)c1[2H]]

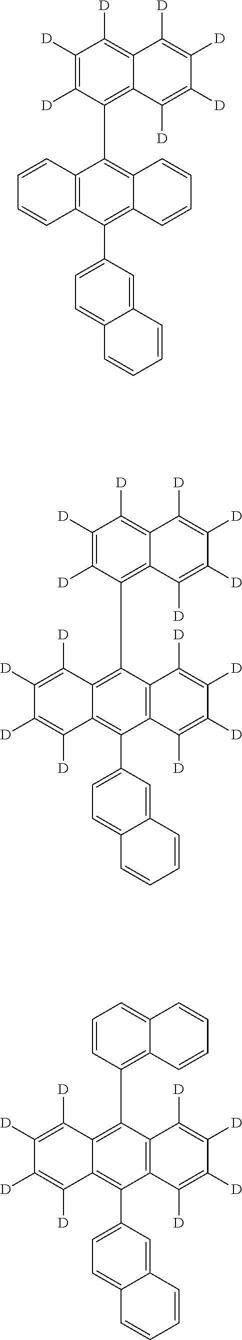 [SMILES: [2H]c1c([2H])c([2H])c2c(-c3c4ccccc4c(-c4ccc5ccccc5c4)c4ccccc34)c([2H])c([2H])c([2H])c2c1[2H] ; [2H]c1c([2H])c([2H])c2c(-c3c4c([2H])c([2H])c([2H])c([2H])c4c(-c4ccc5ccccc5c4)c4c([2H])c([2H])c([2H])c([2H])c34)c([2H])c([2H])c([2H])c2c1[2H] ; [2H]c1c([2H])c([2H])c2c(-c3cccc4ccccc34)c3c([2H])c([2H])c([2H])c([2H])c3c(-c3ccc4ccccc4c3)c2c1[2H]]

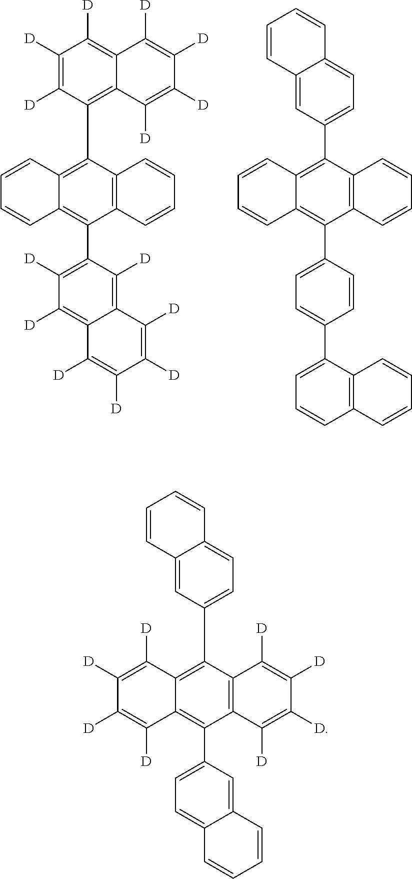

15. The organic light emitting device of claim 11, wherein a weight ratio of the host and the dopant is 99:1 to 90:10.
